(19) United States
(12) Patent Application Publication
(10) Pub. No.: US 2026/0306640 A1
(43) Pub. Date: Oct. 8, 2026

(54) TECHNOLOGIES TARGETING CELL STATES

(71) Applicant: Auron Therapeutics, Inc., Newton, MA (US)

(72) Inventors: James Anthony Neef, Stow, MA (US); David Simon Millan, Stow, MA (US); Andrew John McRiner, Winchester, MA (US); Jake Charles Axford, Waltham, MA (US)

(21) Appl. No.: 19/475,567

(22) PCT Filed: Apr. 19, 2024
(86) PCT No.: PCT/US2024/025468

§ 371 Date: Oct. 15, 2025

Related U.S. Application Data
(60) Provisional application No. 63/460,764, filed on Apr. 20, 2023.
(60) Provisional application No. 63/561,294, filed on Mar. 4, 2024.
(60) Provisional application No. 63/575,594, filed on Apr. 5, 2024.

(30) Foreign Application Priority Data
Sep. 1, 2023 (WO) PCT/US2023/031835

(51) Int. Cl. A61K 47/55; A61K 47/12; A61K 47/40; A61P 35/04; C07D 401/14; C07D 471/10

(52) U.S. Cl. CPC A61K 47/55; A61K 47/12; A61K 47/40; A61P 35/04; C07D 401/14; C07D 471/10

(57) ABSTRACT

Among other things, the present disclosure provides technologies targeting KAT2. In some embodiments, the present disclosure provides technologies for reducing levels of KAT2 polypeptides in various systems. In some embodiments, the present disclosure provides technologies for treating various diseases, disorders, or conditions, including those associated with a KAT2 polypeptide such as various cancers.

### RELATED APPLICATIONS

This application claims priority to U.S. Application No. 63/460,764, filed Apr. 20, 2023, U.S. Application No. 63/561,294, filed Mar. 4, 2024, U.S. Application No. 63/575,594, filed Apr. 5, 2024, and International Application No. PCT/US2023/031835, filed Sep. 1, 2023, the entire contents of each of which are hereby incorporated by reference.

### BACKGROUND

KAT2A (also referred to as general control non-depressible 5 (GCN5)) and KAT2B (also referred to as p300/CBP-associated factor (PCAF)) are both multidomain proteins, which contain an acetyltransferase domain and a bromodomain. KAT2A and KAT2B are reported to function as epigenetic proteins, as they are both reported to be capable of modifying histones and recognizing modified histones. They are reported to be involved in various cellular pathways, including cell proliferation and differentiation, metabolic regulation, and DNA damage repair. See, e.g., Bassi, Z. I., et al. ACS Chem. Biol., 2018, 13, 2862-67. Both KAT2A and KAT2B have been implicated in certain diseases, disorders, and conditions, such as cancers, neurodegenerative diseases, and inflammation. See, e.g., Humphreys, P. G., et al., J. Med. Chem., 2017, 60, 695-709. Conditional knockout of KAT2A in mice bearing an additional mutation that causes acute myeloid leukemia has been reported to delay development of leukemia, deplete leukemia stem cells, and shift leukemia cell fate out of self-renewal into differentiation, causing the disease to become less aggressive. See, e.g., Domingues A. P., et al., eLife 9:e51754, 2020.

### SUMMARY

Among other things, the present disclosure encompasses the recognition that various conditions, disorders or diseases are associated with certain cell states. For example, while many types of normal cells typically go through a proliferative, plastic cell state and then terminally differentiate and are committed to particular cell states and stop proliferating, many cancer cells reside in a plastic and proliferative state (which may be referred to as a plastic state), e.g., through blocking differentiation, de-differentiation, and/or trans-differentiation. In some embodiments, plastic states of cancer cells are associated with aggressive tumors, e.g., aggressive and/or metastatic tumors which can be resistant to reported therapies. In some embodiments, the present disclosure provides technologies for preventing and/or treating conditions, disorders or diseases by targeting their associates cell states. In some embodiments, the present disclosure provides technologies for targeting plastic states of cancer cells. In some embodiments, the present disclosure identifies drivers associated with plastic states of cancer cells. In some embodiments, a driver locks cancer cells in a plastic and proliferative state. In some embodiments, a driver contributes to maintain cancer cells in a plastic and proliferative state. In some embodiments, provided technologies revert cancer cells to normal cell states and/or inhibit their growth and proliferation.

In some embodiments, KAT2A and/or KAT2B are identified as driver(s) for plastic states of various cancer cells, e.g., those of small cell lung cancer, neuroendocrine prostate cancer and acute myeloid leukemia. In some embodiments, the present disclosure provides compounds useful to regulate KAT2 (KAT2A and/or KAT2B), for example to reduce KAT2 activity, for example by degrading (e.g., increasing degradation of) KAT2 (e.g., KAT2A and/or KAT2B). I In some embodiments, provided technologies (e.g., compounds, compositions or methods) can reduce level of a KAT2 protein. In some embodiments, provided technologies can reduce level of KAT2A. In some embodiments, provided technologies can reduce level of KAT2B. In some embodiments, provided technologies can reduce levels of KAT2A and KAT2B. In some embodiments, provided technologies can selectively reduce level of one of KAT2A and KAT2B over the other. In some embodiments, provided technologies can selectively reduce level of KAT2A over KAT2B. In some embodiments, provided compounds are useful for, among other things, treating and/or preventing diseases, disorders, or conditions associated with KAT2 (e.g., KAT2A and/or KAT2B), e.g., with level and/or activity of KAT2 (e.g., KAT2A and/or KAT2B) protein. In some embodiments, the provided technologies provides technologies for preventing or treating conditions, disorders or diseases associated with KAT2A and/or KAT2B. In some embodiments, the provided technologies provides technologies for preventing or treating conditions, disorders or diseases associated with KAT2A. In some embodiments, the provided technologies provides technologies for preventing or treating conditions, disorders or diseases associated with KAT2B.

In some embodiments, provided compounds comprise a protein binding moiety capable of binding a KAT2 protein (e.g., KAT2A and/or KAT2B) and an E3 ligase binding moiety capable of binding an E3 ubiquitin ligase. In some embodiments, a protein binding moiety binds to a bromodomain of KAT2A and/or KAT2B. In some embodiments, provided compounds may recruit a KAT2 protein to an E3 ubiquitin ligase, thereby promoting degradation of (or otherwise inhibiting) the KAT2 protein (e.g., KAT2A and/or KAT2B). In some embodiments, provided compounds promote ubiquitination of KAT2A and/or KAT2B. In some embodiments, provided compounds promote degradation of KAT2A and KAT2B.

In some embodiments, the present disclosure provides a compound of Formula I:

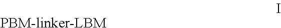

or a pharmaceutically acceptable salt thereof, wherein PBM, linker, and LBM are as defined herein. In some embodiments, a provided compound is a compound of formula II, IIA, IIA-1, IIA-2, IIA-3, IIA-4, IIA-5 or IIA-6, or a salt thereof. In some embodiments, a provided compound is a compound of formula IX, IXA, IXB, IXC or IXD, or a salt thereof.

In some embodiments, a provided compound is a compound of formula III or a salt thereof. In some embodiments, a provided compound is a compound of formula IIIA or a salt thereof. In some embodiments, a provided compound is a compound of formula IIIB, IIIB-1 or IIIB-2, or a salt thereof. In some embodiments, a provided compound is a compound of formula IIIC or a salt thereof. In some embodiments, a provided compound is a compound of formula IIID or a salt thereof. In some embodiments, a provided compound is a compound of formula IV, IVA or IVA-1, or a salt thereof. In some embodiments, a provided compound is a compound of formula V, VA, VA-1, VB or VB-1, or a salt thereof. In some embodiments, a provided compound is a compound of formula VI, VIA or VIA-1, or a salt thereof. In some embodiments, a provided compound is a compound of formula VII or VIIA, or a salt thereof. In some embodiments, a provided compound is a compound of formula VIII or VIIIA, or a salt thereof. In some embodiments, a provided compound is a compound of formula X, XA, XA-1, XA-2 or XB, or a salt thereof. In some embodiments, a provided compound is a compound of formula XI or XIA, or a salt thereof. In some embodiments, a provided compound is a compound of formula XII, XIIA, XIIB or XIIB-1, or a salt thereof. In some embodiments, a provided compound is a compound of formula XIII or XIIIA, or a salt thereof. In some embodiments, a provided compound is a compound of formula XIV or a salt thereof. In some embodiments, a provided compound is a compound of formula XV or a salt thereof. In some embodiments, a salt is a pharmaceutically acceptable salt. In some embodiments, such a compound can bind KAT2A and/or KAT2B, and an E3 ligase. In some embodiments, such a compound promotes ubiquitination of KAT2A and/or KAT2B. In some embodiments, such a compound promotes degradation of KAT2A and/or KAT2B. In some embodiments, such a compound reduces level of KAT2A and/or level of KAT2B. In some embodiments, such a compound is useful for preventing and/or treating a condition, disorder or disease associated with KAT2A and/or KAT2B.

In some embodiments, the present disclosure provides a pharmaceutical composition comprising a provided compound or a pharmaceutically acceptable salt thereof and a pharmaceutically acceptable carrier.

In some embodiments, the present disclosure provides technologies (e.g., compounds or methods) for manufacturing provided compounds.

In some embodiments, the present disclosure provides methods for preventing a condition, disorder or disease comprising administering or delivering to a subject susceptible thereto an effective amount of a provided compound or a pharmaceutically acceptable salt thereof. In some embodiments, the present disclosure provides methods for treating a condition, disorder or disease comprising administering or delivering to a subject suffering therefrom an effective amount of a provided compound or a pharmaceutically acceptable salt thereof. In some embodiments, a pharmaceutical composition is administered to administer or deliver a provided compound or a salt thereof. In some embodiments, a condition, disorder or disease is associated with KAT2A and/or KAT2B. In some embodiments, a condition, disorder or disease is cancer. In some embodiments, a condition, disorder or disease is small-cell lung cancer. In some embodiments, a condition, disorder or disease is neuroendocrine prostate cancer. In some embodiments, a condition, disorder or disease is acute myeloid leukemia.

### BRIEF DESCRIPTION OF DRAWING

FIG. 1. Provided compounds can provide various activities including inhibiting tumor growth in vivo. (A): Provided compounds can inhibit tumor growth in vivo. Nude Balb/c mice were inoculated with NCI-H1048 cells in Matrigel. Tumors were allowed to grow up to about 200 mm³ and mice were then randomized into two groups. Mice were dosed intraperitoneally (i.p.) once per week with vehicle or 30 mg/kg compound 22. Tumor size was measured every three to four days and mice were sacrificed once vehicle tumors reached 2000 mm³. Error bar is SEM. Dosing started at day 17 post tumor inoculation. (B): Provided technologies can reduce KAT2A and KAT2B levels and histone acetylation levels. As shown, western blot data of small cell lung cancer NCI-H1048 cells treated 24 h with compound 22 at various concentrations confirm such reduction. Vehicle is DMSO. (C): Provided technologies can inhibit cell growth. Cell count data relative to DMSO control after 8 days of treatment with compound 22 are presented as examples to confirm inhibition. GI50=6 nM. (D): Provided technologies can inhibit tumor growth in vivo. Nude balb/c mice inoculated with H1048 tumors were dosed intraperitoneally once per week (qw i.p.) with vehicle (HbCD) or compound 22. Average tumor volumes+/−SEM from 6 mice are represented. Significant tumor growth inhibition of 70% at 30 mg/kg was observed and the treatment was well tolerated. See also (A). (E): Provided technologies are well tolerated. Certain body weights relative to starting weight are presented as examples to confirm tolerability and safety. (F): Provided technologies can reduce KAT2A and KAT2B levels and histone acetylation levels in tumor in vivo. Western blots from tumor 168 hours post last dose showed significant reduction of KAT2A and KAT2B and histone acetylation. (G): Provided technologies can modulate cell states and induce differentiation. Box plot of epithelial GSEA demonstrated significant upregulation in tumors treated with compound 22 as compared to vehicle.

FIG. 2. Provided compounds can provide various activities. (A): Provided technologies can reduce KAT2A and KAT2B levels and histone acetylation levels. As shown, western blot data of acute myeloid leukemia MOLM-13 cells treated 48 hour with compound 22 confirm such reduction. (B): Provided technologies can inhibit cell growth. Cell count data after 8 days of treatment with compound 22 relative to DMSO control are presented as examples to confirm inhibition. GI₅₀=1.2 nM. (C): Provided technologies can modulate cell states and induce differentiation. CD86 was analyzed by flow cytometry at 48 hours and monocytic differentiation was observed. EC50=0.3 nM.

FIG. 3. Provided compounds can provide various activities. (A): Provided technologies can reduce KAT2A and KAT2B levels and histone acetylation levels. As shown, western blot of neuroendocrine prostate cancer LASCPC-01 cells treated 24 hours with compound 22 confirm such reduction. (B): Provided technologies can inhibit cell growth. Cell viability (Cell Titer Glo) data relative to DMSO control after 7 days of treatment with compound 22 are presented as examples to confirm inhibition. GI₅₀=5 nM. (C): Provided technologies can modulate cell states and induce differentiation. Box plot of epithelial GSEA demonstrated significant upregulation in cells treated with compound 22 as compared to DMSO. (D): Provided technologies can reduce KAT2A and KAT2B levels and histone acetylation levels including in neuroendocrine prostate cancer primary patient organoid. As shown, western blot of NEPC primary patient organoid treated with compound 22 for 72 hours confirmed such reductions. Concentrations are nM. (E): Provided technologies can inhibit cell growth. Cell viability data relative to DMSO control after 21 days of treatment with compound 22 are presented as examples to confirm inhibition. GI₅₀=3 nM. (F): Provided technologies can modulate cell states and induce differentiation. Box plot of microglial GSEA demonstrated significant upregulation (both epithelial and neural components).

### DETAILED DESCRIPTION OF CERTAIN EMBODIMENTS

Certain provided technologies of the disclosure, e.g., compounds, compositions, methods, include those described generally above, and are further illustrated by the classes, subclasses, and species disclosed herein.

### Definitions

As used herein, the following definitions shall apply unless otherwise indicated. For purposes of this disclosure, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

Unless otherwise indicated, structures depicted herein are meant to represent all stereoisomeric (e.g., enantiomeric or diastereomeric) forms of the structure, as well as all geometric or conformational isomeric forms of the structure. For example, where a stereocenter is present, in some embodiments, the R and/or S configurations of such stereocenter are contemplated as part of the disclosure. Therefore, in some embodiments, single stereochemical isomers, and/or enantiomeric, diastereomic, and/or geometric (or conformational) mixtures of provided compounds are within the scope of the disclosure. For example, in some case, Table 1 and Table 2 show one or more stereoisomers of a compound, and unless otherwise indicated, represents each stereoisomer alone and/or as a mixture. Unless otherwise indicated, tautomeric forms of provided compounds are within the scope of the disclosure.

Unless otherwise indicated, structures depicted herein are meant to include compounds that differ only in the presence of one or more isotopically enriched atoms. For example, compounds having the present structures including replacement of hydrogen by deuterium or tritium, or replacement of a carbon by ¹³C- or ¹⁴C-enriched carbon are within the scope of this disclosure.

Aliphatic: The term “aliphatic” refers to a straight-chain (i.e., unbranched) or branched, optionally substituted hydrocarbon chain that is completely saturated or that contains one or more units of unsaturation, or a monocyclic or bicyclic hydrocarbon that is completely saturated or that contains one or more units of unsaturation but which is not aromatic (also referred to herein as “carbocyclic” or “cycloaliphatic”). Unless otherwise specified, aliphatic groups contain 1-12 aliphatic carbon atoms. In some embodiments, aliphatic groups contain 1-6 aliphatic carbon atoms (e.g., C₁₋₆). In some embodiments, aliphatic groups contain 1-5 aliphatic carbon atoms (e.g., C₁₋₅). In other embodiments, aliphatic groups contain 1-4 aliphatic carbon atoms (e.g., C₁₋₄). In still other embodiments, aliphatic groups contain 1-3 aliphatic carbon atoms (e.g., C₁₋₃), and in yet other embodiments, aliphatic groups contain 1-2 aliphatic carbon atoms (e.g., C₁₋₂). Suitable aliphatic groups include, but are not limited to, linear or branched, substituted or unsubstituted alkyl, alkenyl, alkynyl groups and hybrids thereof. In some embodiments, “aliphatic” refers to a straight-chain (i.e., unbranched) or branched, optionally substituted hydrocarbon chain that is completely saturated or that contains one or more units of unsaturation.

Alkyl: The term “alkyl”, used alone or as part of a larger moiety, refers to a saturated, optionally substituted straight or branched hydrocarbon group having (unless otherwise specified) 1-12, 1-10, 1-8, 1-6, 1-4, 1-3, or 1-2 carbon atoms (e.g., C₁₋₁₂, C₁₋₁₀, C₁₋₈, C₁₋₆, C₁₋₄, C₁₋₃, or C₁₋₂). Exemplary alkyl groups include methyl, ethyl, propyl, butyl, pentyl, hexyl, and heptyl.

Carbocyclyl: The terms “carbocyclyl.” “carbocycle,” and “carbocyclic ring” as used herein, refer to saturated or partially unsaturated cyclic aliphatic monocyclic, bicyclic, or polycyclic ring systems, as described herein, having from 3 to 14 members, wherein the aliphatic ring system is optionally substituted as described herein. Carbocyclic groups include, without limitation, cyclopropyl, cyclobutyl, cyclopentyl, cyclopentenyl, cyclohexyl, cyclohexenyl, cycloheptyl, cycloheptenyl, cyclooctyl, cyclooctenyl, norbornyl, adamantyl, and cyclooctadienyl. In some embodiments, “carbocyclyl” (or “cycloaliphatic”) refers to an optionally substituted monocyclic C₃-C₈ hydrocarbon, or an optionally substituted C₅-C₁₀ (e.g., C₇-C₁₀ or C₈-C₁₀) bicyclic hydrocarbon that is completely saturated or that contains one or more units of unsaturation, but which is not aromatic. The term “cycloalkyl” refers to an optionally substituted saturated ring system of about 3 to about 10 ring carbon atoms. In some embodiments, cycloalkyl groups have 3-6 carbons. Exemplary monocyclic cycloalkyl rings include cyclopropyl, cyclobutyl, cyclopentyl, cyclohexyl, and cycloheptyl. The term “cycloalkenyl” refers to an optionally substituted non-aromatic monocyclic or multicyclic ring system containing at least one carbon-carbon double bond and having about 3 to about 10 carbon atoms. Exemplary monocyclic cycloalkenyl rings include cyclopentenyl, cyclohexenyl, and cycloheptenyl.

Alkenyl: The term “alkenyl!”, used alone or as part of a larger moiety, refers to an optionally substituted straight or branched hydrocarbon chain having at least one double bond and having (unless otherwise specified) 2-12, 2-10, 2-8, 2-6, 2-4, or 2-3 carbon atoms (e.g., C₂₋₁₂, C₂₋₁₀, C₂₋₈, C₂₋₆, C₂₋₄, or C₂₋₃). Exemplary alkenyl groups include ethenyl, propenyl, butenyl, pentenyl, hexenyl, and heptenyl.

Alkynyl: The term “alkynyl”, used alone or as part of a larger moiety, refers to an optionally substituted straight or branched chain hydrocarbon group having at least one triple bond and having (unless otherwise specified) 2-12, 2-10, 2-8, 2-6, 2-4, or 2-3 carbon atoms (e.g., C₂₋₁₂, C₂₋₁₀, C₂₋₈, C₂₋₆, C₂₋₄, or C₂₋₃). Exemplary alkynyl groups include ethynyl, propynyl, butynyl, pentynyl, hexynyl, and heptynyl.

Aryl: The term “aryl” refers to monocyclic and bicyclic ring systems having a total of six to fourteen ring members (e.g., C₆₋₁₄), wherein at least one ring in the system is aromatic and wherein each ring in the system contains three to seven ring members. The term “aryl” may be used interchangeably with the term “aryl ring”. In some embodiments, “aryl” refers to an aromatic ring system which includes, but is not limited to, phenyl, naphthyl, and the like, which may bear one or more substituents. Unless otherwise specified, “aryl” groups are hydrocarbons.

Heteroaliphatic: The term “heteroaliphatic”, as used herein, is given its ordinary meaning in the art and refers to aliphatic groups as described herein in which one or more carbon atoms are independently replaced with one or more heteroatoms (e.g., oxygen, nitrogen, sulfur, silicon, phosphorus, and the like). In some embodiments, one or more units selected from C, CH, CH₂, and CH₃ are independently replaced by one or more heteroatoms (including oxidized and/or substituted forms thereof). In some embodiments, a heteroaliphatic group is heteroalkyl. In some embodiments, a heteroaliphatic group is heteroalkenyl.

Heteroaryl: The terms “heteroaryl” and “heteroar-”, used alone or as part of a larger moiety, e.g., “heteroaralkyl”, or “heteroaralkoxy”, refer to monocyclic or bicyclic ring groups having 5 to 10 ring atoms (e.g., 5- to 6-membered monocyclic heteroaryl or 9- to 10-membered bicyclic heteroaryl); having 6, 10, or 14 π electrons shared in a cyclic array; and having, in addition to carbon atoms, from one to five heteroatoms. Exemplary heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridonyl, pyridazinyl, pyrimidinyl, pyrazinyl, indolizinyl, purinyl, naphthyridinyl, pteridinyl, imidazo[1,2-a]pyrimidinyl, imidazo[1,2-a]pyridinyl, thienopyrimidinyl, triazolopyridinyl, and benzoisoxazolyl. The terms “heteroaryl” and “heteroar-”, as used herein, also include groups in which a heteroaromatic ring is fused to one or more aryl, cycloaliphatic, or heterocyclyl rings. Nonlimiting examples include indolyl, isoindolyl, benzothienyl, benzofuranyl, dibenzofuranyl, indazolyl, benzimidazolyl, benzothiazolyl, benzothiadiazolyl, benzoxazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, pyrido[2,3-b]-1,4-oxazin-3 (4H)-one, and bonzoisoxazolyl. The term “heteroaryl” may be used interchangeably with the terms “heteroaryl ring”, “heteroaryl group”, or “heteroaromatic”, any of which terms include rings that are optionally substituted.

Heteroatom: The term “heteroatom” as used herein refers to nitrogen, oxygen, or sulfur, and includes any oxidized form of nitrogen or sulfur, and any quaternized form of a basic nitrogen.

Heterocycle: As used herein, the terms “heterocycle”, “heterocyclyl”, and “heterocyclic ring” are used interchangeably and refer to a stable 3- to 8-membered monocyclic or 6- to 10-membered bicyclic heterocyclic moiety that is either saturated or partially unsaturated, and having, in addition to carbon atoms, one or more, such as one to four, heteroatoms, as defined above. In some embodiments, they refer to a 3- to 8-membered monocyclic or 7- to 10-membered bicyclic heterocyclic moiety that is either saturated or partially unsaturated, and having, in addition to carbon atoms, one or more, such as one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or NR⁺ (as in N-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothienyl, piperidinyl, decahydroquinolinyl, oxazolidinyl, piperazinyl, dioxanyl, dioxolanyl, diazepinyl, oxazepinyl, thiazepinyl, morpholinyl, and thiamorpholinyl. A heterocyclyl group may be mono-, bi-, tri-, or polycyclic, preferably mono-, bi-, or tricyclic, more preferably mono- or bicyclic. A bicyclic heterocyclic ring also includes groups in which the heterocyclic ring is fused to one or more aryl, heteroaryl, or cycloaliphatic rings. Exemplary bicyclic heterocyclic groups include indolinyl, isoindolinyl, benzodioxolyl, 1,3-dihydroisobenzofuranyl, 2,3-dihydrobenzofuranyl, and tetrahydroquinolinyl. A bicyclic heterocyclic ring can also be a spirocyclic ring system (e.g., 7- to 11-membered spirocyclic fused heterocyclic ring having, in addition to carbon atoms, one or more heteroatoms as defined above (e.g., one, two, three or four heteroatoms)).

Partially Unsaturated: As used herein, the term “partially unsaturated”, when referring to a ring moiety, means a ring moiety that includes at least one double or triple bond between ring atoms. The term “partially unsaturated” is intended to encompass rings having multiple sites of unsaturation, but is not intended to include aromatic (e.g., aryl or heteroaryl) moieties, as herein defined.

Patient or subject: As used herein, the term “patient” or “subject” refers to any organism to which a provided composition is or may be administered, e.g., for experimental, diagnostic, prophylactic, cosmetic, and/or therapeutic purposes. Typical patients or subjects include animals (e.g., mammals such as mice, rats, rabbits, non-human primates, and/or humans). In some embodiments, a patient is a human. In some embodiments, a patient or a subject is suffering from or susceptible to one or more disorders or conditions. In some embodiments, a patient or subject displays one or more symptoms of a disorder or condition. In some embodiments, a patient or subject has been diagnosed with one or more disorders or conditions. In some embodiments, a patient or a subject is receiving or has received certain therapy to diagnose and/or to treat a disease, disorder, or condition.

Specific binding: As used herein, the term “specific binding” refers to an ability to discriminate between possible binding partners in the environment in which binding is to occur. A binding agent that interacts with one particular target when other potential targets are present is said to “bind specifically” to the target with which it interacts. In some embodiments, specific binding is assessed by detecting or determining degree and/or rate of association between the binding agent and its partner; in some embodiments, specific binding is assessed by detecting or determining degree and/or rate of dissociation of a binding agent-partner complex; in some embodiments, specific binding is assessed by detecting or determining ability of the binding agent to compete an alternative interaction between its partner and another entity. In some embodiments, specific binding is assessed by performing such detections or determinations across a range of concentrations.

Substituted or optionally substituted: As described herein, compounds of this disclosure may contain “optionally substituted” moieties. In general, the term “substituted,” whether preceded by the term “optionally” or not, means that one or more hydrogens of the designated moiety are replaced with a suitable substituent (i.e., as described below for optionally substituted groups). “Substituted” applies to one or more hydrogens that are either explicit or implicit from the structure (e.g.,

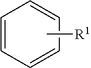

refers to at least

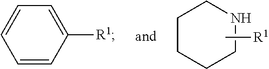

refers to at least

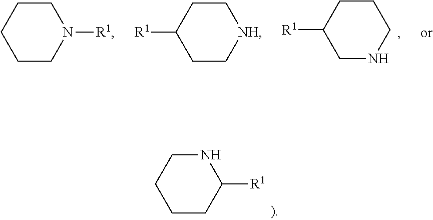

Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes provided herein. Groups described as being “substituted” preferably have between 1 and 4 substituents, more preferably 1 or 2 substituents. Groups described as being “optionally substituted” may be unsubstituted or be “substituted” as described above.

Suitable monovalent substituents on a substitutable carbon atom of an “optionally substituted” group are independently halogen: —(CH₂)₀₋₄R^{∘}; —(CH₂)₀₋₄OR^{∘}; —O(CH₂)₀₋₄R^{∘}; —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR∘₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄SC(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}; —(CH₂)₀₋₄SC(O)R^{∘}; —(CH₂)₀₋₄C(O)NR∘₂; —C(S)NR∘₂; —C(S)SR^{∘}; —SC(S)SR^{∘}, —(CH₂)₀₋₄OC(O)NR∘₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)₂(NH)R^{∘}; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}; —OP(O)R^{∘}₂; —OP(O)(OR^{∘})₂; —SiR^{∘}₃; —(C₁₋₄ straight or branched alkylene) O—N(R^{∘)}₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5- to 6-membered heteroaryl ring), or a 3- to 6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3- to 12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH—)₀₋₂R^{•}, -(haloR^{•}), —(CH₂)₀₋₂OH, (CH₂)₀₋₂OR^{•}, —(CH₂)₀₋₂CH(OR^{•})₂, —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 3- to 6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O (“oxo”), ═S, ═NNR*₂, —NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 3- to 6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 3- to 6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₃, —C(NH)NR^{†}₂, or —N(R^{†})S(O)₂R^{†}; wherein each R^{†} is independently hydrogen, C₁₋₆ aliphatic which may be substituted as defined below, or an unsubstituted 3- to 6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3- to 12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{•}, (haloR®), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 3- to 6-membered saturated, partially unsaturated, or aryl ring having 0-4 heteroatoms independently selected from nitrogen, oxygen, or sulfur.

Treat: As used herein, the term “treat” (also “treatment” or “treating”) refers to any administration of a therapy that partially or completely alleviates, ameliorates, relives, inhibits, delays onset of, reduces severity of, and/or reduces incidence of one or more symptoms, features, and/or causes of a particular disease, disorder, and/or condition. In some embodiments, such treatment may be of a subject who does not exhibit signs of the relevant disease, disorder, and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition.

As used in the present disclosure, unless otherwise clear from context, (i) the term “a” or “an” may be understood to mean “at least one”; (ii) the term “or” may be understood to mean “and/or”; (iii) the terms “comprise”, “comprising”, “include” and “including” (whether used with “not limited to” or not (whether used with “not limited to” or not) may be understood to encompass itemized component(s) or step(s) whether presented by themselves or together with one or more additional components or steps; (iv) the term “another” may be understood to mean at least an additional/second one or more; (v) the terms “about” and “approximately” may be understood to permit standard variation as would be understood by those of ordinary skill in the art; and (vi) where ranges are provided, endpoints are included. Unless otherwise clear from context, isomers of compounds are included. As those skilled in the art appreciate, compounds may be provided, administered, or delivered in various forms, e.g., salts (e.g., pharmaceutically acceptable salts), solvates, hydrates, esters, prodrugs, tautomers, etc.

### Certain Compounds

In some embodiments, the present disclosure provides compounds useful for preventing or treating various conditions, disorders or diseases including cancer. In some embodiments, the present disclosure provides compounds useful for inhibiting cell proliferation, e.g., cancer cell proliferation. In some embodiments, the present disclosure provides compounds useful for reducing tumor growth. In some embodiments, the present disclosure provides compounds useful for reducing level of KAT2A and/or level of KAT2B. In some embodiments, the present disclosure provides compounds useful for inhibiting one or more functions of KAT2A and/or KAT2B. In some embodiments, the present disclosure provides compounds that can bind to KAT2A and/or KAT2B, and can bind to an E3 ligase. Certain compounds are described below as examples.

In some embodiments, the present disclosure provides KAT2 degrader compounds, comprising a KAT2 protein binding moiety, a linker, and an E3 ubiquitin ligase binding moiety.

In some embodiments, the present disclosure provides a compound of Formula I:

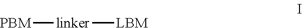

or a pharmaceutically acceptable salt thereof, wherein:
PBM is a KAT2 protein binding moiety, or is

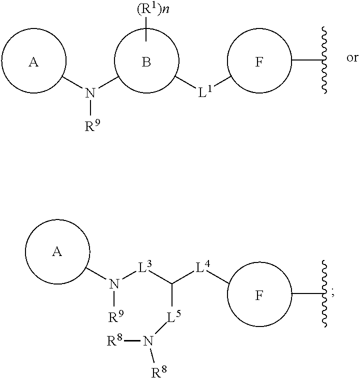

linker is an optional linking moiety, or is a covalent bond or an optionally substituted bivalent C₁-C₂₀ hydrocarbon chain, wherein one or more methylene units are optionally and independently replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —S(O)—, —SO₂—, —SO₂N(R)—, —N(R)SO₂—, or -Cy-; and
LBM is an E3 ubiquitin ligase binding moiety, or is

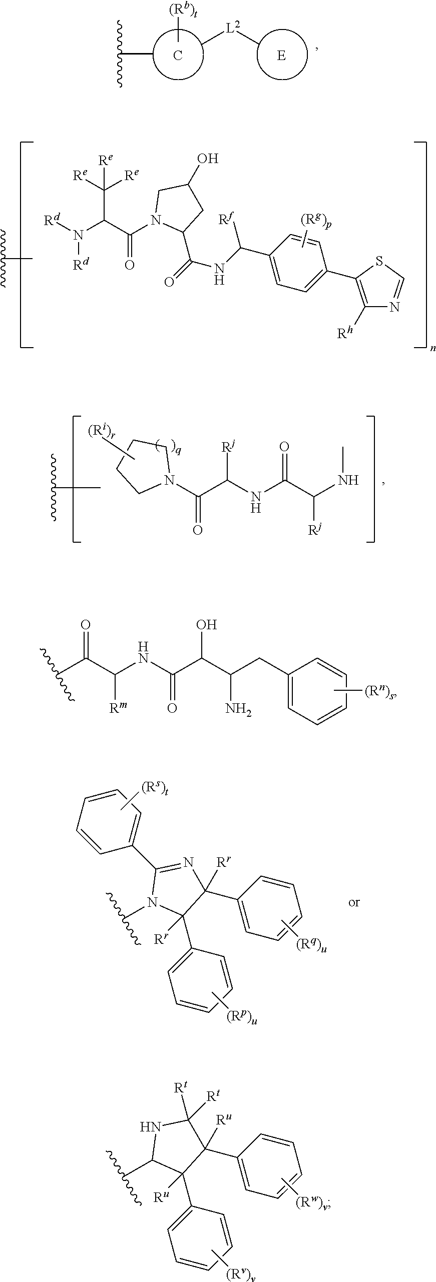

wherein:
Ring A is selected from:

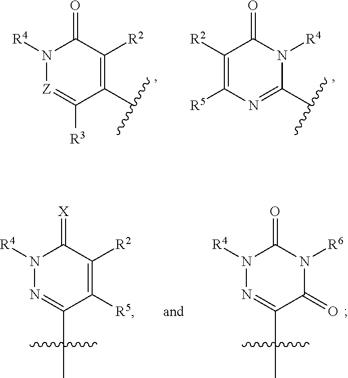

Ring B is an optionally substituted 5- to 6-membered heterocyclyl having 1-2 heteroatoms independently selected from N, O, and S;
L¹ is a covalent bond or an optionally substituted bivalent C₁₋₃ hydrocarbon chain;
each R¹ is independently —R;
n is 0, 1, 2, 3, or 4;
Z is N or CR³;
R² is halogen, —CN, or —R;
each R³ is independently halogen, —CN, or —R;
R⁴ is —R′;
R⁵ is halogen, —CN, or —R, or
   R² and R⁵, together with the atoms to which they are attached, combine to form an optionally substituted 5- to 6-membered ring having 0-2 heteroatoms independently selected from N, O, and S;
R⁶ is —R′;
X is O or NR⁷;
R⁷ is —R′, or
   R⁴ and R⁷, together with the atoms to which they are attached, combine to form an optionally substituted 5- to 6-membered ring having 2-3 heteroatoms independently selected from N, O, and S;
L³, L⁴, and L⁵ are each independently a covalent bond or an optionally substituted bivalent C₁₋₆ hydrocarbon chain;
each R⁸ is independently —R′;
R⁹ is —R′;
each Cy is independently an optionally substituted, 3- to 16-membered bivalent ring having 0-6 heteroatoms independently selected from N, O, and S;
each of Ring C and Ring F is independently an optionally substituted 3- to 16-membered ring having 0-6 heteroatoms independently selected from N, O, and S;
each R^{b} is independently halogen, —CN, —R′ or —OR;
t is 0, 1, 2, 3, or 4;
L² is a covalent bond or an optionally substituted C₁₋₃ hydrocarbon chain, wherein one or more methylene units are optionally and independently replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —S(O)—, —SO₂—, —SO₂N(R)—, —N(R)SO₂—, or -Cy-;
Ring E is

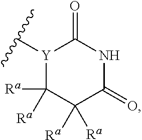

or an optionally substituted group selected from

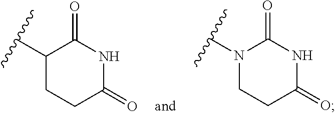 [SMILES: *C1CCC(=O)NC1=O ; "and" ; *N1CCC(=O)NC1=O]

Y is N or CH;
each R^{a} is independently halogen, —CN or —R′;
linker is attached to the bracketed moiety at one of R^{d}, R^{e}, R^{f}, or R^{g}, or linker is attached to the bracketed moiety at one Rⁱ or the ring formed when two Rⁱ groups are taken together;
each R^{d} that is not the point of attachment for the linker is independently —R′, or
   two R^{d}, together with the atom to which they are attached, combine to form an optionally substituted 5- to 6-membered ring having 1-3 heteroatoms independently selected from N, O, and S and optionally fused to a phenyl or 5- to 6-membered heteroaryl ring having 1-3 heteroatoms independently selected from N, O, and S;
each R^{e} that is not the point of attachment for the linker is independently —R;
R^{f}, when it is not the point of attachment for the linker, is —R;
each R^{g} that is not the point of attachment for the linker is independently halogen, —CN, R or —OR;
p is 0, 1, 2, or 3;
R^{h} is halogen, or —R;
each Rⁱ that is not the point of attachment of the linker is independently halogen, —R, —C(O)N(R′)₂, or —N(R)C(O)R, or
   two Rⁱ groups, together with the atoms to which they are attached, combine to form an optionally substituted phenyl or 5- to 6-membered ring having 1-3 heteroatoms independently selected from N, O, and S;
R^{j} is —R, or
   R^{j} combines with one instance of Rⁱ, together with their intervening atoms, to form an optionally substituted 5- to 7-membered ring having 1-2 heteroatoms independently selected from N, O, and S;
R^{k} is —R;
r is 1, 2, 3, 4, or 5;
q is 1 or 2;
R^{m} is —R;
each of Rⁿ, R^{p}, R^{q}, R^{r}, R^{s}, and R^{u} is independently halogen, —CN, —R or —OR;
s is 0, 1, 2, 3, 4, or 5;
t is 0, 1, 2, 3, 4, or 5;
each u is independently 0, 1, 2, 3, 4, or 5;
each R^{t} is independently R, or
   both R^{t} groups, together with the atom to which they are attached, combine to form an optionally substituted 3- to 7-membered ring having 0-2 heteroatoms independently selected from N, O, and S;
each R^{v} is independently halogen, —CN, —R or —OR, or
   one instance of R^{u} and R^{v}, together with their intervening atoms, combine to form an optionally substituted 3- to 7-membered ring having 0-2 heteroatoms independently selected from N, O, and S;
each R^{w} is independently halogen, —CN, —R or —OR, or
   one instance of R^{u} and R^{w}, together with their intervening atoms, combine to form an optionally substituted 3- to 7-membered ring having 0-2 heteroatoms independently selected from N, O, and S;
each v is independently 0, 1, 2, 3, 4, or 5;
each R′ is independently —R, —C(O)R, or —S(O)₂R, or two R′ attached to the same atom, together with the atom to which they are attached, combine to form an optionally substituted 3- to 16-membered ring having 1-5 heteroatoms independently selected from N, O, and S; and
each R is independently hydrogen or an optionally substituted group selected from C₁₋₈ aliphatic, C₃₋₁₀ cycloaliphatic, C₁-C₈ heteroaliphatic having 1-3 heteroatoms independently selected from N, O, and S, C₆₋₁₀ aryl, 5- to 10-membered heteroaryl having 1-5 heteroatoms independently selected from N, O, and S, and 3- to 10-membered monocyclic heterocyclyl having 1-5 heteroatoms independently selected from N, O, and S.

In some embodiments, the present disclosure provides a compound of Formula I:

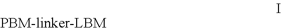

or a pharmaceutically acceptable salt thereof, wherein:
PBM is a KAT2 protein binding moiety;
linker is an optional linking moiety; and
LBM is an E3 ubiquitin ligase binding moiety.

In some embodiments, PBM is

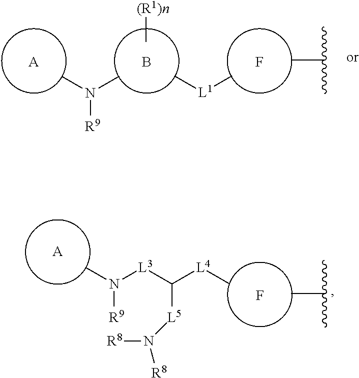

### and LBM is

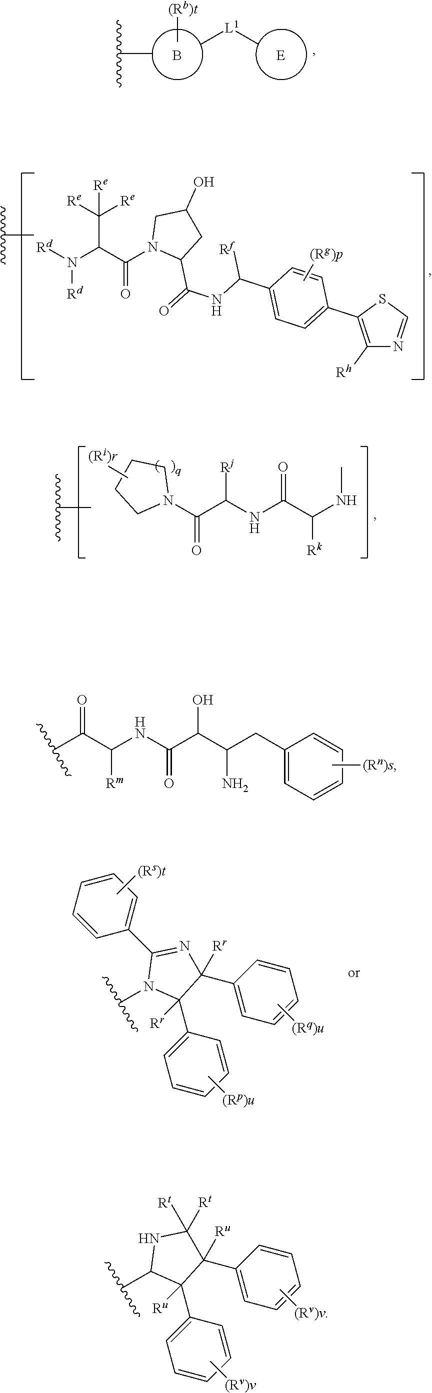

In some embodiments, PBM is

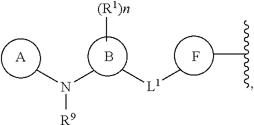

### and LBM is

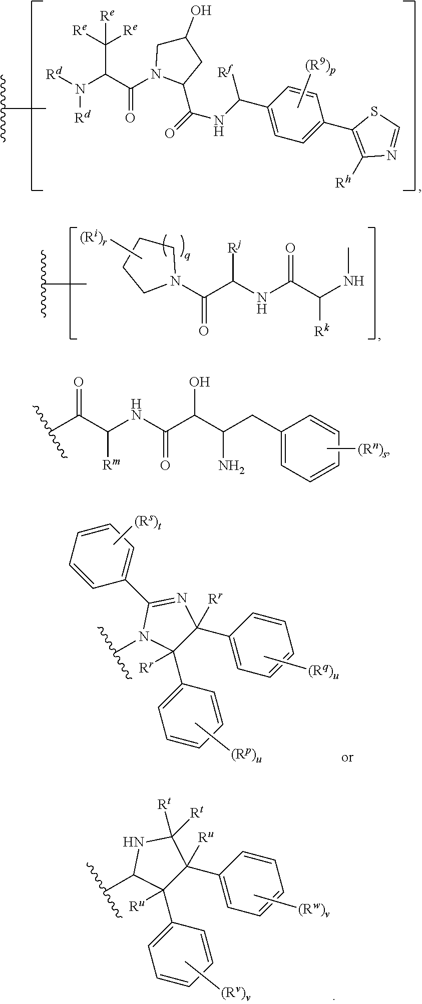

In some embodiments, PBM is

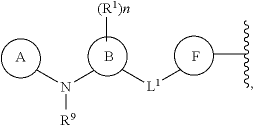

### and LBM is

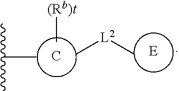

In some embodiments, a compound comprises a moiety, e.g., PBM, that can bind a KAT2 protein or a fragment (e.g., bromodomain) thereof. In some embodiments, PBM-H or a salt thereof can bind a KAT2 protein or a fragment (e.g., bromodomain) thereof. In some embodiments. PBM-H or a salt thereof binds KAT2A or a fragment (e.g., bromodomain) thereof. In some embodiments, PBM-H or a salt thereof binds KAT2B or a fragment (e.g., bromodomain) thereof. In some embodiments, PBM-H or a salt thereof binds KAT2A or a fragment (e.g., bromodomain) thereof, and KAT2B or a fragment (e.g., bromodomain) thereof. In some embodiments, PBM-linker-H or a salt thereof can bind a KAT2 protein or a fragment (e.g., bromodomain) thereof. In some embodiments, PBM-linker-H or a salt thereof binds KAT2A or a fragment (e.g., bromodomain) thereof. In some embodiments, PBM-linker-H or a salt thereof binds KAT2B or a fragment (e.g., bromodomain) thereof. In some embodiments, PBM-linker-H or a salt thereof binds KAT2A or a fragment (e.g., bromodomain) thereof, and KAT2B or a fragment (e.g., bromodomain) thereof. In some embodiments, H-LBM or a salt thereof can bind an E3 ligase (e.g., CRBN, VHL, IAP, or MDM2). In some embodiments, H-LBM or a salt thereof bind CRBN. In some embodiments, H-LBM or a salt thereof bind VHL. In some embodiments. H-linker-LBM or a salt thereof can bind an E3 ligase (e.g., CRBN, VHL, IAP, or MDM2). In some embodiments, H-linker-LBM or a salt thereof bind CRBN. In some embodiments. H-linker-LBM or a salt thereof bind VHL.

In some embodiments, the present disclosure provides a compound of Formula II:

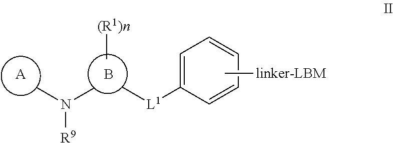

or a pharmaceutically acceptable salt thereof, wherein:
Ring A is selected from:

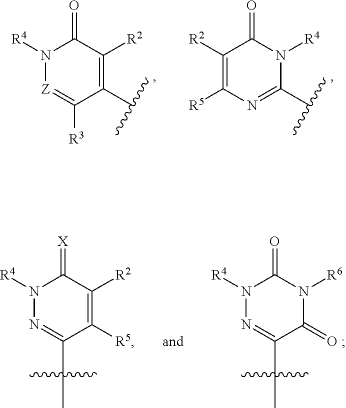

Ring B is a 5- to 6-membered heterocyclyl having 1-2 heteroatoms independently selected from N, O, and S;
L¹ is a covalent bond or a bivalent C₁₋₃ straight or branched hydrocarbon chain;
each R¹ is independently optionally substituted C₁₋₆ aliphatic or optionally substituted C₃₋₆ cycloaliphatic;
n is 0, 1, 2, 3, or 4;
Z is N or CR³;
R² is hydrogen, halogen, —CN, optionally substituted C₁₋₆ aliphatic, or optionally substituted C₃₋₆ cycloaliphatic;
each R³ is independently hydrogen, halogen, optionally substituted C₁₋₆ aliphatic, or optionally substituted C₃₋₆ cycloaliphatic;
R⁴ is hydrogen, optionally substituted C₁₋₆ aliphatic, or optionally substituted C₃₋₆ cycloaliphatic;
R⁵ is hydrogen, halogen, optionally substituted C₁₋₆ aliphatic, or optionally substituted C₃₋₆ cycloaliphatic, or
   R² and R⁵, together with the atoms to which they are attached, combine to form an optionally substituted 5- to 6-membered aromatic ring having 0-2 heteroatoms independently selected from N, O, and S;
R⁶ is hydrogen, optionally substituted C₁₋₆ aliphatic, or optionally substituted C₃₋₆ cycloaliphatic;
X is O or NR⁷;
R⁷ is hydrogen or optionally substituted C₁₋₆ aliphatic, or
   R⁴ and R⁷, together with the atoms to which they are attached, combine to form an optionally substituted 5- to 6-membered aromatic ring having 2-3 heteroatoms independently selected from N, O, and S;
R⁹ is hydrogen or optionally substituted C₁₋₆ aliphatic;
linker is a covalent bond or an optionally substituted, bivalent, straight or branched, saturated or unsaturated C₁-C₂₀ hydrocarbon chain, wherein one or more methylene units are optionally and independently replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —SO₂—, —SO₂N(R)—, —N(R)SO₂—, or -Cy-;
each Cy is independently an optionally substituted, mono- or multicyclic, 3- to 16-membered bivalent ring system, wherein the ring system is fully saturated, partially saturated, or aromatic, and the ring system contains 0-6 heteroatoms independently selected from N, O, and S;
each R is independently hydrogen or an optionally substituted group selected from C₁₋₆ aliphatic, phenyl, C₃₋₇ cycloaliphatic, 5- to 6-membered monocyclic heteroaryl having 1-4 heteroatoms independently selected from N, O, and S, and a 3- to 7-membered monocyclic heterocyclyl having 1-2 heteroatoms independently selected from N, O, and S; and
LBM is an E3 ubiquitin ligase binding moiety.

In some embodiments, the present disclosure provides a compound of Formula II, or a pharmaceutically acceptable salt thereof, wherein:
Ring A is selected from:

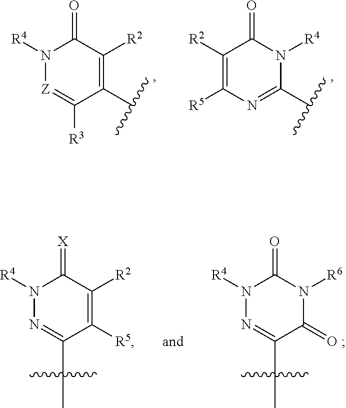

Ring B is a 5- to 6-membered heterocyclyl having 1-2 heteroatoms independently selected from N, O, and S;
L¹ is a covalent bond or a bivalent C₁₋₃ straight or branched hydrocarbon chain;
each R¹ is independently optionally substituted C₁₋₆ aliphatic, or optionally substituted C₃₋₆ cycloaliphatic;
n is 0, 1, 2, 3, or 4;
Z is N or CR³;
R² is hydrogen, halogen, optionally substituted C₁₋₆ aliphatic, or optionally substituted C₃₋₆ cycloaliphatic;
each R³ is independently hydrogen, halogen, optionally substituted C₁₋₆ aliphatic, or optionally substituted C₃₋₆ cycloaliphatic;
R⁴ is hydrogen, optionally substituted C₁₋₆ aliphatic, or optionally substituted C₃₋₆ cycloaliphatic;
R⁵ is hydrogen, halogen, optionally substituted C₁₋₆ aliphatic, or optionally substituted C₃₋₆ cycloaliphatic, or
   R² and R⁵, together with the atoms to which they are attached, combine to form an optionally substituted 5- to 6-membered aromatic ring having 0-2 heteroatoms independently selected from N, O, and S;
R⁶ is hydrogen, optionally substituted C₁₋₆ aliphatic, or optionally substituted C₃₋₆ cycloaliphatic;
X is O or NR⁷;
R⁷ is hydrogen or optionally substituted C₁₋₆ aliphatic, or
R⁴ and R⁷, together with the atoms to which they are attached, combine to form an optionally substituted 5- to 6-membered aromatic ring having 2-3 heteroatoms independently selected from N, O, and S;
R⁹ is hydrogen or optionally substituted C₁₋₆ aliphatic;
linker is a covalent bond or an optionally substituted, bivalent, straight or branched, saturated or unsaturated C₁-C₂₀ hydrocarbon chain, wherein one or more methylene units are optionally and independently replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —SO₂—, —SO₂N(R)—, —N(R)SO₂—, or -Cy-;
each Cy is independently an optionally substituted, mono- or multicyclic, 3- to 16-membered bivalent ring system, wherein the ring system is fully saturated, partially saturated, or aromatic, and the ring system contains 0-6 heteroatoms independently selected from N, O, and S;
each R is independently hydrogen or an optionally substituted group selected from C₁₋₆ aliphatic, phenyl, C₃₋₇ cycloaliphatic, 5- to 6-membered monocyclic heteroaryl having 1-4 heteroatoms independently selected from N, O, and S, and a 3- to 7-membered monocyclic heterocyclyl having 1-2 heteroatoms independently selected from N, O, and S; and
LBM is an E3 ubiquitin ligase binding moiety.

In some embodiments, the present disclosure provides a compound of Formula IIA:

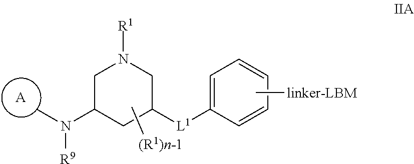

or a pharmaceutically acceptable salt thereof, wherein Ring A, L¹, R¹, R⁹, n, linker, and LBM are as defined above for Formula II and described in classes and subclasses herein, both singly and in combination.

In some embodiments, the present disclosure provides a compound of Formula IIA-1, IIA-2, IIA-3, or IIA-4:

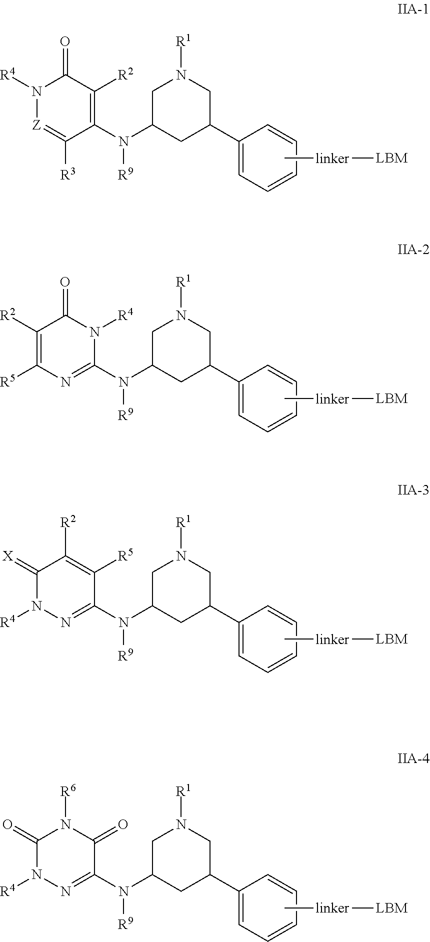

or a pharmaceutically acceptable salt thereof, wherein R¹, R², R³, R⁴, R⁵, R⁶, R⁹, X, Z, linker, and LBM are as defined above for Formula II and described in classes and subclasses herein, both singly and in combination. In some embodiments, the present disclosure provides a compound of Formula IIA-1, or a pharmaceutically acceptable salt thereof. In some embodiments, the present disclosure provides a compound of Formula IIA-2, or a pharmaceutically acceptable salt thereof. In some embodiments, the present disclosure provides a compound of Formula IIA-3, or a pharmaceutically acceptable salt thereof. In some embodiments, the present disclosure provides a compound of Formula IIA-4, or a pharmaceutically acceptable salt thereof.

In some embodiments, the present disclosure provides a compound of Formula IIA-1:

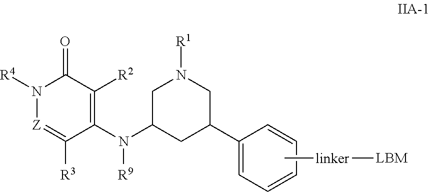

or a pharmaceutically acceptable salt thereof, wherein:
R¹ is optionally substituted C₁₋₆ aliphatic;
Z is N or CR³;
R² is hydrogen, halogen, —CN, optionally substituted C₁₋₆ aliphatic, or optionally substituted C₃₋₆ cycloaliphatic;
each R³ is independently hydrogen or halogen;
R⁴ is hydrogen, optionally substituted C₁₋₆ aliphatic, or optionally substituted C₃₋₆ cycloaliphatic;
R⁹ is hydrogen;
linker is an optionally substituted, bivalent, straight or branched, saturated or unsaturated C₁-C₁₀ hydrocarbon chain, wherein one or more methylene units are replaced by —O—, —N(R)—, —C(O)—, —OC(O)—, —C(O)O—, —C(O)N(R)—, —N(R)C(O)—, or -Cy-;
each Cy is independently an optionally substituted group selected from phenyl, 5- to 6-membered monocyclic heteroaryl having 1-4 heteroatoms independently selected from N, O, and S, monocyclic 4- to 7-membered heterocyclyl having 1-2 heteroatoms independently selected from N, O, and S, and bicyclic 6- to 11-membered heterocyclyl having 1-3 heteroatoms independently selected from N, O, and S;
each R is independently hydrogen or optionally substituted C₁₋₆ aliphatic;
LBM is

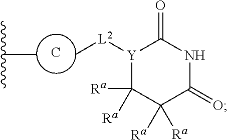

Ring C is an optionally substituted group selected from phenyl, 5- to 6-membered heteroaryl having 1-4 heteroatoms independently selected from N, O, and S, and 9- to 10-membered bicyclic heteroaryl having 1-4 heteroatoms independently selected from N, O, and S;
each R^{a} is hydrogen;
L² is a covalent bond, —CH₂—, —O—, or —N(R)—; and
Y is N or CH.

In some embodiments, the present disclosure provides a compound of Formula IIA-1:

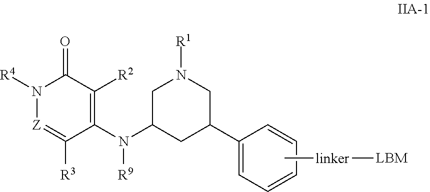

or a pharmaceutically acceptable salt thereof, wherein:
R¹ is C₁₋₆ alkyl;
Z is N or CR³;
R² is hydrogen, halogen, —CN, C₁₋₆ alkyl, or C₃₋₆ cycloalkyl;
each R³ is independently hydrogen or halogen;
R⁴ is C₁₋₆ alkyl;
R⁹ is hydrogen;
linker is an optionally substituted, bivalent, straight or branched, saturated or unsaturated C₁-C₁₀ hydrocarbon chain, wherein one or more methylene units are replaced by —O—, —N(R)—, —C(O)—, —OC(O)—, —C(O)O—, —C(O)N(R)—, —N(R)C(O)—, or -Cy-;
each Cy is independently an optionally substituted group selected from phenyl, 5- to 6-membered monocyclic heteroaryl having 1-4 heteroatoms independently selected from N, O, and S, monocyclic 4- to 7-membered heterocyclyl having 1-2 heteroatoms independently selected from N, O, and S, and bicyclic 6- to 11-membered heterocyclyl having 1-3 heteroatoms independently selected from N, O, and S;
each R is independently hydrogen or C₁₋₆ alkyl;
LBM is

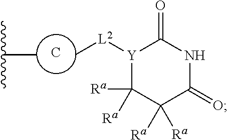

Ring C is an optionally substituted group selected from phenyl, 5- to 6-membered heteroaryl having 1-4 heteroatoms independently selected from N, O, and S, and 9- to 10-membered bicyclic heteroaryl having 1-4 heteroatoms independently selected from N, O, and S;
each R^{a} is hydrogen.
L² is a covalent bond, —CH₂—, —O—, or —N(R)—; and
Y is N or CH.

In some embodiments, the present disclosure provides a compound of Formula IIA-5;

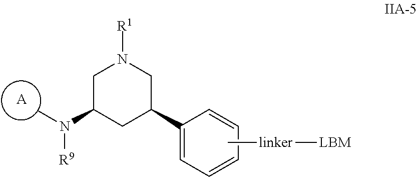

or a pharmaceutically acceptable salt thereof, wherein Ring A, R¹, R⁹, linker, and LBM are as defined above for Formula II and described in classes and subclasses herein, both singly and in combination.

In some embodiments, the present disclosure provides a compound of Formula IIA-6:

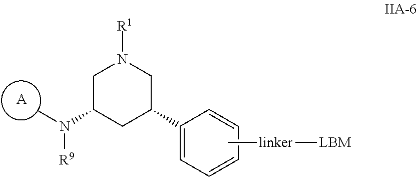

or a pharmaceutically acceptable salt thereof, wherein Ring A, R¹, R⁹, linker, and LBM are as defined above for Formula II and described in classes and subclasses herein, both singly and in combination.

In some embodiments, the present disclosure provides a compound of Formula II-1:

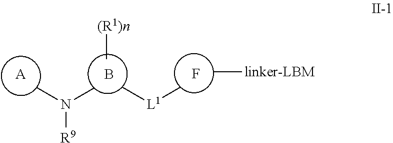

or a pharmaceutically acceptable salt thereof, wherein:
Ring A is selected from:

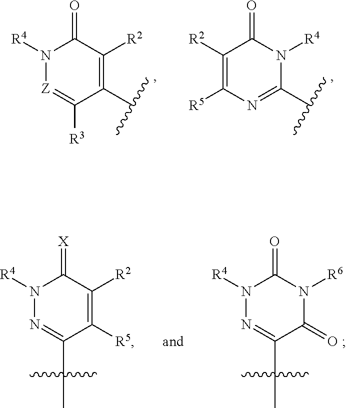

Ring B is an optionally substituted 5- to 6-membered heterocyclyl having 1-2 heteroatoms independently selected from N, O, and S;
L¹ is a covalent bond or an optionally substituted bivalent C₁₋₃ hydrocarbon chain;
each R¹ is independently —R;
n is 0, 1, 2, 3, or 4;
Z is N or CR³;
R² is halogen, —CN, or —R;
each R³ is independently halogen, —CN, or —R;
R⁴ is —R′;
R⁵ is halogen, —CN, or —R, or
   R² and R⁵, together with the atoms to which they are attached, combine to form an optionally substituted 5- to 6-membered ring having 0-2 heteroatoms independently selected from N, O, and S;
R⁶ is —R′;
X is O or NR⁷;
R⁷ is —R′, or
   R⁴ and R⁷, together with the atoms to which they are attached, combine to form an optionally substituted 5- to 6-membered ring having 2-3 heteroatoms independently selected from N, O, and S;
R⁹ is —R′;
linker is a covalent bond or an optionally substituted bivalent C₁-C₂₀ hydrocarbon chain, wherein one or more methylene units are optionally and independently replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —S(O)—, —SO₂—, —SO₂N(R)—, —N(R)SO₂—, or -Cy-;
each Cy is independently an optionally substituted, 3- to 16-membered bivalent ring having 0-6 heteroatoms independently selected from N, O, and S;
Ring F is an optionally substituted 3- to 16-membered ring having 0-6 heteroatoms independently selected from N, O, and S;
LBM is an E3 ubiquitin ligase binding moiety;
each R′ is independently —R, —C(O)R, or —S(O)₂R, or two R′ attached to the same atom, together with the atom to which they are attached, combine to form an optionally substituted 3- to 16-membered ring having 1-5 heteroatoms independently selected from N, O, and S; and
each R is independently hydrogen or an optionally substituted group selected from C₁₋₈ aliphatic, C₃₋₁₀ cycloaliphatic, C₁-C₈ heteroaliphatic having 1-3 heteroatoms independently selected from N, O, and S, C₆₋₁₀ aryl, 5- to 10-membered heteroaryl having 1-5 heteroatoms independently selected from N, O, and S, and 3- to 10-membered monocyclic heterocyclyl having 1-5 heteroatoms independently selected from N, O, and S.

In some embodiments, the present disclosure provides a compound of Formula III:

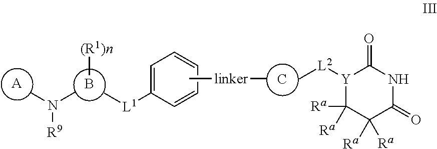

or a pharmaceutically acceptable salt thereof, wherein:
Ring A is selected from:

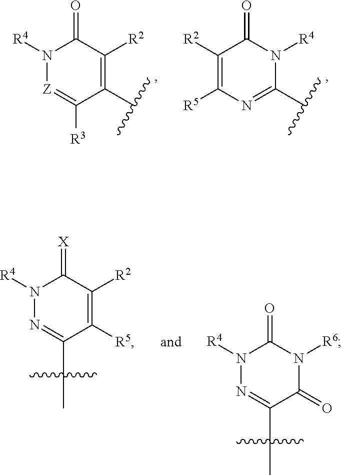

Ring B is a 5- to 6-membered heterocyclyl having 1-2 heteroatoms independently selected from N, O, and S;
L¹ is a covalent bond or a bivalent C₁₋₃ straight or branched hydrocarbon chain;
each R¹ is independently optionally substituted C₁₋₆ aliphatic or optionally substituted C₃₋₆ cycloaliphatic;
n is 0, 1, 2, 3, or 4;
Z is N or CR³;
R² is hydrogen, halogen, —CN, optionally substituted C₁₋₆ aliphatic, or optionally substituted C₃₋₆ cycloaliphatic;
each R³ is independently hydrogen, halogen, optionally substituted Che aliphatic, or optionally substituted C₃₋₆ cycloaliphatic;
R⁴ is hydrogen, optionally substituted C₁₋₆ aliphatic, or optionally substituted C₃₋₆ cycloaliphatic;
R⁵ is hydrogen, halogen, optionally substituted C₁₋₆ aliphatic, or optionally substituted C₃₋₆ cycloaliphatic, or
   R² and R⁵, together with the atoms to which they are attached, combine to form an optionally substituted 5- to 6-membered aromatic ring having 0-2 heteroatoms independently selected from N, O, and S;
R⁶ is hydrogen, optionally substituted C₁₋₆ aliphatic, or optionally substituted C₃₋₆ cycloaliphatic;
X is O or NR⁷;
R⁷ is hydrogen or optionally substituted C₁₋₆ aliphatic, or
   R⁴ and R⁷, together with the atoms to which they are attached, combine to form an optionally substituted 5- to 6-membered aromatic ring having 2-3 heteroatoms independently selected from N, O, and S;

R⁹ is hydrogen or optionally substituted C₁₋₆ aliphatic;

linker is a covalent bond or an optionally substituted, bivalent, straight or branched, saturated or unsaturated C₁-C₂₀ hydrocarbon chain, wherein one or more methylene units are optionally and independently replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —SO₂, —SO₂N(R)—, —N(R)SO₂—, or -Cy-;
each Cy is independently an optionally substituted, mono- or multicyclic, 3- to 16-membered bivalent ring system, wherein the ring system is fully saturated, partially saturated, or aromatic, and the ring system contains 0-6 heteroatoms independently selected from N, O, and S;
Ring C is an optionally substituted, mono- or multicyclic, 3- to 16-membered bivalent ring system, wherein the ring system is fully saturated, partially saturated, or aromatic, and the ring system contains 0-6 heteroatoms independently selected from N, O, and S;
L² is a covalent bond or a straight or branched C₁₋₃ hydrocarbon chain wherein one methylene is optionally replaced with —O—, —S—, —N(R)—, —SO₂—, —C(O)N(R)—, or —N(R)C(O)—;
Y is N or CH;
each R^{a} is independently hydrogen or an optionally substituted C₁₋₆ aliphatic, or two R^{a} groups, together with the atom(s) to which they are attached, combine to form a 3- to 6-membered saturated or partially unsaturated ring;
each R is independently hydrogen or an optionally substituted group selected from C₁₋₆ aliphatic, phenyl, C₃₋₇ cycloaliphatic, 5- to 6-membered monocyclic heteroaryl having 1-4 heteroatoms independently selected from N, O, and S, and a 3- to 7-membered monocyclic heterocyclyl having 1-2 heteroatoms independently selected from N, O, and S; and
Ring A, Ring B, L¹, R¹, R⁹, n, and linker are as defined above for Formula II and described in classes and subclasses herein, both singly and in combination.

In some embodiments, the present disclosure provides a compound of Formula IIIA:

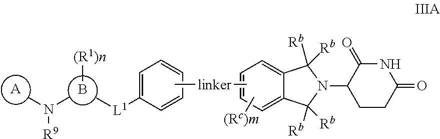

or a pharmaceutically acceptable salt thereof, wherein Ring A. Ring B, L¹, R¹, R⁹, n, and linker are as defined above for Formula II and described in classes and subclasses herein, both singly and in combination; and
each R^{b} is hydrogen, or two R^{b} groups, on the same carbon, are taken together to form an oxo or combine to form a 3- to 6-membered saturated or partially unsaturated ring;
each R^{e} is independently selected from halogen, —OR, —N(R)₂, —CN, and optionally substituted C₁₋₆ aliphatic; and
m is 0, 1, 2, or 3.

In some embodiments, the present disclosure provides a compound of Formula IIIB:

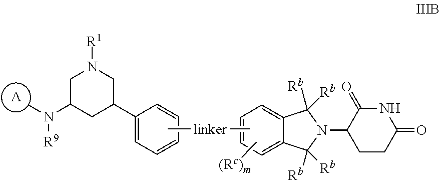

or a pharmaceutically acceptable salt thereof, wherein Ring A, R¹, R⁹, R^{b}, R^{c}, m, and linker are as defined above for Formula IIIA and described in classes and subclasses herein, both singly and in combination.

In some embodiments, the present disclosure provides a compound of Formula IIIB-1 or IIIB-2:

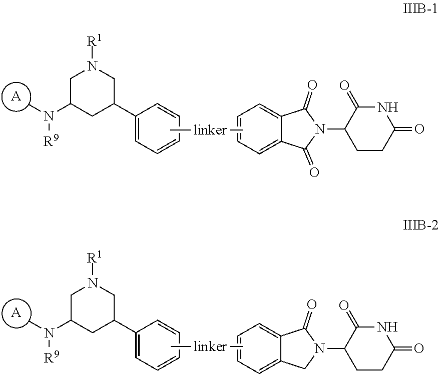

or a pharmaceutically acceptable salt thereof, wherein Ring A, R¹, R⁹, and linker are as defined above for Formula II and described in classes and subclasses herein, both singly and in combination. In some embodiments, the present disclosure provides a compound of Formula IIIB-1, or a pharmaceutically acceptable salt thereof. In some embodiments, the present disclosure provides a compound of Formula IIIB-2, or a pharmaceutically acceptable salt thereof.

In some embodiments, the present disclosure provides a compound of Formula IIIC:

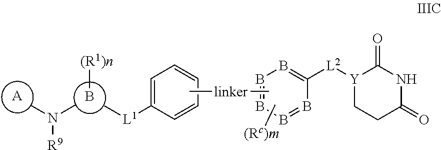

or a pharmaceutically acceptable salt thereof, wherein Ring A, Ring B, L¹, L², R¹, R⁹, Y, n, and linker are as defined above for Formula III and described in classes and subclasses herein, both singly and in combination;
and
each B is independently selected from N, C, and CH, provided that no more than two B are N;
each R^{c} is independently selected from halogen, —OR, —N(R)₂, —CN, and optionally substituted C₁₋₆ aliphatic; and
m is 0, 1, 2, or 3.

In some embodiments, the present disclosure provides a compound of Formula IIID:

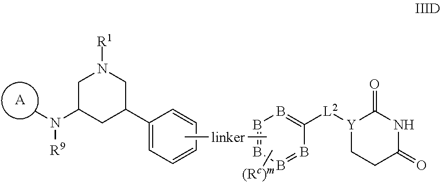

or a pharmaceutically acceptable salt thereof, wherein Ring A, B, L², R¹, R⁹, R^{c}, Y, m, and linker are as defined above for Formula IIIC and described in classes and subclasses herein, both singly and in combination.

In some embodiments, the present disclosure provides a compound of Formula IIID:

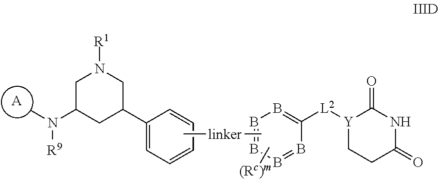

or a pharmaceutically acceptable salt, wherein:
Ring A is

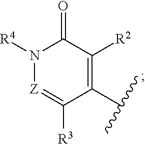

R¹ is optionally substituted C₁₋₆ aliphatic;
Z is N or CR³;
R² is hydrogen, halogen, —CN, optionally substituted C₁₋₆ aliphatic, or optionally substituted C₃₋₆ cycloaliphatic;
each R³ is independently hydrogen or halogen;
R⁴ is optionally substituted C₁₋₆ aliphatic;
R⁹ is hydrogen;
linker is an optionally substituted, bivalent, straight or branched, saturated or unsaturated C₁-C₁₀ hydrocarbon chain, wherein one or more methylene units are replaced by —O—, —N(R)—, —C(O)—, —OC(O)—, —C(O)O—, —C(O)N(R)—, —N(R)C(O)—, or -Cy-;
each Cy is independently an optionally substituted group selected from phenyl, 5- to 6-membered monocyclic heteroaryl having 1-4 heteroatoms independently selected from N, O, and S, monocyclic 4- to 7-membered heterocyclyl having 1-2 heteroatoms independently selected from N, O, and S, and bicyclic 6- to 11-membered heterocyclyl having 1-3 heteroatoms independently selected from N, O, and S;
each R is independently hydrogen or optionally substituted C₁₋₆ aliphatic;
L² is a covalent bond, —CH₂—, —O—, or —N(R)—;
Y is N or CH;
each B is independently selected from N, C, and CH, provided that no more than two B are N;
each R^{c} is independently selected from halogen, —OR, —N(R)₂, —CN, and optionally substituted C₁₋₆ aliphatic; and
m is 0, 1, 2, or 3.

In some embodiments, the present disclosure provides a compound of Formula IIID:

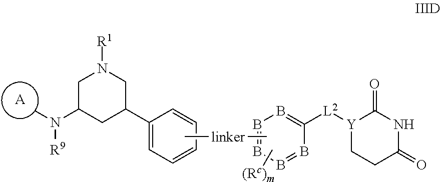

or a pharmaceutically acceptable salt, wherein:
Ring A is

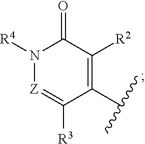

R¹ is C₁₋₆ alkyl;
Z is N or CR³;
R² is hydrogen, halogen, —CN, C₁₋₆ alkyl, or C₃₋₆ cycloalkyl;
each R³ is independently hydrogen or halogen;
R⁴ is C₁₋₆ alkyl;
R⁹ is hydrogen;
linker is

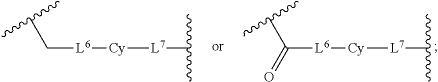

L⁶ and L⁷ are both a covalent bond;
Cy is is selected from:

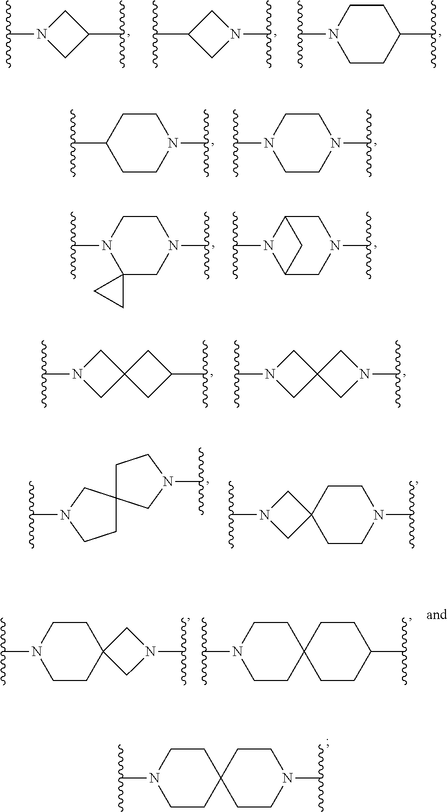

the moiety

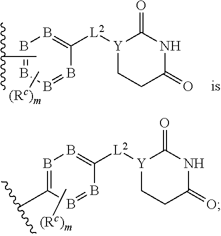

L² is a covalent bond;
Y is CH;
each B is independently selected from N, C, and CH, provided that no more than two B are N;
each R^{e} is independently selected from halogen, —O(C₁₋₆ alkyl), and C₁₋₆ alkyl; and
m is 0, 1, 2, or 3.

In some embodiments, the present disclosure provides a compound of Formula III-1:

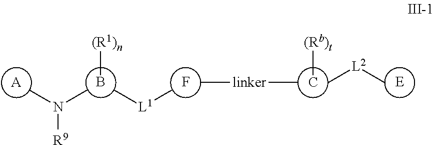

or a pharmaceutically acceptable salt thereof, wherein:
Ring A is selected from:

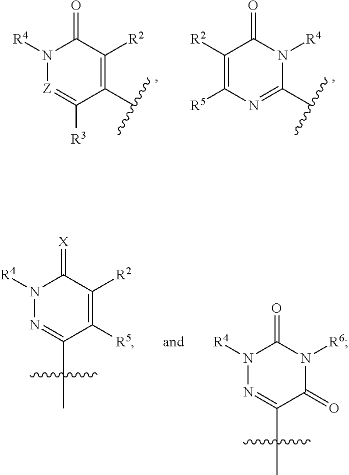

Ring B is an optionally substituted 5- to 6-membered heterocyclyl having 1-2 heteroatoms independently selected from N, O, and S;
L¹ is a covalent bond or an optionally substituted bivalent C₁₋₃ hydrocarbon chain;
each R¹ is independently —R;
n is 0, 1, 2, 3, or 4;
Z is N or CR³;
R² is halogen, —CN, or —R;
each R³ is independently halogen, —CN, or —R;
R⁴ is —R′;
R⁵ is halogen, —CN, or —R, or
   R² and R⁵, together with the atoms to which they are attached, combine to form an optionally substituted 5- to 6-membered ring having 0-2 heteroatoms independently selected from N, O, and S;
R⁶ is —R′;
X is O or NR⁷;
R⁷ is —R′, or
   R⁴ and R⁷, together with the atoms to which they are attached, combine to form an optionally substituted 5- to 6-membered ring having 2-3 heteroatoms independently selected from N, O, and S;
R⁹ is —R′;
linker is a covalent bond or an optionally substituted bivalent C₁-C₂₀ hydrocarbon chain, wherein one or more methylene units are optionally and independently replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —S(O)—, —SO₂—, —SO₂N(R)—, —N(R)SO₂—, or -Cy-;
each Cy is independently an optionally substituted, 3- to 16-membered bivalent ring having 0-6 heteroatoms independently selected from N, O, and S;
each of Ring C and Ring F is independently an optionally substituted 3- to 16-membered ring having 0-6 heteroatoms independently selected from N, O, and S;
each R^{b} is independently halogen, —CN, —R′ or —OR;
t is 0, 1, 2, 3, or 4;
L² is a covalent bond or an optionally substituted C₁₋₃ hydrocarbon chain, wherein one or more methylene units are optionally and independently replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —S(O)—, —SO₂—, —SO₂N(R)—, —N(R)SO₂—, or -Cy-;
Ring E is

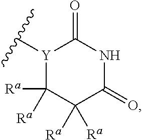

or an optionally substituted group selected from

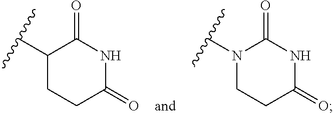 [SMILES: *C1CCC(=O)NC1=O ; *N1CCC(=O)NC1=O]

Y is N or CH;
each R^{a} is independently halogen, —CN or —R′;
each R′ is independently —R, —C(O)R, or —S(O)₂R, or two R′ attached to the same atom, together with the atom to which they are attached, combine to form an optionally substituted 3- to 16-membered ring having 1-5 heteroatoms independently selected from N, O, and S; and
each R is independently hydrogen or an optionally substituted group selected from C₁₋₈ aliphatic, C₃₋₁₀ cycloaliphatic, C₁-C₈ heteroaliphatic having 1-3 heteroatoms independently selected from N, O, and S, C₆₋₁₀ aryl, 5- to 10-membered heteroaryl having 1-5 heteroatoms independently selected from N, O, and S, and 3- to 10-membered monocyclic heterocyclyl having 1-5 heteroatoms independently selected from N, O, and S.

In some embodiments, the present disclosure provides a compound of Formula IIIE:

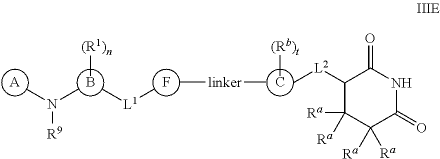

or a pharmaceutically acceptable salt thereof, wherein:
Ring A is selected from:

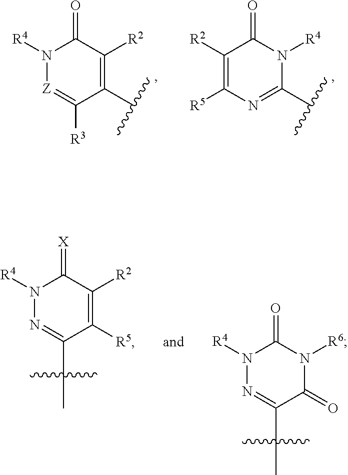

Ring B is an optionally substituted 5- to 6-membered heterocyclyl having 1-2 heteroatoms independently selected from N, O, and S;
L¹ is a covalent bond or an optionally substituted bivalent C₁₋₃ hydrocarbon chain;
each R¹ is independently —R;
n is 0, 1, 2, 3, or 4;
Z is N or CR³;
R² is halogen, —CN, or —R;
each R³ is independently halogen, —CN, or —R;
R⁴ is —R′;
R⁵ is halogen, —CN, or —R, or
   R² and R⁵, together with the atoms to which they are attached, combine to form an optionally substituted 5- to 6-membered ring having 0-2 heteroatoms independently selected from N, O, and S;
R⁶ is —R′;
X is O or NR⁷;
R⁷ is —R′, or
   R⁴ and R⁷, together with the atoms to which they are attached, combine to form an optionally substituted 5- to 6-membered ring having 2-3 heteroatoms independently selected from N, O, and S;
R⁹ is —R′;
linker is a covalent bond or an optionally substituted bivalent C₁-C₂₀ hydrocarbon chain, wherein one or more methylene units are optionally and independently replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —S(O)—, —SO₂—, —SO₂N(R)—, —N(R)SO₂—, or -Cy-;
each Cy is independently an optionally substituted, 3- to 16-membered bivalent ring having 0-6 heteroatoms independently selected from N, O, and S;
each of Ring C and Ring F is independently an optionally substituted 3- to 16-membered ring having 0-6 heteroatoms independently selected from N, O, and S;
each R^{b} is independently halogen, —CN, —R′ or —OR;
t is 0, 1, 2, 3, or 4;
L² is a covalent bond or an optionally substituted C₁₋₃ hydrocarbon chain, wherein one or more methylene units are optionally and independently replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—. —N(R)C(O)N(R)—. —N(R)C(O)O—, —OC(O)N(R)—, —N(R)C(O)S—, —SC(O)N(R)—, —N(R)C(NR)N(R)—, —S(O)—, —SO₂—, —SO₂N(R)—, —N(R)SO₂—, or -Cy-;
Y is N or CH;
each R^{a} is independently halogen, —CN or —R′;
each R′ is independently —R, —C(O)R, or —S(O)₂R, or two R′ attached to the same atom, together with the atom to which they are attached, combine to form an optionally substituted 3- to 16-membered ring having 1-5 heteroatoms independently selected from N, O, and S; and
each R is independently hydrogen or an optionally substituted group selected from C₁₋₈ aliphatic, C₃₋₁₀ cycloaliphatic, C₁-C₈ heteroaliphatic having 1-3 heteroatoms independently selected from N, O, and S, C₆₋₁₀ aryl, 5- to 10-membered heteroaryl having 1-5 heteroatoms independently selected from N, O, and S, and 3- to 10-membered monocyclic heterocyclyl having 1-5 heteroatoms independently selected from N, O, and S.

In some embodiments, the present disclosure provides a compound of Formula IIIF:

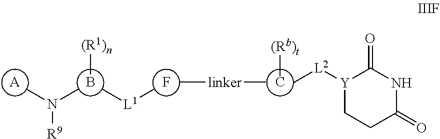

or a pharmaceutically acceptable salt thereof, wherein:
Ring A is selected from:

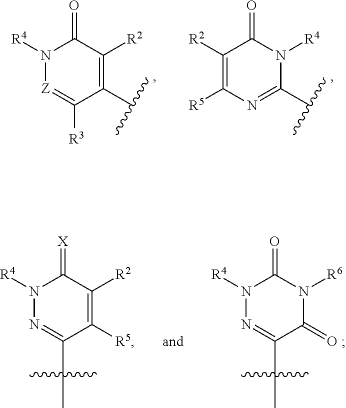

Ring B is an optionally substituted 5- to 6-membered heterocyclyl having 1-2 heteroatoms independently selected from N, O, and S;
L¹ is a covalent bond or an optionally substituted bivalent C₁₋₃ hydrocarbon chain;
each R¹ is independently —R;
n is 0, 1, 2, 3, or 4;
Z is N or CR³;
R² is halogen, —CN, or —R;
each R³ is independently halogen, —CN, or —R;
R⁴ is —R′;
R⁵ is halogen, —CN, or —R, or
   R² and R⁵, together with the atoms to which they are attached, combine to form an optionally substituted 5- to 6-membered ring having 0-2 heteroatoms independently selected from N, O, and S;
R⁶ is —R′;
X is O or NR⁷;
R⁷ is —R′, or
   R⁴ and R⁷, together with the atoms to which they are attached, combine to form an optionally substituted 5- to 6-membered ring having 2-3 heteroatoms independently selected from N, O, and S;
R⁹ is —R′;
linker is a covalent bond or an optionally substituted bivalent C₁-C₂₀ hydrocarbon chain, wherein one or more methylene units are optionally and independently replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —S(O)—, —SO₂—, —SO₂N(R)—, —N(R)SO₂—, or -Cy-;
each Cy is independently an optionally substituted, 3- to 16-membered bivalent ring having 0-6 heteroatoms independently selected from N, O, and S;
each of Ring C and Ring F is independently an optionally substituted 3- to 16-membered ring having 0-6 heteroatoms independently selected from N, O, and S;
each R^{b} is independently halogen, —CN, —R′ or —OR;
t is 0, 1, 2, 3, or 4;
L² is a covalent bond or an optionally substituted C₁₋₃ hydrocarbon chain, wherein one or more methylene units are optionally and independently replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —S(O)—, —SO₂—, —SO₂N(R)—, —N(R)SO₂—, or -Cy-;
Y is N or CH;
each R′ is independently —R, —C(O)R, or —S(O)₂R, or two R′ attached to the same atom, together with the atom to which they are attached, combine to form an optionally substituted 3- to 16-membered ring having 1-5 heteroatoms independently selected from N, O, and S; and
each R is independently hydrogen or an optionally substituted group selected from C₁₋₈ aliphatic, C₃₋₁₀ cycloaliphatic, C₁-C₈ heteroaliphatic having 1-3 heteroatoms independently selected from N, O, and S, C₆₋₁₀ aryl, 5- to 10-membered heteroaryl having 1-5 heteroatoms independently selected from N, O, and S, and 3- to 10-membered monocyclic heterocyclyl having 1-5 heteroatoms independently selected from N, O, and S.

In some embodiments, the present disclosure provides a compound of Formula IIIG:

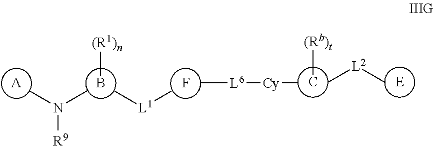

or a pharmaceutically acceptable salt thereof, wherein:
Ring A is selected from:

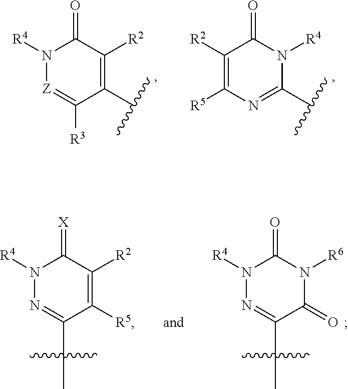

Ring B is an optionally substituted 5- to 6-membered heterocyclyl having 1-2 heteroatoms independently selected from N, O, and S;
L₁ is a covalent bond or an optionally substituted bivalent C₁₋₃ hydrocarbon chain;
each R¹ is independently —R;
n is 0, 1, 2, 3, or 4;
Z is N or CR³;
R² is halogen, —CN, or —R;
each R³ is independently halogen, —CN, or —R;
R⁴ is —R′;
R⁵ is halogen, —CN, or —R, or
   R² and R⁵, together with the atoms to which they are attached, combine to form an optionally substituted 5- to 6-membered ring having 0-2 heteroatoms independently selected from N, O, and S;
R⁶ is —R′;
X is O or NR⁷;
R⁷ is —R′, or
   R⁴ and R⁷, together with the atoms to which they are attached, combine to form an optionally substituted 5- to 6-membered ring having 2-3 heteroatoms independently selected from N, O, and S;
R⁹ is —R′;
L⁶ is a covalent bond, —C(O)— or optionally substituted —CH₂—;
Cy is an optionally substituted, 3- to 16-membered bivalent ring system having 0-6 heteroatoms independently selected from N, O, and S;
each of Ring C and Ring F is independently an optionally substituted 3- to 16-membered ring having 0-6 heteroatoms independently selected from N, O, and S;
each R^{b} is independently halogen, —CN, —R′ or —OR;
t is 0, 1, 2, 3, or 4;
L² is a covalent bond or an optionally substituted C₁₋₃ hydrocarbon chain, wherein one or more methylene units are optionally and independently replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —S(O)—, —SO₂—, —SO₂N(R)—, —N(R)SO₂—, or -Cy-;
Ring E is

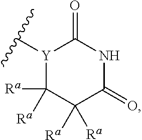

or an optionally substituted group selected from

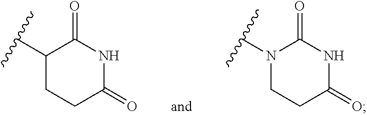 [SMILES: *C1CCC(=O)NC1=O ; *N1CCC(=O)NC1=O]

Y is N or CH;
each R^{a} is independently halogen, —CN or —R′;
each R′ is independently —R, —C(O)R, or —S(O)₂R, or two R′ attached to the same atom, together with the atom to which they are attached, combine to form an optionally substituted 3- to 16-membered ring having 1-5 heteroatoms independently selected from N, O, and S; and
each R is independently hydrogen or an optionally substituted group selected from C₁₋₈ aliphatic, C₃₋₁₀ cycloaliphatic, C₁-C₈ heteroaliphatic having 1-3 heteroatoms independently selected from N, O, and S, C₆₋₁₀ aryl, 5- to 10-membered heteroaryl having 1-5 heteroatoms independently selected from N, O, and S, and 3- to 10-membered monocyclic heterocyclyl having 1-5 heteroatoms independently selected from N, O, and S.

In some embodiments, the present disclosure provides a compound of Formula IIIH:

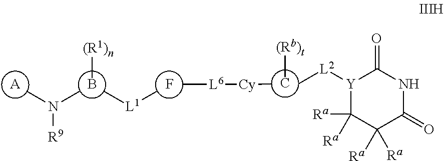

or a pharmaceutically acceptable salt thereof, wherein:
Ring A is selected from:

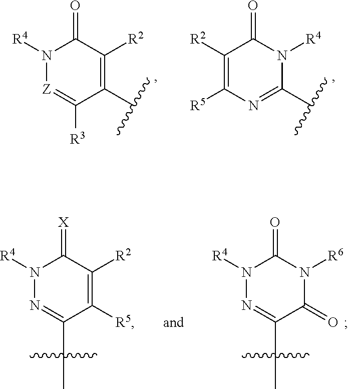

Ring B is an optionally substituted 5- to 6-membered heterocyclyl having 1-2 heteroatoms independently selected from N, O, and S;
L¹ is a covalent bond or an optionally substituted bivalent C₁₋₃ hydrocarbon chain;
each R¹ is independently —R;
n is 0, 1, 2, 3, or 4;
Z is N or CR³;
R² is halogen, —CN, or —R;
each R³ is independently halogen, —CN, or —R;
R⁴ is —R′;
R⁵ is halogen, —CN, or —R, or
   R² and R⁵, together with the atoms to which they are attached, combine to form an optionally substituted 5- to 6-membered ring having 0-2 heteroatoms independently selected from N, O, and S;
R⁶ is —R′;
X is O or NR⁷;
R⁷ is —R′, or
   R⁴ and R⁷, together with the atoms to which they are attached, combine to form an optionally substituted 5- to 6-membered ring having 2-3 heteroatoms independently selected from N, O, and S;
R⁹ is —R′;
L⁶ is a covalent bond, —C(O)— or optionally substituted —CH₂—;
Cy is an optionally substituted, 3- to 16-membered bivalent ring system having 0-6 heteroatoms independently selected from N, O, and S;
each of Ring C and Ring F is independently an optionally substituted 3- to 16-membered ring having 0-6 heteroatoms independently selected from N, O, and S;
each R^{b} is independently halogen, —CN, —R′ or —OR;
t is 0, 1, 2, 3, or 4;
L² is a covalent bond or an optionally substituted C₁₋₃ hydrocarbon chain, wherein one or more methylene units are optionally and independently replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —S(O)—, —SO₂—, —SO₂N(R)—, —N(R)SO₂—, or -Cy-;
Y is N or CH;
each R^{a} is independently halogen, —CN or —R′;
each R′ is independently —R, —C(O)R, or —S(O)₂R, or two R′ attached to the same atom, together with the atom to which they are attached, combine to form an optionally substituted 3- to 16-membered ring having 1-5 heteroatoms independently selected from N, O, and S; and
each R is independently hydrogen or an optionally substituted group selected from C₁₋₈ aliphatic, C₃₋₁₀ cycloaliphatic, C₁-C₈ heteroaliphatic having 1-3 heteroatoms independently selected from N, O, and S, C₆₋₁₀ aryl, 5- to 10-membered heteroaryl having 1-5 heteroatoms independently selected from N, O, and S, and 3- to 10-membered monocyclic heterocyclyl having 1-5 heteroatoms independently selected from N, O, and S.

In some embodiments, the present disclosure provides a compound of Formula IIIJ:

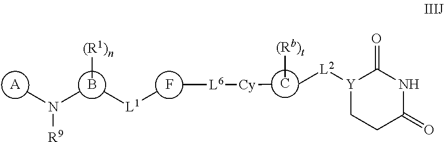

or a pharmaceutically acceptable salt thereof, wherein;
Ring A is selected from:

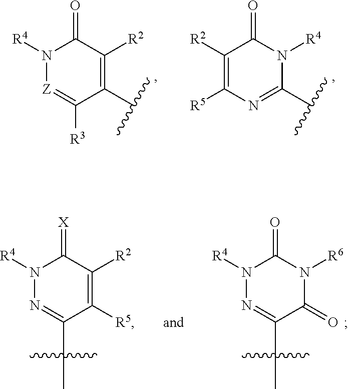

Ring B is an optionally substituted 5- to 6-membered heterocyclyl having 1-2 heteroatoms independently selected from N, O, and S;
L¹ is a covalent bond or an optionally substituted bivalent C₁₋₃ hydrocarbon chain;
each R¹ is independently —R;
n is 0, 1, 2, 3, or 4;
Z is N or CR³,
R² is halogen, —CN, or —R;
each R³ is independently halogen, —CN, or —R;
R⁴ is —R′;
R⁵ is halogen, —CN, or —R, or
   R² and R⁵, together with the atoms to which they are attached, combine to form an optionally substituted 5- to 6-membered ring baving 0-2 heteroatoms independently selected from N, O, and S;
R⁶ is —R′;
X is O or NR⁷;
R⁷ is —R′, or
   R⁴ and R⁷, together with the atoms to which they are attached, combine to form an optionally substituted 5- to 6-membered ring having 2-3 heteroatoms independently selected from N, O, and S;
R⁹ is —R′;
L⁶ is a covalent bond, —C(O)— or optionally substituted —CH₂—;
Cy is an optionally substituted, 3- to 16-membered bivalent ring system having 0-6 heteroatoms independently selected from N, O, and S;
each of Ring C and Ring F is independently an optionally substituted 3- to 16-membered ring having 0-6 heteroatoms independently selected from N, O, and S;
each R^{b} is independently halogen, —CN, —R′ or —OR;
t is 0, 1, 2, 3, or 4;
L² is a covalent bond or an optionally substituted C₁₋₃ hydrocarbon chain, wherein one or more methylenc units are optionally and independently replaced by —O—, —S—, ~N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —S(O)—, —SO₂—, —SO₂N(R)—, —N(R)SO₂—, or -Cy-;
Y is N or CH;
each R′ is independently —R, —C(O)R, or —S(O)₂R, or two R′ attached to the same atom, together with the atom to which they are attached, combine to form an optionally substituted 3- to 16-membered ring having 1-5 heteroatoms independently selected from N, O, and S; and
each R is independently hydrogen or an optionally substituted group selected from C₁₋₈ aliphatic, C₃₋₁₀ cycloaliphatic, C₁-C₈ heteroaliphatic having 1-3 heteroatoms independently selected from N, O, and S, C₆₋₁₀ aryl, 5- to 10-membered heteroaryl having 1-5 heteroatoms independently selected from N, O, and S, and 3- to 10-membered monocyclic heterocyclyl having 1-S heteroatoms independently selected from N, O, and S.

In some embodiments, the present disclosure provides a compound of Formula IIIK:

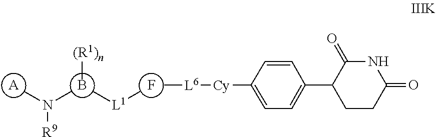

or a pharmaceutically acceptable salt thereof, wherein:
Ring A is selected from:

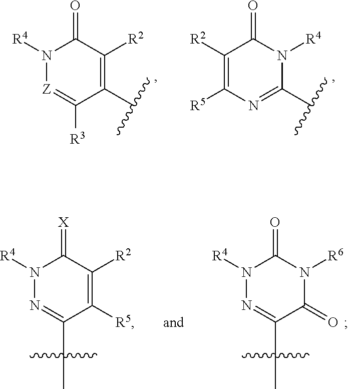

Ring B is an optionally substituted 5- to 6-membered heterocyclyl having 1-2 heteroatoms independently selected from N, O, and S;
L¹ is a covalent bond or an optionally substituted bivalent C₁₋₃ hydrocarbon chain;
each R¹ is independently —R;
n is 0, 1, 2, 3, or 4;
Z is N or CR³;
R² is halogen, —CN, or —R;
each R³ is independently halogen, —CN, or —R;
R⁴ is —R′;
R⁵ is halogen, —CN, or —R, or
   R² and R⁵, together with the atoms to which they are attached, combine to form an optionally substituted 5- to 6-membered ring having 0-2 heteroatoms independently selected from N, O, and S;
R⁶ is —R′;
X is O or NR⁷;
R⁷ is —R′, or
   R⁴ and R⁷, together with the atoms to which they are attached, combine to form an optionally substituted 5- to 6-membered ring having 2-3 heteroatoms independently selected from N, O, and S;
R⁹ is —R′;
L⁶ is a covalent bond, —C(O)— or optionally substituted —CH₂—;
Cy is an optionally substituted, 3- to 16-membered bivalent ring system having 0-6 heteroatoms independently selected from N, O, and S;
Ring F is an optionally substituted 3- to 16-membered ring having 0-6 heteroatoms independently selected from N, O, and S;
L² is a covalent bond or an optionally substituted C₁₋₃ hydrocarbon chain, wherein one or more methylene units are optionally and independently replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —S(O)—, —SO₂—, —SO₂N(R)—, —N(R)SO₂—, or -Cy-;
each R′ is independently —R, —C(O)R, or —S(O)₂R, or two R′ attached to the same atom, together with the atom to which they are attached, combine to form an optionally substituted 3- to 16-membered ring having 1-5 heteroatoms independently selected from N, O, and S; and
each R is independently hydrogen or an optionally substituted group selected from C₁₋₈ aliphatic, C₃₋₁₀ cycloaliphatic, C₁-C₈ heteroaliphatic having 1-3 heteroatoms independently selected from N, O, and S, C₆₋₁₀ aryl, 5- to 10-membered heteroaryl having 1-5 heteroatoms independently selected from N, O, and S, and 3- to 10-membered monocyclic heterocyclyl having 1-5 heteroatoms independently selected from N, O, and S.

In some embodiments, the present disclosure provides a compound of Formula IIIL:

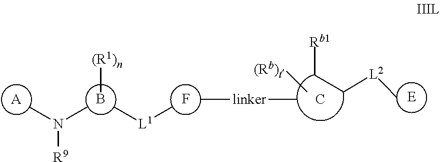

or a pharmaceutically acceptable salt thereof, wherein;
Ring A is selected from:

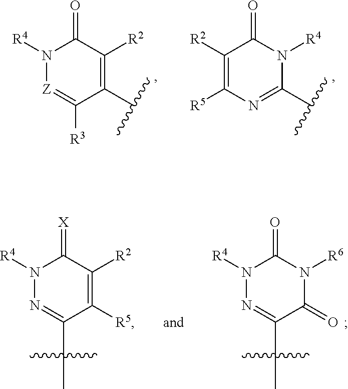

Ring B is an optionally substituted 5- to 6-membered heterocyclyl having 1-2 heteroatoms independently selected from N, O, and S;
L¹ is a covalent bond or an optionally substituted bivalent C₁₋₃ hydrocarbon chain;
each R¹ is independently —R;
n is 0, 1, 2, 3, or 4;
Z is N or CR³;
R² is halogen, —CN, or —R;
each R³ is independently halogen, —CN, or —R;
R⁴ is —R′;
R⁵ is halogen, —CN, or —R, or
   R² and R⁵, together with the atoms to which they are attached, combine to form an optionally substituted 5- to 6-membered ring having 0-2 heteroatoms independently selected from N, O, and S;
R⁶ is —R′;
X is O or NR⁷;
R⁷ is —R′, or
   R⁴ and R⁷, together with the atoms to which they are attached, combine to form an optionally substituted 5- to 6-membered ring having 2-3 heteroatoms independently selected from N, O, and S;
R⁹ is —R′;
linker is a covalent bond or an optionally substituted bivalent C₁-C₂₀ hydrocarbon chain, wherein one or more methylene units are optionally and independently replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —SC(O)N(R)—, —N(R)C(NR)N(R)—, —S(O)—, —SO₂—, —SO₂N(R)—, —N(R)SO₂—, or -Cy-;
each Cy is independently an optionally substituted, 3- to 16-membered bivalent ring having 0-6 heteroatoms independently selected from N, O, and S;
each of Ring C and Ring F is independently an optionally substituted 3- to 16-membered ring having 0-6 heteroatoms independently selected from N, O, and S;
each R^{b} and R^{b1} is independently halogen, —CN, —R′ or —OR;
t′ is 0, 1, 2, or 3;
L² is a covalent bond or an optionally substituted C₁₋₃ hydrocarbon chain, wherein one or more methylene units are optionally and independently replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —S(O)—, —SO₂—, —SO₂N(R)—, —N(R)SO₂—, or -Cy-;
Ring E is

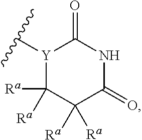

or an optionally substituted group selected from

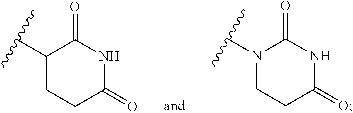 [SMILES: *C1CCC(=O)NC1=O ; "and" ; *N1CCC(=O)NC1=O]

Y is N or CH;
each R^{a} is independently halogen, —CN or —R′;
each R′ is independently —R, —C(O)R, or —S(O)₂R, or two R′ attached to the same atom, together with the atom to which they are attached, combine to form an optionally substituted 3- to 16-membered ring having 1-5 heteroatoms independently selected from N, O, and S; and
each R is independently hydrogen or an optionally substituted group selected from C₁₋₈ aliphatic, C₃₋₁₀ cycloaliphatic, C₁-C₈ heteroaliphatic having 1-3 heteroatoms independently selected from N, O, and S, C₆₋₁₀ aryl, 5- to 10-membered heteroaryl having 1-5 heteroatoms independently selected from N, O, and S, and 3- to 10-membered monocyclic heterocyclyl having 1-5 heteroatoms independently selected from N, O, and S.

In some embodiments, the present disclosure provides a compound of Formula IIIM:

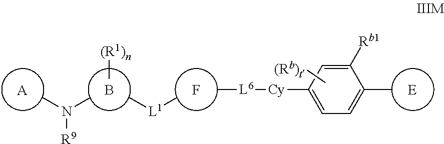

or a pharmaceutically acceptable salt thereof, wherein:
Ring A is selected from:

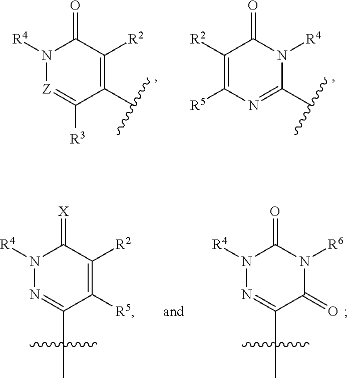

Ring B is an optionally substituted 5- to 6-membered heterocyclyl having 1-2 heteroatoms independently selected from N, O, and S;
L¹ is a covalent bond or an optionally substituted bivalent C₁₋₃ hydrocarbon chain;
each R′ is independently —R;
n is 0, 1, 2, 3, or 4;
Z is N or CR³;
R² is halogen, —CN, or —R;
each R³ is independently halogen, —CN, or —R;
R⁴ is —R′;
R⁵ is halogen, —CN, or —R, or
   R² and R⁵, together with the atoms to which they are attached, combine to form an optionally substituted 5- to 6-membered ring having 0-2 heteroatoms independently selected from N, O, and S;
R⁶ is —R′;
X is O or NR⁷;
R⁷ is —R′, or
   R⁴ and R⁷, together with the atoms to which they are attached, combine to form an optionally substituted 5- to 6-membered ring having 2-3 heteroatoms independently selected from N, O, and S;
R⁹ is —R′;
L⁶ is a covalent bond, —C(O)— or optionally substituted —CH₂—;
Cy is an optionally substituted, 3- to 16-membered bivalent ring system having 0-6 heteroatoms independently selected from N, O, and S;
Ring F is an optionally substituted 3- to 16-membered ring having 0-6 heteroatoms independently selected from N, O, and S;
each R^{b} and R^{b1} is independently halogen, —CN, —R′ or —OR;
t′ is 0, 1, 2, or 3;
L² is a covalent bond or an optionally substituted C₁₋₃ hydrocarbon chain, wherein one or more methylene units are optionally and independently replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —S(O)—, —SO₂—, —SO₂N(R)—, —N(R)SO₂—, or -Cy-;
Ring E is

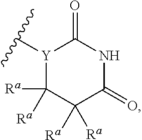

or an optionally substituted group selected from

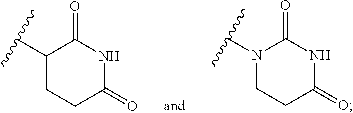 [SMILES: *C1CCC(=O)NC1=O ; "and" ; *N1CCC(=O)NC1=O]

Y is N or CH;
each R^{a} is independently halogen, —CN or —R′;
each R′ is independently —R, —C(O)R, or —S(O)₂R, or two R′ attached to the same atom, together with the atom to which they are attached, combine to form an optionally substituted 3- to 16-membered ring having 1-5 heteroatoms independently selected from N, O, and S; and
each R is independently hydrogen or an optionally substituted group selected from C₁₋₈ aliphatic, C₃₋₁₀ cycloaliphatic, C₁-C₈ heteroaliphatic having 1-3 heteroatoms independently selected from N, O, and S, C₆₋₁₀ aryl, 5- to 10-membered heteroaryl having 1-5 heteroatoms independently selected from N, O, and S, and 3- to 10-membered monocyclic heterocyclyl having 1-5 heteroatoms independently selected from N, O, and S.

In some embodiments, the present disclosure provides a compound of Formula IIIN:

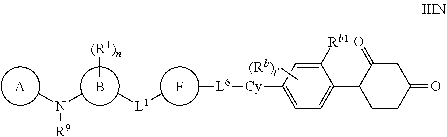

or a pharmaceutically acceptable salt thereof, wherein:
Ring A is selected from:

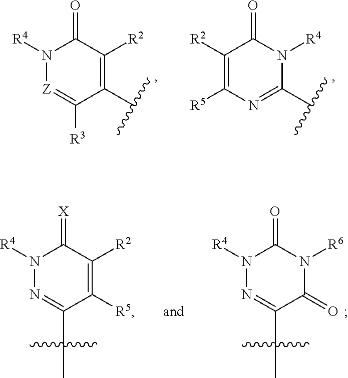

Ring B is an optionally substituted 5- to 6-membered heterocyclyl having 1-2 heteroatoms independently selected from N, O, and S;
L¹ is a covalent bond or an optionally substituted bivalent C₁₋₃ hydrocarbon chain;
each R¹ is independently —R;
n is 0, 1, 2, 3, or 4;
Z is N or CR³;
R² is halogen, —CN, or —R;
each R³ is independently halogen, —CN, or —R;
R⁴ is —R′;
R⁵ is halogen, —CN, or —R, or
   R² and R⁵, together with the atoms to which they are attached, combine to form an optionally substituted 5- to 6-membered ring having 0-2 heteroatoms independently selected from N, O, and S;
R⁶ is —R′;
X is O or NR⁷;
R⁷ is —R′, or
   R⁴ and R⁷, together with the atoms to which they are attached, combine to form an optionally substituted 5- to 6-membered ring having 2-3 heteroatoms independently selected from N, O, and S;
R⁹ is —R′;
L⁶ is a covalent bond, —C(O)— or optionally substituted —CH₂—;
Cy is an optionally substituted, 3- to 16-membered bivalent ring system having 0-6 heteroatoms independently selected from N, O, and S;
Ring F is an optionally substituted 3- to 16-membered ring having 0-6 heteroatoms independently selected from N, O, and S;
each R^{b} and R^{b1} is independently halogen, —CN, —R′ or —OR;
t′ is 0, 1, 2, or 3;
L² is a covalent bond or an optionally substituted C₁₋₃ hydrocarbon chain, wherein one or more methylene units are optionally and independently replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —S(O)—, —SO₂—, —SO₂N(R)—, —N(R)SO₂—, or -Cy-;
Y is N or CH;
each R^{a} is independently halogen, —CN or —R′;
each R′ is independently —R, —C(O)R, or —S(O)₂R, or two R′ attached to the same atom, together with the atom to which they are attached, combine to form an optionally substituted 3- to 16-membered ring having 1-5 heteroatoms independently selected from N, O, and S; and
each R is independently hydrogen or an optionally substituted group selected from C₁₋₈ aliphatic, C₃₋₁₀ cycloaliphatic, C₁-C₈ heteroaliphatic having 1-3 heteroatoms independently selected from N, O, and S, C₆₋₁₀ aryl, 5- to 10-membered heteroaryl having 1-5 heteroatoms independently selected from N, O, and S, and 3- to 10-membered monocyclic heterocyclyl having 1-5 heteroatoms independently selected from N, O, and S.

In some embodiments, the present disclosure provides a compound of Formula IV:

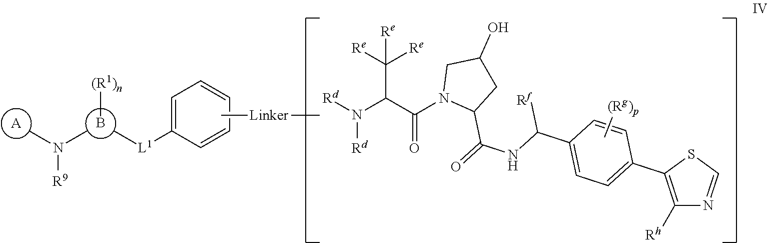

or a pharmaceutically acceptable salt thereof, wherein Ring A, Ring B, L¹, R¹, R⁹, n, and linker are as defined above for Formula II and described in classes and subclasses herein, both singly and in combination; and
linker is attached to the bracketed moiety at one of R^{d}, R^{e}, R^{f}, or R^{g};
each R^{d} that is not the point of attachment for the linker is independently hydrogen, —C(O)R, or optionally substituted C₁₋₆ aliphatic, or
   two R^{d}, together with the atom to which they are attached, combine to form an optionally substituted 5- to 6-membered ring having 1-3 heteroatoms independently selected from N, O, and S and optionally fused to a phenyl or 5- to 6-membered heteroaryl ring having 1-3 heteroatoms independently selected from N, O, and S;
each R^{e} that is not the point of attachment for the linker is independently hydrogen or optionally substituted C₁₋₆ aliphatic;
R^{f}, when it is not the point of attachment for the linker, is hydrogen or optionally substituted C₁₋₆ aliphatic;
each R^{g} that is not the point of attachment for the linker is independently halogen, —OR, —CN, or optionally substituted C₁₋₆ aliphatic;
R^{h} is hydrogen, halogen, or optionally substituted C₁₋₆ aliphatic; and
p is 0, 1, 2, or 3.

In some embodiments, the present disclosure provides a compound of Formula IVA:

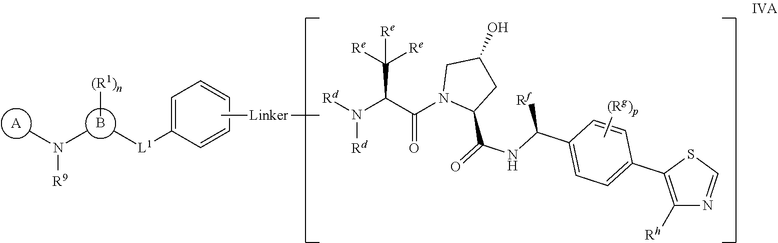

or a pharmaceutically acceptable salt thereof, wherein Ring A, Ring B, L¹, R¹, R⁹, R^{d}, R^{e}, R^{f}, R^{g}, R^{h}, p, n, and linker are as defined above for Formula IV and described in classes and subclasses herein, both singly and in combination.

In some embodiments, the present disclosure provides a compound of Formula IVA-1:

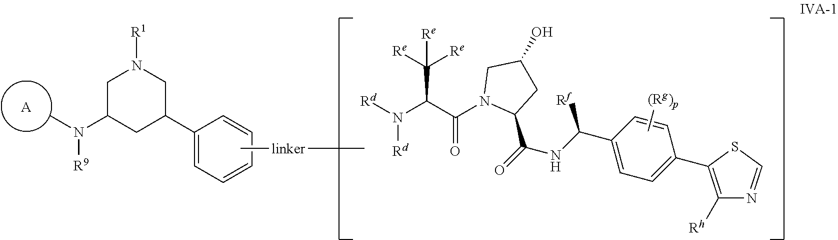

or a pharmaceutically acceptable salt thereof, wherein Ring A, R¹, R⁹, R^{d}, R^{e}, R^{f}, R^{g}, R^{h}, p, and linker are as defined above for Formula IV and described in classes and subclasses herein, both singly and in combination.

In some embodiments, the present disclosure provides a compound of Formula IV-1:

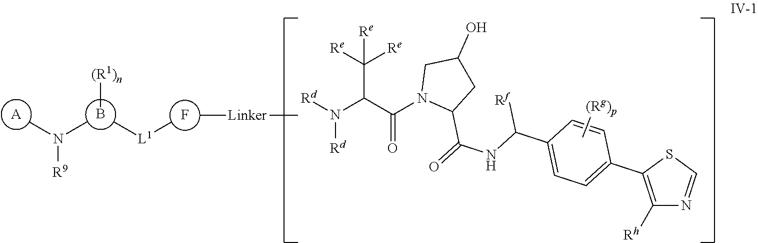

or a pharmaceutically acceptable salt thereof, wherein:
Ring A is selected from:

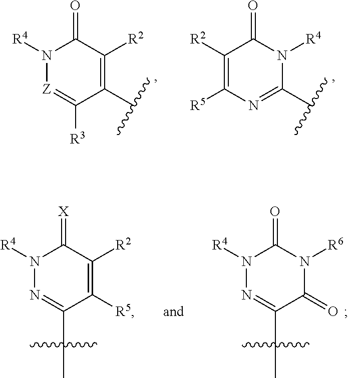

Ring B is an optionally substituted 5- to 6-membered heterocyclyl having 1-2 heteroatoms independently selected from N, O, and S;
L¹ is a covalent bond or an optionally substituted bivalent C₁₋₃ hydrocarbon chain;
each R¹ is independently —R;
n is 0, 1, 2, 3, or 4;
Z is N or CR³;
R² is halogen, —CN, or —R;
each R³ is independently halogen, —CN, or —R;
R⁴ is —R′;
R⁵ is halogen, —CN, or —R, or
   R² and R⁵, together with the atoms to which they are attached, combine to form an optionally substituted 5- to 6-membered ring having 0-2 heteroatoms independently selected from N, O, and S;
R⁶ is —R′;
X is O or NR⁷;
R⁷ is —R′, or
   R⁴ and R⁷, together with the atoms to which they are attached, combine to form an optionally substituted 5- to 6-membered ring having 2-3 heteroatoms independently selected from N, O, and S;
R⁹ is —R′;
linker is a covalent bond or an optionally substituted bivalent C₁-C₂₀ hydrocarbon chain, wherein one or more methylene units are optionally and independently replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —S(O)—, —SO₂—, —SO₂N(R)—, —N(R)SO₂—, or -Cy-;
each Cy is independently an optionally substituted, 3- to 16-membered bivalent ring having 0-6 heteroatoms independently selected from N, O, and S;
Ring F is an optionally substituted 3- to 16-membered ring having 0-6 heteroatoms independently selected from N, O, and S;
linker is attached to the bracketed moiety at one of R^{d}, R^{e}, R^{f}, or R^{g};
each R^{d} that is not the point of attachment for the linker is independently —R′, or
   two R^{d}, together with the atom to which they are attached, combine to form an optionally substituted 5- to 6-membered ring having 1-3 heteroatoms independently selected from N, O, and S and optionally fused to a phenyl or 5- to 6-membered heteroaryl ring having 1-3 heteroatoms independently selected from N, O, and S;
each R^{e} that is not the point of attachment for the linker is independently —R;
R^{f}, when it is not the point of attachment for the linker, is —R;
each R^{g} that is not the point of attachment for the linker is independently halogen, —CN, R or —OR;
p is 0, 1, 2, or 3;
R^{h} is halogen, or —R;
each R′ is independently —R, —C(O)R, or —S(O)₂R, or two R′ attached to the same atom, together with the atom to which they are attached, combine to form an optionally substituted 3- to 16-membered ring having 1-5 heteroatoms independently selected from N, O, and S; and
each R is independently hydrogen or an optionally substituted group selected from C₁₋₈ aliphatic, C₃₋₁₀ cycloaliphatic, C₁-C₈ heteroaliphatic having 1-3 heteroatoms independently selected from N, O, and S, C₆₋₁₀ aryl, 5- to 10-membered heteroaryl having 1-5 heteroatoms independently selected from N, O, and S, and 3- to 10-membered monocyclic heterocyclyl having 1-5 heteroatoms independently selected from N, O, and S.

In some embodiments, a compound of formula IV-1 is a compound of formula IVD:

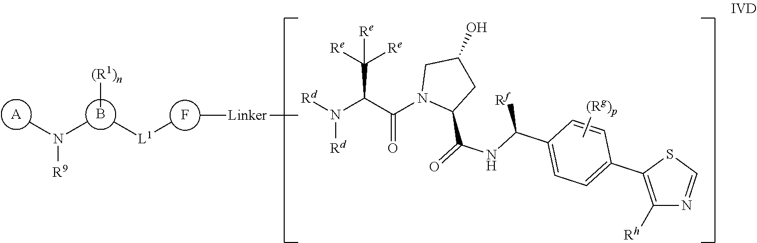

wherein the variables are as defined above for Formula IVB and described in classes and subclasses herein, both singly and in combination.

In some embodiments, the present disclosure provides a compound of Formula V:

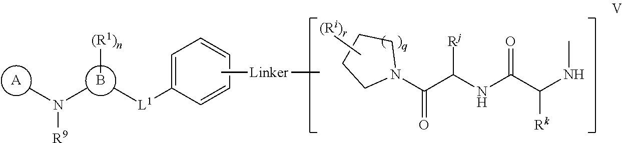

or a pharmaceutically acceptable salt thereof, wherein Ring A, Ring B, L¹, R¹, R⁹, n, and linker are as defined above for Formula II and described in classes and subclasses herein, both singly and in combination; and
linker is attached to the bracketed moiety at one Rⁱ or the ring formed when two Rⁱ groups are taken together;
each Rⁱ that is not the point of attachment of the linker is independently halogen, optionally substituted C₁₋₆ aliphatic, —C(O)N(R)₂, or —N(R)C(O)R, or
   two Rⁱ groups, together with the atoms to which they are attached, combine to form an optionally substituted phenyl or 5- to 6-membered heteroaryl ring having 1-3 heteroatoms independently selected from N, O, and S;
R^{j} is an optionally substituted group selected from C₁₋₆ aliphatic and C₃₋₇ cycloaliphatic, or
   R^{j} combines with one instance of Rⁱ, together with the atoms to which they are attached, to form an optionally substituted 5- to 7-membered heterocycle having 1-2 heteroatoms independently selected from N, O, and S;
R^{k} is an optionally substituted C₁₋₆ aliphatic;
r is 1, 2, 3, 4, or 5; and
q is 1 or 2.

In some embodiments, the present disclosure provides a compound of Formula VA:

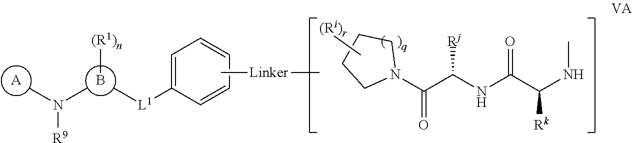

or a pharmaceutically acceptable salt thereof, wherein Ring A, Ring B, L¹, R¹, R⁹, Rⁱ, R^{j}, R^{k}, n, q, r, and linker are as defined above for Formula V and described in classes and subclasses herein, both singly and in combination.

In some embodiments, the present disclosure provides a compound of Formula VA-1:

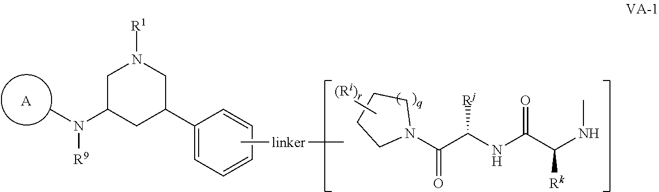

or a pharmaceutically acceptable salt thereof, wherein Ring A, R¹, R⁹, Rⁱ, R^{j}, R^{k}, q, r, and linker are as defined above for Formula V and described in classes and subclasses herein, both singly and in combination.

In some embodiments, the present disclosure provides a compound of Formula VB:

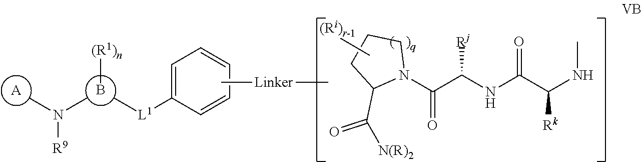

or a pharmaceutically acceptable salt thereof, wherein Ring A, Ring B, L¹, R, R¹, R⁹, Rⁱ, R^{j}, R^{k}, n, q, r, and linker are as defined above for Formula V and described in classes and subclasses herein, both singly and in combination.

In some embodiments, the present disclosure provides a compound of Formula VB-1:

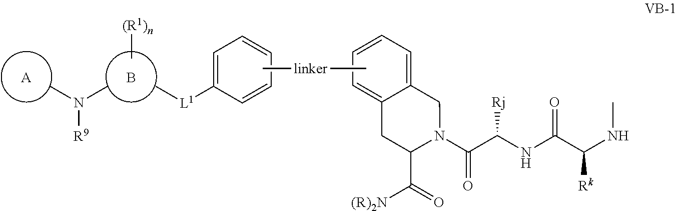

or a pharmaceutically acceptable salt thereof, wherein Ring A, Ring B, L¹, R, R¹, R⁹, R^{j}, R^{k}, n, and linker are as defined above for Formula V and described in classes and subclasses herein, both singly and in combination.

In some embodiments, the present disclosure provides a compound of Formula V-1:

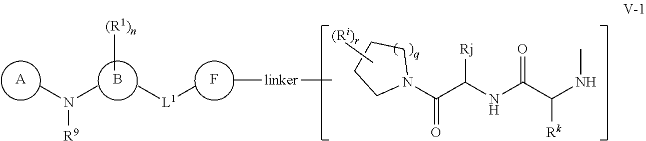

or a pharmaceutically acceptable salt thereof, wherein:
Ring A is selected from:

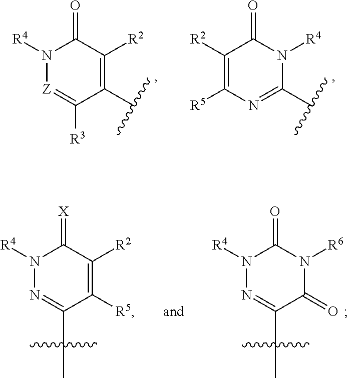

Ring B is an optionally substituted 5- to 6-membered heterocyclyl having 1-2 heteroatoms independently selected from N, O, and S;
L¹ is a covalent bond or an optionally substituted bivalent C₁₋₃ hydrocarbon chain;
each R¹ is independently —R;
n is 0, 1, 2, 3, or 4;
Z is N or CR³;
R² is halogen, —CN, or —R;
each R³ is independently halogen, —CN, or —R;
R⁴ is —R′;
R⁵ is halogen, —CN, or —R, or
   R² and R⁵, together with the atoms to which they are attached, combine to form an optionally substituted 5- to 6-membered ring having 0-2 heteroatoms independently selected from N, O, and S;
R⁶ is —R′;
X is O or NR⁷;
R⁷ is —R′, or
   R⁴ and R⁷, together with the atoms to which they are attached, combine to form an optionally substituted 5- to 6-membered ring having 2-3 heteroatoms independently selected from N, O, and S;
R⁹ is —R′;
linker is a covalent bond or an optionally substituted bivalent C₁-C₂₀ hydrocarbon chain, wherein one or more methylene units are optionally and independently replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —S(O)—, —SO₂—, —SO₂N(R)—, —N(R)SO₂—, or -Cy-;
each Cy is independently an optionally substituted, 3- to 16-membered bivalent ring having 0-6 heteroatoms independently selected from N, O, and S;
Ring F is an optionally substituted 3- to 16-membered ring having 0-6 heteroatoms independently selected from N, O, and S;
linker is attached to the bracketed moiety at one Rⁱ or the ring formed when two Rⁱ groups are taken together;
each Rⁱ that is not the point of attachment of the linker is independently halogen, —R, —C(O)N(R′)₂, or —N(R)C(O)R, or
   two Rⁱ groups, together with the atoms to which they are attached, combine to form an optionally substituted phenyl or 5- to 6-membered ring having 1-3 heteroatoms independently selected from N, O, and S;
R^{j} is —R, or
   R^{j} combines with one instance of Rⁱ, together with the atoms to which they are attached, to form an optionally substituted 5- to 7-membered ring having 1-2 heteroatoms independently selected from N, O, and S;
R^{k} is —R;
r is 1, 2, 3, 4, or 5;
q is 1 or 2,
each R′ is independently —R, —C(O)R, or —S(O)₂R, or two R′ attached to the same atom, together with the atom to which they are attached, combine to form an optionally substituted 3- to 16-membered ring having 1-5 heteroatoms independently selected from N, O, and S; and each R is independently hydrogen or an optionally substituted group selected from Cis aliphatic, C₃₋₁₀ cycloaliphatic, C₁-C₈ heteroaliphatic having 1-3 heteroatoms independently selected from N, O, and S, C₆₋₁₀ aryl, 5- to 10-membered heteroaryl having 1-5 heteroatoms independently selected from N, O, and S, and 3- to 10-membered monocyclic heterocyclyl having 1-5 heteroatoms independently selected from N, O, and S.

In some embodiments, a compound of formula V-1 is of formula VC:

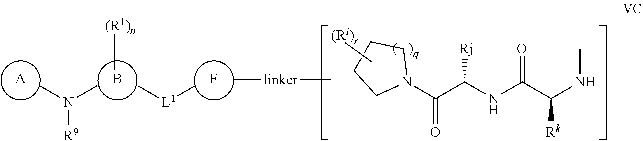

wherein the variables are as defined above for Formula V-1 and described in classes and subclasses herein, both singly and in combination.

In some embodiments, a compound of formula V-1 is of formula VD:

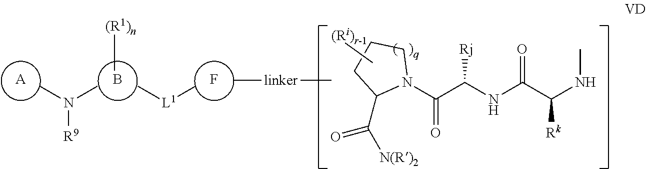

wherein the variables are as defined above for Formula V-1 and described in classes and subclasses herein, both singly and in combination.

In some embodiments, a compound of formula V-1 is of formula VE:

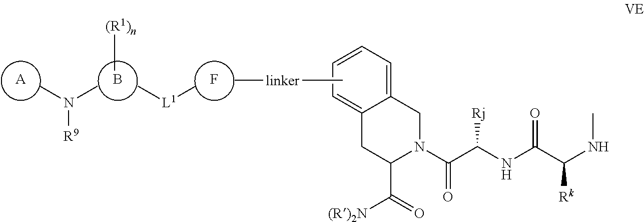

wherein the variables are as defined above for Formula V-1 and described in classes and subclasses herein, both singly and in combination.

In some embodiments, the present disclosure provides a compound of Formula VI:

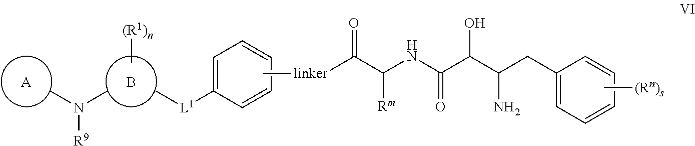

or a pharmaceutically acceptable salt thereof, wherein Ring A, Ring B, L¹, R¹, R⁹, n, and linker are as defined above for Formula II and described in classes and subclasses herein, both singly and in combination; and
R^{m} is optionally substituted C₁₋₆ aliphatic;
each Rⁿ is independently halogen, —OR, —CN, or optionally substituted C₁₋₆ aliphatic; and
s is 0, 1, 2, 3, 4, or 5.

In some embodiments, the present disclosure provides a compound of Formula VIA:

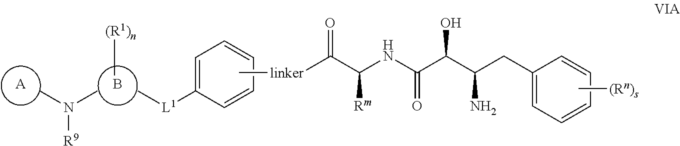

or a pharmaceutically acceptable salt thereof, wherein Ring A, Ring B, L¹, R¹, R⁹, R^{m}, Rⁿ, n, s, and linker are as defined above for Formula VI and described in classes and subclasses herein, both singly and in combination.

In some embodiments, the present disclosure provides a compound of Formula VIA-1:

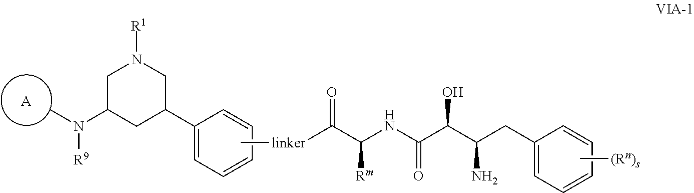

or a pharmaceutically acceptable salt thereof, wherein Ring A, R¹, R⁹, R^{m}, Rⁿ, s, and linker are as defined above for Formula VI and described in classes and subclasses herein, both singly and in combination.
In some embodiments, the present disclosure provides a compound of Formula VI-1:

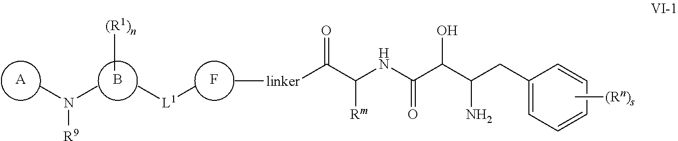

or a pharmaceutically acceptable salt thereof, wherein:
Ring A is selected from:

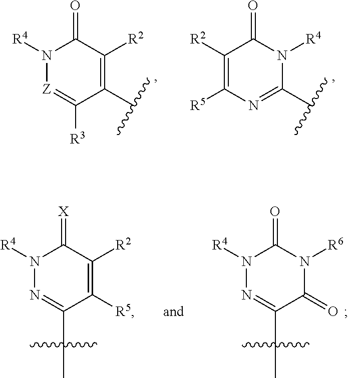

Ring B is an optionally substituted 5- to 6-membered heterocyclyl having 1-2 heteroatoms independently selected from N, O, and S;
L¹ is a covalent bond or an optionally substituted bivalent C₁₋₃ hydrocarbon chain;
each R¹ is independently —R;
n is 0, 1, 2, 3, or 4;
Z is N or CR³;
R² is halogen, —CN, or —R;
each R³ is independently halogen, —CN, or —R;
R⁴ is —R′;
R⁵ is halogen, —CN, or —R, or
   R² and R⁵, together with the atoms to which they are attached, combine to form an optionally substituted 5- to 6-membered ring having 0-2 heteroatoms independently selected from N, O, and S;
R⁶ is —R′;
X is O or NR⁷;
R⁷ is —R′, or
   R⁴ and R⁷, together with the atoms to which they are attached, combine to form an optionally substituted 5- to 6-membered ring having 2-3 heteroatoms independently selected from N, O, and S;
R⁹ is —R′;
linker is a covalent bond or an optionally substituted bivalent C₁-C₂₀ hydrocarbon chain, wherein one or more methylene units are optionally and independently replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —S(O)—, —SO₂—, —SO₂N(R)—, —N(R)SO₂—, or -Cy-;
each Cy is independently an optionally substituted, 3- to 16-membered bivalent ring having 0-6 heteroatoms independently selected from N, O, and S;
Ring F is an optionally substituted 3- to 16-membered ring having 0-6 heteroatoms independently selected from N, O, and S;
R^{m} is R;
each Rⁿ is independently halogen, —CN, —R or —OR;
s is 0, 1, 2, 3, 4, or 5;
each R′ is independently —R, —C(O)R, or —S(O)₂R, or two R′ attached to the same atom, together with the atom to which they are attached, combine to form an optionally substituted 3- to 16-membered ring having 1-5 heteroatoms independently selected from N, O, and S; and
each R is independently hydrogen or an optionally substituted group selected from C₁₋₈ aliphatic, C₃₋₁₀ cycloaliphatic, C₁-C₈ heteroaliphatic having 1-3 heteroatoms independently selected from N, O, and S, C₆₋₁₀ aryl, 5- to 10-membered heteroaryl having 1-5 heteroatoms independently selected from N, O, and S, and 3- to 10-membered monocyclic heterocyclyl having 1-5 heteroatoms independently selected from N, O, and S.

In some embodiments, the present disclosure provides a compound of Formula VIB:

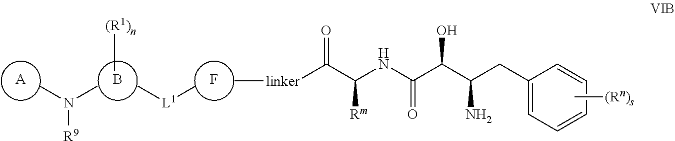

or a pharmaceutically acceptable salt thereof, wherein the variables are as defined above for Formula VI-1 and described in classes and subclasses herein, both singly and in combination.

In some embodiments, the present disclosure provides a compound of Formula VII:

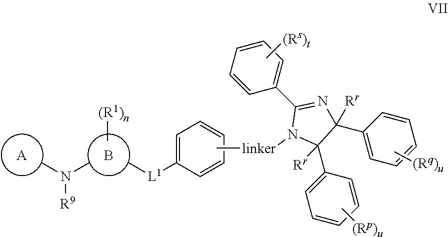

or a pharmaceutically acceptable salt thereof, wherein Ring A, Ring B, L¹, R¹, R⁹, n, and linker are as defined above for Formula II and described in classes and subclasses herein, both singly and in combination; and
each R^{p} is independently halogen, —OR, —CN, or optionally substituted C₁₋₆ aliphatic;
each R^{q} is independently halogen, —OR, —CN, or optionally substituted C₁₋₆ aliphatic;
each R^{r} is independently hydrogen or optionally substituted C₁₋₆ aliphatic;
each R^{s} is independently halogen, —OR, —CN, or optionally substituted C₁₋₆ aliphatic;
t is 0, 1, 2, 3, 4, or 5; and
each u is independently 0, 1, 2, 3, 4, or 5.

In some embodiments, the present disclosure provides a compound of Formula VIIA:

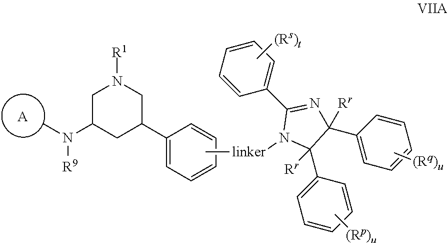

or a pharmaceutically acceptable salt thereof, wherein Ring A, R¹, R⁹, R^{p}, R^{q}, R^{r}, R^{s}, u, t, and linker are as defined above for Formula VII and described in classes and subclasses herein, both singly and in combination.

In some embodiments, the present disclosure provides a compound of Formula VII-1:

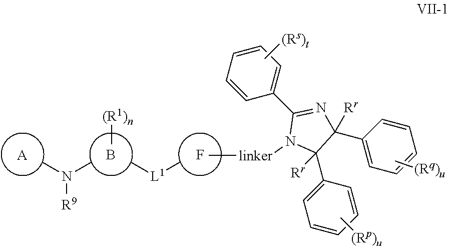

or a pharmaceutically acceptable salt thereof,
wherein Ring A is selected from:

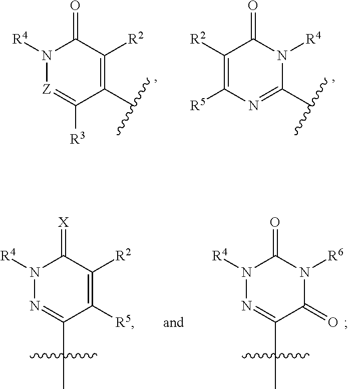

Ring B is an optionally substituted 5- to 6-membered heterocyclyl having 1-2 heteroatoms independently selected from N, O, and S;
L¹ is a covalent bond or an optionally substituted bivalent C₁₋₃ hydrocarbon chain;
each R¹ is independently —R;
n is 0, 1, 2, 3, or 4;
Z is N or CR³;
R² is halogen, —CN, or —R;
each R³ is independently halogen, —CN, or —R;
R⁴ is —R′;
R⁵ is halogen, —CN, or —R, or
   R² and R⁵, together with the atoms to which they are attached, combine to form an optionally substituted 5- to 6-membered ring having 0-2 heteroatoms independently selected from N, O, and S;
R⁶ is —R′;
X is O or NR⁷;
R⁷ is —R′, or
   R⁴ and R⁷, together with the atoms to which they are attached, combine to form an optionally substituted 5- to 6-membered ring having 2-3 heteroatoms independently selected from N, O, and S;
R⁹ is —R′;
linker is a covalent bond or an optionally substituted bivalent C₁-C₂₀ hydrocarbon chain, wherein one or more methylene units are optionally and independently replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —S(O)—, —SO₂—, —SO₂N(R)—, —N(R)SO₂—, or -Cy-;
each Cy is independently an optionally substituted, 3- to 16-membered bivalent ring having 0-6 heteroatoms independently selected from N, O, and S;
Ring F is an optionally substituted 3- to 16-membered ring having 0-6 heteroatoms independently selected from N, O, and S;
each R′ is independently —R, —C(O)R, or —S(O)₂R, or two R′ attached to the same atom, together with the atom to which they are attached, combine to form an optionally substituted 3- to 16-membered ring having 1-5 heteroatoms independently selected from N, O, and S;
each R is independently hydrogen or an optionally substituted group selected from C₁₋₈ aliphatic, C₃₋₁₀ cycloaliphatic, C₁-C₈ heteroaliphatic having 1-3 heteroatoms independently selected from N, O, and S, C₆₋₁₀ aryl, 5- to 10-membered heteroaryl having 1-5 heteroatoms independently selected from N, O, and S, and 3- to 10-membered monocyclic heterocyclyl having 1-5 heteroatoms independently selected from N, O, and S;
each R^{p} is independently halogen, —OR, —CN, or optionally substituted C₁₋₆ aliphatic;
each R^{q} is independently halogen, —OR, —CN, or optionally substituted C₁₋₆ aliphatic;
each R^{r} is independently hydrogen or optionally substituted C₁₋₆ aliphatic;
each R^{s} is independently halogen, —OR, —CN, or optionally substituted C₁₋₆ aliphatic;
t is 0, 1, 2, 3, 4, or 5; and
each u is independently 0, 1, 2, 3, 4, or 5.

In some embodiments, the present disclosure provides a compound of Formula VIIB:

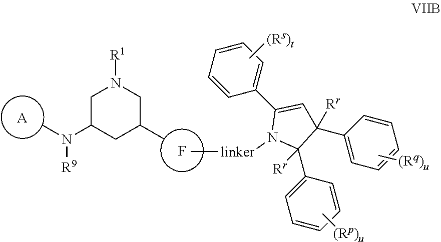

or a pharmaceutically acceptable salt thereof, wherein Ring A, R¹, R⁹, R^{p}, R^{q}, R^{r}, R^{s}, u, t, and linker are as defined above for Formula VII-1 and described in classes and subclasses herein, both singly and in combination.

In some embodiments, the present disclosure provides a compound of Formula VIII:

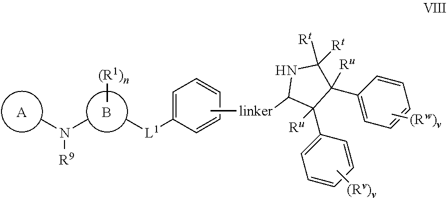

or a pharmaceutically acceptable salt thereof, wherein Ring A, Ring B, L¹, R¹, R⁹, n, and linker are as defined above for Formula II and described in classes and subclasses herein, both singly and in combination; and
each R^{t} is independently hydrogen or optionally substituted C₁₋₆ aliphatic, or
   both R^{t} groups, together with the atom to which they are attached, combine to form an optionally substituted 3- to 7-membered cycloaliphatic or heterocycle having 1-2 heteroatoms independently selected from N, O, and S;
each R^{u} is independently hydrogen, halogen, —CN, or optionally substituted C₁₋₆ aliphatic;
each R^{v} is independently halogen, —OR, —CN, or optionally substituted C₁₋₆ aliphatic, or
   one instance of R^{u} and R^{v}, together with the atoms to which they are attached combine to form an optionally substituted 3- to 7-membered cycloaliphatic or heterocycle having 1-2 heteroatoms independently selected from N, O, and S;
each R^{w} is independently halogen, —OR, —CN, or optionally substituted C₁₋₆ aliphatic, or
   one instance of R^{u} and R^{w}, together with the atoms to which they are attached combine to form an optionally substituted 3- to 7-membered cycloaliphatic or heterocycle having 1-2 heteroatoms independently selected from N, O, and S; and
each v is independently 0, 1, 2, 3, 4, or 5.

In some embodiments, the present disclosure provides a compound of Formula VIIIA:

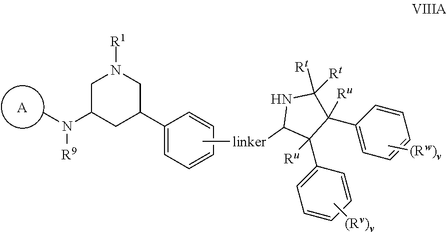

or a pharmaceutically acceptable salt thereof, wherein Ring A, R¹, R⁹, R^{t}, R^{u}, R^{v}, R^{w}, v, and linker are as defined above for Formula VIII and described in classes and subclasses herein, both singly and in combination.

In some embodiments, the present disclosure provides a compound of Formula VIII-1:

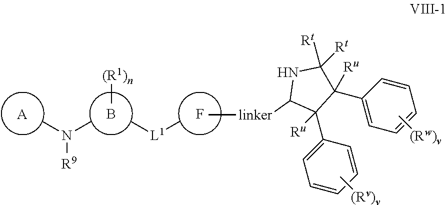

or a pharmaceutically acceptable salt thereof, wherein Ring A is selected from:

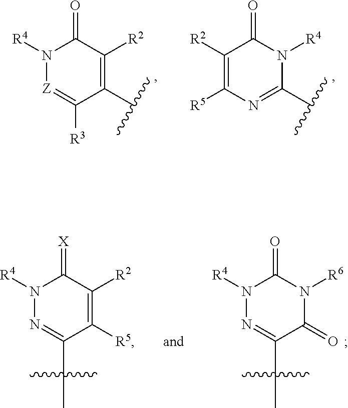

Ring B is an optionally substituted 5- to 6-membered heterocyclyl having 1-2 heteroatoms independently selected from N, O, and S;
L¹ is a covalent bond or an optionally substituted bivalent C₁₋₃ hydrocarbon chain;
each R¹ is independently —R;
n is 0, 1, 2, 3, or 4;
Z is N or CR³;
R² is halogen, —CN, or —R;
each R³ is independently halogen, —CN, or —R;
R⁴ is —R′;
R⁵ is halogen, —CN, or —R, or
   R² and R⁵, together with the atoms to which they are attached, combine to form an optionally substituted 5- to 6-membered ring having 0-2 heteroatoms independently selected from N, O, and S;
R⁶ is —R′;
X is O or NR⁷;
R⁷ is —R′, or
   R⁴ and R⁷, together with the atoms to which they are attached, combine to form an optionally substituted 5- to 6-membered ring having 2-3 heteroatoms independently selected from N, O, and S;
R⁹ is —R′;
linker is a covalent bond or an optionally substituted bivalent C₁-C₂₀ hydrocarbon chain, wherein one or more methylene units are optionally and independently replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —SC(O)N(R)—, —N(R)C(NR)N(R)—, —S(O)—, —SO₂—, —SO₂N(R)—, —N(R)SO₂—, or -Cy-;
   each Cy is independently an optionally substituted, 3- to 16-membered bivalent ring having 0-6 heteroatoms independently selected from N, O, and S;
   Ring F is an optionally substituted 3- to 16-membered ring having 0-6 heteroatoms independently selected from N, O, and S;
   each R′ is independently —R, —C(O)R, or —S(O)₂R, or two R′ attached to the same atom, together with the atom to which they are attached, combine to form an optionally substituted 3- to 16-membered ring having 1-5 heteroatoms independently selected from N, O, and S;
   each R is independently hydrogen or an optionally substituted group selected from C₁₋₈ aliphatic, C₃₋₁₀ cycloaliphatic, C₁-C₈ heteroaliphatic having 1-3 heteroatoms independently selected from N, O, and S, C₆₋₁₀ aryl, 5- to 10-membered heteroaryl having 1-5 heteroatoms independently selected from N, O, and S, and 3- to 10-membered monocyclic heterocyclyl having 1-5 heteroatoms independently selected from N, O, and S;
each R^{t} is independently hydrogen or optionally substituted C₁₋₆ aliphatic, or
   both R^{t} groups, together with the atom to which they are attached, combine to form an optionally substituted 3- to 7-membered cycloaliphatic or heterocycle having 1-2 heteroatoms independently selected from N, O, and S;
each R^{u} is independently hydrogen, halogen, —CN, or optionally substituted C₁₋₆ aliphatic;
each R^{v} is independently halogen, —OR, —CN, or optionally substituted C₁₋₆ aliphatic, or
   one instance of R^{u} and R^{v}, together with the atoms to which they are attached combine to form an optionally substituted 3- to 7-membered cycloaliphatic or heterocycle having 1-2 heteroatoms independently selected from N, O, and S;
each R^{w} is independently halogen, —OR, —CN, or optionally substituted C₁₋₆ aliphatic, or
   one instance of R^{u} and R^{w}, together with the atoms to which they are attached combine to form an optionally substituted 3- to 7-membered cycloaliphatic or heterocycle having 1-2 heteroatoms independently selected from N, O, and S; and
each v is independently 0, 1, 2, 3, 4, or 5.

In some embodiments, the present disclosure provides a compound of Formula VIIIB:

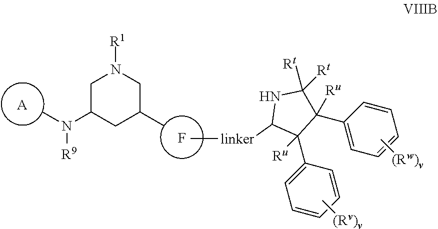

or a pharmaceutically acceptable salt thereof, wherein Ring A, Ring F, R¹, R⁹, R^{t}, R^{u}, R^{v}, R^{w}, v, and linker are as defined above for Formula VIII-1 and described in classes and subclasses herein, both singly and in combination.

In some embodiments, the present disclosure provides a compound of Formula IX:

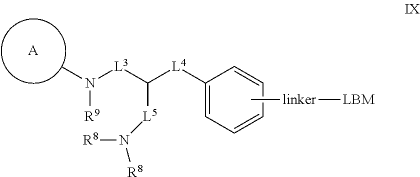

or a pharmaceutically acceptable salt thereof, wherein:
Ring A is selected from:

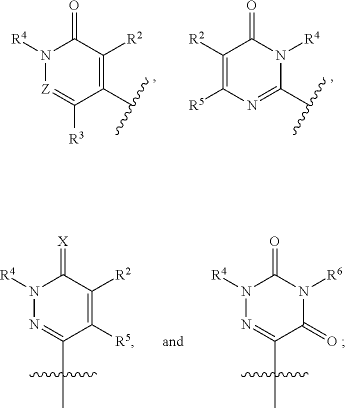

L³, L⁴, and L⁵ are each independently a covalent bond or an optionally substituted bivalent C₁₋₆ straight or branched hydrocarbon chain;
Z is N or CR³;
R² is hydrogen, halogen, —CN, optionally substituted C₁₋₆ aliphatic, or optionally substituted C₃₋₆ cycloaliphatic;
each R³ is independently hydrogen, halogen, optionally substituted C₁₋₆ aliphatic, or optionally substituted C₃₋₆ cycloaliphatic;
R⁴ is hydrogen, optionally substituted C₁₋₆ aliphatic, or optionally substituted C₃₋₆ cycloaliphatic;
R⁵ is hydrogen, halogen, optionally substituted C₁₋₆ aliphatic, or optionally substituted C₃₋₆ cycloaliphatic, or
   R² and R⁵, together with the atoms to which they are attached, combine to form an optionally substituted 5- to 6-membered aromatic ring having 0-2 heteroatoms independently selected from N, O, and S;
R⁶ is hydrogen, optionally substituted C₁₋₆ aliphatic, or optionally substituted C₃₋₆ cycloaliphatic;
X is O or NR⁷;
R⁷ is hydrogen or optionally substituted C₁₋₆ aliphatic, or
   R⁴ and R⁷, together with the atoms to which they are attached, combine to form an optionally substituted 5- to 6-membered aromatic ring having 2-3 heteroatoms independently selected from N, O, and S;
each R⁸ is independently hydrogen or optionally substituted C₁₋₆ aliphatic;
R⁹ is hydrogen or optionally substituted C₁₋₆ aliphatic;
linker is a covalent bond or an optionally substituted, bivalent, straight or branched, saturated or unsaturated C₁-C₂₀ hydrocarbon chain, wherein one or more methylene units are optionally and independently replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —SO₂, —SO₂N(R)—, —N(R)SO₂—, or -Cy-;
each Cy is independently an optionally substituted, mono- or multicyclic, 3- to 16-membered bivalent ring system, wherein the ring system is fully saturated, partially saturated, or aromatic, and the ring system contains 0-6 heteroatoms independently selected from N, O, and S;
each R is independently hydrogen or an optionally substituted group selected from C₁₋₆ aliphatic, phenyl, C₃₋₇ cycloaliphatic, 5- to 6-membered monocyclic heteroaryl having 1-4 heteroatoms independently selected from N, O, and S, and a 3- to 7-membered monocyclic heterocyclyl having 1-2 heteroatoms independently selected from N, O, and S; and
LBM is an E3 ubiquitin ligase binding moiety.

In some embodiments, the present disclosure provides a compound of Formula IX, or a pharmaceutically acceptable salt thereof, wherein:
Ring A is selected from:

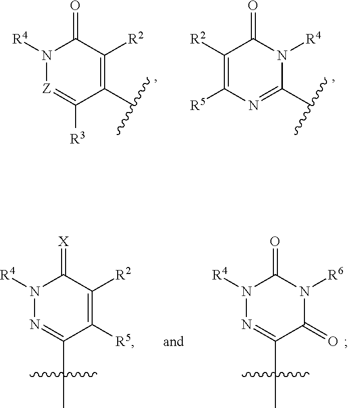

L³, L⁴, and L⁵ are each independently a covalent bond or an optionally substituted bivalent C₁₋₆ straight or branched hydrocarbon chain;
Z is N or CR³;
R³ is hydrogen, halogen, optionally substituted C₁₋₆ aliphatic, or optionally substituted C₃₋₆ cycloaliphatic;
each R³ is independently hydrogen, halogen, optionally substituted Che aliphatic, or optionally substituted C₃₋₆ cycloaliphatic;
R⁴ is hydrogen, optionally substituted C₁₋₆ aliphatic, or optionally substituted C₃₋₆ cycloaliphatic;
R⁵ is hydrogen, halogen, optionally substituted C₁₋₆ aliphatic, or optionally substituted C₃₋₆ cycloaliphatic, or
   R² and R⁵, together with the atoms to which they are attached, combine to form an optionally substituted 5- to 6-membered aromatic ring having 0-2 heteroatoms independently selected from N, O, and S;
R⁶ is hydrogen, optionally substituted C₁₋₆ aliphatic, or optionally substituted C₃₋₆ cycloaliphatic;
X is O or NR⁷;
R⁷ is hydrogen or optionally substituted C₁₋₆ aliphatic, or
   R⁴ and R⁷, together with the atoms to which they are attached, combine to form an optionally substituted 5- to 6-membered aromatic ring having 2-3 heteroatoms independently selected from N, O, and S;
each R⁸ is independently hydrogen or optionally substituted C₁₋₆ aliphatic;
R⁹ is hydrogen or optionally substituted C₁₋₆ aliphatic;
linker is a covalent bond or an optionally substituted, bivalent, straight or branched, saturated or unsaturated C₁-C₂₀ hydrocarbon chain, wherein one or more methylene units are optionally and independently replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —SO₂, —SO₂N(R)—, —N(R)SO₂—, or -Cy-;
each Cy is independently an optionally substituted, mono- or multicyclic, 3- to 16-membered bivalent ring system, wherein the ring system is fully saturated, partially saturated, or aromatic, and the ring system contains 0-6 heteroatoms independently selected from N, O, and S;
each R is independently hydrogen or an optionally substituted group selected from C₁₋₆ aliphatic, phenyl, C₃₋₇ cycloaliphatic, 5- to 6-membered monocyclic heteroaryl having 1-4 heteroatoms independently selected from N, O, and S, and a 3- to 7-membered monocyclic heterocyclyl having 1-2 heteroatoms independently selected from N, O, and S; and
LBM is an E3 ubiquitin ligase binding moiety.

In some embodiments, the present disclosure provides a compound of Formula IXA, IXB, IXC, or IXD:

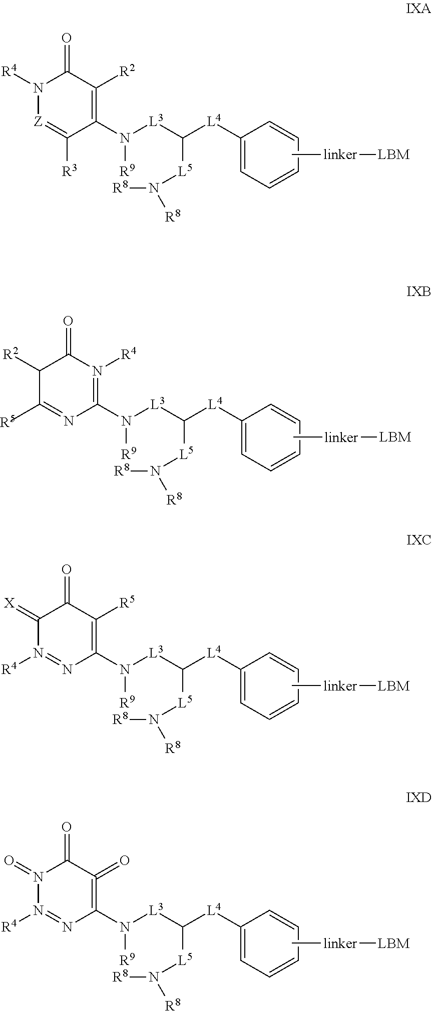

or a pharmaceutically acceptable salt thereof, wherein L³, L⁴, L⁵, R², R³, R⁴, R⁵, R⁶, R⁸, R⁹, X, Z, linker, and LBM are as defined above for Formula IX and described in classes and subclasses herein, both singly and in combination. In some embodiments, the present disclosure provides a compound of Formula IXA, or a pharmaceutically acceptable salt thereof. In some embodiments, the present disclosure provides a compound of Formula IXB, or a pharmaceutically acceptable salt thereof. In some embodiments, the present disclosure provides a compound of Formula IXC, or a pharmaceutically acceptable salt thereof. In some embodiments, the present disclosure provides a compound of Formula IXD, or a pharmaceutically acceptable salt thereof.

In some embodiments, the present disclosure provides a compound of Formula IX-1:

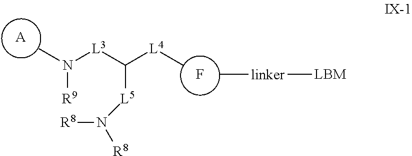

or a pharmaceutically acceptable salt thereof, wherein:
Ring A is selected from:

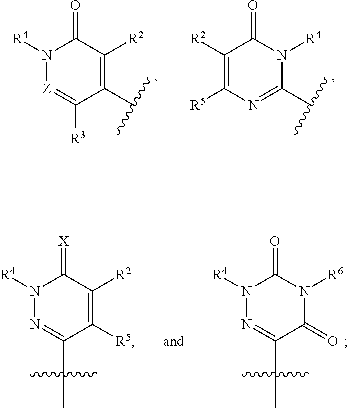

Z is N or CR³;
R² is halogen, —CN, or —R;
each R³ is independently halogen, —CN, or —R;
R⁴ is —R′;
R⁵ is halogen, —CN, or —R, or
   R² and R⁵, together with the atoms to which they are attached, combine to form an optionally substituted 5- to 6-membered ring having 0-2 heteroatoms independently selected from N, O, and S;
R⁶ is —R′;
X is O or NR⁷;
R⁷ is —R′, or
   R⁴ and R⁷, together with the atoms to which they are attached, combine to form an optionally substituted 5- to 6-membered ring having 2-3 heteroatoms independently selected from N, O, and S;
R⁹ is —R′;
L³, L⁴, and L⁵ are each independently a covalent bond or an optionally substituted bivalent C₁₋₆ hydrocarbon chain;
each R⁸ is independently —R′;
Ring F is an optionally substituted 3- to 16-membered ring having 0-6 heteroatoms independently selected from N, O, and S;
linker is a covalent bond or an optionally substituted, bivalent, straight or branched, saturated or linker is a covalent bond or an optionally substituted bivalent C₁-C₂₀ hydrocarbon chain, wherein one or more methylene units are optionally and independently replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —S(O)—, —SO₂—, —SO₂N(R)—, —N(R)SO₂—, or -Cy-;
each Cy is independently an optionally substituted, 3- to 16-membered bivalent ring having 0-6 heteroatoms independently selected from N, O, and S;
LBM is an E3 ubiquitin ligase binding moiety;
each R′ is independently —R, —C(O)R, or —S(O)₂R, or two R′ attached to the same atom, together with the atom to which they are attached, combine to form an optionally substituted 3- to 16-membered ring having 1-5 heteroatoms independently selected from N, O, and S; and
each R is independently hydrogen or an optionally substituted group selected from C₁₋₈ aliphatic, C₃₋₁₀ cycloaliphatic, C₁-C₈ heteroaliphatic having 1-3 heteroatoms independently selected from N, O, and S, C₆₋₁₀ aryl, 5- to 10-membered heteroaryl having 1-5 heteroatoms independently selected from N, O, and S, and 3- to 10-membered monocyclic heterocyclyl having 1-5 heteroatoms independently selected from N, O, and S.

In some embodiments, the present disclosure provides a compound of Formula X:

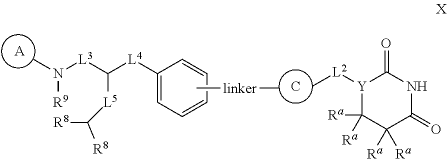

or a pharmaceutically acceptable salt thereof, wherein Ring A, L³, L⁴, L⁵, R⁸, R⁹, and linker are as defined above for Formula IX and described in classes and subclasses herein, both singly and in combination; and
Ring C is an optionally substituted, mono- or multicyclic, 3- to 16-membered bivalent ring system, wherein the ring system is fully saturated, partially saturated, or aromatic, and the ring system contains 0-6 heteroatoms independently selected from N, O, and S;
each R^{a} is independently hydrogen or an optionally substituted C₁₋₆ aliphatic, or two R^{a} groups, together with the atom(s) to which they are attached, combine to form a 3- to 6-membered saturated or partially unsaturated ring;
L² is a covalent bond or a straight or branched C₁₋₃ hydrocarbon chain wherein one methylene is optionally replaced with —O—, —S—, —N(R)—, —SO₂—, —C(O)N(R)—, or —N(R)C(O)—; and
Y is N or CH.

In some embodiments, the present disclosure provides a compound of Formula XA:

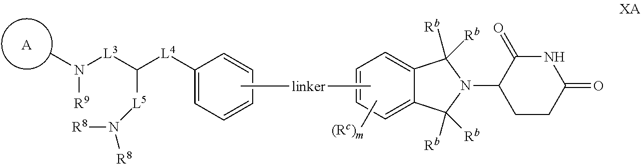

or a pharmaceutically acceptable salt thereof, wherein Ring A, L³, L⁴, L⁵, R⁸, R⁹, and linker are as defined above for Formula IX and described in classes and subclasses herein, both singly and in combination; and
each R^{b} is hydrogen, or two R^{b} groups, on the same carbon, are taken together to form an oxo or combine to form a 3- to 6-membered saturated or partially unsaturated ring;
each R^{c} is independently selected from halogen, ~OR, —N(R)₂, —CN, and optionally substituted C₁₋₆ aliphatic; and
m is 0, 1, 2, or 3.

In some embodiments, the present disclosure provides a compound of Formula XA-1 or XA-2:

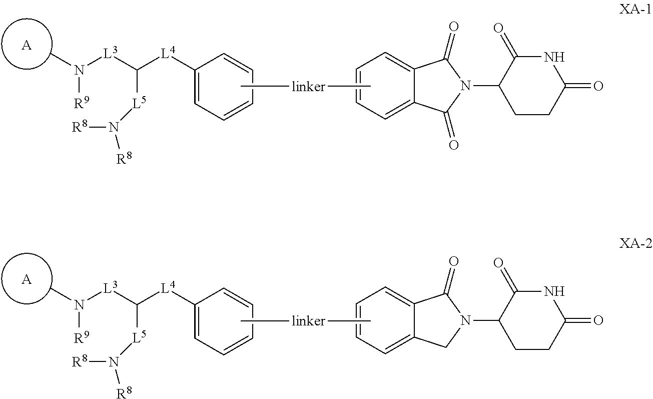

or a pharmaceutically acceptable salt thereof, wherein Ring A, L³, L⁴, L⁵, R⁸, R⁹, and linker are as defined above for Formula IX and described in classes and subclasses herein, both singly and in combination. In some embodiments, the present disclosure provides a compound of Formula XA-1, or a pharmaceutically acceptable salt thereof. In some embodiments, the present disclosure provides a compound of Formula XA-2, or a pharmaceutically acceptable salt thereof.

In some embodiments, the present disclosure provides a compound of Formula XB:

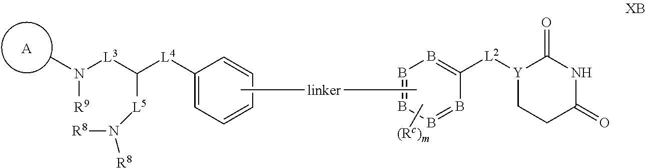

or a pharmaceutically acceptable salt thereof, wherein Ring A, L², L³, L⁴, L⁵, R⁸, R⁹, Y, and linker are as defined above for Formula X and described in classes and subclasses herein, both singly and in combination; and
each B is independently selected from N, C, and CH, provided that no more than two B are N;
each R^{c} is independently selected from halogen, —OR, —N(R)₂, —CN, and optionally substituted C₁₋₆ aliphatic; and
m is 0, 1, 2, or 3.

In some embodiments, the present disclosure provides a compound of Formula X-1, X-2, or X-3:

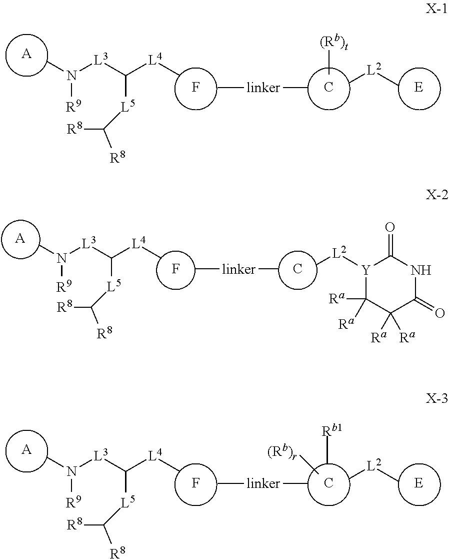

or a pharmaceutically acceptable salt thereof, wherein the variables are as defined as in formula I or IIIL and described in classes and subclasses herein, both singly and in combination.

In some embodiments, the present disclosure provides a compound of Formula XA′:

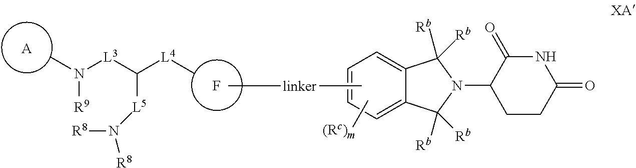

or a pharmaceutically acceptable salt thereof, wherein each R^{c} is independently selected from halogen, —OR, —N(R)₂, —CN, and —R; m is 0, 1, 2, or 3; and the other variables are as defined as in formula I and described in classes and subclasses herein, both singly and in combination. In some embodiments. Ring A is selected from:

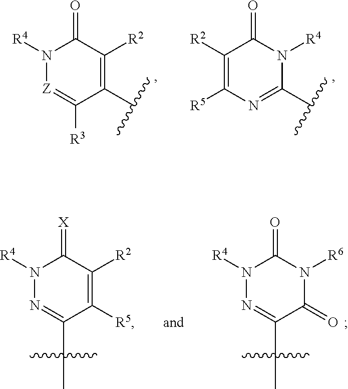

Z is N or CR³;
R² is halogen, —CN, or —R;
each R³ is independently halogen, —CN, or —R;
R⁴ is —R′;
R⁵ is halogen, —CN, or —R, or
   R² and R⁵, together with the atoms to which they are attached, combine to form an optionally substituted 5- to 6-membered ring having 0-2 heteroatoms independently selected from N, O, and S;
R⁶ is —R′;
X is O or NR⁷;
R⁷ is —R′, or
   R⁴ and R⁷, together with the atoms to which they are attached, combine to form an optionally substituted 5- to 6-membered ring having 2-3 heteroatoms independently selected from N, O, and S;
R⁹ is —R′;
L³, L⁴, and L⁵ are each independently a covalent bond or an optionally substituted bivalent C₁₋₆ hydrocarbon chain;
each R⁸ is independently —R′;
Ring F is an optionally substituted 3- to 16-membered ring having 0-6 heteroatoms independently selected from N, O, and S;
linker is a covalent bond or an optionally substituted, bivalent, straight or branched, saturated or linker is a covalent bond or an optionally substituted bivalent C₁-C₂₀ hydrocarbon chain, wherein one or more methylene units are optionally and independently replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —S(O)—, —SO₂—, —SO₂N(R)—, —N(R)SO₂—, or -Cy-;
each Cy is independently an optionally substituted, 3- to 16-membered bivalent ring having 0-6 heteroatoms independently selected from N, O, and S;
each R′ is independently —R, —C(O)R, or —S(O)₂R, or two R′ attached to the same atom, together with the atom to which they are attached, combine to form an optionally substituted 3- to 16-membered ring having 1-5 heteroatoms independently selected from N, O, and S;
each R is independently hydrogen or an optionally substituted group selected from C₁₋₈ aliphatic, C₃₋₁₀ cycloaliphatic, C₁-C₈ heteroaliphatic having 1-3 heteroatoms independently selected from N, O, and S, C₆₋₁₀ aryl, 5- to 10-membered heteroaryl having 1-5 heteroatoms independently selected from N, O, and S, and 3- to 10-membered monocyclic heterocyclyl having 1-5 heteroatoms independently selected from N, O, and S;
each R^{b} is hydrogen, or two R_{b} groups, on the same carbon, are taken together to form an oxo or combine to form a 3- to 6-membered saturated or partially unsaturated ring;
each R^{c} is independently selected from halogen, —OR, —N(R)₂, —CN, and optionally substituted C₁₋₆ aliphatic; and
m is 0, 1, 2, or 3.

In some embodiments, the present disclosure provides a compound of Formula XA-1′ or XA-2′:

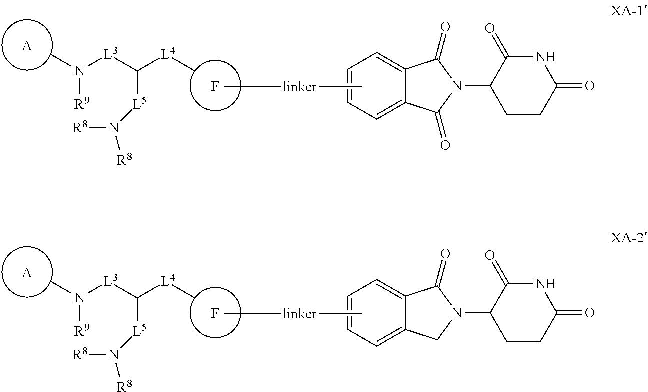

or a pharmaceutically acceptable salt thereof, wherein the variables are as defined as in formula I and described in classes and subclasses herein, both singly and in combination. In some embodiments, Ring A, L³, L⁴, L⁵, R⁸, R⁹, and linker are as defined above for Formula X-1 and described in classes and subclasses herein, both singly and in combination. In some embodiments, the present disclosure provides a compound of Formula XA-1′, or a pharmaceutically acceptable salt thereof. In some embodiments, the present disclosure provides a compound of Formula XA-2′, or a pharmaceutically acceptable salt thereof.

In some embodiments, the present disclosure provides a compound of Formula XB′:

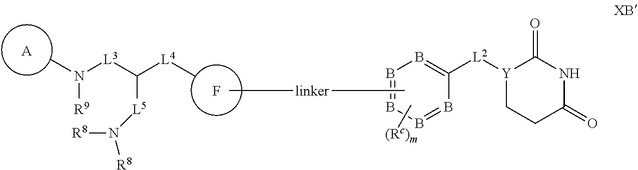

or a pharmaceutically acceptable salt thereof, wherein Ring A, Ring F, L², L³, L⁴, L⁵, R⁸, R⁹, Y, and linker are as defined above for Formula X-1 and described in classes and subclasses herein, both singly and in combination; and
each B is independently selected from N, C, and CH, provided that no more than two B are N;
each R^{c} is independently selected from halogen, —OR, —N(R)₂, —CN, and optionally substituted C₁₋₆ aliphatic; and
m is 0, 1, 2, or 3.

In some embodiments, the present disclosure provides a compound of Formula XI:

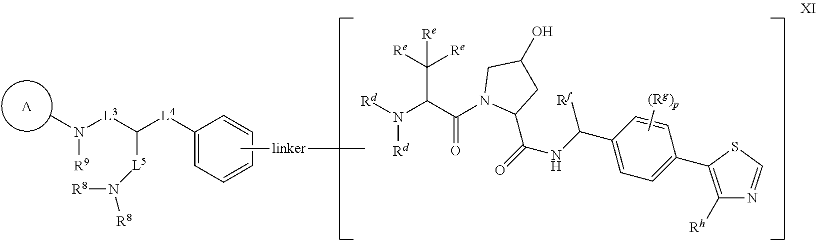

or a pharmaceutically acceptable salt thereof, wherein Ring A, L³, L⁴, L⁵, R⁸, R⁹, and linker are as defined above for Formula IX and described in classes and subclasses herein, both singly and in combination; and
linker is attached to the bracketed moiety at one of R^{d}, R^{e}, R^{f}, or R^{g};
each R^{d} that is not the point of attachment for the linker is independently hydrogen, —C(O)R, or optionally substituted C₁₋₆ aliphatic, or
   two R^{d}, together with the atom to which they are attached, combine to form an optionally substituted 5- to 6-membered ring having 1-3 heteroatoms independently selected from N, O, and S and optionally fused to a phenyl or 5- to 6-membered heteroaryl ring having 1-3 heteroatoms independently selected from N, O, and S;
each R^{e} that is not the point of attachment for the linker is independently hydrogen or optionally substituted C₁₋₆ aliphatic;
R^{f}, when it is not the point of attachment for the linker, is hydrogen or optionally substituted C₁₋₆ aliphatic;
each R^{g} that is not the point of attachment for the linker is independently halogen, —OR, —CN, or optionally substituted C₁₋₆ aliphatic;
R^{h} is hydrogen, halogen, or optionally substituted C₁₋₆ aliphatic; and
p is 0, 1, 2, or 3.

In some embodiments, the present disclosure provides a compound of Formula XIA:

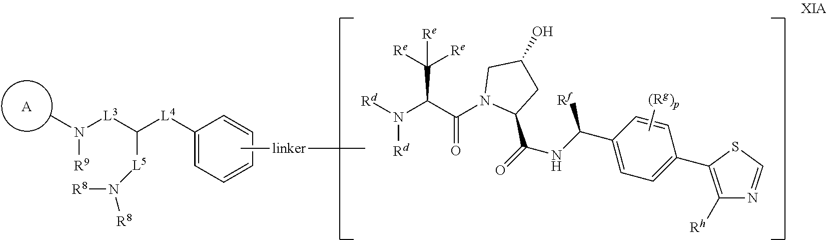

or a pharmaceutically acceptable salt thereof, wherein Ring A, L³, L⁴, L⁵, R⁸, R⁹, R^{d}, R^{e}, R^{f}, R^{g}, R^{h}, p, and linker are as defined above for Formula XI and described in classes and subclasses herein, both singly and in combination.

In some embodiments, the present disclosure provides a compound of Formula XI-1:

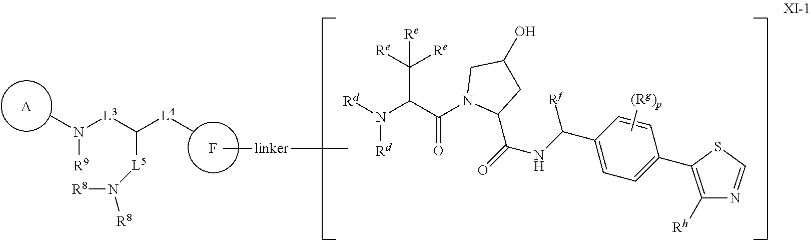

or a pharmaceutically acceptable salt thereof, wherein the variables are as defined as in formula I and described in classes and subclasses herein, both singly and in combination.

In some embodiments, the present disclosure provides a compound of Formula XIB:

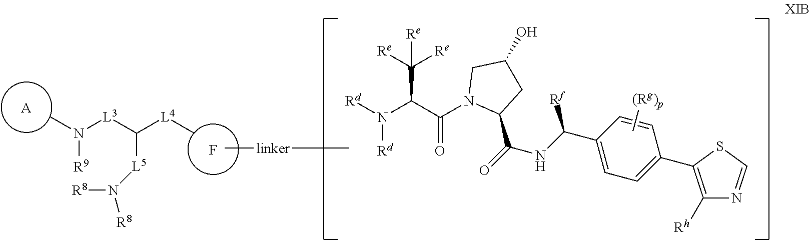

or a pharmaceutically acceptable salt thereof, wherein the variables are as defined as in formula I and described in classes and subclasses herein, both singly and in combination.

In some embodiments, the present disclosure provides a compound of Formula XII:

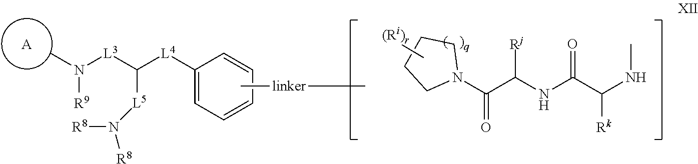

or a pharmaceutically acceptable salt thereof, wherein Ring A, L³, L⁴, L⁵, R⁸, R⁹, and linker are as defined above for Formula IX and described in classes and subclasses herein, both singly and in combination; and
linker is attached to the bracketed moiety at one Rⁱ or the ring formed when two Rⁱ groups are taken together;
each Rⁱ that is not the point of attachment of the linker is independently halogen, optionally substituted C₁₋₆ aliphatic, —C(O)N(R)₂, or —N(R)C(O)R, or
   two Rⁱ groups, together with the atoms to which they are attached, combine to form an optionally substituted phenyl or 5- to 6-membered heteroaryl ring having 1-3 heteroatoms independently selected from N, O, and S;
R^{j} is an optionally substituted group selected from C₁₋₆ aliphatic and C₃₋₇ cycloaliphatic, or
   R^{j} combines with one instance of Rⁱ, together with the atoms to which they are attached, to form an optionally substituted 5- to 7-membered heterocycle having 1-2 heteroatoms independently selected from N, O, and S;
R^{k} is an optionally substituted C₁₋₆ aliphatic;
r is 1, 2, 3, 4, or 5; and
q is 1 or 2.

In some embodiments, the present disclosure provides a compound of Formula XIIA:

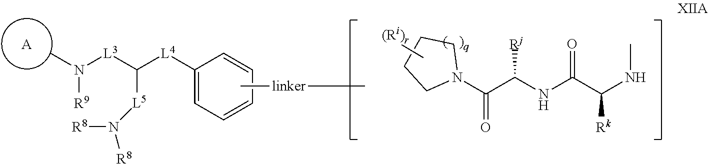

or a pharmaceutically acceptable salt thereof, wherein Ring A, L³, L⁴, L⁵, R⁸, R⁹, Rⁱ, R^{j}, R^{k}, q, r, and linker are as defined above for Formula XII and described in classes and subclasses herein, both singly and in combination.

In some embodiments, the present disclosure provides a compound of Formula XIIB:

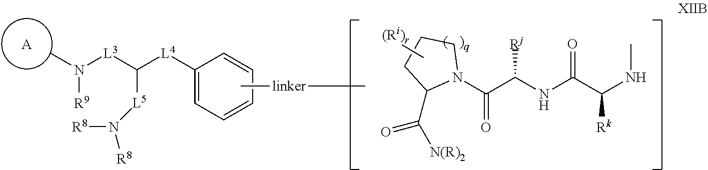

or a pharmaceutically acceptable salt thereof, wherein Ring A, L³, L⁴, L⁵, R, R⁸, R⁹, Rⁱ, R^{j}, R^{k}, q, r, and linker are as defined above for Formula XII and described in classes and subclasses herein, both singly and in combination.

In some embodiments, the present disclosure provides a compound of Formula XIIB-1:

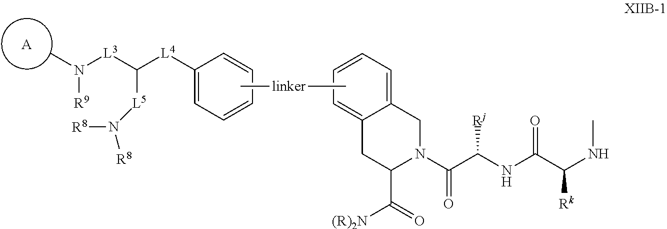

or a pharmaceutically acceptable salt thereof, wherein Ring A, L³, L⁴, L⁵, R, R⁸, R⁹, R^{j}, R^{k}, and linker are as defined above for Formula XII and described in classes and subclasses herein, both singly and in combination.

In some embodiments, the present disclosure provides a compound of Formula XII-1:

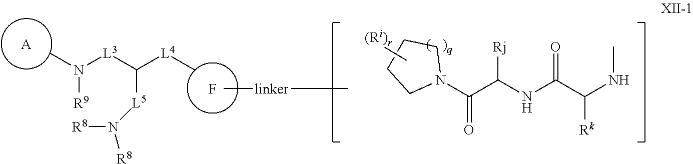

or a pharmaceutically acceptable salt thereof, wherein the variables are as defined as in formula I and described in classes and subclasses herein, both singly and in combination.

In some embodiments, the present disclosure provides a compound of Formula XIIA′:

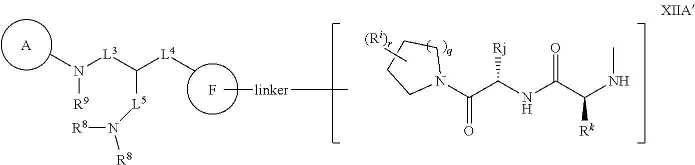

or a pharmaceutically acceptable salt thereof, wherein the variables are as defined as in formula I and described in classes and subclasses herein, both singly and in combination.

In some embodiments, the present disclosure provides a compound of Formula XIIB′:

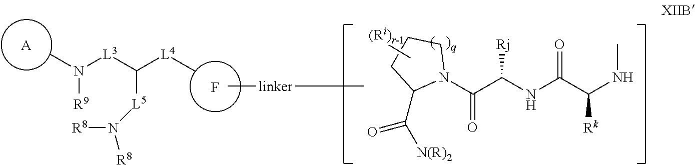

or a pharmaceutically acceptable salt thereof, wherein the variables are as defined as in formula I and described in classes and subclasses herein, both singly and in combination.

In some embodiments, the present disclosure provides a compound of Formula XIIB-1′:

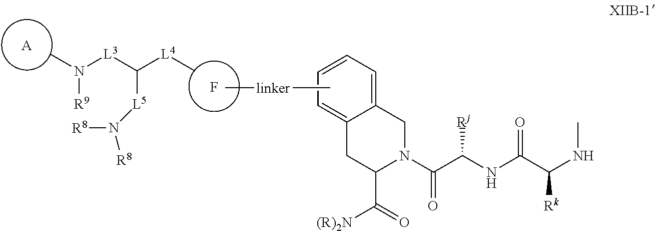

or a pharmaceutically acceptable salt thereof, wherein the variables are as defined as in formula I and described in classes and subclasses herein, both singly and in combination.

In some embodiments, the present disclosure provides a compound of Formula XIII:

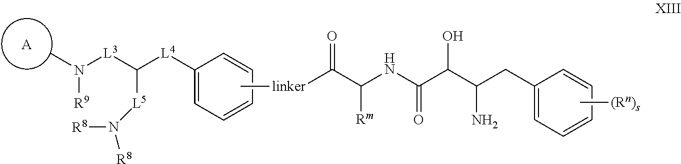

or a pharmaceutically acceptable salt thereof, wherein Ring A, L³, L⁴, L⁵, R⁸, R⁹, and linker are as defined above for Formula IX and described in classes and subclasses herein, both singly and in combination; and
R^{m} is optionally substituted C₁₋₆ aliphatic;
each Rⁿ is independently halogen, —OR, —CN, or optionally substituted C₁₋₆ aliphatic; and
s is 0, 1, 2, 3, 4, or 5.

In some embodiments, the present disclosure provides a compound of Formula XIIIA:

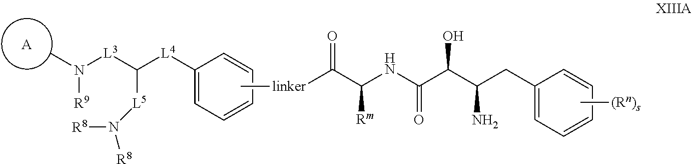

or a pharmaceutically acceptable salt thereof, wherein Ring A, L³, L⁴, L⁵, R⁸, R⁹, R^{m}, Rⁿ, s, and linker are as defined above for Formula XIII and described in classes and subclasses herein, both singly and in combination.

In some embodiments, the present disclosure provides a compound of Formula XIII-1:

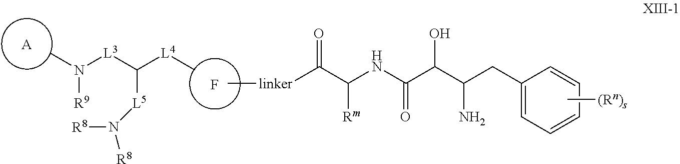

or a pharmaceutically acceptable salt thereof, wherein the variables are as defined as in formula I and described in classes and subclasses herein, both singly and in combination.

In some embodiments, the present disclosure provides a compound of Formula XIIIB:

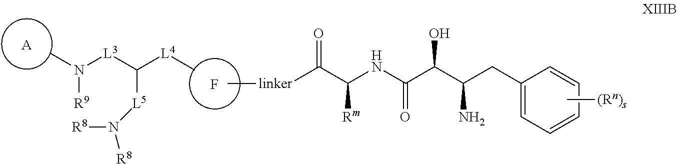

or a pharmaceutically acceptable salt thereof, wherein Ring A, Ring F, L³, L⁴, L⁵, R⁸, R⁹, R^{m}, Rⁿ, s, and linker are as defined above for Formula XIII-1 and described in classes and subclasses herein, both singly and in combination.

In some embodiments, the present disclosure provides a compound of Formula XIV:

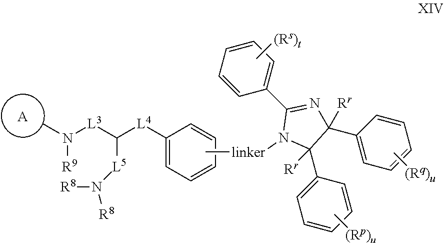

or a pharmaceutically acceptable salt thereof, wherein Ring A, L³, L⁴, L⁵, R⁸, R⁹, and linker are as defined above for Formula IX and described in classes and subclasses herein, both singly and in combination; and
each R^{p} is independently halogen, —OR, —CN, or optionally substituted C₁₋₆ aliphatic;
each R^{q} is independently halogen, —OR, —CN, or optionally substituted C₁₋₆ aliphatic;
each R^{r} is independently hydrogen or optionally substituted C₁₋₆ aliphatic;
each R^{s} is independently halogen, —OR, —CN, or optionally substituted C₁₋₆ aliphatic;
t is 0, 1, 2, 3, 4, or 5; and
each u is independently 0, 1, 2, 3, 4, or 5.

In some embodiments, the present disclosure provides a compound of Formula XIV-1:

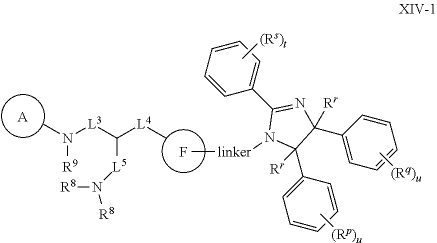

or a pharmaceutically acceptable salt thereof, wherein the variables are as defined as in formula I and described in classes and subclasses herein, both singly and in combination.

In some embodiments, the present disclosure provides a compound of Formula XV:

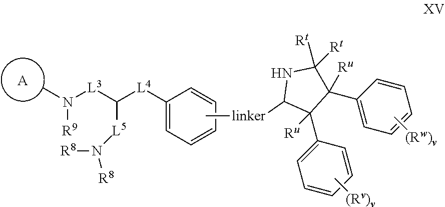

or a pharmaceutically acceptable salt thereof, wherein Ring A, L³, L⁴, L⁵, R⁸, R⁹, and linker are as defined above for Formula IX and described in classes and subclasses herein, both singly and in combination; and
each R^{t} is independently hydrogen or optionally substituted C₁₋₆ aliphatic, or
   both R^{t} groups, together with the atom to which they are attached, combine to form an optionally substituted 3- to 7-membered cycloaliphatic or heterocycle having 1-2 heteroatoms independently selected from N, O, and S;
each R^{u} is independently hydrogen, halogen, —CN, or optionally substituted C₁₋₆ aliphatic;
each R^{v} is independently halogen, —OR, —CN, or optionally substituted C₁₋₆ aliphatic, or
   one instance of R^{u} and R^{v}, together with the atoms to which they are attached combine to form an optionally substituted 3- to 7-membered cycloaliphatic or heterocycle having 1-2 heteroatoms independently selected from N, O, and S;
each R^{w} is independently halogen, —OR, —CN, or optionally substituted C₁₋₆ aliphatic, or
   one instance of R^{u} and R^{w}, together with the atoms to which they are attached combine to form an optionally substituted 3- to 7-membered cycloaliphatic or heterocycle having 1-2 heteroatoms independently selected from N, O, and S;
each v is independently 0, 1, 2, 3, 4, or 5.

In some embodiments, the present disclosure provides a compound of Formula XV-1:

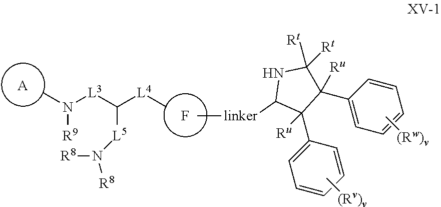

or a pharmaceutically acceptable salt thereof, wherein the variables are as defined as in formula I and described in classes and subclasses herein, both singly and in combination.

### PBM

In some embodiments of any Formula described herein, PBM is a KAT2 protein binding moiety. i.e., is a moiety that is capable of binding a KAT2 protein. In some embodiments, a PBM is a KAT2A protein binding moiety, i.e., is a moiety that is capable of binding a KAT2A protein. In some embodiments, a PBM is a KAT2B protein binding moiety, i.e., is a moiety that is capable of binding a KAT2B protein. Typically, a PBM is considered to be capable of binding a KAT2 protein (e.g., KAT2A and/or KAT2B) if it specifically (i.e., preferentially) associates with the KAT2 protein when contacted with the KAT2 protein in the presence of at least one other protein. In some embodiments, a PBM is considered to be capable of binding a KAT2 protein if it specifically associates with that protein within a cell (e.g., in vitro or in vivo).

In some embodiments, a PBM shares significant structural identity with a reference compound or moiety thereof that is capable of binding a KAT2 protein. For example, in some embodiments, a PBM comprises the same or similar structure as a reference compound, except that a PBM comprises a point of attachment to a linker. In some embodiments, a reference compound is characterized by a K_{d} of less than 1 μM in a biophysical assay, such as surface plasmon resonance (SPR) or isothermal titration calorimetry (ITC). In some embodiments, a reference compound is characterized by a IC₅₀ of less than 1 μM in a competition or functional assay, such as time-resolved fluorescence resonance energy transfer (TR-FRET). In some embodiments, a reference compound is characterized by an DC₅₀ of less than 30 nM in the Western Blot assay of Example B1. In some embodiments, a reference compound binds to a KAT2 protein but does not cause KAT2 degradation when assessed, e.g., as described in Example B1. In some embodiments, a reference compound has the structure of PBM-H or a salt thereof. In some embodiments, a reference compound is a compound described in WO 2016/036954, WO 2016/036873, WO 2016/112298, Chaikuad, A., et al., J. Med. Chem., 2016, 59, 1648-53, Humphreys, P. G., et al., J. Med. Chem., 2017, 60, 2, 695-709, Moustakim, M., et al., Angew. Chem. Int. Ed., 2017, 56, 827-31, the entire contents of each of which are hereby incorporated by reference. A reference compound can be useful for assessing the binding properties of a PBM of the present disclosure, e.g., by comparison of binding data of a PBM of the present disclosure with binding data of a reference compound. In some embodiments, a reference compound can be incorporated as a PBM (see, e.g., GSK4027 incorporated into various compounds as PBM). In some embodiments, a PBM is as described in WO 2024/003533, US20230391745, WO2017/197046, or Bassi, Z., et al., ACS Chem. Biol. 2018, 13, 2862-2867, the entirety of each of which is incorporated herein by reference. In some embodiments, technologies for identifying or assessing PBM are described in one or more of such references. In some embodiments, an PBM is considered to be capable of binding a KAT2A protein if a compound of PBM-H or a salt thereof, or PBM-linker-H or a salt thereof, has a K_{D} toward a KAT2A protein of about or less than about 1 uM in an assay such as surface plasmon resonance (SPR) or isothermal titration calorimetry (ITC). In some embodiments, a compound of PBM-H or a salt thereof, or PBM-linker-H or a salt thereof, has an IC₅₀ of about or less than about 1 uM in a competition or functional assay, such as time-resolved fluorescence resonance energy transfer (TR-FRET).

In some embodiments, a reference compound is GSK4027:

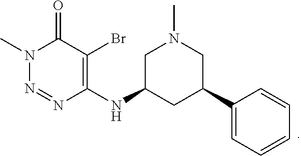 [SMILES: CN1C[C@H](Nc2nnn(C)c(=O)c2Br)C[C@H](c2ccccc2)C1]

In some embodiments, a reference compound is:

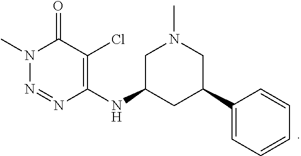 [SMILES: CN1C[C@H](Nc2nnn(C)c(=O)c2Cl)C[C@H](c2ccccc2)C1]

In some embodiments, a PBM has the structure of

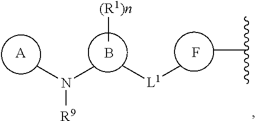

wherein the variables are as defined and described in classes and subclasses herein, both singly and in combination. In some embodiments, Ring F is an optionally substituted 3- to 10-membered ring having 0-4 heteroatoms independently selected from N, O, and S. In some embodiments, Ring F is an optionally substituted 5- to 10-(e.g., 5-8, 5-6, 3, 4, 5, 6, 7, 8, 9 or 10) membered ring having 0-4 heteroatoms independently selected from N, O, and S. In some embodiments, Ring F is an optionally substituted 5-membered ring having 0-4 heteroatoms independently selected from N, O, and S. In some embodiments, Ring F is an optionally substituted 6-membered ring having 0-4 heteroatoms independently selected from N, O, and S. In some embodiments, Ring F is an optionally substituted aromatic ring. In some embodiments, Ring F is an optionally substituted phenyl ring. In some embodiments, Ring F is an optionally substituted 5-membered heteroaryl ring having 1-4 heteroatoms independently selected from N, O and S. In some embodiments, Ring F is an optionally substituted 6-membered heteroaryl ring having 1-4 heteroatoms independently selected from N, O, and S. In some embodiments, Ring F is an optionally substituted 5- to 6-membered saturated or partially unsaturated ring having 0-2 heteroatoms independently selected from N, O, and S. In some embodiments, Ring F is an optionally substituted monocyclic ring (e.g., 3-10, 5-8, 3, 4, 5, 6, 7, 8, 9 or 10 membered). In some embodiments, Ring F is an optionally substituted bicyclic ring (e.g., 6-10, 6, 7, 8, 9 or 10 membered). In some embodiments, Ring F is polycyclic.

In some embodiments, PBM is

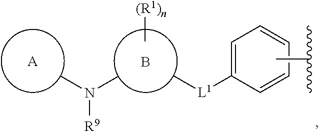

wherein each variable are as defined and described in classes and subclasses herein, both singly and in combination. In some embodiments, a PBM has the following structure:

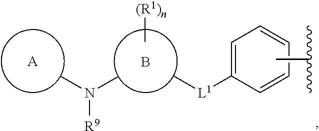

wherein Ring A, Ring B, L¹, R¹, R⁹, and n are as defined herein for Formula II and described in classes and subclasses herein, both singly and in combination.

In some embodiments, a PBM has the following structure:

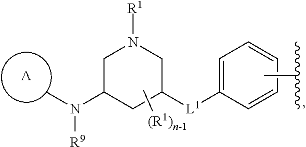

wherein Ring A, L¹, R¹, R⁹, and n are as defined herein for Formula II and described in classes and subclasses herein, both singly and in combination.

In some embodiments, a PBM has the following structure:

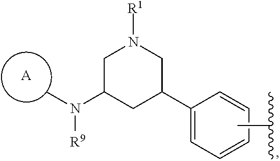

wherein Ring A, R¹, and R⁹ are as defined herein for Formula II and described in classes and subclasses herein, both singly and in combination.

In some embodiments, a PBM has the following structure:

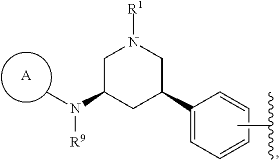

wherein Ring A, R¹, and R⁹ are as defined herein for Formula II and described in classes and subclasses herein, both singly and in combination.

In some embodiments, a PBM has the following structure:

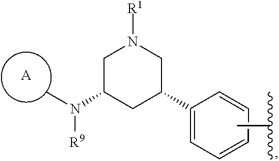

wherein Ring A, R¹, and R⁹ are as defined herein for Formula II and described in classes and subclasses herein, both singly and in combination.

In some embodiments, a PBM is selected from:

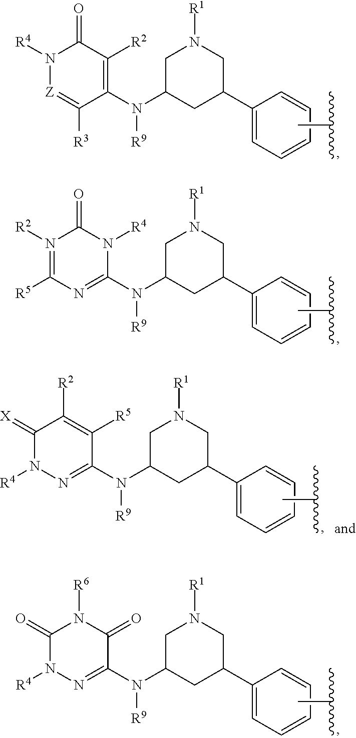

wherein R¹, R², R³, R⁴, R⁵, R⁶, R⁹, X, and Z are as defined herein for Formula II and described in classes and subclasses herein, both singly and in combination.

In some embodiments, a PBM has the following structure:

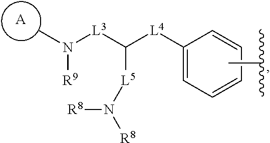

wherein Ring A, L³, L⁴, L⁵, R⁸, and R⁹ are as defined herein for Formula IX and described in classes and subclasses herein, both singly and in combination.

In some embodiments, a PBM is selected from:

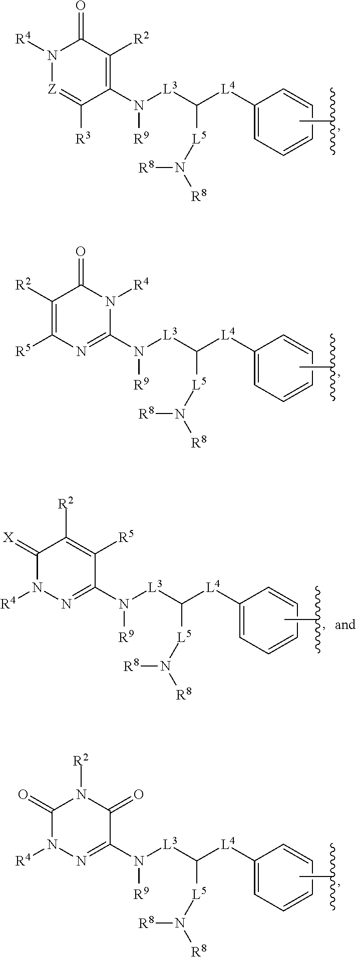

wherein L³, L⁴, L⁵, R², R³, R⁴, R⁵, R⁶, R⁸, R⁹, X, and Z are as defined herein for Formula IX and described in classes and subclasses herein, both singly and in combination.

In some embodiments, a PBM is selected from:

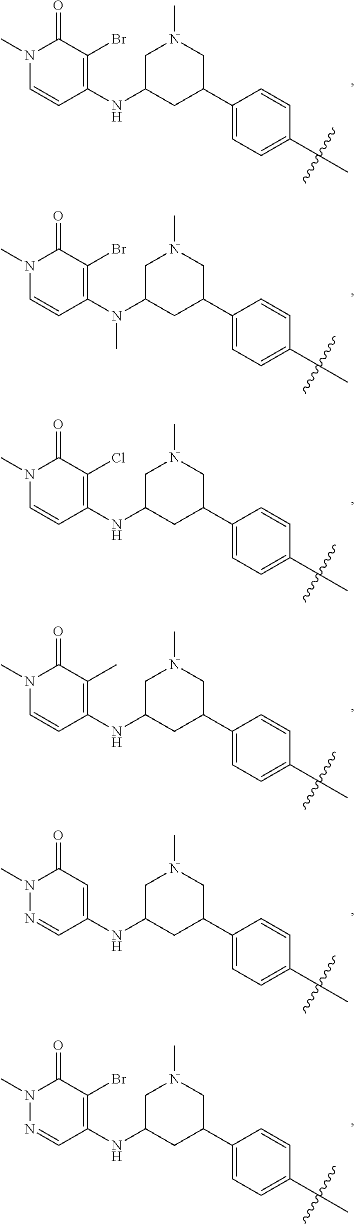 [SMILES: *c1ccc(C2CC(Nc3ccn(C)c(=O)c3Br)CN(C)C2)cc1 ; *c1ccc(C2CC(N(C)c3ccn(C)c(=O)c3Br)CN(C)C2)cc1 ; *c1ccc(C2CC(Nc3ccn(C)c(=O)c3Cl)CN(C)C2)cc1 ; *c1ccc(C2CC(Nc3ccn(C)c(=O)c3C)CN(C)C2)cc1 ; *c1ccc(C2CC(Nc3cnn(C)c(=O)c3)CN(C)C2)cc1 ; *c1ccc(C2CC(Nc3cnn(C)c(=O)c3Br)CN(C)C2)cc1]

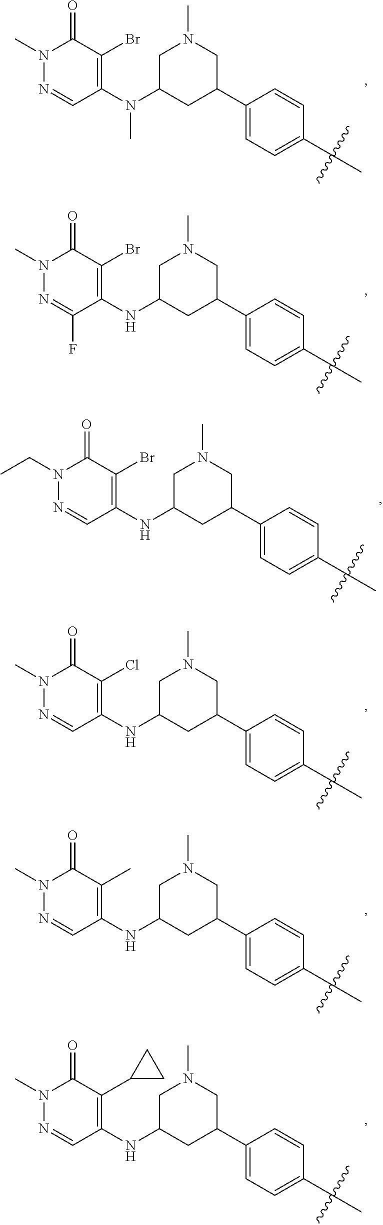 [SMILES: *c1ccc(C2CC(N(C)c3cnn(C)c(=O)c3Br)CN(C)C2)cc1 ; *c1ccc(C2CC(Nc3c(F)nn(C)c(=O)c3Br)CN(C)C2)cc1 ; *c1ccc(C2CC(Nc3cnn(CC)c(=O)c3Br)CN(C)C2)cc1 ; *c1ccc(C2CC(Nc3cnn(C)c(=O)c3Cl)CN(C)C2)cc1 ; *c1ccc(C2CC(Nc3cnn(C)c(=O)c3C)CN(C)C2)cc1 ; *c1ccc(C2CC(Nc3cnn(C)c(=O)c3C3CC3)CN(C)C2)cc1]

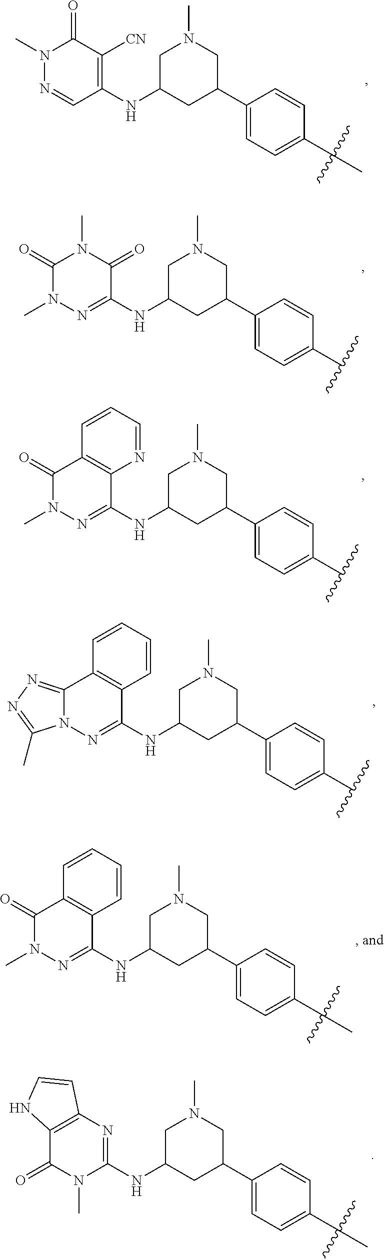 [SMILES: *c1ccc(C2CC(Nc3cnn(C)c(=O)c3C#N)CN(C)C2)cc1 ; *c1ccc(C2CC(Nc3nn(C)c(=O)n(C)c3=O)CN(C)C2)cc1 ; *c1ccc(C2CC(Nc3nn(C)c(=O)c4cccnc34)CN(C)C2)cc1 ; *c1ccc(C2CC(Nc3nn4c(C)nnc4c4ccccc34)CN(C)C2)cc1 ; *c1ccc(C2CC(Nc3nn(C)c(=O)c4ccccc34)CN(C)C2)cc1 ; *c1ccc(C2CC(Nc3nc4cc[nH]c4c(=O)n3C)CN(C)C2)cc1]

In some embodiments, a PBM is selected from:

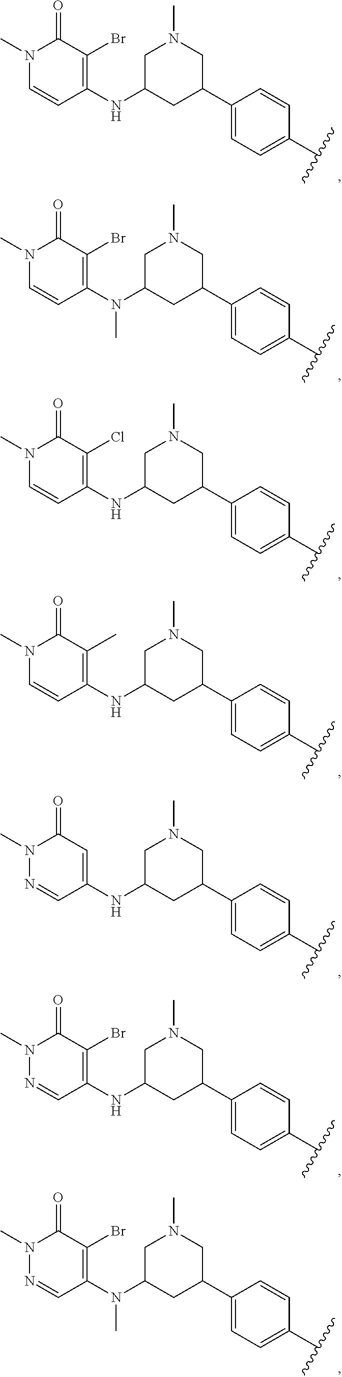

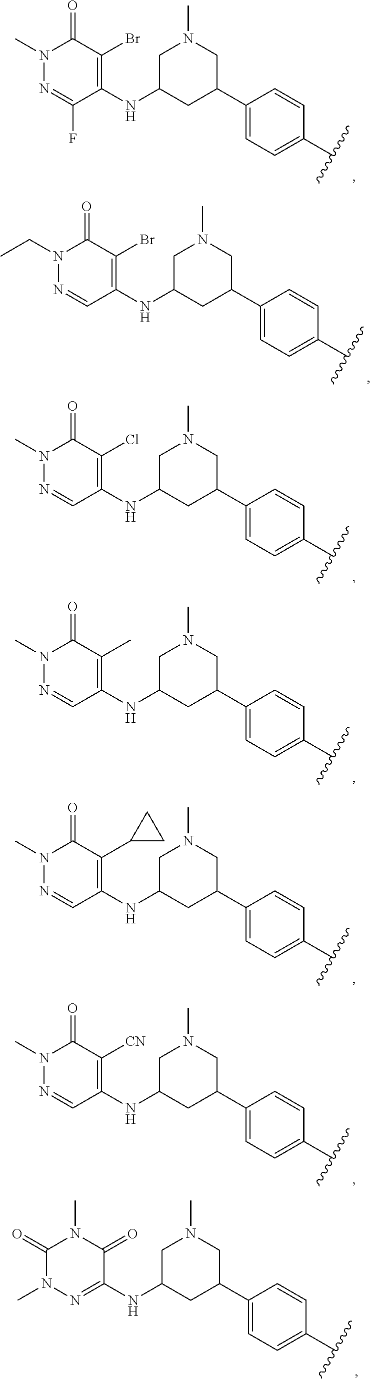

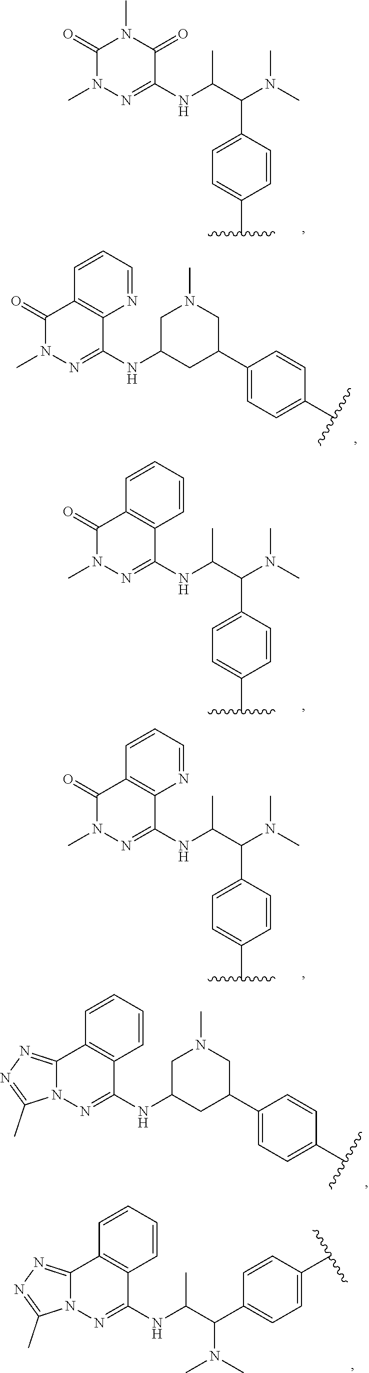 [SMILES: *c1ccc(C(C(C)Nc2nn(C)c(=O)n(C)c2=O)N(C)C)cc1 ; *c1ccc(C2CC(Nc3nn(C)c(=O)c4cccnc34)CN(C)C2)cc1 ; *c1ccc(C(C(C)Nc2nn(C)c(=O)c3ccccc23)N(C)C)cc1 ; *c1ccc(C(C(C)Nc2nn(C)c(=O)c3cccnc23)N(C)C)cc1 ; *c1ccc(C2CC(Nc3nn4c(C)nnc4c4ccccc34)CN(C)C2)cc1 ; *c1ccc(C(C(C)Nc2nn3c(C)nnc3c3ccccc23)N(C)C)cc1]

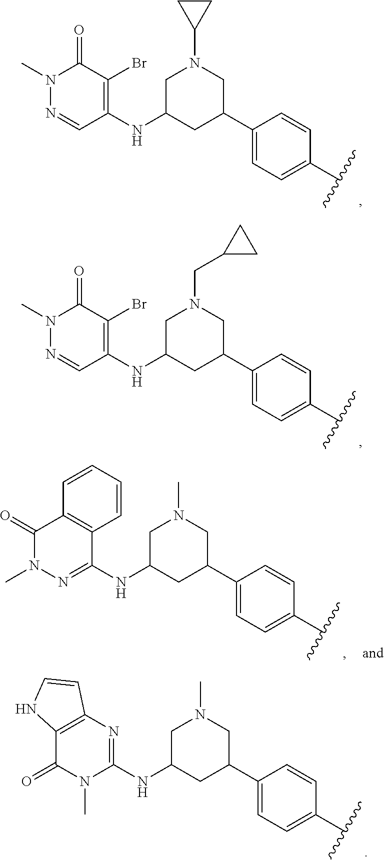 [SMILES: *c1ccc(C2CC(Nc3cnn(C)c(=O)c3Br)CN(C3CC3)C2)cc1 ; *c1ccc(C2CC(Nc3cnn(C)c(=O)c3Br)CN(CC3CC3)C2)cc1 ; *c1ccc(C2CC(Nc3nn(C)c(=O)c4ccccc34)CN(C)C2)cc1 ; *c1ccc(C2CC(Nc3nc4cc[nH]c4c(=O)n3C)CN(C)C2)cc1]

In some embodiments of any Formula described herein (unless explicitly indicated otherwise, with or without “of any Formula described herein” or similar language, embodiments described, e.g., for variables or moieties, should apply to all suitable formulae, structures, etc., e.g., all of those comprising such variables or moieties), Ring A is

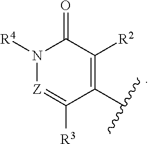

In some embodiments, Ring A is

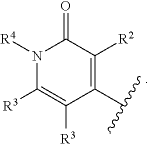

In some embodiments, Ring A is

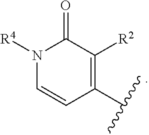

In some embodiments, Ring A is

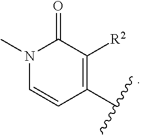

In some embodiments, Ring A is

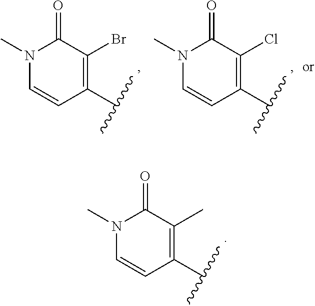

In some embodiments, Ring A is

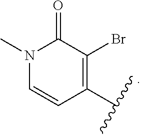 [SMILES: *c1ccn(C)c(=O)c1Br]

In some embodiments, Ring A is

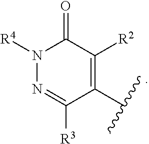

In some embodiments, Ring A is

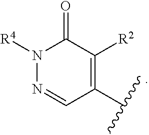

In some embodiments, Ring A is

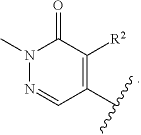

In some embodiments, Ring A is selected from:

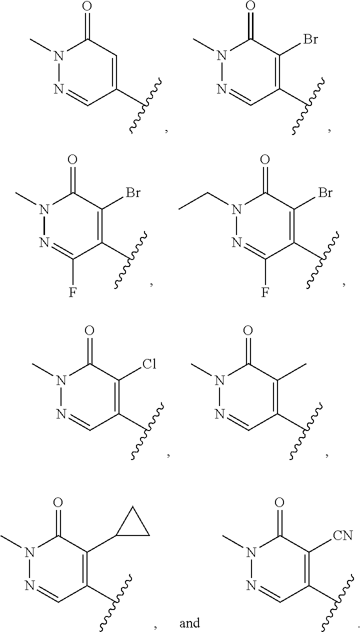

In some embodiments, Ring A is selected from:

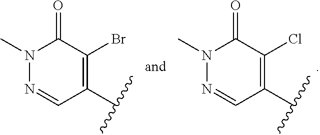

In some embodiments of any Formula described herein, Ring A is

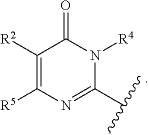

In some embodiments, Ring A is

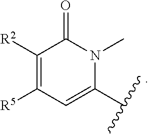

In some such embodiments, R² and R⁵, together with the atoms to which they are attached, combine to form an optionally substituted 5- to 6-membered ring having 0-2 heteroatoms independently selected from N, O, and S. In some embodiments, a formed ring is aromatic. In some embodiments, Ring A is

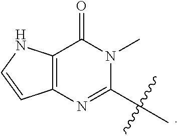 [SMILES: *c1nc2cc[nH]c2c(=O)n1C]

In some embodiments of any Formula described herein, Ring A is

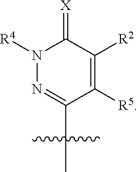

In some embodiments, Ring A is

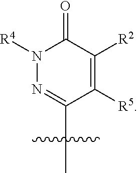

In some embodiments, Ring A is

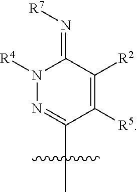

In some such embodiments, R² and R⁵, together with the atoms to which they are attached, combine to form an optionally substituted 5- to 6-membered aromatic ring having 0-2 heteroatoms independently selected from N, O, and S. In some such embodiments, R⁴ and R⁷, together with the atoms to which they are attached, combine to form an optionally substituted 5- to 6-membered ring having 2-3 heteroatoms independently selected from N, O, and S. In some embodiments, a formed ring is aromatic. In some embodiments, Ring A is selected from:

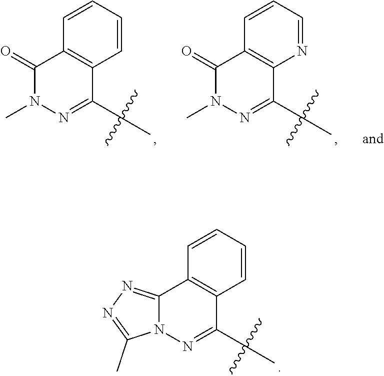 [SMILES: *c1nn(C)c(=O)c2ccccc12 ; *c1nn(C)c(=O)c2cccnc12 ; *c1nn2c(C)nnc2c2ccccc12]

In some embodiments of any Formula described herein, Ring A is

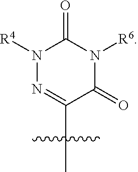

In some embodiments, Ring A is

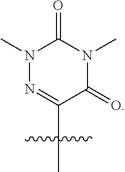

In some embodiments of any Formula described herein, Z is N or CH. In some embodiments, Z is N. In some embodiments, Z is CR³ (e.g., CH). In some embodiments, Z is optionally substituted CH.

In some embodiments, Ring F is an optionally substituted 3- to 16-membered (e.g., 3, 4, 5, 6, 7, 8, 9, 10, 11, 12, 13, 14, 15, 16, 3-15, 3-10, 5-10, 3-8, 3-7, 5-6 membered) ring having 0-6 (e.g., 0, 1-6, 1-4, 1, 2, 3, 4, 5, or 6) heteroatoms independently selected from N, O, and S. In some embodiments, Ring F is 3-10 membered. In some embodiments, it is 3-membered. In some embodiments, it is 4-membered. In some embodiments, it is 5-membered. In some embodiments, it is 6-membered. In some embodiments, it is 7-membered. In some embodiments, it is 8-membered. In some embodiments, it is 9-membered. In some embodiments, it is 10-membered. In some embodiments, it is saturated. In some embodiments, it is partially unsaturated. In some embodiments, it is aromatic. In some embodiments, it has 1-6, 1-5, 1-4, 1-3, 1, 2, 3, or 4 heteroatoms. In some embodiments, it has no heteroatom. In some embodiments, it is monocyclic. In some embodiments, it is bicyclic. In some embodiments, it is polycyclic. In some embodiments, at least one monocyclic ring unit is saturated. In some embodiments, at least one monocyclic ring unit is partially unsaturated. In some embodiments, at least one monocyclic ring unit is aromatic. In some embodiments, at least one monocyclic ring unit has a heteroatom. In some embodiments, at least one monocyclic ring unit has no heteroatom. In some embodiments, Ring F is an optionally substituted 5- to 6-membered ring having 0-3 heteroatoms independently selected from N, O, and S. In some embodiments, Ring F is an optionally substituted phenyl ring. In some embodiments, Ring F is an optionally substituted pyridine ring. In some embodiments, Ring F is an optionally substituted 5-membered ring having 1-3 heteroatoms independently selected from N, O, and S. In some embodiments, Ring F is an optionally substituted 5-membered heteroaryl ring having 1-3 heteroatoms independently selected from N, O, and S. In some embodiments, Ring F is an optionally substituted 6-membered ring having 1-3 heteroatoms independently selected from N, O, and S. In some embodiments, Ring F is an optionally substituted 6-membered heteroaryl ring having 1-3 heteroatoms independently selected from N, O, and S. In some embodiments, Ring F is 3-10 membered. In some embodiments, it is 3-membered. In some embodiments, it is 4-membered. In some embodiments, it is 5-membered. In some embodiments, it is 6-membered. In some embodiments, it is 7-membered. In some embodiments, it is 8-membered. In some embodiments, it is 9-membered. In some embodiments, it is 10-membered. In some embodiments, it is saturated. In some embodiments, it is partially unsaturated. In some embodiments, it is aromatic. In some embodiments, it has 1-6, 1-5, 1-4, 1-3, 1, 2, 3, or 4 heteroatoms. In some embodiments, it has no heteroatom. In some embodiments, it is monocyclic. In some embodiments, it is bicyclic. In some embodiments, it is polycyclic. In some embodiments, at least one monocyclic ring unit is saturated. In some embodiments, at least one monocyclic ring unit is partially unsaturated. In some embodiments, at least one monocyclic ring unit is aromatic. In some embodiments, at least one monocyclic ring unit has a heteroatom. In some embodiments, at least one monocyclic ring unit has no heteroatom.

In some embodiments, Ring F is optionally substituted with one or more halogens. In some embodiments, Ring F is optionally substituted with one or more C₁-C₆ alkyl groups. In some embodiments, Ring F is optionally substituted with an optionally substituted —O(C₁-C₆ alkyl) group. In some embodiments, Ring F is

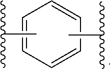

In some embodiments, Ring F is optionally substituted 1,4-phenylene. In some embodiments, Ring F is 1,4-phenylene. In some embodiments, Ring F is

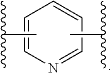

In some embodiments, Ring F is

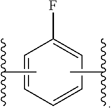

In some embodiments, R² is —R as described herein. In some embodiments, R² is —R but not hydrogen. In some embodiments of any Formula described herein, R² is hydrogen, halogen, —CN, or optionally substituted C₁₋₆ aliphatic. In some embodiments, R² is hydrogen, halogen, —CN, C₁₋₆ alkyl, or C₃₋₆ cycloalkyl. In some embodiments, R² is hydrogen, halogen, —CN, C₁₋₆ alkyl, C₃₋₆ cycloalkyl, or C₆₋₁₀ aryl. In some embodiments, R² is hydrogen, halogen, —CN, C₁₋₆ alkyl, C₃₋₆ cycloalkyl, C₆₋₁₀ aryl, or 5- to 10-membered heteroaryl having 1-5 heteroatoms independently selected from N, O, and S. In some embodiments, R² is hydrogen, halogen, —CN, C₁₋₆ alkyl, C₃₋₆ cycloalkyl, C₆₋₁₀ aryl, 5- to 10-membered heteroaryl having 1-5 heteroatoms independently selected from N, O, and S, or 3- to 10-membered monocyclic heterocyclyl having 1-5 heteroatoms independently selected from N, O, and S. In some embodiments, R² is hydrogen, halogen, or optionally substituted C₁₋₆ aliphatic. In some embodiments, R² is halogen or C₁₋₆ alkyl. In some embodiments, R² is chloro, bromo, or methyl. In some embodiments, R² is hydrogen. In some embodiments, R² is halogen. In some embodiments, R² is fluoro. In some embodiments, R² is chloro. In some embodiments, R² is bromo. In some embodiments, R² is-CN. In some embodiments, R² is optionally substituted Cho aliphatic. In some embodiments, R² is optionally substituted C₁₋₆ alkyl. In some embodiments, R² is C₁₋₂ alkyl (e.g., methyl or ethyl). In some embodiments, R² is optionally substituted C₃₋₆ cycloaliphatic. In some embodiments, R² is optionally substituted C₃₋₆ cycloalkyl. In some embodiments. R² is C₃₋₄ cycloalkyl (e.g., cyclopropyl). In some embodiments, R² is C₆₋₁₀ aryl. In some embodiments, R² is phenyl. In some embodiments, R² is naphthyl. In some embodiments, R² is 5- to 10-membered heteroaryl having 1-5 heteroatoms independently selected from N, O, and S. In some embodiments. R² is 5- to 6-membered heteroaryl having 1-3 heteroatoms independently selected from N, O, and S. In some embodiments, R² is 3- to 10-membered monocyclic heterocyclyl having 1-5 heteroatoms independently selected from N, O, and S. In some embodiments, R² is 4- to 6-membered monocyclic heterocyclyl having 1-3 heteroatoms independently selected from N, O, and S.

In some embodiments, R³ is —R as described herein. In some embodiments, R³ is —R but not H. In some embodiments of any Formula described herein, each R³ is independently hydrogen, halogen, or optionally substituted C₁₋₆ aliphatic. In some embodiments, each R³ is independently hydrogen, halogen, —CN, C₁₋₆ alkyl, or C₃₋₆ cycloalkyl. In some embodiments, each R³ is independently hydrogen, halogen, —CN, C₁₋₆ alkyl, C₃₋₆ cycloalkyl, or C₆₋₁₀ aryl. In some embodiments, each R³ is independently hydrogen, halogen, —CN, C₁₋₆ alkyl, C₃₋₆ cycloalkyl, C₆₋₁₀ aryl, or 5- to 10-membered heteroaryl having 1-5 heteroatoms independently selected from N, O, and S. In some embodiments, each R³ is independently hydrogen, halogen, —CN, C₁₋₆ alkyl, C₃₋₆ cycloalkyl, C₆₋₁₀ aryl, 5- to 10-membered heteroaryl having 1-5 heteroatoms independently selected from N, O, and S, or 3- to 10-membered monocyclic heterocyclyl having 1-5 heteroatoms independently selected from N, O, and S. In some embodiments, each R³ is hydrogen. In some embodiments, R³ is hydrogen. In some embodiments. R³ is halogen. In some embodiments, R³ is fluoro. In some embodiments, R³ is chloro. In some embodiments, R³ is bromo. In some embodiments, R³ is optionally substituted C₁₋₆ aliphatic. In some embodiments, R³ is optionally substituted C₁₋₆ alkyl. In some embodiments, R³ is C₁₋₂ alkyl (e.g., methyl). In some embodiments, R³ is optionally substituted C₃₋₆ cycloaliphatic. In some embodiments, R³ is optionally substituted C₃₋₆ cycloalkyl. In some embodiments, R³ is C₆₋₁₀ aryl. In some embodiments, R³ is phenyl. In some embodiments. R³ is naphthyl. In some embodiments, R³ is 5- to 10-membered heteroaryl having 1-5 heteroatoms independently selected from N, O, and S. In some embodiments, R³ is 5- to 6-membered heteroaryl having 1-3 heteroatoms independently selected from N, O, and S. In some embodiments. R³ is 3- to 10-membered monocyclic heterocyclyl having 1-5 heteroatoms independently selected from N, O, and S. In some embodiments, R³ is 4- to 6-membered monocyclic heterocyclyl having 1-3 heteroatoms independently selected from N, O, and S.

In some embodiments, R⁴ is —R as described herein. In some embodiments, R⁴ is —R but not hydrogen. In some embodiments of any Formula described herein, R⁴ is hydrogen or optionally substituted C₁₋₆ aliphatic. In some embodiments, R⁴ is hydrogen. In some embodiments, R⁴ is optionally substituted C₁₋₆ aliphatic. In some embodiments, R⁴ is hydrogen. In some embodiments, R⁴ is optionally substituted C₁₋₆ aliphatic. In some embodiments. R⁴ is optionally substituted C₁₋₆ alkyl. In some embodiments, R⁴ is C₁₋₆ alkyl. In some embodiments, R⁴ is optionally substituted C₁₋₂ alkyl. In some embodiments, R⁴ is C₁₋₂ alkyl (e.g., methyl). In some embodiments, R³ is methyl. In some embodiments, R⁴ is optionally substituted C₃₋₆ cycloaliphatic. In some embodiments, R⁴ is optionally substituted C₃₋₆ cycloalkyl. In some embodiments, R⁴ is C₃₋₄ cycloalkyl (e.g., cyclopropyl). In some embodiments. R⁴ is —C(O)R. In some embodiments, R⁴ is —C(O)(C₁₋₆ alkyl). In some embodiments, R⁴ is —S(O)₂R. In some embodiments, R⁴ is —S(O)₂(C₁₋₆ alkyl).

In some embodiments, R⁵ is —R as described herein. In some embodiments, R⁵ is —R but is not hydrogen. In some embodiments of any Formula described herein, R⁵ is hydrogen, halogen, or optionally substituted C₁₋₆ aliphatic. In some embodiments, R⁵ is hydrogen, halogen, —CN, C₁₋₆ alkyl, or C₃₋₆ cycloalkyl. In some embodiments, R⁵ is hydrogen, halogen, —CN, C₁₋₆ alkyl, C₃₋₆ cycloalkyl, or C₆₋₁₀ aryl. In some embodiments, R³ is hydrogen, halogen, —CN, C₁₋₆ alkyl, C₃₋₆ cycloalkyl, C₆₋₁₀ aryl, or 5- to 10-membered heteroaryl having 1-5 heteroatoms independently selected from N, O, and S. In some embodiments, R⁵ is hydrogen, halogen, —CN, C₁₋₆ alkyl, C₃₋₆ cycloalkyl, C₆₋₁₀ aryl, 5- to 10-membered heteroaryl having 1-5 heteroatoms independently selected from N, O, and S, or 3- to 10-membered monocyclic heterocyclyl having 1-5 heteroatoms independently selected from N, O, and S. In some embodiments. R⁵ is hydrogen. In some embodiments, R⁵ is halogen. In some embodiments, R⁵ is fluoro. In some embodiments, R⁵ is chloro. In some embodiments, R⁵ is bromo. In some embodiments, R⁵ is optionally substituted C₁₋₆ aliphatic. In some embodiments, R⁵ is optionally substituted C₁₋₆ alkyl. In some embodiments, R⁵ is C₁₋₂ alkyl (e.g., methyl). In some embodiments, R⁵ is optionally substituted C₃₋₆ cycloaliphatic. In some embodiments, R⁵ is optionally substituted C₃₋₆ cycloalkyl. In some embodiments, R⁵ is C₃₋₄ cycloalkyl (e.g., cyclopropyl). In some embodiments, R⁵ is C₆₋₁₀ aryl. In some embodiments, R⁵ is phenyl. In some embodiments, R⁵ is naphthyl. In some embodiments. R⁵ is 5- to 10-membered heteroaryl having 1-5 heteroatoms independently selected from N, O, and S. In some embodiments, R⁵ is 5- to 6-membered heteroaryl having 1-3 heteroatoms independently selected from N, O, and S. In some embodiments, R⁵ is 3- to 10-membered monocyclic heterocyclyl having 1-5 heteroatoms independently selected from N, O, and S. In some embodiments, R⁵ is 4- to 6-membered monocyclic heterocyclyl having 1-3 heteroatoms independently selected from N, O, and S.

In some embodiments, R² and R⁵, together with the atoms to which they are attached, combine to form an optionally substituted 5- to 6-membered ring having 0-2 heteroatoms independently selected from N, O, and S. In some embodiments, a formed ring is monocyclic. In some embodiments, it is 5-membered. In some embodiments, it is 6-membered. In some embodiments, it has 1-2 heteroatoms. In some embodiments, it has no heteroatoms. In some embodiments, it is saturated. In some embodiments, it is partially unsaturated. In some embodiments, it is aromatic. In some embodiments, it is an optionally substituted heteroaryl ring having 1-2 heteroatoms independently selected from N, O, and S. In some embodiments of any Formula described herein, R² and R⁵, together with the atoms to which they are attached, combine to form an optionally substituted 5- to 6-membered aromatic ring having 0-2 heteroatoms independently selected from N, O, and S. In some embodiments, R² and R⁵, together with the atoms to which they are attached, combine to form an 5- to 6-membered aromatic ring having 0-2 heteroatoms independently selected from N, O, and S, and optionally substituted on a substitutable carbon atom with one or more halogen, —R^{∘}, —OR^{∘}, —N(R^{∘})₂, and —CN, and on a substitutable nitrogen atom with one or more —R^{†} and —C(O)R^{†}. In some embodiments. R² and R⁵, together with the atoms to which they are attached, combine to form an optionally substituted 5-membered aromatic ring having 1-2 heteroatoms independently selected from N, O, and S. In some embodiments, R² and R⁵ combine to form an optionally substituted pyrrole. In some embodiments, R² and R⁵, together with the atoms to which they are attached, combine to form an optionally substituted 6-membered aromatic ring having 0-2 heteroatoms independently selected from N, O, and S. In some embodiments, R² and R⁵ combine to form an optionally substituted phenyl or pyridine. In some embodiments, R² and R⁵ combine to form an optionally substituted phenyl ring. In some embodiments, R² and R⁵ combine to form an optionally substituted pyridine ring. In some embodiments, R² and R⁵ combine to form optionally substituted

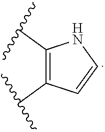 [SMILES: *c1cc[nH]c1*]

In some embodiments, R⁶ is —R as described herein. In some embodiments, R⁶ is —R but is not hydrogen. In some embodiments of any Formula described herein, R⁶ is hydrogen or optionally substituted C₁₋₆ aliphatic. In some embodiments, R⁶ is hydrogen. In some embodiments, R⁶ is optionally substituted C₁₋₆ aliphatic. In some embodiments, R⁶ is optionally substituted C₁₋₆ alkyl. In some embodiments, R⁶ is C₁₋₆ alkyl. In some embodiments, R⁶ is optionally substituted C₁₋₂ alkyl. In some embodiments, R⁶ is C₁₋₂ alkyl (e.g., methyl). In some embodiments, R⁶ is methyl. In some embodiments. R⁶ is optionally substituted C₃₋₆ cycloaliphatic. In some embodiments, R⁶ is optionally substituted C₃₋₆ cycloalkyl. In some embodiments. R⁶ is C₃₋₄ cycloalkyl (e.g., cyclopropyl). In some embodiments, R⁶ is —C(O)R. In some embodiments, R⁶ is —C(O)(C₁₋₆ alkyl). In some embodiments, R⁶ is —S(O)₂R. In some embodiments, R⁶ is —S(O)₂(C₁₋₆ alkyl).

In some embodiments of any Formula described herein, X is O. In some embodiments, X is NR⁷. In some embodiments, when X is NR⁷, then R⁴ and R⁷, together with the atoms to which they are attached, combine to form an optionally substituted 5- to 6-membered aromatic ring having 2-3 heteroatoms independently selected from N, O, and S.

In some embodiments, R⁷ is —R as described herein. In some embodiments, R′ is —R but is not hydrogen. In some embodiments of any Formula described herein, R⁷ is hydrogen or optionally substituted C₁₋₆ alkyl. In some embodiments, R⁷ is hydrogen. In some embodiments, R⁷ is optionally substituted C₁₋₆ aliphatic. In some embodiments, R⁷ is optionally substituted C₁₋₆ alkyl. In some embodiments, R⁷ is C₁₋₆ alkyl. In some embodiments, R⁷ is optionally substituted C₁₋₂ alkyl. In some embodiments, R⁷ is C₁₋₂ alkyl (e.g., methyl). In some embodiments, R⁷ is optionally substituted C₃₋₆ cycloaliphatic. In some embodiments, R⁷ is optionally substituted C₃₋₆ cycloalkyl. In some embodiments, R⁷ is C₃₋₄ cycloalkyl (e.g., cyclopropyl). In some embodiments, R⁷ is —C(O)R. In some embodiments, R⁷ is —C(O)(C₁₋₆ alkyl). In some embodiments, R⁷ is —S(O)₂R. In some embodiments, R⁷ is —S(O)₂(C₁₋₆ alkyl).

In some embodiments, R⁴ and R⁷, together with the atoms to which they are attached, combine to form an optionally substituted 5- to 6-membered ring having 2-3 heteroatoms independently selected from N, O, and S. In some embodiments, a formed ring is monocyclic. In some embodiments, it is 5-membered. In some embodiments, it is 6-membered. In some embodiments, it has 2 heteroatoms. In some embodiments, it has 3 heteroatoms. In some embodiments, it is saturated. In some embodiments, it is partially unsaturated. In some embodiments, it is aromatic. In some embodiments of any Formula described herein, R⁴ and R⁷, together with the atoms to which they are attached, combine to form an optionally substituted 5- to 6-membered aromatic ring having 2-3 heteroatoms independently selected from N, O, and S. In some embodiments, R⁴ and R⁷, together with the atoms to which they are attached, combine to form a 5- to 6-membered aromatic ring having 2-3 heteroatoms independently selected from N, O, and S, and optionally substituted on a substitutable carbon atom with one or more halogen, —R^{∘}, —OR^{∘}, —N(R^{∘})₂, and —CN, and on a substitutable nitrogen atom with one or more —R^{†} and —C(O)R^{†}. In some embodiments, R⁴ and R⁷, together with the atoms to which they are attached, combine to form a 5- to 6-membered aromatic ring having 2-3 heteroatoms independently selected from N, O, and S, and optionally substituted with one or more C₁₋₆ alkyl. In some embodiments, R⁴ and R⁷, together with the atoms to which they are attached, combine to form a 5-membered aromatic ring having 2-3 heteroatoms independently selected from N, O, and S. In some embodiments, R⁴ and R⁷ combine to form an optionally substituted triazole. In some embodiments, R⁴ and R⁷, together with the atoms to which they are attached, combine to form a 6-membered aromatic ring having 2-3 heteroatoms independently selected from N, O, and S. In some embodiments, a formed ring is optionally substituted

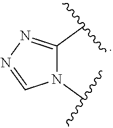 [SMILES: *c1nncn1*]

In some embodiments, a formed ring is

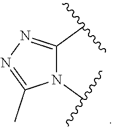 [SMILES: *c1nnc(C)n1*]

In some embodiments, R⁹ is —R as described herein. In some embodiments, R⁹ is —R but is not hydrogen. In some embodiments of any Formula described herein, R⁹ is hydrogen or optionally substituted C₁₋₆ alkyl. In some embodiments, R⁹ is hydrogen. In some embodiments, R⁹ is optionally substituted C₁₋₆ aliphatic. In some embodiments, R⁹ is optionally substituted C₁₋₆ alkyl. In some embodiments, R⁹ is C₁₋₆ alkyl. In some embodiments, R⁹ is optionally substituted C₁₋₂ alkyl. In some embodiments, R⁹ is C₁₋₂ alkyl (e.g., methyl). In some embodiments, R⁹ is methyl. In some embodiments, R⁹ is optionally substituted C₃₋₆ cycloaliphatic. In some embodiments, R⁹ is optionally substituted C₃₋₆ cycloalkyl. In some embodiments, R⁹ is C₃₋₄ cycloalkyl (e.g., cyclopropyl). In some embodiments, R⁹ is —C(O)R. In some embodiments, R⁹ is —C(O)(C₁₋₆ alkyl). In some embodiments, R⁹ is —S(O)₂R. In some embodiments, R⁹ is —S(O)₂(C₁₋₆ alkyl).

In some embodiments, Ring B is an optionally substituted 5-membered heterocyclyl having 1-2 heteroatoms independently selected from N, O, and S. In some embodiments, Ring B is an optionally substituted 5-membered saturated heterocyclyl having 1-2 heteroatoms independently selected from N, O, and S. In some embodiments, Ring B is an optionally substituted 5-membered partially unsaturated heterocyclyl having 1-2 heteroatoms independently selected from N, O, and S. In some embodiments, Ring B is an optionally substituted 6-membered heterocyclyl having 1-2 heteroatoms independently selected from N, O, and S. In some embodiments, Ring B is an optionally substituted 9-membered saturated heterocyclyl having 1-2 heteroatoms independently selected from N, O, and S. In some embodiments, Ring B is an optionally substituted 5-membered partially unsaturated heterocyclyl having 1-2 heteroatoms independently selected from N, O, and S. In some embodiments, Ring B has at least one nitrogen atom. In some embodiments, Ring B is an optionally substituted piperidine ring. In some embodiments, Ring B is optionally substituted

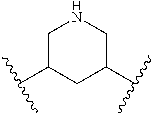 [SMILES: *C1CNCC(*)C1]

In some embodiments, Ring B is

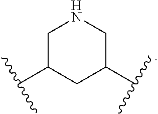 [SMILES: *C1CNCC(*)C1]

In some embodiments of any Formula described herein, Ring B is a 5- to 6-membered heterocyclyl having 1 heteroatom independently selected from N, O, and S. In some embodiments, Ring B is a 5- to 6-membered heterocyclyl having 1-2 heteroatoms independently selected from N, O, and S, wherein at least one heteroatom is N. In some embodiments, Ring B is a 5-membered heterocyclyl having 1-2 heteroatoms independently selected from N, O, and S. In some embodiments, Ring B is a 6-membered heterocyclyl having 1-2 heteroatoms independently selected from N, O, and S. In some embodiments, Ring B is piperidine. In some embodiments,

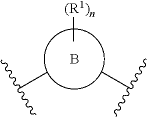

is

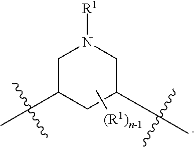

In some embodiments,

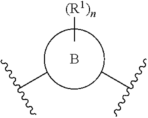

is

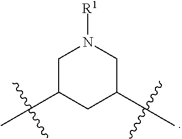

In some embodiments of any Formulae described herein, L¹ is a covalent bond. In some embodiments, L¹ is an optionally substituted bivalent C₁₋₃ hydrocarbon chain. In some embodiments, L¹ is an optionally substituted bivalent saturated C₁₋₃ hydrocarbon chain. In some embodiments, L¹ is an optionally substituted bivalent partially unsaturated C₁₋₃ hydrocarbon chain. In some embodiments, L¹ is an optionally substituted bivalent linear C₁₋₃ hydrocarbon chain. In some embodiments, L¹ is an optionally substituted bivalent branched C₁₋₃ hydrocarbon chain. In some embodiments, L¹ is a bivalent C₁₋₃ straight or branched hydrocarbon chain. In some embodiments, L¹ is a bivalent Cia straight hydrocarbon chain. In some embodiments, L¹ is a bivalent Cis straight hydrocarbon chain. In some embodiments, L¹ is optionally substituted In some embodiments, L¹ is —CH₂—.

In some embodiments, R¹ is —R as described herein. In some embodiments, R¹ is —R but not hydrogen. In some embodiments of any Formulae described herein, each R¹ is independently optionally substituted C₁₋₆ alkyl or optionally substituted C₃₋₆ cycloalkyl. In some embodiments, each R¹ is independently hydrogen or an optionally substituted group selected from C₁₋₈ aliphatic, C₅₋₁₀ aryl, C₃₋₁₀ cycloaliphatic, 5- to 10-membered heteroaryl having 1-5 heteroatoms independently selected from N, O, and S, and a 3- to 10-membered monocyclic heterocyclyl having 1-5 heteroatoms independently selected from N, O, and S. In some embodiments, R¹ is optionally substituted C₁₋₆ aliphatic. In some embodiments, R¹ is optionally substituted C₁₋₆ alkyl. In some embodiments, R¹ is C₁₋₆ alkyl. In some embodiments, R¹ is optionally substituted C₁₋₂ alkyl. In some embodiments, R¹ is C₁₋₂ alkyl (e.g., methyl). In some embodiments, R¹ is methyl. In some embodiments, R¹ is optionally substituted C₃₋₆ cycloaliphatic. In some embodiments, R¹ is optionally substituted C₃₋₆ cycloalkyl. In some embodiments, R¹ is C₃₋₄ cycloalkyl (e.g., cyclopropyl). In some embodiments, R¹ is —CH₂—C₃₋₆ cycloaliphatic. In some embodiments, R¹ is —CH₂-cyclopropyl. In some embodiments, R¹ is C₆₋₁₀ aryl. In some embodiments, R¹ is phenyl. In some embodiments, R¹ is naphthyl. In some embodiments, R¹ is 5- to 10-membered heteroaryl having 1-5 heteroatoms independently selected from N, O, and S. In some embodiments, R¹ is 5- to 6-membered heteroaryl having 1-3 heteroatoms independently selected from N, O, and S. In some embodiments, R¹ is 3- to 10-membered monocyclic heterocyclyl having 1-5 heteroatoms independently selected from N, O, and S. In some embodiments, R¹ is 4- to 6-membered monocyclic heterocyclyl having 1-3 heteroatoms independently selected from N, O, and S.

In some embodiments of any Formulae described herein, n is 0, 1, 2, 3, or 4. In some embodiments, n is 0 or 1. In some embodiments, n is 1, 2, or 3. In some embodiments, n is 0. In some embodiments, n is 1. In some embodiments, n is 2. In some embodiments, n is 3. In some embodiments, n is 4.

In some embodiments, L³ is an optionally substituted bivalent straight C₁₋₆ (e.g., C₁₋₃, C₁, C₂, C₃, C₄, C₅ or C₆) hydrocarbon chain. In some embodiments, L³ is an optionally substituted bivalent branched C₁₋₆ (e.g., C₁₋₃, C₁, C₂, C₃, C₄, C₅ or C₆) hydrocarbon chain. In some embodiments of any Formulae described herein, L³ is a covalent bond or an optionally substituted bivalent C₁₋₃ straight or branched hydrocarbon chain. In some embodiments, L³ is a covalent bond. In some embodiments, L³ is a bivalent Cie straight or branched hydrocarbon chain. In some embodiments, L³ is an optionally substituted bivalent C₁₋₃ straight or branched hydrocarbon chain. In some embodiments, L³ is a bivalent C₁₋₃ straight or branched hydrocarbon chain. In some embodiments, L³ is optionally substituted —CH₂—. In some embodiments, L³ is selected from —CH₂— and —CH(CH₃)—. In some embodiments, a chain is saturated. In some embodiments, it is partially unsaturated. In some embodiments, L³ is —CH₂—.

In some embodiments, L⁴ is an optionally substituted bivalent straight C₁₋₆ (e.g., C₁₋₃, C₁, C₂, C₃, C₄, C₅ or C₆) hydrocarbon chain. In some embodiments, L⁴ is an optionally substituted bivalent branched C₁₋₆ (e.g., C₁₋₃, C₁, C₂, C₃, C₄, C₅ or C₆) hydrocarbon chain. In some embodiments of any Formulae described herein, L⁴ is a covalent bond or an optionally substituted bivalent C₁₋₃ straight or branched hydrocarbon chain. In some embodiments, L⁴ is a covalent bond. In some embodiments, L⁴ is an optionally substituted bivalent C₁₋₆ straight or branched hydrocarbon chain. In some embodiments, L⁴ is a bivalent C₁₋₆ straight or branched hydrocarbon chain. In some embodiments, L⁴ is an optionally substituted bivalent C₁₋₃ straight or branched hydrocarbon chain. In some embodiments, L⁴ is a bivalent C₁₋₃ straight or branched hydrocarbon chain. In some embodiments, L⁴ is optionally substituted —CH₂—. In some embodiments, L⁴ is selected from —CH₂— and —CH(CH₃)—. In some embodiments, L⁴ is —CH₂—.

In some embodiments, L⁵ is an optionally substituted bivalent straight C₁₋₆ (e.g., C₁₋₃, C₁, C₂, C₃, C₄, C₅ or C₆) hydrocarbon chain. In some embodiments, L¹ is an optionally substituted bivalent branched C₁₋₆ (e.g., C₁₋₃, C₁, C₂, C₃, C₄, C₅ or C₆) hydrocarbon chain. In some embodiments of any Formulae described herein, L¹ is a covalent bond or an optionally substituted bivalent C₁₋₃ straight or branched hydrocarbon chain. In some embodiments, L¹ is a covalent bond. In some embodiments, L¹ is an optionally substituted bivalent Che straight or branched hydrocarbon chain. In some embodiments, L⁵ is a bivalent C₁₋₆ straight or branched hydrocarbon chain. In some embodiments, L¹ is an optionally substituted bivalent C₁₋₃ straight or branched hydrocarbon chain. In some embodiments, L¹ is a bivalent C₁₋₃ straight or branched hydrocarbon chain. In some embodiments, L⁵ is optionally substituted —CH₂—. In some embodiments, L⁵ is selected from —CH₂— and —CH(CH₃)—. In some embodiments, L⁵ is —CH₂—.

In some embodiments, R⁸ is —R as described herein. In some embodiments. R⁸ is —R but is not hydrogen. In some embodiments of any Formulae described herein, each R⁸ is independently hydrogen or optionally substituted C₁₋₆ alkyl. In some embodiments, R⁸ is hydrogen. In some embodiments, R⁸ is optionally substituted C₁₋₆ aliphatic. In some embodiments, R⁸ is optionally substituted C₁₋₆ alkyl. In some embodiments, R⁸ is C₁₋₆ alkyl. In some embodiments, R⁸ is optionally substituted C₁₋₂ alkyl. In some embodiments, R⁸ is C₁₋₂ alkyl (e.g., methyl). In some embodiments, R⁸ is optionally substituted C₃₋₆ cycloaliphatic. In some embodiments, R⁸ is optionally substituted C₃₋₆ cycloalkyl. In some embodiments, R⁸ is C₃₋₄ cycloalkyl (e.g., cyclopropyl). In some embodiments, R⁸ is —C(O)R. In some embodiments, R⁸ is —C(O)(C₁₋₆ alkyl). In some embodiments, R⁸ is —S(O)₂R. In some embodiments, R⁸ is —S(O)₂(C₁₋₆ alkyl). In some embodiments, the two R⁸ groups are the same. In some embodiments, they are different. In some embodiments, two R⁸ together with the nitrogen atom to which they are attached, combine to form an optionally substituted 3-16 (e.g., 3, 4, 5, 6, 7, 8, 9, 10, 11, 12, 13, 14, 15, 16, 3-15, 3-10, 5-10, 3-8, 3-7, or 5-6) membered ring having 1-5 (e.g., 1-2, 1-3, 1-4, 1, 2, 3, 4, or 5) heteroatoms independently selected from N, O, and S. In some embodiments, a formed ring is 3-10 membered. In some embodiments, a formed ring is 3-6 membered. In some embodiments, a formed ring is 3-membered. In some embodiments, it is 4-membered. In some embodiments, it is 5-membered. In some embodiments, it is 6-membered. In some embodiments, it is monocyclic. In some embodiments, it is bicyclic. In some embodiments, it is polycyclic. In some embodiments, it is saturated or at least one monocyclic unit is saturated. In some embodiments, it is partially unsaturated or at least one monocyclic unit is partially unsaturated. In some embodiments, it is aromatic or at least one monocyclic unit is aromatic. In some embodiments, it is heteroaromatic or at least one monocyclic unit is heteroaromatic.

### Linker

In some embodiments of any Formulae described herein, linker is a linking moiety (i.e., any suitable bivalent moiety that connects a PBM to a LBM). In some embodiments, linker is a covalent bond. In some embodiments, linker is an optionally substituted bivalent C₁-C₂₀ (e.g., C₁-C₁₅, C₁-C₁₀, C₁-C₆, C₁-C₄, C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀) hydrocarbon chain (which can be straight or branched, and saturated or partially unsaturated, and can comprise one or more (e.g., 1-2, 1-3, 1-4, 1, 2, 3, 4, or 5) hydrocarbon rings), wherein one or more (e.g., 1-10, 1-8, 1-6, 1-5, 1-3, 1-2, 2-10, 2-5, 2-3, 1, 2, 3, 4, 5, 6, 7, 8, 9 or 10) methylene units are optionally and independently replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —S(O)—, —SO₂—, —SO₂N(R)—, —N(R)SO₂—, or -Cy-. In some embodiments, various linkers described herein can provide superior properties, e.g., improved potency, improved half-life, improved stability, and/or decreased toxicity.

In some embodiments, linker is a particular length (e.g., as measured by number of atoms). It will be appreciated that when the length of linker is described, the longest contiguous chain of atoms is used. For example, in some embodiments, linker has the following structure, which is 14 atoms in length (counted as shown with italicized numbers):

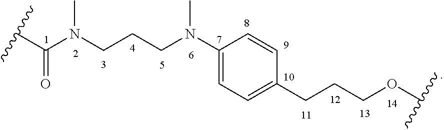 [SMILES: *OCCCc1ccc(N(C)CCCN(C)C(*)=O)cc1]

In some embodiments, a linker is 2-16 atoms in length. In some embodiments, a linker is 2-13 atoms in length. In some embodiments, a linker is 2-10 atoms in length. In some embodiments, a linker is 2-8 atoms in length. In some embodiments, a linker is 2-7 atoms in length. In some embodiments, a linker is 0-16 atoms in length. In some embodiments, a linker is 0-13 atoms in length. In some embodiments, a linker is 0-10 atoms in length. In some embodiments, a linker is 0-7 atoms in length. In some embodiments, a linker is 4-16 atoms in length. In some embodiments, a linker is 4-13 atoms in length. In some embodiments, a linker is 4-10 atoms in length. In some embodiments, a linker is 4-8 atoms in length. In some embodiments, a linker is 4-7 atoms in length. In some embodiments, a linker is less than 14 atoms in length. In some embodiments, a linker is less than 11 atoms in length. In some embodiments, a linker is less than 9 atoms in length. In some embodiments, a linker is less than 8 atoms in length.

Alternative or additionally, in some embodiments, the shortest-path length of a linker (the number of atoms in the chain from one end of a linker to the other following the shorted path (the shortest path chain)) is about 0-16 (e.g., 0, 1, 2, 3, 4, 5, 6, 7, 8, 9, 10, 11, 12, 13, 14, 15, 16, 1-15, 1-10, 1-9, 1-8, 1-7, 1-6, 1-5, 3-15, 3-10, 5-10, 3-8, 3-7, or 5-6) atoms. In some embodiments, it is 0. In some embodiments, it is about 1-16 atoms. In some embodiments, it is about 1-10 atoms. In some embodiments, it is 1 atom. In some embodiments, it is about or no more than about 2 atoms. In some embodiments, it is about or no more than about 2 atoms. In some embodiments, it is about or no more than about 3 atoms. In some embodiments, it is about or no more than about 4 atoms. In some embodiments, it is about or no more than about 5 atoms. In some embodiments, it is about or no more than about 6 atoms. In some embodiments, it is about or no more than about 7 atoms. In some embodiments, it is about or no more than about 8 atoms. In some embodiments, it is about or no more than about 9 atoms. In some embodiments, it is about or no more than about 10 atoms. In some embodiments, it is about or no more than about 11 atoms. In some embodiments, it is about or no more than about 12 atoms. In some embodiments, it is about or no more than about 13 atoms. In some embodiments, it is about or no more than about 14 atoms.

In some embodiments of any Formulae described herein, linker is a covalent bond.

In some embodiments of any Formulae described herein, linker is an optionally substituted, bivalent, straight or branched, saturated or unsaturated C₁-C₂₀ hydrocarbon chain, wherein one or more methylene units are optionally and independently replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —SO₂—, —SON₂(R)—, —N(R)SO₂—, or -Cy-. In some embodiments, linker is an optionally substituted, bivalent, straight or branched, saturated or unsaturated C₁-C₂₀ hydrocarbon chain, wherein at least one methylene unit is replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —SO₂—, —SO₂N(R)—, —N(R)SO₂—, or -Cy-. In some embodiments, linker is an optionally substituted, bivalent, straight or branched, saturated or unsaturated C₁-C₂₀ hydrocarbon chain, wherein at least two methylene units are replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —SO₂—, —SO₂N(R)—, —N(R)SO₂—, or -Cy-.

In some embodiments, linker is an optionally substituted, bivalent, straight or branched, saturated or unsaturated C₁-C₁₀ hydrocarbon chain, wherein one or more methylene units are optionally and independently replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —SO₂—, —SO₂N(R)—, —N(R)SO₂—, or -Cy-. In some embodiments, linker is an optionally substituted, bivalent, straight or branched, saturated or unsaturated C₁-C₁₀ hydrocarbon chain, wherein at least one methylene unit is replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—,—N(R)C(O)S—, —SC(O)N(R)—, —N(R)C(NR)N(R)—, —SO₂—, —SO₂N(R)—, —N(R)SO₂—, or -Cy-. In some embodiments, linker is an optionally substituted, bivalent, straight or branched, saturated or unsaturated C₁-C₁₀ hydrocarbon chain, wherein at least two methylene units are replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —SO₂—, —SO₂N(R)—, —N(R)SO₂—, or -Cy-.

In some embodiments, linker is an optionally substituted, bivalent, straight or branched, saturated or unsaturated C₁-C₂₀ hydrocarbon chain, wherein one or more methylene units are optionally and independently replaced by —O—, —N(R)—, —C(O)—, —OC(O)—, —C(O)O—, —C(O)N(R)—, —N(R)C(O)—, or -Cy-. In some embodiments, linker is an optionally substituted, bivalent, straight or branched, saturated or unsaturated C₁-C₂₀ hydrocarbon chain, wherein at least one methylene unit is replaced by —O—, —N(R)—, —C(O)—, —OC(O)—, —C(O)O—, —C(O)N(R)—, —N(R)C(O)—, or -Cy-. In some embodiments, linker is an optionally substituted, bivalent, straight or branched, saturated or unsaturated C₁-C₂₀ hydrocarbon chain, wherein at least two methylene units are replaced by —O—, —N(R)—, —C(O)—, —OC(O)—, —C(O)O—, —C(O)N(R)—, —N(R)C(O)—, or -Cy-.

In some embodiments, linker is an optionally substituted, bivalent, straight or branched, saturated or unsaturated C₁-C₁₀ (e.g., C₁-C₈, C₁-C₆, C₁-C₅, C₁-C₄, C₁, C₂, C₃, C₄, C₅, C₆, C₇, C₈, C₉, or C₁₀) hydrocarbon chain, wherein one or more methylene units are optionally and independently replaced by —O—, —N(R)—, —C(O)—, —OC(O)—, —C(O)O—, —C(O)N(R)—, —N(R)C(O)—, or -Cy-. In some embodiments, linker is an optionally substituted, bivalent, straight or branched, saturated or unsaturated C₁-C₁₀ hydrocarbon chain, wherein at least one methylene unit is replaced by —O—, —N(R)—, —C(O)—, —OC(O)—, —C(O)O—, —C(O)N(R)—, —N(R)C(O)—, or -Cy-. In some embodiments, linker is an optionally substituted, bivalent, straight or branched, saturated or unsaturated C₁-C₁₀ hydrocarbon chain, wherein at least two methylene units are replaced by —O—, —N(R)—, —C(O)—, —OC(O)—, —C(O)O—, —C(O)N(R)—, —N(R)C(O)—, or -Cy-.

As exemplified herein, in various embodiments, linkers in provided compounds are short (e.g., with lengths and/or shortest path lengths about or no more than about 5, 6, 7, 8, 9, 10, 11, 12, 13, 14, or 15 atoms; in many cases about or no more than about 8, 9 or 10 atoms) and provide rigidity (e.g., with about or no more than about 0, 1, 2, 3, 4 or 5 non-ring consecutive sp³ atoms in the shortest path chain, with about or no more than about 0, 1, 2, 3, 4 or 5 non-ring consecutive atoms in the shortest path chain, with about or no more than about 0, 1, 2, 3, 4 or 5 non-ring sp³ atoms in the shortest path chain, with about or no more than about 0, 1, 2, 3, 4 or 5 non-ring atoms in the shortest path chain, with about or no more than about 0, 1, 2, 3, 4 or 5 non-ring consecutive sp³ atoms in the shortest path chain each atom of which is independently C, N, O or S, with about or no more than about 0, 1, 2, 3, 4 or 5 non-ring consecutive atoms in the shortest path chain each atom of which is independently C, N, O or S, with about or no more than about 0, 1, 2, 3, 4 or 5 non-ring sp³ C, N, O or S atoms in the shortest path chain, or with about or no more than about 0, 1, 2, 3, 4 or 5 non-ring C, N, O or S atoms in the shortest path chain). In some embodiments, the number of non-ring consecutive sp³ atoms in the shortest path chain of a linker is about or no more than about 5. In some embodiments, the number of non-ring consecutive sp³ atoms in the shortest path chain of a linker is about or no more than about 4. In some embodiments, the number of non-ring consecutive sp³ atoms in the shortest path chain of a linker is about or no more than about 3. In some embodiments, the number of non-ring consecutive sp³ atoms in the shortest path chain of a linker is about or no more than about 2. In some embodiments, the number of non-ring sp³ atoms in the shortest path chain of a linker is about or no more than about 5. In some embodiments, the number of non-ring sp³ atoms in the shortest path chain of a linker is about or no more than about 4. In some embodiments, the number of non-ring sp³ atoms in the shortest path chain of a linker is about or no more than about 3. In some embodiments, the number of non-ring sp³ atoms in the shortest path chain of a linker is about or no more than about 2. In some embodiments, the number of non-ring sp³ atoms in the shortest path chain of a linker is about or no more than about 1. In some embodiments, the number of non-ring sp³ atoms in the shortest path chain of a linker is 0. In some embodiments, the number of non-ring sp³ C, O, and S atoms in the shortest path chain of a linker is about or no more than about 5. In some embodiments, the number of non-ring sp³ C, O, and S atoms in the shortest path chain of a linker is about or no more than about 4. In some embodiments, the number of non-ring sp³ C, O, and S atoms in the shortest path chain of a linker is about or no more than about 3. In some embodiments, the number of non-ring sp³ C, O, and S atoms in the shortest path chain of a linker is about or no more than about 2. In some embodiments, the number of non-ring sp³ C, O, and S atoms in the shortest path chain of a linker is about or no more than about 1. In some embodiments, the number of non-ring sp³ C, O, and S atoms in the shortest path chain of a linker is 0. In some embodiments, the number of non-ring C, O, and S atoms in the shortest path chain of a linker is about or no more than about 5. In some embodiments, the number of non-ring C, O, and S atoms in the shortest path chain of a linker is about or no more than about 4. In some embodiments, the number of non-ring C, O, and S atoms in the shortest path chain of a linker is about or no more than about 3. In some embodiments, the number of non-ring C, O, and S atoms in the shortest path chain of a linker is about or no more than about 2. In some embodiments, the number of non-ring C, O, and S atoms in the shortest path chain of a linker is about or no more than about 1. In some embodiments, the number of non-ring atoms in the shortest path chain of a linker is 0. In some embodiments, the number of non-ring atoms in the shortest path chain of a linker is about or no more than about 5. In some embodiments, the number of non-ring atoms in the shortest path chain of a linker is about or no more than about 4. In some embodiments, the number of non-ring atoms in the shortest path chain of a linker is about or no more than about 3. In some embodiments, the number of non-ring atoms in the shortest path chain of a linker is about or no more than about 2. In some embodiments, the number of non-ring atoms in the shortest path chain of a linker is about or no more than about 1. In some embodiments, the number of non-ring atoms in the shortest path chain of a linker is 0. As those skilled in the art appreciate, there can be one or more shortest path chains of a linker. In some embodiments, a linker comprises one or more (e.g., 1-2, 1-3, 1-4, 1, 2, 3, 4, or 5) -Cy-. In some embodiments, a linker comprises one or more (e.g., 1-2, 1-3, 1-4, 1, 2, 3, 4, or 5) -Cy- in its shortest path chain. In some embodiments, each -Cy- is independently in the shortest path chain of a linker. In some embodiments, -Cy- is monocyclic. In some embodiments, -Cy- is bicyclic. In some embodiments, -Cy- is spiro-bicyclic. In some embodiments, -Cy- is polycyclic. In some embodiments, -Cy- is saturated. In some embodiments, -Cy- is partially unsaturated. In some embodiments, -Cy- is aromatic. In some embodiments, -Cy- is 3-10 (e.g., 3-8, 3-6, 3-5, 5-6, 3, 4, 5, 6, 7, 8, 9 or 10) membered and is monocyclic. In some embodiments, -Cy- is 6-16 (e.g., 6-15, 7-15, 10-15, 10-16, 11-16, 11-15, 10-11, 6, 7, 8, 9, 10, 11, 12, 13, 14, 15 or 16) membered and is bicyclic. In some embodiments, -Cy- is 6-16 (e.g., 6-15, 7-15, 10-15, 10-16, 11-16, 11-15, 10-11, 6, 7, 8, 9, 10, 11, 12, 13, 14, 15 or 16) membered and is spiro-bicyclic. In some embodiments, -Cy- is 10-12 membered and is bicyclic. In some embodiments, -Cy- is 10-12 membered and is spiro-bicyclic. In some embodiments, -Cy- is 11-membered and is bicyclic. In some embodiments, -Cy- is 11-membered and is spiro-bicyclic. In some embodiments, each monocyclic ring unit in -Cy- is independently 3-10 (e.g., 3-8, 3-6, 3-5, 5-6, 3, 4, 5, 6, 7, 8, 9 or 10) membered. In some embodiments, each monocyclic ring unit in -Cy- is independently 3-8 (e.g., 3-6, 3-5, 5-6, 3, 4, 5, 6, 7, 8, 9 or 10) membered. In some embodiments, each monocyclic ring unit in -Cy- is independently 3-6 (e.g., 3-5, 5-6, 3, 4, 5, 6, 7, 8, 9 or 10) membered. In some embodiments, each monocyclic ring unit in -Cy- is independently 5- or 6-membered. In some embodiments, a monocyclic ring unit in -Cy- is saturated. In some embodiments, a monocyclic ring unit in -Cy- is partially unsaturated. In some embodiments, a monocyclic ring unit in -Cy- is aromatic. In some embodiments, each monocyclic ring unit in -Cy- is independently saturated or partially unsaturated. In some embodiments, each monocyclic ring unit in -Cy- is saturated. In some embodiments, the number of N, O or S heteroatoms in each monocyclic ring unit in -Cy- is independently 0-4 (e.g., 0, 1-4, 1-2, 1, 2, 3, or 4). In some embodiments, the number of N, O or S heteroatoms in each monocyclic ring unit in -Cy- is independently 0-2. In some embodiments, the number of N, O or S heteroatoms in each monocyclic ring unit in -Cy- is independently 0-1. In some embodiments, -Cy-comprises at least one nitrogen. In some embodiments, each heteroatom in -Cy- is nitrogen. In some embodiments, it was observed that spirocyclic rings in linkers can provide various benefits and advantages including high potency. In some embodiments, the number of basic nitrogen atoms can be utilized to modulate compound properties and/or potency, e.g., pharmacokinetic properties. In some embodiments, it was observed that basic nitrogen atoms in linkers, e.g., in -Cy- (e.g., in piperidine and/or piperazine rings) can provide improved solubility, improved dissolution rate, and/or longer T_{1/2}, e.g., when assessed in rodent PK assays. Various embodiments of linkers are described herein as examples, e.g., below or in various compounds.

In some embodiments, a linker is an optionally substituted bivalent C₁-C₅ (e.g., C₁-C₄, C₁-C₃, C₁-C₂, C₁, C₂, C₃, C₄, or C₅) hydrocarbon chain (which can be straight or branched, and saturated or partially unsaturated), wherein one or more (e.g., 1, 2, 3, 4, or 5) methylene units are optionally and independently replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —S(O)—, —SO₂—, —SO₂N(R)—, —N(R)SO₂—, or -Cy-. In some embodiments, a linker is an optionally substituted bivalent C₁-C₃ (e.g., C₁-C₂, C₁, C₂, or C₃) hydrocarbon chain (which can be straight or branched, and saturated or partially unsaturated), wherein one or more (e.g., 1, 2, 3, 4, or 5) methylene units are optionally and independently replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —S(O)—, —SO₂—, —SO₂N(R)—, —N(R)SO₂—, or -Cy-. In some embodiments, a linker is an optionally substituted bivalent straight C₁-C₃ (e.g., C₁-C₂, C₁, C₂, or C₃) hydrocarbon chain, wherein one or more (e.g., 1, 2, 3, 4, or 5) methylene units are optionally and independently replaced by —O—, —S—, —N(R)—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —S(O)—, —SO₂—, —SO₂N(R)—, —N(R)SO₂—, or -Cy-. In some embodiments, a linker is an optionally substituted bivalent straight C₁-C₃ (e.g., C₁-C₂, C₁, C₂, or C₃) alkylene chain, wherein one or more (e.g., 1, 2, 3, 4, or 5) methylene units are optionally and independently replaced by —O—, —S—, —N(R)—, —C(O)—, —C(S)—, —C(NR)—, or -Cy-. In some embodiments, a linker is an optionally substituted bivalent straight C₁ or C₂ alkylene, wherein one or more (e.g., 1, 2, 3, 4, or 5) methylene units are optionally and independently replaced by —O—, —S—, —N(R)—, —C(O)—, —C(S)—, —C(NR)—, or -Cy-. In some embodiments, a linker further has limited number of non-ring atoms in its shortest path chain as described herein, e.g., the number of non-ring atoms in its shortest path chain is about or no more than about 0, 1, 2, 3, 4 or 5 (as illustrated herein, in various embodiments, about or no more than about 2, and in various embodiments, about or no more than about 1). For example, in some embodiments, a linker is —C(O)-Cy-, wherein —C(O)— is bonded to PBM; in some embodiments, a linker is —CH₂-Cy-, wherein —CH₂— is bonded to PBM; in some embodiments, a linker is —CH₂—O—, wherein —CH₂— is bonded to PBM; in some embodiments, a linker is-Cy-O—, wherein -Cy- is bonded to PBM.

In some embodiments, about or no more than about 1-10 (e.g., 1-10, 1-8, 1-6, 1-5, 1-3, 1-2, 2-10, 2-5, 2-3, 1, 2, 3, 4, 5, 6, 7, 8, 9 or 10) methylene units are independently replaced as described herein. In some embodiments, about or no more than about 5 methylene units are independently replaced as described herein. In some embodiments, about or no more than about 4 methylene units are independently replaced as described herein. In some embodiments, about or no more than about 3 methylene units are independently replaced as described herein. In some embodiments, about or no more than about 2 methylene units are independently replaced as described herein. In some embodiments, about or no more than about 1 methylene unit is replaced as described herein.

In some embodiments, linker comprises an ether moiety (e.g., —O—). In some embodiments, linker is an optionally substituted, bivalent, straight or branched, saturated or unsaturated C₁-C₂₀ hydrocarbon chain, wherein at least one methylene unit is replaced by —O—. In some embodiments, linker is an optionally substituted, bivalent, straight or branched, saturated or unsaturated C₁-C₁₀ hydrocarbon chain, wherein at least one methylene unit is replaced by —O—.

In some embodiments, linker comprises an amine moiety (e.g., —N(R)—). In some embodiments, linker is an optionally substituted, bivalent, straight or branched, saturated or unsaturated C₁-C₂₀ hydrocarbon chain, wherein at least one methylene unit is replaced by —N(R)— (e.g., —NH— or —N(C₁₋₆alkyl)-). In some embodiments, linker is an optionally substituted, bivalent, straight or branched, saturated or unsaturated C₁-C₁₀ hydrocarbon chain, wherein at least one methylene unit is replaced by —N(R)— (e.g., —NH— or —N(C)₁₋₆alkyl)-). In some embodiments, a methylene unit is replaced by —NH—.

In some embodiments, linker comprises a carbonyl moiety. In some embodiments, linker is an optionally substituted, bivalent, straight or branched, saturated or unsaturated C₁-C₂₀ hydrocarbon chain, wherein at least one methylene unit is replaced by —C(O)—. In some embodiments, linker is an optionally substituted, bivalent, straight or branched, saturated or unsaturated C₁-C₁₀ hydrocarbon chain, wherein at least one methylene unit is replaced by —C(O)—.

In some embodiments, linker comprises an ester moiety. In some embodiments, linker is an optionally substituted, bivalent, straight or branched, saturated or unsaturated C₁-C₂₀ hydrocarbon chain, wherein at least one methylene unit is replaced by —OC(O)— or —C(O)O—. In some embodiments, linker is an optionally substituted, bivalent, straight or branched, saturated or unsaturated C₁-C₁₀ hydrocarbon chain, wherein at least one methylene unit is replaced by —OC(O)— or —C(O)O—.

In some embodiments, linker comprises an amide moiety. In some embodiments, linker is an optionally substituted, bivalent, straight or branched, saturated or unsaturated C₁-C₂₀ hydrocarbon chain, wherein at least one methylene unit is replaced by —C(O)N(R)— (e.g., —C(O)NH— or —C(O)N(C₁₋₆ alkyl)-). In some embodiments, linker is an optionally substituted, bivalent, straight or branched, saturated or unsaturated C₁-C₁₀ hydrocarbon chain, wherein at least one methylene unit is replaced by —C(O)N(R)— (e.g., —C(O)NH— or —C(O)N(C₁₋₆ alkyl)-). In some embodiments, linker is an optionally substituted, bivalent, straight or branched, saturated or unsaturated C₁-C₂₀ hydrocarbon chain, wherein at least one methylene unit is replaced by —N(R)C(O)— (e.g., —N(H)C(O)— or —N(C₁₋₆ alkyl)C(O)—). In some embodiments, linker is an optionally substituted, bivalent, straight or branched, saturated or unsaturated C₁-C₁₀ hydrocarbon chain, wherein at least one methylene unit is replaced by —N(R)C(O)— (e.g., —N(H)C(O)— or —N(C₁₋₆ alkyl)C(O)—). In some embodiments, In some embodiments, a methylene unit is replaced by —C(O)NH—.

In some embodiments, linker comprises a bivalent ring moiety (e.g., -Cy-). In some embodiments, linker is an optionally substituted, bivalent, straight or branched, saturated or unsaturated C₁-C₂₀ hydrocarbon chain, wherein at least one methylene unit is replaced by -Cy-. In some embodiments, linker is an optionally substituted, bivalent, straight or branched, saturated or unsaturated C₁-C₁₀ hydrocarbon chain, wherein at least one methylene unit is replaced by -Cy-. In some such embodiments, Cy is not phenyl.

In some embodiments, linker comprises a triple bond. In some embodiments, linker is an optionally substituted, bivalent, straight or branched, partially unsaturated C₁-C₂₀ hydrocarbon chain comprising at least one triple bond, wherein one or more methylene units are optionally and independently replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —SO₂—, —SO₂N(R)—, —N(R)SO₂—, or -Cy-, In some embodiments, linker is an optionally substituted, bivalent, straight or branched, partially unsaturated C₁-C₁₀ hydrocarbon chain comprising at least one triple bond, wherein one or more methylene units are optionally and independently replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —SO₂—, —SO₂N(R)—, —N(R)SO₂—, or -Cy-. In some embodiments, linker is an optionally substituted, bivalent, straight or branched, partially unsaturated C₁-C₂₀ hydrocarbon chain comprising at least one triple bond, wherein one or more methylene units are optionally and independently replaced by —O—, —N(R)—, —C(O)—, —OC(O)—, —C(O)O—, —C(O)N(R)—, —N(R)C(O)—, or -Cy-. In some embodiments, linker is an optionally substituted, bivalent, straight or branched, partially unsaturated C₁-C₁₀ hydrocarbon chain comprising at least one triple bond, wherein one or more methylene units are optionally and independently replaced by —O—, —N(R)—, —C(O)—, —OC(O)—, —C(O)O—, —C(O)N(R)—, —N(R)C(O)—, or -Cy-.

In some embodiments of any Formulae described herein, linker has the following structure:

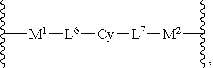

wherein Cy is as defined herein for Formula II and described in classes and subclasses herein, both singly and in combination; and:
M¹ and M² are each independently absent, —CH₂—, —O—, —N(R)—, —C(O)—, —OC(O)—, —C(O)O—, —C(O)N(R)—, or —N(R)C(O)—; and
L⁶ and L⁷ are each independently a covalent bond or an optionally substituted, bivalent, straight or branched, saturated or unsaturated C₁-C₁₀ hydrocarbon chain, wherein one or more methylene units are optionally and independently replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —SO₂—, —SO₂N(R)—, —N(R)SO₂—, or -Cy-.

In some embodiments of any Formulae described herein, linker has the following structure:

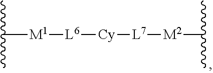

wherein Cy is as defined herein for Formula II and described in classes and subclasses herein, both singly and in combination; and:

M¹ and M² are each independently absent, —CH₂—, —O—, —N(R)—, —C(O)—, —OC(O)—, —C(O)O—, —C(O)N(R)—, or —N(R)C(O)—; and
L⁶ and L⁷ are each independently a covalent bond or an optionally substituted, bivalent, straight or branched, saturated or unsaturated C₁-C₅ (e.g., C₁-C₄, C₁-C₃, C₁-C₂, C₂-C₄, C₁, C₂, C₃, C₄, or C₅) hydrocarbon chain, wherein one or more methylene units are optionally and independently replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —SO₂—, —SO₂N(R)—, —N(R)SO₂—, or -Cy-.

In some embodiments of any Formulae described herein, linker is selected from:

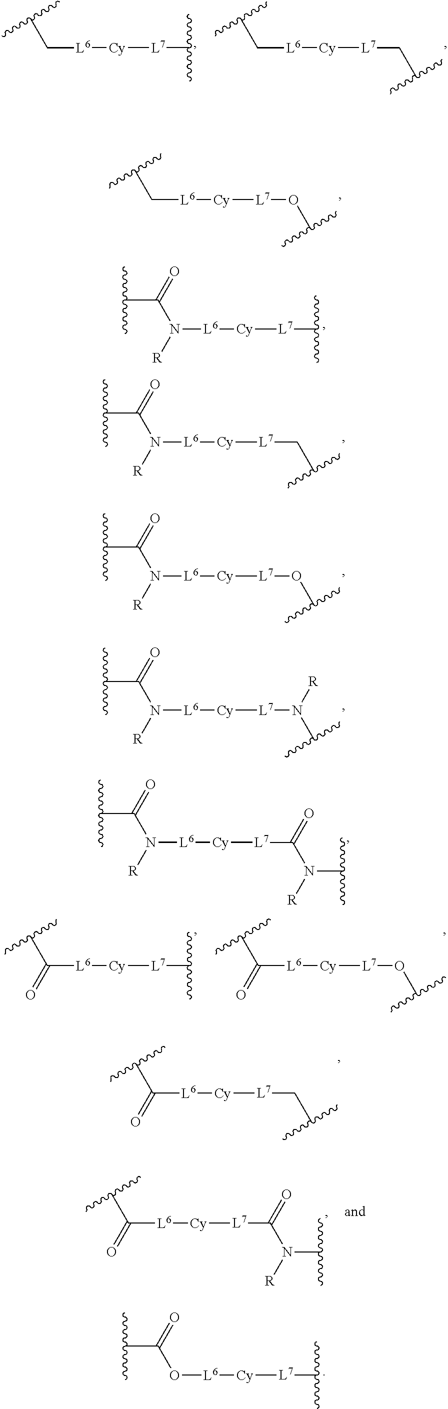

In some embodiments of any Formulae described herein, linker is

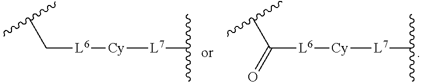

In some embodiments of any Formulae described herein, linker is selected from:

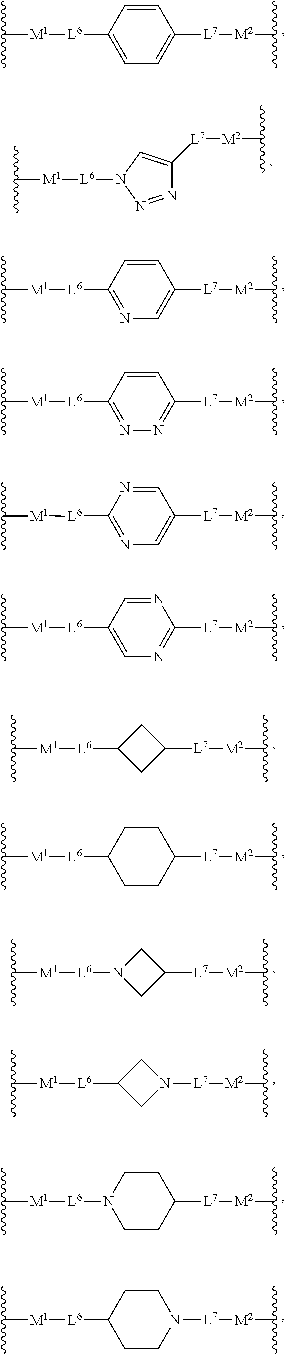

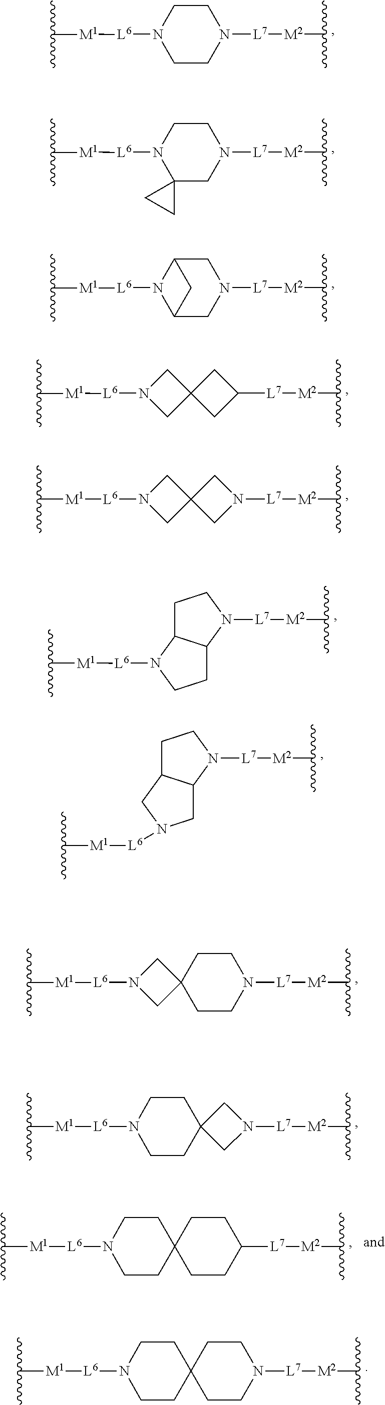

In some embodiments of any Formulae described herein, linker has the following structure:

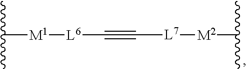

wherein:
M¹ and M² are each independently absent, —CH₂—, —O—, —N(R)—, —C(O)—, —OC(O)—, —C(O)O—, —C(O)N(R)—, or —N(R)C(O)—; and
L⁶ and L⁷ are each independently a covalent bond or an optionally substituted, bivalent, straight or branched, saturated or unsaturated C₁-C₁₀ hydrocarbon chain, wherein one or more methylene units are optionally and independently replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —SO₂—, —SO₂N(R)—, —N(R)SO₂—, or -Cy-.

In some embodiments of any Formulae described herein, linker is selected from:

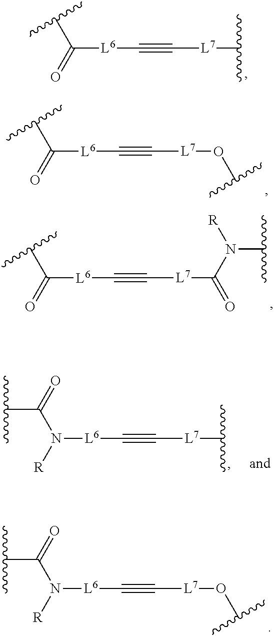

In some embodiments of any Formulae described herein, linker has the following structure:

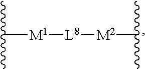

wherein:
M¹ and M² are each independently absent, —CH₂—, —O—, —N(R)—, —C(O)—, —OC(O)—, —C(O)O—, —C(O)N(R)—, or —N(R)C(O)—; and
L⁸ is a covalent bond or an optionally substituted, bivalent, straight or branched, saturated or unsaturated C₁-C₁₅ hydrocarbon chain, wherein one or more methylene units are optionally and independently replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —SO₂—, —SO₂N(R)—, —N(R)SO₂—, or -Cy-.

In some embodiments of any Formulae described herein, linker is selected from:

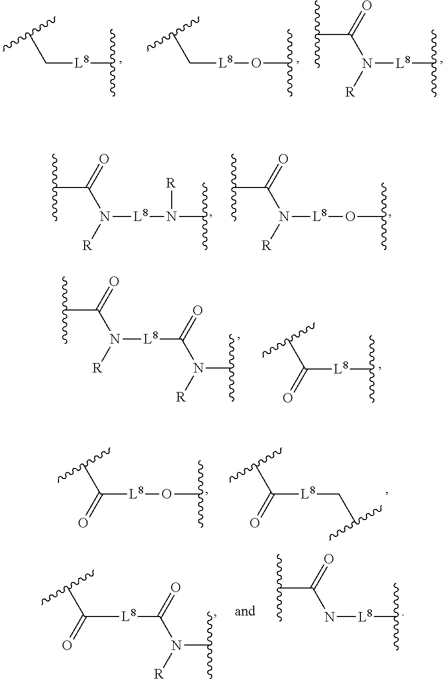

In some embodiments, linker is -M¹-L⁶-L⁷-M².

In some embodiments of any Formulae described herein, M¹ is —CH₂—, —O—, —N(R)—, —C(O)—, —OC(O)—, —C(O)O—, —C(O)N(R)—, or —N(R)C(O)—. In some embodiments, M¹ is absent. In some embodiments. M¹ is optionally substituted —CH₂—. In some embodiments, M¹ is —CH₂—. In some embodiments, M¹ is —O—. In some embodiments, M¹ is —N(R)— (e.g., —N(H)— or —N(CH₃)—). In some embodiments, M¹ is —C(O)—. In some embodiments, M¹ is —OC(O)—. In some embodiments, M¹ is —C(O)O—. In some embodiments, M¹ is —C(O)N(R)— (e.g., —C(O)N(H)— or —C(O)N(CH₃)—). In some embodiments, M¹ is —N(R)C(O)— (e.g., —N(H)C(O)— or —N(CH₃)C(O)—). In some embodiments, a variable is a covalent bond, and as those of ordinary skill in the art appreciate, it can be properly considered as absent. In some embodiments, M¹ is bonded to PBM.

In some embodiments of any Formulae described herein, M² is —CH₂—, —O—, —N(R)—, —C(O)—, —OC(O)—, —C(O)O—, —C(O)N(R)—, or —N(R)C(O)—. In some embodiments, M² is absent. In some embodiments, M² is —CH₂—. In some embodiments. M² is —O—. In some embodiments, M² is —N(R)— (e.g., —N(H)— or —N(CH₃)—). In some embodiments, M² is —C(O)—. In some embodiments, M² is —OC(O)—. In some embodiments, M² is —C(O)O—. In some embodiments, M² is —C(O)N(R)— (e.g., —C(O)N(H)— or —C(O)N(CH₃)—. In some embodiments, M² is —N(R)C(O)— (e.g., —N(H)C(O)— or —N(CH₃)C(O)—). In some embodiments, M² is bonded to LBM.

In some embodiments of any Formulae described herein, L⁶ is a covalent bond.

In some embodiments, L⁶ is an optionally substituted, bivalent, straight or branched, saturated or unsaturated C₁-C₁₀ hydrocarbon chain, wherein one or more methylene units are optionally and independently replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —SO₂—, —SO₂N(R)—, —N(R)SO₂—, or -Cy-. In some embodiments, L⁶ is an optionally substituted, bivalent, straight or branched, saturated or unsaturated C₁-C₁₀ hydrocarbon chain, wherein at least one methylene unit is replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —SO₂—, —SO₂N(R)—, —N(R)SO₂—, or -Cy-. In some embodiments, L⁶ is an optionally substituted, bivalent, straight or branched, saturated or unsaturated C₁-C₁₀ hydrocarbon chain, wherein at least two methylene units are replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —SO₂—, —SO₂N(R)—, —N(R)SO₂—, or -Cy-.

In some embodiments, L⁶ is an optionally substituted, bivalent, straight or branched, saturated or unsaturated C₁-C₁₀ hydrocarbon chain, wherein one or more methylene units are optionally and independently replaced by —O—, —N(R)—, —C(O)—, —OC(O)—, —C(O)O—, —C(O)N(R)—, —N(R)C(O)—, or -Cy-. In some embodiments, L⁶ is an optionally substituted, bivalent, straight or branched, saturated or unsaturated C₁-C₁₀ hydrocarbon chain, wherein at least one methylene unit is replaced by —O—, —N(R)—, —C(O)—, —OC(O)—, —C(O)O—, —C(O)N(R)—, —N(R)C(O)—, or -Cy-, In some embodiments, L⁶ is an optionally substituted, bivalent, straight or branched, saturated or unsaturated C₁-C₁₀ hydrocarbon chain, wherein at least two methylene units are replaced by —O—, —N(R)—, —C(O)—, —OC(O)—, —C(O)O—, —C(O)N(R)—, —N(R)C(O)—, or -Cy-.

In some embodiments, L⁶ is an optionally substituted, bivalent, straight or branched, saturated or unsaturated C₁-C₁₀ hydrocarbon chain, wherein one or more methylene units are optionally and independently replaced by —O—, —N(R)—, —C(O)—, —OC(O)—, —C(O)O—, —C(O)N(R)—, or —N(R)C(O)—. In some embodiments, L⁶ is an optionally substituted, bivalent, straight or branched, saturated or unsaturated C₁-C₁₀ hydrocarbon chain, wherein at least one methylene unit is replaced by —O—, —N(R)—, —C(O)—, —OC(O)—, —C(O)O—, —C(O)N(R)—, or —N(R)C(O)—. In some embodiments, L⁶ is an optionally substituted, bivalent, straight or branched, saturated or unsaturated C₁-C₁₀ hydrocarbon chain, wherein at least two methylene units are replaced by —O—, —N(R)—, —C(O)—, —OC(O)—, —C(O)O—, —C(O)N(R)—, or —N(R)C(O)—.

In some embodiments, L⁶ is an optionally substituted, bivalent, straight or branched, saturated or unsaturated C₁-C₁₀ hydrocarbon chain, wherein one or more methylene units are optionally and independently replaced by —O—. In some embodiments, L⁶ is an optionally substituted, bivalent, straight or branched, saturated or unsaturated C₁-C₁₀ hydrocarbon chain, wherein at least one methylene unit is replaced by —O—. In some embodiments, L⁶ is an optionally substituted, bivalent, straight or branched, saturated or unsaturated C₁-C₁₀ hydrocarbon chain, wherein at least two methylene units are replaced by —O—.

In some embodiments, L⁶ is an optionally substituted, bivalent, straight or branched, saturated or unsaturated C₁-C₁₀ hydrocarbon chain. In some embodiments. L⁶ is an optionally substituted, bivalent, straight or branched, saturated C₁-C₁₀ hydrocarbon chain. In some embodiments, L⁶ is an optionally substituted, bivalent, straight or branched, saturated or unsaturated C₁-C₆ hydrocarbon chain. In some embodiments, L⁶ is an optionally substituted, bivalent, straight or branched, saturated C₁-C₆ hydrocarbon chain.

In some embodiments, L⁶ is optionally substituted —CH₂—. In some embodiments, L⁶ is —CH₂—. In some embodiments, L⁶ is —C(O)—. In some embodiments, L⁶ is bonded to PBM.

In some embodiments of any Formulae described herein, L⁷ is a covalent bond.

In some embodiments. L⁷ is an optionally substituted, bivalent, straight or branched, saturated or unsaturated C₁-C₁₀ hydrocarbon chain, wherein one or more methylene units are optionally and independently replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —SO₂—, —SO₂N(R)—, —N(R)SO₂—, or -Cy-. In some embodiments, L⁷ is an optionally substituted, bivalent, straight or branched, saturated or unsaturated C₁-C₁₀ hydrocarbon chain, wherein at least one methylene unit is replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —SO₂—, —SO₂N(R)—, —N(R)SO₂—, or -Cy-. In some embodiments, L⁷ is an optionally substituted, bivalent, straight or branched, saturated or unsaturated C₁-C₁₀ hydrocarbon chain, wherein at least two methylene units are replaced by —O—, —S—, —N(R)—, —N═N—, —C(O)—, —C(S)—, —C(NR)—, —C(NOR)—, —C(NNR₂)—, —OC(O)—, —C(O)—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —SO₂—, —SO₂N(R)—, —N(R)SO₂—, or -Cy-.

In some embodiments, L⁷ is an optionally substituted, bivalent, straight or branched, saturated or unsaturated C₁-C₁₀ hydrocarbon chain, wherein one or more methylene units are optionally and independently replaced by —O—, —N(R)—, —C(O)—, —OC(O)—, —C(O)O—, —C(O)N(R)—, —N(R)C(O)—, or -Cy-. In some embodiments, L⁷ is an optionally substituted, bivalent, straight or branched, saturated or unsaturated C₁-C₁₀ hydrocarbon chain, wherein at least one methylene unit is replaced by —O—, —N(R)—, —C(O)—, —OC(O)—, —C(O)O—, —C(O)N(R)—, —N(R)C(O)—, or -Cy-. In some embodiments, L⁷ is an optionally substituted, bivalent, straight or branched, saturated or unsaturated C₁-C₁₀ hydrocarbon chain, wherein at least two methylene units are replaced by —O—, —N(R)—, —C(O)—, —OC(O)—, —C(O)O—, —C(O)N(R)—, —N(R)C(O)—, or -Cy-.

In some embodiments, L⁷ is an optionally substituted, bivalent, straight or branched, saturated or unsaturated C₁-C₁₀ hydrocarbon chain, wherein one or more methylene units are optionally and independently replaced by —O—, —N(R)—, —C(O)—, —OC(O)—, —C(O)O—, —C(O)N(R)—, or —N(R)C(O)—. In some embodiments, L⁷ is an optionally substituted, bivalent, straight or branched, saturated or unsaturated C₁-C₁₀ hydrocarbon chain, wherein at least one methylene unit is replaced by —O—, —N(R)—, —C(O)—, —OC(O)—, —C(O)O—, —C(O)N(R)—, or —N(R)C(O)—. In some embodiments, L⁷ is an optionally substituted, bivalent, straight or branched, saturated or unsaturated C₁-C₁₀ hydrocarbon chain, wherein at least two methylene units are replaced by —O—, —N(R)—, —C(O)—, —OC(O)—, —C(O)O—, —C(O)N(R)—, or —N(R)C(O)—.

In some embodiments, L⁷ is an optionally substituted, bivalent, straight or branched, saturated or unsaturated C₁-C₁₀ hydrocarbon chain, wherein one or more methylene units are optionally and independently replaced by —O—. In some embodiments, L⁷ is an optionally substituted, bivalent, straight or branched, saturated or unsaturated C₁-C₁₀ hydrocarbon chain, wherein at least one methylene unit is replaced by —O—. In some embodiments, L⁷ is an optionally substituted, bivalent, straight or branched, saturated or unsaturated C₁-C₁₀ hydrocarbon chain, wherein at least two methylene units are replaced by —O—.

In some embodiments, L⁷ is an optionally substituted, bivalent, straight or branched, saturated or unsaturated C₁-C₁₀ hydrocarbon chain. In some embodiments, L⁷ is an optionally substituted, bivalent, straight or branched, saturated C₁-C₁₀ hydrocarbon chain. In some embodiments, L⁷ is an optionally substituted, bivalent, straight or branched, saturated or unsaturated C₁-C₆ hydrocarbon chain. In some embodiments, L⁷ is an optionally substituted, bivalent, straight or branched, saturated C₁-C₆ hydrocarbon chain.

In some embodiments, L⁷ is bonded to LBM.

In some embodiments of any Formulae described herein, both L⁶ and L⁷ are a covalent bond.

In some embodiments of any Formulae described herein, L⁸ is a covalent bond.

In some embodiments, L⁸ is an optionally substituted, bivalent, straight or branched, saturated or unsaturated C₁-C₁₅ hydrocarbon chain, wherein one or more methylene units are optionally and independently replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —SO₂—, —SO₂N(R)—, —N(R)SO₂—, or -Cy-. In some embodiments, L⁸ is an optionally substituted, bivalent, straight or branched, saturated or unsaturated C₁-C₁₅ hydrocarbon chain, wherein at least one methylene unit is replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —SO₂—, —SO₂N(R)—, —N(R)SO₂—, or -Cy-. In some embodiments, L⁸ is an optionally substituted, bivalent, straight or branched, saturated or unsaturated C₁-C₁₅ hydrocarbon chain, wherein at least two methylene units are replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —SO₂—, —SO₂N(R)—, —N(R)SO₂—, or -Cy-.

In some embodiments, L⁸ is an optionally substituted, bivalent, straight or branched, saturated or unsaturated C₁-C₁₅ hydrocarbon chain, wherein one or more methylene units are optionally and independently replaced by —O—, —N(R)—, —C(O)—, —OC(O)—, —C(O)O—, —C(O)N(R)—, —N(R)C(O)—, or -Cy-. In some embodiments, L⁸ is an optionally substituted, bivalent, straight or branched, saturated or unsaturated C₁-C₁₅ hydrocarbon chain, wherein at least one methylene unit is replaced by —O—, —N(R)—, —C(O)—, —OC(O)—, —C(O)O—, —C(O)N(R)—, —N(R)C(O)—, or -Cy-. In some embodiments. L⁸ is an optionally substituted, bivalent, straight or branched, saturated or unsaturated C₁-C₁₅ hydrocarbon chain, wherein at least two methylene units are replaced by —O—, —N(R)—, —C(O)—, —OC(O)—, —C(O)O—, —C(O)N(R)—, —N(R)C(O)—, or -Cy-.

In some embodiments, L⁸ is an optionally substituted, bivalent, straight or branched, saturated or unsaturated C₁-C₁₅ hydrocarbon chain, wherein one or more methylene units are optionally and independently replaced by —O—, —N(R)—, —C(O)—, —OC(O)—, —C(O)O—, —C(O)N(R)—, or —N(R)C(O)—. In some embodiments, L⁸ is an optionally substituted, bivalent, straight or branched, saturated or unsaturated C₁-C₁₅ hydrocarbon chain, wherein at least one methylene unit is replaced by —O—, —N(R)—, —C(O)—, —OC(O)—, —C(O)O—, —C(O)N(R)—, or —N(R)C(O)—. In some embodiments, L⁸ is an optionally substituted, bivalent, straight or branched, saturated or unsaturated C₁-C₁₅ hydrocarbon chain, wherein at least two methylene units are replaced by —O—, —N(R)—, —C(O)—, —OC(O)—, —C(O)O—, —C(O)N(R)—, or —N(R)C(O)—.

In some embodiments, L⁸ is an optionally substituted, bivalent, straight or branched, saturated or unsaturated C₁-C₁₅ hydrocarbon chain, wherein one or more methylene units are optionally and independently replaced by —O—. In some embodiments. L⁸ is an optionally substituted, bivalent, straight or branched, saturated or unsaturated C₁-C₁₅ hydrocarbon chain, wherein at least one methylene unit is replaced by —O—. In some embodiments, L⁸ is an optionally substituted, bivalent, straight or branched, saturated or unsaturated C₁-C₁₅ hydrocarbon chain, wherein at least two methylene units are replaced by —O—.

In some embodiments, L⁸ is an optionally substituted, bivalent, straight or branched, saturated or unsaturated C₁-C₁₅ hydrocarbon chain. In some embodiments. L⁸ is an optionally substituted, bivalent, straight or branched, saturated C₁-C₁₅ hydrocarbon chain. In some embodiments, L⁸ is an optionally substituted, bivalent, straight or branched, saturated or unsaturated C₁-C₆ hydrocarbon chain. In some embodiments, L⁸ is an optionally substituted, bivalent, straight or branched, saturated C₁-C₆ hydrocarbon chain.

In some embodiments of any Formulae described herein, linker is selected from:
—CH₂—,

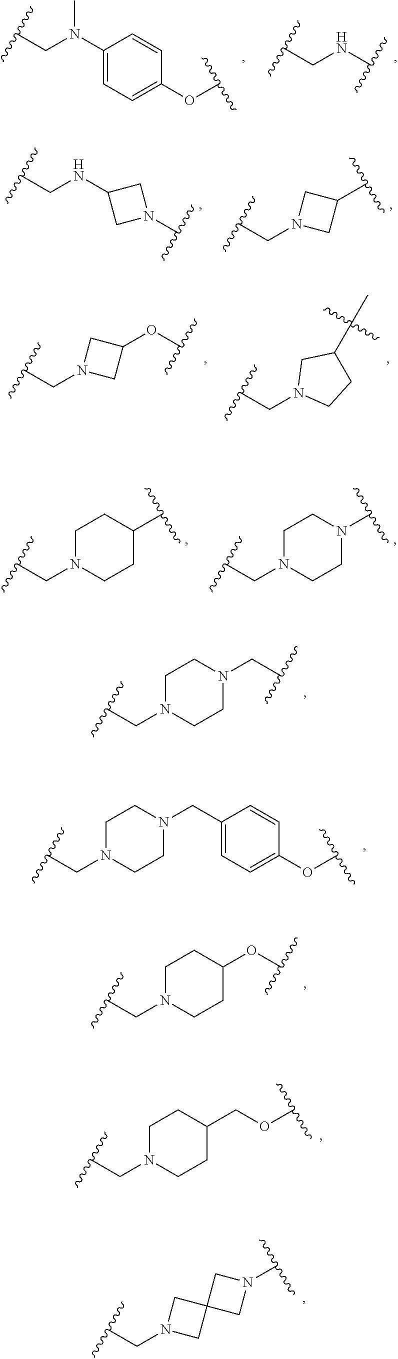

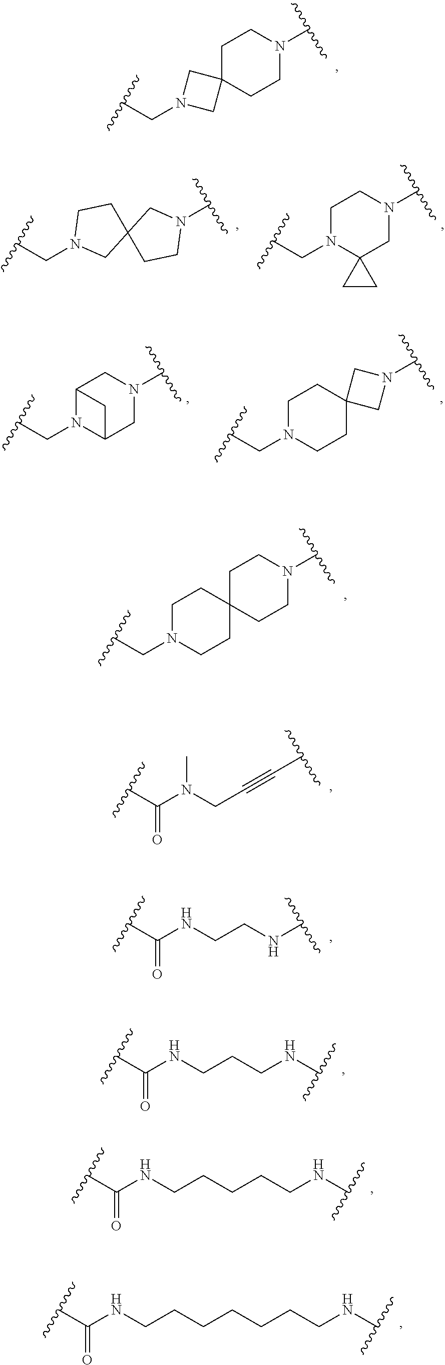

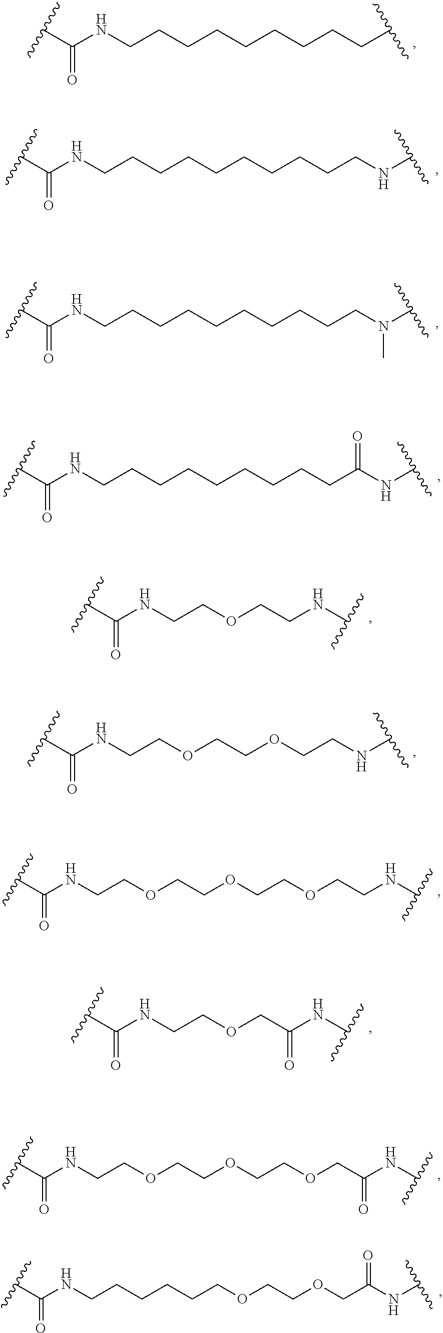

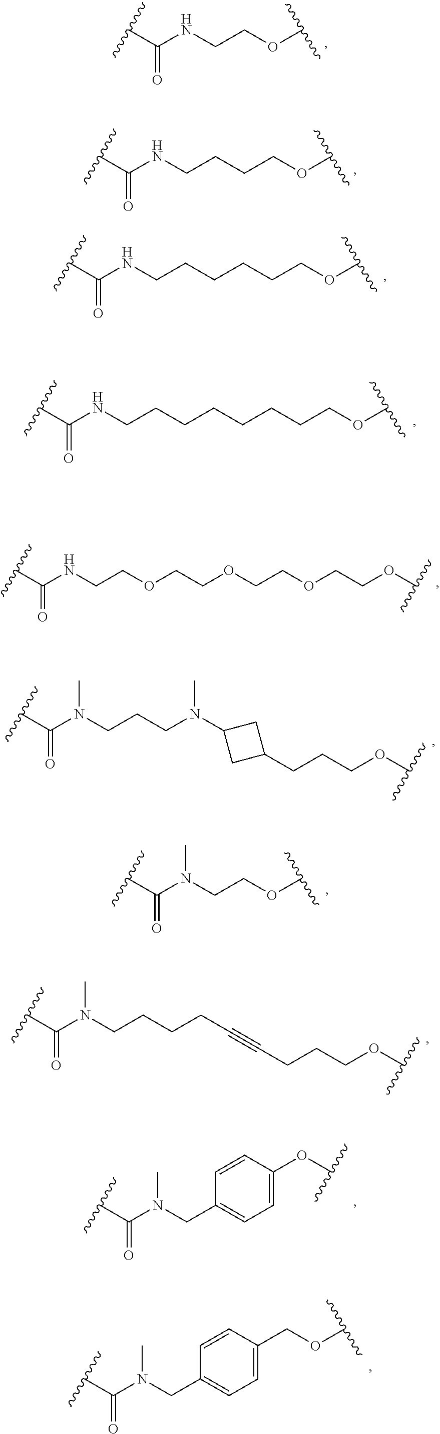

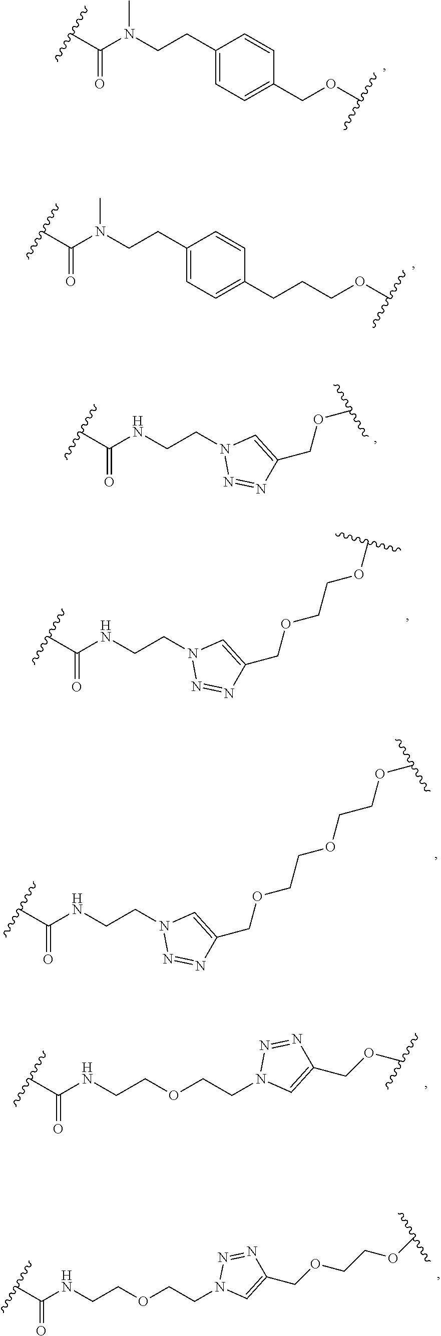

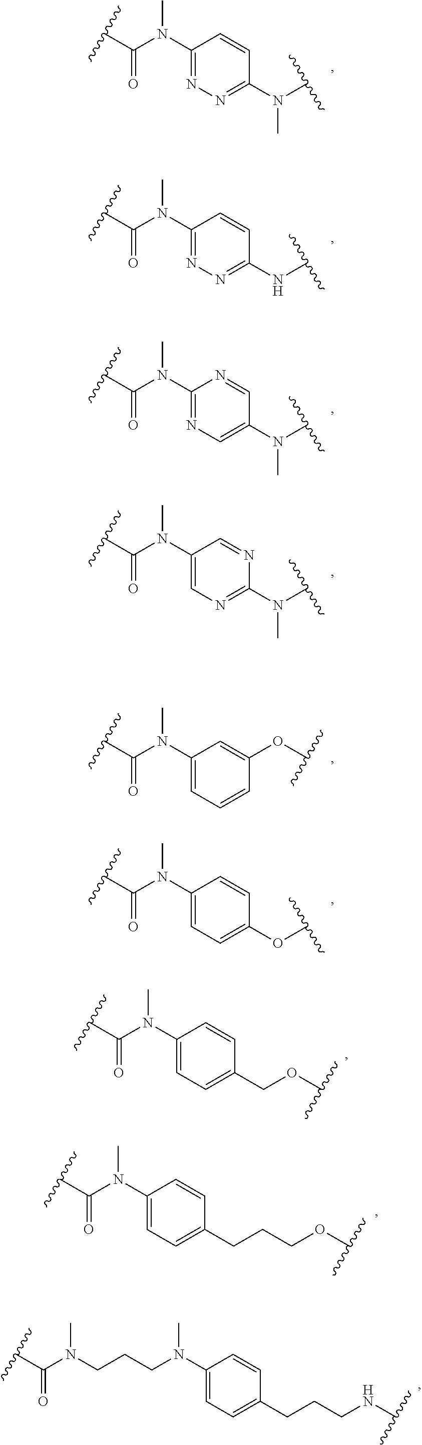

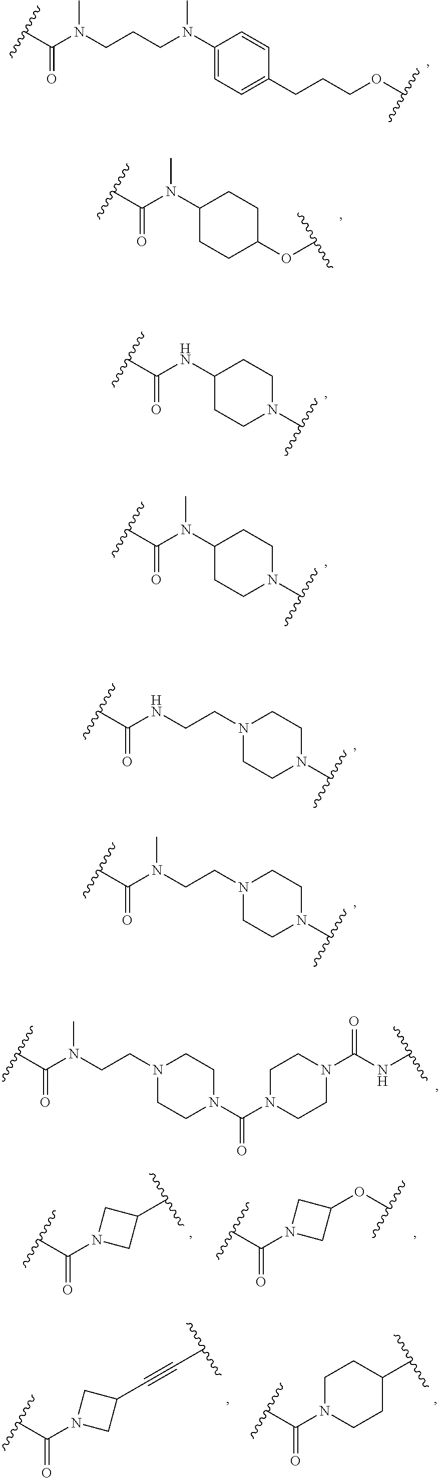

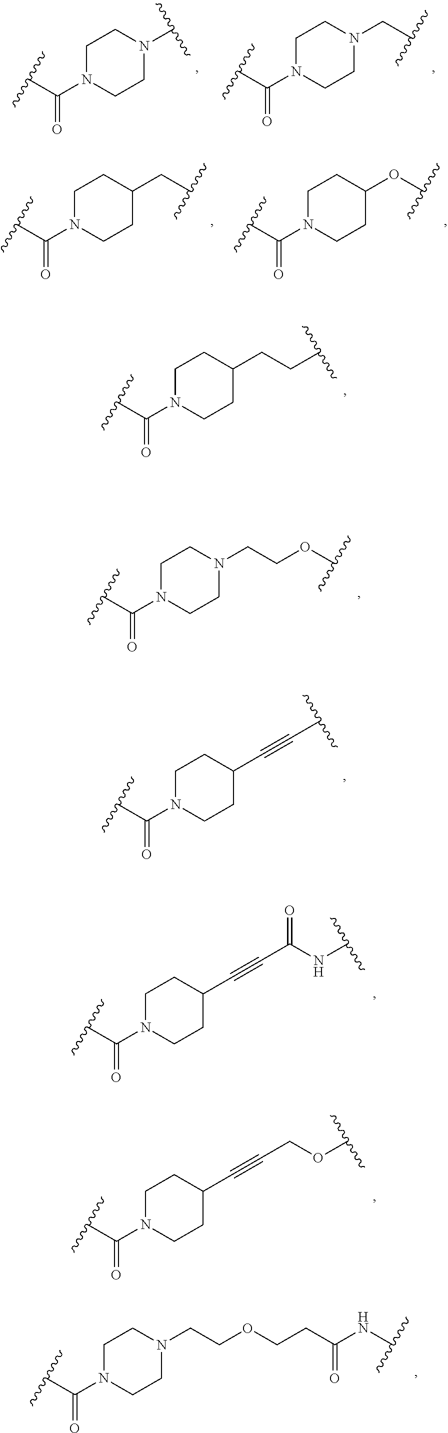

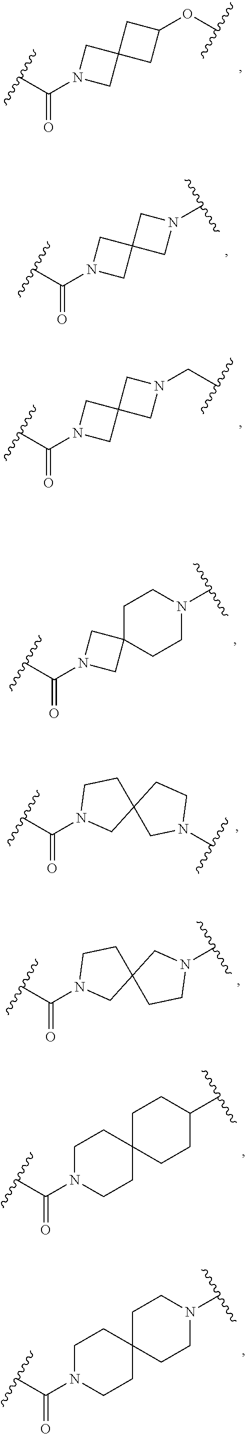

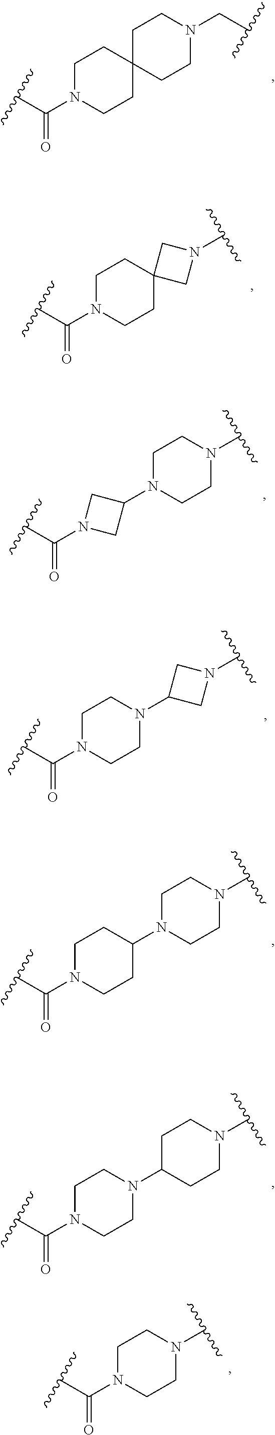

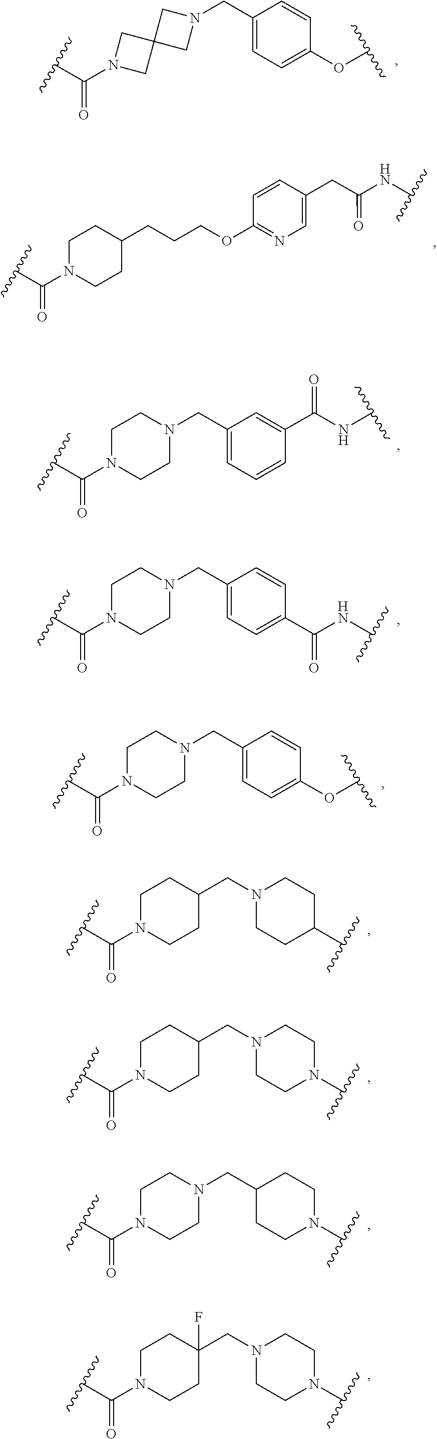

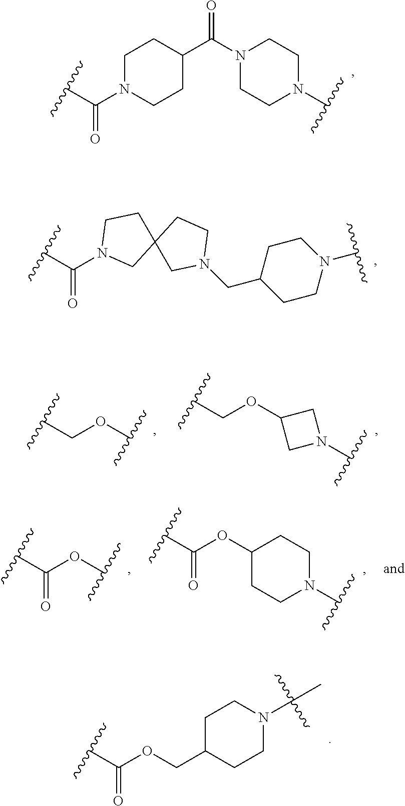

As described herein and exemplified in various compounds, various linkers are of short lengths (e.g., shortest path chain lengths are about or no more than about 8, 9 or 10 atoms) and/or contain few conformationally flexible chain atoms, e.g., they contain about or no more than about 1, 2, 3, 4, or 0, non-ring sp³ C, N, O, or S atoms in its shortest path chain. In some embodiments, there are about or no more than about 1, 2 or 3 non-ring atoms in its shortest path chain. In some embodiments, there are about or no more than about 2 non-ring atoms in its shortest path chain. In some embodiments, there is about or no more than about 1 non-ring atom in its shortest path chain (e.g., as in

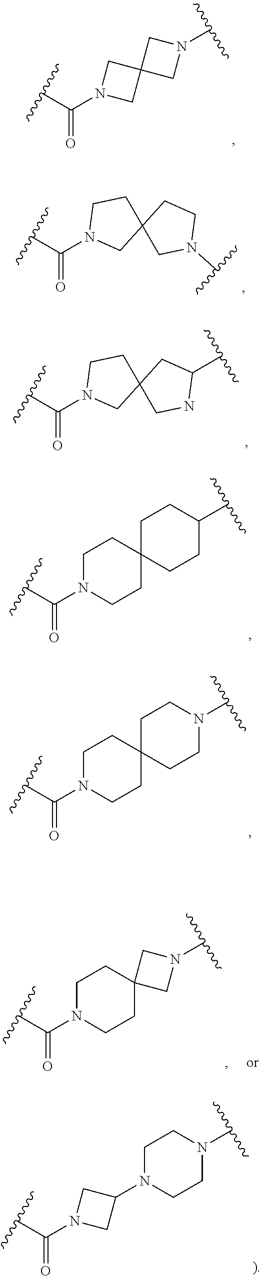

In some embodiments, the number of bonds between two non-ring sp³ atom in the shortest path chain of a linker is about or no more than 1-2, 1-3, 1-4, 1, 2, 3, 4, or 5; in some embodiments, it is 0 (e.g., as in

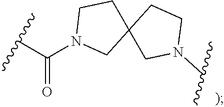 [SMILES: *C(=O)N1CCC2(CCN(*)C2)C1]

in some embodiments, it is 1 (e.g., as in

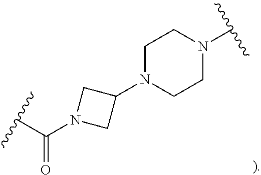 [SMILES: *C(=O)N1CC(N2CCN(*)CC2)C1]

Among other things, such linkers can provide increased conformational rigidity, and/or can provide various benefits and advantages. In some embodiments, rings are monocyclic or bicyclic, in many cases spiro-bicyclic, and each monocyclic ring unit is independently about 3-, 4-, 5- or 6-membered. In some embodiments, rings independently contain 1, 2, or 3 heteroatoms. In some embodiments, one or more heteroatoms are nitrogen. In some embodiments, each heteroatom is nitrogen. In some embodiments, linker comprises one or more basic nitrogen atoms, e.g., in its shortest path chain. For example, in some embodiments, linker is

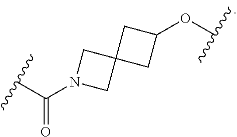 [SMILES: *OC1CC2(C1)CN(C(*)=O)C2]

In some embodiments, linker is

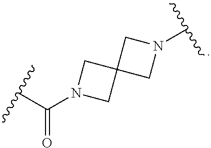 [SMILES: *C(=O)N1CC2(CN(*)C2)C1]

In some embodiments, linker is

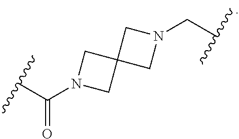 [SMILES: *CN1CC2(C1)CN(C(*)=O)C2]

In some embodiments, linker is

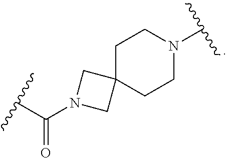 [SMILES: *C(=O)N1CC2(CCN(*)CC2)C1]

In some embodiments, linker is

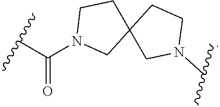 [SMILES: *C(=O)N1CCC2(CCN(*)C2)C1]

In some embodiments, linker is

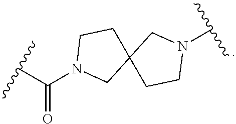 [SMILES: *C(=O)N1CCC2(CCN(*)C2)C1]

In some embodiments, linker is

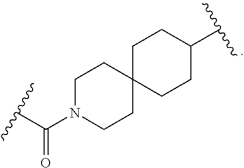 [SMILES: *C(=O)N1CCC2(CCC(*)CC2)CC1]

In some embodiments, linker is

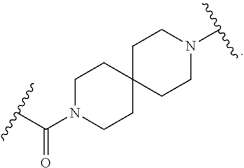 [SMILES: *C(=O)N1CCC2(CCN(*)CC2)CC1]

In some embodiments, linker is

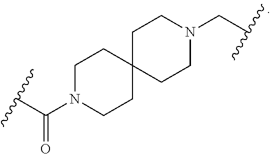 [SMILES: *CN1CCC2(CC1)CCN(C(*)=O)CC2]

In some embodiments, linker is

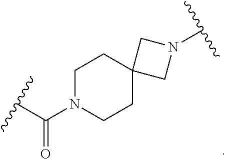 [SMILES: *C(=O)N1CCC2(CC1)CN(*)C2]

In some embodiments, linker is

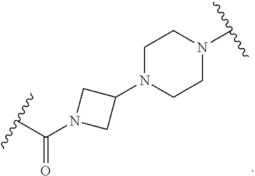 [SMILES: *C(=O)N1CC(N2CCN(*)CC2)C1]

In some embodiments, linker is

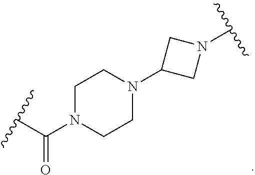 [SMILES: *C(=O)N1CCN(C2CN(*)C2)CC1]

In some embodiments, linker is

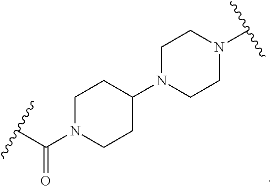 [SMILES: *C(=O)N1CCC(N2CCN(*)CC2)CC1]

In some embodiments, linker is

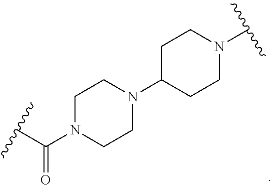 [SMILES: *C(=O)N1CCN(C2CCN(*)CC2)CC1]

In some embodiments, —C(O)— in linker is bonded to PBM. In some embodiments, linker is —CH₂—O—. In some embodiments, linker is

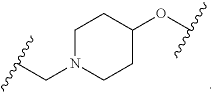 [SMILES: *CN1CCC(O*)CC1]

In some embodiments, linker is bonded to LBM at O.

In some embodiments of any Formulae described herein, linker is not:

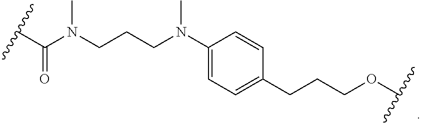 [SMILES: *OCCCc1ccc(N(C)CCCN(C)C(*)=O)cc1]

In some embodiments of any Formulae described herein, each Cy is independently an optionally substituted, mono- or bicyclic, 3- to 11-membered bivalent ring system, wherein the ring system is fully saturated, partially saturated, or aromatic, and the ring system contains 0-4 heteroatoms independently selected from N, O, and S. In some embodiments, each Cy is independently an optionally substituted group selected from phenyl, C₉₋₁₀ bicyclic aryl, 5- to 6-membered monocyclic heteroaryl having 1-4 heteroatoms independently selected from N, O, and S, 8- to 10-membered bicyclic heteroaryl having 1-4 heteroatoms independently selected from N, O, and S, monocyclic C₃₋₇ cycloaliphatic, 5- to 10-membered bicyclic cycloaliphatic, monocyclic 4- to 7-membered heterocyclyl having 1-2 heteroatoms independently selected from N, O, and S, and bicyclic 6- to 11-membered heterocyclyl having 1-3 heteroatoms independently selected from N, O, and S. In some embodiments, each Cy is independently an optionally substituted group selected from phenyl, 5- to 6-membered monocyclic heteroaryl having 1-4 heteroatoms independently selected from N, O, and S, monocyclic 4- to 7-membered heterocyclyl having 1-2 heteroatoms independently selected from N, O, and S, and bicyclic 6- to 11-membered heterocyclyl having 1-3 heteroatoms independently selected from N, O, and S.

In some embodiments, Cy is optionally substituted phenyl. In some embodiments, Cy is phenyl. In some embodiments, Cy is not phenyl.

In some embodiments, Cy is optionally substituted C₉₋₁₀ bicyclic aryl. In some embodiments, Cy is optionally substituted C₁₃₋₁₆ polycyclic aryl.

In some embodiments, Cy is optionally substituted 5- to 6-membered monocyclic heteroaryl having 1-4 heteroatoms independently selected from N, O, and S. In some embodiments, Cy is an optionally substituted 5-membered monocyclic heteroaryl having 1-4 heteroatoms independently selected from N, O, and S. In some embodiments, Cy is an optionally substituted triazole. In some embodiments, Cy is an optionally substituted 6-membered monocyclic heteroaryl having 1-4 heteroatoms independently selected from N, O, and S. In some embodiments, Cy is an optionally substituted pyridine, pyridazine, or pyrimidine.

In some embodiments, Cy is an optionally substituted 8- to 10-membered bicyclic heteroaryl having 1-4 heteroatoms independently selected from N, O, and S. In some embodiments, Cy is an optionally substituted 10- to 16-membered polycyclic heteroaryl having 1-4 heteroatoms independently selected from N, O, and S.

In some embodiments, Cy is an optionally substituted monocyclic C₃₋₇ cycloaliphatic. In some embodiments, Cy is an optionally substituted monocyclic C₃₋₇ cycloalkyl. In some embodiments, Cy is an optionally substituted monocyclic C₄₋₆ cycloalkyl (e.g., cyclobutane, cyclopentane, or cyclohexane).

In some embodiments, Cy is an optionally substituted 5- to 10-membered bicyclic cycloaliphatic. In some embodiments, Cy is an optionally substituted 6- to 10-membered bicyclic cycloaliphatic. In some embodiments, Cy is an optionally substituted bicyclic 6- to 10-membered bridged, fused, or spirocyclic cycloaliphatic.

In some embodiments, Cy is an optionally substituted monocyclic 4- to 7-membered heterocyclyl having 1-2 heteroatoms independently selected from N, O, and S. In some embodiments. Cy is an optionally substituted monocyclic 5- to 6-membered heterocyclyl having 1-2 heteroatoms independently selected from N, O, and S. In some embodiments, Cy is an optionally substituted monocyclic 4-membered heterocyclyl having 1 heteroatom independently selected from N, O, and S. In some embodiments, Cy is azetidine. In some embodiments, Cy is an optionally substituted monocyclic 5-membered heterocyclyl having 1-2 heteroatoms independently selected from N, O, and S. In some embodiments, Cy is pyrrolidine. In some embodiments, Cy is an optionally substituted monocyclic 6-membered heterocyclyl having 1-2 heteroatoms independently selected from N, O, and S. In some embodiments, Cy is piperidine or piperazine. In some embodiments, Cy is an optionally substituted monocyclic 7-membered heterocyclyl having 1-2 heteroatoms independently selected from N, O, and S. In some embodiments, at least one heteroatom is N; in some embodiments, each is N.

In some embodiments, Cy is an optionally substituted bicyclic 6- to 11-membered heterocyclyl having 1-3 heteroatoms independently selected from N, O, and S. In some embodiments, Cy is an optionally substituted bicyclic 6- to 11-membered bridged, fused, or spirocyclic heterocyclyl having 1-3 heteroatoms independently selected from N, O, and S. In some embodiments, Cy is an optionally substituted bicyclic 7- to 11-membered spirocyclic heterocyclyl having 1-3 heteroatoms independently selected from N, O, and S. In some embodiments, Cy is an optionally substituted bicyclic 7-membered spirocyclic heterocyclyl having 1-3 heteroatoms independently selected from N, O, and S. In some embodiments, Cy is an optionally substituted bicyclic 7-membered bridged heterocyclyl having 1-3 heteroatoms independently selected from N, O, and S. In some embodiments, Cy is an optionally substituted bicycle 8-membered spirocyclic heterocyclyl having 1-3 heteroatoms independently selected from N, O, and S. In some embodiments, Cy is an optionally substituted bicyclic 9-membered spirocyclic heterocyclyl having 1-3 heteroatoms independently selected from N, O, and S. In some embodiments, Cy is an optionally substituted bicyclic 10-membered spirocyclic heterocyclyl having 1-3 heteroatoms independently selected from N, O, and S. In some embodiments, Cy is an optionally substituted bicyclic 11-membered spirocyclic heterocyclyl having 1-3 heteroatoms independently selected from N, O, and S. In some embodiments, Cy is an optionally substituted group selected from

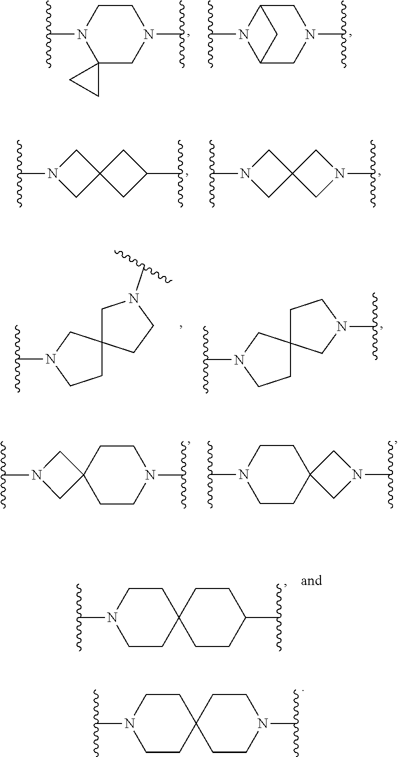

In some embodiments, Cy is selected from:

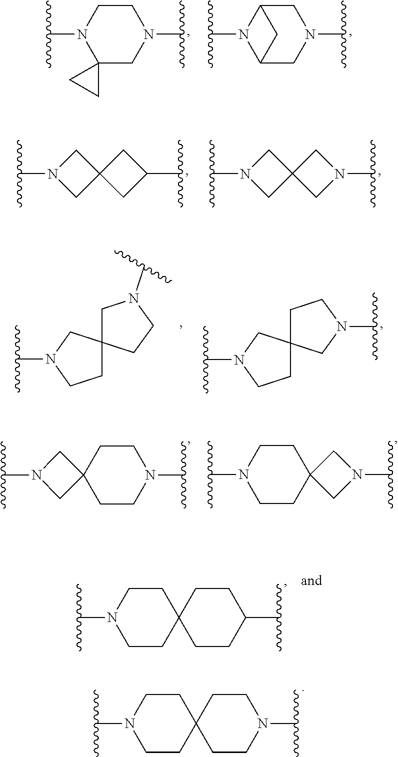

In some embodiments, Cy is an optionally substituted bicyclic 10- to 16-membered heterocyclyl having 1-4 heteroatoms independently selected from N, O, and S. In some embodiments, -Cy- is spiro-bicyclic. In some embodiments, -Cy- is optionally substituted spiro-bicyclic diamine. In some embodiments, -Cy- is optionally substituted spiro-bicyclic diamine boned to the rest of a compound at the two amino groups.

In some embodiments, Cy is an optionally substituted group selected from

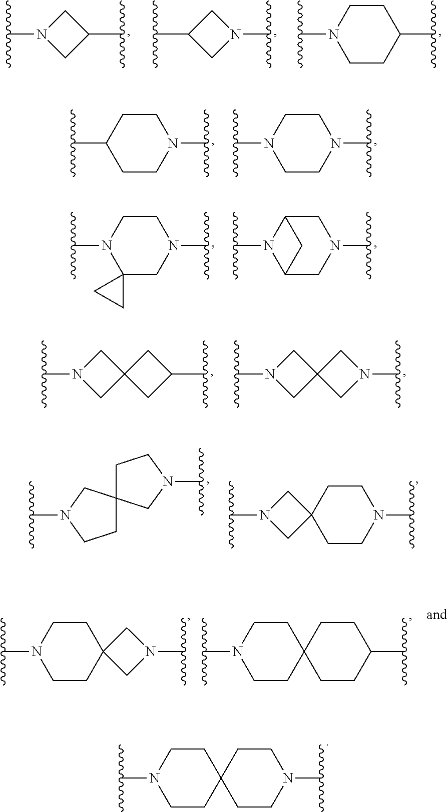

In some embodiments, Cy is selected from:

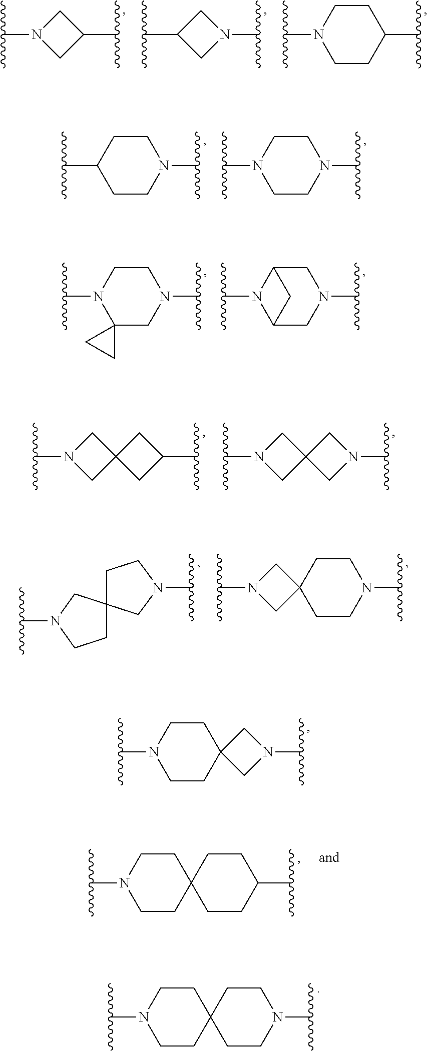

In some embodiments, linker comprises -Cy-. In some embodiments, linker is -Cy-.

Rings are utilized in many formulae and compounds of the present disclosure. In some embodiments, each monocyclic ring unit in a ring (e.g., -Cy-, certain R embodiments, Ring B, Ring C, or Ring F) is independently 3-10 membered (e.g., 3-8, 3-6, 3-5, 4-6, 5-6, 3, 4, 5, 6, 7, 8, 9 or 10), is independently saturated, partially unsaturated or aromatic, and has independently 0-4 (e.g., 0, 1-4, 1-2, 1, 2, 3, or 4) heteroatoms independently selected from N, O, and S. In some embodiments, each monocyclic ring unit is independently 3-8 membered. In some embodiments, each monocyclic ring unit is independently 3-6 membered. In some embodiments, each monocyclic ring unit is independently 5-6 membered. In some embodiments, a monocyclic ring unit is 3-membered. In some embodiments, a monocyclic ring unit is 4-membered. In some embodiments, a monocyclic ring unit is 5-membered. In some embodiments, a monocyclic ring unit is 6-membered. In some embodiments, a monocyclic ring unit is saturated. In some embodiments, each is saturated. In some embodiments, a monocyclic ring unit is partially unsaturated. In some embodiments, a monocyclic ring unit is aromatic. In some embodiments, a monocyclic ring unit is heteroaromatic. In some embodiments, a ring is saturated. In some embodiments, a ring is partially unsaturated. In some embodiments, a ring is aromatic. In some embodiments, a ring is heteroaromatic. In some embodiments, a monocyclic ring unit has no heteroatom. In some embodiments, a monocyclic ring unit has 1-4 (e.g., 1-3, 1-2, 1, 2, 3, or 4) heteroatoms independently selected from N, O, and S. In some embodiments, each monocyclic ring unit independently has 1-4 (e.g., 1-3, 1-2, 1, 2, 3, or 4) heteroatoms independently selected from N, O, and S. In some embodiments, a monocyclic ring unit has a nitrogen atom. In some embodiments, each monocyclic ring unit independently has a nitrogen atom.

Various variables can be R′ as described herein. In some embodiments, R′ is hydrogen.

In some embodiments, R′ is —R as described herein. In some embodiments, R′ is —C(O)R wherein R is as described herein. In some embodiments, R′ is —C(O)OR wherein R is as described herein. In some embodiments, R′ is —S(O)₂R wherein R is as described herein. In some embodiments, two R′ groups, or two groups that are or can be R′, on the same atom can be taken together with the atom to form an optionally substituted ring as described herein. In some embodiments, two R′ attached to the same atom, together with the atom to which they are attached, combine to form an optionally substituted 3-16 (e.g., 3, 4, 5, 6, 7, 8, 9, 10, 11, 12, 13, 14, 15, 16, 3-15, 3-10, 5-10, 3-8, 3-7 or 5-6) membered ring having 1-5 (e.g., 1-2, 1-3, 1-4, 1, 2, 3, 4, or 5) heteroatoms independently selected from N, O, and S. In some embodiments, two R′ groups on the same atom are optionally and independently taken together with the atom to form an optionally substituted 3-10 (e.g., 3, 4, 5, 6, 7, 8, 9, 10, 11, 12, 13, 14, 15, 16, 5-10, 3-8, 3-7 or 5-6) membered ring having, in addition to the atom, 0-4 (e.g., 0), 1, 2, 3, or 4) heteroatoms. In some embodiments, a formed ring is substituted (in addition to groups attached to the intervening atom(s). In some embodiments, a formed ring is unsubstituted. In some embodiments, a formed ring is 3-membered. In some embodiments, a formed ring is 4-membered. In some embodiments, a formed ring is 5-membered. In some embodiments, a formed ring is 6-membered. In some embodiments, a formed ring is 7-membered. In some embodiments, a formed ring is 8-membered. In some embodiments, a formed ring is 9-membered. In some embodiments, a formed ring is 10-membered. In some embodiments, a formed ring is saturated. In some embodiments, a formed ring is partially unsaturated. In some embodiments, a formed ring is aromatic. In some embodiments, a formed ring is monocyclic. In some embodiments, it is bicyclic. In some embodiments, it is polycyclic. In some embodiments, each monocyclic unit is independently a 3-10 (e.g., 3-8, 3-6, 5-6, or 3, 4, 5, 6, 7, 8, 9, or 10) membered ring which is independently saturated, partially unsaturated or aromatic and has 0-4 (e.g., 0, 1, 2, 3, or 4) heteroatoms. In some embodiments, each monocyclic unit is independently a 3-10 (e.g., 3-10, 3-8, 3-6, 5-6, or 3, 4, 5, 6, 7, 8, 9, or 10) membered ring which is independently saturated, partially unsaturated or aromatic and has 0-4 (e.g., 0, 1, 2, 3, or 4) heteroatoms independently selected from nitrogen, oxygen and sulfur. In some embodiments, each monocyclic ring unit is independently 3-7 membered. In some embodiments, each monocyclic ring unit is independently 3-6 membered. In some embodiments, each monocyclic ring unit is independently 5-7 membered. In some embodiments, each monocyclic unit is independently saturated or partially unsaturated. In some embodiments, at least one monocyclic unit is saturated. In some embodiments, at least one monocyclic unit is partially unsaturated. In some embodiments, at least one monocyclic unit is aromatic. In some embodiments, a formed ring has, in addition to the intervening atom(s), 0-4 (e.g., 0, 1, 2, 3, or 4) heteroatoms independently selected from oxygen, nitrogen, sulfur, phosphorus and silicon. In some embodiments, there are no additional heteroatoms. In some embodiments, there is one additional heteroatom. In some embodiments, there are 2 additional heteroatoms. In some embodiments, there are 3 additional heteroatoms. In some embodiments, there are 4 additional heteroatoms. In some embodiments, there are 5 additional heteroatoms. In some embodiments, there are 6 or more additional heteroatoms. In some embodiments, an additional heteroatom is nitrogen. In some embodiments, an additional heteroatom is oxygen. In some embodiments, an additional heteroatom is sulfur.

Various variables can be R as described herein. Various embodiments for R are extensively described herein, including in various sections for other variables that can be R (e.g., R¹, R², or R′).

In some embodiments, R is —H. In some embodiments, R is not —H.

In some embodiments, R is optionally substituted C₁₋₈ (e.g., C₁₋₇, C₁₋₆, C₁₋₅, C₁₋₄, C₁, C₂, C₃, C₄, C₅, C₆, C₇, or C₈) aliphatic. In some embodiments, R is optionally substituted C₁₋₆ aliphatic. In some embodiments, R is optionally substituted C₁₋₆ alkyl. In some embodiments, R is optionally substituted methyl. In some embodiments, R is optionally substituted ethyl. In some embodiments, R is optionally substituted n-propyl. In some embodiments. R is optionally substituted isopropyl. In some embodiments, R is n-butyl. In some embodiments, R is t-butyl. In some embodiments, R is pentyl. In some embodiments, R is hexyl.

In some embodiments, R is optionally substituted C₁₋₈ (e.g., C₁₋₇, C₁₋₆, C₁₋₅, C₁₋₄, C₁, C₂, C₃, C₄, C₅, C₆, C₇, or C₈) heteroaliphatic having 1-3 (e.g., 1, 2, or 3) heteroatoms independently selected from N, O, and S. In some embodiments, R is optionally substituted C₁₋₆ heteroaliphatic having 1-3 (e.g., 1, 2, or 3) heteroatoms independently selected from N, O, and S. In some embodiments, a heteroatom is nitrogen. In some embodiments, a heteroatom is oxygen. In some embodiments, a heteroatom is sulfur.

In some embodiments, R is optionally substituted C₃₋₁₀ (e.g., C₄₋₁₀, C₃₋₉, C₃₋₇, or 3, 4, 5, 6, 7, 8, 9, or 10-membered) cycloaliphatic. In some embodiments, a cycloaliphatic group is a cycloalkyl group. In some embodiments, a cycloaliphatic group is monocyclic. In some embodiments, it is bicyclic. In some embodiments, it is polycyclic. In some embodiments, each monocyclic unit is independently a 3-10 (e.g., C₄₋₁₀, C₃₋₉, C₃₋₇, or 3, 4, 5, 6, 7, 8, 9, or 10) membered cycloaliphatic ring. In some embodiments, a cycloaliphatic group is saturated. In some embodiments, it is partially unsaturated. In some embodiments, R is optionally substituted cyclopropyl. In some embodiments, R is optionally substituted cyclobutyl. In some embodiments, R is optionally substituted cyclopentyl. In some embodiments, R is optionally substituted cyclohexyl. In some embodiments, R is optionally substituted cycloheptyl. In some embodiments, R is cyclopropyl. In some embodiments, R is cyclobutyl. In some embodiments, R is cyclopentyl. In some embodiments, R is cyclohexyl. In some embodiments, R is cycloheptyl.

In some embodiments, R is optionally substituted 3-10 (e.g., 3-9, 3-6, 3-5, or 3, 4, 5, 6, 7, 8, 9, or 10) membered heterocyclyl having 1-5 (e.g., 1-2, 1-3, 1-4, 1, 2, 3, 4, or 5) heteroatoms independently selected from N, O, and S. In some embodiments, R is optionally substituted 3-10 (e.g., 3-9, 3-6, 3-5, or 3, 4, 5, 6, 7, 8, 9, or 10) membered heterocyclyl having 1-4 (e.g., 1-2, 1, 2, 3, or 4) heteroatoms independently selected from N, O, and S. In some embodiments, a heterocyclyl group is monocyclic. In some embodiments, it is bicyclic. In some embodiments, it is polycyclic. In some embodiments, each monocyclic unit is independently a 3-10 (e.g., 3-8, 3-6, 3-5, 4-6, 5-6, 3, 4, 5, 6, 7, 8, 9 or 10) membered heterocyclyl ring having 1-4 (e.g., 1, 2, 3, or 4) heteroatoms independently selected from N, O, and S. In some embodiments, a heterocyclyl group is saturated. In some embodiments, it is partially unsaturated. In some embodiments, a heterocyclyl ring has one heteroatom. In some embodiments, a heterocyclyl ring has two or more heteroatoms. In some embodiments, a heterocyclyl ring has three or more heteroatoms. In some embodiments, a heterocyclyl ring has four or more heteroatoms. In some embodiments, a heteroatom is nitrogen. In some embodiments, a heteroatom is oxygen. In some embodiments, a heteroatom is sulfur.

In some embodiments, R is optionally substituted C₆₋₁₀ (e.g., C₆ or C₁₀) aryl. In some embodiments. R is optionally substituted C₆ aryl. In some embodiments, R is optionally substituted C₁₀ aryl. In some embodiments, an aryl ring is monocyclic. In some embodiments, an aryl ring is bicyclic. In some embodiments, an aryl ring is polycyclic. In some embodiments, each monocyclic unit is independently a 6-membered aromatic ring. In some embodiments, R is optionally substituted phenyl. In some embodiments, R is phenyl. In some embodiments, R is optionally substituted 10-membered aryl. In some embodiments, R is optionally substituted naphthyl. In some embodiments, R is naphthyl.

In some embodiments, R is optionally substituted 5-10 (e.g., 5-9, 5-6, 5, 6, 7, 8, 9, or 10) membered heteroaryl having 1-5 (e.g., 1-5, 1-4, 1, 2, 3, 4, or 5) heteroatoms independently selected from N, O, and S. In some embodiments, R is 5-10 (e.g., 5-9, or 5, 6, 9, or 10) membered heteroaryl having 1-4 (e.g., 1, 2, 3, or 4) heteroatoms independently selected from N, O, and S. In some embodiments, a heteroaryl ring is monocyclic. In some embodiments, a heteroaryl ring is bicyclic. In some embodiments, a heteroaryl ring is polycyclic. In some embodiments, each monocyclic unit is independently a 5- or 6-membered aromatic ring having 0-4 heteroatoms, e.g., independently selected from N, O, and S, wherein at least one monocyclic unit contains 1-4 heteroatoms. In some embodiments. R is optionally substituted 5-membered monocyclic heteroaryl having 1-4 heteroatoms independently selected from N, O, and S. In some embodiments, R is optionally substituted 6-membered monocyclic heteroaryl having 1-4 heteroatoms independently selected from N, O, and S. In some embodiments, R is optionally substituted 9-membered bicyclic heteroaryl having 1-4 heteroatoms independently selected from N, O, and S. In some embodiments, R is optionally substituted 10-membered bicyclic heteroaryl having 1-4 heteroatoms independently selected from N, O, and S. In some embodiments, a heteroaryl ring has one heteroatom. In some embodiments, a heteroaryl ring has two or more heteroatoms. In some embodiments, a heteroaryl ring has three or more heteroatoms. In some embodiments, a heteroaryl ring has four or more heteroatoms. In some embodiments, a heteroatom is nitrogen. In some embodiments, a heteroatom is oxygen. In some embodiments, a heteroatom is sulfur.

In some embodiments of any Formulae described herein, each R is independently hydrogen, optionally substituted C₁₋₆ aliphatic, or optionally substituted C₃₋₇ cycloaliphatic. In some embodiments, each R is independently hydrogen or optionally substituted C₁₋₆ aliphatic. In some embodiments, each R is independently hydrogen or optionally substituted C₁₋₆ alkyl. In some embodiments, R is hydrogen. In some embodiments, R is optionally substituted C₁₋₆ aliphatic. In some embodiments, R is optionally substituted C₁₋₆ alkyl. In some embodiments, R is C₁₋₆ alkyl. In some embodiments, R is optionally substituted C₁₋₂ alkyl. In some embodiments, R is C₁₋₂ alkyl (e.g., methyl). In some embodiments, each R is C₁₋₂ alkyl (e.g., methyl). In some embodiments, R is optionally substituted phenyl. In some embodiments, R is optionally substituted C₃₋₇ cycloaliphatic. In some embodiments, R is optionally substituted C₃₋₇ cycloalkyl (e.g., cyclopropyl). In some embodiments, R is optionally substituted 5- to 6-membered monocyclic heteroaryl having 1-4 heteroatoms independently selected from N, O, and S. In some embodiments, R is optionally substituted 5-membered monocyclic heteroaryl having 1-4 heteroatoms independently selected from N, O, and S. In some embodiments. R is optionally substituted 6-membered monocyclic heteroaryl having 1-4 heteroatoms independently selected from N, O, and S. In some embodiments, R is optionally substituted 3- to 7-membered monocyclic heterocyclyl having 1-2 heteroatoms independently selected from N, O, and S. In some embodiments, R is optionally substituted 4- to 6-membered monocyclic heterocyclyl having 1-2 heteroatoms independently selected from N, O, and S.

As described herein, various groups may be optionally substituted. Substituents are routinely utilized in chemistry including in development of various therapeutics. Many substituents can be utilized in accordance with the present disclosure. In some embodiments, an optionally substituted group is unsubstituted. In some embodiments, an optionally substituted group is substituted. In some embodiments, substituents are those that result in the formation of compounds for a desired property, activity, use, etc., as described herein. In some embodiments, compounds are stable for therapeutic use as described herein. The term “stable,” as used herein, refers to compounds that are not substantially altered when subjected to conditions to allow for their production, detection, and, in certain embodiments, their recovery, purification, and use for one or more of the purposes disclosed herein. In some embodiments, a substituent is a hydrocarbon group. In some embodiments, a substituent comprises a heteroatom. In some embodiments, a substituent comprises multiple heteroatoms. In some embodiments, each atom in a substituent is independently selected from hydrogen, carbon, halogen, nitrogen, oxygen, sulfur, phosphorus and silicon. In some embodiments, each atom in a substituent is independently selected from hydrogen, carbon, halogen, nitrogen, oxygen, and sulfur. In some embodiments, each atom in a substituent is independently selected from hydrogen, carbon, fluorine, chlorine, bromine, iodine, nitrogen, oxygen, and sulfur. In some embodiments, the total number of carbon and non-halogen heteroatom(s) in a substituent is about or no more than about 1; in some embodiments, it is about or no more than about 2; in some embodiments, it is about or no more than about 3; in some embodiments, it is about or no more than about 4; in some embodiments, it is about or no more than about 5; in some embodiments, it is about or no more than about 6; in some embodiments, it is about or no more than about 7; in some embodiments, it is about or no more than about 8; in some embodiments, it is about or no more than about 9; in some embodiments, it is about or no more than about 10; in some embodiments, it is about or no more than about 11; in some embodiments, it is about or no more than about 12; in some embodiments, it is about or no more than about 13; in some embodiments, it is about or no more than about 14; in some embodiments, it is about or no more than about 15; in some embodiments, it is about or no more than about 20. In some embodiments, the total number of carbon and non-halogen heteroatom(s) in each substituent is independently no more than about 1, 2, 3, 4, 5, 6, 7, 8, 9, 10, 11, 12, 13, 14, 15, 16, 17, 18, 19, or 20. In some embodiments, the total number of carbon and non-halogen heteroatom(s) in each substituent is independently no more than about 15. In some embodiments, the total number of carbon and non-halogen heteroatom(s) in each substituent is independently no more than about 10. In some embodiments, the total number of carbon and non-halogen heteroatom(s) in each substituent is independently no more than about 6. Substituents of various sizes are described herein including exemplified in various compounds. In some embodiments, each optional substituent on a substitutable group (e.g., Ring A, Ring B, R, etc.) is independently halogen, C₁₋₄ alkyl, —OH, —CN, —NO₂, C₁₋₄ haloalkyl (e.g., —CF₃), —OR^{SB}, —N(R^{SB})₂, —C(O)OR^{SB}, —C(O)N(R^{SB})₂, or —S(O)₂N(R^{SB})₂ wherein each R^{SB} is independently —H, C₁₋₄ alkyl or C₁₋₄ haloalkyl. In some embodiments, each optional substituent on a substitutable group (e.g., Ring A, Ring B, R, etc.) is independently halogen, C₁₋₄ alkyl, C₁₋₄ haloalkyl, or —OH. In some embodiments, each optional substituent on a substitutable group (e.g., Ring A, Ring B, R, etc.) is independently halogen or C₁₋₄ alkyl. As examples, many substituents are exemplified in compounds described herein, e.g., in Table 1.

In some embodiments of any Formulae described herein, linker is attached to the bracketed moiety at one of R^{d}, R^{e}, R^{f}, or R^{g}, or linker is attached to the bracketed moiety at one Rⁱ or the ring formed when two Rⁱ groups are taken together. In some embodiments, linker is attached to the bracketed moiety at one of R^{d}, R^{e}, R^{f}, or R^{g}. In some embodiments, linker is attached to the bracketed moiety at one Rⁱ or the ring formed when two Rⁱ groups are taken together. In some embodiments, linker is attached to a position having —H or a monovalent substituent, e.g., halogen, alkyl, etc. In some embodiments, linker is attached to a position having —H (e.g., for a compound of the structure of PBM-H or LBM-H, linker can replace-H to form a structure of PBM-linker or LBM-linker, respectively).

### LBM

In some embodiments, LBM is

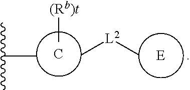

In some embodiments, LBM is

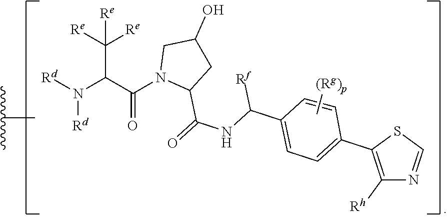

In some embodiments. LBM is

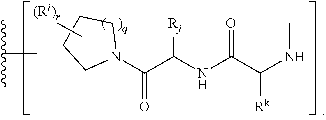

In some embodiments, LBM is

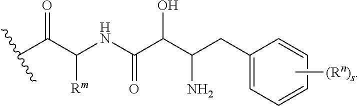

In some embodiments, LBM is

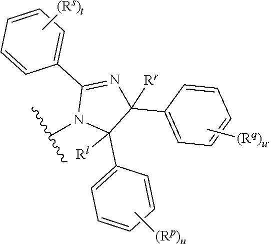

In some embodiments, LBM is

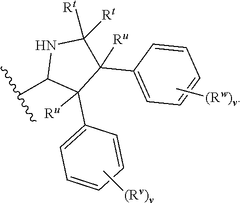

In some embodiments of any Formulae described herein, LBM is an E3 ubiquitin ligase binding moiety, i.e., is a moiety that is capable of binding an E3 ubiquitin ligase. Typically, an LBM is considered to be capable of binding an E3 ubiquitin ligase if it specifically (i.e., preferentially) associates with the E3 ubiquitin ligase when contacted with the E3 ubiquitin ligase in the presence of at least one other protein. In some embodiments, an LBM is considered to be capable of binding an E3 ubiquitin ligase if it specifically associates with that protein within a cell (e.g., in vitro or in vivo). In some embodiments, an LBM is considered to be capable of binding an E3 ubiquitin ligase if a compound of LBM-H or a salt thereof, or LBM-linker-H or a salt thereof, has a K_{D} toward an E3 ubiquitin ligase of about or less than about 1 uM in an assay such as surface plasmon resonance (SPR) or isothermal titration calorimetry (ITC). In some embodiments, a compound of LBM-H or a salt thereof, or LBM-linker-H or a salt thereof, has an IC₅₀ of about or less than about 1 uM in a competition or functional assay, such as time-resolved fluorescence resonance energy transfer (TR-FRET). An LBM may be capable of binding any suitable E3 ubiquitin ligase, including cereblon, Von Hippel-Lindau protein, Inhibitor of Apoptosis protein, MDM2, RNF114, DCAF16, DCAF15, KEAP1, FEM1B, Arylhydrocarbon Receptor, etc. Some moieties capable of binding an E3 ubiquitin ligase are known in the art—see, e.g., Sun, X., et al., Signal Transduction and Targeted Therapy, 2019, 4, 64; Ishida, T., et al., SLAS Discovery, 2021, 26(4), 484-502; Bricelj, A., et al., Frontiers in Chemistry, 2021, 9, Article 707317; Min, J., et al., Angew. Chem. Int. Ed., 2021, 60, 26663-70; and WO 2019/140387, the entire contents of each of which are hereby incorporated by reference. In some embodiments, technologies for identifying or assessing LBM are described in one or more of such references. Certain useful LBM and technologies are described in WO 2017/197036, WO 2017/197046, WO 2017/197051, WO 2017/197055, WO 2017/197056, WO 2018/220149, WO 2018/237026, WO 2019/023553, WO 2019/043208, WO 2019/043214, WO 2019/043217, WO 2019/060693, WO 2019/060742, WO 2019/099868, WO 2019/121562, WO 2019/133531, WO 2019/140380, WO 2019/140387, WO 2019/149922, WO 2019/191112, WO 2019/204354, WO 2019/236483, WO 2020/010177, WO 2020/010210, WO 2020/010227, WO 2020/051235, WO 2020/113233, WO 2020/132561, WO 2020/181232, WO 2020/206424, WO 2020/210630, WO 2020/251969, WO 2020/251971, WO 2020/251972, WO 2020/251974, WO 2020/264490, WO 2020/264499, WO 2021/011631, WO 2021/011634, WO 2021/011868, WO 2021/011871. WO 2021/083949, WO 2021/086785, WO 2021/119159, WO 2021/127190, WO 2021/127278, WO 2021/127283, WO 2021/127561, WO 2021/133917, WO 2021/133920, WO 2021/158634, WO 2021/178920, WO 2021/188696, WO 2021/188948, WO 2021/222366, WO 2021/231778, WO 2021/247897, WO 2021/247899, WO 2021/255212, WO 2021/255213, WO 2021/257914, WO 2022/027058, WO 2022/032026, WO 2022/032132, WO 2022/077010, WO 2022/081925, WO 2022/081927, WO 2022/081928, WO 2022/087216, WO 2022/125790, WO 2022/125800, WO 2022/125804, WO 2022/147465, WO 2022/174268, WO 2022/174269, WO 2022/178532, WO 2022/235945, WO 2022/236058, WO 2022/236339, WO 2022/251539, WO 2022/261250, WO 2023/039208, WO 2023/044046, WO 2023/049790, WO 2023/055952, WO 2023/076161, WO 2023/076556, WO 2023/107706, WO 2023/114933, WO 2023/137439, WO 2023/147594, WO 2023/154417, WO 2023/192578, WO 2023/192586, WO 2023/220425, WO 2023/239645, WO 2023/239697. WO 2023/239750, WO 2023/244764, WO 2023/250058, WO 2023/278402, WO 2023/278759, WO 2023/283372, WO 2023/283610, WO 2024/030628, WO 2024/039901, or WO 2024/039903.

In some embodiments, LBM is

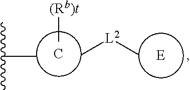

wherein the variables are as defined and described in classes and subclasses herein, both singly and in combination. In some embodiments, certain LBM types and species can provide improved properties and/or activities, e.g., improved stability, improved selectivity (e.g., for KAT2A over KAT2B), improved bioavailability, improved potency, and/or improved half-life. In some embodiments, LBM has the following structure of

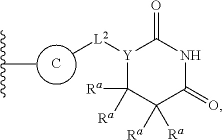

wherein the variables are as defined and described in classes and subclasses herein, both singly and in combination. In some embodiments, LBM has the following structure of

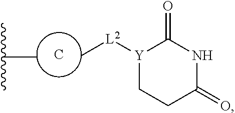

wherein the variables are as defined and described in classes and subclasses herein, both singly and in combination. In some embodiments, LBM has the following structure of

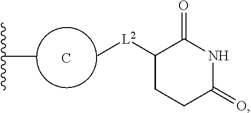

wherein the variables are as defined and described in classes and subclasses herein, both singly and in combination. In some embodiments, LBM has the following structure of

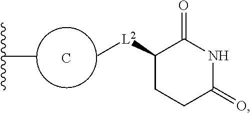

wherein the variables are as defined and described in classes and subclasses herein, both singly and in combination. In some embodiments, LBM has the following structure of

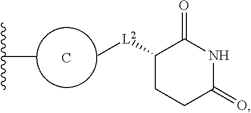

wherein the variables are as defined and described in classes and subclasses herein, both singly and in combination. In some embodiments, LBM has the following structure of

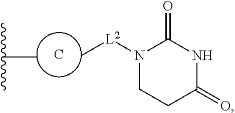

wherein the variables are as defined and described in classes and subclasses herein, both singly and in combination. In some embodiments, Ring C is an optionally substituted phenyl ring. In some embodiments, Ring C is an optionally substituted 5-membered heteroaryl ring having 1-4 heteroatoms independently selected from N, O, and S. In some embodiments, Ring C is an optionally substituted 6-membered heteroaryl ring having 1-4 heteroatoms independently selected from N, O, and S. In some embodiments, Ring C is an optionally substituted naphthyl ring having 1-4 heteroatoms independently selected from N, O, and S. In some embodiments, Ring C is an optionally substituted bicyclic 9-membered heteroary ring having 1-4 heteroatoms independently selected from N, O, and S. In some embodiments, Ring C is an optionally substituted bicyclic 10-membered heteroaryl ring having 1-4 heteroatoms independently selected from N, O, and S. In some embodiments, L² is a covalent bond. In some embodiments, L² is —NH—. In some embodiments, L² is —O—. In some embodiments, L¹ is —C(O)—NH— wherein —C(O)— is bonded to Ring C. In some embodiments, L² is —C(O)—NH— wherein —NH— is bonded to Ring C. In some embodiments, L² is bonded to an atom next to the atom to which linker is bonded. In some embodiments, in the shortest path from the atom to with L² is bonded to the atom to which linker is bonded, there is one atom between the atom to with L² is bonded and the atom to which linker is bonded. In some embodiments, in the shortest path from the atom to with L² is bonded to the atom to which linker is bonded, there are two atoms between the atom to with L² is bonded and the atom to which linker is bonded. In some embodiments, in the shortest path from the atom to with L² is bonded to the atom to which linker is bonded, there are three atoms between the atom to with L² is bonded and the atom to which linker is bonded. In some embodiments, in the shortest path from the atom to with L² is bonded to the atom to which linker is bonded, there four two atoms between the atom to with L² is bonded and the atom to which linker is bonded. For example, in some embodiments, LBM is optionally substituted

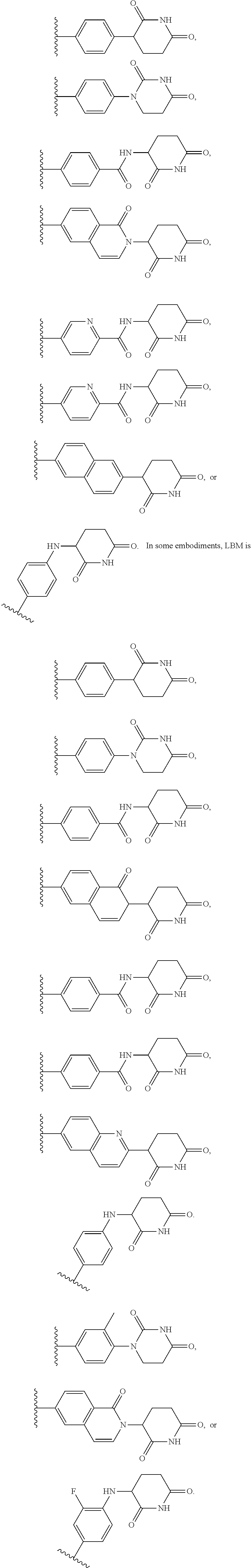

In some embodiments of any Formulae described herein, a LBM is a cereblon (CRBN) binding moiety, i.e., is a moiety that is capable of binding cereblon.

In some embodiments of any Formulae described herein, a LBM has the following structure:

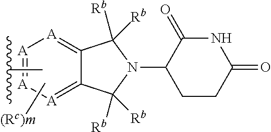

wherein R^{b}, R^{c}, and m are as defined herein for Formula IIIA and described in classes and subclasses herein, both singly and in combination; and each A is independently N, C, or CH, provided that no more than two A groups are N. It will be appreciated that A is C when it is the point of attachment to the rest of the molecule.

In some embodiments of any Formulae described herein, a LBM has the following structure:

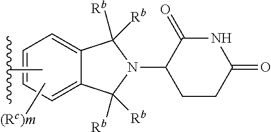

wherein R^{b}, R^{c}, and m are as defined herein for Formula IIIA and described in classes and subclasses herein, both singly and in combination.

In some embodiments of any Formulae described herein, a LBM is selected from:

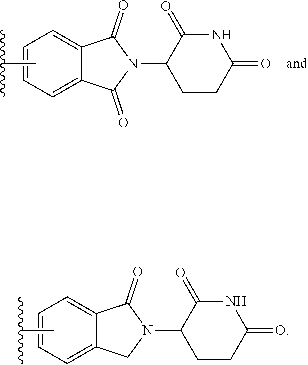

In some embodiments of any Formulae described herein, a LBM has the following structure:

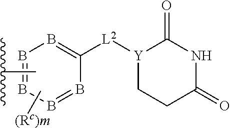

wherein B, L², R^{c}, Y, and m are as defined herein for Formula IIIC and described in classes and subclasses herein, both singly and in combination. It will be appreciated that B is C when it is the point of attachment to the rest of the molecule.

In some embodiments of any Formulae described herein, a LBM has the following structure:

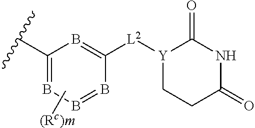

wherein B, L², R^{c}, Y, and m are as defined herein for Formula IIIC and described in classes and subclasses herein, both singly and in combination.

In some embodiments of any Formulae described herein, a LBM has the following structure:

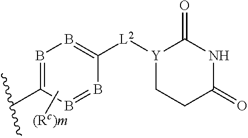

wherein B, L², R^{c}, Y, and m are as defined herein for Formula IIIC and described in classes and subclasses herein, both singly and in combination.

In some embodiments of any Formulae described herein, a LBM is selected from:

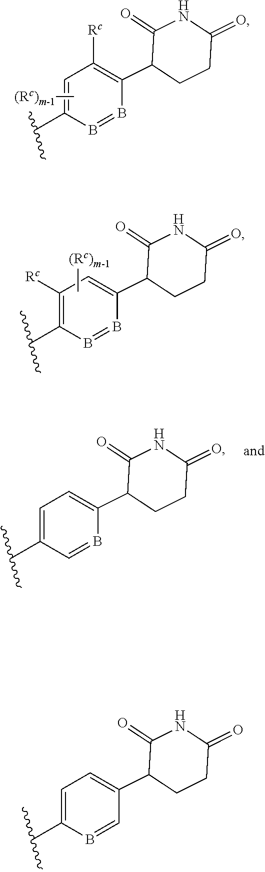

wherein B, L², R^{c}, and m are as defined herein for Formula IIIC and described in classes and subclasses herein, both singly and in combination.

In some embodiments of any Formulae described herein, a LBM is selected from:

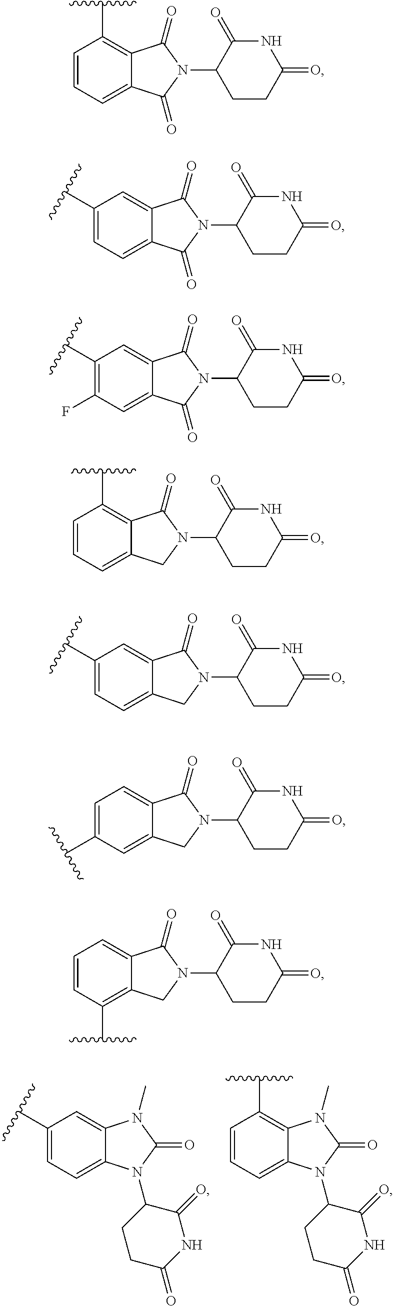

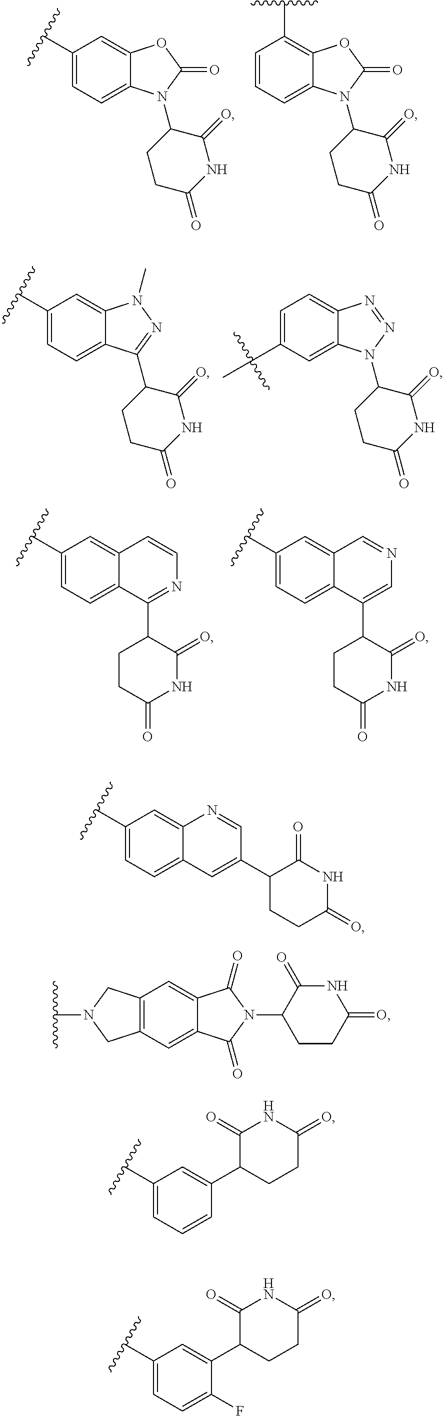

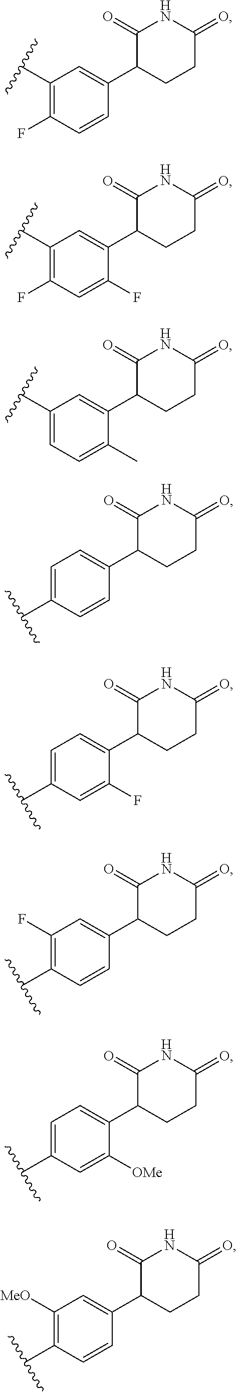

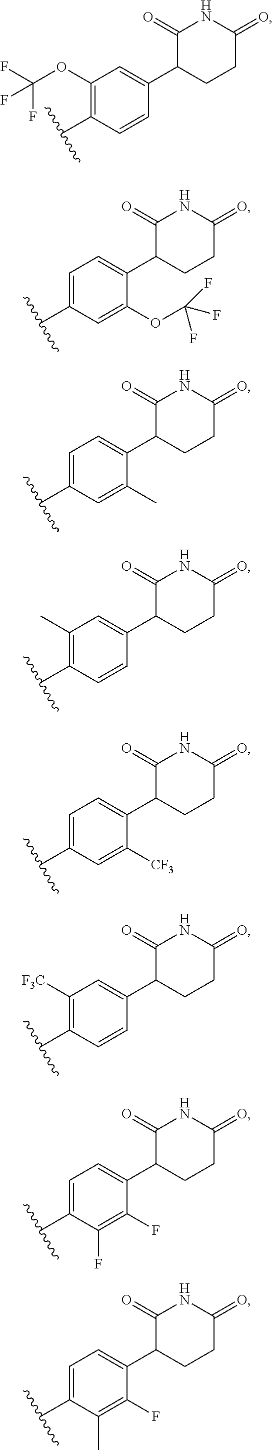

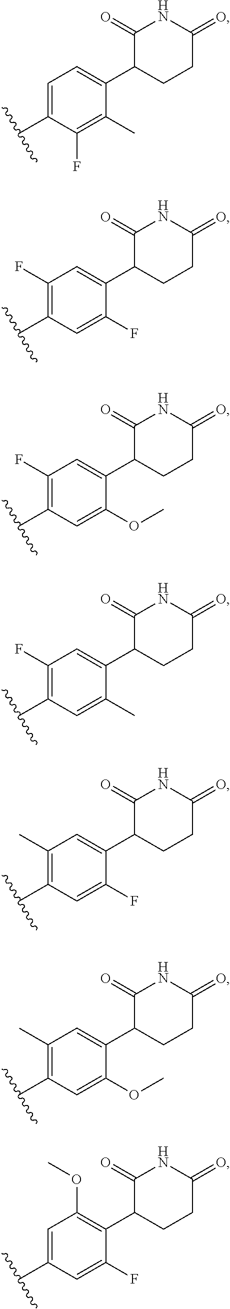

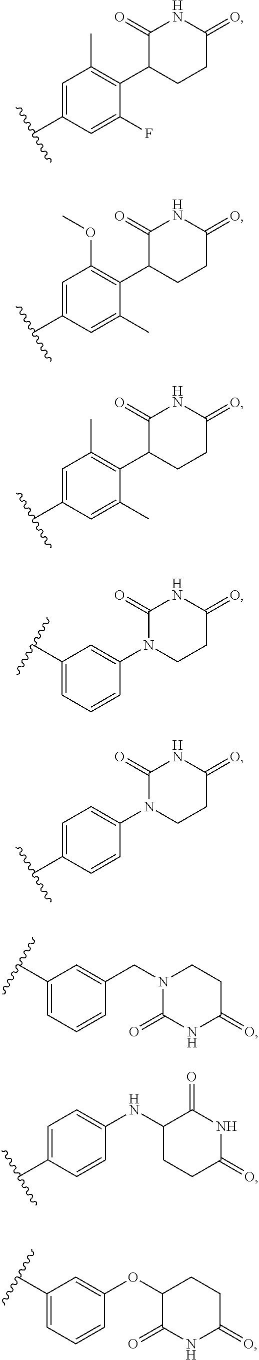

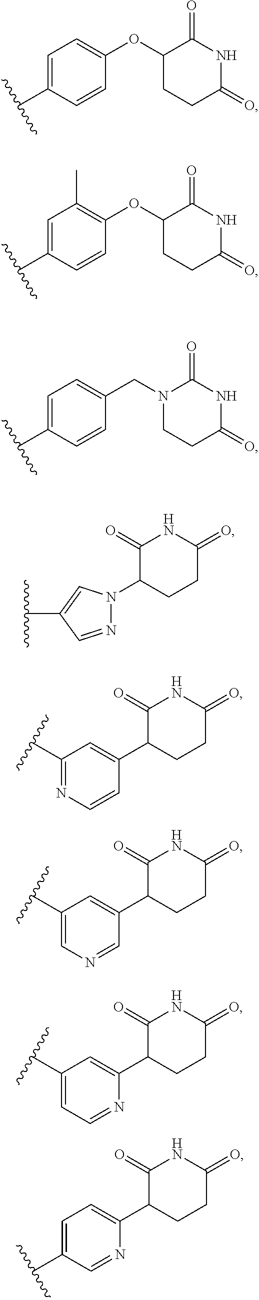

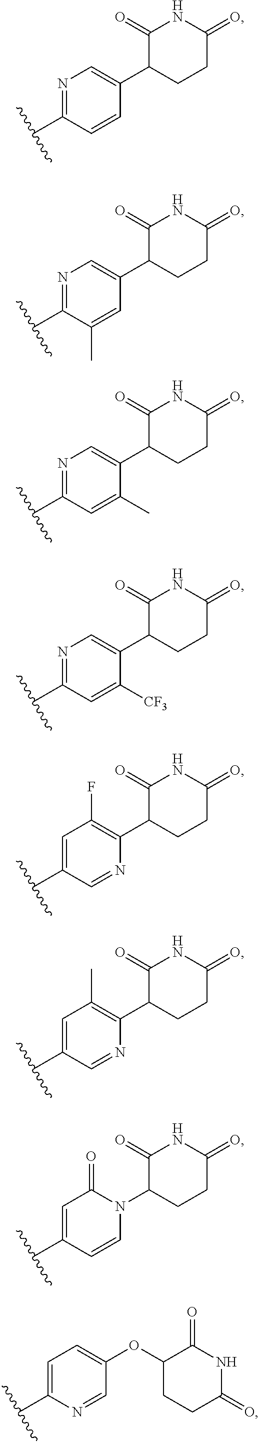

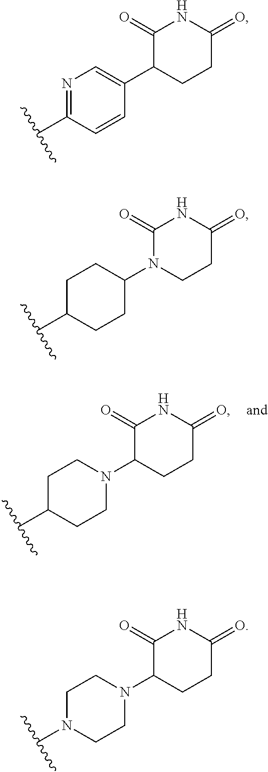 [SMILES: *c1ccc(C2CCC(=O)NC2=O)cn1 ; *C1CCC(N2CCC(=O)NC2=O)CC1 ; *C1CCN(C2CCC(=O)NC2=O)CC1 ; *N1CCN(C2CCC(=O)NC2=O)CC1]

In some embodiments of any Formulae described herein, a LBM is selected from:

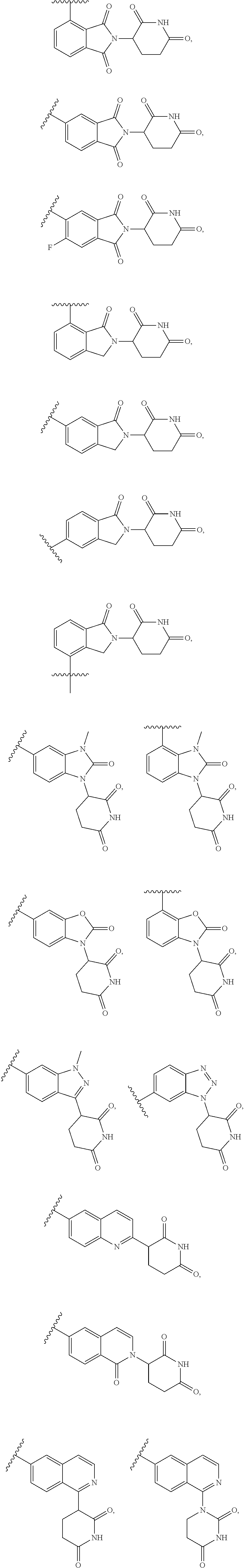

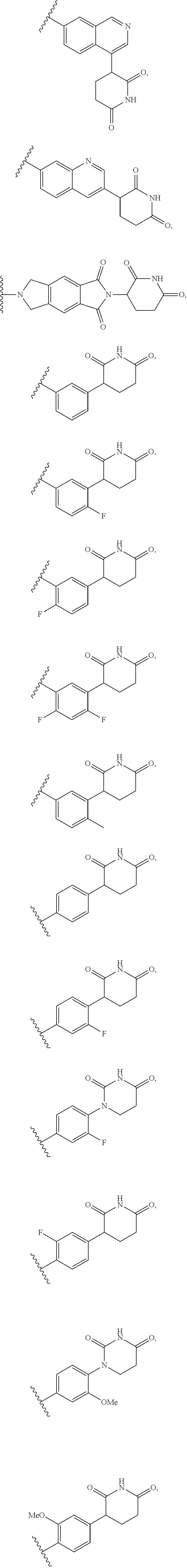

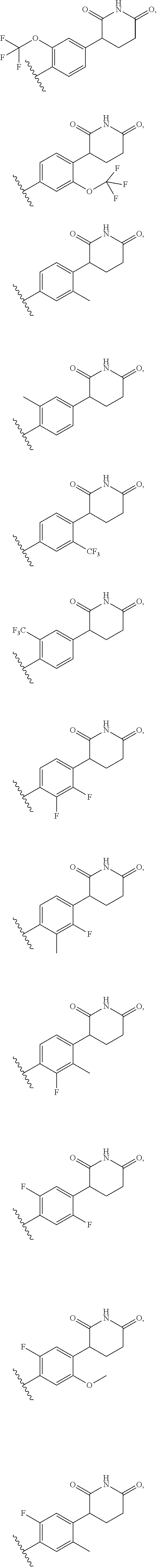

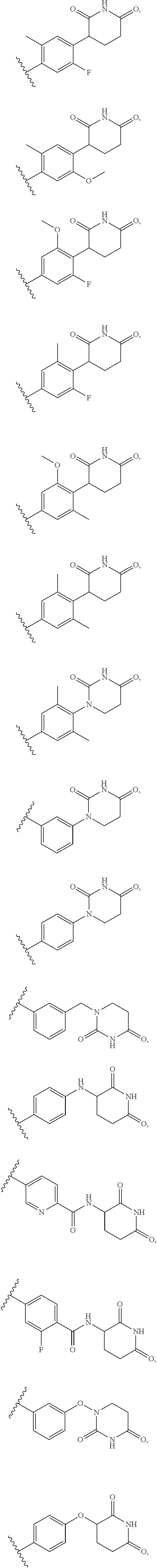

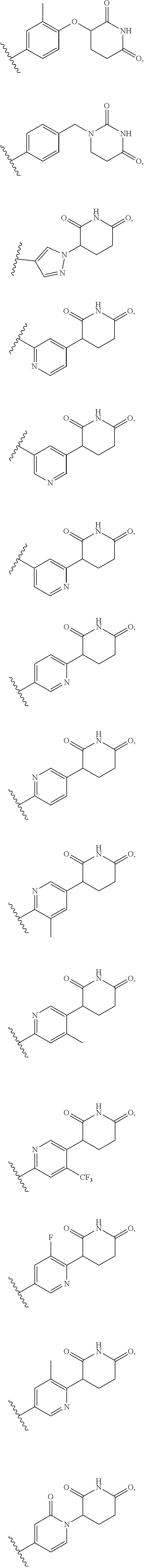

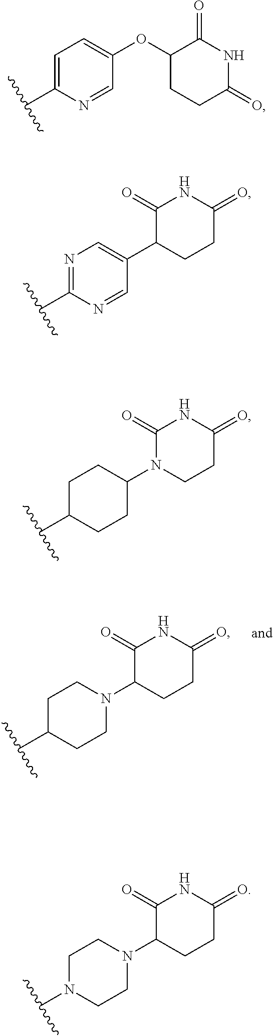 [SMILES: *c1ccc(OC2CCC(=O)NC2=O)cn1 ; *c1ncc(C2CCC(=O)NC2=O)cn1 ; *C1CCC(N2CCC(=O)NC2=O)CC1 ; *C1CCN(C2CCC(=O)NC2=O)CC1 ; *N1CCN(C2CCC(=O)NC2=O)CC1]

In some embodiments, R^{a} is halogen (e.g., —F, —Cl or —Br). In some embodiments, R^{a} is —CN. In some embodiments, R^{a} is —R′ as described herein. In some embodiments, R^{a} is —R as described herein. In some embodiments, R^{a} is —R but is not hydrogen. In some embodiments of any Formulae described herein, each Rₐ is independently hydrogen or optionally substituted C₁₋₆ aliphatic. In some embodiments, each R^{a} is independently hydrogen or optionally substituted C₁₋₆ alkyl. In some embodiments, each R^{a} is independently hydrogen, halogen, —CN, C₁₋₆ alkyl, or C₃₋₆ cycloalkyl. In some embodiments, each R^{a} is independently is hydrogen, halogen, —CN, C₁₋₆ alkyl, C₃₋₆ cycloalkyl, or C₆₋₁₀ aryl. In some embodiments, each R^{a} is independently hydrogen, halogen, —CN, C₁₋₆ alkyl, C₃₋₆ cycloalkyl, C₆₋₁₀ aryl, or 5- to 10-membered heteroaryl having 1-5 heteroatoms independently selected from N, O, and S. In some embodiments, each R^{a} is independently hydrogen, halogen, —CN, C₁₋₆ alkyl, C₃₋₆ cycloalkyl, C₆₋₁₀ aryl, 5- to 10-membered heteroaryl having 1-5 heteroatoms independently selected from N, O, and S, or 3- to 10-membered monocyclic heterocyclyl having 1-5 heteroatoms independently selected from N, O, and S. In some embodiments, each R^{a} is hydrogen. In some embodiments, R^{a} is hydrogen. In some embodiments, R^{a} is optionally substituted C₁₋₆ aliphatic. In some embodiments, R^{a} is optionally substituted C₁₋₆ alkyl. In some embodiments, R^{a} is C₁₋₆ alkyl. In some embodiments, R^{a} is optionally substituted C₁₋₂ alkyl. In some embodiments, R^{a} is C₁₋₂ alkyl (e.g., methyl). In some embodiments, R^{a} is optionally substituted C₃₋₆ cycloaliphatic. In some embodiments, R^{a} is optionally substituted C₃₋₆ cycloalkyl. In some embodiments, R^{a} is C₃₋₄ cycloalkyl (e.g., cyclopropyl). In some embodiments, R^{a} is C₆₋₁₀ aryl. In some embodiments, R^{a} is phenyl. In some embodiments, R^{a} is naphthyl. In some embodiments, R^{a} is 5- to 10-membered heteroaryl having 1-5 heteroatoms independently selected from N, O, and S. In some embodiments, R^{a} is 5- to 6-membered heteroaryl having 1-3 heteroatoms independently selected from N, O, and S. In some embodiments, R^{a} is 3- to 10-membered monocyclic heterocyclyl having 1-5 heteroatoms independently selected from N, O, and S. In some embodiments, R^{a} is 4- to 6-membered monocyclic heterocyclyl having 1-3 heteroatoms independently selected from N, O, and S.

In some embodiments, two R^{a} groups are each —R′ which are taken together with the atom to which they are attached to form an optionally substituted ring as described herein. In some embodiments, two R^{a} groups, together with the atom to which they are attached, combine to form a 3- to 6-membered saturated or partially unsaturated ring (e.g., a carbocycle or heterocycle having 1-2 heteroatoms independently selected from N, O, and S). In some embodiments, two R^{a} groups, together with the atom to which they are attached, combine to form a 3- to 6-membered saturated ring (e.g., a carbocycle or heterocycle having 1-2 heteroatoms independently selected from N, O, and S). In some embodiments, two R^{a} groups, together with the atom to which they are attached, combine to form a 3- to 4-membered saturated ring (e.g., a carbocycle or heterocycle having 1-2 heteroatoms independently selected from N, O, and S).

In some embodiments of any Formulae described herein, Y is N. In some embodiments, Y is CH.

In some embodiments, L² is a covalent bond. In some embodiments, L² is an optionally substituted C₁₋₃ hydrocarbon chain, wherein one or more methylene units are optionally and independently replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —S(O)—, —SO₂—, —SO₂N(R)—, —N(R)SO₂—, or -Cy-. In some embodiments, L² is an optionally substituted straight C₁₋₃ hydrocarbon chain, wherein one or more methylene units are optionally and independently replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —S(O)—, —SO₂—, —SO₂N(R)—, —N(R)SO₂—, or -Cy-. In some embodiments of any Formulae described herein, L² is a covalent bond or an optionally substituted straight or branched C₁₋₃ hydrocarbon chain. In some embodiments, L² is a covalent bond or a straight or branched C₁₋₃ hydrocarbon chain. In some embodiments, L² is a covalent bond or a straight or branched C₁₋₃ hydrocarbon chain wherein one methylene is replaced with —O—, —S—, —N(R)—, —SO₂—, —C(O)N(R)—, or —N(R)C(O)—. In some embodiments, L² is a covalent bond. In some embodiments, L² is a straight or branched C₁₋₃ hydrocarbon chain wherein one methylene is optionally replaced with —O—, —S—, —N(R)—, —SO₂—, —C(O)N(R)—, or —N(R)C(O)—. In some embodiments, L² is a C₁ hydrocarbon chain wherein one methylene is optionally replaced with —O—, —S—, —N(R)—, —SO₂—, —C(O)N(R)—, or —N(R)C(O)—. In some embodiments, L² is a straight or branched C₂ hydrocarbon chain wherein one methylene is optionally replaced with —O—, —S—, —N(R)—, —SO₂—, —C(O)N(R)—, or —N(R)C(O))—. In some embodiments, L² is a straight or branched C₃ hydrocarbon chain wherein one methylene is optionally replaced with O—, —S—, —N(R)—, —SO₂—, —C(O)N(R)—, or —N(R)C(O)—. In some embodiments, L² is a covalent bond, —CH—, —O—, or —N(R)—. In some embodiments, L² is —CH₂—. In some embodiments, L² is —CH₂—, —O—, or —N(R)—. In some embodiments, L² is —CH₂—, —O—, or —N(H)—. In some embodiments, L² is —O— or —S—. In some embodiments, L² is —O—. In some embodiments, L² is —S—. In some embodiments, L² is —N(R)—. In some embodiments, L² is —N(H)—. In some embodiments, L² is —SO₂—. In some embodiments, —C(O)N(R)—, or —N(R)C(O)—. In some embodiments, L² is —C(O)N(R)—. In some embodiments, L² is —N(R)C(O)—. In some embodiments, L² is —C(O)NH—. In some embodiments, L² is —NHC(O)—. In some embodiments, L² is —S(O)—. In some embodiments, L² is —S(O)N(R)—. In some embodiments, L² is —N(R)S(O)—. In some embodiments, L² is —S(O)NH—. In some embodiments, L² is —NHS(O)—. In some embodiments, L² is —S(O)₂—. In some embodiments, L² is —N(R)S(O)₂—. In some embodiments, L² is —S(O)₂N(R)—. In some embodiments, L² is —NHS(O)₂—. In some embodiments, L² is —S(O)₂NH—. In some embodiments, L² is -Cy- as described herein. In some embodiments, L¹ is -Cy- wherein -Cy- is aromatic.

In some embodiments, Ring E is

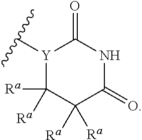

In some embodiments, Ring E is

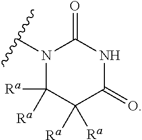

In some embodiments. Ring E is

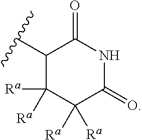

In some embodiments, Ring E is optionally substituted

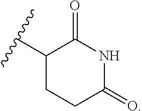 [SMILES: *C1CCC(=O)NC1=O]

In some embodiments, Ring E is

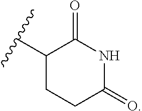 [SMILES: *C1CCC(=O)NC1=O]

In some embodiments, Ring E is optionally substituted

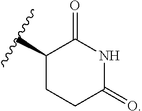 [SMILES: *[C@@H]1CCC(=O)NC1=O]

In some embodiments, Ring E is

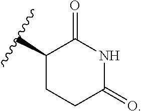 [SMILES: *[C@@H]1CCC(=O)NC1=O]

In some embodiments, Ring E is optionally substituted

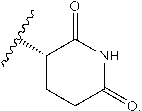 [SMILES: *[C@H]1CCC(=O)NC1=O]

In some embodiments, Ring E is

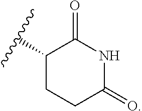 [SMILES: *[C@H]1CCC(=O)NC1=O]

In some embodiments, one stereoisomeric form binds an E3 ubiquitin ligase stronger than the other. In some embodiments, an stereoisomeric form can convert into the other, e.g., during storage, upon administration, after administration, etc. In some embodiments, a compound can be provided or administered either as a stereochemically pure form or as a mixture of two stereoisomers with respect to the carbon at which Ring E is bonded to L² or Ring C. In some embodiments, stereochemical purity of a compound in a composition with respect to the carbon center at which Ring E is bonded to L² or Ring C is about or at least about 5%, 10%, 15%, 20%, 25%, 30%, 40%, 50%, 60%, 70%, 75%, 80%, 85%, 90%, or 95%. In some embodiments, stereochemical purity of a compound in a composition with respect to any other chiral center is about or at least about 90% or 95%.

In some embodiments, Ring E is optionally substituted

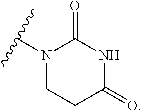 [SMILES: *N1CCC(=O)NC1=O]

In some embodiments, Ring E is

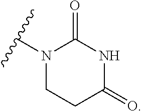 [SMILES: *N1CCC(=O)NC1=O]

In some embodiments of any Formulae described herein, Ring Cis an optionally substituted, mono- or bicyclic, 3- to 10-membered bivalent ring system, wherein the ring system is fully saturated, partially saturated, or aromatic, and the ring system contains 0-4 heteroatoms independently selected from N, O, and S. In some embodiments, Ring C is an optionally substituted group selected from phenyl. C₅₋₆ cycloaliphatic, 5- to 6-membered heteroaryl having 1-4 heteroatoms independently selected from N, O, and S, 5- to 6-membered heterocyclyl having 1-2 heteroatoms independently selected from N, O, and S, and 9- to 10-membered bicyclic heteroaryl having 1-4 heteroatoms independently selected from N, O, and S. In some embodiments, Ring C is an optionally substituted group selected from phenyl, 5- to 6-membered heteroaryl having 1-4 heteroatoms independently selected from N, O, and S, and 9- to 10-membered bicyclic heteroaryl having 1-4 heteroatoms independently selected from N, O, and S. In some embodiments, Ring C is optionally substituted phenyl or optionally substituted 5- to 6-membered heteroaryl having 1-4 heteroatoms independently selected from N, O, and S. In some embodiments, Ring C is optionally substituted C₃-C₇ cycloaliphatic or optionally substituted 3- to 7-membered heterocyclyl having 1-2 heteroatoms independently selected from N, O, and S.

In some embodiments, Ring C is an optionally substituted 3-16 (e.g., 3, 4, 5, 6, 7, 8, 9, 10, 11, 12, 13, 14, 15, 16, 3-15, 3-10, 5-10, 3-8, 3-7, or 5-6) membered ring having 0-6 (e.g., 0, 1-6, 1-4, 1, 2, 3, 4, 5, or 6) heteroatoms independently selected from N, O, and S.

In some embodiments, Ring C is optionally substituted phenyl ring. In some embodiments, Ring C is a phenyl ring. In some embodiments, Ring C is optionally substituted 5- to 6-membered heteroaryl having 1-4 heteroatoms independently selected from N, O, and S. In some embodiments. Ring C is optionally substituted 5- to 6-membered heteroaryl having 1-2 heteroatoms independently selected from N, O, and S. In some embodiments, Ring C is optionally substituted 5-membered heteroaryl having 1-2 heteroatoms independently selected from N, O, and S. In some embodiments, Ring C is a pyrrole. In some embodiments, Ring C is optionally substituted 6-membered heteroaryl having 1-2 heteroatoms independently selected from N, O, and S. In some embodiments, Ring C is a pyridine, pyridone, or pyrimidine.

In some embodiments, Ring C is optionally substituted C₃-C₇ cycloaliphatic. In some embodiments, Ring C is optionally substituted C₃-C₇ cycloalkyl. In some embodiments, Ring C is optionally substituted C₅-C₆ cycloaliphatic. In some embodiments, Ring C is optionally substituted C₅-C₆ cycloalkyl. In some embodiments, Ring C is a cyclohexane.

In some embodiments, Ring C is optionally substituted 3- to 7-membered heterocyclyl having 1-2 heteroatoms independently selected from N, O, and S. In some embodiments, Ring C is optionally substituted 4- to 6-membered heterocyclyl having 1-2 heteroatoms independently selected from N, O, and S. In some embodiments, Ring C is a 6-membered heterocyclyl having 1-2 heteroatoms independently selected from N, O, and S. In some embodiments, Ring C is a piperidine or piperazine.

In some embodiments, Ring C is optionally substituted 9- to 10-membered bicycle heteroaryl having 1-4 heteroatoms independently selected from N, O, and S. In some embodiments, Ring C is optionally substituted 9-membered bicyclic heteroaryl having 1-4 heteroatoms independently selected from N, O, and S. In some embodiments, Ring C is optionally substituted phthalimide, isoindolin-1-one, indazole, benzo[d][1,2,3]triazole, benzo[d]oxazol-2 (3H)-one, 1,3-dihydro-2H-benzo[d]imidazole-2-one, or isoquinoline. In some embodiments, Ring C is optionally substituted phthalimide or isoindolin-1-one. In some embodiments, Ring C is optionally substituted 10-membered bicyclic heteroaryl having 1-4 heteroatoms independently selected from N, O, and S. For example, in some embodiments, Ring C is optionally substituted

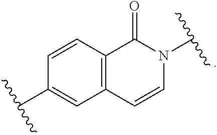 [SMILES: *c1ccc2c(=O)n(*)ccc2c1]

In some embodiments, Ring C is

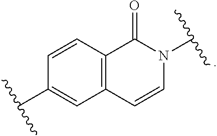 [SMILES: *c1ccc2c(=O)n(*)ccc2c1]

In some embodiments, the carbon atom is bonded to linker. In some embodiments, Ring C is optionally substituted

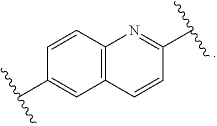 [SMILES: *c1ccc2nc(*)ccc2c1]

In some embodiments, Ring C is

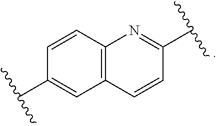 [SMILES: *c1ccc2nc(*)ccc2c1]

In some embodiments, the carbon atom in the phenyl ring is bonded to linker.

In some embodiments, Ring C is optionally substituted 10- to 16-membered polycyclic heteroaryl having 1-4 heteroatoms independently selected from N, O, and S. In some embodiments. Ring C is an optionally substituted 11-membered tricyclic heteroaryl having 1-4 heteroatoms independently selected from N, O, and S (e.g., 6,7-dihydropyrrolo[3,4-f]isoindole-1,3(2H,5H)-dione).

In some embodiments, Ring C is

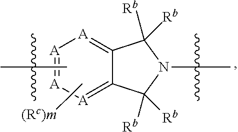

wherein:
each A is independently N, C, or CH, provided that no more than two A groups are N;
each R^{b} is hydrogen, or two R^{b} groups, on the same carbon, are taken together to form an oxo or combine to form a 3- to 6-membered saturated or partially unsaturated ring;
each R^{c} is independently selected from halogen, —OR, —N(R)₂, —CN, and optionally substituted C₁₋₆ aliphatic;
m is 0, 1, 2, or 3.

In some embodiments, Ring C is

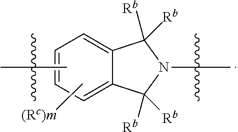

In some embodiments, Ring C is selected from:

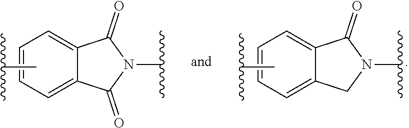

In some embodiments, Ring C is

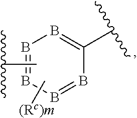

wherein:
each B is independently selected from N, C, and CH, provided that no more than two B are N;
each R^{c} is independently selected from halogen, —OR, —N(R)₂, —CN, and optionally substituted C₁₋₆ aliphatic; and
m is 0, 1, 2, or 3.

In some embodiments, Ring C is

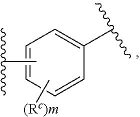

wherein:
each R^{c} is independently selected from halogen, —OR, —N(R)₂, —CN, and optionally substituted C₁₋₆ aliphatic; and
m is 0, 1, 2, or 3.

In some embodiments, Ring C is selected from:

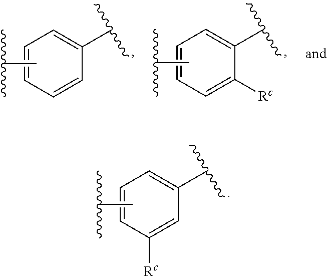

In some embodiments, Ring C is selected from:

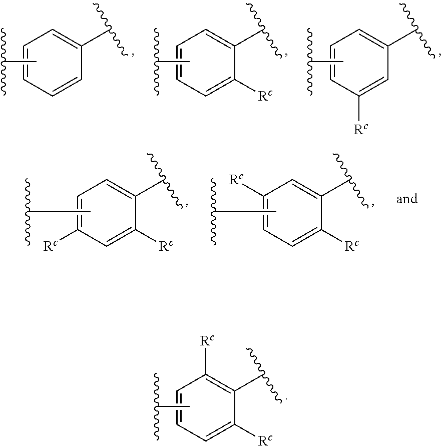

In some embodiments, Ring C is optionally substituted

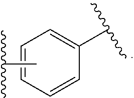

In some embodiments, Ring C is optionally substituted

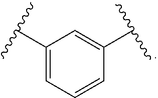 [SMILES: *c1cccc(*)c1]

In some embodiments, Ring C is

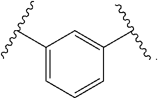 [SMILES: *c1cccc(*)c1]

In some embodiments, Ring C is optionally substituted

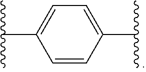 [SMILES: *c1ccc(*)cc1]

In some embodiments, Ring C is

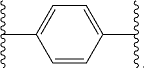 [SMILES: *c1ccc(*)cc1]

In some embodiments, Ring C has a substituent at a position next to the position (e.g., o for a phenyl ring) bonded to L² (as those skilled in the art appreciate, Ring E when L² is a covalent bond). For example, in some embodiments, a substituent at a position next to the position bonded to L² (e.g., R^{b}, R^{b1}), or R^{c}) is —OR, —R or halogen. In some embodiments, it is —OR wherein R is as described herein. In some embodiments, it is —OR, wherein R is optionally substituted C₁₋₄ aliphatic. In some embodiments, it is —OR wherein R is optionally substituted C₁₋₄ alkyl. In some embodiments, it is —OR wherein R is C₁₋₄ aliphatic. In some embodiments, it is —OR wherein R is C₁₋₄ alkyl. In some embodiments, it is —OR wherein R is C₁₋₄ haloalkyl. In some embodiments, it is —OCF₃. In some embodiments, it is —OMe. In some embodiments, it is —R as described herein. In some embodiments, it is optionally substituted C₁₋₄ aliphatic. In some embodiments, it is optionally substituted C₁₋₄ alkyl. In some embodiments, it is C₁₋₄ aliphatic. In some embodiments, it is C₁₋₄ alkyl. In some embodiments, it is C₁₋₄ haloalkyl. In some embodiments, it is methyl. In some embodiments, it is —CF₃. In some embodiments, it is halogen. In some embodiments, it is —F. In some embodiments, it is —Cl. In some embodiments, it is —Br. For example, in some embodiments, Ring C is

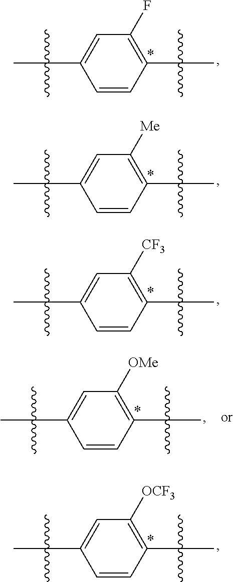

wherein the carbon atom marked with * is bonded to L². In some embodiments, Ring C is a bicyclic or polycyclic ring, and Ring C has two ring atoms bonded to a position next to the position bonded to L² (as those skilled in the art appreciate, Ring E when L² is a covalent bond). For examples, in some embodiments, Ring C is

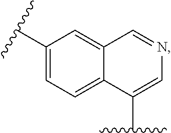 [SMILES: *c1ccc2c(*)cncc2c1]

wherein the carbon atom marked with * is bonded to L². In some embodiments,

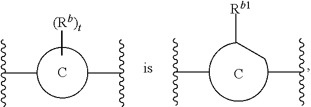

wherein R^{b1} is not hydrogen. In some embodiments, compounds comprising such Ring C can selectively degrade KAT2A over KAT2B (e.g., DC₅₀ for KAT2A is smaller than that for KAT2B when assessed, e.g., as in the Examples such as Example B1).

In some embodiments, Ring C is selected from:

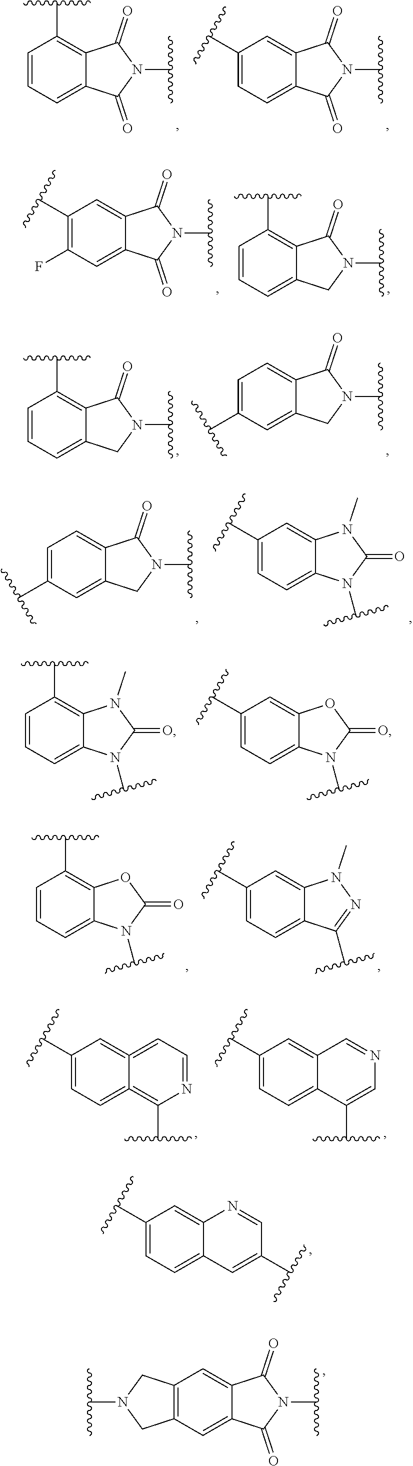

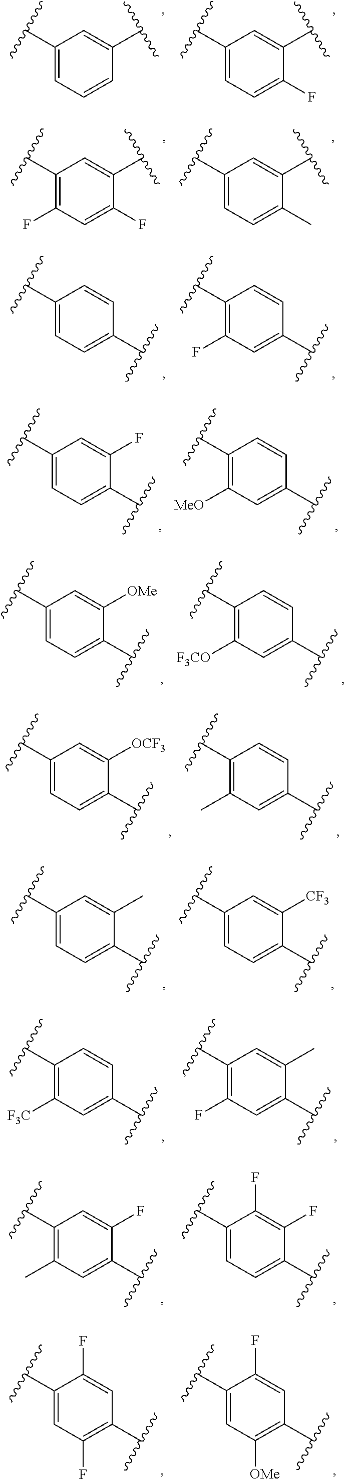

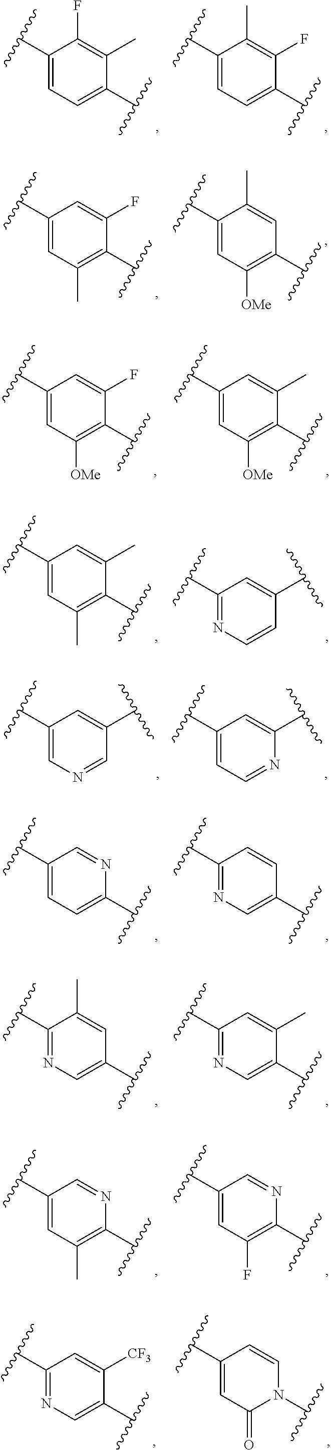

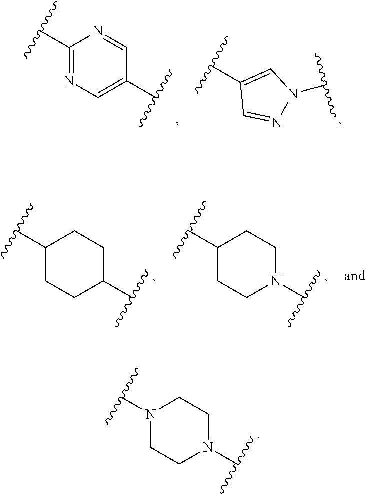 [SMILES: *c1cnc(*)nc1 ; *c1cnn(*)c1 ; *C1CCC(*)CC1 ; *C1CCN(*)CC1 ; *N1CCN(*)CC1]

In some embodiments, Ring C is selected from:

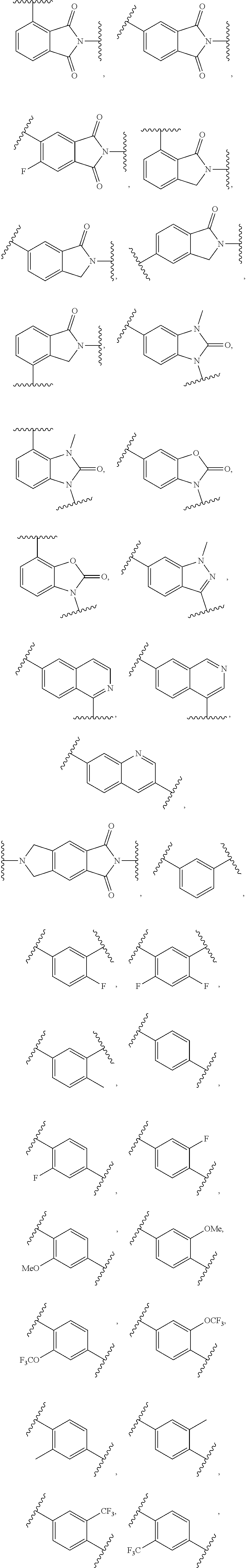

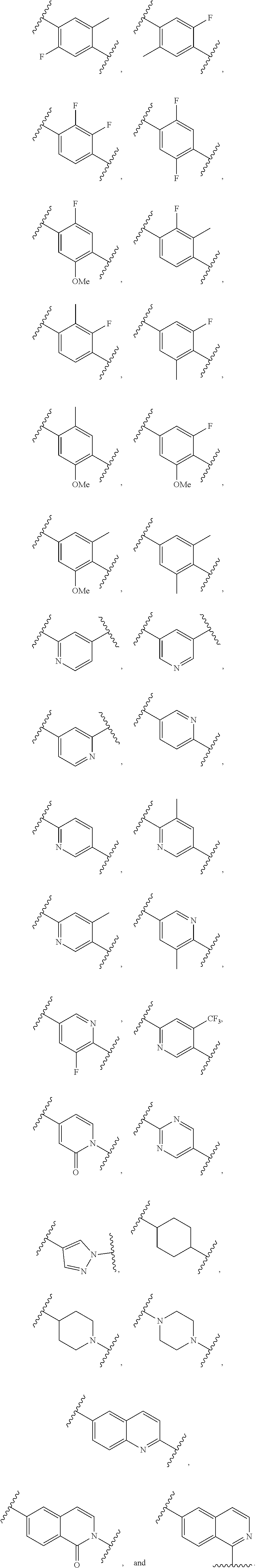

In some embodiments of any Formulae described herein, Ring F is an optionally substituted 3- to 16-membered ring having 0-6 heteroatoms independently selected from N, O, and S. In some embodiments. Ring F is an optionally substituted 4- to 10-membered bivalent ring system having 0-4 heteroatoms independently selected from N, O, and S. In some embodiments, Ring F is an optionally substituted 5- to 6-membered bivalent ring system having 0-3 heteroatoms independently selected from N, O, and S. In some embodiments, Ring F is an optionally substituted 6-membered bivalent ring system having 0-2 heteroatoms independently selected from N, O, and S. In some embodiments, Ring F is an optionally substituted phenyl.

In some embodiments of any Formulae described herein, each A is CH. In some embodiments, one A is N and the other A groups are CH. In some embodiments, two A groups are N, and the other A groups are CH. It will be appreciated that when A is CH, it may be substituted with R^{c}, as defined herein, such that the ring contains a —C(R^{c})— moiety. It will also be appreciated that A is C, when it is the point of attachment to the rest of the molecule.

In some embodiments, R^{b} is R′ as described herein. In some embodiments, R^{b} is —R as described herein. In some embodiments, R^{b} is —R but not hydrogen. In some embodiments, R^{b} is —OR wherein R is as described herein. In some embodiments of any Formulae described herein, each R^{b} is hydrogen. In some embodiments of any Formulae described herein, each R^{b} is independently halogen, —CN, —R or —OR. In some embodiments, each R^{b} is independently hydrogen, halogen, or optionally substituted C₁₋₆ aliphatic. In some embodiments, each R^{b} is independently hydrogen, halogen, —CN, C₁₋₆ alkyl, or C₃₋₆ cycloalkyl. In some embodiments, each R^{b} is independently is hydrogen, halogen, —CN, C₁₋₆ alkyl, C₃₋₆ cycloalkyl, or C₆₋₁₀ aryl. In some embodiments, each R^{b} is independently hydrogen, halogen, —CN, C₁₋₆ alkyl, C₃₋₆ cycloalkyl, C₆₋₁₀ aryl, or 5- to 10-membered heteroaryl having 1-5 heteroatoms independently selected from N, O, and S. In some embodiments, each R^{b} is independently hydrogen, halogen, —CN, C₁₋₆ alkyl, C₃₋₆ cycloalkyl, C₆₋₁₀ aryl, 5- to 10-membered heteroaryl having 1-5 heteroatoms independently selected from N, O, and S, or 3- to 10-membered monocyclic heterocyclyl having 1-5 heteroatoms independently selected from N, O, and S. In some embodiments, each R^{b} is independently hydrogen. In some embodiments, Rbis halogen. In some embodiments, R^{b} is fluoro. In some embodiments, R^{b} is chloro. In some embodiments, R^{b} is bromo. In some embodiments, R^{b} is optionally substituted C₁₋₆ aliphatic. In some embodiments, R^{b} is optionally substituted C₁₋₆ alkyl. In some embodiments, R^{b} is C₁₋₂ alkyl (e.g., methyl). In some embodiments, R^{b} is optionally substituted C₃₋₆ cycloaliphatic. In some embodiments, R^{b} is optionally substituted C₃₋₆ cycloalkyl. In some embodiments, R^{b} is C₃₋₄ cycloalkyl (e.g., cyclopropyl). In some embodiments, R^{b} is C₆₋₁₀ aryl. In some embodiments, R^{b} is phenyl. In some embodiments, R^{b} is naphthyl. In some embodiments, R^{b} is 5- to 10-membered heteroaryl having 1-5 heteroatoms independently selected from N, O, and S. In some embodiments. R^{b} is 5- to 6-membered heteroaryl having 1-3 heteroatoms independently selected from N, O, and S. In some embodiments, R^{b} is 3- to 10-membered monocyclic heterocyclyl having 1-5 heteroatoms independently selected from N, O, and S. In some embodiments, R^{b} is 4- to 6-membered monocyclic heterocyclyl having 1-3 heteroatoms independently selected from N, O, and S.

In some embodiments, two R^{b} groups, on the same carbon, are taken together to form an oxo. In some embodiments, two R^{b} groups, on the same carbon, combine to form a 3- to 6-membered saturated or partially unsaturated ring (e.g., a C₃₋₆ cycloaliphatic or 3- to 6-membered heterocyclyl having 1-2 heteroatoms independently selected from N, O, and S). In some embodiments, two R^{b} groups, on the same carbon, combine to form C₃₋₆ cycloalkyl (e.g., cyclopropyl). In some embodiments, two R^{b} groups, on the same carbon, combine to form a 3- to 6-membered heterocyclyl having 1-2 heteroatoms independently selected from N, O, and S.

In some embodiments, R^{b1} is R^{b} as described herein. In some embodiments, R^{b1} is not hydrogen. In some embodiments, it is —OR wherein R is as described herein. In some embodiments, it is —OR wherein R is optionally substituted C₁₋₄ aliphatic. In some embodiments, it is —OR wherein R is optionally substituted C₁₋₄ alkyl. In some embodiments, it is —OR wherein R is C₁₋₄ aliphatic. In some embodiments, it is —OR wherein R is C₁₋₄ alkyl. In some embodiments, it is —OR wherein R is C₁₋₄ haloalkyl. In some embodiments, it is —OCF₃. In some embodiments, it is —OMe. In some embodiments, it is —R as described herein. In some embodiments, it is optionally substituted C₁₋₄ aliphatic. In some embodiments, it is optionally substituted C₁₋₄ alkyl. In some embodiments, it is C₁₋₄ aliphatic. In some embodiments, it is C₁₋₄ alkyl. In some embodiments, it is C₁₋₄ haloalkyl. In some embodiments, it is methyl. In some embodiments, it is —CF₃. In some embodiments, it is halogen. In some embodiments, it is —F. In some embodiments, it is —Cl. In some embodiments, it is —Br.

In some embodiments, t′ is 0. In some embodiments, t′ is 1. In some embodiments, t′ is 2. In some embodiments, t′ is 3.

In some embodiments of any Formulae described herein, each B is CH, C—R^{c}, or C. In some embodiments, one B is N and the other B groups are CH or C. In some embodiments, two B groups are N and the other B groups are CH or C. In some embodiments, B is N. In some embodiments, B is CH. In some embodiments, B is C—R^{c}. In some embodiments, B is C. It will be appreciated that B is C, when it is the point of attachment to the rest of the molecule.

In some embodiments of any Formulae described herein, each R^{c} is independently selected from halogen, —OH, —O(C₁₋₆ alkyl), —O(C₁₋₆ haloalkyl), —NH₂, —NH(C₁₋₆ alkyl), —N(C₁₋₆ alkyl)₂, —CN, and optionally substituted C₁₋₆ alkyl. In some embodiments, each R^{c} is independently selected from halogen, —OH, —O(C₁₋₆ alkyl), —NH₂, —NH(C₁₋₆ alkyl), —N(C₁₋₆ alkyl)₂, —CN, and optionally substituted C₁₋₆ alkyl. In some embodiments, each R^{c} is independently selected from halogen, —O(C₁₋₆ alkyl), —O(C₁₋₆ haloalkyl), C₁₋₆ alkyl, and C₁₋₆ haloalkyl. In some embodiments, each R^{c} is independently selected from halogen, —O(C₁₋₆ alkyl), C₁₋₆ alkyl, and C₁₋₆ haloalkyl. In some embodiments, each R^{c} is independently selected from halogen, —O(C₁₋₆ alkyl), and C₁₋₆ alkyl. In some embodiments, R^{c} is halogen (e.g., fluoro or chloro). In some embodiments, R^{c} is —OR (e.g., —OH, —O(C₁₋₆ alkyl), or —O(C₁₋₆ haloalkyl)). In some embodiments, R^{c} is —OCH₃ or —OCF₃. In some embodiments, R^{c} is —N(R)₂ (e.g., —NH₂, NH(C₁₋₆ alkyl), or —N(C₁₋₆ alkyl)₂). In some embodiments, R^{c} is —CN. In some embodiments, R^{c} is optionally substituted C₁₋₆ aliphatic. In some embodiments, R^{c} is optionally substituted C₁₋₆ alkyl. In some embodiments. R^{c} is C₁₋₆ alkyl optionally substituted with one or more halogen. In some embodiments, R^{c} is C₁₋₆ alkyl. In some embodiments, R^{c} is optionally substituted C₁₋₂ alkyl. In some embodiments, R^{c} is optionally substituted C₁₋₂ alkyl optionally substituted with one or more halogen. In some embodiments. R^{c} is C₁₋₂ alkyl (e.g., methyl). In some embodiments, R^{c} is C₁₋₆ haloalkyl. In some embodiments. R^{c} is C₁₋₂ haloalkyl (e.g., —CF₃).

In some embodiments of any Formulae described herein, m is 0, 1, or 2. In some embodiments, m is 0 or 1. In some embodiments, m is 0. In some embodiments, m is 1. In some embodiments, m is 2. In some embodiments, m is 3.

In some embodiments of any Formulae described herein, a LBM is a Von Hippel-Lindau protein (VHL) binding moiety, i.e., is a moiety that is capable of binding Von Hippel-Lindau protein.

In some embodiments, LBM is

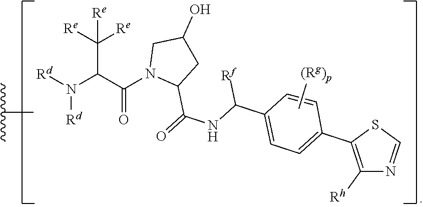

In some embodiments of any Formulae described herein, LBM has the following structure:

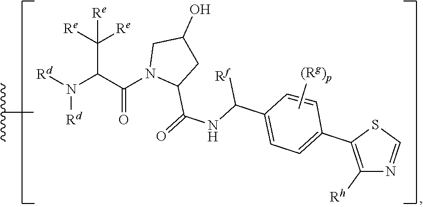

wherein R^{d}, R^{e}, R^{f}, R^{g}, R^{h}, and p are as defined herein for Formula IV and described in classes and subclasses herein, both singly and in combination.

In some embodiments of any Formulae described herein, a LBM is selected from:

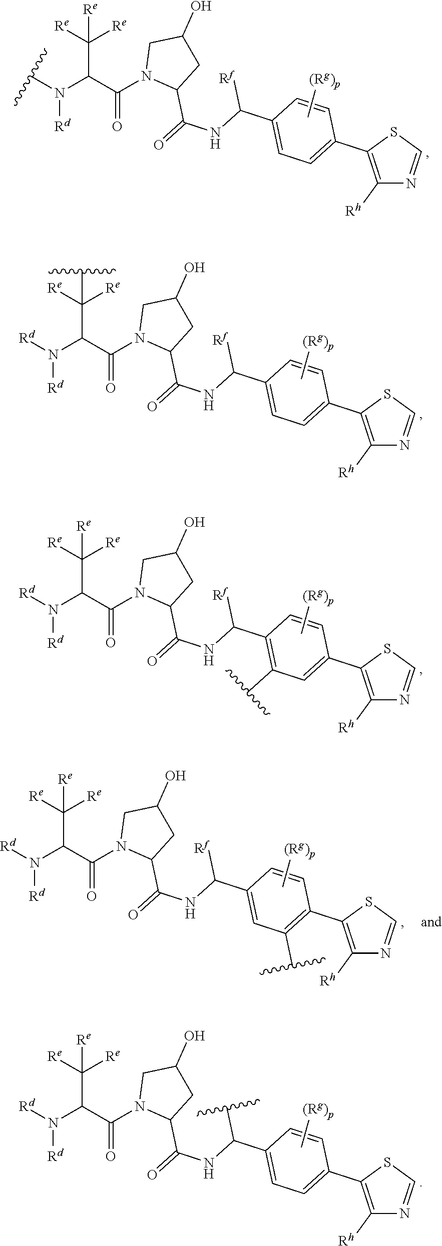

In some embodiments, R^{d}, R^{e}, R^{f}, R^{g}, R^{h}, and p are as defined herein for Formula IV and described in classes and subclasses herein, both singly and in combination.

In some embodiments of any Formulae described herein, a LBM has the following structure:

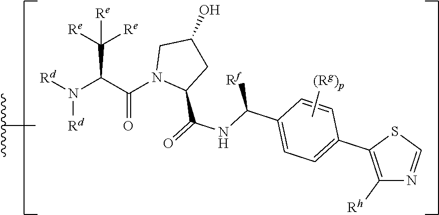

In some embodiments, R^{d}, R^{e}, R^{f}, R^{g}, R^{h}, and p are as defined herein for Formula IV and described in classes and subclasses herein, both singly and in combination.

In some embodiments of any Formulae described herein, a LBM has the following structure:

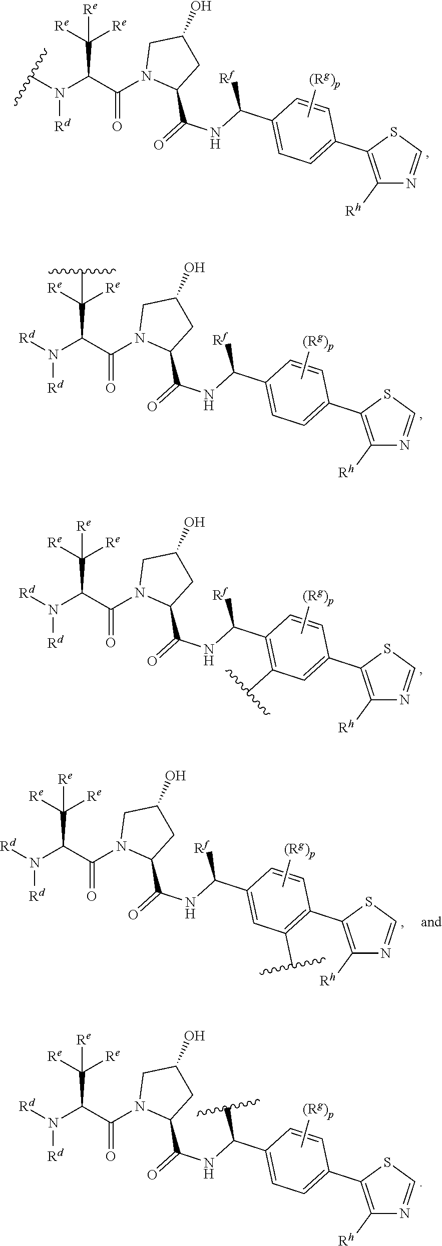

In some embodiments, R^{d}, R^{e}, R^{f}, R^{g}, R^{h}, and p are as defined herein for Formula IV and described in classes and subclasses herein, both singly and in combination.

In some embodiments of any Formulae described herein, a LBM has the following structure:

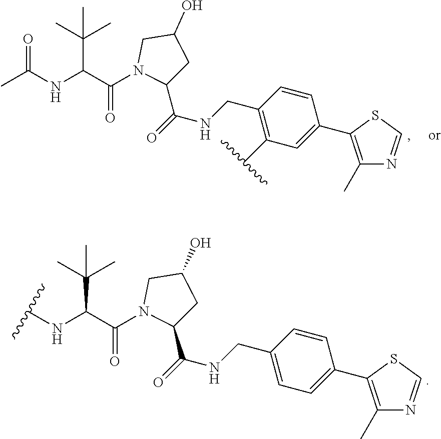 [SMILES: *c1cc(-c2scnc2C)ccc1CNC(=O)C1CC(O)CN1C(=O)C(NC(C)=O)C(C)(C)C ; *N[C@H](C(=O)N1C[C@H](O)C[C@H]1C(=O)NCc1ccc(-c2scnc2C)cc1)C(C)(C)C]

In some embodiments of any Formulae described herein, a LBM moiety (e.g., VHL binding moiety) is attached to linker at a R^{d}. In some embodiments, a LBM moiety (e.g., VHL binding moiety) is attached to linker at a R^{e}. In some embodiments, a LBM moiety (e.g., VHL binding moiety) is attached to linker at R^{f}. In some embodiments, a LBM moiety (e.g., VHL binding moiety) is attached to linker at a R^{g}.

In some embodiments, R^{d} is —R′ as described herein. In some embodiments, R^{d} is —R as described herein. In some embodiments, R^{d} is —R but is not hydrogen. In some embodiments of any Formulae described herein, each R^{d} that is not the point of attachment for the linker is independently hydrogen, —C(O)R, or optionally substituted C₁₋₆ aliphatic. In some embodiments, each R^{d} is hydrogen, optionally substituted C₁₋₆ aliphatic, —R, —C(O)R, or —S(O)₂R, or two R′ attached to the same atom, together with the atom to which they are attached, combine to form an optionally substituted 3-16 membered ring having 1-5 heteroatoms independently selected from N, O, and S. In some embodiments, R^{d} is hydrogen. In some embodiments, R^{d} is —C(O)R. In some embodiments, R^{d} is —C(O)(optionally substituted C₁₋₆ alkyl). In some embodiments, R^{d} is —C(O)(optionally substituted C₃₋₆ cycloalkyl). In some embodiments, R^{d} is optionally substituted C₁₋₆ aliphatic. In some embodiments, R^{d} is optionally substituted C₁₋₆ alkyl. In some embodiments, R^{d} is C₁₋₆ alkyl. In some embodiments, R^{d} is optionally substituted C₁₋₂ alkyl. In some embodiments, R^{d} is C₁₋₆ alkyl (e.g., methyl). In some embodiments, each R^{d} is optionally substituted C₃₋₆ cycloaliphatic. In some embodiments, each R^{d} is optionally substituted C₃₋₆ cycloalkyl. In some embodiments, each R^{d} is C₃₋₄ cycloalkyl (e.g., cyclopropyl). In some embodiments, each R^{d} is —C(O)R. In some embodiments, each R^{d} is —C(O)(C₁₋₆ alkyl). In some embodiments, each R^{d} is —S(O)₂R. In some embodiments, each R^{d} is —S(O)₂(C₁₋₆ alkyl). In some embodiments, each R^{d} is two R′ attached to the same atom, together with the atom to which they are attached, combined to form an optionally substituted 3-16 membered ring having 1-5 heteroatoms independently selected from N, O, and S.

In some embodiments, two R^{d} are each R′ and the two R′ are taken together with the nitrogen to which they are bonded to form a ring as described herein. In some embodiments, two R^{d}, together with the atom to which they are attached, combine to form an optionally substituted 5- to 6-membered ring having 1-3 heteroatoms independently selected from N, O, and S and optionally fused to a phenyl or 5- to 6-membered heteroaryl ring having 1-3 heteroatoms independently selected from N, O, and S. In some embodiments, two R^{d}, together with the atom to which they are attached, combine to form an optionally substituted 5- to 6-membered ring having 1-3 heteroatoms independently selected from N, O, and S. In some embodiments, two R^{d}, together with the atom to which they are attached, combine to form an optionally substituted 5- to 6-membered ring having 1-3 heteroatoms independently selected from N, O, and S that is fused to a phenyl or 5- to 6-membered heteroaryl ring having 1-3 heteroatoms independently selected from N, O, and S.

In some embodiments, R^{e} is —R as described herein. In some embodiments, R^{e} is —R but is not hydrogen. In some embodiments of any Formulae described herein, each R^{e} that is not the point of attachment for the linker is independently hydrogen or optionally substituted C₁₋₆ alkyl. In some embodiments, each R^{e} is optionally substituted C₁₋₆ aliphatic. In some embodiments, each R^{e} is optionally substituted C₁₋₆ alkyl (e.g., methyl). In some embodiments, R^{e} is hydrogen. In some embodiments, R^{e} is optionally substituted C₁₋₆ aliphatic. In some embodiments, R^{e} is optionally substituted C₁₋₆ alkyl. In some embodiments, R^{e} is C₁₋₆ alkyl. In some embodiments, R^{e} is optionally substituted C₁₋₂ alkyl. In some embodiments, R^{e} is C₁₋₂ alkyl (e.g., methyl). In some embodiments of any Formulae described herein, each R^{e} that is not the point of attachment for the linker is independently —R. In some embodiments, each R^{e} is independently hydrogen, halogen, or optionally substituted C₁₋₆ aliphatic. In some embodiments, each R^{e} is independently hydrogen, halogen, —CN, C₁₋₆ alkyl, or C₃₋₆ cycloalkyl. In some embodiments, each R^{e} is independently is hydrogen, halogen, —CN, C₁₋₆ alkyl, C₃₋₆ cycloalkyl, or C₆₋₁₀ aryl. In some embodiments, each R^{e} is independently hydrogen, halogen, —CN, C₁₋₆ alkyl, C₃₋₆ cycloalkyl, C₆₋₁₀ aryl, or 5- to 10-membered heteroaryl having 1-5 heteroatoms independently selected from N, O, and S. In some embodiments, each R^{e} is independently hydrogen, halogen, —CN, C₁₋₆ alkyl, C₃₋₆ cycloalkyl, C₆₋₁₀ aryl, 5- to 10-membered heteroaryl having 1-5 heteroatoms independently selected from N, O, and S, or 3- to 10-membered monocyclic heterocyclyl having 1-5 heteroatoms independently selected from N, O, and S. In some embodiments. R^{e} is halogen. In some embodiments, R^{e} is fluoro. In some embodiments, R^{e} is chloro. In some embodiments, R^{e} is bromo. In some embodiments, R^{e} is optionally substituted C₁₋₆ alkyl. In some embodiments, R^{e} is C₁₋₂ alkyl (e.g., methyl). In some embodiments, R^{e} is optionally substituted C₃₋₆ cycloaliphatic. In some embodiments, R^{e} is optionally substituted C₃₋₆ cycloalkyl. In some embodiments, R^{e} is C₃₋₄ cycloalkyl (e.g., cyclopropyl). In some embodiments. R^{e} is C₆₋₁₀ aryl. In some embodiments, R^{e} is phenyl. In some embodiments, R^{e} is naphthyl. In some embodiments, R^{e} is 5- to 10-membered heteroaryl having 1-5 heteroatoms independently selected from N, O, and S. In some embodiments, R^{e} is 5- to 6-membered heteroaryl having 1-3 heteroatoms independently selected from N, O, and S. In some embodiments, R^{e} is 3- to 10-membered monocyclic heterocyclyl having 1-5 heteroatoms independently selected from N, O, and S. In some embodiments, R^{e} is 4- to 6-membered monocyclic heterocyclyl having 1-3 heteroatoms independently selected from N, O, and S.

In some embodiments, R^{f} is —R as described herein. In some embodiments, R^{f} is —R but is not hydrogen. In some embodiments of any Formulae described herein. R^{f}, when it is not the point of attachment for the linker, is hydrogen or optionally substituted C₁₋₆ alkyl. In some embodiments, R^{f} is hydrogen. In some embodiments, R^{f} is optionally substituted C₁₋₆ aliphatic. In some embodiments, R^{f} is optionally substituted C₁₋₆ alkyl. In some embodiments, R^{f} is C₁₋₆ alkyl. In some embodiments, R^{f} is optionally substituted C₁₋₂ alkyl. In some embodiments, R^{f} is C₁₋₂ alkyl (e.g., methyl). In some embodiments of any Formulae described herein, R^{f}, when it is not the point of attachment for the linker, is —R. In some embodiments, each R^{f} is independently hydrogen, halogen, or optionally substituted C₁₋₆ aliphatic. In some embodiments, each R^{f} is independently hydrogen, halogen, —CN, C₁₋₆ alkyl, or C₃₋₆ cycloalkyl. In some embodiments, each R^{f} is independently is hydrogen, halogen, —CN, C₁₋₆ alkyl, C₃-6 cycloalkyl, or C₆₋₁₀ aryl. In some embodiments, each R^{f} is independently hydrogen, halogen, —CN, C₁₋₆ alkyl, C₃₋₆ cycloalkyl, C₆₋₁₀ aryl, or 5- to 10-membered heteroaryl having 1-5 heteroatoms independently selected from N, O, and S. In some embodiments, each R^{f} is independently hydrogen, halogen, —CN, C₁₋₆ alkyl, C₃₋₆ cycloalkyl, Cete aryl, 5- to 10-membered heteroaryl having 1-5 heteroatoms independently selected from N, O, and S, or 3- to 10-membered monocyclic heterocyclyl having 1-5 heteroatoms independently selected from N, O, and S. In some embodiments, R^{f} is halogen. In some embodiments, R^{f} is fluoro. In some embodiments, R^{f} is chloro. In some embodiments, R^{f} is bromo. In some embodiments, R^{f} is optionally substituted C₁₋₆ aliphatic. In some embodiments. R^{f} is C₁₋₂ alkyl (e.g., methyl). In some embodiments. R^{f} is optionally substituted C₃₋₆ cycloaliphatic. In some embodiments, R^{f} is optionally substituted C₃₋₆ cycloalkyl. In some embodiments, R^{f} is C₃₋₄ cycloalkyl (e.g., cyclopropyl). In some embodiments, R^{f} is C₆₋₁₀ aryl. In some embodiments, R^{f} is phenyl. In some embodiments, R^{f} is naphthyl. In some embodiments, R^{f} is 5- to 10-membered heteroaryl having 1-5 heteroatoms independently selected from N, O, and S. In some embodiments, R^{f} is 5- to 6-membered heteroaryl having 1-3 heteroatoms independently selected from N, O, and S. In some embodiments, R^{f} is 3- to 10-membered monocyclic heterocyclyl having 1-5 heteroatoms independently selected from N, O, and S. In some embodiments, R^{f} is 4- to 6-membered monocyclic heterocyclyl having 1-3 heteroatoms independently selected from N, O, and S.

In some embodiments, R^{g} is —R as described herein. In some embodiments, R^{g} is —R but is not hydrogen. In some embodiments, R^{g} is —OR wherein R is as described herein. In some embodiments of any Formulae described herein, each R^{g} that is not the point of attachment for the linker is independently halogen, —OR, —CN, or optionally substituted C₁₋₆ alkyl. In some embodiments, R^{g} is halogen (e.g., fluoro or chloro). In some embodiments, R^{g} is —OR (e.g., —OH or —O(C₁₋₆ alkyl)). In some embodiments, R^{g} is —CN. In some embodiments. R^{g} is optionally substituted C₁₋₆ aliphatic. In some embodiments, R^{g} is optionally substituted C₁₋₆ alkyl. In some embodiments, R^{g} is Cie alkyl. In some embodiments, R^{g} is optionally substituted C₁₋₂ alkyl. In some embodiments, R^{g} is C₁₋₂ alkyl (e.g., methyl). In some embodiments of any Formulae described herein, each R^{g} that is not the point of attachment for the linker is independently halogen, —CN, R or —OR. In some embodiments, each R^{g} is independently hydrogen, halogen, or optionally substituted C₁₋₆ aliphatic. In some embodiments, each R^{g} is independently hydrogen, halogen, —CN, C₁₋₆ alkyl, or C₃₋₆ cycloalkyl. In some embodiments, each R^{g} is independently is hydrogen, halogen, —CN, C₁₋₆ alkyl, C₃₋₆ cycloalkyl, or C₆₋₁₀ aryl. In some embodiments, each R^{g} is independently hydrogen, halogen, —CN, C₁₋₆ alkyl, C₃₋₆ cycloalkyl, C₆₋₁₀ aryl, or 5- to 10-membered heteroaryl having 1-5 heteroatoms independently selected from N, O, and S. In some embodiments, each R^{g} is independently hydrogen, halogen, —CN, C₁₋₆ alkyl, C₃₋₆ cycloalkyl, C₆₋₁₀ aryl, 5- to 10-membered heteroaryl having 1-5 heteroatoms independently selected from N, O, and S, or 3- to 10-membered monocyclic heterocyclyl having 1-5 heteroatoms independently selected from N, O, and S. In some embodiments, each R^{g} is independently hydrogen. In some embodiments. R^{g} is halogen. In some embodiments. R^{g} is fluoro. In some embodiments, R^{g} is chloro. In some embodiments, R^{g} is bromo. In some embodiments. R^{g} is —CN. In some embodiments, R^{g} is optionally substituted C₁₋₆ aliphatic. In some embodiments, R^{g} is optionally substituted C₁₋₆ alkyl. In some embodiments, R⁸ is C₁₋₂ alkyl (e.g., methyl). In some embodiments, R^{g} is optionally substituted C₃₋₆ cycloaliphatic. In some embodiments, R^{g} is optionally substituted C₃₋₆ cycloalkyl. In some embodiments, R^{g} is C₃₋₄ cycloalkyl (e.g., cyclopropyl). In some embodiments. R^{g} is C₆₋₁₀ aryl. In some embodiments. R^{g} is phenyl. In some embodiments. R^{g} is naphthyl. In some embodiments, R^{g} is 5- to 10-membered heteroaryl having 1-5 heteroatoms independently selected from N, O, and S. In some embodiments, R^{g} is 5- to 6-membered heteroaryl having 1-3 heteroatoms independently selected from N, O, and S. In some embodiments, R^{g} is 3- to 10-membered monocyclic heterocyclyl having 1-5 heteroatoms independently selected from N, O, and S. In some embodiments, R^{g} is 4- to 6-membered monocyclic heterocyclyl having 1-3 heteroatoms independently selected from N, O, and S.

In some embodiments, R^{h} is —R as described herein. In some embodiments, R^{h} is —R but is not hydrogen. In some embodiments of any Formulae described herein, R^{h} is hydrogen, halogen, or optionally substituted C₁₋₆ alkyl. In some embodiments, R^{h} is hydrogen. In some embodiments. R^{h} is halogen (e.g., fluoro or chloro). In some embodiments, R^{h} is optionally substituted C₁₋₆ aliphatic. In some embodiments, R^{h} is optionally substituted C₁₋₆ alkyl. In some embodiments, R^{h} is C₁₋₆ alkyl. In some embodiments, R^{h} is optionally substituted C₁₋₂ alkyl. In some embodiments, R^{h} is C₁₋₂ alkyl (e.g., methyl). In some embodiments of any Formulae described herein, R^{h} is hydrogen, halogen, or —R. In some embodiments, each R^{h} is independently hydrogen, halogen, or optionally substituted C₁₋₆ aliphatic. In some embodiments, each R^{h} is independently hydrogen, halogen, C₁₋₆ alkyl, or Cas cycloalkyl. In some embodiments, each R^{h} is independently is hydrogen, halogen, C₁₋₆ alkyl, C₃₋₆ cycloalkyl, or C₆₋₁₀ aryl. In some embodiments, each R^{h} is independently hydrogen, halogen, C₁₋₆ alkyl, C₃₋₆ cycloalkyl, C₆₋₁₀ aryl, or 5- to 10-membered heteroaryl having 1-5 heteroatoms independently selected from N, O, and S. In some embodiments, each R^{h} is independently hydrogen, halogen, C₁₋₆ alkyl, C₃-6 cycloalkyl, C₆₋₁₀ aryl, 5- to 10-membered heteroaryl having 1-5 heteroatoms independently selected from N, O, and S, or 3- to 10-membered monocyclic heterocyclyl having 1-5 heteroatoms independently selected from N, O, and S. In some embodiments, R^{h} is halogen. In some embodiments, R^{h} is fluoro. In some embodiments, R^{h} is chloro. In some embodiments, R^{h} is bromo. In some embodiments, R^{h} is optionally substituted C₁₋₆ aliphatic. In some embodiments, R^{h} is optionally substituted C₃₋₆ cycloaliphatic. In some embodiments, R^{h} is optionally substituted C₃₋₆ cycloalkyl. In some embodiments, R^{h} is C₃₋₄ cycloalkyl (e.g., cyclopropyl). In some embodiments, R^{h} is C₆₋₁₀ aryl. In some embodiments, R^{h} is phenyl. In some embodiments, R^{h} is naphthyl. In some embodiments, R^{h} is 5- to 10-membered heteroaryl having 1-5 heteroatoms independently selected from N, O, and S. In some embodiments, R^{h} is 5- to 6-membered heteroaryl having 1-3 heteroatoms independently selected from N, O, and S. In some embodiments, R^{h} is 3- to 10-membered monocyclic heterocyclyl having 1-5 heteroatoms independently selected from N, O, and S. In some embodiments, R^{h} is 4- to 6-membered monocyclic heterocyclyl having 1-3 heteroatoms independently selected from N, O, and S.

In some embodiments of any Formulae described herein, p is 0, 1, or 2. In some embodiments of any Formulae described herein, p is 0, 1, 2, or 3. In some embodiments, p is 0 or 1. In some embodiments, p is 1, 2, or 3. In some embodiments, p is 0. In some embodiments, p is 1. In some embodiments, p is 2. In some embodiments, p is 3.

In some embodiments of any Formulae described herein, a LBM is an Inhibitor of Apoptosis protein (IAP) binding moiety, i.e., is a moiety that is capable of binding Inhibitor of Apoptosis protein.

In some embodiments of any Formulae described herein, a LBM has the following structure:

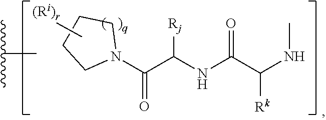

wherein Rⁱ, R^{j}, R^{k}, r, and q are as defined herein for Formula V and described in classes and subclasses herein, both singly and in combination.

In some embodiments of any Formulae described herein, a LBM is selected from:

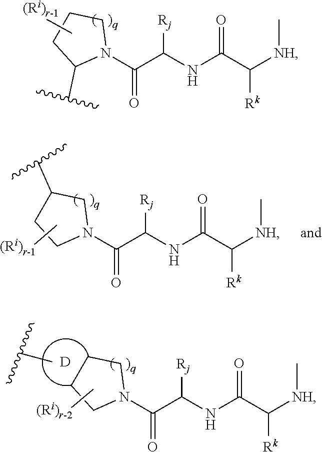

wherein Rⁱ, R^{j}, R^{k}, r, and q are as defined herein for Formula V and described in classes and subclasses herein, both singly and in combination; and Ring D is the ring formed when two Rⁱ groups, together with the atoms to which they are attached, combine.

In some embodiments of any Formulae described herein, a LBM has the following structure:

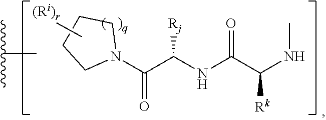

wherein Rⁱ, R^{j}, R^{k}, r, and q are as defined herein for Formula V and described in classes and subclasses herein, both singly and in combination.

In some embodiments of any Formulae described herein, a LBM is selected from:

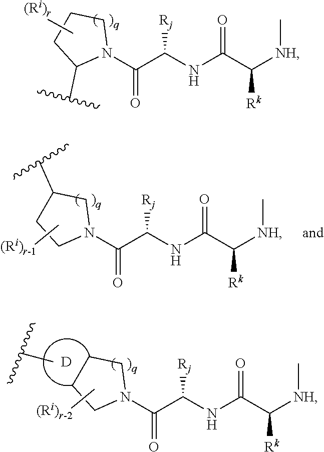

wherein Rⁱ, R^{j}, R^{k}, r, and q are as defined herein for Formula V and described in classes and subclasses herein, both singly and in combination; and Ring D is the ring formed when two Rⁱ groups, together with the atoms to which they are attached, combine.

In some embodiments of any Formulae described herein, a LBM has the following structure:

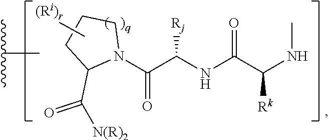

wherein R, Rⁱ, R^{j}, R^{k}, r, and q are as defined herein for Formula V and described in classes and subclasses herein, both singly and in combination.

In some embodiments of any Formulae described herein, a LBM has the following structure:

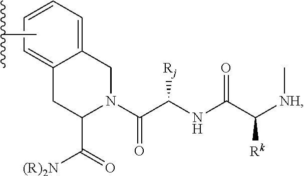

wherein R, R^{j}, and R^{k} are as defined herein for Formula V and described in classes and subclasses herein, both singly and in combination.

In some embodiments of any Formulae described herein, a LBM has the following structure:

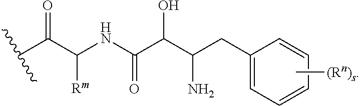

wherein R^{m}, Rⁿ, and s are as defined herein for Formula VI and described in classes and subclasses herein, both singly and in combination.

In some embodiments of any Formulae described herein, a LBM has the following structure:

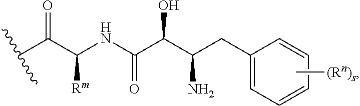

wherein R^{m}, Rⁿ, and s are as defined herein for Formula VI and described in classes and subclasses herein, both singly and in combination.

In some embodiments of any Formulae described herein, a LBM moiety (e.g., IAP binding moiety) is attached to linker at a Rⁱ. In some embodiments, a LBM moiety (e.g., IAP binding moiety) is attached to linker at the ring formed when two Rⁱ groups, together with the atoms to which they are attached, combine.

In some embodiments, Rⁱ is —R as described herein. In some embodiments, Rⁱ is —R but is not hydrogen. In some embodiments of any Formulae described herein, each R that is not the point of attachment of the linker is independently halogen, optionally substituted C₁₋₆ aliphatic, —C(O)N(R)₂, or —N(R)C(O)R. In some embodiments, each Rⁱ is —R. In some embodiments, Rⁱ is halogen (e.g., fluoro or chloro). In some embodiments, Rⁱ is optionally substituted C₁₋₆ aliphatic. In some embodiments, Rⁱ is optionally substituted C₁₋₆ alkyl. In some embodiments, Rⁱ is C₁₋₆ alkyl. In some embodiments, Rⁱ is optionally substituted C₁₋₂ alkyl. In some embodiments, Rⁱ is C₁₋₂ alkyl (e.g., methyl). In some embodiments, Rⁱ is —C(O)N(R)₂. In some embodiments, at least one Rⁱ is —C(O)N(R)₂. In some embodiments, Rⁱ is —N(R)C(O)R. In some embodiments, two Rⁱ groups, together with the atoms to which they are attached, combine to form an optionally substituted phenyl or 5- to 6-membered heteroaryl ring having 1-3 heteroatoms independently selected from N, O, and S. In some embodiments, two Rⁱ groups, together with the atoms to which they are attached, combine to form an optionally substituted phenyl. In some embodiments, two Rⁱ groups, together with the atoms to which they are attached, combine to form an optionally substituted 5- to 6-membered heteroaryl ring having 1-3 heteroatoms independently selected from N, O, and S.

In some embodiments, R^{j} is —R as described herein. In some embodiments, R^{j} is —R but is not hydrogen. In some embodiments of any Formulae described herein, R^{j} is an optionally substituted group selected from C₁₋₆ aliphatic and C₃₋₇ cycloaliphatic. In some embodiments, R^{j} is optionally substituted C₁₋₆ aliphatic. In some embodiments, R^{j} is optionally substituted C₁₋₆ alkyl. In some embodiments, R is C₁₋₆ alkyl (e.g., tert-butyl or isopropyl). In some embodiments, R^{j} is optionally substituted C₁₋₂ alkyl. In some embodiments, R^{j} is C₁₋₂ alkyl (e.g., methyl). In some embodiments, R^{j} is optionally substituted C₃₋₇ cycloaliphatic. In some embodiments, R^{j} is optionally substituted C₃₋₇ cycloalkyl. In some embodiments, R^{j} is C₃₋₇ cycloalkyl (e.g., cyclohexyl). In some embodiments, R^{j} combines with one instance of R^{j}, together with the atoms to which they are attached, to form an optionally substituted 5- to 7-membered heterocycle having 1-2 heteroatoms independently selected from N, O, and S. In some embodiments of any Formulae described herein, R^{j} is —R. In some embodiments, R^{j} is hydrogen, or optionally substituted C₁₋₆ aliphatic. In some embodiments, R^{j} is hydrogen, C₁₋₆ alkyl, or C₃₋₆ cycloalkyl. In some embodiments, R^{j} is hydrogen, C₁₋₆ alkyl, C₃₋₆ cycloalkyl, or C₆₋₁₀ aryl. In some embodiments, R is hydrogen, C₁₋₆ alkyl, C₃₋₆ cycloalkyl, C₆₋₁₀ aryl, or 5- to 10-membered heteroaryl having 1-5 heteroatoms independently selected from N, O, and S. In some embodiments, R^{j} is hydrogen, C₁₋₆ alkyl, C₃₋₆ cycloalkyl, C₆₋₁₀ aryl, 5- to 10-membered heteroaryl having 1-5 heteroatoms independently selected from N, O, and S, or 3- to 10-membered monocyclic heterocyclyl having 1-5 heteroatoms independently selected from N, O, and S. In some embodiments, R^{j} is hydrogen. In some embodiments, R^{j} is optionally substituted C₁₋₆ aliphatic. In some embodiments, R^{j} is optionally substituted C₁₋₆ alkyl. In some embodiments, R^{j} is C₁₋₂ alkyl (e.g., methyl). In some embodiments, R^{j} is optionally substituted C₃₋₆ cycloaliphatic. In some embodiments, R^{j} is optionally substituted C₃₋₆ cycloalkyl. In some embodiments, R^{j} is C₃₋₄ cycloalkyl (e.g., cyclopropyl). In some embodiments, R^{j} is C₆₋₁₀ aryl. In some embodiments, R^{j} is phenyl. In some embodiments, R^{j} is naphthyl. In some embodiments, R^{j} is 5- to 10-membered heteroaryl having 1-5 heteroatoms independently selected from N, O, and S. In some embodiments, R^{j} is 5- to 6-membered heteroaryl having 1-3 heteroatoms independently selected from N, O, and S. In some embodiments, R^{j} is 3- to 10-membered monocyclic heterocyclyl having 1-5 heteroatoms independently selected from N, O, and S. In some embodiments, R^{j} is 4- to 6-membered monocyclic heterocyclyl having 1-3 heteroatoms independently selected from N, O, and S.

In some embodiments, R^{k} is —R as described herein. In some embodiments, R^{k} is —R but is not hydrogen. In some embodiments of any Formulae described herein, R^{k} is an optionally substituted C₁₋₆ alkyl. In some embodiments, R^{k} is C₁₋₆ alkyl. In some embodiments, R^{k} is optionally substituted C₁₋₂ alkyl. In some embodiments, R^{k} is C₁₋₂ alkyl (e.g., methyl). In some embodiments of any Formulae described herein, R^{k} is —R. In some embodiments, R^{k} is hydrogen, or optionally substituted C₁₋₆ aliphatic. In some embodiments, R^{k} is hydrogen, C₁₋₆ alkyl, or C₃₋₆ cycloalkyl. In some embodiments, R^{k} is hydrogen, C₁₋₆ alkyl, C₃₋₆ cycloalkyl, or C₆₋₁₀ aryl. In some embodiments, R^{k} is hydrogen, C₁₋₆ alkyl, C₃₋₆ cycloalkyl, C₆₋₁₀ aryl, or 5- to 10-membered heteroaryl having 1-5 heteroatoms independently selected from N, O, and S. In some embodiments, R^{k} is hydrogen, C₁₋₆ alkyl, C₃₋₆ cycloalkyl, C₆₋₁₀ aryl, 5- to 10-membered heteroaryl having 1-5 heteroatoms independently selected from N, O, and S, or 3- to 10-membered monocyclic heterocyclyl having 1-5 heteroatoms independently selected from N, O, and S. In some embodiments, R^{k} is hydrogen. In some embodiments, R^{k} is optionally substituted C₁₋₆ aliphatic. In some embodiments, R^{k} is optionally substituted C₁₋₆ alkyl. In some embodiments, R^{k} is C₁₋₂ alkyl (e.g., methyl). In some embodiments, R^{k} is optionally substituted C₃₋₆ cycloaliphatic. In some embodiments, R^{k} is optionally substituted C₃₋₆ cycloalkyl. In some embodiments, R^{k} is C₃₋₄ cycloalkyl (e.g., cyclopropyl). In some embodiments, R^{k} is C₆₋₁₀ aryl. In some embodiments, R^{k} is phenyl. In some embodiments, R^{k} is naphthyl. In some embodiments, R^{k} is 5- to 10-membered heteroaryl having 1-5 heteroatoms independently selected from N, O, and S. In some embodiments, R^{k} is 5- to 6-membered heteroaryl having 1-3 heteroatoms independently selected from N, O, and S. In some embodiments. R^{k} is 3- to 10-membered monocyclic heterocyclyl having 1-5 heteroatoms independently selected from N, O, and S. In some embodiments, R^{k} is 4- to 6-membered monocyclic heterocyclyl having 1-3 heteroatoms independently selected from N, O, and S.

In some embodiments of any Formulae described herein, r is 1, 2, or 3. In some embodiments, r is 1 or 2. In some embodiments, r is 1, 2, 3, 4, or 5. In some embodiments, r is 0 or 1. In some embodiments, r is 1, 2, or 3. In some embodiments, r is 0. In some embodiments, r is 1. In some embodiments, r is 2. In some embodiments, r is 3. In some embodiments, r is 4. In some embodiments, r is 5.

In some embodiments of any Formulae described herein, q is 1. In some embodiments, q is 2.

In some embodiments, R^{m} is —R as described herein. In some embodiments, R^{m} is —R but is not hydrogen. In some embodiments of any Formulae described herein, R^{m} is optionally substituted C₁₋₆ alkyl. In some embodiments, R^{m} is C₁₋₆ alkyl (e.g., isobutyl). In some embodiments, R^{m} is optionally substituted C₁₋₂ alkyl. In some embodiments, R^{m} is C₁₋₂ alkyl (e.g., methyl). In some embodiments of any Formulae described herein, R^{m} is —R. In some embodiments, R^{m} is hydrogen, or optionally substituted C₁₋₆ aliphatic. In some embodiments, R^{m} is hydrogen, C₁₋₆ alkyl, or C₃₋₆ cycloalkyl. In some embodiments, R^{m} is hydrogen, C₁₋₆ alkyl, C₃₋₆ cycloalkyl, or C₆₋₁₀ aryl. In some embodiments, R^{m} is hydrogen, C₁₋₆ alkyl, C₃₋₆ cycloalkyl, C₆₋₁₀ aryl, or 5- to 10-membered heteroaryl having 1-5 heteroatoms independently selected from N, O, and S. In some embodiments, R^{m} is hydrogen, C₁₋₆ alkyl, C₃₋₆ cycloalkyl, C₆₋₁₀ aryl, 5- to 10-membered heteroaryl having 1-5 heteroatoms independently selected from N, O, and S, or 3- to 10-membered monocyclic heterocyclyl having 1-5 heteroatoms independently selected from N, O, and S. In some embodiments, R^{m} is hydrogen. In some embodiments, R^{m} is optionally substituted C₁₋₆ aliphatic. In some embodiments, R^{m} is optionally substituted C₃₋₆ cycloaliphatic. In some embodiments, R^{m} is optionally substituted C₃₋₆ cycloalkyl. In some embodiments, R^{m} is C₃₋₄ cycloalkyl (e.g., cyclopropyl). In some embodiments. R^{m} is C₆₋₁₀ aryl. In some embodiments, R^{m} is phenyl. In some embodiments, R^{m} is naphthyl. In some embodiments, R^{m} is 5- to 10-membered heteroaryl having 1-5 heteroatoms independently selected from N, O, and S. In some embodiments, R^{m} is 5- to 6-membered heteroaryl having 1-3 heteroatoms independently selected from N, O, and S. In some embodiments, R^{m} is 3- to 10-membered monocyclic heterocyclyl having 1-5 heteroatoms independently selected from N, O, and S. In some embodiments, R^{m} is 4- to 6-membered monocyclic heterocyclyl having 1-3 heteroatoms independently selected from N, O, and S.

In some embodiments, Rⁿ is —R as described herein. In some embodiments, Rⁿ is —R but is not hydrogen. In some embodiments. Rⁿ is —OR wherein R is as described herein. In some embodiments of any Formulae described herein, each Rⁿ is independently halogen, —OR, —CN, or optionally substituted C₁₋₆ alkyl. In some embodiments, Rⁿ is —OR (e.g., —OH or —O(C₁₋₆ alkyl)). In some embodiments, Rⁿ is halogen (e.g., fluoro or chloro). In some embodiments, Rⁿ is —OR. In some embodiments, Rⁿ is —CN. In some embodiments, Rⁿ is optionally substituted C₁₋₆ aliphatic. In some embodiments, Rⁿ is optionally substituted C₁₋₆ alkyl. In some embodiments, Rⁿ is C₁₋₆ alkyl. In some embodiments, Rⁿ is optionally substituted C₁₋₂ alkyl. In some embodiments, Rⁿ is C₁₋₂ alkyl (e.g., methyl).

In some embodiments of any Formulae described herein, each of Rⁿ, R^{p}, R^{q}, R^{r}, R^{s}, and R^{u} is independently halogen, —CN, —R or —OR. In some embodiments, Rⁿ is —CN. In some embodiments, Rⁿ is —R. In some embodiments, Rⁿ is —OR. In some embodiments, R^{p} is —CN. In some embodiments, R^{p} is —R. In some embodiments, R^{p} is —OR. In some embodiments, R^{q} is —CN. In some embodiments, R^{q} is —R. In some embodiments, R^{q} is —OR. In some embodiments. R^{r} is —CN. In some embodiments, R^{r} is —R. In some embodiments, R^{r} is —OR. In some embodiments, R^{s} is —CN. In some embodiments. R^{s} is —R. In some embodiments, R^{s} is —OR. In some embodiments, R^{u} is —CN. In some embodiments, R^{u} is —R. In some embodiments, R^{u} is —OR.

In some embodiments of any Formulae described herein, s is 0, 1, or 2. In some embodiments, s is 1, 2, or 3. In some embodiments, s is 0, 1, 2, 3, 4, or 5. In some embodiments, s is 0 or 1. In some embodiments, s is 1, 2, or 3. In some embodiments, s is 0. In some embodiments, s is 1. In some embodiments, s is 2. In some embodiments, s is 3. In some embodiments, s is 4. In some embodiments, s is 5.

In some embodiments of any Formulae described herein, a LBM is a MDM2 binding moiety, i.e., is a moiety that is capable of binding MDM2.

In some embodiments of any Formulae described herein, a LBM has the following structure:

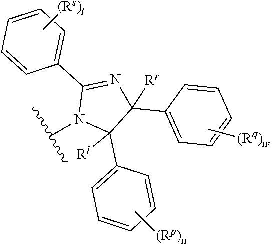

wherein R^{p}, R^{q}, R^{r}, R^{s}, u and t are as defined herein for Formula VII and described in classes and subclasses herein, both singly and in combination.

In some embodiments of any Formulae described herein, a LBM has the following structure:

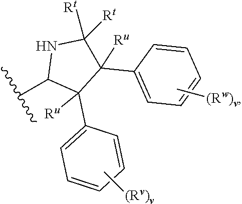

wherein R^{t}, R^{u}, R^{v}, R^{w}, and v are as defined herein for Formula VIII and described in classes and subclasses herein, both singly and in combination.

In some embodiments, R^{p} is —R as described herein. In some embodiments, R^{p} is hydrogen. In some embodiments, R^{p} is —R but is not hydrogen. In some embodiments, R^{p} is —OR wherein R is as described herein. In some embodiments of any Formulae described herein, each R^{p} is independently halogen, —OR, —CN, or optionally substituted C₁₋₆ alkyl. In some embodiments, R^{p} is —OR (e.g., —OH or —O(C₁₋₆ alkyl)). In some embodiments, each R^{p} is halogen (e.g., chloro). In some embodiments. R^{p} is halogen (e.g., chloro or fluoro). In some embodiments, R^{p} is —OR. In some embodiments, R^{p} is —CN. In some embodiments, R^{p} is optionally substituted C₁₋₆ aliphatic. In some embodiments, R^{p} is optionally substituted C₁₋₆ alkyl. In some embodiments, R^{p} is C₁₋₆ alkyl. In some embodiments, R^{p} is optionally substituted C₁₋₂ alkyl. In some embodiments, R^{p} is C₁₋₂ alkyl (e.g., methyl).

In some embodiments, R^{q} is —R as described herein. In some embodiments, R^{q} is hydrogen. In some embodiments, R^{q} is —R but is not hydrogen. In some embodiments, R^{q} is —OR wherein R is as described herein. In some embodiments of any Formulae described herein, each R^{q} is independently halogen, —OR, —CN, or optionally substituted C₁₋₆ alkyl. In some embodiments, each R^{q} is —OR (e.g., —OH or —O(C₁₋₆ alkyl)). In some embodiments, each R^{q} is halogen (e.g., chloro). In some embodiments, R^{q} is halogen (e.g., chloro or fluoro). In some embodiments, R^{q} is —OR. In some embodiments, R^{q} is —CN. In some embodiments, Ry is optionally substituted C₁₋₆ aliphatic. In some embodiments, R^{q} is optionally substituted C₁₋₆ alkyl. In some embodiments, R^{q} is C₁₋₆ alkyl. In some embodiments, R^{q} is optionally substituted C₁₋₂ alkyl. In some embodiments, R^{q} is C₁₋₂ alkyl (e.g., methyl).

In some embodiments, R^{r} is —R as described herein. In some embodiments, R^{r} is —R but is not hydrogen. In some embodiments. R^{r} is —OR wherein R is as described herein. In some embodiments, R^{r} is halogen (e.g., —F, —Cl or —Br). In some embodiments, R^{r} is —CN. In some embodiments of any Formulae described herein, each R^{r} is independently hydrogen or optionally substituted C₁₋₆ alkyl. In some embodiments, R^{r} is hydrogen. In some embodiments, R^{r} is optionally substituted C₁₋₆ aliphatic. In some embodiments, R^{r} is optionally substituted C₁₋₆ alkyl. In some embodiments, R^{r} is C₁₋₆ alkyl. In some embodiments, R^{r} is optionally substituted C₁₋₂ alkyl. In some embodiments, R^{r} is C₁₋₂ alkyl (e.g., methyl). In some embodiments, each R^{r} is methyl.

In some embodiments, R^{s} is —R as described herein. In some embodiments, R^{s} is hydrogen. In some embodiments, R^{s} is —R but is not hydrogen. In some embodiments, R^{s} is —OR wherein R is as described herein. In some embodiments, R^{s} is halogen (e.g., —F, —Cl or —Br). In some embodiments of any Formulae described herein, each R^{s} is independently halogen, —OR, —CN, or optionally substituted C₁₋₆ alkyl. In some embodiments, each R^{s} is halogen (e.g., chloro). In some embodiments, R^{s} is halogen (e.g., chloro or fluoro). In some embodiments. R^{s} is —OR (e.g., —OH or —O(C₁₋₆ alkyl)). In some embodiments, R^{s} is —OR (e.g., —O(C₁₋₆ alkyl)). In some embodiments, R^{s} is —CN. In some embodiments, R^{s} is optionally substituted C₁₋₆ aliphatic. In some embodiments, R^{s} is optionally substituted C₁₋₆ alkyl. In some embodiments, R^{s} is C₁₋₆ alkyl (e.g., tert-butyl). In some embodiments, R^{s} is optionally substituted C₁₋₂ alkyl. In some embodiments, R^{s} is C₁₋₂ alkyl (e.g., methyl).

In some embodiments of any Formulae described herein, t is 0, 1, or 2. In some embodiments, t is 0, 1, 2, 3, 4, or 5. In some embodiments, t is 0, 1, 2, 3, or 4. In some embodiments, t is 0 or 1. In some embodiments, t is 1, 2, or 3. In some embodiments, t is 0. In some embodiments, t is 1. In some embodiments, t is 2. In some embodiments, t is 3. In some embodiments, t is 4.

In some embodiments of any Formulae described herein, each u is independently 0, 1, or 2. In some embodiments, each u is independently 1, 2, or 3. In some embodiments, each u is independently 0 or 1. In some embodiments, each u is independently 0, 1, 2, 3, 4, or 5. In some embodiments, u is 0 or 1. In some embodiments, u is 1, 2, or 3. In some embodiments, u is 0. In some embodiments, u is 1. In some embodiments, u is 2. In some embodiments, u is 3. In some embodiments, u is 4. In some embodiments, u is 5.

In some embodiments, R^{t} is —R as described herein. In some embodiments, R^{t} is —R but not hydrogen. In some embodiments of any Formulae described herein, each R^{t} is independently hydrogen or optionally substituted C₁₋₆ aliphatic. In some embodiments, R^{t} is hydrogen. In some embodiments, R^{t} is optionally substituted C₁₋₆ aliphatic. In some embodiments, R^{t} is optionally substituted C₁₋₆ alkyl. In some embodiments, R^{t} is C₁₋₆ alkyl (e.g., tert-butyl or isobutyl). In some embodiments, R^{t} is optionally substituted C₁₋₂ alkyl. In some embodiments, R^{t} is C₁₋₂ alkyl (e.g., methyl). In some embodiments, at least one R^{t} is optionally substituted C₁₋₆ aliphatic. In some embodiments of any Formulae described herein, each R^{t} is independently —R. In some embodiments, R^{t} is hydrogen, halogen, or optionally substituted C₁₋₆ aliphatic. In some embodiments, R^{t} is hydrogen, halogen, —CN, C₁₋₆ alkyl, or C₃₋₆ cycloalkyl. In some embodiments, R^{t} is hydrogen, halogen, —CN, C₁₋₆ alkyl, C₃₋₆ cycloalkyl, or C₆₋₁₀ aryl. In some embodiments, R^{t} is hydrogen, halogen, —CN, C₁₋₆ alkyl, C₃₋₆ cycloalkyl, C₆₋₁₀ aryl, or 5- to 10-membered heteroaryl having 1-5 heteroatoms independently selected from N, O, and S. In some embodiments, R^{t} is hydrogen, halogen, —CN, C₁₋₆ alkyl, C₃₋₆ cycloalkyl, C₅₋₁₀ aryl, 5- to 10-membered heteroaryl having 1-5 heteroatoms independently selected from N, O, and S, or 3- to 10-membered monocyclic heterocyclyl having 1-5 heteroatoms independently selected from N, O, and S. In some embodiments, R^{t} is hydrogen. In some embodiments, R^{t} is halogen. In some embodiments, R^{t} is fluoro. In some embodiments. R^{t} is chloro. In some embodiments, R^{t} is bromo. In some embodiments, R^{t} is optionally substituted C₃₋₆ cycloaliphatic. In some embodiments, R^{t} is optionally substituted C₃₋₆ cycloalkyl. In some embodiments, R^{t} is C₃₋₄ cycloalkyl (e.g., cyclopropyl). In some embodiments, R^{t} is C₆₋₁₀ aryl. In some embodiments. R^{t} is phenyl. In some embodiments, R^{t} is naphthyl. In some embodiments, R^{t} is 5- to 10-membered heteroaryl having 1-5 heteroatoms independently selected from N, O, and S. In some embodiments, R^{t} is 5- to 6-membered heteroaryl having 1-3 heteroatoms independently selected from N, O, and S. In some embodiments, R^{t} is 3- to 10-membered monocyclic heterocyclyl having 1-5 heteroatoms independently selected from N, O, and S. In some embodiments, R^{t} is 4- to 6-membered monocyclic heterocyclyl having 1-3 heteroatoms independently selected from N, O, and S.

In some embodiments, both R^{t} groups, together with the atom to which they are attached, combine to form an optionally substituted 3- to 7-membered ring having 0-2 heteroatoms independently selected from N, O, and S. In some embodiments, both R^{t} groups, together with the atom to which they are attached, combine to form an optionally substituted 3- to 7-membered cycloaliphatic or heterocycle having 1-2 heteroatoms independently selected from N, O, and S. In some embodiments, both R^{t} groups, together with the atom to which they are attached, combine to form an optionally substituted C₃₋₇ cycloaliphatic (e.g., C₃₋₇ cycloalkyl, such as cyclohexyl). In some embodiments, both R^{t} groups, together with the atom to which they are attached, combine to form an optionally substituted 3- to 7-membered heterocycle having 1-2 heteroatoms independently selected from N, O, and S.

In some embodiments, R^{u} is —R as described herein. In some embodiments, R^{u} is —R but not hydrogen. In some embodiments, R^{u} is —OR wherein R is as described herein. In some embodiments of any Formulae described herein, each R^{u} is independently hydrogen, halogen, —CN, or optionally substituted C₁₋₆ alkyl. In some embodiments, R^{u} is hydrogen. In some embodiments, at least one R^{u} is hydrogen. In some embodiments, R^{u} is halogen (e.g., fluoro or chloro). In some embodiments, R^{u} is —CN. In some embodiments, R^{u} is optionally substituted C₁₋₆ aliphatic. In some embodiments, R^{u} is optionally substituted C₁₋₆ alkyl. In some embodiments, R^{u} is C₁₋₆ alkyl. In some embodiments, R^{u} is optionally substituted C₁₋₂ alkyl. In some embodiments, R^{u} is C₁₋₂ alkyl (e.g., methyl).

In some embodiments, R^{v} is —R as described herein. In some embodiments, R^{v} is —R but not hydrogen. In some embodiments, R^{v} is —OR wherein R is as described herein. In some embodiments of any Formulae described herein, each R^{v} is independently halogen, —OR, —CN, or optionally substituted C₁₋₆ alkyl. In some embodiments, each R^{v} is halogen (e.g., chloro). In some embodiments, R^{v} is halogen (e.g., chloro or fluoro). In some embodiments, R^{v} is —OR (e.g., —OH or —O(C₁₋₆ alkyl)). In some embodiments, R^{v} is —OR (e.g., —O(C₁₋₆ alkyl)). In some embodiments, R^{v} is —CN. In some embodiments, R^{v} is optionally substituted C₁₋₆ aliphatic. In some embodiments. R^{v} is optionally substituted C₁₋₆ alkyl. In some embodiments, R^{v} is C₁₋₆ alkyl (e.g., tert-butyl). In some embodiments, R^{v} is optionally substituted C₁₋₆ alkyl. In some embodiments, R^{v} is C₁₋₆ alkyl (e.g., methyl). In some embodiments of any Formulae described herein, each R^{v} is independently halogen, —CN, —R or —OR. In some embodiments, R^{v} is —OR. In some embodiments, R^{v} is hydrogen, halogen, or optionally substituted C₁₋₆ aliphatic. In some embodiments, R^{v} is hydrogen, halogen, —CN, C₁₋₆ alkyl, or C₃₋₆ cycloalkyl. In some embodiments, R^{v} is hydrogen, halogen, —CN, C₁₋₆ alkyl, C₃₋₆ cycloalkyl, or C₆₋₁₀ aryl. In some embodiments, R^{v} is hydrogen, halogen, —CN, C₁₋₆ alkyl, C₃-6 cycloalkyl, C₆₋₁₀ aryl, or 5- to 10-membered heteroaryl having 1-5 heteroatoms independently selected from N, O, and S. In some embodiments, R^{v} is hydrogen, halogen, —CN, C₁₋₆ alkyl, C₃₋₆ cycloalkyl, C₆₋₁₀ aryl, 5- to 10-membered heteroaryl having 1-5 heteroatoms independently selected from N, O, and S, or 3- to 10-membered monocyclic heterocyclyl having 1-5 heteroatoms independently selected from N, O, and S. In some embodiments, R^{v} is hydrogen. In some embodiments, R^{v} is halogen. In some embodiments, R^{v} is fluoro. In some embodiments, R^{v} is chloro. In some embodiments, R^{v} is bromo. In some embodiments. R^{v} is optionally substituted C₃₋₆ cycloaliphatic. In some embodiments, R^{v} is optionally substituted C₃₋₆ cycloalkyl. In some embodiments, R^{v} is C₃₋₄ cycloalkyl (e.g., cyclopropyl). In some embodiments, R^{v} is C₆₋₁₀ aryl. In some embodiments, R^{v} is phenyl. In some embodiments, R^{v} is naphthyl. In some embodiments, R^{v} is 5- to 10-membered heteroaryl having 1-5 heteroatoms independently selected from N, O, and S. In some embodiments, R^{v} is 5- to 6-membered heteroaryl having 1-3 heteroatoms independently selected from N, O, and S. In some embodiments, R^{v} is 3- to 10-membered monocyclic heterocyclyl having 1-5 heteroatoms independently selected from N, O, and S. In some embodiments, R^{v} is 4- to 6-membered monocyclic heterocyclyl having 1-3 heteroatoms independently selected from N, O, and S.

In some embodiments, one instance of R^{u} and R^{w}, together with the atoms to which they are attached, combine to form an optionally substituted 3- to 7-membered ring having 0-2 heteroatoms independently selected from N, O, and S. In some embodiments, one instance of R^{u} and R^{v}, together with the atoms to which they are attached, combine to form an optionally substituted 3- to 7-membered cycloaliphatic or heterocycle having 1-2 heteroatoms independently selected from N, O, and S. In some embodiments, one instance of R^{u} and R^{v}, together with the atoms to which they are attached, combine to form an optionally substituted 3- to 7-membered cycloaliphatic. In some embodiments, one instance of R^{u} and R^{v}, together with the atoms to which they are attached, combine to form an optionally substituted 3- to 7-membered heterocycle having 1-2 heteroatoms independently selected from N, O, and S.

In some embodiments, R^{w} is —R as described herein. In some embodiments. R^{w} is —R but not hydrogen. In some embodiments, R^{w} is —OR wherein R is as described herein. In some embodiments of any Formulae described herein, each R^{w} is independently halogen, —OR, —CN, or optionally substituted C₁₋₆ alkyl. In some embodiments, each R^{w} is halogen (e.g., chloro). In some embodiments, R^{w} is halogen (e.g., chloro or fluoro). In some embodiments, R^{w} is —OR (e.g., —O(C₁₋₆ alkyl)). In some embodiments, R^{w} is —CN. In some embodiments, R^{w} is optionally substituted C₁₋₆ aliphatic. In some embodiments, R^{w} is optionally substituted C₁₋₆ alkyl. In some embodiments, R^{w} is C₁₋₆ alkyl (e.g., tert-butyl). In some embodiments, R^{w} is optionally substituted C₁₋₂ alkyl. In some embodiments, R^{w} is C₁₋₂ alkyl (e.g., methyl). In some embodiments of any Formulae described herein, each R^{w} is independently halogen, —CN, —R or —OR. In some embodiments, R^{w} is —OR. In some embodiments, R^{w} is hydrogen, halogen, or optionally substituted C₁₋₆ aliphatic. In some embodiments, R^{w} is hydrogen, halogen, —CN, C₁₋₆ alkyl, or C₃₋₆ cycloalkyl. In some embodiments, R^{w} is hydrogen, halogen, —CN, C₁₋₆ alkyl, C₃₋₆ cycloalkyl, or C₆₋₁₀ aryl. In some embodiments, R^{w} is hydrogen, halogen, —CN, C₁₋₆ alkyl, C₃₋₆ cycloalkyl, C₆₋₁₀ aryl, or 5- to 10-membered heteroaryl having 1-5 heteroatoms independently selected from N, O, and S. In some embodiments, R^{w} is hydrogen, halogen, —CN, C₁₋₆ alkyl, C₃₋₆ cycloalkyl, C₆₋₁₀ aryl, 5- to 10-membered heteroaryl having 1-5 heteroatoms independently selected from N, O, and S, or 3- to 10-membered monocyclic heterocyclyl having 1-5 heteroatoms independently selected from N, O, and S. In some embodiments, R^{w} is hydrogen. In some embodiments, R^{w} is halogen. In some embodiments, R^{w} is fluoro. In some embodiments, R^{w} is chloro. In some embodiments, R^{w} is bromo. In some embodiments, R^{w} is optionally substituted C₁₋₆ aliphatic. In some embodiments, R^{w} is optionally substituted C₃₋₆ cycloaliphatic. In some embodiments, R^{w} is optionally substituted C₃₋₆ cycloalkyl. In some embodiments, R^{w} is C₃₋₄ cycloalkyl (e.g., cyclopropyl). In some embodiments. R^{w} is C₆₋₁₀ aryl. In some embodiments, R^{w} is phenyl. In some embodiments, R^{w} is naphthyl. In some embodiments, R^{w} is 5- to 10-membered heteroaryl having 1-5 heteroatoms independently selected from N, O, and S. In some embodiments, R^{w} is 5- to 6-membered heteroaryl having 1-3 heteroatoms independently selected from N, O, and S. In some embodiments, R^{w} is 3- to 10-membered monocyclic heterocyclyl having 1-5 heteroatoms independently selected from N, O, and S. In some embodiments, R^{v} is 4- to 6-membered monocyclic heterocyclyl having 1-3 heteroatoms independently selected from N, O, and S.

In some embodiments, one instance of R^{u} and R^{w}, together with the atoms to which they are attached, combine to form an optionally substituted 3- to 7-membered ring having 0-2 heteroatoms independently selected from N, O, and S. In some embodiments, one instance of R^{u} and R^{w}, together with the atoms to which they are attached, combine to form an optionally substituted 3- to 7-membered cycloaliphatic or heterocycle having 1-2 heteroatoms independently selected from N, O, and S. In some embodiments, one instance of R^{u} and R^{w}, together with the atoms to which they are attached, combine to form an optionally substituted 3- to 7-membered cycloaliphatic. In some embodiments, one instance of R^{u} and R^{w}, together with the atoms to which they are attached, combine to form an optionally substituted 3- to 7-membered heterocycle having 1-2 heteroatoms independently selected from N, O, and S.

In some embodiments of any Formulae described herein, each v is independently 0, 1, or 2. In some embodiments, each v is independently 1, 2, or 3. In some embodiments, each v is independently 0, 1, 2, 3, 4, or 5. In some embodiments, v is 0 or 1. In some embodiments, v is 1, 2, or 3. In some embodiments, v is 0. In some embodiments, v is 1. In some embodiments, v is 2. In some embodiments, v is 3. In some embodiments, v is 4. In some embodiments, v is 5.

In some embodiments, each R′ is independently —R, —C(O)R, or —S(O)₂R, or two R′ attached to the same atom, together with the atom to which they are attached, combine to form an optionally substituted 3- to 16-membered ring having 1-5 heteroatoms independently selected from N, O, and S. In some embodiments, each R′ is independently —R. In some embodiments, each R′ is independently —C(O)R. In some embodiments, each R′ is independently —S(O)₂R. In some embodiments, each R′ is independently two R′ attached to the same atom, together with the atom to which they are attached, combined to form an optionally substituted 3-16 membered ring having 1-5 heteroatoms independently selected from N, O, and S.

In some embodiments, each R is independently hydrogen or an optionally substituted group selected from C₁₋₈ aliphatic, C₃₋₁₀ cycloaliphatic, C₁-C₈ heteroaliphatic having 1-3 heteroatoms independently selected from N, O, and S, C₆₋₁₀ aryl, 5- to 10-membered heteroaryl having 1-5 heteroatoms independently selected from N, O, and S, and 3- to 10-membered monocyclic heterocyclyl having 1-5 heteroatoms independently selected from N, O, and S. In some embodiments, each R is independently hydrogen. In some embodiments, R is C₁₋₈ aliphatic. In some embodiments, R is C₆₋₁₀ aryl. In some embodiments, R is phenyl. In some embodiments, R is naphthyl. In some embodiments, R is C₃₋₁₀ cycloaliphatic. In some embodiments, R is 5- to 10-membered heteroaryl having 1-5 heteroatoms independently selected from N, O, and S. In some embodiments, R is 3- to 10-membered monocyclic heterocyclyl having 1-5 heteroatoms independently selected from N, O, and S. In some embodiments, each R is independently hydrogen or an optionally substituted group selected from C₁₋₆ aliphatic, phenyl, C₃₋₇ cycloaliphatic, 5- to 6-membered monocyclic heteroaryl having 1-4 heteroatoms independently selected from N, O, and S, and a 3- to 7-membered monocyclic heterocyclyl having 1-2 heteroatoms independently selected from N, O, and S.

In some embodiments of any Formulae described herein, the compound is not:

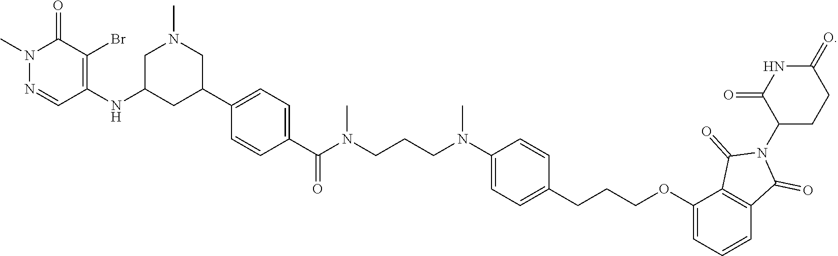 [SMILES: CN1CC(Nc2cnn(C)c(=O)c2Br)CC(c2ccc(C(=O)N(C)CCCN(C)c3ccc(CCCOc4cccc5c4C(=O)N(C4CCC(=O)NC4=O)C5=O)cc3)cc2)C1]

In some embodiments, the present disclosure provides compounds selected from Table 1, or a pharmaceutically acceptable salt thereof.

TABLE 1

| Compound No. | Structure |
|---|---|
| 1 | 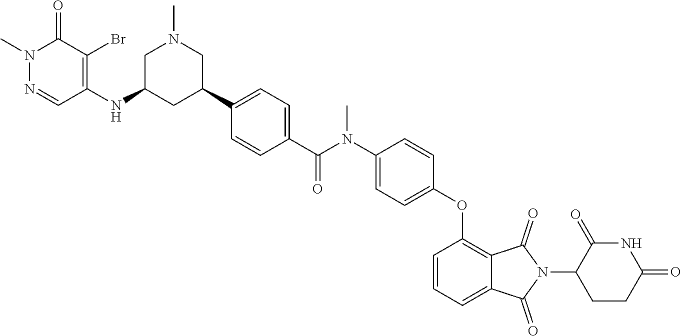 [SMILES: CN1C[C@H](Nc2cnn(C)c(=O)c2Br)C[C@H](c2ccc(C(=O)N(C)c3ccc(Oc4cccc5c4C(=O)N(C4CCC(=O)NC4=O)C5=O)cc3)cc2)C1] |
| 1a | 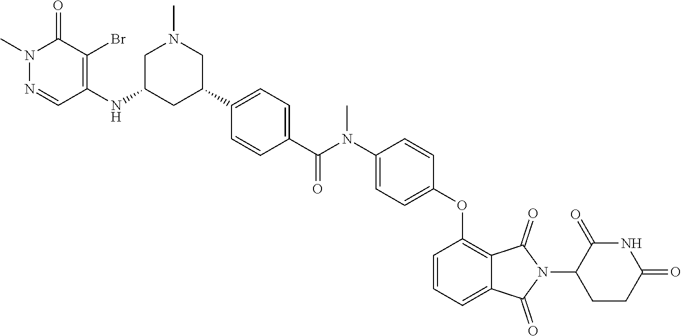 [SMILES: CN1C[C@@H](Nc2cnn(C)c(=O)c2Br)C[C@@H](c2ccc(C(=O)N(C)c3ccc(Oc4cccc5c4C(=O)N(C4CCC(=O)NC4=O)C5=O)cc3)cc2)C1] |
| 1b | 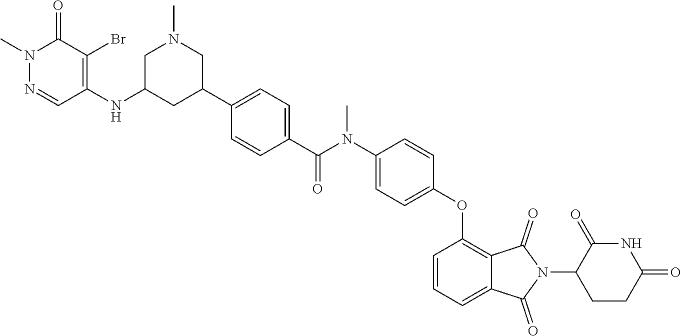 [SMILES: CN1CC(Nc2cnn(C)c(=O)c2Br)CC(c2ccc(C(=O)N(C)c3ccc(Oc4cccc5c4C(=O)N(C4CCC(=O)NC4=O)C5=O)cc3)cc2)C1] |
| 2 | 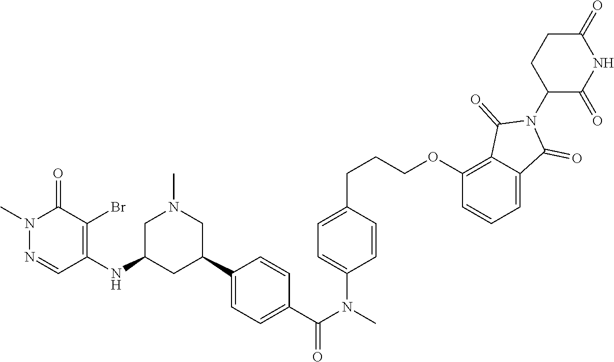 [SMILES: CN1C[C@H](Nc2cnn(C)c(=O)c2Br)C[C@H](c2ccc(C(=O)N(C)c3ccc(CCCOc4cccc5c4C(=O)N(C4CCC(=O)NC4=O)C5=O)cc3)cc2)C1] |
| 2a | 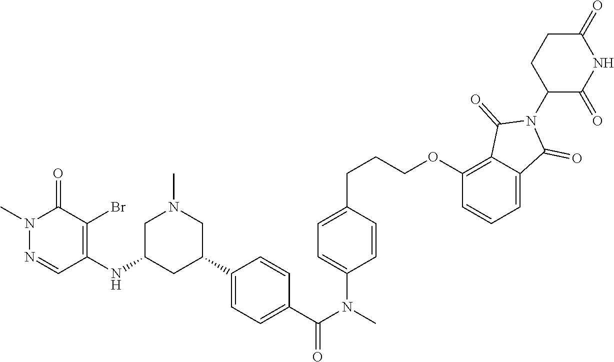 [SMILES: CN1C[C@@H](Nc2cnn(C)c(=O)c2Br)C[C@@H](c2ccc(C(=O)N(C)c3ccc(CCCOc4cccc5c4C(=O)N(C4CCC(=O)NC4=O)C5=O)cc3)cc2)C1] |
| 2b | 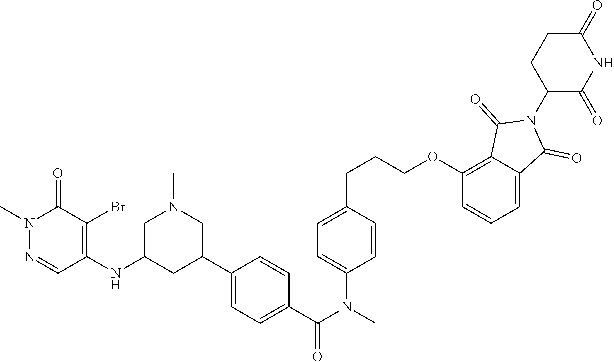 [SMILES: CN1CC(Nc2cnn(C)c(=O)c2Br)CC(c2ccc(C(=O)N(C)c3ccc(CCCOc4cccc5c4C(=O)N(C4CCC(=O)NC4=O)C5=O)cc3)cc2)C1] |
| 3 | 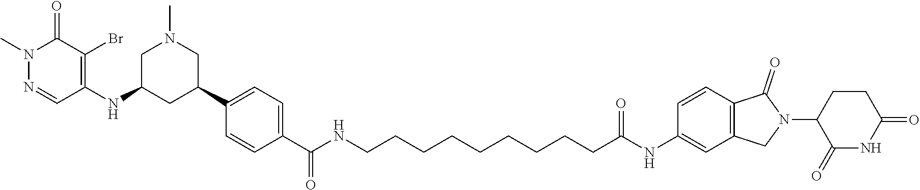 [SMILES: CN1C[C@H](Nc2cnn(C)c(=O)c2Br)C[C@H](c2ccc(C(=O)NCCCCCCCCCC(=O)Nc3ccc4c(c3)CN(C3CCC(=O)NC3=O)C4=O)cc2)C1] |
| 3a | 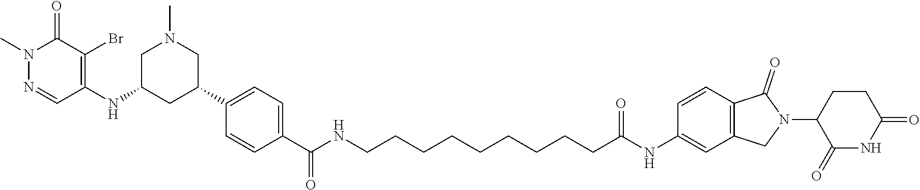 [SMILES: CN1C[C@@H](Nc2cnn(C)c(=O)c2Br)C[C@@H](c2ccc(C(=O)NCCCCCCCCCC(=O)Nc3ccc4c(c3)CN(C3CCC(=O)NC3=O)C4=O)cc2)C1] |
| 3b | 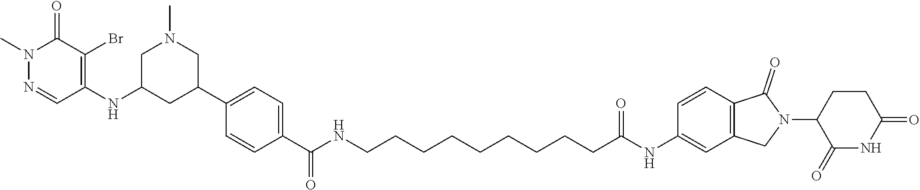 [SMILES: CN1CC(Nc2cnn(C)c(=O)c2Br)CC(c2ccc(C(=O)NCCCCCCCCCC(=O)Nc3ccc4c(c3)CN(C3CCC(=O)NC3=O)C4=O)cc2)C1] |
| 4 | 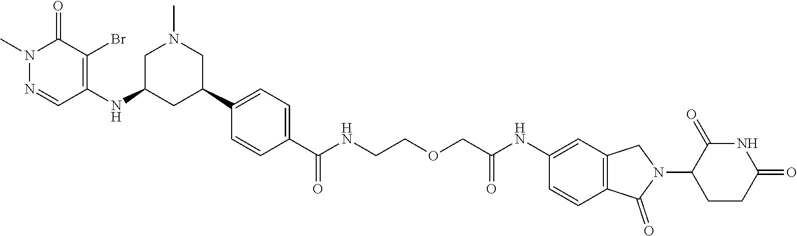 [SMILES: CN1C[C@H](Nc2cnn(C)c(=O)c2Br)C[C@H](c2ccc(C(=O)NCCOCC(=O)Nc3ccc4c(c3)CN(C3CCC(=O)NC3=O)C4=O)cc2)C1] |
| 4a | 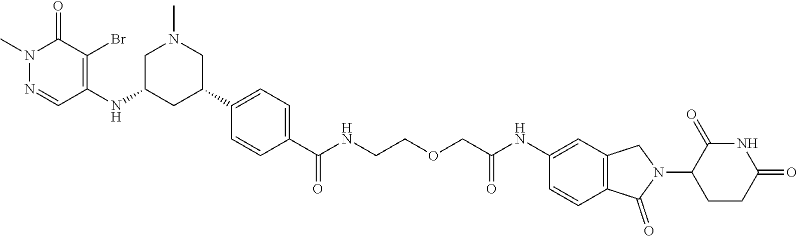 [SMILES: CN1C[C@@H](Nc2cnn(C)c(=O)c2Br)C[C@@H](c2ccc(C(=O)NCCOCC(=O)Nc3ccc4c(c3)CN(C3CCC(=O)NC3=O)C4=O)cc2)C1] |
| 4b | 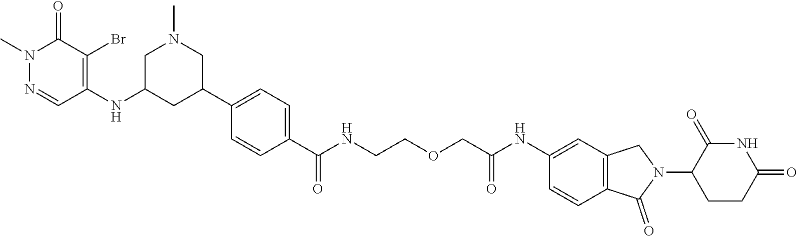 [SMILES: CN1CC(Nc2cnn(C)c(=O)c2Br)CC(c2ccc(C(=O)NCCOCC(=O)Nc3ccc4c(c3)CN(C3CCC(=O)NC3=O)C4=O)cc2)C1] |
| 5 | 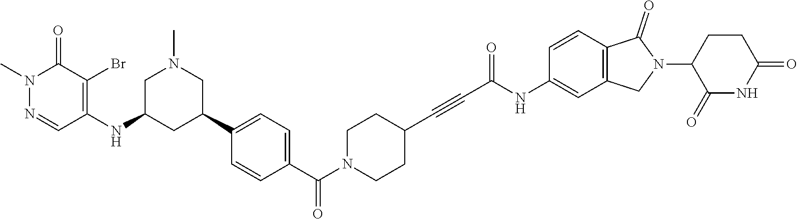 [SMILES: CN1C[C@H](Nc2cnn(C)c(=O)c2Br)C[C@H](c2ccc(C(=O)N3CCC(C#CC(=O)Nc4ccc5c(c4)CN(C4CCC(=O)NC4=O)C5=O)CC3)cc2)C1] |
| 5a | 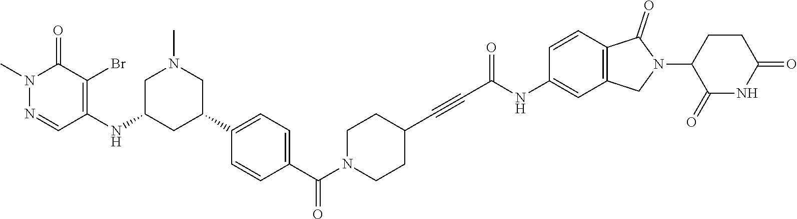 [SMILES: CN1C[C@@H](Nc2cnn(C)c(=O)c2Br)C[C@@H](c2ccc(C(=O)N3CCC(C#CC(=O)Nc4ccc5c(c4)CN(C4CCC(=O)NC4=O)C5=O)CC3)cc2)C1] |
| 5b | 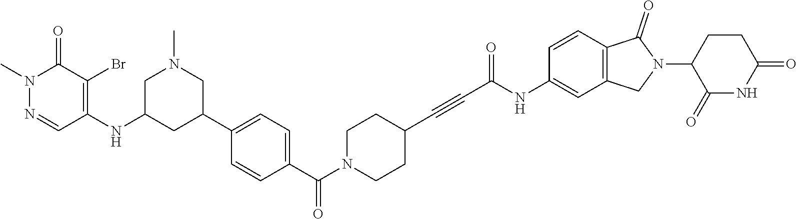 [SMILES: CN1CC(Nc2cnn(C)c(=O)c2Br)CC(c2ccc(C(=O)N3CCC(C#CC(=O)Nc4ccc5c(c4)CN(C4CCC(=O)NC4=O)C5=O)CC3)cc2)C1] |
| 6 | 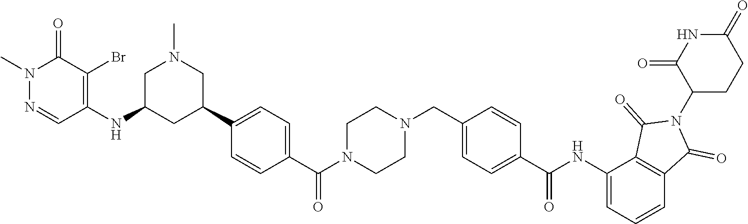 [SMILES: CN1C[C@H](Nc2cnn(C)c(=O)c2Br)C[C@H](c2ccc(C(=O)N3CCN(Cc4ccc(C(=O)Nc5cccc6c5C(=O)N(C5CCC(=O)NC5=O)C6=O)cc4)CC3)cc2)C1] |
| 6a | 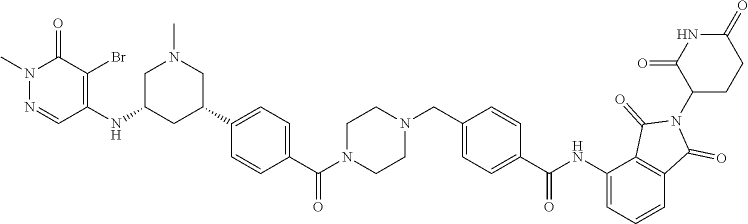 [SMILES: CN1C[C@@H](Nc2cnn(C)c(=O)c2Br)C[C@@H](c2ccc(C(=O)N3CCN(Cc4ccc(C(=O)Nc5cccc6c5C(=O)N(C5CCC(=O)NC5=O)C6=O)cc4)CC3)cc2)C1] |
| 6b | 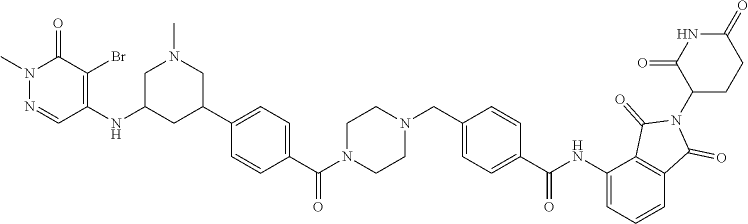 [SMILES: CN1CC(Nc2cnn(C)c(=O)c2Br)CC(c2ccc(C(=O)N3CCN(Cc4ccc(C(=O)Nc5cccc6c5C(=O)N(C5CCC(=O)NC5=O)C6=O)cc4)CC3)cc2)C1] |
| 7 | 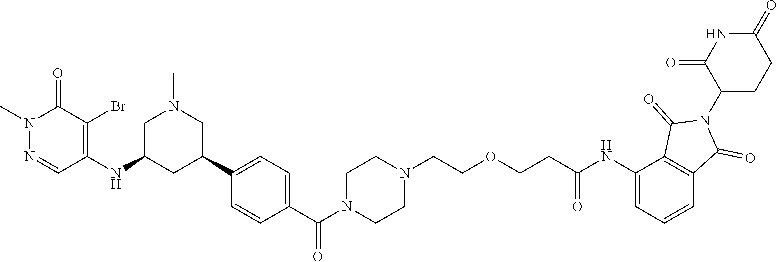 [SMILES: CN1C[C@H](Nc2cnn(C)c(=O)c2Br)C[C@H](c2ccc(C(=O)N3CCN(CCOCCC(=O)Nc4cccc5c4C(=O)N(C4CCC(=O)NC4=O)C5=O)CC3)cc2)C1] |
| 7a | 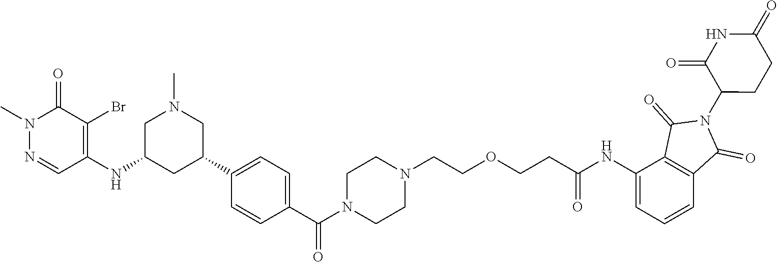 [SMILES: CN1C[C@@H](Nc2cnn(C)c(=O)c2Br)C[C@@H](c2ccc(C(=O)N3CCN(CCOCCC(=O)Nc4cccc5c4C(=O)N(C4CCC(=O)NC4=O)C5=O)CC3)cc2)C1] |
| 7b | 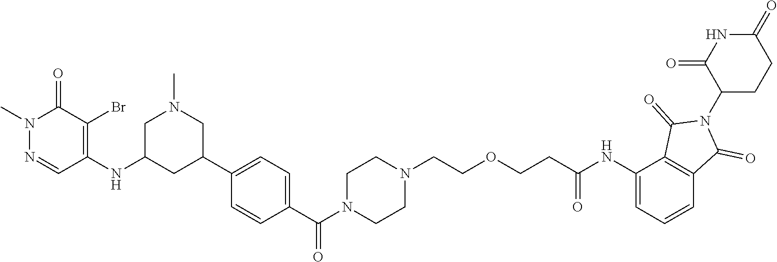 [SMILES: CN1CC(Nc2cnn(C)c(=O)c2Br)CC(c2ccc(C(=O)N3CCN(CCOCCC(=O)Nc4cccc5c4C(=O)N(C4CCC(=O)NC4=O)C5=O)CC3)cc2)C1] |
| 8 | 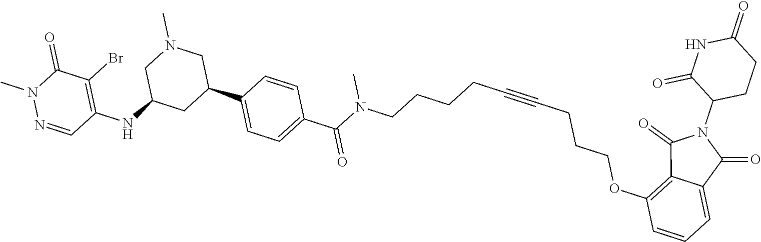 [SMILES: CN1C[C@H](Nc2cnn(C)c(=O)c2Br)C[C@H](c2ccc(C(=O)N(C)CCCCC#CCCCOc3cccc4c3C(=O)N(C3CCC(=O)NC3=O)C4=O)cc2)C1] |
| 8a | 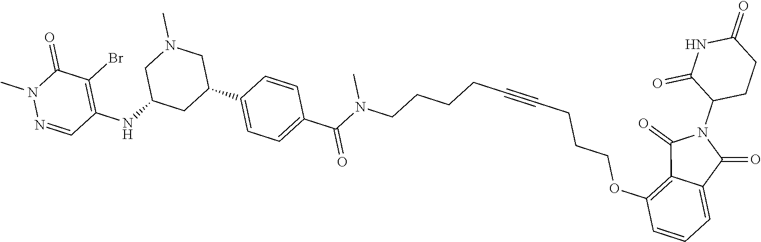 [SMILES: CN1C[C@@H](Nc2cnn(C)c(=O)c2Br)C[C@@H](c2ccc(C(=O)N(C)CCCCC#CCCCOc3cccc4c3C(=O)N(C3CCC(=O)NC3=O)C4=O)cc2)C1] |
| 8b | 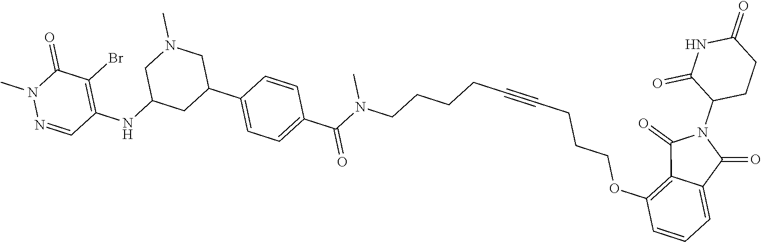 [SMILES: CN1CC(Nc2cnn(C)c(=O)c2Br)CC(c2ccc(C(=O)N(C)CCCCC#CCCCOc3cccc4c3C(=O)N(C3CCC(=O)NC3=O)C4=O)cc2)C1] |
| 9 | 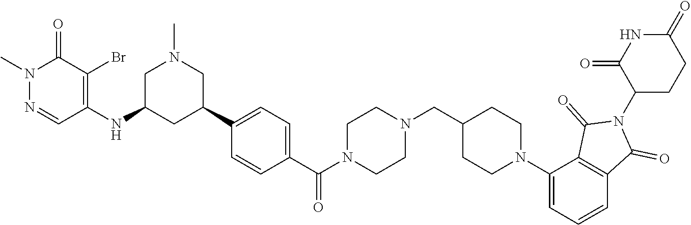 [SMILES: CN1C[C@H](Nc2cnn(C)c(=O)c2Br)C[C@H](c2ccc(C(=O)N3CCN(CC4CCN(c5cccc6c5C(=O)N(C5CCC(=O)NC5=O)C6=O)CC4)CC3)cc2)C1] |
| 9a | 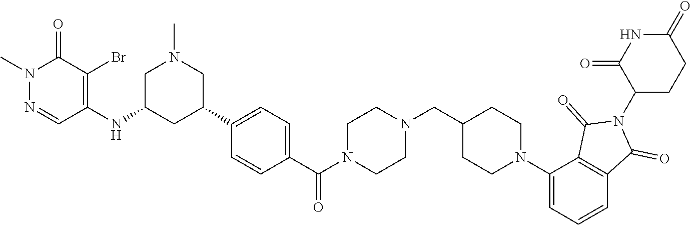 [SMILES: CN1C[C@@H](Nc2cnn(C)c(=O)c2Br)C[C@@H](c2ccc(C(=O)N3CCN(CC4CCN(c5cccc6c5C(=O)N(C5CCC(=O)NC5=O)C6=O)CC4)CC3)cc2)C1] |
| 9b | 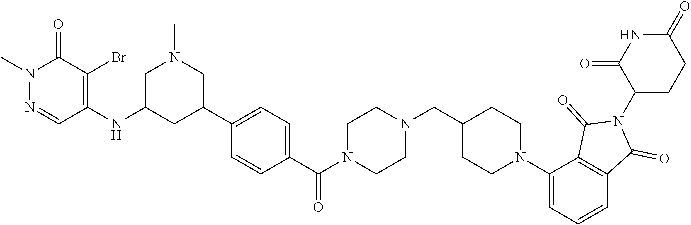 [SMILES: CN1CC(Nc2cnn(C)c(=O)c2Br)CC(c2ccc(C(=O)N3CCN(CC4CCN(c5cccc6c5C(=O)N(C5CCC(=O)NC5=O)C6=O)CC4)CC3)cc2)C1] |
| 10 | 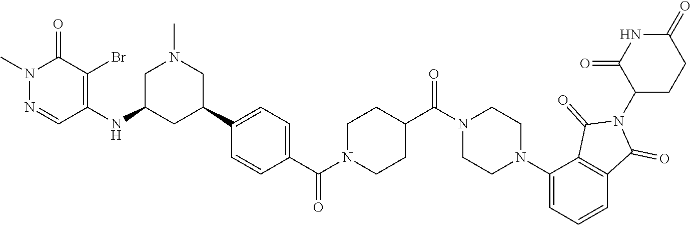 [SMILES: CN1C[C@H](Nc2cnn(C)c(=O)c2Br)C[C@H](c2ccc(C(=O)N3CCC(C(=O)N4CCN(c5cccc6c5C(=O)N(C5CCC(=O)NC5=O)C6=O)CC4)CC3)cc2)C1] |
| 10a | 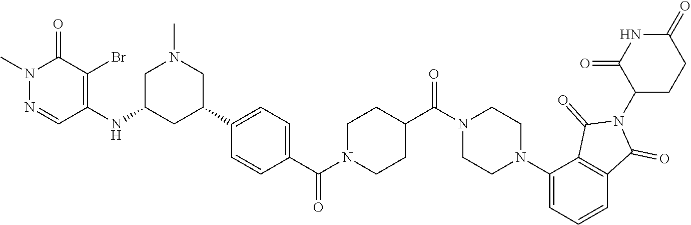 [SMILES: CN1C[C@@H](Nc2cnn(C)c(=O)c2Br)C[C@@H](c2ccc(C(=O)N3CCC(C(=O)N4CCN(c5cccc6c5C(=O)N(C5CCC(=O)NC5=O)C6=O)CC4)CC3)cc2)C1] |
| 10b | 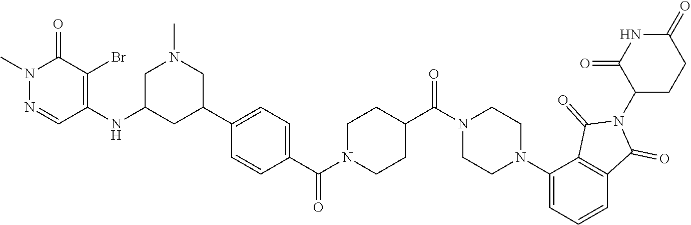 [SMILES: CN1CC(Nc2cnn(C)c(=O)c2Br)CC(c2ccc(C(=O)N3CCC(C(=O)N4CCN(c5cccc6c5C(=O)N(C5CCC(=O)NC5=O)C6=O)CC4)CC3)cc2)C1] |
| 11 | 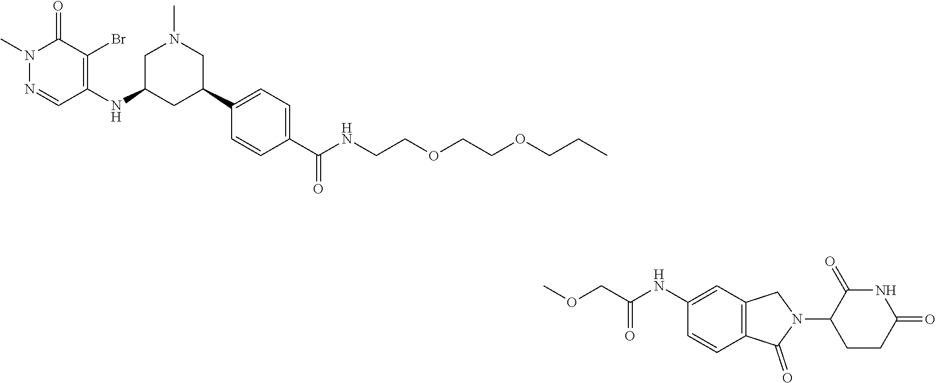 |
| 11a | 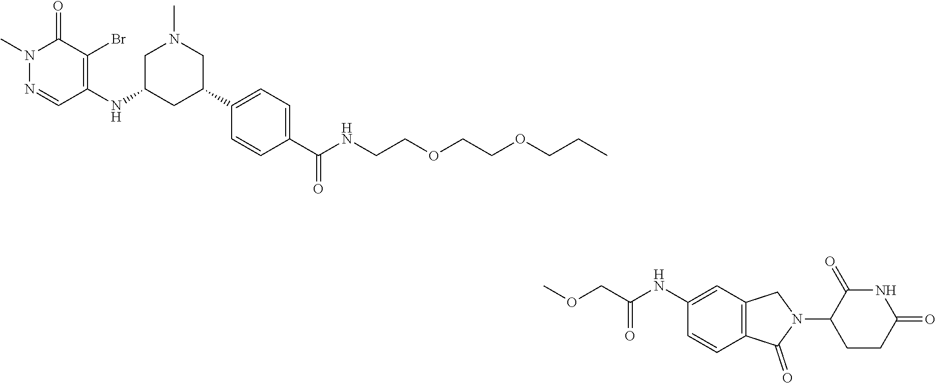 |
| 11b | 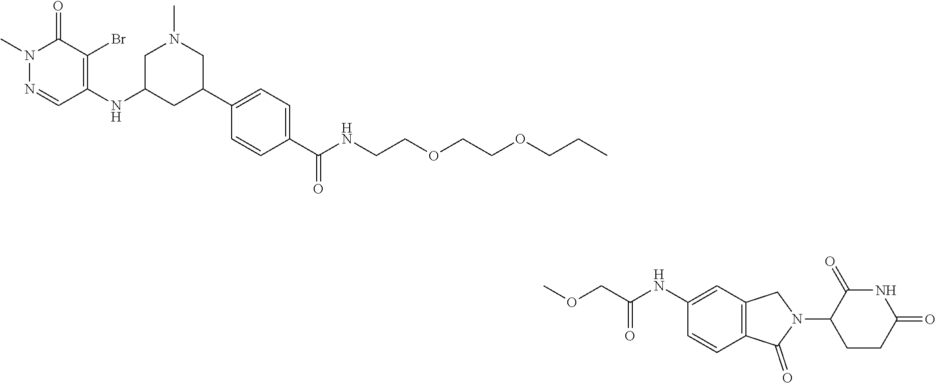 |
| 12 | 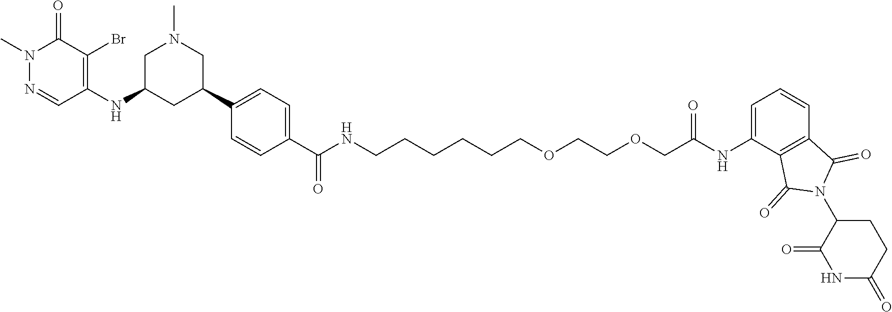 [SMILES: CN1C[C@H](Nc2cnn(C)c(=O)c2Br)C[C@H](c2ccc(C(=O)NCCCCCCOCCOCC(=O)Nc3cccc4c3C(=O)N(C3CCC(=O)NC3=O)C4=O)cc2)C1] |
| 12a | 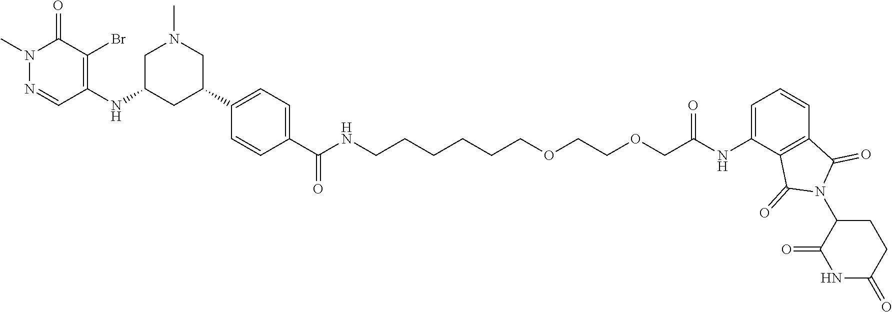 [SMILES: CN1C[C@@H](Nc2cnn(C)c(=O)c2Br)C[C@@H](c2ccc(C(=O)NCCCCCCOCCOCC(=O)Nc3cccc4c3C(=O)N(C3CCC(=O)NC3=O)C4=O)cc2)C1] |
| 12b | 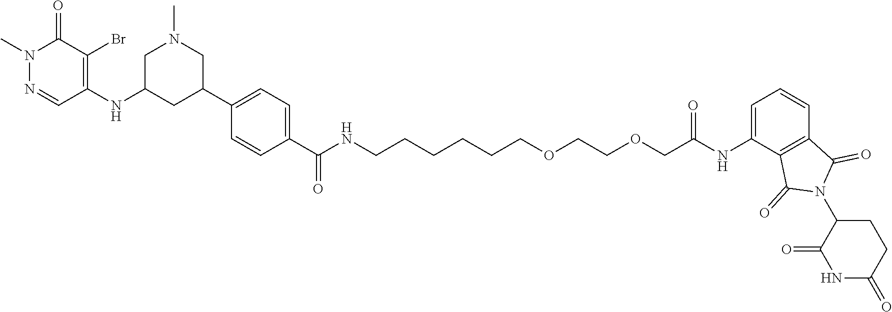 [SMILES: CN1CC(Nc2cnn(C)c(=O)c2Br)CC(c2ccc(C(=O)NCCCCCCOCCOCC(=O)Nc3cccc4c3C(=O)N(C3CCC(=O)NC3=O)C4=O)cc2)C1] |
| 13 | 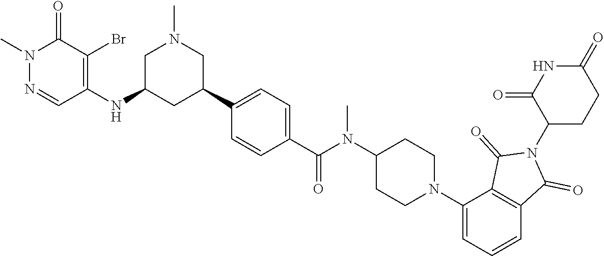 [SMILES: CN1C[C@H](Nc2cnn(C)c(=O)c2Br)C[C@H](c2ccc(C(=O)N(C)C3CCN(c4cccc5c4C(=O)N(C4CCC(=O)NC4=O)C5=O)CC3)cc2)C1] |
| 13a | 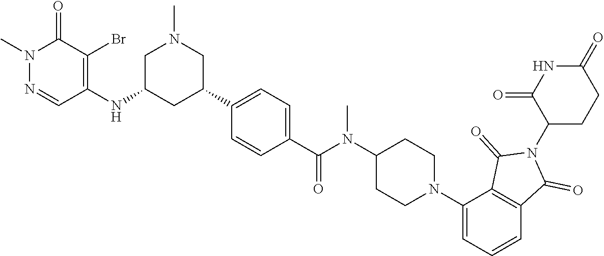 [SMILES: CN1C[C@@H](Nc2cnn(C)c(=O)c2Br)C[C@@H](c2ccc(C(=O)N(C)C3CCN(c4cccc5c4C(=O)N(C4CCC(=O)NC4=O)C5=O)CC3)cc2)C1] |
| 13b | 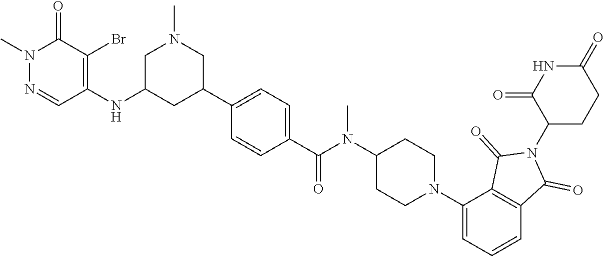 [SMILES: CN1CC(Nc2cnn(C)c(=O)c2Br)CC(c2ccc(C(=O)N(C)C3CCN(c4cccc5c4C(=O)N(C4CCC(=O)NC4=O)C5=O)CC3)cc2)C1] |
| 14 | 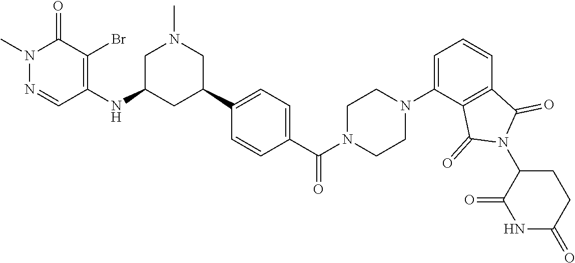 [SMILES: CN1C[C@H](Nc2cnn(C)c(=O)c2Br)C[C@H](c2ccc(C(=O)N3CCN(c4cccc5c4C(=O)N(C4CCC(=O)NC4=O)C5=O)CC3)cc2)C1] |
| 14a | 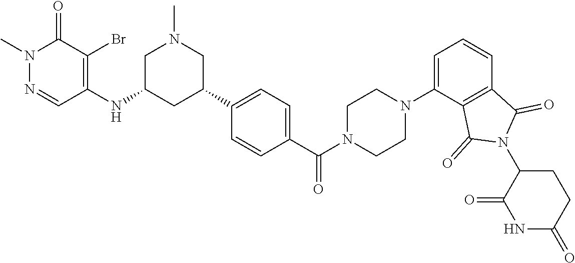 [SMILES: CN1C[C@@H](Nc2cnn(C)c(=O)c2Br)C[C@@H](c2ccc(C(=O)N3CCN(c4cccc5c4C(=O)N(C4CCC(=O)NC4=O)C5=O)CC3)cc2)C1] |
| 14b | 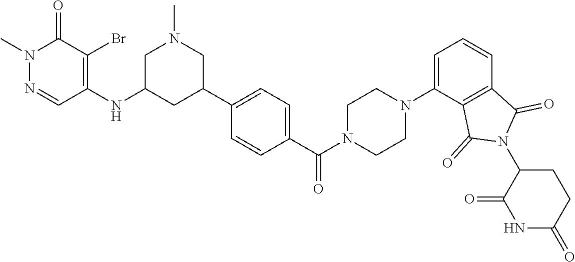 [SMILES: CN1CC(Nc2cnn(C)c(=O)c2Br)CC(c2ccc(C(=O)N3CCN(c4cccc5c4C(=O)N(C4CCC(=O)NC4=O)C5=O)CC3)cc2)C1] |
| 15 | 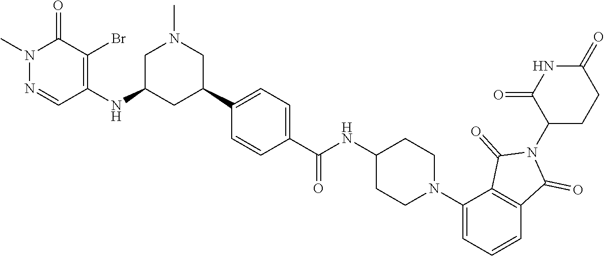 [SMILES: CN1C[C@H](Nc2cnn(C)c(=O)c2Br)C[C@H](c2ccc(C(=O)NC3CCN(c4cccc5c4C(=O)N(C4CCC(=O)NC4=O)C5=O)CC3)cc2)C1] |
| 15a | 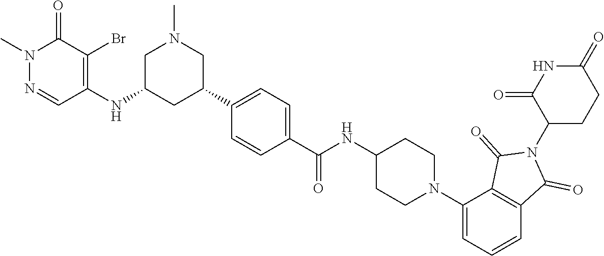 [SMILES: CN1C[C@@H](Nc2cnn(C)c(=O)c2Br)C[C@@H](c2ccc(C(=O)NC3CCN(c4cccc5c4C(=O)N(C4CCC(=O)NC4=O)C5=O)CC3)cc2)C1] |
| 15b | 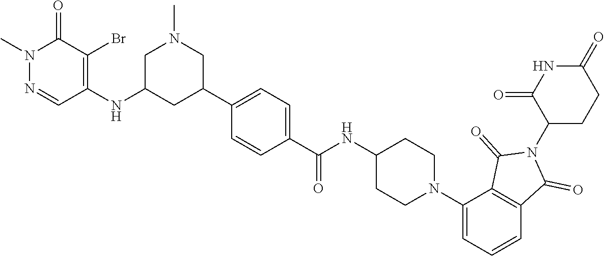 [SMILES: CN1CC(Nc2cnn(C)c(=O)c2Br)CC(c2ccc(C(=O)NC3CCN(c4cccc5c4C(=O)N(C4CCC(=O)NC4=O)C5=O)CC3)cc2)C1] |
| 16 | 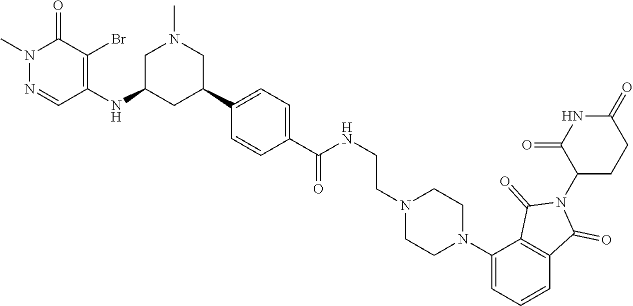 [SMILES: CN1C[C@H](Nc2cnn(C)c(=O)c2Br)C[C@H](c2ccc(C(=O)NCCN3CCN(c4cccc5c4C(=O)N(C4CCC(=O)NC4=O)C5=O)CC3)cc2)C1] |
| 16a | 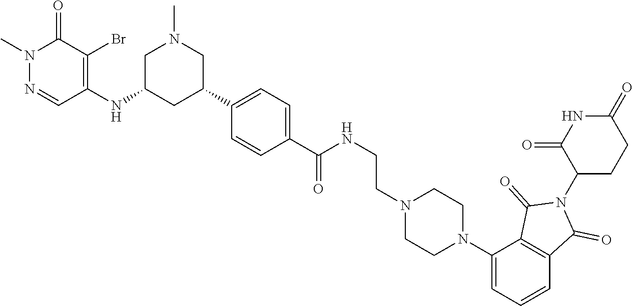 [SMILES: CN1C[C@@H](Nc2cnn(C)c(=O)c2Br)C[C@@H](c2ccc(C(=O)NCCN3CCN(c4cccc5c4C(=O)N(C4CCC(=O)NC4=O)C5=O)CC3)cc2)C1] |
| 16b | 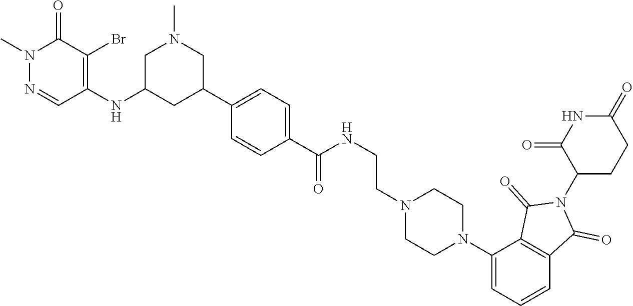 [SMILES: CN1CC(Nc2cnn(C)c(=O)c2Br)CC(c2ccc(C(=O)NCCN3CCN(c4cccc5c4C(=O)N(C4CCC(=O)NC4=O)C5=O)CC3)cc2)C1] |
| 17 | 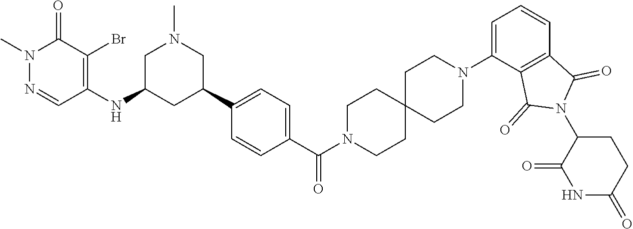 [SMILES: CN1C[C@H](Nc2cnn(C)c(=O)c2Br)C[C@H](c2ccc(C(=O)N3CCC4(CC3)CCN(c3cccc5c3C(=O)N(C3CCC(=O)NC3=O)C5=O)CC4)cc2)C1] |
| 17a | 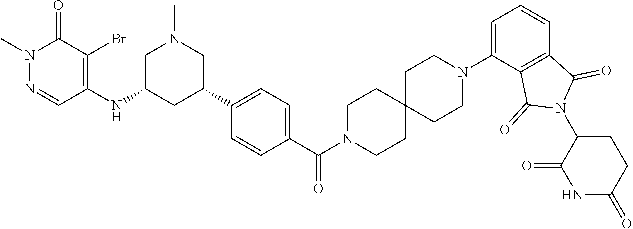 [SMILES: CN1C[C@@H](Nc2cnn(C)c(=O)c2Br)C[C@@H](c2ccc(C(=O)N3CCC4(CC3)CCN(c3cccc5c3C(=O)N(C3CCC(=O)NC3=O)C5=O)CC4)cc2)C1] |
| 17b | 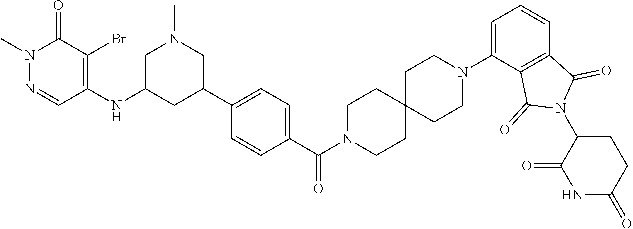 [SMILES: CN1CC(Nc2cnn(C)c(=O)c2Br)CC(c2ccc(C(=O)N3CCC4(CC3)CCN(c3cccc5c3C(=O)N(C3CCC(=O)NC3=O)C5=O)CC4)cc2)C1] |
| 18 | 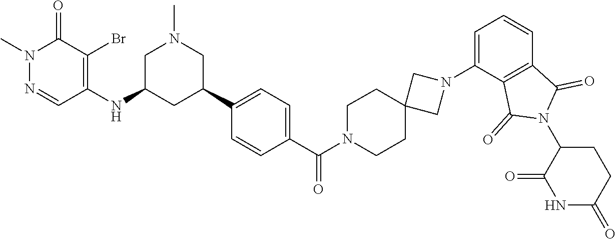 [SMILES: CN1C[C@H](Nc2cnn(C)c(=O)c2Br)C[C@H](c2ccc(C(=O)N3CCC4(CC3)CN(c3cccc5c3C(=O)N(C3CCC(=O)NC3=O)C5=O)C4)cc2)C1] |
| 18a | 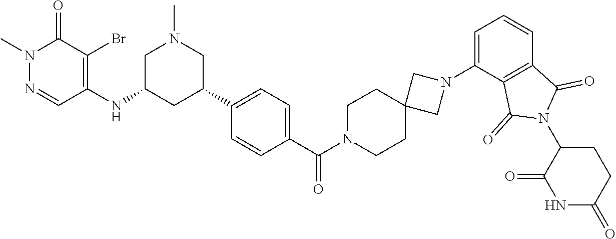 [SMILES: CN1C[C@@H](Nc2cnn(C)c(=O)c2Br)C[C@@H](c2ccc(C(=O)N3CCC4(CC3)CN(c3cccc5c3C(=O)N(C3CCC(=O)NC3=O)C5=O)C4)cc2)C1] |
| 18b | 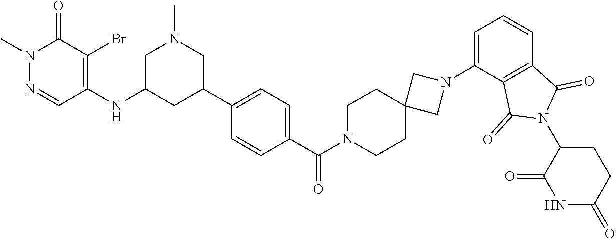 [SMILES: CN1CC(Nc2cnn(C)c(=O)c2Br)CC(c2ccc(C(=O)N3CCC4(CC3)CN(c3cccc5c3C(=O)N(C3CCC(=O)NC3=O)C5=O)C4)cc2)C1] |
| 19 | 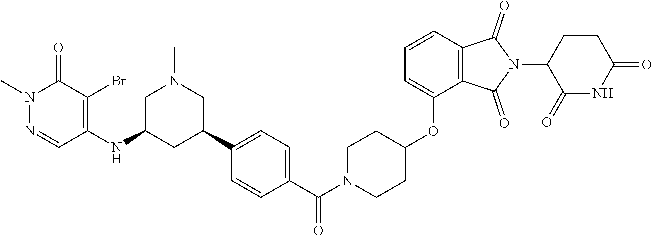 [SMILES: CN1C[C@H](Nc2cnn(C)c(=O)c2Br)C[C@H](c2ccc(C(=O)N3CCC(Oc4cccc5c4C(=O)N(C4CCC(=O)NC4=O)C5=O)CC3)cc2)C1] |
| 19a | 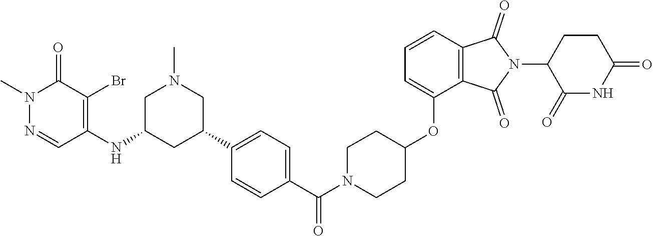 [SMILES: CN1C[C@@H](Nc2cnn(C)c(=O)c2Br)C[C@@H](c2ccc(C(=O)N3CCC(Oc4cccc5c4C(=O)N(C4CCC(=O)NC4=O)C5=O)CC3)cc2)C1] |
| 19b | 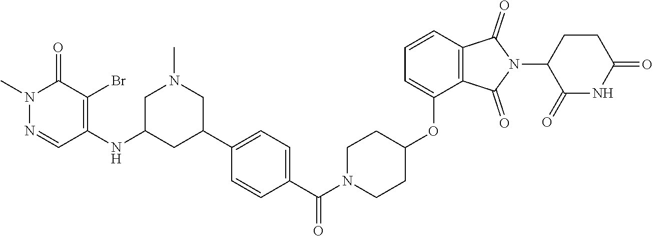 [SMILES: CN1CC(Nc2cnn(C)c(=O)c2Br)CC(c2ccc(C(=O)N3CCC(Oc4cccc5c4C(=O)N(C4CCC(=O)NC4=O)C5=O)CC3)cc2)C1] |
| 20 | 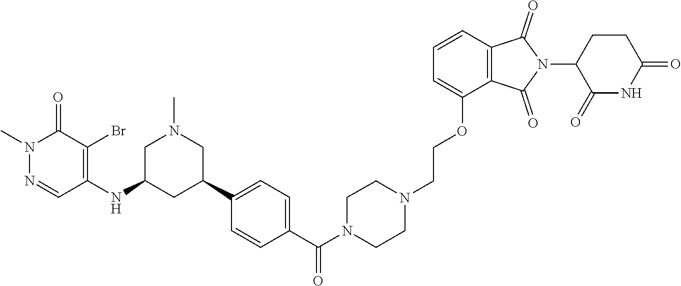 [SMILES: CN1C[C@H](Nc2cnn(C)c(=O)c2Br)C[C@H](c2ccc(C(=O)N3CCN(CCOc4cccc5c4C(=O)N(C4CCC(=O)NC4=O)C5=O)CC3)cc2)C1] |
| 20a | 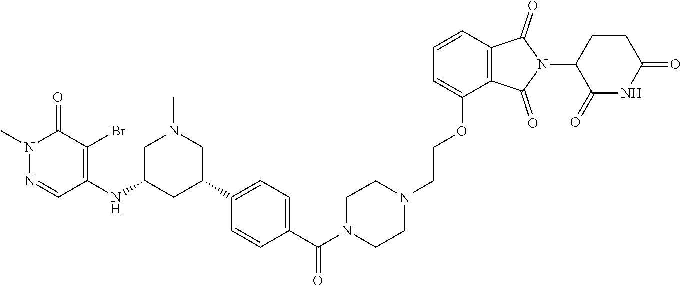 [SMILES: CN1C[C@@H](Nc2cnn(C)c(=O)c2Br)C[C@@H](c2ccc(C(=O)N3CCN(CCOc4cccc5c4C(=O)N(C4CCC(=O)NC4=O)C5=O)CC3)cc2)C1] |
| 20b | 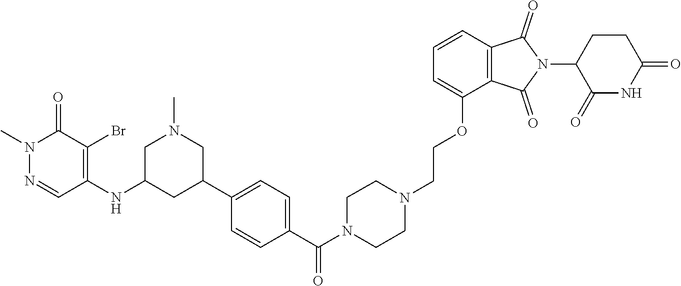 [SMILES: CN1CC(Nc2cnn(C)c(=O)c2Br)CC(c2ccc(C(=O)N3CCN(CCOc4cccc5c4C(=O)N(C4CCC(=O)NC4=O)C5=O)CC3)cc2)C1] |
| 21 | 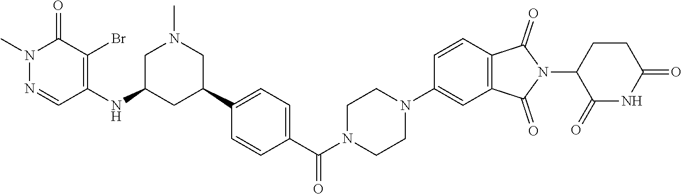 [SMILES: CN1C[C@H](Nc2cnn(C)c(=O)c2Br)C[C@H](c2ccc(C(=O)N3CCN(c4ccc5c(c4)C(=O)N(C4CCC(=O)NC4=O)C5=O)CC3)cc2)C1] |
| 21a | 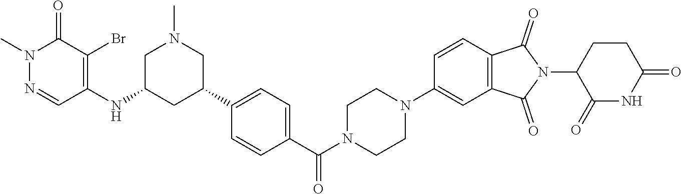 [SMILES: CN1C[C@@H](Nc2cnn(C)c(=O)c2Br)C[C@@H](c2ccc(C(=O)N3CCN(c4ccc5c(c4)C(=O)N(C4CCC(=O)NC4=O)C5=O)CC3)cc2)C1] |
| 21b | 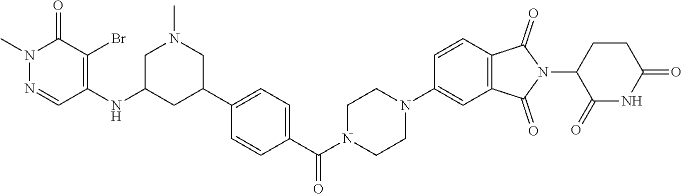 [SMILES: CN1CC(Nc2cnn(C)c(=O)c2Br)CC(c2ccc(C(=O)N3CCN(c4ccc5c(c4)C(=O)N(C4CCC(=O)NC4=O)C5=O)CC3)cc2)C1] |
| 22 | 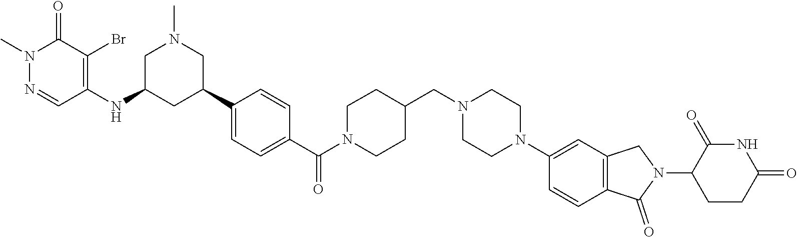 [SMILES: CN1C[C@H](Nc2cnn(C)c(=O)c2Br)C[C@H](c2ccc(C(=O)N3CCC(CN4CCN(c5ccc6c(c5)CN(C5CCC(=O)NC5=O)C6=O)CC4)CC3)cc2)C1] |
| 22-1 | 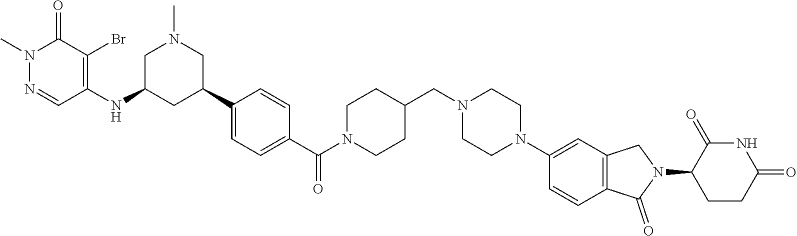 [SMILES: CN1C[C@H](Nc2cnn(C)c(=O)c2Br)C[C@H](c2ccc(C(=O)N3CCC(CN4CCN(c5ccc6c(c5)CN([C@@H]5CCC(=O)NC5=O)C6=O)CC4)CC3)cc2)C1] |
| 22-2 | 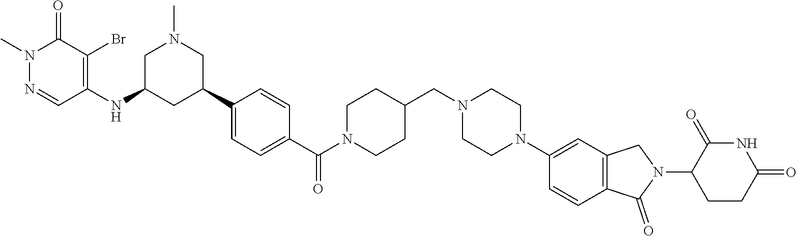 [SMILES: CN1C[C@H](Nc2cnn(C)c(=O)c2Br)C[C@H](c2ccc(C(=O)N3CCC(CN4CCN(c5ccc6c(c5)CN(C5CCC(=O)NC5=O)C6=O)CC4)CC3)cc2)C1] |
| 22a | 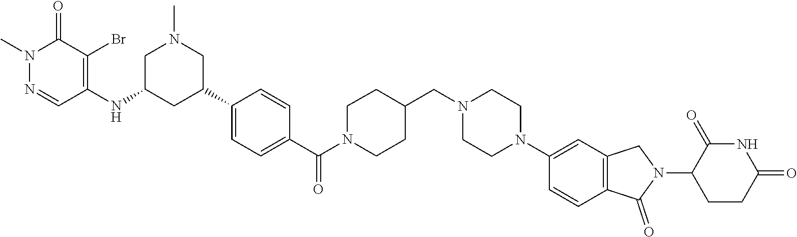 |
| 22b | 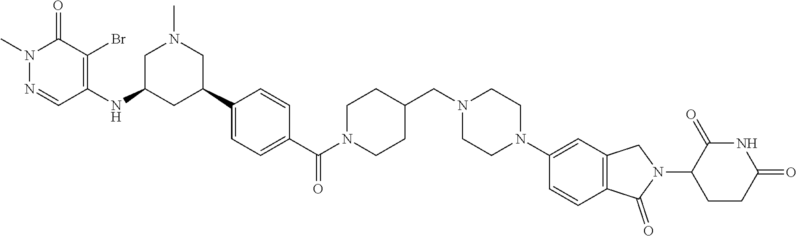 [SMILES: CN1C[C@H](Nc2cnn(C)c(=O)c2Br)C[C@H](c2ccc(C(=O)N3CCC(CN4CCN(c5ccc6c(c5)CN(C5CCC(=O)NC5=O)C6=O)CC4)CC3)cc2)C1] |
| 23 | 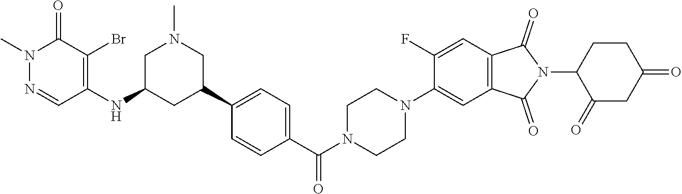 |
| 23a | 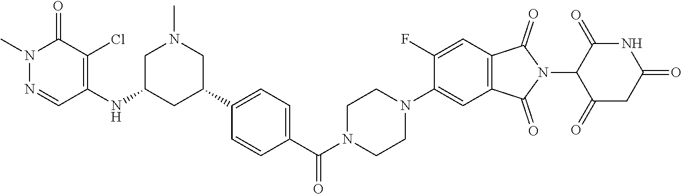 [SMILES: CN1C[C@@H](Nc2cnn(C)c(=O)c2Cl)C[C@@H](c2ccc(C(=O)N3CCN(c4cc5c(cc4F)C(=O)N(C4C(=O)CC(=O)NC4=O)C5=O)CC3)cc2)C1] |
| 23b | 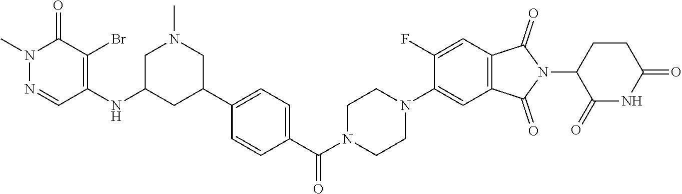 [SMILES: CN1CC(Nc2cnn(C)c(=O)c2Br)CC(c2ccc(C(=O)N3CCN(c4cc5c(cc4F)C(=O)N(C4CCC(=O)NC4=O)C5=O)CC3)cc2)C1] |
| 24 | 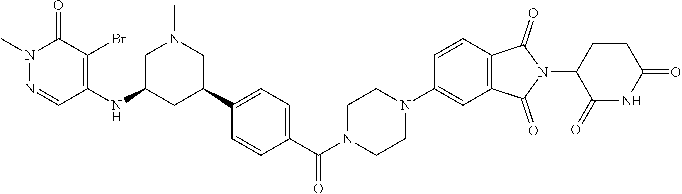 [SMILES: CN1C[C@H](Nc2cnn(C)c(=O)c2Br)C[C@H](c2ccc(C(=O)N3CCN(c4ccc5c(c4)C(=O)N(C4CCC(=O)NC4=O)C5=O)CC3)cc2)C1] |
| 24a | 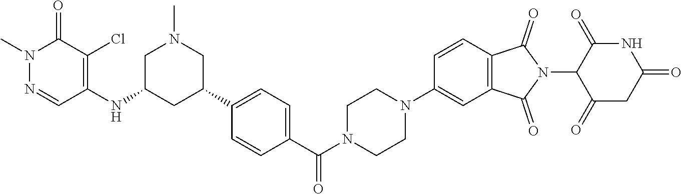 [SMILES: CN1C[C@@H](Nc2cnn(C)c(=O)c2Cl)C[C@@H](c2ccc(C(=O)N3CCN(c4ccc5c(c4)C(=O)N(C4C(=O)CC(=O)NC4=O)C5=O)CC3)cc2)C1] |
| 24b | 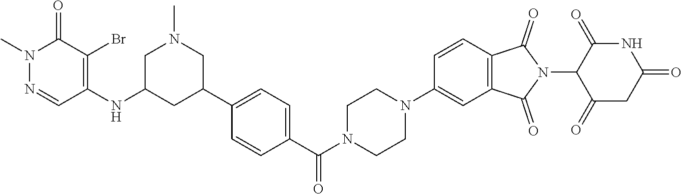 [SMILES: CN1CC(Nc2cnn(C)c(=O)c2Br)CC(c2ccc(C(=O)N3CCN(c4ccc5c(c4)C(=O)N(C4C(=O)CC(=O)NC4=O)C5=O)CC3)cc2)C1] |
| 25 | 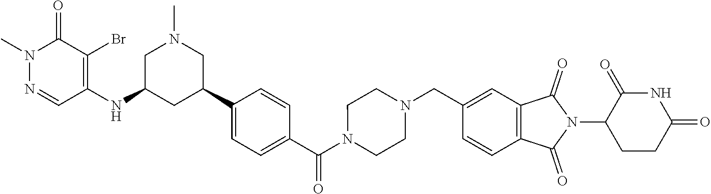 [SMILES: CN1C[C@H](Nc2cnn(C)c(=O)c2Br)C[C@H](c2ccc(C(=O)N3CCN(Cc4ccc5c(c4)C(=O)N(C4CCC(=O)NC4=O)C5=O)CC3)cc2)C1] |
| 25a | 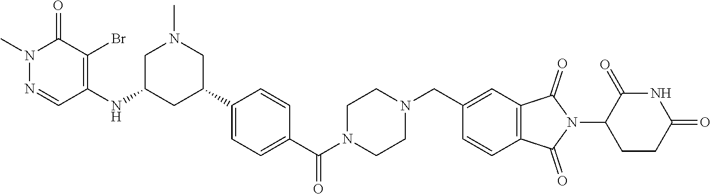 [SMILES: CN1C[C@@H](Nc2cnn(C)c(=O)c2Br)C[C@@H](c2ccc(C(=O)N3CCN(Cc4ccc5c(c4)C(=O)N(C4CCC(=O)NC4=O)C5=O)CC3)cc2)C1] |
| 25b | 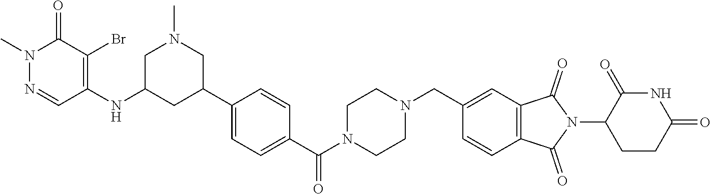 [SMILES: CN1CC(Nc2cnn(C)c(=O)c2Br)CC(c2ccc(C(=O)N3CCN(Cc4ccc5c(c4)C(=O)N(C4CCC(=O)NC4=O)C5=O)CC3)cc2)C1] |
| 26 | 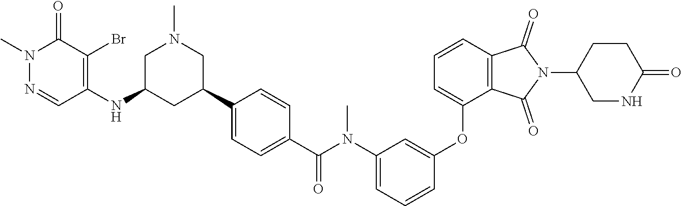 [SMILES: CN1C[C@H](Nc2cnn(C)c(=O)c2Br)C[C@H](c2ccc(C(=O)N(C)c3cccc(Oc4cccc5c4C(=O)N(C4CCC(=O)NC4)C5=O)c3)cc2)C1] |
| 26a | 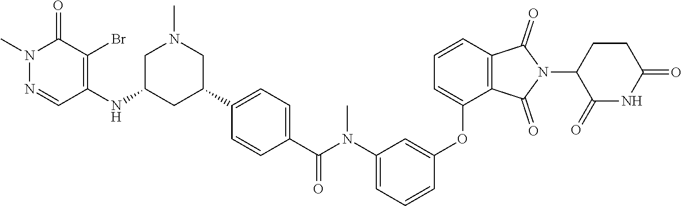 [SMILES: CN1C[C@@H](Nc2cnn(C)c(=O)c2Br)C[C@@H](c2ccc(C(=O)N(C)c3cccc(Oc4cccc5c4C(=O)N(C4CCC(=O)NC4=O)C5=O)c3)cc2)C1] |
| 26b | 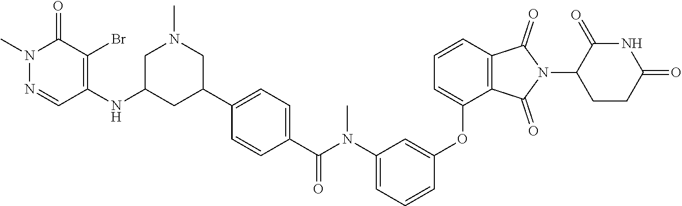 [SMILES: CN1CC(Nc2cnn(C)c(=O)c2Br)CC(c2ccc(C(=O)N(C)c3cccc(Oc4cccc5c4C(=O)N(C4CCC(=O)NC4=O)C5=O)c3)cc2)C1] |
| 27 | 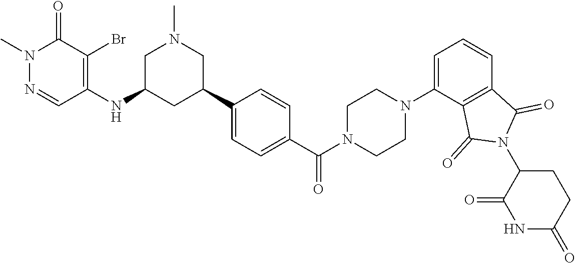 [SMILES: CN1C[C@H](Nc2cnn(C)c(=O)c2Br)C[C@H](c2ccc(C(=O)N3CCN(c4cccc5c4C(=O)N(C4CCC(=O)NC4=O)C5=O)CC3)cc2)C1] |
| 27a | 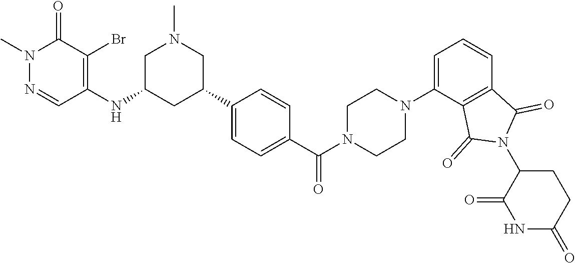 [SMILES: CN1C[C@@H](Nc2cnn(C)c(=O)c2Br)C[C@@H](c2ccc(C(=O)N3CCN(c4cccc5c4C(=O)N(C4CCC(=O)NC4=O)C5=O)CC3)cc2)C1] |
| 27b | 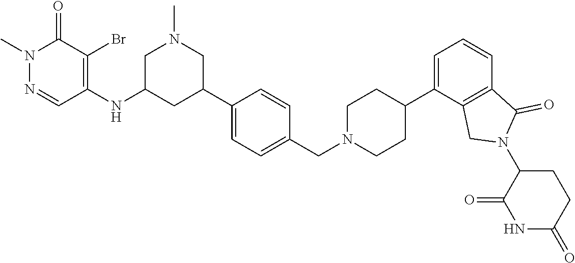 [SMILES: CN1CC(Nc2cnn(C)c(=O)c2Br)CC(c2ccc(CN3CCC(c4cccc5c4CN(C4CCC(=O)NC4=O)C5=O)CC3)cc2)C1] |
| 28 | 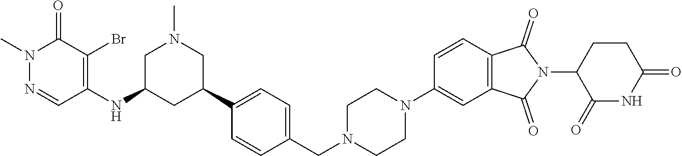 [SMILES: CN1C[C@H](Nc2cnn(C)c(=O)c2Br)C[C@H](c2ccc(CN3CCN(c4ccc5c(c4)C(=O)N(C4CCC(=O)NC4=O)C5=O)CC3)cc2)C1] |
| 28a | 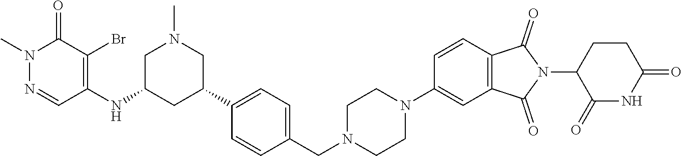 [SMILES: CN1C[C@@H](Nc2cnn(C)c(=O)c2Br)C[C@@H](c2ccc(CN3CCN(c4ccc5c(c4)C(=O)N(C4CCC(=O)NC4=O)C5=O)CC3)cc2)C1] |
| 28b | 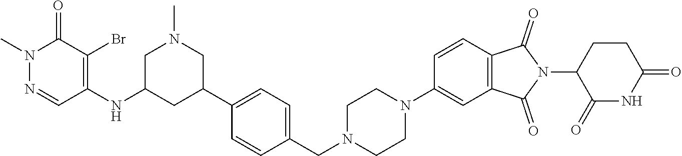 [SMILES: CN1CC(Nc2cnn(C)c(=O)c2Br)CC(c2ccc(CN3CCN(c4ccc5c(c4)C(=O)N(C4CCC(=O)NC4=O)C5=O)CC3)cc2)C1] |
| 29 | 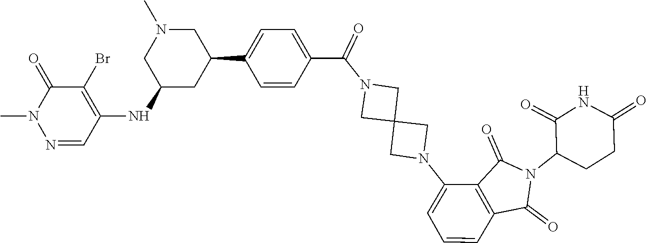 [SMILES: CN1C[C@H](Nc2cnn(C)c(=O)c2Br)C[C@H](c2ccc(C(=O)N3CC4(C3)CN(c3cccc5c3C(=O)N(C3CCC(=O)NC3=O)C5=O)C4)cc2)C1] |
| 29a | 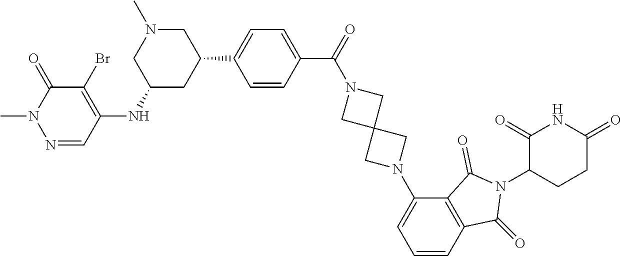 [SMILES: CN1C[C@@H](Nc2cnn(C)c(=O)c2Br)C[C@@H](c2ccc(C(=O)N3CC4(C3)CN(c3cccc5c3C(=O)N(C3CCC(=O)NC3=O)C5=O)C4)cc2)C1] |
| 29b | 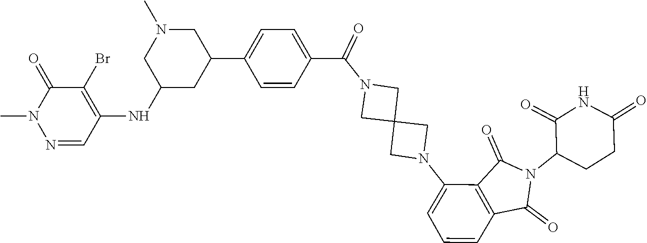 [SMILES: CN1CC(Nc2cnn(C)c(=O)c2Br)CC(c2ccc(C(=O)N3CC4(C3)CN(c3cccc5c3C(=O)N(C3CCC(=O)NC3=O)C5=O)C4)cc2)C1] |
| 30 | 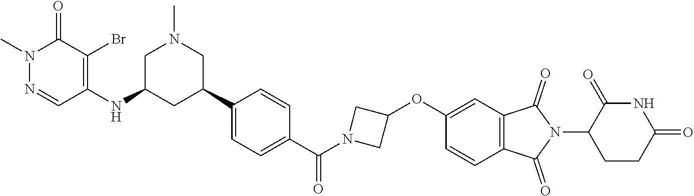 [SMILES: CN1C[C@H](Nc2cnn(C)c(=O)c2Br)C[C@H](c2ccc(C(=O)N3CC(Oc4ccc5c(c4)C(=O)N(C4CCC(=O)NC4=O)C5=O)C3)cc2)C1] |
| 30a | 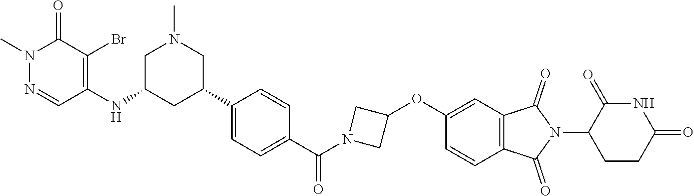 [SMILES: CN1C[C@@H](Nc2cnn(C)c(=O)c2Br)C[C@@H](c2ccc(C(=O)N3CC(Oc4ccc5c(c4)C(=O)N(C4CCC(=O)NC4=O)C5=O)C3)cc2)C1] |
| 30b | 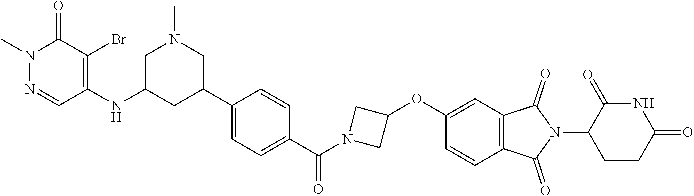 [SMILES: CN1CC(Nc2cnn(C)c(=O)c2Br)CC(c2ccc(C(=O)N3CC(Oc4ccc5c(c4)C(=O)N(C4CCC(=O)NC4=O)C5=O)C3)cc2)C1] |
| 31 | 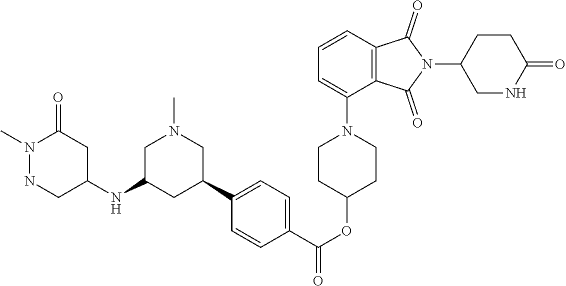 |
| 31a | 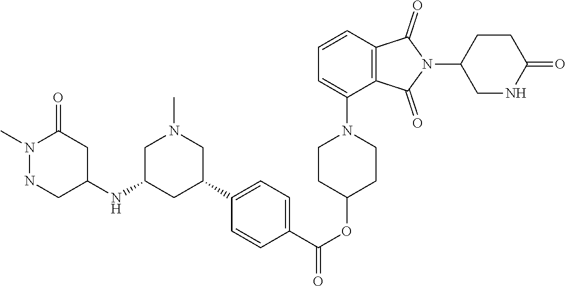 [SMILES: CN1C[C@@H](NC2CNN(C)C(=O)C2)C[C@@H](c2ccc(C(=O)OC3CCN(c4cccc5c4C(=O)N(C4CCC(=O)NC4)C5=O)CC3)cc2)C1] |
| 31b | 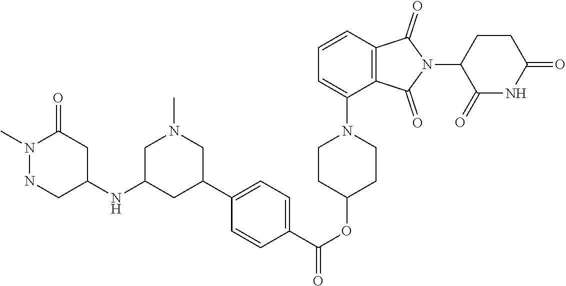 [SMILES: CN1CC(NC2CNN(C)C(=O)C2)CC(c2ccc(C(=O)OC3CCN(c4cccc5c4C(=O)N(C4CCC(=O)NC4=O)C5=O)CC3)cc2)C1] |
| 32 | 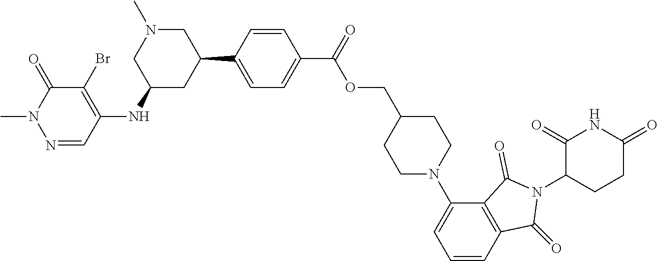 [SMILES: CN1C[C@H](Nc2cnn(C)c(=O)c2Br)C[C@H](c2ccc(C(=O)OCC3CCN(c4cccc5c4C(=O)N(C4CCC(=O)NC4=O)C5=O)CC3)cc2)C1] |
| 32a | 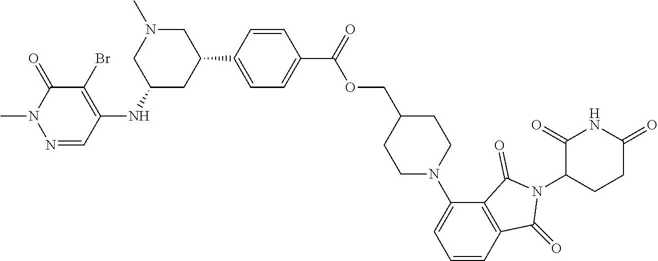 [SMILES: CN1C[C@@H](Nc2cnn(C)c(=O)c2Br)C[C@@H](c2ccc(C(=O)OCC3CCN(c4cccc5c4C(=O)N(C4CCC(=O)NC4=O)C5=O)CC3)cc2)C1] |
| 32b | 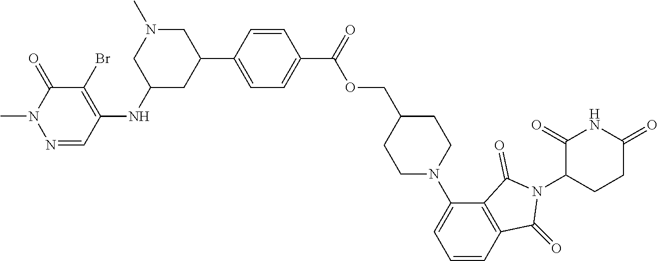 [SMILES: CN1CC(Nc2cnn(C)c(=O)c2Br)CC(c2ccc(C(=O)OCC3CCN(c4cccc5c4C(=O)N(C4CCC(=O)NC4=O)C5=O)CC3)cc2)C1] |
| 33 | 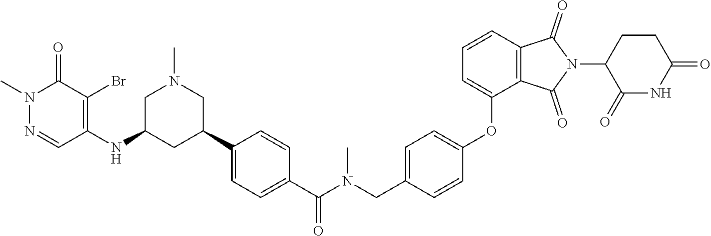 [SMILES: CN1C[C@H](Nc2cnn(C)c(=O)c2Br)C[C@H](c2ccc(C(=O)N(C)Cc3ccc(Oc4cccc5c4C(=O)N(C4CCC(=O)NC4=O)C5=O)cc3)cc2)C1] |
| 33a | 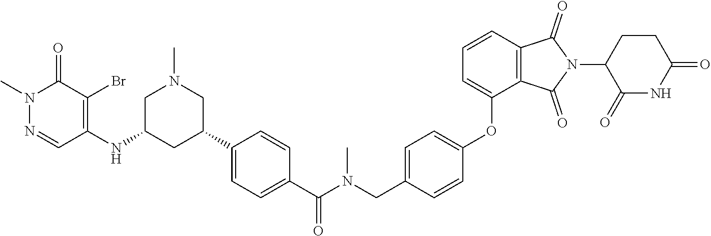 [SMILES: CN1C[C@@H](Nc2cnn(C)c(=O)c2Br)C[C@@H](c2ccc(C(=O)N(C)Cc3ccc(Oc4cccc5c4C(=O)N(C4CCC(=O)NC4=O)C5=O)cc3)cc2)C1] |
| 33b | 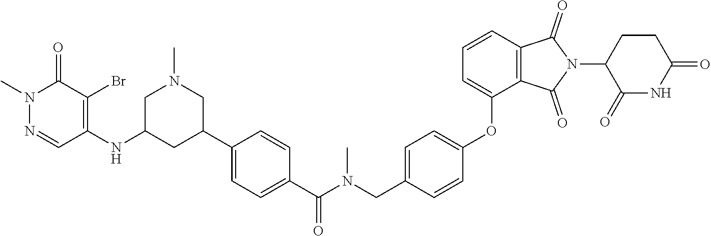 [SMILES: CN1CC(Nc2cnn(C)c(=O)c2Br)CC(c2ccc(C(=O)N(C)Cc3ccc(Oc4cccc5c4C(=O)N(C4CCC(=O)NC4=O)C5=O)cc3)cc2)C1] |
| 34 | 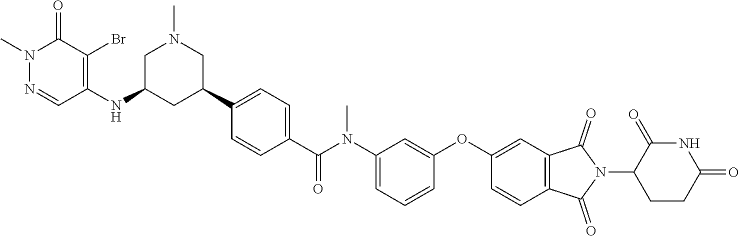 [SMILES: CN1C[C@H](Nc2cnn(C)c(=O)c2Br)C[C@H](c2ccc(C(=O)N(C)c3cccc(Oc4ccc5c(c4)C(=O)N(C4CCC(=O)NC4=O)C5=O)c3)cc2)C1] |
| 34a | 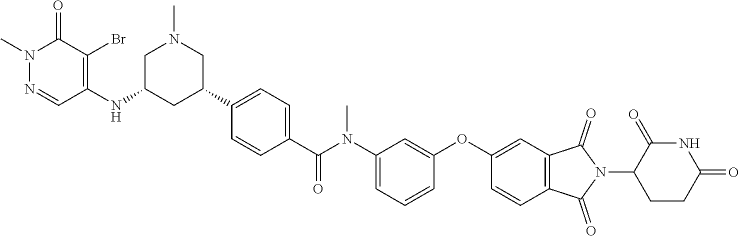 [SMILES: CN1C[C@@H](Nc2cnn(C)c(=O)c2Br)C[C@@H](c2ccc(C(=O)N(C)c3cccc(Oc4ccc5c(c4)C(=O)N(C4CCC(=O)NC4=O)C5=O)c3)cc2)C1] |
| 34b | 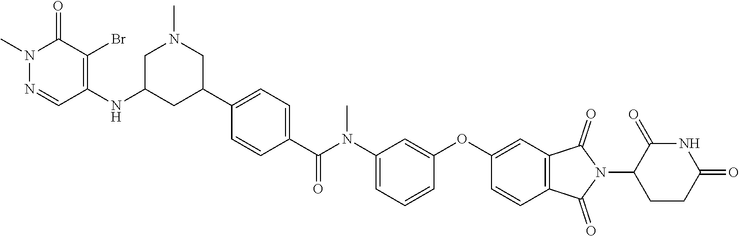 [SMILES: CN1CC(Nc2cnn(C)c(=O)c2Br)CC(c2ccc(C(=O)N(C)c3cccc(Oc4ccc5c(c4)C(=O)N(C4CCC(=O)NC4=O)C5=O)c3)cc2)C1] |
| 35 | 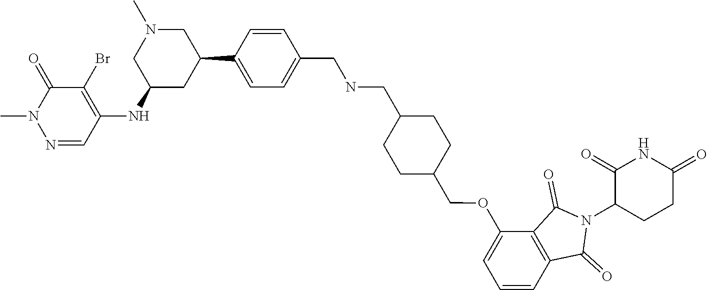 [SMILES: CN1C[C@H](Nc2cnn(C)c(=O)c2Br)C[C@H](c2ccc(CNCC3CCC(COc4cccc5c4C(=O)N(C4CCC(=O)NC4=O)C5=O)CC3)cc2)C1] |
| 35a | 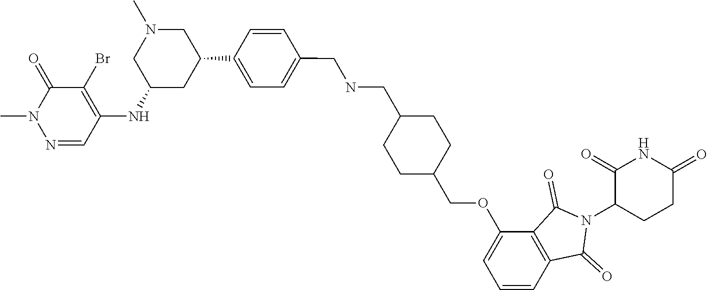 [SMILES: CN1C[C@@H](Nc2cnn(C)c(=O)c2Br)C[C@@H](c2ccc(CNCC3CCC(COc4cccc5c4C(=O)N(C4CCC(=O)NC4=O)C5=O)CC3)cc2)C1] |
| 35b | 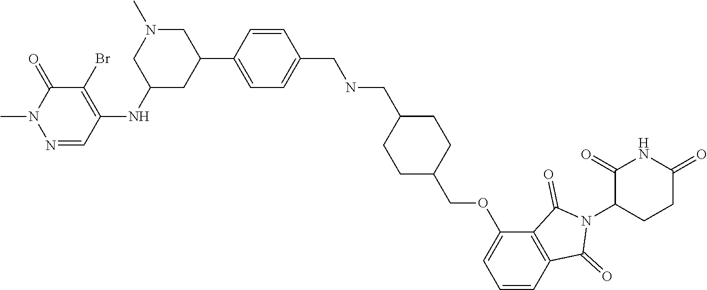 [SMILES: CN1CC(Nc2cnn(C)c(=O)c2Br)CC(c2ccc(CNCC3CCC(COc4cccc5c4C(=O)N(C4CCC(=O)NC4=O)C5=O)CC3)cc2)C1] |
| 36 | 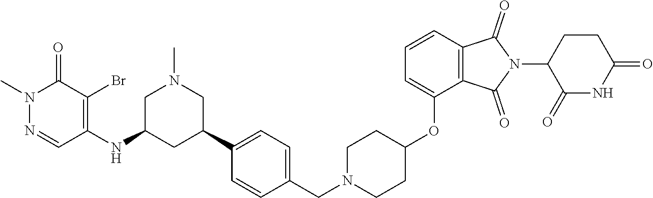 [SMILES: CN1C[C@H](Nc2cnn(C)c(=O)c2Br)C[C@H](c2ccc(CN3CCC(Oc4cccc5c4C(=O)N(C4CCC(=O)NC4=O)C5=O)CC3)cc2)C1] |
| 36a | 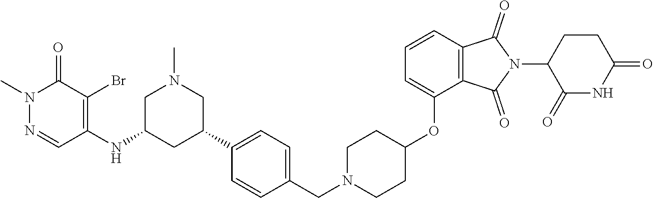 [SMILES: CN1C[C@@H](Nc2cnn(C)c(=O)c2Br)C[C@@H](c2ccc(CN3CCC(Oc4cccc5c4C(=O)N(C4CCC(=O)NC4=O)C5=O)CC3)cc2)C1] |
| 36b | 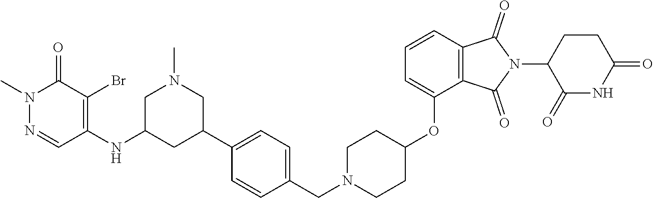 [SMILES: CN1CC(Nc2cnn(C)c(=O)c2Br)CC(c2ccc(CN3CCC(Oc4cccc5c4C(=O)N(C4CCC(=O)NC4=O)C5=O)CC3)cc2)C1] |
| 37 | 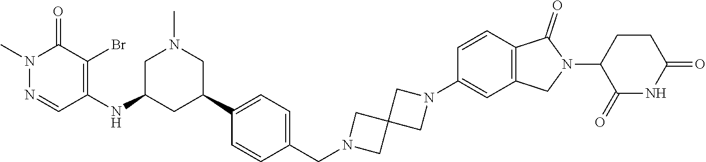 [SMILES: CN1C[C@H](Nc2cnn(C)c(=O)c2Br)C[C@H](c2ccc(CN3CC4(C3)CN(c3ccc5c(c3)CN(C3CCC(=O)NC3=O)C5=O)C4)cc2)C1] |
| 37a | 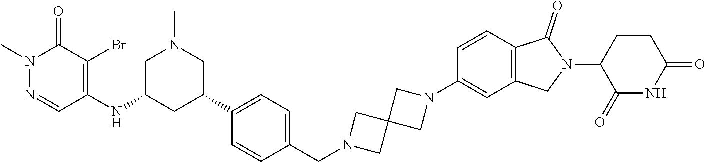 [SMILES: CN1C[C@@H](Nc2cnn(C)c(=O)c2Br)C[C@@H](c2ccc(CN3CC4(C3)CN(c3ccc5c(c3)CN(C3CCC(=O)NC3=O)C5=O)C4)cc2)C1] |
| 37b | 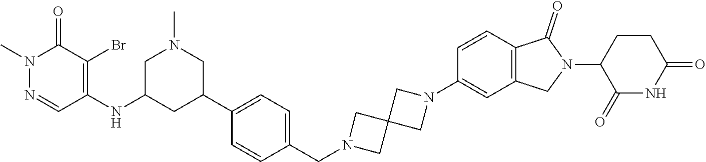 [SMILES: CN1CC(Nc2cnn(C)c(=O)c2Br)CC(c2ccc(CN3CC4(C3)CN(c3ccc5c(c3)CN(C3CCC(=O)NC3=O)C5=O)C4)cc2)C1] |
| 38 | 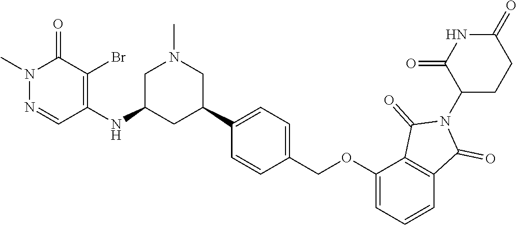 [SMILES: CN1C[C@H](Nc2cnn(C)c(=O)c2Br)C[C@H](c2ccc(COc3cccc4c3C(=O)N(C3CCC(=O)NC3=O)C4=O)cc2)C1] |
| 38a | 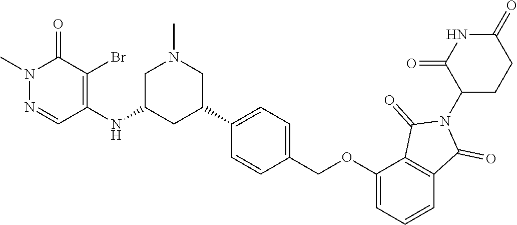 |
| 38b | 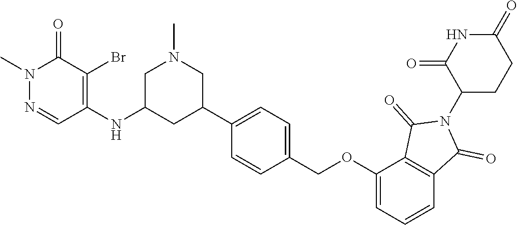 [SMILES: CN1CC(Nc2cnn(C)c(=O)c2Br)CC(c2ccc(COc3cccc4c3C(=O)N(C3CCC(=O)NC3=O)C4=O)cc2)C1] |
| 39 | 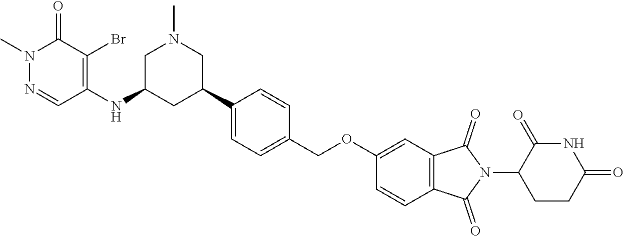 [SMILES: CN1C[C@H](Nc2cnn(C)c(=O)c2Br)C[C@H](c2ccc(COc3ccc4c(c3)C(=O)N(C3CCC(=O)NC3=O)C4=O)cc2)C1] |
| 39a | 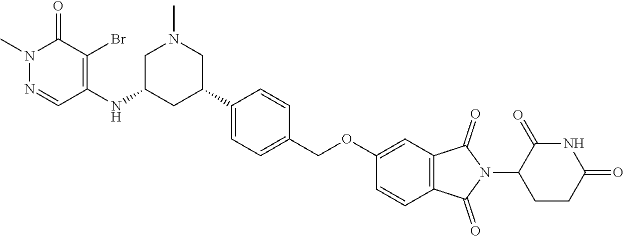 [SMILES: CN1C[C@@H](Nc2cnn(C)c(=O)c2Br)C[C@@H](c2ccc(COc3ccc4c(c3)C(=O)N(C3CCC(=O)NC3=O)C4=O)cc2)C1] |
| 39b | 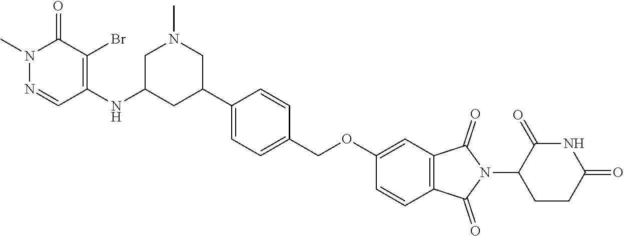 [SMILES: CN1CC(Nc2cnn(C)c(=O)c2Br)CC(c2ccc(COc3ccc4c(c3)C(=O)N(C3CCC(=O)NC3=O)C4=O)cc2)C1] |
| 40 | 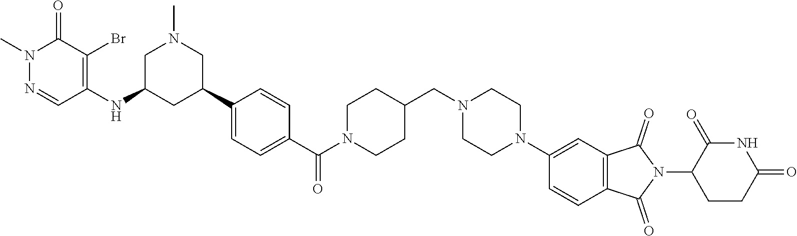 [SMILES: CN1C[C@H](Nc2cnn(C)c(=O)c2Br)C[C@H](c2ccc(C(=O)N3CCC(CN4CCN(c5ccc6c(c5)C(=O)N(C5CCC(=O)NC5=O)C6=O)CC4)CC3)cc2)C1] |
| 40a | 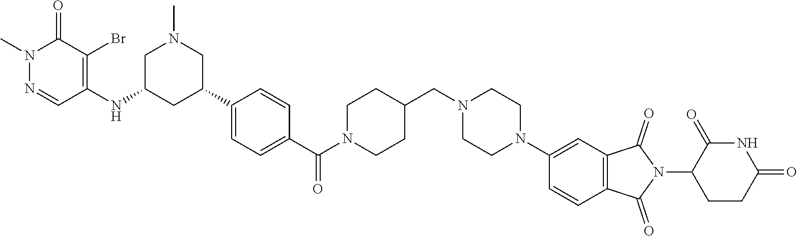 [SMILES: CN1C[C@@H](Nc2cnn(C)c(=O)c2Br)C[C@@H](c2ccc(C(=O)N3CCC(CN4CCN(c5ccc6c(c5)C(=O)N(C5CCC(=O)NC5=O)C6=O)CC4)CC3)cc2)C1] |
| 40b | 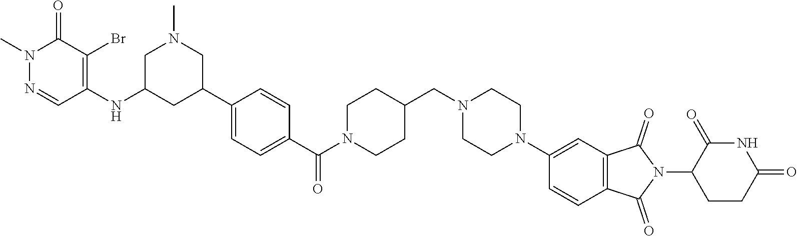 [SMILES: CN1CC(Nc2cnn(C)c(=O)c2Br)CC(c2ccc(C(=O)N3CCC(CN4CCN(c5ccc6c(c5)C(=O)N(C5CCC(=O)NC5=O)C6=O)CC4)CC3)cc2)C1] |
| 41 | 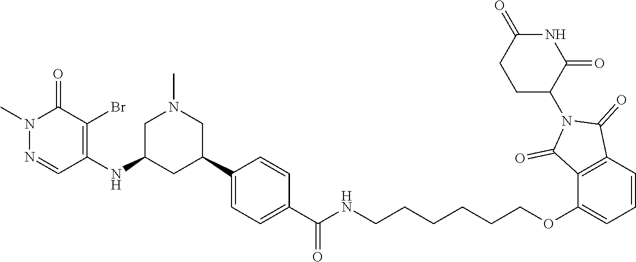 [SMILES: CN1C[C@H](Nc2cnn(C)c(=O)c2Br)C[C@H](c2ccc(C(=O)NCCCCCCOc3cccc4c3C(=O)N(C3CCC(=O)NC3=O)C4=O)cc2)C1] |
| 41a | 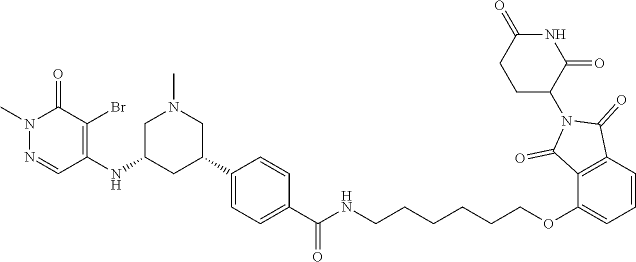 [SMILES: CN1C[C@@H](Nc2cnn(C)c(=O)c2Br)C[C@@H](c2ccc(C(=O)NCCCCCCOc3cccc4c3C(=O)N(C3CCC(=O)NC3=O)C4=O)cc2)C1] |
| 41b | 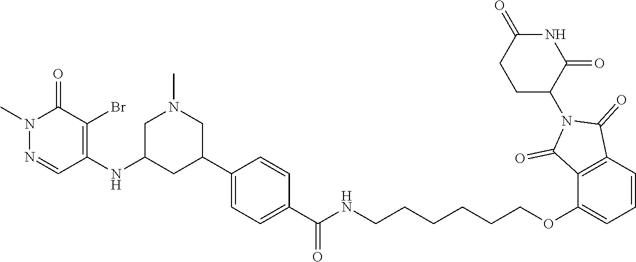 [SMILES: CN1CC(Nc2cnn(C)c(=O)c2Br)CC(c2ccc(C(=O)NCCCCCCOc3cccc4c3C(=O)N(C3CCC(=O)NC3=O)C4=O)cc2)C1] |
| 42 | 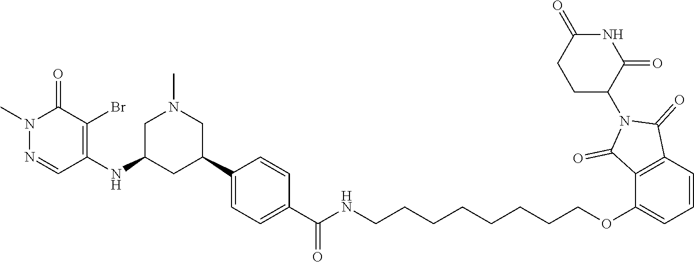 [SMILES: CN1C[C@H](Nc2cnn(C)c(=O)c2Br)C[C@H](c2ccc(C(=O)NCCCCCCCCOc3cccc4c3C(=O)N(C3CCC(=O)NC3=O)C4=O)cc2)C1] |
| 42a | 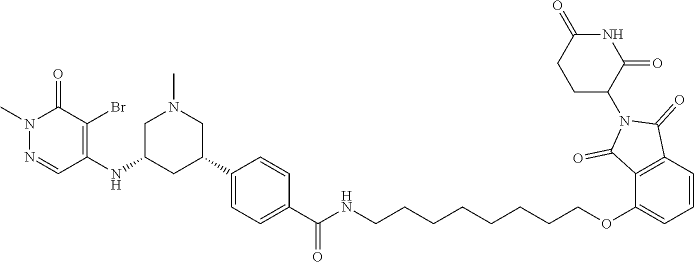 [SMILES: CN1C[C@@H](Nc2cnn(C)c(=O)c2Br)C[C@@H](c2ccc(C(=O)NCCCCCCCCOc3cccc4c3C(=O)N(C3CCC(=O)NC3=O)C4=O)cc2)C1] |
| 42b | 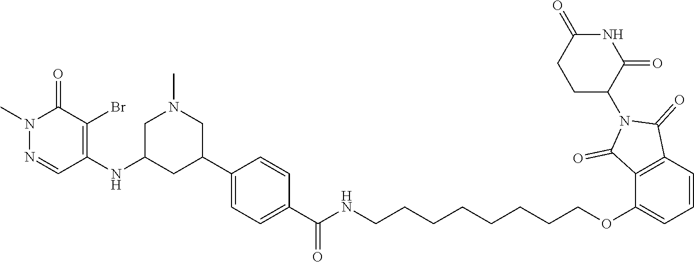 [SMILES: CN1CC(Nc2cnn(C)c(=O)c2Br)CC(c2ccc(C(=O)NCCCCCCCCOc3cccc4c3C(=O)N(C3CCC(=O)NC3=O)C4=O)cc2)C1] |
| 43 | 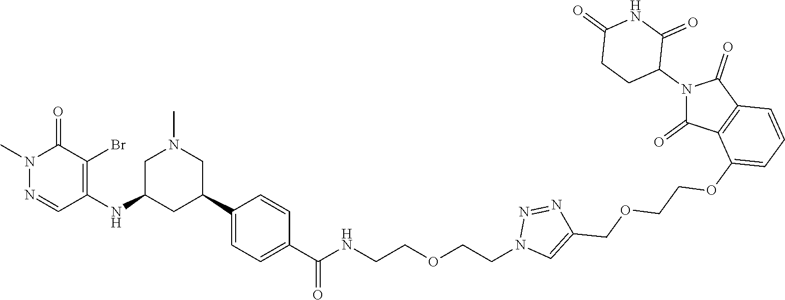 [SMILES: CN1C[C@H](Nc2cnn(C)c(=O)c2Br)C[C@H](c2ccc(C(=O)NCCOCCn3cc(COCCOc4cccc5c4C(=O)N(C4CCC(=O)NC4=O)C5=O)nn3)cc2)C1] |
| 43a | 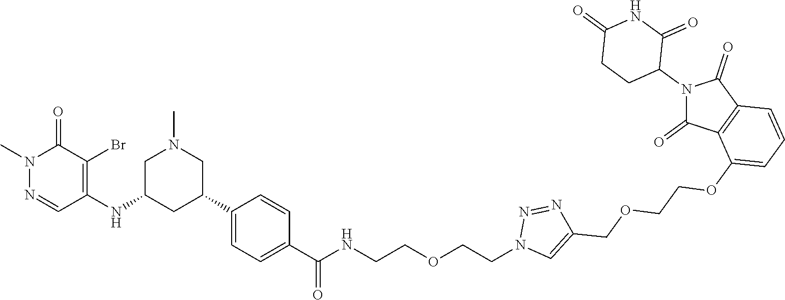 [SMILES: CN1C[C@@H](Nc2cnn(C)c(=O)c2Br)C[C@@H](c2ccc(C(=O)NCCOCCn3cc(COCCOc4cccc5c4C(=O)N(C4CCC(=O)NC4=O)C5=O)nn3)cc2)C1] |
| 43b | 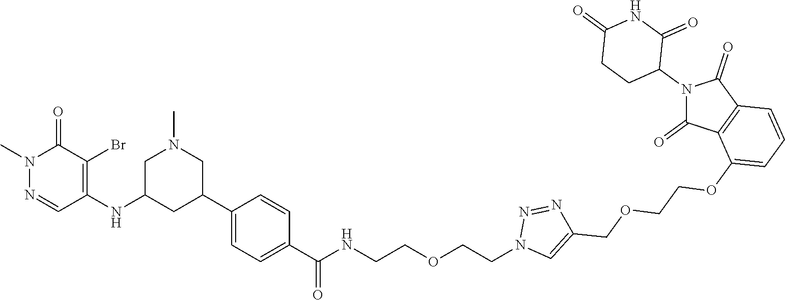 [SMILES: CN1CC(Nc2cnn(C)c(=O)c2Br)CC(c2ccc(C(=O)NCCOCCn3cc(COCCOc4cccc5c4C(=O)N(C4CCC(=O)NC4=O)C5=O)nn3)cc2)C1] |
| 44 | 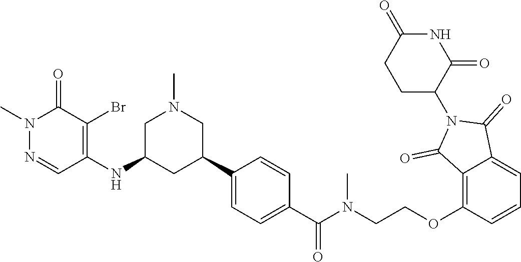 [SMILES: CN1C[C@H](Nc2cnn(C)c(=O)c2Br)C[C@H](c2ccc(C(=O)N(C)CCOc3cccc4c3C(=O)N(C3CCC(=O)NC3=O)C4=O)cc2)C1] |
| 44a | 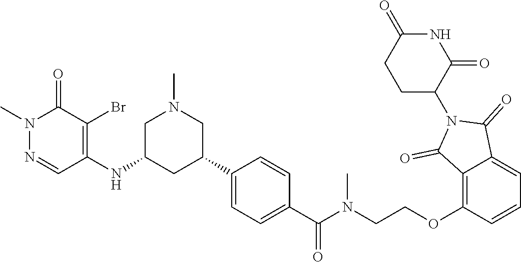 [SMILES: CN1C[C@@H](Nc2cnn(C)c(=O)c2Br)C[C@@H](c2ccc(C(=O)N(C)CCOc3cccc4c3C(=O)N(C3CCC(=O)NC3=O)C4=O)cc2)C1] |
| 44b | 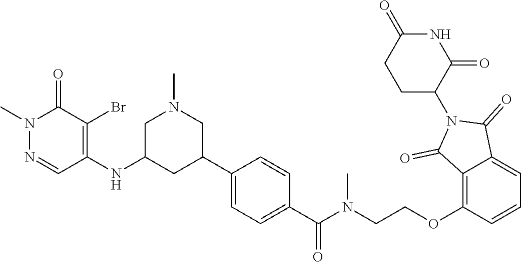 [SMILES: CN1CC(Nc2cnn(C)c(=O)c2Br)CC(c2ccc(C(=O)N(C)CCOc3cccc4c3C(=O)N(C3CCC(=O)NC3=O)C4=O)cc2)C1] |
| 45 | 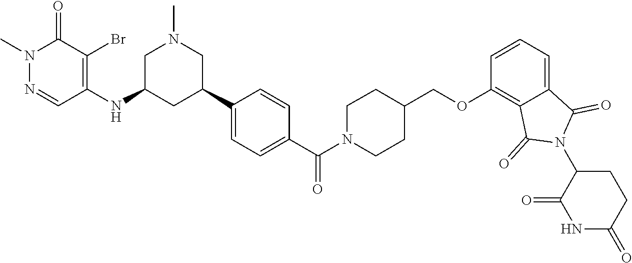 [SMILES: CN1C[C@H](Nc2cnn(C)c(=O)c2Br)C[C@H](c2ccc(C(=O)N3CCC(COc4cccc5c4C(=O)N(C4CCC(=O)NC4=O)C5=O)CC3)cc2)C1] |
| 45a | 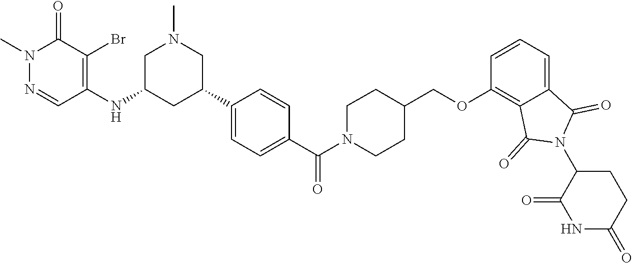 [SMILES: CN1C[C@@H](Nc2cnn(C)c(=O)c2Br)C[C@@H](c2ccc(C(=O)N3CCC(COc4cccc5c4C(=O)N(C4CCC(=O)NC4=O)C5=O)CC3)cc2)C1] |
| 45b | 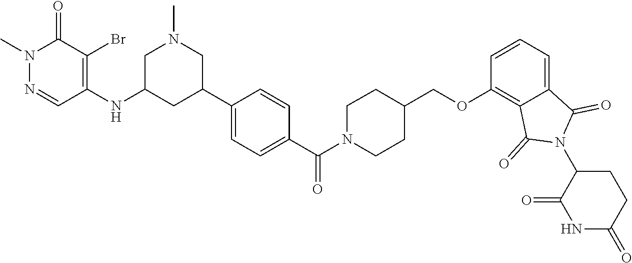 [SMILES: CN1CC(Nc2cnn(C)c(=O)c2Br)CC(c2ccc(C(=O)N3CCC(COc4cccc5c4C(=O)N(C4CCC(=O)NC4=O)C5=O)CC3)cc2)C1] |
| 46 | 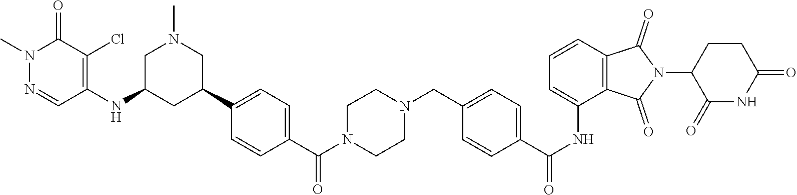 [SMILES: CN1C[C@H](Nc2cnn(C)c(=O)c2Cl)C[C@H](c2ccc(C(=O)N3CCN(Cc4ccc(C(=O)Nc5cccc6c5C(=O)N(C5CCC(=O)NC5=O)C6=O)cc4)CC3)cc2)C1] |
| 46a | 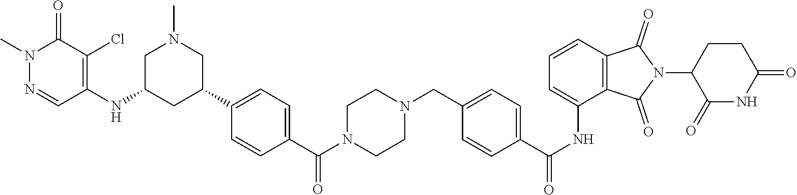 [SMILES: CN1C[C@@H](Nc2cnn(C)c(=O)c2Cl)C[C@@H](c2ccc(C(=O)N3CCN(Cc4ccc(C(=O)Nc5cccc6c5C(=O)N(C5CCC(=O)NC5=O)C6=O)cc4)CC3)cc2)C1] |
| 46b | 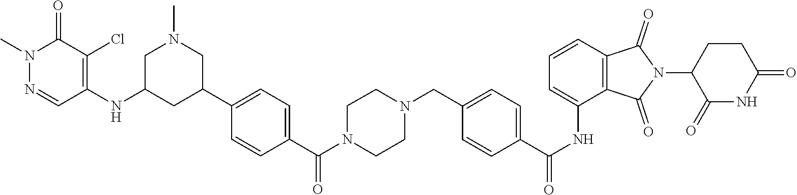 [SMILES: CN1CC(Nc2cnn(C)c(=O)c2Cl)CC(c2ccc(C(=O)N3CCN(Cc4ccc(C(=O)Nc5cccc6c5C(=O)N(C5CCC(=O)NC5=O)C6=O)cc4)CC3)cc2)C1] |
| 47 | 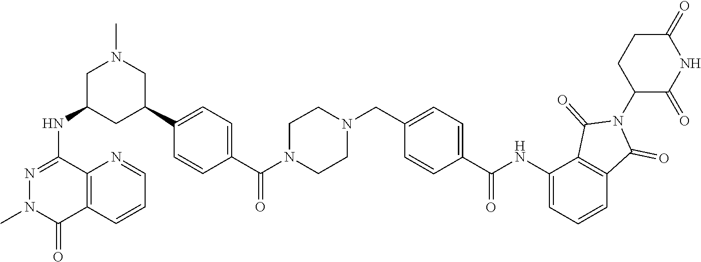 [SMILES: CN1C[C@H](Nc2nn(C)c(=O)c3cccnc23)C[C@H](c2ccc(C(=O)N3CCN(Cc4ccc(C(=O)Nc5cccc6c5C(=O)N(C5CCC(=O)NC5=O)C6=O)cc4)CC3)cc2)C1] |
| 47a | 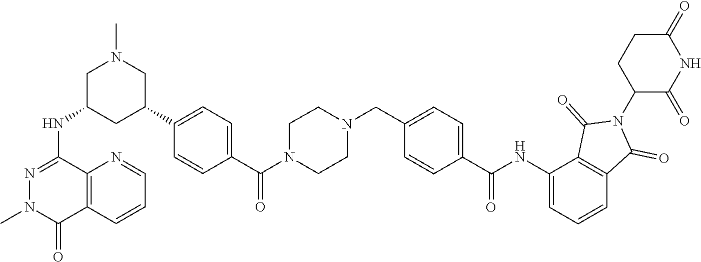 [SMILES: CN1C[C@@H](Nc2nn(C)c(=O)c3cccnc23)C[C@@H](c2ccc(C(=O)N3CCN(Cc4ccc(C(=O)Nc5cccc6c5C(=O)N(C5CCC(=O)NC5=O)C6=O)cc4)CC3)cc2)C1] |
| 47b | 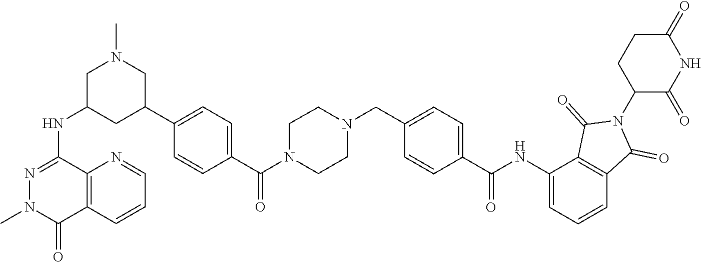 [SMILES: CN1CC(Nc2nn(C)c(=O)c3cccnc23)CC(c2ccc(C(=O)N3CCN(Cc4ccc(C(=O)Nc5cccc6c5C(=O)N(C5CCC(=O)NC5=O)C6=O)cc4)CC3)cc2)C1] |
| 48 | 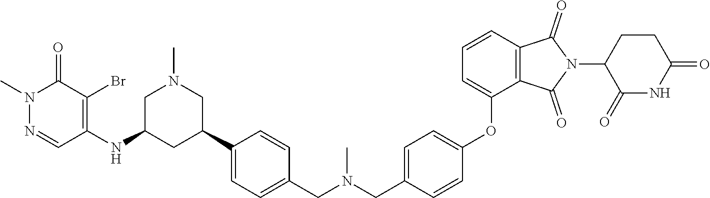 [SMILES: CN(Cc1ccc(Oc2cccc3c2C(=O)N(C2CCC(=O)NC2=O)C3=O)cc1)Cc1ccc([C@H]2C[C@@H](Nc3cnn(C)c(=O)c3Br)CN(C)C2)cc1] |
| 48a | 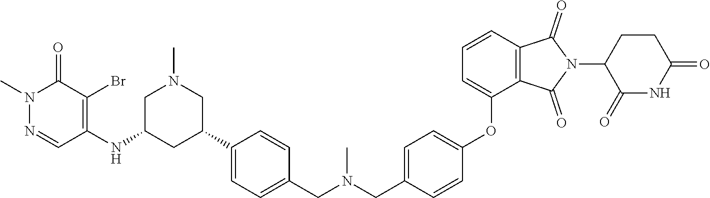 [SMILES: CN(Cc1ccc(Oc2cccc3c2C(=O)N(C2CCC(=O)NC2=O)C3=O)cc1)Cc1ccc([C@@H]2C[C@H](Nc3cnn(C)c(=O)c3Br)CN(C)C2)cc1] |
| 48b | 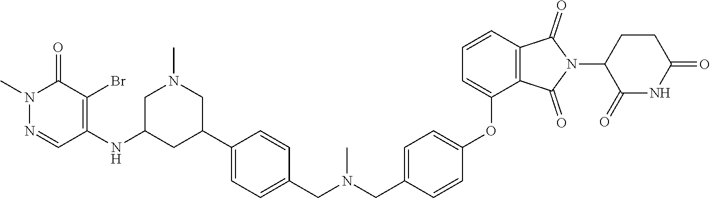 [SMILES: CN(Cc1ccc(Oc2cccc3c2C(=O)N(C2CCC(=O)NC2=O)C3=O)cc1)Cc1ccc(C2CC(Nc3cnn(C)c(=O)c3Br)CN(C)C2)cc1] |
| 49 | 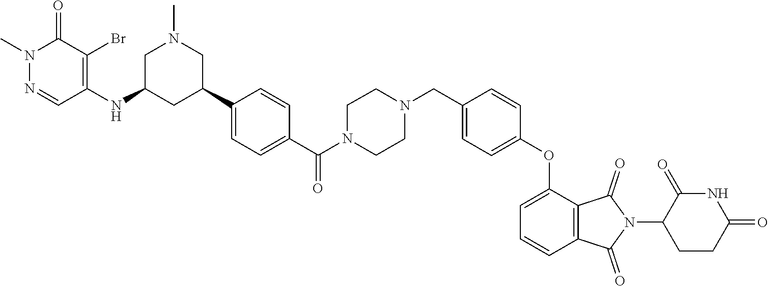 [SMILES: CN1C[C@H](Nc2cnn(C)c(=O)c2Br)C[C@H](c2ccc(C(=O)N3CCN(Cc4ccc(Oc5cccc6c5C(=O)N(C5CCC(=O)NC5=O)C6=O)cc4)CC3)cc2)C1] |
| 49a | 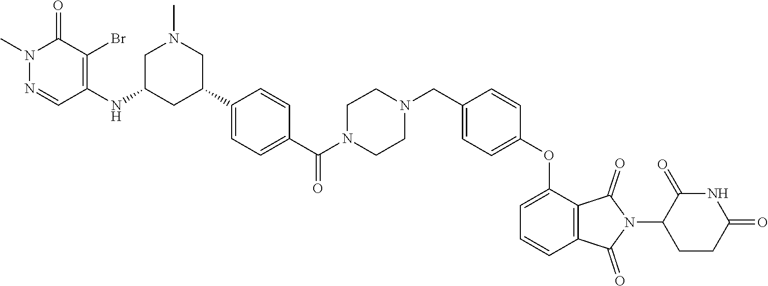 [SMILES: CN1C[C@@H](Nc2cnn(C)c(=O)c2Br)C[C@@H](c2ccc(C(=O)N3CCN(Cc4ccc(Oc5cccc6c5C(=O)N(C5CCC(=O)NC5=O)C6=O)cc4)CC3)cc2)C1] |
| 49b | 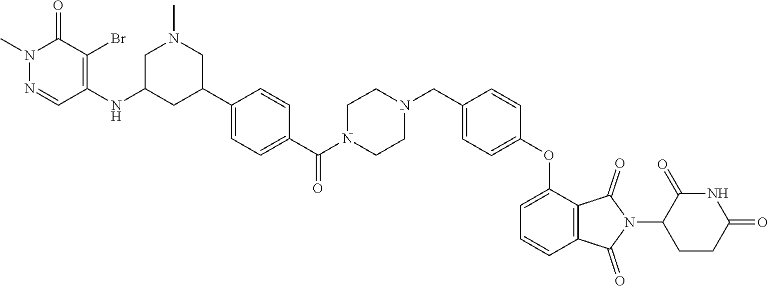 [SMILES: CN1CC(Nc2cnn(C)c(=O)c2Br)CC(c2ccc(C(=O)N3CCN(Cc4ccc(Oc5cccc6c5C(=O)N(C5CCC(=O)NC5=O)C6=O)cc4)CC3)cc2)C1] |
| 50 | 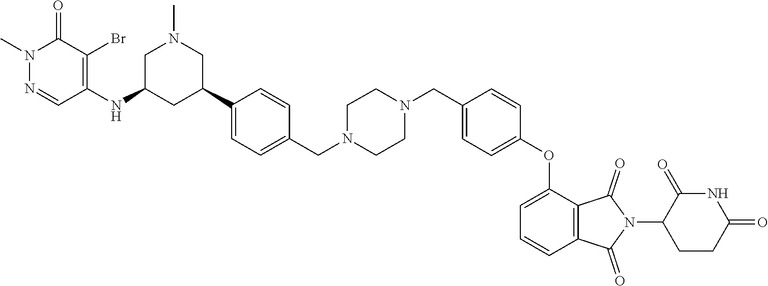 [SMILES: CN1C[C@H](Nc2cnn(C)c(=O)c2Br)C[C@H](c2ccc(CN3CCN(Cc4ccc(Oc5cccc6c5C(=O)N(C5CCC(=O)NC5=O)C6=O)cc4)CC3)cc2)C1] |
| 50a | 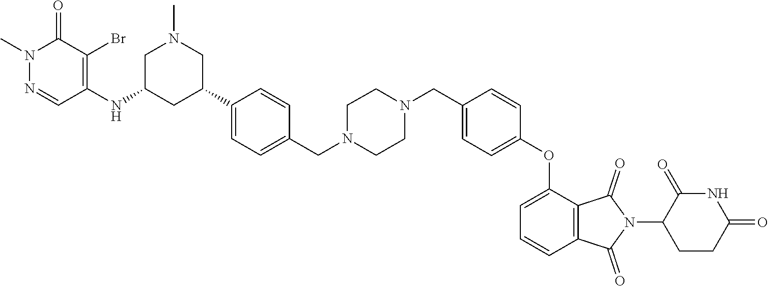 [SMILES: CN1C[C@@H](Nc2cnn(C)c(=O)c2Br)C[C@@H](c2ccc(CN3CCN(Cc4ccc(Oc5cccc6c5C(=O)N(C5CCC(=O)NC5=O)C6=O)cc4)CC3)cc2)C1] |
| 50b | 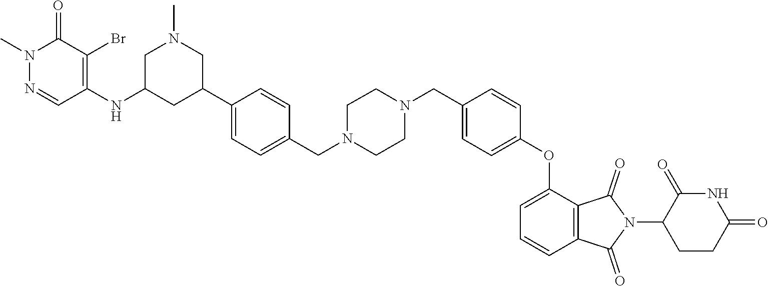 [SMILES: CN1CC(Nc2cnn(C)c(=O)c2Br)CC(c2ccc(CN3CCN(Cc4ccc(Oc5cccc6c5C(=O)N(C5CCC(=O)NC5=O)C6=O)cc4)CC3)cc2)C1] |
| 51 | 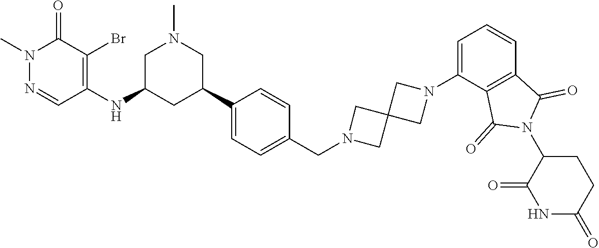 [SMILES: CN1C[C@H](Nc2cnn(C)c(=O)c2Br)C[C@H](c2ccc(CN3CC4(C3)CN(c3cccc5c3C(=O)N(C3CCC(=O)NC3=O)C5=O)C4)cc2)C1] |
| 51a | 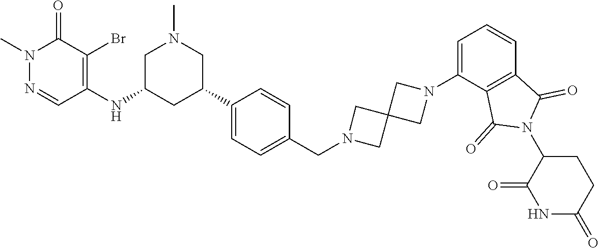 [SMILES: CN1C[C@@H](Nc2cnn(C)c(=O)c2Br)C[C@@H](c2ccc(CN3CC4(C3)CN(c3cccc5c3C(=O)N(C3CCC(=O)NC3=O)C5=O)C4)cc2)C1] |
| 51b | 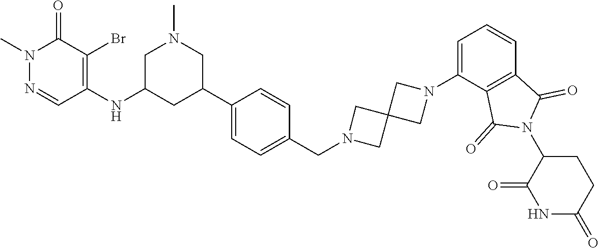 [SMILES: CN1CC(Nc2cnn(C)c(=O)c2Br)CC(c2ccc(CN3CC4(C3)CN(c3cccc5c3C(=O)N(C3CCC(=O)NC3=O)C5=O)C4)cc2)C1] |
| 52 | 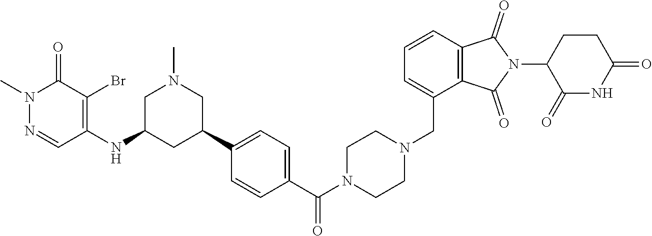 [SMILES: CN1C[C@H](Nc2cnn(C)c(=O)c2Br)C[C@H](c2ccc(C(=O)N3CCN(Cc4cccc5c4C(=O)N(C4CCC(=O)NC4=O)C5=O)CC3)cc2)C1] |
| 52a | 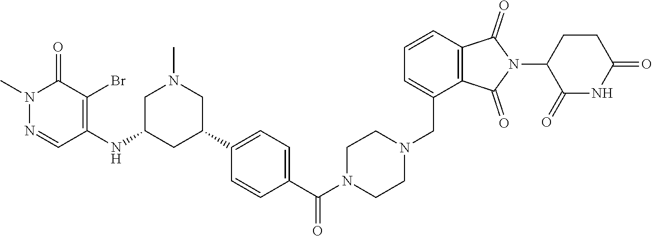 [SMILES: CN1C[C@@H](Nc2cnn(C)c(=O)c2Br)C[C@@H](c2ccc(C(=O)N3CCN(Cc4cccc5c4C(=O)N(C4CCC(=O)NC4=O)C5=O)CC3)cc2)C1] |
| 52b | 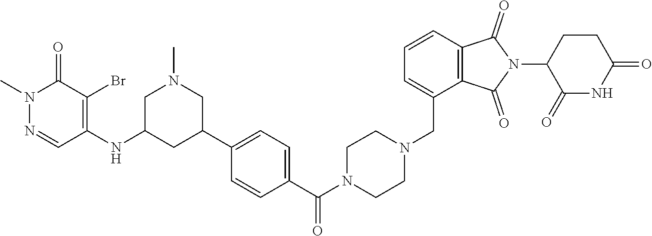 [SMILES: CN1CC(Nc2cnn(C)c(=O)c2Br)CC(c2ccc(C(=O)N3CCN(Cc4cccc5c4C(=O)N(C4CCC(=O)NC4=O)C5=O)CC3)cc2)C1] |
| 53 | 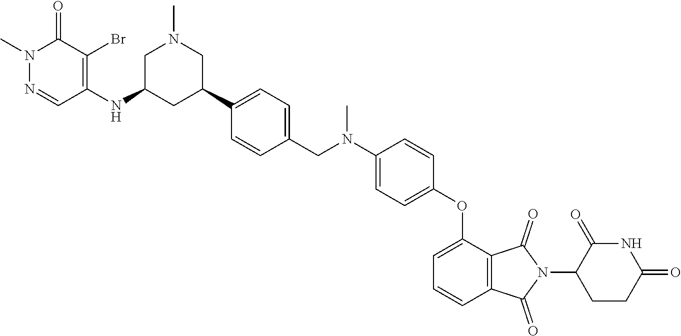 [SMILES: CN1C[C@H](Nc2cnn(C)c(=O)c2Br)C[C@H](c2ccc(CN(C)c3ccc(Oc4cccc5c4C(=O)N(C4CCC(=O)NC4=O)C5=O)cc3)cc2)C1] |
| 53a | 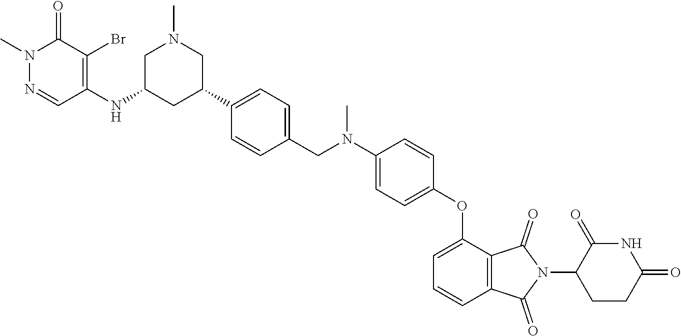 [SMILES: CN1C[C@@H](Nc2cnn(C)c(=O)c2Br)C[C@@H](c2ccc(CN(C)c3ccc(Oc4cccc5c4C(=O)N(C4CCC(=O)NC4=O)C5=O)cc3)cc2)C1] |
| 53b | 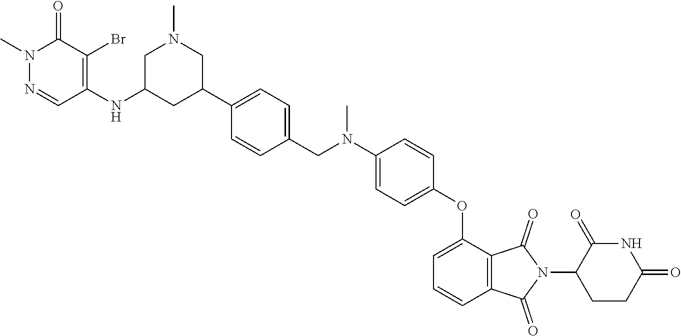 [SMILES: CN1CC(Nc2cnn(C)c(=O)c2Br)CC(c2ccc(CN(C)c3ccc(Oc4cccc5c4C(=O)N(C4CCC(=O)NC4=O)C5=O)cc3)cc2)C1] |
| 54 | 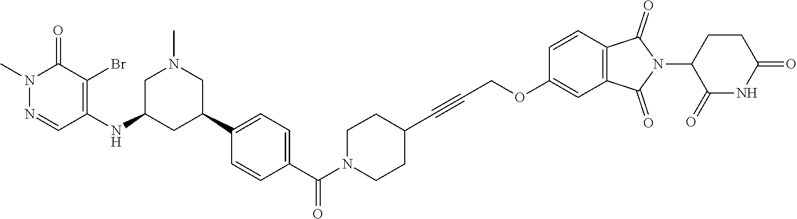 [SMILES: CN1C[C@H](Nc2cnn(C)c(=O)c2Br)C[C@H](c2ccc(C(=O)N3CCC(C#CCOc4ccc5c(c4)C(=O)N(C4CCC(=O)NC4=O)C5=O)CC3)cc2)C1] |
| 54a | 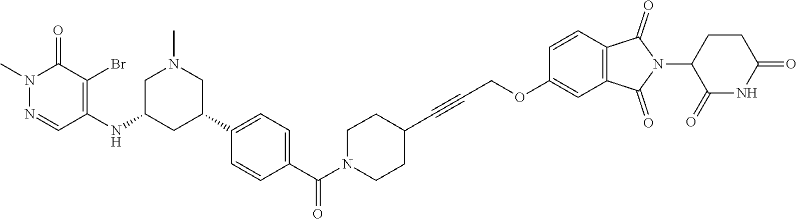 [SMILES: CN1C[C@@H](Nc2cnn(C)c(=O)c2Br)C[C@@H](c2ccc(C(=O)N3CCC(C#CCOc4ccc5c(c4)C(=O)N(C4CCC(=O)NC4=O)C5=O)CC3)cc2)C1] |
| 54b | 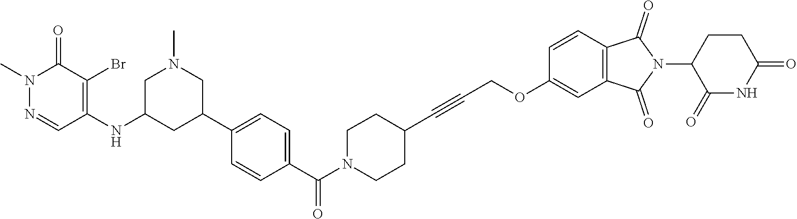 [SMILES: CN1CC(Nc2cnn(C)c(=O)c2Br)CC(c2ccc(C(=O)N3CCC(C#CCOc4ccc5c(c4)C(=O)N(C4CCC(=O)NC4=O)C5=O)CC3)cc2)C1] |
| 55 | 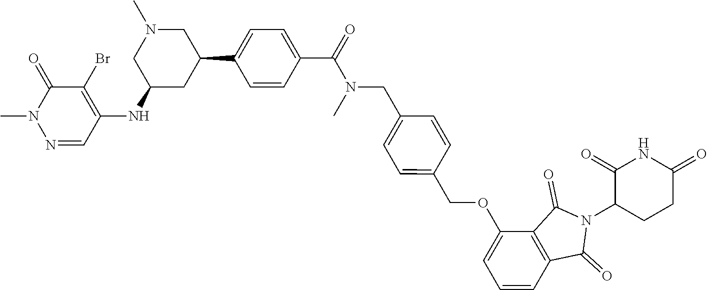 [SMILES: CN1C[C@H](Nc2cnn(C)c(=O)c2Br)C[C@H](c2ccc(C(=O)N(C)Cc3ccc(COc4cccc5c4C(=O)N(C4CCC(=O)NC4=O)C5=O)cc3)cc2)C1] |
| 55a | 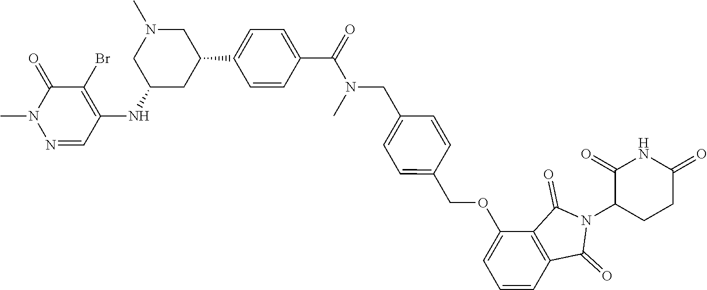 [SMILES: CN1C[C@@H](Nc2cnn(C)c(=O)c2Br)C[C@@H](c2ccc(C(=O)N(C)Cc3ccc(COc4cccc5c4C(=O)N(C4CCC(=O)NC4=O)C5=O)cc3)cc2)C1] |
| 55b | 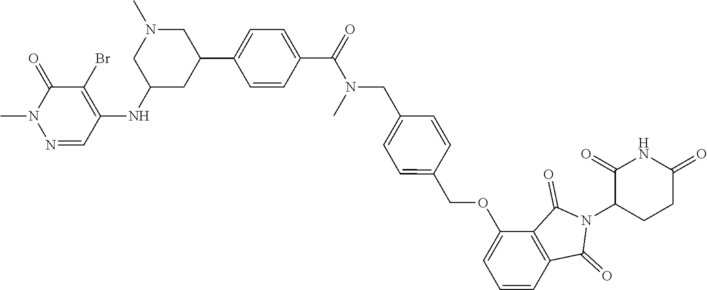 [SMILES: CN1CC(Nc2cnn(C)c(=O)c2Br)CC(c2ccc(C(=O)N(C)Cc3ccc(COc4cccc5c4C(=O)N(C4CCC(=O)NC4=O)C5=O)cc3)cc2)C1] |
| 56 | 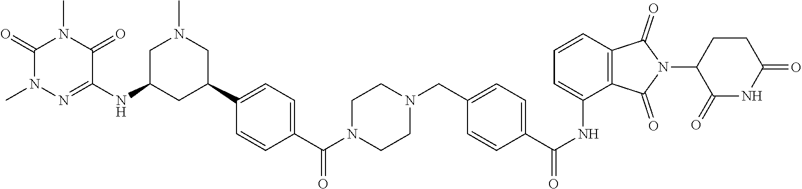 [SMILES: CN1C[C@H](Nc2nn(C)c(=O)n(C)c2=O)C[C@H](c2ccc(C(=O)N3CCN(Cc4ccc(C(=O)Nc5cccc6c5C(=O)N(C5CCC(=O)NC5=O)C6=O)cc4)CC3)cc2)C1] |
| 56a | 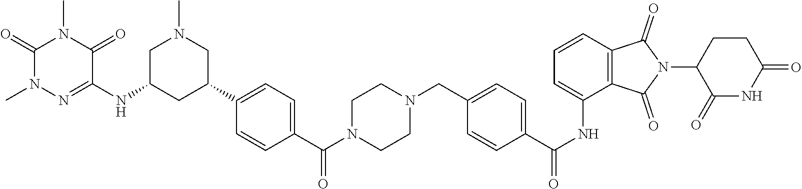 [SMILES: CN1C[C@@H](Nc2nn(C)c(=O)n(C)c2=O)C[C@@H](c2ccc(C(=O)N3CCN(Cc4ccc(C(=O)Nc5cccc6c5C(=O)N(C5CCC(=O)NC5=O)C6=O)cc4)CC3)cc2)C1] |
| 56b | 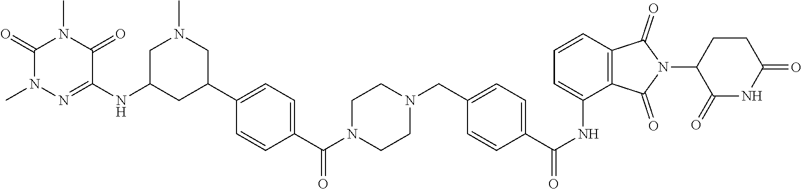 [SMILES: CN1CC(Nc2nn(C)c(=O)n(C)c2=O)CC(c2ccc(C(=O)N3CCN(Cc4ccc(C(=O)Nc5cccc6c5C(=O)N(C5CCC(=O)NC5=O)C6=O)cc4)CC3)cc2)C1] |
| 57 | 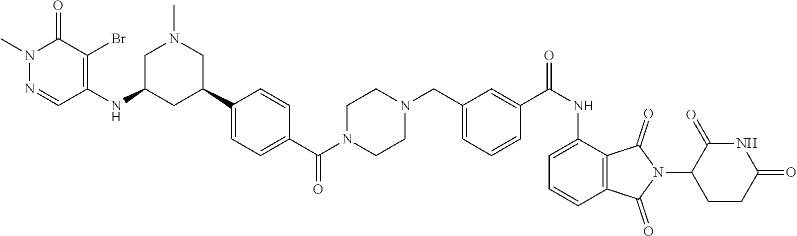 [SMILES: CN1C[C@H](Nc2cnn(C)c(=O)c2Br)C[C@H](c2ccc(C(=O)N3CCN(Cc4cccc(C(=O)Nc5cccc6c5C(=O)N(C5CCC(=O)NC5=O)C6=O)c4)CC3)cc2)C1] |
| 57a | 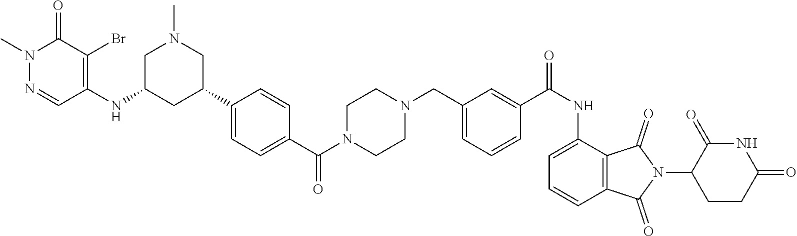 [SMILES: CN1C[C@@H](Nc2cnn(C)c(=O)c2Br)C[C@@H](c2ccc(C(=O)N3CCN(Cc4cccc(C(=O)Nc5cccc6c5C(=O)N(C5CCC(=O)NC5=O)C6=O)c4)CC3)cc2)C1] |
| 57b | 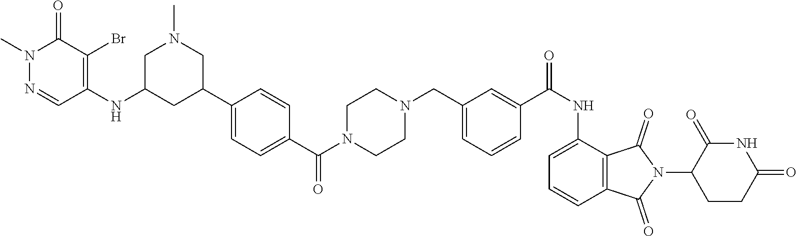 [SMILES: CN1CC(Nc2cnn(C)c(=O)c2Br)CC(c2ccc(C(=O)N3CCN(Cc4cccc(C(=O)Nc5cccc6c5C(=O)N(C5CCC(=O)NC5=O)C6=O)c4)CC3)cc2)C1] |
| 58 | 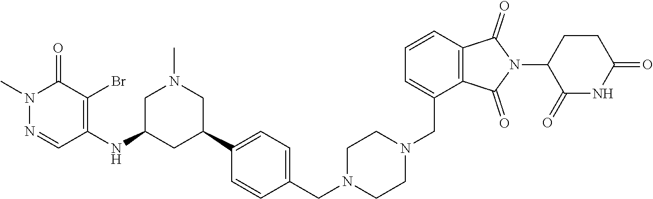 [SMILES: CN1C[C@H](Nc2cnn(C)c(=O)c2Br)C[C@H](c2ccc(CN3CCN(Cc4cccc5c4C(=O)N(C4CCC(=O)NC4=O)C5=O)CC3)cc2)C1] |
| 58a | 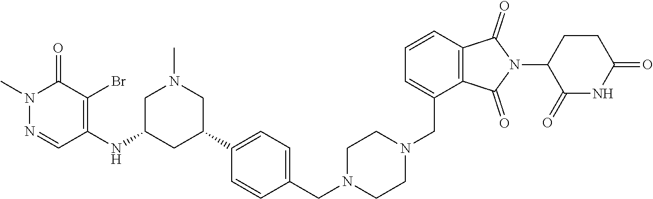 [SMILES: CN1C[C@@H](Nc2cnn(C)c(=O)c2Br)C[C@@H](c2ccc(CN3CCN(Cc4cccc5c4C(=O)N(C4CCC(=O)NC4=O)C5=O)CC3)cc2)C1] |
| 58b | 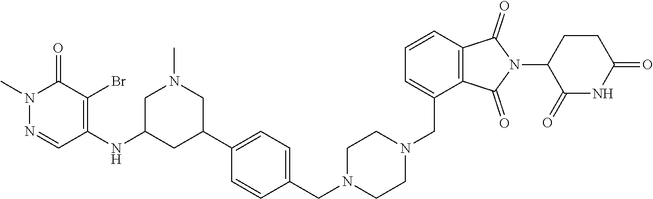 [SMILES: CN1CC(Nc2cnn(C)c(=O)c2Br)CC(c2ccc(CN3CCN(Cc4cccc5c4C(=O)N(C4CCC(=O)NC4=O)C5=O)CC3)cc2)C1] |
| 59 | 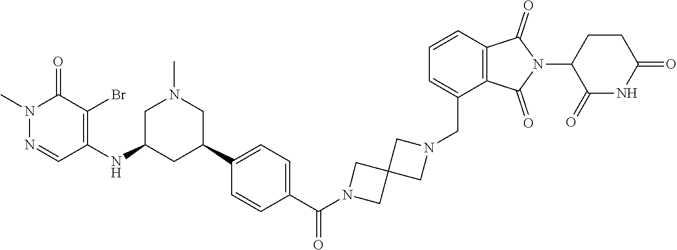 [SMILES: CN1C[C@H](Nc2cnn(C)c(=O)c2Br)C[C@H](c2ccc(C(=O)N3CC4(CN(Cc5cccc6c5C(=O)N(C5CCC(=O)NC5=O)C6=O)C4)C3)cc2)C1] |
| 59a | 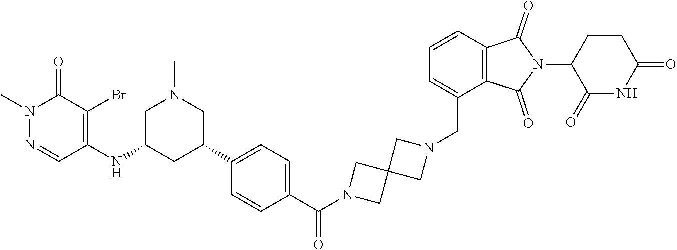 [SMILES: CN1C[C@@H](Nc2cnn(C)c(=O)c2Br)C[C@@H](c2ccc(C(=O)N3CC4(CN(Cc5cccc6c5C(=O)N(C5CCC(=O)NC5=O)C6=O)C4)C3)cc2)C1] |
| 59b | 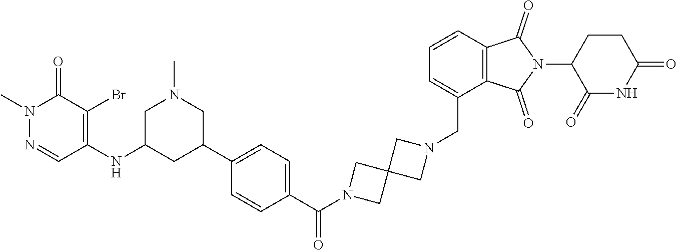 [SMILES: CN1CC(Nc2cnn(C)c(=O)c2Br)CC(c2ccc(C(=O)N3CC4(CN(Cc5cccc6c5C(=O)N(C5CCC(=O)NC5=O)C6=O)C4)C3)cc2)C1] |
| 60 | 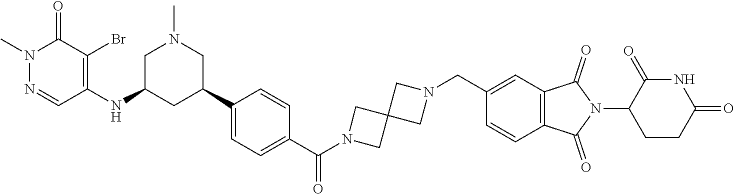 [SMILES: CN1C[C@H](Nc2cnn(C)c(=O)c2Br)C[C@H](c2ccc(C(=O)N3CC4(CN(Cc5ccc6c(c5)C(=O)N(C5CCC(=O)NC5=O)C6=O)C4)C3)cc2)C1] |
| 60a | 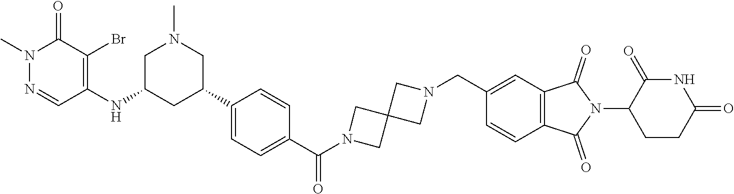 [SMILES: CN1C[C@@H](Nc2cnn(C)c(=O)c2Br)C[C@@H](c2ccc(C(=O)N3CC4(CN(Cc5ccc6c(c5)C(=O)N(C5CCC(=O)NC5=O)C6=O)C4)C3)cc2)C1] |
| 60b | 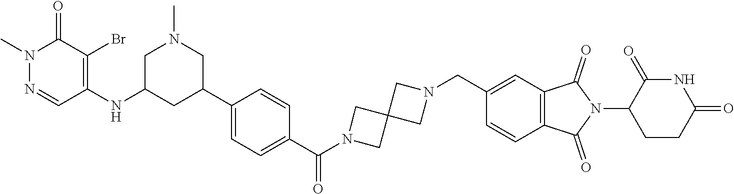 [SMILES: CN1CC(Nc2cnn(C)c(=O)c2Br)CC(c2ccc(C(=O)N3CC4(CN(Cc5ccc6c(c5)C(=O)N(C5CCC(=O)NC5=O)C6=O)C4)C3)cc2)C1] |
| 61 | 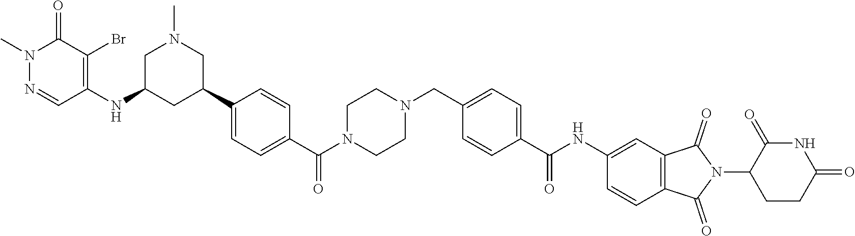 [SMILES: CN1C[C@H](Nc2cnn(C)c(=O)c2Br)C[C@H](c2ccc(C(=O)N3CCN(Cc4ccc(C(=O)Nc5ccc6c(c5)C(=O)N(C5CCC(=O)NC5=O)C6=O)cc4)CC3)cc2)C1] |
| 61a | 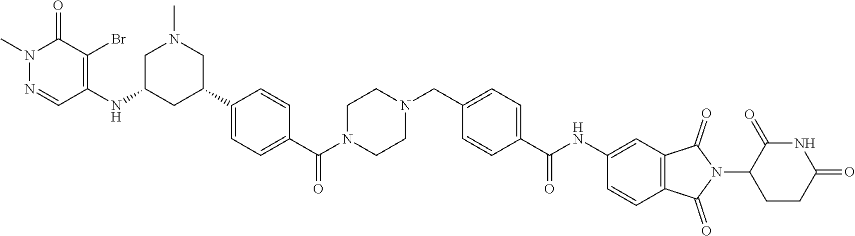 [SMILES: CN1C[C@@H](Nc2cnn(C)c(=O)c2Br)C[C@@H](c2ccc(C(=O)N3CCN(Cc4ccc(C(=O)Nc5ccc6c(c5)C(=O)N(C5CCC(=O)NC5=O)C6=O)cc4)CC3)cc2)C1] |
| 61b | 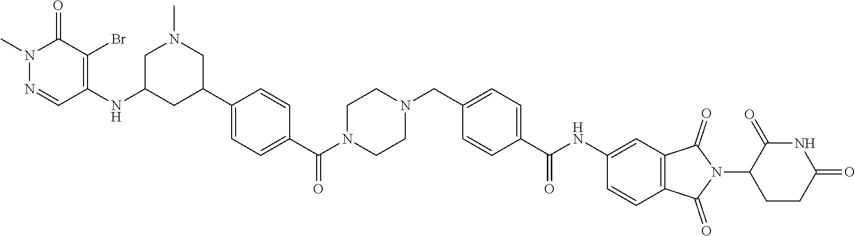 [SMILES: CN1CC(Nc2cnn(C)c(=O)c2Br)CC(c2ccc(C(=O)N3CCN(Cc4ccc(C(=O)Nc5ccc6c(c5)C(=O)N(C5CCC(=O)NC5=O)C6=O)cc4)CC3)cc2)C1] |
| 62 | 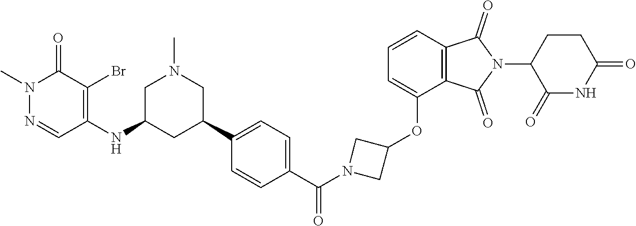 [SMILES: CN1C[C@H](Nc2cnn(C)c(=O)c2Br)C[C@H](c2ccc(C(=O)N3CC(Oc4cccc5c4C(=O)N(C4CCC(=O)NC4=O)C5=O)C3)cc2)C1] |
| 62a | 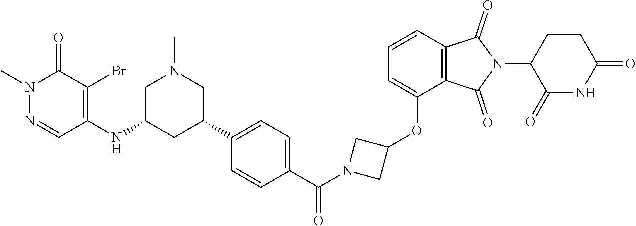 [SMILES: CN1C[C@@H](Nc2cnn(C)c(=O)c2Br)C[C@@H](c2ccc(C(=O)N3CC(Oc4cccc5c4C(=O)N(C4CCC(=O)NC4=O)C5=O)C3)cc2)C1] |
| 62b | 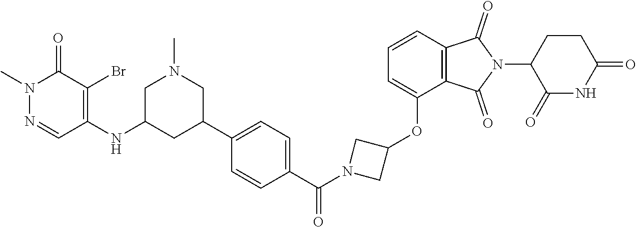 [SMILES: CN1CC(Nc2cnn(C)c(=O)c2Br)CC(c2ccc(C(=O)N3CC(Oc4cccc5c4C(=O)N(C4CCC(=O)NC4=O)C5=O)C3)cc2)C1] |
| 63 | 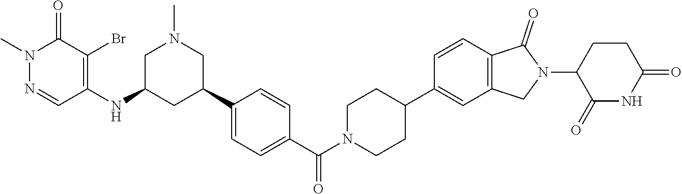 [SMILES: CN1C[C@H](Nc2cnn(C)c(=O)c2Br)C[C@H](c2ccc(C(=O)N3CCC(c4ccc5c(c4)CN(C4CCC(=O)NC4=O)C5=O)CC3)cc2)C1] |
| 63a | 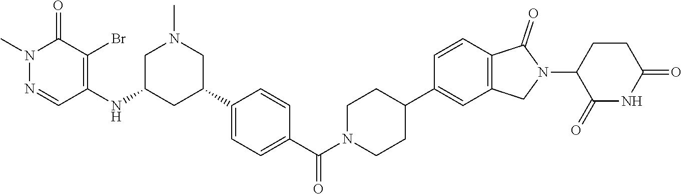 [SMILES: CN1C[C@@H](Nc2cnn(C)c(=O)c2Br)C[C@@H](c2ccc(C(=O)N3CCC(c4ccc5c(c4)CN(C4CCC(=O)NC4=O)C5=O)CC3)cc2)C1] |
| 63b | 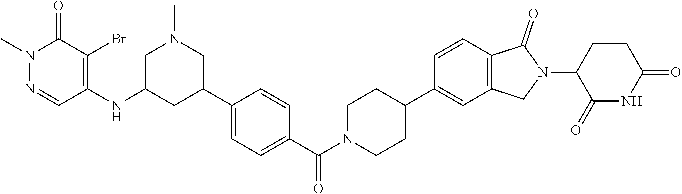 [SMILES: CN1CC(Nc2cnn(C)c(=O)c2Br)CC(c2ccc(C(=O)N3CCC(c4ccc5c(c4)CN(C4CCC(=O)NC4=O)C5=O)CC3)cc2)C1] |
| 64 | 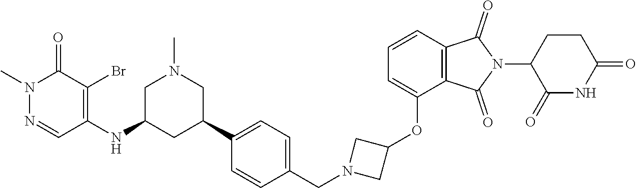 [SMILES: CN1C[C@H](Nc2cnn(C)c(=O)c2Br)C[C@H](c2ccc(CN3CC(Oc4cccc5c4C(=O)N(C4CCC(=O)NC4=O)C5=O)C3)cc2)C1] |
| 64a | 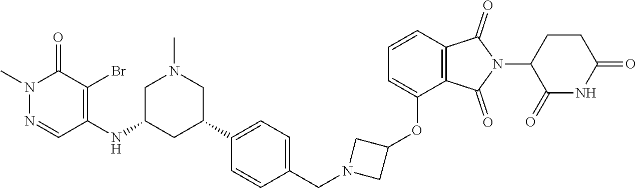 [SMILES: CN1C[C@@H](Nc2cnn(C)c(=O)c2Br)C[C@@H](c2ccc(CN3CC(Oc4cccc5c4C(=O)N(C4CCC(=O)NC4=O)C5=O)C3)cc2)C1] |
| 64b | 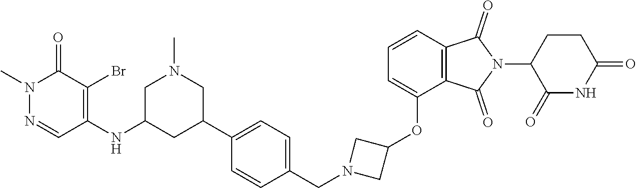 [SMILES: CN1CC(Nc2cnn(C)c(=O)c2Br)CC(c2ccc(CN3CC(Oc4cccc5c4C(=O)N(C4CCC(=O)NC4=O)C5=O)C3)cc2)C1] |
| 65 | 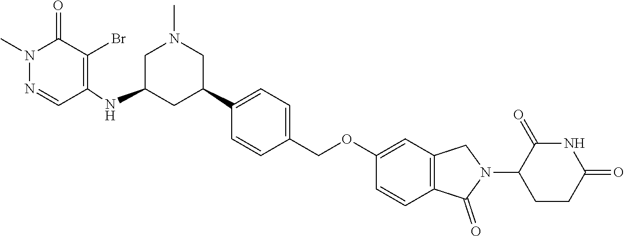 [SMILES: CN1C[C@H](Nc2cnn(C)c(=O)c2Br)C[C@H](c2ccc(COc3ccc4c(c3)CN(C3CCC(=O)NC3=O)C4=O)cc2)C1] |
| 65a | 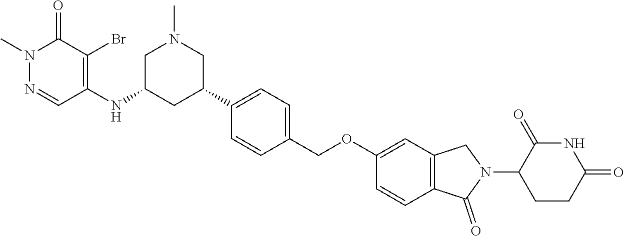 [SMILES: CN1C[C@@H](Nc2cnn(C)c(=O)c2Br)C[C@@H](c2ccc(COc3ccc4c(c3)CN(C3CCC(=O)NC3=O)C4=O)cc2)C1] |
| 65b | 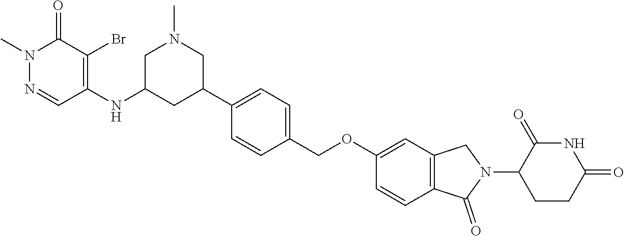 [SMILES: CN1CC(Nc2cnn(C)c(=O)c2Br)CC(c2ccc(COc3ccc4c(c3)CN(C3CCC(=O)NC3=O)C4=O)cc2)C1] |
| 66 | 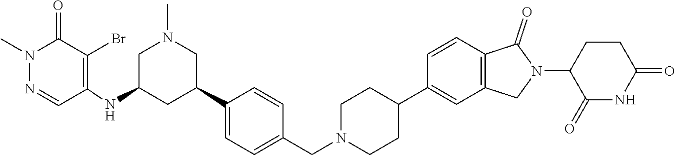 [SMILES: CN1C[C@H](Nc2cnn(C)c(=O)c2Br)C[C@H](c2ccc(CN3CCC(c4ccc5c(c4)CN(C4CCC(=O)NC4=O)C5=O)CC3)cc2)C1] |
| 66a | 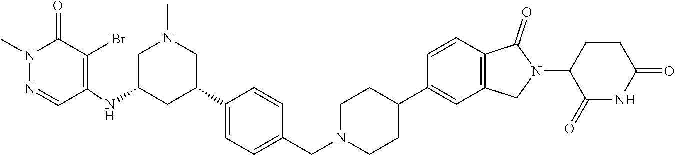 [SMILES: CN1C[C@@H](Nc2cnn(C)c(=O)c2Br)C[C@@H](c2ccc(CN3CCC(c4ccc5c(c4)CN(C4CCC(=O)NC4=O)C5=O)CC3)cc2)C1] |
| 66b | 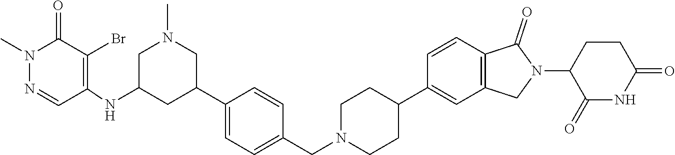 [SMILES: CN1CC(Nc2cnn(C)c(=O)c2Br)CC(c2ccc(CN3CCC(c4ccc5c(c4)CN(C4CCC(=O)NC4=O)C5=O)CC3)cc2)C1] |
| 67 | 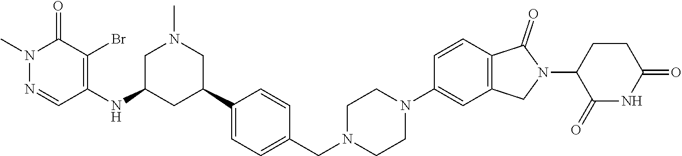 [SMILES: CN1C[C@H](Nc2cnn(C)c(=O)c2Br)C[C@H](c2ccc(CN3CCN(c4ccc5c(c4)CN(C4CCC(=O)NC4=O)C5=O)CC3)cc2)C1] |
| 67a | 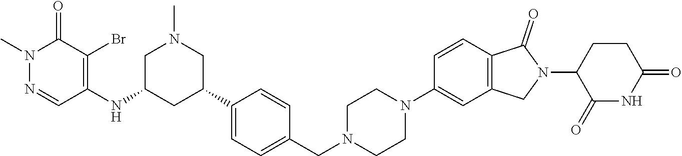 [SMILES: CN1C[C@@H](Nc2cnn(C)c(=O)c2Br)C[C@@H](c2ccc(CN3CCN(c4ccc5c(c4)CN(C4CCC(=O)NC4=O)C5=O)CC3)cc2)C1] |
| 67b | 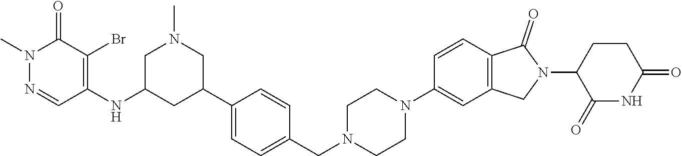 [SMILES: CN1CC(Nc2cnn(C)c(=O)c2Br)CC(c2ccc(CN3CCN(c4ccc5c(c4)CN(C4CCC(=O)NC4=O)C5=O)CC3)cc2)C1] |
| 68 | 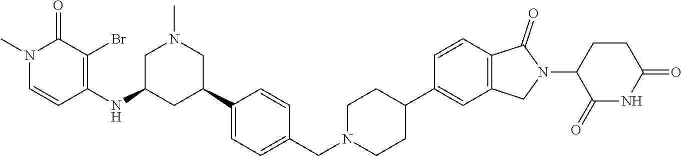 [SMILES: CN1C[C@H](Nc2ccn(C)c(=O)c2Br)C[C@H](c2ccc(CN3CCC(c4ccc5c(c4)CN(C4CCC(=O)NC4=O)C5=O)CC3)cc2)C1] |
| 68a | 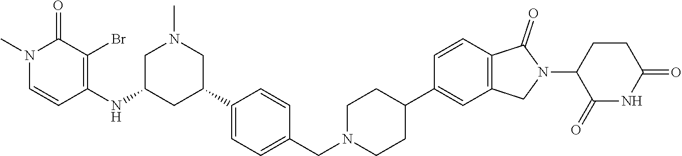 [SMILES: CN1C[C@@H](Nc2ccn(C)c(=O)c2Br)C[C@@H](c2ccc(CN3CCC(c4ccc5c(c4)CN(C4CCC(=O)NC4=O)C5=O)CC3)cc2)C1] |
| 68b | 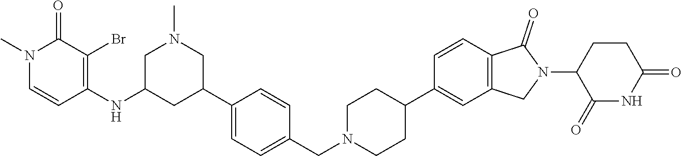 [SMILES: CN1CC(Nc2ccn(C)c(=O)c2Br)CC(c2ccc(CN3CCC(c4ccc5c(c4)CN(C4CCC(=O)NC4=O)C5=O)CC3)cc2)C1] |
| 69 | 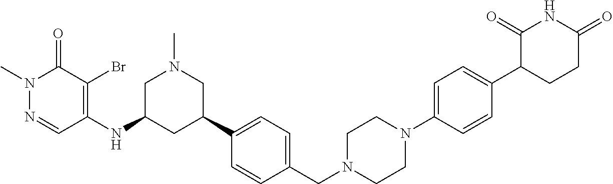 [SMILES: CN1C[C@H](Nc2cnn(C)c(=O)c2Br)C[C@H](c2ccc(CN3CCN(c4ccc(C5CCC(=O)NC5=O)cc4)CC3)cc2)C1] |
| 69a | 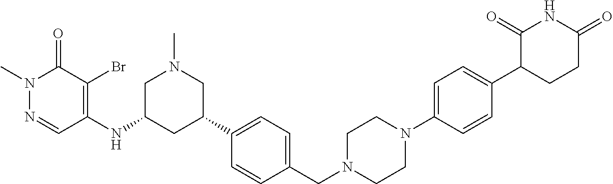 [SMILES: CN1C[C@@H](Nc2cnn(C)c(=O)c2Br)C[C@@H](c2ccc(CN3CCN(c4ccc(C5CCC(=O)NC5=O)cc4)CC3)cc2)C1] |
| 69b | 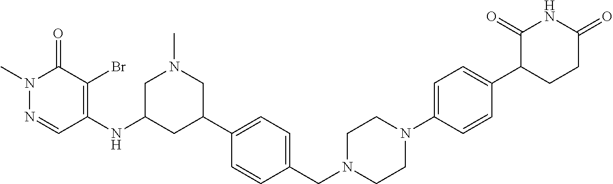 [SMILES: CN1CC(Nc2cnn(C)c(=O)c2Br)CC(c2ccc(CN3CCN(c4ccc(C5CCC(=O)NC5=O)cc4)CC3)cc2)C1] |
| 70 | 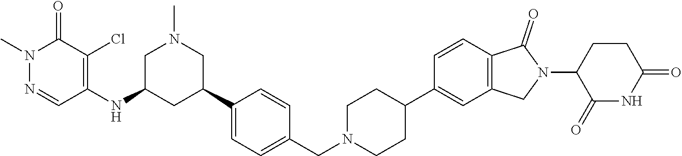 [SMILES: CN1C[C@H](Nc2cnn(C)c(=O)c2Cl)C[C@H](c2ccc(CN3CCC(c4ccc5c(c4)CN(C4CCC(=O)NC4=O)C5=O)CC3)cc2)C1] |
| 70a | 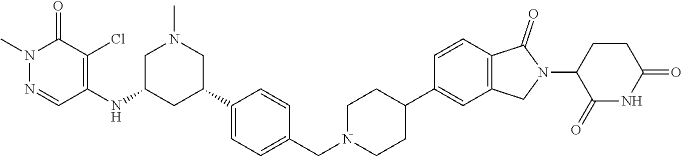 [SMILES: CN1C[C@@H](Nc2cnn(C)c(=O)c2Cl)C[C@@H](c2ccc(CN3CCC(c4ccc5c(c4)CN(C4CCC(=O)NC4=O)C5=O)CC3)cc2)C1] |
| 70b | 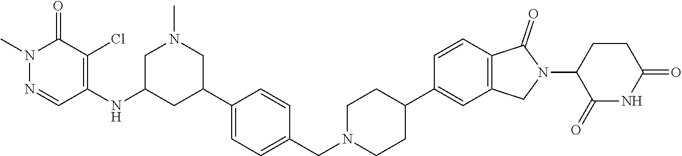 [SMILES: CN1CC(Nc2cnn(C)c(=O)c2Cl)CC(c2ccc(CN3CCC(c4ccc5c(c4)CN(C4CCC(=O)NC4=O)C5=O)CC3)cc2)C1] |
| 71 | 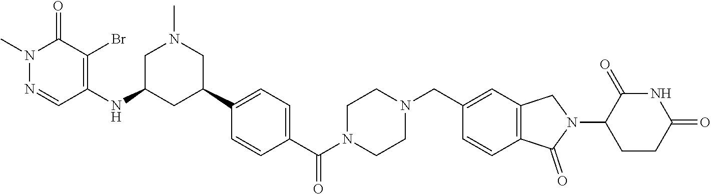 [SMILES: CN1C[C@H](Nc2cnn(C)c(=O)c2Br)C[C@H](c2ccc(C(=O)N3CCN(Cc4ccc5c(c4)CN(C4CCC(=O)NC4=O)C5=O)CC3)cc2)C1] |
| 71a | 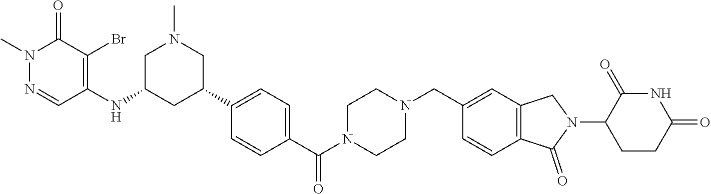 [SMILES: CN1C[C@@H](Nc2cnn(C)c(=O)c2Br)C[C@@H](c2ccc(C(=O)N3CCN(Cc4ccc5c(c4)CN(C4CCC(=O)NC4=O)C5=O)CC3)cc2)C1] |
| 71b | 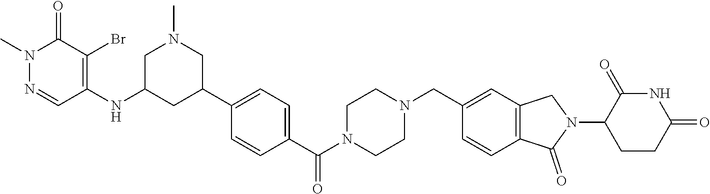 [SMILES: CN1CC(Nc2cnn(C)c(=O)c2Br)CC(c2ccc(C(=O)N3CCN(Cc4ccc5c(c4)CN(C4CCC(=O)NC4=O)C5=O)CC3)cc2)C1] |
| 72 | 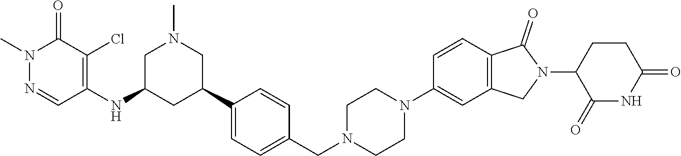 [SMILES: CN1C[C@H](Nc2cnn(C)c(=O)c2Cl)C[C@H](c2ccc(CN3CCN(c4ccc5c(c4)CN(C4CCC(=O)NC4=O)C5=O)CC3)cc2)C1] |
| 72a | 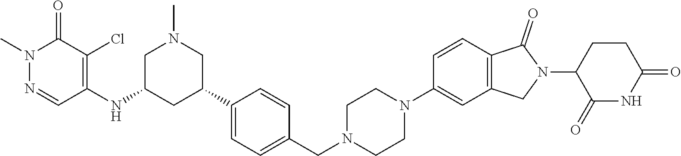 [SMILES: CN1C[C@@H](Nc2cnn(C)c(=O)c2Cl)C[C@@H](c2ccc(CN3CCN(c4ccc5c(c4)CN(C4CCC(=O)NC4=O)C5=O)CC3)cc2)C1] |
| 72b | 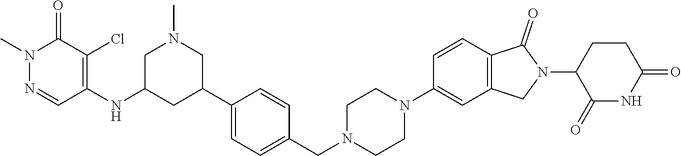 [SMILES: CN1CC(Nc2cnn(C)c(=O)c2Cl)CC(c2ccc(CN3CCN(c4ccc5c(c4)CN(C4CCC(=O)NC4=O)C5=O)CC3)cc2)C1] |
| 73 | 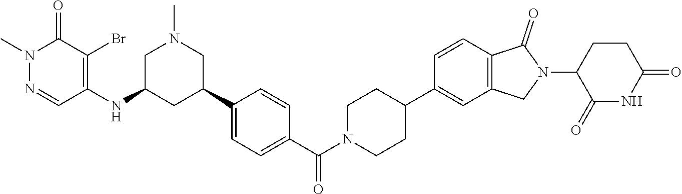 [SMILES: CN1C[C@H](Nc2cnn(C)c(=O)c2Br)C[C@H](c2ccc(C(=O)N3CCC(c4ccc5c(c4)CN(C4CCC(=O)NC4=O)C5=O)CC3)cc2)C1] |
| 73a | 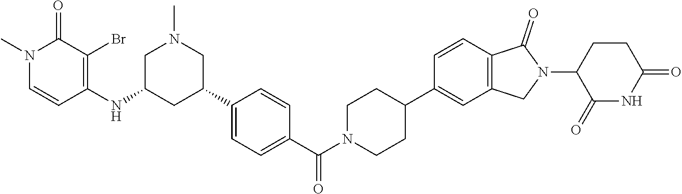 [SMILES: CN1C[C@@H](Nc2ccn(C)c(=O)c2Br)C[C@@H](c2ccc(C(=O)N3CCC(c4ccc5c(c4)CN(C4CCC(=O)NC4=O)C5=O)CC3)cc2)C1] |
| 73b | 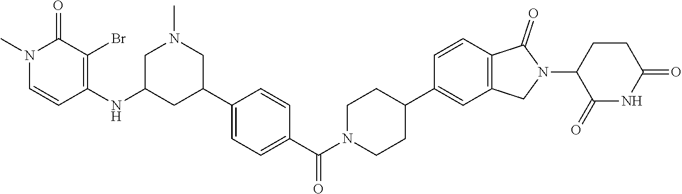 [SMILES: CN1CC(Nc2ccn(C)c(=O)c2Br)CC(c2ccc(C(=O)N3CCC(c4ccc5c(c4)CN(C4CCC(=O)NC4=O)C5=O)CC3)cc2)C1] |
| 74 | 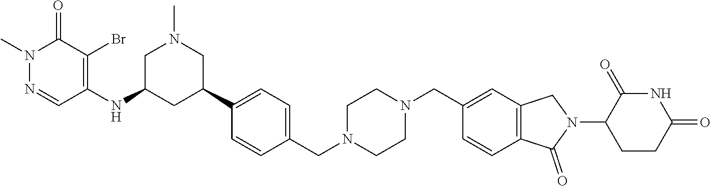 [SMILES: CN1C[C@H](Nc2cnn(C)c(=O)c2Br)C[C@H](c2ccc(CN3CCN(Cc4ccc5c(c4)CN(C4CCC(=O)NC4=O)C5=O)CC3)cc2)C1] |
| 74a | 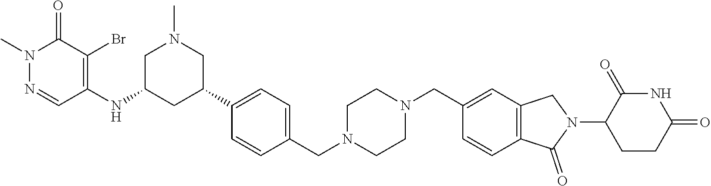 [SMILES: CN1C[C@@H](Nc2cnn(C)c(=O)c2Br)C[C@@H](c2ccc(CN3CCN(Cc4ccc5c(c4)CN(C4CCC(=O)NC4=O)C5=O)CC3)cc2)C1] |
| 74b | 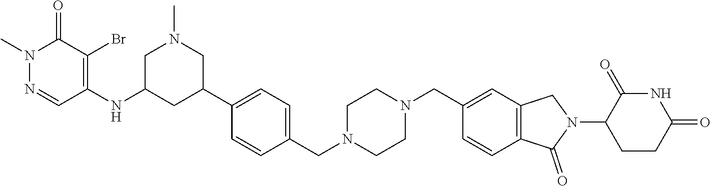 [SMILES: CN1CC(Nc2cnn(C)c(=O)c2Br)CC(c2ccc(CN3CCN(Cc4ccc5c(c4)CN(C4CCC(=O)NC4=O)C5=O)CC3)cc2)C1] |
| 75 | 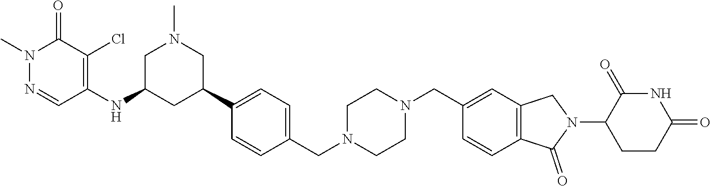 [SMILES: CN1C[C@H](Nc2cnn(C)c(=O)c2Cl)C[C@H](c2ccc(CN3CCN(Cc4ccc5c(c4)CN(C4CCC(=O)NC4=O)C5=O)CC3)cc2)C1] |
| 75a | 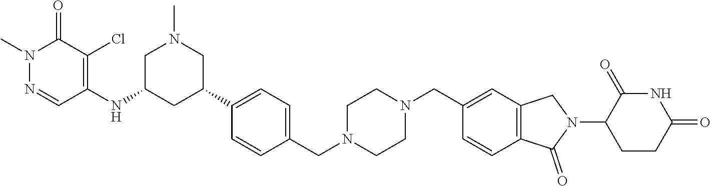 [SMILES: CN1C[C@@H](Nc2cnn(C)c(=O)c2Cl)C[C@@H](c2ccc(CN3CCN(Cc4ccc5c(c4)CN(C4CCC(=O)NC4=O)C5=O)CC3)cc2)C1] |
| 75b | 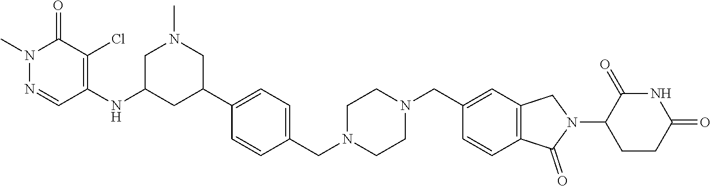 [SMILES: CN1CC(Nc2cnn(C)c(=O)c2Cl)CC(c2ccc(CN3CCN(Cc4ccc5c(c4)CN(C4CCC(=O)NC4=O)C5=O)CC3)cc2)C1] |
| 76 | 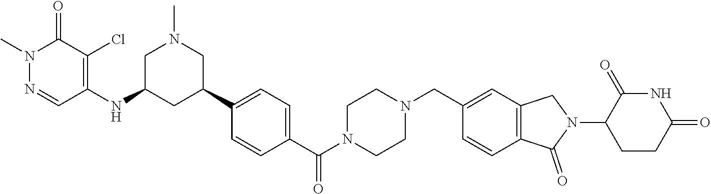 [SMILES: CN1C[C@H](Nc2cnn(C)c(=O)c2Cl)C[C@H](c2ccc(C(=O)N3CCN(Cc4ccc5c(c4)CN(C4CCC(=O)NC4=O)C5=O)CC3)cc2)C1] |
| 76a | 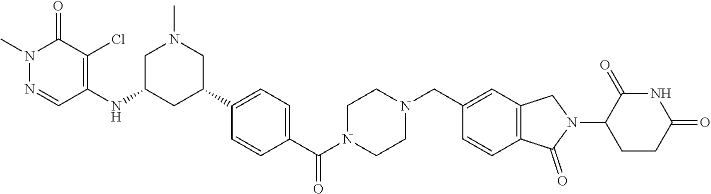 [SMILES: CN1C[C@@H](Nc2cnn(C)c(=O)c2Cl)C[C@@H](c2ccc(C(=O)N3CCN(Cc4ccc5c(c4)CN(C4CCC(=O)NC4=O)C5=O)CC3)cc2)C1] |
| 76b | 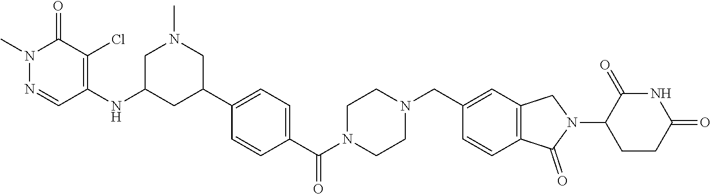 [SMILES: CN1CC(Nc2cnn(C)c(=O)c2Cl)CC(c2ccc(C(=O)N3CCN(Cc4ccc5c(c4)CN(C4CCC(=O)NC4=O)C5=O)CC3)cc2)C1] |
| 77 | 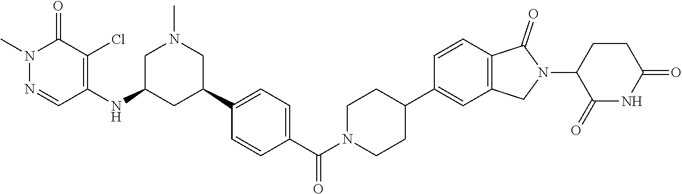 [SMILES: CN1C[C@H](Nc2cnn(C)c(=O)c2Cl)C[C@H](c2ccc(C(=O)N3CCC(c4ccc5c(c4)CN(C4CCC(=O)NC4=O)C5=O)CC3)cc2)C1] |
| 77a | 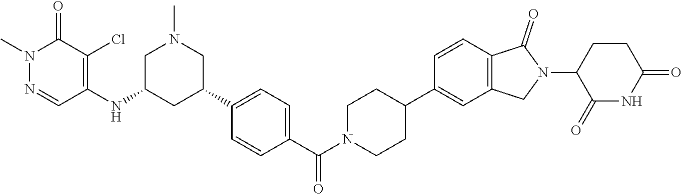 [SMILES: CN1C[C@@H](Nc2cnn(C)c(=O)c2Cl)C[C@@H](c2ccc(C(=O)N3CCC(c4ccc5c(c4)CN(C4CCC(=O)NC4=O)C5=O)CC3)cc2)C1] |
| 77b | 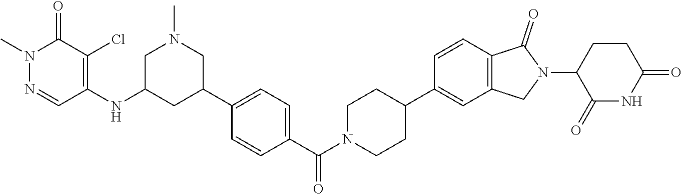 [SMILES: CN1CC(Nc2cnn(C)c(=O)c2Cl)CC(c2ccc(C(=O)N3CCC(c4ccc5c(c4)CN(C4CCC(=O)NC4=O)C5=O)CC3)cc2)C1] |
| 78 | 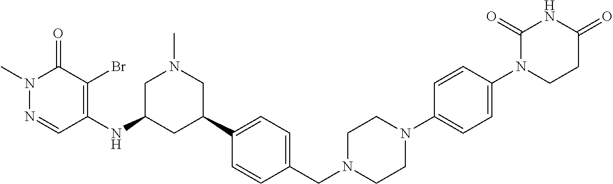 [SMILES: CN1C[C@H](Nc2cnn(C)c(=O)c2Br)C[C@H](c2ccc(CN3CCN(c4ccc(N5CCC(=O)NC5=O)cc4)CC3)cc2)C1] |
| 78a | 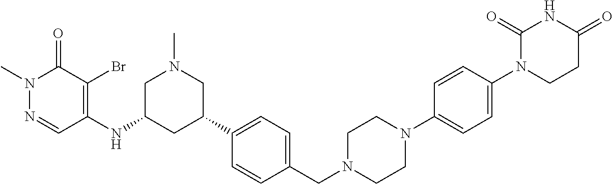 [SMILES: CN1C[C@@H](Nc2cnn(C)c(=O)c2Br)C[C@@H](c2ccc(CN3CCN(c4ccc(N5CCC(=O)NC5=O)cc4)CC3)cc2)C1] |
| 78b | 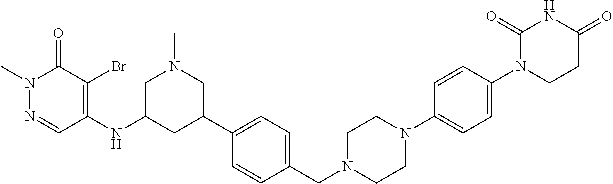 [SMILES: CN1CC(Nc2cnn(C)c(=O)c2Br)CC(c2ccc(CN3CCN(c4ccc(N5CCC(=O)NC5=O)cc4)CC3)cc2)C1] |
| 79 | 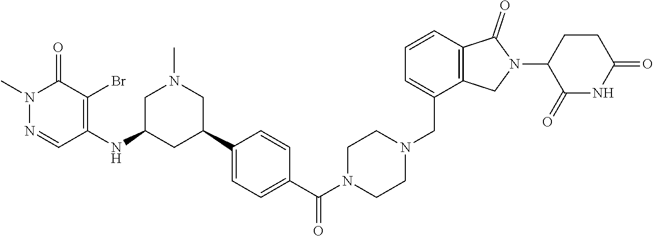 [SMILES: CN1C[C@H](Nc2cnn(C)c(=O)c2Br)C[C@H](c2ccc(C(=O)N3CCN(Cc4cccc5c4CN(C4CCC(=O)NC4=O)C5=O)CC3)cc2)C1] |
| 79a | 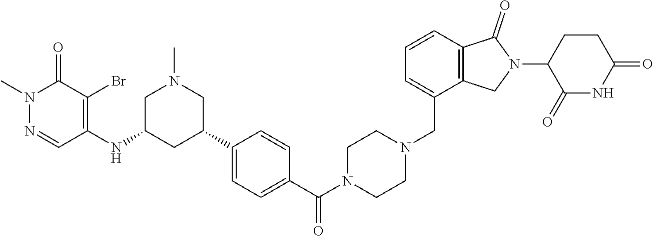 [SMILES: CN1C[C@@H](Nc2cnn(C)c(=O)c2Br)C[C@@H](c2ccc(C(=O)N3CCN(Cc4cccc5c4CN(C4CCC(=O)NC4=O)C5=O)CC3)cc2)C1] |
| 79b | 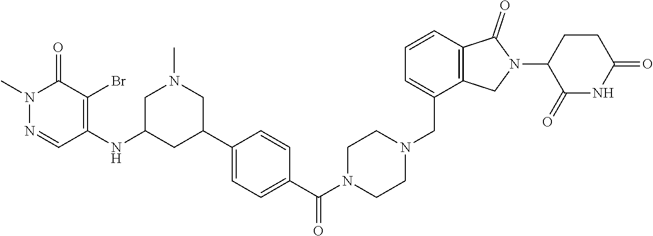 [SMILES: CN1CC(Nc2cnn(C)c(=O)c2Br)CC(c2ccc(C(=O)N3CCN(Cc4cccc5c4CN(C4CCC(=O)NC4=O)C5=O)CC3)cc2)C1] |
| 80 | 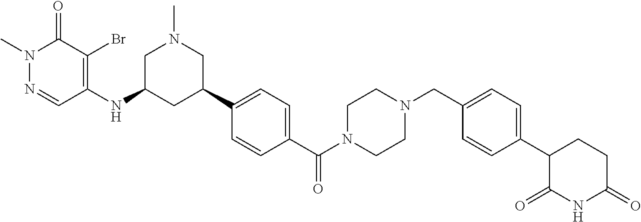 [SMILES: CN1C[C@H](Nc2cnn(C)c(=O)c2Br)C[C@H](c2ccc(C(=O)N3CCN(Cc4ccc(C5CCC(=O)NC5=O)cc4)CC3)cc2)C1] |
| 80a | 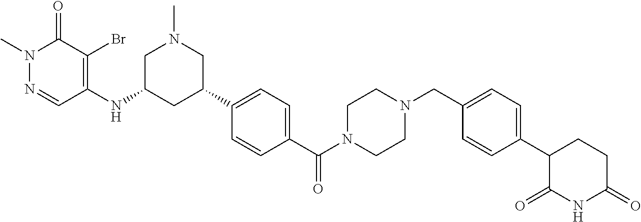 [SMILES: CN1C[C@@H](Nc2cnn(C)c(=O)c2Br)C[C@@H](c2ccc(C(=O)N3CCN(Cc4ccc(C5CCC(=O)NC5=O)cc4)CC3)cc2)C1] |
| 80b | 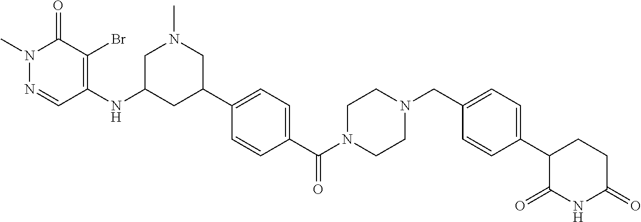 [SMILES: CN1CC(Nc2cnn(C)c(=O)c2Br)CC(c2ccc(C(=O)N3CCN(Cc4ccc(C5CCC(=O)NC5=O)cc4)CC3)cc2)C1] |
| 81 | 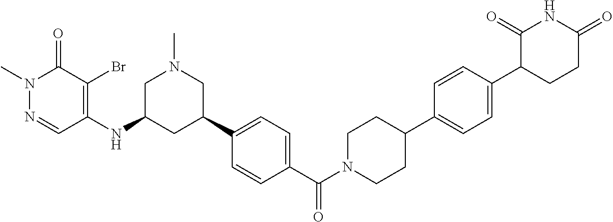 [SMILES: CN1C[C@H](Nc2cnn(C)c(=O)c2Br)C[C@H](c2ccc(C(=O)N3CCC(c4ccc(C5CCC(=O)NC5=O)cc4)CC3)cc2)C1] |
| 81a | 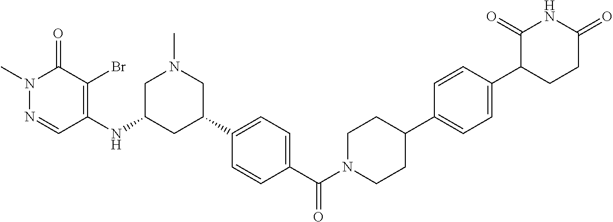 [SMILES: CN1C[C@@H](Nc2cnn(C)c(=O)c2Br)C[C@@H](c2ccc(C(=O)N3CCC(c4ccc(C5CCC(=O)NC5=O)cc4)CC3)cc2)C1] |
| 81b | 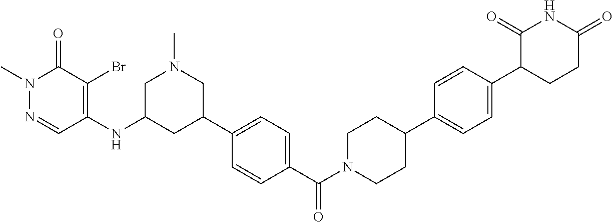 [SMILES: CN1CC(Nc2cnn(C)c(=O)c2Br)CC(c2ccc(C(=O)N3CCC(c4ccc(C5CCC(=O)NC5=O)cc4)CC3)cc2)C1] |
| 82 | 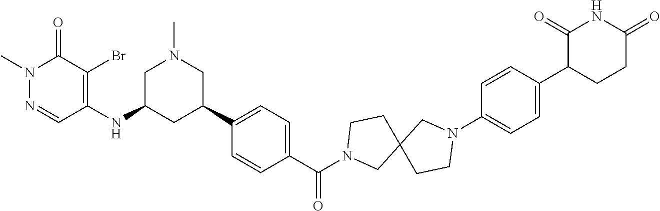 [SMILES: CN1C[C@H](Nc2cnn(C)c(=O)c2Br)C[C@H](c2ccc(C(=O)N3CCC4(CCN(c5ccc(C6CCC(=O)NC6=O)cc5)C4)C3)cc2)C1] |
| 82a | 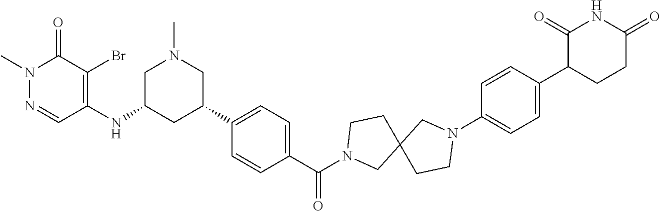 [SMILES: CN1C[C@@H](Nc2cnn(C)c(=O)c2Br)C[C@@H](c2ccc(C(=O)N3CCC4(CCN(c5ccc(C6CCC(=O)NC6=O)cc5)C4)C3)cc2)C1] |
| 82b | 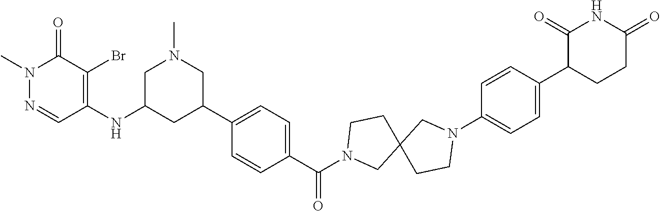 [SMILES: CN1CC(Nc2cnn(C)c(=O)c2Br)CC(c2ccc(C(=O)N3CCC4(CCN(c5ccc(C6CCC(=O)NC6=O)cc5)C4)C3)cc2)C1] |
| 83 | 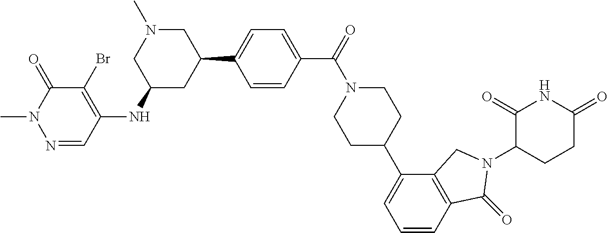 [SMILES: CN1C[C@H](Nc2cnn(C)c(=O)c2Br)C[C@H](c2ccc(C(=O)N3CCC(c4cccc5c4CN(C4CCC(=O)NC4=O)C5=O)CC3)cc2)C1] |
| 83a | 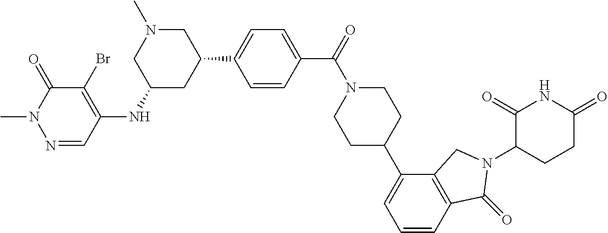 [SMILES: CN1C[C@@H](Nc2cnn(C)c(=O)c2Br)C[C@@H](c2ccc(C(=O)N3CCC(c4cccc5c4CN(C4CCC(=O)NC4=O)C5=O)CC3)cc2)C1] |
| 83b | 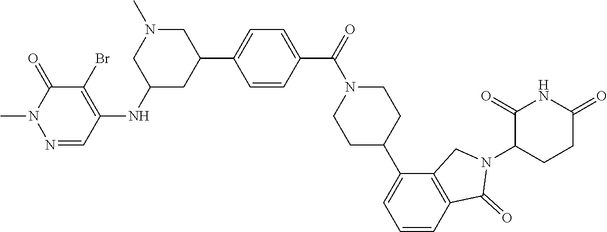 [SMILES: CN1CC(Nc2cnn(C)c(=O)c2Br)CC(c2ccc(C(=O)N3CCC(c4cccc5c4CN(C4CCC(=O)NC4=O)C5=O)CC3)cc2)C1] |
| 84 | 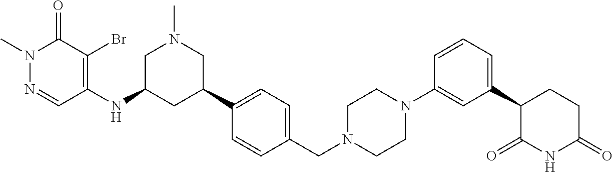 [SMILES: CN1C[C@H](Nc2cnn(C)c(=O)c2Br)C[C@H](c2ccc(CN3CCN(c4cccc([C@H]5CCC(=O)NC5=O)c4)CC3)cc2)C1] |
| 84-1 | 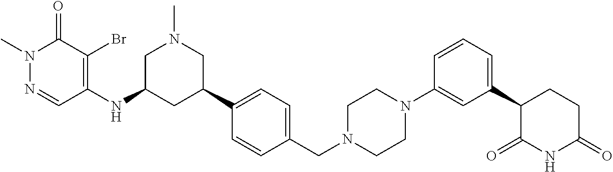 [SMILES: CN1C[C@H](Nc2cnn(C)c(=O)c2Br)C[C@H](c2ccc(CN3CCN(c4cccc([C@H]5CCC(=O)NC5=O)c4)CC3)cc2)C1] |
| 84-2 | 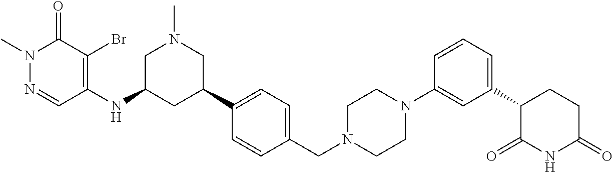 [SMILES: CN1C[C@H](Nc2cnn(C)c(=O)c2Br)C[C@H](c2ccc(CN3CCN(c4cccc([C@@H]5CCC(=O)NC5=O)c4)CC3)cc2)C1] |
| 84a | 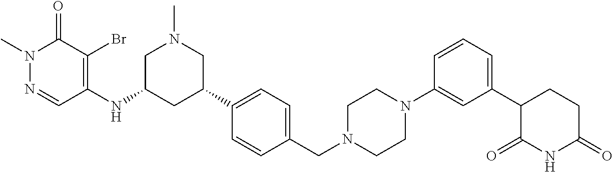 [SMILES: CN1C[C@@H](Nc2cnn(C)c(=O)c2Br)C[C@@H](c2ccc(CN3CCN(c4cccc(C5CCC(=O)NC5=O)c4)CC3)cc2)C1] |
| 84b | 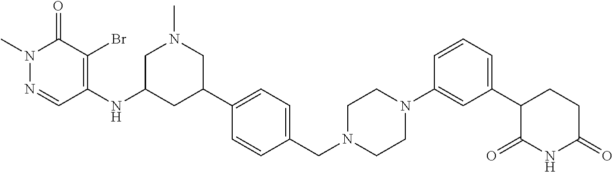 [SMILES: CN1CC(Nc2cnn(C)c(=O)c2Br)CC(c2ccc(CN3CCN(c4cccc(C5CCC(=O)NC5=O)c4)CC3)cc2)C1] |
| 85 | 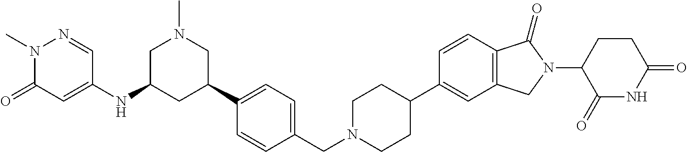 [SMILES: CN1C[C@H](Nc2cnn(C)c(=O)c2)C[C@H](c2ccc(CN3CCC(c4ccc5c(c4)CN(C4CCC(=O)NC4=O)C5=O)CC3)cc2)C1] |
| 85a | 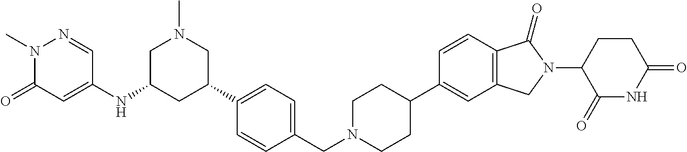 [SMILES: CN1C[C@@H](Nc2cnn(C)c(=O)c2)C[C@@H](c2ccc(CN3CCC(c4ccc5c(c4)CN(C4CCC(=O)NC4=O)C5=O)CC3)cc2)C1] |
| 85b | 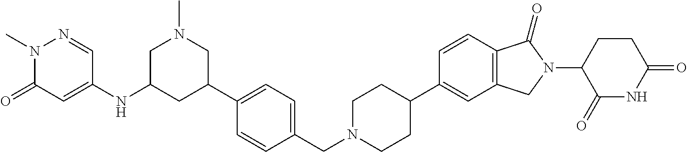 [SMILES: CN1CC(Nc2cnn(C)c(=O)c2)CC(c2ccc(CN3CCC(c4ccc5c(c4)CN(C4CCC(=O)NC4=O)C5=O)CC3)cc2)C1] |
| 86 | 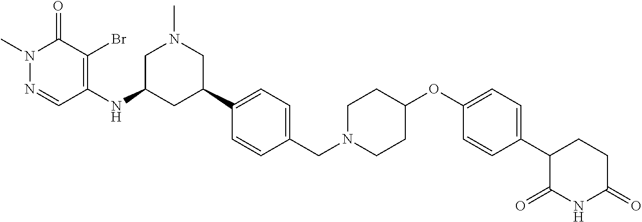 [SMILES: CN1C[C@H](Nc2cnn(C)c(=O)c2Br)C[C@H](c2ccc(CN3CCC(Oc4ccc(C5CCC(=O)NC5=O)cc4)CC3)cc2)C1] |
| 86a | 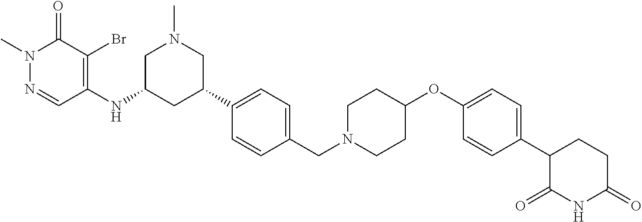 [SMILES: CN1C[C@@H](Nc2cnn(C)c(=O)c2Br)C[C@@H](c2ccc(CN3CCC(Oc4ccc(C5CCC(=O)NC5=O)cc4)CC3)cc2)C1] |
| 86b | 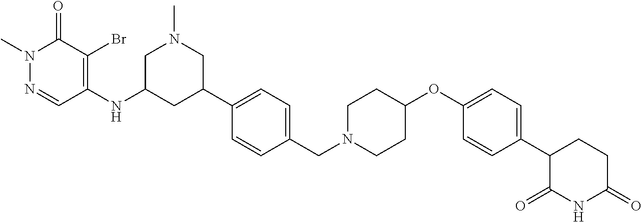 [SMILES: CN1CC(Nc2cnn(C)c(=O)c2Br)CC(c2ccc(CN3CCC(Oc4ccc(C5CCC(=O)NC5=O)cc4)CC3)cc2)C1] |
| 87 | 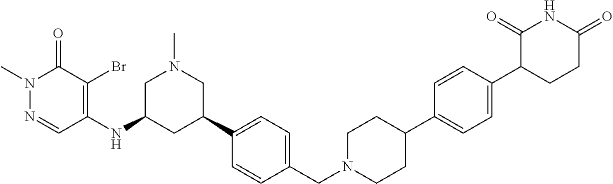 [SMILES: CN1C[C@H](Nc2cnn(C)c(=O)c2Br)C[C@H](c2ccc(CN3CCC(c4ccc(C5CCC(=O)NC5=O)cc4)CC3)cc2)C1] |
| 87a | 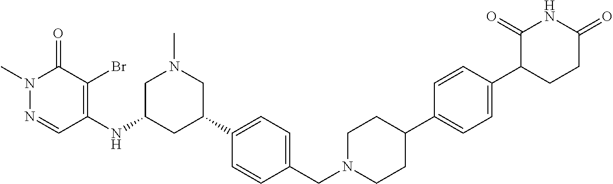 [SMILES: CN1C[C@@H](Nc2cnn(C)c(=O)c2Br)C[C@@H](c2ccc(CN3CCC(c4ccc(C5CCC(=O)NC5=O)cc4)CC3)cc2)C1] |
| 87b | 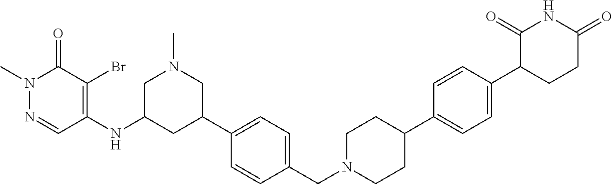 [SMILES: CN1CC(Nc2cnn(C)c(=O)c2Br)CC(c2ccc(CN3CCC(c4ccc(C5CCC(=O)NC5=O)cc4)CC3)cc2)C1] |
| 88 | 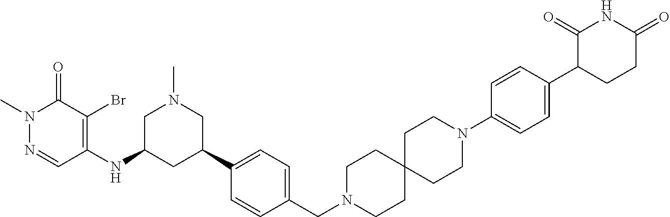 [SMILES: CN1C[C@H](Nc2cnn(C)c(=O)c2Br)C[C@H](c2ccc(CN3CCC4(CC3)CCN(c3ccc(C5CCC(=O)NC5=O)cc3)CC4)cc2)C1] |
| 88a | 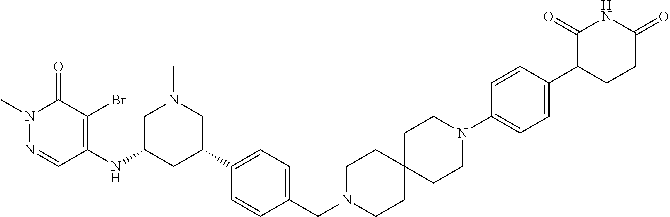 [SMILES: CN1C[C@@H](Nc2cnn(C)c(=O)c2Br)C[C@@H](c2ccc(CN3CCC4(CC3)CCN(c3ccc(C5CCC(=O)NC5=O)cc3)CC4)cc2)C1] |
| 88b | 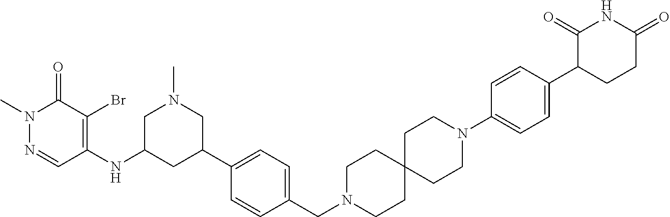 [SMILES: CN1CC(Nc2cnn(C)c(=O)c2Br)CC(c2ccc(CN3CCC4(CC3)CCN(c3ccc(C5CCC(=O)NC5=O)cc3)CC4)cc2)C1] |
| 89 | 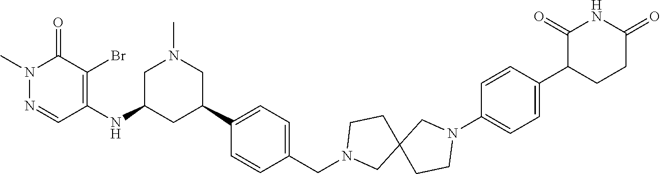 [SMILES: CN1C[C@H](Nc2cnn(C)c(=O)c2Br)C[C@H](c2ccc(CN3CCC4(CCN(c5ccc(C6CCC(=O)NC6=O)cc5)C4)C3)cc2)C1] |
| 89a | 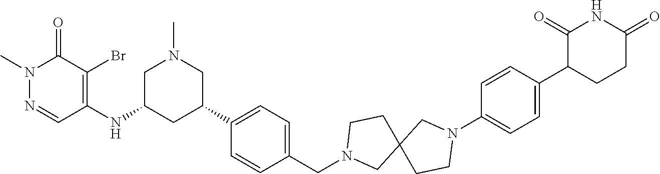 [SMILES: CN1C[C@@H](Nc2cnn(C)c(=O)c2Br)C[C@@H](c2ccc(CN3CCC4(CCN(c5ccc(C6CCC(=O)NC6=O)cc5)C4)C3)cc2)C1] |
| 89b | 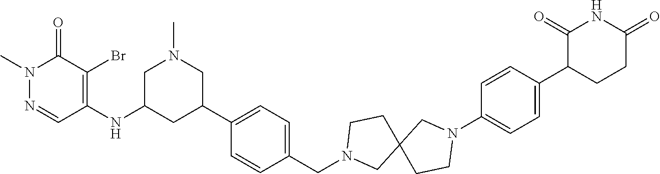 [SMILES: CN1CC(Nc2cnn(C)c(=O)c2Br)CC(c2ccc(CN3CCC4(CCN(c5ccc(C6CCC(=O)NC6=O)cc5)C4)C3)cc2)C1] |
| 90 | 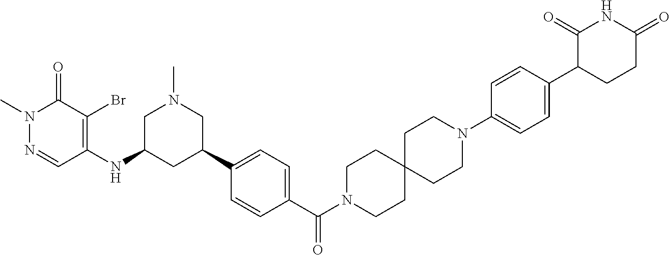 [SMILES: CN1C[C@H](Nc2cnn(C)c(=O)c2Br)C[C@H](c2ccc(C(=O)N3CCC4(CC3)CCN(c3ccc(C5CCC(=O)NC5=O)cc3)CC4)cc2)C1] |
| 90-1 | 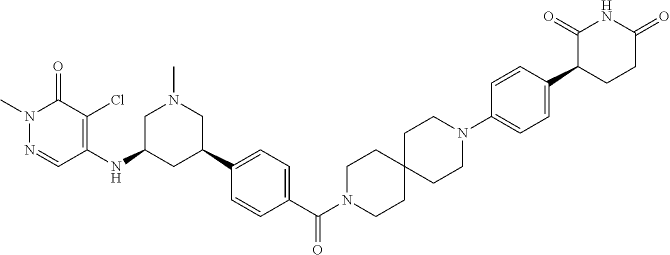 [SMILES: CN1C[C@H](Nc2cnn(C)c(=O)c2Cl)C[C@H](c2ccc(C(=O)N3CCC4(CC3)CCN(c3ccc([C@@H]5CCC(=O)NC5=O)cc3)CC4)cc2)C1] |
| 90-2 | 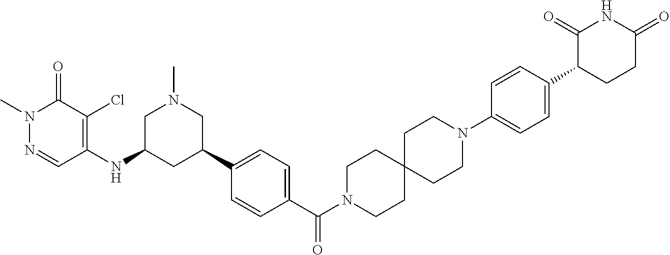 [SMILES: CN1C[C@H](Nc2cnn(C)c(=O)c2Cl)C[C@H](c2ccc(C(=O)N3CCC4(CC3)CCN(c3ccc([C@H]5CCC(=O)NC5=O)cc3)CC4)cc2)C1] |
| 90a | 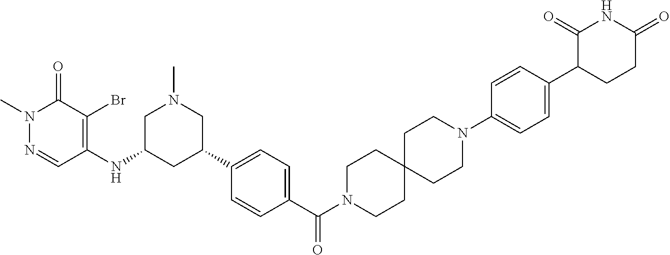 [SMILES: CN1C[C@@H](Nc2cnn(C)c(=O)c2Br)C[C@@H](c2ccc(C(=O)N3CCC4(CC3)CCN(c3ccc(C5CCC(=O)NC5=O)cc3)CC4)cc2)C1] |
| 90b | 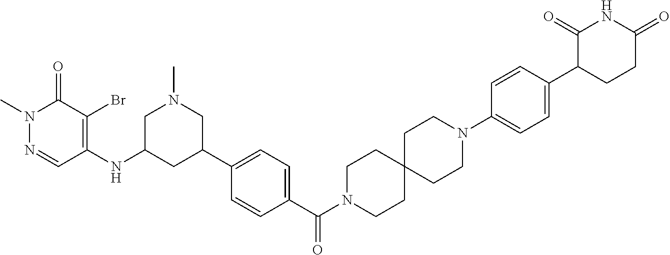 [SMILES: CN1CC(Nc2cnn(C)c(=O)c2Br)CC(c2ccc(C(=O)N3CCC4(CC3)CCN(c3ccc(C5CCC(=O)NC5=O)cc3)CC4)cc2)C1] |
| 91 | 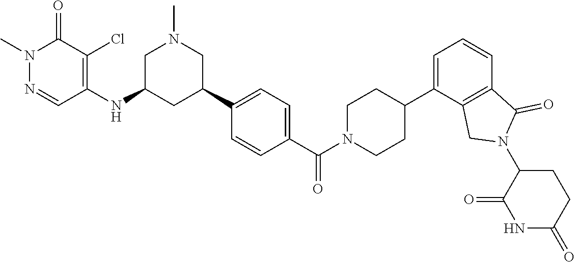 [SMILES: CN1C[C@H](Nc2cnn(C)c(=O)c2Cl)C[C@H](c2ccc(C(=O)N3CCC(c4cccc5c4CN(C4CCC(=O)NC4=O)C5=O)CC3)cc2)C1] |
| 91a | 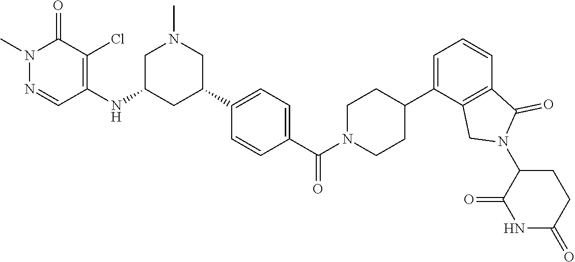 [SMILES: CN1C[C@@H](Nc2cnn(C)c(=O)c2Cl)C[C@@H](c2ccc(C(=O)N3CCC(c4cccc5c4CN(C4CCC(=O)NC4=O)C5=O)CC3)cc2)C1] |
| 91b | 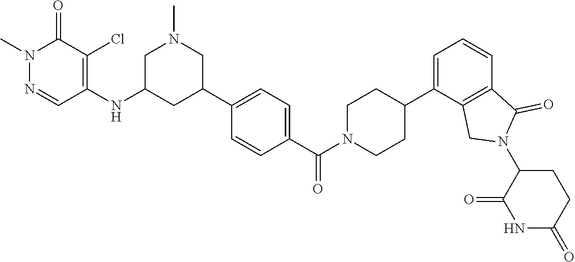 [SMILES: CN1CC(Nc2cnn(C)c(=O)c2Cl)CC(c2ccc(C(=O)N3CCC(c4cccc5c4CN(C4CCC(=O)NC4=O)C5=O)CC3)cc2)C1] |
| 92 | 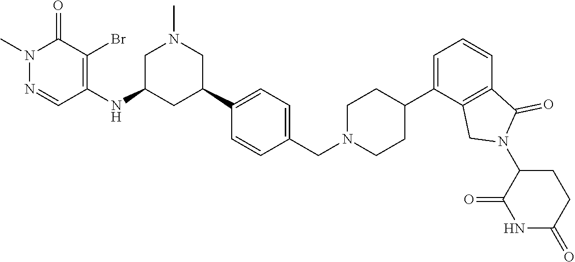 [SMILES: CN1C[C@H](Nc2cnn(C)c(=O)c2Br)C[C@H](c2ccc(CN3CCC(c4cccc5c4CN(C4CCC(=O)NC4=O)C5=O)CC3)cc2)C1] |
| 92a | 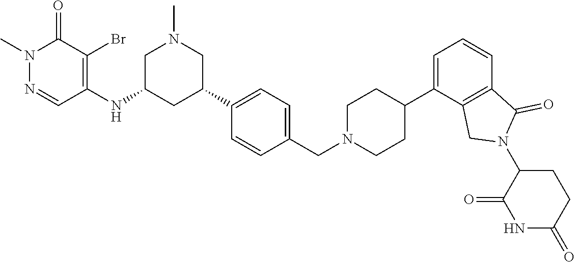 [SMILES: CN1C[C@@H](Nc2cnn(C)c(=O)c2Br)C[C@@H](c2ccc(CN3CCC(c4cccc5c4CN(C4CCC(=O)NC4=O)C5=O)CC3)cc2)C1] |
| 92b | 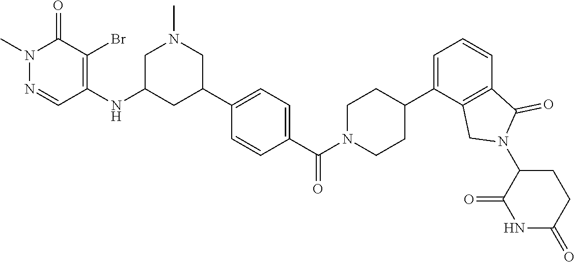 [SMILES: CN1CC(Nc2cnn(C)c(=O)c2Br)CC(c2ccc(C(=O)N3CCC(c4cccc5c4CN(C4CCC(=O)NC4=O)C5=O)CC3)cc2)C1] |
| 93 | 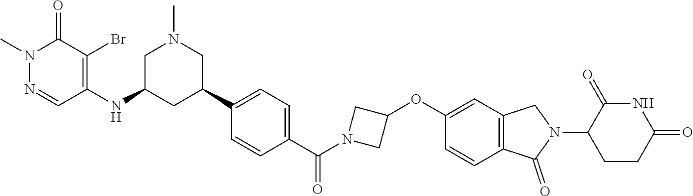 [SMILES: CN1C[C@H](Nc2cnn(C)c(=O)c2Br)C[C@H](c2ccc(C(=O)N3CC(Oc4ccc5c(c4)CN(C4CCC(=O)NC4=O)C5=O)C3)cc2)C1] |
| 93a | 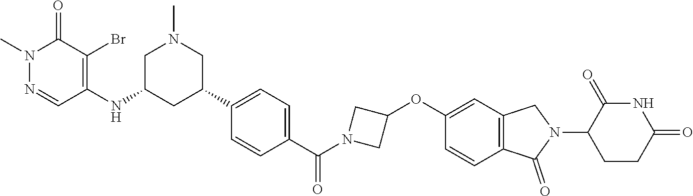 [SMILES: CN1C[C@@H](Nc2cnn(C)c(=O)c2Br)C[C@@H](c2ccc(C(=O)N3CC(Oc4ccc5c(c4)CN(C4CCC(=O)NC4=O)C5=O)C3)cc2)C1] |
| 93b | 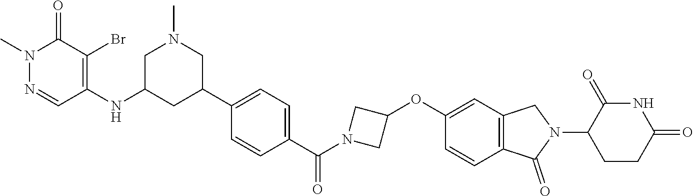 [SMILES: CN1CC(Nc2cnn(C)c(=O)c2Br)CC(c2ccc(C(=O)N3CC(Oc4ccc5c(c4)CN(C4CCC(=O)NC4=O)C5=O)C3)cc2)C1] |
| 94 | 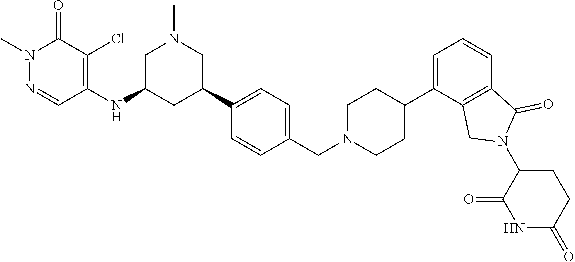 [SMILES: CN1C[C@H](Nc2cnn(C)c(=O)c2Cl)C[C@H](c2ccc(CN3CCC(c4cccc5c4CN(C4CCC(=O)NC4=O)C5=O)CC3)cc2)C1] |
| 94a | 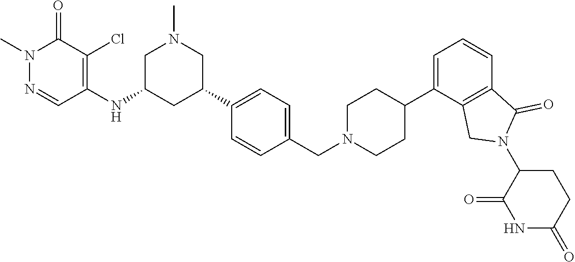 [SMILES: CN1C[C@@H](Nc2cnn(C)c(=O)c2Cl)C[C@@H](c2ccc(CN3CCC(c4cccc5c4CN(C4CCC(=O)NC4=O)C5=O)CC3)cc2)C1] |
| 94b | 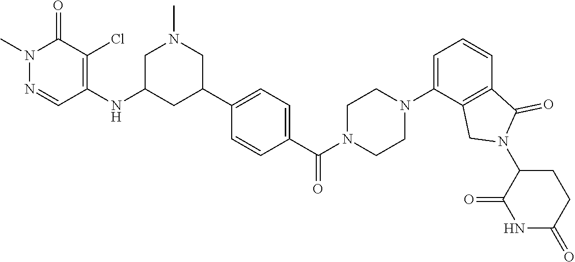 [SMILES: CN1CC(Nc2cnn(C)c(=O)c2Cl)CC(c2ccc(C(=O)N3CCN(c4cccc5c4CN(C4CCC(=O)NC4=O)C5=O)CC3)cc2)C1] |
| 95 | 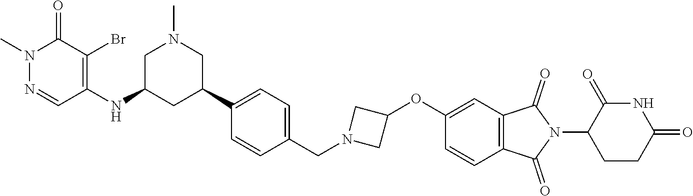 [SMILES: CN1C[C@H](Nc2cnn(C)c(=O)c2Br)C[C@H](c2ccc(CN3CC(Oc4ccc5c(c4)C(=O)N(C4CCC(=O)NC4=O)C5=O)C3)cc2)C1] |
| 95a | 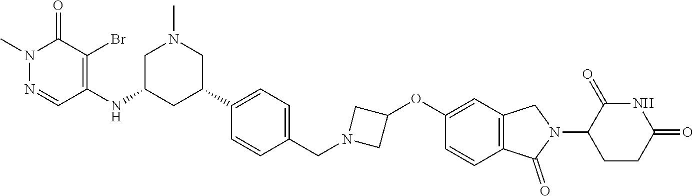 [SMILES: CN1C[C@@H](Nc2cnn(C)c(=O)c2Br)C[C@@H](c2ccc(CN3CC(Oc4ccc5c(c4)CN(C4CCC(=O)NC4=O)C5=O)C3)cc2)C1] |
| 95b | 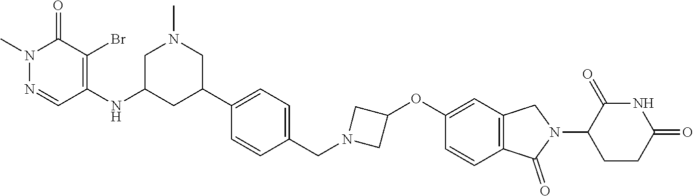 [SMILES: CN1CC(Nc2cnn(C)c(=O)c2Br)CC(c2ccc(CN3CC(Oc4ccc5c(c4)CN(C4CCC(=O)NC4=O)C5=O)C3)cc2)C1] |
| 96 | 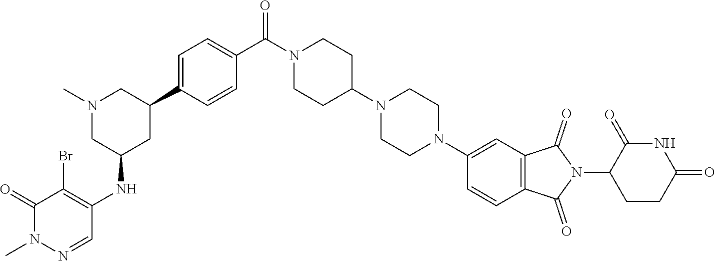 [SMILES: CN1C[C@H](Nc2cnn(C)c(=O)c2Br)C[C@H](c2ccc(C(=O)N3CCC(N4CCN(c5ccc6c(c5)C(=O)N(C5CCC(=O)NC5=O)C6=O)CC4)CC3)cc2)C1] |
| 96a | 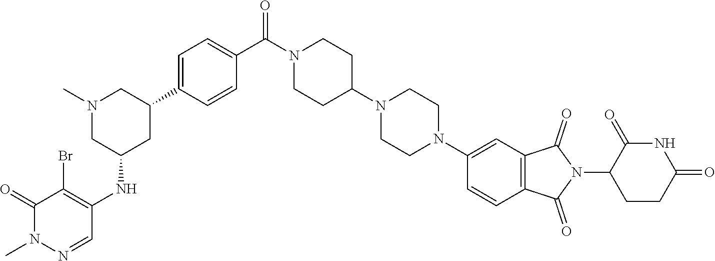 [SMILES: CN1C[C@@H](Nc2cnn(C)c(=O)c2Br)C[C@@H](c2ccc(C(=O)N3CCC(N4CCN(c5ccc6c(c5)C(=O)N(C5CCC(=O)NC5=O)C6=O)CC4)CC3)cc2)C1] |
| 96b | 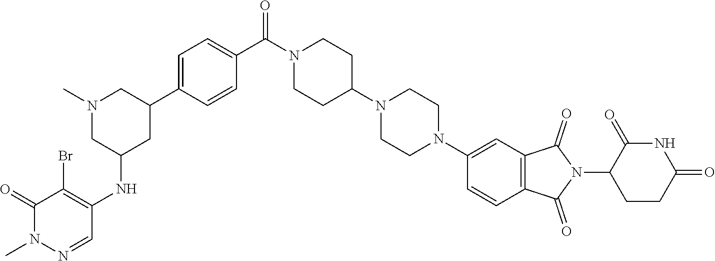 [SMILES: CN1CC(Nc2cnn(C)c(=O)c2Br)CC(c2ccc(C(=O)N3CCC(N4CCN(c5ccc6c(c5)C(=O)N(C5CCC(=O)NC5=O)C6=O)CC4)CC3)cc2)C1] |
| 97 | 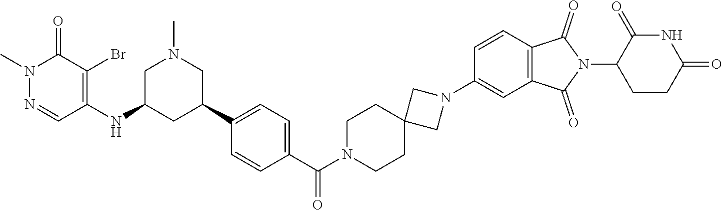 [SMILES: CN1C[C@H](Nc2cnn(C)c(=O)c2Br)C[C@H](c2ccc(C(=O)N3CCC4(CC3)CN(c3ccc5c(c3)C(=O)N(C3CCC(=O)NC3=O)C5=O)C4)cc2)C1] |
| 97a | 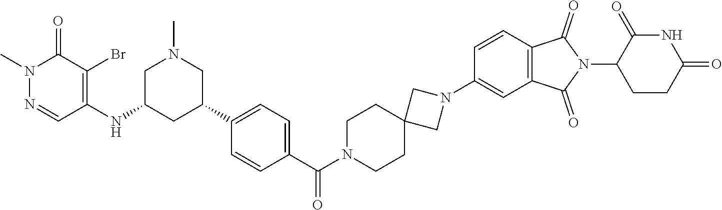 [SMILES: CN1C[C@@H](Nc2cnn(C)c(=O)c2Br)C[C@@H](c2ccc(C(=O)N3CCC4(CC3)CN(c3ccc5c(c3)C(=O)N(C3CCC(=O)NC3=O)C5=O)C4)cc2)C1] |
| 97b | 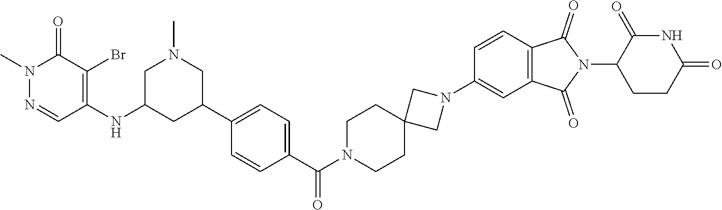 [SMILES: CN1CC(Nc2cnn(C)c(=O)c2Br)CC(c2ccc(C(=O)N3CCC4(CC3)CN(c3ccc5c(c3)C(=O)N(C3CCC(=O)NC3=O)C5=O)C4)cc2)C1] |
| 98 | 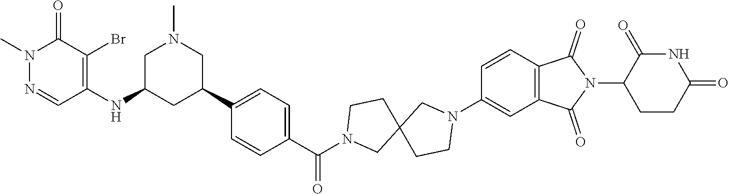 [SMILES: CN1C[C@H](Nc2cnn(C)c(=O)c2Br)C[C@H](c2ccc(C(=O)N3CCC4(CCN(c5ccc6c(c5)C(=O)N(C5CCC(=O)NC5=O)C6=O)C4)C3)cc2)C1] |
| 98a | 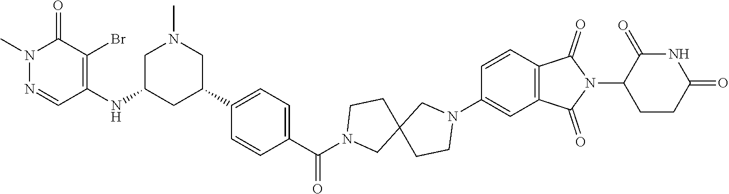 [SMILES: CN1C[C@@H](Nc2cnn(C)c(=O)c2Br)C[C@@H](c2ccc(C(=O)N3CCC4(CCN(c5ccc6c(c5)C(=O)N(C5CCC(=O)NC5=O)C6=O)C4)C3)cc2)C1] |
| 98b | 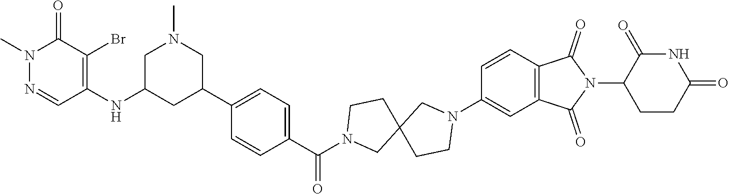 [SMILES: CN1CC(Nc2cnn(C)c(=O)c2Br)CC(c2ccc(C(=O)N3CCC4(CCN(c5ccc6c(c5)C(=O)N(C5CCC(=O)NC5=O)C6=O)C4)C3)cc2)C1] |
| 99 | 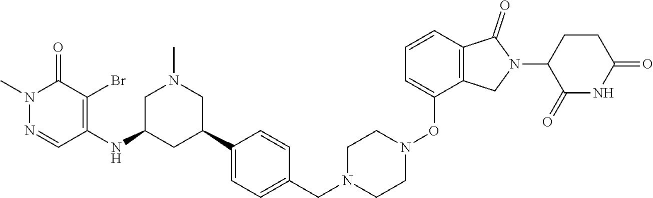 [SMILES: CN1C[C@H](Nc2cnn(C)c(=O)c2Br)C[C@H](c2ccc(CN3CCN(Oc4cccc5c4CN(C4CCC(=O)NC4=O)C5=O)CC3)cc2)C1] |
| 99a | 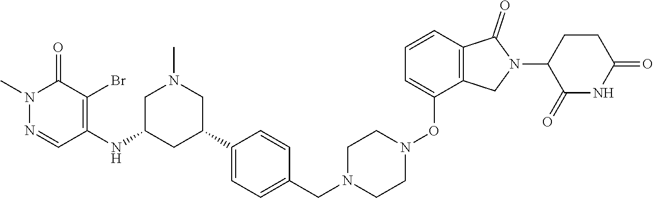 [SMILES: CN1C[C@@H](Nc2cnn(C)c(=O)c2Br)C[C@@H](c2ccc(CN3CCN(Oc4cccc5c4CN(C4CCC(=O)NC4=O)C5=O)CC3)cc2)C1] |
| 99b | 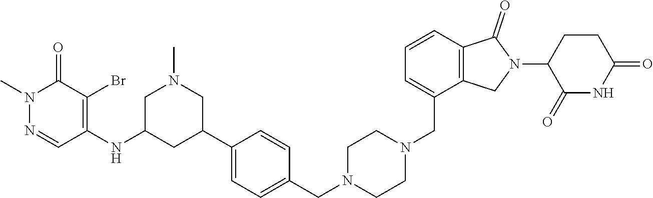 [SMILES: CN1CC(Nc2cnn(C)c(=O)c2Br)CC(c2ccc(CN3CCN(Cc4cccc5c4CN(C4CCC(=O)NC4=O)C5=O)CC3)cc2)C1] |
| 100 | 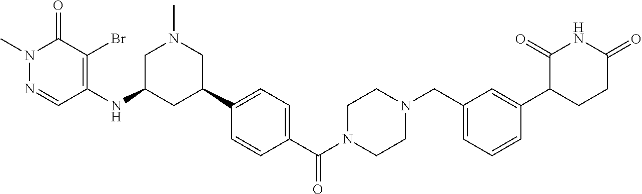 [SMILES: CN1C[C@H](Nc2cnn(C)c(=O)c2Br)C[C@H](c2ccc(C(=O)N3CCN(Cc4cccc(C5CCC(=O)NC5=O)c4)CC3)cc2)C1] |
| 100a | 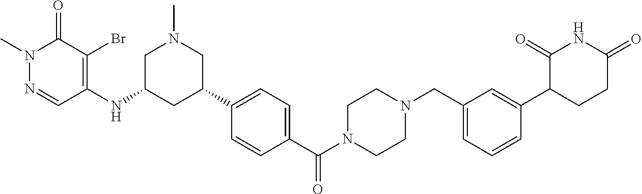 [SMILES: CN1C[C@@H](Nc2cnn(C)c(=O)c2Br)C[C@@H](c2ccc(C(=O)N3CCN(Cc4cccc(C5CCC(=O)NC5=O)c4)CC3)cc2)C1] |
| 100b | 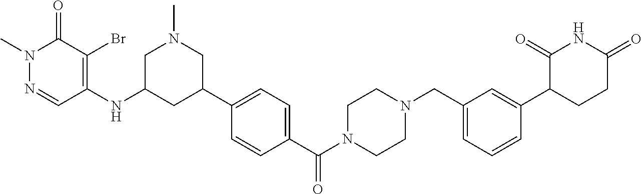 [SMILES: CN1CC(Nc2cnn(C)c(=O)c2Br)CC(c2ccc(C(=O)N3CCN(Cc4cccc(C5CCC(=O)NC5=O)c4)CC3)cc2)C1] |
| 101 | 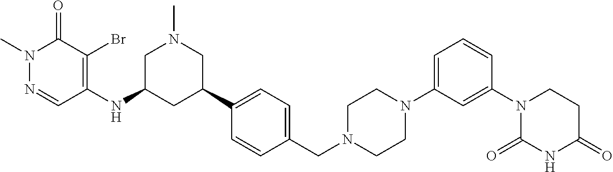 [SMILES: CN1C[C@H](Nc2cnn(C)c(=O)c2Br)C[C@H](c2ccc(CN3CCN(c4cccc(N5CCC(=O)NC5=O)c4)CC3)cc2)C1] |
| 101a | 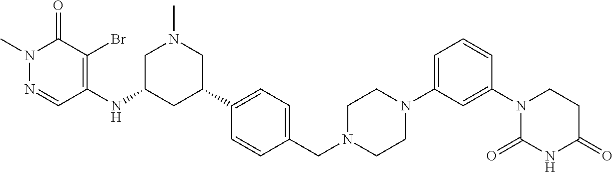 [SMILES: CN1C[C@@H](Nc2cnn(C)c(=O)c2Br)C[C@@H](c2ccc(CN3CCN(c4cccc(N5CCC(=O)NC5=O)c4)CC3)cc2)C1] |
| 101b | 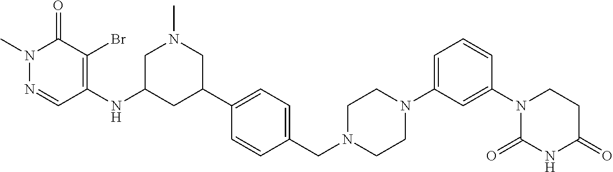 [SMILES: CN1CC(Nc2cnn(C)c(=O)c2Br)CC(c2ccc(CN3CCN(c4cccc(N5CCC(=O)NC5=O)c4)CC3)cc2)C1] |
| 102 | 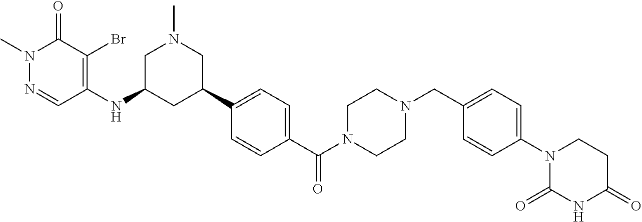 [SMILES: CN1C[C@H](Nc2cnn(C)c(=O)c2Br)C[C@H](c2ccc(C(=O)N3CCN(Cc4ccc(N5CCC(=O)NC5=O)cc4)CC3)cc2)C1] |
| 102a | 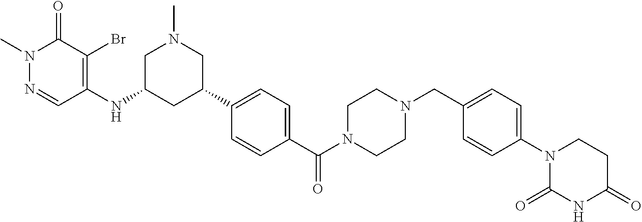 [SMILES: CN1C[C@@H](Nc2cnn(C)c(=O)c2Br)C[C@@H](c2ccc(C(=O)N3CCN(Cc4ccc(N5CCC(=O)NC5=O)cc4)CC3)cc2)C1] |
| 102b | 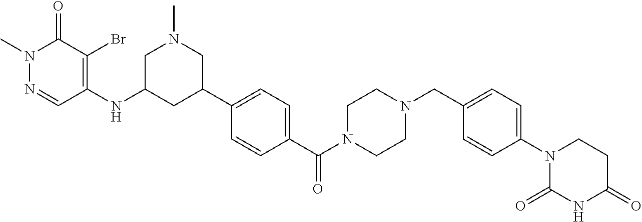 [SMILES: CN1CC(Nc2cnn(C)c(=O)c2Br)CC(c2ccc(C(=O)N3CCN(Cc4ccc(N5CCC(=O)NC5=O)cc4)CC3)cc2)C1] |
| 103 | 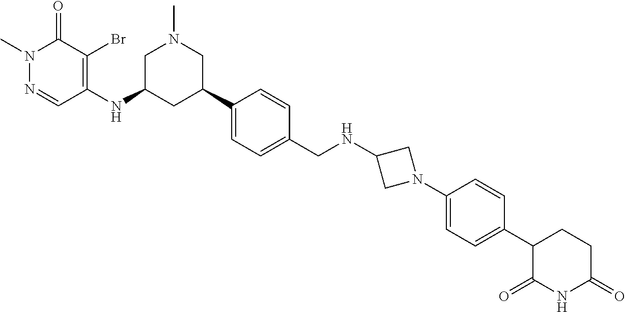 [SMILES: CN1C[C@H](Nc2cnn(C)c(=O)c2Br)C[C@H](c2ccc(CNC3CN(c4ccc(C5CCC(=O)NC5=O)cc4)C3)cc2)C1] |
| 103a | 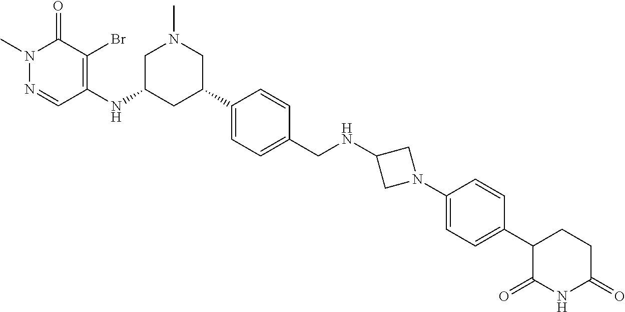 [SMILES: CN1C[C@@H](Nc2cnn(C)c(=O)c2Br)C[C@@H](c2ccc(CNC3CN(c4ccc(C5CCC(=O)NC5=O)cc4)C3)cc2)C1] |
| 103b | 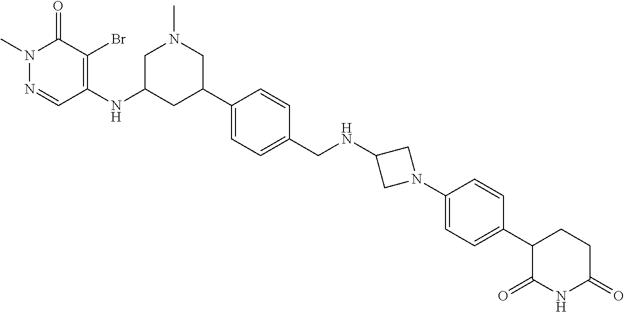 [SMILES: CN1CC(Nc2cnn(C)c(=O)c2Br)CC(c2ccc(CNC3CN(c4ccc(C5CCC(=O)NC5=O)cc4)C3)cc2)C1] |
| 104 | 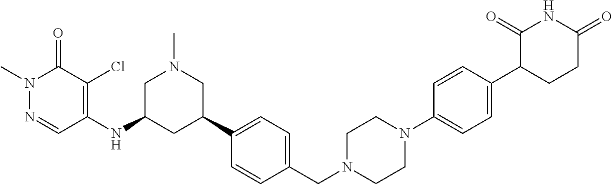 [SMILES: CN1C[C@H](Nc2cnn(C)c(=O)c2Cl)C[C@H](c2ccc(CN3CCN(c4ccc(C5CCC(=O)NC5=O)cc4)CC3)cc2)C1] |
| 104a | 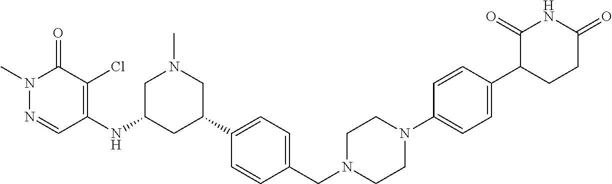 [SMILES: CN1C[C@@H](Nc2cnn(C)c(=O)c2Cl)C[C@@H](c2ccc(CN3CCN(c4ccc(C5CCC(=O)NC5=O)cc4)CC3)cc2)C1] |
| 104b | 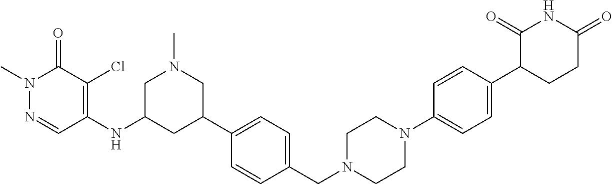 [SMILES: CN1CC(Nc2cnn(C)c(=O)c2Cl)CC(c2ccc(CN3CCN(c4ccc(C5CCC(=O)NC5=O)cc4)CC3)cc2)C1] |
| 105 | 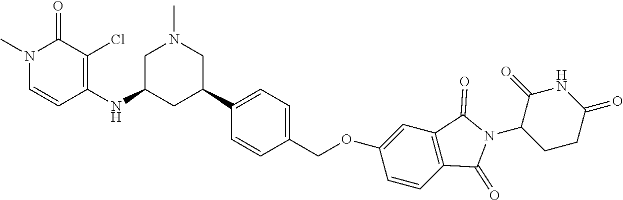 [SMILES: CN1C[C@H](Nc2ccn(C)c(=O)c2Cl)C[C@H](c2ccc(COc3ccc4c(c3)C(=O)N(C3CCC(=O)NC3=O)C4=O)cc2)C1] |
| 105a | 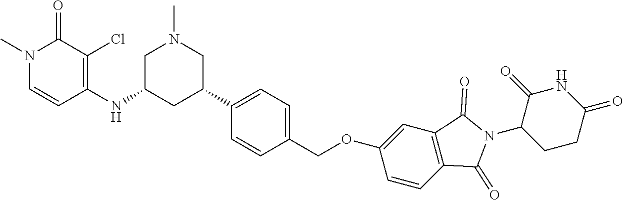 [SMILES: CN1C[C@@H](Nc2ccn(C)c(=O)c2Cl)C[C@@H](c2ccc(COc3ccc4c(c3)C(=O)N(C3CCC(=O)NC3=O)C4=O)cc2)C1] |
| 105b | 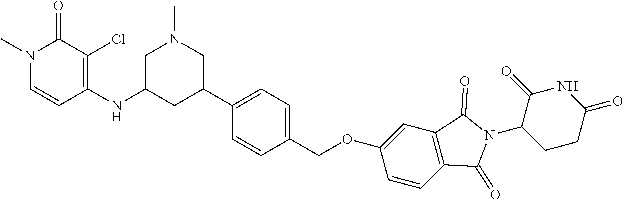 [SMILES: CN1CC(Nc2ccn(C)c(=O)c2Cl)CC(c2ccc(COc3ccc4c(c3)C(=O)N(C3CCC(=O)NC3=O)C4=O)cc2)C1] |
| 106 | 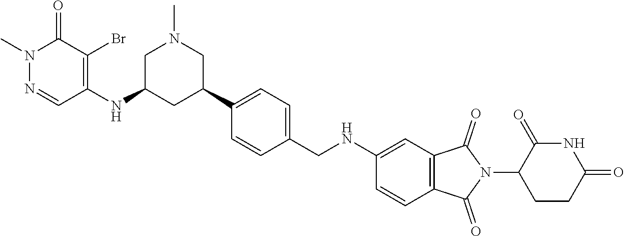 [SMILES: CN1C[C@H](Nc2cnn(C)c(=O)c2Br)C[C@H](c2ccc(CNc3ccc4c(c3)C(=O)N(C3CCC(=O)NC3=O)C4=O)cc2)C1] |
| 106a | 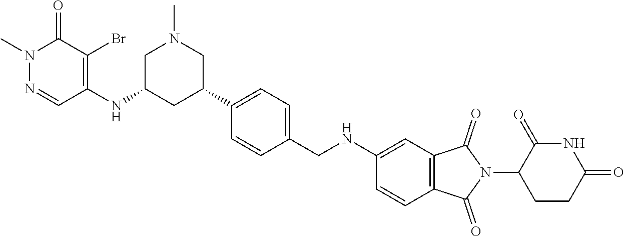 [SMILES: CN1C[C@@H](Nc2cnn(C)c(=O)c2Br)C[C@@H](c2ccc(CNc3ccc4c(c3)C(=O)N(C3CCC(=O)NC3=O)C4=O)cc2)C1] |
| 106b | 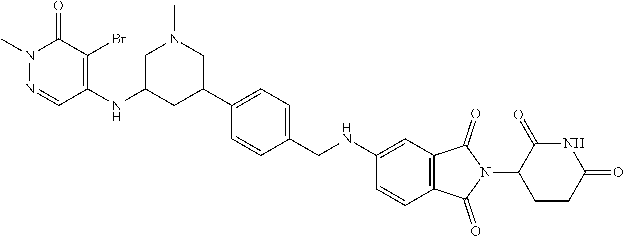 [SMILES: CN1CC(Nc2cnn(C)c(=O)c2Br)CC(c2ccc(CNc3ccc4c(c3)C(=O)N(C3CCC(=O)NC3=O)C4=O)cc2)C1] |
| 107 | 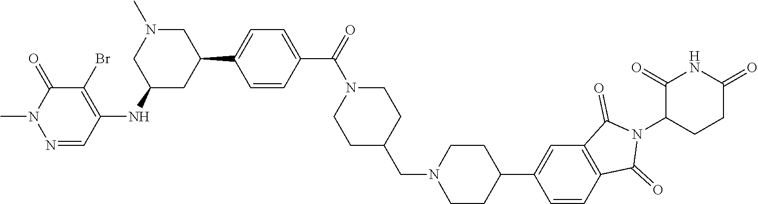 [SMILES: CN1C[C@H](Nc2cnn(C)c(=O)c2Br)C[C@H](c2ccc(C(=O)N3CCC(CN4CCC(c5ccc6c(c5)C(=O)N(C5CCC(=O)NC5=O)C6=O)CC4)CC3)cc2)C1] |
| 107a | 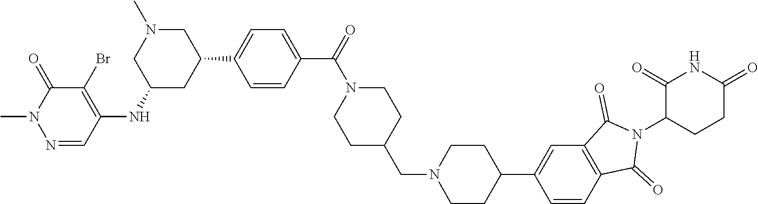 [SMILES: CN1C[C@@H](Nc2cnn(C)c(=O)c2Br)C[C@@H](c2ccc(C(=O)N3CCC(CN4CCC(c5ccc6c(c5)C(=O)N(C5CCC(=O)NC5=O)C6=O)CC4)CC3)cc2)C1] |
| 107b | 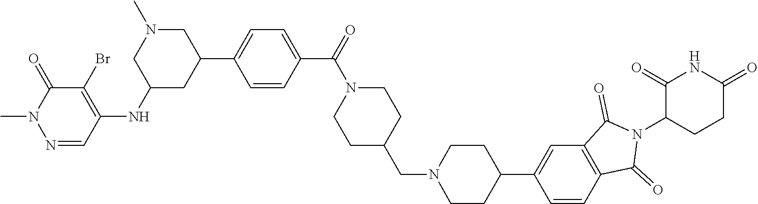 [SMILES: CN1CC(Nc2cnn(C)c(=O)c2Br)CC(c2ccc(C(=O)N3CCC(CN4CCC(c5ccc6c(c5)C(=O)N(C5CCC(=O)NC5=O)C6=O)CC4)CC3)cc2)C1] |
| 108 | 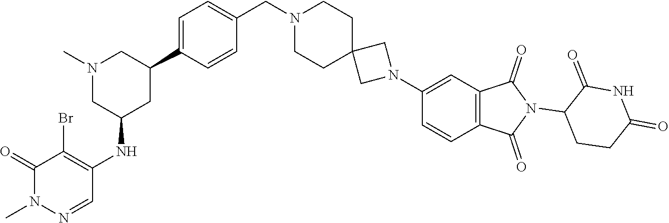 [SMILES: CN1C[C@H](Nc2cnn(C)c(=O)c2Br)C[C@H](c2ccc(CN3CCC4(CC3)CN(c3ccc5c(c3)C(=O)N(C3CCC(=O)NC3=O)C5=O)C4)cc2)C1] |
| 108a | 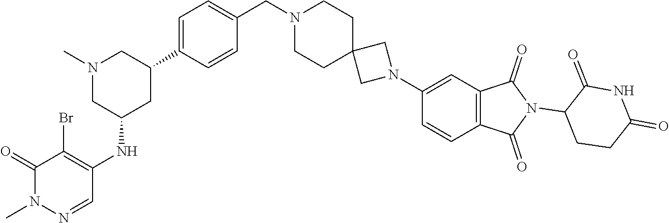 [SMILES: CN1C[C@@H](Nc2cnn(C)c(=O)c2Br)C[C@@H](c2ccc(CN3CCC4(CC3)CN(c3ccc5c(c3)C(=O)N(C3CCC(=O)NC3=O)C5=O)C4)cc2)C1] |
| 108b | 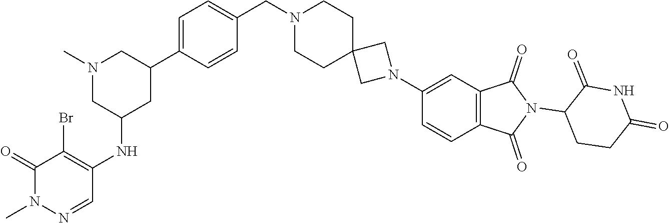 [SMILES: CN1CC(Nc2cnn(C)c(=O)c2Br)CC(c2ccc(CN3CCC4(CC3)CN(c3ccc5c(c3)C(=O)N(C3CCC(=O)NC3=O)C5=O)C4)cc2)C1] |
| 109 | 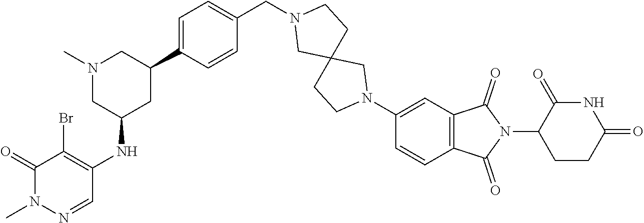 [SMILES: CN1C[C@H](Nc2cnn(C)c(=O)c2Br)C[C@H](c2ccc(CN3CCC4(CCN(c5ccc6c(c5)C(=O)N(C5CCC(=O)NC5=O)C6=O)C4)C3)cc2)C1] |
| 109a | 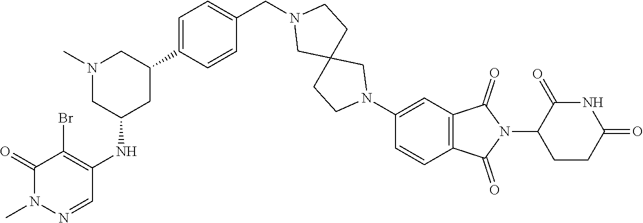 [SMILES: CN1C[C@@H](Nc2cnn(C)c(=O)c2Br)C[C@@H](c2ccc(CN3CCC4(CCN(c5ccc6c(c5)C(=O)N(C5CCC(=O)NC5=O)C6=O)C4)C3)cc2)C1] |
| 109b | 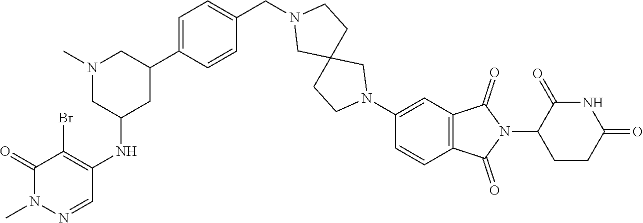 [SMILES: CN1CC(Nc2cnn(C)c(=O)c2Br)CC(c2ccc(CN3CCC4(CCN(c5ccc6c(c5)C(=O)N(C5CCC(=O)NC5=O)C6=O)C4)C3)cc2)C1] |
| 110 | 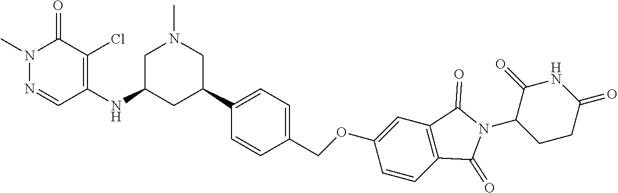 [SMILES: CN1C[C@H](Nc2cnn(C)c(=O)c2Cl)C[C@H](c2ccc(COc3ccc4c(c3)C(=O)N(C3CCC(=O)NC3=O)C4=O)cc2)C1] |
| 110a | 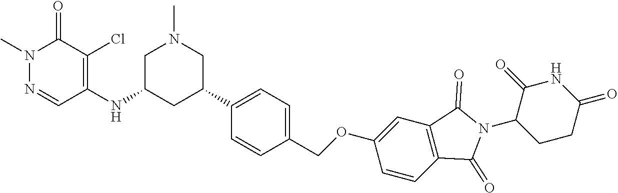 [SMILES: CN1C[C@@H](Nc2cnn(C)c(=O)c2Cl)C[C@@H](c2ccc(COc3ccc4c(c3)C(=O)N(C3CCC(=O)NC3=O)C4=O)cc2)C1] |
| 110b | 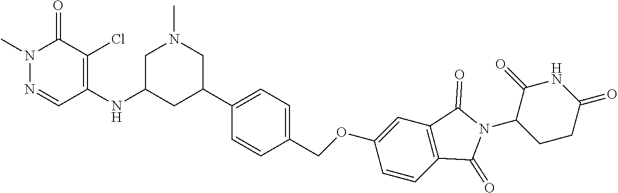 [SMILES: CN1CC(Nc2cnn(C)c(=O)c2Cl)CC(c2ccc(COc3ccc4c(c3)C(=O)N(C3CCC(=O)NC3=O)C4=O)cc2)C1] |
| 111 | 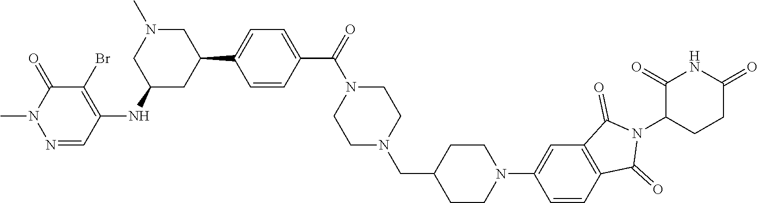 [SMILES: CN1C[C@H](Nc2cnn(C)c(=O)c2Br)C[C@H](c2ccc(C(=O)N3CCN(CC4CCN(c5ccc6c(c5)C(=O)N(C5CCC(=O)NC5=O)C6=O)CC4)CC3)cc2)C1] |
| 111a | 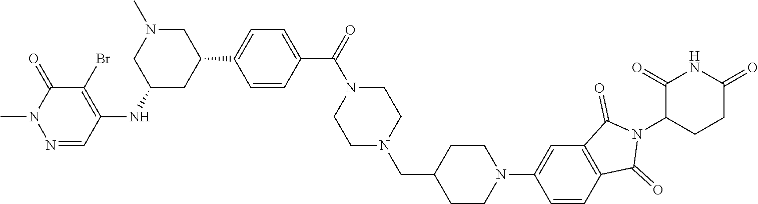 [SMILES: CN1C[C@@H](Nc2cnn(C)c(=O)c2Br)C[C@@H](c2ccc(C(=O)N3CCN(CC4CCN(c5ccc6c(c5)C(=O)N(C5CCC(=O)NC5=O)C6=O)CC4)CC3)cc2)C1] |
| 111b | 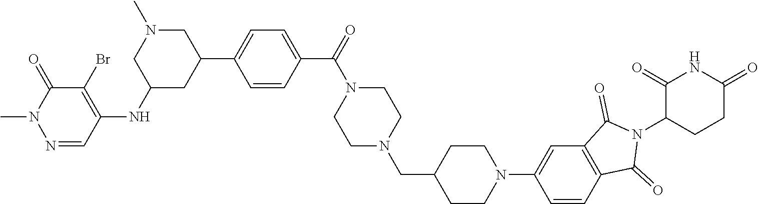 [SMILES: CN1CC(Nc2cnn(C)c(=O)c2Br)CC(c2ccc(C(=O)N3CCN(CC4CCN(c5ccc6c(c5)C(=O)N(C5CCC(=O)NC5=O)C6=O)CC4)CC3)cc2)C1] |
| 112 | 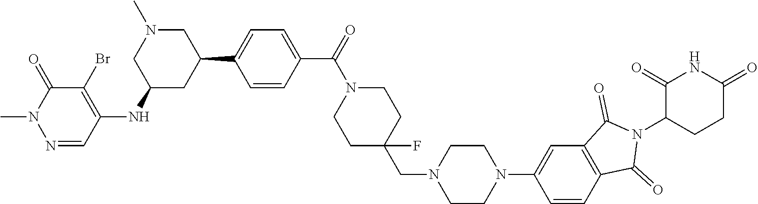 [SMILES: CN1C[C@H](Nc2cnn(C)c(=O)c2Br)C[C@H](c2ccc(C(=O)N3CCC(F)(CN4CCN(c5ccc6c(c5)C(=O)N(C5CCC(=O)NC5=O)C6=O)CC4)CC3)cc2)C1] |
| 112a | 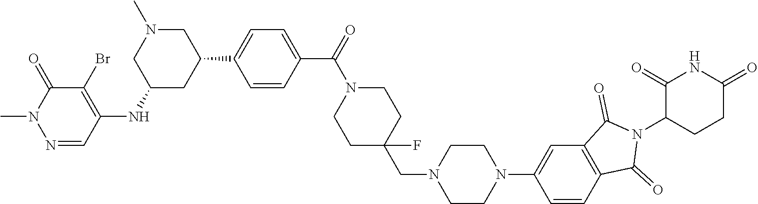 [SMILES: CN1C[C@@H](Nc2cnn(C)c(=O)c2Br)C[C@@H](c2ccc(C(=O)N3CCC(F)(CN4CCN(c5ccc6c(c5)C(=O)N(C5CCC(=O)NC5=O)C6=O)CC4)CC3)cc2)C1] |
| 112b | 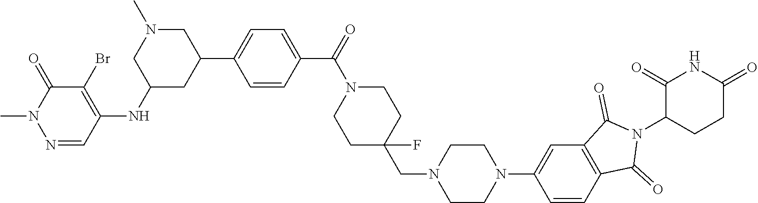 [SMILES: CN1CC(Nc2cnn(C)c(=O)c2Br)CC(c2ccc(C(=O)N3CCC(F)(CN4CCN(c5ccc6c(c5)C(=O)N(C5CCC(=O)NC5=O)C6=O)CC4)CC3)cc2)C1] |
| 113 | 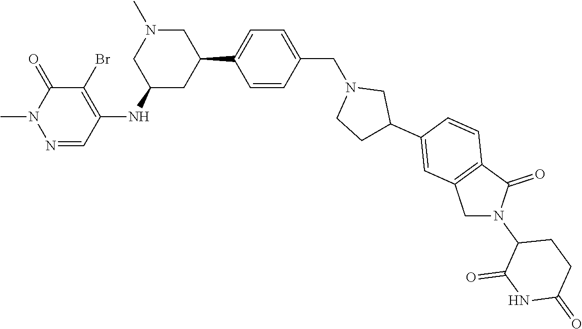 [SMILES: CN1C[C@H](Nc2cnn(C)c(=O)c2Br)C[C@H](c2ccc(CN3CCC(c4ccc5c(c4)CN(C4CCC(=O)NC4=O)C5=O)C3)cc2)C1] |
| 113a | 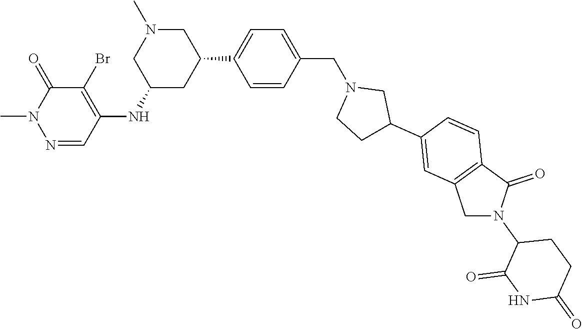 [SMILES: CN1C[C@@H](Nc2cnn(C)c(=O)c2Br)C[C@@H](c2ccc(CN3CCC(c4ccc5c(c4)CN(C4CCC(=O)NC4=O)C5=O)C3)cc2)C1] |
| 113b | 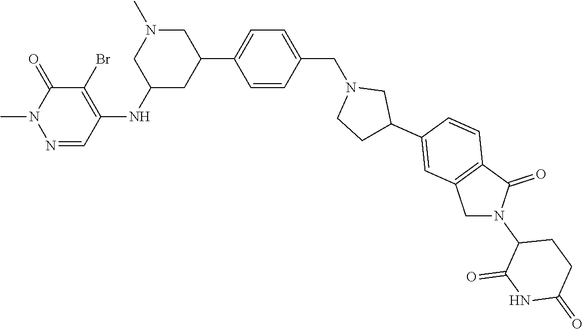 [SMILES: CN1CC(Nc2cnn(C)c(=O)c2Br)CC(c2ccc(CN3CCC(c4ccc5c(c4)CN(C4CCC(=O)NC4=O)C5=O)C3)cc2)C1] |
| 114 | 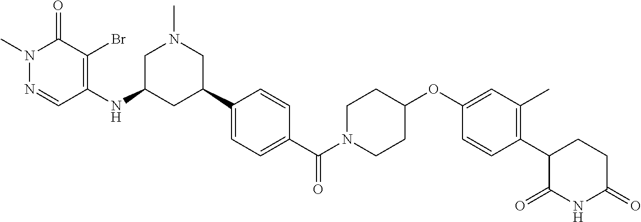 [SMILES: Cc1cc(OC2CCN(C(=O)c3ccc([C@H]4C[C@@H](Nc5cnn(C)c(=O)c5Br)CN(C)C4)cc3)CC2)ccc1C1CCC(=O)NC1=O] |
| 114a | 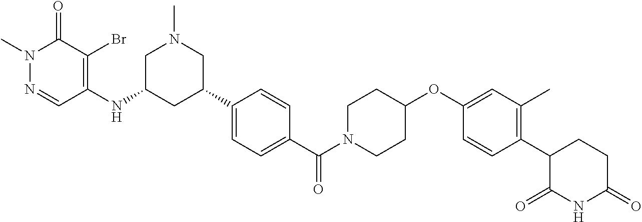 [SMILES: Cc1cc(OC2CCN(C(=O)c3ccc([C@@H]4C[C@H](Nc5cnn(C)c(=O)c5Br)CN(C)C4)cc3)CC2)ccc1C1CCC(=O)NC1=O] |
| 114b | 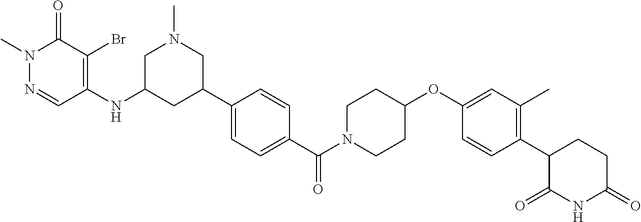 [SMILES: Cc1cc(OC2CCN(C(=O)c3ccc(C4CC(Nc5cnn(C)c(=O)c5Br)CN(C)C4)cc3)CC2)ccc1C1CCC(=O)NC1=O] |
| 115 | 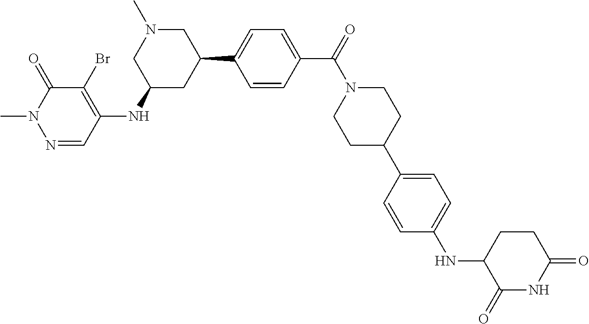 [SMILES: CN1C[C@H](Nc2cnn(C)c(=O)c2Br)C[C@H](c2ccc(C(=O)N3CCC(c4ccc(NC5CCC(=O)NC5=O)cc4)CC3)cc2)C1] |
| 115a | 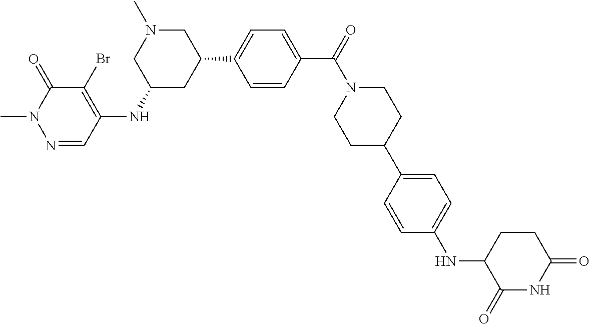 [SMILES: CN1C[C@@H](Nc2cnn(C)c(=O)c2Br)C[C@@H](c2ccc(C(=O)N3CCC(c4ccc(NC5CCC(=O)NC5=O)cc4)CC3)cc2)C1] |
| 115b | 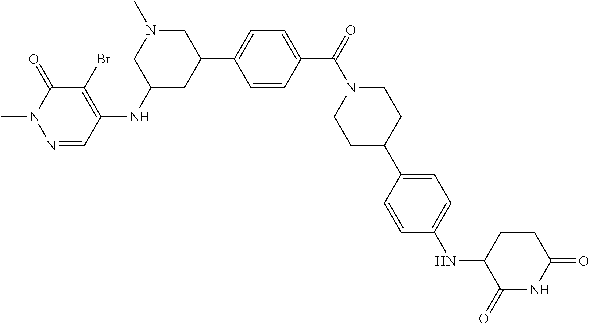 [SMILES: CN1CC(Nc2cnn(C)c(=O)c2Br)CC(c2ccc(C(=O)N3CCC(c4ccc(NC5CCC(=O)NC5=O)cc4)CC3)cc2)C1] |
| 116 | 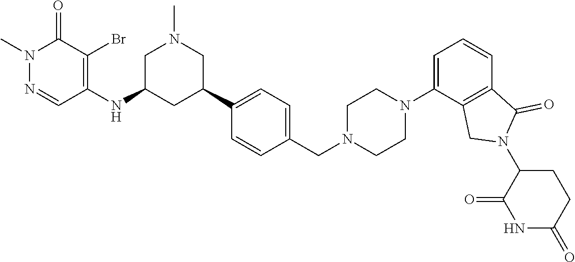 [SMILES: CN1C[C@H](Nc2cnn(C)c(=O)c2Br)C[C@H](c2ccc(CN3CCN(c4cccc5c4CN(C4CCC(=O)NC4=O)C5=O)CC3)cc2)C1] |
| 116a | 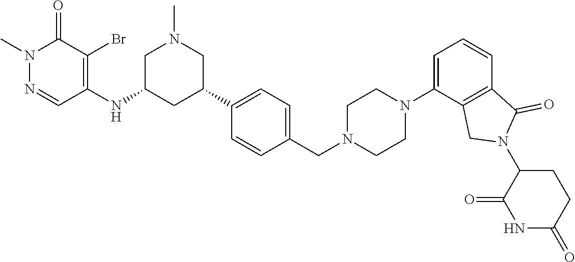 [SMILES: CN1C[C@@H](Nc2cnn(C)c(=O)c2Br)C[C@@H](c2ccc(CN3CCN(c4cccc5c4CN(C4CCC(=O)NC4=O)C5=O)CC3)cc2)C1] |
| 116b | 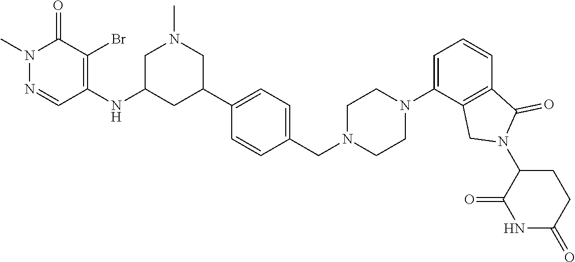 [SMILES: CN1CC(Nc2cnn(C)c(=O)c2Br)CC(c2ccc(CN3CCN(c4cccc5c4CN(C4CCC(=O)NC4=O)C5=O)CC3)cc2)C1] |
| 117 | 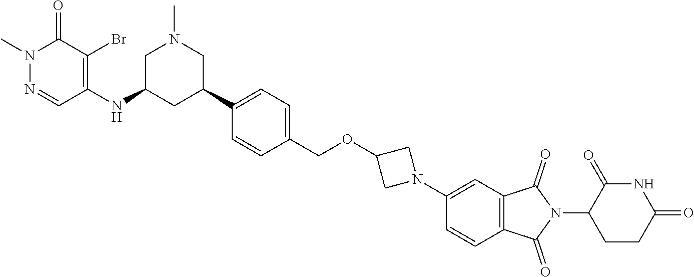 [SMILES: CN1C[C@H](Nc2cnn(C)c(=O)c2Br)C[C@H](c2ccc(COC3CN(c4ccc5c(c4)C(=O)N(C4CCC(=O)NC4=O)C5=O)C3)cc2)C1] |
| 117a | 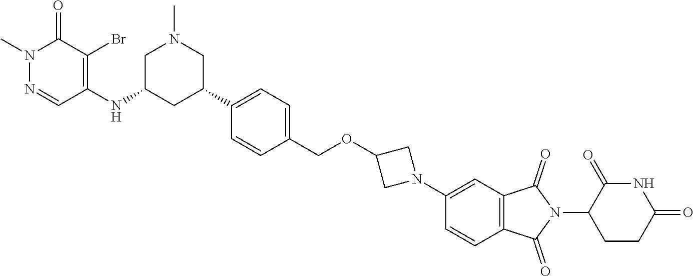 [SMILES: CN1C[C@@H](Nc2cnn(C)c(=O)c2Br)C[C@@H](c2ccc(COC3CN(c4ccc5c(c4)C(=O)N(C4CCC(=O)NC4=O)C5=O)C3)cc2)C1] |
| 117b | 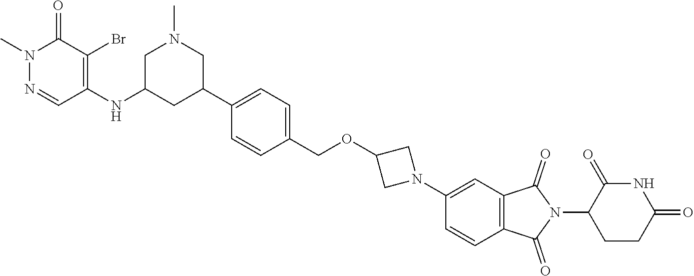 [SMILES: CN1CC(Nc2cnn(C)c(=O)c2Br)CC(c2ccc(COC3CN(c4ccc5c(c4)C(=O)N(C4CCC(=O)NC4=O)C5=O)C3)cc2)C1] |
| 118 | 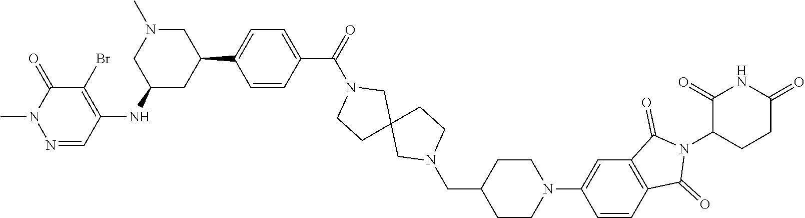 [SMILES: CN1C[C@H](Nc2cnn(C)c(=O)c2Br)C[C@H](c2ccc(C(=O)N3CCC4(CCN(CC5CCN(c6ccc7c(c6)C(=O)N(C6CCC(=O)NC6=O)C7=O)CC5)C4)C3)cc2)C1] |
| 118a | 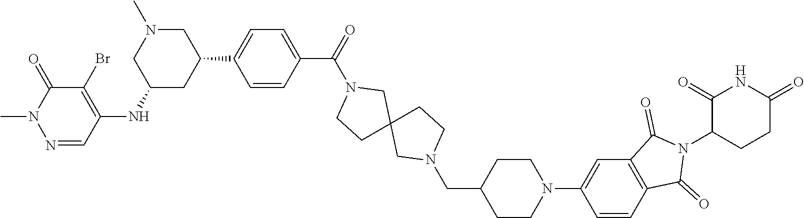 [SMILES: CN1C[C@@H](Nc2cnn(C)c(=O)c2Br)C[C@@H](c2ccc(C(=O)N3CCC4(CCN(CC5CCN(c6ccc7c(c6)C(=O)N(C6CCC(=O)NC6=O)C7=O)CC5)C4)C3)cc2)C1] |
| 118b | 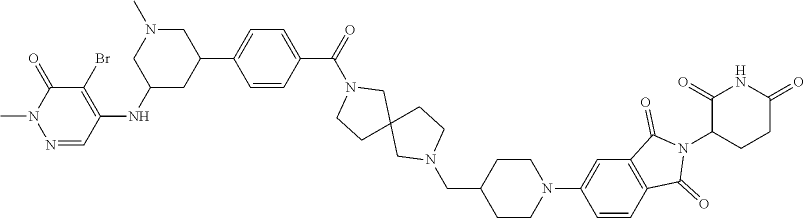 [SMILES: CN1CC(Nc2cnn(C)c(=O)c2Br)CC(c2ccc(C(=O)N3CCC4(CCN(CC5CCN(c6ccc7c(c6)C(=O)N(C6CCC(=O)NC6=O)C7=O)CC5)C4)C3)cc2)C1] |
| 119 | 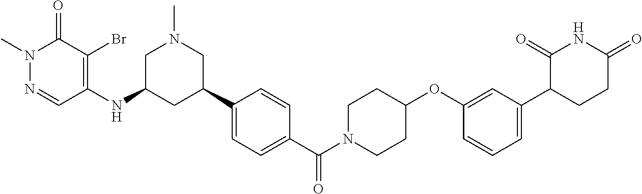 [SMILES: CN1C[C@H](Nc2cnn(C)c(=O)c2Br)C[C@H](c2ccc(C(=O)N3CCC(Oc4cccc(C5CCC(=O)NC5=O)c4)CC3)cc2)C1] |
| 119a | 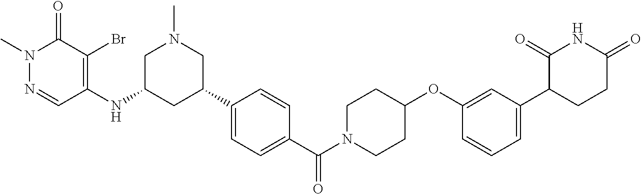 [SMILES: CN1C[C@@H](Nc2cnn(C)c(=O)c2Br)C[C@@H](c2ccc(C(=O)N3CCC(Oc4cccc(C5CCC(=O)NC5=O)c4)CC3)cc2)C1] |
| 119b | 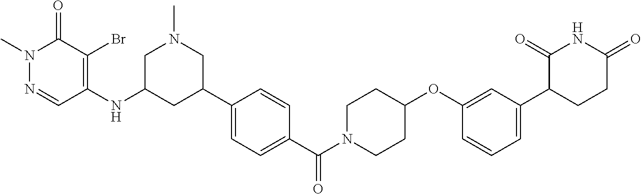 [SMILES: CN1CC(Nc2cnn(C)c(=O)c2Br)CC(c2ccc(C(=O)N3CCC(Oc4cccc(C5CCC(=O)NC5=O)c4)CC3)cc2)C1] |
| 120 | 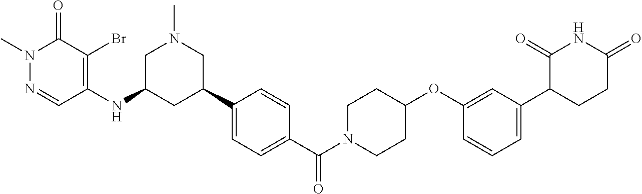 [SMILES: CN1C[C@H](Nc2cnn(C)c(=O)c2Br)C[C@H](c2ccc(C(=O)N3CCC(Oc4cccc(C5CCC(=O)NC5=O)c4)CC3)cc2)C1] |
| 120a | 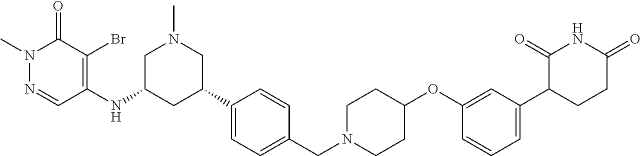 [SMILES: CN1C[C@@H](Nc2cnn(C)c(=O)c2Br)C[C@@H](c2ccc(CN3CCC(Oc4cccc(C5CCC(=O)NC5=O)c4)CC3)cc2)C1] |
| 120b | 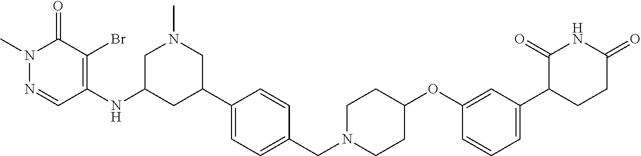 [SMILES: CN1CC(Nc2cnn(C)c(=O)c2Br)CC(c2ccc(CN3CCC(Oc4cccc(C5CCC(=O)NC5=O)c4)CC3)cc2)C1] |
| 121 | 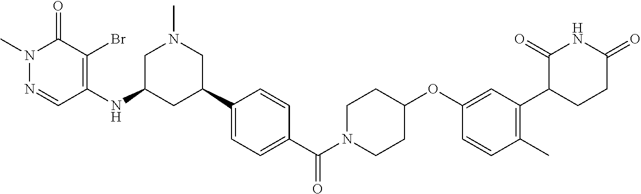 [SMILES: Cc1ccc(OC2CCN(C(=O)c3ccc([C@H]4C[C@@H](Nc5cnn(C)c(=O)c5Br)CN(C)C4)cc3)CC2)cc1C1CCC(=O)NC1=O] |
| 121a | 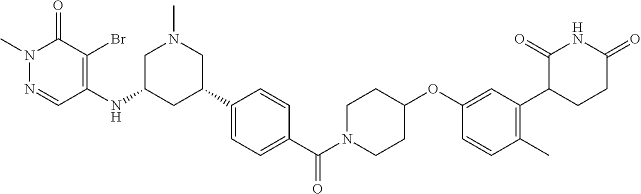 [SMILES: Cc1ccc(OC2CCN(C(=O)c3ccc([C@@H]4C[C@H](Nc5cnn(C)c(=O)c5Br)CN(C)C4)cc3)CC2)cc1C1CCC(=O)NC1=O] |
| 121b | 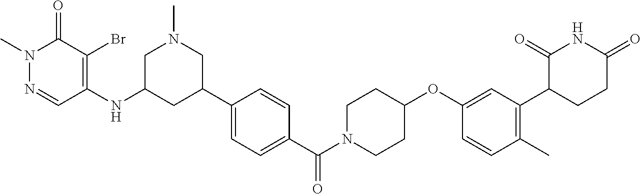 [SMILES: Cc1ccc(OC2CCN(C(=O)c3ccc(C4CC(Nc5cnn(C)c(=O)c5Br)CN(C)C4)cc3)CC2)cc1C1CCC(=O)NC1=O] |
| 122 | 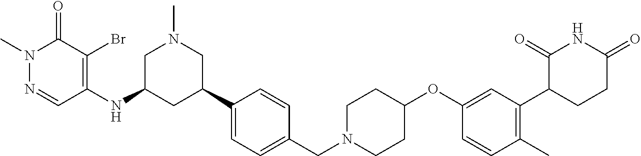 [SMILES: Cc1ccc(OC2CCN(Cc3ccc([C@H]4C[C@@H](Nc5cnn(C)c(=O)c5Br)CN(C)C4)cc3)CC2)cc1C1CCC(=O)NC1=O] |
| 122a | 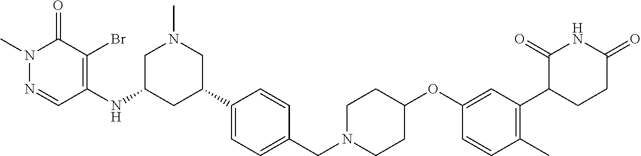 [SMILES: Cc1ccc(OC2CCN(Cc3ccc([C@@H]4C[C@H](Nc5cnn(C)c(=O)c5Br)CN(C)C4)cc3)CC2)cc1C1CCC(=O)NC1=O] |
| 122b | 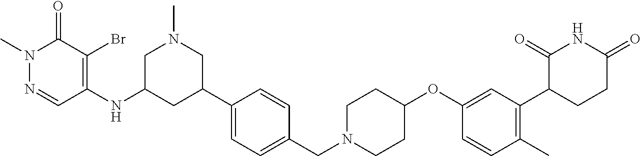 [SMILES: Cc1ccc(OC2CCN(Cc3ccc(C4CC(Nc5cnn(C)c(=O)c5Br)CN(C)C4)cc3)CC2)cc1C1CCC(=O)NC1=O] |
| 123 | 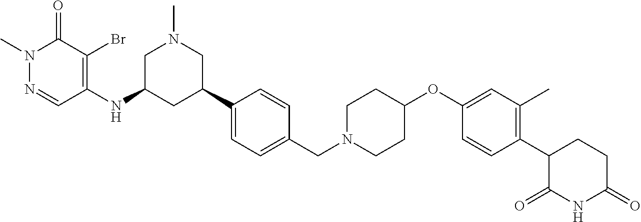 [SMILES: Cc1cc(OC2CCN(Cc3ccc([C@H]4C[C@@H](Nc5cnn(C)c(=O)c5Br)CN(C)C4)cc3)CC2)ccc1C1CCC(=O)NC1=O] |
| 123a | 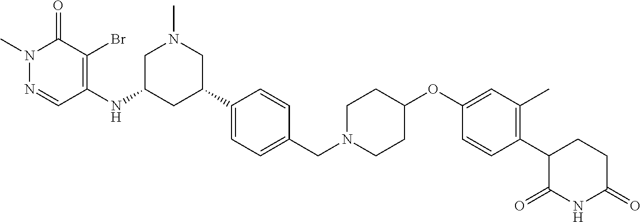 [SMILES: Cc1cc(OC2CCN(Cc3ccc([C@@H]4C[C@H](Nc5cnn(C)c(=O)c5Br)CN(C)C4)cc3)CC2)ccc1C1CCC(=O)NC1=O] |
| 123b | 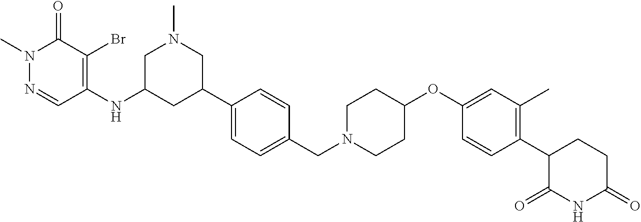 [SMILES: Cc1cc(OC2CCN(Cc3ccc(C4CC(Nc5cnn(C)c(=O)c5Br)CN(C)C4)cc3)CC2)ccc1C1CCC(=O)NC1=O] |
| 124 | 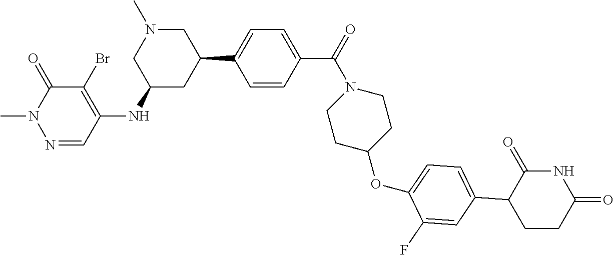 [SMILES: CN1C[C@H](Nc2cnn(C)c(=O)c2Br)C[C@H](c2ccc(C(=O)N3CCC(Oc4ccc(C5CCC(=O)NC5=O)cc4F)CC3)cc2)C1] |
| 124a | 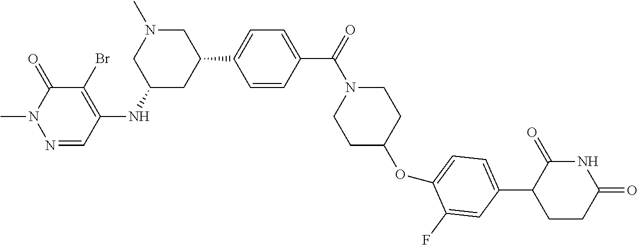 [SMILES: CN1C[C@@H](Nc2cnn(C)c(=O)c2Br)C[C@@H](c2ccc(C(=O)N3CCC(Oc4ccc(C5CCC(=O)NC5=O)cc4F)CC3)cc2)C1] |
| 124b | 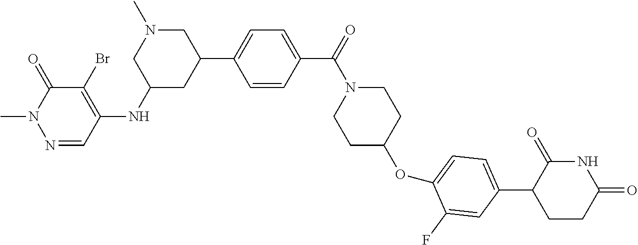 [SMILES: CN1CC(Nc2cnn(C)c(=O)c2Br)CC(c2ccc(C(=O)N3CCC(Oc4ccc(C5CCC(=O)NC5=O)cc4F)CC3)cc2)C1] |
| 125 | 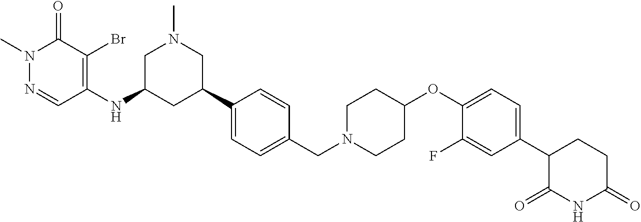 [SMILES: CN1C[C@H](Nc2cnn(C)c(=O)c2Br)C[C@H](c2ccc(CN3CCC(Oc4ccc(C5CCC(=O)NC5=O)cc4F)CC3)cc2)C1] |
| 125a | 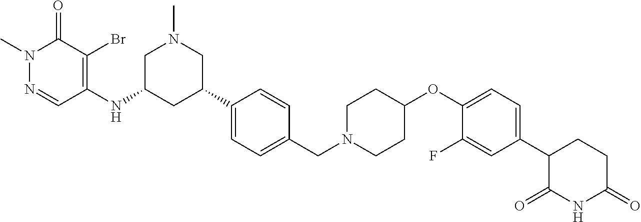 [SMILES: CN1C[C@@H](Nc2cnn(C)c(=O)c2Br)C[C@@H](c2ccc(CN3CCC(Oc4ccc(C5CCC(=O)NC5=O)cc4F)CC3)cc2)C1] |
| 125b | 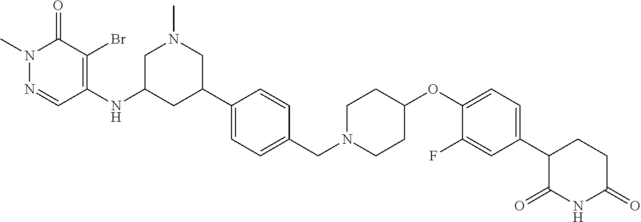 [SMILES: CN1CC(Nc2cnn(C)c(=O)c2Br)CC(c2ccc(CN3CCC(Oc4ccc(C5CCC(=O)NC5=O)cc4F)CC3)cc2)C1] |
| 126 | 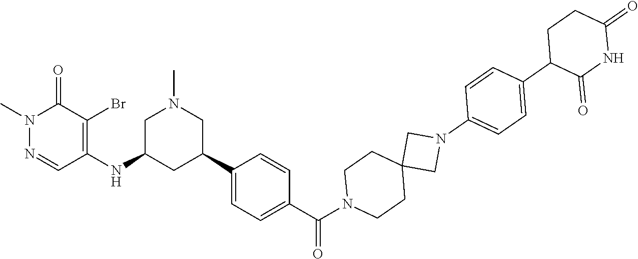 [SMILES: CN1C[C@H](Nc2cnn(C)c(=O)c2Br)C[C@H](c2ccc(C(=O)N3CCC4(CC3)CN(c3ccc(C5CCC(=O)NC5=O)cc3)C4)cc2)C1] |
| 126a | 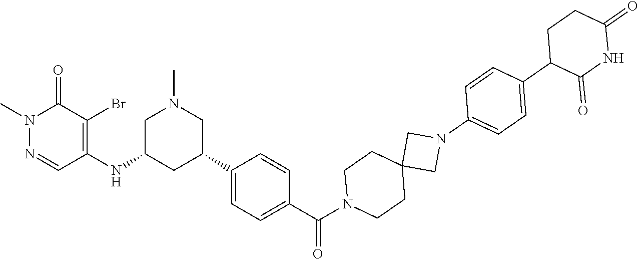 [SMILES: CN1C[C@@H](Nc2cnn(C)c(=O)c2Br)C[C@@H](c2ccc(C(=O)N3CCC4(CC3)CN(c3ccc(C5CCC(=O)NC5=O)cc3)C4)cc2)C1] |
| 126b | 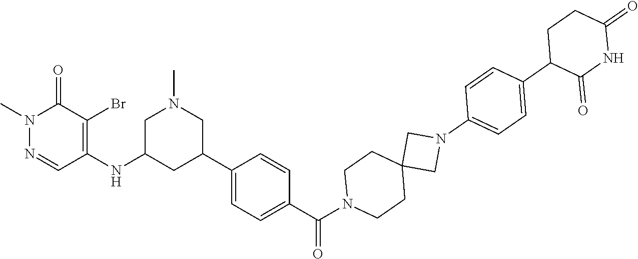 [SMILES: CN1CC(Nc2cnn(C)c(=O)c2Br)CC(c2ccc(C(=O)N3CCC4(CC3)CN(c3ccc(C5CCC(=O)NC5=O)cc3)C4)cc2)C1] |
| 127 | 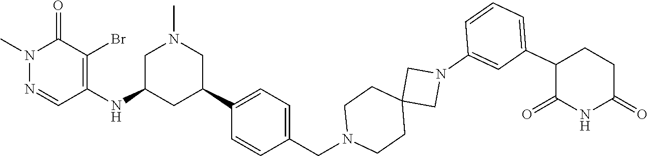 [SMILES: CN1C[C@H](Nc2cnn(C)c(=O)c2Br)C[C@H](c2ccc(CN3CCC4(CC3)CN(c3cccc(C5CCC(=O)NC5=O)c3)C4)cc2)C1] |
| 127a | 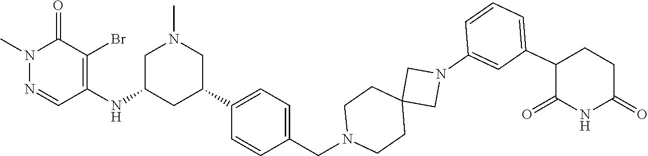 [SMILES: CN1C[C@@H](Nc2cnn(C)c(=O)c2Br)C[C@@H](c2ccc(CN3CCC4(CC3)CN(c3cccc(C5CCC(=O)NC5=O)c3)C4)cc2)C1] |
| 127b | 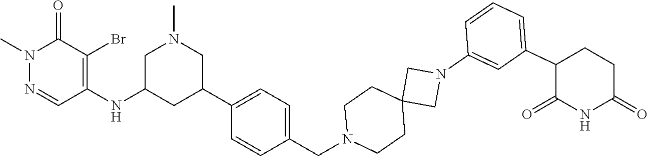 [SMILES: CN1CC(Nc2cnn(C)c(=O)c2Br)CC(c2ccc(CN3CCC4(CC3)CN(c3cccc(C5CCC(=O)NC5=O)c3)C4)cc2)C1] |
| 128 | 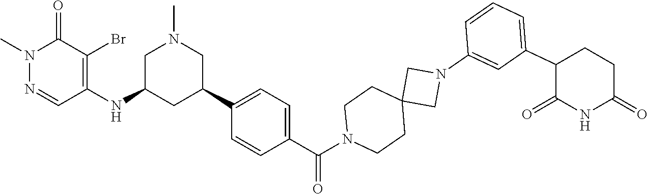 [SMILES: CN1C[C@H](Nc2cnn(C)c(=O)c2Br)C[C@H](c2ccc(C(=O)N3CCC4(CC3)CN(c3cccc(C5CCC(=O)NC5=O)c3)C4)cc2)C1] |
| 128a | 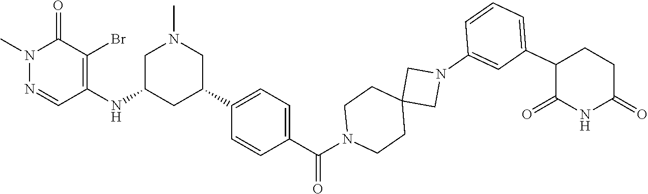 [SMILES: CN1C[C@@H](Nc2cnn(C)c(=O)c2Br)C[C@@H](c2ccc(C(=O)N3CCC4(CC3)CN(c3cccc(C5CCC(=O)NC5=O)c3)C4)cc2)C1] |
| 128b | 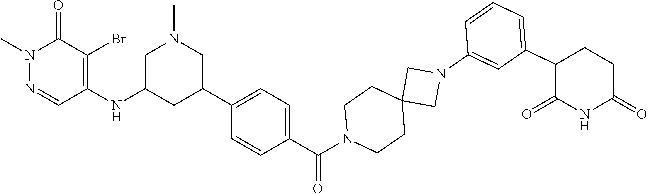 [SMILES: CN1CC(Nc2cnn(C)c(=O)c2Br)CC(c2ccc(C(=O)N3CCC4(CC3)CN(c3cccc(C5CCC(=O)NC5=O)c3)C4)cc2)C1] |
| 129 | 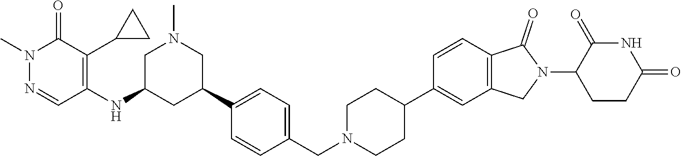 [SMILES: CN1C[C@H](Nc2cnn(C)c(=O)c2C2CC2)C[C@H](c2ccc(CN3CCC(c4ccc5c(c4)CN(C4CCC(=O)NC4=O)C5=O)CC3)cc2)C1] |
| 129a | 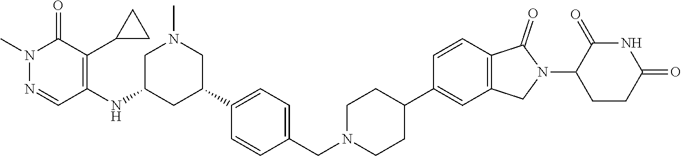 [SMILES: CN1C[C@@H](Nc2cnn(C)c(=O)c2C2CC2)C[C@@H](c2ccc(CN3CCC(c4ccc5c(c4)CN(C4CCC(=O)NC4=O)C5=O)CC3)cc2)C1] |
| 129b | 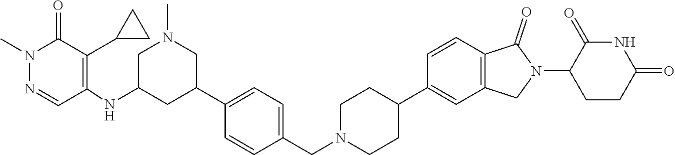 [SMILES: CN1CC(Nc2cnn(C)c(=O)c2C2CC2)CC(c2ccc(CN3CCC(c4ccc5c(c4)CN(C4CCC(=O)NC4=O)C5=O)CC3)cc2)C1] |
| 130 | 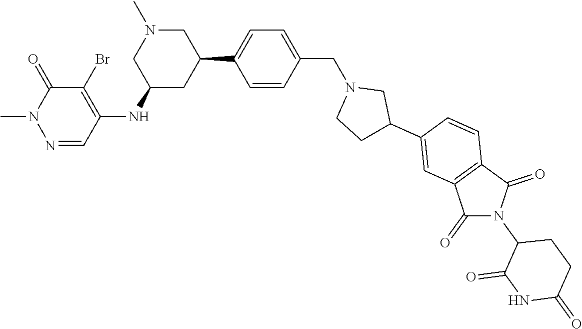 [SMILES: CN1C[C@H](Nc2cnn(C)c(=O)c2Br)C[C@H](c2ccc(CN3CCC(c4ccc5c(c4)C(=O)N(C4CCC(=O)NC4=O)C5=O)C3)cc2)C1] |
| 130a | 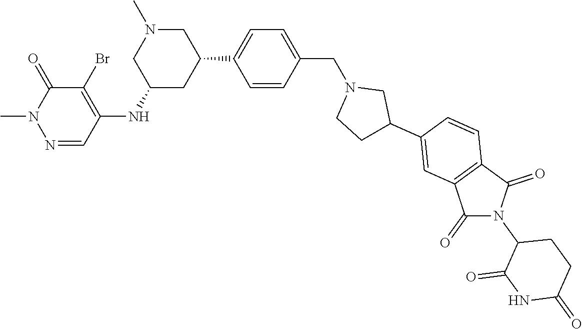 [SMILES: CN1C[C@@H](Nc2cnn(C)c(=O)c2Br)C[C@@H](c2ccc(CN3CCC(c4ccc5c(c4)C(=O)N(C4CCC(=O)NC4=O)C5=O)C3)cc2)C1] |
| 130b | 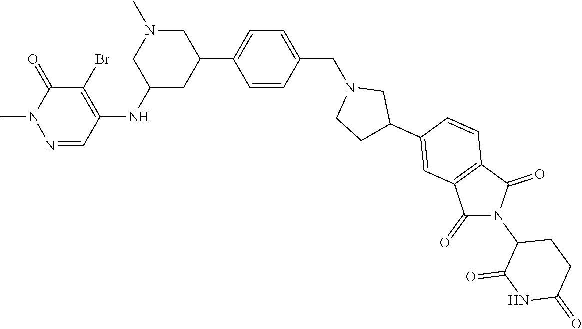 [SMILES: CN1CC(Nc2cnn(C)c(=O)c2Br)CC(c2ccc(CN3CCC(c4ccc5c(c4)C(=O)N(C4CCC(=O)NC4=O)C5=O)C3)cc2)C1] |
| 131 | 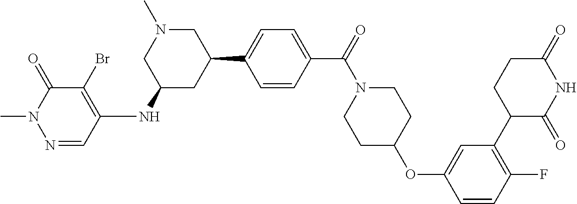 [SMILES: CN1C[C@H](Nc2cnn(C)c(=O)c2Br)C[C@H](c2ccc(C(=O)N3CCC(Oc4ccc(F)c(C5CCC(=O)NC5=O)c4)CC3)cc2)C1] |
| 131a | 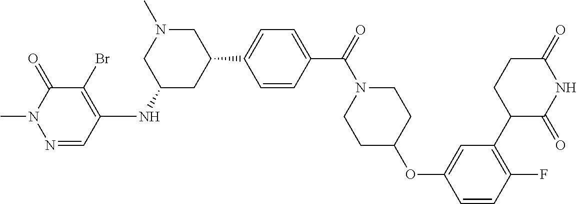 [SMILES: CN1C[C@@H](Nc2cnn(C)c(=O)c2Br)C[C@@H](c2ccc(C(=O)N3CCC(Oc4ccc(F)c(C5CCC(=O)NC5=O)c4)CC3)cc2)C1] |
| 131b | 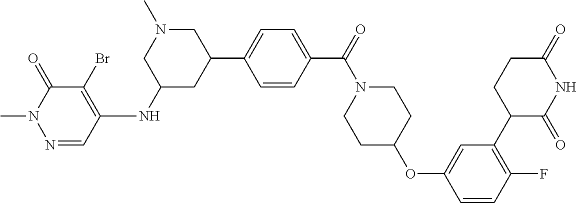 [SMILES: CN1CC(Nc2cnn(C)c(=O)c2Br)CC(c2ccc(C(=O)N3CCC(Oc4ccc(F)c(C5CCC(=O)NC5=O)c4)CC3)cc2)C1] |
| 132 | 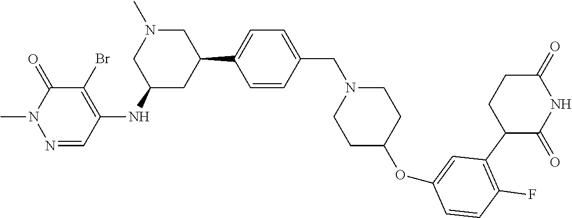 [SMILES: CN1C[C@H](Nc2cnn(C)c(=O)c2Br)C[C@H](c2ccc(CN3CCC(Oc4ccc(F)c(C5CCC(=O)NC5=O)c4)CC3)cc2)C1] |
| 132a | 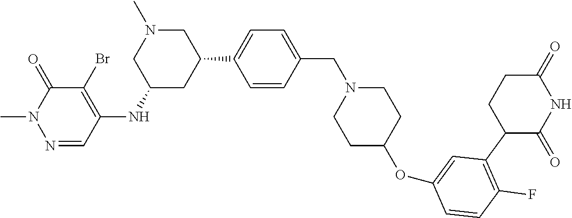 [SMILES: CN1C[C@@H](Nc2cnn(C)c(=O)c2Br)C[C@@H](c2ccc(CN3CCC(Oc4ccc(F)c(C5CCC(=O)NC5=O)c4)CC3)cc2)C1] |
| 132b | 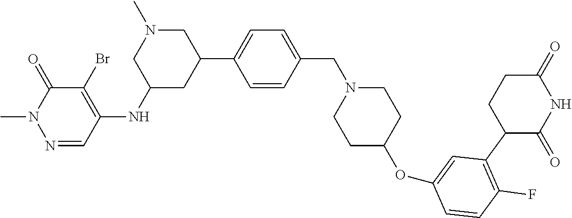 [SMILES: CN1CC(Nc2cnn(C)c(=O)c2Br)CC(c2ccc(CN3CCC(Oc4ccc(F)c(C5CCC(=O)NC5=O)c4)CC3)cc2)C1] |
| 133 | 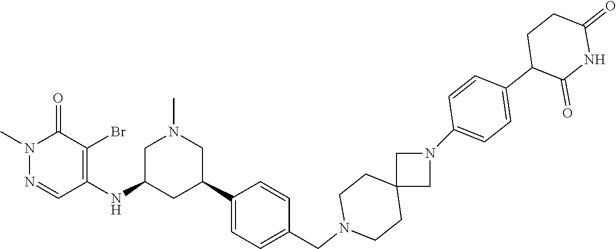 [SMILES: CN1C[C@H](Nc2cnn(C)c(=O)c2Br)C[C@H](c2ccc(CN3CCC4(CC3)CN(c3ccc(C5CCC(=O)NC5=O)cc3)C4)cc2)C1] |
| 133a | 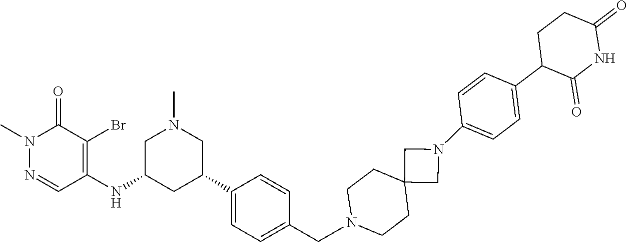 [SMILES: CN1C[C@@H](Nc2cnn(C)c(=O)c2Br)C[C@@H](c2ccc(CN3CCC4(CC3)CN(c3ccc(C5CCC(=O)NC5=O)cc3)C4)cc2)C1] |
| 133b | 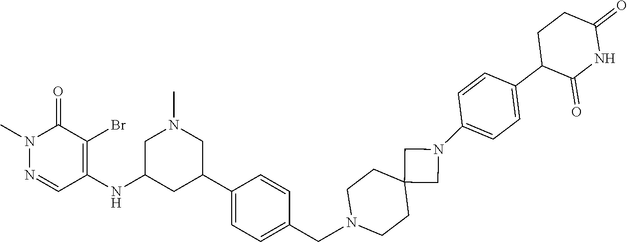 [SMILES: CN1CC(Nc2cnn(C)c(=O)c2Br)CC(c2ccc(CN3CCC4(CC3)CN(c3ccc(C5CCC(=O)NC5=O)cc3)C4)cc2)C1] |
| 134 | 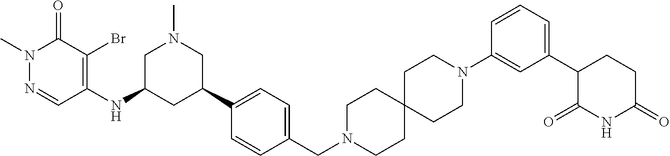 [SMILES: CN1C[C@H](Nc2cnn(C)c(=O)c2Br)C[C@H](c2ccc(CN3CCC4(CC3)CCN(c3cccc(C5CCC(=O)NC5=O)c3)CC4)cc2)C1] |
| 134a | 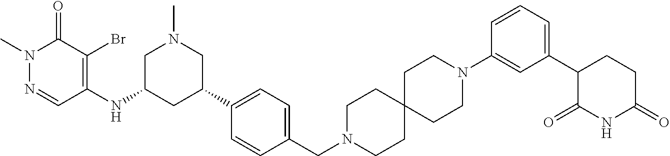 [SMILES: CN1C[C@@H](Nc2cnn(C)c(=O)c2Br)C[C@@H](c2ccc(CN3CCC4(CC3)CCN(c3cccc(C5CCC(=O)NC5=O)c3)CC4)cc2)C1] |
| 134b | 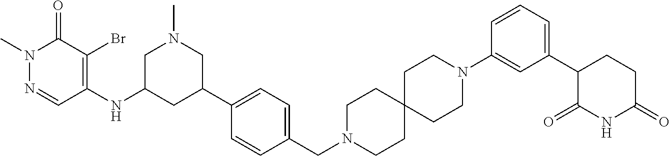 [SMILES: CN1CC(Nc2cnn(C)c(=O)c2Br)CC(c2ccc(CN3CCC4(CC3)CCN(c3cccc(C5CCC(=O)NC5=O)c3)CC4)cc2)C1] |
| 135 | 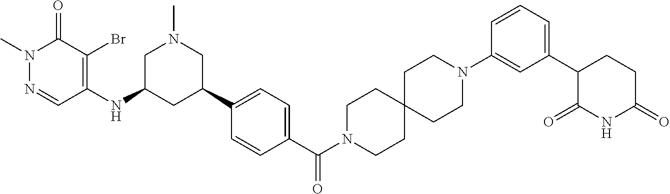 [SMILES: CN1C[C@H](Nc2cnn(C)c(=O)c2Br)C[C@H](c2ccc(C(=O)N3CCC4(CC3)CCN(c3cccc(C5CCC(=O)NC5=O)c3)CC4)cc2)C1] |
| 135a | 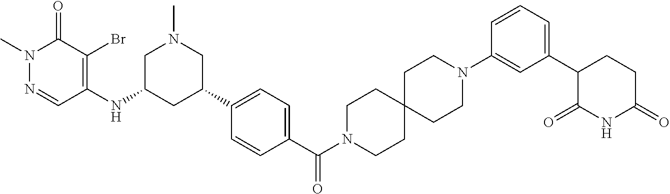 [SMILES: CN1C[C@@H](Nc2cnn(C)c(=O)c2Br)C[C@@H](c2ccc(C(=O)N3CCC4(CC3)CCN(c3cccc(C5CCC(=O)NC5=O)c3)CC4)cc2)C1] |
| 135b | 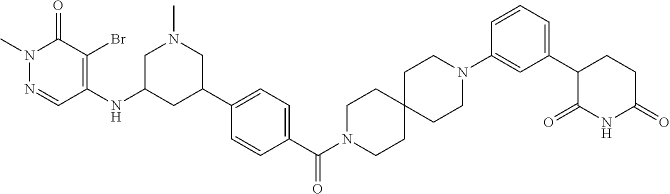 [SMILES: CN1CC(Nc2cnn(C)c(=O)c2Br)CC(c2ccc(C(=O)N3CCC4(CC3)CCN(c3cccc(C5CCC(=O)NC5=O)c3)CC4)cc2)C1] |
| 136 | 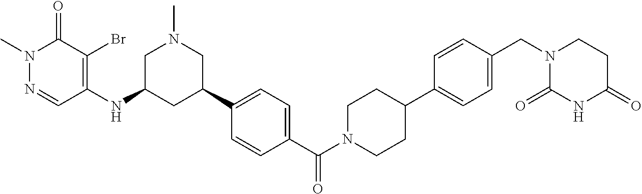 [SMILES: CN1C[C@H](Nc2cnn(C)c(=O)c2Br)C[C@H](c2ccc(C(=O)N3CCC(c4ccc(CN5CCC(=O)NC5=O)cc4)CC3)cc2)C1] |
| 136a | 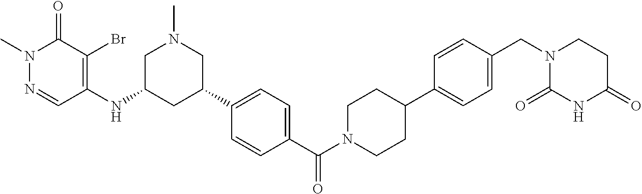 [SMILES: CN1C[C@@H](Nc2cnn(C)c(=O)c2Br)C[C@@H](c2ccc(C(=O)N3CCC(c4ccc(CN5CCC(=O)NC5=O)cc4)CC3)cc2)C1] |
| 136b | 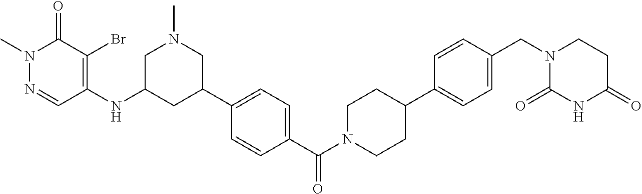 [SMILES: CN1CC(Nc2cnn(C)c(=O)c2Br)CC(c2ccc(C(=O)N3CCC(c4ccc(CN5CCC(=O)NC5=O)cc4)CC3)cc2)C1] |
| 137 | 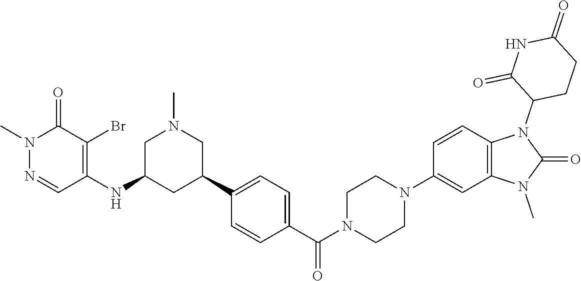 [SMILES: CN1C[C@H](Nc2cnn(C)c(=O)c2Br)C[C@H](c2ccc(C(=O)N3CCN(c4ccc5c(c4)n(C)c(=O)n5C4CCC(=O)NC4=O)CC3)cc2)C1] |
| 137a | 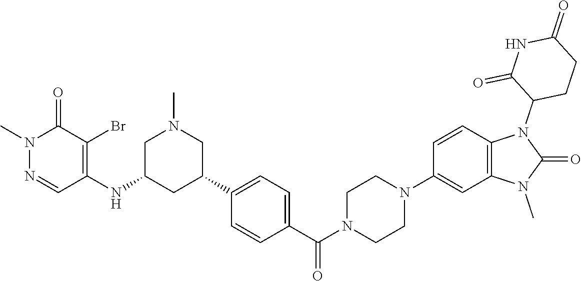 [SMILES: CN1C[C@@H](Nc2cnn(C)c(=O)c2Br)C[C@@H](c2ccc(C(=O)N3CCN(c4ccc5c(c4)n(C)c(=O)n5C4CCC(=O)NC4=O)CC3)cc2)C1] |
| 137b | 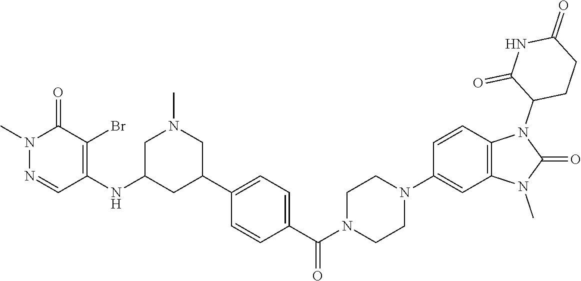 [SMILES: CN1CC(Nc2cnn(C)c(=O)c2Br)CC(c2ccc(C(=O)N3CCN(c4ccc5c(c4)n(C)c(=O)n5C4CCC(=O)NC4=O)CC3)cc2)C1] |
| 138 | 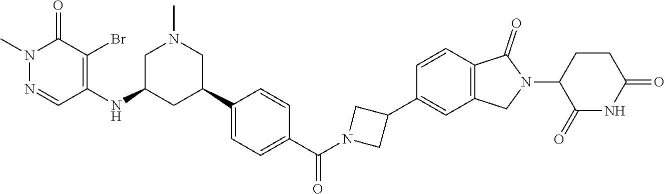 [SMILES: CN1C[C@H](Nc2cnn(C)c(=O)c2Br)C[C@H](c2ccc(C(=O)N3CC(c4ccc5c(c4)CN(C4CCC(=O)NC4=O)C5=O)C3)cc2)C1] |
| 138a | 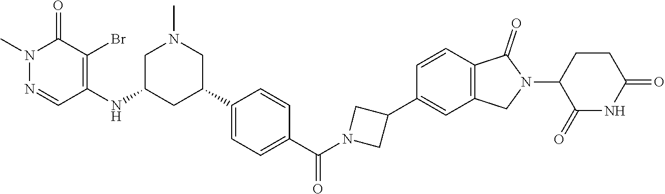 [SMILES: CN1C[C@@H](Nc2cnn(C)c(=O)c2Br)C[C@@H](c2ccc(C(=O)N3CC(c4ccc5c(c4)CN(C4CCC(=O)NC4=O)C5=O)C3)cc2)C1] |
| 138b | 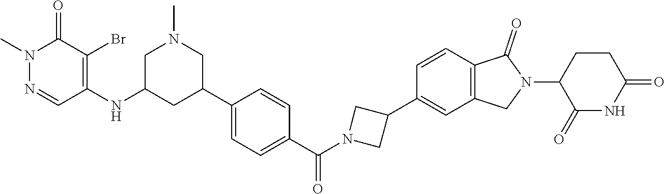 [SMILES: CN1CC(Nc2cnn(C)c(=O)c2Br)CC(c2ccc(C(=O)N3CC(c4ccc5c(c4)CN(C4CCC(=O)NC4=O)C5=O)C3)cc2)C1] |
| 139 | 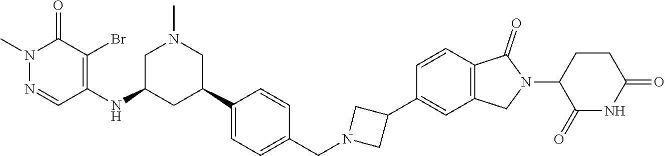 [SMILES: CN1C[C@H](Nc2cnn(C)c(=O)c2Br)C[C@H](c2ccc(CN3CC(c4ccc5c(c4)CN(C4CCC(=O)NC4=O)C5=O)C3)cc2)C1] |
| 139a | 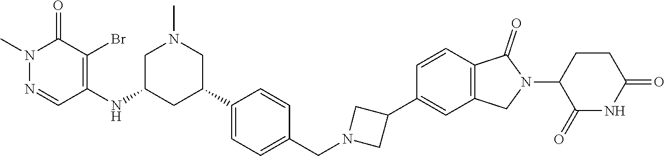 [SMILES: CN1C[C@@H](Nc2cnn(C)c(=O)c2Br)C[C@@H](c2ccc(CN3CC(c4ccc5c(c4)CN(C4CCC(=O)NC4=O)C5=O)C3)cc2)C1] |
| 139b | 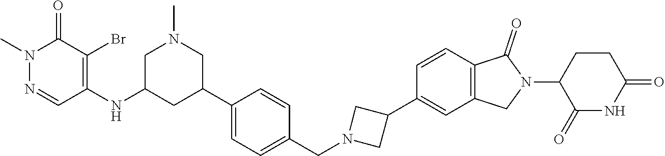 [SMILES: CN1CC(Nc2cnn(C)c(=O)c2Br)CC(c2ccc(CN3CC(c4ccc5c(c4)CN(C4CCC(=O)NC4=O)C5=O)C3)cc2)C1] |
| 140 | 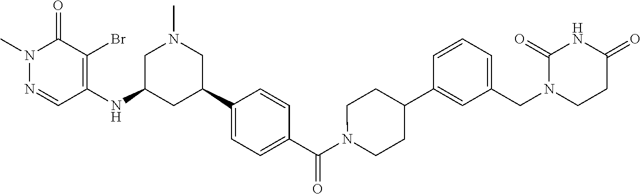 [SMILES: CN1C[C@H](Nc2cnn(C)c(=O)c2Br)C[C@H](c2ccc(C(=O)N3CCC(c4cccc(CN5CCC(=O)NC5=O)c4)CC3)cc2)C1] |
| 140a | 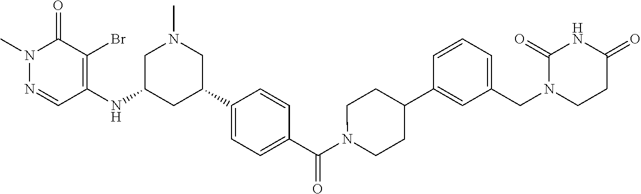 [SMILES: CN1C[C@@H](Nc2cnn(C)c(=O)c2Br)C[C@@H](c2ccc(C(=O)N3CCC(c4cccc(CN5CCC(=O)NC5=O)c4)CC3)cc2)C1] |
| 140b | 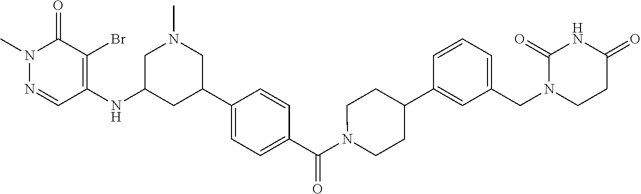 [SMILES: CN1CC(Nc2cnn(C)c(=O)c2Br)CC(c2ccc(C(=O)N3CCC(c4cccc(CN5CCC(=O)NC5=O)c4)CC3)cc2)C1] |
| 141 | 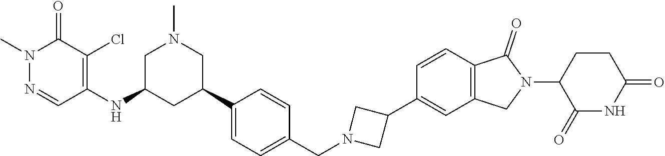 [SMILES: CN1C[C@H](Nc2cnn(C)c(=O)c2Cl)C[C@H](c2ccc(CN3CC(c4ccc5c(c4)CN(C4CCC(=O)NC4=O)C5=O)C3)cc2)C1] |
| 141a | 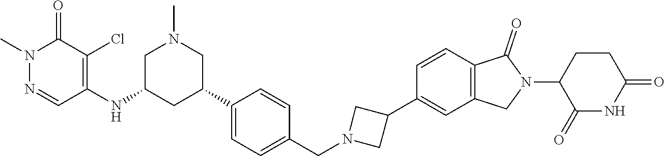 [SMILES: CN1C[C@@H](Nc2cnn(C)c(=O)c2Cl)C[C@@H](c2ccc(CN3CC(c4ccc5c(c4)CN(C4CCC(=O)NC4=O)C5=O)C3)cc2)C1] |
| 141b | 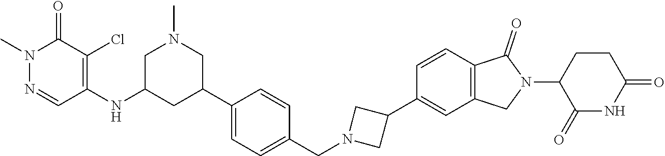 [SMILES: CN1CC(Nc2cnn(C)c(=O)c2Cl)CC(c2ccc(CN3CC(c4ccc5c(c4)CN(C4CCC(=O)NC4=O)C5=O)C3)cc2)C1] |
| 142 | 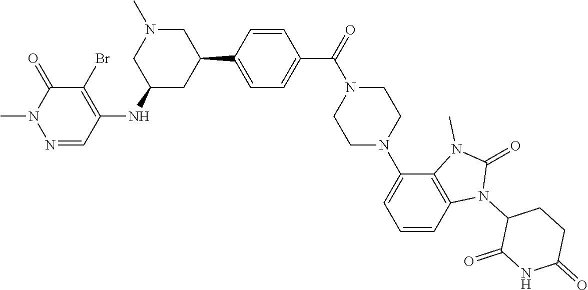 [SMILES: CN1C[C@H](Nc2cnn(C)c(=O)c2Br)C[C@H](c2ccc(C(=O)N3CCN(c4cccc5c4n(C)c(=O)n5C4CCC(=O)NC4=O)CC3)cc2)C1] |
| 142a | 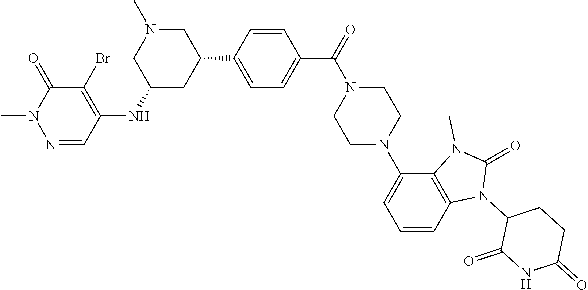 [SMILES: CN1C[C@@H](Nc2cnn(C)c(=O)c2Br)C[C@@H](c2ccc(C(=O)N3CCN(c4cccc5c4n(C)c(=O)n5C4CCC(=O)NC4=O)CC3)cc2)C1] |
| 142b | 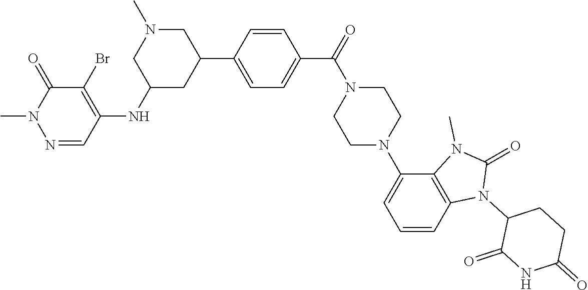 [SMILES: CN1CC(Nc2cnn(C)c(=O)c2Br)CC(c2ccc(C(=O)N3CCN(c4cccc5c4n(C)c(=O)n5C4CCC(=O)NC4=O)CC3)cc2)C1] |
| 143 | 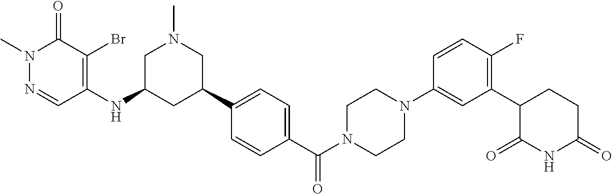 [SMILES: CN1C[C@H](Nc2cnn(C)c(=O)c2Br)C[C@H](c2ccc(C(=O)N3CCN(c4ccc(F)c(C5CCC(=O)NC5=O)c4)CC3)cc2)C1] |
| 143a | 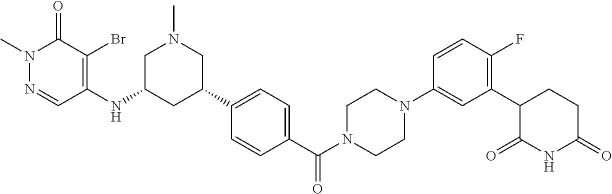 [SMILES: CN1C[C@@H](Nc2cnn(C)c(=O)c2Br)C[C@@H](c2ccc(C(=O)N3CCN(c4ccc(F)c(C5CCC(=O)NC5=O)c4)CC3)cc2)C1] |
| 143b | 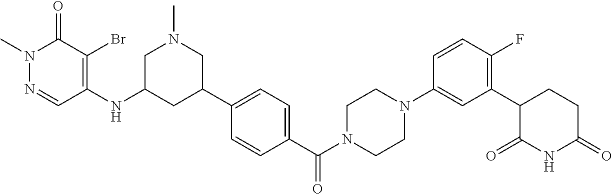 [SMILES: CN1CC(Nc2cnn(C)c(=O)c2Br)CC(c2ccc(C(=O)N3CCN(c4ccc(F)c(C5CCC(=O)NC5=O)c4)CC3)cc2)C1] |
| 144 | 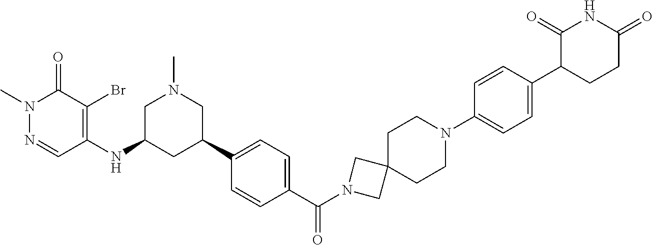 [SMILES: CN1C[C@H](Nc2cnn(C)c(=O)c2Br)C[C@H](c2ccc(C(=O)N3CC4(CCN(c5ccc(C6CCC(=O)NC6=O)cc5)CC4)C3)cc2)C1] |
| 144a | 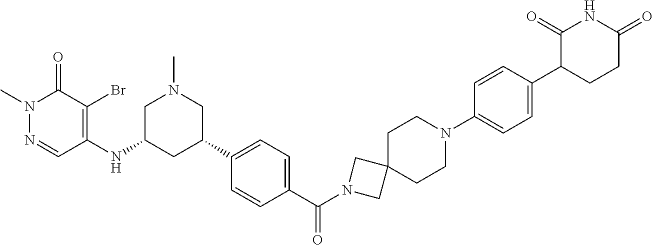 [SMILES: CN1C[C@@H](Nc2cnn(C)c(=O)c2Br)C[C@@H](c2ccc(C(=O)N3CC4(CCN(c5ccc(C6CCC(=O)NC6=O)cc5)CC4)C3)cc2)C1] |
| 144b | 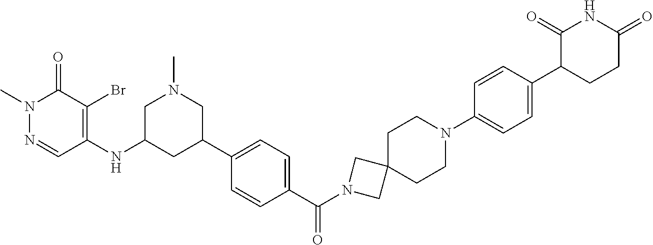 [SMILES: CN1CC(Nc2cnn(C)c(=O)c2Br)CC(c2ccc(C(=O)N3CC4(CCN(c5ccc(C6CCC(=O)NC6=O)cc5)CC4)C3)cc2)C1] |
| 145 | 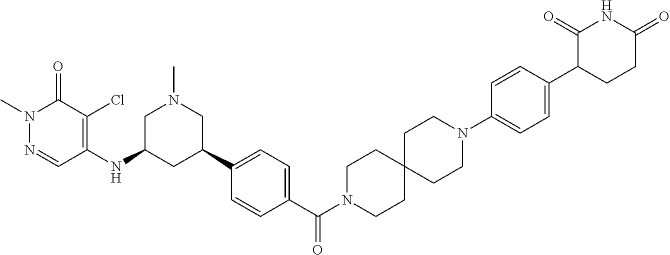 [SMILES: CN1C[C@H](Nc2cnn(C)c(=O)c2Cl)C[C@H](c2ccc(C(=O)N3CCC4(CC3)CCN(c3ccc(C5CCC(=O)NC5=O)cc3)CC4)cc2)C1] |
| 145-1 | 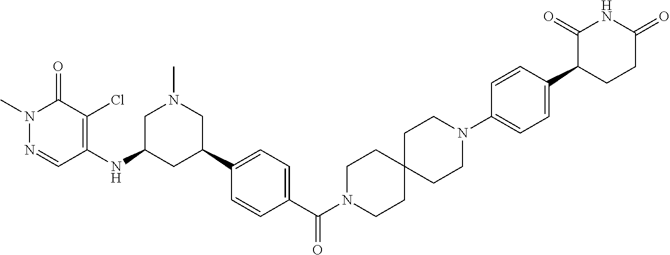 [SMILES: CN1C[C@H](Nc2cnn(C)c(=O)c2Cl)C[C@H](c2ccc(C(=O)N3CCC4(CC3)CCN(c3ccc([C@@H]5CCC(=O)NC5=O)cc3)CC4)cc2)C1] |
| 145-2 | 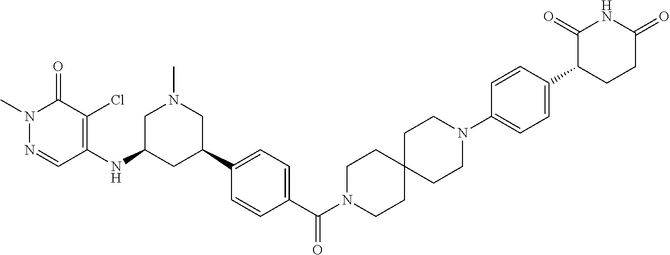 [SMILES: CN1C[C@H](Nc2cnn(C)c(=O)c2Cl)C[C@H](c2ccc(C(=O)N3CCC4(CC3)CCN(c3ccc([C@H]5CCC(=O)NC5=O)cc3)CC4)cc2)C1] |
| 145a | 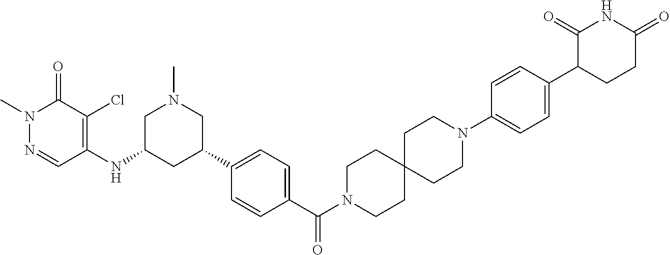 [SMILES: CN1C[C@@H](Nc2cnn(C)c(=O)c2Cl)C[C@@H](c2ccc(C(=O)N3CCC4(CC3)CCN(c3ccc(C5CCC(=O)NC5=O)cc3)CC4)cc2)C1] |
| 145b | 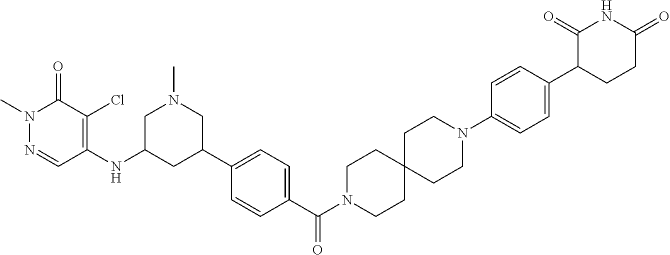 [SMILES: CN1CC(Nc2cnn(C)c(=O)c2Cl)CC(c2ccc(C(=O)N3CCC4(CC3)CCN(c3ccc(C5CCC(=O)NC5=O)cc3)CC4)cc2)C1] |
| 146 | 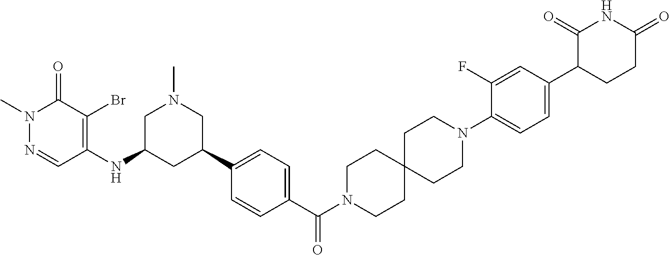 [SMILES: CN1C[C@H](Nc2cnn(C)c(=O)c2Br)C[C@H](c2ccc(C(=O)N3CCC4(CC3)CCN(c3ccc(C5CCC(=O)NC5=O)cc3F)CC4)cc2)C1] |
| 146a | 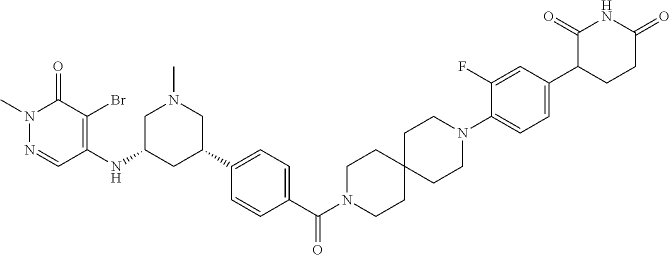 [SMILES: CN1C[C@@H](Nc2cnn(C)c(=O)c2Br)C[C@@H](c2ccc(C(=O)N3CCC4(CC3)CCN(c3ccc(C5CCC(=O)NC5=O)cc3F)CC4)cc2)C1] |
| 146b | 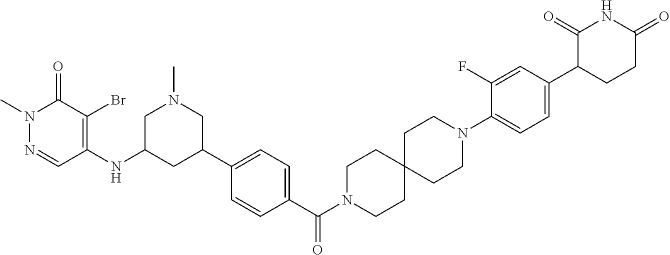 [SMILES: CN1CC(Nc2cnn(C)c(=O)c2Br)CC(c2ccc(C(=O)N3CCC4(CC3)CCN(c3ccc(C5CCC(=O)NC5=O)cc3F)CC4)cc2)C1] |
| 147 | 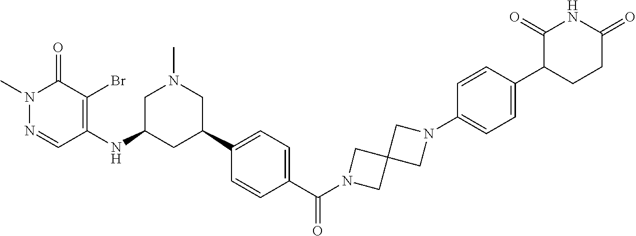 [SMILES: CN1C[C@H](Nc2cnn(C)c(=O)c2Br)C[C@H](c2ccc(C(=O)N3CC4(C3)CN(c3ccc(C5CCC(=O)NC5=O)cc3)C4)cc2)C1] |
| 147a | 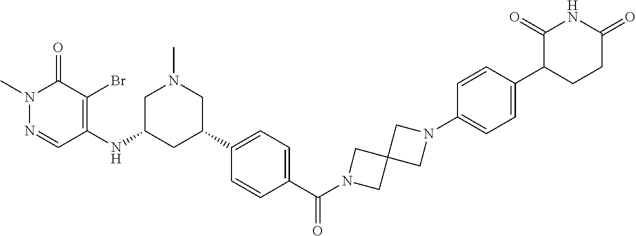 [SMILES: CN1C[C@@H](Nc2cnn(C)c(=O)c2Br)C[C@@H](c2ccc(C(=O)N3CC4(C3)CN(c3ccc(C5CCC(=O)NC5=O)cc3)C4)cc2)C1] |
| 147b | 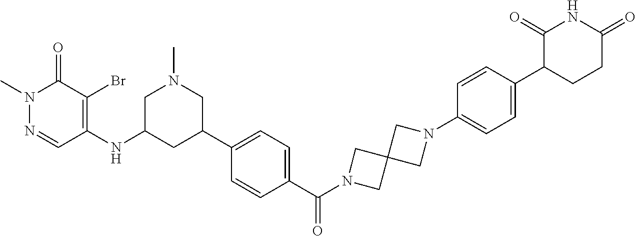 [SMILES: CN1CC(Nc2cnn(C)c(=O)c2Br)CC(c2ccc(C(=O)N3CC4(C3)CN(c3ccc(C5CCC(=O)NC5=O)cc3)C4)cc2)C1] |
| 148 | 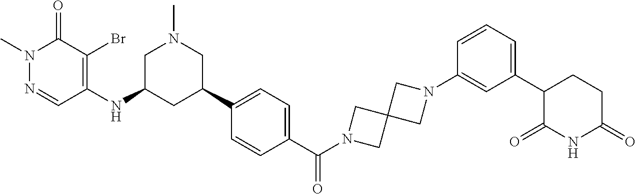 [SMILES: CN1C[C@H](Nc2cnn(C)c(=O)c2Br)C[C@H](c2ccc(C(=O)N3CC4(C3)CN(c3cccc(C5CCC(=O)NC5=O)c3)C4)cc2)C1] |
| 148a | 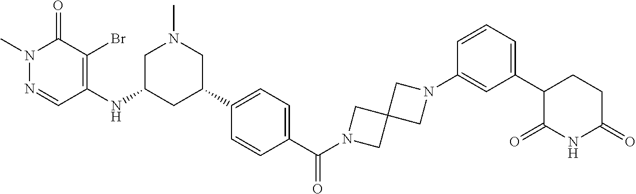 [SMILES: CN1C[C@@H](Nc2cnn(C)c(=O)c2Br)C[C@@H](c2ccc(C(=O)N3CC4(C3)CN(c3cccc(C5CCC(=O)NC5=O)c3)C4)cc2)C1] |
| 148b | 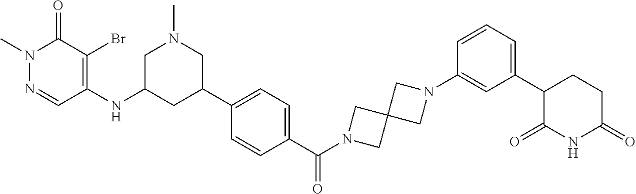 [SMILES: CN1CC(Nc2cnn(C)c(=O)c2Br)CC(c2ccc(C(=O)N3CC4(C3)CN(c3cccc(C5CCC(=O)NC5=O)c3)C4)cc2)C1] |
| 149 | 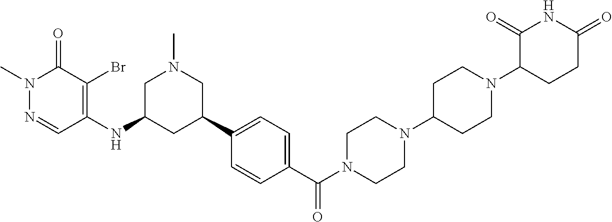 [SMILES: CN1C[C@H](Nc2cnn(C)c(=O)c2Br)C[C@H](c2ccc(C(=O)N3CCN(C4CCN(C5CCC(=O)NC5=O)CC4)CC3)cc2)C1] |
| 149a | 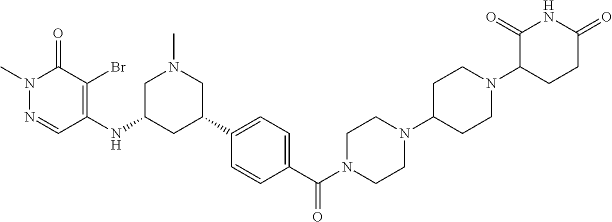 [SMILES: CN1C[C@@H](Nc2cnn(C)c(=O)c2Br)C[C@@H](c2ccc(C(=O)N3CCN(C4CCN(C5CCC(=O)NC5=O)CC4)CC3)cc2)C1] |
| 149b | 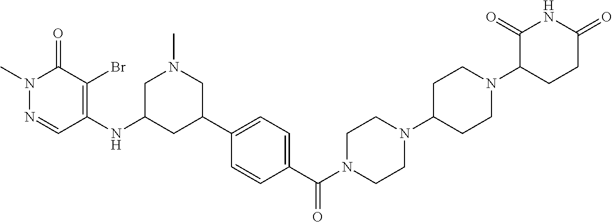 [SMILES: CN1CC(Nc2cnn(C)c(=O)c2Br)CC(c2ccc(C(=O)N3CCN(C4CCN(C5CCC(=O)NC5=O)CC4)CC3)cc2)C1] |
| 150 | 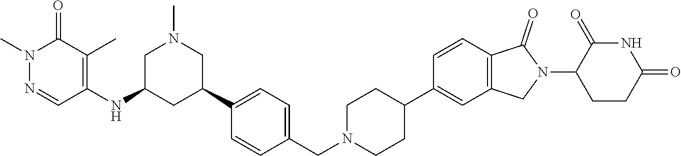 [SMILES: Cc1c(N[C@@H]2C[C@H](c3ccc(CN4CCC(c5ccc6c(c5)CN(C5CCC(=O)NC5=O)C6=O)CC4)cc3)CN(C)C2)cnn(C)c1=O] |
| 150a | 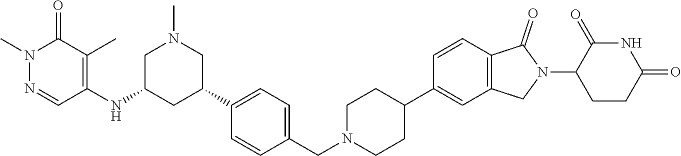 [SMILES: Cc1c(N[C@H]2C[C@@H](c3ccc(CN4CCC(c5ccc6c(c5)CN(C5CCC(=O)NC5=O)C6=O)CC4)cc3)CN(C)C2)cnn(C)c1=O] |
| 150b | 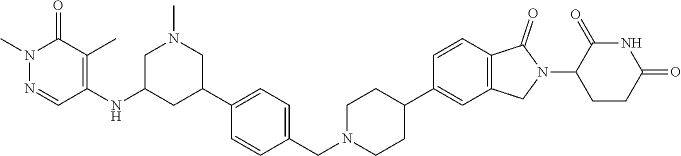 [SMILES: Cc1c(NC2CC(c3ccc(CN4CCC(c5ccc6c(c5)CN(C5CCC(=O)NC5=O)C6=O)CC4)cc3)CN(C)C2)cnn(C)c1=O] |
| 151 | 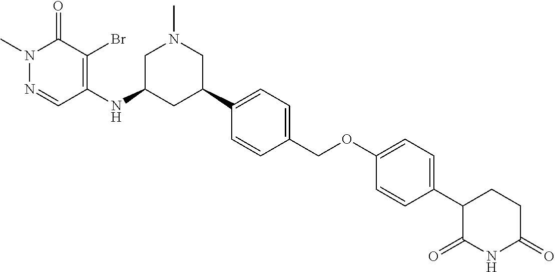 [SMILES: CN1C[C@H](Nc2cnn(C)c(=O)c2Br)C[C@H](c2ccc(COc3ccc(C4CCC(=O)NC4=O)cc3)cc2)C1] |
| 151a | 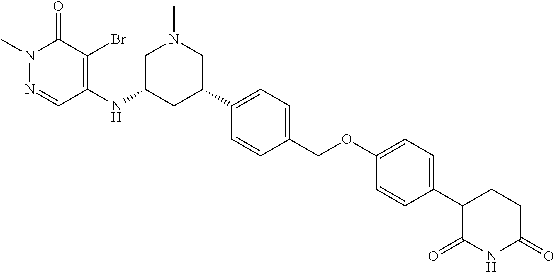 [SMILES: CN1C[C@@H](Nc2cnn(C)c(=O)c2Br)C[C@@H](c2ccc(COc3ccc(C4CCC(=O)NC4=O)cc3)cc2)C1] |
| 151b | 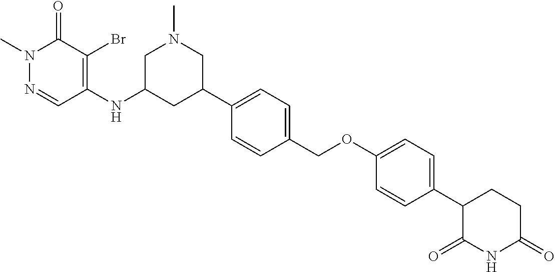 [SMILES: CN1CC(Nc2cnn(C)c(=O)c2Br)CC(c2ccc(COc3ccc(C4CCC(=O)NC4=O)cc3)cc2)C1] |
| 152 | 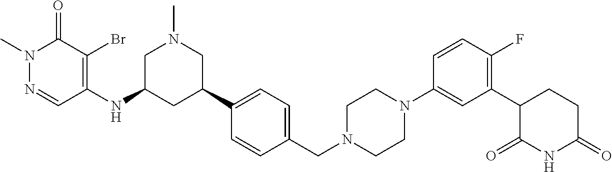 [SMILES: CN1C[C@H](Nc2cnn(C)c(=O)c2Br)C[C@H](c2ccc(CN3CCN(c4ccc(F)c(C5CCC(=O)NC5=O)c4)CC3)cc2)C1] |
| 152a | 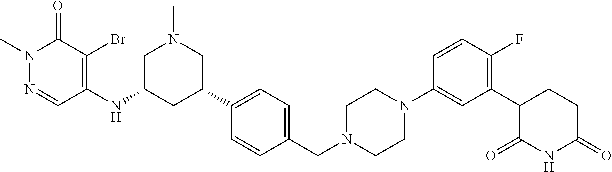 [SMILES: CN1C[C@@H](Nc2cnn(C)c(=O)c2Br)C[C@@H](c2ccc(CN3CCN(c4ccc(F)c(C5CCC(=O)NC5=O)c4)CC3)cc2)C1] |
| 152b | 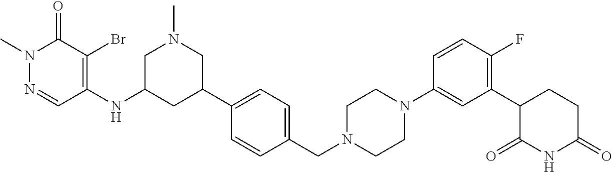 [SMILES: CN1CC(Nc2cnn(C)c(=O)c2Br)CC(c2ccc(CN3CCN(c4ccc(F)c(C5CCC(=O)NC5=O)c4)CC3)cc2)C1] |
| 153 | 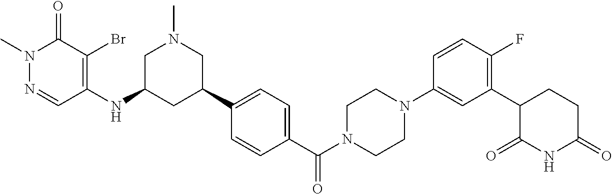 [SMILES: CN1C[C@H](Nc2cnn(C)c(=O)c2Br)C[C@H](c2ccc(C(=O)N3CCN(c4ccc(F)c(C5CCC(=O)NC5=O)c4)CC3)cc2)C1] |
| 153a | 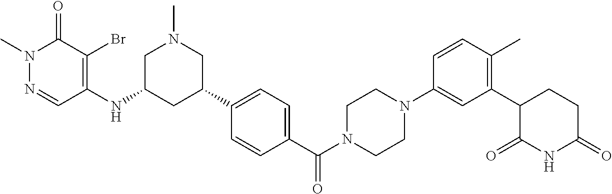 [SMILES: Cc1ccc(N2CCN(C(=O)c3ccc([C@@H]4C[C@H](Nc5cnn(C)c(=O)c5Br)CN(C)C4)cc3)CC2)cc1C1CCC(=O)NC1=O] |
| 153b | 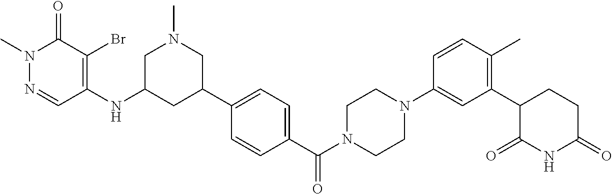 [SMILES: Cc1ccc(N2CCN(C(=O)c3ccc(C4CC(Nc5cnn(C)c(=O)c5Br)CN(C)C4)cc3)CC2)cc1C1CCC(=O)NC1=O] |
| 154 | 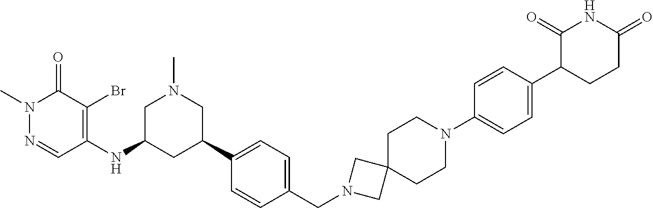 [SMILES: CN1C[C@H](Nc2cnn(C)c(=O)c2Br)C[C@H](c2ccc(CN3CC4(CCN(c5ccc(C6CCC(=O)NC6=O)cc5)CC4)C3)cc2)C1] |
| 154a | 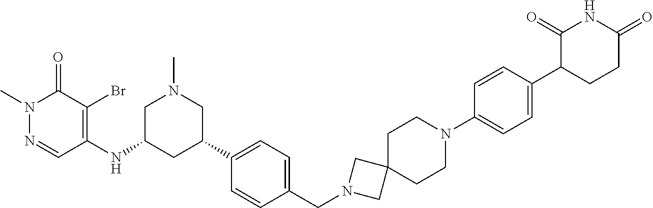 [SMILES: CN1C[C@@H](Nc2cnn(C)c(=O)c2Br)C[C@@H](c2ccc(CN3CC4(CCN(c5ccc(C6CCC(=O)NC6=O)cc5)CC4)C3)cc2)C1] |
| 154b | 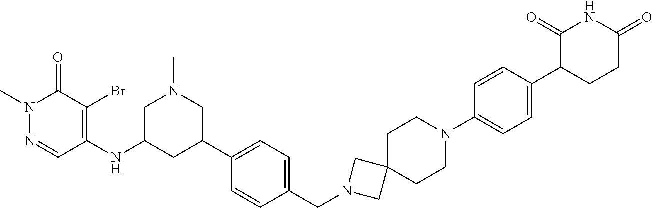 [SMILES: CN1CC(Nc2cnn(C)c(=O)c2Br)CC(c2ccc(CN3CC4(CCN(c5ccc(C6CCC(=O)NC6=O)cc5)CC4)C3)cc2)C1] |
| 155 | 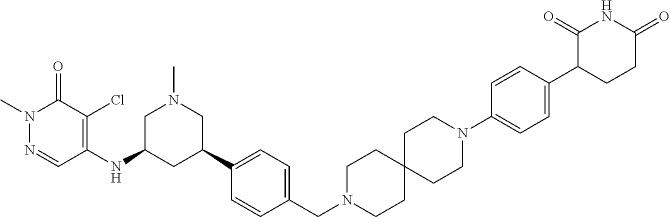 [SMILES: CN1C[C@H](Nc2cnn(C)c(=O)c2Cl)C[C@H](c2ccc(CN3CCC4(CC3)CCN(c3ccc(C5CCC(=O)NC5=O)cc3)CC4)cc2)C1] |
| 155a | 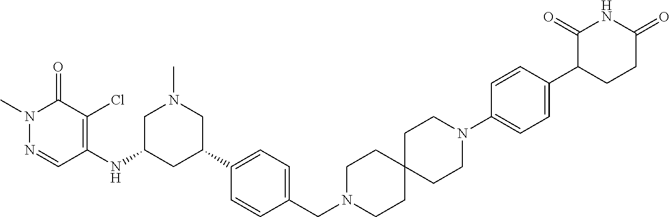 [SMILES: CN1C[C@@H](Nc2cnn(C)c(=O)c2Cl)C[C@@H](c2ccc(CN3CCC4(CC3)CCN(c3ccc(C5CCC(=O)NC5=O)cc3)CC4)cc2)C1] |
| 155b | 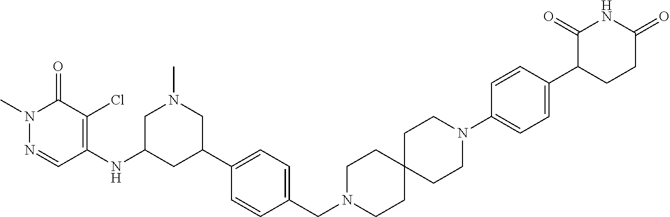 [SMILES: CN1CC(Nc2cnn(C)c(=O)c2Cl)CC(c2ccc(CN3CCC4(CC3)CCN(c3ccc(C5CCC(=O)NC5=O)cc3)CC4)cc2)C1] |
| 156 | 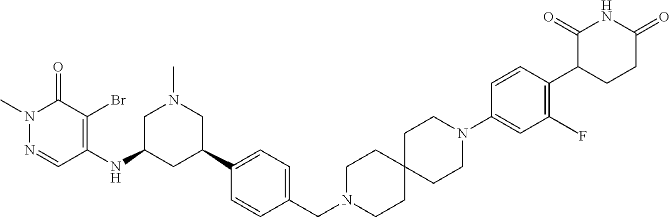 |
| 156a | 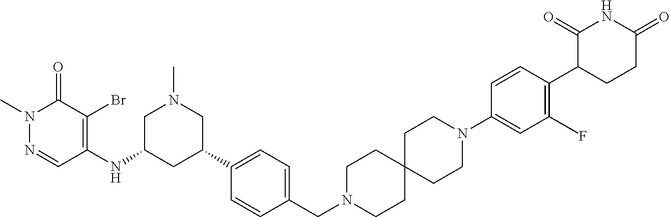 |
| 156b | 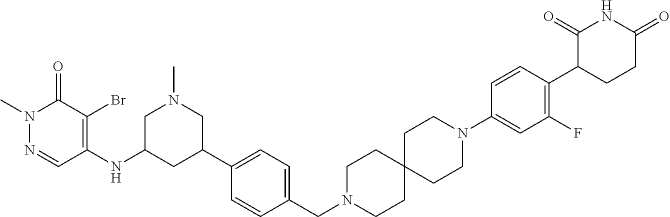 [SMILES: CN1CC(Nc2cnn(C)c(=O)c2Br)CC(c2ccc(CN3CCC4(CC3)CCN(c3ccc(C5CCC(=O)NC5=O)c(F)c3)CC4)cc2)C1] |
| 157 | 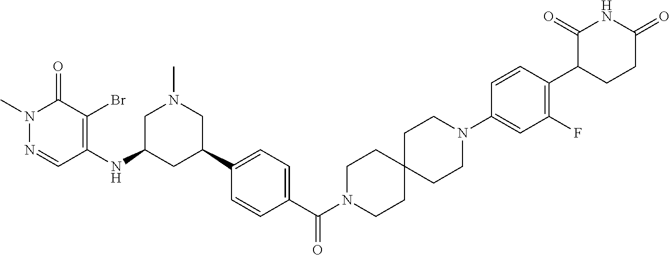 [SMILES: CN1C[C@H](Nc2cnn(C)c(=O)c2Br)C[C@H](c2ccc(C(=O)N3CCC4(CC3)CCN(c3ccc(C5CCC(=O)NC5=O)c(F)c3)CC4)cc2)C1] |
| 157-1 | 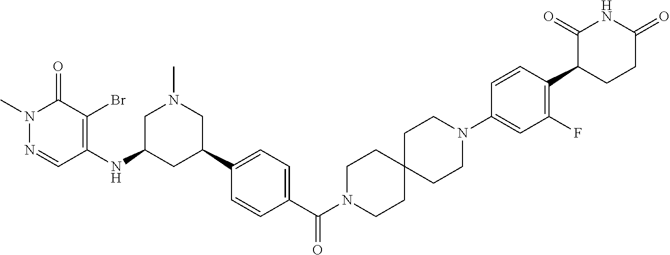 [SMILES: CN1C[C@H](Nc2cnn(C)c(=O)c2Br)C[C@H](c2ccc(C(=O)N3CCC4(CC3)CCN(c3ccc([C@@H]5CCC(=O)NC5=O)c(F)c3)CC4)cc2)C1] |
| 157-2 | 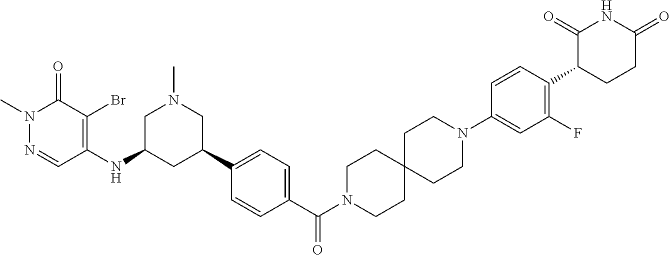 [SMILES: CN1C[C@H](Nc2cnn(C)c(=O)c2Br)C[C@H](c2ccc(C(=O)N3CCC4(CC3)CCN(c3ccc([C@H]5CCC(=O)NC5=O)c(F)c3)CC4)cc2)C1] |
| 157a | 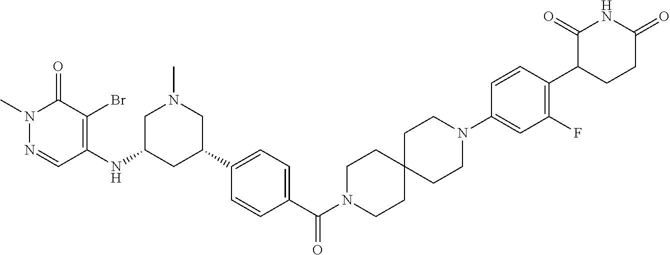 [SMILES: CN1C[C@@H](Nc2cnn(C)c(=O)c2Br)C[C@@H](c2ccc(C(=O)N3CCC4(CC3)CCN(c3ccc(C5CCC(=O)NC5=O)c(F)c3)CC4)cc2)C1] |
| 157b | 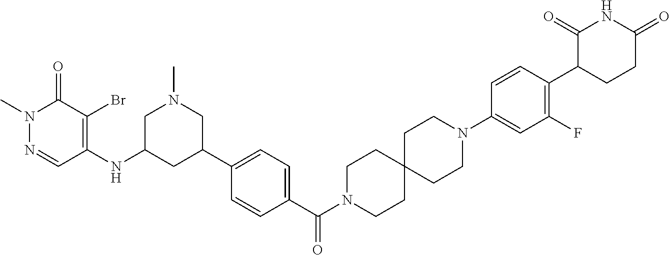 [SMILES: CN1CC(Nc2cnn(C)c(=O)c2Br)CC(c2ccc(C(=O)N3CCC4(CC3)CCN(c3ccc(C5CCC(=O)NC5=O)c(F)c3)CC4)cc2)C1] |
| 158 | 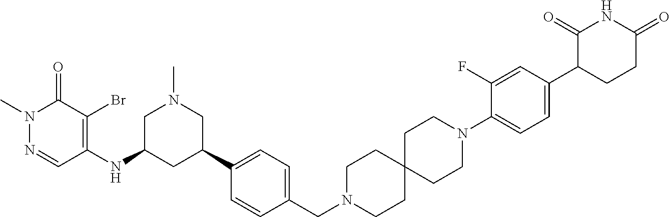 [SMILES: CN1C[C@H](Nc2cnn(C)c(=O)c2Br)C[C@H](c2ccc(CN3CCC4(CC3)CCN(c3ccc(C5CCC(=O)NC5=O)cc3F)CC4)cc2)C1] |
| 158a | 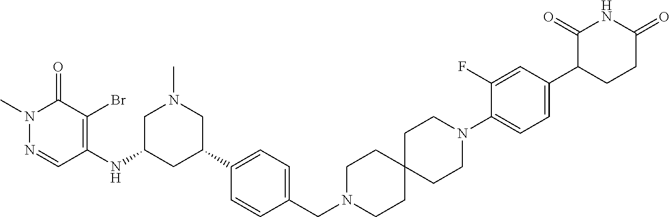 [SMILES: CN1C[C@@H](Nc2cnn(C)c(=O)c2Br)C[C@@H](c2ccc(CN3CCC4(CC3)CCN(c3ccc(C5CCC(=O)NC5=O)cc3F)CC4)cc2)C1] |
| 158b | 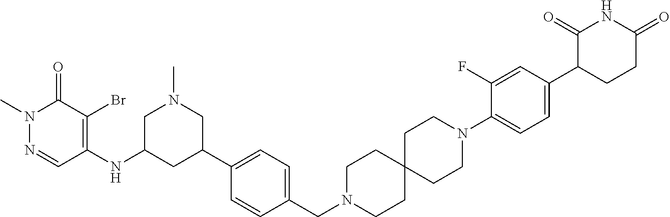 [SMILES: CN1CC(Nc2cnn(C)c(=O)c2Br)CC(c2ccc(CN3CCC4(CC3)CCN(c3ccc(C5CCC(=O)NC5=O)cc3F)CC4)cc2)C1] |
| 159 | 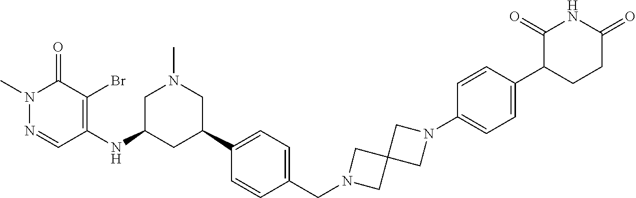 [SMILES: CN1C[C@H](Nc2cnn(C)c(=O)c2Br)C[C@H](c2ccc(CN3CC4(C3)CN(c3ccc(C5CCC(=O)NC5=O)cc3)C4)cc2)C1] |
| 159a | 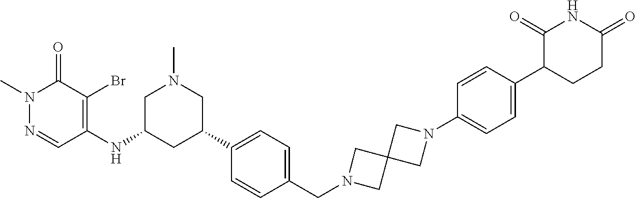 [SMILES: CN1C[C@@H](Nc2cnn(C)c(=O)c2Br)C[C@@H](c2ccc(CN3CC4(C3)CN(c3ccc(C5CCC(=O)NC5=O)cc3)C4)cc2)C1] |
| 159b | 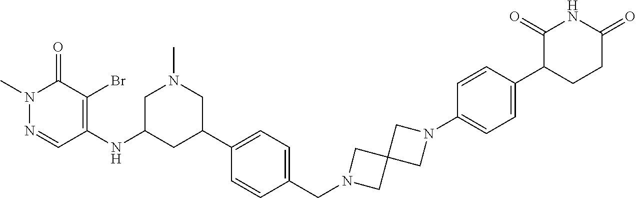 [SMILES: CN1CC(Nc2cnn(C)c(=O)c2Br)CC(c2ccc(CN3CC4(C3)CN(c3ccc(C5CCC(=O)NC5=O)cc3)C4)cc2)C1] |
| 160 | 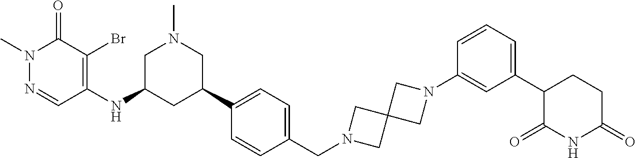 [SMILES: CN1C[C@H](Nc2cnn(C)c(=O)c2Br)C[C@H](c2ccc(CN3CC4(C3)CN(c3cccc(C5CCC(=O)NC5=O)c3)C4)cc2)C1] |
| 160a | 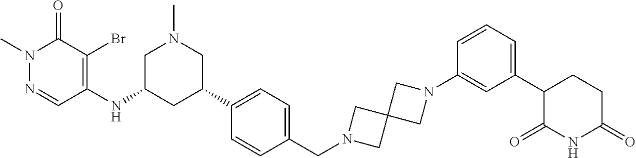 [SMILES: CN1C[C@@H](Nc2cnn(C)c(=O)c2Br)C[C@@H](c2ccc(CN3CC4(C3)CN(c3cccc(C5CCC(=O)NC5=O)c3)C4)cc2)C1] |
| 160b | 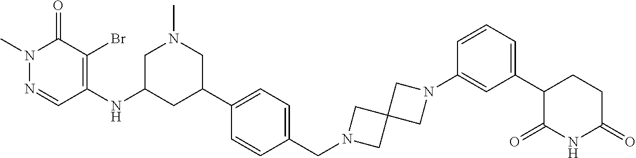 [SMILES: CN1CC(Nc2cnn(C)c(=O)c2Br)CC(c2ccc(CN3CC4(C3)CN(c3cccc(C5CCC(=O)NC5=O)c3)C4)cc2)C1] |
| 161 | 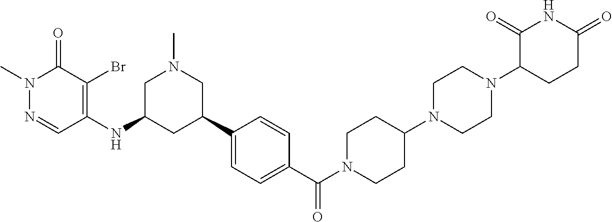 [SMILES: CN1C[C@H](Nc2cnn(C)c(=O)c2Br)C[C@H](c2ccc(C(=O)N3CCC(N4CCN(C5CCC(=O)NC5=O)CC4)CC3)cc2)C1] |
| 161a | 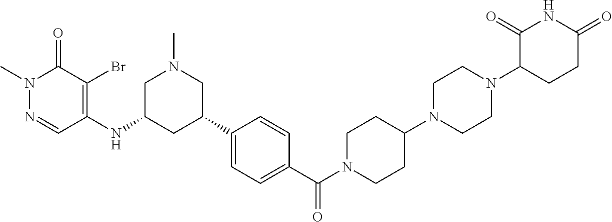 [SMILES: CN1C[C@@H](Nc2cnn(C)c(=O)c2Br)C[C@@H](c2ccc(C(=O)N3CCC(N4CCN(C5CCC(=O)NC5=O)CC4)CC3)cc2)C1] |
| 161b | 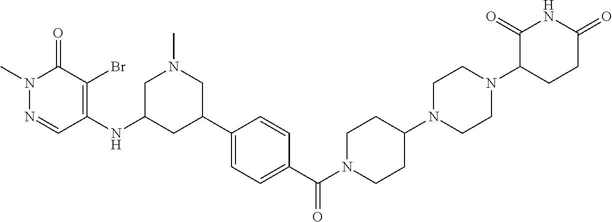 [SMILES: CN1CC(Nc2cnn(C)c(=O)c2Br)CC(c2ccc(C(=O)N3CCC(N4CCN(C5CCC(=O)NC5=O)CC4)CC3)cc2)C1] |
| 162 | 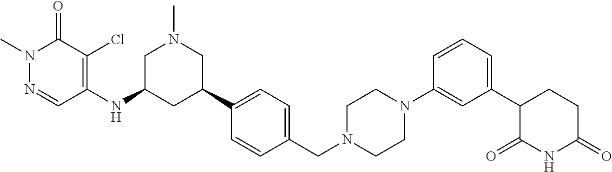 [SMILES: CN1C[C@H](Nc2cnn(C)c(=O)c2Cl)C[C@H](c2ccc(CN3CCN(c4cccc(C5CCC(=O)NC5=O)c4)CC3)cc2)C1] |
| 162a | 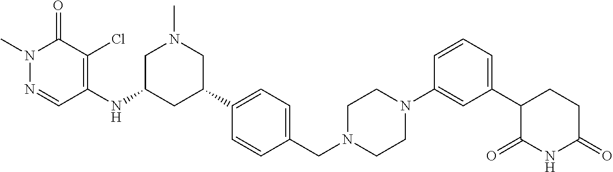 [SMILES: CN1C[C@@H](Nc2cnn(C)c(=O)c2Cl)C[C@@H](c2ccc(CN3CCN(c4cccc(C5CCC(=O)NC5=O)c4)CC3)cc2)C1] |
| 162b | 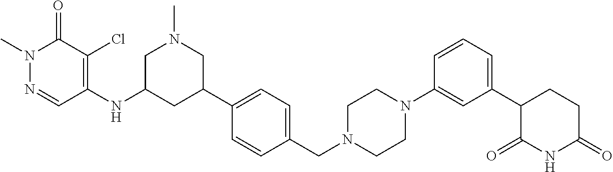 [SMILES: CN1CC(Nc2cnn(C)c(=O)c2Cl)CC(c2ccc(CN3CCN(c4cccc(C5CCC(=O)NC5=O)c4)CC3)cc2)C1] |
| 163 | 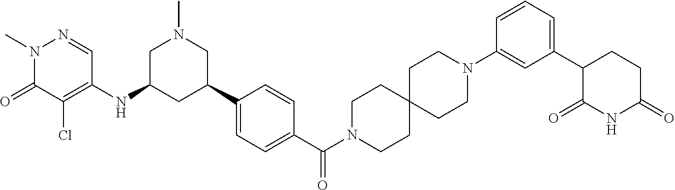 [SMILES: CN1C[C@H](Nc2cnn(C)c(=O)c2Cl)C[C@H](c2ccc(C(=O)N3CCC4(CC3)CCN(c3cccc(C5CCC(=O)NC5=O)c3)CC4)cc2)C1] |
| 163a | 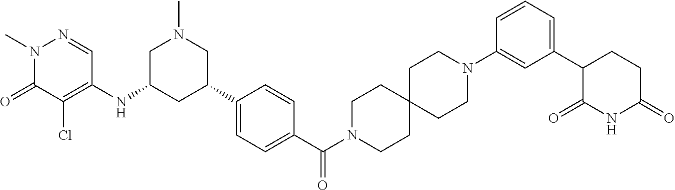 [SMILES: CN1C[C@@H](Nc2cnn(C)c(=O)c2Cl)C[C@@H](c2ccc(C(=O)N3CCC4(CC3)CCN(c3cccc(C5CCC(=O)NC5=O)c3)CC4)cc2)C1] |
| 163b | 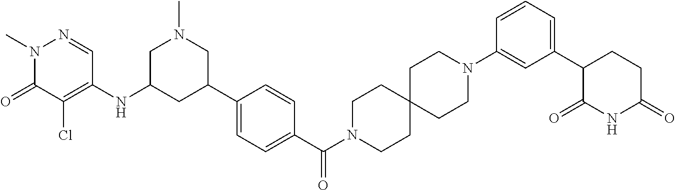 [SMILES: CN1CC(Nc2cnn(C)c(=O)c2Cl)CC(c2ccc(C(=O)N3CCC4(CC3)CCN(c3cccc(C5CCC(=O)NC5=O)c3)CC4)cc2)C1] |
| 164 | 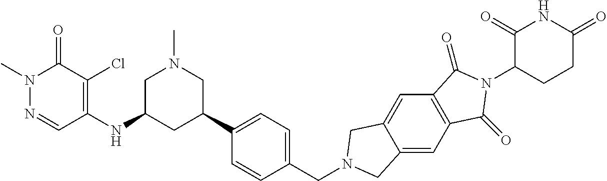 [SMILES: CN1C[C@H](Nc2cnn(C)c(=O)c2Cl)C[C@H](c2ccc(CN3Cc4cc5c(cc4C3)C(=O)N(C3CCC(=O)NC3=O)C5=O)cc2)C1] |
| 164a | 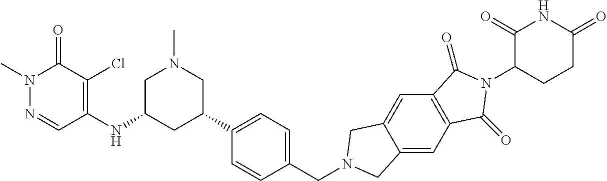 [SMILES: CN1C[C@@H](Nc2cnn(C)c(=O)c2Cl)C[C@@H](c2ccc(CN3Cc4cc5c(cc4C3)C(=O)N(C3CCC(=O)NC3=O)C5=O)cc2)C1] |
| 164b | 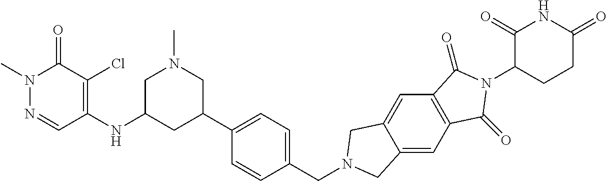 [SMILES: CN1CC(Nc2cnn(C)c(=O)c2Cl)CC(c2ccc(CN3Cc4cc5c(cc4C3)C(=O)N(C3CCC(=O)NC3=O)C5=O)cc2)C1] |
| 165 | 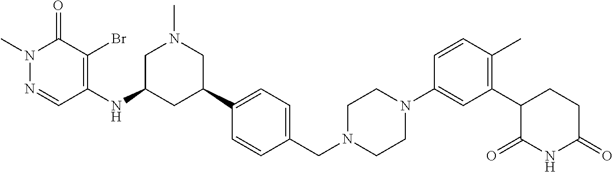 [SMILES: Cc1ccc(N2CCN(Cc3ccc([C@H]4C[C@@H](Nc5cnn(C)c(=O)c5Br)CN(C)C4)cc3)CC2)cc1C1CCC(=O)NC1=O] |
| 165a | 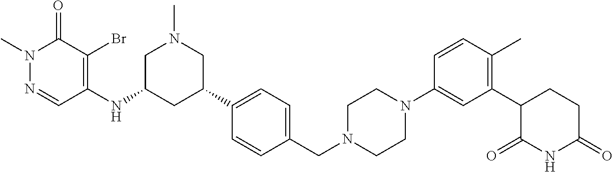 [SMILES: Cc1ccc(N2CCN(Cc3ccc([C@@H]4C[C@H](Nc5cnn(C)c(=O)c5Br)CN(C)C4)cc3)CC2)cc1C1CCC(=O)NC1=O] |
| 165b | 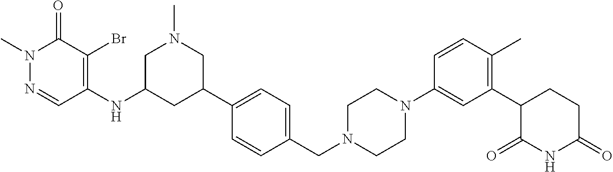 [SMILES: Cc1ccc(N2CCN(Cc3ccc(C4CC(Nc5cnn(C)c(=O)c5Br)CN(C)C4)cc3)CC2)cc1C1CCC(=O)NC1=O] |
| 166 | 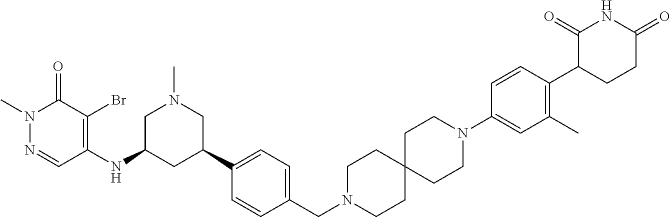 [SMILES: Cc1cc(N2CCC3(CCN(Cc4ccc([C@H]5C[C@@H](Nc6cnn(C)c(=O)c6Br)CN(C)C5)cc4)CC3)CC2)ccc1C1CCC(=O)NC1=O] |
| 166a | 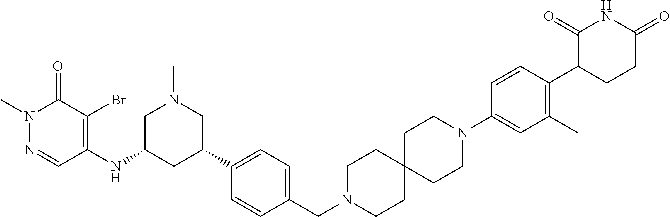 [SMILES: Cc1cc(N2CCC3(CCN(Cc4ccc([C@@H]5C[C@H](Nc6cnn(C)c(=O)c6Br)CN(C)C5)cc4)CC3)CC2)ccc1C1CCC(=O)NC1=O] |
| 166b | 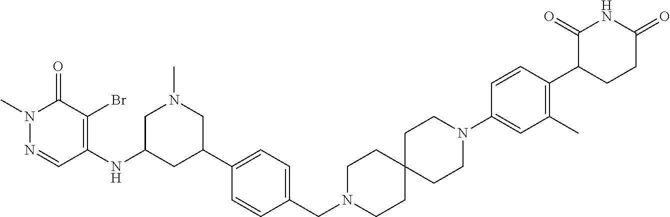 [SMILES: Cc1cc(N2CCC3(CCN(Cc4ccc(C5CC(Nc6cnn(C)c(=O)c6Br)CN(C)C5)cc4)CC3)CC2)ccc1C1CCC(=O)NC1=O] |
| 167 | 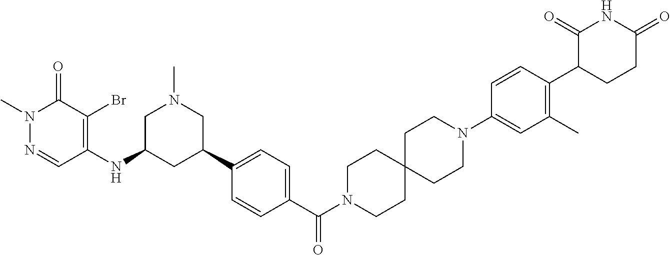 [SMILES: Cc1cc(N2CCC3(CCN(C(=O)c4ccc([C@H]5C[C@@H](Nc6cnn(C)c(=O)c6Br)CN(C)C5)cc4)CC3)CC2)ccc1C1CCC(=O)NC1=O] |
| 167a | 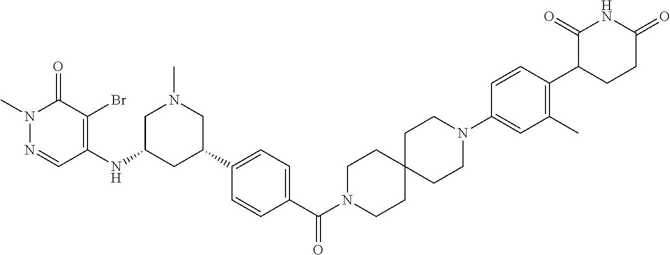 [SMILES: Cc1cc(N2CCC3(CCN(C(=O)c4ccc([C@@H]5C[C@H](Nc6cnn(C)c(=O)c6Br)CN(C)C5)cc4)CC3)CC2)ccc1C1CCC(=O)NC1=O] |
| 167b | 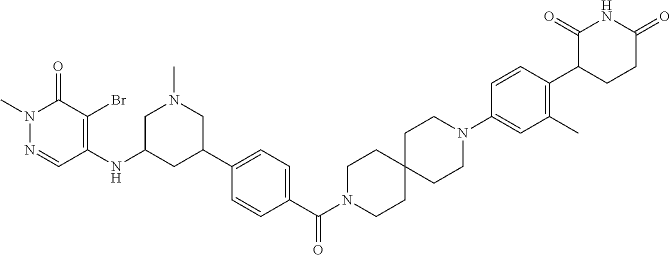 [SMILES: Cc1cc(N2CCC3(CCN(C(=O)c4ccc(C5CC(Nc6cnn(C)c(=O)c6Br)CN(C)C5)cc4)CC3)CC2)ccc1C1CCC(=O)NC1=O] |
| 168 | 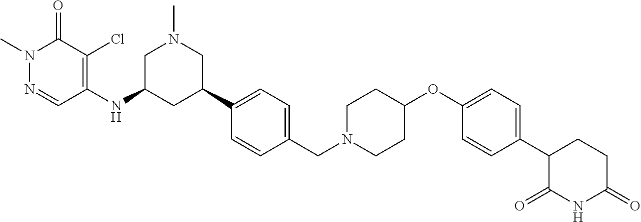 [SMILES: CN1C[C@H](Nc2cnn(C)c(=O)c2Cl)C[C@H](c2ccc(CN3CCC(Oc4ccc(C5CCC(=O)NC5=O)cc4)CC3)cc2)C1] |
| 168a | 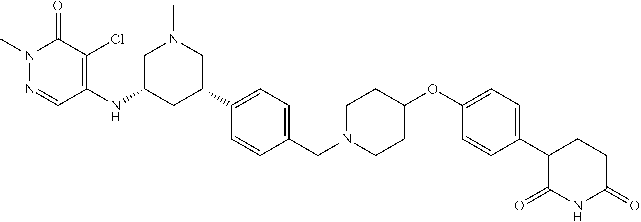 [SMILES: CN1C[C@@H](Nc2cnn(C)c(=O)c2Cl)C[C@@H](c2ccc(CN3CCC(Oc4ccc(C5CCC(=O)NC5=O)cc4)CC3)cc2)C1] |
| 168b | 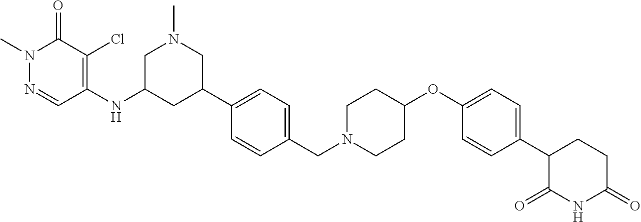 [SMILES: CN1CC(Nc2cnn(C)c(=O)c2Cl)CC(c2ccc(CN3CCC(Oc4ccc(C5CCC(=O)NC5=O)cc4)CC3)cc2)C1] |
| 169 | 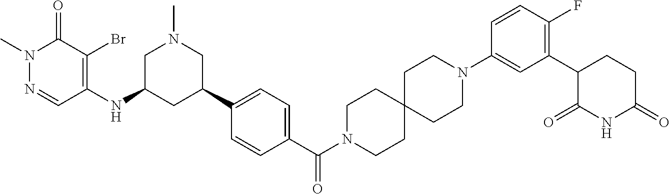 [SMILES: CN1C[C@H](Nc2cnn(C)c(=O)c2Br)C[C@H](c2ccc(C(=O)N3CCC4(CC3)CCN(c3ccc(F)c(C5CCC(=O)NC5=O)c3)CC4)cc2)C1] |
| 169a | 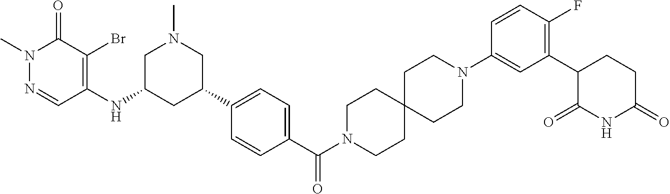 [SMILES: CN1C[C@@H](Nc2cnn(C)c(=O)c2Br)C[C@@H](c2ccc(C(=O)N3CCC4(CC3)CCN(c3ccc(F)c(C5CCC(=O)NC5=O)c3)CC4)cc2)C1] |
| 169b | 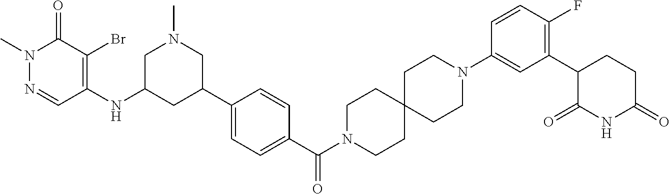 [SMILES: CN1CC(Nc2cnn(C)c(=O)c2Br)CC(c2ccc(C(=O)N3CCC4(CC3)CCN(c3ccc(F)c(C5CCC(=O)NC5=O)c3)CC4)cc2)C1] |
| 170 | 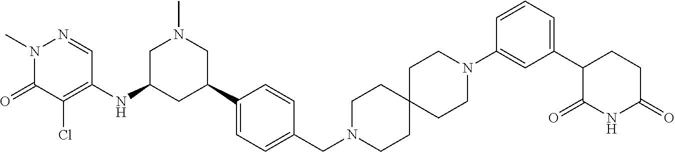 [SMILES: CN1C[C@H](Nc2cnn(C)c(=O)c2Cl)C[C@H](c2ccc(CN3CCC4(CC3)CCN(c3cccc(C5CCC(=O)NC5=O)c3)CC4)cc2)C1] |
| 170a | 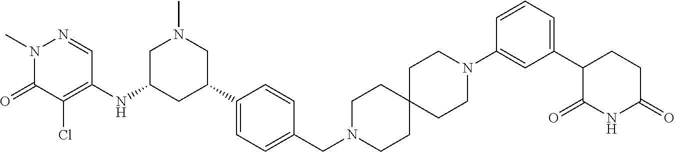 [SMILES: CN1C[C@@H](Nc2cnn(C)c(=O)c2Cl)C[C@@H](c2ccc(CN3CCC4(CC3)CCN(c3cccc(C5CCC(=O)NC5=O)c3)CC4)cc2)C1] |
| 170b | 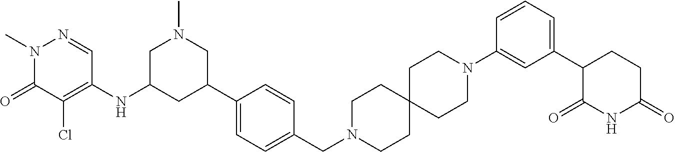 [SMILES: CN1CC(Nc2cnn(C)c(=O)c2Cl)CC(c2ccc(CN3CCC4(CC3)CCN(c3cccc(C5CCC(=O)NC5=O)c3)CC4)cc2)C1] |
| 171 | 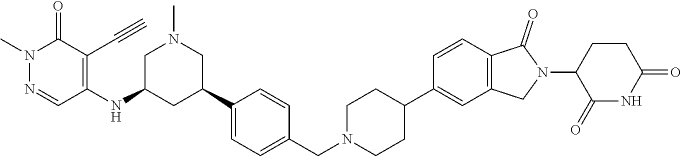 [SMILES: C#Cc1c(N[C@@H]2C[C@H](c3ccc(CN4CCC(c5ccc6c(c5)CN(C5CCC(=O)NC5=O)C6=O)CC4)cc3)CN(C)C2)cnn(C)c1=O] |
| 171a | 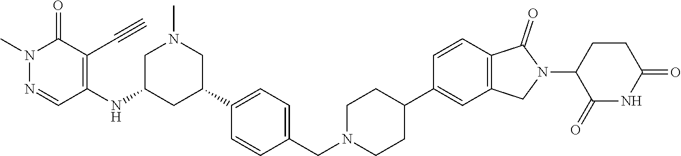 [SMILES: C#Cc1c(N[C@H]2C[C@@H](c3ccc(CN4CCC(c5ccc6c(c5)CN(C5CCC(=O)NC5=O)C6=O)CC4)cc3)CN(C)C2)cnn(C)c1=O] |
| 171b | 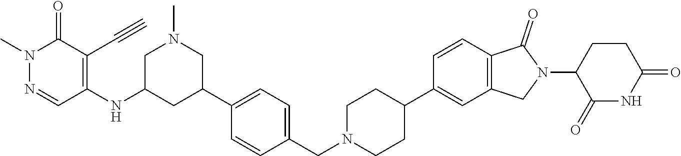 [SMILES: C#Cc1c(NC2CC(c3ccc(CN4CCC(c5ccc6c(c5)CN(C5CCC(=O)NC5=O)C6=O)CC4)cc3)CN(C)C2)cnn(C)c1=O] |
| 172 | 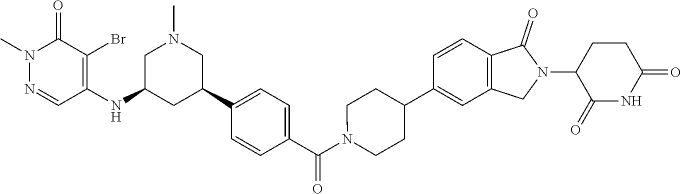 [SMILES: CN1C[C@H](Nc2cnn(C)c(=O)c2Br)C[C@H](c2ccc(C(=O)N3CCC(c4ccc5c(c4)CN(C4CCC(=O)NC4=O)C5=O)CC3)cc2)C1] |
| 172a | 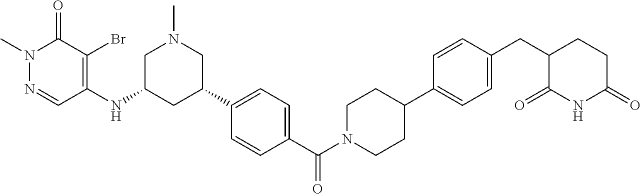 [SMILES: CN1C[C@@H](Nc2cnn(C)c(=O)c2Br)C[C@@H](c2ccc(C(=O)N3CCC(c4ccc(CC5CCC(=O)NC5=O)cc4)CC3)cc2)C1] |
| 172b | 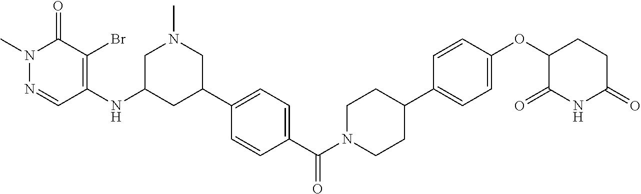 [SMILES: CN1CC(Nc2cnn(C)c(=O)c2Br)CC(c2ccc(C(=O)N3CCC(c4ccc(OC5CCC(=O)NC5=O)cc4)CC3)cc2)C1] |
| 173 | 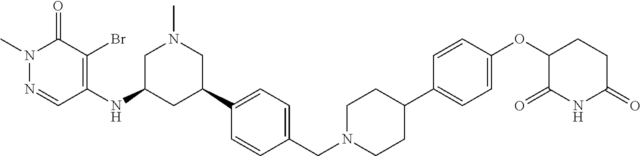 [SMILES: CN1C[C@H](Nc2cnn(C)c(=O)c2Br)C[C@H](c2ccc(CN3CCC(c4ccc(OC5CCC(=O)NC5=O)cc4)CC3)cc2)C1] |
| 173a | 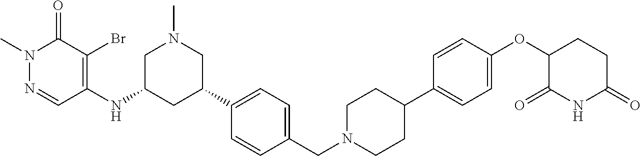 [SMILES: CN1C[C@@H](Nc2cnn(C)c(=O)c2Br)C[C@@H](c2ccc(CN3CCC(c4ccc(OC5CCC(=O)NC5=O)cc4)CC3)cc2)C1] |
| 173b | 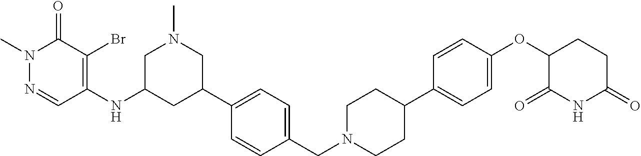 [SMILES: CN1CC(Nc2cnn(C)c(=O)c2Br)CC(c2ccc(CN3CCC(c4ccc(OC5CCC(=O)NC5=O)cc4)CC3)cc2)C1] |
| 174 | 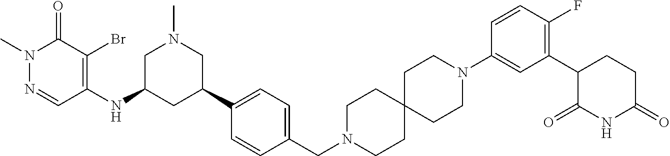 [SMILES: CN1C[C@H](Nc2cnn(C)c(=O)c2Br)C[C@H](c2ccc(CN3CCC4(CC3)CCN(c3ccc(F)c(C5CCC(=O)NC5=O)c3)CC4)cc2)C1] |
| 174a | 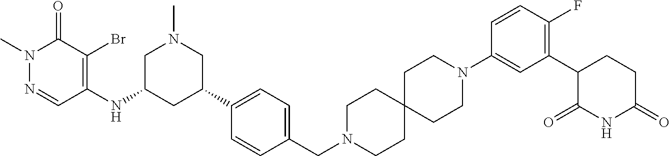 [SMILES: CN1C[C@@H](Nc2cnn(C)c(=O)c2Br)C[C@@H](c2ccc(CN3CCC4(CC3)CCN(c3ccc(F)c(C5CCC(=O)NC5=O)c3)CC4)cc2)C1] |
| 174b | 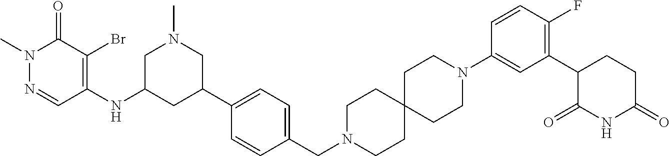 [SMILES: CN1CC(Nc2cnn(C)c(=O)c2Br)CC(c2ccc(CN3CCC4(CC3)CCN(c3ccc(F)c(C5CCC(=O)NC5=O)c3)CC4)cc2)C1] |
| 175 | 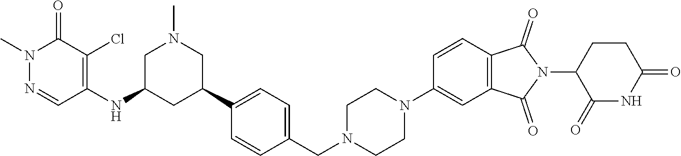 [SMILES: CN1C[C@H](Nc2cnn(C)c(=O)c2Cl)C[C@H](c2ccc(CN3CCN(c4ccc5c(c4)C(=O)N(C4CCC(=O)NC4=O)C5=O)CC3)cc2)C1] |
| 175a | 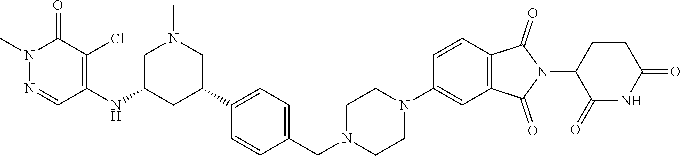 [SMILES: CN1C[C@@H](Nc2cnn(C)c(=O)c2Cl)C[C@@H](c2ccc(CN3CCN(c4ccc5c(c4)C(=O)N(C4CCC(=O)NC4=O)C5=O)CC3)cc2)C1] |
| 175b | 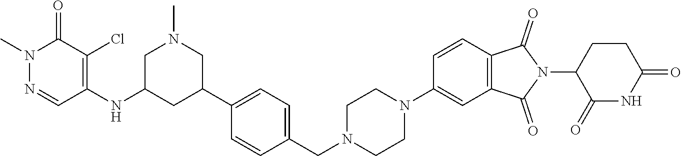 [SMILES: CN1CC(Nc2cnn(C)c(=O)c2Cl)CC(c2ccc(CN3CCN(c4ccc5c(c4)C(=O)N(C4CCC(=O)NC4=O)C5=O)CC3)cc2)C1] |
| 176 | 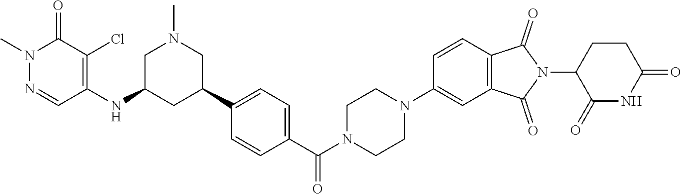 [SMILES: CN1C[C@H](Nc2cnn(C)c(=O)c2Cl)C[C@H](c2ccc(C(=O)N3CCN(c4ccc5c(c4)C(=O)N(C4CCC(=O)NC4=O)C5=O)CC3)cc2)C1] |
| 176a | 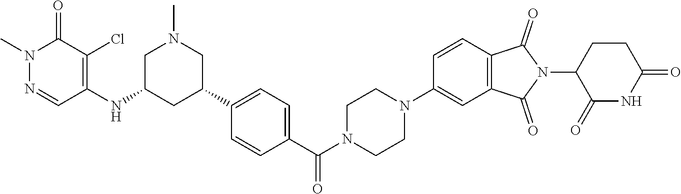 [SMILES: CN1C[C@@H](Nc2cnn(C)c(=O)c2Cl)C[C@@H](c2ccc(C(=O)N3CCN(c4ccc5c(c4)C(=O)N(C4CCC(=O)NC4=O)C5=O)CC3)cc2)C1] |
| 176b | 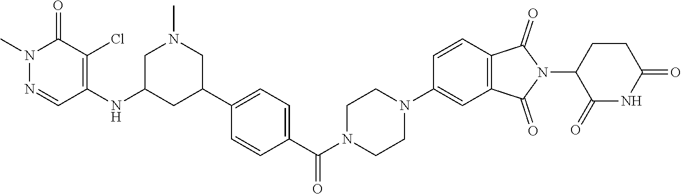 [SMILES: CN1CC(Nc2cnn(C)c(=O)c2Cl)CC(c2ccc(C(=O)N3CCN(c4ccc5c(c4)C(=O)N(C4CCC(=O)NC4=O)C5=O)CC3)cc2)C1] |
| 177 | 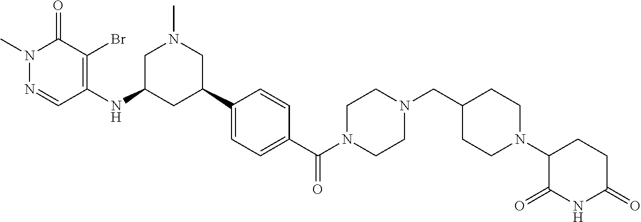 [SMILES: CN1C[C@H](Nc2cnn(C)c(=O)c2Br)C[C@H](c2ccc(C(=O)N3CCN(CC4CCN(C5CCC(=O)NC5=O)CC4)CC3)cc2)C1] |
| 177a | 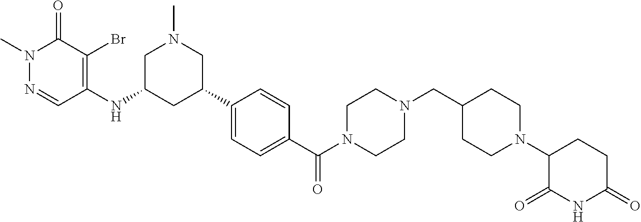 [SMILES: CN1C[C@@H](Nc2cnn(C)c(=O)c2Br)C[C@@H](c2ccc(C(=O)N3CCN(CC4CCN(C5CCC(=O)NC5=O)CC4)CC3)cc2)C1] |
| 177b | 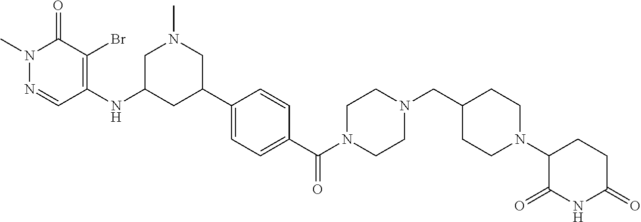 [SMILES: CN1CC(Nc2cnn(C)c(=O)c2Br)CC(c2ccc(C(=O)N3CCN(CC4CCN(C5CCC(=O)NC5=O)CC4)CC3)cc2)C1] |
| 178 | 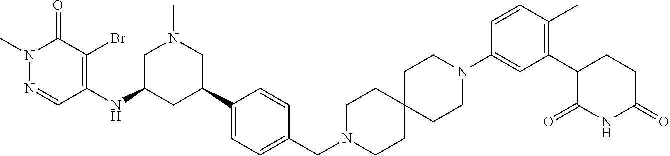 [SMILES: Cc1ccc(N2CCC3(CCN(Cc4ccc([C@H]5C[C@@H](Nc6cnn(C)c(=O)c6Br)CN(C)C5)cc4)CC3)CC2)cc1C1CCC(=O)NC1=O] |
| 178a | 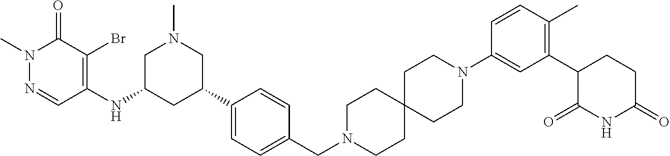 [SMILES: Cc1ccc(N2CCC3(CCN(Cc4ccc([C@@H]5C[C@H](Nc6cnn(C)c(=O)c6Br)CN(C)C5)cc4)CC3)CC2)cc1C1CCC(=O)NC1=O] |
| 178b | 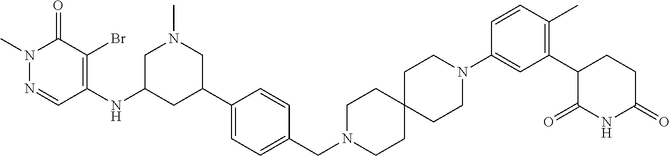 [SMILES: Cc1ccc(N2CCC3(CCN(Cc4ccc(C5CC(Nc6cnn(C)c(=O)c6Br)CN(C)C5)cc4)CC3)CC2)cc1C1CCC(=O)NC1=O] |
| 179 | 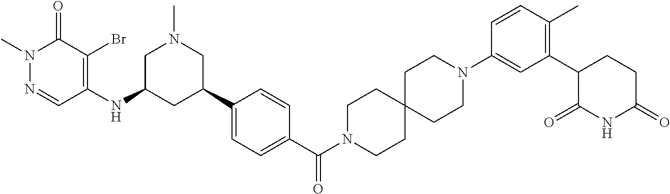 [SMILES: Cc1ccc(N2CCC3(CCN(C(=O)c4ccc([C@H]5C[C@@H](Nc6cnn(C)c(=O)c6Br)CN(C)C5)cc4)CC3)CC2)cc1C1CCC(=O)NC1=O] |
| 179a | 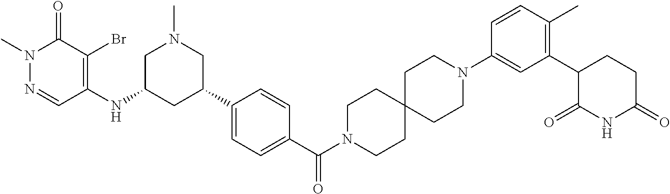 [SMILES: Cc1ccc(N2CCC3(CCN(C(=O)c4ccc([C@@H]5C[C@H](Nc6cnn(C)c(=O)c6Br)CN(C)C5)cc4)CC3)CC2)cc1C1CCC(=O)NC1=O] |
| 179b | 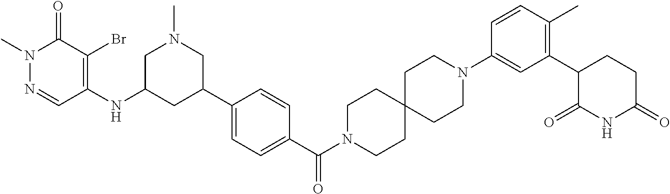 [SMILES: Cc1ccc(N2CCC3(CCN(C(=O)c4ccc(C5CC(Nc6cnn(C)c(=O)c6Br)CN(C)C5)cc4)CC3)CC2)cc1C1CCC(=O)NC1=O] |
| 180 | 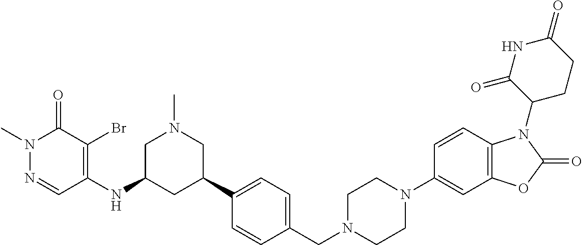 [SMILES: CN1C[C@H](Nc2cnn(C)c(=O)c2Br)C[C@H](c2ccc(CN3CCN(c4ccc5c(c4)oc(=O)n5C4CCC(=O)NC4=O)CC3)cc2)C1] |
| 180a | 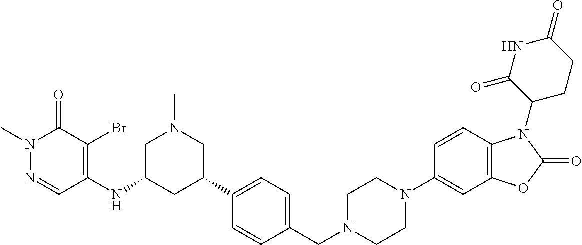 [SMILES: CN1C[C@@H](Nc2cnn(C)c(=O)c2Br)C[C@@H](c2ccc(CN3CCN(c4ccc5c(c4)oc(=O)n5C4CCC(=O)NC4=O)CC3)cc2)C1] |
| 180b | 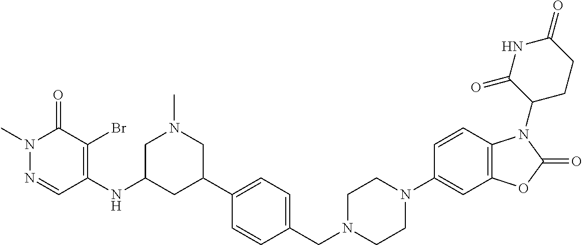 [SMILES: CN1CC(Nc2cnn(C)c(=O)c2Br)CC(c2ccc(CN3CCN(c4ccc5c(c4)oc(=O)n5C4CCC(=O)NC4=O)CC3)cc2)C1] |
| 181 | 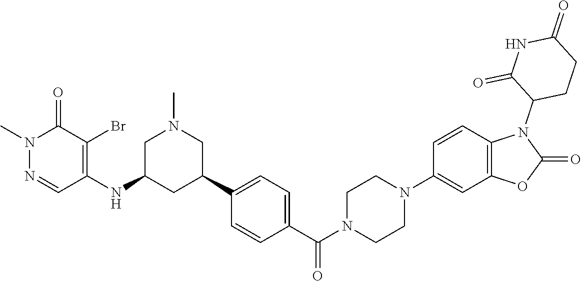 [SMILES: CN1C[C@H](Nc2cnn(C)c(=O)c2Br)C[C@H](c2ccc(C(=O)N3CCN(c4ccc5c(c4)oc(=O)n5C4CCC(=O)NC4=O)CC3)cc2)C1] |
| 181a | 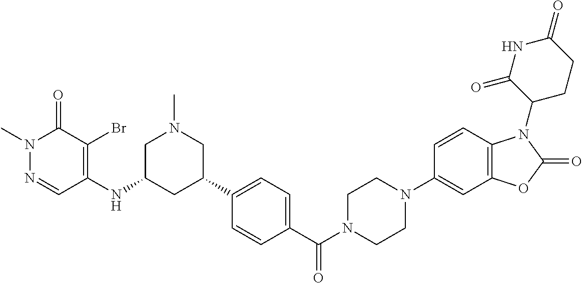 [SMILES: CN1C[C@@H](Nc2cnn(C)c(=O)c2Br)C[C@@H](c2ccc(C(=O)N3CCN(c4ccc5c(c4)oc(=O)n5C4CCC(=O)NC4=O)CC3)cc2)C1] |
| 181b | 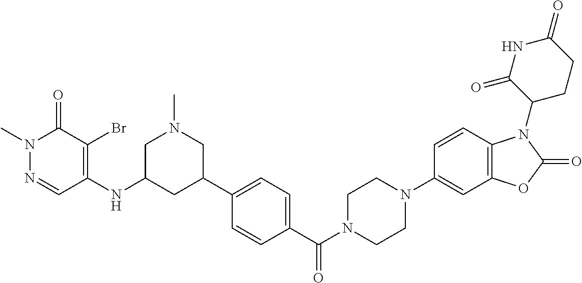 [SMILES: CN1CC(Nc2cnn(C)c(=O)c2Br)CC(c2ccc(C(=O)N3CCN(c4ccc5c(c4)oc(=O)n5C4CCC(=O)NC4=O)CC3)cc2)C1] |
| 182 | 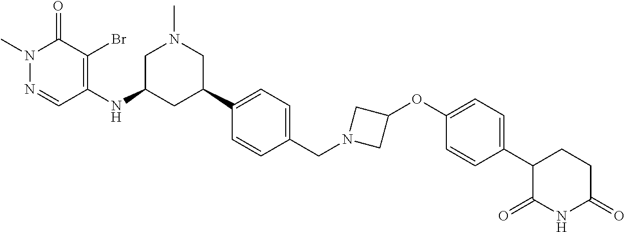 [SMILES: CN1C[C@H](Nc2cnn(C)c(=O)c2Br)C[C@H](c2ccc(CN3CC(Oc4ccc(C5CCC(=O)NC5=O)cc4)C3)cc2)C1] |
| 182a | 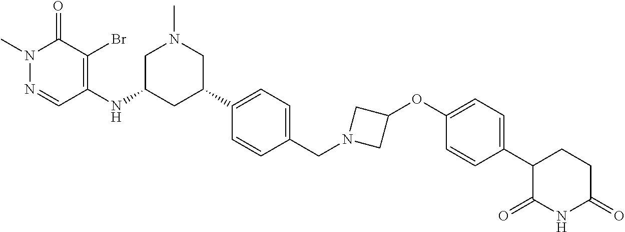 [SMILES: CN1C[C@@H](Nc2cnn(C)c(=O)c2Br)C[C@@H](c2ccc(CN3CC(Oc4ccc(C5CCC(=O)NC5=O)cc4)C3)cc2)C1] |
| 182b | 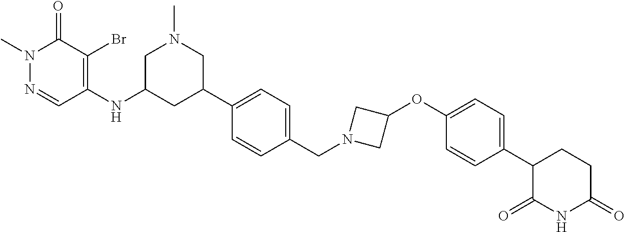 [SMILES: CN1CC(Nc2cnn(C)c(=O)c2Br)CC(c2ccc(CN3CC(Oc4ccc(C5CCC(=O)NC5=O)cc4)C3)cc2)C1] |
| 183 | 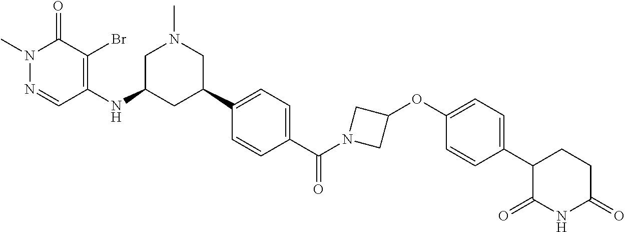 [SMILES: CN1C[C@H](Nc2cnn(C)c(=O)c2Br)C[C@H](c2ccc(C(=O)N3CC(Oc4ccc(C5CCC(=O)NC5=O)cc4)C3)cc2)C1] |
| 183a | 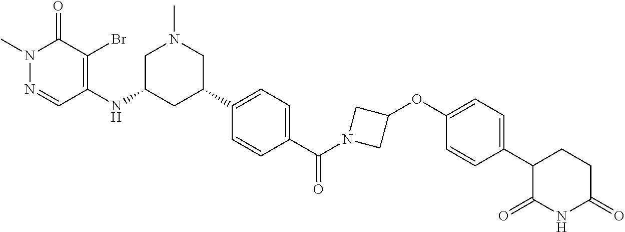 [SMILES: CN1C[C@@H](Nc2cnn(C)c(=O)c2Br)C[C@@H](c2ccc(C(=O)N3CC(Oc4ccc(C5CCC(=O)NC5=O)cc4)C3)cc2)C1] |
| 183b | 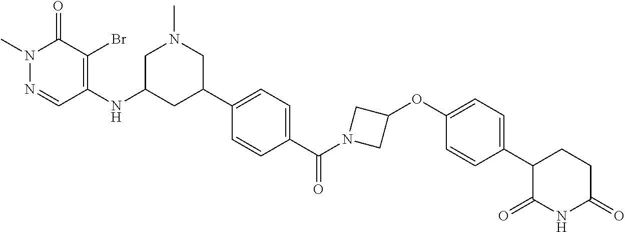 [SMILES: CN1CC(Nc2cnn(C)c(=O)c2Br)CC(c2ccc(C(=O)N3CC(Oc4ccc(C5CCC(=O)NC5=O)cc4)C3)cc2)C1] |
| 184 | 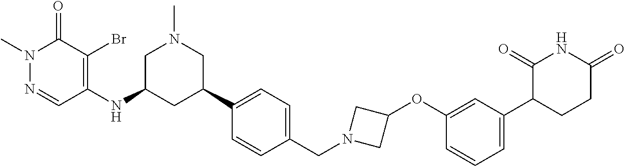 [SMILES: CN1C[C@H](Nc2cnn(C)c(=O)c2Br)C[C@H](c2ccc(CN3CC(Oc4cccc(C5CCC(=O)NC5=O)c4)C3)cc2)C1] |
| 184a | 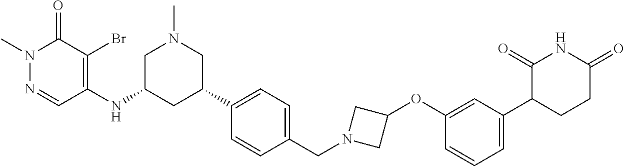 [SMILES: CN1C[C@@H](Nc2cnn(C)c(=O)c2Br)C[C@@H](c2ccc(CN3CC(Oc4cccc(C5CCC(=O)NC5=O)c4)C3)cc2)C1] |
| 184b | 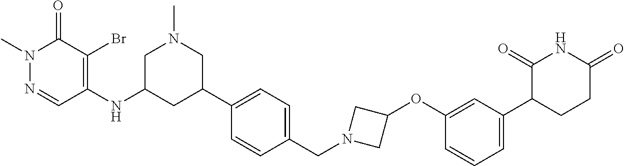 [SMILES: CN1CC(Nc2cnn(C)c(=O)c2Br)CC(c2ccc(CN3CC(Oc4cccc(C5CCC(=O)NC5=O)c4)C3)cc2)C1] |
| 185 | 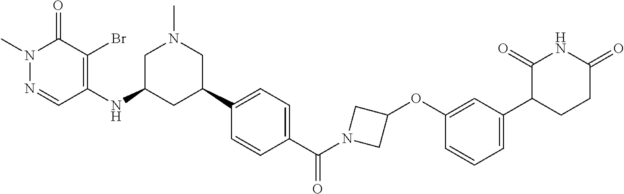 [SMILES: CN1C[C@H](Nc2cnn(C)c(=O)c2Br)C[C@H](c2ccc(C(=O)N3CC(Oc4cccc(C5CCC(=O)NC5=O)c4)C3)cc2)C1] |
| 185a | 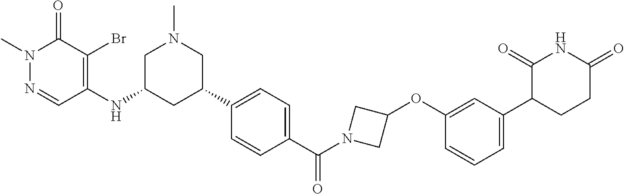 [SMILES: CN1C[C@@H](Nc2cnn(C)c(=O)c2Br)C[C@@H](c2ccc(C(=O)N3CC(Oc4cccc(C5CCC(=O)NC5=O)c4)C3)cc2)C1] |
| 185b | 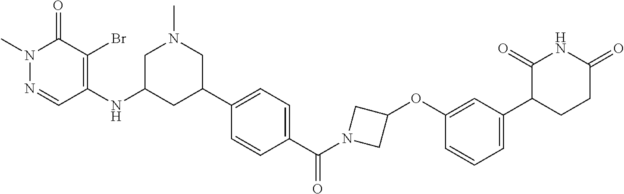 [SMILES: CN1CC(Nc2cnn(C)c(=O)c2Br)CC(c2ccc(C(=O)N3CC(Oc4cccc(C5CCC(=O)NC5=O)c4)C3)cc2)C1] |
| 186 | 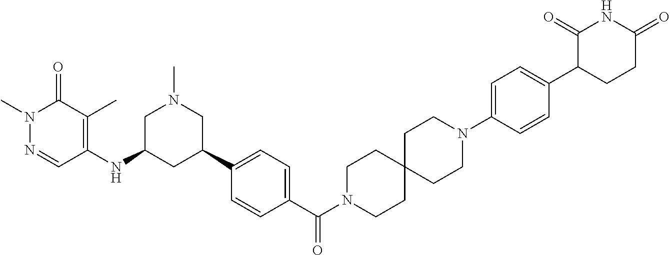 [SMILES: Cc1c(N[C@@H]2C[C@H](c3ccc(C(=O)N4CCC5(CC4)CCN(c4ccc(C6CCC(=O)NC6=O)cc4)CC5)cc3)CN(C)C2)cnn(C)c1=O] |
| 186a | 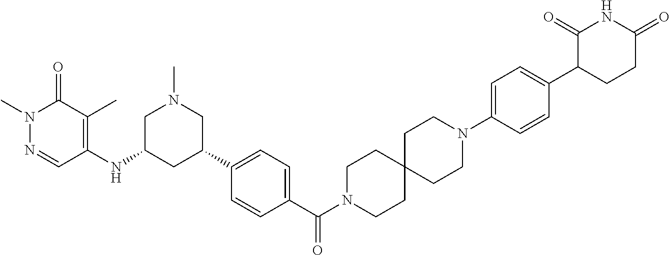 [SMILES: Cc1c(N[C@H]2C[C@@H](c3ccc(C(=O)N4CCC5(CC4)CCN(c4ccc(C6CCC(=O)NC6=O)cc4)CC5)cc3)CN(C)C2)cnn(C)c1=O] |
| 186b | 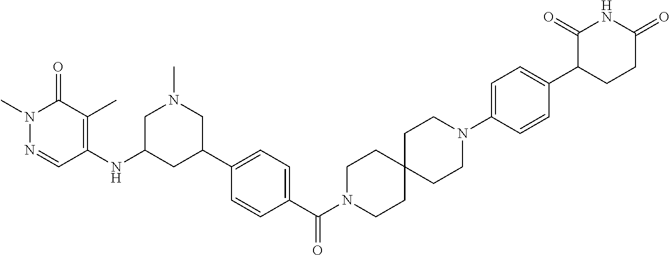 [SMILES: Cc1c(NC2CC(c3ccc(C(=O)N4CCC5(CC4)CCN(c4ccc(C6CCC(=O)NC6=O)cc4)CC5)cc3)CN(C)C2)cnn(C)c1=O] |
| 187 | 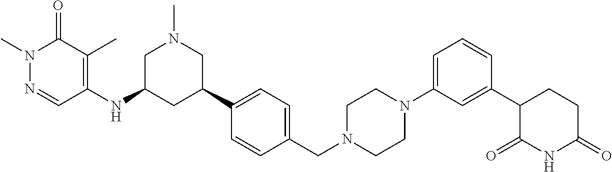 [SMILES: Cc1c(N[C@@H]2C[C@H](c3ccc(CN4CCN(c5cccc(C6CCC(=O)NC6=O)c5)CC4)cc3)CN(C)C2)cnn(C)c1=O] |
| 187a | 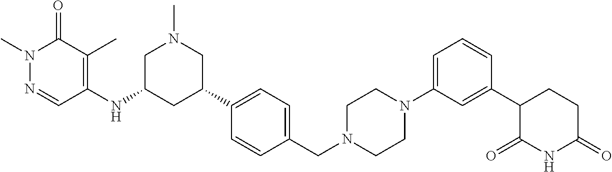 [SMILES: Cc1c(N[C@H]2C[C@@H](c3ccc(CN4CCN(c5cccc(C6CCC(=O)NC6=O)c5)CC4)cc3)CN(C)C2)cnn(C)c1=O] |
| 187b | 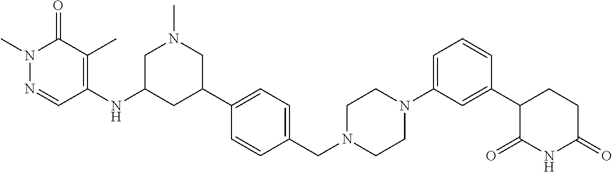 [SMILES: Cc1c(NC2CC(c3ccc(CN4CCN(c5cccc(C6CCC(=O)NC6=O)c5)CC4)cc3)CN(C)C2)cnn(C)c1=O] |
| 188 | 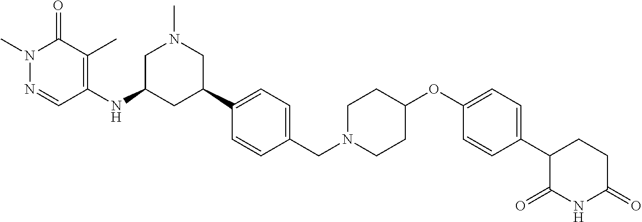 [SMILES: Cc1c(N[C@@H]2C[C@H](c3ccc(CN4CCC(Oc5ccc(C6CCC(=O)NC6=O)cc5)CC4)cc3)CN(C)C2)cnn(C)c1=O] |
| 188a | 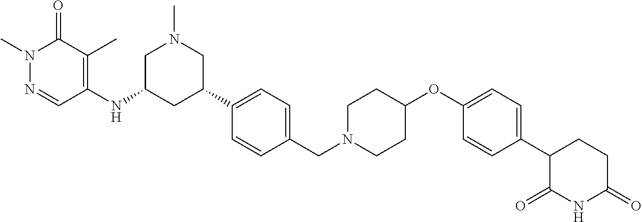 [SMILES: Cc1c(N[C@H]2C[C@@H](c3ccc(CN4CCC(Oc5ccc(C6CCC(=O)NC6=O)cc5)CC4)cc3)CN(C)C2)cnn(C)c1=O] |
| 188b | 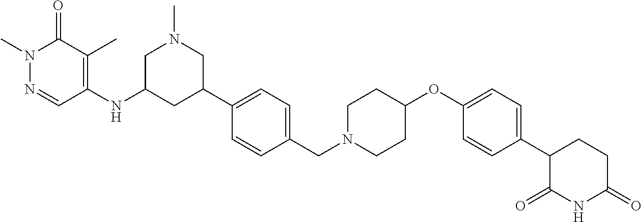 [SMILES: Cc1c(NC2CC(c3ccc(CN4CCC(Oc5ccc(C6CCC(=O)NC6=O)cc5)CC4)cc3)CN(C)C2)cnn(C)c1=O] |
| 189 | 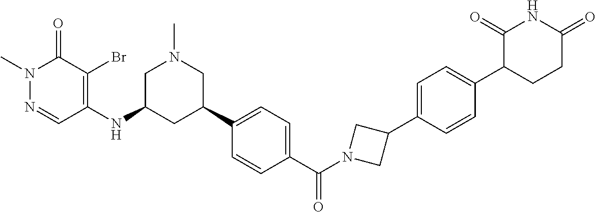 [SMILES: CN1C[C@H](Nc2cnn(C)c(=O)c2Br)C[C@H](c2ccc(C(=O)N3CC(c4ccc(C5CCC(=O)NC5=O)cc4)C3)cc2)C1] |
| 189a | 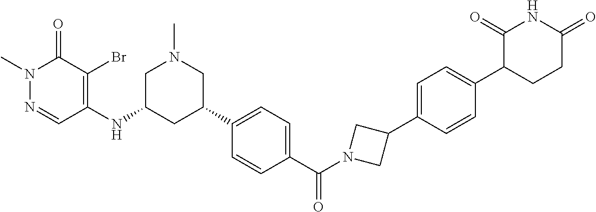 [SMILES: CN1C[C@@H](Nc2cnn(C)c(=O)c2Br)C[C@@H](c2ccc(C(=O)N3CC(c4ccc(C5CCC(=O)NC5=O)cc4)C3)cc2)C1] |
| 189b | 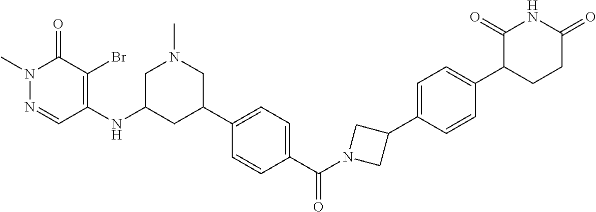 [SMILES: CN1CC(Nc2cnn(C)c(=O)c2Br)CC(c2ccc(C(=O)N3CC(c4ccc(C5CCC(=O)NC5=O)cc4)C3)cc2)C1] |
| 190 | 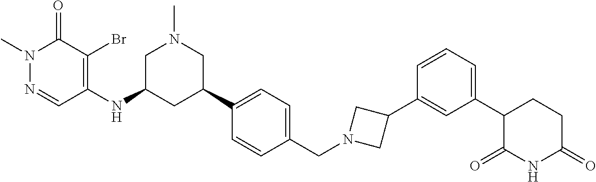 [SMILES: CN1C[C@H](Nc2cnn(C)c(=O)c2Br)C[C@H](c2ccc(CN3CC(c4cccc(C5CCC(=O)NC5=O)c4)C3)cc2)C1] |
| 190a | 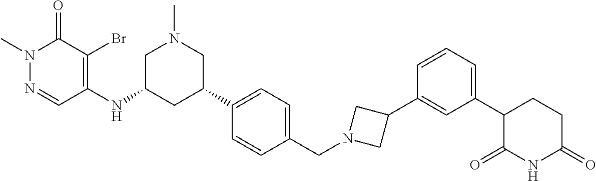 [SMILES: CN1C[C@@H](Nc2cnn(C)c(=O)c2Br)C[C@@H](c2ccc(CN3CC(c4cccc(C5CCC(=O)NC5=O)c4)C3)cc2)C1] |
| 190b | 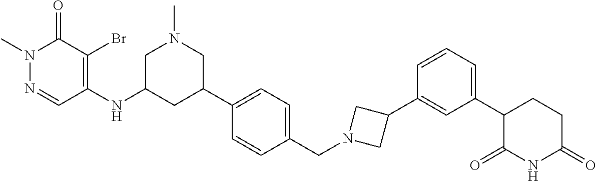 [SMILES: CN1CC(Nc2cnn(C)c(=O)c2Br)CC(c2ccc(CN3CC(c4cccc(C5CCC(=O)NC5=O)c4)C3)cc2)C1] |
| 191 | 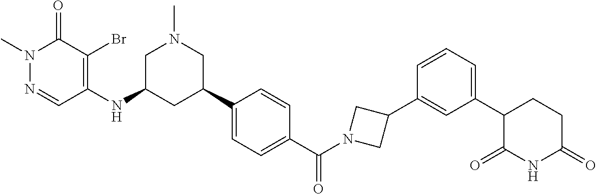 [SMILES: CN1C[C@H](Nc2cnn(C)c(=O)c2Br)C[C@H](c2ccc(C(=O)N3CC(c4cccc(C5CCC(=O)NC5=O)c4)C3)cc2)C1] |
| 191a | 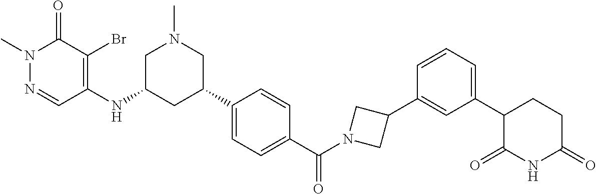 [SMILES: CN1C[C@@H](Nc2cnn(C)c(=O)c2Br)C[C@@H](c2ccc(C(=O)N3CC(c4cccc(C5CCC(=O)NC5=O)c4)C3)cc2)C1] |
| 191b | 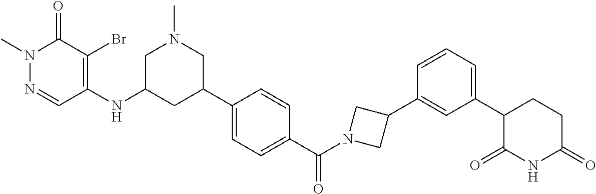 [SMILES: CN1CC(Nc2cnn(C)c(=O)c2Br)CC(c2ccc(C(=O)N3CC(c4cccc(C5CCC(=O)NC5=O)c4)C3)cc2)C1] |
| 192 | 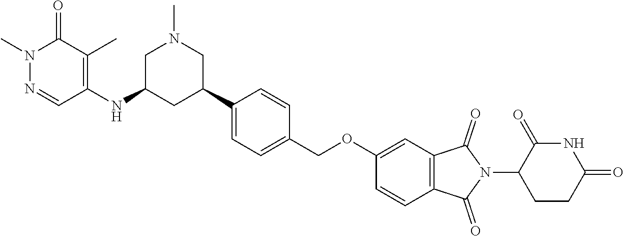 [SMILES: Cc1c(N[C@@H]2C[C@H](c3ccc(COc4ccc5c(c4)C(=O)N(C4CCC(=O)NC4=O)C5=O)cc3)CN(C)C2)cnn(C)c1=O] |
| 192a | 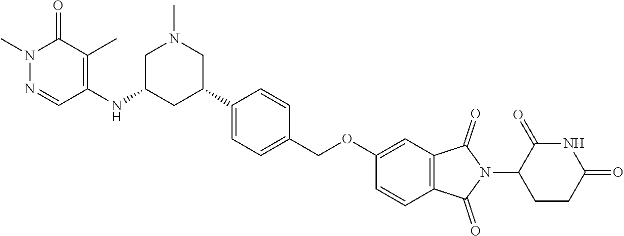 [SMILES: Cc1c(N[C@H]2C[C@@H](c3ccc(COc4ccc5c(c4)C(=O)N(C4CCC(=O)NC4=O)C5=O)cc3)CN(C)C2)cnn(C)c1=O] |
| 192b | 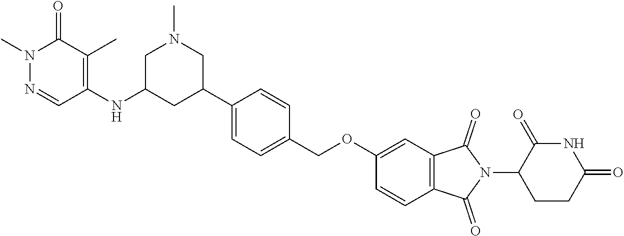 [SMILES: Cc1c(NC2CC(c3ccc(COc4ccc5c(c4)C(=O)N(C4CCC(=O)NC4=O)C5=O)cc3)CN(C)C2)cnn(C)c1=O] |
| 193 | 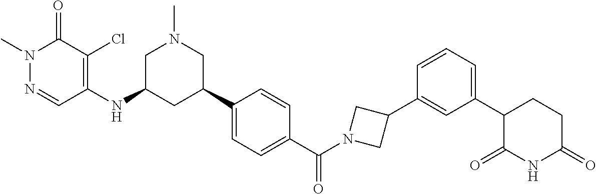 [SMILES: CN1C[C@H](Nc2cnn(C)c(=O)c2Cl)C[C@H](c2ccc(C(=O)N3CC(c4cccc(C5CCC(=O)NC5=O)c4)C3)cc2)C1] |
| 193a | 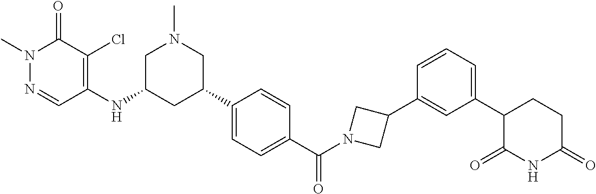 [SMILES: CN1C[C@@H](Nc2cnn(C)c(=O)c2Cl)C[C@@H](c2ccc(C(=O)N3CC(c4cccc(C5CCC(=O)NC5=O)c4)C3)cc2)C1] |
| 193b | 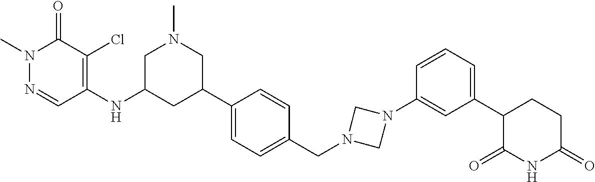 [SMILES: CN1CC(Nc2cnn(C)c(=O)c2Cl)CC(c2ccc(CN3CN(c4cccc(C5CCC(=O)NC5=O)c4)C3)cc2)C1] |
| 194 | 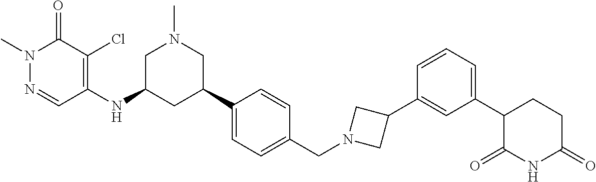 [SMILES: CN1C[C@H](Nc2cnn(C)c(=O)c2Cl)C[C@H](c2ccc(CN3CC(c4cccc(C5CCC(=O)NC5=O)c4)C3)cc2)C1] |
| 194a | 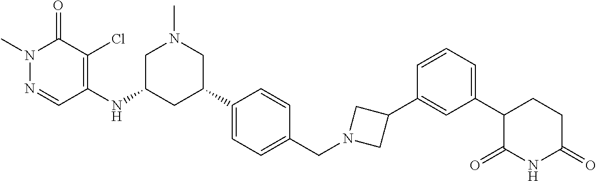 [SMILES: CN1C[C@@H](Nc2cnn(C)c(=O)c2Cl)C[C@@H](c2ccc(CN3CC(c4cccc(C5CCC(=O)NC5=O)c4)C3)cc2)C1] |
| 194b | 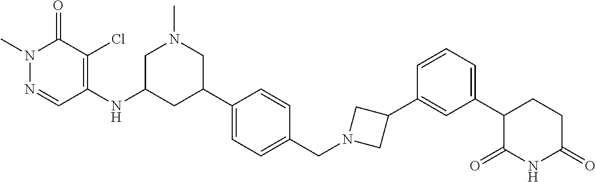 [SMILES: CN1CC(Nc2cnn(C)c(=O)c2Cl)CC(c2ccc(CN3CC(c4cccc(C5CCC(=O)NC5=O)c4)C3)cc2)C1] |
| 195 | 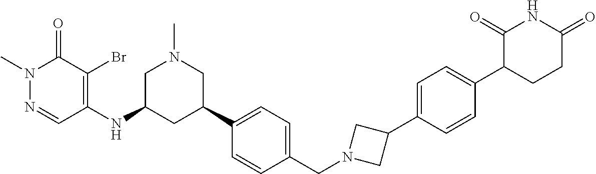 [SMILES: CN1C[C@H](Nc2cnn(C)c(=O)c2Br)C[C@H](c2ccc(CN3CC(c4ccc(C5CCC(=O)NC5=O)cc4)C3)cc2)C1] |
| 195a | 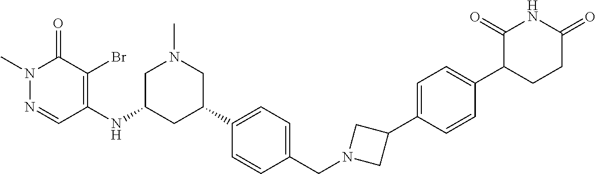 [SMILES: CN1C[C@@H](Nc2cnn(C)c(=O)c2Br)C[C@@H](c2ccc(CN3CC(c4ccc(C5CCC(=O)NC5=O)cc4)C3)cc2)C1] |
| 195b | 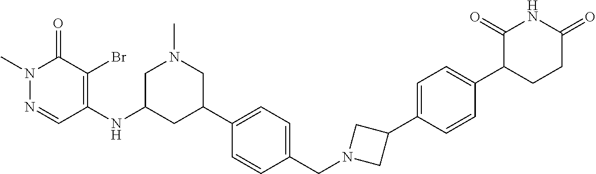 [SMILES: CN1CC(Nc2cnn(C)c(=O)c2Br)CC(c2ccc(CN3CC(c4ccc(C5CCC(=O)NC5=O)cc4)C3)cc2)C1] |
| 196 | 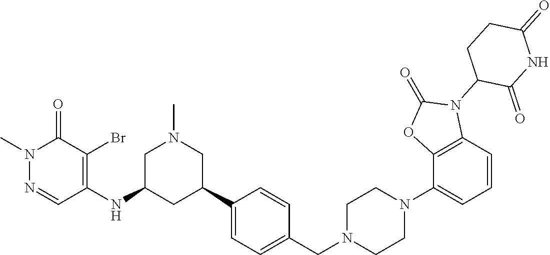 [SMILES: CN1C[C@H](Nc2cnn(C)c(=O)c2Br)C[C@H](c2ccc(CN3CCN(c4cccc5c4oc(=O)n5C4CCC(=O)NC4=O)CC3)cc2)C1] |
| 196a | 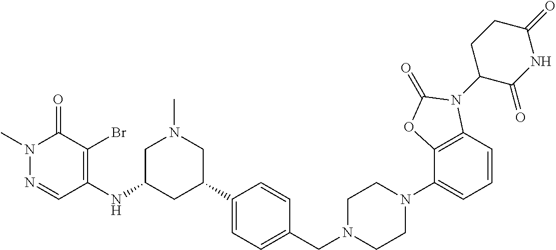 [SMILES: CN1C[C@@H](Nc2cnn(C)c(=O)c2Br)C[C@@H](c2ccc(CN3CCN(c4cccc5c4oc(=O)n5C4CCC(=O)NC4=O)CC3)cc2)C1] |
| 196b | 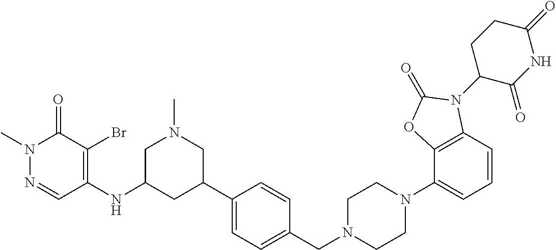 [SMILES: CN1CC(Nc2cnn(C)c(=O)c2Br)CC(c2ccc(CN3CCN(c4cccc5c4oc(=O)n5C4CCC(=O)NC4=O)CC3)cc2)C1] |
| 197 | 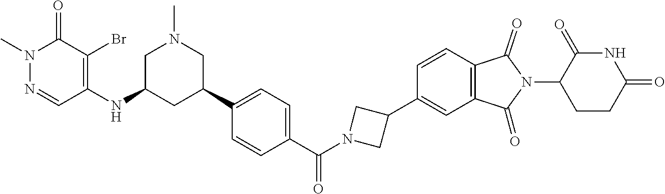 [SMILES: CN1C[C@H](Nc2cnn(C)c(=O)c2Br)C[C@H](c2ccc(C(=O)N3CC(c4ccc5c(c4)C(=O)N(C4CCC(=O)NC4=O)C5=O)C3)cc2)C1] |
| 197a | 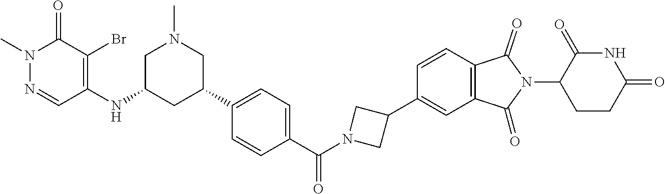 [SMILES: CN1C[C@@H](Nc2cnn(C)c(=O)c2Br)C[C@@H](c2ccc(C(=O)N3CC(c4ccc5c(c4)C(=O)N(C4CCC(=O)NC4=O)C5=O)C3)cc2)C1] |
| 197b | 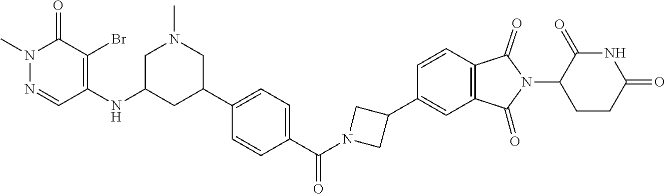 [SMILES: CN1CC(Nc2cnn(C)c(=O)c2Br)CC(c2ccc(C(=O)N3CC(c4ccc5c(c4)C(=O)N(C4CCC(=O)NC4=O)C5=O)C3)cc2)C1] |
| 198 | 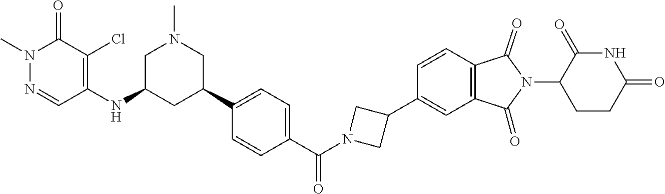 [SMILES: CN1C[C@H](Nc2cnn(C)c(=O)c2Cl)C[C@H](c2ccc(C(=O)N3CC(c4ccc5c(c4)C(=O)N(C4CCC(=O)NC4=O)C5=O)C3)cc2)C1] |
| 198a | 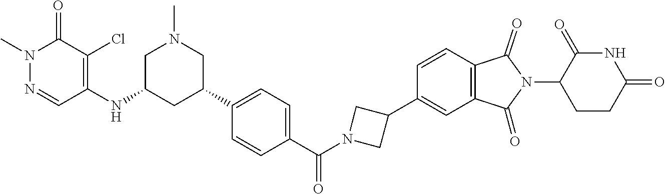 [SMILES: CN1C[C@@H](Nc2cnn(C)c(=O)c2Cl)C[C@@H](c2ccc(C(=O)N3CC(c4ccc5c(c4)C(=O)N(C4CCC(=O)NC4=O)C5=O)C3)cc2)C1] |
| 198b | 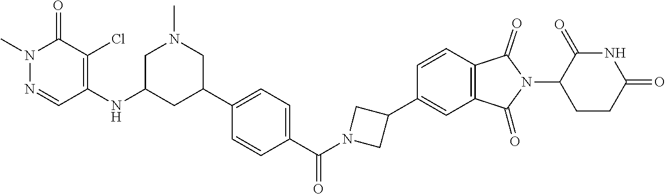 [SMILES: CN1CC(Nc2cnn(C)c(=O)c2Cl)CC(c2ccc(C(=O)N3CC(c4ccc5c(c4)C(=O)N(C4CCC(=O)NC4=O)C5=O)C3)cc2)C1] |
| 199 | 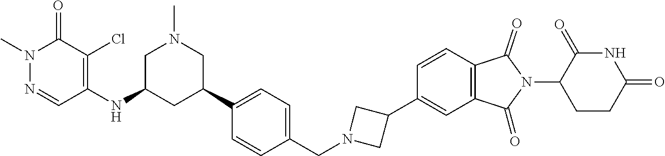 [SMILES: CN1C[C@H](Nc2cnn(C)c(=O)c2Cl)C[C@H](c2ccc(CN3CC(c4ccc5c(c4)C(=O)N(C4CCC(=O)NC4=O)C5=O)C3)cc2)C1] |
| 199a | 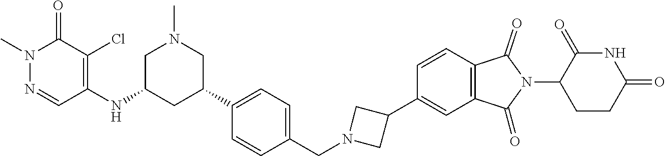 [SMILES: CN1C[C@@H](Nc2cnn(C)c(=O)c2Cl)C[C@@H](c2ccc(CN3CC(c4ccc5c(c4)C(=O)N(C4CCC(=O)NC4=O)C5=O)C3)cc2)C1] |
| 199b | 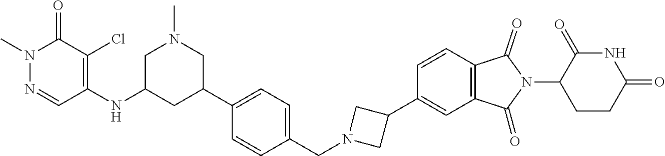 [SMILES: CN1CC(Nc2cnn(C)c(=O)c2Cl)CC(c2ccc(CN3CC(c4ccc5c(c4)C(=O)N(C4CCC(=O)NC4=O)C5=O)C3)cc2)C1] |
| 200 | 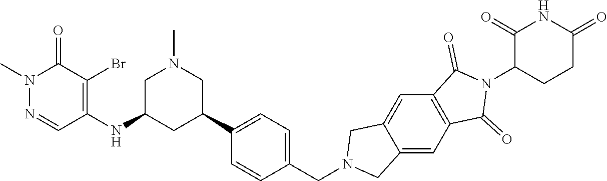 [SMILES: CN1C[C@H](Nc2cnn(C)c(=O)c2Br)C[C@H](c2ccc(CN3Cc4cc5c(cc4C3)C(=O)N(C3CCC(=O)NC3=O)C5=O)cc2)C1] |
| 200a | 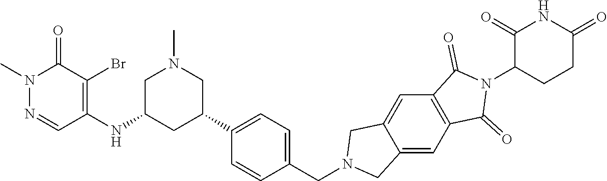 [SMILES: CN1C[C@@H](Nc2cnn(C)c(=O)c2Br)C[C@@H](c2ccc(CN3Cc4cc5c(cc4C3)C(=O)N(C3CCC(=O)NC3=O)C5=O)cc2)C1] |
| 200b | 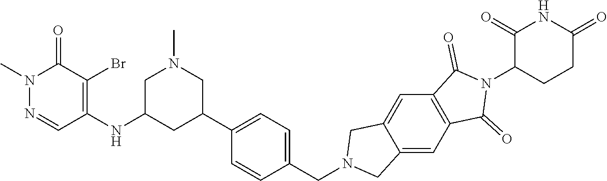 [SMILES: CN1CC(Nc2cnn(C)c(=O)c2Br)CC(c2ccc(CN3Cc4cc5c(cc4C3)C(=O)N(C3CCC(=O)NC3=O)C5=O)cc2)C1] |
| 201 | 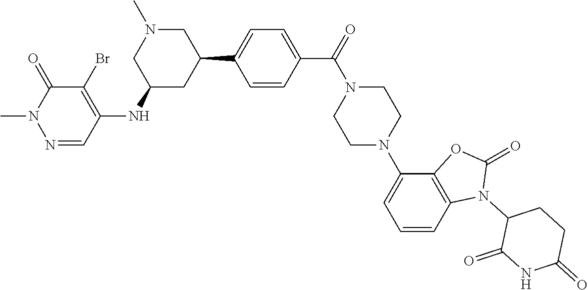 [SMILES: CN1C[C@H](Nc2cnn(C)c(=O)c2Br)C[C@H](c2ccc(C(=O)N3CCN(c4cccc5c4oc(=O)n5C4CCC(=O)NC4=O)CC3)cc2)C1] |
| 201a | 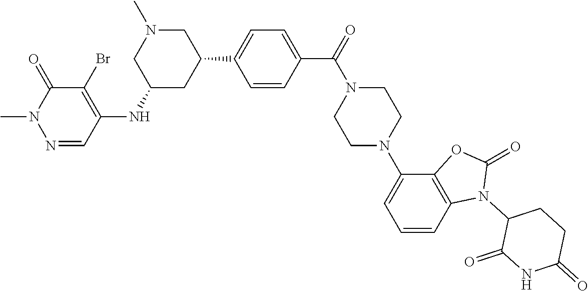 [SMILES: CN1C[C@@H](Nc2cnn(C)c(=O)c2Br)C[C@@H](c2ccc(C(=O)N3CCN(c4cccc5c4oc(=O)n5C4CCC(=O)NC4=O)CC3)cc2)C1] |
| 201b | 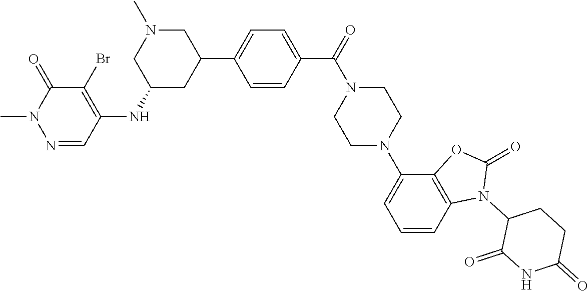 [SMILES: CN1CC(c2ccc(C(=O)N3CCN(c4cccc5c4oc(=O)n5C4CCC(=O)NC4=O)CC3)cc2)C[C@H](Nc2cnn(C)c(=O)c2Br)C1] |
| 202 | 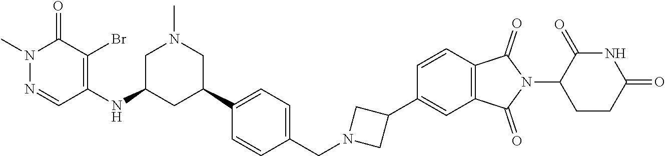 [SMILES: CN1C[C@H](Nc2cnn(C)c(=O)c2Br)C[C@H](c2ccc(CN3CC(c4ccc5c(c4)C(=O)N(C4CCC(=O)NC4=O)C5=O)C3)cc2)C1] |
| 202a | 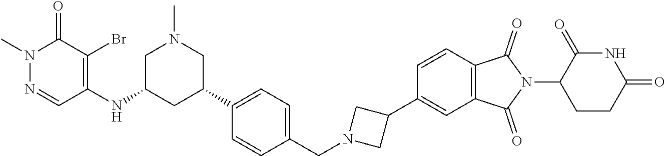 [SMILES: CN1C[C@@H](Nc2cnn(C)c(=O)c2Br)C[C@@H](c2ccc(CN3CC(c4ccc5c(c4)C(=O)N(C4CCC(=O)NC4=O)C5=O)C3)cc2)C1] |
| 202b | 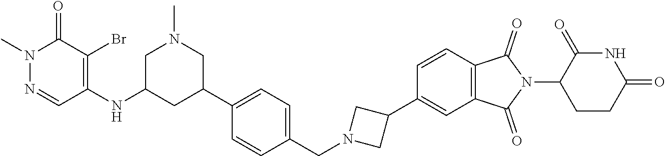 [SMILES: CN1CC(Nc2cnn(C)c(=O)c2Br)CC(c2ccc(CN3CC(c4ccc5c(c4)C(=O)N(C4CCC(=O)NC4=O)C5=O)C3)cc2)C1] |
| 203 | 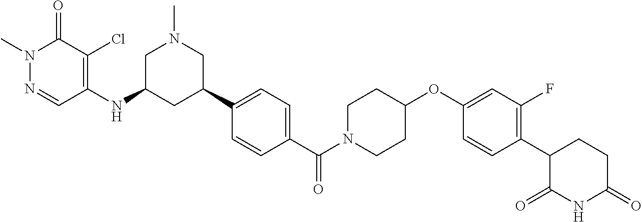 [SMILES: CN1C[C@H](Nc2cnn(C)c(=O)c2Cl)C[C@H](c2ccc(C(=O)N3CCC(Oc4ccc(C5CCC(=O)NC5=O)c(F)c4)CC3)cc2)C1] |
| 203a | 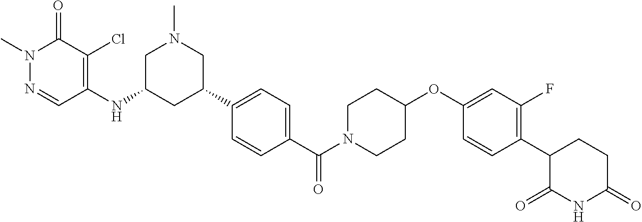 [SMILES: CN1C[C@@H](Nc2cnn(C)c(=O)c2Cl)C[C@@H](c2ccc(C(=O)N3CCC(Oc4ccc(C5CCC(=O)NC5=O)c(F)c4)CC3)cc2)C1] |
| 203b | 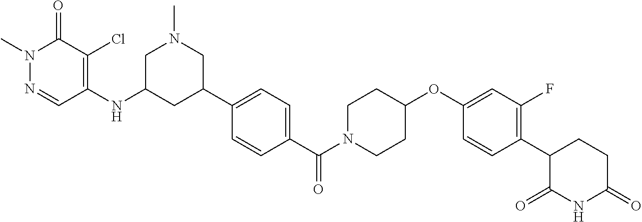 [SMILES: CN1CC(Nc2cnn(C)c(=O)c2Cl)CC(c2ccc(C(=O)N3CCC(Oc4ccc(C5CCC(=O)NC5=O)c(F)c4)CC3)cc2)C1] |
| 204 | 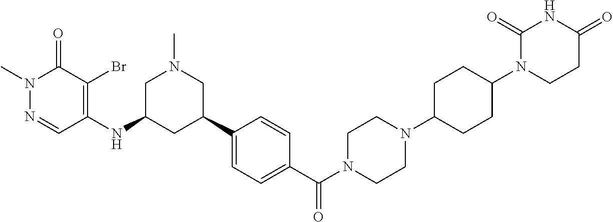 [SMILES: CN1C[C@H](Nc2cnn(C)c(=O)c2Br)C[C@H](c2ccc(C(=O)N3CCN(C4CCC(N5CCC(=O)NC5=O)CC4)CC3)cc2)C1] |
| 204a | 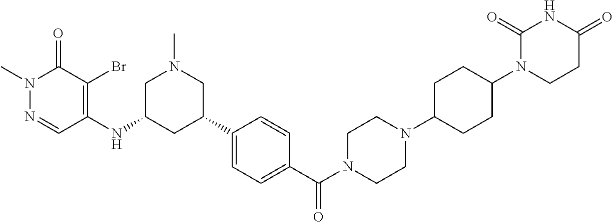 [SMILES: CN1C[C@@H](Nc2cnn(C)c(=O)c2Br)C[C@@H](c2ccc(C(=O)N3CCN(C4CCC(N5CCC(=O)NC5=O)CC4)CC3)cc2)C1] |
| 204b | 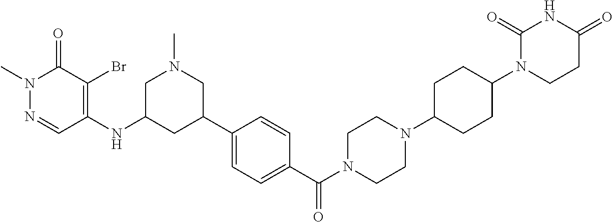 [SMILES: CN1CC(Nc2cnn(C)c(=O)c2Br)CC(c2ccc(C(=O)N3CCN(C4CCC(N5CCC(=O)NC5=O)CC4)CC3)cc2)C1] |
| 205 | 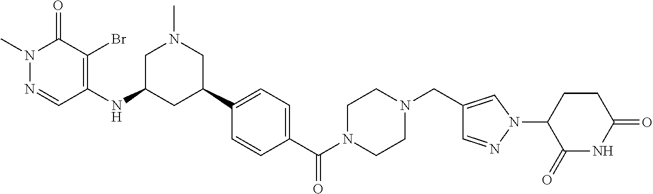 [SMILES: CN1C[C@H](Nc2cnn(C)c(=O)c2Br)C[C@H](c2ccc(C(=O)N3CCN(Cc4cnn(C5CCC(=O)NC5=O)c4)CC3)cc2)C1] |
| 205a | 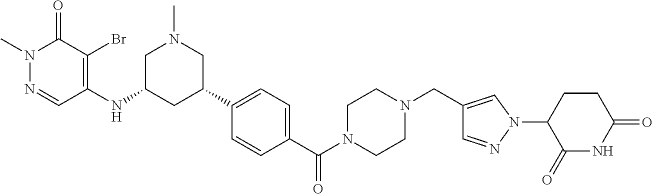 [SMILES: CN1C[C@@H](Nc2cnn(C)c(=O)c2Br)C[C@@H](c2ccc(C(=O)N3CCN(Cc4cnn(C5CCC(=O)NC5=O)c4)CC3)cc2)C1] |
| 205b | 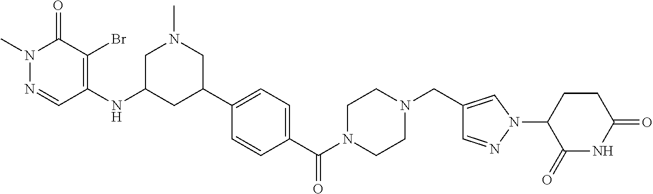 [SMILES: CN1CC(Nc2cnn(C)c(=O)c2Br)CC(c2ccc(C(=O)N3CCN(Cc4cnn(C5CCC(=O)NC5=O)c4)CC3)cc2)C1] |
| 206 | 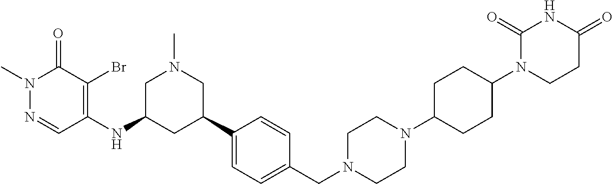 [SMILES: CN1C[C@H](Nc2cnn(C)c(=O)c2Br)C[C@H](c2ccc(CN3CCN(C4CCC(N5CCC(=O)NC5=O)CC4)CC3)cc2)C1] |
| 206a | 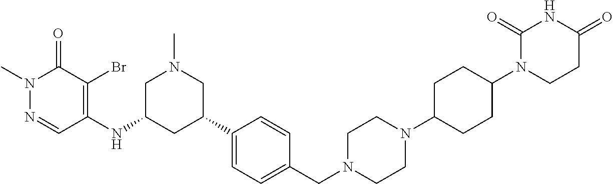 [SMILES: CN1C[C@@H](Nc2cnn(C)c(=O)c2Br)C[C@@H](c2ccc(CN3CCN(C4CCC(N5CCC(=O)NC5=O)CC4)CC3)cc2)C1] |
| 206b | 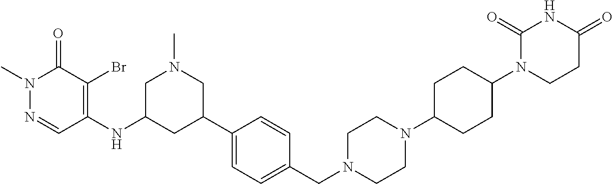 [SMILES: CN1CC(Nc2cnn(C)c(=O)c2Br)CC(c2ccc(CN3CCN(C4CCC(N5CCC(=O)NC5=O)CC4)CC3)cc2)C1] |
| 207 | 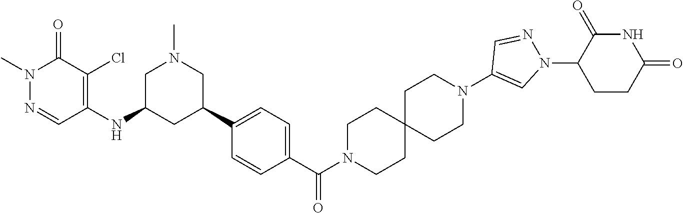 [SMILES: CN1C[C@H](Nc2cnn(C)c(=O)c2Cl)C[C@H](c2ccc(C(=O)N3CCC4(CC3)CCN(c3cnn(C5CCC(=O)NC5=O)c3)CC4)cc2)C1] |
| 207a | 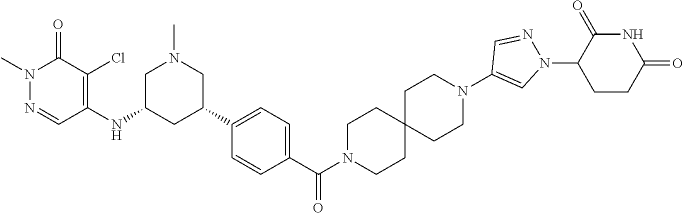 [SMILES: CN1C[C@@H](Nc2cnn(C)c(=O)c2Cl)C[C@@H](c2ccc(C(=O)N3CCC4(CC3)CCN(c3cnn(C5CCC(=O)NC5=O)c3)CC4)cc2)C1] |
| 207b | 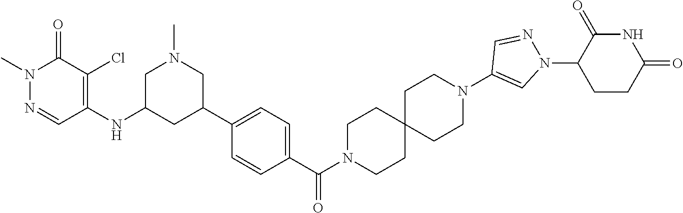 [SMILES: CN1CC(Nc2cnn(C)c(=O)c2Cl)CC(c2ccc(C(=O)N3CCC4(CC3)CCN(c3cnn(C5CCC(=O)NC5=O)c3)CC4)cc2)C1] |
| 208 | 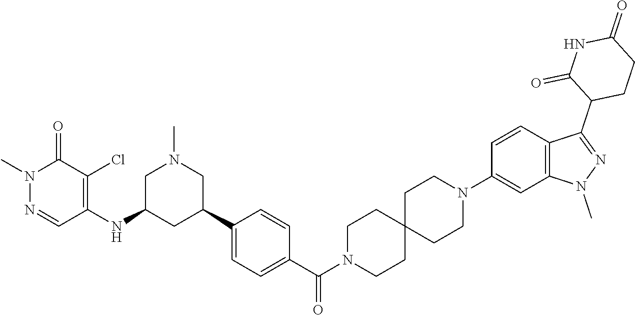 [SMILES: CN1C[C@H](Nc2cnn(C)c(=O)c2Cl)C[C@H](c2ccc(C(=O)N3CCC4(CC3)CCN(c3ccc5c(C6CCC(=O)NC6=O)nn(C)c5c3)CC4)cc2)C1] |
| 208-1 | 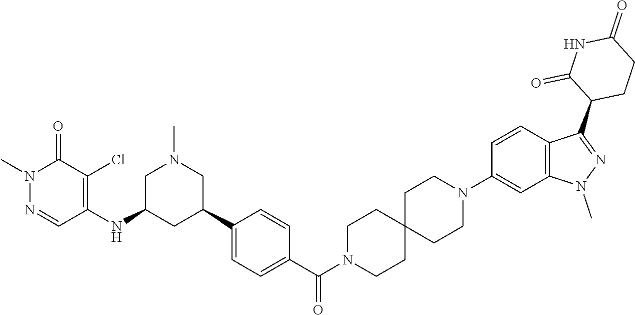 [SMILES: CN1C[C@H](Nc2cnn(C)c(=O)c2Cl)C[C@H](c2ccc(C(=O)N3CCC4(CC3)CCN(c3ccc5c([C@@H]6CCC(=O)NC6=O)nn(C)c5c3)CC4)cc2)C1] |
| 208-2 | 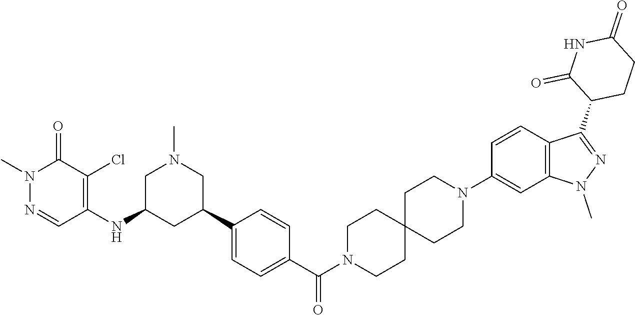 [SMILES: CN1C[C@H](Nc2cnn(C)c(=O)c2Cl)C[C@H](c2ccc(C(=O)N3CCC4(CC3)CCN(c3ccc5c([C@H]6CCC(=O)NC6=O)nn(C)c5c3)CC4)cc2)C1] |
| 208a | 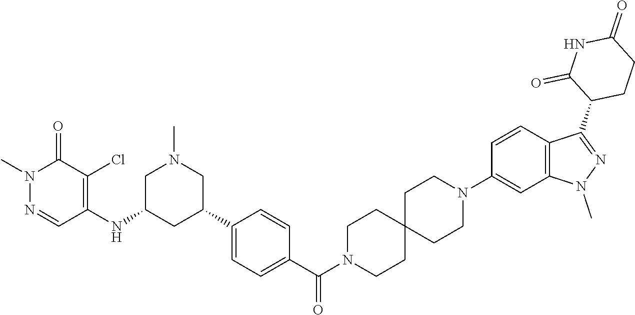 [SMILES: CN1C[C@@H](Nc2cnn(C)c(=O)c2Cl)C[C@@H](c2ccc(C(=O)N3CCC4(CC3)CCN(c3ccc5c([C@H]6CCC(=O)NC6=O)nn(C)c5c3)CC4)cc2)C1] |
| 208b | 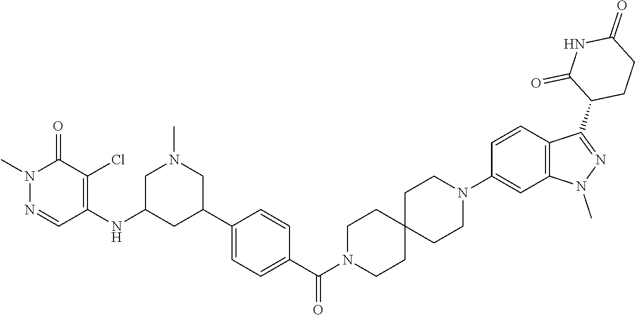 [SMILES: CN1CC(Nc2cnn(C)c(=O)c2Cl)CC(c2ccc(C(=O)N3CCC4(CC3)CCN(c3ccc5c([C@H]6CCC(=O)NC6=O)nn(C)c5c3)CC4)cc2)C1] |
| 209 | 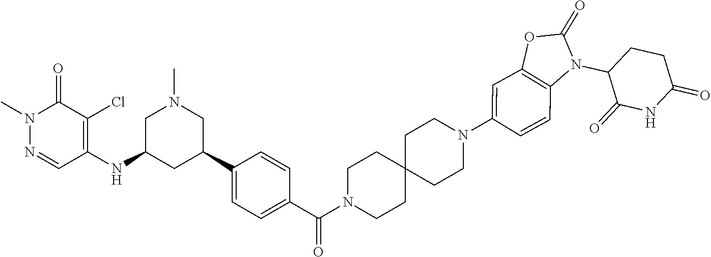 [SMILES: CN1C[C@H](Nc2cnn(C)c(=O)c2Cl)C[C@H](c2ccc(C(=O)N3CCC4(CC3)CCN(c3ccc5c(c3)oc(=O)n5C3CCC(=O)NC3=O)CC4)cc2)C1] |
| 209a | 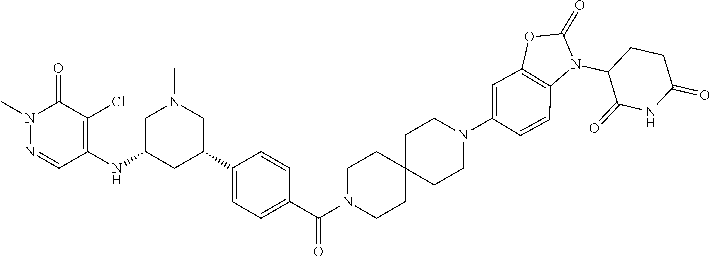 [SMILES: CN1C[C@@H](Nc2cnn(C)c(=O)c2Cl)C[C@@H](c2ccc(C(=O)N3CCC4(CC3)CCN(c3ccc5c(c3)oc(=O)n5C3CCC(=O)NC3=O)CC4)cc2)C1] |
| 209b | 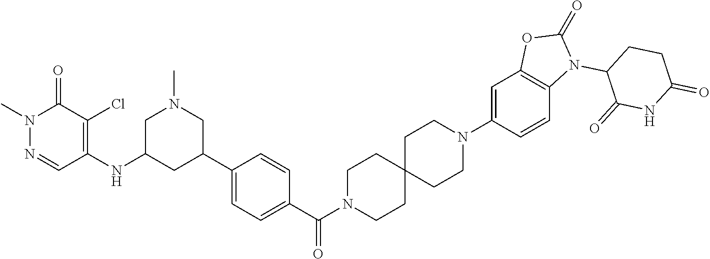 [SMILES: CN1CC(Nc2cnn(C)c(=O)c2Cl)CC(c2ccc(C(=O)N3CCC4(CC3)CCN(c3ccc5c(c3)oc(=O)n5C3CCC(=O)NC3=O)CC4)cc2)C1] |
| 210 | 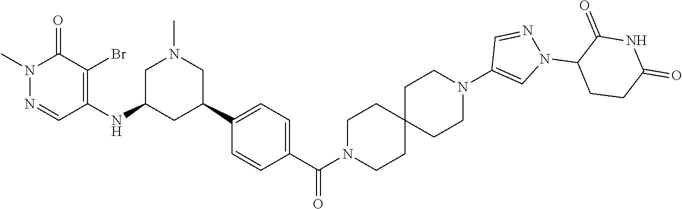 [SMILES: CN1C[C@H](Nc2cnn(C)c(=O)c2Br)C[C@H](c2ccc(C(=O)N3CCC4(CC3)CCN(c3cnn(C5CCC(=O)NC5=O)c3)CC4)cc2)C1] |
| 210a | 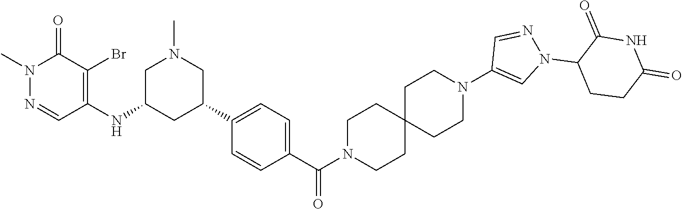 [SMILES: CN1C[C@@H](Nc2cnn(C)c(=O)c2Br)C[C@@H](c2ccc(C(=O)N3CCC4(CC3)CCN(c3cnn(C5CCC(=O)NC5=O)c3)CC4)cc2)C1] |
| 210b | 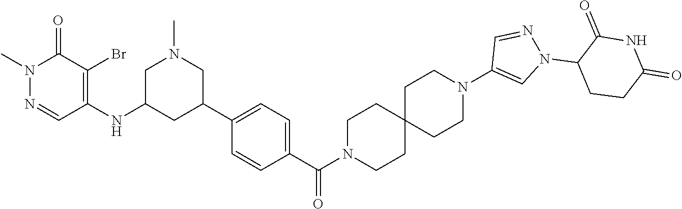 [SMILES: CN1CC(Nc2cnn(C)c(=O)c2Br)CC(c2ccc(C(=O)N3CCC4(CC3)CCN(c3cnn(C5CCC(=O)NC5=O)c3)CC4)cc2)C1] |
| 211 | 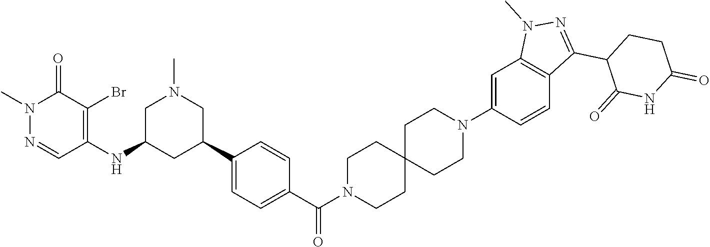 [SMILES: CN1C[C@H](Nc2cnn(C)c(=O)c2Br)C[C@H](c2ccc(C(=O)N3CCC4(CC3)CCN(c3ccc5c(C6CCC(=O)NC6=O)nn(C)c5c3)CC4)cc2)C1] |
| 211a | 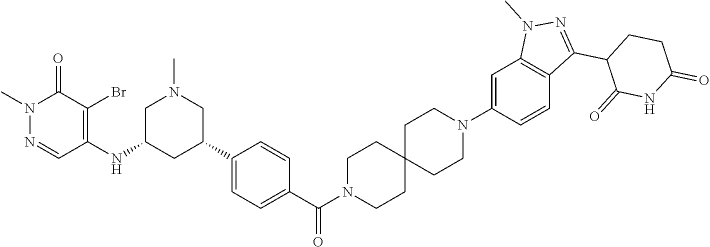 [SMILES: CN1C[C@@H](Nc2cnn(C)c(=O)c2Br)C[C@@H](c2ccc(C(=O)N3CCC4(CC3)CCN(c3ccc5c(C6CCC(=O)NC6=O)nn(C)c5c3)CC4)cc2)C1] |
| 211b | 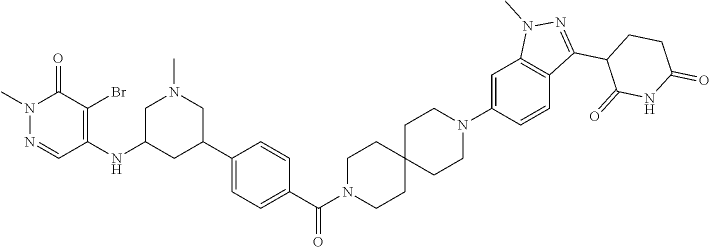 [SMILES: CN1CC(Nc2cnn(C)c(=O)c2Br)CC(c2ccc(C(=O)N3CCC4(CC3)CCN(c3ccc5c(C6CCC(=O)NC6=O)nn(C)c5c3)CC4)cc2)C1] |
| 212 | 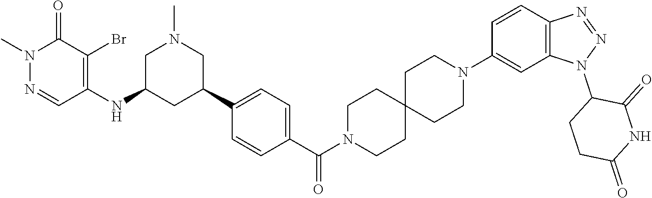 [SMILES: CN1C[C@H](Nc2cnn(C)c(=O)c2Br)C[C@H](c2ccc(C(=O)N3CCC4(CC3)CCN(c3ccc5nnn(C6CCC(=O)NC6=O)c5c3)CC4)cc2)C1] |
| 212a | 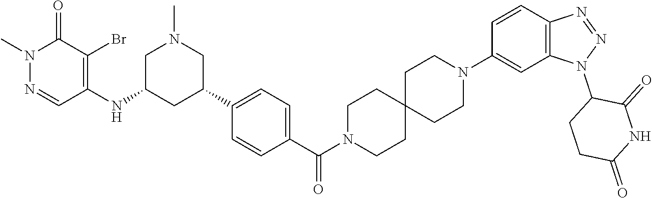 [SMILES: CN1C[C@@H](Nc2cnn(C)c(=O)c2Br)C[C@@H](c2ccc(C(=O)N3CCC4(CC3)CCN(c3ccc5nnn(C6CCC(=O)NC6=O)c5c3)CC4)cc2)C1] |
| 212b | 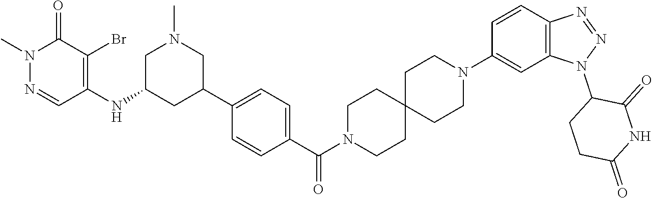 [SMILES: CN1CC(c2ccc(C(=O)N3CCC4(CC3)CCN(c3ccc5nnn(C6CCC(=O)NC6=O)c5c3)CC4)cc2)C[C@H](Nc2cnn(C)c(=O)c2Br)C1] |
| 213 | 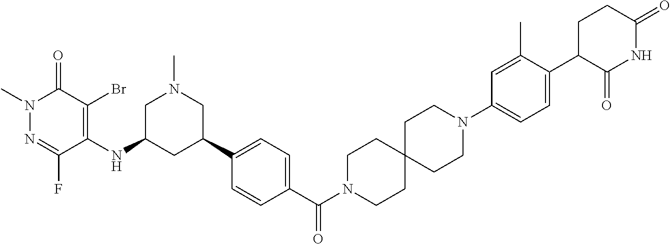 [SMILES: Cc1cc(N2CCC3(CCN(C(=O)c4ccc([C@H]5C[C@@H](Nc6c(F)nn(C)c(=O)c6Br)CN(C)C5)cc4)CC3)CC2)ccc1C1CCC(=O)NC1=O] |
| 213a | 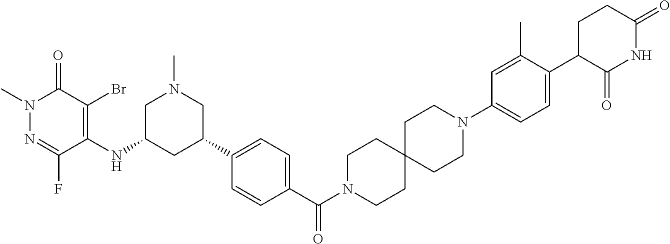 [SMILES: Cc1cc(N2CCC3(CCN(C(=O)c4ccc([C@@H]5C[C@H](Nc6c(F)nn(C)c(=O)c6Br)CN(C)C5)cc4)CC3)CC2)ccc1C1CCC(=O)NC1=O] |
| 213b | 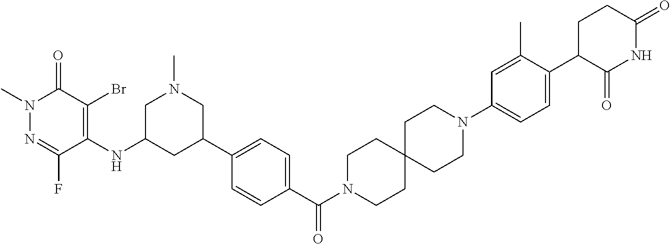 [SMILES: Cc1cc(N2CCC3(CCN(C(=O)c4ccc(C5CC(Nc6c(F)nn(C)c(=O)c6Br)CN(C)C5)cc4)CC3)CC2)ccc1C1CCC(=O)NC1=O] |
| 214 | 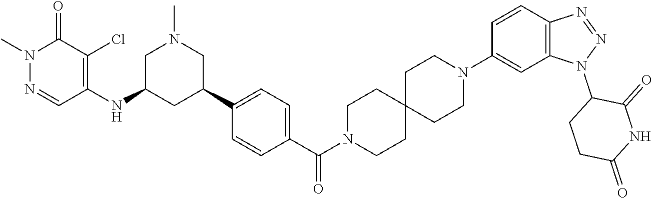 [SMILES: CN1C[C@H](Nc2cnn(C)c(=O)c2Cl)C[C@H](c2ccc(C(=O)N3CCC4(CC3)CCN(c3ccc5nnn(C6CCC(=O)NC6=O)c5c3)CC4)cc2)C1] |
| 214a | 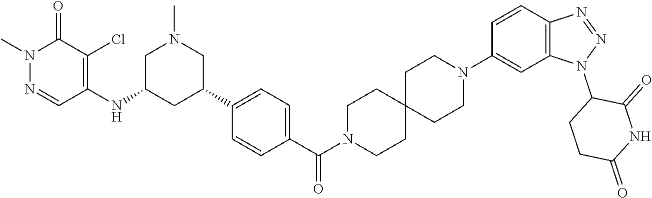 [SMILES: CN1C[C@@H](Nc2cnn(C)c(=O)c2Cl)C[C@@H](c2ccc(C(=O)N3CCC4(CC3)CCN(c3ccc5nnn(C6CCC(=O)NC6=O)c5c3)CC4)cc2)C1] |
| 214b | 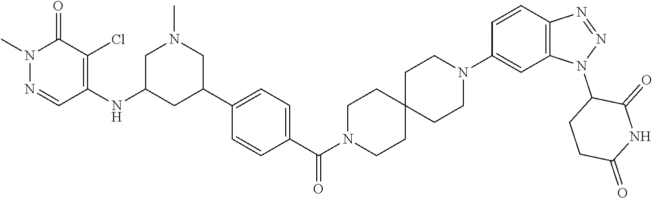 [SMILES: CN1CC(Nc2cnn(C)c(=O)c2Cl)CC(c2ccc(C(=O)N3CCC4(CC3)CCN(c3ccc5nnn(C6CCC(=O)NC6=O)c5c3)CC4)cc2)C1] |
| 215 | 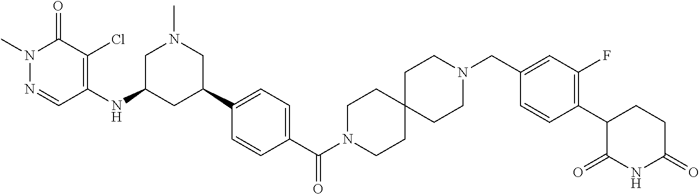 [SMILES: CN1C[C@H](Nc2cnn(C)c(=O)c2Cl)C[C@H](c2ccc(C(=O)N3CCC4(CCN(Cc5ccc(C6CCC(=O)NC6=O)c(F)c5)CC4)CC3)cc2)C1] |
| 215a | 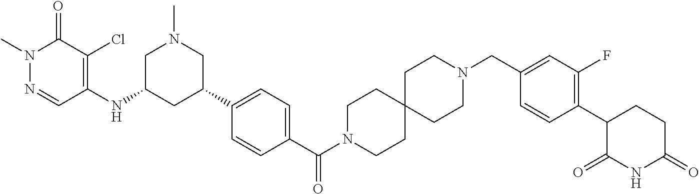 [SMILES: CN1C[C@@H](Nc2cnn(C)c(=O)c2Cl)C[C@@H](c2ccc(C(=O)N3CCC4(CCN(Cc5ccc(C6CCC(=O)NC6=O)c(F)c5)CC4)CC3)cc2)C1] |
| 215b | 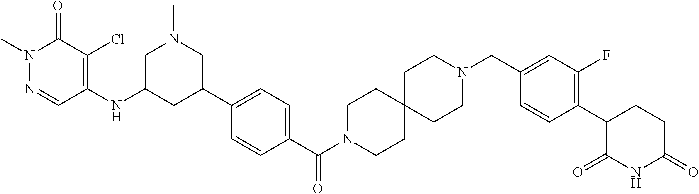 [SMILES: CN1CC(Nc2cnn(C)c(=O)c2Cl)CC(c2ccc(C(=O)N3CCC4(CCN(Cc5ccc(C6CCC(=O)NC6=O)c(F)c5)CC4)CC3)cc2)C1] |
| 216 | 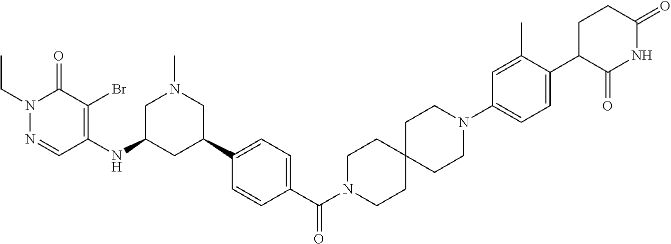 [SMILES: CCn1ncc(N[C@@H]2C[C@H](c3ccc(C(=O)N4CCC5(CC4)CCN(c4ccc(C6CCC(=O)NC6=O)c(C)c4)CC5)cc3)CN(C)C2)c(Br)c1=O] |
| 216a | 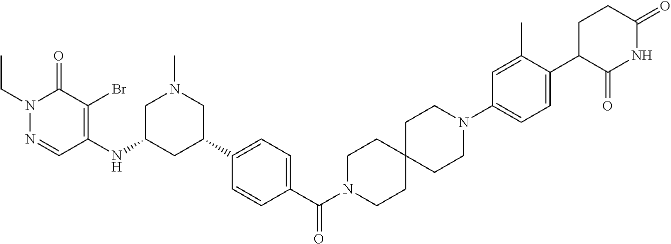 [SMILES: CCn1ncc(N[C@H]2C[C@@H](c3ccc(C(=O)N4CCC5(CC4)CCN(c4ccc(C6CCC(=O)NC6=O)c(C)c4)CC5)cc3)CN(C)C2)c(Br)c1=O] |
| 216b | 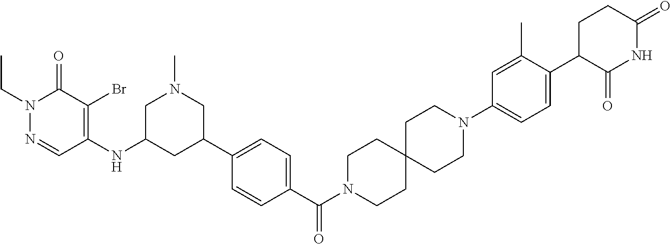 [SMILES: CCn1ncc(NC2CC(c3ccc(C(=O)N4CCC5(CC4)CCN(c4ccc(C6CCC(=O)NC6=O)c(C)c4)CC5)cc3)CN(C)C2)c(Br)c1=O] |
| 217 | 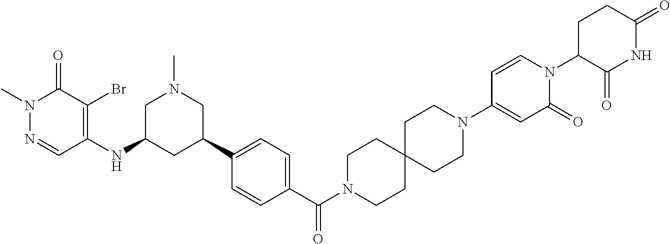 [SMILES: CN1C[C@H](Nc2cnn(C)c(=O)c2Br)C[C@H](c2ccc(C(=O)N3CCC4(CC3)CCN(c3ccn(C5CCC(=O)NC5=O)c(=O)c3)CC4)cc2)C1] |
| 217a | 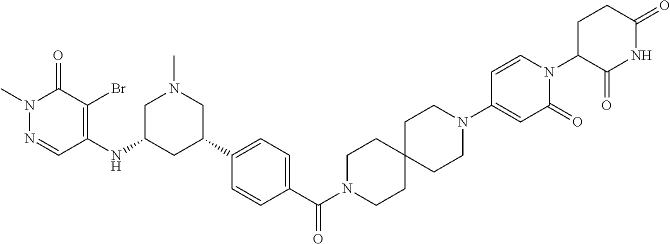 [SMILES: CN1C[C@@H](Nc2cnn(C)c(=O)c2Br)C[C@@H](c2ccc(C(=O)N3CCC4(CC3)CCN(c3ccn(C5CCC(=O)NC5=O)c(=O)c3)CC4)cc2)C1] |
| 217b | 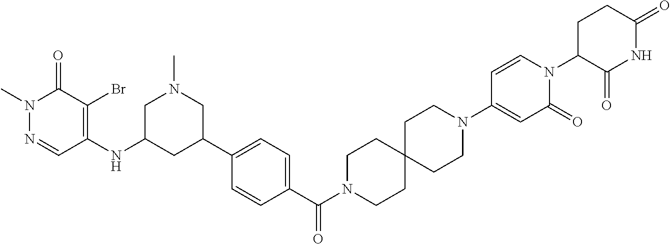 [SMILES: CN1CC(Nc2cnn(C)c(=O)c2Br)CC(c2ccc(C(=O)N3CCC4(CC3)CCN(c3ccn(C5CCC(=O)NC5=O)c(=O)c3)CC4)cc2)C1] |
| 218 | 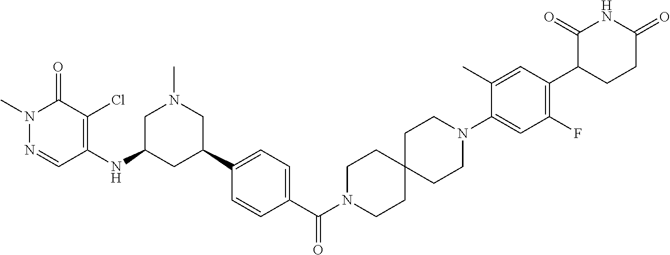 [SMILES: Cc1cc(C2CCC(=O)NC2=O)c(F)cc1N1CCC2(CCN(C(=O)c3ccc([C@H]4C[C@@H](Nc5cnn(C)c(=O)c5Cl)CN(C)C4)cc3)CC2)CC1] |
| 218a | 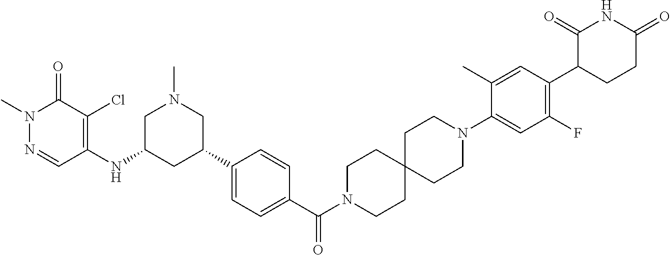 [SMILES: Cc1cc(C2CCC(=O)NC2=O)c(F)cc1N1CCC2(CCN(C(=O)c3ccc([C@@H]4C[C@H](Nc5cnn(C)c(=O)c5Cl)CN(C)C4)cc3)CC2)CC1] |
| 218b | 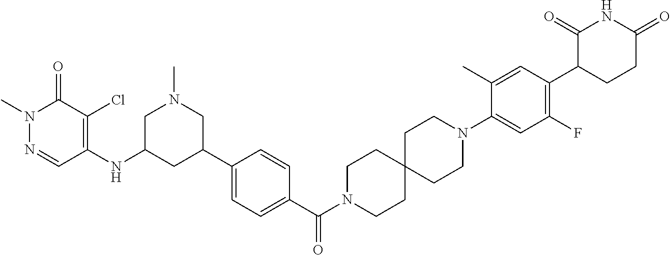 [SMILES: Cc1cc(C2CCC(=O)NC2=O)c(F)cc1N1CCC2(CCN(C(=O)c3ccc(C4CC(Nc5cnn(C)c(=O)c5Cl)CN(C)C4)cc3)CC2)CC1] |
| 219 | 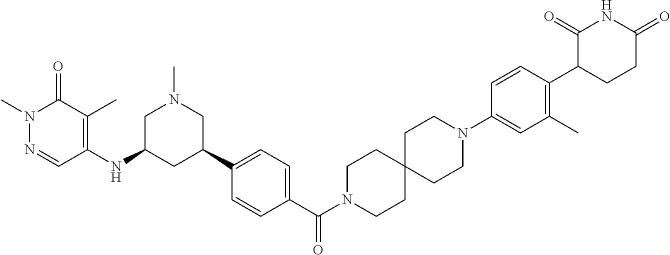 [SMILES: Cc1cc(N2CCC3(CCN(C(=O)c4ccc([C@H]5C[C@@H](Nc6cnn(C)c(=O)c6C)CN(C)C5)cc4)CC3)CC2)ccc1C1CCC(=O)NC1=O] |
| 219a | 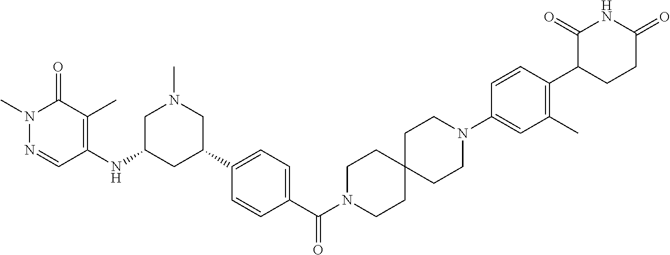 [SMILES: Cc1cc(N2CCC3(CCN(C(=O)c4ccc([C@@H]5C[C@H](Nc6cnn(C)c(=O)c6C)CN(C)C5)cc4)CC3)CC2)ccc1C1CCC(=O)NC1=O] |
| 219b | 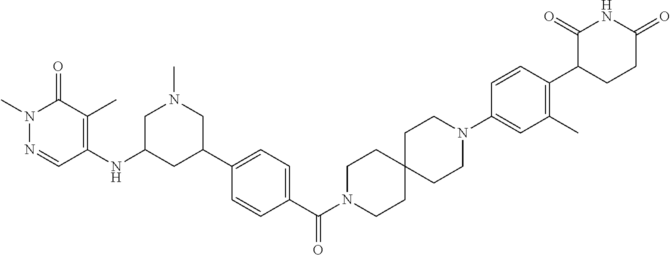 [SMILES: Cc1cc(N2CCC3(CCN(C(=O)c4ccc(C5CC(Nc6cnn(C)c(=O)c6C)CN(C)C5)cc4)CC3)CC2)ccc1C1CCC(=O)NC1=O] |
| 220 | 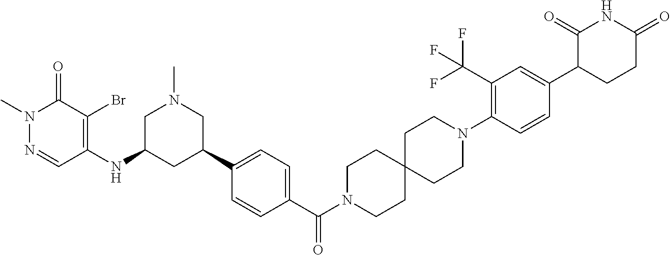 [SMILES: CN1C[C@H](Nc2cnn(C)c(=O)c2Br)C[C@H](c2ccc(C(=O)N3CCC4(CC3)CCN(c3ccc(C5CCC(=O)NC5=O)cc3C(F)(F)F)CC4)cc2)C1] |
| 220a | 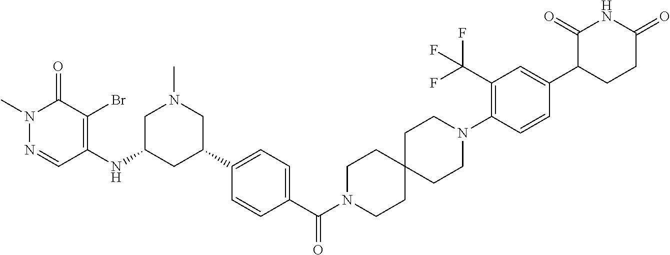 [SMILES: CN1C[C@@H](Nc2cnn(C)c(=O)c2Br)C[C@@H](c2ccc(C(=O)N3CCC4(CC3)CCN(c3ccc(C5CCC(=O)NC5=O)cc3C(F)(F)F)CC4)cc2)C1] |
| 220b | 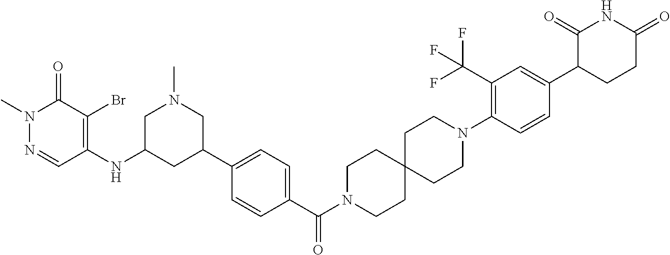 [SMILES: CN1CC(Nc2cnn(C)c(=O)c2Br)CC(c2ccc(C(=O)N3CCC4(CC3)CCN(c3ccc(C5CCC(=O)NC5=O)cc3C(F)(F)F)CC4)cc2)C1] |
| 221 | 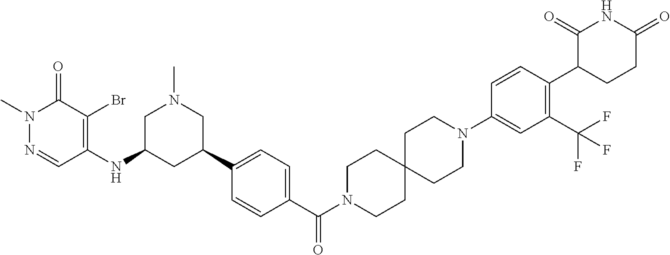 [SMILES: CN1C[C@H](Nc2cnn(C)c(=O)c2Br)C[C@H](c2ccc(C(=O)N3CCC4(CC3)CCN(c3ccc(C5CCC(=O)NC5=O)c(C(F)(F)F)c3)CC4)cc2)C1] |
| 221a | 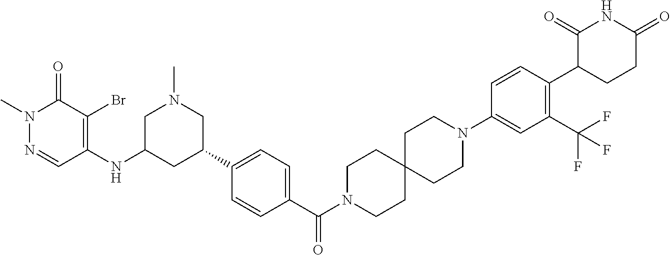 [SMILES: CN1CC(Nc2cnn(C)c(=O)c2Br)C[C@@H](c2ccc(C(=O)N3CCC4(CC3)CCN(c3ccc(C5CCC(=O)NC5=O)c(C(F)(F)F)c3)CC4)cc2)C1] |
| 221b | 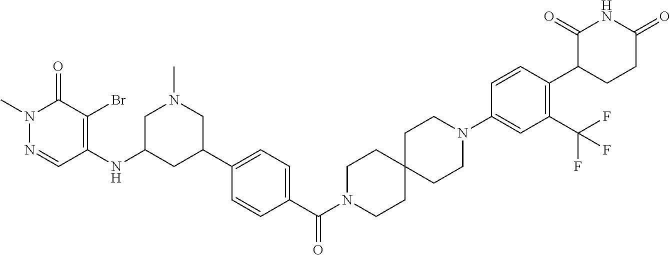 [SMILES: CN1CC(Nc2cnn(C)c(=O)c2Br)CC(c2ccc(C(=O)N3CCC4(CC3)CCN(c3ccc(C5CCC(=O)NC5=O)c(C(F)(F)F)c3)CC4)cc2)C1] |
| 222 | 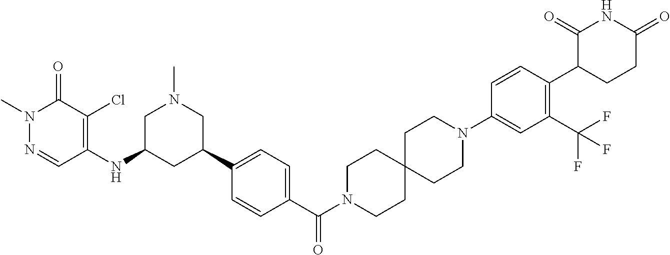 [SMILES: CN1C[C@H](Nc2cnn(C)c(=O)c2Cl)C[C@H](c2ccc(C(=O)N3CCC4(CC3)CCN(c3ccc(C5CCC(=O)NC5=O)c(C(F)(F)F)c3)CC4)cc2)C1] |
| 222a | 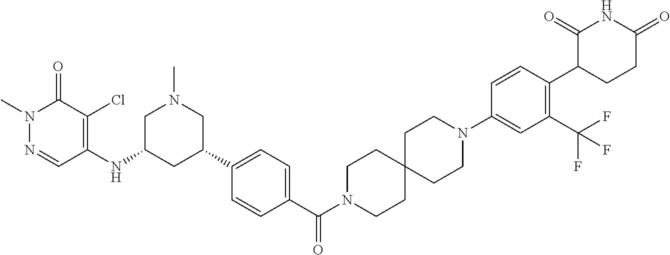 [SMILES: CN1C[C@@H](Nc2cnn(C)c(=O)c2Cl)C[C@@H](c2ccc(C(=O)N3CCC4(CC3)CCN(c3ccc(C5CCC(=O)NC5=O)c(C(F)(F)F)c3)CC4)cc2)C1] |
| 222b | 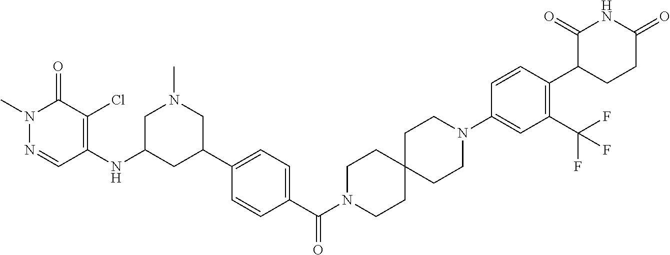 [SMILES: CN1CC(Nc2cnn(C)c(=O)c2Cl)CC(c2ccc(C(=O)N3CCC4(CC3)CCN(c3ccc(C5CCC(=O)NC5=O)c(C(F)(F)F)c3)CC4)cc2)C1] |
| 223 | 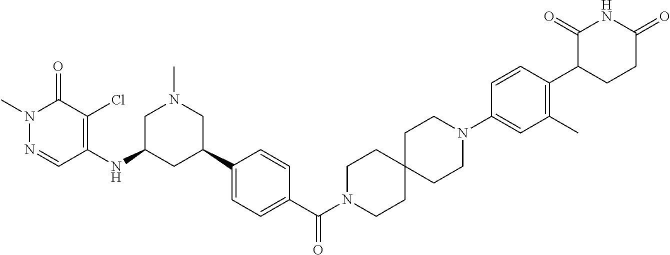 [SMILES: Cc1cc(N2CCC3(CCN(C(=O)c4ccc([C@H]5C[C@@H](Nc6cnn(C)c(=O)c6Cl)CN(C)C5)cc4)CC3)CC2)ccc1C1CCC(=O)NC1=O] |
| 223a | 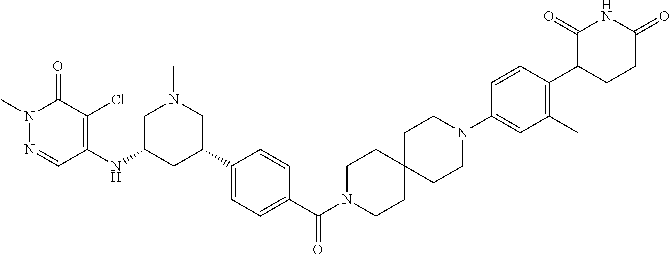 [SMILES: Cc1cc(N2CCC3(CCN(C(=O)c4ccc([C@@H]5C[C@H](Nc6cnn(C)c(=O)c6Cl)CN(C)C5)cc4)CC3)CC2)ccc1C1CCC(=O)NC1=O] |
| 223b | 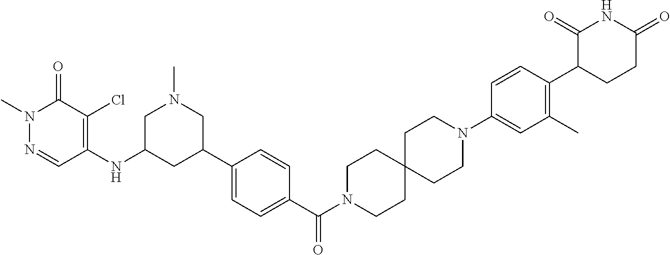 [SMILES: Cc1cc(N2CCC3(CCN(C(=O)c4ccc(C5CC(Nc6cnn(C)c(=O)c6Cl)CN(C)C5)cc4)CC3)CC2)ccc1C1CCC(=O)NC1=O] |
| 224 | 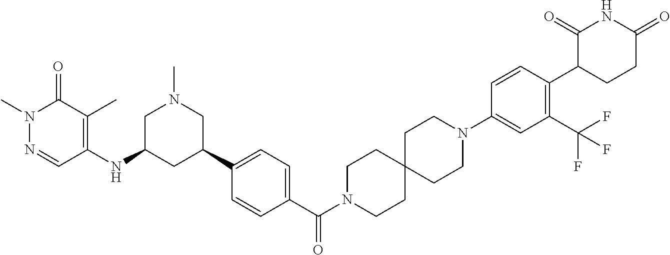 [SMILES: Cc1c(N[C@@H]2C[C@H](c3ccc(C(=O)N4CCC5(CC4)CCN(c4ccc(C6CCC(=O)NC6=O)c(C(F)(F)F)c4)CC5)cc3)CN(C)C2)cnn(C)c1=O] |
| 224a | 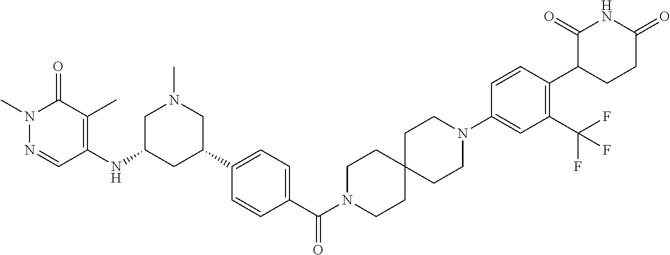 [SMILES: Cc1c(N[C@H]2C[C@@H](c3ccc(C(=O)N4CCC5(CC4)CCN(c4ccc(C6CCC(=O)NC6=O)c(C(F)(F)F)c4)CC5)cc3)CN(C)C2)cnn(C)c1=O] |
| 224b | 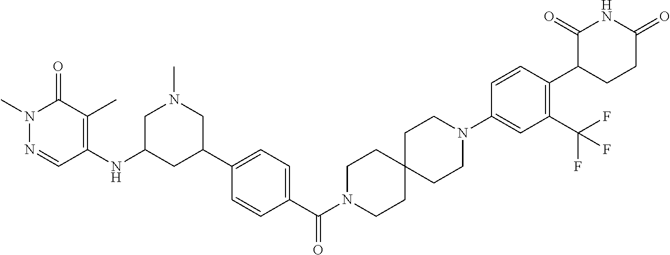 [SMILES: Cc1c(NC2CC(c3ccc(C(=O)N4CCC5(CC4)CCN(c4ccc(C6CCC(=O)NC6=O)c(C(F)(F)F)c4)CC5)cc3)CN(C)C2)cnn(C)c1=O] |
| 225 | 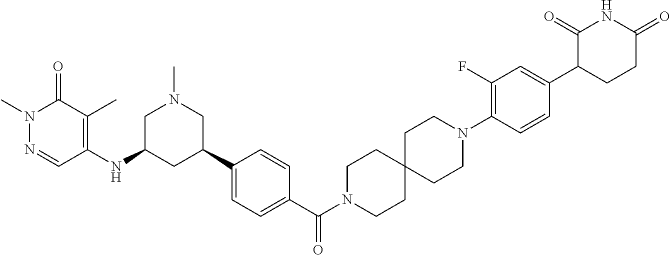 [SMILES: Cc1c(N[C@@H]2C[C@H](c3ccc(C(=O)N4CCC5(CC4)CCN(c4ccc(C6CCC(=O)NC6=O)cc4F)CC5)cc3)CN(C)C2)cnn(C)c1=O] |
| 225a | 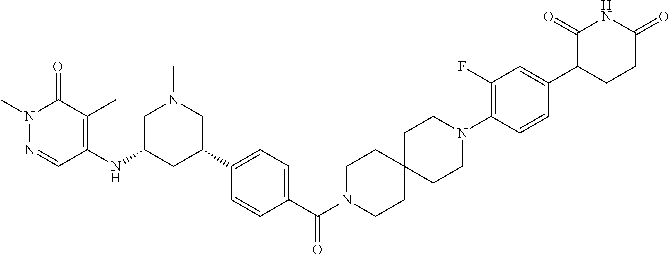 [SMILES: Cc1c(N[C@H]2C[C@@H](c3ccc(C(=O)N4CCC5(CC4)CCN(c4ccc(C6CCC(=O)NC6=O)cc4F)CC5)cc3)CN(C)C2)cnn(C)c1=O] |
| 225b | 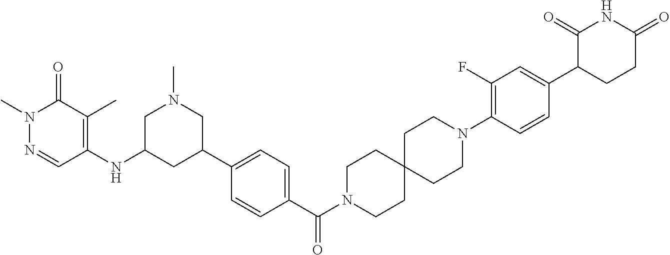 [SMILES: Cc1c(NC2CC(c3ccc(C(=O)N4CCC5(CC4)CCN(c4ccc(C6CCC(=O)NC6=O)cc4F)CC5)cc3)CN(C)C2)cnn(C)c1=O] |
| 226 | 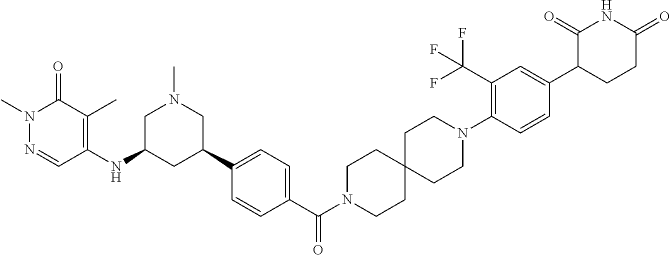 [SMILES: Cc1c(N[C@@H]2C[C@H](c3ccc(C(=O)N4CCC5(CC4)CCN(c4ccc(C6CCC(=O)NC6=O)cc4C(F)(F)F)CC5)cc3)CN(C)C2)cnn(C)c1=O] |
| 226a | 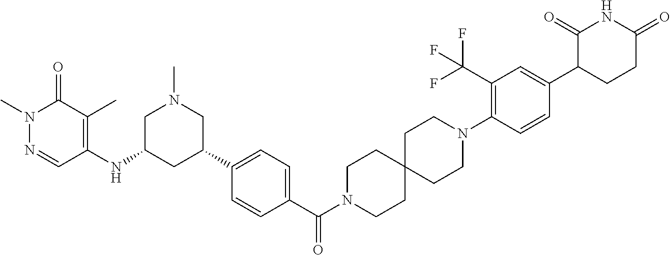 [SMILES: Cc1c(N[C@H]2C[C@@H](c3ccc(C(=O)N4CCC5(CC4)CCN(c4ccc(C6CCC(=O)NC6=O)cc4C(F)(F)F)CC5)cc3)CN(C)C2)cnn(C)c1=O] |
| 226b | 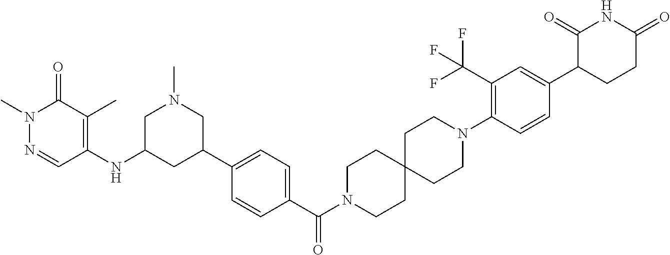 [SMILES: Cc1c(NC2CC(c3ccc(C(=O)N4CCC5(CC4)CCN(c4ccc(C6CCC(=O)NC6=O)cc4C(F)(F)F)CC5)cc3)CN(C)C2)cnn(C)c1=O] |
| 227 | 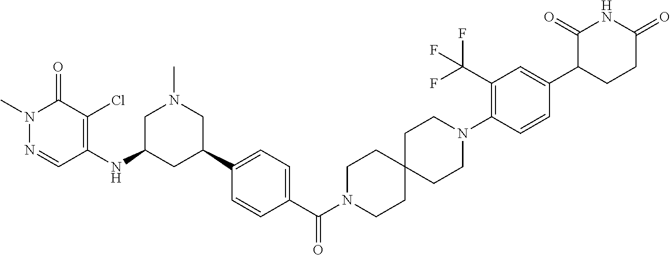 [SMILES: CN1C[C@H](Nc2cnn(C)c(=O)c2Cl)C[C@H](c2ccc(C(=O)N3CCC4(CC3)CCN(c3ccc(C5CCC(=O)NC5=O)cc3C(F)(F)F)CC4)cc2)C1] |
| 227a | 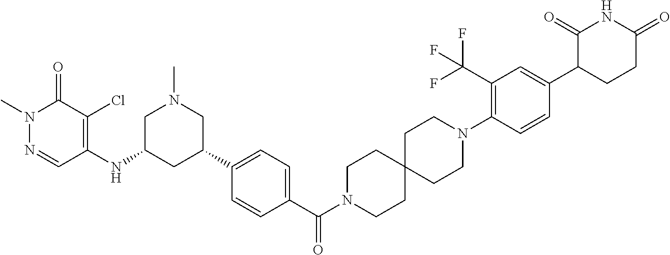 [SMILES: CN1C[C@@H](Nc2cnn(C)c(=O)c2Cl)C[C@@H](c2ccc(C(=O)N3CCC4(CC3)CCN(c3ccc(C5CCC(=O)NC5=O)cc3C(F)(F)F)CC4)cc2)C1] |
| 227b | 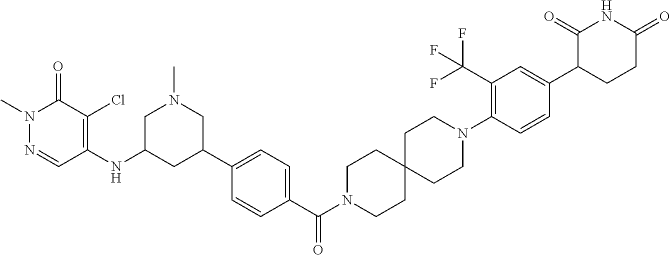 [SMILES: CN1CC(Nc2cnn(C)c(=O)c2Cl)CC(c2ccc(C(=O)N3CCC4(CC3)CCN(c3ccc(C5CCC(=O)NC5=O)cc3C(F)(F)F)CC4)cc2)C1] |
| 228 | 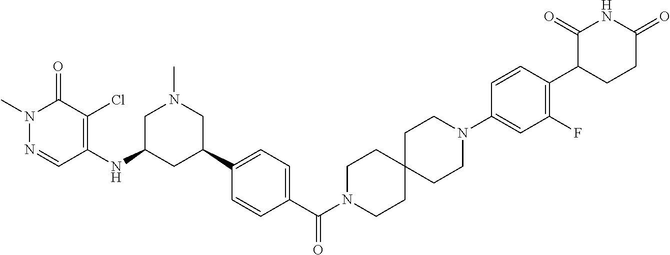 [SMILES: CN1C[C@H](Nc2cnn(C)c(=O)c2Cl)C[C@H](c2ccc(C(=O)N3CCC4(CC3)CCN(c3ccc(C5CCC(=O)NC5=O)c(F)c3)CC4)cc2)C1] |
| 228-1 | 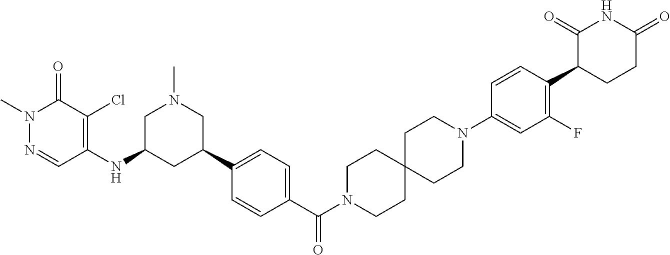 [SMILES: CN1C[C@H](Nc2cnn(C)c(=O)c2Cl)C[C@H](c2ccc(C(=O)N3CCC4(CC3)CCN(c3ccc([C@@H]5CCC(=O)NC5=O)c(F)c3)CC4)cc2)C1] |
| 228-2 | 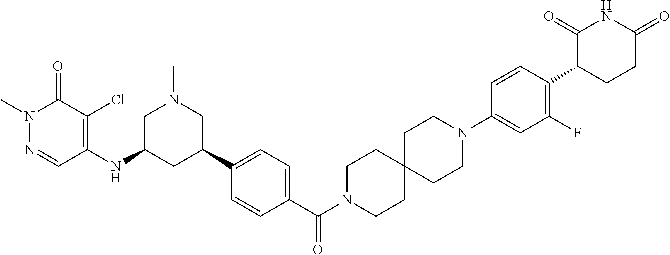 [SMILES: CN1C[C@H](Nc2cnn(C)c(=O)c2Cl)C[C@H](c2ccc(C(=O)N3CCC4(CC3)CCN(c3ccc([C@H]5CCC(=O)NC5=O)c(F)c3)CC4)cc2)C1] |
| 228a | 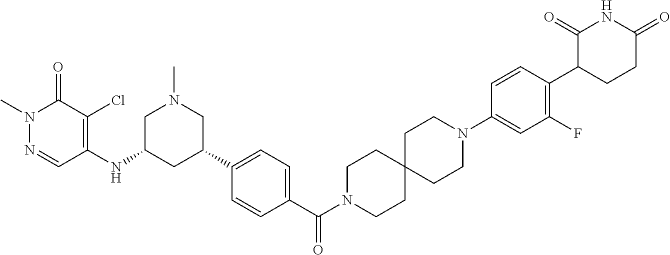 [SMILES: CN1C[C@@H](Nc2cnn(C)c(=O)c2Cl)C[C@@H](c2ccc(C(=O)N3CCC4(CC3)CCN(c3ccc(C5CCC(=O)NC5=O)c(F)c3)CC4)cc2)C1] |
| 228b | 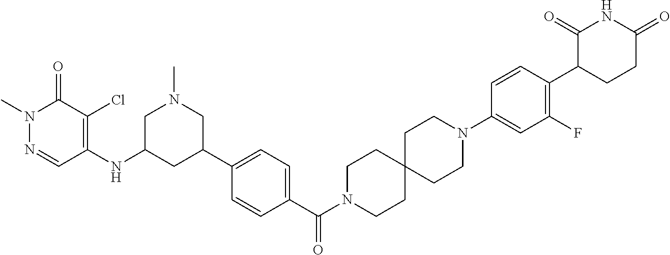 [SMILES: CN1CC(Nc2cnn(C)c(=O)c2Cl)CC(c2ccc(C(=O)N3CCC4(CC3)CCN(c3ccc(C5CCC(=O)NC5=O)c(F)c3)CC4)cc2)C1] |
| 229 | 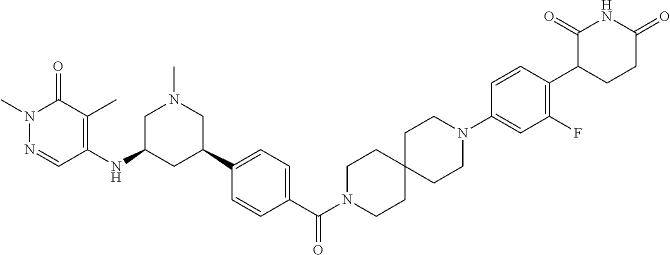 [SMILES: Cc1c(N[C@@H]2C[C@H](c3ccc(C(=O)N4CCC5(CC4)CCN(c4ccc(C6CCC(=O)NC6=O)c(F)c4)CC5)cc3)CN(C)C2)cnn(C)c1=O] |
| 229-1 | 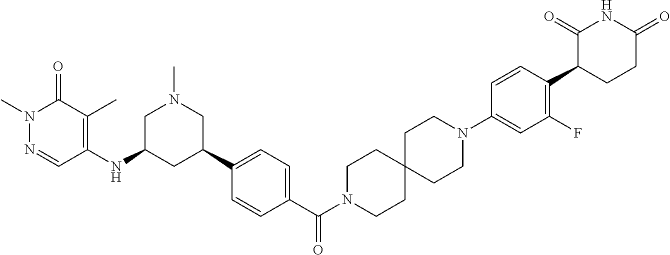 [SMILES: Cc1c(N[C@@H]2C[C@H](c3ccc(C(=O)N4CCC5(CC4)CCN(c4ccc([C@@H]6CCC(=O)NC6=O)c(F)c4)CC5)cc3)CN(C)C2)cnn(C)c1=O] |
| 229-2 | 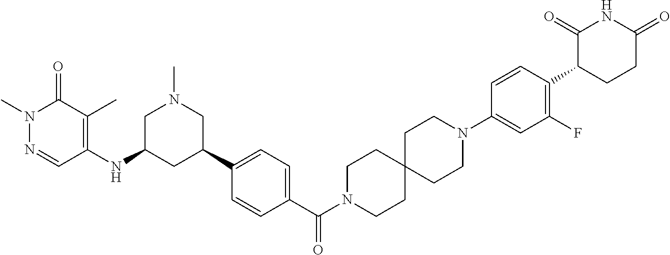 [SMILES: Cc1c(N[C@@H]2C[C@H](c3ccc(C(=O)N4CCC5(CC4)CCN(c4ccc([C@H]6CCC(=O)NC6=O)c(F)c4)CC5)cc3)CN(C)C2)cnn(C)c1=O] |
| 229a | 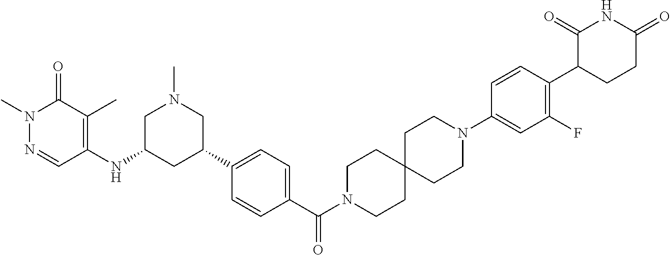 [SMILES: Cc1c(N[C@H]2C[C@@H](c3ccc(C(=O)N4CCC5(CC4)CCN(c4ccc(C6CCC(=O)NC6=O)c(F)c4)CC5)cc3)CN(C)C2)cnn(C)c1=O] |
| 229b | 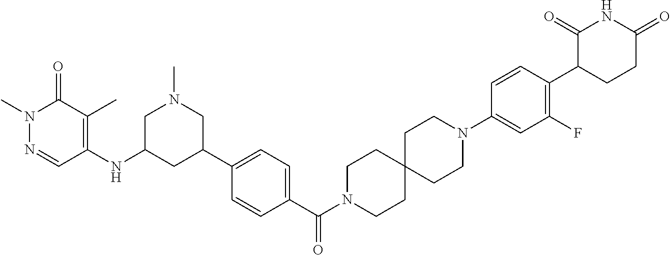 [SMILES: Cc1c(NC2CC(c3ccc(C(=O)N4CCC5(CC4)CCN(c4ccc(C6CCC(=O)NC6=O)c(F)c4)CC5)cc3)CN(C)C2)cnn(C)c1=O] |
| 230 | 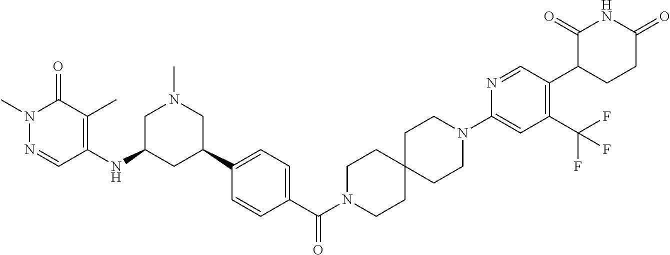 [SMILES: Cc1c(N[C@@H]2C[C@H](c3ccc(C(=O)N4CCC5(CC4)CCN(c4cc(C(F)(F)F)c(C6CCC(=O)NC6=O)cn4)CC5)cc3)CN(C)C2)cnn(C)c1=O] |
| 230a | 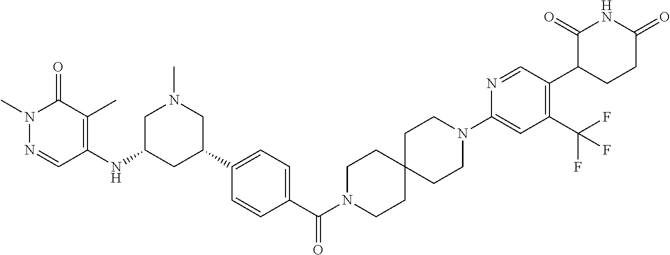 [SMILES: Cc1c(N[C@H]2C[C@@H](c3ccc(C(=O)N4CCC5(CC4)CCN(c4cc(C(F)(F)F)c(C6CCC(=O)NC6=O)cn4)CC5)cc3)CN(C)C2)cnn(C)c1=O] |
| 230b | 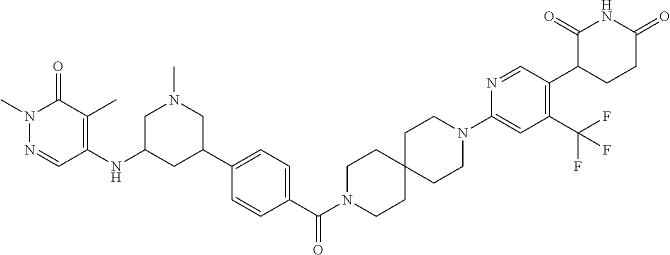 [SMILES: Cc1c(NC2CC(c3ccc(C(=O)N4CCC5(CC4)CCN(c4cc(C(F)(F)F)c(C6CCC(=O)NC6=O)cn4)CC5)cc3)CN(C)C2)cnn(C)c1=O] |
| 231 | 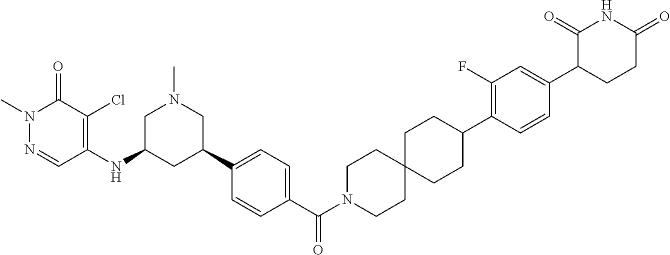 [SMILES: CN1C[C@H](Nc2cnn(C)c(=O)c2Cl)C[C@H](c2ccc(C(=O)N3CCC4(CCC(c5ccc(C6CCC(=O)NC6=O)cc5F)CC4)CC3)cc2)C1] |
| 231a | 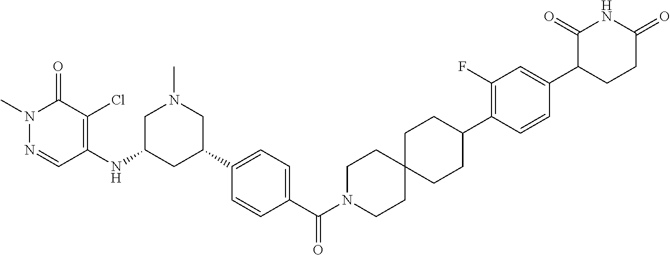 [SMILES: CN1C[C@@H](Nc2cnn(C)c(=O)c2Cl)C[C@@H](c2ccc(C(=O)N3CCC4(CCC(c5ccc(C6CCC(=O)NC6=O)cc5F)CC4)CC3)cc2)C1] |
| 231b | 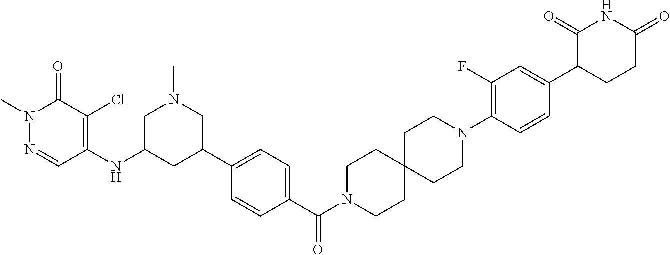 [SMILES: CN1CC(Nc2cnn(C)c(=O)c2Cl)CC(c2ccc(C(=O)N3CCC4(CC3)CCN(c3ccc(C5CCC(=O)NC5=O)cc3F)CC4)cc2)C1] |
| 232 | 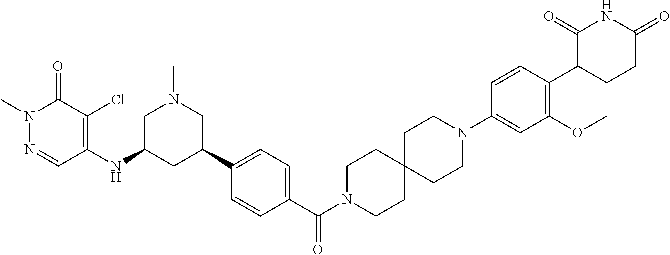 [SMILES: COc1cc(N2CCC3(CCN(C(=O)c4ccc([C@H]5C[C@@H](Nc6cnn(C)c(=O)c6Cl)CN(C)C5)cc4)CC3)CC2)ccc1C1CCC(=O)NC1=O] |
| 232a | 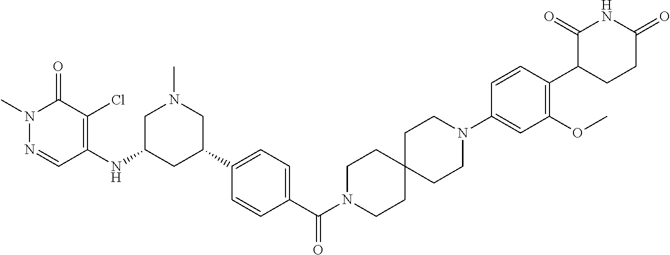 [SMILES: COc1cc(N2CCC3(CCN(C(=O)c4ccc([C@@H]5C[C@H](Nc6cnn(C)c(=O)c6Cl)CN(C)C5)cc4)CC3)CC2)ccc1C1CCC(=O)NC1=O] |
| 232b | 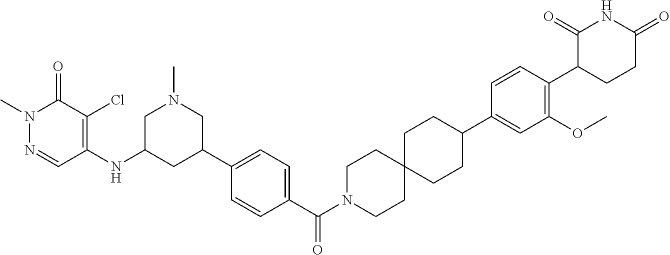 [SMILES: COc1cc(C2CCC3(CC2)CCN(C(=O)c2ccc(C4CC(Nc5cnn(C)c(=O)c5Cl)CN(C)C4)cc2)CC3)ccc1C1CCC(=O)NC1=O] |
| 233 | 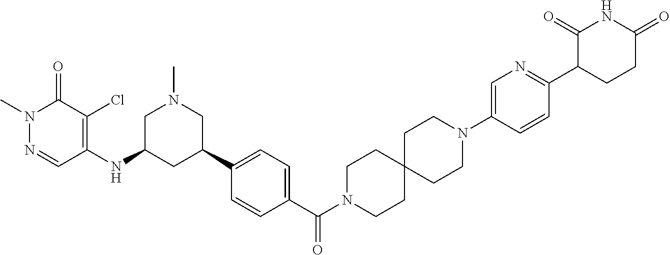 [SMILES: CN1C[C@H](Nc2cnn(C)c(=O)c2Cl)C[C@H](c2ccc(C(=O)N3CCC4(CC3)CCN(c3ccc(C5CCC(=O)NC5=O)nc3)CC4)cc2)C1] |
| 233a | 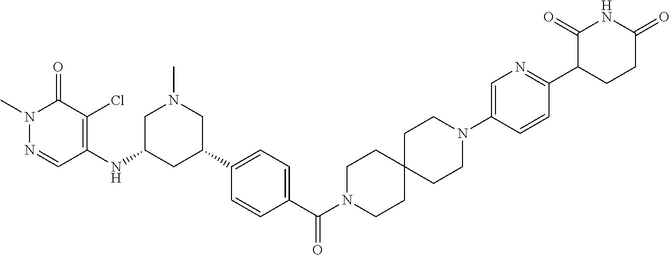 [SMILES: CN1C[C@@H](Nc2cnn(C)c(=O)c2Cl)C[C@@H](c2ccc(C(=O)N3CCC4(CC3)CCN(c3ccc(C5CCC(=O)NC5=O)nc3)CC4)cc2)C1] |
| 233b | 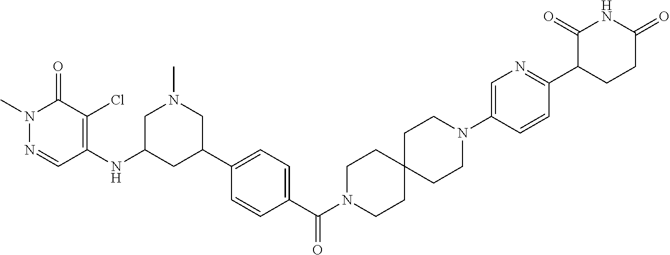 [SMILES: CN1CC(Nc2cnn(C)c(=O)c2Cl)CC(c2ccc(C(=O)N3CCC4(CC3)CCN(c3ccc(C5CCC(=O)NC5=O)nc3)CC4)cc2)C1] |
| 234 | 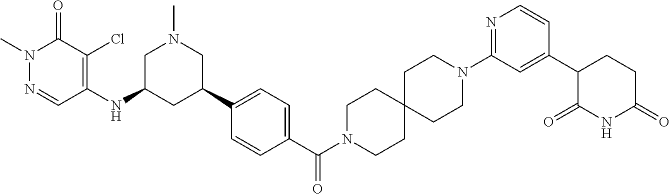 [SMILES: CN1C[C@H](Nc2cnn(C)c(=O)c2Cl)C[C@H](c2ccc(C(=O)N3CCC4(CC3)CCN(c3cc(C5CCC(=O)NC5=O)ccn3)CC4)cc2)C1] |
| 234a | 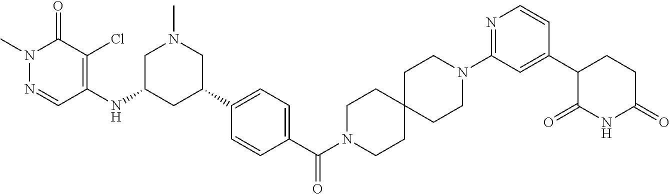 [SMILES: CN1C[C@@H](Nc2cnn(C)c(=O)c2Cl)C[C@@H](c2ccc(C(=O)N3CCC4(CC3)CCN(c3cc(C5CCC(=O)NC5=O)ccn3)CC4)cc2)C1] |
| 234b | 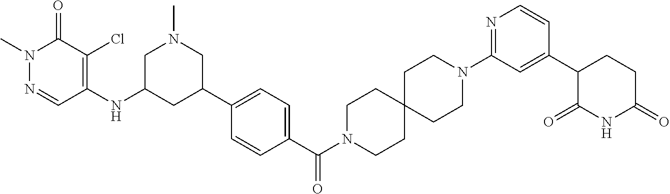 [SMILES: CN1CC(Nc2cnn(C)c(=O)c2Cl)CC(c2ccc(C(=O)N3CCC4(CC3)CCN(c3cc(C5CCC(=O)NC5=O)ccn3)CC4)cc2)C1] |
| 235 | 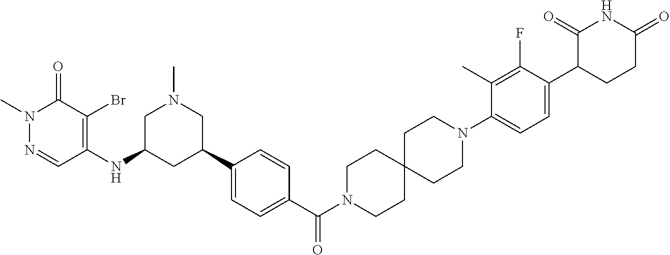 [SMILES: Cc1c(N2CCC3(CCN(C(=O)c4ccc([C@H]5C[C@@H](Nc6cnn(C)c(=O)c6Br)CN(C)C5)cc4)CC3)CC2)ccc(C2CCC(=O)NC2=O)c1F] |
| 235a | 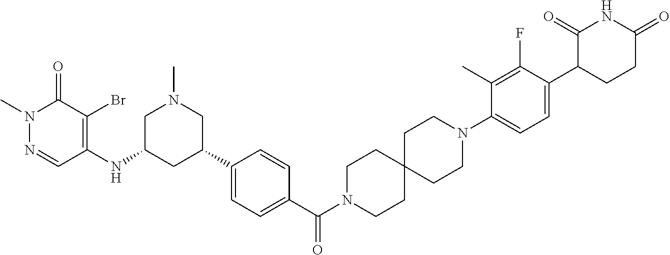 [SMILES: Cc1c(N2CCC3(CCN(C(=O)c4ccc([C@@H]5C[C@H](Nc6cnn(C)c(=O)c6Br)CN(C)C5)cc4)CC3)CC2)ccc(C2CCC(=O)NC2=O)c1F] |
| 235b | 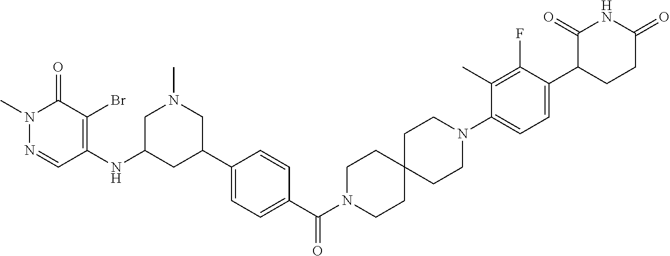 [SMILES: Cc1c(N2CCC3(CCN(C(=O)c4ccc(C5CC(Nc6cnn(C)c(=O)c6Br)CN(C)C5)cc4)CC3)CC2)ccc(C2CCC(=O)NC2=O)c1F] |
| 236 | 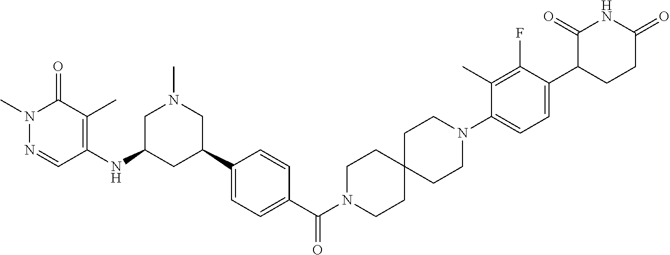 [SMILES: Cc1c(N2CCC3(CCN(C(=O)c4ccc([C@H]5C[C@@H](Nc6cnn(C)c(=O)c6C)CN(C)C5)cc4)CC3)CC2)ccc(C2CCC(=O)NC2=O)c1F] |
| 236a | 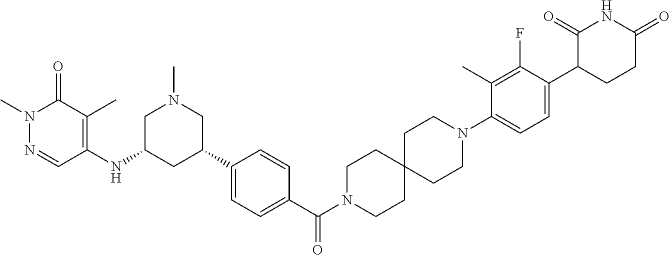 [SMILES: Cc1c(N2CCC3(CCN(C(=O)c4ccc([C@@H]5C[C@H](Nc6cnn(C)c(=O)c6C)CN(C)C5)cc4)CC3)CC2)ccc(C2CCC(=O)NC2=O)c1F] |
| 236b | 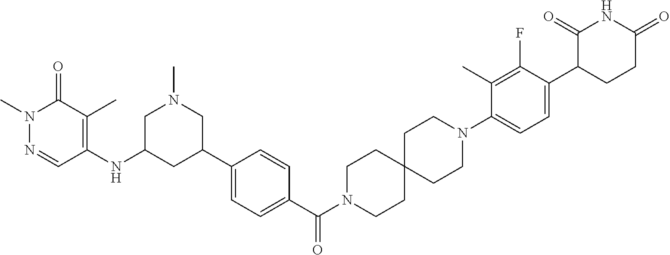 [SMILES: Cc1c(N2CCC3(CCN(C(=O)c4ccc(C5CC(Nc6cnn(C)c(=O)c6C)CN(C)C5)cc4)CC3)CC2)ccc(C2CCC(=O)NC2=O)c1F] |
| 237 | 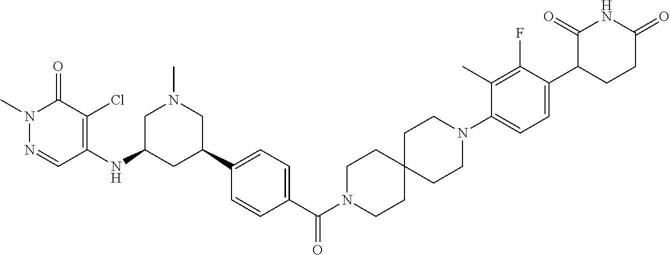 [SMILES: Cc1c(N2CCC3(CCN(C(=O)c4ccc([C@H]5C[C@@H](Nc6cnn(C)c(=O)c6Cl)CN(C)C5)cc4)CC3)CC2)ccc(C2CCC(=O)NC2=O)c1F] |
| 237a | 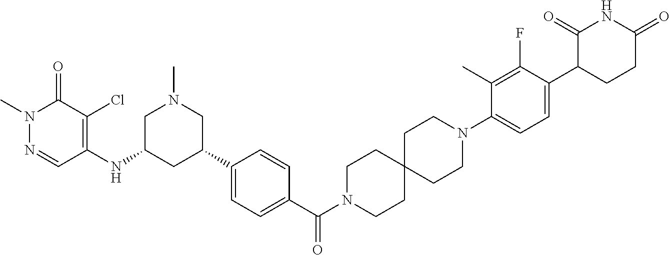 [SMILES: Cc1c(N2CCC3(CCN(C(=O)c4ccc([C@@H]5C[C@H](Nc6cnn(C)c(=O)c6Cl)CN(C)C5)cc4)CC3)CC2)ccc(C2CCC(=O)NC2=O)c1F] |
| 237b | 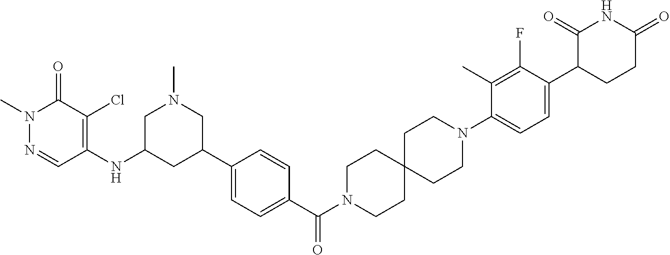 [SMILES: Cc1c(N2CCC3(CCN(C(=O)c4ccc(C5CC(Nc6cnn(C)c(=O)c6Cl)CN(C)C5)cc4)CC3)CC2)ccc(C2CCC(=O)NC2=O)c1F] |
| 238 | 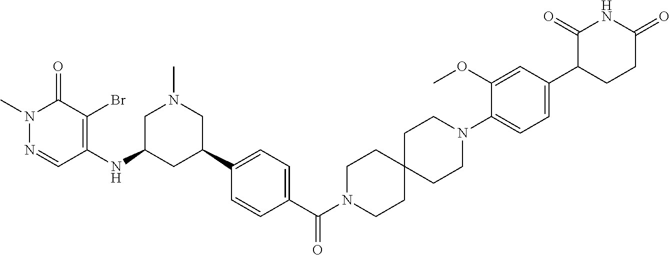 [SMILES: COc1cc(C2CCC(=O)NC2=O)ccc1N1CCC2(CCN(C(=O)c3ccc([C@H]4C[C@@H](Nc5cnn(C)c(=O)c5Br)CN(C)C4)cc3)CC2)CC1] |
| 238a | 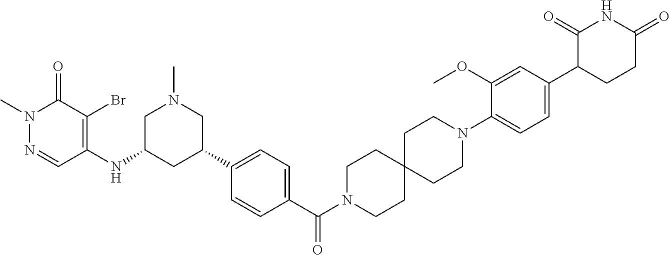 [SMILES: COc1cc(C2CCC(=O)NC2=O)ccc1N1CCC2(CCN(C(=O)c3ccc([C@@H]4C[C@H](Nc5cnn(C)c(=O)c5Br)CN(C)C4)cc3)CC2)CC1] |
| 238b | 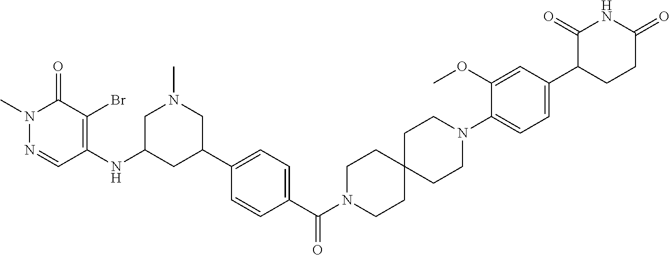 [SMILES: COc1cc(C2CCC(=O)NC2=O)ccc1N1CCC2(CCN(C(=O)c3ccc(C4CC(Nc5cnn(C)c(=O)c5Br)CN(C)C4)cc3)CC2)CC1] |
| 239/240-1 | 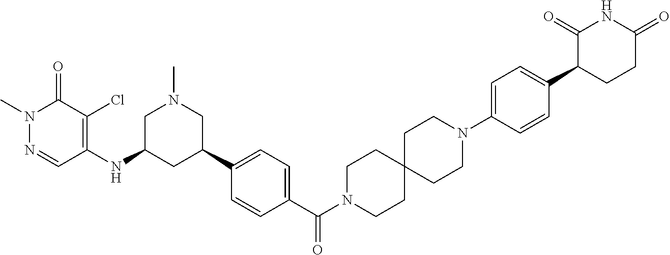 [SMILES: CN1C[C@H](Nc2cnn(C)c(=O)c2Cl)C[C@H](c2ccc(C(=O)N3CCC4(CC3)CCN(c3ccc([C@@H]5CCC(=O)NC5=O)cc3)CC4)cc2)C1] |
| 239/240-1a | 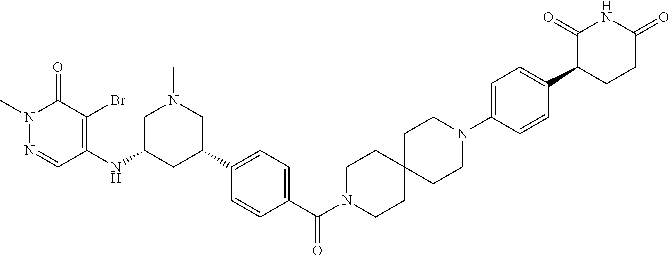 [SMILES: CN1C[C@@H](Nc2cnn(C)c(=O)c2Br)C[C@@H](c2ccc(C(=O)N3CCC4(CC3)CCN(c3ccc([C@@H]5CCC(=O)NC5=O)cc3)CC4)cc2)C1] |
| 239/240-1b | 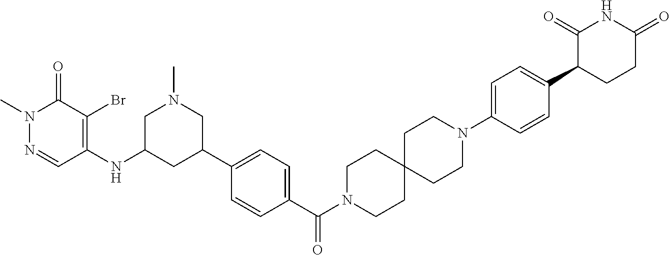 [SMILES: CN1CC(Nc2cnn(C)c(=O)c2Br)CC(c2ccc(C(=O)N3CCC4(CC3)CCN(c3ccc([C@@H]5CCC(=O)NC5=O)cc3)CC4)cc2)C1] |
| 239/240-2 | 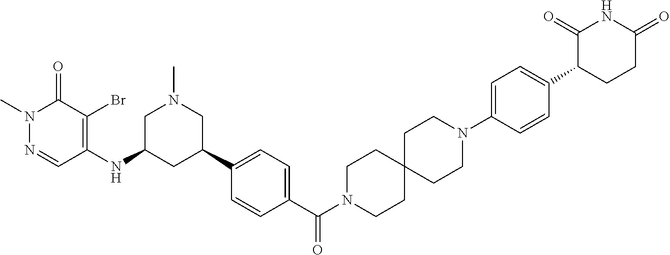 [SMILES: CN1C[C@H](Nc2cnn(C)c(=O)c2Br)C[C@H](c2ccc(C(=O)N3CCC4(CC3)CCN(c3ccc([C@H]5CCC(=O)NC5=O)cc3)CC4)cc2)C1] |
| 239/240-2a | 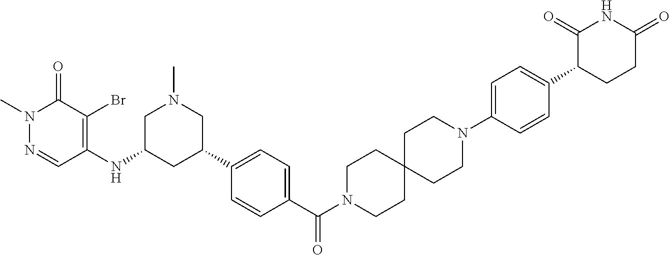 [SMILES: CN1C[C@@H](Nc2cnn(C)c(=O)c2Br)C[C@@H](c2ccc(C(=O)N3CCC4(CC3)CCN(c3ccc([C@H]5CCC(=O)NC5=O)cc3)CC4)cc2)C1] |
| 239/240-2b | 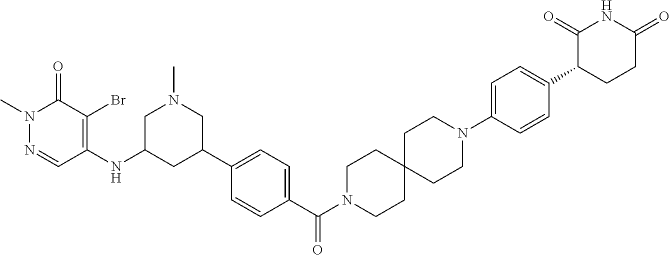 [SMILES: CN1CC(Nc2cnn(C)c(=O)c2Br)CC(c2ccc(C(=O)N3CCC4(CC3)CCN(c3ccc([C@H]5CCC(=O)NC5=O)cc3)CC4)cc2)C1] |
| 241 | 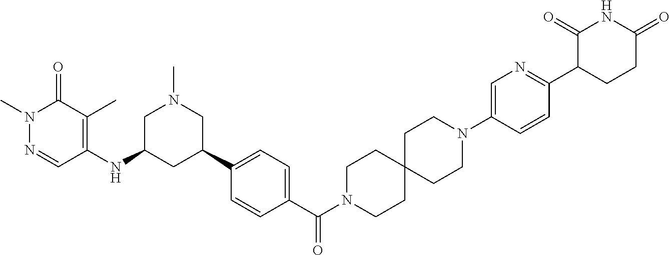 [SMILES: Cc1c(N[C@@H]2C[C@H](c3ccc(C(=O)N4CCC5(CC4)CCN(c4ccc(C6CCC(=O)NC6=O)nc4)CC5)cc3)CN(C)C2)cnn(C)c1=O] |
| 241a | 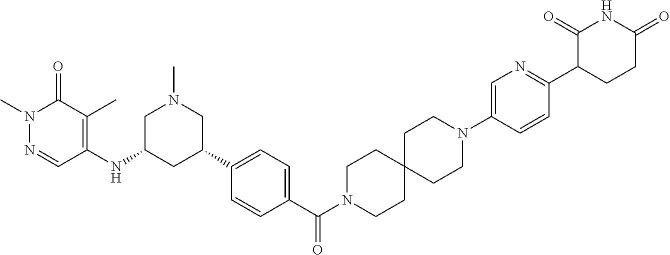 [SMILES: Cc1c(N[C@H]2C[C@@H](c3ccc(C(=O)N4CCC5(CC4)CCN(c4ccc(C6CCC(=O)NC6=O)nc4)CC5)cc3)CN(C)C2)cnn(C)c1=O] |
| 241b | 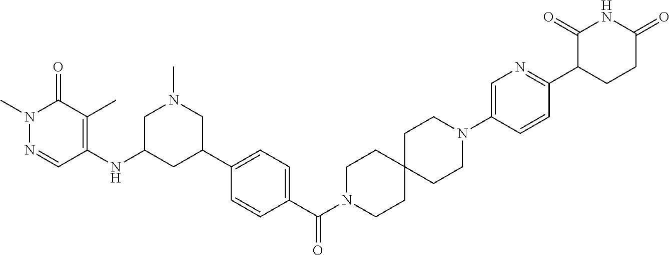 [SMILES: Cc1c(NC2CC(c3ccc(C(=O)N4CCC5(CC4)CCN(c4ccc(C6CCC(=O)NC6=O)nc4)CC5)cc3)CN(C)C2)cnn(C)c1=O] |
| 242 | 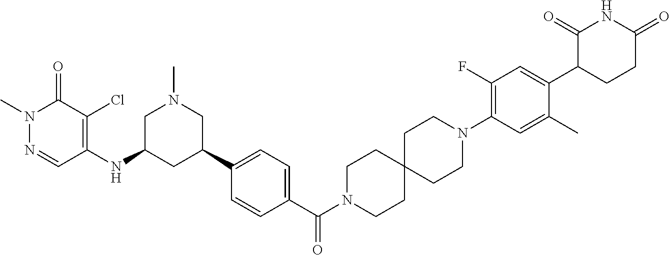 [SMILES: Cc1cc(N2CCC3(CCN(C(=O)c4ccc([C@H]5C[C@@H](Nc6cnn(C)c(=O)c6Cl)CN(C)C5)cc4)CC3)CC2)c(F)cc1C1CCC(=O)NC1=O] |
| 242a | 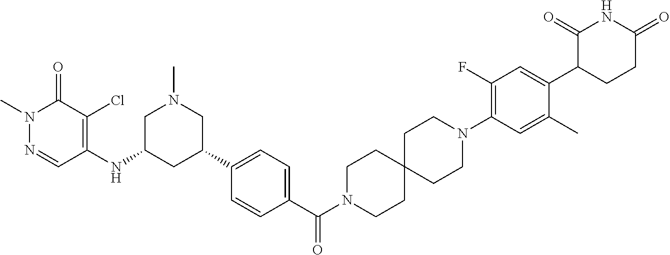 [SMILES: Cc1cc(N2CCC3(CCN(C(=O)c4ccc([C@@H]5C[C@H](Nc6cnn(C)c(=O)c6Cl)CN(C)C5)cc4)CC3)CC2)c(F)cc1C1CCC(=O)NC1=O] |
| 242b | 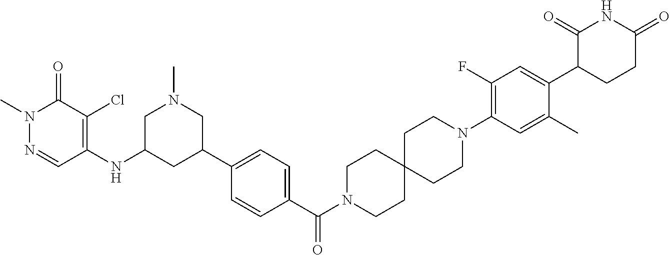 [SMILES: Cc1cc(N2CCC3(CCN(C(=O)c4ccc(C5CC(Nc6cnn(C)c(=O)c6Cl)CN(C)C5)cc4)CC3)CC2)c(F)cc1C1CCC(=O)NC1=O] |
| 243 | 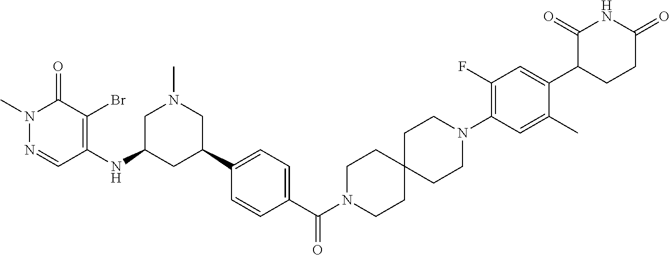 [SMILES: Cc1cc(N2CCC3(CCN(C(=O)c4ccc([C@H]5C[C@@H](Nc6cnn(C)c(=O)c6Br)CN(C)C5)cc4)CC3)CC2)c(F)cc1C1CCC(=O)NC1=O] |
| 243a | 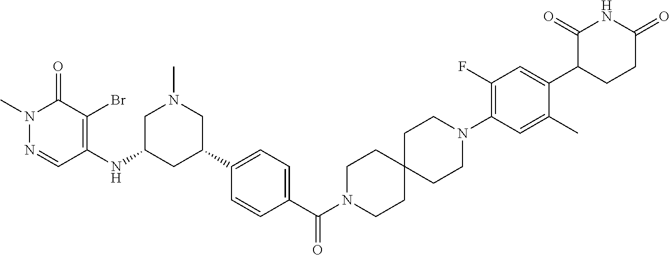 [SMILES: Cc1cc(N2CCC3(CCN(C(=O)c4ccc([C@@H]5C[C@H](Nc6cnn(C)c(=O)c6Br)CN(C)C5)cc4)CC3)CC2)c(F)cc1C1CCC(=O)NC1=O] |
| 243b | 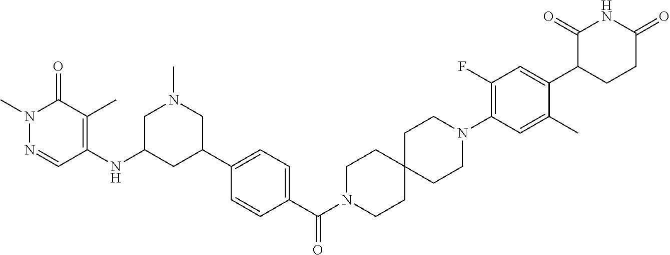 [SMILES: Cc1cc(N2CCC3(CCN(C(=O)c4ccc(C5CC(Nc6cnn(C)c(=O)c6C)CN(C)C5)cc4)CC3)CC2)c(F)cc1C1CCC(=O)NC1=O] |
| 244 | 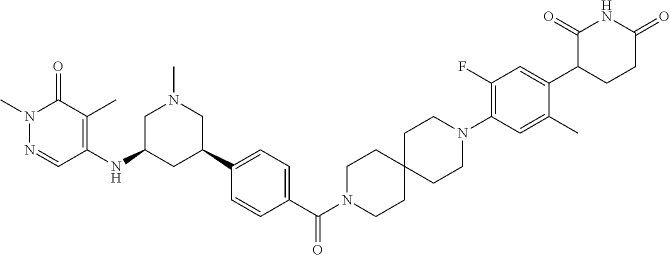 [SMILES: Cc1cc(N2CCC3(CCN(C(=O)c4ccc([C@H]5C[C@@H](Nc6cnn(C)c(=O)c6C)CN(C)C5)cc4)CC3)CC2)c(F)cc1C1CCC(=O)NC1=O] |
| 244a | 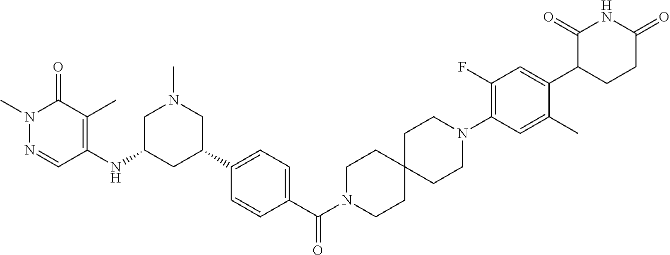 [SMILES: Cc1cc(N2CCC3(CCN(C(=O)c4ccc([C@@H]5C[C@H](Nc6cnn(C)c(=O)c6C)CN(C)C5)cc4)CC3)CC2)c(F)cc1C1CCC(=O)NC1=O] |
| 244b | 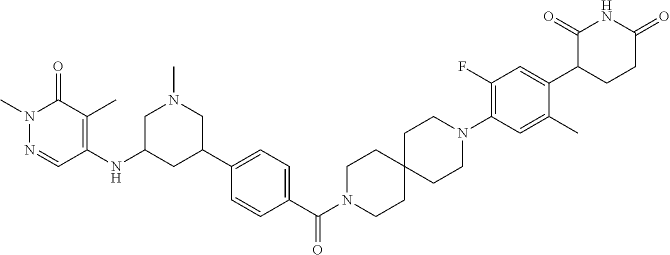 [SMILES: Cc1cc(N2CCC3(CCN(C(=O)c4ccc(C5CC(Nc6cnn(C)c(=O)c6C)CN(C)C5)cc4)CC3)CC2)c(F)cc1C1CCC(=O)NC1=O] |
| 245 | 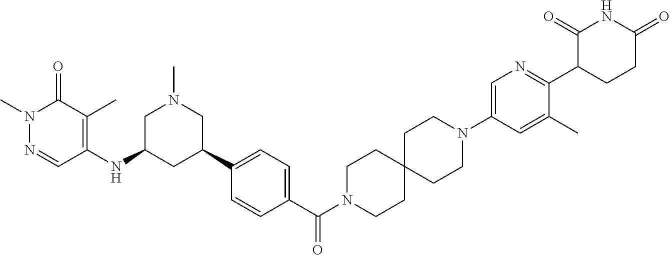 [SMILES: Cc1cc(N2CCC3(CCN(C(=O)c4ccc([C@H]5C[C@@H](Nc6cnn(C)c(=O)c6C)CN(C)C5)cc4)CC3)CC2)cnc1C1CCC(=O)NC1=O] |
| 245a | 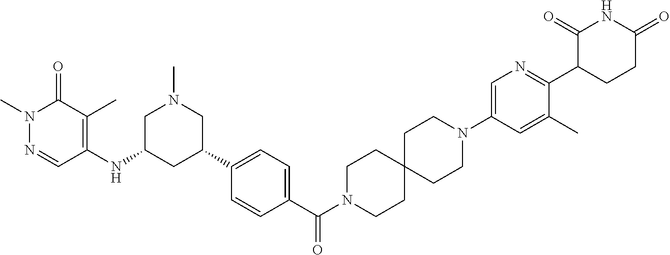 [SMILES: Cc1cc(N2CCC3(CCN(C(=O)c4ccc([C@@H]5C[C@H](Nc6cnn(C)c(=O)c6C)CN(C)C5)cc4)CC3)CC2)cnc1C1CCC(=O)NC1=O] |
| 245b | 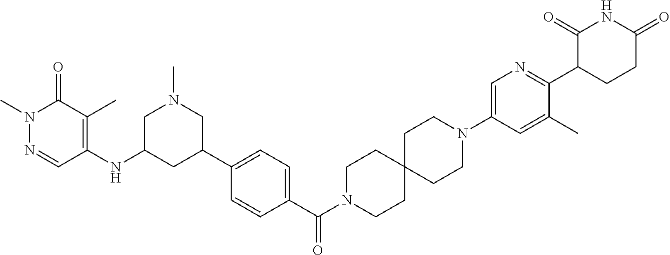 [SMILES: Cc1cc(N2CCC3(CCN(C(=O)c4ccc(C5CC(Nc6cnn(C)c(=O)c6C)CN(C)C5)cc4)CC3)CC2)cnc1C1CCC(=O)NC1=O] |
| 246 | 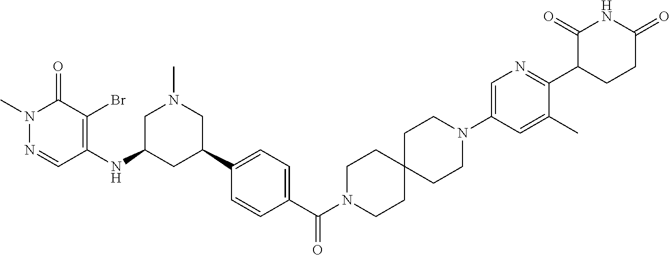 [SMILES: Cc1cc(N2CCC3(CCN(C(=O)c4ccc([C@H]5C[C@@H](Nc6cnn(C)c(=O)c6Br)CN(C)C5)cc4)CC3)CC2)cnc1C1CCC(=O)NC1=O] |
| 246a | 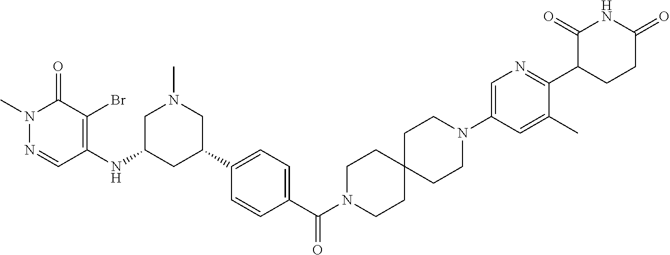 [SMILES: Cc1cc(N2CCC3(CCN(C(=O)c4ccc([C@@H]5C[C@H](Nc6cnn(C)c(=O)c6Br)CN(C)C5)cc4)CC3)CC2)cnc1C1CCC(=O)NC1=O] |
| 246b | 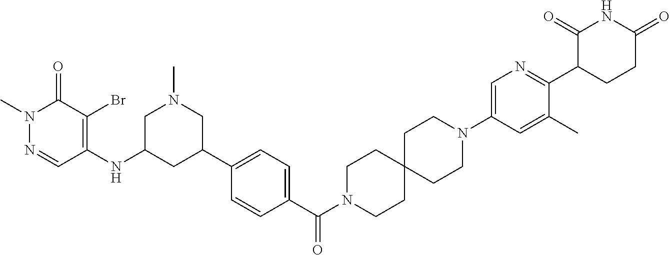 [SMILES: Cc1cc(N2CCC3(CCN(C(=O)c4ccc(C5CC(Nc6cnn(C)c(=O)c6Br)CN(C)C5)cc4)CC3)CC2)cnc1C1CCC(=O)NC1=O] |
| 247 | 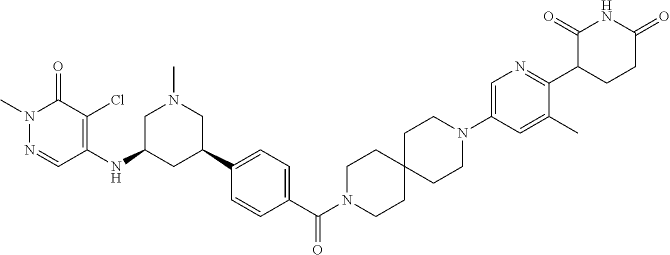 [SMILES: Cc1cc(N2CCC3(CCN(C(=O)c4ccc([C@H]5C[C@@H](Nc6cnn(C)c(=O)c6Cl)CN(C)C5)cc4)CC3)CC2)cnc1C1CCC(=O)NC1=O] |
| 247a | 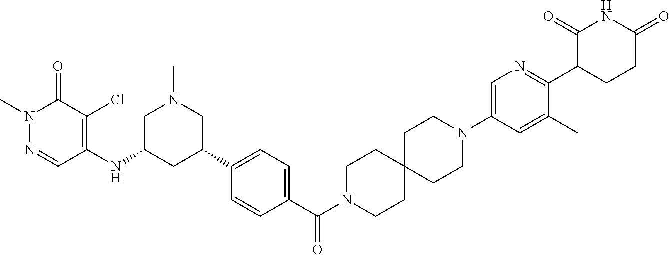 [SMILES: Cc1cc(N2CCC3(CCN(C(=O)c4ccc([C@@H]5C[C@H](Nc6cnn(C)c(=O)c6Cl)CN(C)C5)cc4)CC3)CC2)cnc1C1CCC(=O)NC1=O] |
| 247b | 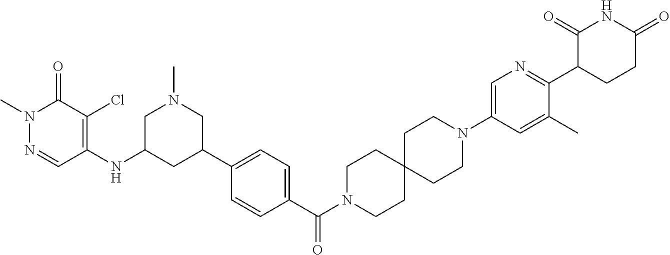 [SMILES: Cc1cc(N2CCC3(CCN(C(=O)c4ccc(C5CC(Nc6cnn(C)c(=O)c6Cl)CN(C)C5)cc4)CC3)CC2)cnc1C1CCC(=O)NC1=O] |
| 248 | 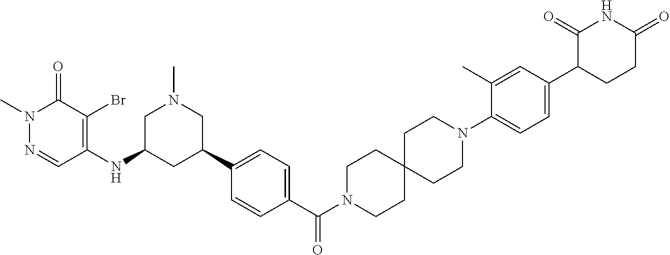 [SMILES: Cc1cc(C2CCC(=O)NC2=O)ccc1N1CCC2(CCN(C(=O)c3ccc([C@H]4C[C@@H](Nc5cnn(C)c(=O)c5Br)CN(C)C4)cc3)CC2)CC1] |
| 248a | 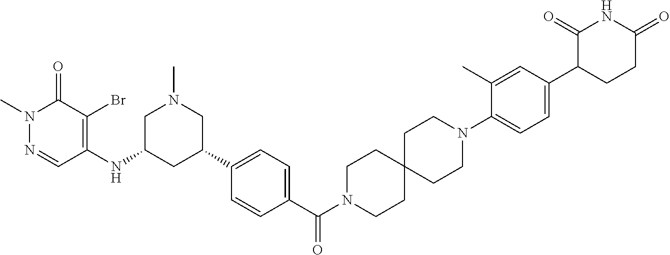 [SMILES: Cc1cc(C2CCC(=O)NC2=O)ccc1N1CCC2(CCN(C(=O)c3ccc([C@@H]4C[C@H](Nc5cnn(C)c(=O)c5Br)CN(C)C4)cc3)CC2)CC1] |
| 248b | 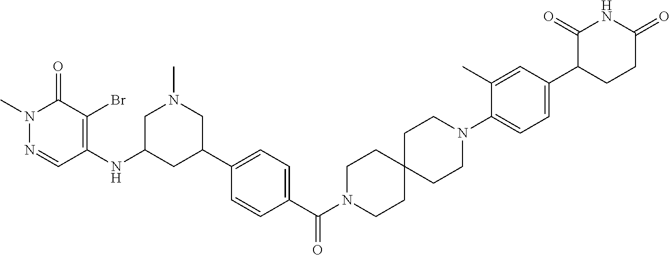 [SMILES: Cc1cc(C2CCC(=O)NC2=O)ccc1N1CCC2(CCN(C(=O)c3ccc(C4CC(Nc5cnn(C)c(=O)c5Br)CN(C)C4)cc3)CC2)CC1] |
| 249 | 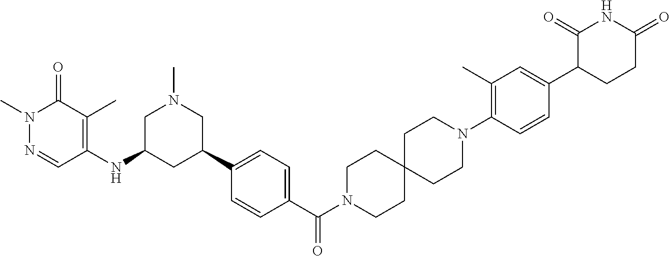 [SMILES: Cc1cc(C2CCC(=O)NC2=O)ccc1N1CCC2(CCN(C(=O)c3ccc([C@H]4C[C@@H](Nc5cnn(C)c(=O)c5C)CN(C)C4)cc3)CC2)CC1] |
| 249a | 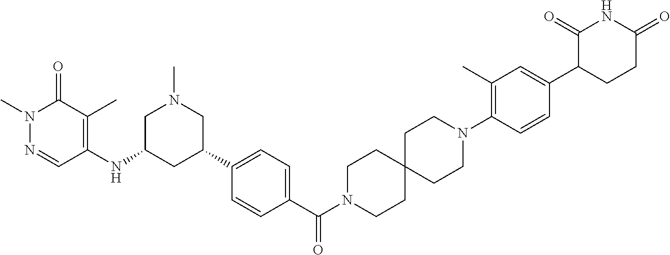 [SMILES: Cc1cc(C2CCC(=O)NC2=O)ccc1N1CCC2(CCN(C(=O)c3ccc([C@@H]4C[C@H](Nc5cnn(C)c(=O)c5C)CN(C)C4)cc3)CC2)CC1] |
| 249b | 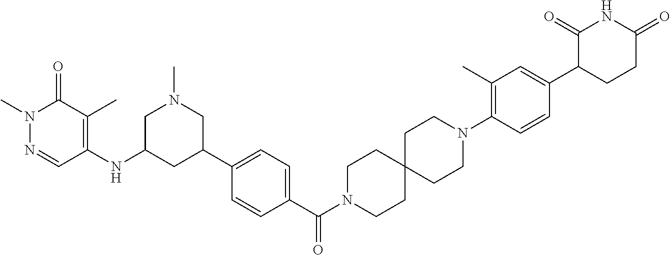 [SMILES: Cc1cc(C2CCC(=O)NC2=O)ccc1N1CCC2(CCN(C(=O)c3ccc(C4CC(Nc5cnn(C)c(=O)c5C)CN(C)C4)cc3)CC2)CC1] |
| 250 | 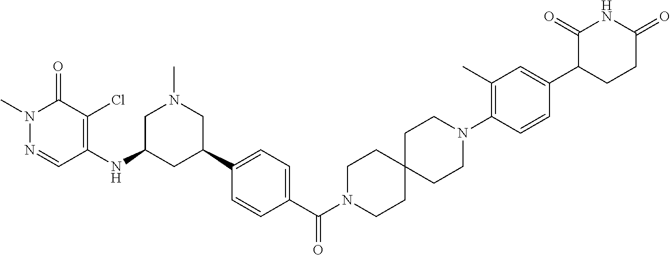 [SMILES: Cc1cc(C2CCC(=O)NC2=O)ccc1N1CCC2(CCN(C(=O)c3ccc([C@H]4C[C@@H](Nc5cnn(C)c(=O)c5Cl)CN(C)C4)cc3)CC2)CC1] |
| 250a | 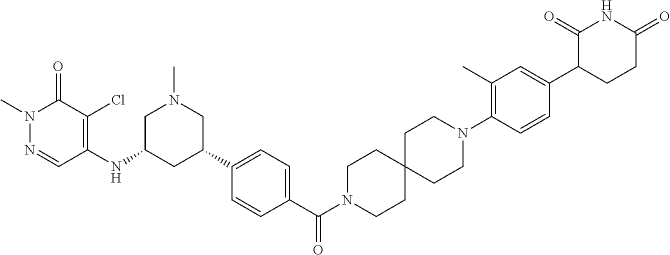 [SMILES: Cc1cc(C2CCC(=O)NC2=O)ccc1N1CCC2(CCN(C(=O)c3ccc([C@@H]4C[C@H](Nc5cnn(C)c(=O)c5Cl)CN(C)C4)cc3)CC2)CC1] |
| 250b | 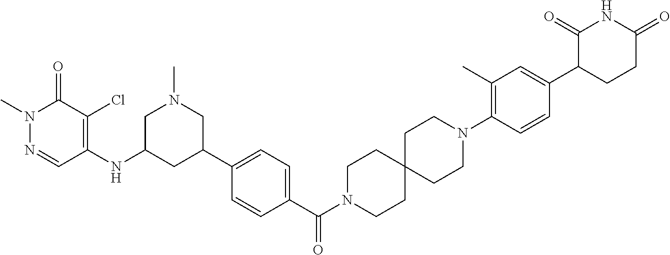 [SMILES: Cc1cc(C2CCC(=O)NC2=O)ccc1N1CCC2(CCN(C(=O)c3ccc(C4CC(Nc5cnn(C)c(=O)c5Cl)CN(C)C4)cc3)CC2)CC1] |
| 251 | 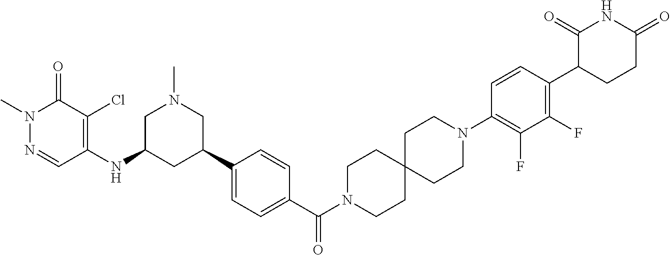 [SMILES: CN1C[C@H](Nc2cnn(C)c(=O)c2Cl)C[C@H](c2ccc(C(=O)N3CCC4(CC3)CCN(c3ccc(C5CCC(=O)NC5=O)c(F)c3F)CC4)cc2)C1] |
| 251a | 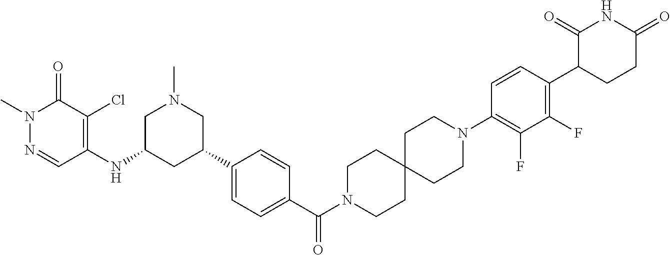 [SMILES: CN1C[C@@H](Nc2cnn(C)c(=O)c2Cl)C[C@@H](c2ccc(C(=O)N3CCC4(CC3)CCN(c3ccc(C5CCC(=O)NC5=O)c(F)c3F)CC4)cc2)C1] |
| 251b | 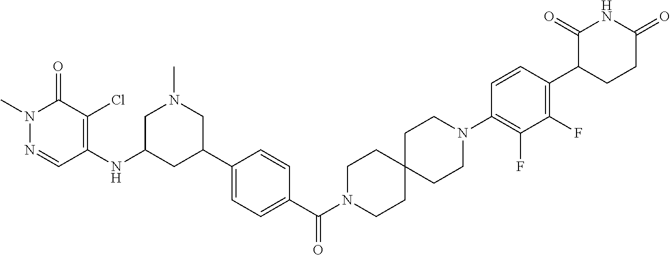 [SMILES: CN1CC(Nc2cnn(C)c(=O)c2Cl)CC(c2ccc(C(=O)N3CCC4(CC3)CCN(c3ccc(C5CCC(=O)NC5=O)c(F)c3F)CC4)cc2)C1] |
| 252 | 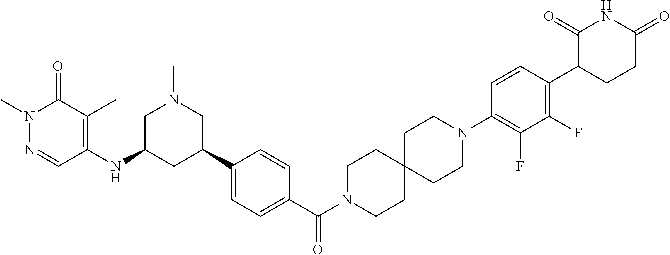 [SMILES: Cc1c(N[C@@H]2C[C@H](c3ccc(C(=O)N4CCC5(CC4)CCN(c4ccc(C6CCC(=O)NC6=O)c(F)c4F)CC5)cc3)CN(C)C2)cnn(C)c1=O] |
| 252a | 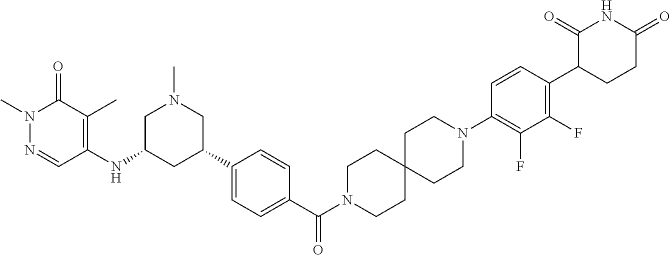 [SMILES: Cc1c(N[C@H]2C[C@@H](c3ccc(C(=O)N4CCC5(CC4)CCN(c4ccc(C6CCC(=O)NC6=O)c(F)c4F)CC5)cc3)CN(C)C2)cnn(C)c1=O] |
| 252b | 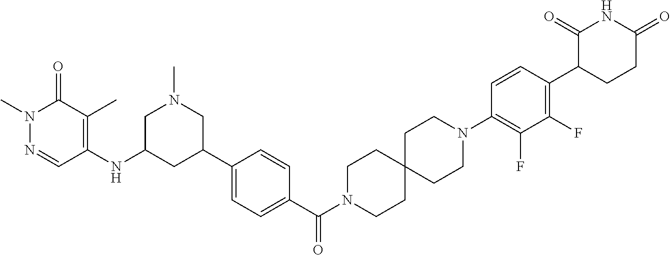 [SMILES: Cc1c(NC2CC(c3ccc(C(=O)N4CCC5(CC4)CCN(c4ccc(C6CCC(=O)NC6=O)c(F)c4F)CC5)cc3)CN(C)C2)cnn(C)c1=O] |
| 253 | 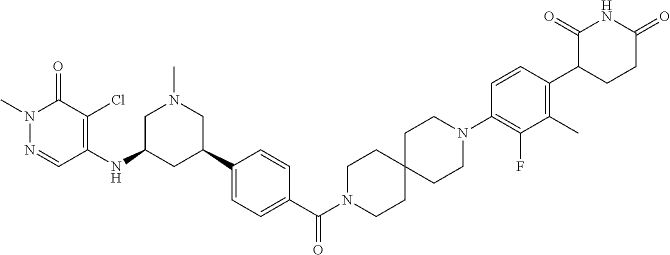 [SMILES: Cc1c(C2CCC(=O)NC2=O)ccc(N2CCC3(CCN(C(=O)c4ccc([C@H]5C[C@@H](Nc6cnn(C)c(=O)c6Cl)CN(C)C5)cc4)CC3)CC2)c1F] |
| 253a | 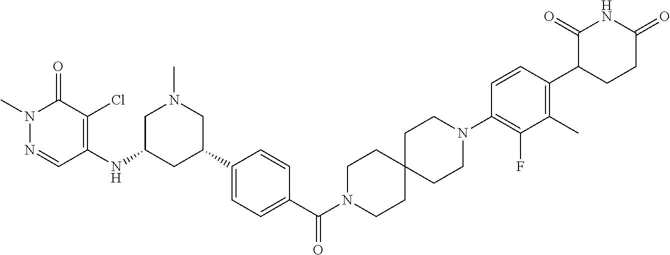 [SMILES: Cc1c(C2CCC(=O)NC2=O)ccc(N2CCC3(CCN(C(=O)c4ccc([C@@H]5C[C@H](Nc6cnn(C)c(=O)c6Cl)CN(C)C5)cc4)CC3)CC2)c1F] |
| 253b | 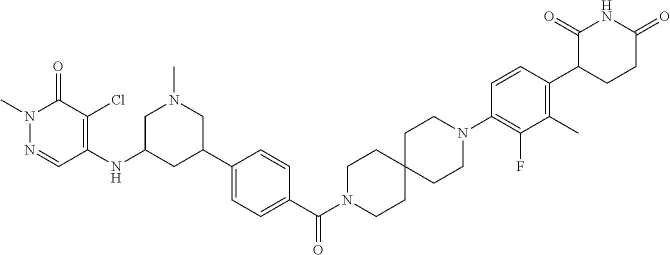 [SMILES: Cc1c(C2CCC(=O)NC2=O)ccc(N2CCC3(CCN(C(=O)c4ccc(C5CC(Nc6cnn(C)c(=O)c6Cl)CN(C)C5)cc4)CC3)CC2)c1F] |
| 254 | 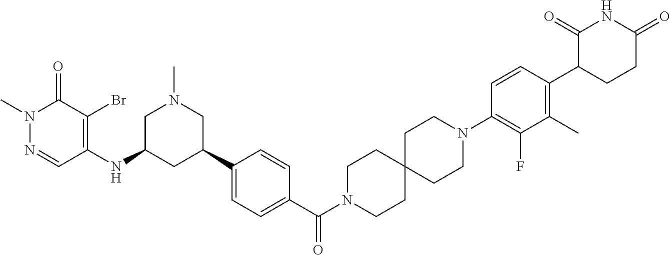 [SMILES: Cc1c(C2CCC(=O)NC2=O)ccc(N2CCC3(CCN(C(=O)c4ccc([C@H]5C[C@@H](Nc6cnn(C)c(=O)c6Br)CN(C)C5)cc4)CC3)CC2)c1F] |
| 254a | 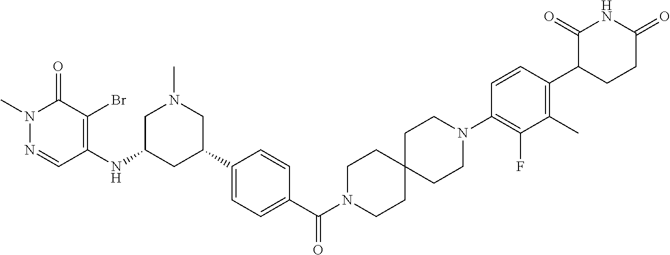 [SMILES: Cc1c(C2CCC(=O)NC2=O)ccc(N2CCC3(CCN(C(=O)c4ccc([C@@H]5C[C@H](Nc6cnn(C)c(=O)c6Br)CN(C)C5)cc4)CC3)CC2)c1F] |
| 254b | 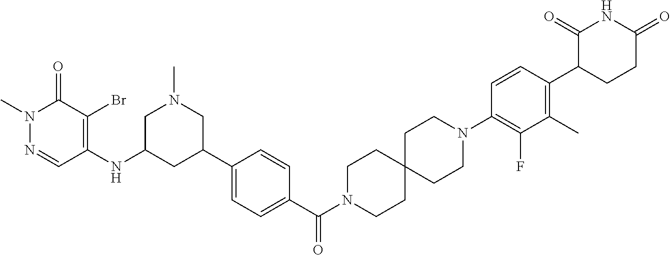 [SMILES: Cc1c(C2CCC(=O)NC2=O)ccc(N2CCC3(CCN(C(=O)c4ccc(C5CC(Nc6cnn(C)c(=O)c6Br)CN(C)C5)cc4)CC3)CC2)c1F] |
| 255 | 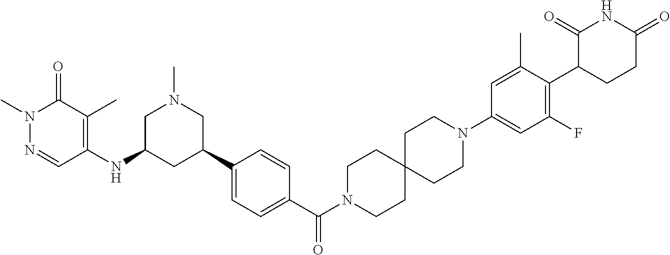 [SMILES: Cc1cc(N2CCC3(CCN(C(=O)c4ccc([C@H]5C[C@@H](Nc6cnn(C)c(=O)c6C)CN(C)C5)cc4)CC3)CC2)cc(F)c1C1CCC(=O)NC1=O] |
| 255a | 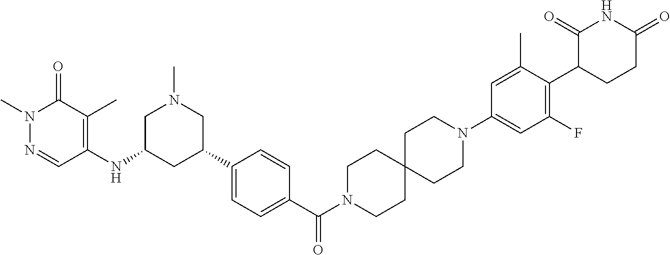 [SMILES: Cc1cc(N2CCC3(CCN(C(=O)c4ccc([C@@H]5C[C@H](Nc6cnn(C)c(=O)c6C)CN(C)C5)cc4)CC3)CC2)cc(F)c1C1CCC(=O)NC1=O] |
| 255b | 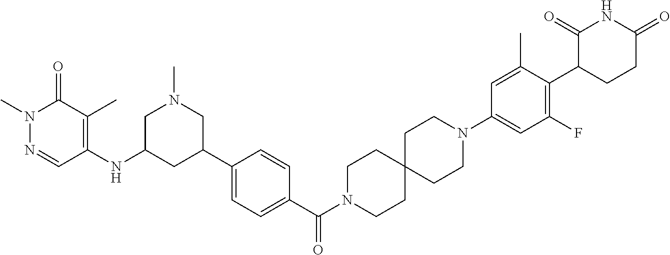 [SMILES: Cc1cc(N2CCC3(CCN(C(=O)c4ccc(C5CC(Nc6cnn(C)c(=O)c6C)CN(C)C5)cc4)CC3)CC2)cc(F)c1C1CCC(=O)NC1=O] |
| 256 | 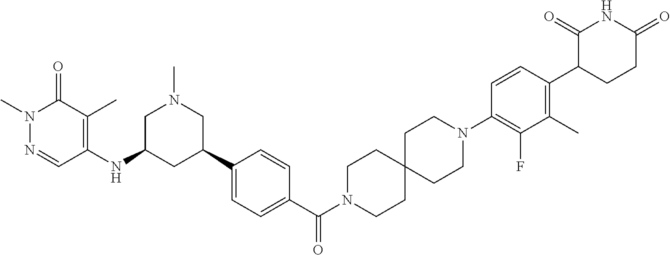 [SMILES: Cc1c(C2CCC(=O)NC2=O)ccc(N2CCC3(CCN(C(=O)c4ccc([C@H]5C[C@@H](Nc6cnn(C)c(=O)c6C)CN(C)C5)cc4)CC3)CC2)c1F] |
| 256a | 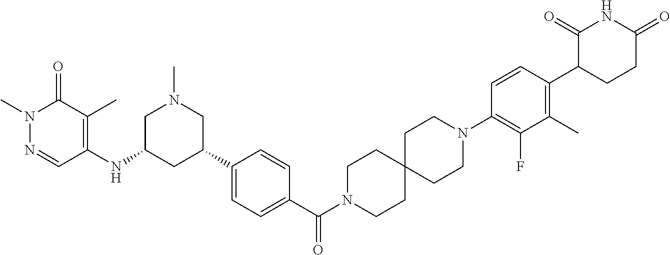 [SMILES: Cc1c(C2CCC(=O)NC2=O)ccc(N2CCC3(CCN(C(=O)c4ccc([C@@H]5C[C@H](Nc6cnn(C)c(=O)c6C)CN(C)C5)cc4)CC3)CC2)c1F] |
| 256b | 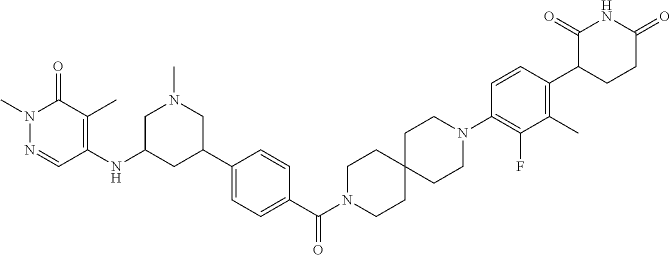 [SMILES: Cc1c(C2CCC(=O)NC2=O)ccc(N2CCC3(CCN(C(=O)c4ccc(C5CC(Nc6cnn(C)c(=O)c6C)CN(C)C5)cc4)CC3)CC2)c1F] |
| 257 | 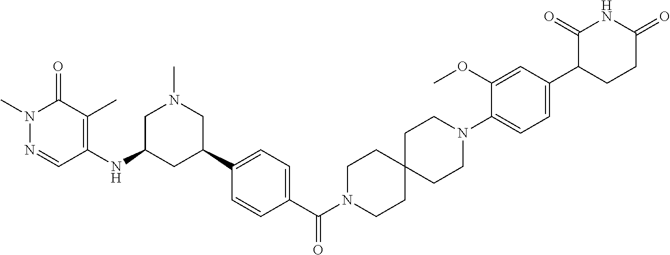 [SMILES: COc1cc(C2CCC(=O)NC2=O)ccc1N1CCC2(CCN(C(=O)c3ccc([C@H]4C[C@@H](Nc5cnn(C)c(=O)c5C)CN(C)C4)cc3)CC2)CC1] |
| 257a | 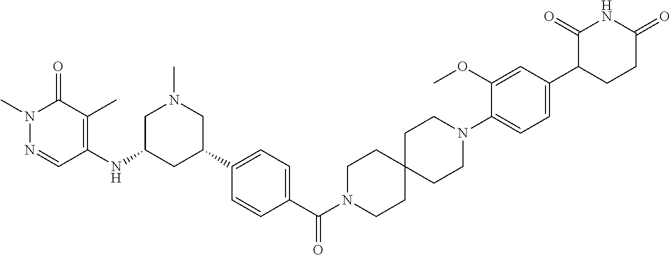 [SMILES: COc1cc(C2CCC(=O)NC2=O)ccc1N1CCC2(CCN(C(=O)c3ccc([C@@H]4C[C@H](Nc5cnn(C)c(=O)c5C)CN(C)C4)cc3)CC2)CC1] |
| 257b | 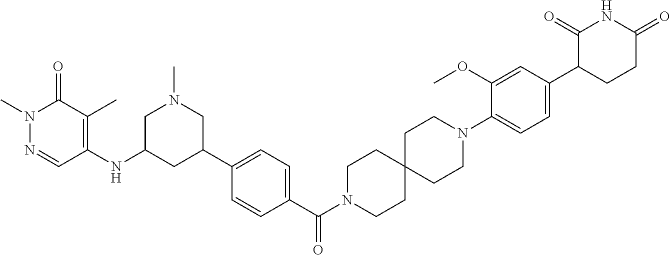 [SMILES: COc1cc(C2CCC(=O)NC2=O)ccc1N1CCC2(CCN(C(=O)c3ccc(C4CC(Nc5cnn(C)c(=O)c5C)CN(C)C4)cc3)CC2)CC1] |
| 258 | 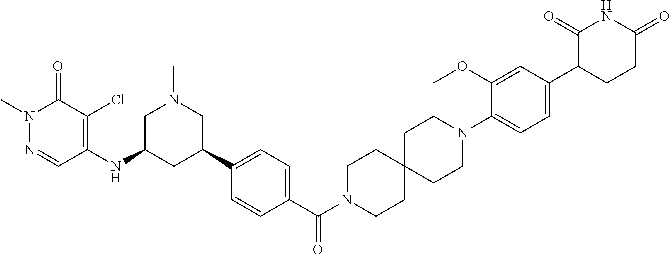 [SMILES: COc1cc(C2CCC(=O)NC2=O)ccc1N1CCC2(CCN(C(=O)c3ccc([C@H]4C[C@@H](Nc5cnn(C)c(=O)c5Cl)CN(C)C4)cc3)CC2)CC1] |
| 258a | 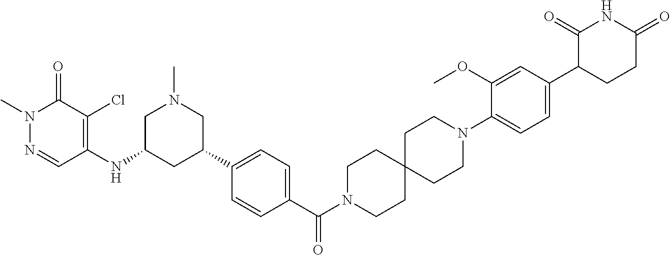 [SMILES: COc1cc(C2CCC(=O)NC2=O)ccc1N1CCC2(CCN(C(=O)c3ccc([C@@H]4C[C@H](Nc5cnn(C)c(=O)c5Cl)CN(C)C4)cc3)CC2)CC1] |
| 258b | 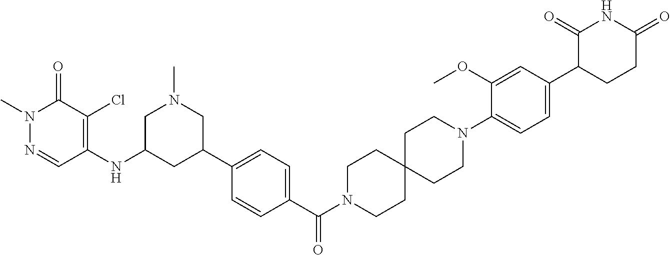 [SMILES: COc1cc(C2CCC(=O)NC2=O)ccc1N1CCC2(CCN(C(=O)c3ccc(C4CC(Nc5cnn(C)c(=O)c5Cl)CN(C)C4)cc3)CC2)CC1] |
| 259 | 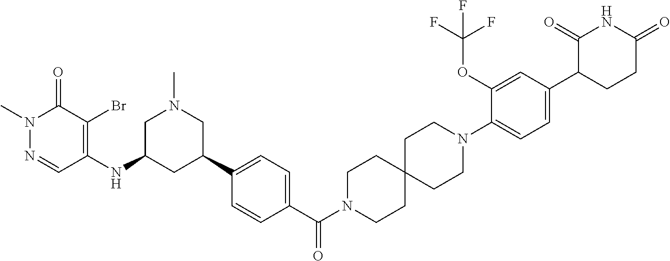 [SMILES: CN1C[C@H](Nc2cnn(C)c(=O)c2Br)C[C@H](c2ccc(C(=O)N3CCC4(CC3)CCN(c3ccc(C5CCC(=O)NC5=O)cc3OC(F)(F)F)CC4)cc2)C1] |
| 259a | 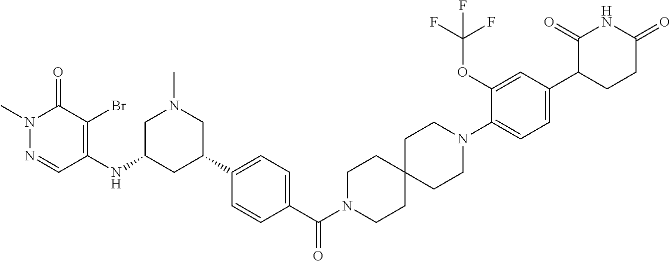 [SMILES: CN1C[C@@H](Nc2cnn(C)c(=O)c2Br)C[C@@H](c2ccc(C(=O)N3CCC4(CC3)CCN(c3ccc(C5CCC(=O)NC5=O)cc3OC(F)(F)F)CC4)cc2)C1] |
| 259b | 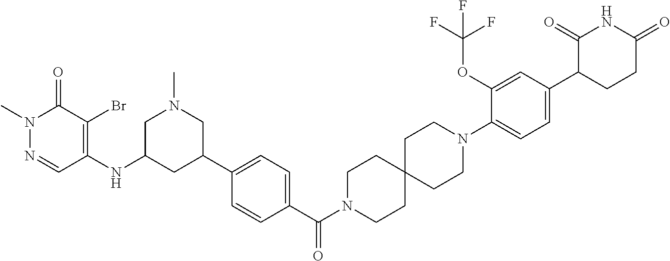 [SMILES: CN1CC(Nc2cnn(C)c(=O)c2Br)CC(c2ccc(C(=O)N3CCC4(CC3)CCN(c3ccc(C5CCC(=O)NC5=O)cc3OC(F)(F)F)CC4)cc2)C1] |
| 260 | 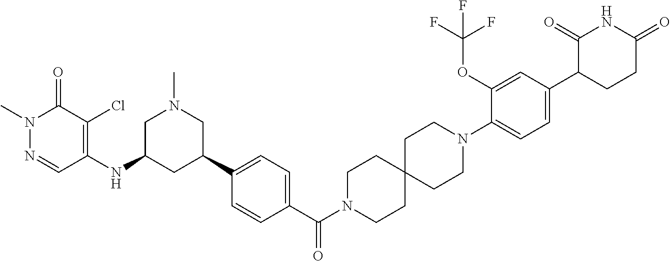 [SMILES: CN1C[C@H](Nc2cnn(C)c(=O)c2Cl)C[C@H](c2ccc(C(=O)N3CCC4(CC3)CCN(c3ccc(C5CCC(=O)NC5=O)cc3OC(F)(F)F)CC4)cc2)C1] |
| 260a | 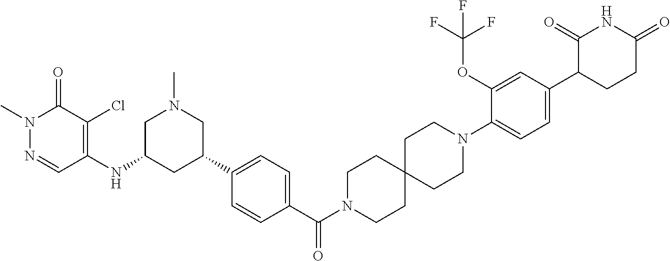 [SMILES: CN1C[C@@H](Nc2cnn(C)c(=O)c2Cl)C[C@@H](c2ccc(C(=O)N3CCC4(CC3)CCN(c3ccc(C5CCC(=O)NC5=O)cc3OC(F)(F)F)CC4)cc2)C1] |
| 260b | 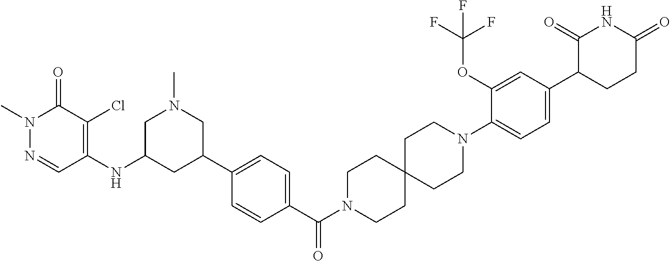 [SMILES: CN1CC(Nc2cnn(C)c(=O)c2Cl)CC(c2ccc(C(=O)N3CCC4(CC3)CCN(c3ccc(C5CCC(=O)NC5=O)cc3OC(F)(F)F)CC4)cc2)C1] |
| 261 | 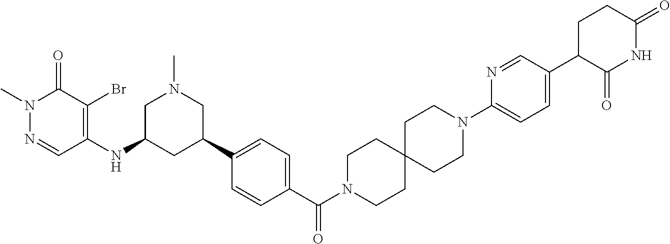 [SMILES: CN1C[C@H](Nc2cnn(C)c(=O)c2Br)C[C@H](c2ccc(C(=O)N3CCC4(CC3)CCN(c3ccc(C5CCC(=O)NC5=O)cn3)CC4)cc2)C1] |
| 261a | 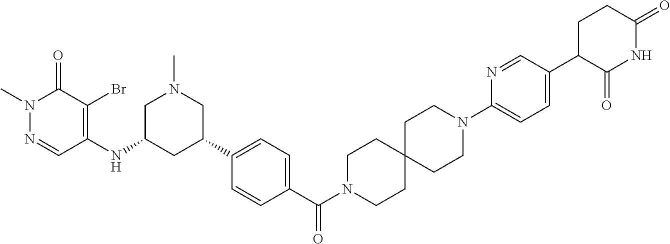 [SMILES: CN1C[C@@H](Nc2cnn(C)c(=O)c2Br)C[C@@H](c2ccc(C(=O)N3CCC4(CC3)CCN(c3ccc(C5CCC(=O)NC5=O)cn3)CC4)cc2)C1] |
| 261b | 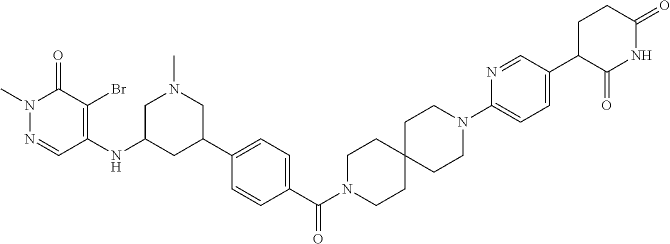 [SMILES: CN1CC(Nc2cnn(C)c(=O)c2Br)CC(c2ccc(C(=O)N3CCC4(CC3)CCN(c3ccc(C5CCC(=O)NC5=O)cn3)CC4)cc2)C1] |
| 262 | 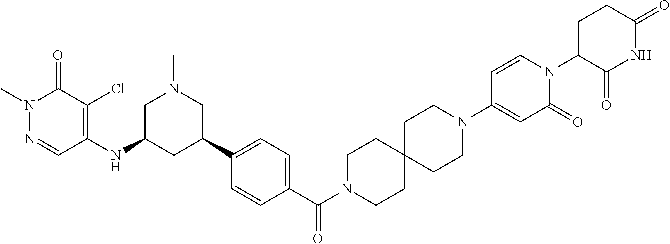 [SMILES: CN1C[C@H](Nc2cnn(C)c(=O)c2Cl)C[C@H](c2ccc(C(=O)N3CCC4(CC3)CCN(c3ccn(C5CCC(=O)NC5=O)c(=O)c3)CC4)cc2)C1] |
| 262a | 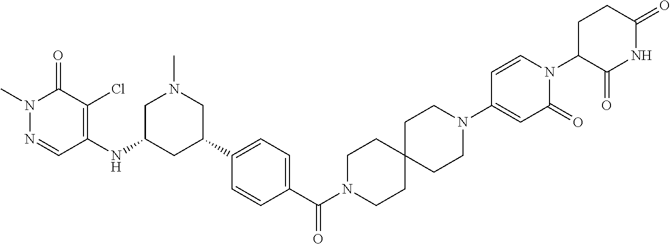 [SMILES: CN1C[C@@H](Nc2cnn(C)c(=O)c2Cl)C[C@@H](c2ccc(C(=O)N3CCC4(CC3)CCN(c3ccn(C5CCC(=O)NC5=O)c(=O)c3)CC4)cc2)C1] |
| 262b | 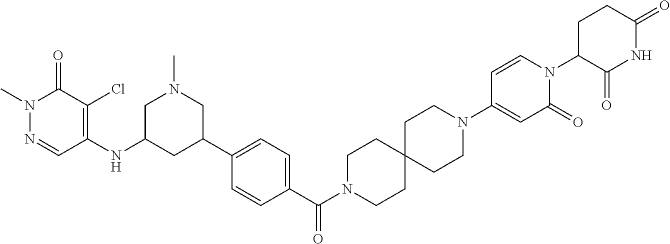 [SMILES: CN1CC(Nc2cnn(C)c(=O)c2Cl)CC(c2ccc(C(=O)N3CCC4(CC3)CCN(c3ccn(C5CCC(=O)NC5=O)c(=O)c3)CC4)cc2)C1] |
| 263 | 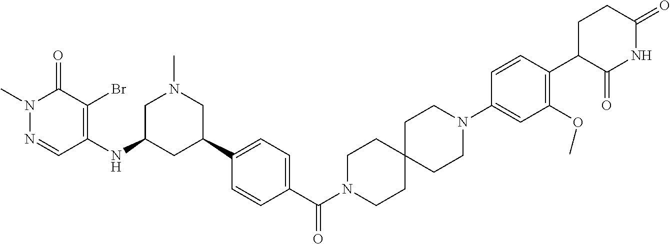 [SMILES: COc1cc(N2CCC3(CCN(C(=O)c4ccc([C@H]5C[C@@H](Nc6cnn(C)c(=O)c6Br)CN(C)C5)cc4)CC3)CC2)ccc1C1CCC(=O)NC1=O] |
| 263a | 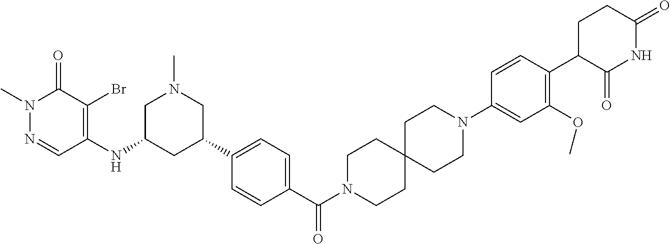 [SMILES: COc1cc(N2CCC3(CCN(C(=O)c4ccc([C@@H]5C[C@H](Nc6cnn(C)c(=O)c6Br)CN(C)C5)cc4)CC3)CC2)ccc1C1CCC(=O)NC1=O] |
| 263b | 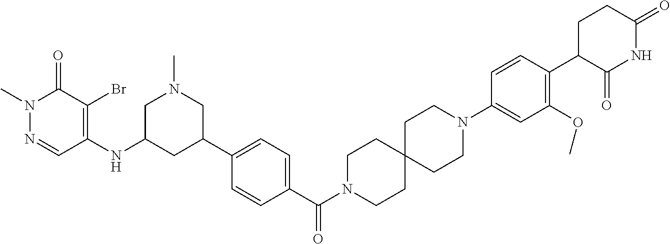 [SMILES: COc1cc(N2CCC3(CCN(C(=O)c4ccc(C5CC(Nc6cnn(C)c(=O)c6Br)CN(C)C5)cc4)CC3)CC2)ccc1C1CCC(=O)NC1=O] |
| 264 | 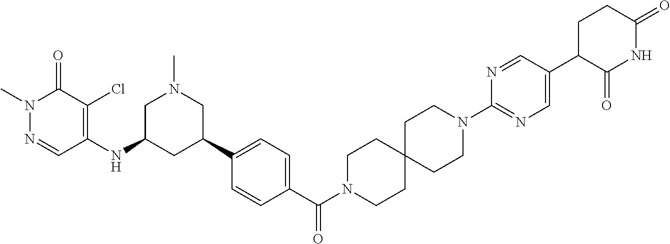 [SMILES: CN1C[C@H](Nc2cnn(C)c(=O)c2Cl)C[C@H](c2ccc(C(=O)N3CCC4(CC3)CCN(c3ncc(C5CCC(=O)NC5=O)cn3)CC4)cc2)C1] |
| 264a | 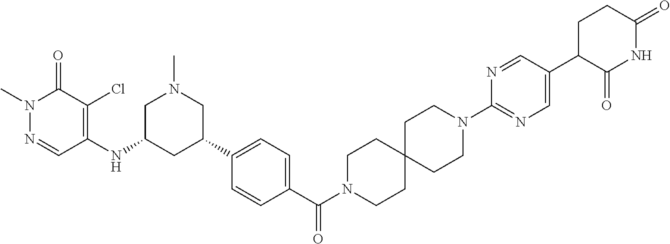 [SMILES: CN1C[C@@H](Nc2cnn(C)c(=O)c2Cl)C[C@@H](c2ccc(C(=O)N3CCC4(CC3)CCN(c3ncc(C5CCC(=O)NC5=O)cn3)CC4)cc2)C1] |
| 264b | 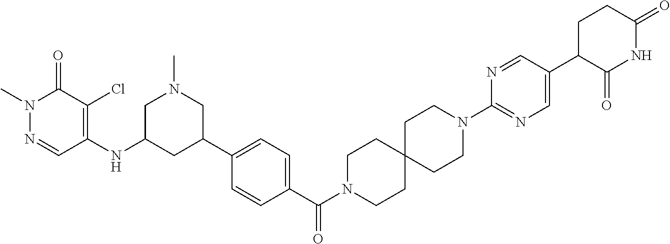 [SMILES: CN1CC(Nc2cnn(C)c(=O)c2Cl)CC(c2ccc(C(=O)N3CCC4(CC3)CCN(c3ncc(C5CCC(=O)NC5=O)cn3)CC4)cc2)C1] |
| 265 | 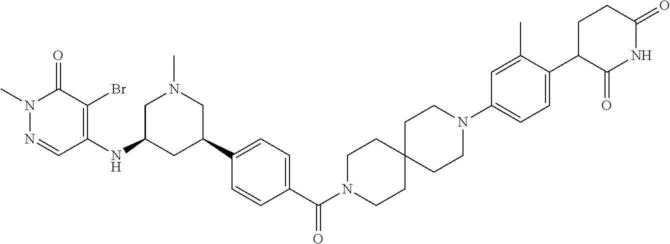 [SMILES: Cc1cc(N2CCC3(CCN(C(=O)c4ccc([C@H]5C[C@@H](Nc6cnn(C)c(=O)c6Br)CN(C)C5)cc4)CC3)CC2)ccc1C1CCC(=O)NC1=O] |
| 265a | 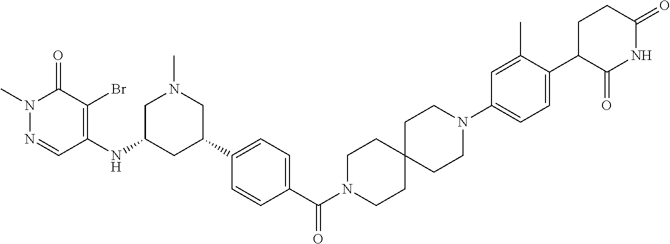 [SMILES: Cc1cc(N2CCC3(CCN(C(=O)c4ccc([C@@H]5C[C@H](Nc6cnn(C)c(=O)c6Br)CN(C)C5)cc4)CC3)CC2)ccc1C1CCC(=O)NC1=O] |
| 265b | 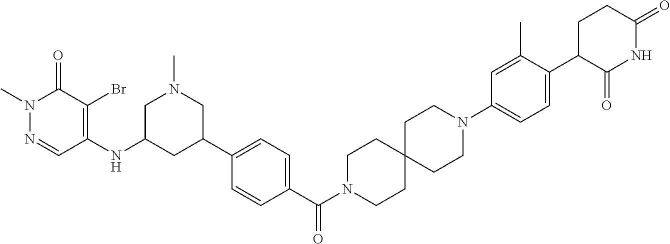 [SMILES: Cc1cc(N2CCC3(CCN(C(=O)c4ccc(C5CC(Nc6cnn(C)c(=O)c6Br)CN(C)C5)cc4)CC3)CC2)ccc1C1CCC(=O)NC1=O] |
| 266 | 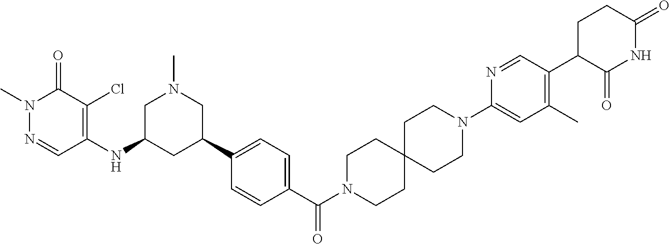 [SMILES: Cc1cc(N2CCC3(CCN(C(=O)c4ccc([C@H]5C[C@@H](Nc6cnn(C)c(=O)c6Cl)CN(C)C5)cc4)CC3)CC2)ncc1C1CCC(=O)NC1=O] |
| 266a | 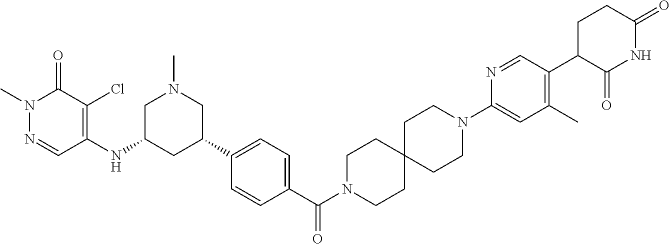 [SMILES: Cc1cc(N2CCC3(CCN(C(=O)c4ccc([C@@H]5C[C@H](Nc6cnn(C)c(=O)c6Cl)CN(C)C5)cc4)CC3)CC2)ncc1C1CCC(=O)NC1=O] |
| 266b | 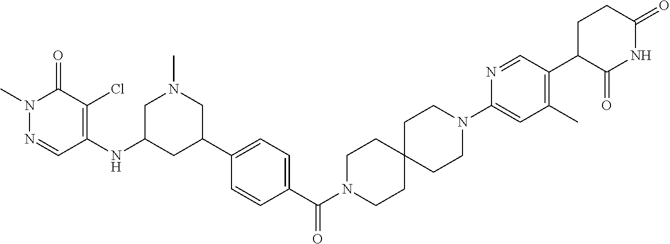 [SMILES: Cc1cc(N2CCC3(CCN(C(=O)c4ccc(C5CC(Nc6cnn(C)c(=O)c6Cl)CN(C)C5)cc4)CC3)CC2)ncc1C1CCC(=O)NC1=O] |
| 267 | 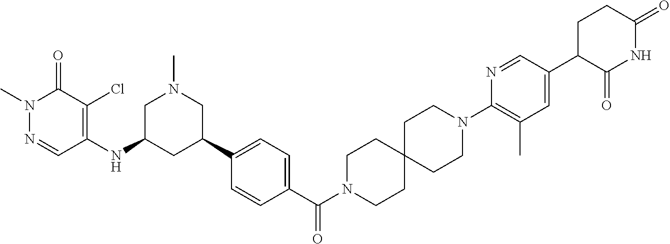 [SMILES: Cc1cc(C2CCC(=O)NC2=O)cnc1N1CCC2(CCN(C(=O)c3ccc([C@H]4C[C@@H](Nc5cnn(C)c(=O)c5Cl)CN(C)C4)cc3)CC2)CC1] |
| 267a | 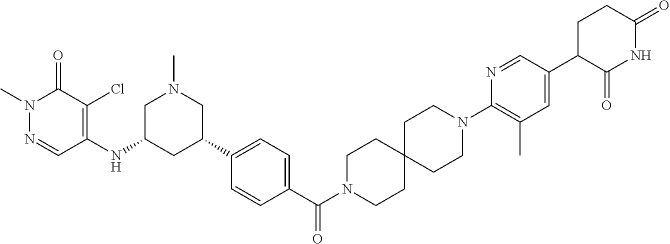 [SMILES: Cc1cc(C2CCC(=O)NC2=O)cnc1N1CCC2(CCN(C(=O)c3ccc([C@@H]4C[C@H](Nc5cnn(C)c(=O)c5Cl)CN(C)C4)cc3)CC2)CC1] |
| 267b | 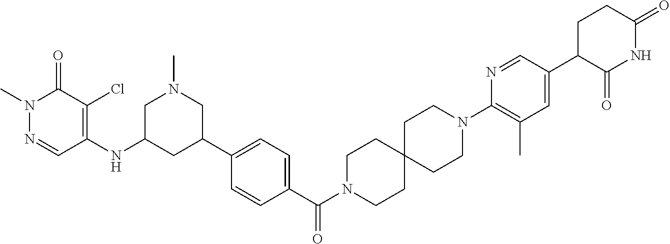 [SMILES: Cc1cc(C2CCC(=O)NC2=O)cnc1N1CCC2(CCN(C(=O)c3ccc(C4CC(Nc5cnn(C)c(=O)c5Cl)CN(C)C4)cc3)CC2)CC1] |
| 268 | 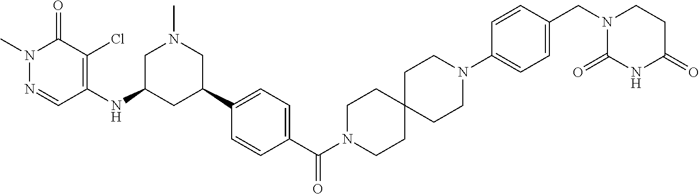 [SMILES: CN1C[C@H](Nc2cnn(C)c(=O)c2Cl)C[C@H](c2ccc(C(=O)N3CCC4(CC3)CCN(c3ccc(CN5CCC(=O)NC5=O)cc3)CC4)cc2)C1] |
| 268a | 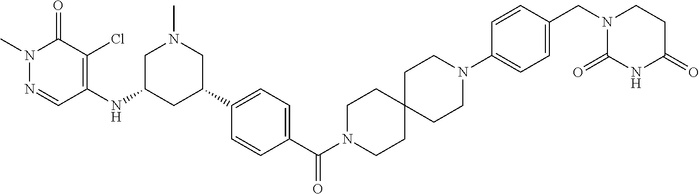 [SMILES: CN1C[C@@H](Nc2cnn(C)c(=O)c2Cl)C[C@@H](c2ccc(C(=O)N3CCC4(CC3)CCN(c3ccc(CN5CCC(=O)NC5=O)cc3)CC4)cc2)C1] |
| 268b | 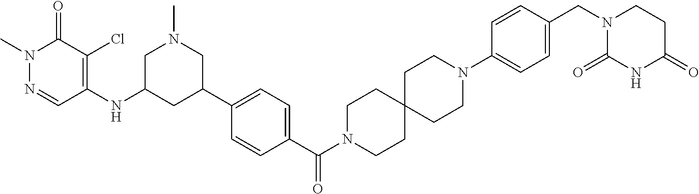 [SMILES: CN1CC(Nc2cnn(C)c(=O)c2Cl)CC(c2ccc(C(=O)N3CCC4(CC3)CCN(c3ccc(CN5CCC(=O)NC5=O)cc3)CC4)cc2)C1] |
| 269 | 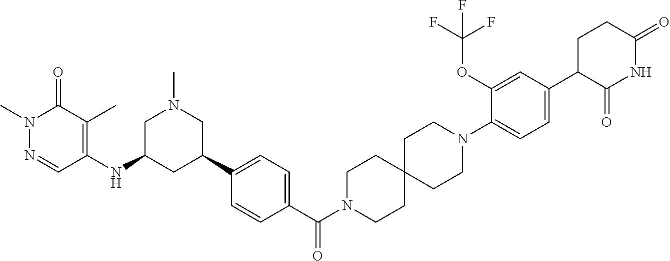 [SMILES: Cc1c(N[C@@H]2C[C@H](c3ccc(C(=O)N4CCC5(CC4)CCN(c4ccc(C6CCC(=O)NC6=O)cc4OC(F)(F)F)CC5)cc3)CN(C)C2)cnn(C)c1=O] |
| 269a | 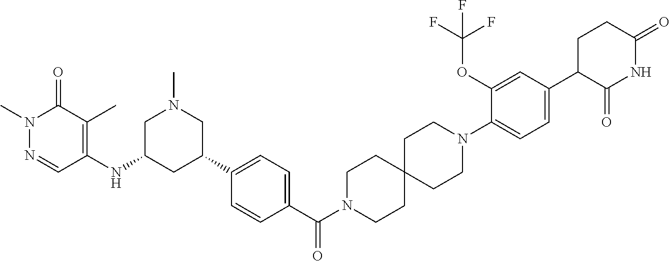 [SMILES: Cc1c(N[C@H]2C[C@@H](c3ccc(C(=O)N4CCC5(CC4)CCN(c4ccc(C6CCC(=O)NC6=O)cc4OC(F)(F)F)CC5)cc3)CN(C)C2)cnn(C)c1=O] |
| 269b | 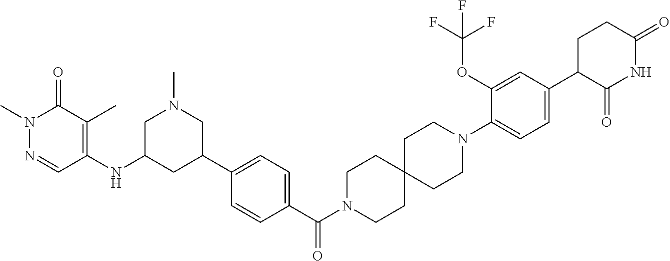 [SMILES: Cc1c(NC2CC(c3ccc(C(=O)N4CCC5(CC4)CCN(c4ccc(C6CCC(=O)NC6=O)cc4OC(F)(F)F)CC5)cc3)CN(C)C2)cnn(C)c1=O] |
| 270 | 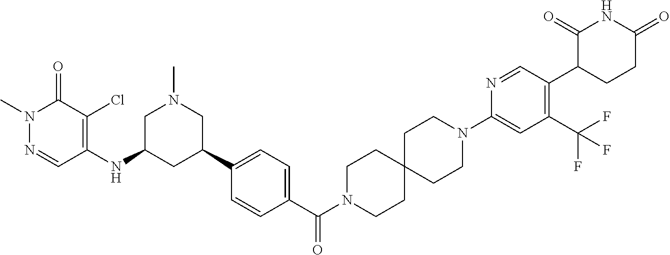 [SMILES: CN1C[C@H](Nc2cnn(C)c(=O)c2Cl)C[C@H](c2ccc(C(=O)N3CCC4(CC3)CCN(c3cc(C(F)(F)F)c(C5CCC(=O)NC5=O)cn3)CC4)cc2)C1] |
| 270a | 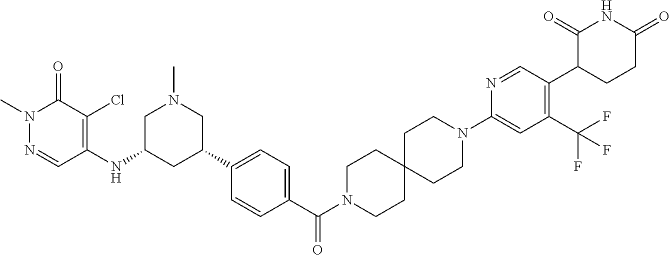 [SMILES: CN1C[C@@H](Nc2cnn(C)c(=O)c2Cl)C[C@@H](c2ccc(C(=O)N3CCC4(CC3)CCN(c3cc(C(F)(F)F)c(C5CCC(=O)NC5=O)cn3)CC4)cc2)C1] |
| 270b | 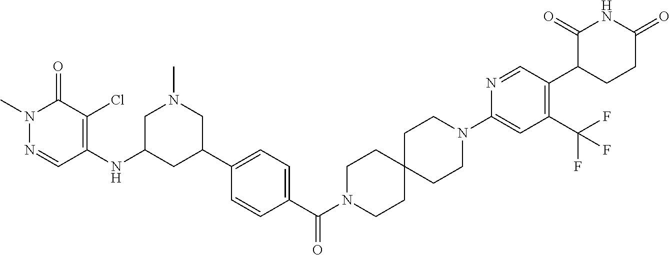 [SMILES: CN1CC(Nc2cnn(C)c(=O)c2Cl)CC(c2ccc(C(=O)N3CCC4(CC3)CCN(c3cc(C(F)(F)F)c(C5CCC(=O)NC5=O)cn3)CC4)cc2)C1] |
| 271 | 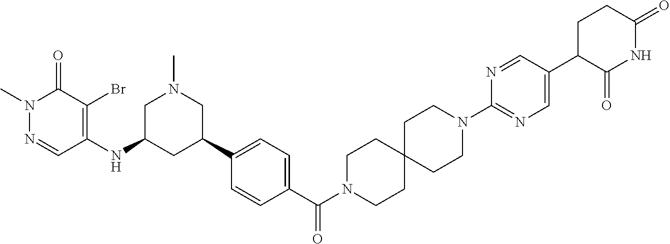 [SMILES: CN1C[C@H](Nc2cnn(C)c(=O)c2Br)C[C@H](c2ccc(C(=O)N3CCC4(CC3)CCN(c3ncc(C5CCC(=O)NC5=O)cn3)CC4)cc2)C1] |
| 271a | 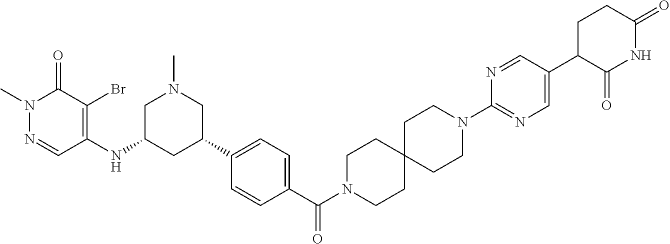 [SMILES: CN1C[C@@H](Nc2cnn(C)c(=O)c2Br)C[C@@H](c2ccc(C(=O)N3CCC4(CC3)CCN(c3ncc(C5CCC(=O)NC5=O)cn3)CC4)cc2)C1] |
| 271b | 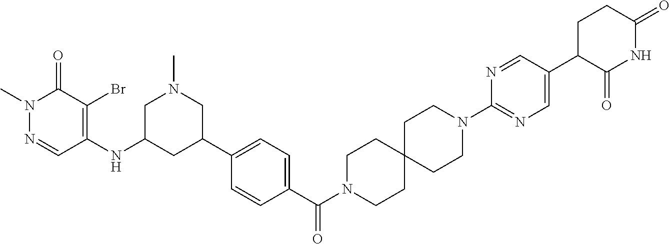 [SMILES: CN1CC(Nc2cnn(C)c(=O)c2Br)CC(c2ccc(C(=O)N3CCC4(CC3)CCN(c3ncc(C5CCC(=O)NC5=O)cn3)CC4)cc2)C1] |
| 272 | 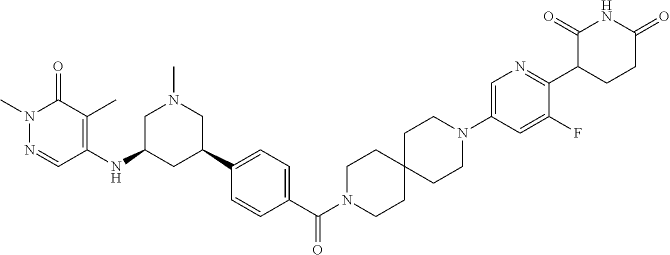 [SMILES: Cc1c(N[C@@H]2C[C@H](c3ccc(C(=O)N4CCC5(CC4)CCN(c4cnc(C6CCC(=O)NC6=O)c(F)c4)CC5)cc3)CN(C)C2)cnn(C)c1=O] |
| 272a | 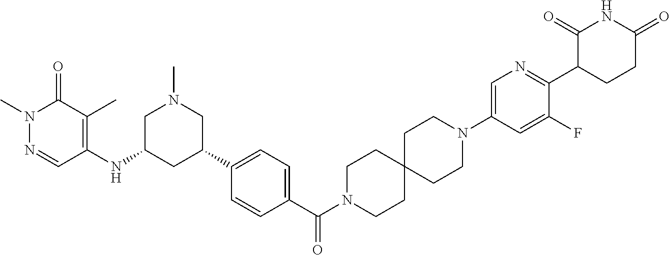 [SMILES: Cc1c(N[C@H]2C[C@@H](c3ccc(C(=O)N4CCC5(CC4)CCN(c4cnc(C6CCC(=O)NC6=O)c(F)c4)CC5)cc3)CN(C)C2)cnn(C)c1=O] |
| 272b | 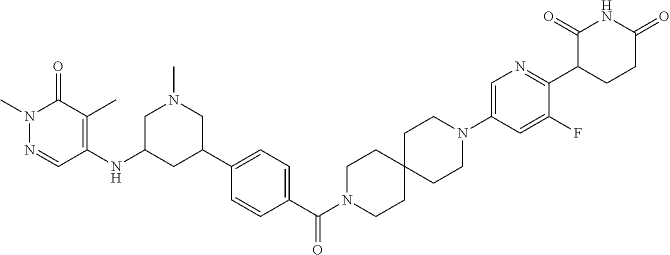 [SMILES: Cc1c(NC2CC(c3ccc(C(=O)N4CCC5(CC4)CCN(c4cnc(C6CCC(=O)NC6=O)c(F)c4)CC5)cc3)CN(C)C2)cnn(C)c1=O] |
| 273 | 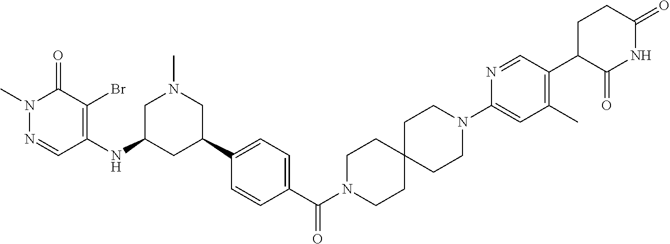 [SMILES: Cc1cc(N2CCC3(CCN(C(=O)c4ccc([C@H]5C[C@@H](Nc6cnn(C)c(=O)c6Br)CN(C)C5)cc4)CC3)CC2)ncc1C1CCC(=O)NC1=O] |
| 273a | 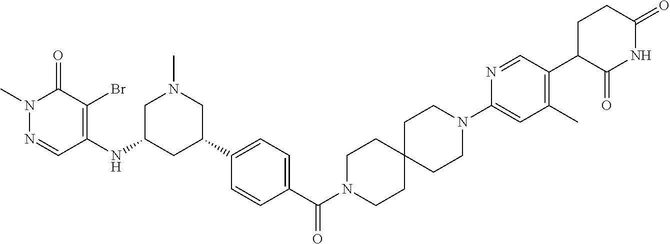 [SMILES: Cc1cc(N2CCC3(CCN(C(=O)c4ccc([C@@H]5C[C@H](Nc6cnn(C)c(=O)c6Br)CN(C)C5)cc4)CC3)CC2)ncc1C1CCC(=O)NC1=O] |
| 273b | 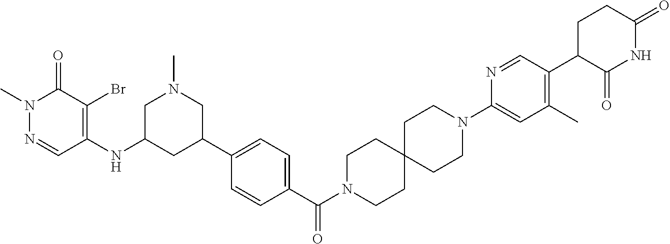 [SMILES: Cc1cc(N2CCC3(CCN(C(=O)c4ccc(C5CC(Nc6cnn(C)c(=O)c6Br)CN(C)C5)cc4)CC3)CC2)ncc1C1CCC(=O)NC1=O] |
| 274 | 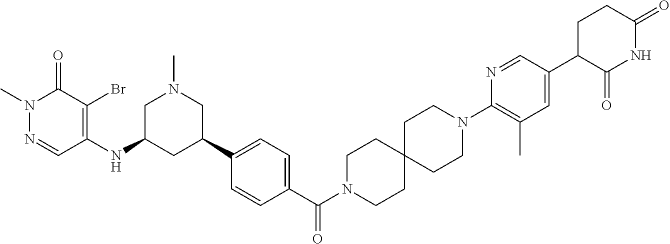 [SMILES: Cc1cc(C2CCC(=O)NC2=O)cnc1N1CCC2(CCN(C(=O)c3ccc([C@H]4C[C@@H](Nc5cnn(C)c(=O)c5Br)CN(C)C4)cc3)CC2)CC1] |
| 274a | 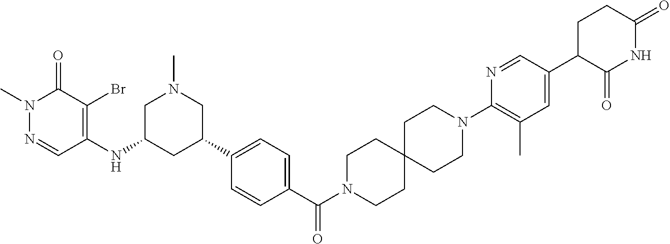 [SMILES: Cc1cc(C2CCC(=O)NC2=O)cnc1N1CCC2(CCN(C(=O)c3ccc([C@@H]4C[C@H](Nc5cnn(C)c(=O)c5Br)CN(C)C4)cc3)CC2)CC1] |
| 274b | 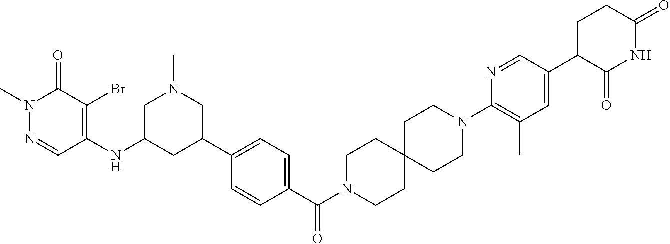 [SMILES: Cc1cc(C2CCC(=O)NC2=O)cnc1N1CCC2(CCN(C(=O)c3ccc(C4CC(Nc5cnn(C)c(=O)c5Br)CN(C)C4)cc3)CC2)CC1] |
| 275 | 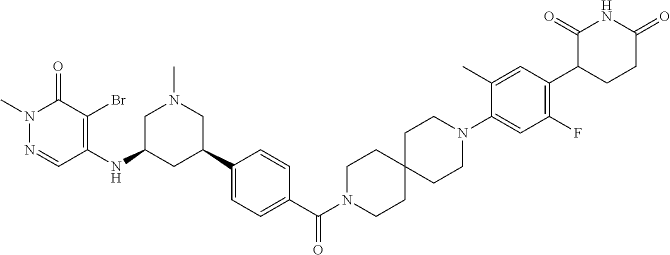 [SMILES: Cc1cc(C2CCC(=O)NC2=O)c(F)cc1N1CCC2(CCN(C(=O)c3ccc([C@H]4C[C@@H](Nc5cnn(C)c(=O)c5Br)CN(C)C4)cc3)CC2)CC1] |
| 275a | 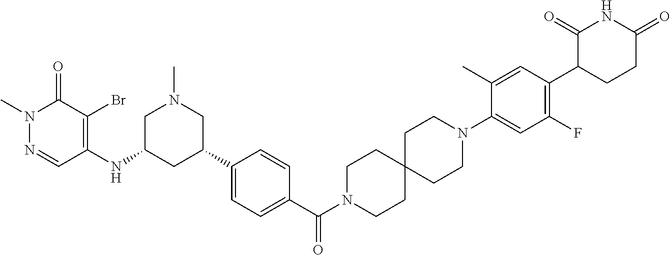 [SMILES: Cc1cc(C2CCC(=O)NC2=O)c(F)cc1N1CCC2(CCN(C(=O)c3ccc([C@@H]4C[C@H](Nc5cnn(C)c(=O)c5Br)CN(C)C4)cc3)CC2)CC1] |
| 275b | 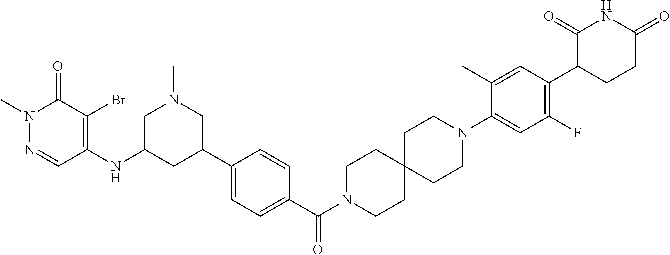 [SMILES: Cc1cc(C2CCC(=O)NC2=O)c(F)cc1N1CCC2(CCN(C(=O)c3ccc(C4CC(Nc5cnn(C)c(=O)c5Br)CN(C)C4)cc3)CC2)CC1] |
| 276 | 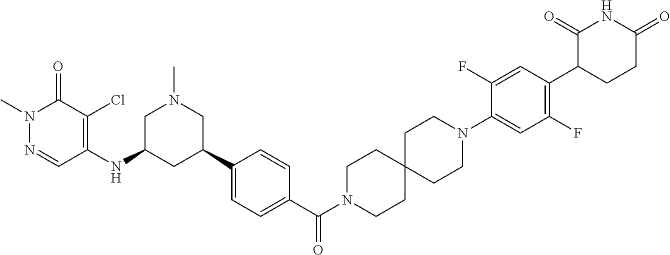 [SMILES: CN1C[C@H](Nc2cnn(C)c(=O)c2Cl)C[C@H](c2ccc(C(=O)N3CCC4(CC3)CCN(c3cc(F)c(C5CCC(=O)NC5=O)cc3F)CC4)cc2)C1] |
| 276a | 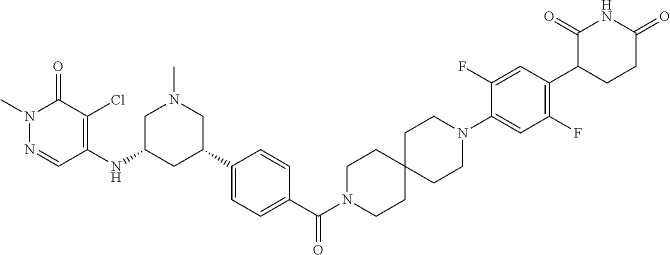 [SMILES: CN1C[C@@H](Nc2cnn(C)c(=O)c2Cl)C[C@@H](c2ccc(C(=O)N3CCC4(CC3)CCN(c3cc(F)c(C5CCC(=O)NC5=O)cc3F)CC4)cc2)C1] |
| 276b | 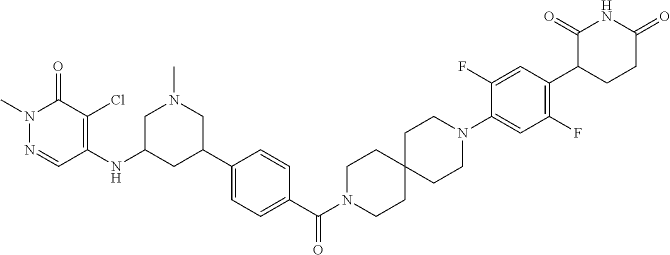 [SMILES: CN1CC(Nc2cnn(C)c(=O)c2Cl)CC(c2ccc(C(=O)N3CCC4(CC3)CCN(c3cc(F)c(C5CCC(=O)NC5=O)cc3F)CC4)cc2)C1] |
| 277 | 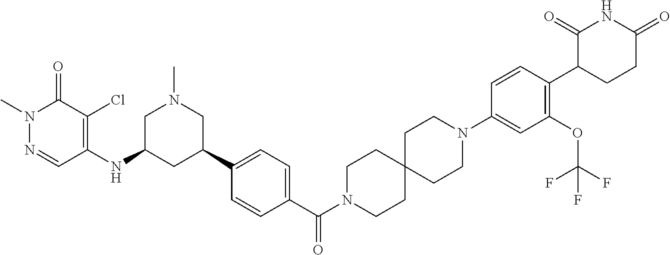 [SMILES: CN1C[C@H](Nc2cnn(C)c(=O)c2Cl)C[C@H](c2ccc(C(=O)N3CCC4(CC3)CCN(c3ccc(C5CCC(=O)NC5=O)c(OC(F)(F)F)c3)CC4)cc2)C1] |
| 277a | 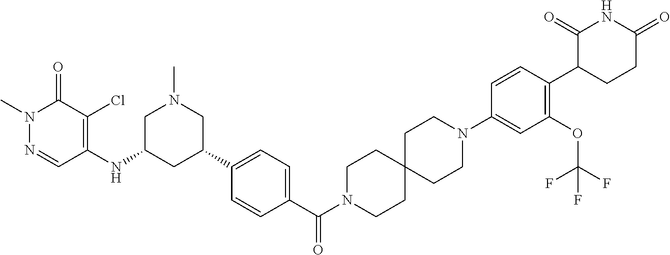 [SMILES: CN1C[C@@H](Nc2cnn(C)c(=O)c2Cl)C[C@@H](c2ccc(C(=O)N3CCC4(CC3)CCN(c3ccc(C5CCC(=O)NC5=O)c(OC(F)(F)F)c3)CC4)cc2)C1] |
| 277b | 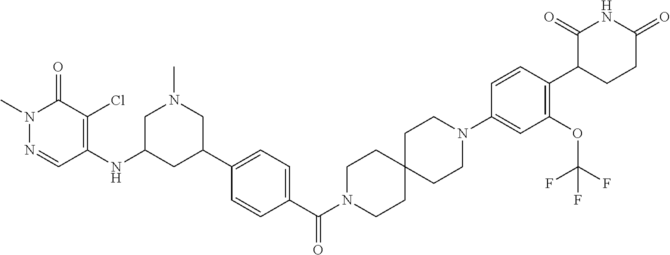 [SMILES: CN1CC(Nc2cnn(C)c(=O)c2Cl)CC(c2ccc(C(=O)N3CCC4(CC3)CCN(c3ccc(C5CCC(=O)NC5=O)c(OC(F)(F)F)c3)CC4)cc2)C1] |
| 278 | 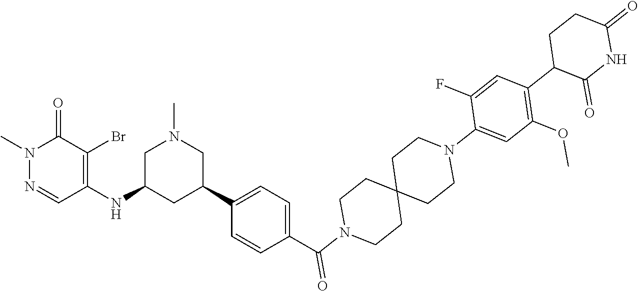 [SMILES: COc1cc(N2CCC3(CCN(C(=O)c4ccc([C@H]5C[C@@H](Nc6cnn(C)c(=O)c6Br)CN(C)C5)cc4)CC3)CC2)c(F)cc1C1CCC(=O)NC1=O] |
| 278a | 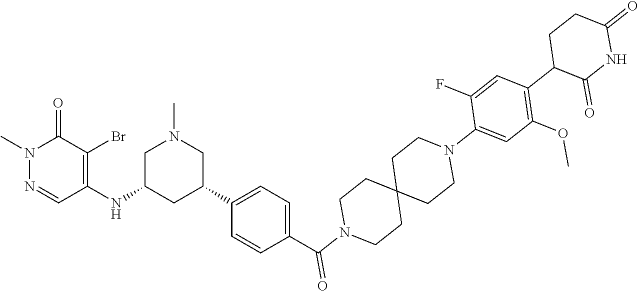 [SMILES: COc1cc(N2CCC3(CCN(C(=O)c4ccc([C@@H]5C[C@H](Nc6cnn(C)c(=O)c6Br)CN(C)C5)cc4)CC3)CC2)c(F)cc1C1CCC(=O)NC1=O] |
| 278b | 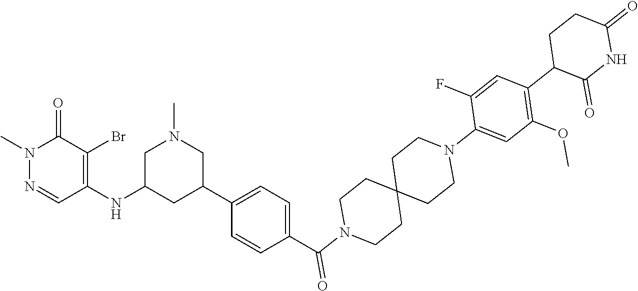 [SMILES: COc1cc(N2CCC3(CCN(C(=O)c4ccc(C5CC(Nc6cnn(C)c(=O)c6Br)CN(C)C5)cc4)CC3)CC2)c(F)cc1C1CCC(=O)NC1=O] |
| 279 | 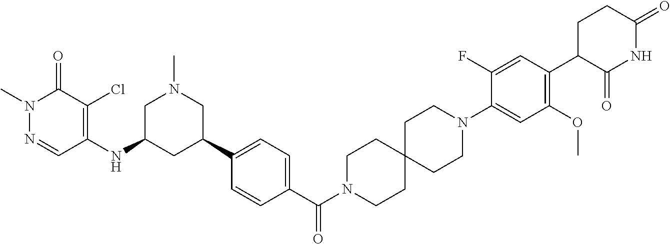 [SMILES: COc1cc(N2CCC3(CCN(C(=O)c4ccc([C@H]5C[C@@H](Nc6cnn(C)c(=O)c6Cl)CN(C)C5)cc4)CC3)CC2)c(F)cc1C1CCC(=O)NC1=O] |
| 279a | 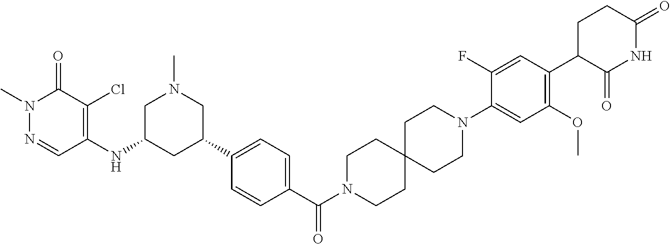 [SMILES: COc1cc(N2CCC3(CCN(C(=O)c4ccc([C@@H]5C[C@H](Nc6cnn(C)c(=O)c6Cl)CN(C)C5)cc4)CC3)CC2)c(F)cc1C1CCC(=O)NC1=O] |
| 279b | 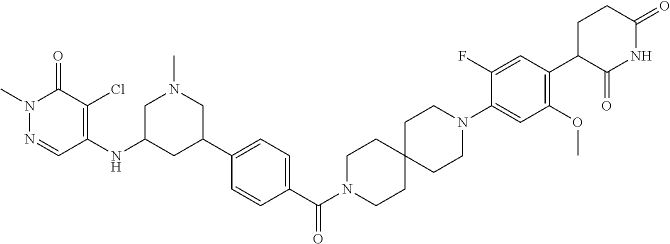 [SMILES: COc1cc(N2CCC3(CCN(C(=O)c4ccc(C5CC(Nc6cnn(C)c(=O)c6Cl)CN(C)C5)cc4)CC3)CC2)c(F)cc1C1CCC(=O)NC1=O] |
| 280 | 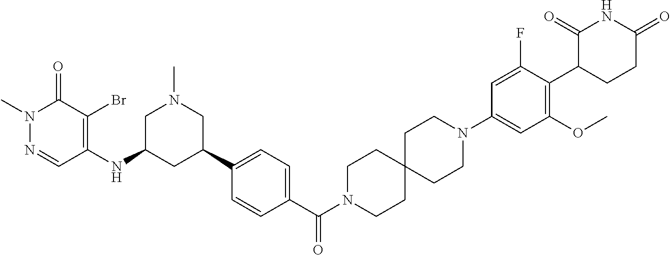 [SMILES: COc1cc(N2CCC3(CCN(C(=O)c4ccc([C@H]5C[C@@H](Nc6cnn(C)c(=O)c6Br)CN(C)C5)cc4)CC3)CC2)cc(F)c1C1CCC(=O)NC1=O] |
| 280a | 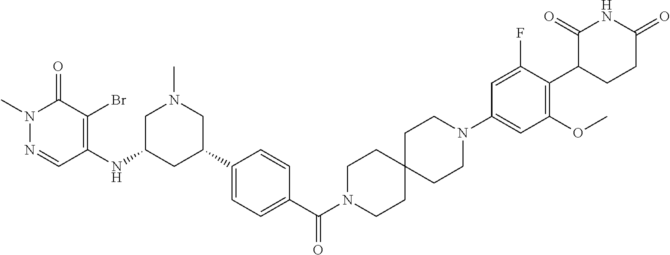 [SMILES: COc1cc(N2CCC3(CCN(C(=O)c4ccc([C@@H]5C[C@H](Nc6cnn(C)c(=O)c6Br)CN(C)C5)cc4)CC3)CC2)cc(F)c1C1CCC(=O)NC1=O] |
| 280b | 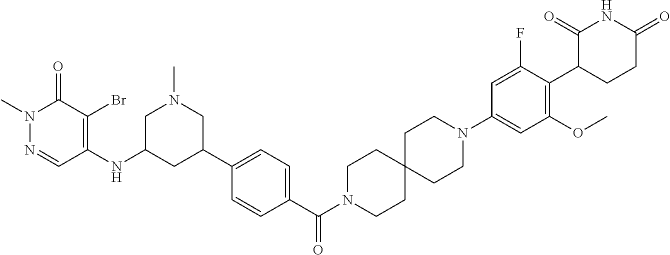 [SMILES: COc1cc(N2CCC3(CCN(C(=O)c4ccc(C5CC(Nc6cnn(C)c(=O)c6Br)CN(C)C5)cc4)CC3)CC2)cc(F)c1C1CCC(=O)NC1=O] |
| 281 | 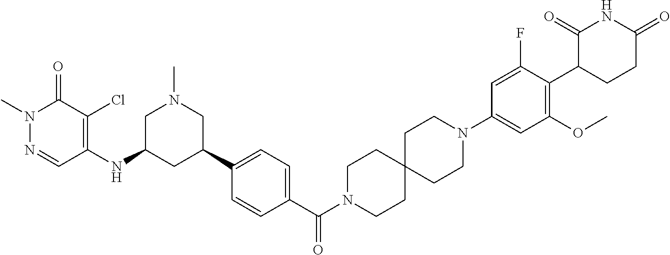 [SMILES: COc1cc(N2CCC3(CCN(C(=O)c4ccc([C@H]5C[C@@H](Nc6cnn(C)c(=O)c6Cl)CN(C)C5)cc4)CC3)CC2)cc(F)c1C1CCC(=O)NC1=O] |
| 281a | 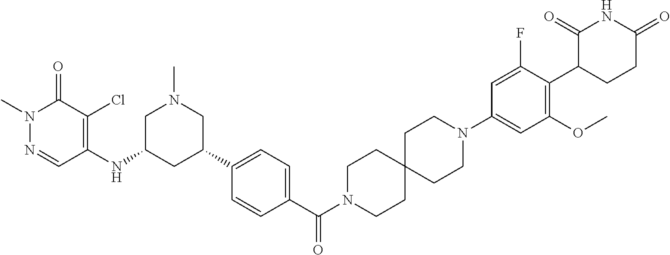 [SMILES: COc1cc(N2CCC3(CCN(C(=O)c4ccc([C@@H]5C[C@H](Nc6cnn(C)c(=O)c6Cl)CN(C)C5)cc4)CC3)CC2)cc(F)c1C1CCC(=O)NC1=O] |
| 281b | 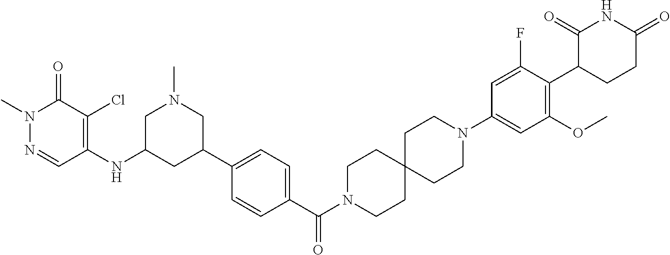 [SMILES: COc1cc(N2CCC3(CCN(C(=O)c4ccc(C5CC(Nc6cnn(C)c(=O)c6Cl)CN(C)C5)cc4)CC3)CC2)cc(F)c1C1CCC(=O)NC1=O] |
| 282 | 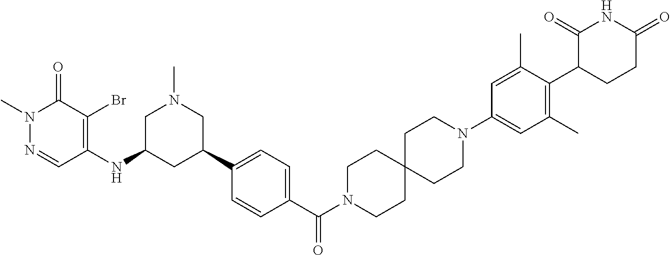 [SMILES: Cc1cc(N2CCC3(CCN(C(=O)c4ccc([C@H]5C[C@@H](Nc6cnn(C)c(=O)c6Br)CN(C)C5)cc4)CC3)CC2)cc(C)c1C1CCC(=O)NC1=O] |
| 282a | 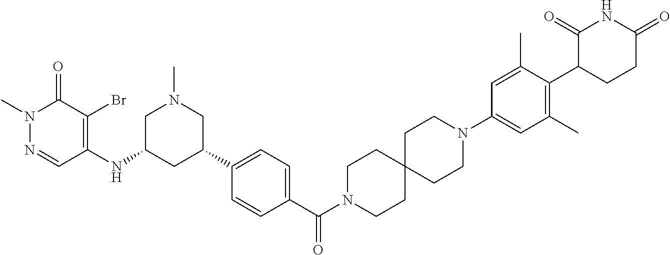 [SMILES: Cc1cc(N2CCC3(CCN(C(=O)c4ccc([C@@H]5C[C@H](Nc6cnn(C)c(=O)c6Br)CN(C)C5)cc4)CC3)CC2)cc(C)c1C1CCC(=O)NC1=O] |
| 282b | 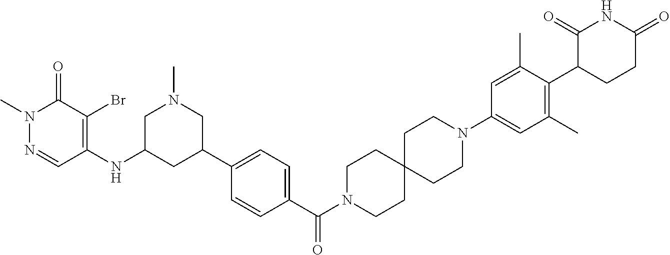 [SMILES: Cc1cc(N2CCC3(CCN(C(=O)c4ccc(C5CC(Nc6cnn(C)c(=O)c6Br)CN(C)C5)cc4)CC3)CC2)cc(C)c1C1CCC(=O)NC1=O] |
| 283 | 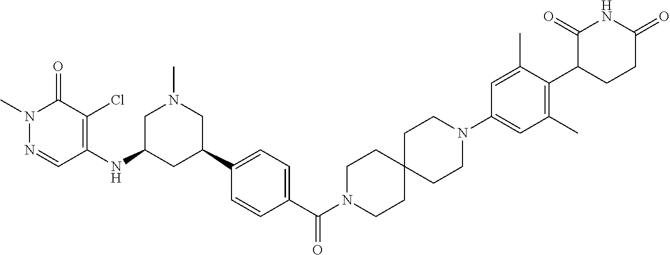 [SMILES: Cc1cc(N2CCC3(CCN(C(=O)c4ccc([C@H]5C[C@@H](Nc6cnn(C)c(=O)c6Cl)CN(C)C5)cc4)CC3)CC2)cc(C)c1C1CCC(=O)NC1=O] |
| 283a | 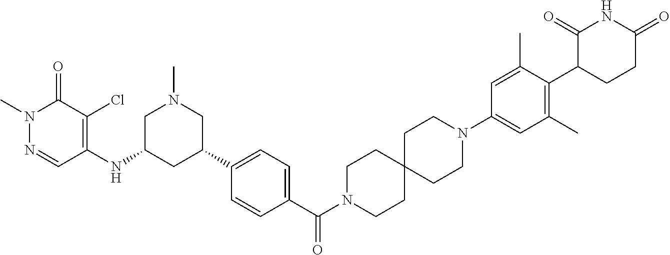 [SMILES: Cc1cc(N2CCC3(CCN(C(=O)c4ccc([C@@H]5C[C@H](Nc6cnn(C)c(=O)c6Cl)CN(C)C5)cc4)CC3)CC2)cc(C)c1C1CCC(=O)NC1=O] |
| 283b | 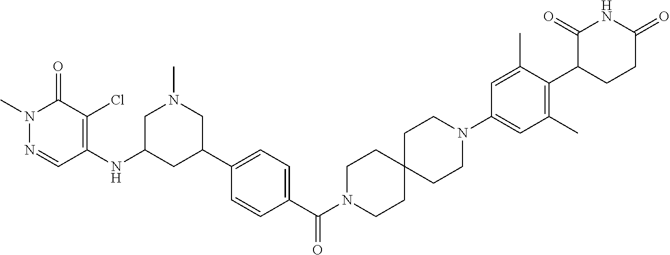 [SMILES: Cc1cc(N2CCC3(CCN(C(=O)c4ccc(C5CC(Nc6cnn(C)c(=O)c6Cl)CN(C)C5)cc4)CC3)CC2)cc(C)c1C1CCC(=O)NC1=O] |
| 284 | 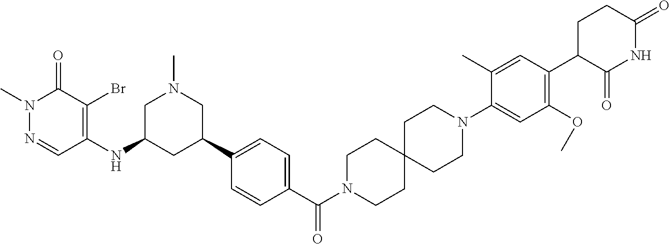 [SMILES: COc1cc(N2CCC3(CCN(C(=O)c4ccc([C@H]5C[C@@H](Nc6cnn(C)c(=O)c6Br)CN(C)C5)cc4)CC3)CC2)c(C)cc1C1CCC(=O)NC1=O] |
| 284a | 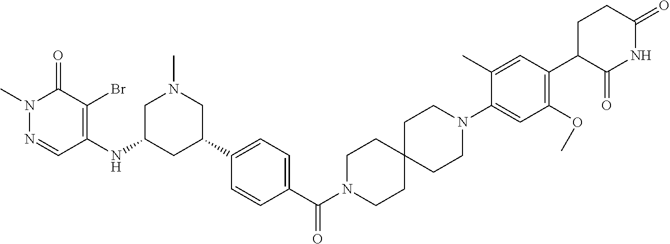 [SMILES: COc1cc(N2CCC3(CCN(C(=O)c4ccc([C@@H]5C[C@H](Nc6cnn(C)c(=O)c6Br)CN(C)C5)cc4)CC3)CC2)c(C)cc1C1CCC(=O)NC1=O] |
| 284b | 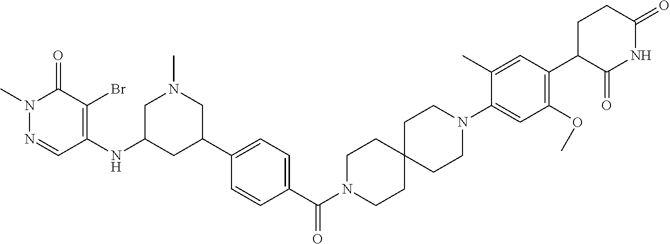 [SMILES: COc1cc(N2CCC3(CCN(C(=O)c4ccc(C5CC(Nc6cnn(C)c(=O)c6Br)CN(C)C5)cc4)CC3)CC2)c(C)cc1C1CCC(=O)NC1=O] |
| 285 | 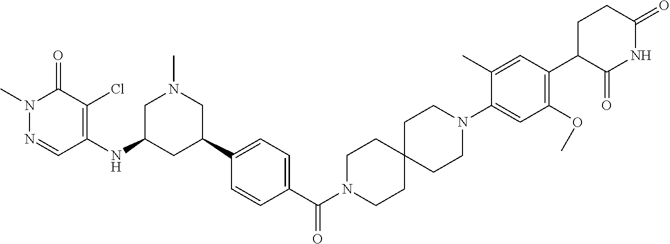 [SMILES: COc1cc(N2CCC3(CCN(C(=O)c4ccc([C@H]5C[C@@H](Nc6cnn(C)c(=O)c6Cl)CN(C)C5)cc4)CC3)CC2)c(C)cc1C1CCC(=O)NC1=O] |
| 285a | 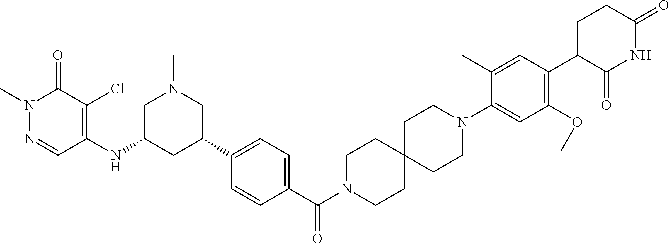 [SMILES: COc1cc(N2CCC3(CCN(C(=O)c4ccc([C@@H]5C[C@H](Nc6cnn(C)c(=O)c6Cl)CN(C)C5)cc4)CC3)CC2)c(C)cc1C1CCC(=O)NC1=O] |
| 285b | 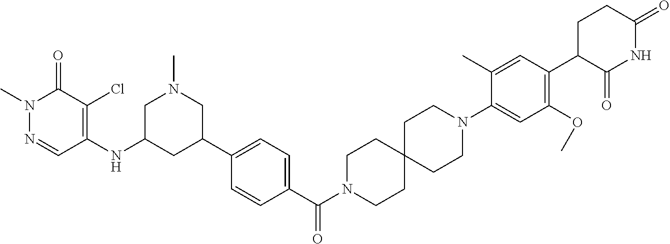 [SMILES: COc1cc(N2CCC3(CCN(C(=O)c4ccc(C5CC(Nc6cnn(C)c(=O)c6Cl)CN(C)C5)cc4)CC3)CC2)c(C)cc1C1CCC(=O)NC1=O] |
| 286 | 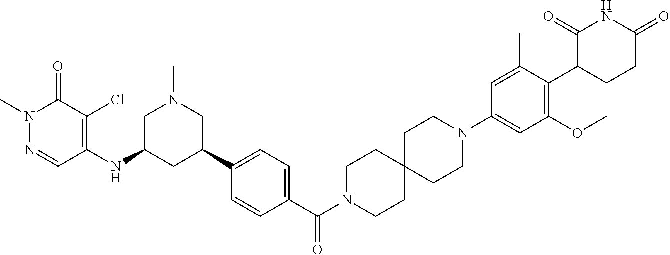 [SMILES: COc1cc(N2CCC3(CCN(C(=O)c4ccc([C@H]5C[C@@H](Nc6cnn(C)c(=O)c6Cl)CN(C)C5)cc4)CC3)CC2)cc(C)c1C1CCC(=O)NC1=O] |
| 286a | 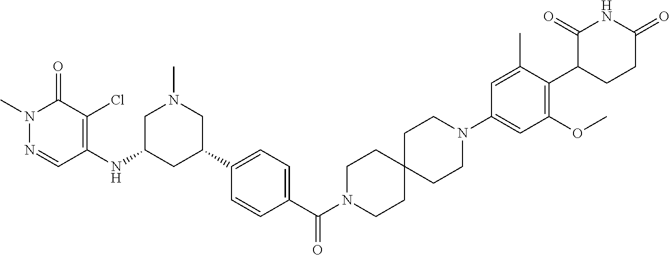 [SMILES: COc1cc(N2CCC3(CCN(C(=O)c4ccc([C@@H]5C[C@H](Nc6cnn(C)c(=O)c6Cl)CN(C)C5)cc4)CC3)CC2)cc(C)c1C1CCC(=O)NC1=O] |
| 286b | 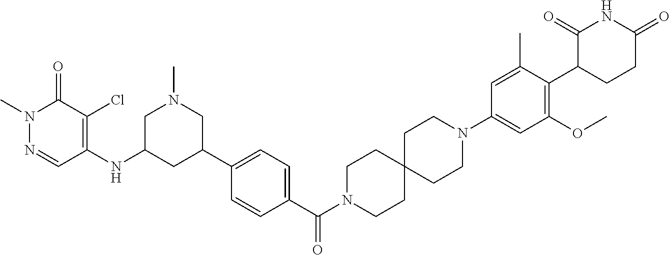 [SMILES: COc1cc(N2CCC3(CCN(C(=O)c4ccc(C5CC(Nc6cnn(C)c(=O)c6Cl)CN(C)C5)cc4)CC3)CC2)cc(C)c1C1CCC(=O)NC1=O] |
| 287 | 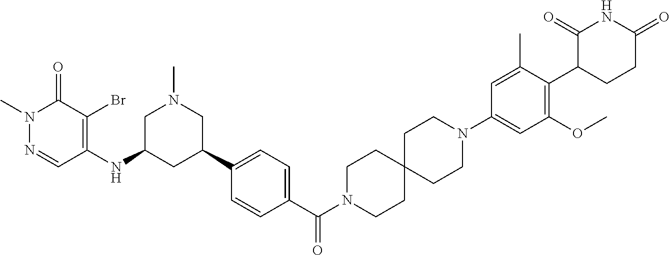 [SMILES: COc1cc(N2CCC3(CCN(C(=O)c4ccc([C@H]5C[C@@H](Nc6cnn(C)c(=O)c6Br)CN(C)C5)cc4)CC3)CC2)cc(C)c1C1CCC(=O)NC1=O] |
| 287a | 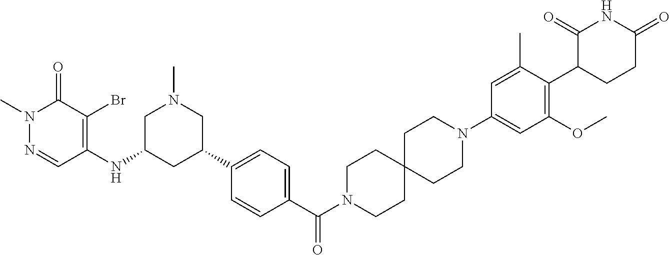 [SMILES: COc1cc(N2CCC3(CCN(C(=O)c4ccc([C@@H]5C[C@H](Nc6cnn(C)c(=O)c6Br)CN(C)C5)cc4)CC3)CC2)cc(C)c1C1CCC(=O)NC1=O] |
| 287b | 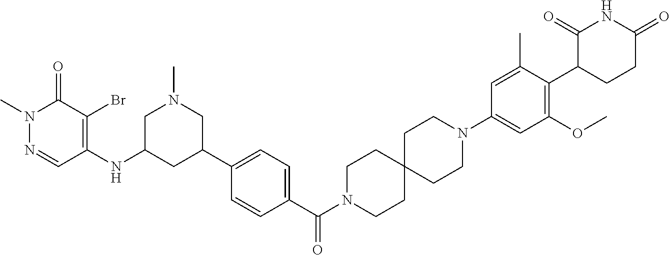 [SMILES: COc1cc(N2CCC3(CCN(C(=O)c4ccc(C5CC(Nc6cnn(C)c(=O)c6Br)CN(C)C5)cc4)CC3)CC2)cc(C)c1C1CCC(=O)NC1=O] |
| 288 | 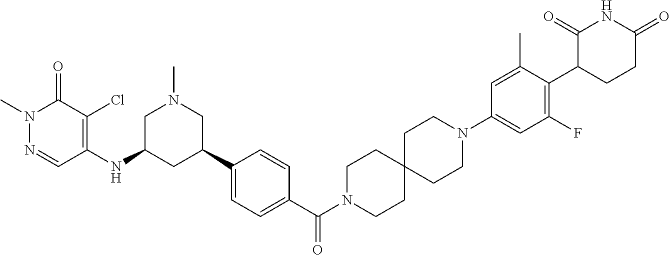 [SMILES: Cc1cc(N2CCC3(CCN(C(=O)c4ccc([C@H]5C[C@@H](Nc6cnn(C)c(=O)c6Cl)CN(C)C5)cc4)CC3)CC2)cc(F)c1C1CCC(=O)NC1=O] |
| 288a | 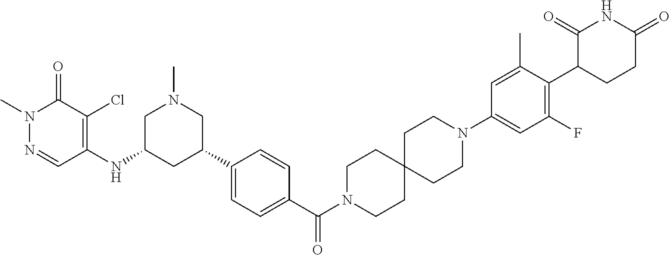 [SMILES: Cc1cc(N2CCC3(CCN(C(=O)c4ccc([C@@H]5C[C@H](Nc6cnn(C)c(=O)c6Cl)CN(C)C5)cc4)CC3)CC2)cc(F)c1C1CCC(=O)NC1=O] |
| 288b | 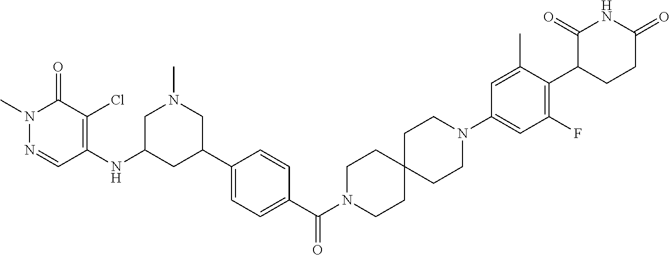 [SMILES: Cc1cc(N2CCC3(CCN(C(=O)c4ccc(C5CC(Nc6cnn(C)c(=O)c6Cl)CN(C)C5)cc4)CC3)CC2)cc(F)c1C1CCC(=O)NC1=O] |
| 289 | 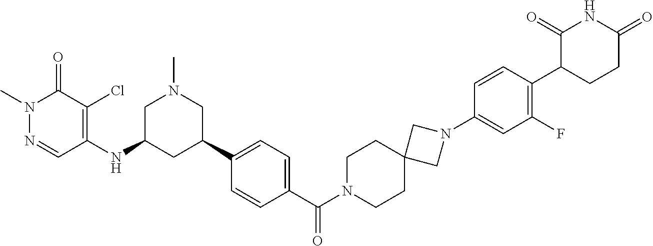 [SMILES: CN1C[C@H](Nc2cnn(C)c(=O)c2Cl)C[C@H](c2ccc(C(=O)N3CCC4(CC3)CN(c3ccc(C5CCC(=O)NC5=O)c(F)c3)C4)cc2)C1] |
| 289a | 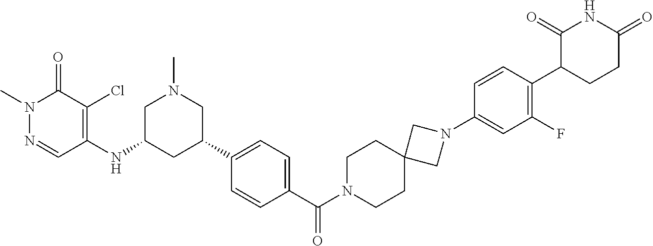 [SMILES: CN1C[C@@H](Nc2cnn(C)c(=O)c2Cl)C[C@@H](c2ccc(C(=O)N3CCC4(CC3)CN(c3ccc(C5CCC(=O)NC5=O)c(F)c3)C4)cc2)C1] |
| 289b | 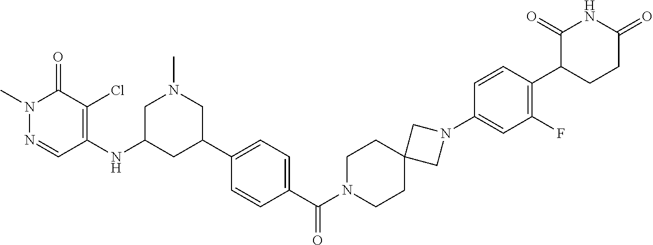 [SMILES: CN1CC(Nc2cnn(C)c(=O)c2Cl)CC(c2ccc(C(=O)N3CCC4(CC3)CN(c3ccc(C5CCC(=O)NC5=O)c(F)c3)C4)cc2)C1] |
| 290 | 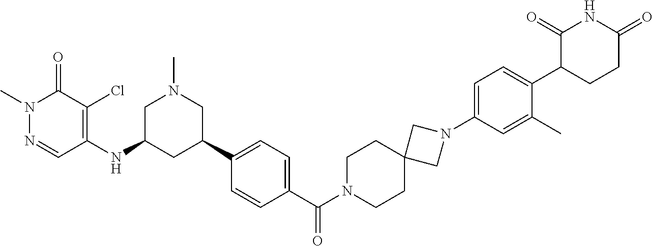 [SMILES: Cc1cc(N2CC3(CCN(C(=O)c4ccc([C@H]5C[C@@H](Nc6cnn(C)c(=O)c6Cl)CN(C)C5)cc4)CC3)C2)ccc1C1CCC(=O)NC1=O] |
| 290a | 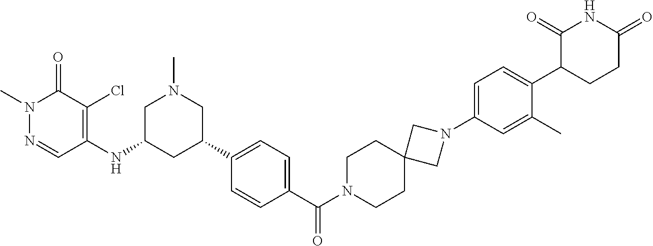 [SMILES: Cc1cc(N2CC3(CCN(C(=O)c4ccc([C@@H]5C[C@H](Nc6cnn(C)c(=O)c6Cl)CN(C)C5)cc4)CC3)C2)ccc1C1CCC(=O)NC1=O] |
| 290b | 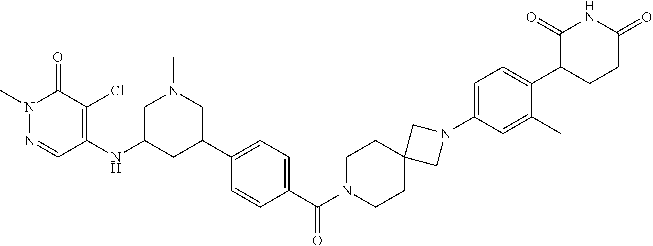 [SMILES: Cc1cc(N2CC3(CCN(C(=O)c4ccc(C5CC(Nc6cnn(C)c(=O)c6Cl)CN(C)C5)cc4)CC3)C2)ccc1C1CCC(=O)NC1=O] |
| 291 | 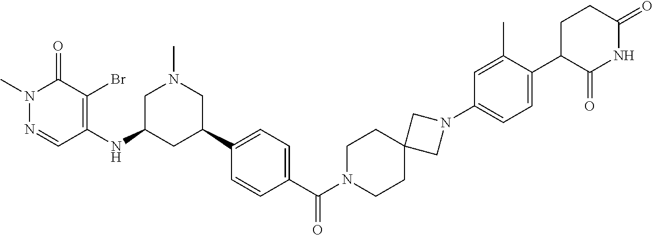 [SMILES: Cc1cc(N2CC3(CCN(C(=O)c4ccc([C@H]5C[C@@H](Nc6cnn(C)c(=O)c6Br)CN(C)C5)cc4)CC3)C2)ccc1C1CCC(=O)NC1=O] |
| 291a | 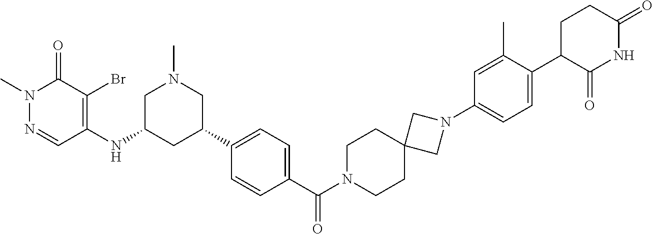 [SMILES: Cc1cc(N2CC3(CCN(C(=O)c4ccc([C@@H]5C[C@H](Nc6cnn(C)c(=O)c6Br)CN(C)C5)cc4)CC3)C2)ccc1C1CCC(=O)NC1=O] |
| 291b | 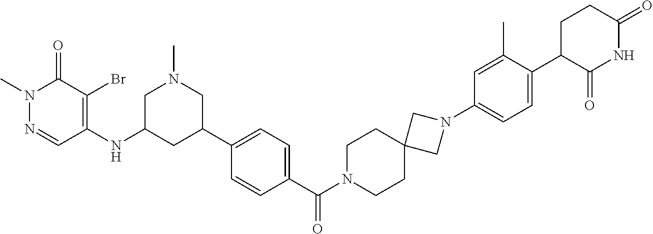 [SMILES: Cc1cc(N2CC3(CCN(C(=O)c4ccc(C5CC(Nc6cnn(C)c(=O)c6Br)CN(C)C5)cc4)CC3)C2)ccc1C1CCC(=O)NC1=O] |
| 292 | 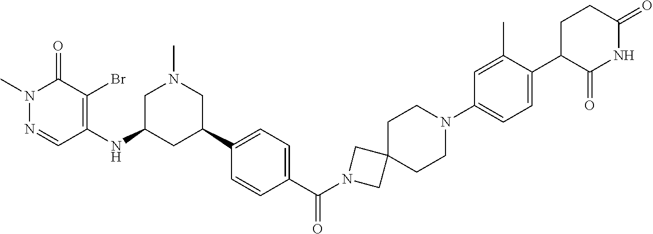 [SMILES: Cc1cc(N2CCC3(CC2)CN(C(=O)c2ccc([C@H]4C[C@@H](Nc5cnn(C)c(=O)c5Br)CN(C)C4)cc2)C3)ccc1C1CCC(=O)NC1=O] |
| 292a | 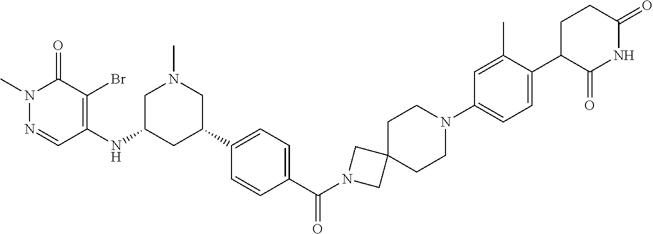 [SMILES: Cc1cc(N2CCC3(CC2)CN(C(=O)c2ccc([C@@H]4C[C@H](Nc5cnn(C)c(=O)c5Br)CN(C)C4)cc2)C3)ccc1C1CCC(=O)NC1=O] |
| 292b | 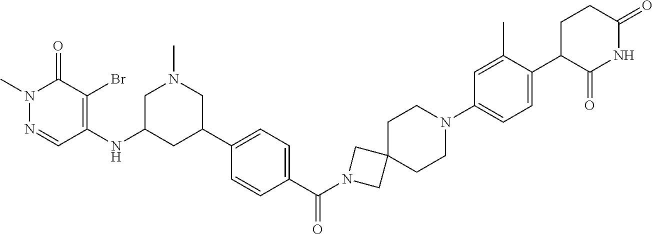 [SMILES: Cc1cc(N2CCC3(CC2)CN(C(=O)c2ccc(C4CC(Nc5cnn(C)c(=O)c5Br)CN(C)C4)cc2)C3)ccc1C1CCC(=O)NC1=O] |
| 293 | 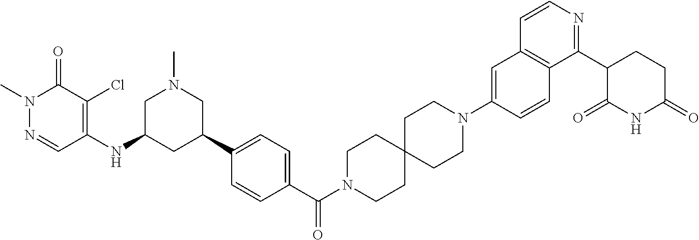 [SMILES: CN1C[C@H](Nc2cnn(C)c(=O)c2Cl)C[C@H](c2ccc(C(=O)N3CCC4(CC3)CCN(c3ccc5c(C6CCC(=O)NC6=O)nccc5c3)CC4)cc2)C1] |
| 293a | 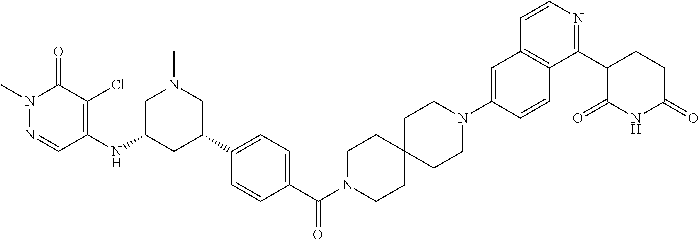 [SMILES: CN1C[C@@H](Nc2cnn(C)c(=O)c2Cl)C[C@@H](c2ccc(C(=O)N3CCC4(CC3)CCN(c3ccc5c(C6CCC(=O)NC6=O)nccc5c3)CC4)cc2)C1] |
| 293b | 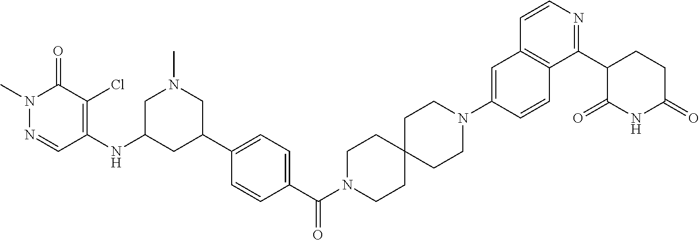 [SMILES: CN1CC(Nc2cnn(C)c(=O)c2Cl)CC(c2ccc(C(=O)N3CCC4(CC3)CCN(c3ccc5c(C6CCC(=O)NC6=O)nccc5c3)CC4)cc2)C1] |
| 294 | 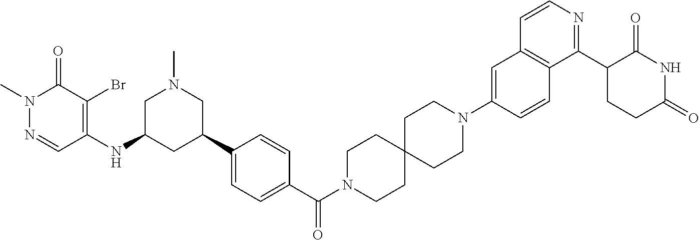 [SMILES: CN1C[C@H](Nc2cnn(C)c(=O)c2Br)C[C@H](c2ccc(C(=O)N3CCC4(CC3)CCN(c3ccc5c(C6CCC(=O)NC6=O)nccc5c3)CC4)cc2)C1] |
| 294a | 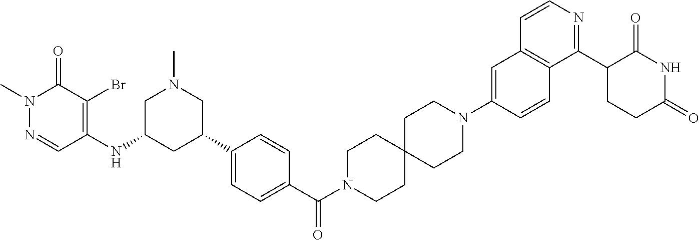 [SMILES: CN1C[C@@H](Nc2cnn(C)c(=O)c2Br)C[C@@H](c2ccc(C(=O)N3CCC4(CC3)CCN(c3ccc5c(C6CCC(=O)NC6=O)nccc5c3)CC4)cc2)C1] |
| 294b | 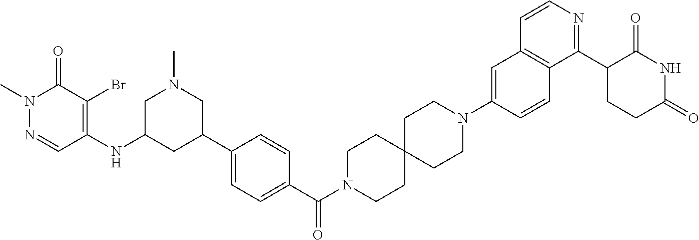 [SMILES: CN1CC(Nc2cnn(C)c(=O)c2Br)CC(c2ccc(C(=O)N3CCC4(CC3)CCN(c3ccc5c(C6CCC(=O)NC6=O)nccc5c3)CC4)cc2)C1] |
| 295 | 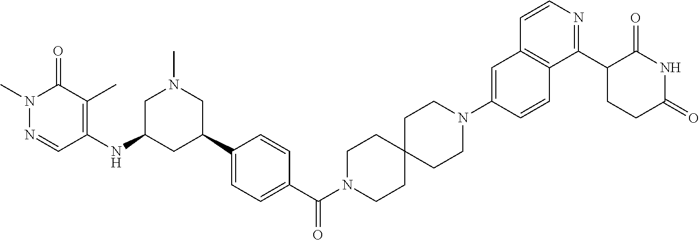 [SMILES: Cc1c(N[C@@H]2C[C@H](c3ccc(C(=O)N4CCC5(CC4)CCN(c4ccc6c(C7CCC(=O)NC7=O)nccc6c4)CC5)cc3)CN(C)C2)cnn(C)c1=O] |
| 295a | 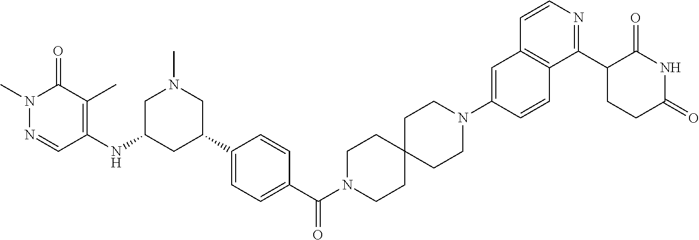 [SMILES: Cc1c(N[C@H]2C[C@@H](c3ccc(C(=O)N4CCC5(CC4)CCN(c4ccc6c(C7CCC(=O)NC7=O)nccc6c4)CC5)cc3)CN(C)C2)cnn(C)c1=O] |
| 295b | 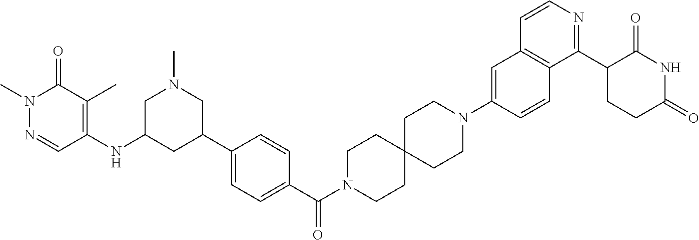 [SMILES: Cc1c(NC2CC(c3ccc(C(=O)N4CCC5(CC4)CCN(c4ccc6c(C7CCC(=O)NC7=O)nccc6c4)CC5)cc3)CN(C)C2)cnn(C)c1=O] |
| 296 | 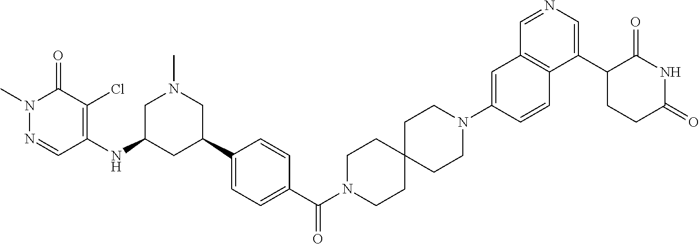 [SMILES: CN1C[C@H](Nc2cnn(C)c(=O)c2Cl)C[C@H](c2ccc(C(=O)N3CCC4(CC3)CCN(c3ccc5c(C6CCC(=O)NC6=O)cncc5c3)CC4)cc2)C1] |
| 296a | 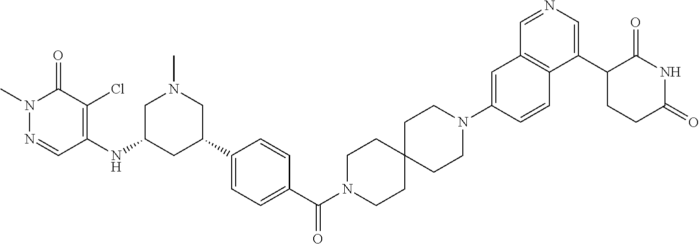 [SMILES: CN1C[C@@H](Nc2cnn(C)c(=O)c2Cl)C[C@@H](c2ccc(C(=O)N3CCC4(CC3)CCN(c3ccc5c(C6CCC(=O)NC6=O)cncc5c3)CC4)cc2)C1] |
| 296b | 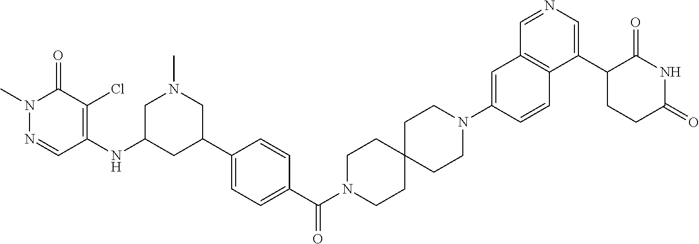 [SMILES: CN1CC(Nc2cnn(C)c(=O)c2Cl)CC(c2ccc(C(=O)N3CCC4(CC3)CCN(c3ccc5c(C6CCC(=O)NC6=O)cncc5c3)CC4)cc2)C1] |
| 297 | 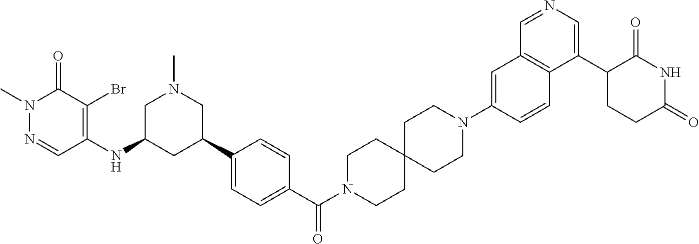 [SMILES: CN1C[C@H](Nc2cnn(C)c(=O)c2Br)C[C@H](c2ccc(C(=O)N3CCC4(CC3)CCN(c3ccc5c(C6CCC(=O)NC6=O)cncc5c3)CC4)cc2)C1] |
| 297a | 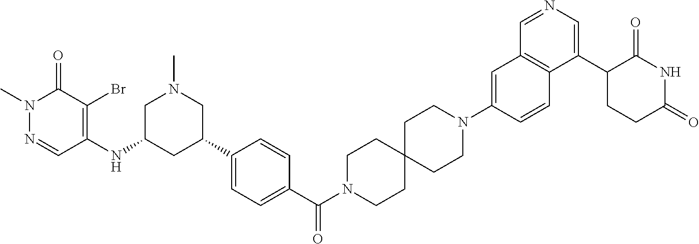 [SMILES: CN1C[C@@H](Nc2cnn(C)c(=O)c2Br)C[C@@H](c2ccc(C(=O)N3CCC4(CC3)CCN(c3ccc5c(C6CCC(=O)NC6=O)cncc5c3)CC4)cc2)C1] |
| 297b | 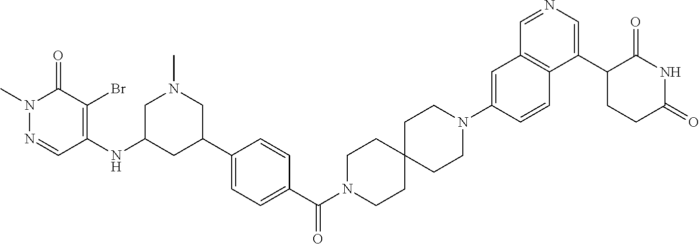 [SMILES: CN1CC(Nc2cnn(C)c(=O)c2Br)CC(c2ccc(C(=O)N3CCC4(CC3)CCN(c3ccc5c(C6CCC(=O)NC6=O)cncc5c3)CC4)cc2)C1] |
| 298 | 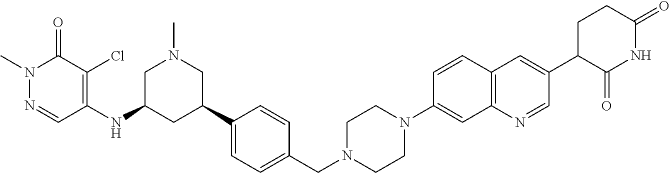 [SMILES: CN1C[C@H](Nc2cnn(C)c(=O)c2Cl)C[C@H](c2ccc(CN3CCN(c4ccc5cc(C6CCC(=O)NC6=O)cnc5c4)CC3)cc2)C1] |
| 298a | 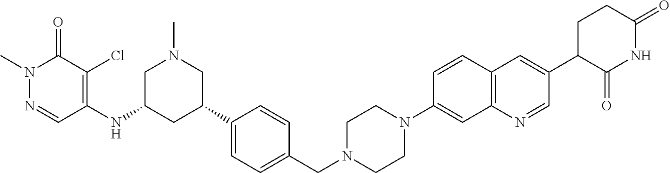 [SMILES: CN1C[C@@H](Nc2cnn(C)c(=O)c2Cl)C[C@@H](c2ccc(CN3CCN(c4ccc5cc(C6CCC(=O)NC6=O)cnc5c4)CC3)cc2)C1] |
| 298b | 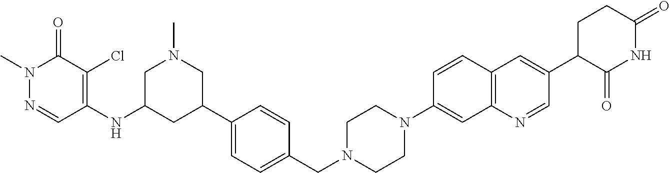 [SMILES: CN1CC(Nc2cnn(C)c(=O)c2Cl)CC(c2ccc(CN3CCN(c4ccc5cc(C6CCC(=O)NC6=O)cnc5c4)CC3)cc2)C1] |
| 299 | 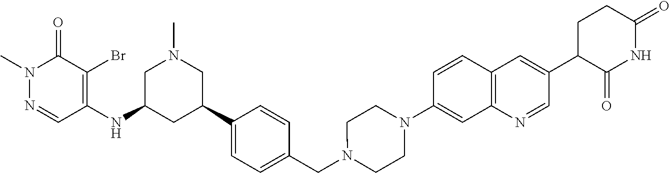 [SMILES: CN1C[C@H](Nc2cnn(C)c(=O)c2Br)C[C@H](c2ccc(CN3CCN(c4ccc5cc(C6CCC(=O)NC6=O)cnc5c4)CC3)cc2)C1] |
| 299a | 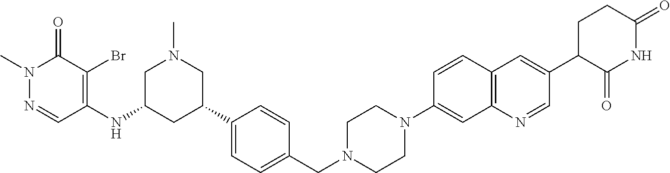 [SMILES: CN1C[C@@H](Nc2cnn(C)c(=O)c2Br)C[C@@H](c2ccc(CN3CCN(c4ccc5cc(C6CCC(=O)NC6=O)cnc5c4)CC3)cc2)C1] |
| 299b | 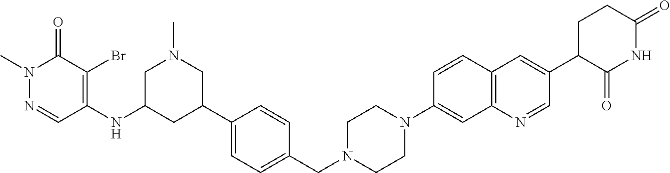 [SMILES: CN1CC(Nc2cnn(C)c(=O)c2Br)CC(c2ccc(CN3CCN(c4ccc5cc(C6CCC(=O)NC6=O)cnc5c4)CC3)cc2)C1] |
| 300 | 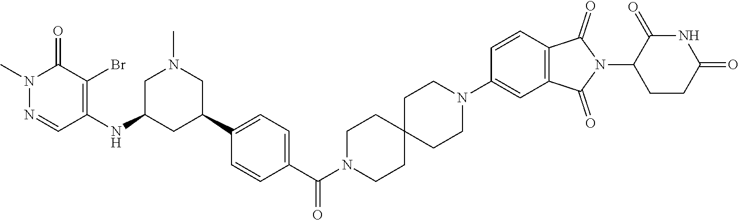 [SMILES: CN1C[C@H](Nc2cnn(C)c(=O)c2Br)C[C@H](c2ccc(C(=O)N3CCC4(CC3)CCN(c3ccc5c(c3)C(=O)N(C3CCC(=O)NC3=O)C5=O)CC4)cc2)C1] |
| 300a | 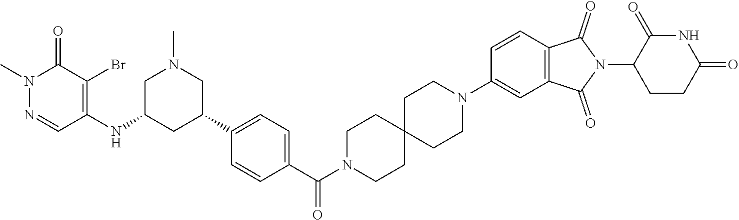 [SMILES: CN1C[C@@H](Nc2cnn(C)c(=O)c2Br)C[C@@H](c2ccc(C(=O)N3CCC4(CC3)CCN(c3ccc5c(c3)C(=O)N(C3CCC(=O)NC3=O)C5=O)CC4)cc2)C1] |
| 300b | 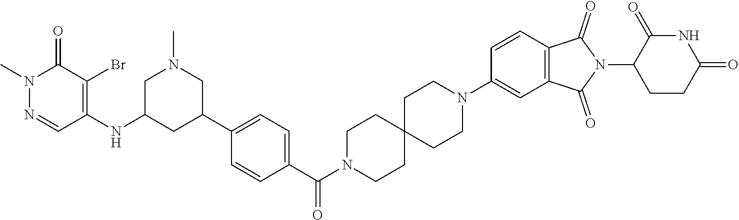 [SMILES: CN1CC(Nc2cnn(C)c(=O)c2Br)CC(c2ccc(C(=O)N3CCC4(CC3)CCN(c3ccc5c(c3)C(=O)N(C3CCC(=O)NC3=O)C5=O)CC4)cc2)C1] |
| 301 | 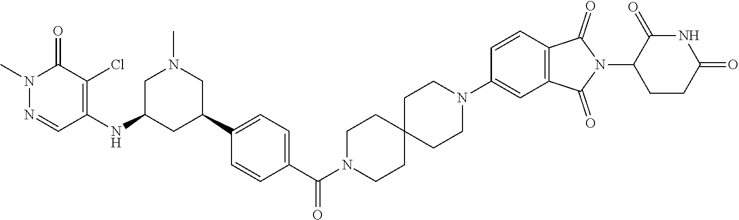 [SMILES: CN1C[C@H](Nc2cnn(C)c(=O)c2Cl)C[C@H](c2ccc(C(=O)N3CCC4(CC3)CCN(c3ccc5c(c3)C(=O)N(C3CCC(=O)NC3=O)C5=O)CC4)cc2)C1] |
| 301a | 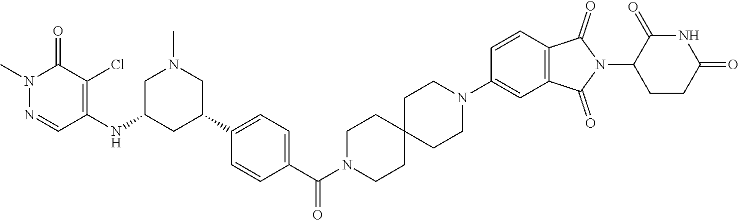 [SMILES: CN1C[C@@H](Nc2cnn(C)c(=O)c2Cl)C[C@@H](c2ccc(C(=O)N3CCC4(CC3)CCN(c3ccc5c(c3)C(=O)N(C3CCC(=O)NC3=O)C5=O)CC4)cc2)C1] |
| 301b | 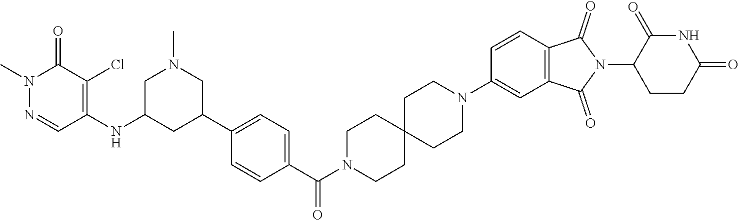 [SMILES: CN1CC(Nc2cnn(C)c(=O)c2Cl)CC(c2ccc(C(=O)N3CCC4(CC3)CCN(c3ccc5c(c3)C(=O)N(C3CCC(=O)NC3=O)C5=O)CC4)cc2)C1] |
| 302 | 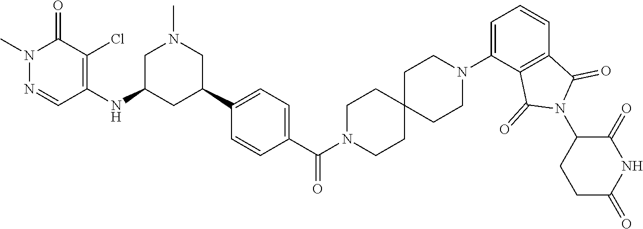 [SMILES: CN1C[C@H](Nc2cnn(C)c(=O)c2Cl)C[C@H](c2ccc(C(=O)N3CCC4(CC3)CCN(c3cccc5c3C(=O)N(C3CCC(=O)NC3=O)C5=O)CC4)cc2)C1] |
| 302a | 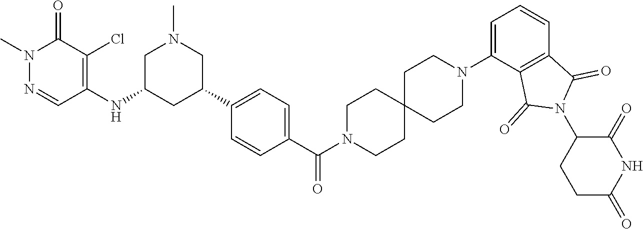 [SMILES: CN1C[C@@H](Nc2cnn(C)c(=O)c2Cl)C[C@@H](c2ccc(C(=O)N3CCC4(CC3)CCN(c3cccc5c3C(=O)N(C3CCC(=O)NC3=O)C5=O)CC4)cc2)C1] |
| 302b | 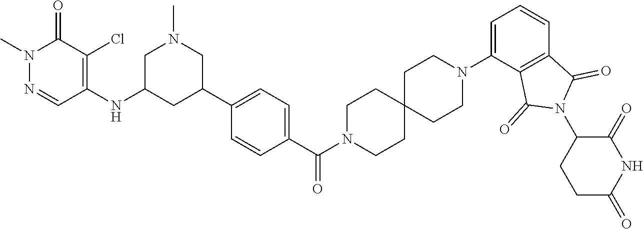 [SMILES: CN1CC(Nc2cnn(C)c(=O)c2Cl)CC(c2ccc(C(=O)N3CCC4(CC3)CCN(c3cccc5c3C(=O)N(C3CCC(=O)NC3=O)C5=O)CC4)cc2)C1] |
| 303 | 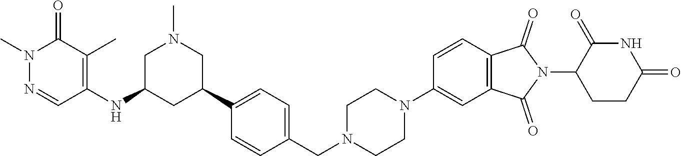 [SMILES: Cc1c(N[C@@H]2C[C@H](c3ccc(CN4CCN(c5ccc6c(c5)C(=O)N(C5CCC(=O)NC5=O)C6=O)CC4)cc3)CN(C)C2)cnn(C)c1=O] |
| 303a | 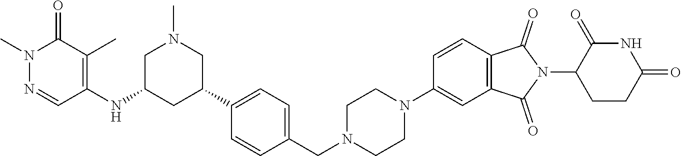 [SMILES: Cc1c(N[C@H]2C[C@@H](c3ccc(CN4CCN(c5ccc6c(c5)C(=O)N(C5CCC(=O)NC5=O)C6=O)CC4)cc3)CN(C)C2)cnn(C)c1=O] |
| 303b | 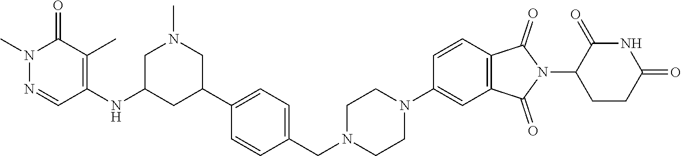 [SMILES: Cc1c(NC2CC(c3ccc(CN4CCN(c5ccc6c(c5)C(=O)N(C5CCC(=O)NC5=O)C6=O)CC4)cc3)CN(C)C2)cnn(C)c1=O] |
| 304 | 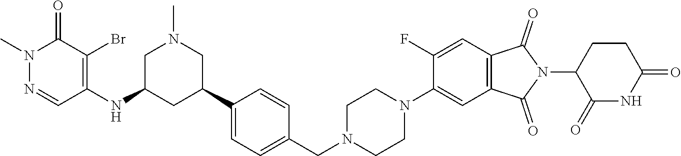 [SMILES: CN1C[C@H](Nc2cnn(C)c(=O)c2Br)C[C@H](c2ccc(CN3CCN(c4cc5c(cc4F)C(=O)N(C4CCC(=O)NC4=O)C5=O)CC3)cc2)C1] |
| 304a | 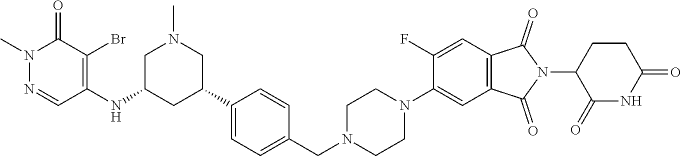 [SMILES: CN1C[C@@H](Nc2cnn(C)c(=O)c2Br)C[C@@H](c2ccc(CN3CCN(c4cc5c(cc4F)C(=O)N(C4CCC(=O)NC4=O)C5=O)CC3)cc2)C1] |
| 304b | 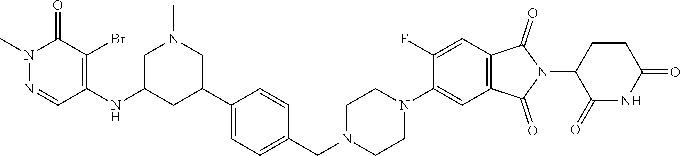 [SMILES: CN1CC(Nc2cnn(C)c(=O)c2Br)CC(c2ccc(CN3CCN(c4cc5c(cc4F)C(=O)N(C4CCC(=O)NC4=O)C5=O)CC3)cc2)C1] |
| 305 | 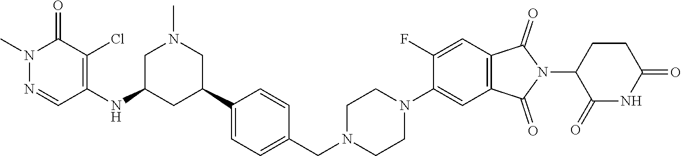 [SMILES: CN1C[C@H](Nc2cnn(C)c(=O)c2Cl)C[C@H](c2ccc(CN3CCN(c4cc5c(cc4F)C(=O)N(C4CCC(=O)NC4=O)C5=O)CC3)cc2)C1] |
| 305a | 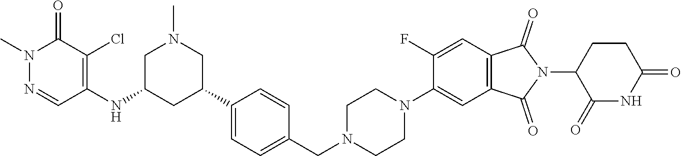 [SMILES: CN1C[C@@H](Nc2cnn(C)c(=O)c2Cl)C[C@@H](c2ccc(CN3CCN(c4cc5c(cc4F)C(=O)N(C4CCC(=O)NC4=O)C5=O)CC3)cc2)C1] |
| 305b | 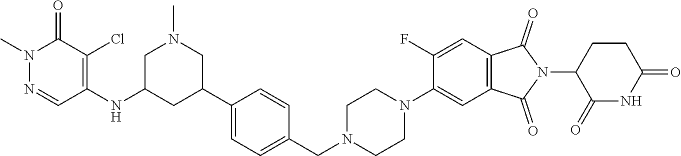 [SMILES: CN1CC(Nc2cnn(C)c(=O)c2Cl)CC(c2ccc(CN3CCN(c4cc5c(cc4F)C(=O)N(C4CCC(=O)NC4=O)C5=O)CC3)cc2)C1] |
| 306 | 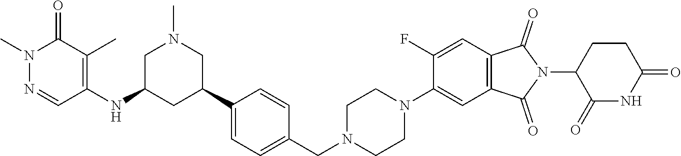 [SMILES: Cc1c(N[C@@H]2C[C@H](c3ccc(CN4CCN(c5cc6c(cc5F)C(=O)N(C5CCC(=O)NC5=O)C6=O)CC4)cc3)CN(C)C2)cnn(C)c1=O] |
| 306a | 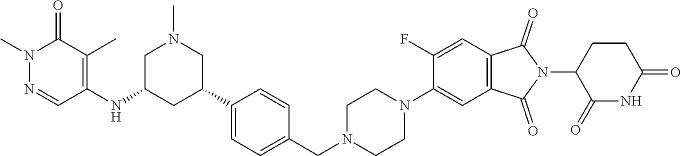 [SMILES: Cc1c(N[C@H]2C[C@@H](c3ccc(CN4CCN(c5cc6c(cc5F)C(=O)N(C5CCC(=O)NC5=O)C6=O)CC4)cc3)CN(C)C2)cnn(C)c1=O] |
| 306b | 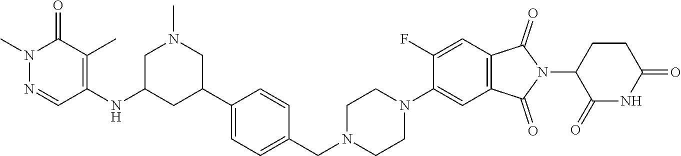 [SMILES: Cc1c(NC2CC(c3ccc(CN4CCN(c5cc6c(cc5F)C(=O)N(C5CCC(=O)NC5=O)C6=O)CC4)cc3)CN(C)C2)cnn(C)c1=O] |
| 307 | 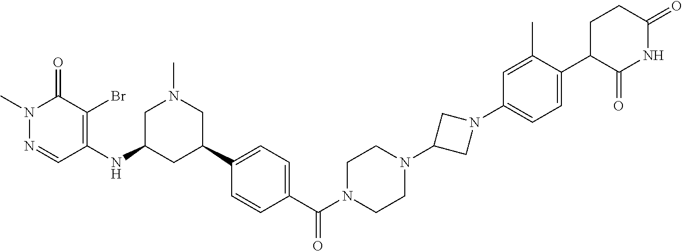 [SMILES: Cc1cc(N2CC(N3CCN(C(=O)c4ccc([C@H]5C[C@@H](Nc6cnn(C)c(=O)c6Br)CN(C)C5)cc4)CC3)C2)ccc1C1CCC(=O)NC1=O] |
| 307a | 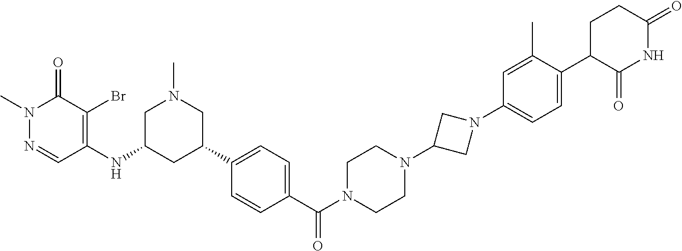 [SMILES: Cc1cc(N2CC(N3CCN(C(=O)c4ccc([C@@H]5C[C@H](Nc6cnn(C)c(=O)c6Br)CN(C)C5)cc4)CC3)C2)ccc1C1CCC(=O)NC1=O] |
| 307b | 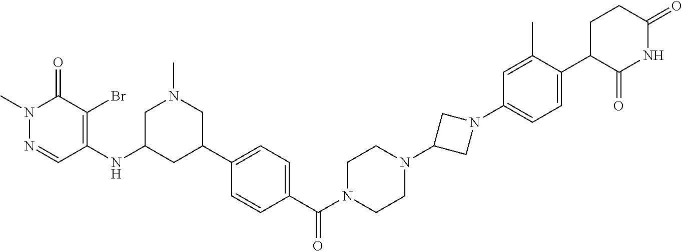 [SMILES: Cc1cc(N2CC(N3CCN(C(=O)c4ccc(C5CC(Nc6cnn(C)c(=O)c6Br)CN(C)C5)cc4)CC3)C2)ccc1C1CCC(=O)NC1=O] |
| 308 | 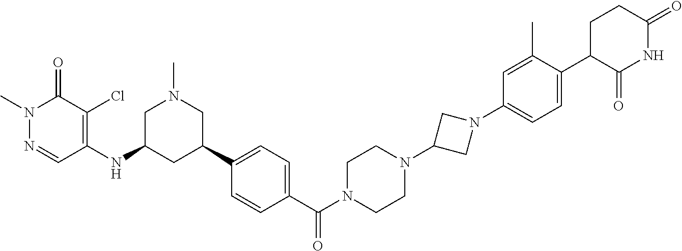 [SMILES: Cc1cc(N2CC(N3CCN(C(=O)c4ccc([C@H]5C[C@@H](Nc6cnn(C)c(=O)c6Cl)CN(C)C5)cc4)CC3)C2)ccc1C1CCC(=O)NC1=O] |
| 308a | 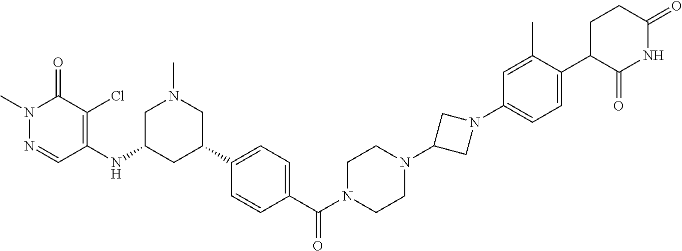 [SMILES: Cc1cc(N2CC(N3CCN(C(=O)c4ccc([C@@H]5C[C@H](Nc6cnn(C)c(=O)c6Cl)CN(C)C5)cc4)CC3)C2)ccc1C1CCC(=O)NC1=O] |
| 308b | 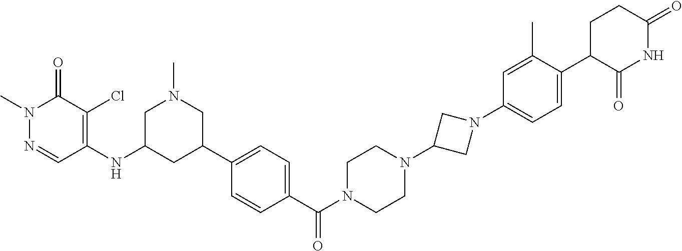 [SMILES: Cc1cc(N2CC(N3CCN(C(=O)c4ccc(C5CC(Nc6cnn(C)c(=O)c6Cl)CN(C)C5)cc4)CC3)C2)ccc1C1CCC(=O)NC1=O] |
| 309 | 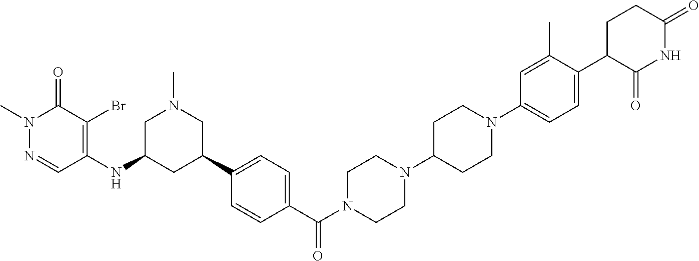 [SMILES: Cc1cc(N2CCC(N3CCN(C(=O)c4ccc([C@H]5C[C@@H](Nc6cnn(C)c(=O)c6Br)CN(C)C5)cc4)CC3)CC2)ccc1C1CCC(=O)NC1=O] |
| 309a | 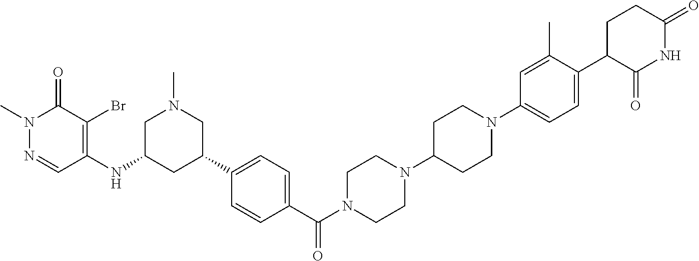 [SMILES: Cc1cc(N2CCC(N3CCN(C(=O)c4ccc([C@@H]5C[C@H](Nc6cnn(C)c(=O)c6Br)CN(C)C5)cc4)CC3)CC2)ccc1C1CCC(=O)NC1=O] |
| 309b | 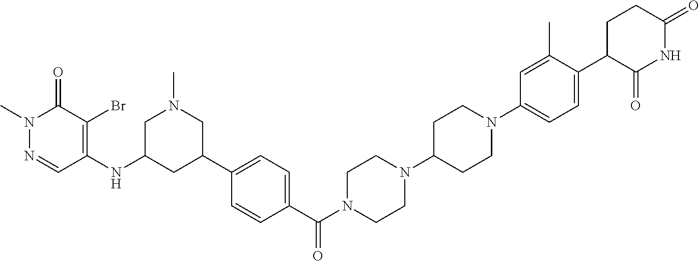 [SMILES: Cc1cc(N2CCC(N3CCN(C(=O)c4ccc(C5CC(Nc6cnn(C)c(=O)c6Br)CN(C)C5)cc4)CC3)CC2)ccc1C1CCC(=O)NC1=O] |
| 310 | 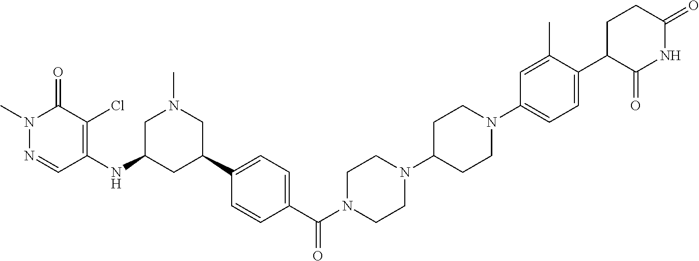 [SMILES: Cc1cc(N2CCC(N3CCN(C(=O)c4ccc([C@H]5C[C@@H](Nc6cnn(C)c(=O)c6Cl)CN(C)C5)cc4)CC3)CC2)ccc1C1CCC(=O)NC1=O] |
| 310a | 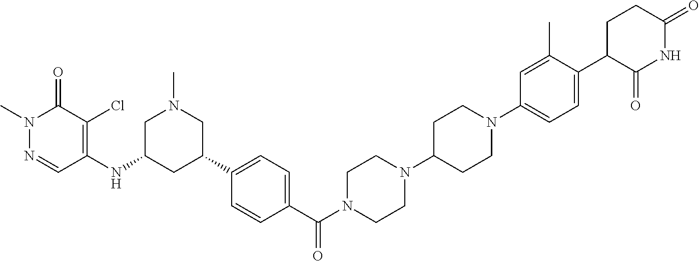 [SMILES: Cc1cc(N2CCC(N3CCN(C(=O)c4ccc([C@@H]5C[C@H](Nc6cnn(C)c(=O)c6Cl)CN(C)C5)cc4)CC3)CC2)ccc1C1CCC(=O)NC1=O] |
| 310b | 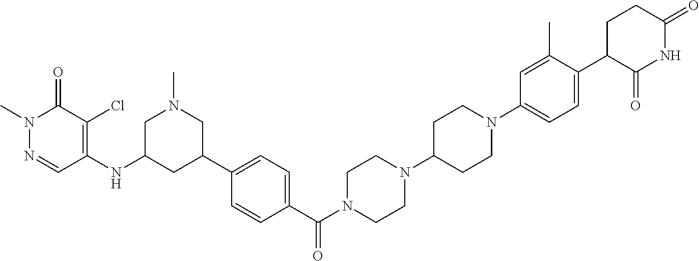 [SMILES: Cc1cc(N2CCC(N3CCN(C(=O)c4ccc(C5CC(Nc6cnn(C)c(=O)c6Cl)CN(C)C5)cc4)CC3)CC2)ccc1C1CCC(=O)NC1=O] |
| 311 | 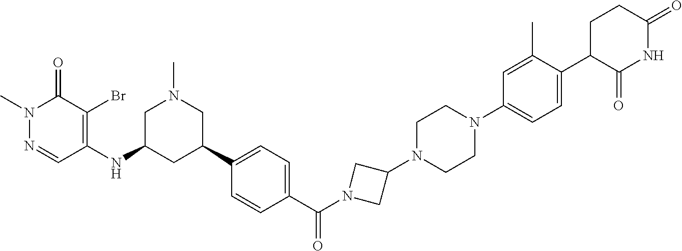 [SMILES: Cc1cc(N2CCN(C3CN(C(=O)c4ccc([C@H]5C[C@@H](Nc6cnn(C)c(=O)c6Br)CN(C)C5)cc4)C3)CC2)ccc1C1CCC(=O)NC1=O] |
| 311a | 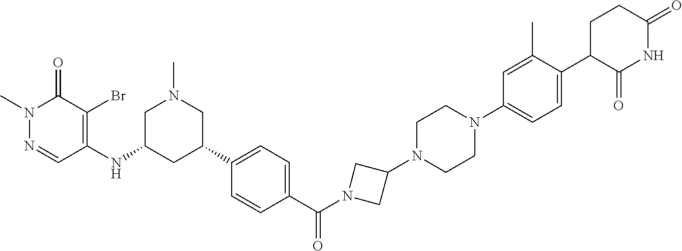 [SMILES: Cc1cc(N2CCN(C3CN(C(=O)c4ccc([C@@H]5C[C@H](Nc6cnn(C)c(=O)c6Br)CN(C)C5)cc4)C3)CC2)ccc1C1CCC(=O)NC1=O] |
| 311b | 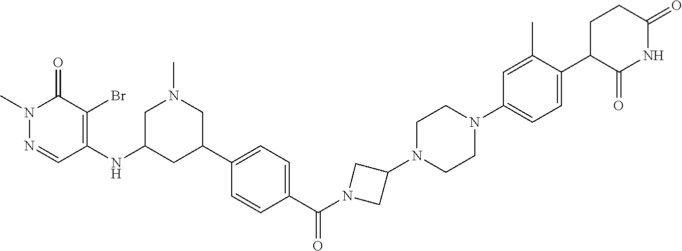 [SMILES: Cc1cc(N2CCN(C3CN(C(=O)c4ccc(C5CC(Nc6cnn(C)c(=O)c6Br)CN(C)C5)cc4)C3)CC2)ccc1C1CCC(=O)NC1=O] |
| 312 | 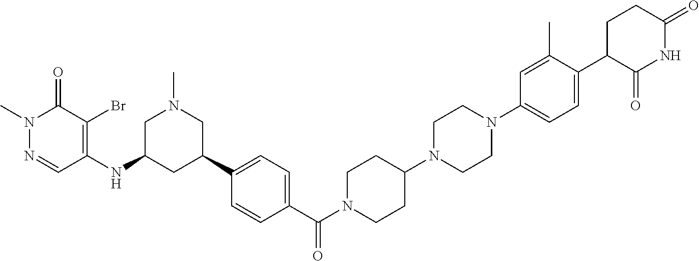 [SMILES: Cc1cc(N2CCN(C3CCN(C(=O)c4ccc([C@H]5C[C@@H](Nc6cnn(C)c(=O)c6Br)CN(C)C5)cc4)CC3)CC2)ccc1C1CCC(=O)NC1=O] |
| 312a | 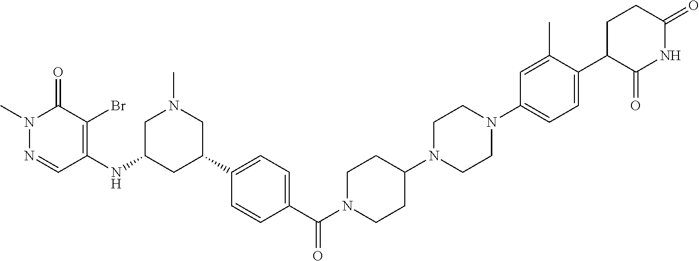 [SMILES: Cc1cc(N2CCN(C3CCN(C(=O)c4ccc([C@@H]5C[C@H](Nc6cnn(C)c(=O)c6Br)CN(C)C5)cc4)CC3)CC2)ccc1C1CCC(=O)NC1=O] |
| 312b | 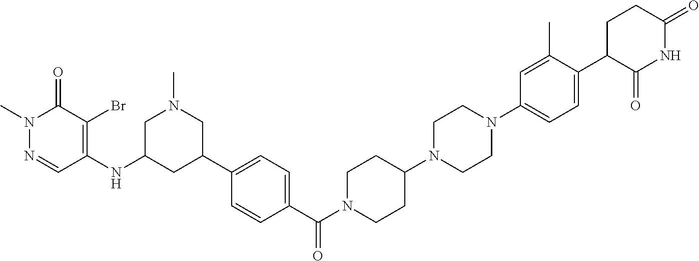 [SMILES: Cc1cc(N2CCN(C3CCN(C(=O)c4ccc(C5CC(Nc6cnn(C)c(=O)c6Br)CN(C)C5)cc4)CC3)CC2)ccc1C1CCC(=O)NC1=O] |
| 313 | 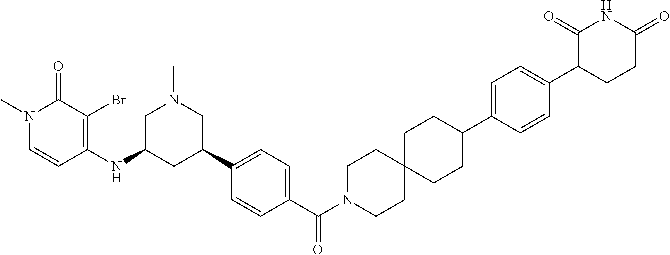 [SMILES: CN1C[C@H](Nc2ccn(C)c(=O)c2Br)C[C@H](c2ccc(C(=O)N3CCC4(CCC(c5ccc(C6CCC(=O)NC6=O)cc5)CC4)CC3)cc2)C1] |
| 313a | 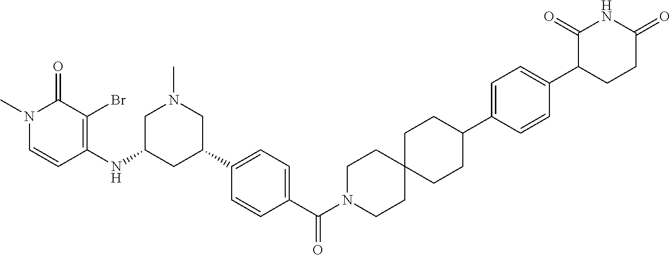 [SMILES: CN1C[C@@H](Nc2ccn(C)c(=O)c2Br)C[C@@H](c2ccc(C(=O)N3CCC4(CCC(c5ccc(C6CCC(=O)NC6=O)cc5)CC4)CC3)cc2)C1] |
| 313b | 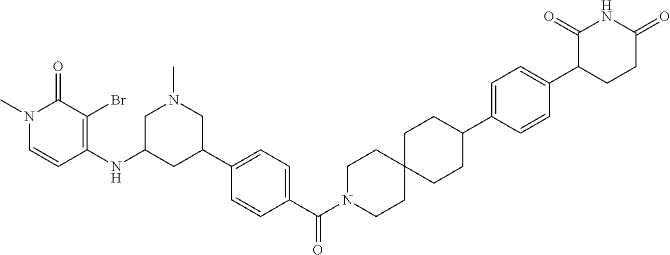 [SMILES: CN1CC(Nc2ccn(C)c(=O)c2Br)CC(c2ccc(C(=O)N3CCC4(CCC(c5ccc(C6CCC(=O)NC6=O)cc5)CC4)CC3)cc2)C1] |
| 314 | 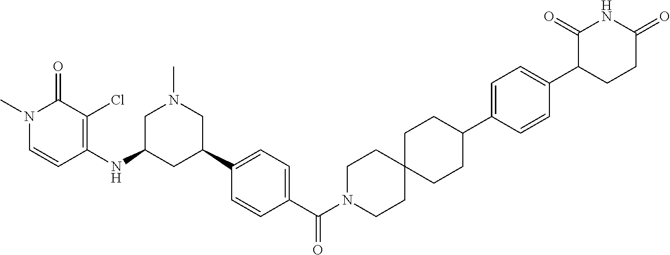 [SMILES: CN1C[C@H](Nc2ccn(C)c(=O)c2Cl)C[C@H](c2ccc(C(=O)N3CCC4(CCC(c5ccc(C6CCC(=O)NC6=O)cc5)CC4)CC3)cc2)C1] |
| 314a | 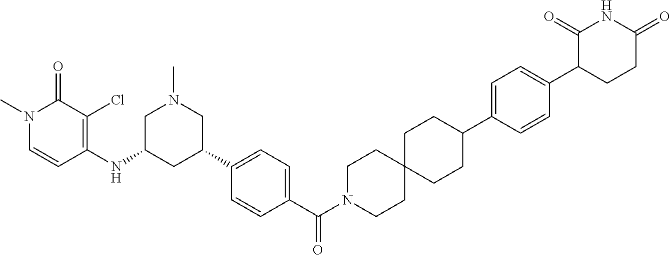 [SMILES: CN1C[C@@H](Nc2ccn(C)c(=O)c2Cl)C[C@@H](c2ccc(C(=O)N3CCC4(CCC(c5ccc(C6CCC(=O)NC6=O)cc5)CC4)CC3)cc2)C1] |
| 314b | 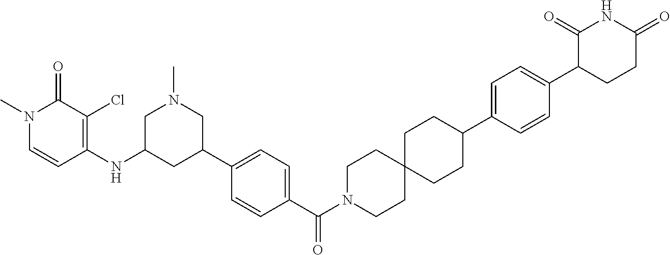 [SMILES: CN1CC(Nc2ccn(C)c(=O)c2Cl)CC(c2ccc(C(=O)N3CCC4(CCC(c5ccc(C6CCC(=O)NC6=O)cc5)CC4)CC3)cc2)C1] |
| 315 | 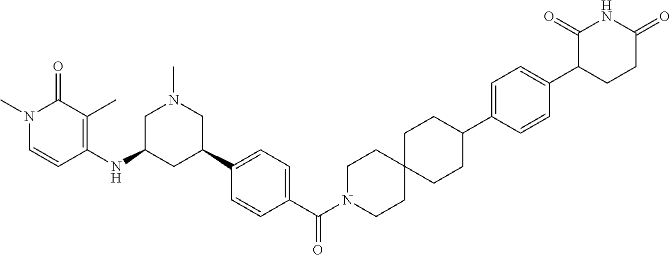 [SMILES: Cc1c(N[C@@H]2C[C@H](c3ccc(C(=O)N4CCC5(CCC(c6ccc(C7CCC(=O)NC7=O)cc6)CC5)CC4)cc3)CN(C)C2)ccn(C)c1=O] |
| 315a | 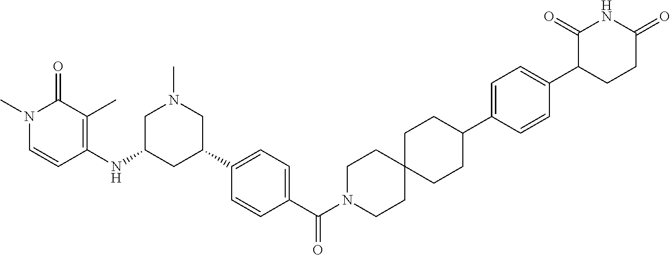 [SMILES: Cc1c(N[C@H]2C[C@@H](c3ccc(C(=O)N4CCC5(CCC(c6ccc(C7CCC(=O)NC7=O)cc6)CC5)CC4)cc3)CN(C)C2)ccn(C)c1=O] |
| 315b | 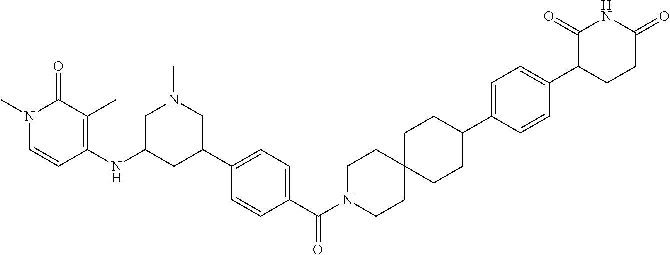 [SMILES: Cc1c(NC2CC(c3ccc(C(=O)N4CCC5(CCC(c6ccc(C7CCC(=O)NC7=O)cc6)CC5)CC4)cc3)CN(C)C2)ccn(C)c1=O] |
| 316 | 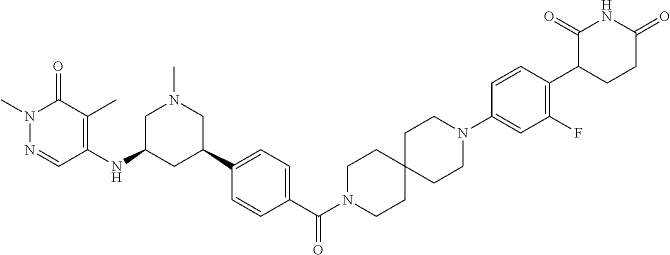 [SMILES: Cc1c(N[C@@H]2C[C@H](c3ccc(C(=O)N4CCC5(CC4)CCN(c4ccc(C6CCC(=O)NC6=O)c(F)c4)CC5)cc3)CN(C)C2)cnn(C)c1=O] |
| 316a | 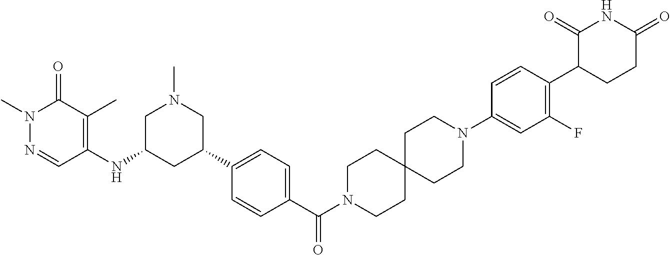 [SMILES: Cc1c(N[C@H]2C[C@@H](c3ccc(C(=O)N4CCC5(CC4)CCN(c4ccc(C6CCC(=O)NC6=O)c(F)c4)CC5)cc3)CN(C)C2)cnn(C)c1=O] |
| 316b | 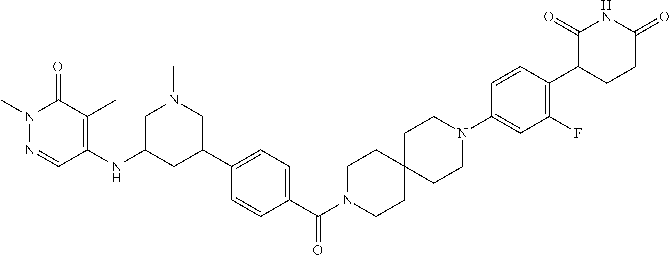 [SMILES: Cc1c(NC2CC(c3ccc(C(=O)N4CCC5(CC4)CCN(c4ccc(C6CCC(=O)NC6=O)c(F)c4)CC5)cc3)CN(C)C2)cnn(C)c1=O] |
| 317 | 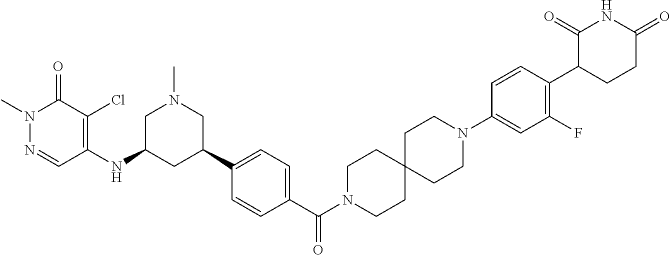 [SMILES: CN1C[C@H](Nc2cnn(C)c(=O)c2Cl)C[C@H](c2ccc(C(=O)N3CCC4(CC3)CCN(c3ccc(C5CCC(=O)NC5=O)c(F)c3)CC4)cc2)C1] |
| 317a | 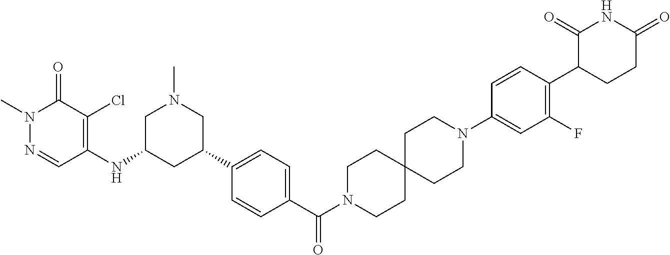 [SMILES: CN1C[C@@H](Nc2cnn(C)c(=O)c2Cl)C[C@@H](c2ccc(C(=O)N3CCC4(CC3)CCN(c3ccc(C5CCC(=O)NC5=O)c(F)c3)CC4)cc2)C1] |
| 317b | 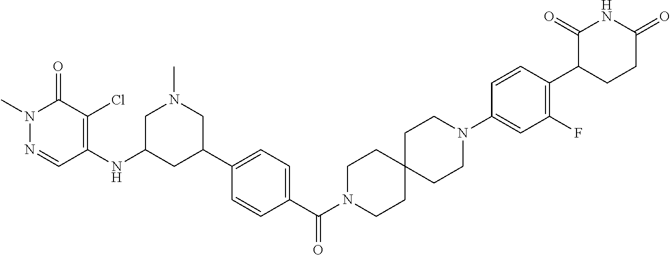 [SMILES: CN1CC(Nc2cnn(C)c(=O)c2Cl)CC(c2ccc(C(=O)N3CCC4(CC3)CCN(c3ccc(C5CCC(=O)NC5=O)c(F)c3)CC4)cc2)C1] |
| 318 | 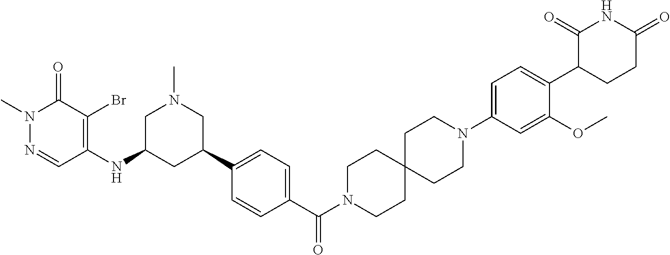 [SMILES: COc1cc(N2CCC3(CCN(C(=O)c4ccc([C@H]5C[C@@H](Nc6cnn(C)c(=O)c6Br)CN(C)C5)cc4)CC3)CC2)ccc1C1CCC(=O)NC1=O] |
| 318a | 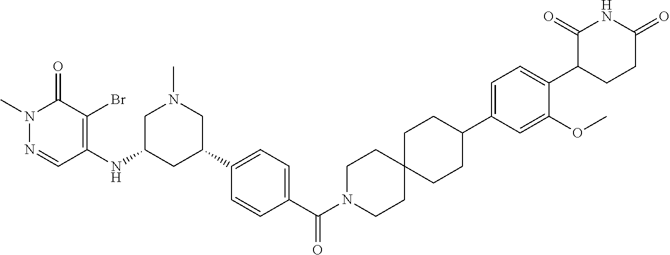 [SMILES: COc1cc(C2CCC3(CC2)CCN(C(=O)c2ccc([C@@H]4C[C@H](Nc5cnn(C)c(=O)c5Br)CN(C)C4)cc2)CC3)ccc1C1CCC(=O)NC1=O] |
| 318b | 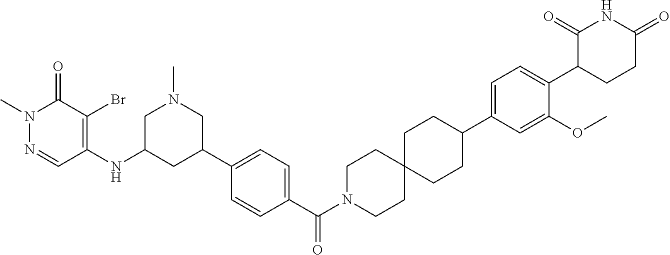 [SMILES: COc1cc(C2CCC3(CC2)CCN(C(=O)c2ccc(C4CC(Nc5cnn(C)c(=O)c5Br)CN(C)C4)cc2)CC3)ccc1C1CCC(=O)NC1=O] |
| 319 | 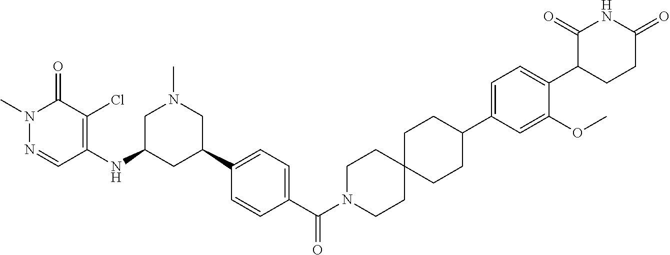 [SMILES: COc1cc(C2CCC3(CC2)CCN(C(=O)c2ccc([C@H]4C[C@@H](Nc5cnn(C)c(=O)c5Cl)CN(C)C4)cc2)CC3)ccc1C1CCC(=O)NC1=O] |
| 319a | 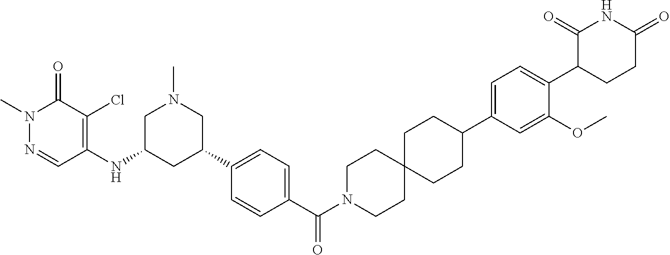 [SMILES: COc1cc(C2CCC3(CC2)CCN(C(=O)c2ccc([C@@H]4C[C@H](Nc5cnn(C)c(=O)c5Cl)CN(C)C4)cc2)CC3)ccc1C1CCC(=O)NC1=O] |
| 319b | 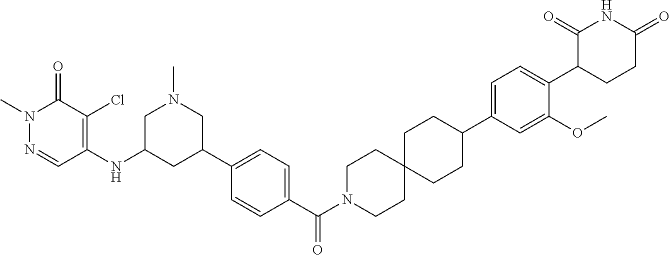 [SMILES: COc1cc(C2CCC3(CC2)CCN(C(=O)c2ccc(C4CC(Nc5cnn(C)c(=O)c5Cl)CN(C)C4)cc2)CC3)ccc1C1CCC(=O)NC1=O] |
| 320 | 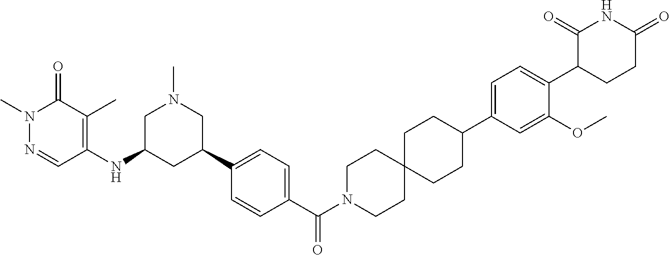 [SMILES: COc1cc(C2CCC3(CC2)CCN(C(=O)c2ccc([C@H]4C[C@@H](Nc5cnn(C)c(=O)c5C)CN(C)C4)cc2)CC3)ccc1C1CCC(=O)NC1=O] |
| 320a | 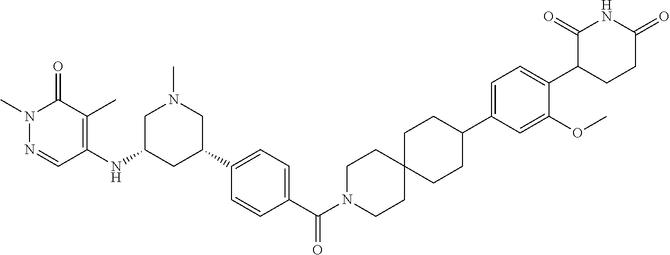 [SMILES: COc1cc(C2CCC3(CC2)CCN(C(=O)c2ccc([C@@H]4C[C@H](Nc5cnn(C)c(=O)c5C)CN(C)C4)cc2)CC3)ccc1C1CCC(=O)NC1=O] |
| 320b | 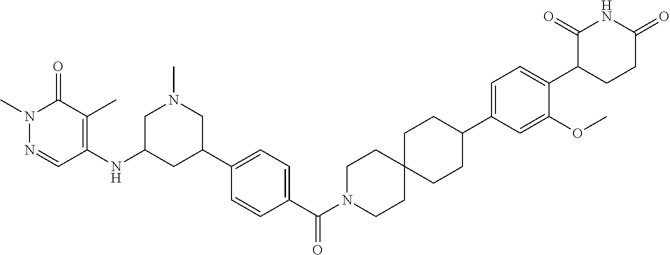 [SMILES: COc1cc(C2CCC3(CC2)CCN(C(=O)c2ccc(C4CC(Nc5cnn(C)c(=O)c5C)CN(C)C4)cc2)CC3)ccc1C1CCC(=O)NC1=O] |
| 321 | 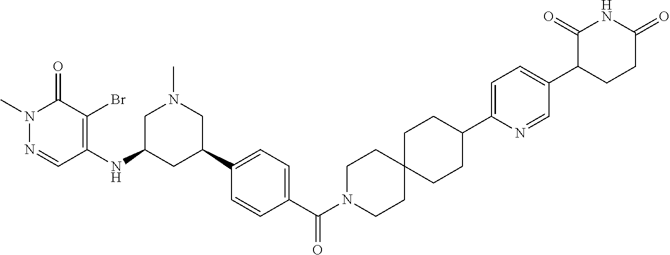 [SMILES: CN1C[C@H](Nc2cnn(C)c(=O)c2Br)C[C@H](c2ccc(C(=O)N3CCC4(CCC(c5ccc(C6CCC(=O)NC6=O)cn5)CC4)CC3)cc2)C1] |
| 321a | 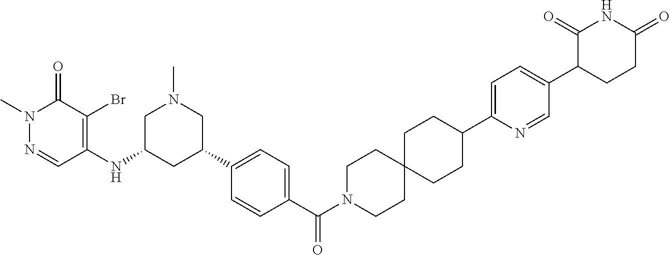 [SMILES: CN1C[C@@H](Nc2cnn(C)c(=O)c2Br)C[C@@H](c2ccc(C(=O)N3CCC4(CCC(c5ccc(C6CCC(=O)NC6=O)cn5)CC4)CC3)cc2)C1] |
| 321b | 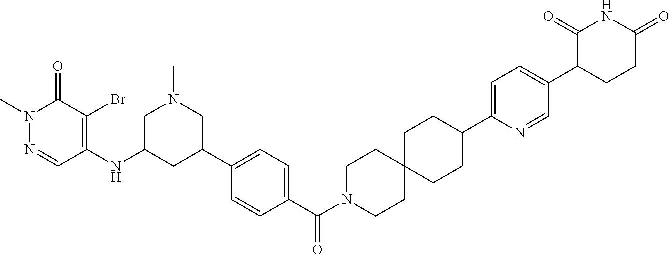 [SMILES: CN1CC(Nc2cnn(C)c(=O)c2Br)CC(c2ccc(C(=O)N3CCC4(CCC(c5ccc(C6CCC(=O)NC6=O)cn5)CC4)CC3)cc2)C1] |
| 322 | 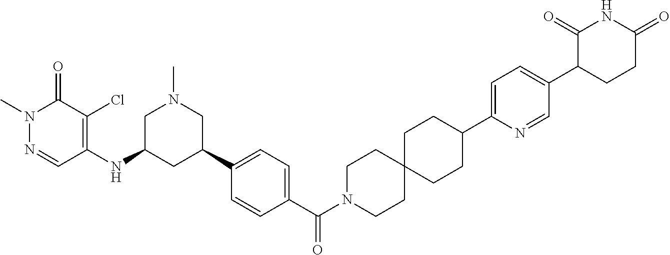 [SMILES: CN1C[C@H](Nc2cnn(C)c(=O)c2Cl)C[C@H](c2ccc(C(=O)N3CCC4(CCC(c5ccc(C6CCC(=O)NC6=O)cn5)CC4)CC3)cc2)C1] |
| 322a | 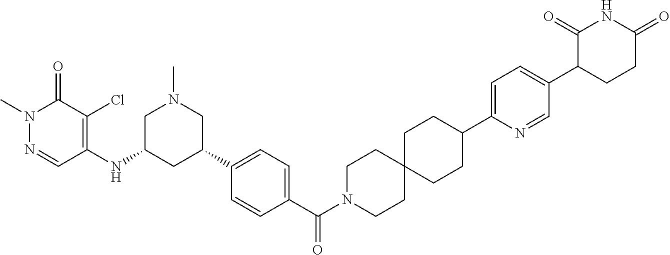 [SMILES: CN1C[C@@H](Nc2cnn(C)c(=O)c2Cl)C[C@@H](c2ccc(C(=O)N3CCC4(CCC(c5ccc(C6CCC(=O)NC6=O)cn5)CC4)CC3)cc2)C1] |
| 322b | 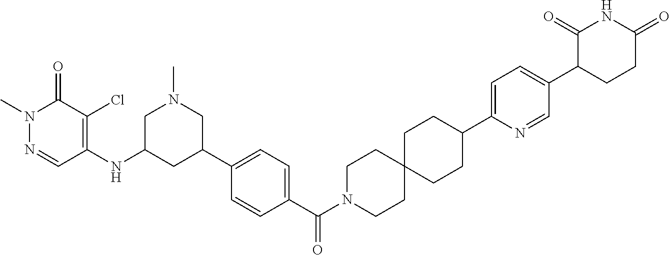 [SMILES: CN1CC(Nc2cnn(C)c(=O)c2Cl)CC(c2ccc(C(=O)N3CCC4(CCC(c5ccc(C6CCC(=O)NC6=O)cn5)CC4)CC3)cc2)C1] |
| 323 | 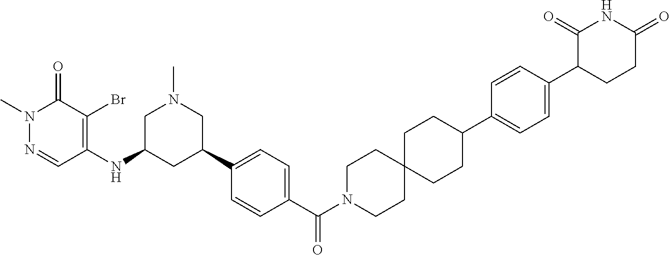 [SMILES: CN1C[C@H](Nc2cnn(C)c(=O)c2Br)C[C@H](c2ccc(C(=O)N3CCC4(CCC(c5ccc(C6CCC(=O)NC6=O)cc5)CC4)CC3)cc2)C1] |
| 323a | 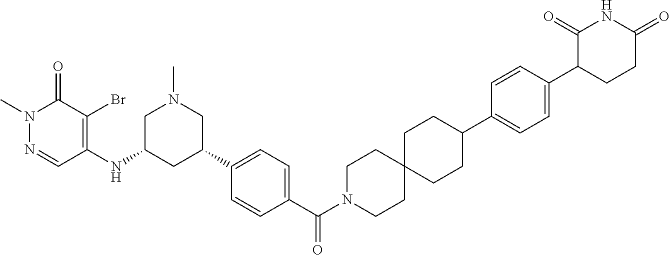 [SMILES: CN1C[C@@H](Nc2cnn(C)c(=O)c2Br)C[C@@H](c2ccc(C(=O)N3CCC4(CCC(c5ccc(C6CCC(=O)NC6=O)cc5)CC4)CC3)cc2)C1] |
| 323b | 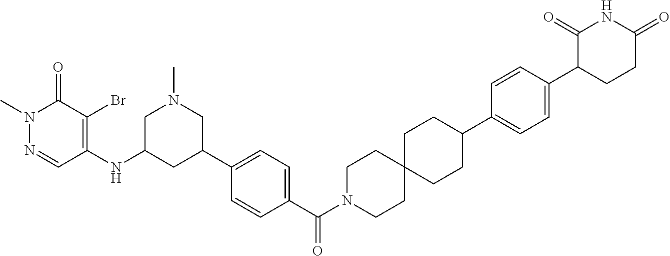 [SMILES: CN1CC(Nc2cnn(C)c(=O)c2Br)CC(c2ccc(C(=O)N3CCC4(CCC(c5ccc(C6CCC(=O)NC6=O)cc5)CC4)CC3)cc2)C1] |
| 324 | 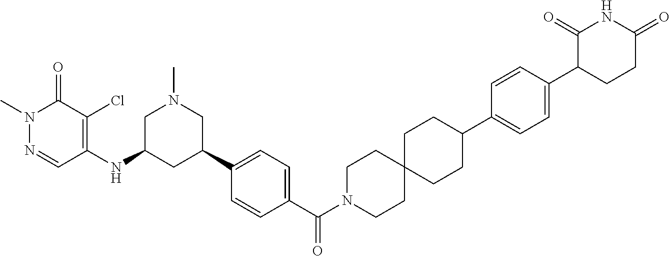 [SMILES: CN1C[C@H](Nc2cnn(C)c(=O)c2Cl)C[C@H](c2ccc(C(=O)N3CCC4(CCC(c5ccc(C6CCC(=O)NC6=O)cc5)CC4)CC3)cc2)C1] |
| 324a | 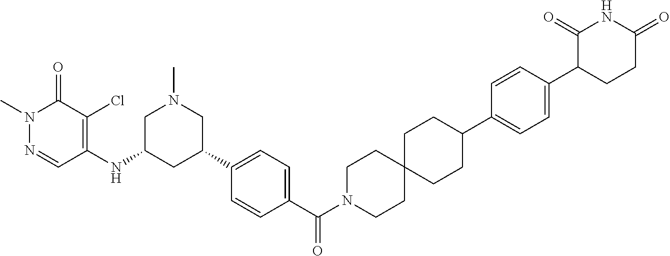 [SMILES: CN1C[C@@H](Nc2cnn(C)c(=O)c2Cl)C[C@@H](c2ccc(C(=O)N3CCC4(CCC(c5ccc(C6CCC(=O)NC6=O)cc5)CC4)CC3)cc2)C1] |
| 324b | 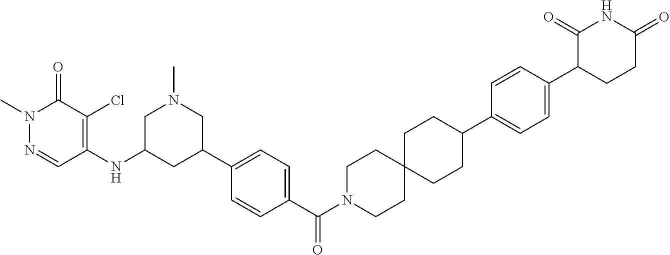 [SMILES: CN1CC(Nc2cnn(C)c(=O)c2Cl)CC(c2ccc(C(=O)N3CCC4(CCC(c5ccc(C6CCC(=O)NC6=O)cc5)CC4)CC3)cc2)C1] |
| 325 | 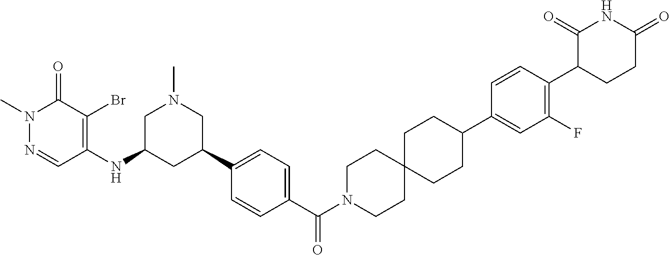 [SMILES: CN1C[C@H](Nc2cnn(C)c(=O)c2Br)C[C@H](c2ccc(C(=O)N3CCC4(CCC(c5ccc(C6CCC(=O)NC6=O)c(F)c5)CC4)CC3)cc2)C1] |
| 325a | 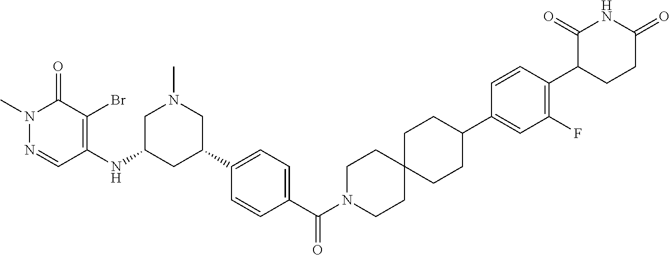 [SMILES: CN1C[C@@H](Nc2cnn(C)c(=O)c2Br)C[C@@H](c2ccc(C(=O)N3CCC4(CCC(c5ccc(C6CCC(=O)NC6=O)c(F)c5)CC4)CC3)cc2)C1] |
| 325b | 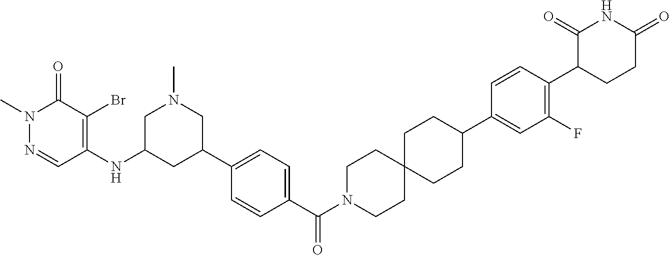 [SMILES: CN1CC(Nc2cnn(C)c(=O)c2Br)CC(c2ccc(C(=O)N3CCC4(CCC(c5ccc(C6CCC(=O)NC6=O)c(F)c5)CC4)CC3)cc2)C1] |
| 326 | 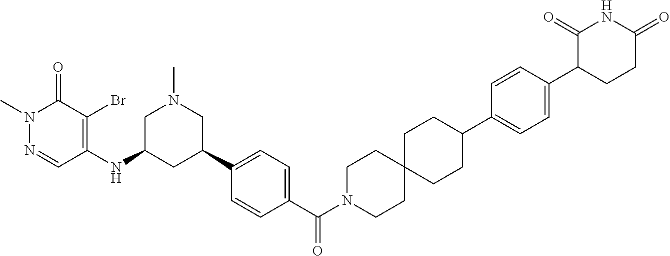 [SMILES: CN1C[C@H](Nc2cnn(C)c(=O)c2Br)C[C@H](c2ccc(C(=O)N3CCC4(CCC(c5ccc(C6CCC(=O)NC6=O)cc5)CC4)CC3)cc2)C1] |
| 326a | 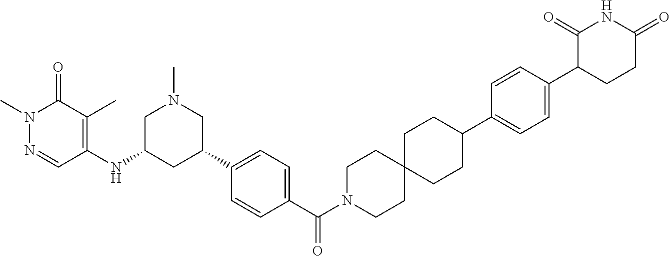 [SMILES: Cc1c(N[C@H]2C[C@@H](c3ccc(C(=O)N4CCC5(CCC(c6ccc(C7CCC(=O)NC7=O)cc6)CC5)CC4)cc3)CN(C)C2)cnn(C)c1=O] |
| 326b | 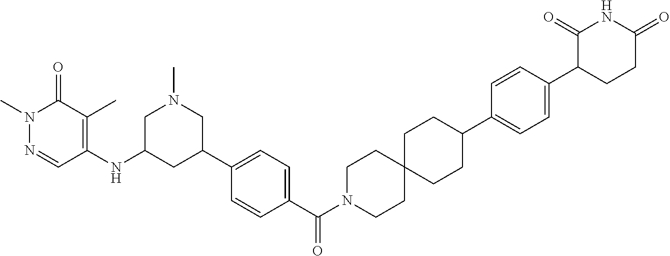 [SMILES: Cc1c(NC2CC(c3ccc(C(=O)N4CCC5(CCC(c6ccc(C7CCC(=O)NC7=O)cc6)CC5)CC4)cc3)CN(C)C2)cnn(C)c1=O] |
| 327 | 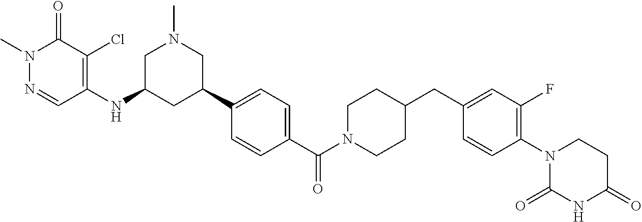 [SMILES: CN1C[C@H](Nc2cnn(C)c(=O)c2Cl)C[C@H](c2ccc(C(=O)N3CCC(Cc4ccc(N5CCC(=O)NC5=O)c(F)c4)CC3)cc2)C1] |
| 327a | 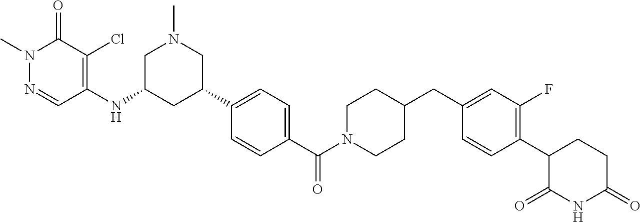 [SMILES: CN1C[C@@H](Nc2cnn(C)c(=O)c2Cl)C[C@@H](c2ccc(C(=O)N3CCC(Cc4ccc(C5CCC(=O)NC5=O)c(F)c4)CC3)cc2)C1] |
| 327b | 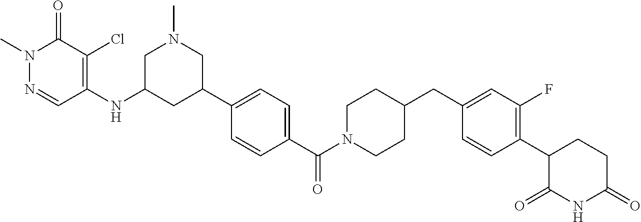 [SMILES: CN1CC(Nc2cnn(C)c(=O)c2Cl)CC(c2ccc(C(=O)N3CCC(Cc4ccc(C5CCC(=O)NC5=O)c(F)c4)CC3)cc2)C1] |
| 328 | 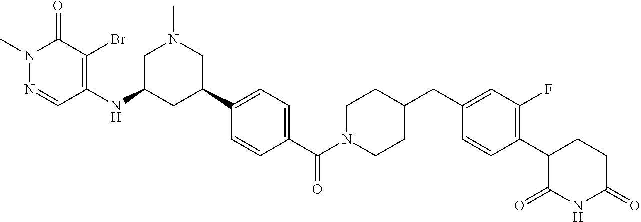 [SMILES: CN1C[C@H](Nc2cnn(C)c(=O)c2Br)C[C@H](c2ccc(C(=O)N3CCC(Cc4ccc(C5CCC(=O)NC5=O)c(F)c4)CC3)cc2)C1] |
| 328a | 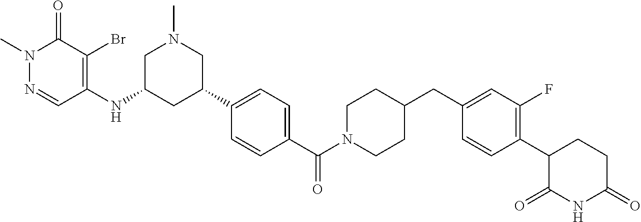 [SMILES: CN1C[C@@H](Nc2cnn(C)c(=O)c2Br)C[C@@H](c2ccc(C(=O)N3CCC(Cc4ccc(C5CCC(=O)NC5=O)c(F)c4)CC3)cc2)C1] |
| 328b | 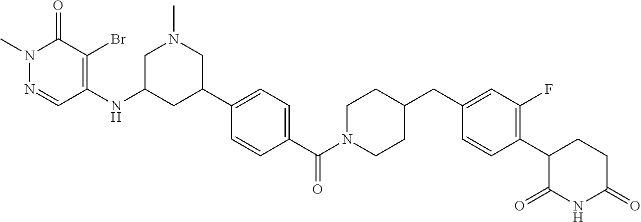 [SMILES: CN1CC(Nc2cnn(C)c(=O)c2Br)CC(c2ccc(C(=O)N3CCC(Cc4ccc(C5CCC(=O)NC5=O)c(F)c4)CC3)cc2)C1] |
| 329 | 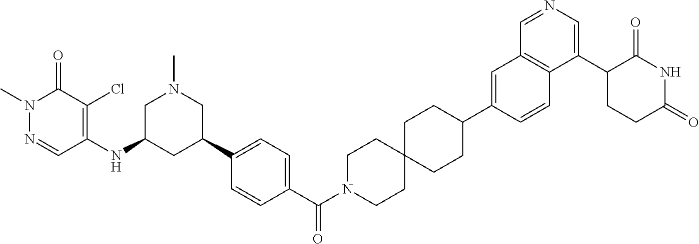 [SMILES: CN1C[C@H](Nc2cnn(C)c(=O)c2Cl)C[C@H](c2ccc(C(=O)N3CCC4(CCC(c5ccc6c(C7CCC(=O)NC7=O)cncc6c5)CC4)CC3)cc2)C1] |
| 329a | 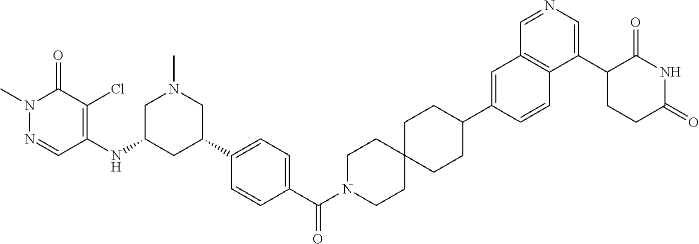 [SMILES: CN1C[C@@H](Nc2cnn(C)c(=O)c2Cl)C[C@@H](c2ccc(C(=O)N3CCC4(CCC(c5ccc6c(C7CCC(=O)NC7=O)cncc6c5)CC4)CC3)cc2)C1] |
| 329b | 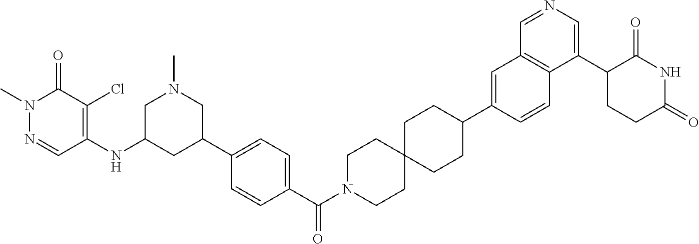 [SMILES: CN1CC(Nc2cnn(C)c(=O)c2Cl)CC(c2ccc(C(=O)N3CCC4(CCC(c5ccc6c(C7CCC(=O)NC7=O)cncc6c5)CC4)CC3)cc2)C1] |
| 330 | 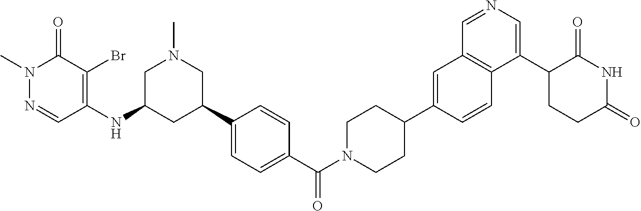 [SMILES: CN1C[C@H](Nc2cnn(C)c(=O)c2Br)C[C@H](c2ccc(C(=O)N3CCC(c4ccc5c(C6CCC(=O)NC6=O)cncc5c4)CC3)cc2)C1] |
| 330a | 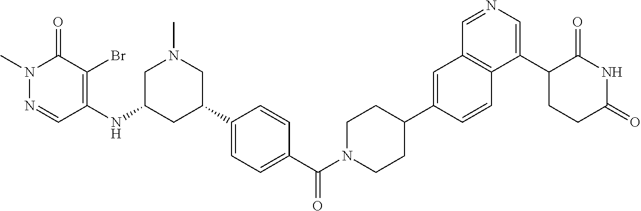 [SMILES: CN1C[C@@H](Nc2cnn(C)c(=O)c2Br)C[C@@H](c2ccc(C(=O)N3CCC(c4ccc5c(C6CCC(=O)NC6=O)cncc5c4)CC3)cc2)C1] |
| 330b | 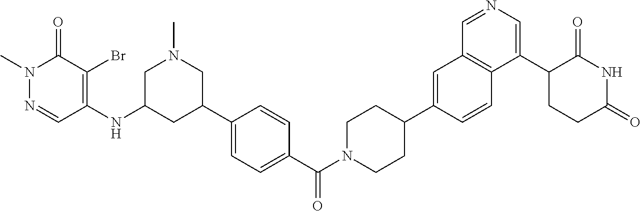 [SMILES: CN1CC(Nc2cnn(C)c(=O)c2Br)CC(c2ccc(C(=O)N3CCC(c4ccc5c(C6CCC(=O)NC6=O)cncc5c4)CC3)cc2)C1] |
| 331 | 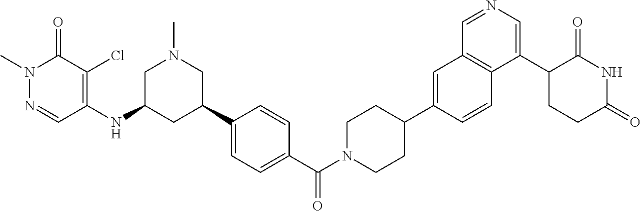 [SMILES: CN1C[C@H](Nc2cnn(C)c(=O)c2Cl)C[C@H](c2ccc(C(=O)N3CCC(c4ccc5c(C6CCC(=O)NC6=O)cncc5c4)CC3)cc2)C1] |
| 331a | 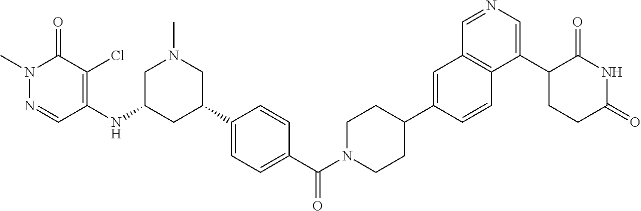 [SMILES: CN1C[C@@H](Nc2cnn(C)c(=O)c2Cl)C[C@@H](c2ccc(C(=O)N3CCC(c4ccc5c(C6CCC(=O)NC6=O)cncc5c4)CC3)cc2)C1] |
| 331b | 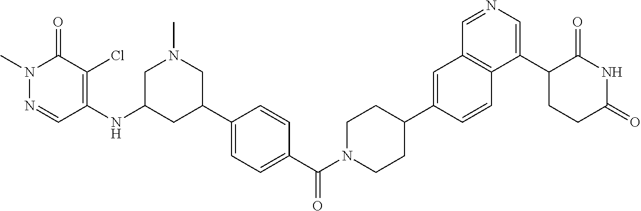 [SMILES: CN1CC(Nc2cnn(C)c(=O)c2Cl)CC(c2ccc(C(=O)N3CCC(c4ccc5c(C6CCC(=O)NC6=O)cncc5c4)CC3)cc2)C1] |
| 332 | 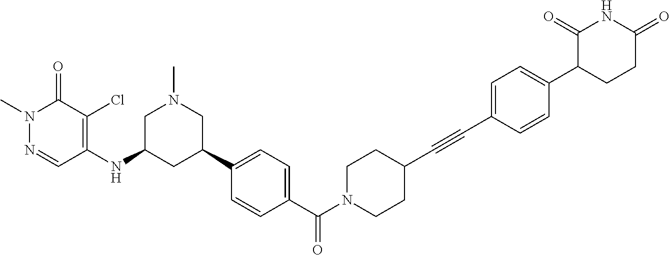 [SMILES: CN1C[C@H](Nc2cnn(C)c(=O)c2Cl)C[C@H](c2ccc(C(=O)N3CCC(C#Cc4ccc(C5CCC(=O)NC5=O)cc4)CC3)cc2)C1] |
| 332a | 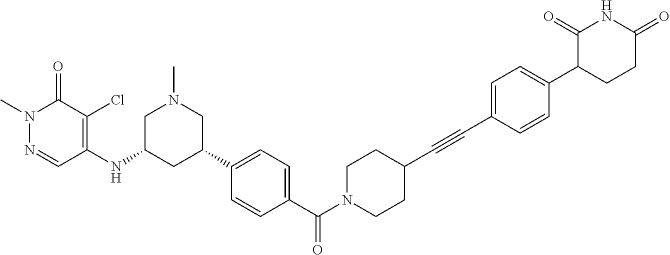 [SMILES: CN1C[C@@H](Nc2cnn(C)c(=O)c2Cl)C[C@@H](c2ccc(C(=O)N3CCC(C#Cc4ccc(C5CCC(=O)NC5=O)cc4)CC3)cc2)C1] |
| 332b | 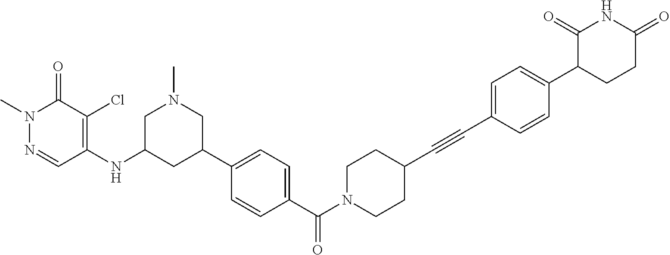 [SMILES: CN1CC(Nc2cnn(C)c(=O)c2Cl)CC(c2ccc(C(=O)N3CCC(C#Cc4ccc(C5CCC(=O)NC5=O)cc4)CC3)cc2)C1] |
| 333 | 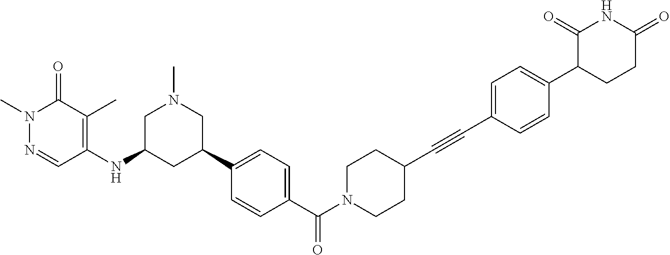 [SMILES: Cc1c(N[C@@H]2C[C@H](c3ccc(C(=O)N4CCC(C#Cc5ccc(C6CCC(=O)NC6=O)cc5)CC4)cc3)CN(C)C2)cnn(C)c1=O] |
| 333a | 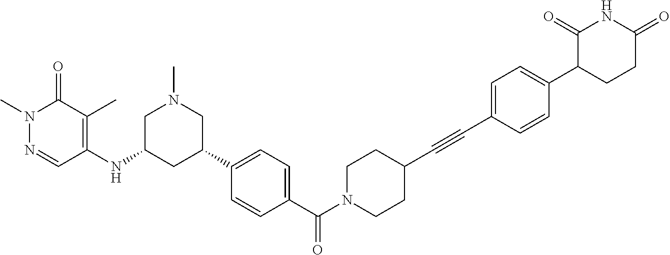 [SMILES: Cc1c(N[C@H]2C[C@@H](c3ccc(C(=O)N4CCC(C#Cc5ccc(C6CCC(=O)NC6=O)cc5)CC4)cc3)CN(C)C2)cnn(C)c1=O] |
| 333b | 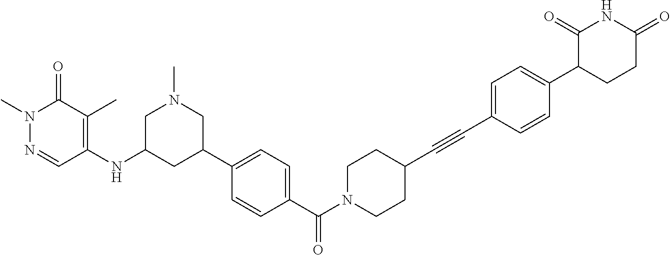 [SMILES: Cc1c(NC2CC(c3ccc(C(=O)N4CCC(C#Cc5ccc(C6CCC(=O)NC6=O)cc5)CC4)cc3)CN(C)C2)cnn(C)c1=O] |
| 334 | 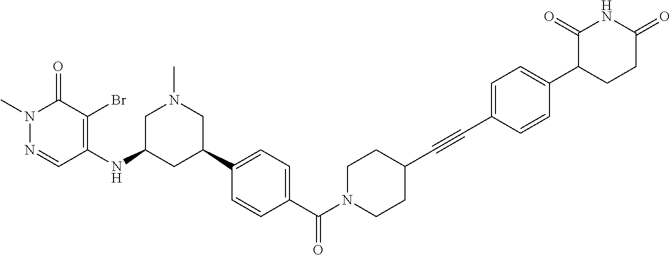 [SMILES: CN1C[C@H](Nc2cnn(C)c(=O)c2Br)C[C@H](c2ccc(C(=O)N3CCC(C#Cc4ccc(C5CCC(=O)NC5=O)cc4)CC3)cc2)C1] |
| 334a | 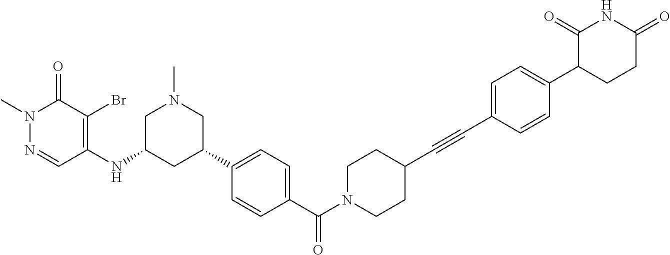 [SMILES: CN1C[C@@H](Nc2cnn(C)c(=O)c2Br)C[C@@H](c2ccc(C(=O)N3CCC(C#Cc4ccc(C5CCC(=O)NC5=O)cc4)CC3)cc2)C1] |
| 334b | 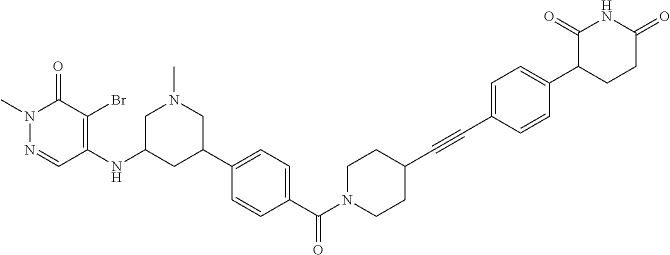 [SMILES: CN1CC(Nc2cnn(C)c(=O)c2Br)CC(c2ccc(C(=O)N3CCC(C#Cc4ccc(C5CCC(=O)NC5=O)cc4)CC3)cc2)C1] |
| 335 | 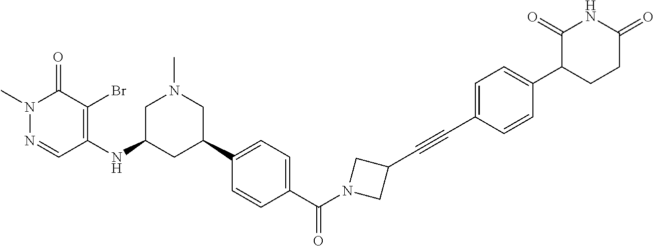 [SMILES: CN1C[C@H](Nc2cnn(C)c(=O)c2Br)C[C@H](c2ccc(C(=O)N3CC(C#Cc4ccc(C5CCC(=O)NC5=O)cc4)C3)cc2)C1] |
| 335a | 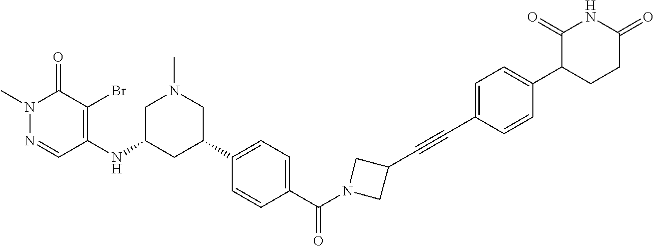 [SMILES: CN1C[C@@H](Nc2cnn(C)c(=O)c2Br)C[C@@H](c2ccc(C(=O)N3CC(C#Cc4ccc(C5CCC(=O)NC5=O)cc4)C3)cc2)C1] |
| 335b | 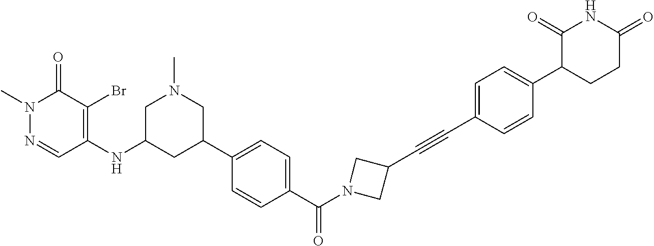 [SMILES: CN1CC(Nc2cnn(C)c(=O)c2Br)CC(c2ccc(C(=O)N3CC(C#Cc4ccc(C5CCC(=O)NC5=O)cc4)C3)cc2)C1] |
| 336 | 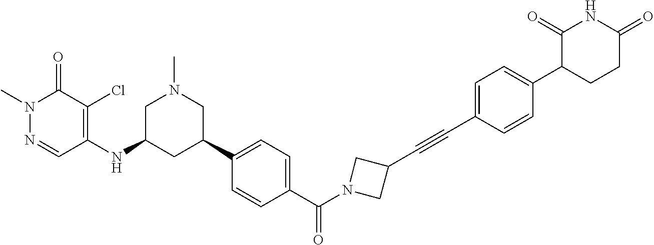 [SMILES: CN1C[C@H](Nc2cnn(C)c(=O)c2Cl)C[C@H](c2ccc(C(=O)N3CC(C#Cc4ccc(C5CCC(=O)NC5=O)cc4)C3)cc2)C1] |
| 336a | 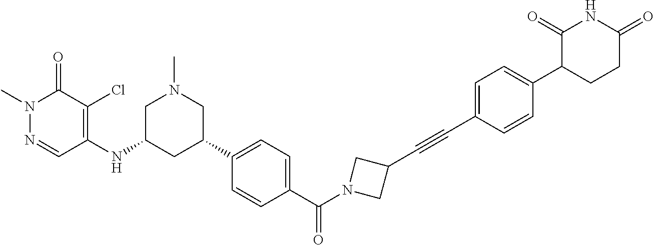 [SMILES: CN1C[C@@H](Nc2cnn(C)c(=O)c2Cl)C[C@@H](c2ccc(C(=O)N3CC(C#Cc4ccc(C5CCC(=O)NC5=O)cc4)C3)cc2)C1] |
| 336b | 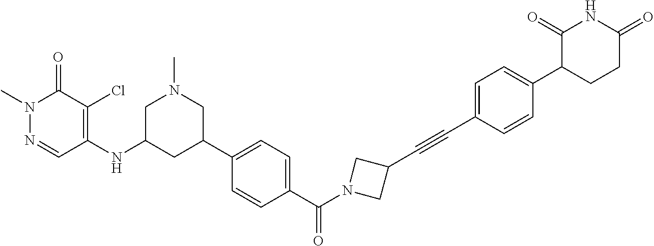 [SMILES: CN1CC(Nc2cnn(C)c(=O)c2Cl)CC(c2ccc(C(=O)N3CC(C#Cc4ccc(C5CCC(=O)NC5=O)cc4)C3)cc2)C1] |
| 337 | 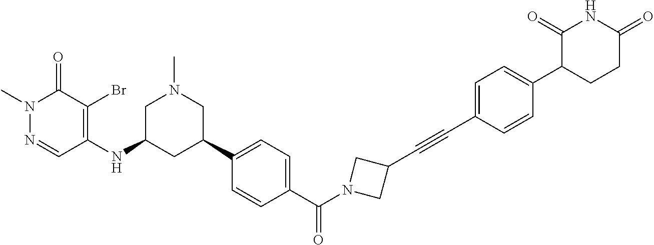 [SMILES: CN1C[C@H](Nc2cnn(C)c(=O)c2Br)C[C@H](c2ccc(C(=O)N3CC(C#Cc4ccc(C5CCC(=O)NC5=O)cc4)C3)cc2)C1] |
| 337a | 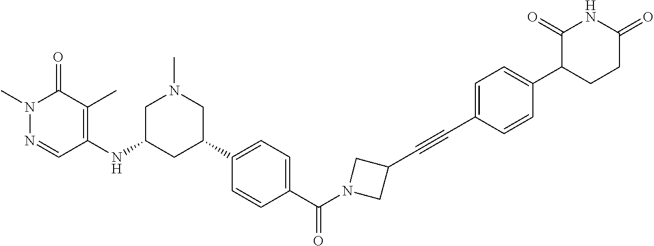 [SMILES: Cc1c(N[C@H]2C[C@@H](c3ccc(C(=O)N4CC(C#Cc5ccc(C6CCC(=O)NC6=O)cc5)C4)cc3)CN(C)C2)cnn(C)c1=O] |
| 337b | 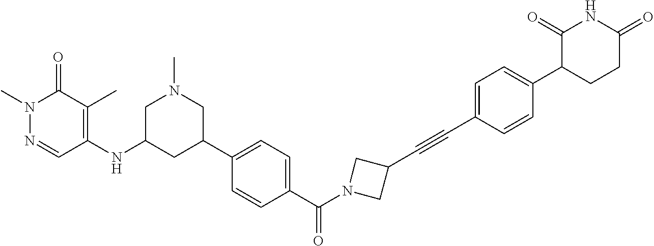 [SMILES: Cc1c(NC2CC(c3ccc(C(=O)N4CC(C#Cc5ccc(C6CCC(=O)NC6=O)cc5)C4)cc3)CN(C)C2)cnn(C)c1=O] |
| 338 | 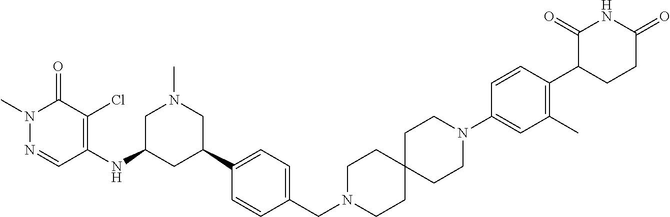 [SMILES: Cc1cc(N2CCC3(CCN(Cc4ccc([C@H]5C[C@@H](Nc6cnn(C)c(=O)c6Cl)CN(C)C5)cc4)CC3)CC2)ccc1C1CCC(=O)NC1=O] |
| 338a | 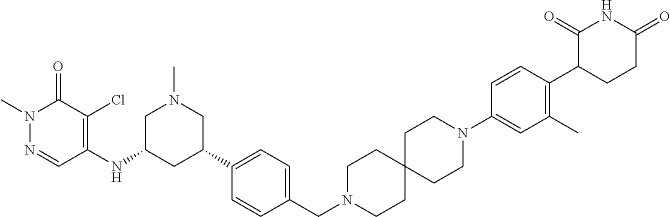 [SMILES: Cc1cc(N2CCC3(CCN(Cc4ccc([C@@H]5C[C@H](Nc6cnn(C)c(=O)c6Cl)CN(C)C5)cc4)CC3)CC2)ccc1C1CCC(=O)NC1=O] |
| 338b | 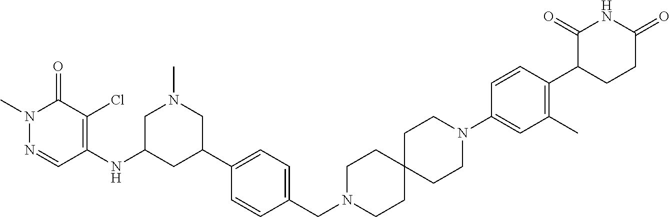 [SMILES: Cc1cc(N2CCC3(CCN(Cc4ccc(C5CC(Nc6cnn(C)c(=O)c6Cl)CN(C)C5)cc4)CC3)CC2)ccc1C1CCC(=O)NC1=O] |
| 339 | 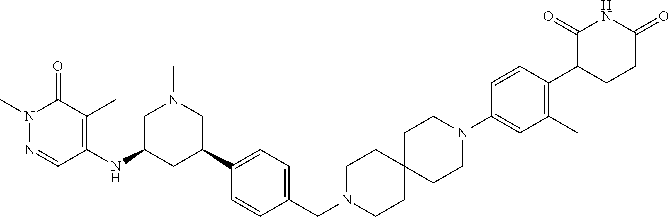 [SMILES: Cc1cc(N2CCC3(CCN(Cc4ccc([C@H]5C[C@@H](Nc6cnn(C)c(=O)c6C)CN(C)C5)cc4)CC3)CC2)ccc1C1CCC(=O)NC1=O] |
| 339a | 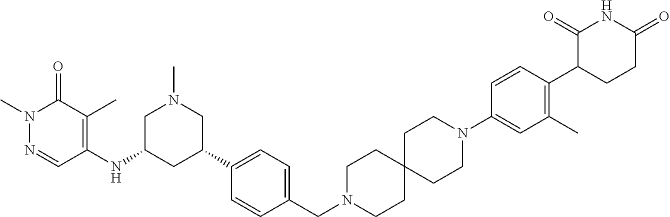 [SMILES: Cc1cc(N2CCC3(CCN(Cc4ccc([C@@H]5C[C@H](Nc6cnn(C)c(=O)c6C)CN(C)C5)cc4)CC3)CC2)ccc1C1CCC(=O)NC1=O] |
| 339b | 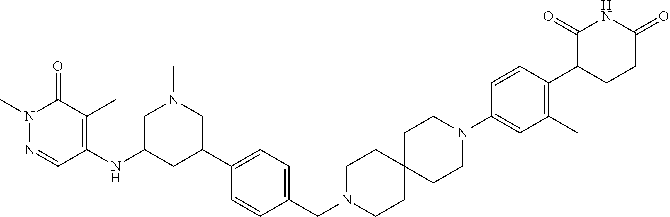 [SMILES: Cc1cc(N2CCC3(CCN(Cc4ccc(C5CC(Nc6cnn(C)c(=O)c6C)CN(C)C5)cc4)CC3)CC2)ccc1C1CCC(=O)NC1=O] |
| 340 | 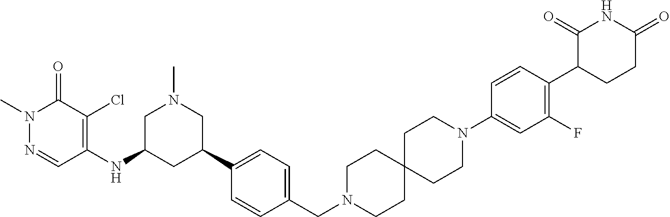 [SMILES: CN1C[C@H](Nc2cnn(C)c(=O)c2Cl)C[C@H](c2ccc(CN3CCC4(CC3)CCN(c3ccc(C5CCC(=O)NC5=O)c(F)c3)CC4)cc2)C1] |
| 340a | 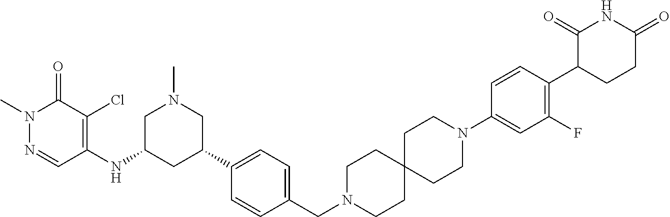 [SMILES: CN1C[C@@H](Nc2cnn(C)c(=O)c2Cl)C[C@@H](c2ccc(CN3CCC4(CC3)CCN(c3ccc(C5CCC(=O)NC5=O)c(F)c3)CC4)cc2)C1] |
| 340b | 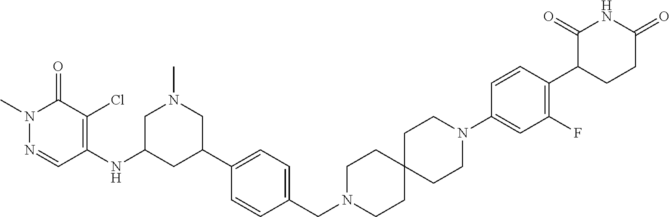 [SMILES: CN1CC(Nc2cnn(C)c(=O)c2Cl)CC(c2ccc(CN3CCC4(CC3)CCN(c3ccc(C5CCC(=O)NC5=O)c(F)c3)CC4)cc2)C1] |
| 341 | 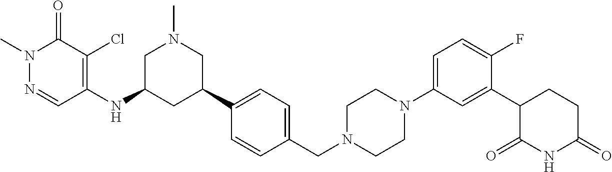 [SMILES: CN1C[C@H](Nc2cnn(C)c(=O)c2Cl)C[C@H](c2ccc(CN3CCN(c4ccc(F)c(C5CCC(=O)NC5=O)c4)CC3)cc2)C1] |
| 341a | 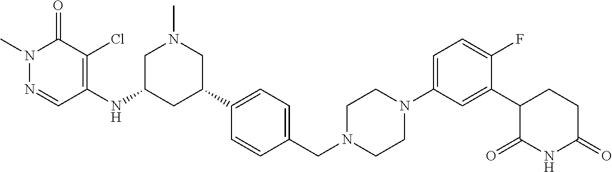 [SMILES: CN1C[C@@H](Nc2cnn(C)c(=O)c2Cl)C[C@@H](c2ccc(CN3CCN(c4ccc(F)c(C5CCC(=O)NC5=O)c4)CC3)cc2)C1] |
| 341b | 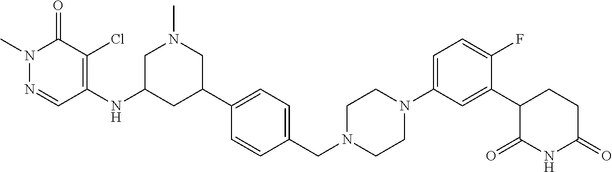 [SMILES: CN1CC(Nc2cnn(C)c(=O)c2Cl)CC(c2ccc(CN3CCN(c4ccc(F)c(C5CCC(=O)NC5=O)c4)CC3)cc2)C1] |
| 342 | 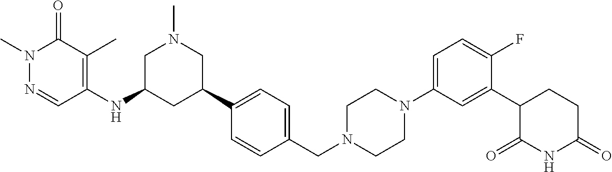 [SMILES: Cc1c(N[C@@H]2C[C@H](c3ccc(CN4CCN(c5ccc(F)c(C6CCC(=O)NC6=O)c5)CC4)cc3)CN(C)C2)cnn(C)c1=O] |
| 342a | 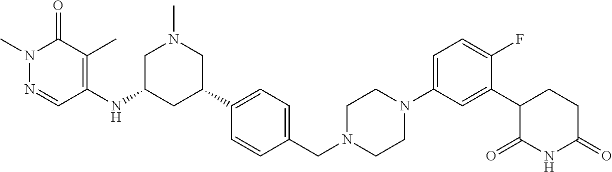 [SMILES: Cc1c(N[C@H]2C[C@@H](c3ccc(CN4CCN(c5ccc(F)c(C6CCC(=O)NC6=O)c5)CC4)cc3)CN(C)C2)cnn(C)c1=O] |
| 342b | 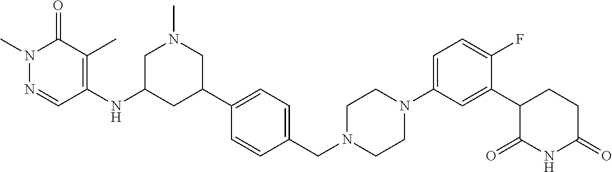 [SMILES: Cc1c(NC2CC(c3ccc(CN4CCN(c5ccc(F)c(C6CCC(=O)NC6=O)c5)CC4)cc3)CN(C)C2)cnn(C)c1=O] |
| 343 | 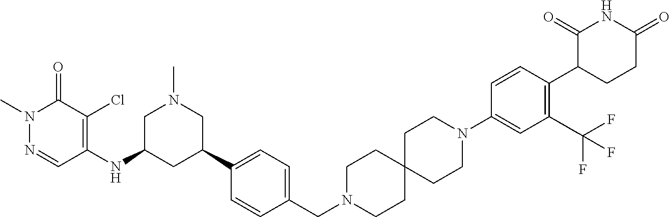 |
| 343a | 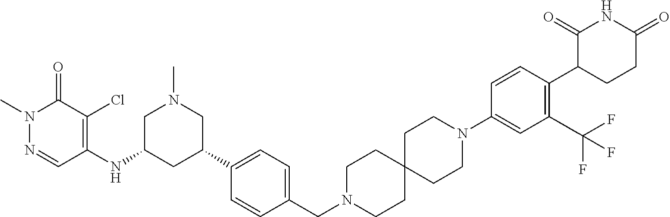 |
| 343b | 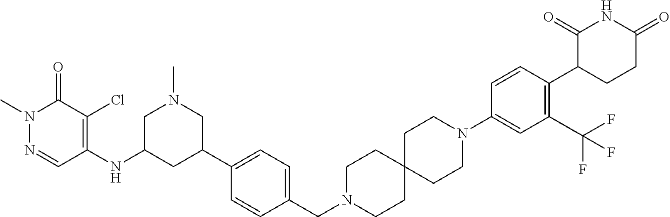 |
| 344 | 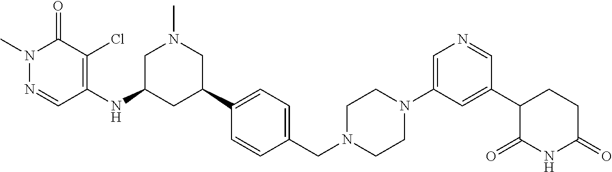 [SMILES: CN1C[C@H](Nc2cnn(C)c(=O)c2Cl)C[C@H](c2ccc(CN3CCN(c4cncc(C5CCC(=O)NC5=O)c4)CC3)cc2)C1] |
| 344a | 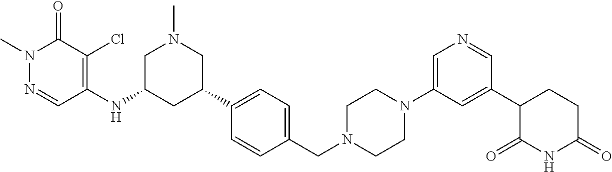 [SMILES: CN1C[C@@H](Nc2cnn(C)c(=O)c2Cl)C[C@@H](c2ccc(CN3CCN(c4cncc(C5CCC(=O)NC5=O)c4)CC3)cc2)C1] |
| 344b | 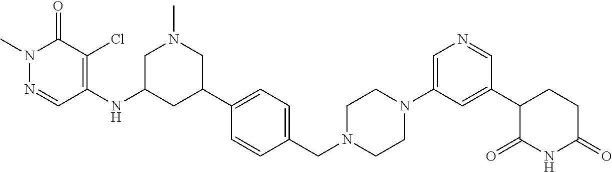 [SMILES: CN1CC(Nc2cnn(C)c(=O)c2Cl)CC(c2ccc(CN3CCN(c4cncc(C5CCC(=O)NC5=O)c4)CC3)cc2)C1] |
| 345 | 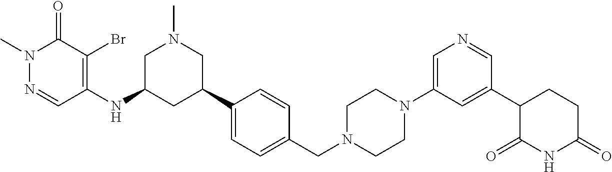 [SMILES: CN1C[C@H](Nc2cnn(C)c(=O)c2Br)C[C@H](c2ccc(CN3CCN(c4cncc(C5CCC(=O)NC5=O)c4)CC3)cc2)C1] |
| 345a | 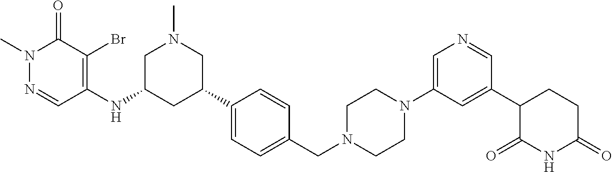 [SMILES: CN1C[C@@H](Nc2cnn(C)c(=O)c2Br)C[C@@H](c2ccc(CN3CCN(c4cncc(C5CCC(=O)NC5=O)c4)CC3)cc2)C1] |
| 345b | 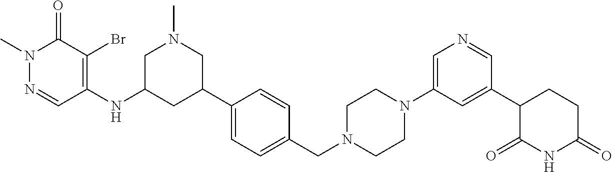 [SMILES: CN1CC(Nc2cnn(C)c(=O)c2Br)CC(c2ccc(CN3CCN(c4cncc(C5CCC(=O)NC5=O)c4)CC3)cc2)C1] |
| 346 | 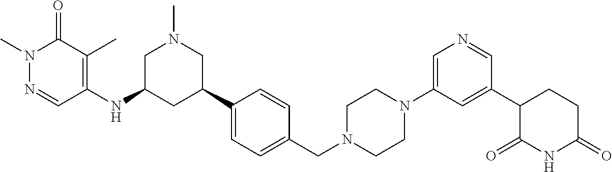 [SMILES: Cc1c(N[C@@H]2C[C@H](c3ccc(CN4CCN(c5cncc(C6CCC(=O)NC6=O)c5)CC4)cc3)CN(C)C2)cnn(C)c1=O] |
| 346a | 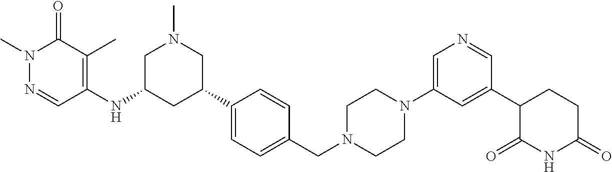 [SMILES: Cc1c(N[C@H]2C[C@@H](c3ccc(CN4CCN(c5cncc(C6CCC(=O)NC6=O)c5)CC4)cc3)CN(C)C2)cnn(C)c1=O] |
| 346b | 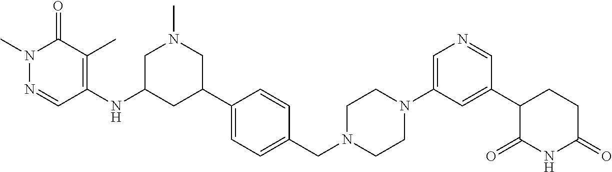 [SMILES: Cc1c(NC2CC(c3ccc(CN4CCN(c5cncc(C6CCC(=O)NC6=O)c5)CC4)cc3)CN(C)C2)cnn(C)c1=O] |
| 347 | 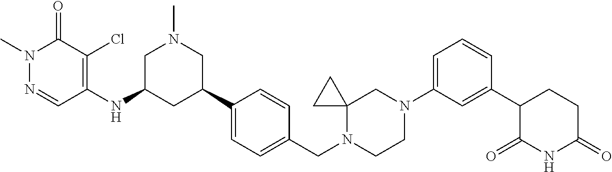 [SMILES: CN1C[C@H](Nc2cnn(C)c(=O)c2Cl)C[C@H](c2ccc(CN3CCN(c4cccc(C5CCC(=O)NC5=O)c4)CC34CC4)cc2)C1] |
| 347a | 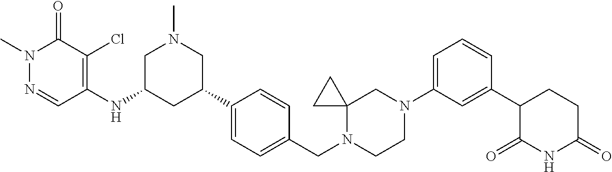 [SMILES: CN1C[C@@H](Nc2cnn(C)c(=O)c2Cl)C[C@@H](c2ccc(CN3CCN(c4cccc(C5CCC(=O)NC5=O)c4)CC34CC4)cc2)C1] |
| 347b | 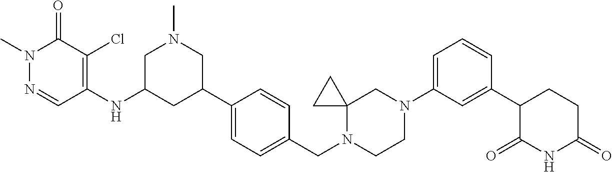 [SMILES: CN1CC(Nc2cnn(C)c(=O)c2Cl)CC(c2ccc(CN3CCN(c4cccc(C5CCC(=O)NC5=O)c4)CC34CC4)cc2)C1] |
| 348 | 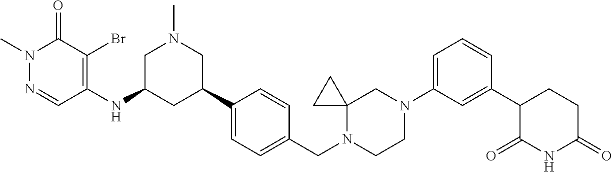 [SMILES: CN1C[C@H](Nc2cnn(C)c(=O)c2Br)C[C@H](c2ccc(CN3CCN(c4cccc(C5CCC(=O)NC5=O)c4)CC34CC4)cc2)C1] |
| 348a | 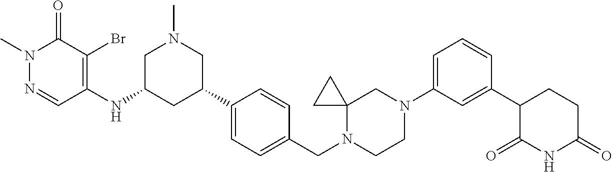 [SMILES: CN1C[C@@H](Nc2cnn(C)c(=O)c2Br)C[C@@H](c2ccc(CN3CCN(c4cccc(C5CCC(=O)NC5=O)c4)CC34CC4)cc2)C1] |
| 348b | 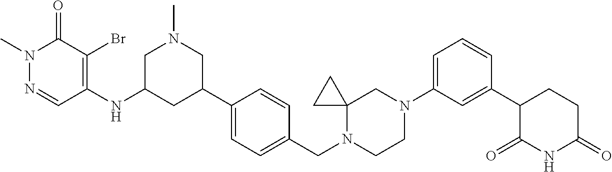 [SMILES: CN1CC(Nc2cnn(C)c(=O)c2Br)CC(c2ccc(CN3CCN(c4cccc(C5CCC(=O)NC5=O)c4)CC34CC4)cc2)C1] |
| 349 | 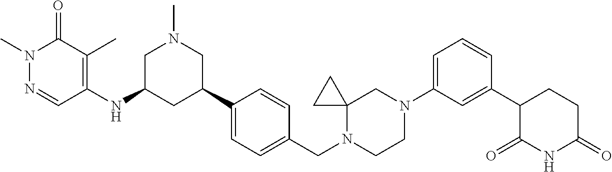 [SMILES: Cc1c(N[C@@H]2C[C@H](c3ccc(CN4CCN(c5cccc(C6CCC(=O)NC6=O)c5)CC45CC5)cc3)CN(C)C2)cnn(C)c1=O] |
| 349a | 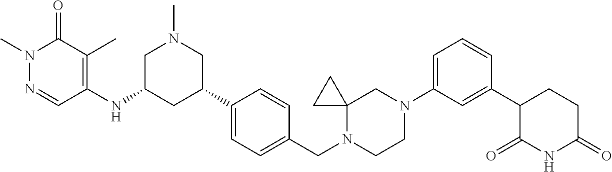 [SMILES: Cc1c(N[C@H]2C[C@@H](c3ccc(CN4CCN(c5cccc(C6CCC(=O)NC6=O)c5)CC45CC5)cc3)CN(C)C2)cnn(C)c1=O] |
| 349b | 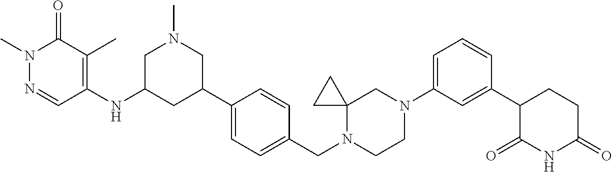 [SMILES: Cc1c(NC2CC(c3ccc(CN4CCN(c5cccc(C6CCC(=O)NC6=O)c5)CC45CC5)cc3)CN(C)C2)cnn(C)c1=O] |
| 350 | 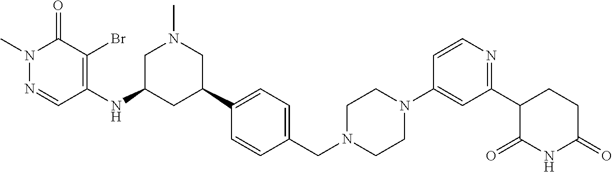 [SMILES: CN1C[C@H](Nc2cnn(C)c(=O)c2Br)C[C@H](c2ccc(CN3CCN(c4ccnc(C5CCC(=O)NC5=O)c4)CC3)cc2)C1] |
| 350a | 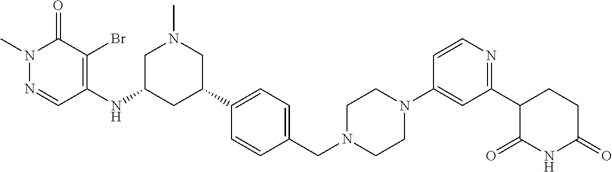 [SMILES: CN1C[C@@H](Nc2cnn(C)c(=O)c2Br)C[C@@H](c2ccc(CN3CCN(c4ccnc(C5CCC(=O)NC5=O)c4)CC3)cc2)C1] |
| 350b | 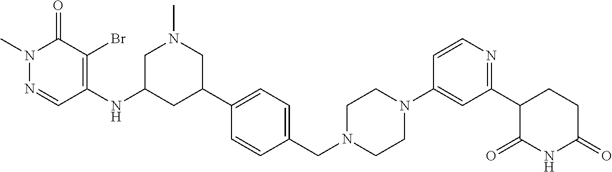 [SMILES: CN1CC(Nc2cnn(C)c(=O)c2Br)CC(c2ccc(CN3CCN(c4ccnc(C5CCC(=O)NC5=O)c4)CC3)cc2)C1] |
| 351 | 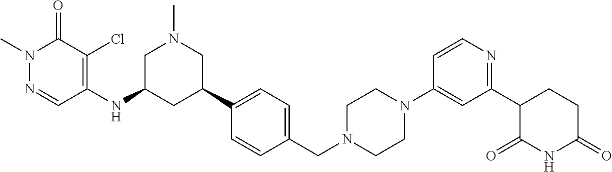 [SMILES: CN1C[C@H](Nc2cnn(C)c(=O)c2Cl)C[C@H](c2ccc(CN3CCN(c4ccnc(C5CCC(=O)NC5=O)c4)CC3)cc2)C1] |
| 351a | 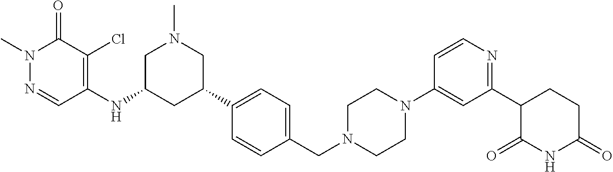 [SMILES: CN1C[C@@H](Nc2cnn(C)c(=O)c2Cl)C[C@@H](c2ccc(CN3CCN(c4ccnc(C5CCC(=O)NC5=O)c4)CC3)cc2)C1] |
| 351b | 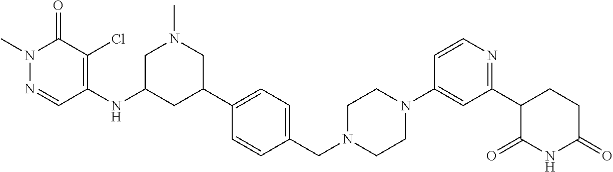 [SMILES: CN1CC(Nc2cnn(C)c(=O)c2Cl)CC(c2ccc(CN3CCN(c4ccnc(C5CCC(=O)NC5=O)c4)CC3)cc2)C1] |
| 352 | 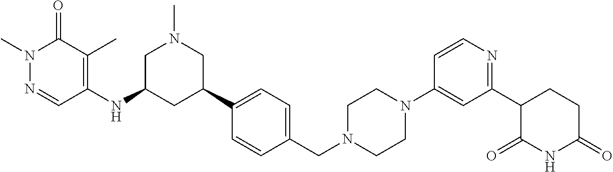 [SMILES: Cc1c(N[C@@H]2C[C@H](c3ccc(CN4CCN(c5ccnc(C6CCC(=O)NC6=O)c5)CC4)cc3)CN(C)C2)cnn(C)c1=O] |
| 352a | 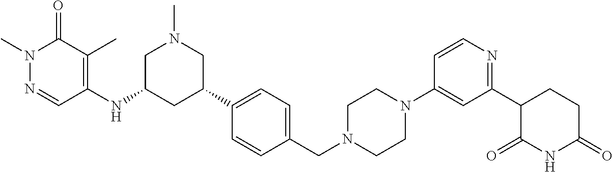 [SMILES: Cc1c(N[C@H]2C[C@@H](c3ccc(CN4CCN(c5ccnc(C6CCC(=O)NC6=O)c5)CC4)cc3)CN(C)C2)cnn(C)c1=O] |
| 352b | 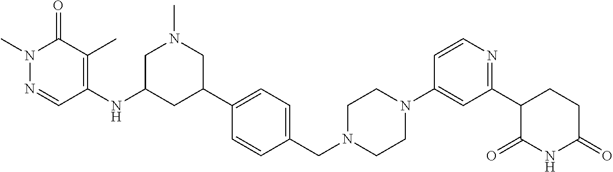 [SMILES: Cc1c(NC2CC(c3ccc(CN4CCN(c5ccnc(C6CCC(=O)NC6=O)c5)CC4)cc3)CN(C)C2)cnn(C)c1=O] |
| 353 | 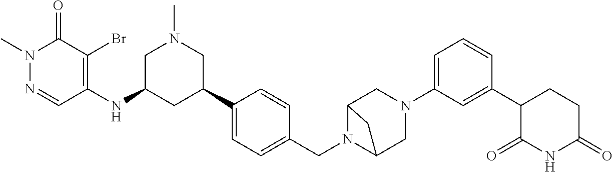 [SMILES: CN1C[C@H](Nc2cnn(C)c(=O)c2Br)C[C@H](c2ccc(CN3C4CC3CN(c3cccc(C5CCC(=O)NC5=O)c3)C4)cc2)C1] |
| 353a | 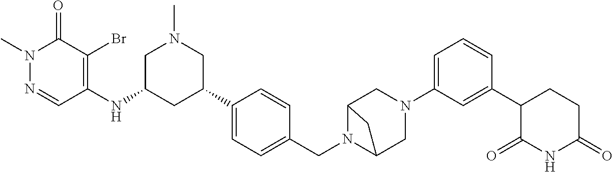 [SMILES: CN1C[C@@H](Nc2cnn(C)c(=O)c2Br)C[C@@H](c2ccc(CN3C4CC3CN(c3cccc(C5CCC(=O)NC5=O)c3)C4)cc2)C1] |
| 353b | 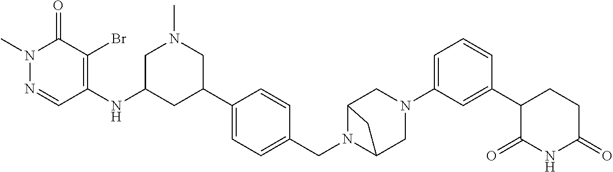 [SMILES: CN1CC(Nc2cnn(C)c(=O)c2Br)CC(c2ccc(CN3C4CC3CN(c3cccc(C5CCC(=O)NC5=O)c3)C4)cc2)C1] |
| 354 | 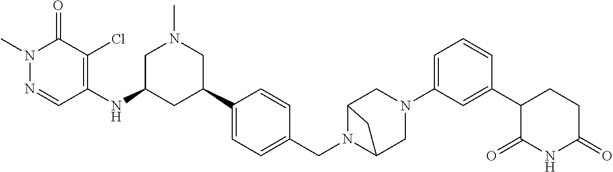 [SMILES: CN1C[C@H](Nc2cnn(C)c(=O)c2Cl)C[C@H](c2ccc(CN3C4CC3CN(c3cccc(C5CCC(=O)NC5=O)c3)C4)cc2)C1] |
| 354a | 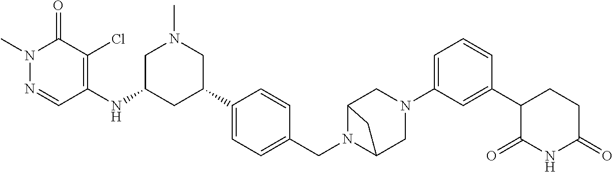 [SMILES: CN1C[C@@H](Nc2cnn(C)c(=O)c2Cl)C[C@@H](c2ccc(CN3C4CC3CN(c3cccc(C5CCC(=O)NC5=O)c3)C4)cc2)C1] |
| 354b | 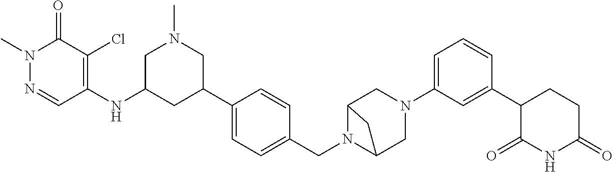 [SMILES: CN1CC(Nc2cnn(C)c(=O)c2Cl)CC(c2ccc(CN3C4CC3CN(c3cccc(C5CCC(=O)NC5=O)c3)C4)cc2)C1] |
| 355 | 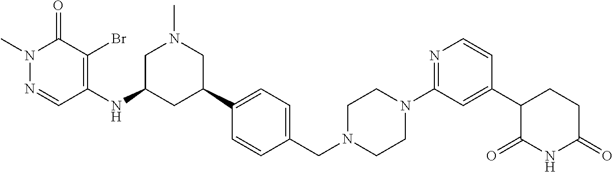 [SMILES: CN1C[C@H](Nc2cnn(C)c(=O)c2Br)C[C@H](c2ccc(CN3CCN(c4cc(C5CCC(=O)NC5=O)ccn4)CC3)cc2)C1] |
| 355a | 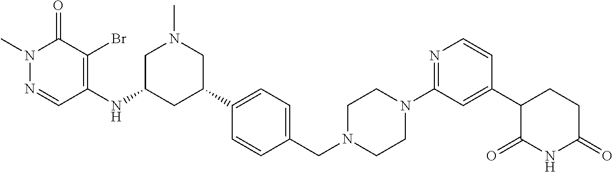 [SMILES: CN1C[C@@H](Nc2cnn(C)c(=O)c2Br)C[C@@H](c2ccc(CN3CCN(c4cc(C5CCC(=O)NC5=O)ccn4)CC3)cc2)C1] |
| 355b | 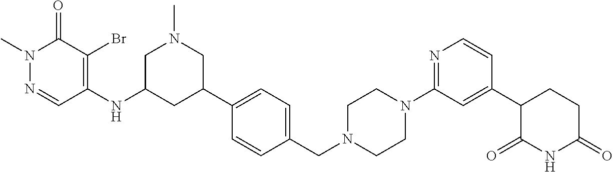 [SMILES: CN1CC(Nc2cnn(C)c(=O)c2Br)CC(c2ccc(CN3CCN(c4cc(C5CCC(=O)NC5=O)ccn4)CC3)cc2)C1] |
| 356 | 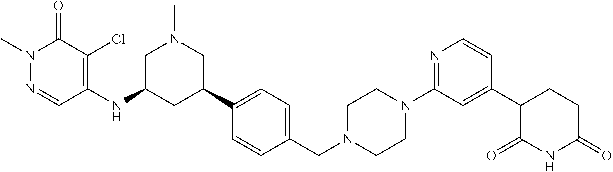 [SMILES: CN1C[C@H](Nc2cnn(C)c(=O)c2Cl)C[C@H](c2ccc(CN3CCN(c4cc(C5CCC(=O)NC5=O)ccn4)CC3)cc2)C1] |
| 356a | 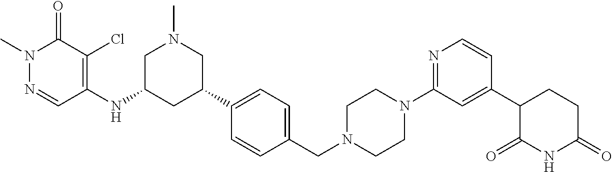 [SMILES: CN1C[C@@H](Nc2cnn(C)c(=O)c2Cl)C[C@@H](c2ccc(CN3CCN(c4cc(C5CCC(=O)NC5=O)ccn4)CC3)cc2)C1] |
| 356b | 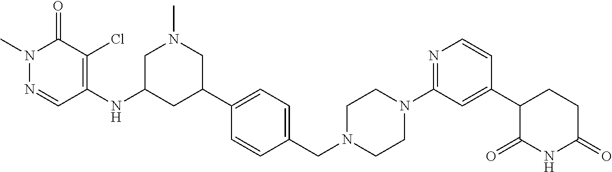 [SMILES: CN1CC(Nc2cnn(C)c(=O)c2Cl)CC(c2ccc(CN3CCN(c4cc(C5CCC(=O)NC5=O)ccn4)CC3)cc2)C1] |
| 357 | 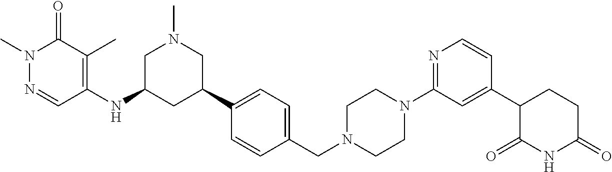 [SMILES: Cc1c(N[C@@H]2C[C@H](c3ccc(CN4CCN(c5cc(C6CCC(=O)NC6=O)ccn5)CC4)cc3)CN(C)C2)cnn(C)c1=O] |
| 357a | 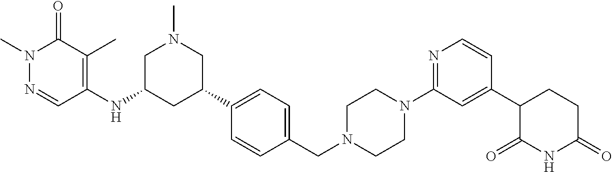 [SMILES: Cc1c(N[C@H]2C[C@@H](c3ccc(CN4CCN(c5cc(C6CCC(=O)NC6=O)ccn5)CC4)cc3)CN(C)C2)cnn(C)c1=O] |
| 357b | 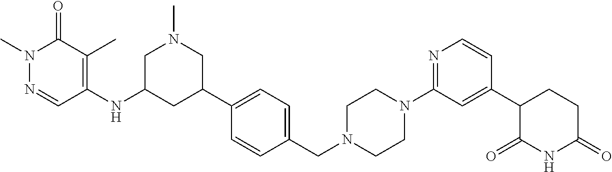 [SMILES: Cc1c(NC2CC(c3ccc(CN4CCN(c5cc(C6CCC(=O)NC6=O)ccn5)CC4)cc3)CN(C)C2)cnn(C)c1=O] |
| 358 | 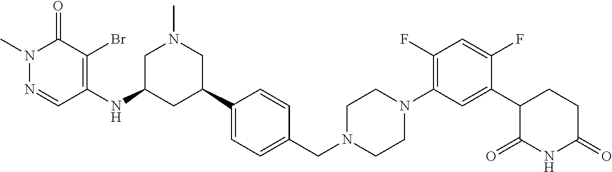 [SMILES: CN1C[C@H](Nc2cnn(C)c(=O)c2Br)C[C@H](c2ccc(CN3CCN(c4cc(C5CCC(=O)NC5=O)c(F)cc4F)CC3)cc2)C1] |
| 358a | 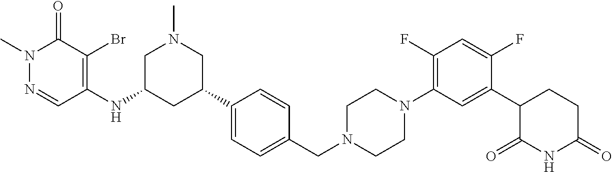 [SMILES: CN1C[C@@H](Nc2cnn(C)c(=O)c2Br)C[C@@H](c2ccc(CN3CCN(c4cc(C5CCC(=O)NC5=O)c(F)cc4F)CC3)cc2)C1] |
| 358b | 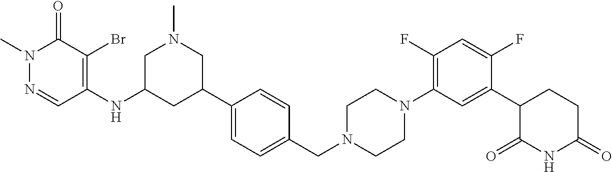 [SMILES: CN1CC(Nc2cnn(C)c(=O)c2Br)CC(c2ccc(CN3CCN(c4cc(C5CCC(=O)NC5=O)c(F)cc4F)CC3)cc2)C1] |
| 359 | 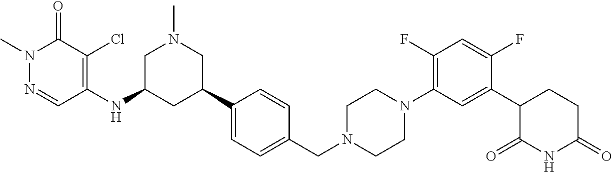 [SMILES: CN1C[C@H](Nc2cnn(C)c(=O)c2Cl)C[C@H](c2ccc(CN3CCN(c4cc(C5CCC(=O)NC5=O)c(F)cc4F)CC3)cc2)C1] |
| 359a | 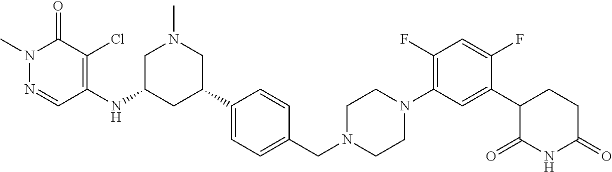 [SMILES: CN1C[C@@H](Nc2cnn(C)c(=O)c2Cl)C[C@@H](c2ccc(CN3CCN(c4cc(C5CCC(=O)NC5=O)c(F)cc4F)CC3)cc2)C1] |
| 359b | 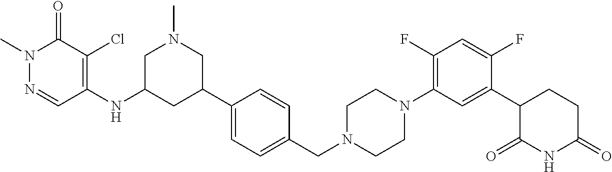 [SMILES: CN1CC(Nc2cnn(C)c(=O)c2Cl)CC(c2ccc(CN3CCN(c4cc(C5CCC(=O)NC5=O)c(F)cc4F)CC3)cc2)C1] |
| 360 | 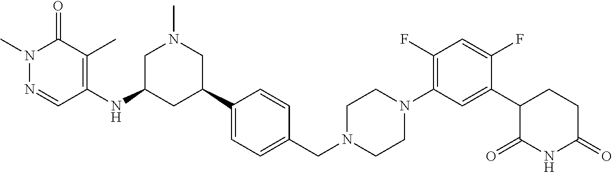 [SMILES: Cc1c(N[C@@H]2C[C@H](c3ccc(CN4CCN(c5cc(C6CCC(=O)NC6=O)c(F)cc5F)CC4)cc3)CN(C)C2)cnn(C)c1=O] |
| 360a | 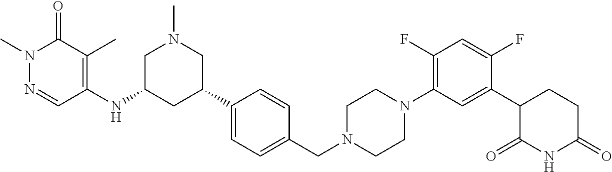 [SMILES: Cc1c(N[C@H]2C[C@@H](c3ccc(CN4CCN(c5cc(C6CCC(=O)NC6=O)c(F)cc5F)CC4)cc3)CN(C)C2)cnn(C)c1=O] |
| 360b | 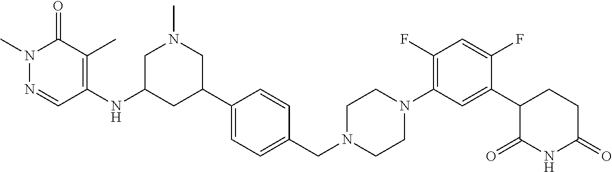 [SMILES: Cc1c(NC2CC(c3ccc(CN4CCN(c5cc(C6CCC(=O)NC6=O)c(F)cc5F)CC4)cc3)CN(C)C2)cnn(C)c1=O] |
| 361 | 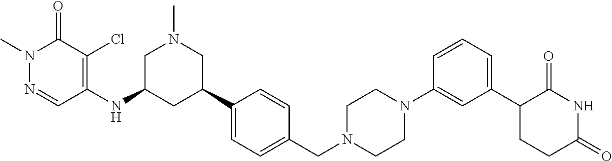 [SMILES: CN1C[C@H](Nc2cnn(C)c(=O)c2Cl)C[C@H](c2ccc(CN3CCN(c4cccc(C5CCC(=O)NC5=O)c4)CC3)cc2)C1] |
| 361a | 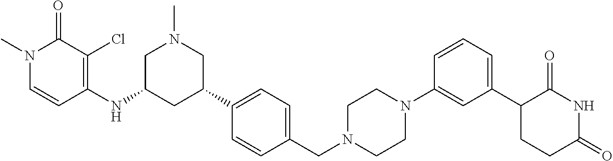 [SMILES: CN1C[C@@H](Nc2ccn(C)c(=O)c2Cl)C[C@@H](c2ccc(CN3CCN(c4cccc(C5CCC(=O)NC5=O)c4)CC3)cc2)C1] |
| 361b | 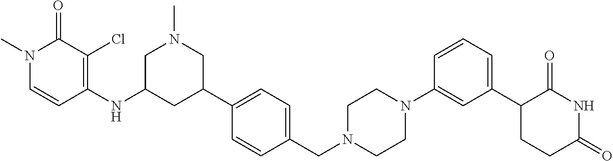 [SMILES: CN1CC(Nc2ccn(C)c(=O)c2Cl)CC(c2ccc(CN3CCN(c4cccc(C5CCC(=O)NC5=O)c4)CC3)cc2)C1] |
| 362 | 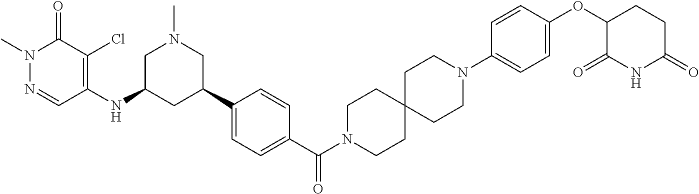 [SMILES: CN1C[C@H](Nc2cnn(C)c(=O)c2Cl)C[C@H](c2ccc(C(=O)N3CCC4(CC3)CCN(c3ccc(OC5CCC(=O)NC5=O)cc3)CC4)cc2)C1] |
| 362a | 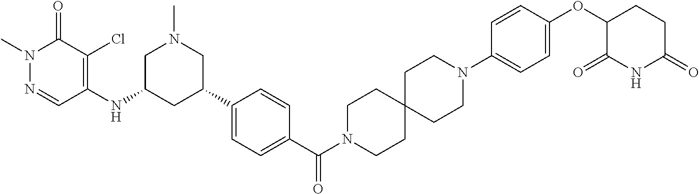 [SMILES: CN1C[C@@H](Nc2cnn(C)c(=O)c2Cl)C[C@@H](c2ccc(C(=O)N3CCC4(CC3)CCN(c3ccc(OC5CCC(=O)NC5=O)cc3)CC4)cc2)C1] |
| 362b | 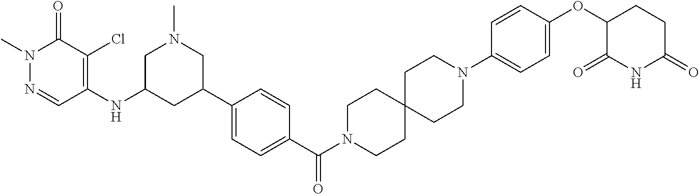 [SMILES: CN1CC(Nc2cnn(C)c(=O)c2Cl)CC(c2ccc(C(=O)N3CCC4(CC3)CCN(c3ccc(OC5CCC(=O)NC5=O)cc3)CC4)cc2)C1] |
| 363 | 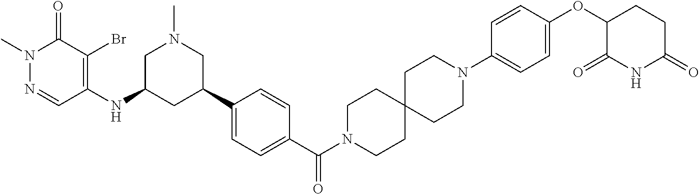 [SMILES: CN1C[C@H](Nc2cnn(C)c(=O)c2Br)C[C@H](c2ccc(C(=O)N3CCC4(CC3)CCN(c3ccc(OC5CCC(=O)NC5=O)cc3)CC4)cc2)C1] |
| 363a | 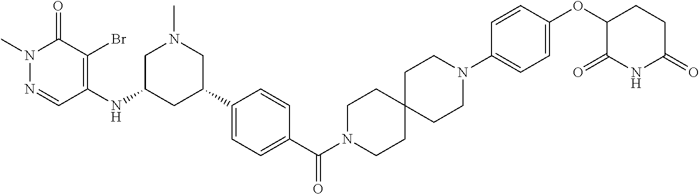 [SMILES: CN1C[C@@H](Nc2cnn(C)c(=O)c2Br)C[C@@H](c2ccc(C(=O)N3CCC4(CC3)CCN(c3ccc(OC5CCC(=O)NC5=O)cc3)CC4)cc2)C1] |
| 363b | 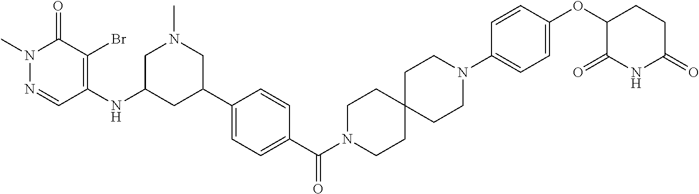 [SMILES: CN1CC(Nc2cnn(C)c(=O)c2Br)CC(c2ccc(C(=O)N3CCC4(CC3)CCN(c3ccc(OC5CCC(=O)NC5=O)cc3)CC4)cc2)C1] |
| 364 | 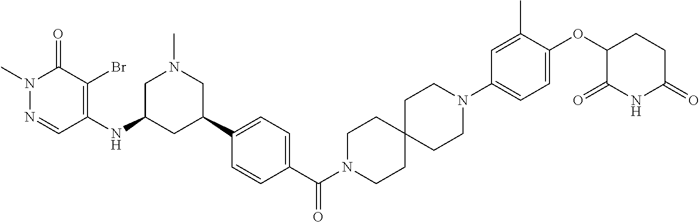 [SMILES: Cc1cc(N2CCC3(CCN(C(=O)c4ccc([C@H]5C[C@@H](Nc6cnn(C)c(=O)c6Br)CN(C)C5)cc4)CC3)CC2)ccc1OC1CCC(=O)NC1=O] |
| 364a | 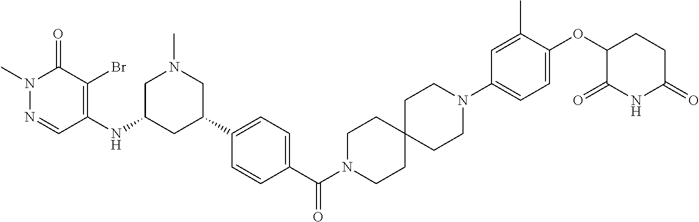 [SMILES: Cc1cc(N2CCC3(CCN(C(=O)c4ccc([C@@H]5C[C@H](Nc6cnn(C)c(=O)c6Br)CN(C)C5)cc4)CC3)CC2)ccc1OC1CCC(=O)NC1=O] |
| 364b | 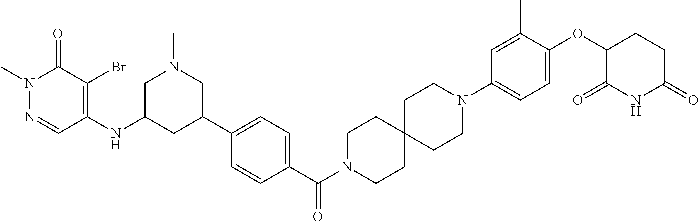 [SMILES: Cc1cc(N2CCC3(CCN(C(=O)c4ccc(C5CC(Nc6cnn(C)c(=O)c6Br)CN(C)C5)cc4)CC3)CC2)ccc1OC1CCC(=O)NC1=O] |
| 365 | 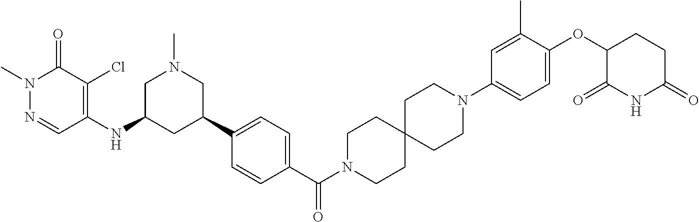 [SMILES: Cc1cc(N2CCC3(CCN(C(=O)c4ccc([C@H]5C[C@@H](Nc6cnn(C)c(=O)c6Cl)CN(C)C5)cc4)CC3)CC2)ccc1OC1CCC(=O)NC1=O] |
| 365a | 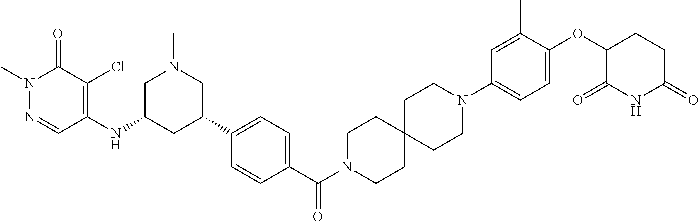 [SMILES: Cc1cc(N2CCC3(CCN(C(=O)c4ccc([C@@H]5C[C@H](Nc6cnn(C)c(=O)c6Cl)CN(C)C5)cc4)CC3)CC2)ccc1OC1CCC(=O)NC1=O] |
| 365b | 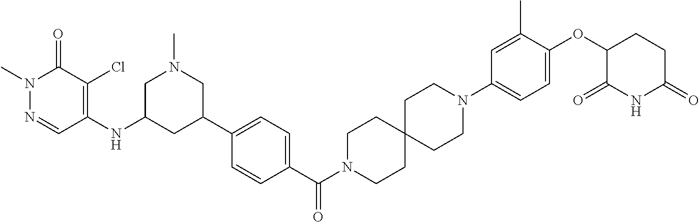 [SMILES: Cc1cc(N2CCC3(CCN(C(=O)c4ccc(C5CC(Nc6cnn(C)c(=O)c6Cl)CN(C)C5)cc4)CC3)CC2)ccc1OC1CCC(=O)NC1=O] |
| 366 | 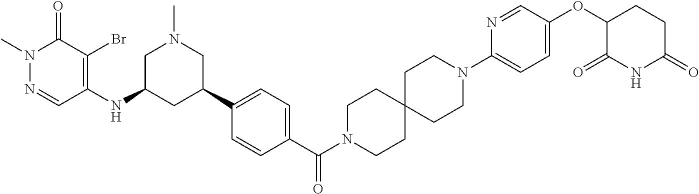 [SMILES: CN1C[C@H](Nc2cnn(C)c(=O)c2Br)C[C@H](c2ccc(C(=O)N3CCC4(CC3)CCN(c3ccc(OC5CCC(=O)NC5=O)cn3)CC4)cc2)C1] |
| 366a | 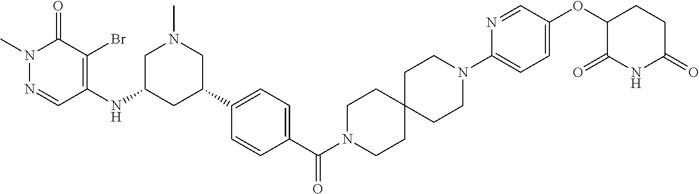 [SMILES: CN1C[C@@H](Nc2cnn(C)c(=O)c2Br)C[C@@H](c2ccc(C(=O)N3CCC4(CC3)CCN(c3ccc(OC5CCC(=O)NC5=O)cn3)CC4)cc2)C1] |
| 366b | 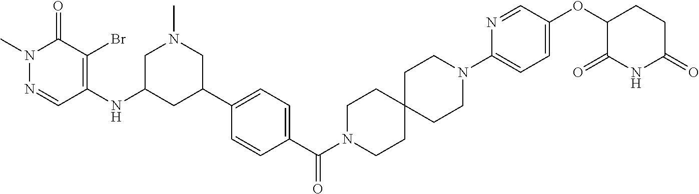 [SMILES: CN1CC(Nc2cnn(C)c(=O)c2Br)CC(c2ccc(C(=O)N3CCC4(CC3)CCN(c3ccc(OC5CCC(=O)NC5=O)cn3)CC4)cc2)C1] |
| 367 | 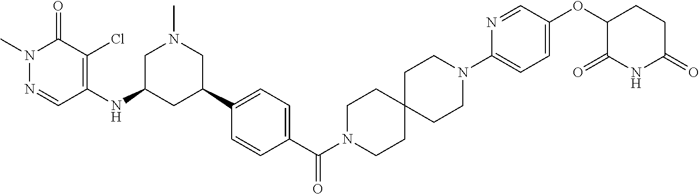 [SMILES: CN1C[C@H](Nc2cnn(C)c(=O)c2Cl)C[C@H](c2ccc(C(=O)N3CCC4(CC3)CCN(c3ccc(OC5CCC(=O)NC5=O)cn3)CC4)cc2)C1] |
| 367a | 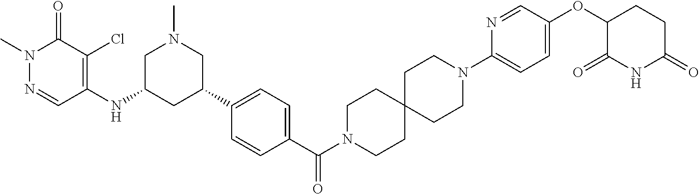 [SMILES: CN1C[C@@H](Nc2cnn(C)c(=O)c2Cl)C[C@@H](c2ccc(C(=O)N3CCC4(CC3)CCN(c3ccc(OC5CCC(=O)NC5=O)cn3)CC4)cc2)C1] |
| 367b | 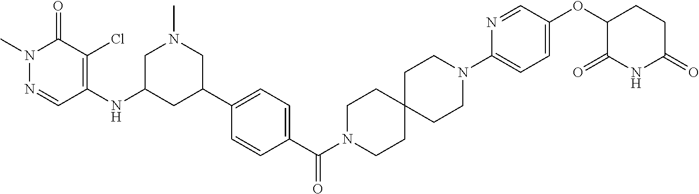 [SMILES: CN1CC(Nc2cnn(C)c(=O)c2Cl)CC(c2ccc(C(=O)N3CCC4(CC3)CCN(c3ccc(OC5CCC(=O)NC5=O)cn3)CC4)cc2)C1] |
| 368 | 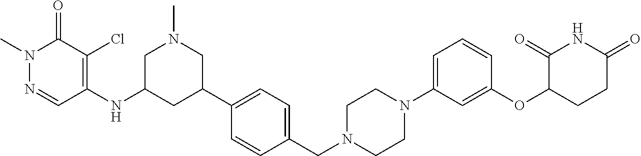 [SMILES: CN1CC(Nc2cnn(C)c(=O)c2Cl)CC(c2ccc(CN3CCN(c4cccc(OC5CCC(=O)NC5=O)c4)CC3)cc2)C1] |
| 368a | 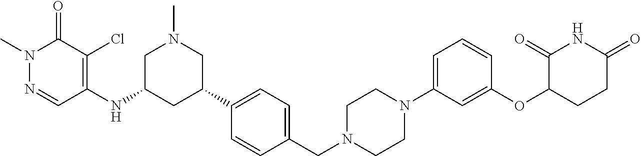 [SMILES: CN1C[C@@H](Nc2cnn(C)c(=O)c2Cl)C[C@@H](c2ccc(CN3CCN(c4cccc(OC5CCC(=O)NC5=O)c4)CC3)cc2)C1] |
| 368b | 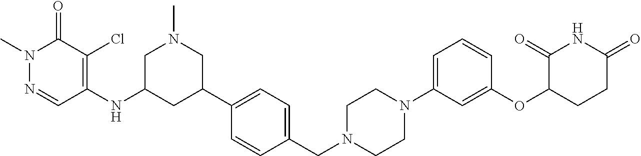 [SMILES: CN1CC(Nc2cnn(C)c(=O)c2Cl)CC(c2ccc(CN3CCN(c4cccc(OC5CCC(=O)NC5=O)c4)CC3)cc2)C1] |
| 369 | 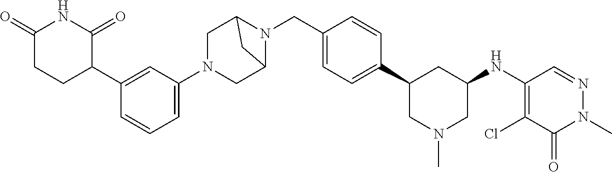 [SMILES: CN1C[C@H](Nc2cnn(C)c(=O)c2Cl)C[C@H](c2ccc(CN3C4CC3CN(c3cccc(C5CCC(=O)NC5=O)c3)C4)cc2)C1] |
| 369a | 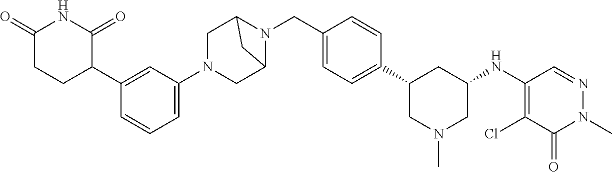 [SMILES: CN1C[C@@H](Nc2cnn(C)c(=O)c2Cl)C[C@@H](c2ccc(CN3C4CC3CN(c3cccc(C5CCC(=O)NC5=O)c3)C4)cc2)C1] |
| 369b | 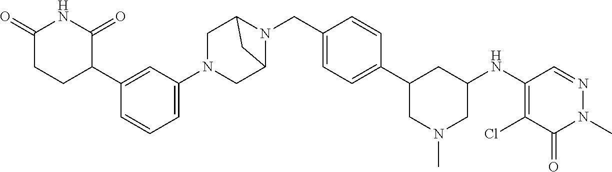 [SMILES: CN1CC(Nc2cnn(C)c(=O)c2Cl)CC(c2ccc(CN3C4CC3CN(c3cccc(C5CCC(=O)NC5=O)c3)C4)cc2)C1] |
| 370 | 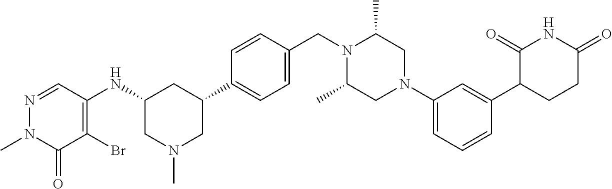 [SMILES: C[C@@H]1CN(c2cccc(C3CCC(=O)NC3=O)c2)C[C@H](C)N1Cc1ccc([C@H]2C[C@@H](Nc3cnn(C)c(=O)c3Br)CN(C)C2)cc1] |
| 370a | 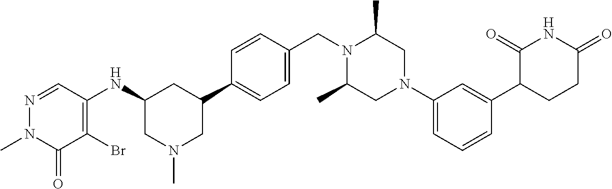 |
| 370b | 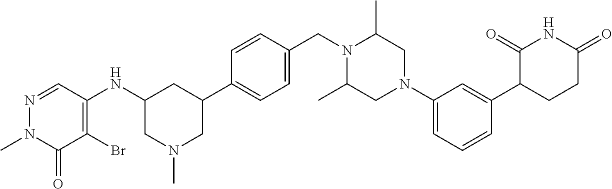 [SMILES: CC1CN(c2cccc(C3CCC(=O)NC3=O)c2)CC(C)N1Cc1ccc(C2CC(Nc3cnn(C)c(=O)c3Br)CN(C)C2)cc1] |
| 371 | 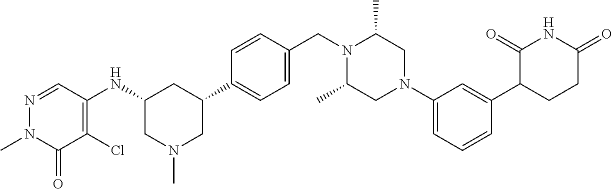 [SMILES: C[C@@H]1CN(c2cccc(C3CCC(=O)NC3=O)c2)C[C@H](C)N1Cc1ccc([C@H]2C[C@@H](Nc3cnn(C)c(=O)c3Cl)CN(C)C2)cc1] |
| 371a | 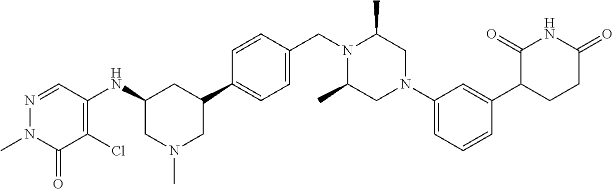 |
| 371b | 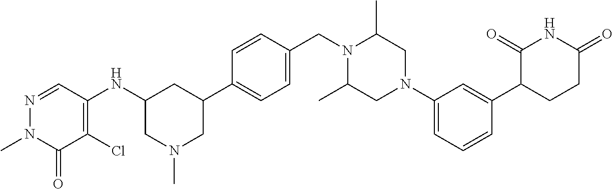 [SMILES: CC1CN(c2cccc(C3CCC(=O)NC3=O)c2)CC(C)N1Cc1ccc(C2CC(Nc3cnn(C)c(=O)c3Cl)CN(C)C2)cc1] |
| 372 | 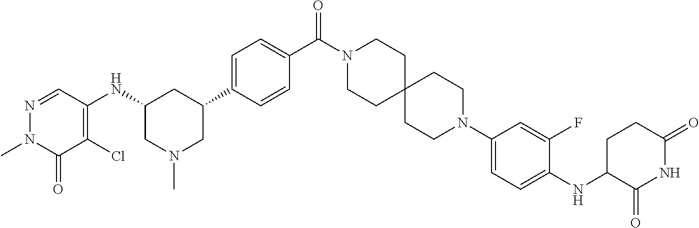 [SMILES: CN1C[C@H](Nc2cnn(C)c(=O)c2Cl)C[C@H](c2ccc(C(=O)N3CCC4(CC3)CCN(c3ccc(NC5CCC(=O)NC5=O)c(F)c3)CC4)cc2)C1] |
| 372a | 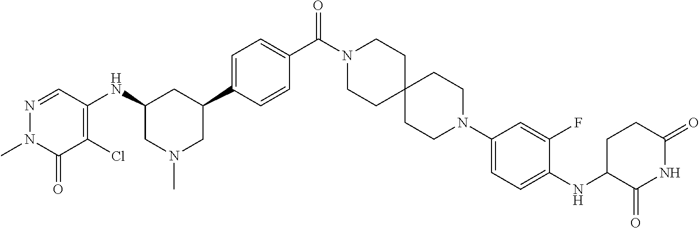 [SMILES: CN1C[C@@H](Nc2cnn(C)c(=O)c2Cl)C[C@@H](c2ccc(C(=O)N3CCC4(CC3)CCN(c3ccc(NC5CCC(=O)NC5=O)c(F)c3)CC4)cc2)C1] |
| 372b | 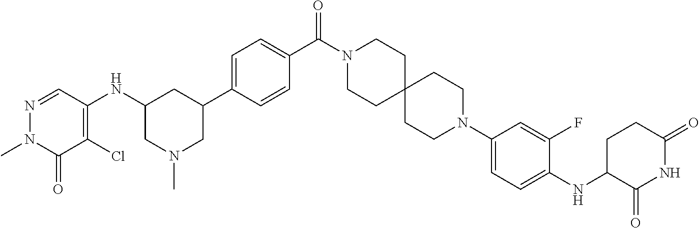 [SMILES: CN1CC(Nc2cnn(C)c(=O)c2Cl)CC(c2ccc(C(=O)N3CCC4(CC3)CCN(c3ccc(NC5CCC(=O)NC5=O)c(F)c3)CC4)cc2)C1] |
| 373 | 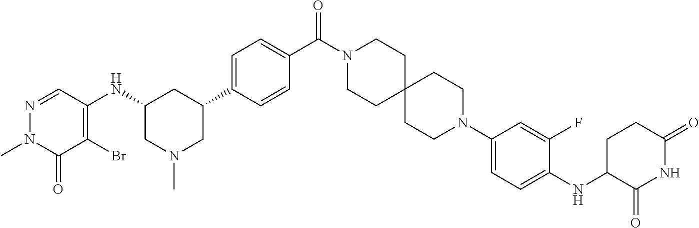 [SMILES: CN1C[C@H](Nc2cnn(C)c(=O)c2Br)C[C@H](c2ccc(C(=O)N3CCC4(CC3)CCN(c3ccc(NC5CCC(=O)NC5=O)c(F)c3)CC4)cc2)C1] |
| 373-1 | 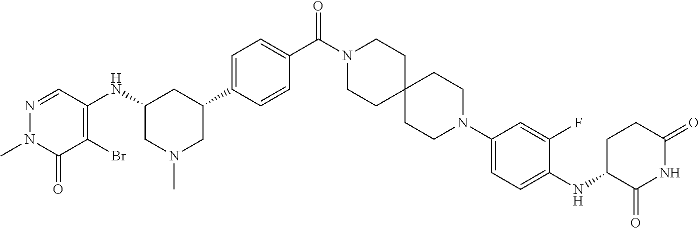 [SMILES: CN1C[C@H](Nc2cnn(C)c(=O)c2Br)C[C@H](c2ccc(C(=O)N3CCC4(CC3)CCN(c3ccc(N[C@@H]5CCC(=O)NC5=O)c(F)c3)CC4)cc2)C1] |
| 373-2 | 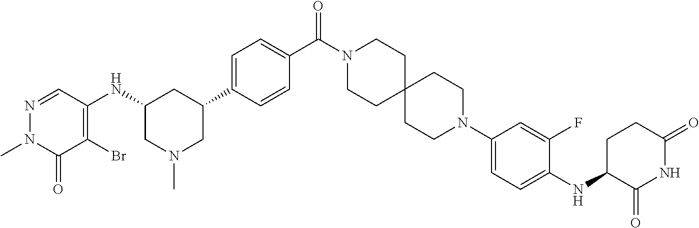 [SMILES: CN1C[C@H](Nc2cnn(C)c(=O)c2Br)C[C@H](c2ccc(C(=O)N3CCC4(CC3)CCN(c3ccc(N[C@H]5CCC(=O)NC5=O)c(F)c3)CC4)cc2)C1] |
| 373a | 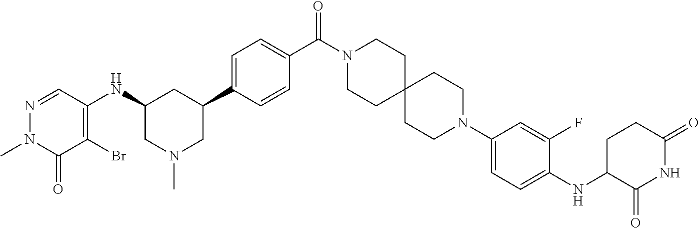 [SMILES: CN1C[C@@H](Nc2cnn(C)c(=O)c2Br)C[C@@H](c2ccc(C(=O)N3CCC4(CC3)CCN(c3ccc(NC5CCC(=O)NC5=O)c(F)c3)CC4)cc2)C1] |
| 373b | 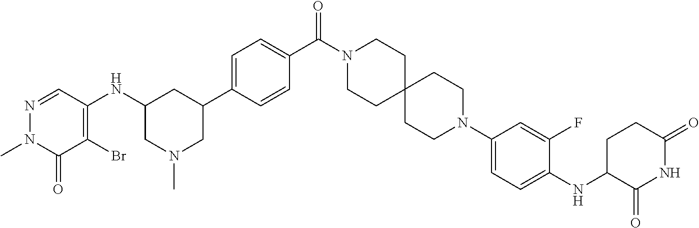 [SMILES: CN1CC(Nc2cnn(C)c(=O)c2Br)CC(c2ccc(C(=O)N3CCC4(CC3)CCN(c3ccc(NC5CCC(=O)NC5=O)c(F)c3)CC4)cc2)C1] |
| 374 | 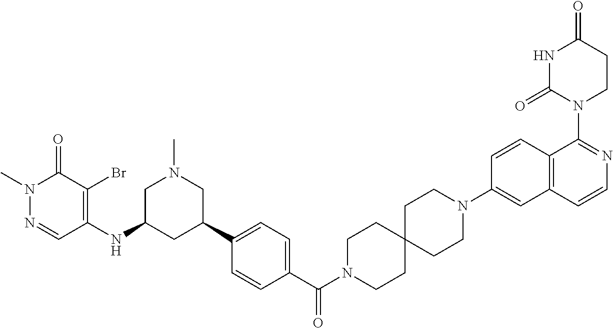 [SMILES: CN1C[C@H](Nc2cnn(C)c(=O)c2Br)C[C@H](c2ccc(C(=O)N3CCC4(CC3)CCN(c3ccc5c(N6CCC(=O)NC6=O)nccc5c3)CC4)cc2)C1] |
| 374a | 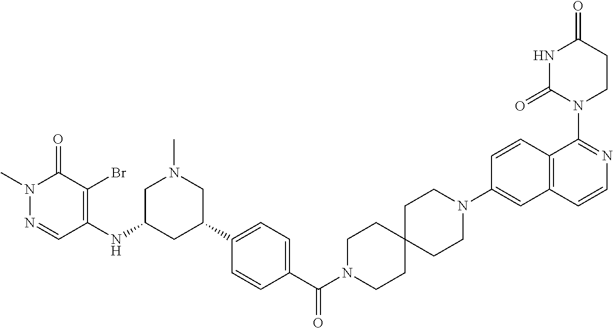 [SMILES: CN1C[C@@H](Nc2cnn(C)c(=O)c2Br)C[C@@H](c2ccc(C(=O)N3CCC4(CC3)CCN(c3ccc5c(N6CCC(=O)NC6=O)nccc5c3)CC4)cc2)C1] |
| 374b | 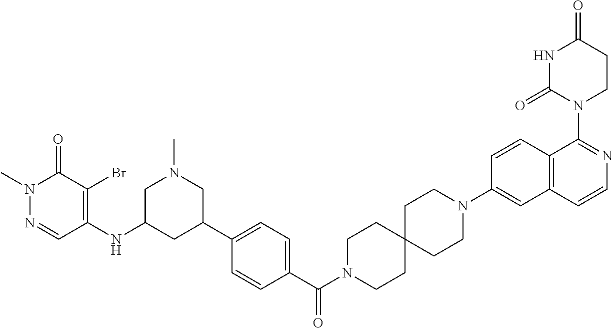 [SMILES: CN1CC(Nc2cnn(C)c(=O)c2Br)CC(c2ccc(C(=O)N3CCC4(CC3)CCN(c3ccc5c(N6CCC(=O)NC6=O)nccc5c3)CC4)cc2)C1] |
| 375 | 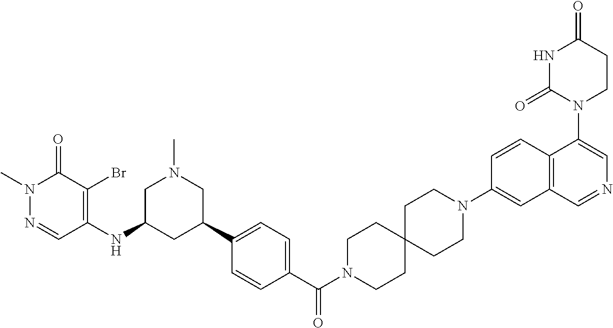 [SMILES: CN1C[C@H](Nc2cnn(C)c(=O)c2Br)C[C@H](c2ccc(C(=O)N3CCC4(CC3)CCN(c3ccc5c(N6CCC(=O)NC6=O)cncc5c3)CC4)cc2)C1] |
| 375a | 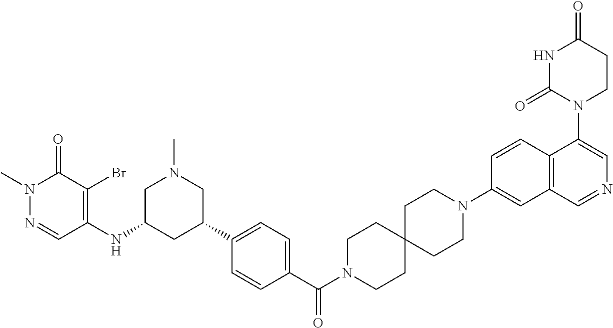 [SMILES: CN1C[C@@H](Nc2cnn(C)c(=O)c2Br)C[C@@H](c2ccc(C(=O)N3CCC4(CC3)CCN(c3ccc5c(N6CCC(=O)NC6=O)cncc5c3)CC4)cc2)C1] |
| 375b | 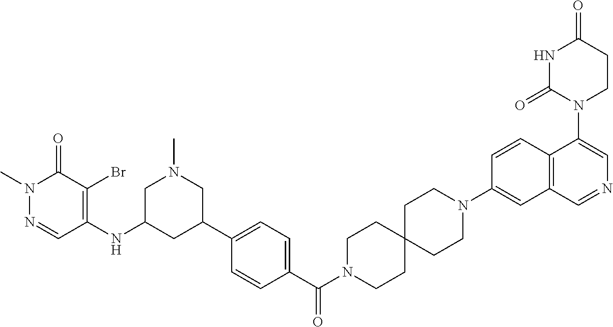 [SMILES: CN1CC(Nc2cnn(C)c(=O)c2Br)CC(c2ccc(C(=O)N3CCC4(CC3)CCN(c3ccc5c(N6CCC(=O)NC6=O)cncc5c3)CC4)cc2)C1] |
| 376 | 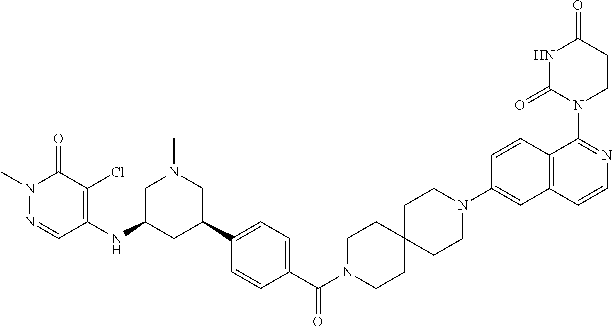 [SMILES: CN1C[C@H](Nc2cnn(C)c(=O)c2Cl)C[C@H](c2ccc(C(=O)N3CCC4(CC3)CCN(c3ccc5c(N6CCC(=O)NC6=O)nccc5c3)CC4)cc2)C1] |
| 376a | 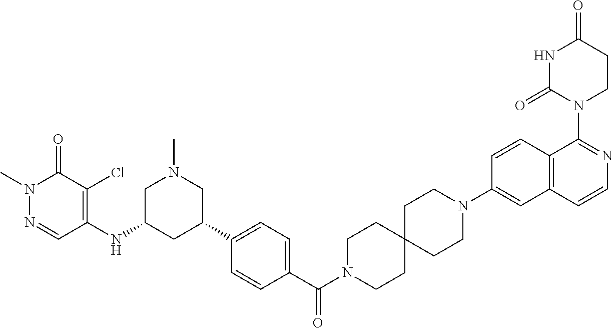 [SMILES: CN1C[C@@H](Nc2cnn(C)c(=O)c2Cl)C[C@@H](c2ccc(C(=O)N3CCC4(CC3)CCN(c3ccc5c(N6CCC(=O)NC6=O)nccc5c3)CC4)cc2)C1] |
| 376b | 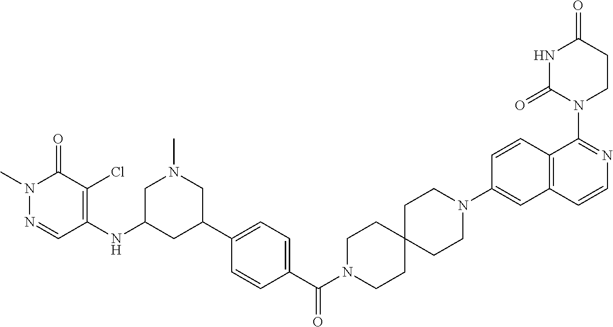 [SMILES: CN1CC(Nc2cnn(C)c(=O)c2Cl)CC(c2ccc(C(=O)N3CCC4(CC3)CCN(c3ccc5c(N6CCC(=O)NC6=O)nccc5c3)CC4)cc2)C1] |
| 377 | 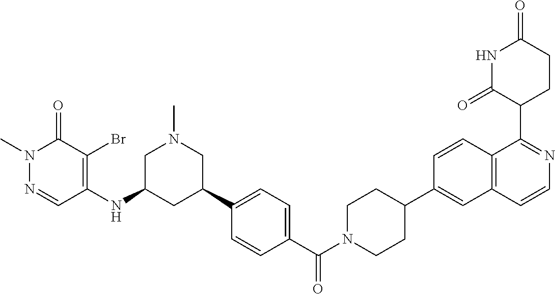 [SMILES: CN1C[C@H](Nc2cnn(C)c(=O)c2Br)C[C@H](c2ccc(C(=O)N3CCC(c4ccc5c(C6CCC(=O)NC6=O)nccc5c4)CC3)cc2)C1] |
| 377a | 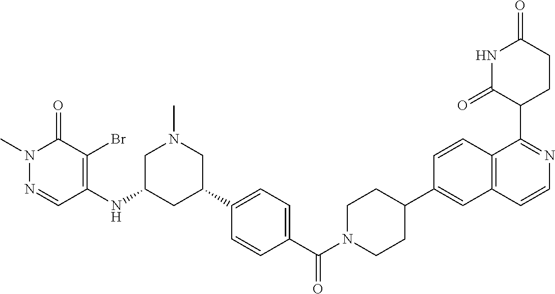 [SMILES: CN1C[C@@H](Nc2cnn(C)c(=O)c2Br)C[C@@H](c2ccc(C(=O)N3CCC(c4ccc5c(C6CCC(=O)NC6=O)nccc5c4)CC3)cc2)C1] |
| 377b | 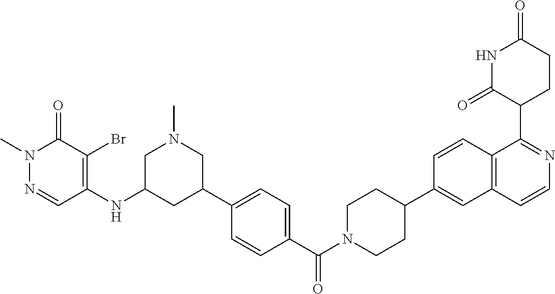 [SMILES: CN1CC(Nc2cnn(C)c(=O)c2Br)CC(c2ccc(C(=O)N3CCC(c4ccc5c(C6CCC(=O)NC6=O)nccc5c4)CC3)cc2)C1] |
| 378 | 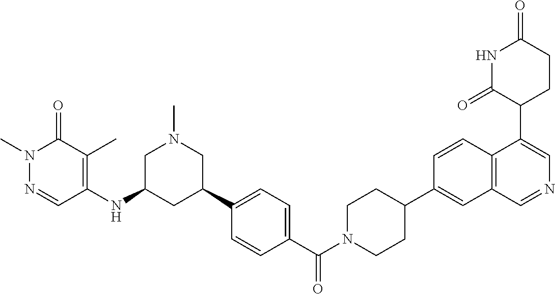 [SMILES: Cc1c(N[C@@H]2C[C@H](c3ccc(C(=O)N4CCC(c5ccc6c(C7CCC(=O)NC7=O)cncc6c5)CC4)cc3)CN(C)C2)cnn(C)c1=O] |
| 378a | 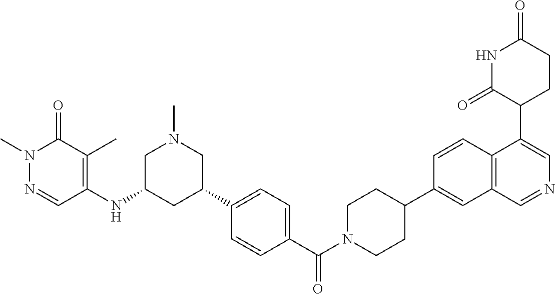 [SMILES: Cc1c(N[C@H]2C[C@@H](c3ccc(C(=O)N4CCC(c5ccc6c(C7CCC(=O)NC7=O)cncc6c5)CC4)cc3)CN(C)C2)cnn(C)c1=O] |
| 378b | 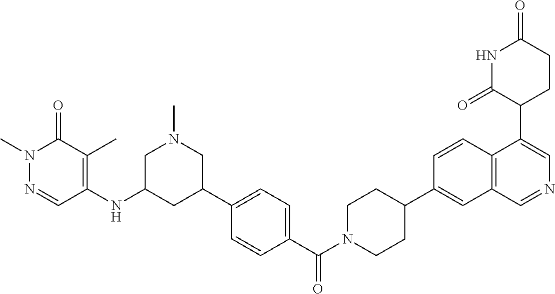 [SMILES: Cc1c(NC2CC(c3ccc(C(=O)N4CCC(c5ccc6c(C7CCC(=O)NC7=O)cncc6c5)CC4)cc3)CN(C)C2)cnn(C)c1=O] |
| 379 | 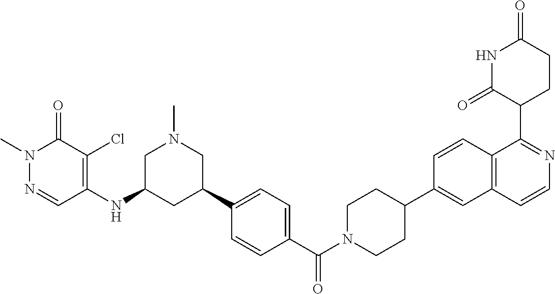 [SMILES: CN1C[C@H](Nc2cnn(C)c(=O)c2Cl)C[C@H](c2ccc(C(=O)N3CCC(c4ccc5c(C6CCC(=O)NC6=O)nccc5c4)CC3)cc2)C1] |
| 379a | 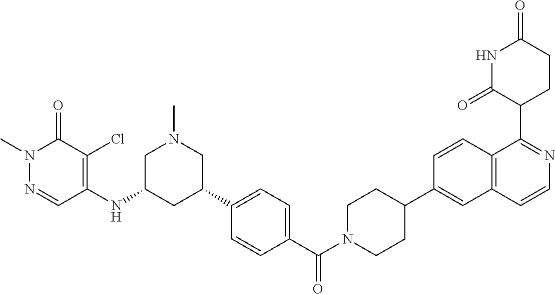 [SMILES: CN1C[C@@H](Nc2cnn(C)c(=O)c2Cl)C[C@@H](c2ccc(C(=O)N3CCC(c4ccc5c(C6CCC(=O)NC6=O)nccc5c4)CC3)cc2)C1] |
| 379b | 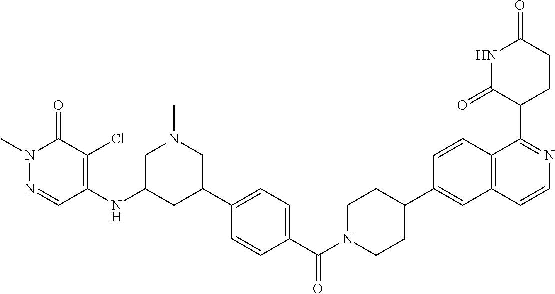 [SMILES: CN1CC(Nc2cnn(C)c(=O)c2Cl)CC(c2ccc(C(=O)N3CCC(c4ccc5c(C6CCC(=O)NC6=O)nccc5c4)CC3)cc2)C1] |
| 380 | 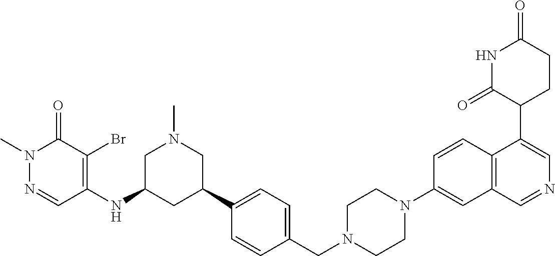 [SMILES: CN1C[C@H](Nc2cnn(C)c(=O)c2Br)C[C@H](c2ccc(CN3CCN(c4ccc5c(C6CCC(=O)NC6=O)cncc5c4)CC3)cc2)C1] |
| 380a | 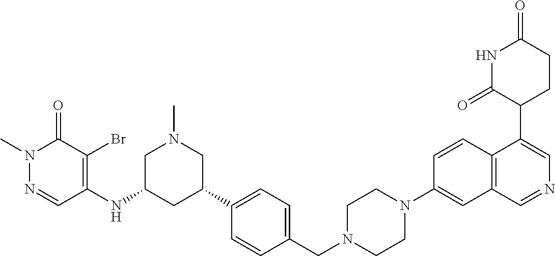 [SMILES: CN1C[C@@H](Nc2cnn(C)c(=O)c2Br)C[C@@H](c2ccc(CN3CCN(c4ccc5c(C6CCC(=O)NC6=O)cncc5c4)CC3)cc2)C1] |
| 380b | 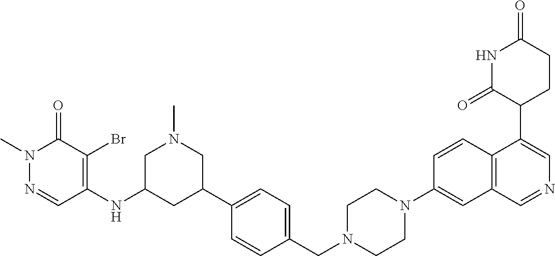 [SMILES: CN1CC(Nc2cnn(C)c(=O)c2Br)CC(c2ccc(CN3CCN(c4ccc5c(C6CCC(=O)NC6=O)cncc5c4)CC3)cc2)C1] |
| 381 | 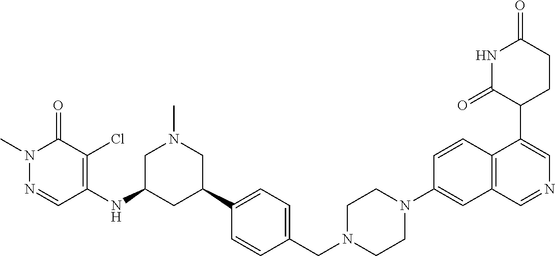 [SMILES: CN1C[C@H](Nc2cnn(C)c(=O)c2Cl)C[C@H](c2ccc(CN3CCN(c4ccc5c(C6CCC(=O)NC6=O)cncc5c4)CC3)cc2)C1] |
| 381a | 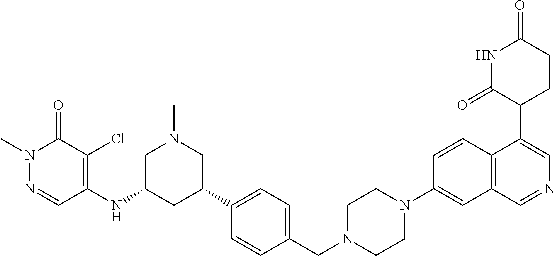 [SMILES: CN1C[C@@H](Nc2cnn(C)c(=O)c2Cl)C[C@@H](c2ccc(CN3CCN(c4ccc5c(C6CCC(=O)NC6=O)cncc5c4)CC3)cc2)C1] |
| 381b | 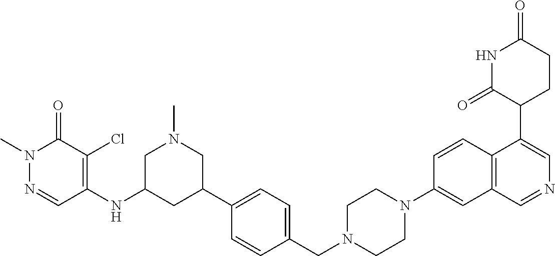 [SMILES: CN1CC(Nc2cnn(C)c(=O)c2Cl)CC(c2ccc(CN3CCN(c4ccc5c(C6CCC(=O)NC6=O)cncc5c4)CC3)cc2)C1] |
| 382 | 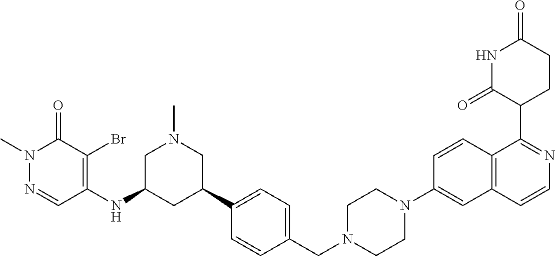 [SMILES: CN1C[C@H](Nc2cnn(C)c(=O)c2Br)C[C@H](c2ccc(CN3CCN(c4ccc5c(C6CCC(=O)NC6=O)nccc5c4)CC3)cc2)C1] |
| 382a | 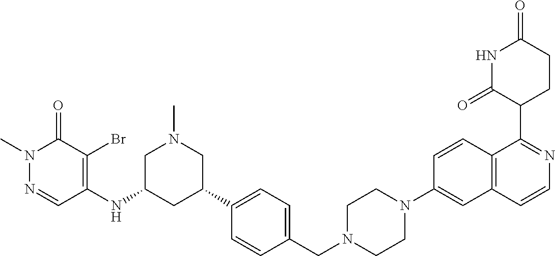 [SMILES: CN1C[C@@H](Nc2cnn(C)c(=O)c2Br)C[C@@H](c2ccc(CN3CCN(c4ccc5c(C6CCC(=O)NC6=O)nccc5c4)CC3)cc2)C1] |
| 382b | 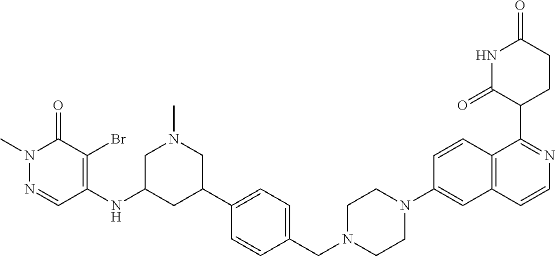 [SMILES: CN1CC(Nc2cnn(C)c(=O)c2Br)CC(c2ccc(CN3CCN(c4ccc5c(C6CCC(=O)NC6=O)nccc5c4)CC3)cc2)C1] |
| 383 | 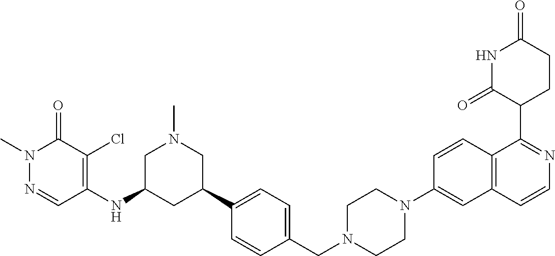 [SMILES: CN1C[C@H](Nc2cnn(C)c(=O)c2Cl)C[C@H](c2ccc(CN3CCN(c4ccc5c(C6CCC(=O)NC6=O)nccc5c4)CC3)cc2)C1] |
| 383a | 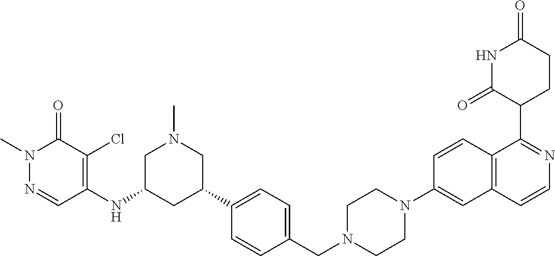 [SMILES: CN1C[C@@H](Nc2cnn(C)c(=O)c2Cl)C[C@@H](c2ccc(CN3CCN(c4ccc5c(C6CCC(=O)NC6=O)nccc5c4)CC3)cc2)C1] |
| 383b | 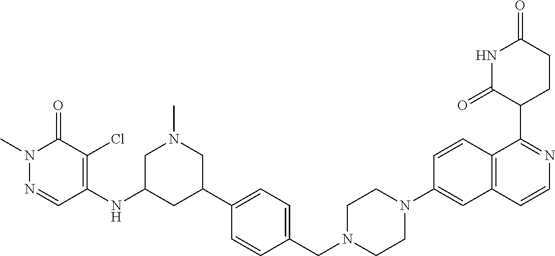 [SMILES: CN1CC(Nc2cnn(C)c(=O)c2Cl)CC(c2ccc(CN3CCN(c4ccc5c(C6CCC(=O)NC6=O)nccc5c4)CC3)cc2)C1] |
| 384 | 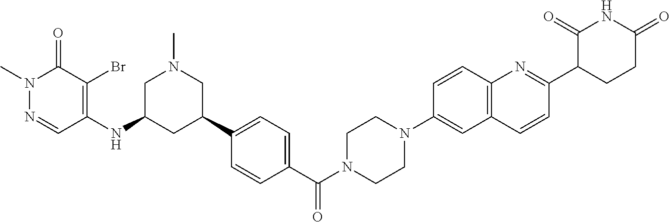 [SMILES: CN1C[C@H](Nc2cnn(C)c(=O)c2Br)C[C@H](c2ccc(C(=O)N3CCN(c4ccc5nc(C6CCC(=O)NC6=O)ccc5c4)CC3)cc2)C1] |
| 384a | 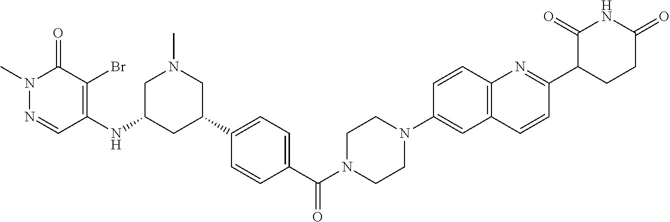 [SMILES: CN1C[C@@H](Nc2cnn(C)c(=O)c2Br)C[C@@H](c2ccc(C(=O)N3CCN(c4ccc5nc(C6CCC(=O)NC6=O)ccc5c4)CC3)cc2)C1] |
| 384b | 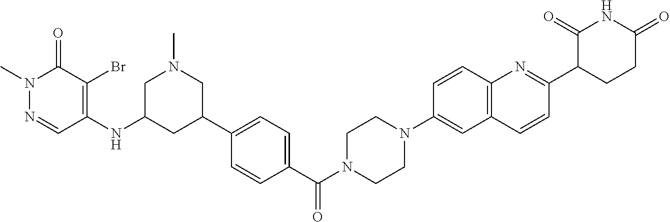 [SMILES: CN1CC(Nc2cnn(C)c(=O)c2Br)CC(c2ccc(C(=O)N3CCN(c4ccc5nc(C6CCC(=O)NC6=O)ccc5c4)CC3)cc2)C1] |
| 385 | 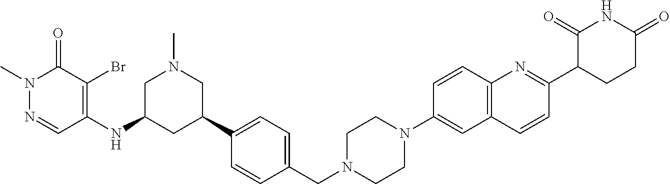 [SMILES: CN1C[C@H](Nc2cnn(C)c(=O)c2Br)C[C@H](c2ccc(CN3CCN(c4ccc5nc(C6CCC(=O)NC6=O)ccc5c4)CC3)cc2)C1] |
| 385a | 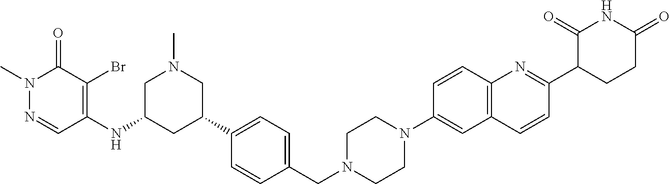 [SMILES: CN1C[C@@H](Nc2cnn(C)c(=O)c2Br)C[C@@H](c2ccc(CN3CCN(c4ccc5nc(C6CCC(=O)NC6=O)ccc5c4)CC3)cc2)C1] |
| 385b | 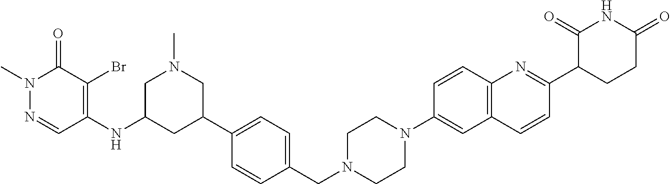 [SMILES: CN1CC(Nc2cnn(C)c(=O)c2Br)CC(c2ccc(CN3CCN(c4ccc5nc(C6CCC(=O)NC6=O)ccc5c4)CC3)cc2)C1] |
| 386 | 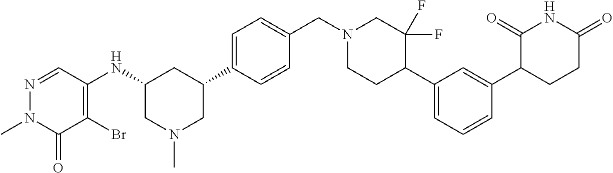 [SMILES: CN1C[C@H](Nc2cnn(C)c(=O)c2Br)C[C@H](c2ccc(CN3CCC(c4cccc(C5CCC(=O)NC5=O)c4)C(F)(F)C3)cc2)C1] |
| 386a | 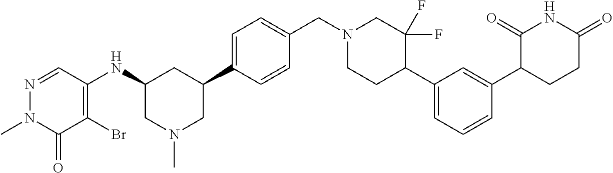 [SMILES: CN1C[C@@H](Nc2cnn(C)c(=O)c2Br)C[C@@H](c2ccc(CN3CCC(c4cccc(C5CCC(=O)NC5=O)c4)C(F)(F)C3)cc2)C1] |
| 386b | 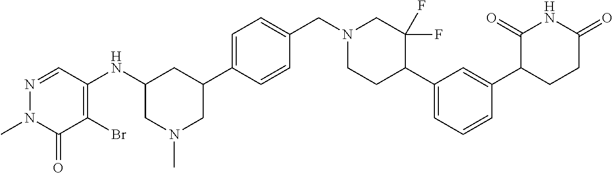 [SMILES: CN1CC(Nc2cnn(C)c(=O)c2Br)CC(c2ccc(CN3CCC(c4cccc(C5CCC(=O)NC5=O)c4)C(F)(F)C3)cc2)C1] |
| 387 | 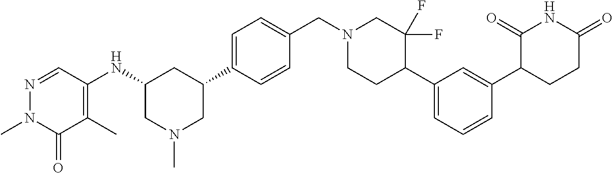 [SMILES: Cc1c(N[C@@H]2C[C@H](c3ccc(CN4CCC(c5cccc(C6CCC(=O)NC6=O)c5)C(F)(F)C4)cc3)CN(C)C2)cnn(C)c1=O] |
| 387a | 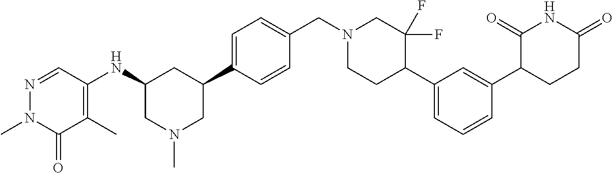 [SMILES: Cc1c(N[C@H]2C[C@@H](c3ccc(CN4CCC(c5cccc(C6CCC(=O)NC6=O)c5)C(F)(F)C4)cc3)CN(C)C2)cnn(C)c1=O] |
| 387b | 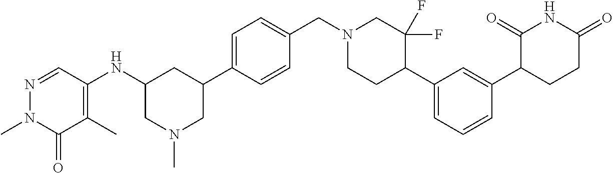 [SMILES: Cc1c(NC2CC(c3ccc(CN4CCC(c5cccc(C6CCC(=O)NC6=O)c5)C(F)(F)C4)cc3)CN(C)C2)cnn(C)c1=O] |
| 388 | 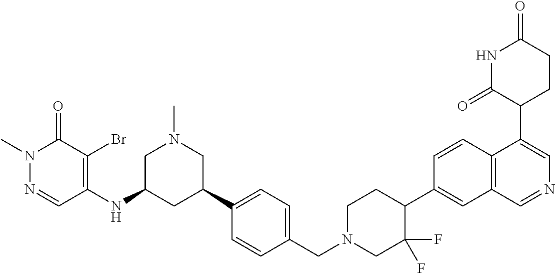 [SMILES: CN1C[C@H](Nc2cnn(C)c(=O)c2Br)C[C@H](c2ccc(CN3CCC(c4ccc5c(C6CCC(=O)NC6=O)cncc5c4)C(F)(F)C3)cc2)C1] |
| 388a | 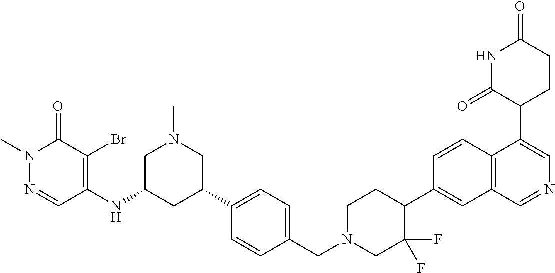 [SMILES: CN1C[C@@H](Nc2cnn(C)c(=O)c2Br)C[C@@H](c2ccc(CN3CCC(c4ccc5c(C6CCC(=O)NC6=O)cncc5c4)C(F)(F)C3)cc2)C1] |
| 388b | 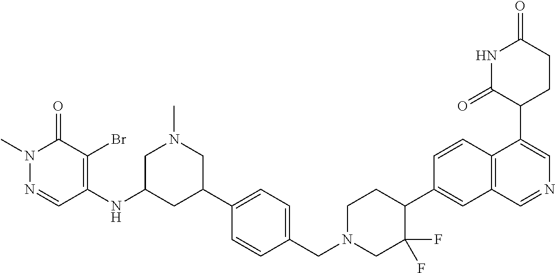 [SMILES: CN1CC(Nc2cnn(C)c(=O)c2Br)CC(c2ccc(CN3CCC(c4ccc5c(C6CCC(=O)NC6=O)cncc5c4)C(F)(F)C3)cc2)C1] |
| 389 | 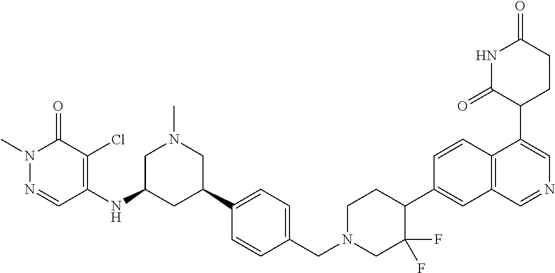 [SMILES: CN1C[C@H](Nc2cnn(C)c(=O)c2Cl)C[C@H](c2ccc(CN3CCC(c4ccc5c(C6CCC(=O)NC6=O)cncc5c4)C(F)(F)C3)cc2)C1] |
| 389a | 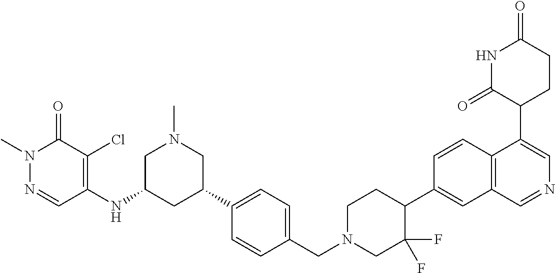 [SMILES: CN1C[C@@H](Nc2cnn(C)c(=O)c2Cl)C[C@@H](c2ccc(CN3CCC(c4ccc5c(C6CCC(=O)NC6=O)cncc5c4)C(F)(F)C3)cc2)C1] |
| 389b | 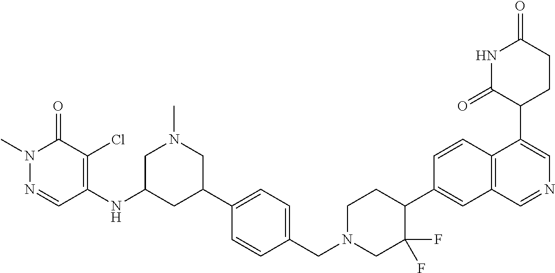 [SMILES: CN1CC(Nc2cnn(C)c(=O)c2Cl)CC(c2ccc(CN3CCC(c4ccc5c(C6CCC(=O)NC6=O)cncc5c4)C(F)(F)C3)cc2)C1] |
| 390 | 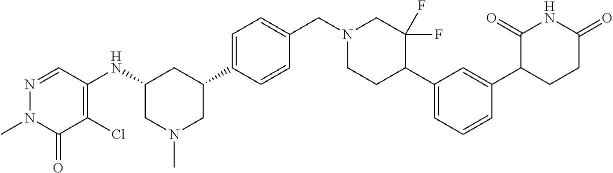 [SMILES: CN1C[C@H](Nc2cnn(C)c(=O)c2Cl)C[C@H](c2ccc(CN3CCC(c4cccc(C5CCC(=O)NC5=O)c4)C(F)(F)C3)cc2)C1] |
| 390a | 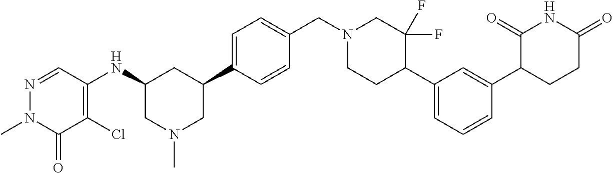 [SMILES: CN1C[C@@H](Nc2cnn(C)c(=O)c2Cl)C[C@@H](c2ccc(CN3CCC(c4cccc(C5CCC(=O)NC5=O)c4)C(F)(F)C3)cc2)C1] |
| 390b | 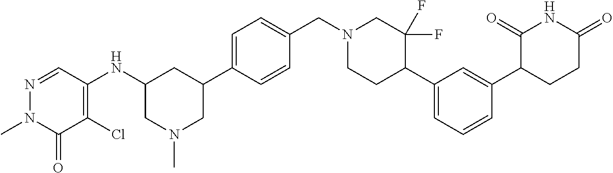 [SMILES: CN1CC(Nc2cnn(C)c(=O)c2Cl)CC(c2ccc(CN3CCC(c4cccc(C5CCC(=O)NC5=O)c4)C(F)(F)C3)cc2)C1] |
| 391 | 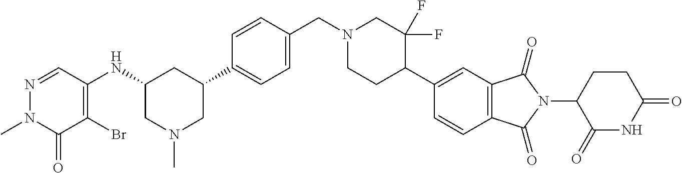 [SMILES: CN1C[C@H](Nc2cnn(C)c(=O)c2Br)C[C@H](c2ccc(CN3CCC(c4ccc5c(c4)C(=O)N(C4CCC(=O)NC4=O)C5=O)C(F)(F)C3)cc2)C1] |
| 391a | 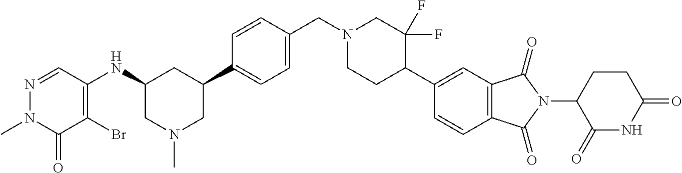 [SMILES: CN1C[C@@H](Nc2cnn(C)c(=O)c2Br)C[C@@H](c2ccc(CN3CCC(c4ccc5c(c4)C(=O)N(C4CCC(=O)NC4=O)C5=O)C(F)(F)C3)cc2)C1] |
| 391b | 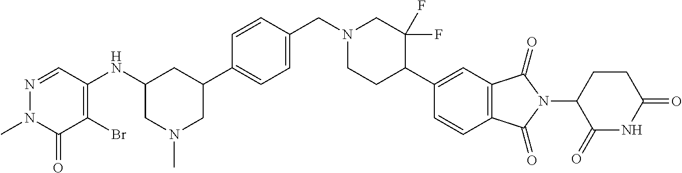 [SMILES: CN1CC(Nc2cnn(C)c(=O)c2Br)CC(c2ccc(CN3CCC(c4ccc5c(c4)C(=O)N(C4CCC(=O)NC4=O)C5=O)C(F)(F)C3)cc2)C1] |
| 392 | 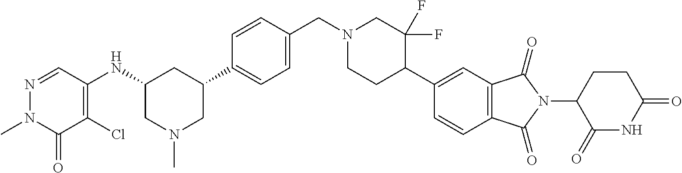 [SMILES: CN1C[C@H](Nc2cnn(C)c(=O)c2Cl)C[C@H](c2ccc(CN3CCC(c4ccc5c(c4)C(=O)N(C4CCC(=O)NC4=O)C5=O)C(F)(F)C3)cc2)C1] |
| 392a | 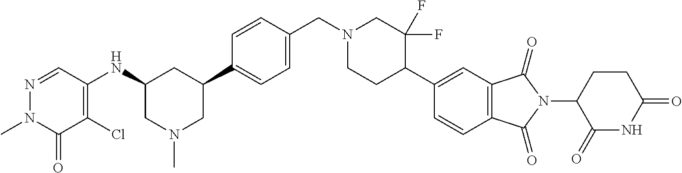 [SMILES: CN1C[C@@H](Nc2cnn(C)c(=O)c2Cl)C[C@@H](c2ccc(CN3CCC(c4ccc5c(c4)C(=O)N(C4CCC(=O)NC4=O)C5=O)C(F)(F)C3)cc2)C1] |
| 392b | 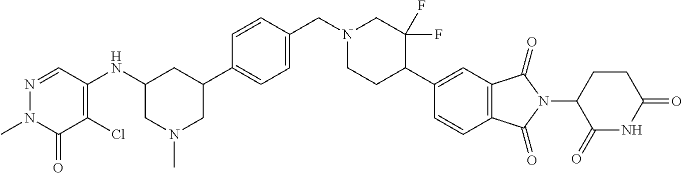 [SMILES: CN1CC(Nc2cnn(C)c(=O)c2Cl)CC(c2ccc(CN3CCC(c4ccc5c(c4)C(=O)N(C4CCC(=O)NC4=O)C5=O)C(F)(F)C3)cc2)C1] |
| 393 | 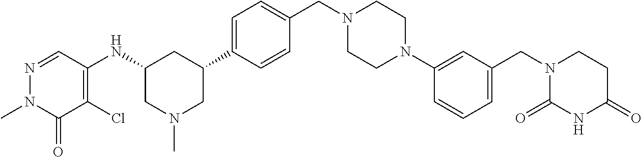 [SMILES: CN1C[C@H](Nc2cnn(C)c(=O)c2Cl)C[C@H](c2ccc(CN3CCN(c4cccc(CN5CCC(=O)NC5=O)c4)CC3)cc2)C1] |
| 393a | 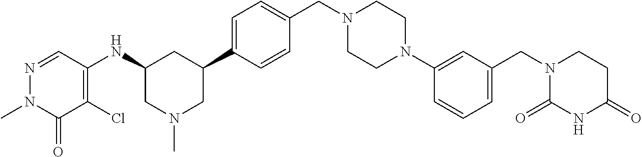 [SMILES: CN1C[C@@H](Nc2cnn(C)c(=O)c2Cl)C[C@@H](c2ccc(CN3CCN(c4cccc(CN5CCC(=O)NC5=O)c4)CC3)cc2)C1] |
| 393b | 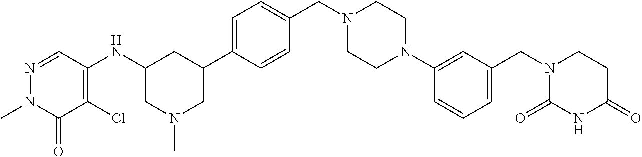 [SMILES: CN1CC(Nc2cnn(C)c(=O)c2Cl)CC(c2ccc(CN3CCN(c4cccc(CN5CCC(=O)NC5=O)c4)CC3)cc2)C1] |
| 394 | 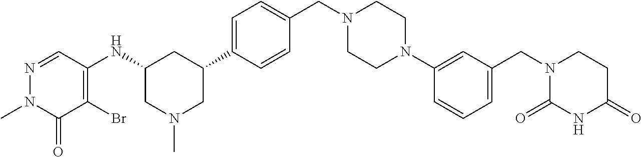 [SMILES: CN1C[C@H](Nc2cnn(C)c(=O)c2Br)C[C@H](c2ccc(CN3CCN(c4cccc(CN5CCC(=O)NC5=O)c4)CC3)cc2)C1] |
| 394a | 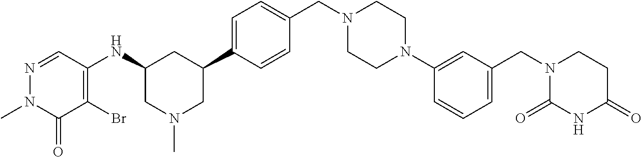 [SMILES: CN1C[C@@H](Nc2cnn(C)c(=O)c2Br)C[C@@H](c2ccc(CN3CCN(c4cccc(CN5CCC(=O)NC5=O)c4)CC3)cc2)C1] |
| 394b | 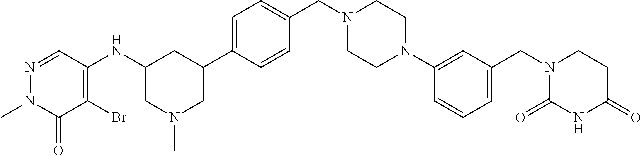 [SMILES: CN1CC(Nc2cnn(C)c(=O)c2Br)CC(c2ccc(CN3CCN(c4cccc(CN5CCC(=O)NC5=O)c4)CC3)cc2)C1] |
| 395 | 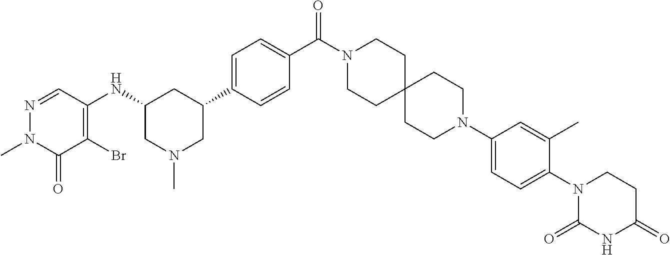 [SMILES: Cc1cc(N2CCC3(CCN(C(=O)c4ccc([C@H]5C[C@@H](Nc6cnn(C)c(=O)c6Br)CN(C)C5)cc4)CC3)CC2)ccc1N1CCC(=O)NC1=O] |
| 395a | 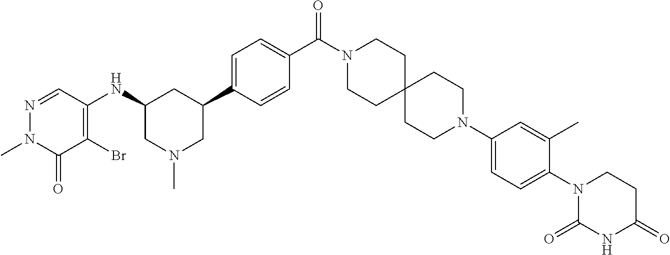 [SMILES: Cc1cc(N2CCC3(CCN(C(=O)c4ccc([C@@H]5C[C@H](Nc6cnn(C)c(=O)c6Br)CN(C)C5)cc4)CC3)CC2)ccc1N1CCC(=O)NC1=O] |
| 395b | 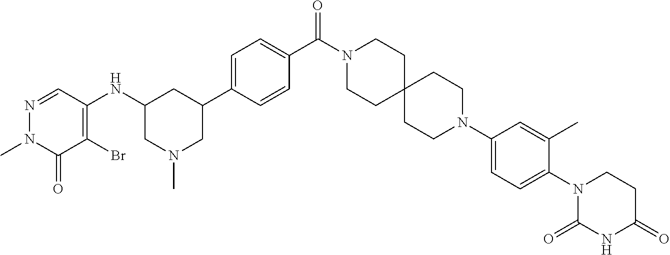 [SMILES: Cc1cc(N2CCC3(CCN(C(=O)c4ccc(C5CC(Nc6cnn(C)c(=O)c6Br)CN(C)C5)cc4)CC3)CC2)ccc1N1CCC(=O)NC1=O] |
| 396 | 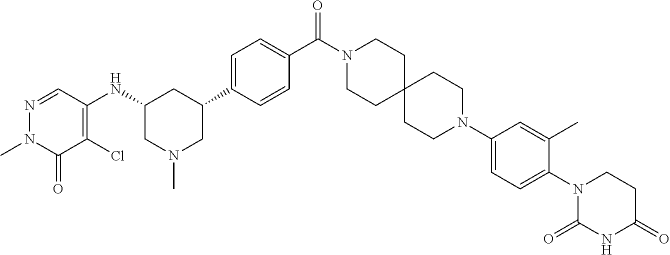 [SMILES: Cc1cc(N2CCC3(CCN(C(=O)c4ccc([C@H]5C[C@@H](Nc6cnn(C)c(=O)c6Cl)CN(C)C5)cc4)CC3)CC2)ccc1N1CCC(=O)NC1=O] |
| 396a | 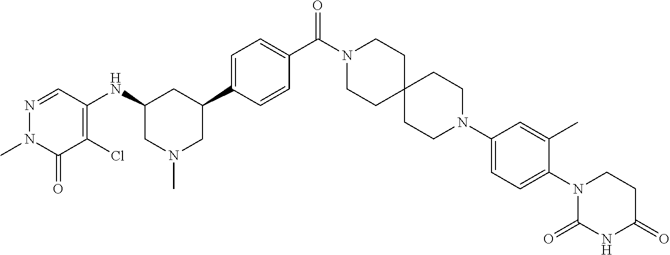 [SMILES: Cc1cc(N2CCC3(CCN(C(=O)c4ccc([C@@H]5C[C@H](Nc6cnn(C)c(=O)c6Cl)CN(C)C5)cc4)CC3)CC2)ccc1N1CCC(=O)NC1=O] |
| 396b | 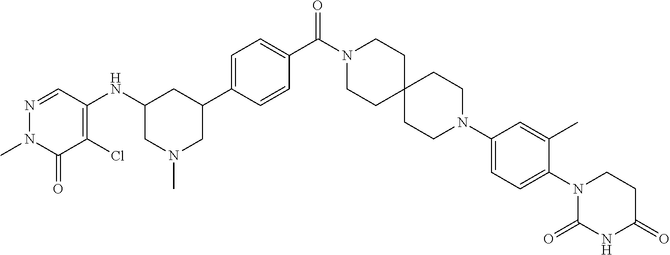 [SMILES: Cc1cc(N2CCC3(CCN(C(=O)c4ccc(C5CC(Nc6cnn(C)c(=O)c6Cl)CN(C)C5)cc4)CC3)CC2)ccc1N1CCC(=O)NC1=O] |
| 397 | 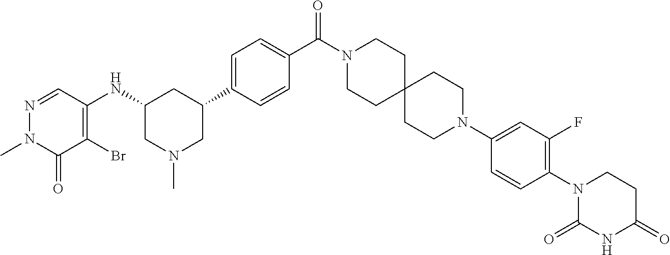 [SMILES: CN1C[C@H](Nc2cnn(C)c(=O)c2Br)C[C@H](c2ccc(C(=O)N3CCC4(CC3)CCN(c3ccc(N5CCC(=O)NC5=O)c(F)c3)CC4)cc2)C1] |
| 397a | 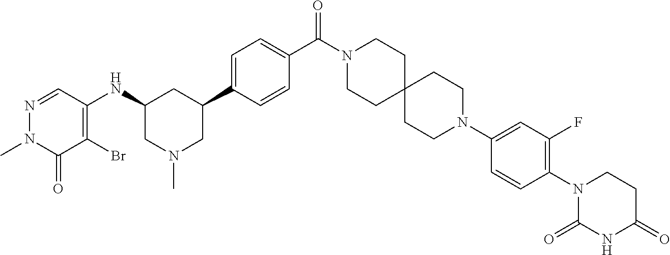 [SMILES: CN1C[C@@H](Nc2cnn(C)c(=O)c2Br)C[C@@H](c2ccc(C(=O)N3CCC4(CC3)CCN(c3ccc(N5CCC(=O)NC5=O)c(F)c3)CC4)cc2)C1] |
| 397b | 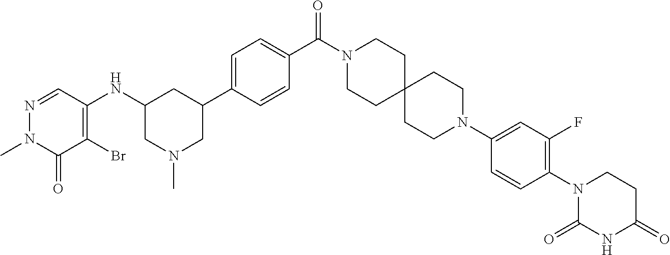 [SMILES: CN1CC(Nc2cnn(C)c(=O)c2Br)CC(c2ccc(C(=O)N3CCC4(CC3)CCN(c3ccc(N5CCC(=O)NC5=O)c(F)c3)CC4)cc2)C1] |
| 398 | 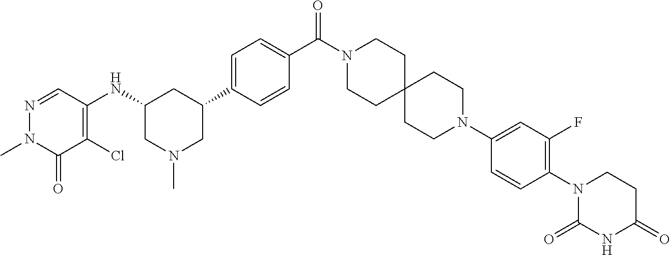 [SMILES: CN1C[C@H](Nc2cnn(C)c(=O)c2Cl)C[C@H](c2ccc(C(=O)N3CCC4(CC3)CCN(c3ccc(N5CCC(=O)NC5=O)c(F)c3)CC4)cc2)C1] |
| 398a | 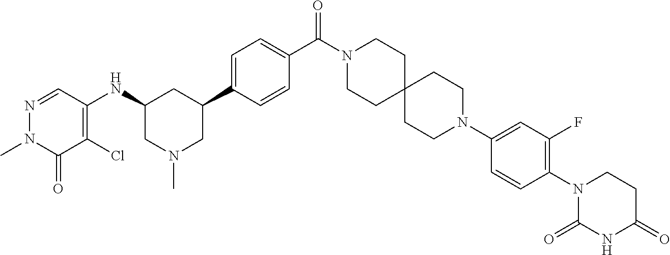 [SMILES: CN1C[C@@H](Nc2cnn(C)c(=O)c2Cl)C[C@@H](c2ccc(C(=O)N3CCC4(CC3)CCN(c3ccc(N5CCC(=O)NC5=O)c(F)c3)CC4)cc2)C1] |
| 398b | 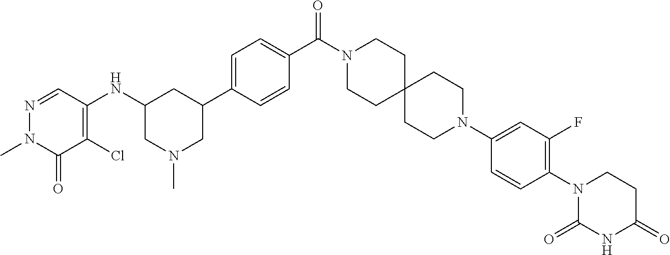 [SMILES: CN1CC(Nc2cnn(C)c(=O)c2Cl)CC(c2ccc(C(=O)N3CCC4(CC3)CCN(c3ccc(N5CCC(=O)NC5=O)c(F)c3)CC4)cc2)C1] |
| 399 | 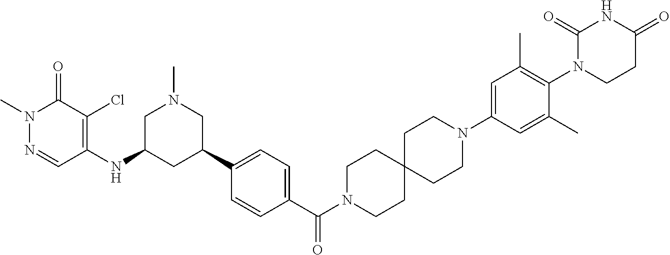 [SMILES: Cc1cc(N2CCC3(CCN(C(=O)c4ccc([C@H]5C[C@@H](Nc6cnn(C)c(=O)c6Cl)CN(C)C5)cc4)CC3)CC2)cc(C)c1N1CCC(=O)NC1=O] |
| 399a | 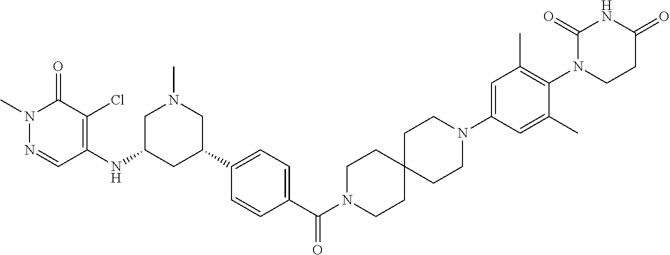 [SMILES: Cc1cc(N2CCC3(CCN(C(=O)c4ccc([C@@H]5C[C@H](Nc6cnn(C)c(=O)c6Cl)CN(C)C5)cc4)CC3)CC2)cc(C)c1N1CCC(=O)NC1=O] |
| 399b | 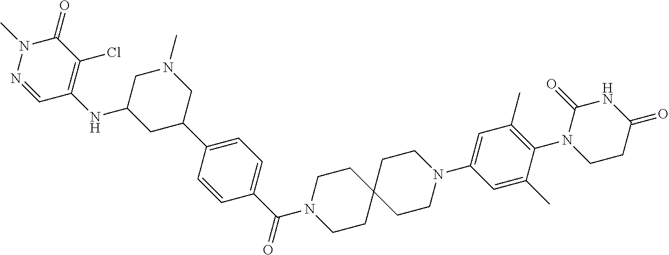 [SMILES: Cc1cc(N2CCC3(CCN(C(=O)c4ccc(C5CC(Nc6cnn(C)c(=O)c6Cl)CN(C)C5)cc4)CC3)CC2)cc(C)c1N1CCC(=O)NC1=O] |
| 400 | 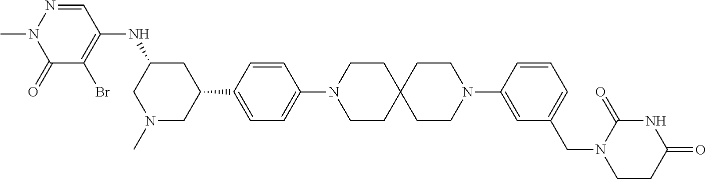 [SMILES: CN1C[C@H](Nc2cnn(C)c(=O)c2Br)C[C@H](c2ccc(N3CCC4(CC3)CCN(c3cccc(CN5CCC(=O)NC5=O)c3)CC4)cc2)C1] |
| 400a | 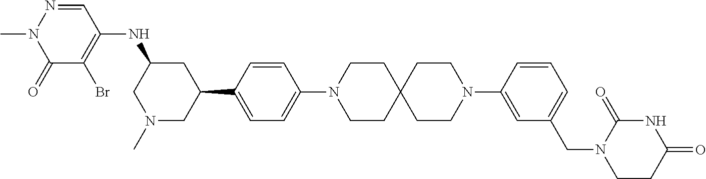 [SMILES: CN1C[C@@H](Nc2cnn(C)c(=O)c2Br)C[C@@H](c2ccc(N3CCC4(CC3)CCN(c3cccc(CN5CCC(=O)NC5=O)c3)CC4)cc2)C1] |
| 400b | 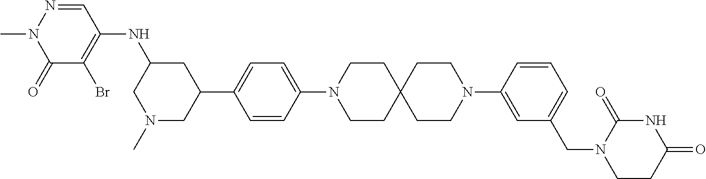 [SMILES: CN1CC(Nc2cnn(C)c(=O)c2Br)CC(c2ccc(N3CCC4(CC3)CCN(c3cccc(CN5CCC(=O)NC5=O)c3)CC4)cc2)C1] |
| 401 | 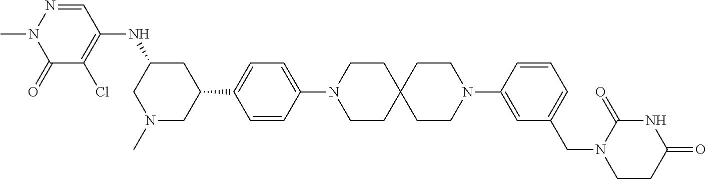 [SMILES: CN1C[C@H](Nc2cnn(C)c(=O)c2Cl)C[C@H](c2ccc(N3CCC4(CC3)CCN(c3cccc(CN5CCC(=O)NC5=O)c3)CC4)cc2)C1] |
| 401a | 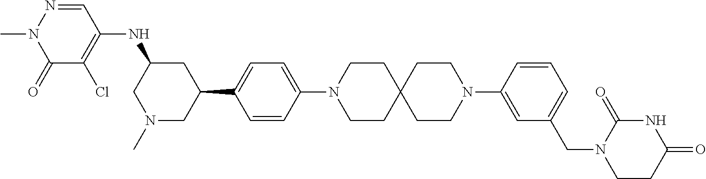 [SMILES: CN1C[C@@H](Nc2cnn(C)c(=O)c2Cl)C[C@@H](c2ccc(N3CCC4(CC3)CCN(c3cccc(CN5CCC(=O)NC5=O)c3)CC4)cc2)C1] |
| 401b | 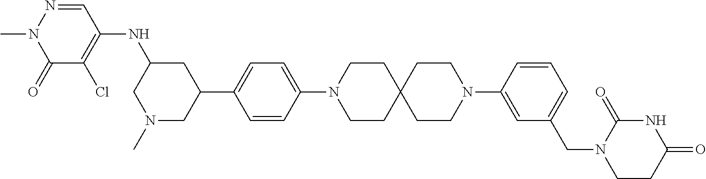 [SMILES: CN1CC(Nc2cnn(C)c(=O)c2Cl)CC(c2ccc(N3CCC4(CC3)CCN(c3cccc(CN5CCC(=O)NC5=O)c3)CC4)cc2)C1] |
| 402 | 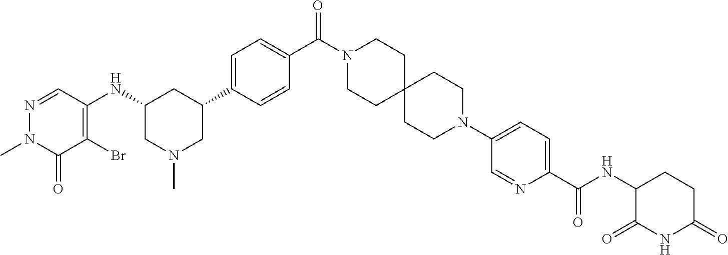 [SMILES: CN1C[C@H](Nc2cnn(C)c(=O)c2Br)C[C@H](c2ccc(C(=O)N3CCC4(CC3)CCN(c3ccc(C(=O)NC5CCC(=O)NC5=O)nc3)CC4)cc2)C1] |
| 402-1 | 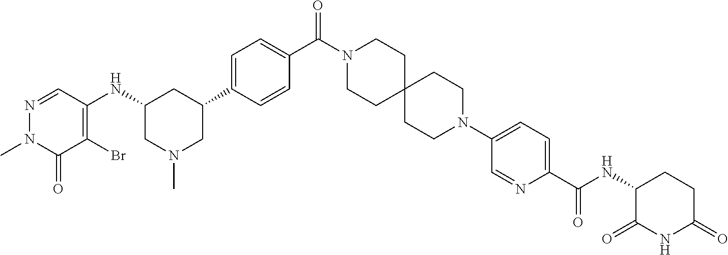 [SMILES: CN1C[C@H](Nc2cnn(C)c(=O)c2Br)C[C@H](c2ccc(C(=O)N3CCC4(CC3)CCN(c3ccc(C(=O)N[C@@H]5CCC(=O)NC5=O)nc3)CC4)cc2)C1] |
| 402-2 | 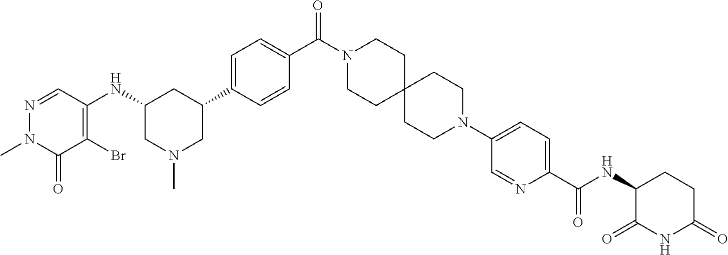 [SMILES: CN1C[C@H](Nc2cnn(C)c(=O)c2Br)C[C@H](c2ccc(C(=O)N3CCC4(CC3)CCN(c3ccc(C(=O)N[C@H]5CCC(=O)NC5=O)nc3)CC4)cc2)C1] |
| 402a | 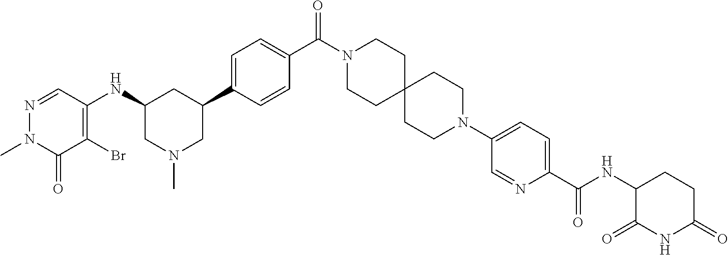 [SMILES: CN1C[C@@H](Nc2cnn(C)c(=O)c2Br)C[C@@H](c2ccc(C(=O)N3CCC4(CC3)CCN(c3ccc(C(=O)NC5CCC(=O)NC5=O)nc3)CC4)cc2)C1] |
| 402b | 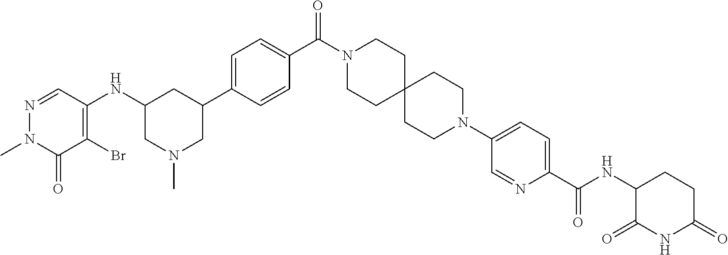 [SMILES: CN1CC(Nc2cnn(C)c(=O)c2Br)CC(c2ccc(C(=O)N3CCC4(CC3)CCN(c3ccc(C(=O)NC5CCC(=O)NC5=O)nc3)CC4)cc2)C1] |
| 403 | 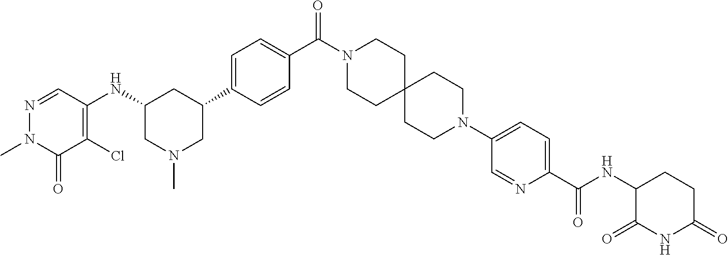 [SMILES: CN1C[C@H](Nc2cnn(C)c(=O)c2Cl)C[C@H](c2ccc(C(=O)N3CCC4(CC3)CCN(c3ccc(C(=O)NC5CCC(=O)NC5=O)nc3)CC4)cc2)C1] |
| 403a | 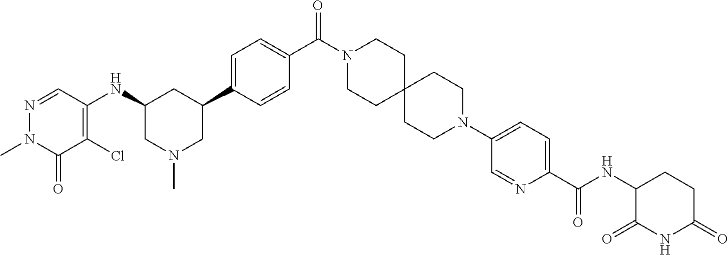 [SMILES: CN1C[C@@H](Nc2cnn(C)c(=O)c2Cl)C[C@@H](c2ccc(C(=O)N3CCC4(CC3)CCN(c3ccc(C(=O)NC5CCC(=O)NC5=O)nc3)CC4)cc2)C1] |
| 403b | 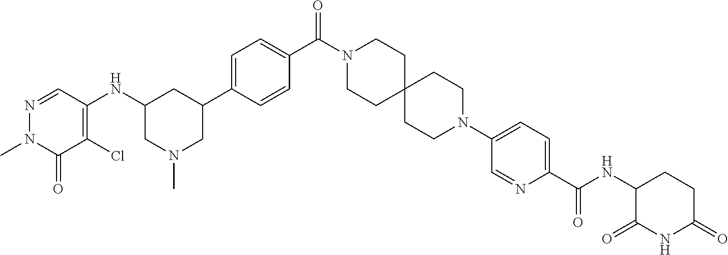 [SMILES: CN1CC(Nc2cnn(C)c(=O)c2Cl)CC(c2ccc(C(=O)N3CCC4(CC3)CCN(c3ccc(C(=O)NC5CCC(=O)NC5=O)nc3)CC4)cc2)C1] |
| 404 | 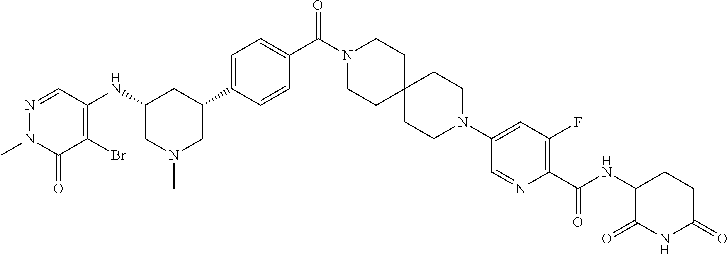 [SMILES: CN1C[C@H](Nc2cnn(C)c(=O)c2Br)C[C@H](c2ccc(C(=O)N3CCC4(CC3)CCN(c3cnc(C(=O)NC5CCC(=O)NC5=O)c(F)c3)CC4)cc2)C1] |
| 404a | 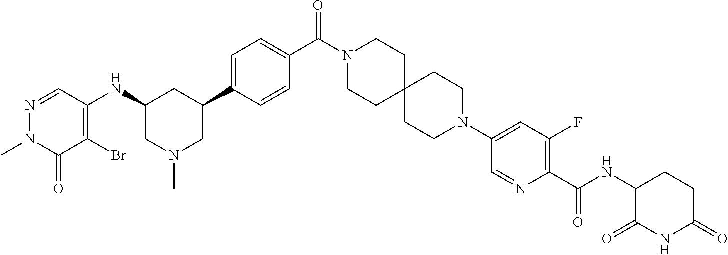 [SMILES: CN1C[C@@H](Nc2cnn(C)c(=O)c2Br)C[C@@H](c2ccc(C(=O)N3CCC4(CC3)CCN(c3cnc(C(=O)NC5CCC(=O)NC5=O)c(F)c3)CC4)cc2)C1] |
| 404b | 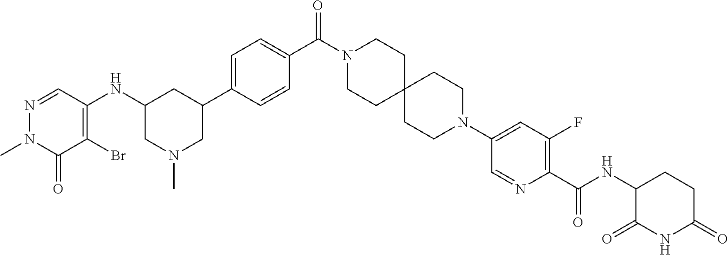 [SMILES: CN1CC(Nc2cnn(C)c(=O)c2Br)CC(c2ccc(C(=O)N3CCC4(CC3)CCN(c3cnc(C(=O)NC5CCC(=O)NC5=O)c(F)c3)CC4)cc2)C1] |
| 405 | 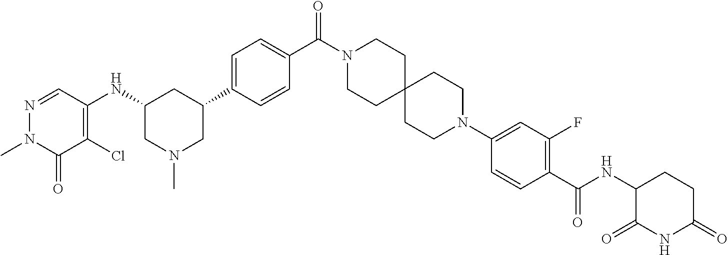 [SMILES: CN1C[C@H](Nc2cnn(C)c(=O)c2Cl)C[C@H](c2ccc(C(=O)N3CCC4(CC3)CCN(c3ccc(C(=O)NC5CCC(=O)NC5=O)c(F)c3)CC4)cc2)C1] |
| 405a | 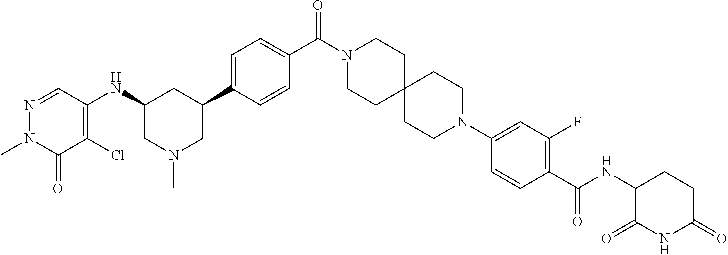 [SMILES: CN1C[C@@H](Nc2cnn(C)c(=O)c2Cl)C[C@@H](c2ccc(C(=O)N3CCC4(CC3)CCN(c3ccc(C(=O)NC5CCC(=O)NC5=O)c(F)c3)CC4)cc2)C1] |
| 405b | 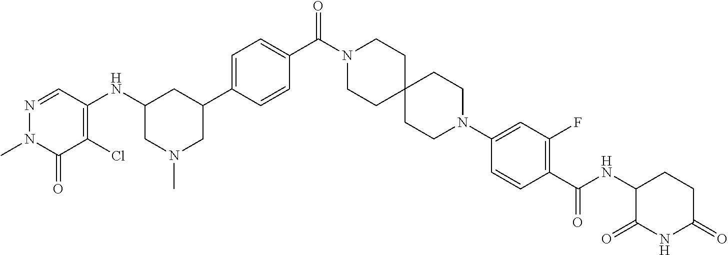 [SMILES: CN1CC(Nc2cnn(C)c(=O)c2Cl)CC(c2ccc(C(=O)N3CCC4(CC3)CCN(c3ccc(C(=O)NC5CCC(=O)NC5=O)c(F)c3)CC4)cc2)C1] |
| 406 | 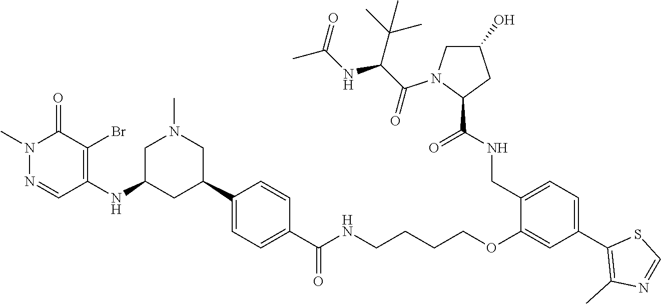 [SMILES: CC(=O)N[C@H](C(=O)N1C[C@H](O)C[C@H]1C(=O)NCc1ccc(-c2scnc2C)cc1OCCCCNC(=O)c1ccc([C@H]2C[C@@H](Nc3cnn(C)c(=O)c3Br)CN(C)C2)cc1)C(C)(C)C] |
| 406a | 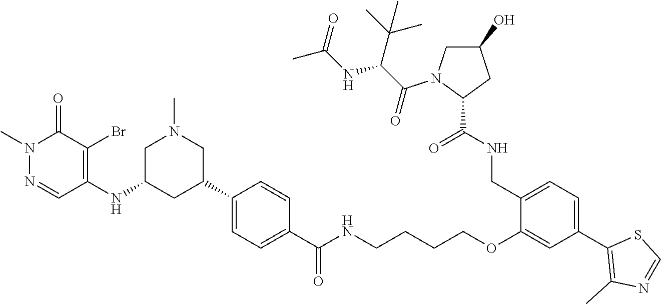 |
| 406b | 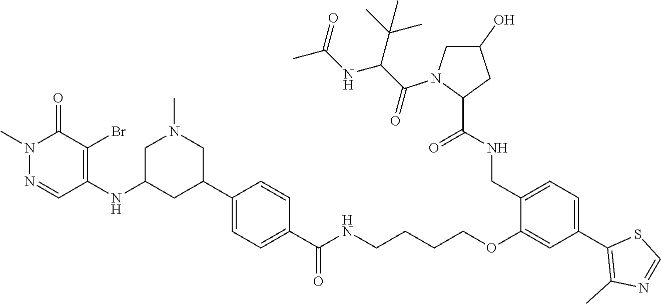 [SMILES: CC(=O)NC(C(=O)N1CC(O)CC1C(=O)NCc1ccc(-c2scnc2C)cc1OCCCCNC(=O)c1ccc(C2CC(Nc3cnn(C)c(=O)c3Br)CN(C)C2)cc1)C(C)(C)C] |
| 407 | 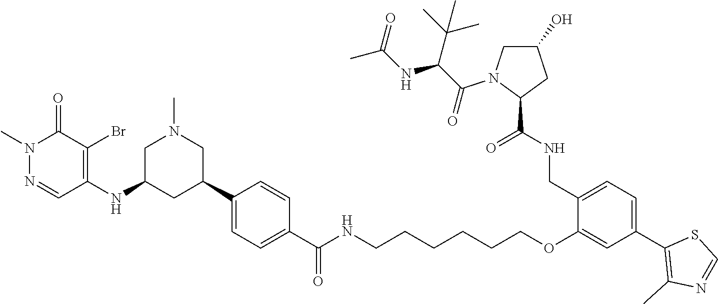 [SMILES: CC(=O)N[C@H](C(=O)N1C[C@H](O)C[C@H]1C(=O)NCc1ccc(-c2scnc2C)cc1OCCCCCCNC(=O)c1ccc([C@H]2C[C@@H](Nc3cnn(C)c(=O)c3Br)CN(C)C2)cc1)C(C)(C)C] |
| 407a | 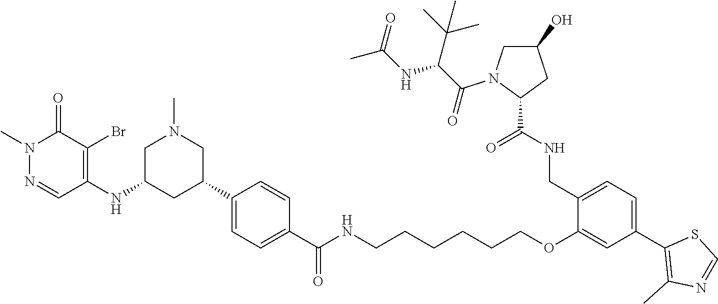 [SMILES: CC(=O)N[C@@H](C(=O)N1C[C@@H](O)C[C@@H]1C(=O)NCc1ccc(-c2scnc2C)cc1OCCCCCCNC(=O)c1ccc([C@@H]2C[C@H](Nc3cnn(C)c(=O)c3Br)CN(C)C2)cc1)C(C)(C)C] |
| 407b | 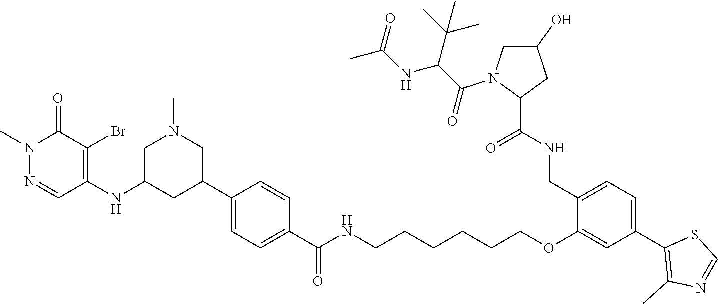 [SMILES: CC(=O)NC(C(=O)N1CC(O)CC1C(=O)NCc1ccc(-c2scnc2C)cc1OCCCCCCNC(=O)c1ccc(C2CC(Nc3cnn(C)c(=O)c3Br)CN(C)C2)cc1)C(C)(C)C] |
| 408 | 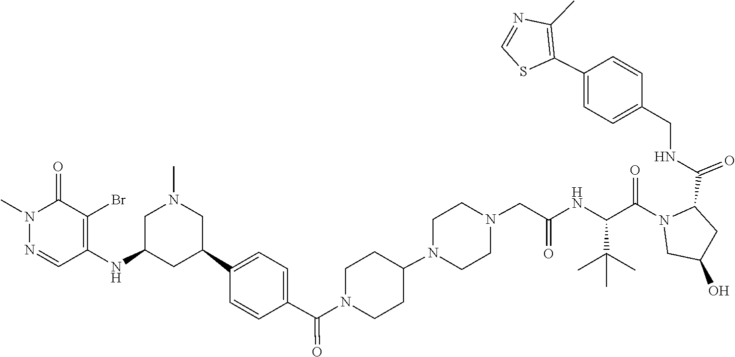 [SMILES: Cc1ncsc1-c1ccc(CNC(=O)[C@@H]2C[C@@H](O)CN2C(=O)[C@@H](NC(=O)CN2CCN(C3CCN(C(=O)c4ccc([C@H]5C[C@@H](Nc6cnn(C)c(=O)c6Br)CN(C)C5)cc4)CC3)CC2)C(C)(C)C)cc1] |
| 408a | 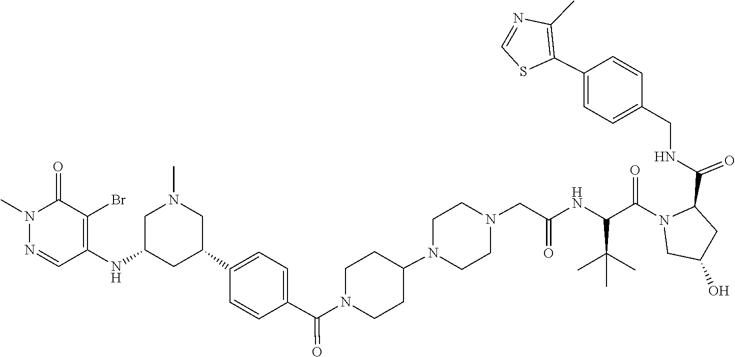 |
| 408b | 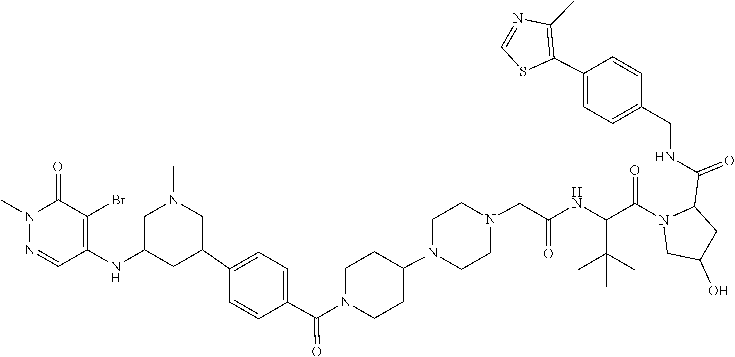 [SMILES: Cc1ncsc1-c1ccc(CNC(=O)C2CC(O)CN2C(=O)C(NC(=O)CN2CCN(C3CCN(C(=O)c4ccc(C5CC(Nc6cnn(C)c(=O)c6Br)CN(C)C5)cc4)CC3)CC2)C(C)(C)C)cc1] |
| 409 | 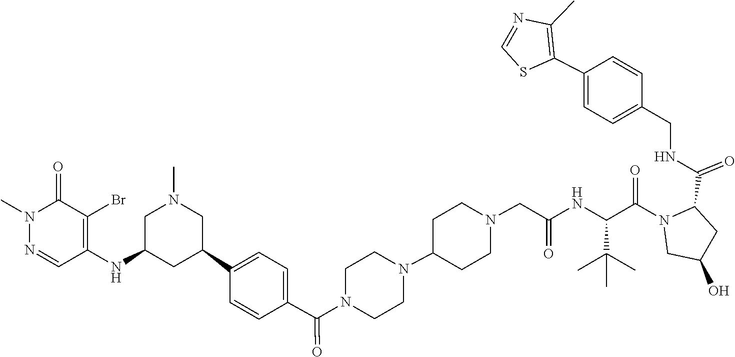 [SMILES: Cc1ncsc1-c1ccc(CNC(=O)[C@@H]2C[C@@H](O)CN2C(=O)[C@@H](NC(=O)CN2CCC(N3CCN(C(=O)c4ccc([C@H]5C[C@@H](Nc6cnn(C)c(=O)c6Br)CN(C)C5)cc4)CC3)CC2)C(C)(C)C)cc1] |
| 409a | 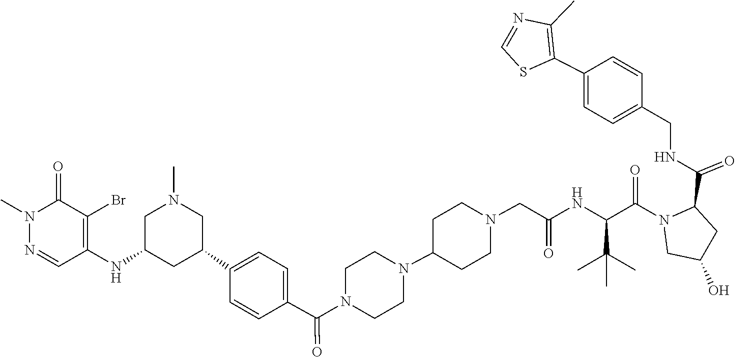 [SMILES: Cc1ncsc1-c1ccc(CNC(=O)[C@H]2C[C@H](O)CN2C(=O)[C@H](NC(=O)CN2CCC(N3CCN(C(=O)c4ccc([C@@H]5C[C@H](Nc6cnn(C)c(=O)c6Br)CN(C)C5)cc4)CC3)CC2)C(C)(C)C)cc1] |
| 409b | 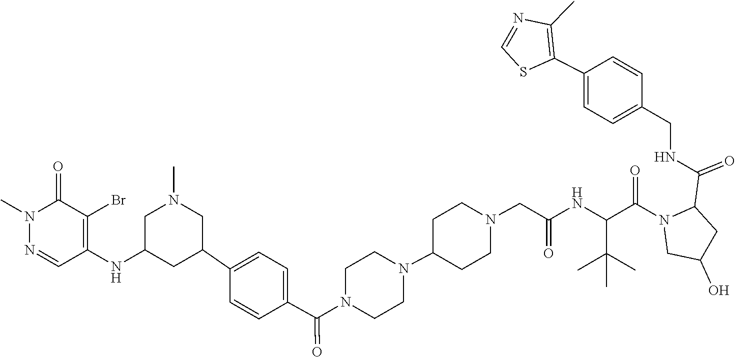 [SMILES: Cc1ncsc1-c1ccc(CNC(=O)C2CC(O)CN2C(=O)C(NC(=O)CN2CCC(N3CCN(C(=O)c4ccc(C5CC(Nc6cnn(C)c(=O)c6Br)CN(C)C5)cc4)CC3)CC2)C(C)(C)C)cc1] |
| 410 | 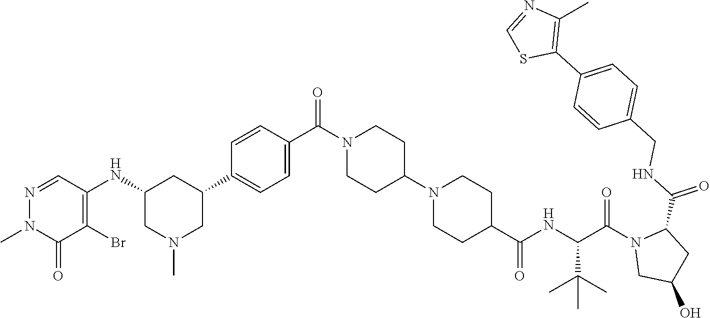 [SMILES: Cc1ncsc1-c1ccc(CNC(=O)[C@@H]2C[C@@H](O)CN2C(=O)[C@@H](NC(=O)C2CCN(C3CCN(C(=O)c4ccc([C@H]5C[C@@H](Nc6cnn(C)c(=O)c6Br)CN(C)C5)cc4)CC3)CC2)C(C)(C)C)cc1] |
| 410a | 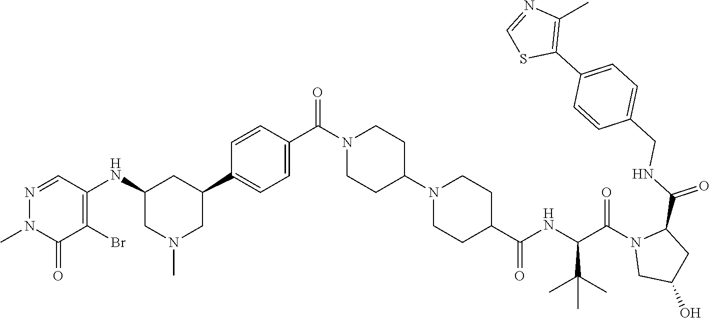 [SMILES: Cc1ncsc1-c1ccc(CNC(=O)[C@H]2C[C@H](O)CN2C(=O)[C@H](NC(=O)C2CCN(C3CCN(C(=O)c4ccc([C@@H]5C[C@H](Nc6cnn(C)c(=O)c6Br)CN(C)C5)cc4)CC3)CC2)C(C)(C)C)cc1] |
| 410b | 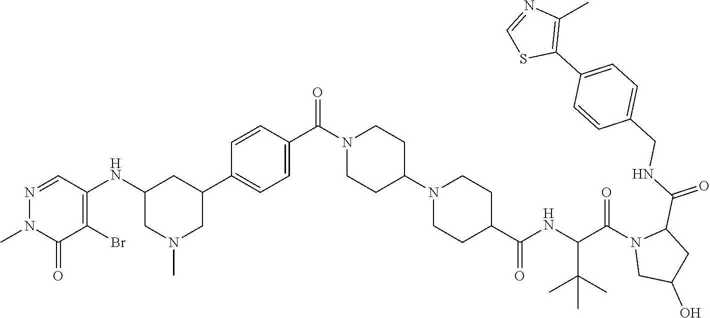 [SMILES: Cc1ncsc1-c1ccc(CNC(=O)C2CC(O)CN2C(=O)C(NC(=O)C2CCN(C3CCN(C(=O)c4ccc(C5CC(Nc6cnn(C)c(=O)c6Br)CN(C)C5)cc4)CC3)CC2)C(C)(C)C)cc1] |
| 411 | 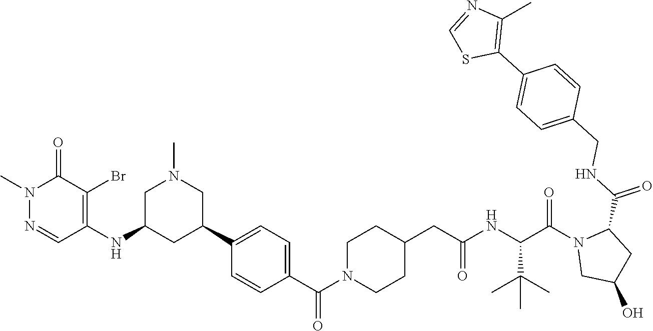 [SMILES: Cc1ncsc1-c1ccc(CNC(=O)[C@@H]2C[C@@H](O)CN2C(=O)[C@@H](NC(=O)CC2CCN(C(=O)c3ccc([C@H]4C[C@@H](Nc5cnn(C)c(=O)c5Br)CN(C)C4)cc3)CC2)C(C)(C)C)cc1] |
| 411a | 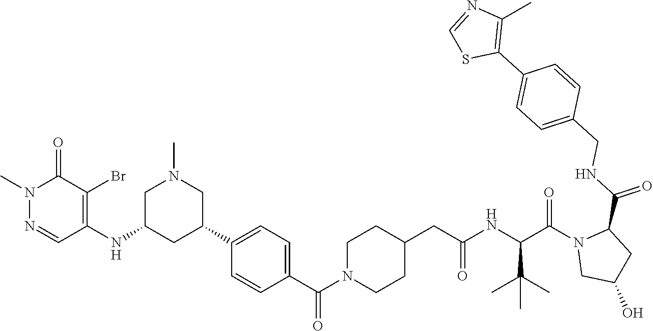 [SMILES: Cc1ncsc1-c1ccc(CNC(=O)[C@H]2C[C@H](O)CN2C(=O)[C@H](NC(=O)CC2CCN(C(=O)c3ccc([C@@H]4C[C@H](Nc5cnn(C)c(=O)c5Br)CN(C)C4)cc3)CC2)C(C)(C)C)cc1] |
| 411b | 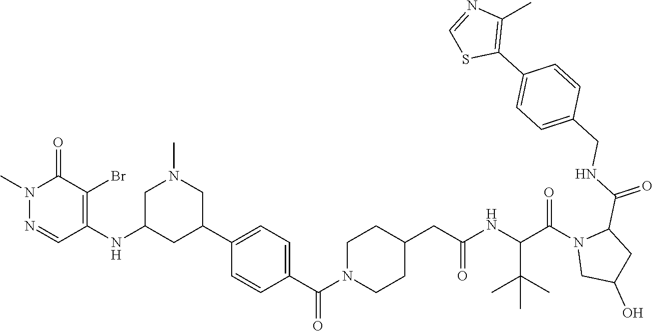 [SMILES: Cc1ncsc1-c1ccc(CNC(=O)C2CC(O)CN2C(=O)C(NC(=O)CC2CCN(C(=O)c3ccc(C4CC(Nc5cnn(C)c(=O)c5Br)CN(C)C4)cc3)CC2)C(C)(C)C)cc1] |
| 412 | 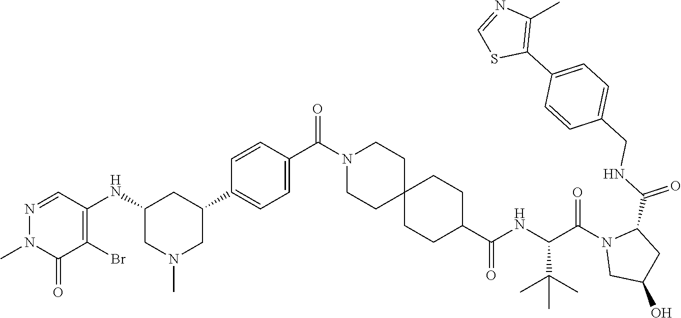 [SMILES: Cc1ncsc1-c1ccc(CNC(=O)[C@@H]2C[C@@H](O)CN2C(=O)[C@@H](NC(=O)C2CCC3(CC2)CCN(C(=O)c2ccc([C@H]4C[C@@H](Nc5cnn(C)c(=O)c5Br)CN(C)C4)cc2)CC3)C(C)(C)C)cc1] |
| 412a | 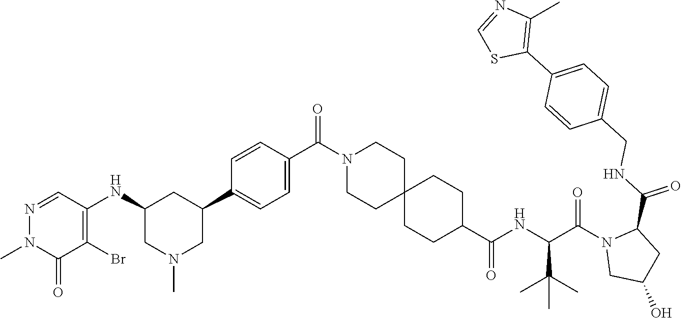 [SMILES: Cc1ncsc1-c1ccc(CNC(=O)[C@H]2C[C@H](O)CN2C(=O)[C@H](NC(=O)C2CCC3(CC2)CCN(C(=O)c2ccc([C@@H]4C[C@H](Nc5cnn(C)c(=O)c5Br)CN(C)C4)cc2)CC3)C(C)(C)C)cc1] |
| 412b | 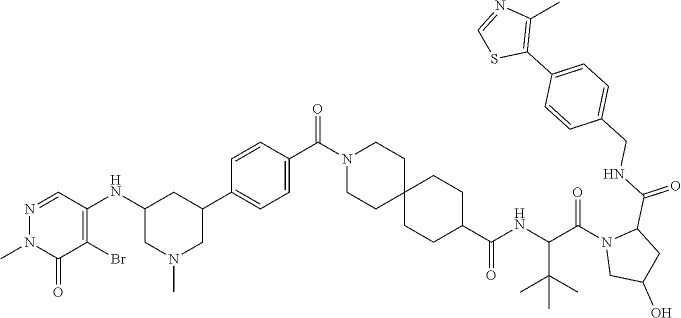 [SMILES: Cc1ncsc1-c1ccc(CNC(=O)C2CC(O)CN2C(=O)C(NC(=O)C2CCC3(CC2)CCN(C(=O)c2ccc(C4CC(Nc5cnn(C)c(=O)c5Br)CN(C)C4)cc2)CC3)C(C)(C)C)cc1] |
| 413 | 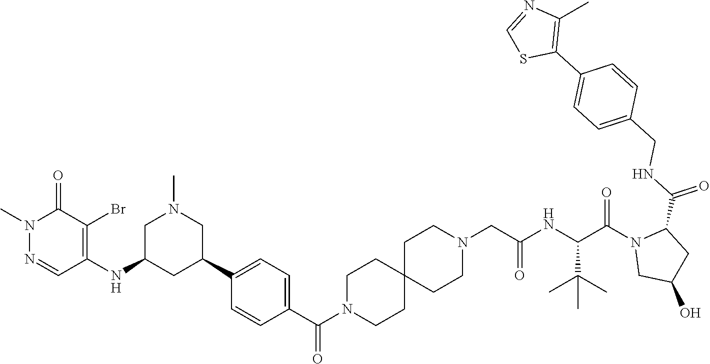 [SMILES: Cc1ncsc1-c1ccc(CNC(=O)[C@@H]2C[C@@H](O)CN2C(=O)[C@@H](NC(=O)CN2CCC3(CC2)CCN(C(=O)c2ccc([C@H]4C[C@@H](Nc5cnn(C)c(=O)c5Br)CN(C)C4)cc2)CC3)C(C)(C)C)cc1] |
| 413a | 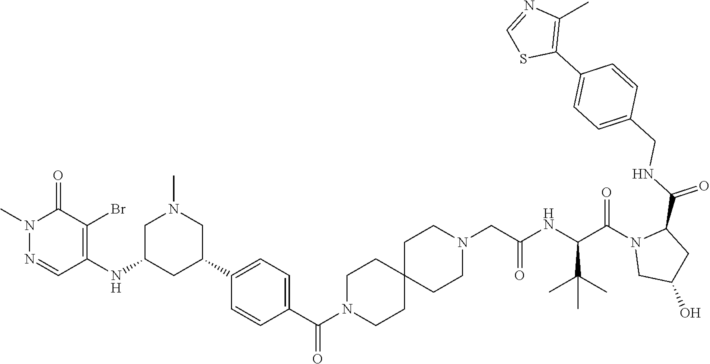 [SMILES: Cc1ncsc1-c1ccc(CNC(=O)[C@H]2C[C@H](O)CN2C(=O)[C@H](NC(=O)CN2CCC3(CC2)CCN(C(=O)c2ccc([C@@H]4C[C@H](Nc5cnn(C)c(=O)c5Br)CN(C)C4)cc2)CC3)C(C)(C)C)cc1] |
| 413b | 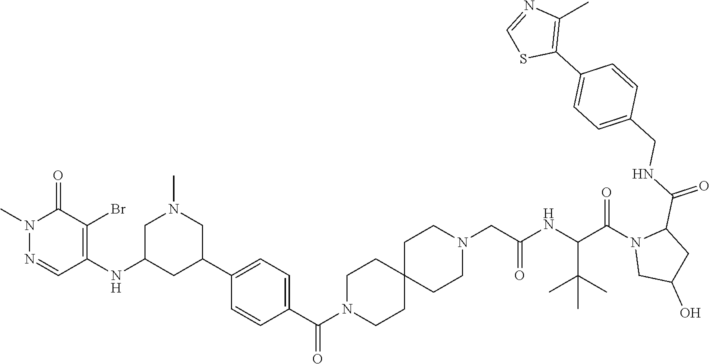 [SMILES: Cc1ncsc1-c1ccc(CNC(=O)C2CC(O)CN2C(=O)C(NC(=O)CN2CCC3(CC2)CCN(C(=O)c2ccc(C4CC(Nc5cnn(C)c(=O)c5Br)CN(C)C4)cc2)CC3)C(C)(C)C)cc1] |
| 414 | 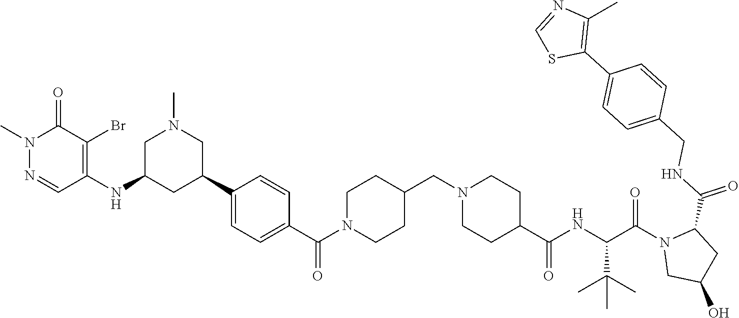 [SMILES: Cc1ncsc1-c1ccc(CNC(=O)[C@@H]2C[C@@H](O)CN2C(=O)[C@@H](NC(=O)C2CCN(CC3CCN(C(=O)c4ccc([C@H]5C[C@@H](Nc6cnn(C)c(=O)c6Br)CN(C)C5)cc4)CC3)CC2)C(C)(C)C)cc1] |
| 414a | 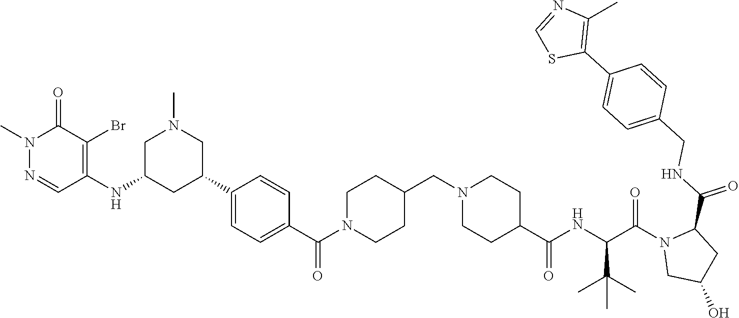 [SMILES: Cc1ncsc1-c1ccc(CNC(=O)[C@H]2C[C@H](O)CN2C(=O)[C@H](NC(=O)C2CCN(CC3CCN(C(=O)c4ccc([C@@H]5C[C@H](Nc6cnn(C)c(=O)c6Br)CN(C)C5)cc4)CC3)CC2)C(C)(C)C)cc1] |
| 414b | 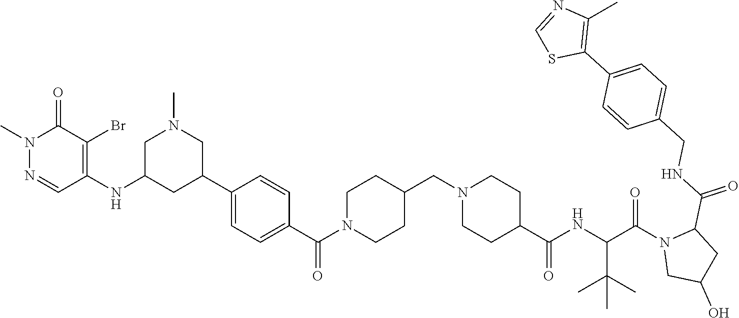 [SMILES: Cc1ncsc1-c1ccc(CNC(=O)C2CC(O)CN2C(=O)C(NC(=O)C2CCN(CC3CCN(C(=O)c4ccc(C5CC(Nc6cnn(C)c(=O)c6Br)CN(C)C5)cc4)CC3)CC2)C(C)(C)C)cc1] |
| 415 | 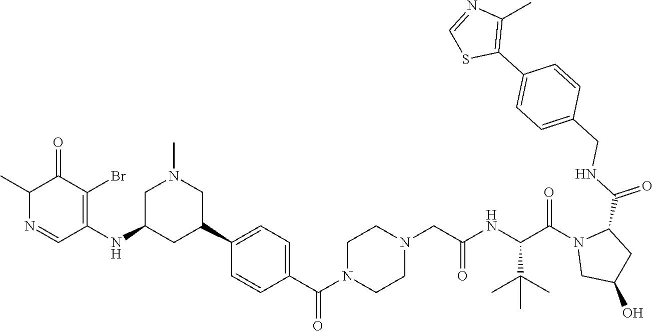 |
| 415a | 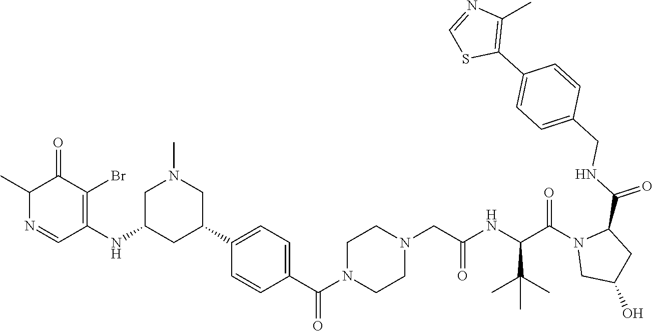 [SMILES: Cc1ncsc1-c1ccc(CNC(=O)[C@H]2C[C@H](O)CN2C(=O)[C@H](NC(=O)CN2CCN(C(=O)c3ccc([C@@H]4C[C@H](NC5=C(Br)C(=O)C(C)N=C5)CN(C)C4)cc3)CC2)C(C)(C)C)cc1] |
| 415b | 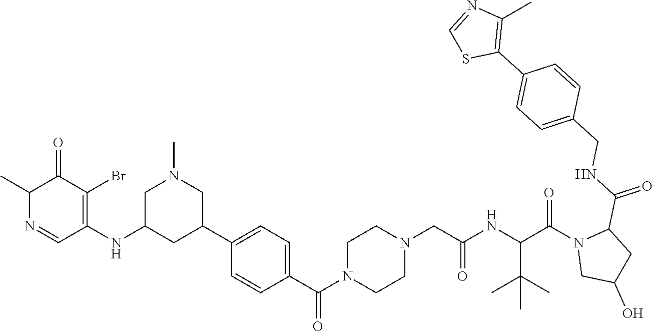 [SMILES: Cc1ncsc1-c1ccc(CNC(=O)C2CC(O)CN2C(=O)C(NC(=O)CN2CCN(C(=O)c3ccc(C4CC(NC5=C(Br)C(=O)C(C)N=C5)CN(C)C4)cc3)CC2)C(C)(C)C)cc1] |
| 416 | 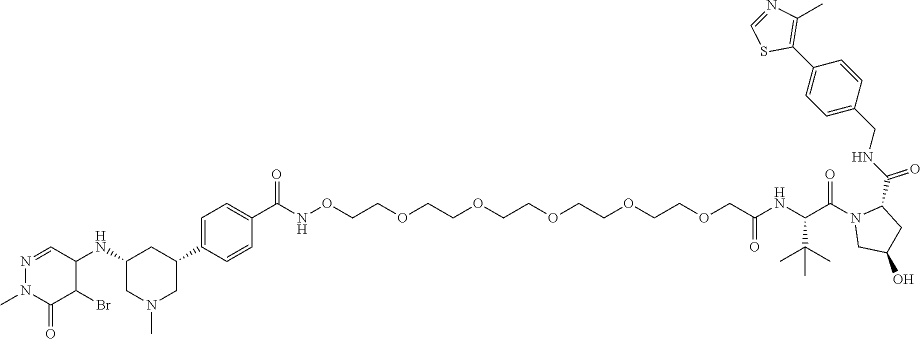 [SMILES: Cc1ncsc1-c1ccc(CNC(=O)[C@@H]2C[C@@H](O)CN2C(=O)[C@@H](NC(=O)COCCOCCOCCOCCOCCONC(=O)c2ccc([C@H]3C[C@@H](NC4C=NN(C)C(=O)C4Br)CN(C)C3)cc2)C(C)(C)C)cc1] |
| 416a | 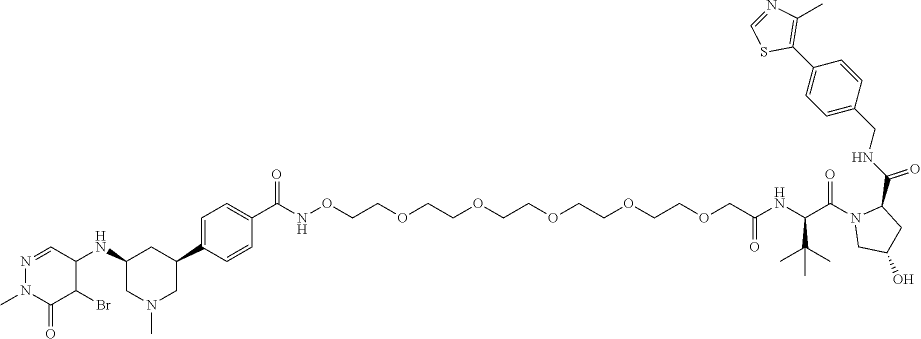 [SMILES: Cc1ncsc1-c1ccc(CNC(=O)[C@H]2C[C@H](O)CN2C(=O)[C@H](NC(=O)COCCOCCOCCOCCOCCONC(=O)c2ccc([C@@H]3C[C@H](NC4C=NN(C)C(=O)C4Br)CN(C)C3)cc2)C(C)(C)C)cc1] |
| 416b | 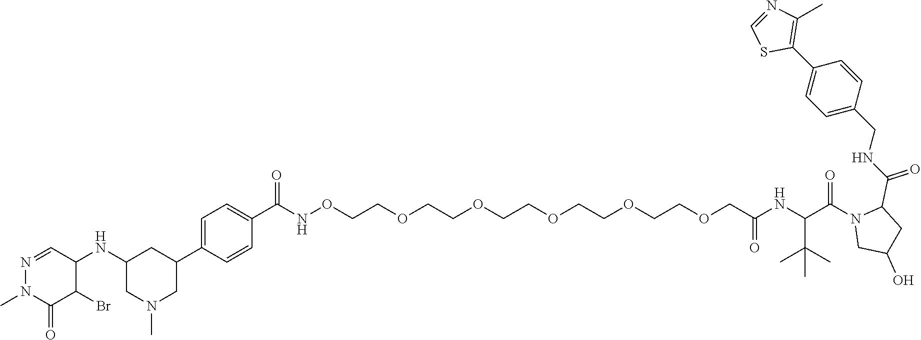 [SMILES: Cc1ncsc1-c1ccc(CNC(=O)C2CC(O)CN2C(=O)C(NC(=O)COCCOCCOCCOCCOCCONC(=O)c2ccc(C3CC(NC4C=NN(C)C(=O)C4Br)CN(C)C3)cc2)C(C)(C)C)cc1] |
| 417 | 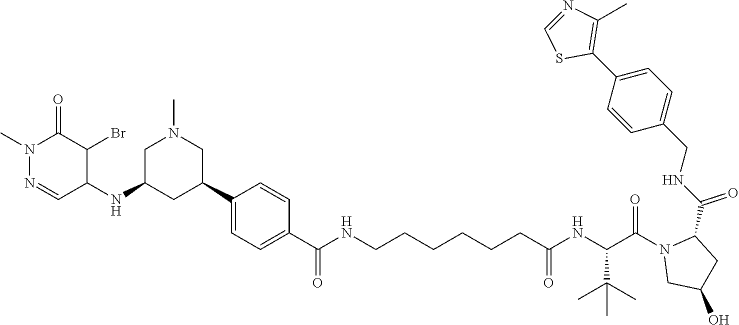 [SMILES: Cc1ncsc1-c1ccc(CNC(=O)[C@@H]2C[C@@H](O)CN2C(=O)[C@@H](NC(=O)CCCCCCNC(=O)c2ccc([C@H]3C[C@@H](NC4C=NN(C)C(=O)C4Br)CN(C)C3)cc2)C(C)(C)C)cc1] |
| 417a | 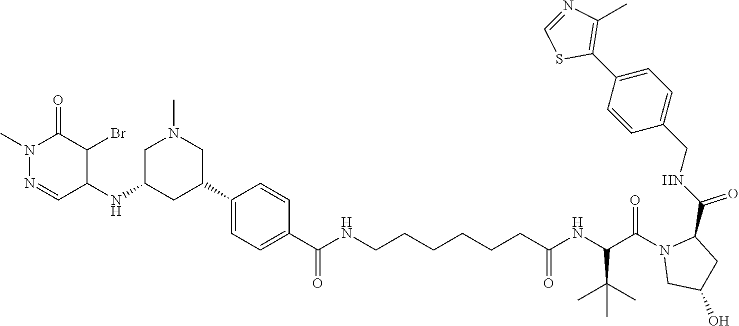 |
| 417b | 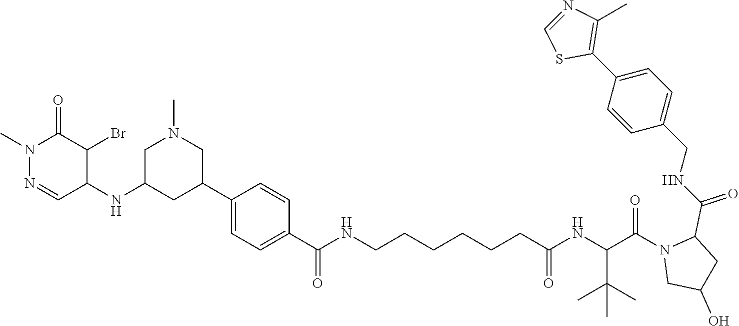 [SMILES: Cc1ncsc1-c1ccc(CNC(=O)C2CC(O)CN2C(=O)C(NC(=O)CCCCCCNC(=O)c2ccc(C3CC(NC4C=NN(C)C(=O)C4Br)CN(C)C3)cc2)C(C)(C)C)cc1] |
| 418 | 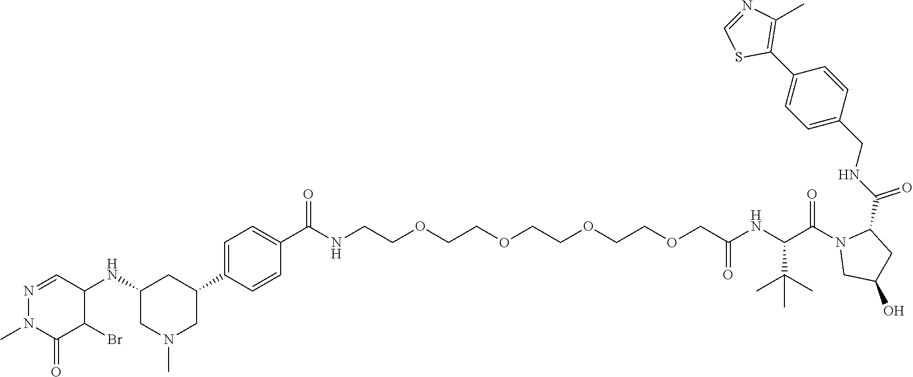 [SMILES: Cc1ncsc1-c1ccc(CNC(=O)[C@@H]2C[C@@H](O)CN2C(=O)[C@@H](NC(=O)COCCOCCOCCOCCNC(=O)c2ccc([C@H]3C[C@@H](NC4C=NN(C)C(=O)C4Br)CN(C)C3)cc2)C(C)(C)C)cc1] |
| 418a | 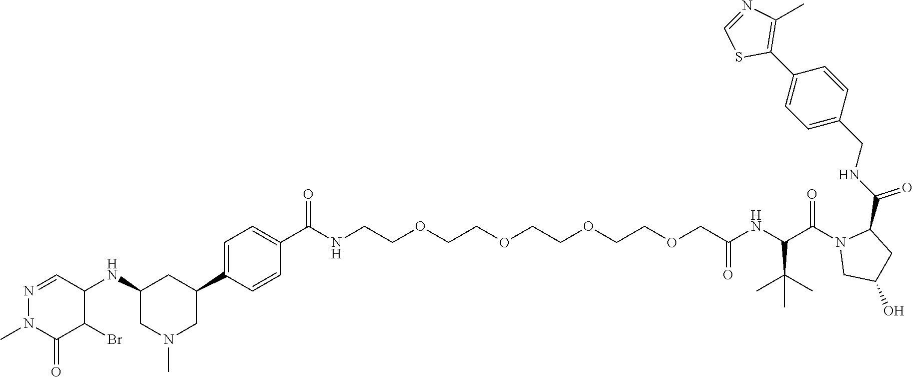 |
| 418b | 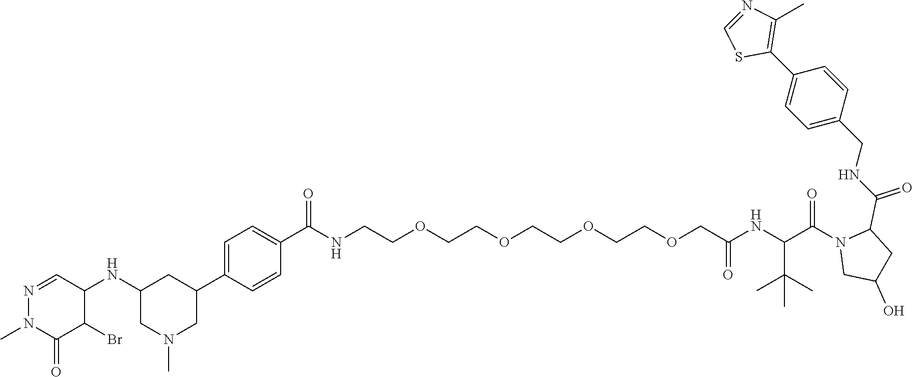 [SMILES: Cc1ncsc1-c1ccc(CNC(=O)C2CC(O)CN2C(=O)C(NC(=O)COCCOCCOCCOCCNC(=O)c2ccc(C3CC(NC4C=NN(C)C(=O)C4Br)CN(C)C3)cc2)C(C)(C)C)cc1] |
| 419 | 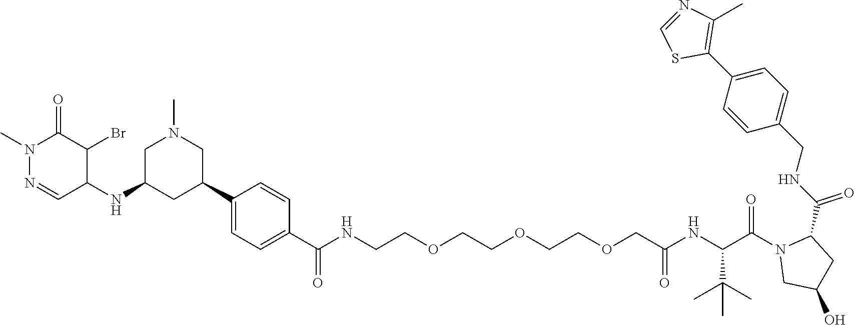 [SMILES: Cc1ncsc1-c1ccc(CNC(=O)[C@@H]2C[C@@H](O)CN2C(=O)[C@@H](NC(=O)COCCOCCOCCNC(=O)c2ccc([C@H]3C[C@@H](NC4C=NN(C)C(=O)C4Br)CN(C)C3)cc2)C(C)(C)C)cc1] |
| 419a | 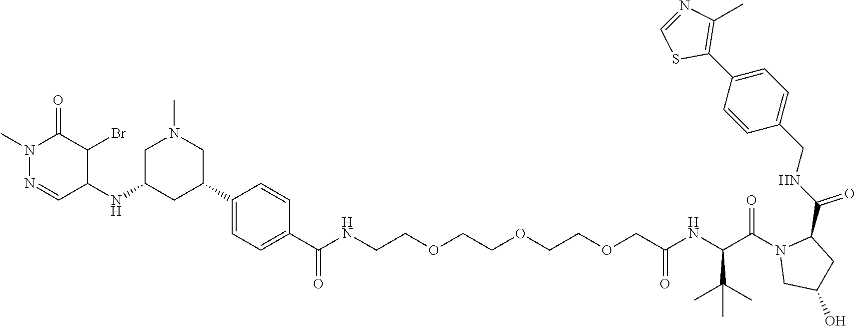 [SMILES: Cc1ncsc1-c1ccc(CNC(=O)[C@H]2C[C@H](O)CN2C(=O)[C@H](NC(=O)COCCOCCOCCNC(=O)c2ccc([C@@H]3C[C@H](NC4C=NN(C)C(=O)C4Br)CN(C)C3)cc2)C(C)(C)C)cc1] |
| 419b | 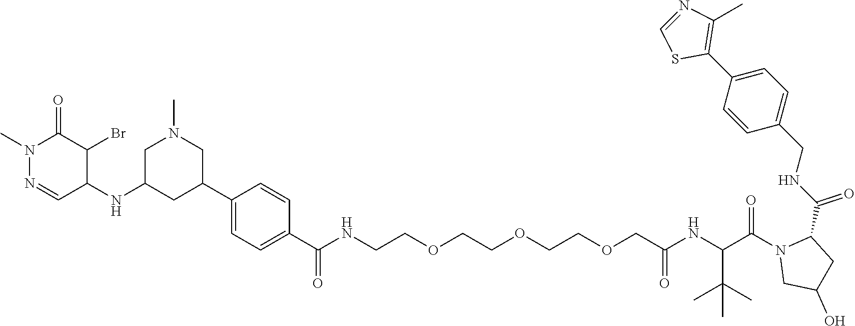 [SMILES: Cc1ncsc1-c1ccc(CNC(=O)[C@@H]2CC(O)CN2C(=O)C(NC(=O)COCCOCCOCCNC(=O)c2ccc(C3CC(NC4C=NN(C)C(=O)C4Br)CN(C)C3)cc2)C(C)(C)C)cc1] |
| 420 | 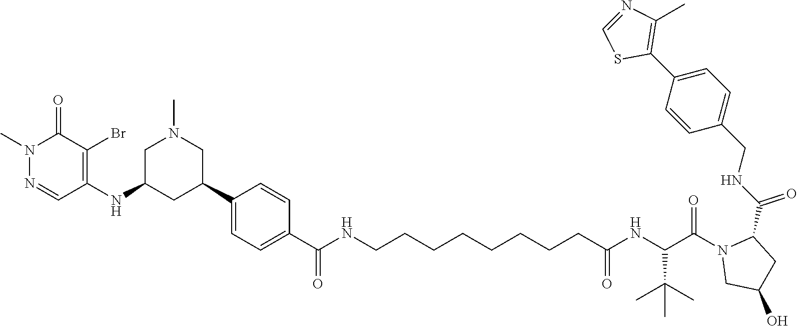 |
| 420a | 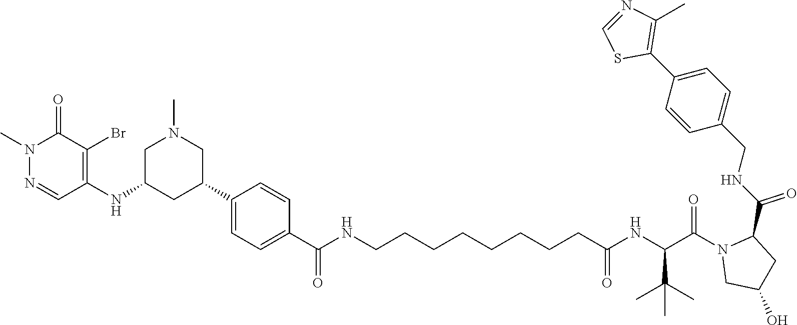 [SMILES: Cc1ncsc1-c1ccc(CNC(=O)[C@H]2C[C@H](O)CN2C(=O)[C@H](NC(=O)CCCCCCCCNC(=O)c2ccc([C@@H]3C[C@H](Nc4cnn(C)c(=O)c4Br)CN(C)C3)cc2)C(C)(C)C)cc1] |
| 420b | 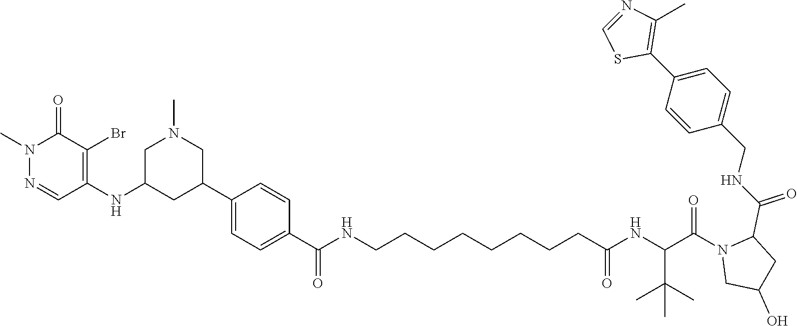 [SMILES: Cc1ncsc1-c1ccc(CNC(=O)C2CC(O)CN2C(=O)C(NC(=O)CCCCCCCCNC(=O)c2ccc(C3CC(Nc4cnn(C)c(=O)c4Br)CN(C)C3)cc2)C(C)(C)C)cc1] |
| 421 | 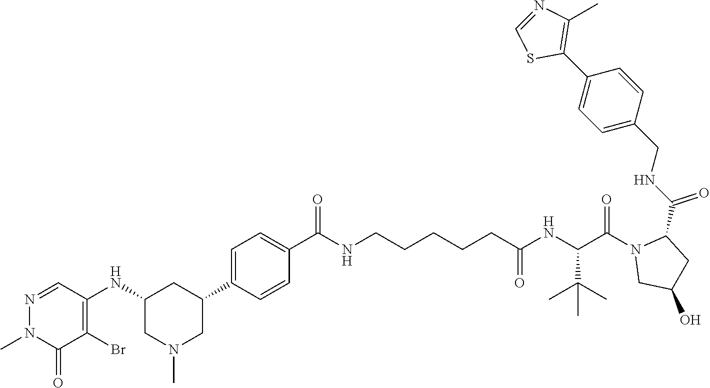 [SMILES: Cc1ncsc1-c1ccc(CNC(=O)[C@@H]2C[C@@H](O)CN2C(=O)[C@@H](NC(=O)CCCCCNC(=O)c2ccc([C@H]3C[C@@H](Nc4cnn(C)c(=O)c4Br)CN(C)C3)cc2)C(C)(C)C)cc1] |
| 421a | 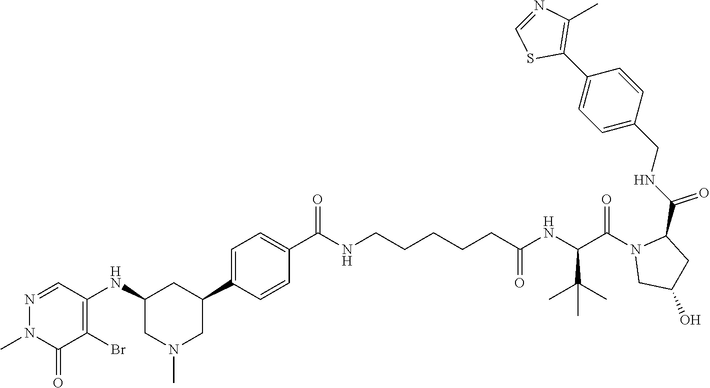 [SMILES: Cc1ncsc1-c1ccc(CNC(=O)[C@H]2C[C@H](O)CN2C(=O)[C@H](NC(=O)CCCCCNC(=O)c2ccc([C@@H]3C[C@H](Nc4cnn(C)c(=O)c4Br)CN(C)C3)cc2)C(C)(C)C)cc1] |
| 421b | 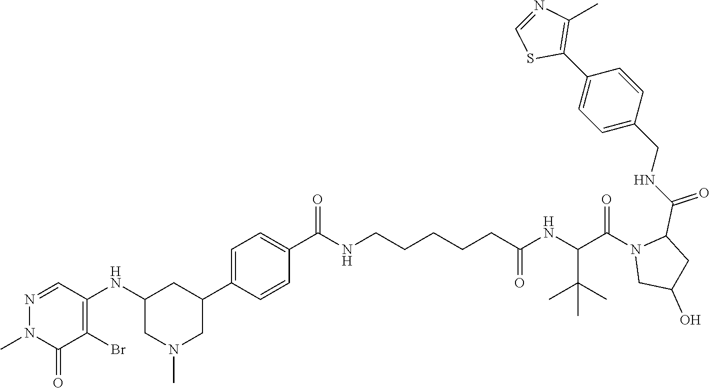 [SMILES: Cc1ncsc1-c1ccc(CNC(=O)C2CC(O)CN2C(=O)C(NC(=O)CCCCCNC(=O)c2ccc(C3CC(Nc4cnn(C)c(=O)c4Br)CN(C)C3)cc2)C(C)(C)C)cc1] |
| 422 | 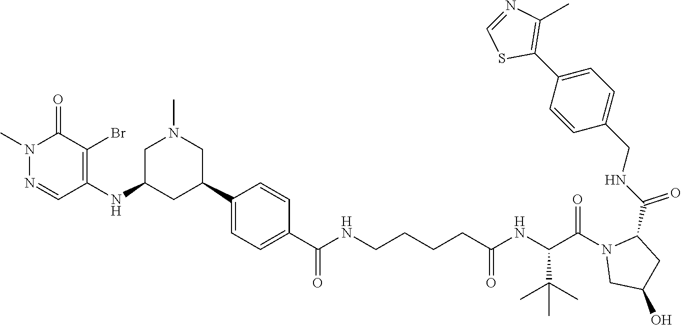 [SMILES: Cc1ncsc1-c1ccc(CNC(=O)[C@@H]2C[C@@H](O)CN2C(=O)[C@@H](NC(=O)CCCCNC(=O)c2ccc([C@H]3C[C@@H](Nc4cnn(C)c(=O)c4Br)CN(C)C3)cc2)C(C)(C)C)cc1] |
| 422a | 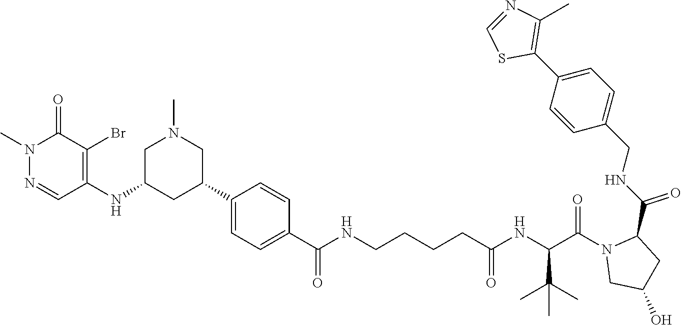 [SMILES: Cc1ncsc1-c1ccc(CNC(=O)[C@H]2C[C@H](O)CN2C(=O)[C@H](NC(=O)CCCCNC(=O)c2ccc([C@@H]3C[C@H](Nc4cnn(C)c(=O)c4Br)CN(C)C3)cc2)C(C)(C)C)cc1] |
| 422b | 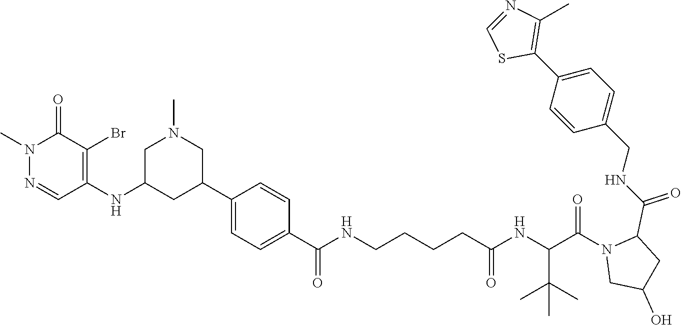 [SMILES: Cc1ncsc1-c1ccc(CNC(=O)C2CC(O)CN2C(=O)C(NC(=O)CCCCNC(=O)c2ccc(C3CC(Nc4cnn(C)c(=O)c4Br)CN(C)C3)cc2)C(C)(C)C)cc1] |
| 423 | 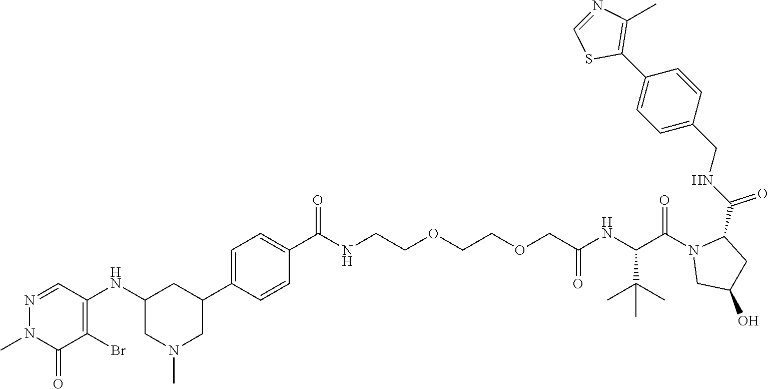 [SMILES: Cc1ncsc1-c1ccc(CNC(=O)[C@@H]2C[C@@H](O)CN2C(=O)[C@@H](NC(=O)COCCOCCNC(=O)c2ccc(C3CC(Nc4cnn(C)c(=O)c4Br)CN(C)C3)cc2)C(C)(C)C)cc1] |
| 423a | 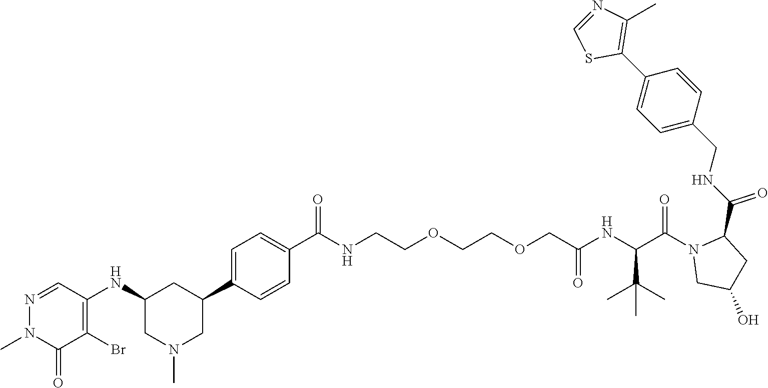 [SMILES: Cc1ncsc1-c1ccc(CNC(=O)[C@H]2C[C@H](O)CN2C(=O)[C@H](NC(=O)COCCOCCNC(=O)c2ccc([C@@H]3C[C@H](Nc4cnn(C)c(=O)c4Br)CN(C)C3)cc2)C(C)(C)C)cc1] |
| 423b | 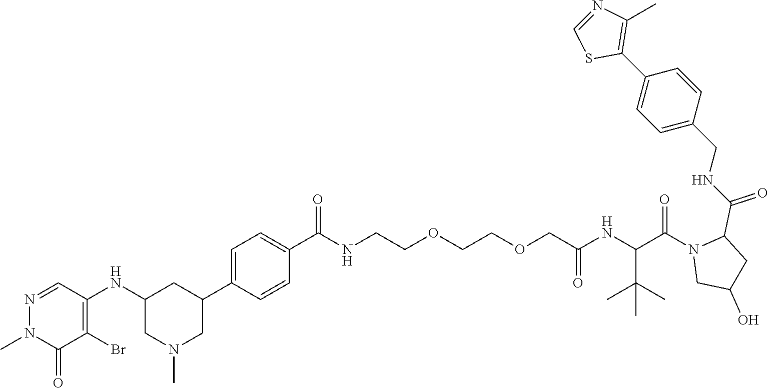 [SMILES: Cc1ncsc1-c1ccc(CNC(=O)C2CC(O)CN2C(=O)C(NC(=O)COCCOCCNC(=O)c2ccc(C3CC(Nc4cnn(C)c(=O)c4Br)CN(C)C3)cc2)C(C)(C)C)cc1] |
| 424 | 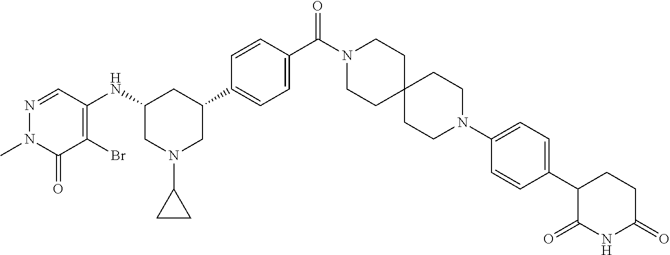 [SMILES: Cn1ncc(N[C@@H]2C[C@H](c3ccc(C(=O)N4CCC5(CC4)CCN(c4ccc(C6CCC(=O)NC6=O)cc4)CC5)cc3)CN(C3CC3)C2)c(Br)c1=O] |
| 424a | 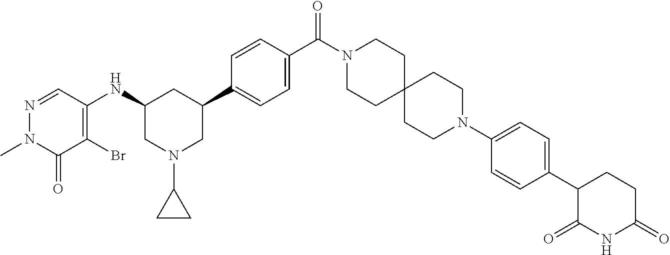 [SMILES: Cn1ncc(N[C@H]2C[C@@H](c3ccc(C(=O)N4CCC5(CC4)CCN(c4ccc(C6CCC(=O)NC6=O)cc4)CC5)cc3)CN(C3CC3)C2)c(Br)c1=O] |
| 424b | 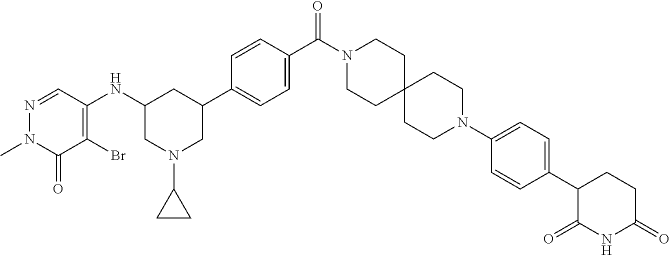 [SMILES: Cn1ncc(NC2CC(c3ccc(C(=O)N4CCC5(CC4)CCN(c4ccc(C6CCC(=O)NC6=O)cc4)CC5)cc3)CN(C3CC3)C2)c(Br)c1=O] |
| 425 | 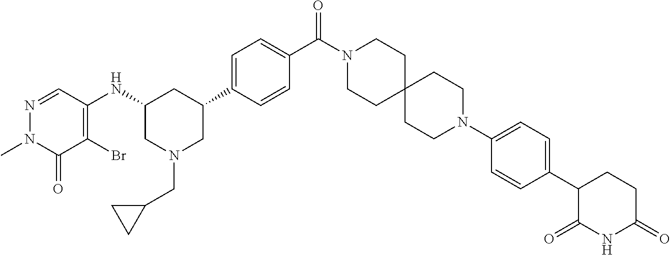 [SMILES: Cn1ncc(N[C@@H]2C[C@H](c3ccc(C(=O)N4CCC5(CC4)CCN(c4ccc(C6CCC(=O)NC6=O)cc4)CC5)cc3)CN(CC3CC3)C2)c(Br)c1=O] |
| 425a | 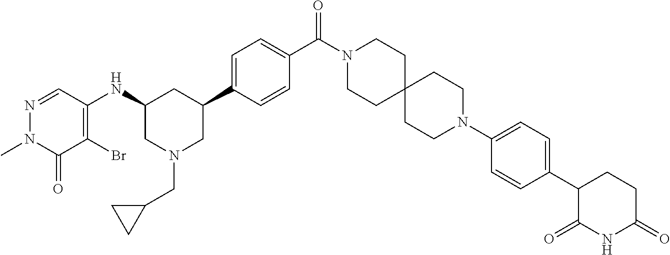 [SMILES: Cn1ncc(N[C@H]2C[C@@H](c3ccc(C(=O)N4CCC5(CC4)CCN(c4ccc(C6CCC(=O)NC6=O)cc4)CC5)cc3)CN(CC3CC3)C2)c(Br)c1=O] |
| 425b | 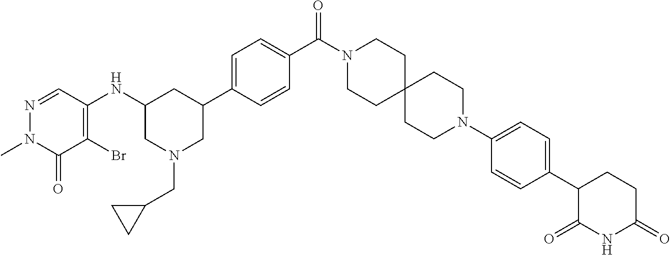 [SMILES: Cn1ncc(NC2CC(c3ccc(C(=O)N4CCC5(CC4)CCN(c4ccc(C6CCC(=O)NC6=O)cc4)CC5)cc3)CN(CC3CC3)C2)c(Br)c1=O] |
| 426 | 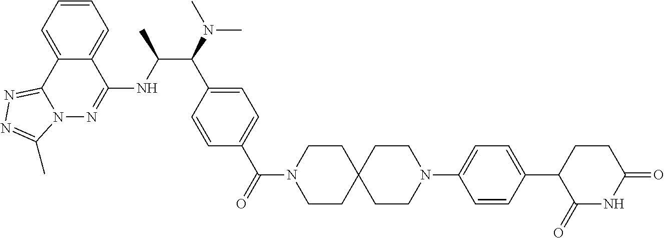 [SMILES: Cc1nnc2c3ccccc3c(N[C@@H](C)[C@H](c3ccc(C(=O)N4CCC5(CC4)CCN(c4ccc(C6CCC(=O)NC6=O)cc4)CC5)cc3)N(C)C)nn12] |
| 426a | 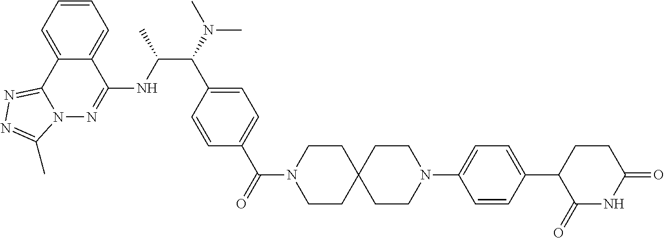 [SMILES: Cc1nnc2c3ccccc3c(N[C@H](C)[C@@H](c3ccc(C(=O)N4CCC5(CC4)CCN(c4ccc(C6CCC(=O)NC6=O)cc4)CC5)cc3)N(C)C)nn12] |
| 426b | 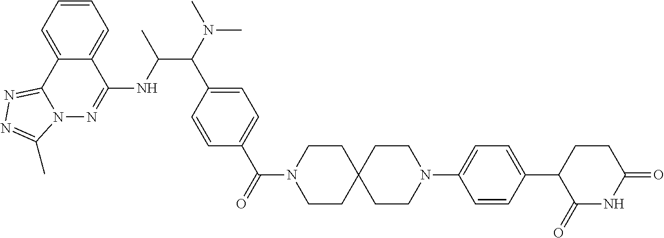 [SMILES: Cc1nnc2c3ccccc3c(NC(C)C(c3ccc(C(=O)N4CCC5(CC4)CCN(c4ccc(C6CCC(=O)NC6=O)cc4)CC5)cc3)N(C)C)nn12] |
| 427 | 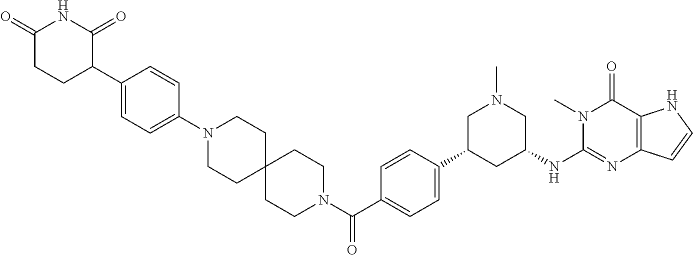 [SMILES: CN1C[C@H](Nc2nc3cc[nH]c3c(=O)n2C)C[C@H](c2ccc(C(=O)N3CCC4(CC3)CCN(c3ccc(C5CCC(=O)NC5=O)cc3)CC4)cc2)C1] |
| 427a | 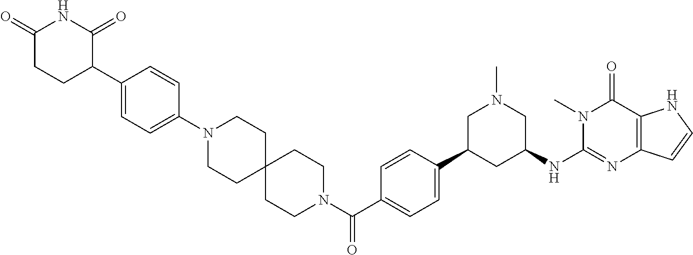 [SMILES: CN1C[C@@H](Nc2nc3cc[nH]c3c(=O)n2C)C[C@@H](c2ccc(C(=O)N3CCC4(CC3)CCN(c3ccc(C5CCC(=O)NC5=O)cc3)CC4)cc2)C1] |
| 427b | 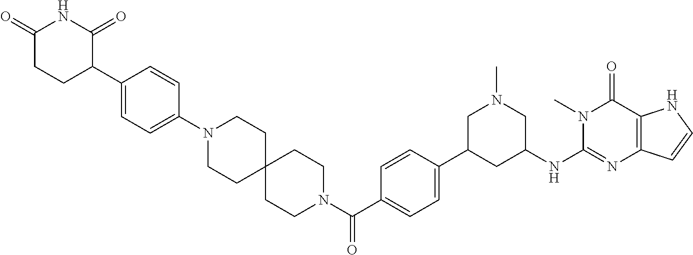 [SMILES: CN1CC(Nc2nc3cc[nH]c3c(=O)n2C)CC(c2ccc(C(=O)N3CCC4(CC3)CCN(c3ccc(C5CCC(=O)NC5=O)cc3)CC4)cc2)C1] |
| 428 | 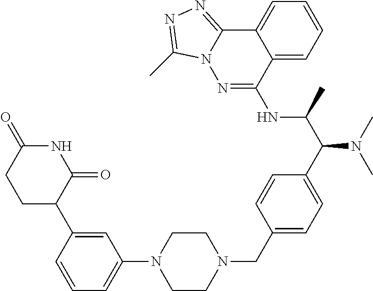 [SMILES: Cc1nnc2c3ccccc3c(N[C@@H](C)[C@H](c3ccc(CN4CCN(c5cccc(C6CCC(=O)NC6=O)c5)CC4)cc3)N(C)C)nn12] |
| 428a | 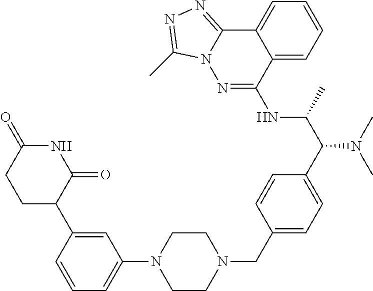 [SMILES: Cc1nnc2c3ccccc3c(N[C@H](C)[C@@H](c3ccc(CN4CCN(c5cccc(C6CCC(=O)NC6=O)c5)CC4)cc3)N(C)C)nn12] |
| 428b | 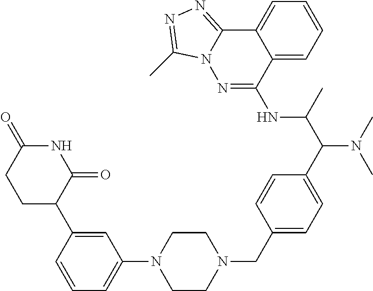 [SMILES: Cc1nnc2c3ccccc3c(NC(C)C(c3ccc(CN4CCN(c5cccc(C6CCC(=O)NC6=O)c5)CC4)cc3)N(C)C)nn12] |
| 429 | 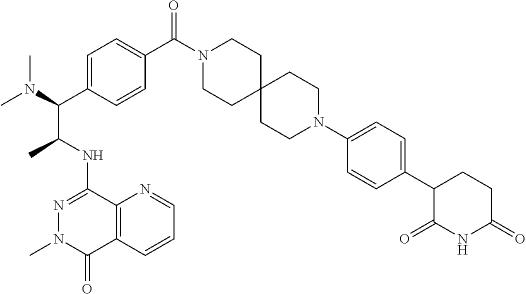 [SMILES: C[C@H](Nc1nn(C)c(=O)c2cccnc12)[C@H](c1ccc(C(=O)N2CCC3(CC2)CCN(c2ccc(C4CCC(=O)NC4=O)cc2)CC3)cc1)N(C)C] |
| 429a | 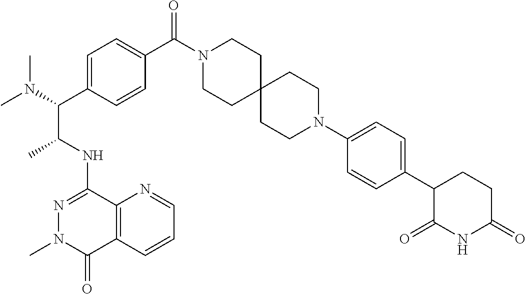 [SMILES: C[C@@H](Nc1nn(C)c(=O)c2cccnc12)[C@@H](c1ccc(C(=O)N2CCC3(CC2)CCN(c2ccc(C4CCC(=O)NC4=O)cc2)CC3)cc1)N(C)C] |
| 429b | 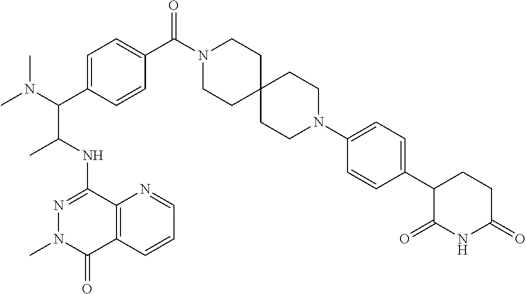 [SMILES: CC(Nc1nn(C)c(=O)c2cccnc12)C(c1ccc(C(=O)N2CCC3(CC2)CCN(c2ccc(C4CCC(=O)NC4=O)cc2)CC3)cc1)N(C)C] |
| 430 | 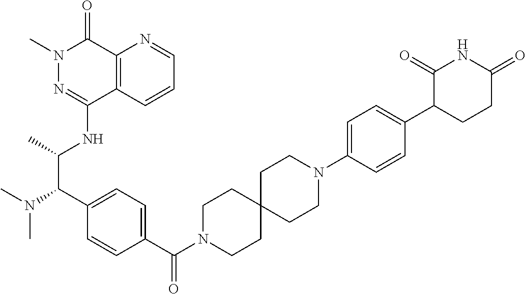 [SMILES: C[C@H](Nc1nn(C)c(=O)c2ncccc12)[C@H](c1ccc(C(=O)N2CCC3(CC2)CCN(c2ccc(C4CCC(=O)NC4=O)cc2)CC3)cc1)N(C)C] |
| 430a | 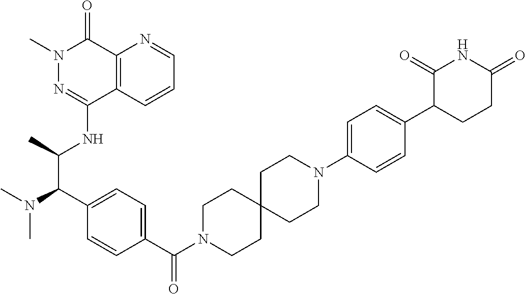 [SMILES: C[C@@H](Nc1nn(C)c(=O)c2ncccc12)[C@@H](c1ccc(C(=O)N2CCC3(CC2)CCN(c2ccc(C4CCC(=O)NC4=O)cc2)CC3)cc1)N(C)C] |
| 430b | 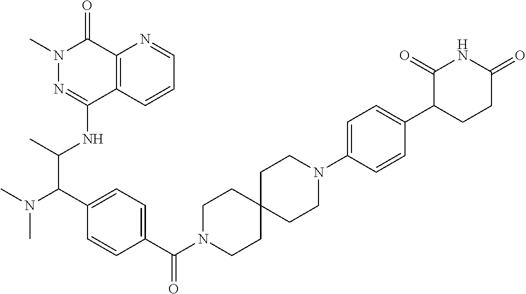 [SMILES: CC(Nc1nn(C)c(=O)c2ncccc12)C(c1ccc(C(=O)N2CCC3(CC2)CCN(c2ccc(C4CCC(=O)NC4=O)cc2)CC3)cc1)N(C)C] |
| 431 | 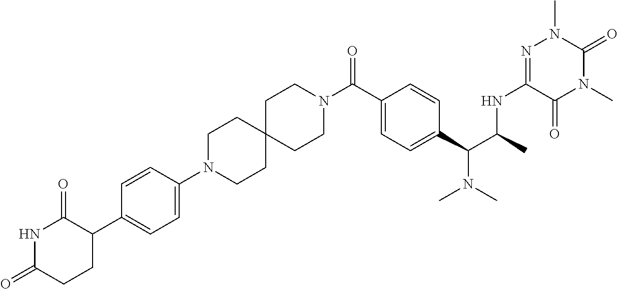 [SMILES: C[C@H](Nc1nn(C)c(=O)n(C)c1=O)[C@H](c1ccc(C(=O)N2CCC3(CC2)CCN(c2ccc(C4CCC(=O)NC4=O)cc2)CC3)cc1)N(C)C] |
| 431a | 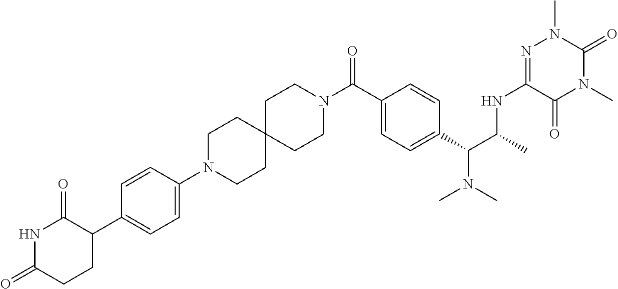 [SMILES: C[C@@H](Nc1nn(C)c(=O)n(C)c1=O)[C@@H](c1ccc(C(=O)N2CCC3(CC2)CCN(c2ccc(C4CCC(=O)NC4=O)cc2)CC3)cc1)N(C)C] |
| 431b | 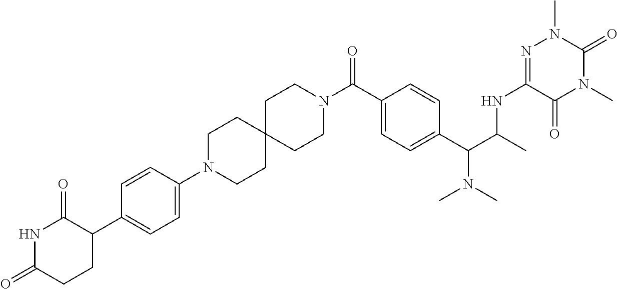 [SMILES: CC(Nc1nn(C)c(=O)n(C)c1=O)C(c1ccc(C(=O)N2CCC3(CC2)CCN(c2ccc(C4CCC(=O)NC4=O)cc2)CC3)cc1)N(C)C] |
| 432 | 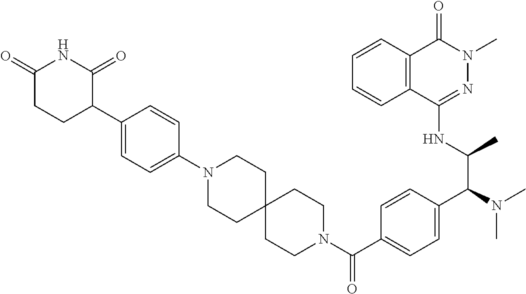 [SMILES: C[C@H](Nc1nn(C)c(=O)c2ccccc12)[C@H](c1ccc(C(=O)N2CCC3(CC2)CCN(c2ccc(C4CCC(=O)NC4=O)cc2)CC3)cc1)N(C)C] |
| 432a | 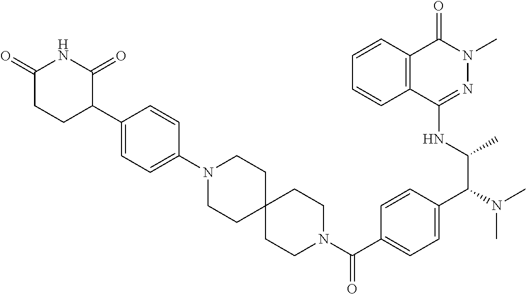 [SMILES: C[C@@H](Nc1nn(C)c(=O)c2ccccc12)[C@@H](c1ccc(C(=O)N2CCC3(CC2)CCN(c2ccc(C4CCC(=O)NC4=O)cc2)CC3)cc1)N(C)C] |
| 432b | 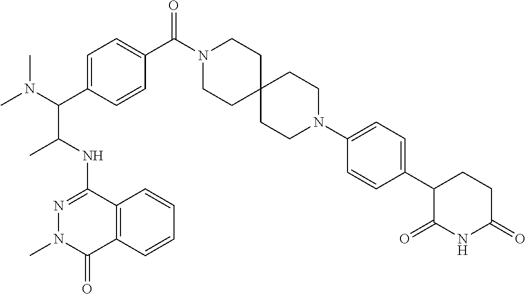 [SMILES: CC(Nc1nn(C)c(=O)c2ccccc12)C(c1ccc(C(=O)N2CCC3(CC2)CCN(c2ccc(C4CCC(=O)NC4=O)cc2)CC3)cc1)N(C)C] |
| 433 | 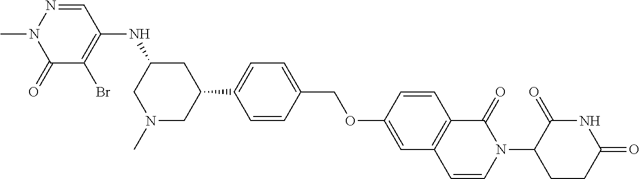 [SMILES: CN1C[C@H](Nc2cnn(C)c(=O)c2Br)C[C@H](c2ccc(COc3ccc4c(=O)n(C5CCC(=O)NC5=O)ccc4c3)cc2)C1] |
| 433a | 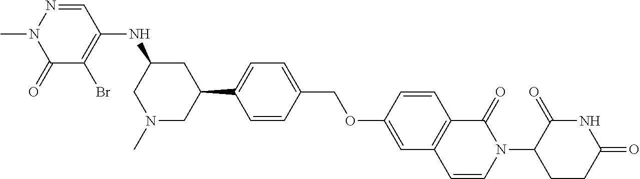 [SMILES: CN1C[C@@H](Nc2cnn(C)c(=O)c2Br)C[C@@H](c2ccc(COc3ccc4c(=O)n(C5CCC(=O)NC5=O)ccc4c3)cc2)C1] |
| 433b | 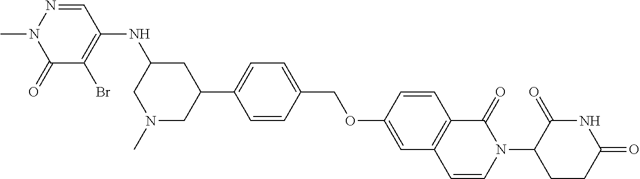 [SMILES: CN1CC(Nc2cnn(C)c(=O)c2Br)CC(c2ccc(COc3ccc4c(=O)n(C5CCC(=O)NC5=O)ccc4c3)cc2)C1] |
| 434 | 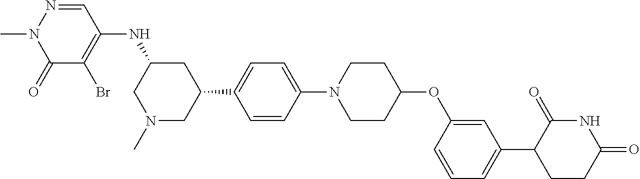 [SMILES: CN1C[C@H](Nc2cnn(C)c(=O)c2Br)C[C@H](c2ccc(N3CCC(Oc4cccc(C5CCC(=O)NC5=O)c4)CC3)cc2)C1] |
| 434a | 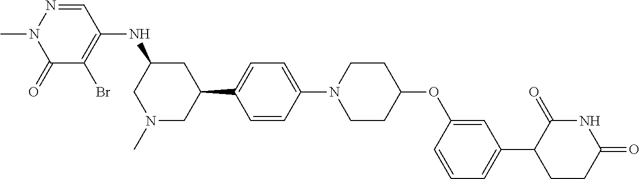 [SMILES: CN1C[C@@H](Nc2cnn(C)c(=O)c2Br)C[C@@H](c2ccc(N3CCC(Oc4cccc(C5CCC(=O)NC5=O)c4)CC3)cc2)C1] |
| 434b | 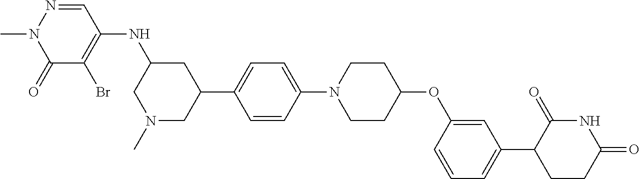 [SMILES: CN1CC(Nc2cnn(C)c(=O)c2Br)CC(c2ccc(N3CCC(Oc4cccc(C5CCC(=O)NC5=O)c4)CC3)cc2)C1] |
| 435 | 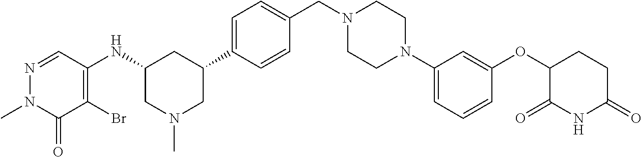 [SMILES: CN1C[C@H](Nc2cnn(C)c(=O)c2Br)C[C@H](c2ccc(CN3CCN(c4cccc(OC5CCC(=O)NC5=O)c4)CC3)cc2)C1] |
| 435a | 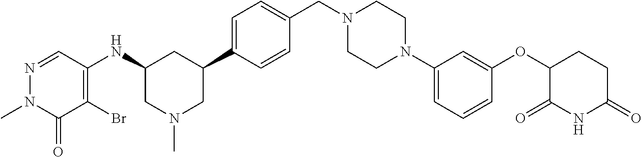 [SMILES: CN1C[C@@H](Nc2cnn(C)c(=O)c2Br)C[C@@H](c2ccc(CN3CCN(c4cccc(OC5CCC(=O)NC5=O)c4)CC3)cc2)C1] |
| 435b | 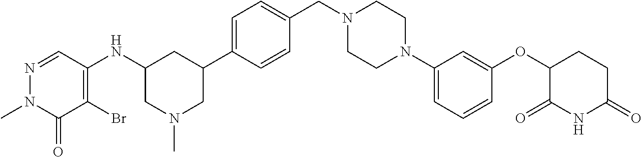 [SMILES: CN1CC(Nc2cnn(C)c(=O)c2Br)CC(c2ccc(CN3CCN(c4cccc(OC5CCC(=O)NC5=O)c4)CC3)cc2)C1] |
| 436 | 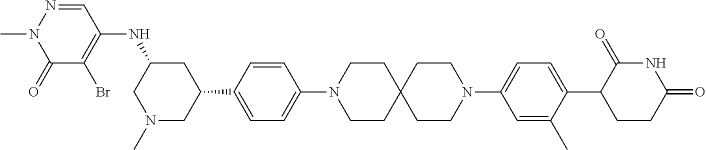 [SMILES: Cc1cc(N2CCC3(CCN(c4ccc([C@H]5C[C@@H](Nc6cnn(C)c(=O)c6Br)CN(C)C5)cc4)CC3)CC2)ccc1C1CCC(=O)NC1=O] |
| 436a | 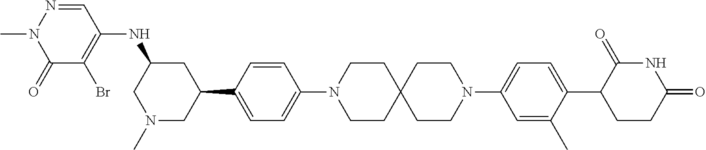 [SMILES: Cc1cc(N2CCC3(CCN(c4ccc([C@@H]5C[C@H](Nc6cnn(C)c(=O)c6Br)CN(C)C5)cc4)CC3)CC2)ccc1C1CCC(=O)NC1=O] |
| 436b | 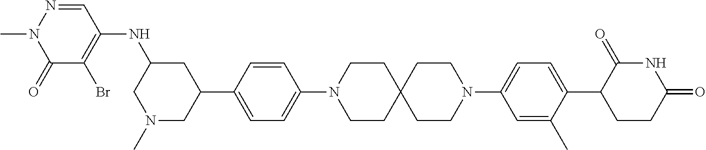 [SMILES: Cc1cc(N2CCC3(CCN(c4ccc(C5CC(Nc6cnn(C)c(=O)c6Br)CN(C)C5)cc4)CC3)CC2)ccc1C1CCC(=O)NC1=O] |
| 437/437-2 | 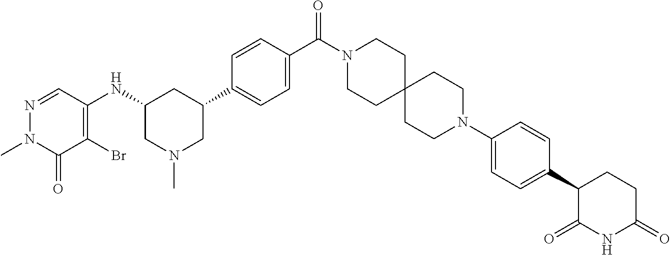 [SMILES: CN1C[C@H](Nc2cnn(C)c(=O)c2Br)C[C@H](c2ccc(C(=O)N3CCC4(CC3)CCN(c3ccc([C@H]5CCC(=O)NC5=O)cc3)CC4)cc2)C1] |
| 437/437-2 | 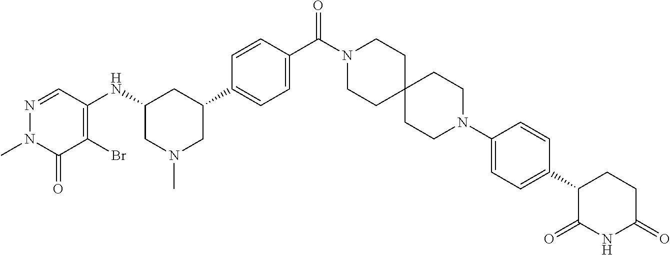 [SMILES: CN1C[C@H](Nc2cnn(C)c(=O)c2Br)C[C@H](c2ccc(C(=O)N3CCC4(CC3)CCN(c3ccc([C@@H]5CCC(=O)NC5=O)cc3)CC4)cc2)C1] |
| 437a | 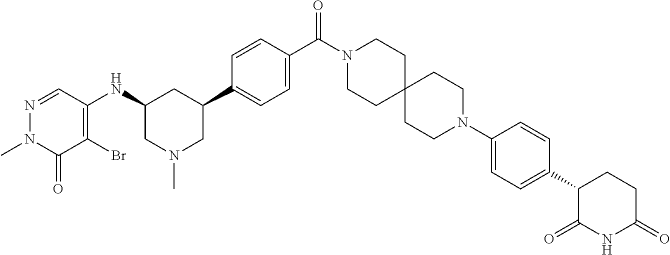 [SMILES: CN1C[C@@H](Nc2cnn(C)c(=O)c2Br)C[C@@H](c2ccc(C(=O)N3CCC4(CC3)CCN(c3ccc([C@@H]5CCC(=O)NC5=O)cc3)CC4)cc2)C1] |
| 437b | 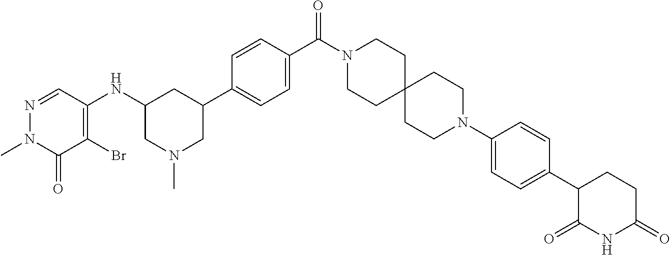 [SMILES: CN1CC(Nc2cnn(C)c(=O)c2Br)CC(c2ccc(C(=O)N3CCC4(CC3)CCN(c3ccc(C5CCC(=O)NC5=O)cc3)CC4)cc2)C1] |

In some embodiments, the present disclosure provides compounds selected from Table 2, or a pharmaceutically acceptable salt thereof.

TABLE 2

| Compound | |
|---|---|
| No. | Structure |
| | 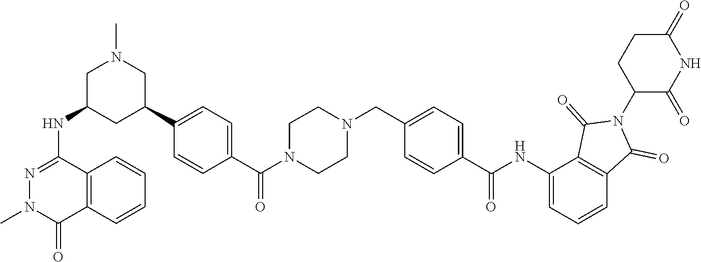 [SMILES: CN1C[C@H](Nc2nn(C)c(=O)c3ccccc23)C[C@H](c2ccc(C(=O)N3CCN(Cc4ccc(C(=O)Nc5cccc6c5C(=O)N(C5CCC(=O)NC5=O)C6=O)cc4)CC3)cc2)C1] |
| | 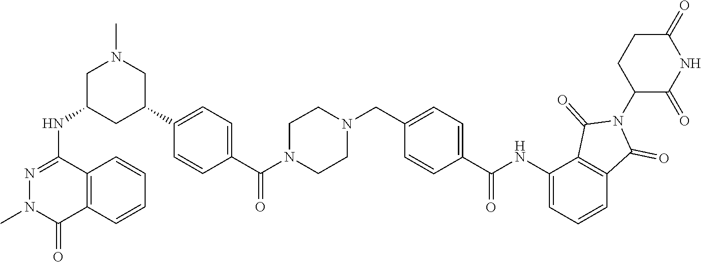 [SMILES: CN1C[C@@H](Nc2nn(C)c(=O)c3ccccc23)C[C@@H](c2ccc(C(=O)N3CCN(Cc4ccc(C(=O)Nc5cccc6c5C(=O)N(C5CCC(=O)NC5=O)C6=O)cc4)CC3)cc2)C1] |
| | 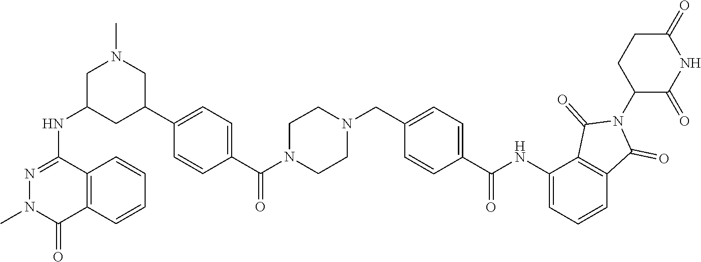 [SMILES: CN1CC(Nc2nn(C)c(=O)c3ccccc23)CC(c2ccc(C(=O)N3CCN(Cc4ccc(C(=O)Nc5cccc6c5C(=O)N(C5CCC(=O)NC5=O)C6=O)cc4)CC3)cc2)C1] |
| | 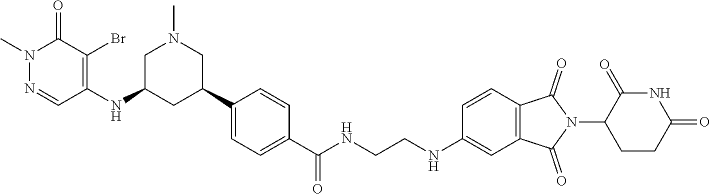 [SMILES: CN1C[C@H](Nc2cnn(C)c(=O)c2Br)C[C@H](c2ccc(C(=O)NCCNc3ccc4c(c3)C(=O)N(C3CCC(=O)NC3=O)C4=O)cc2)C1] |
| | 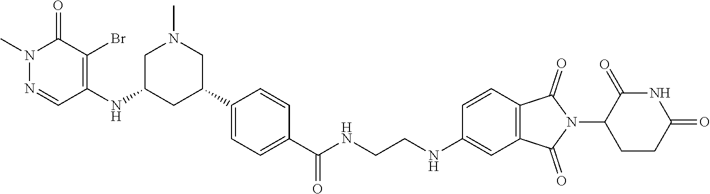 [SMILES: CN1C[C@@H](Nc2cnn(C)c(=O)c2Br)C[C@@H](c2ccc(C(=O)NCCNc3ccc4c(c3)C(=O)N(C3CCC(=O)NC3=O)C4=O)cc2)C1] |
| | 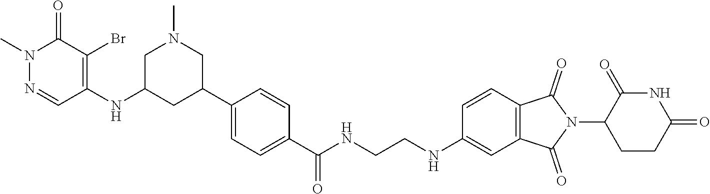 [SMILES: CN1CC(Nc2cnn(C)c(=O)c2Br)CC(c2ccc(C(=O)NCCNc3ccc4c(c3)C(=O)N(C3CCC(=O)NC3=O)C4=O)cc2)C1] |
| | 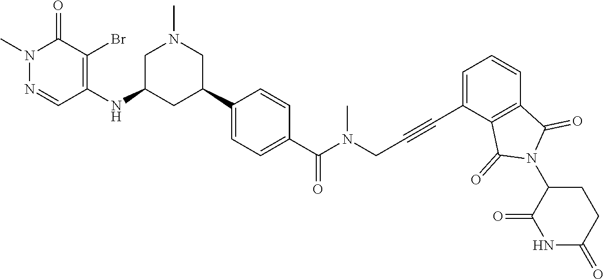 [SMILES: CN1C[C@H](Nc2cnn(C)c(=O)c2Br)C[C@H](c2ccc(C(=O)N(C)CC#Cc3cccc4c3C(=O)N(C3CCC(=O)NC3=O)C4=O)cc2)C1] |
| | 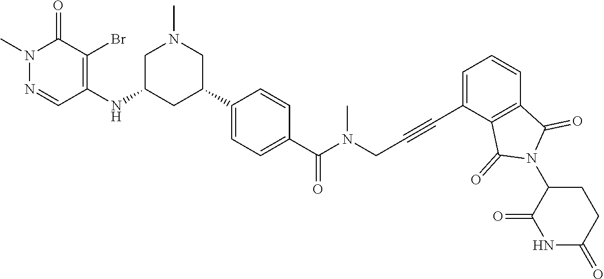 [SMILES: CN1C[C@@H](Nc2cnn(C)c(=O)c2Br)C[C@@H](c2ccc(C(=O)N(C)CC#Cc3cccc4c3C(=O)N(C3CCC(=O)NC3=O)C4=O)cc2)C1] |
| | 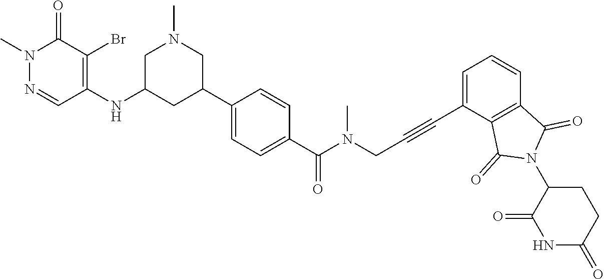 [SMILES: CN1CC(Nc2cnn(C)c(=O)c2Br)CC(c2ccc(C(=O)N(C)CC#Cc3cccc4c3C(=O)N(C3CCC(=O)NC3=O)C4=O)cc2)C1] |
| | 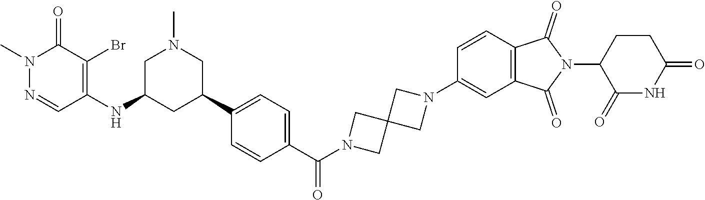 [SMILES: CN1C[C@H](Nc2cnn(C)c(=O)c2Br)C[C@H](c2ccc(C(=O)N3CC4(C3)CN(c3ccc5c(c3)C(=O)N(C3CCC(=O)NC3=O)C5=O)C4)cc2)C1] |
| | 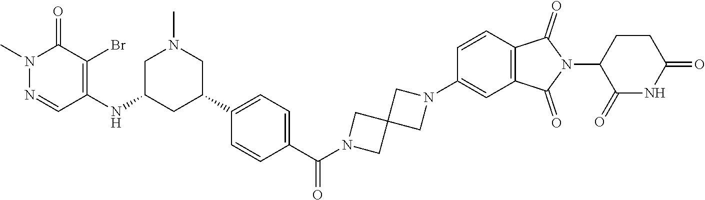 [SMILES: CN1C[C@@H](Nc2cnn(C)c(=O)c2Br)C[C@@H](c2ccc(C(=O)N3CC4(C3)CN(c3ccc5c(c3)C(=O)N(C3CCC(=O)NC3=O)C5=O)C4)cc2)C1] |
| | 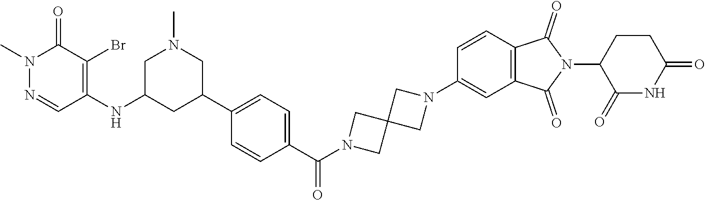 [SMILES: CN1CC(Nc2cnn(C)c(=O)c2Br)CC(c2ccc(C(=O)N3CC4(C3)CN(c3ccc5c(c3)C(=O)N(C3CCC(=O)NC3=O)C5=O)C4)cc2)C1] |
| | 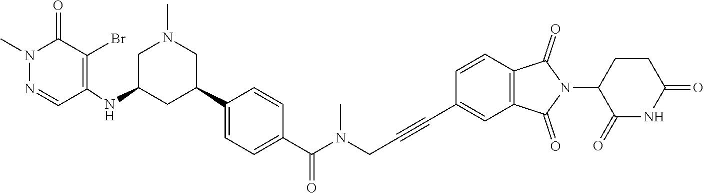 [SMILES: CN1C[C@H](Nc2cnn(C)c(=O)c2Br)C[C@H](c2ccc(C(=O)N(C)CC#Cc3ccc4c(c3)C(=O)N(C3CCC(=O)NC3=O)C4=O)cc2)C1] |
| | 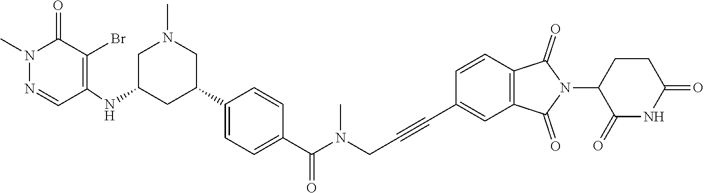 [SMILES: CN1C[C@@H](Nc2cnn(C)c(=O)c2Br)C[C@@H](c2ccc(C(=O)N(C)CC#Cc3ccc4c(c3)C(=O)N(C3CCC(=O)NC3=O)C4=O)cc2)C1] |
| | 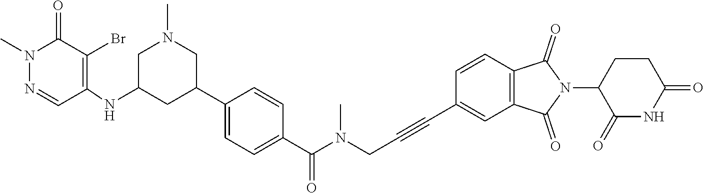 [SMILES: CN1CC(Nc2cnn(C)c(=O)c2Br)CC(c2ccc(C(=O)N(C)CC#Cc3ccc4c(c3)C(=O)N(C3CCC(=O)NC3=O)C4=O)cc2)C1] |
| | 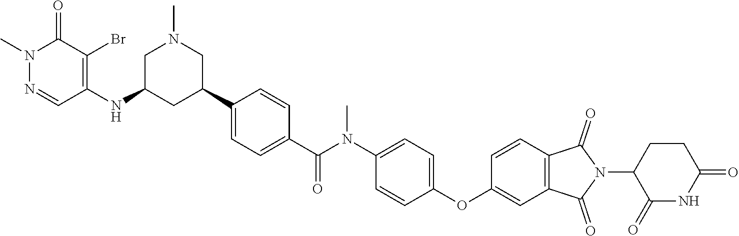 [SMILES: CN1C[C@H](Nc2cnn(C)c(=O)c2Br)C[C@H](c2ccc(C(=O)N(C)c3ccc(Oc4ccc5c(c4)C(=O)N(C4CCC(=O)NC4=O)C5=O)cc3)cc2)C1] |
| | 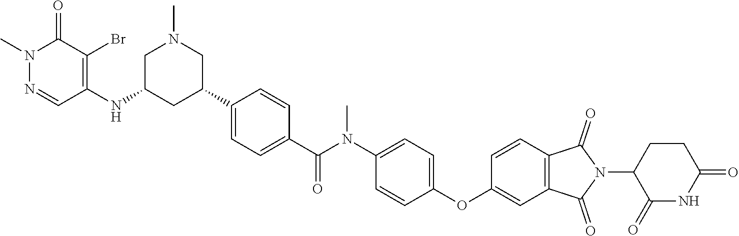 [SMILES: CN1C[C@@H](Nc2cnn(C)c(=O)c2Br)C[C@@H](c2ccc(C(=O)N(C)c3ccc(Oc4ccc5c(c4)C(=O)N(C4CCC(=O)NC4=O)C5=O)cc3)cc2)C1] |
| | 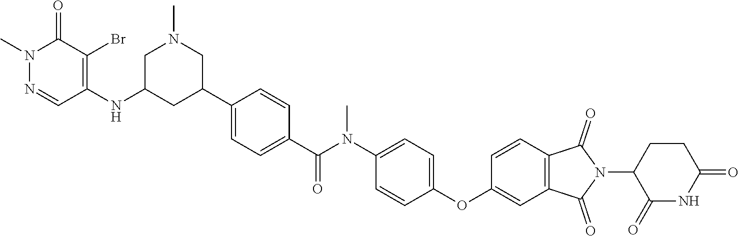 [SMILES: CN1CC(Nc2cnn(C)c(=O)c2Br)CC(c2ccc(C(=O)N(C)c3ccc(Oc4ccc5c(c4)C(=O)N(C4CCC(=O)NC4=O)C5=O)cc3)cc2)C1] |
| | 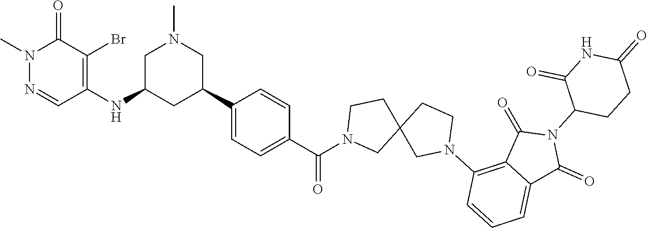 [SMILES: CN1C[C@H](Nc2cnn(C)c(=O)c2Br)C[C@H](c2ccc(C(=O)N3CCC4(CCN(c5cccc6c5C(=O)N(C5CCC(=O)NC5=O)C6=O)C4)C3)cc2)C1] |
| | 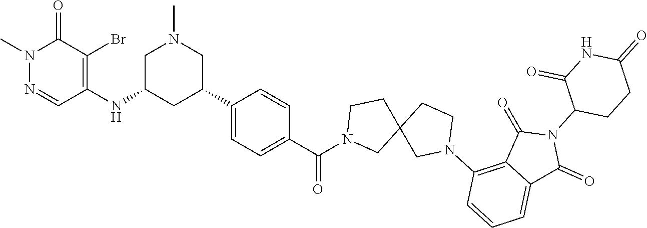 [SMILES: CN1C[C@@H](Nc2cnn(C)c(=O)c2Br)C[C@@H](c2ccc(C(=O)N3CCC4(CCN(c5cccc6c5C(=O)N(C5CCC(=O)NC5=O)C6=O)C4)C3)cc2)C1] |
| | 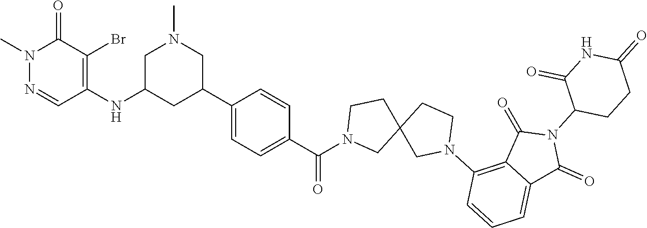 [SMILES: CN1CC(Nc2cnn(C)c(=O)c2Br)CC(c2ccc(C(=O)N3CCC4(CCN(c5cccc6c5C(=O)N(C5CCC(=O)NC5=O)C6=O)C4)C3)cc2)C1] |
| | 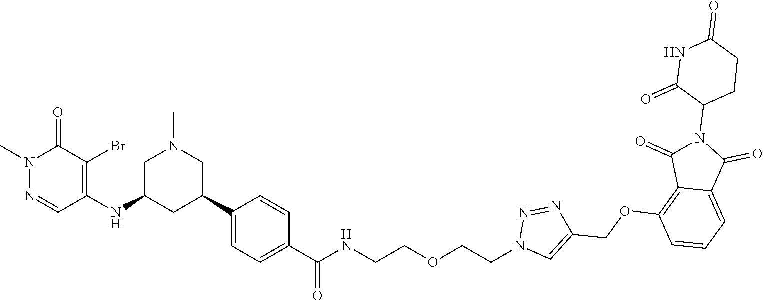 [SMILES: CN1C[C@H](Nc2cnn(C)c(=O)c2Br)C[C@H](c2ccc(C(=O)NCCOCCn3cc(COc4cccc5c4C(=O)N(C4CCC(=O)NC4=O)C5=O)nn3)cc2)C1] |
| | 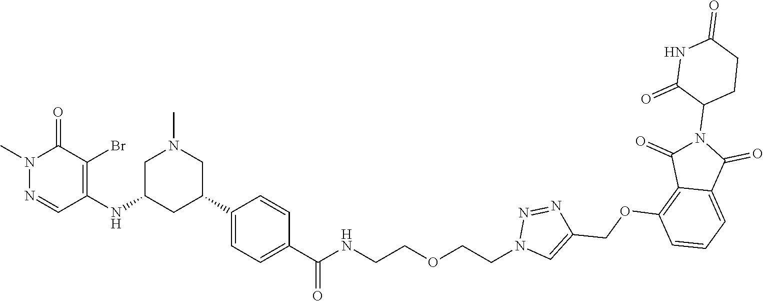 [SMILES: CN1C[C@@H](Nc2cnn(C)c(=O)c2Br)C[C@@H](c2ccc(C(=O)NCCOCCn3cc(COc4cccc5c4C(=O)N(C4CCC(=O)NC4=O)C5=O)nn3)cc2)C1] |
| | 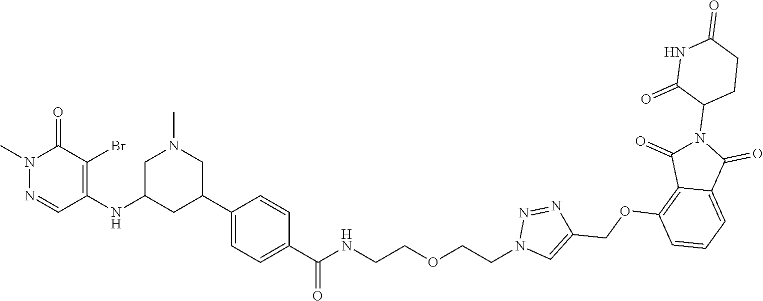 [SMILES: CN1CC(Nc2cnn(C)c(=O)c2Br)CC(c2ccc(C(=O)NCCOCCn3cc(COc4cccc5c4C(=O)N(C4CCC(=O)NC4=O)C5=O)nn3)cc2)C1] |
| | 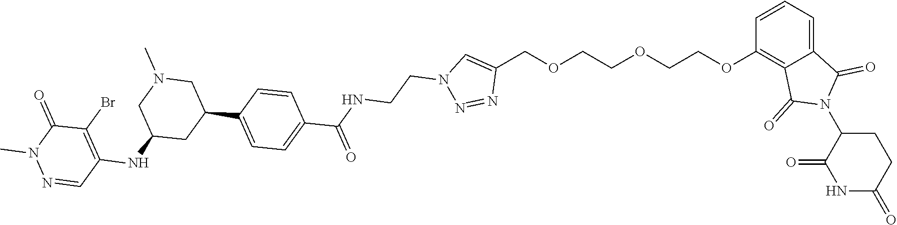 [SMILES: CN1C[C@H](Nc2cnn(C)c(=O)c2Br)C[C@H](c2ccc(C(=O)NCCn3cc(COCCOCCOc4cccc5c4C(=O)N(C4CCC(=O)NC4=O)C5=O)nn3)cc2)C1] |
| | 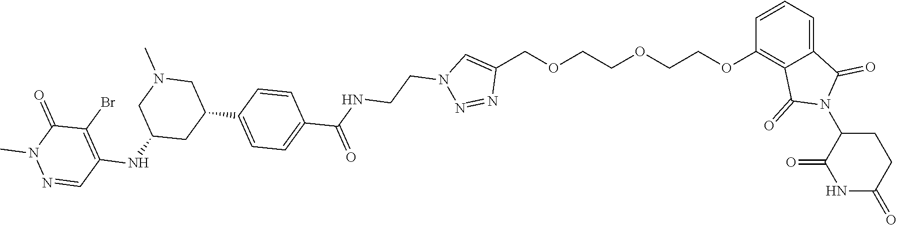 [SMILES: CN1C[C@@H](Nc2cnn(C)c(=O)c2Br)C[C@@H](c2ccc(C(=O)NCCn3cc(COCCOCCOc4cccc5c4C(=O)N(C4CCC(=O)NC4=O)C5=O)nn3)cc2)C1] |
| | 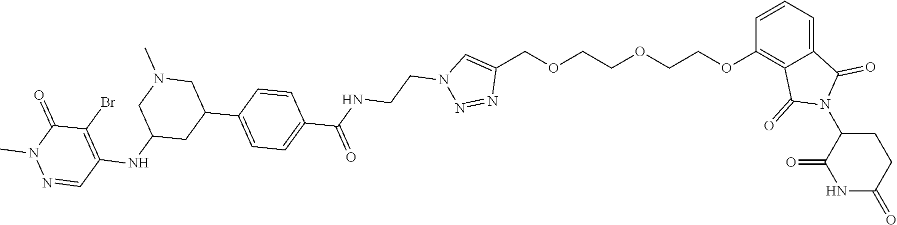 [SMILES: CN1CC(Nc2cnn(C)c(=O)c2Br)CC(c2ccc(C(=O)NCCn3cc(COCCOCCOc4cccc5c4C(=O)N(C4CCC(=O)NC4=O)C5=O)nn3)cc2)C1] |
| | 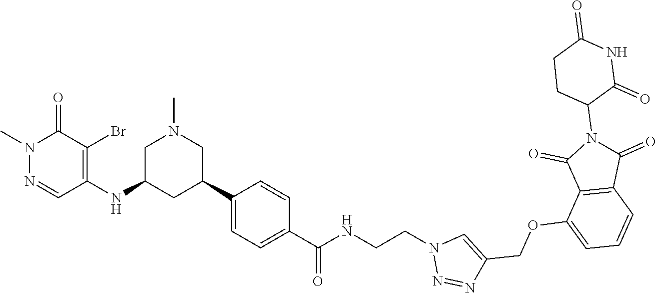 [SMILES: CN1C[C@H](Nc2cnn(C)c(=O)c2Br)C[C@H](c2ccc(C(=O)NCCn3cc(COc4cccc5c4C(=O)N(C4CCC(=O)NC4=O)C5=O)nn3)cc2)C1] |
| | 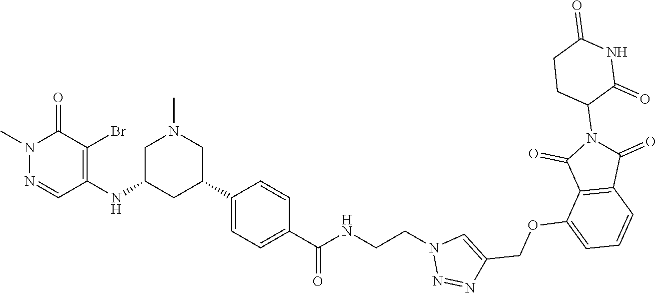 [SMILES: CN1C[C@@H](Nc2cnn(C)c(=O)c2Br)C[C@@H](c2ccc(C(=O)NCCn3cc(COc4cccc5c4C(=O)N(C4CCC(=O)NC4=O)C5=O)nn3)cc2)C1] |
| | 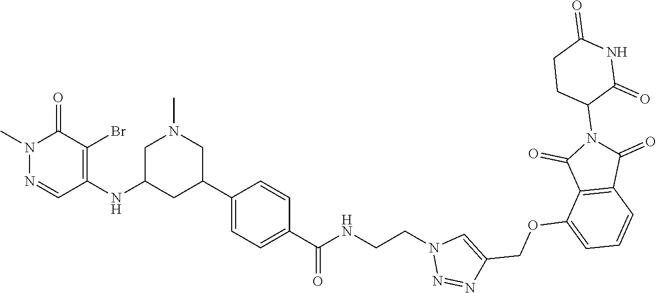 [SMILES: CN1CC(Nc2cnn(C)c(=O)c2Br)CC(c2ccc(C(=O)NCCn3cc(COc4cccc5c4C(=O)N(C4CCC(=O)NC4=O)C5=O)nn3)cc2)C1] |
| | 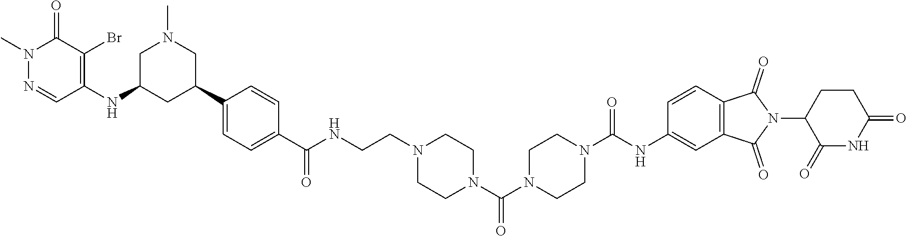 [SMILES: CN1C[C@H](Nc2cnn(C)c(=O)c2Br)C[C@H](c2ccc(C(=O)NCCN3CCN(C(=O)N4CCN(C(=O)Nc5ccc6c(c5)C(=O)N(C5CCC(=O)NC5=O)C6=O)CC4)CC3)cc2)C1] |
| | 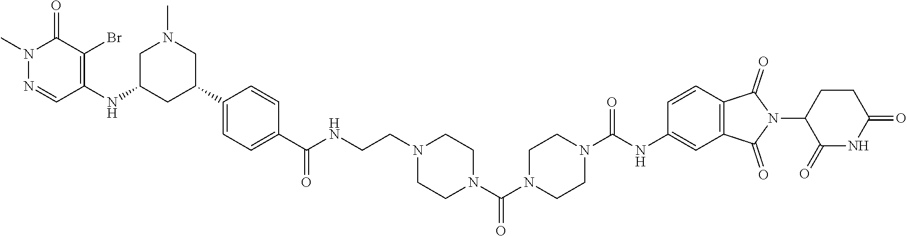 [SMILES: CN1C[C@@H](Nc2cnn(C)c(=O)c2Br)C[C@@H](c2ccc(C(=O)NCCN3CCN(C(=O)N4CCN(C(=O)Nc5ccc6c(c5)C(=O)N(C5CCC(=O)NC5=O)C6=O)CC4)CC3)cc2)C1] |
| | 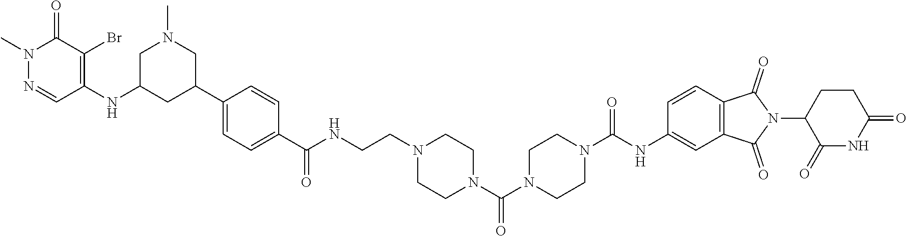 [SMILES: CN1CC(Nc2cnn(C)c(=O)c2Br)CC(c2ccc(C(=O)NCCN3CCN(C(=O)N4CCN(C(=O)Nc5ccc6c(c5)C(=O)N(C5CCC(=O)NC5=O)C6=O)CC4)CC3)cc2)C1] |
| | 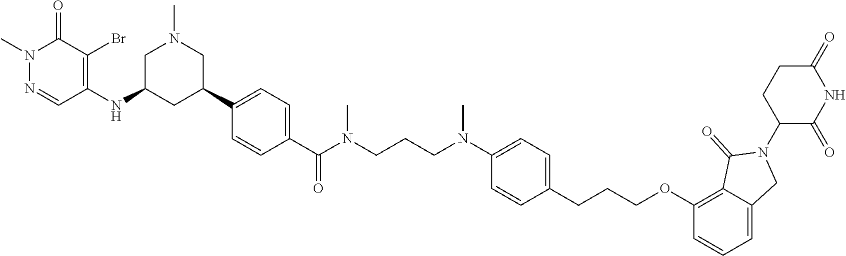 [SMILES: CN1C[C@H](Nc2cnn(C)c(=O)c2Br)C[C@H](c2ccc(C(=O)N(C)CCCN(C)c3ccc(CCCOc4cccc5c4C(=O)N(C4CCC(=O)NC4=O)C5)cc3)cc2)C1] |
| | 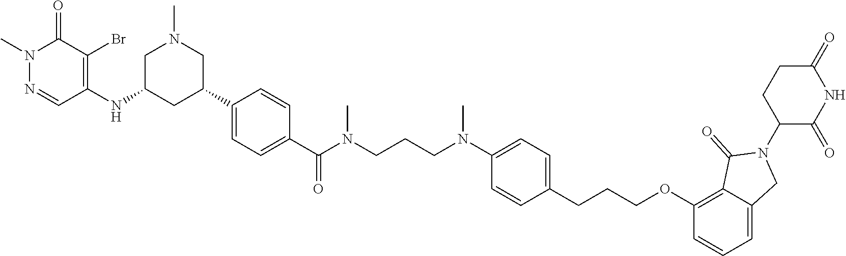 [SMILES: CN1C[C@@H](Nc2cnn(C)c(=O)c2Br)C[C@@H](c2ccc(C(=O)N(C)CCCN(C)c3ccc(CCCOc4cccc5c4C(=O)N(C4CCC(=O)NC4=O)C5)cc3)cc2)C1] |
| | 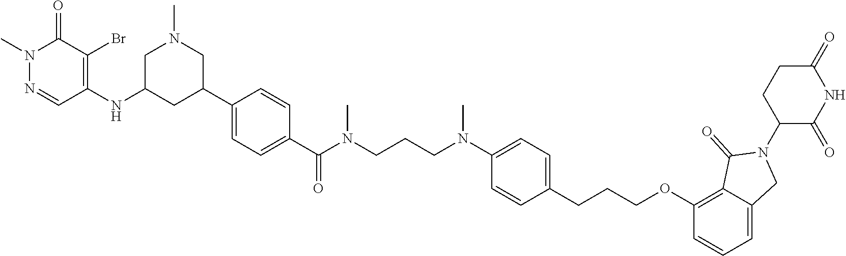 [SMILES: CN1CC(Nc2cnn(C)c(=O)c2Br)CC(c2ccc(C(=O)N(C)CCCN(C)c3ccc(CCCOc4cccc5c4C(=O)N(C4CCC(=O)NC4=O)C5)cc3)cc2)C1] |
| | 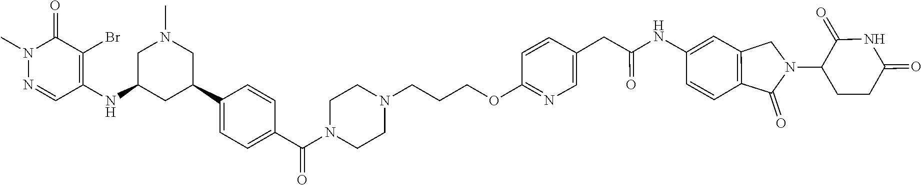 [SMILES: CN1C[C@H](Nc2cnn(C)c(=O)c2Br)C[C@H](c2ccc(C(=O)N3CCN(CCCOc4ccc(CC(=O)Nc5ccc6c(c5)CN(C5CCC(=O)NC5=O)C6=O)cn4)CC3)cc2)C1] |
| | 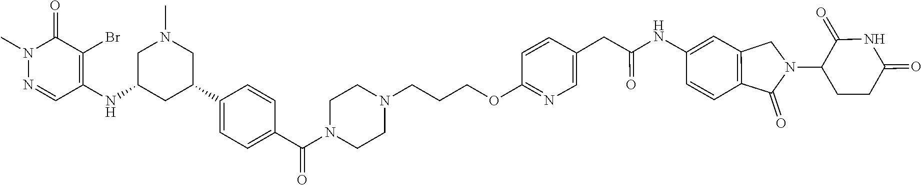 [SMILES: CN1C[C@@H](Nc2cnn(C)c(=O)c2Br)C[C@@H](c2ccc(C(=O)N3CCN(CCCOc4ccc(CC(=O)Nc5ccc6c(c5)CN(C5CCC(=O)NC5=O)C6=O)cn4)CC3)cc2)C1] |
| | 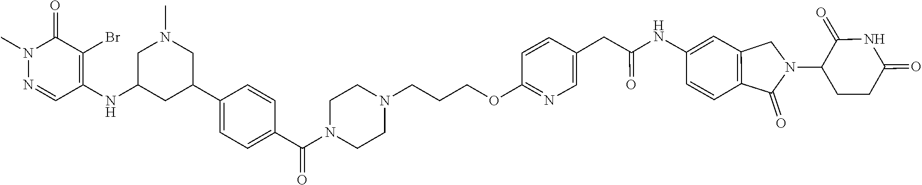 [SMILES: CN1CC(Nc2cnn(C)c(=O)c2Br)CC(c2ccc(C(=O)N3CCN(CCCOc4ccc(CC(=O)Nc5ccc6c(c5)CN(C5CCC(=O)NC5=O)C6=O)cn4)CC3)cc2)C1] |
| | 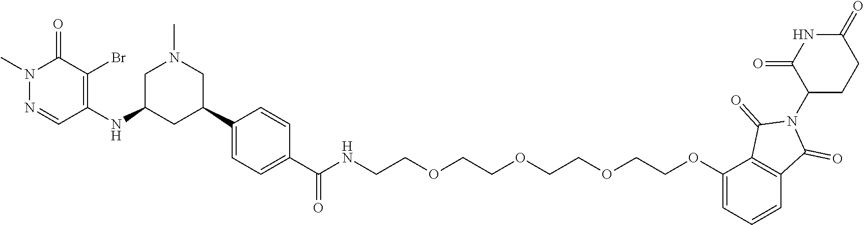 [SMILES: CN1C[C@H](Nc2cnn(C)c(=O)c2Br)C[C@H](c2ccc(C(=O)NCCOCCOCCOCCOc3cccc4c3C(=O)N(C3CCC(=O)NC3=O)C4=O)cc2)C1] |
| | 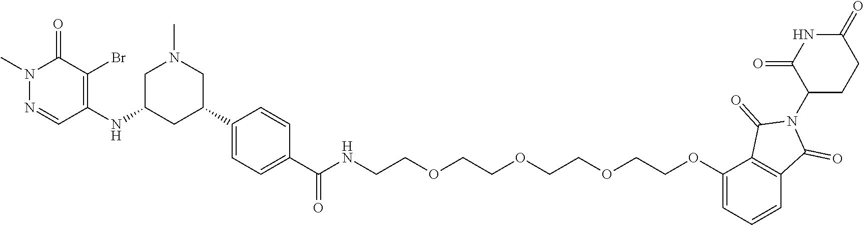 [SMILES: CN1C[C@@H](Nc2cnn(C)c(=O)c2Br)C[C@@H](c2ccc(C(=O)NCCOCCOCCOCCOc3cccc4c3C(=O)N(C3CCC(=O)NC3=O)C4=O)cc2)C1] |
| | 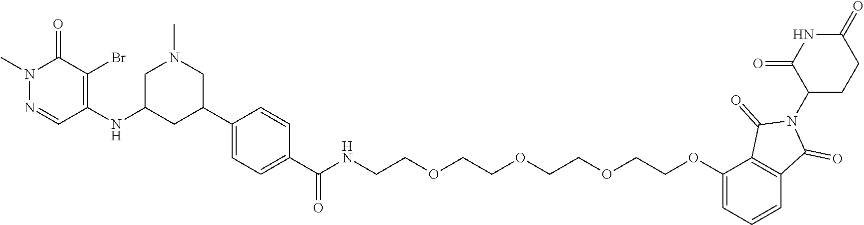 [SMILES: CN1CC(Nc2cnn(C)c(=O)c2Br)CC(c2ccc(C(=O)NCCOCCOCCOCCOc3cccc4c3C(=O)N(C3CCC(=O)NC3=O)C4=O)cc2)C1] |
| | 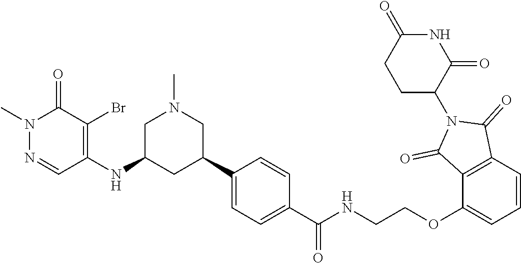 [SMILES: CN1C[C@H](Nc2cnn(C)c(=O)c2Br)C[C@H](c2ccc(C(=O)NCCOc3cccc4c3C(=O)N(C3CCC(=O)NC3=O)C4=O)cc2)C1] |
| | 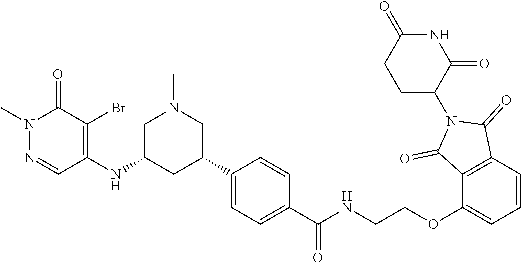 [SMILES: CN1C[C@@H](Nc2cnn(C)c(=O)c2Br)C[C@@H](c2ccc(C(=O)NCCOc3cccc4c3C(=O)N(C3CCC(=O)NC3=O)C4=O)cc2)C1] |
| | 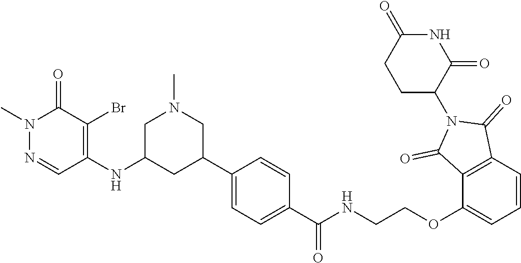 [SMILES: CN1CC(Nc2cnn(C)c(=O)c2Br)CC(c2ccc(C(=O)NCCOc3cccc4c3C(=O)N(C3CCC(=O)NC3=O)C4=O)cc2)C1] |
| | 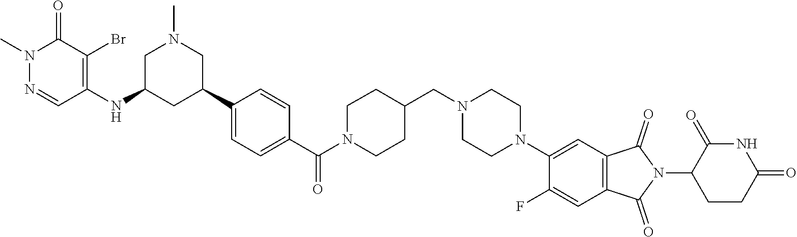 [SMILES: CN1C[C@H](Nc2cnn(C)c(=O)c2Br)C[C@H](c2ccc(C(=O)N3CCC(CN4CCN(c5cc6c(cc5F)C(=O)N(C5CCC(=O)NC5=O)C6=O)CC4)CC3)cc2)C1] |
| | 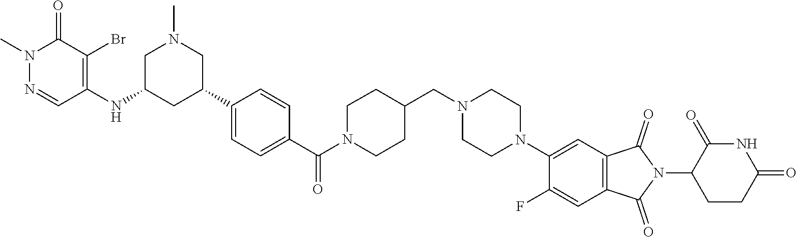 [SMILES: CN1C[C@@H](Nc2cnn(C)c(=O)c2Br)C[C@@H](c2ccc(C(=O)N3CCC(CN4CCN(c5cc6c(cc5F)C(=O)N(C5CCC(=O)NC5=O)C6=O)CC4)CC3)cc2)C1] |
| | 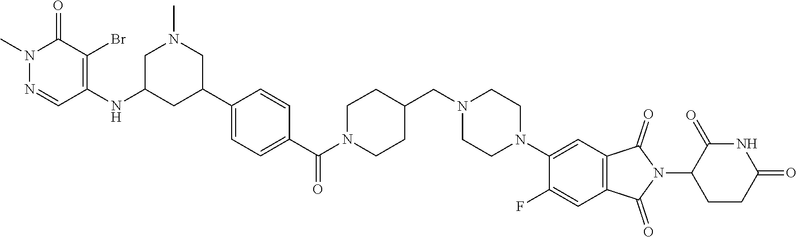 [SMILES: CN1CC(Nc2cnn(C)c(=O)c2Br)CC(c2ccc(C(=O)N3CCC(CN4CCN(c5cc6c(cc5F)C(=O)N(C5CCC(=O)NC5=O)C6=O)CC4)CC3)cc2)C1] |
| | 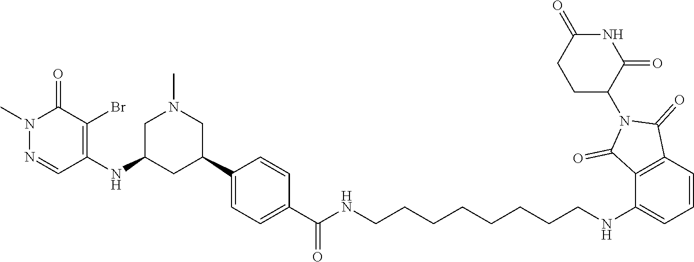 [SMILES: CN1C[C@H](Nc2cnn(C)c(=O)c2Br)C[C@H](c2ccc(C(=O)NCCCCCCCCNc3cccc4c3C(=O)N(C3CCC(=O)NC3=O)C4=O)cc2)C1] |
| | 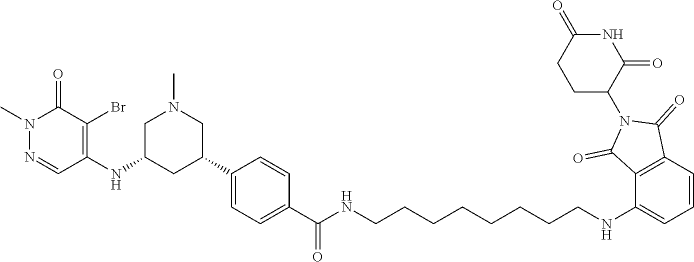 [SMILES: CN1C[C@@H](Nc2cnn(C)c(=O)c2Br)C[C@@H](c2ccc(C(=O)NCCCCCCCCNc3cccc4c3C(=O)N(C3CCC(=O)NC3=O)C4=O)cc2)C1] |
| | 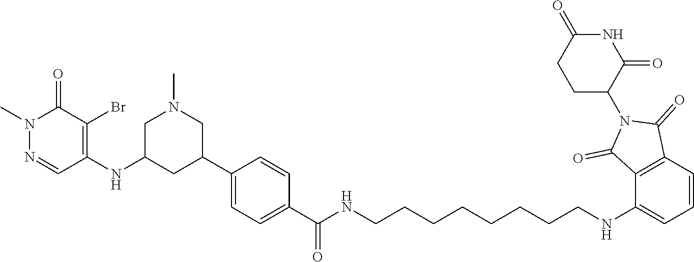 [SMILES: CN1CC(Nc2cnn(C)c(=O)c2Br)CC(c2ccc(C(=O)NCCCCCCCCNc3cccc4c3C(=O)N(C3CCC(=O)NC3=O)C4=O)cc2)C1] |
| | 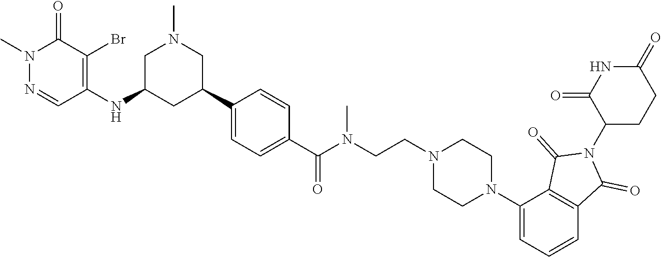 [SMILES: CN1C[C@H](Nc2cnn(C)c(=O)c2Br)C[C@H](c2ccc(C(=O)N(C)CCN3CCN(c4cccc5c4C(=O)N(C4CCC(=O)NC4=O)C5=O)CC3)cc2)C1] |
| | 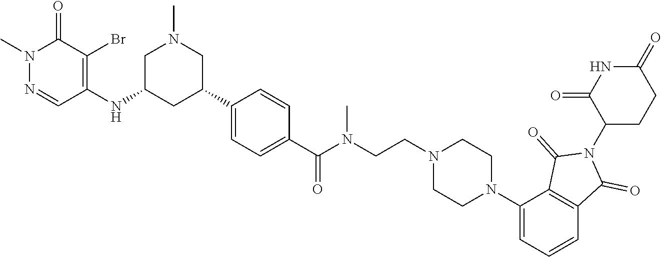 [SMILES: CN1C[C@@H](Nc2cnn(C)c(=O)c2Br)C[C@@H](c2ccc(C(=O)N(C)CCN3CCN(c4cccc5c4C(=O)N(C4CCC(=O)NC4=O)C5=O)CC3)cc2)C1] |
| | 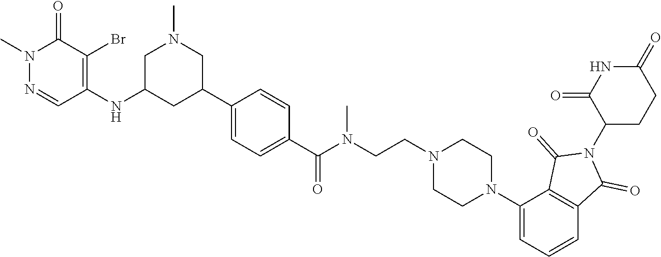 [SMILES: CN1CC(Nc2cnn(C)c(=O)c2Br)CC(c2ccc(C(=O)N(C)CCN3CCN(c4cccc5c4C(=O)N(C4CCC(=O)NC4=O)C5=O)CC3)cc2)C1] |
| | 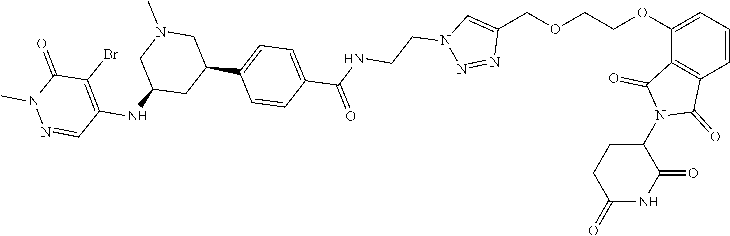 [SMILES: CN1C[C@H](Nc2cnn(C)c(=O)c2Br)C[C@H](c2ccc(C(=O)NCCn3cc(COCCOc4cccc5c4C(=O)N(C4CCC(=O)NC4=O)C5=O)nn3)cc2)C1] |
| | 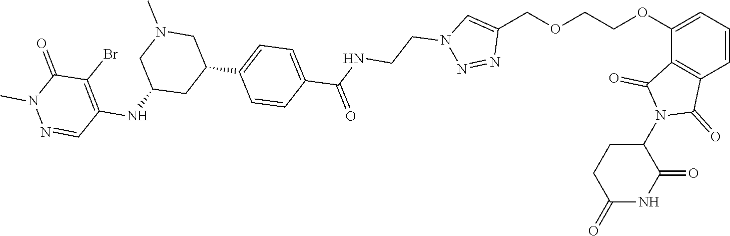 [SMILES: CN1C[C@@H](Nc2cnn(C)c(=O)c2Br)C[C@@H](c2ccc(C(=O)NCCn3cc(COCCOc4cccc5c4C(=O)N(C4CCC(=O)NC4=O)C5=O)nn3)cc2)C1] |
| | 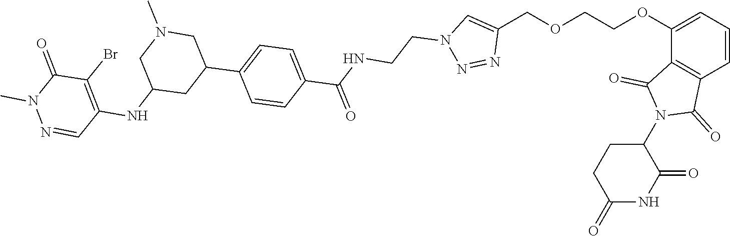 [SMILES: CN1CC(Nc2cnn(C)c(=O)c2Br)CC(c2ccc(C(=O)NCCn3cc(COCCOc4cccc5c4C(=O)N(C4CCC(=O)NC4=O)C5=O)nn3)cc2)C1] |
| | 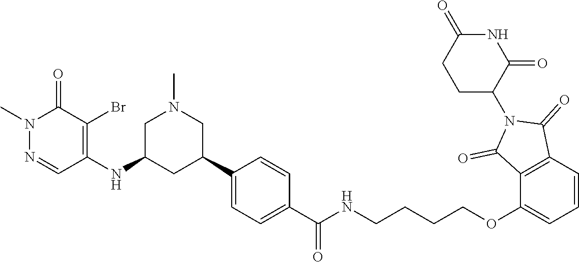 [SMILES: CN1C[C@H](Nc2cnn(C)c(=O)c2Br)C[C@H](c2ccc(C(=O)NCCCCOc3cccc4c3C(=O)N(C3CCC(=O)NC3=O)C4=O)cc2)C1] |
| | 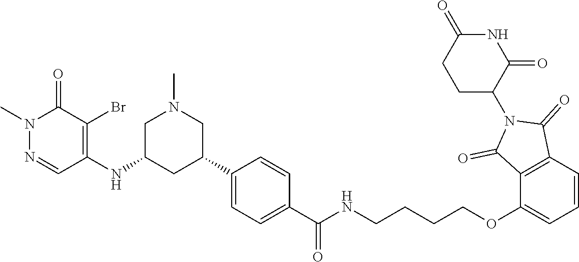 [SMILES: CN1C[C@@H](Nc2cnn(C)c(=O)c2Br)C[C@@H](c2ccc(C(=O)NCCCCOc3cccc4c3C(=O)N(C3CCC(=O)NC3=O)C4=O)cc2)C1] |
| | 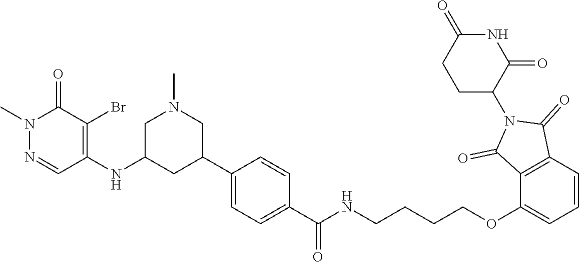 [SMILES: CN1CC(Nc2cnn(C)c(=O)c2Br)CC(c2ccc(C(=O)NCCCCOc3cccc4c3C(=O)N(C3CCC(=O)NC3=O)C4=O)cc2)C1] |
| | 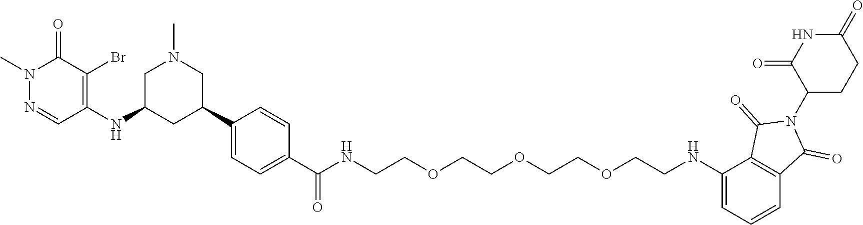 [SMILES: CN1C[C@H](Nc2cnn(C)c(=O)c2Br)C[C@H](c2ccc(C(=O)NCCOCCOCCOCCNc3cccc4c3C(=O)N(C3CCC(=O)NC3=O)C4=O)cc2)C1] |
| | 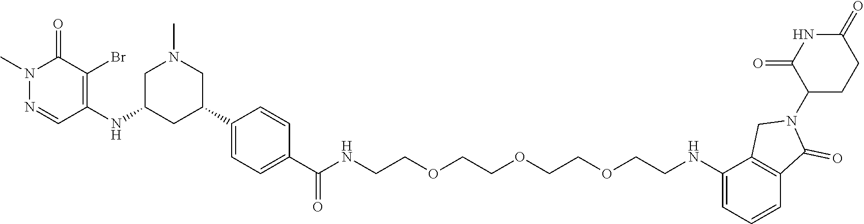 [SMILES: CN1C[C@@H](Nc2cnn(C)c(=O)c2Br)C[C@@H](c2ccc(C(=O)NCCOCCOCCOCCNc3cccc4c3CN(C3CCC(=O)NC3=O)C4=O)cc2)C1] |
| | 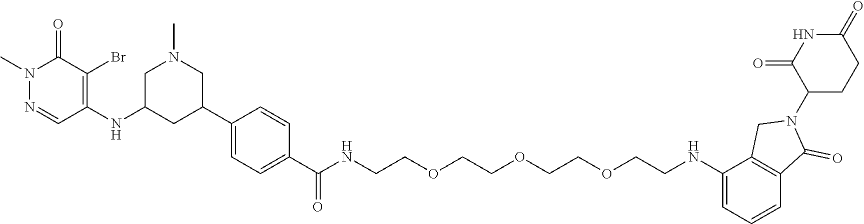 [SMILES: CN1CC(Nc2cnn(C)c(=O)c2Br)CC(c2ccc(C(=O)NCCOCCOCCOCCNc3cccc4c3CN(C3CCC(=O)NC3=O)C4=O)cc2)C1] |
| | 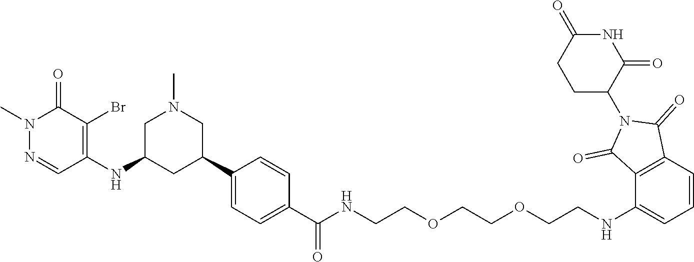 [SMILES: CN1C[C@H](Nc2cnn(C)c(=O)c2Br)C[C@H](c2ccc(C(=O)NCCOCCOCCNc3cccc4c3C(=O)N(C3CCC(=O)NC3=O)C4=O)cc2)C1] |
| | 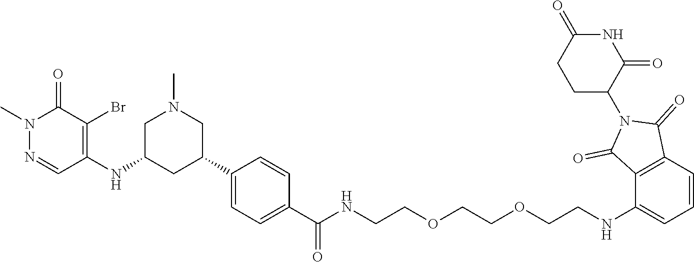 [SMILES: CN1C[C@@H](Nc2cnn(C)c(=O)c2Br)C[C@@H](c2ccc(C(=O)NCCOCCOCCNc3cccc4c3C(=O)N(C3CCC(=O)NC3=O)C4=O)cc2)C1] |
| | 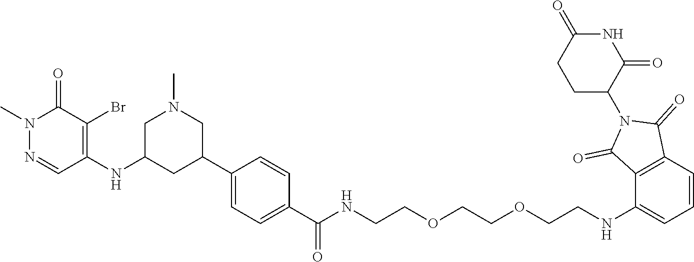 [SMILES: CN1CC(Nc2cnn(C)c(=O)c2Br)CC(c2ccc(C(=O)NCCOCCOCCNc3cccc4c3C(=O)N(C3CCC(=O)NC3=O)C4=O)cc2)C1] |
| | 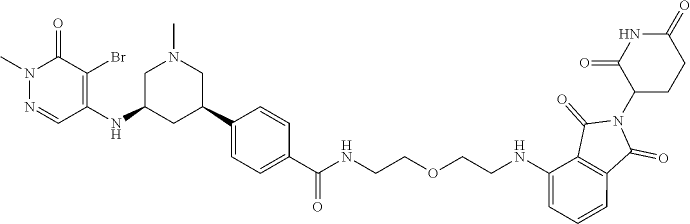 [SMILES: CN1C[C@H](Nc2cnn(C)c(=O)c2Br)C[C@H](c2ccc(C(=O)NCCOCCNc3cccc4c3C(=O)N(C3CCC(=O)NC3=O)C4=O)cc2)C1] |
| | 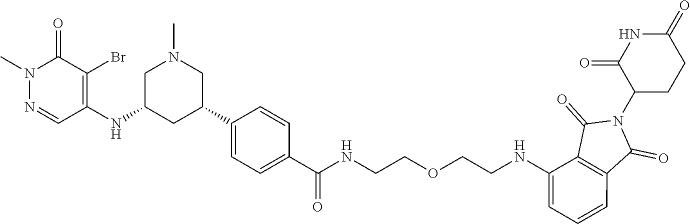 [SMILES: CN1C[C@@H](Nc2cnn(C)c(=O)c2Br)C[C@@H](c2ccc(C(=O)NCCOCCNc3cccc4c3C(=O)N(C3CCC(=O)NC3=O)C4=O)cc2)C1] |
| | 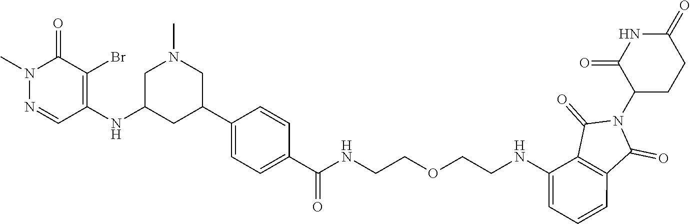 [SMILES: CN1CC(Nc2cnn(C)c(=O)c2Br)CC(c2ccc(C(=O)NCCOCCNc3cccc4c3C(=O)N(C3CCC(=O)NC3=O)C4=O)cc2)C1] |
| | 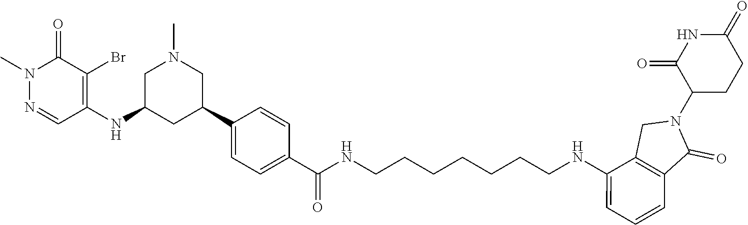 [SMILES: CN1C[C@H](Nc2cnn(C)c(=O)c2Br)C[C@H](c2ccc(C(=O)NCCCCCCCNc3cccc4c3CN(C3CCC(=O)NC3=O)C4=O)cc2)C1] |
| | 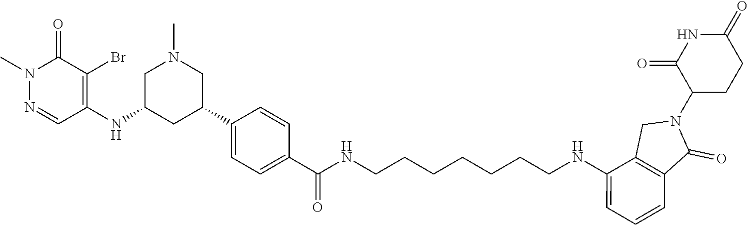 [SMILES: CN1C[C@@H](Nc2cnn(C)c(=O)c2Br)C[C@@H](c2ccc(C(=O)NCCCCCCCNc3cccc4c3CN(C3CCC(=O)NC3=O)C4=O)cc2)C1] |
| | 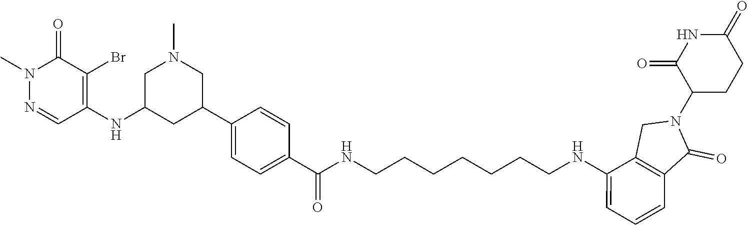 [SMILES: CN1CC(Nc2cnn(C)c(=O)c2Br)CC(c2ccc(C(=O)NCCCCCCCNc3cccc4c3CN(C3CCC(=O)NC3=O)C4=O)cc2)C1] |
| | 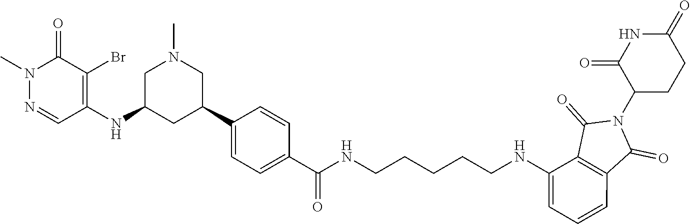 [SMILES: CN1C[C@H](Nc2cnn(C)c(=O)c2Br)C[C@H](c2ccc(C(=O)NCCCCCNc3cccc4c3C(=O)N(C3CCC(=O)NC3=O)C4=O)cc2)C1] |
| | 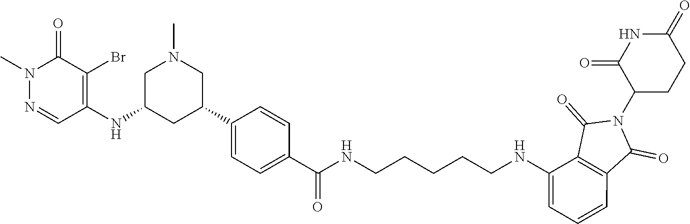 [SMILES: CN1C[C@@H](Nc2cnn(C)c(=O)c2Br)C[C@@H](c2ccc(C(=O)NCCCCCNc3cccc4c3C(=O)N(C3CCC(=O)NC3=O)C4=O)cc2)C1] |
| | 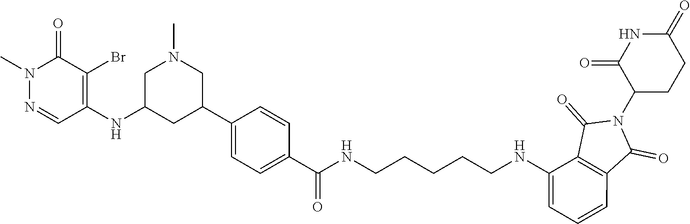 [SMILES: CN1CC(Nc2cnn(C)c(=O)c2Br)CC(c2ccc(C(=O)NCCCCCNc3cccc4c3C(=O)N(C3CCC(=O)NC3=O)C4=O)cc2)C1] |
| | 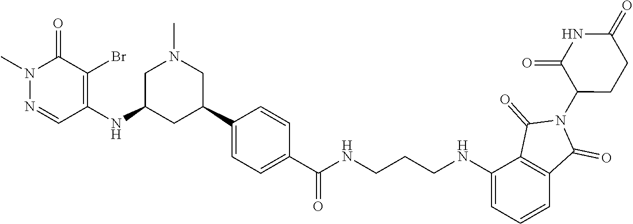 [SMILES: CN1C[C@H](Nc2cnn(C)c(=O)c2Br)C[C@H](c2ccc(C(=O)NCCCNc3cccc4c3C(=O)N(C3CCC(=O)NC3=O)C4=O)cc2)C1] |
| | 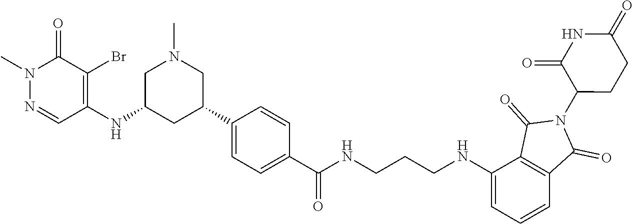 [SMILES: CN1C[C@@H](Nc2cnn(C)c(=O)c2Br)C[C@@H](c2ccc(C(=O)NCCCNc3cccc4c3C(=O)N(C3CCC(=O)NC3=O)C4=O)cc2)C1] |
| | 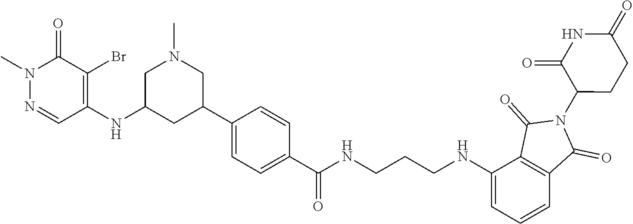 [SMILES: CN1CC(Nc2cnn(C)c(=O)c2Br)CC(c2ccc(C(=O)NCCCNc3cccc4c3C(=O)N(C3CCC(=O)NC3=O)C4=O)cc2)C1] |
| | 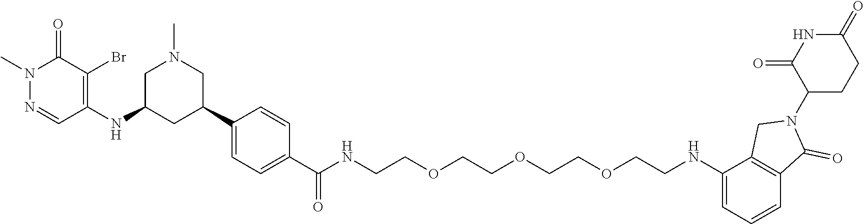 [SMILES: CN1C[C@H](Nc2cnn(C)c(=O)c2Br)C[C@H](c2ccc(C(=O)NCCOCCOCCOCCNc3cccc4c3CN(C3CCC(=O)NC3=O)C4=O)cc2)C1] |
| | 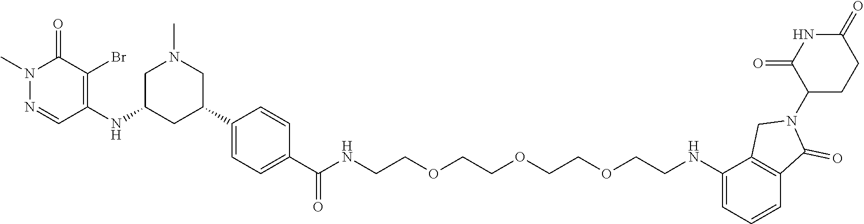 [SMILES: CN1C[C@@H](Nc2cnn(C)c(=O)c2Br)C[C@@H](c2ccc(C(=O)NCCOCCOCCOCCNc3cccc4c3CN(C3CCC(=O)NC3=O)C4=O)cc2)C1] |
| | 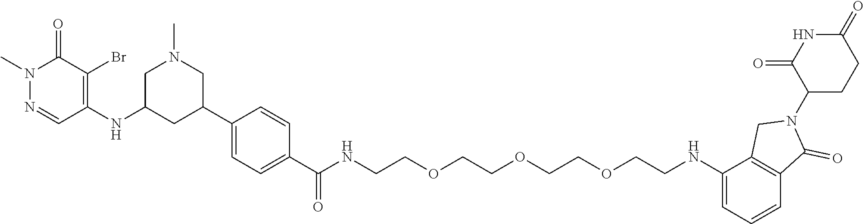 [SMILES: CN1CC(Nc2cnn(C)c(=O)c2Br)CC(c2ccc(C(=O)NCCOCCOCCOCCNc3cccc4c3CN(C3CCC(=O)NC3=O)C4=O)cc2)C1] |
| | 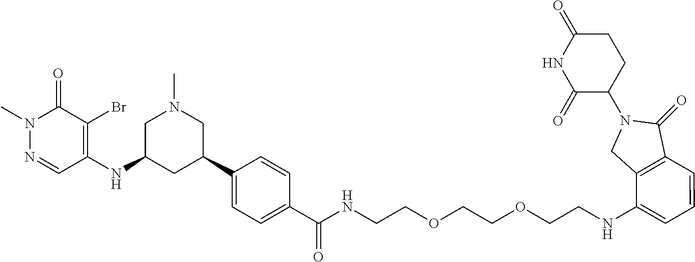 [SMILES: CN1C[C@H](Nc2cnn(C)c(=O)c2Br)C[C@H](c2ccc(C(=O)NCCOCCOCCNc3cccc4c3CN(C3CCC(=O)NC3=O)C4=O)cc2)C1] |
| | 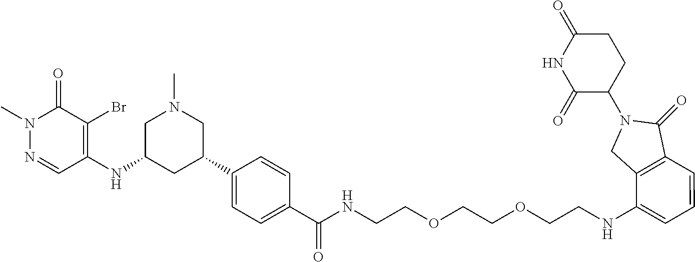 [SMILES: CN1C[C@@H](Nc2cnn(C)c(=O)c2Br)C[C@@H](c2ccc(C(=O)NCCOCCOCCNc3cccc4c3CN(C3CCC(=O)NC3=O)C4=O)cc2)C1] |
| | 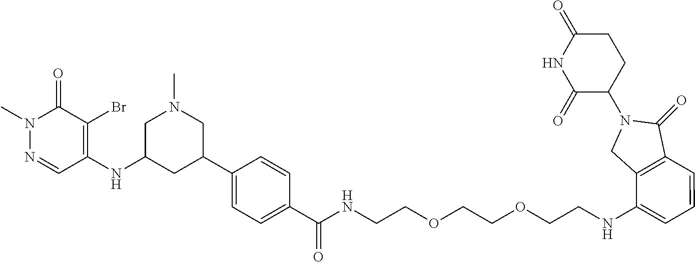 [SMILES: CN1CC(Nc2cnn(C)c(=O)c2Br)CC(c2ccc(C(=O)NCCOCCOCCNc3cccc4c3CN(C3CCC(=O)NC3=O)C4=O)cc2)C1] |
| 2-15 | 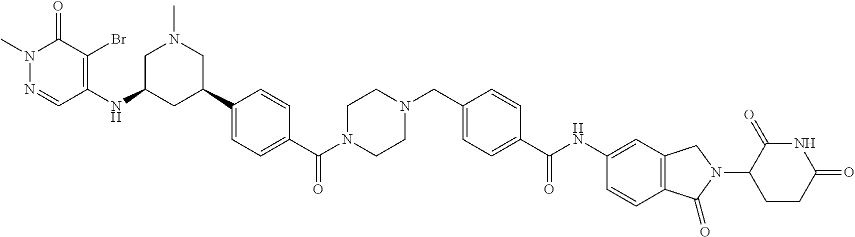 [SMILES: CN1C[C@H](Nc2cnn(C)c(=O)c2Br)C[C@H](c2ccc(C(=O)N3CCN(Cc4ccc(C(=O)Nc5ccc6c(c5)CN(C5CCC(=O)NC5=O)C6=O)cc4)CC3)cc2)C1] |
| | 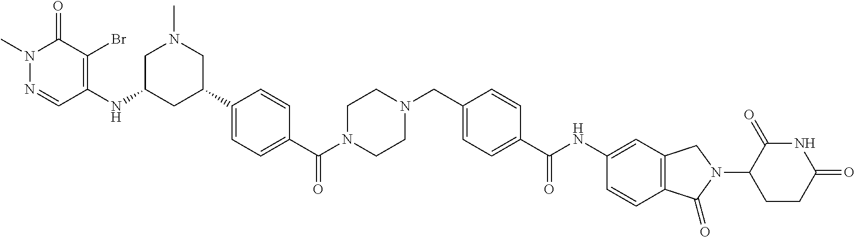 [SMILES: CN1C[C@@H](Nc2cnn(C)c(=O)c2Br)C[C@@H](c2ccc(C(=O)N3CCN(Cc4ccc(C(=O)Nc5ccc6c(c5)CN(C5CCC(=O)NC5=O)C6=O)cc4)CC3)cc2)C1] |
| | 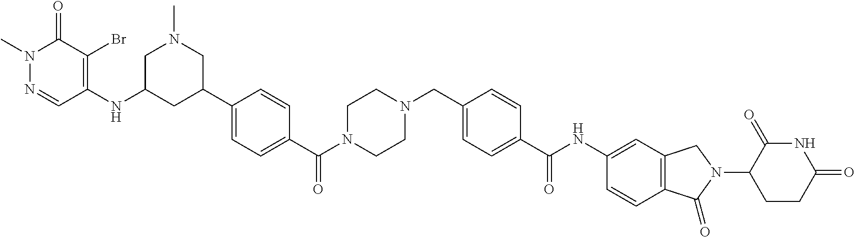 [SMILES: CN1CC(Nc2cnn(C)c(=O)c2Br)CC(c2ccc(C(=O)N3CCN(Cc4ccc(C(=O)Nc5ccc6c(c5)CN(C5CCC(=O)NC5=O)C6=O)cc4)CC3)cc2)C1] |
| | 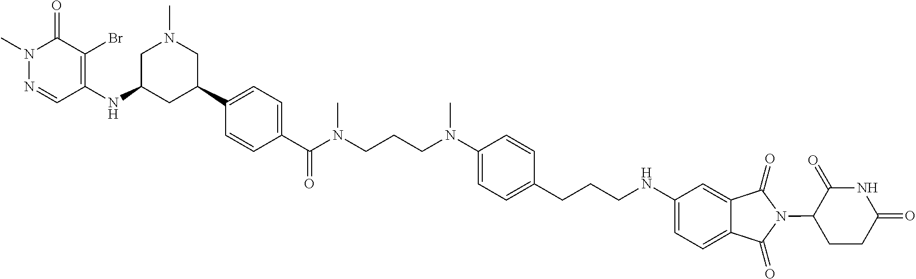 [SMILES: CN1C[C@H](Nc2cnn(C)c(=O)c2Br)C[C@H](c2ccc(C(=O)N(C)CCCN(C)c3ccc(CCCNc4ccc5c(c4)C(=O)N(C4CCC(=O)NC4=O)C5=O)cc3)cc2)C1] |
| | 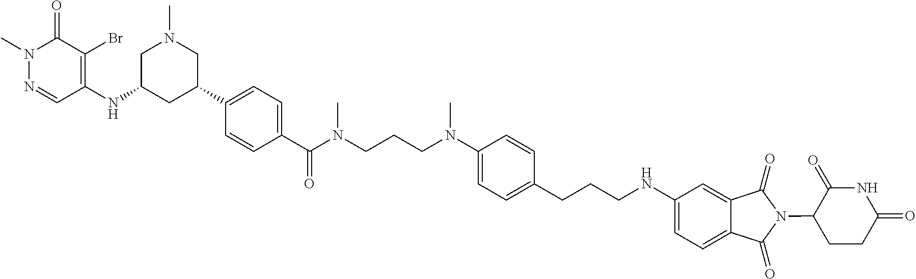 [SMILES: CN1C[C@@H](Nc2cnn(C)c(=O)c2Br)C[C@@H](c2ccc(C(=O)N(C)CCCN(C)c3ccc(CCCNc4ccc5c(c4)C(=O)N(C4CCC(=O)NC4=O)C5=O)cc3)cc2)C1] |
| | 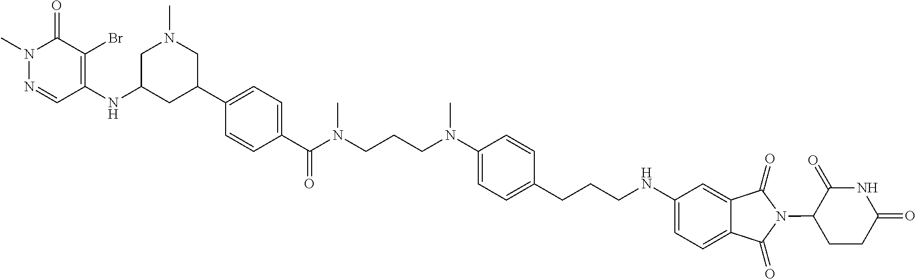 [SMILES: CN1CC(Nc2cnn(C)c(=O)c2Br)CC(c2ccc(C(=O)N(C)CCCN(C)c3ccc(CCCNc4ccc5c(c4)C(=O)N(C4CCC(=O)NC4=O)C5=O)cc3)cc2)C1] |
| | 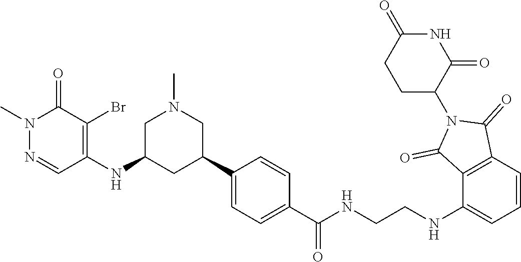 [SMILES: CN1C[C@H](Nc2cnn(C)c(=O)c2Br)C[C@H](c2ccc(C(=O)NCCNc3cccc4c3C(=O)N(C3CCC(=O)NC3=O)C4=O)cc2)C1] |
| | 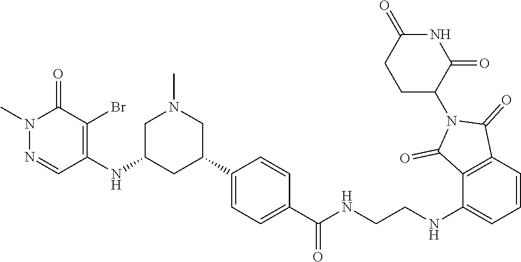 [SMILES: CN1C[C@@H](Nc2cnn(C)c(=O)c2Br)C[C@@H](c2ccc(C(=O)NCCNc3cccc4c3C(=O)N(C3CCC(=O)NC3=O)C4=O)cc2)C1] |
| | 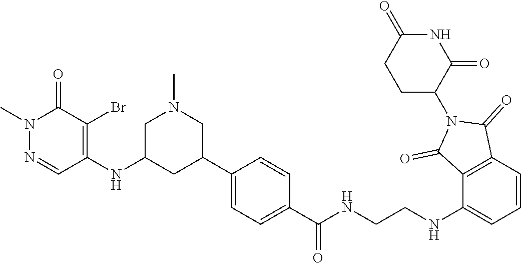 [SMILES: CN1CC(Nc2cnn(C)c(=O)c2Br)CC(c2ccc(C(=O)NCCNc3cccc4c3C(=O)N(C3CCC(=O)NC3=O)C4=O)cc2)C1] |
| | 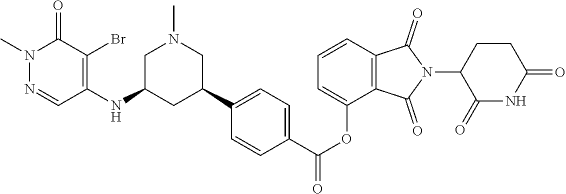 [SMILES: CN1C[C@H](Nc2cnn(C)c(=O)c2Br)C[C@H](c2ccc(C(=O)Oc3cccc4c3C(=O)N(C3CCC(=O)NC3=O)C4=O)cc2)C1] |
| | 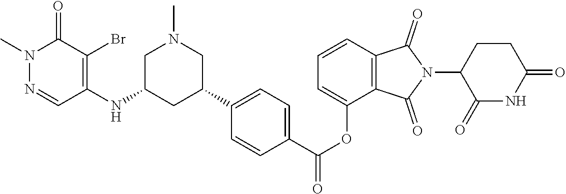 [SMILES: CN1C[C@@H](Nc2cnn(C)c(=O)c2Br)C[C@@H](c2ccc(C(=O)Oc3cccc4c3C(=O)N(C3CCC(=O)NC3=O)C4=O)cc2)C1] |
| | 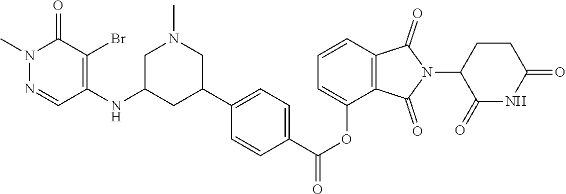 [SMILES: CN1CC(Nc2cnn(C)c(=O)c2Br)CC(c2ccc(C(=O)Oc3cccc4c3C(=O)N(C3CCC(=O)NC3=O)C4=O)cc2)C1] |
| | 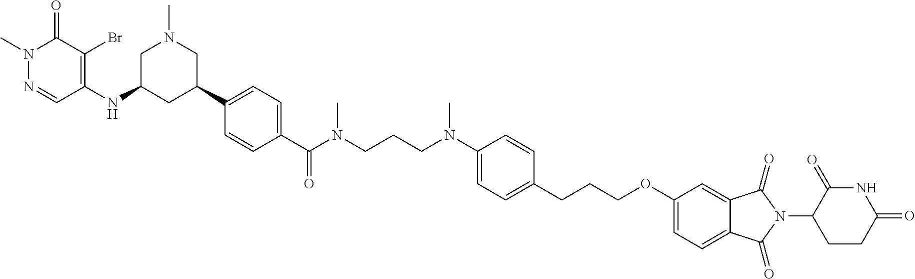 [SMILES: CN1C[C@H](Nc2cnn(C)c(=O)c2Br)C[C@H](c2ccc(C(=O)N(C)CCCN(C)c3ccc(CCCOc4ccc5c(c4)C(=O)N(C4CCC(=O)NC4=O)C5=O)cc3)cc2)C1] |
| | 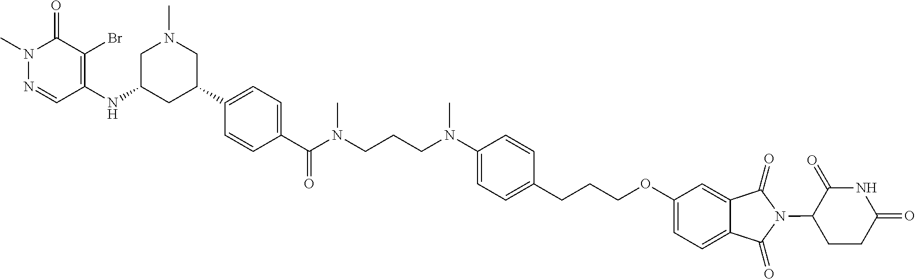 [SMILES: CN1C[C@@H](Nc2cnn(C)c(=O)c2Br)C[C@@H](c2ccc(C(=O)N(C)CCCN(C)c3ccc(CCCOc4ccc5c(c4)C(=O)N(C4CCC(=O)NC4=O)C5=O)cc3)cc2)C1] |
| | 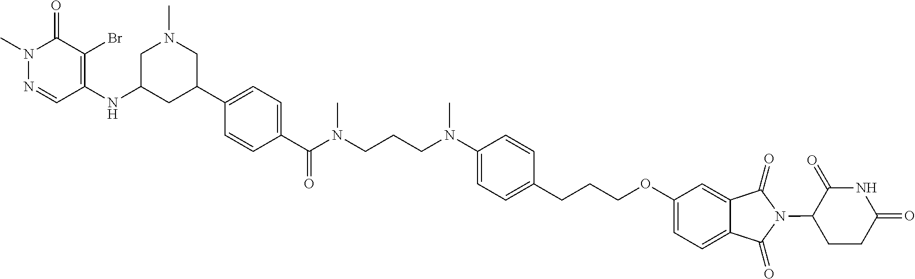 [SMILES: CN1CC(Nc2cnn(C)c(=O)c2Br)CC(c2ccc(C(=O)N(C)CCCN(C)c3ccc(CCCOc4ccc5c(c4)C(=O)N(C4CCC(=O)NC4=O)C5=O)cc3)cc2)C1] |
| | 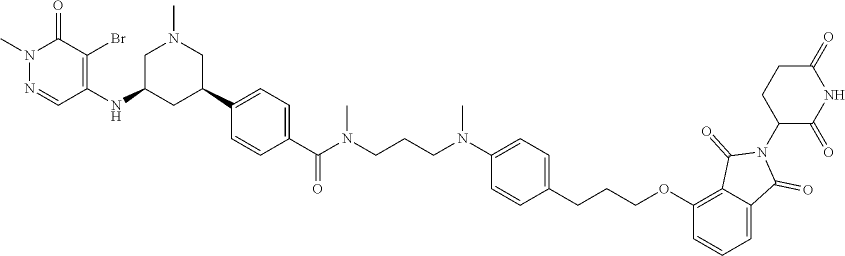 [SMILES: CN1C[C@H](Nc2cnn(C)c(=O)c2Br)C[C@H](c2ccc(C(=O)N(C)CCCN(C)c3ccc(CCCOc4cccc5c4C(=O)N(C4CCC(=O)NC4=O)C5=O)cc3)cc2)C1] |
| | 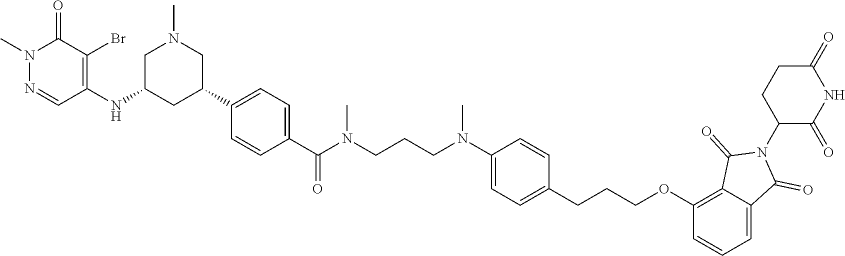 [SMILES: CN1C[C@@H](Nc2cnn(C)c(=O)c2Br)C[C@@H](c2ccc(C(=O)N(C)CCCN(C)c3ccc(CCCOc4cccc5c4C(=O)N(C4CCC(=O)NC4=O)C5=O)cc3)cc2)C1] |
| | 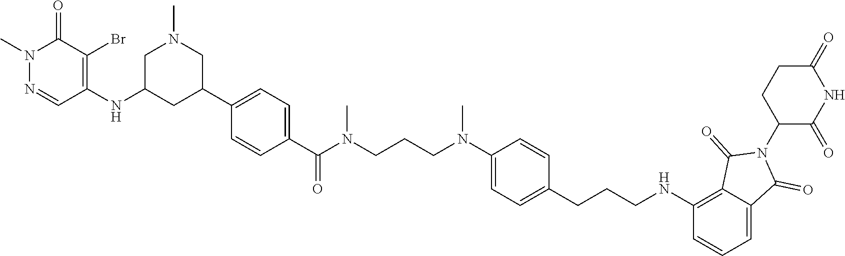 [SMILES: CN1CC(Nc2cnn(C)c(=O)c2Br)CC(c2ccc(C(=O)N(C)CCCN(C)c3ccc(CCCNc4cccc5c4C(=O)N(C4CCC(=O)NC4=O)C5=O)cc3)cc2)C1] |
| | 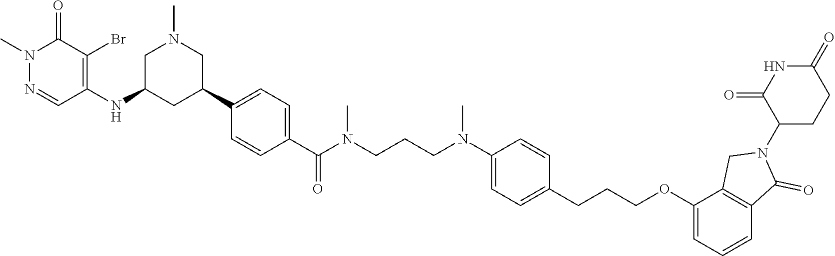 [SMILES: CN1C[C@H](Nc2cnn(C)c(=O)c2Br)C[C@H](c2ccc(C(=O)N(C)CCCN(C)c3ccc(CCCOc4cccc5c4CN(C4CCC(=O)NC4=O)C5=O)cc3)cc2)C1] |
| | 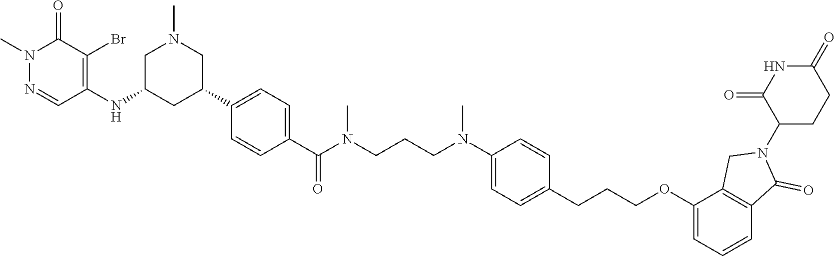 [SMILES: CN1C[C@@H](Nc2cnn(C)c(=O)c2Br)C[C@@H](c2ccc(C(=O)N(C)CCCN(C)c3ccc(CCCOc4cccc5c4CN(C4CCC(=O)NC4=O)C5=O)cc3)cc2)C1] |
| | 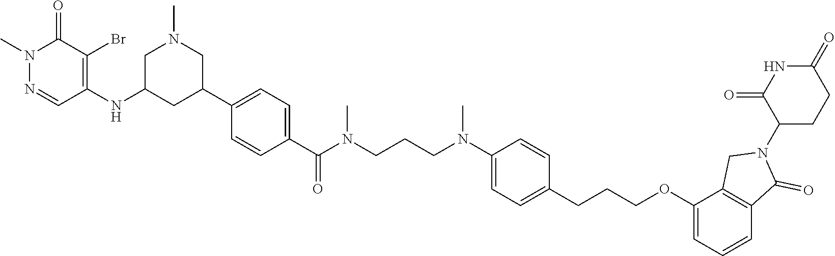 [SMILES: CN1CC(Nc2cnn(C)c(=O)c2Br)CC(c2ccc(C(=O)N(C)CCCN(C)c3ccc(CCCOc4cccc5c4CN(C4CCC(=O)NC4=O)C5=O)cc3)cc2)C1] |
| | 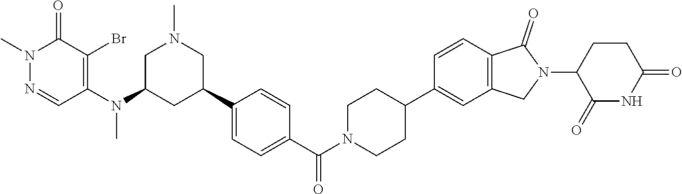 [SMILES: CN1C[C@@H](c2ccc(C(=O)N3CCC(c4ccc5c(c4)CN(C4CCC(=O)NC4=O)C5=O)CC3)cc2)C[C@@H](N(C)c2cnn(C)c(=O)c2Br)C1] |
| | 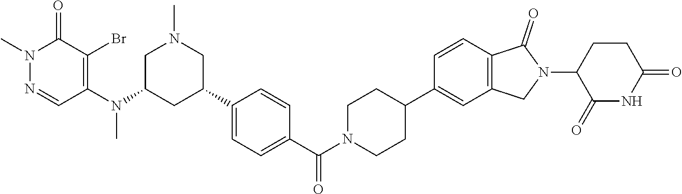 [SMILES: CN1C[C@H](c2ccc(C(=O)N3CCC(c4ccc5c(c4)CN(C4CCC(=O)NC4=O)C5=O)CC3)cc2)C[C@H](N(C)c2cnn(C)c(=O)c2Br)C1] |
| | 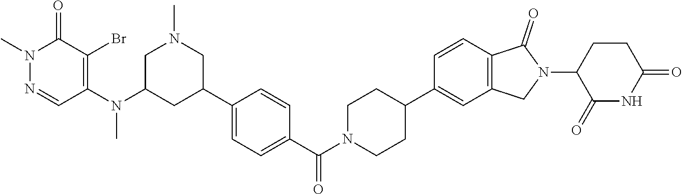 [SMILES: CN1CC(c2ccc(C(=O)N3CCC(c4ccc5c(c4)CN(C4CCC(=O)NC4=O)C5=O)CC3)cc2)CC(N(C)c2cnn(C)c(=O)c2Br)C1] |
| | 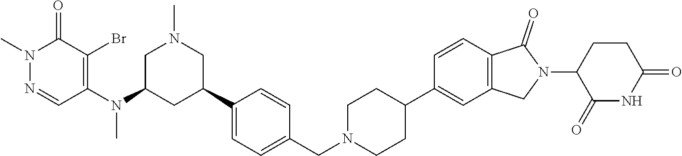 [SMILES: CN1C[C@@H](c2ccc(CN3CCC(c4ccc5c(c4)CN(C4CCC(=O)NC4=O)C5=O)CC3)cc2)C[C@@H](N(C)c2cnn(C)c(=O)c2Br)C1] |
| | 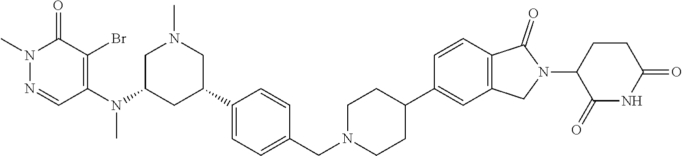 [SMILES: CN1C[C@H](c2ccc(CN3CCC(c4ccc5c(c4)CN(C4CCC(=O)NC4=O)C5=O)CC3)cc2)C[C@H](N(C)c2cnn(C)c(=O)c2Br)C1] |
| | 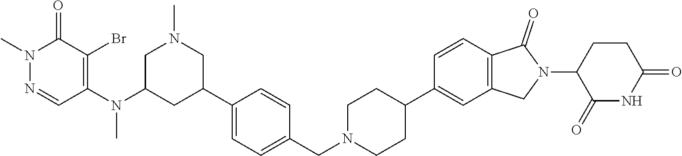 [SMILES: CN1CC(c2ccc(CN3CCC(c4ccc5c(c4)CN(C4CCC(=O)NC4=O)C5=O)CC3)cc2)CC(N(C)c2cnn(C)c(=O)c2Br)C1] |
| | 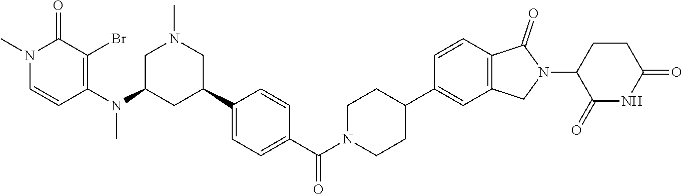 [SMILES: CN1C[C@@H](c2ccc(C(=O)N3CCC(c4ccc5c(c4)CN(C4CCC(=O)NC4=O)C5=O)CC3)cc2)C[C@@H](N(C)c2ccn(C)c(=O)c2Br)C1] |
| | 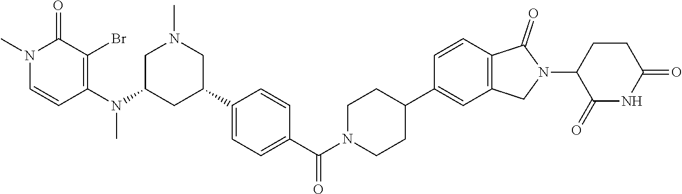 [SMILES: CN1C[C@H](c2ccc(C(=O)N3CCC(c4ccc5c(c4)CN(C4CCC(=O)NC4=O)C5=O)CC3)cc2)C[C@H](N(C)c2ccn(C)c(=O)c2Br)C1] |
| | 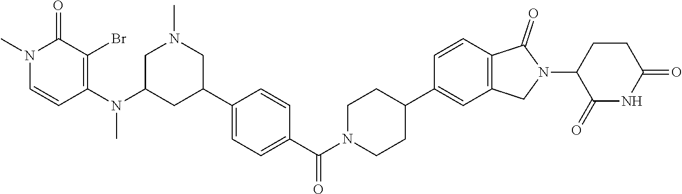 [SMILES: CN1CC(c2ccc(C(=O)N3CCC(c4ccc5c(c4)CN(C4CCC(=O)NC4=O)C5=O)CC3)cc2)CC(N(C)c2ccn(C)c(=O)c2Br)C1] |
| | 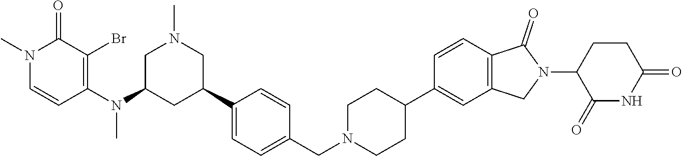 [SMILES: CN1C[C@@H](c2ccc(CN3CCC(c4ccc5c(c4)CN(C4CCC(=O)NC4=O)C5=O)CC3)cc2)C[C@@H](N(C)c2ccn(C)c(=O)c2Br)C1] |
| | 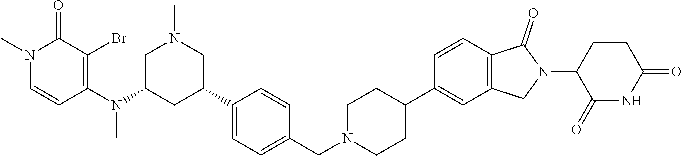 [SMILES: CN1C[C@H](c2ccc(CN3CCC(c4ccc5c(c4)CN(C4CCC(=O)NC4=O)C5=O)CC3)cc2)C[C@H](N(C)c2ccn(C)c(=O)c2Br)C1] |
| | 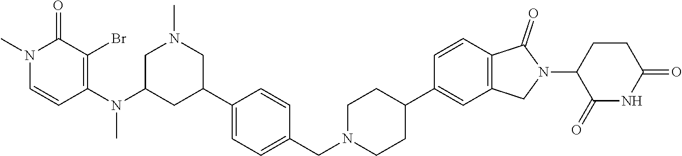 [SMILES: CN1CC(c2ccc(CN3CCC(c4ccc5c(c4)CN(C4CCC(=O)NC4=O)C5=O)CC3)cc2)CC(N(C)c2ccn(C)c(=O)c2Br)C1] |
| | 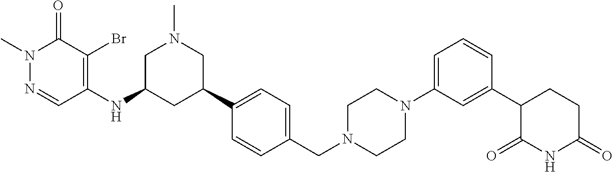 [SMILES: CN1C[C@H](Nc2cnn(C)c(=O)c2Br)C[C@H](c2ccc(CN3CCN(c4cccc(C5CCC(=O)NC5=O)c4)CC3)cc2)C1] |
| | 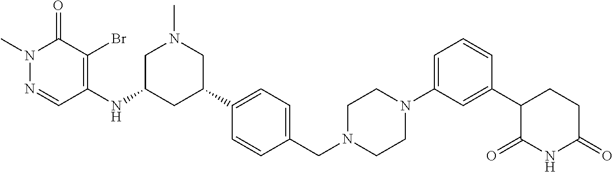 [SMILES: CN1C[C@@H](Nc2cnn(C)c(=O)c2Br)C[C@@H](c2ccc(CN3CCN(c4cccc(C5CCC(=O)NC5=O)c4)CC3)cc2)C1] |
| | 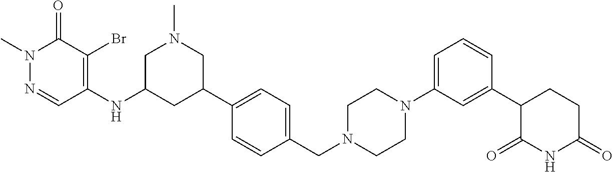 [SMILES: CN1CC(Nc2cnn(C)c(=O)c2Br)CC(c2ccc(CN3CCN(c4cccc(C5CCC(=O)NC5=O)c4)CC3)cc2)C1] |
| | 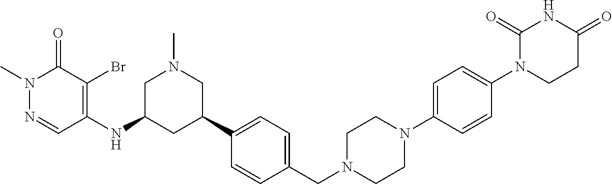 [SMILES: CN1C[C@H](Nc2cnn(C)c(=O)c2Br)C[C@H](c2ccc(CN3CCN(c4ccc(N5CCC(=O)NC5=O)cc4)CC3)cc2)C1] |
| | 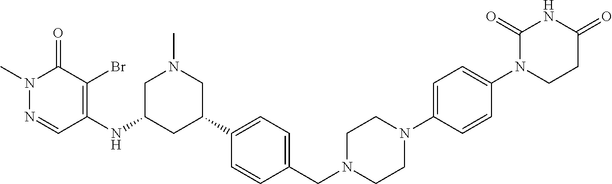 [SMILES: CN1C[C@@H](Nc2cnn(C)c(=O)c2Br)C[C@@H](c2ccc(CN3CCN(c4ccc(N5CCC(=O)NC5=O)cc4)CC3)cc2)C1] |
| | 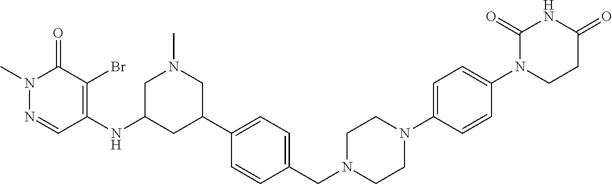 [SMILES: CN1CC(Nc2cnn(C)c(=O)c2Br)CC(c2ccc(CN3CCN(c4ccc(N5CCC(=O)NC5=O)cc4)CC3)cc2)C1] |
| | 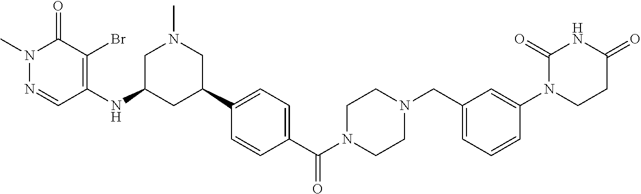 [SMILES: CN1C[C@H](Nc2cnn(C)c(=O)c2Br)C[C@H](c2ccc(C(=O)N3CCN(Cc4cccc(N5CCC(=O)NC5=O)c4)CC3)cc2)C1] |
| | 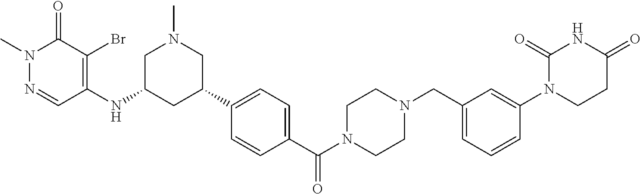 [SMILES: CN1C[C@@H](Nc2cnn(C)c(=O)c2Br)C[C@@H](c2ccc(C(=O)N3CCN(Cc4cccc(N5CCC(=O)NC5=O)c4)CC3)cc2)C1] |
| | 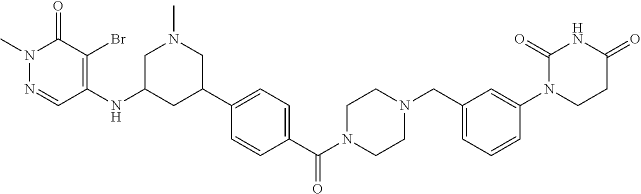 [SMILES: CN1CC(Nc2cnn(C)c(=O)c2Br)CC(c2ccc(C(=O)N3CCN(Cc4cccc(N5CCC(=O)NC5=O)c4)CC3)cc2)C1] |
| | 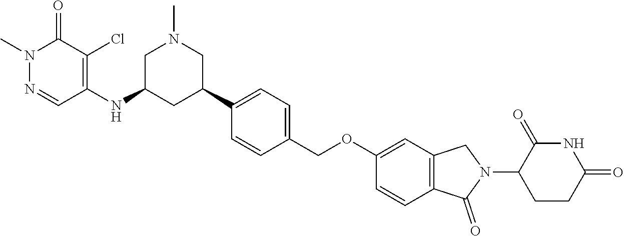 [SMILES: CN1C[C@H](Nc2cnn(C)c(=O)c2Cl)C[C@H](c2ccc(COc3ccc4c(c3)CN(C3CCC(=O)NC3=O)C4=O)cc2)C1] |
| | 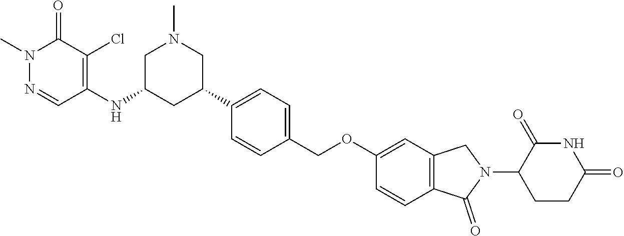 [SMILES: CN1C[C@@H](Nc2cnn(C)c(=O)c2Cl)C[C@@H](c2ccc(COc3ccc4c(c3)CN(C3CCC(=O)NC3=O)C4=O)cc2)C1] |
| | 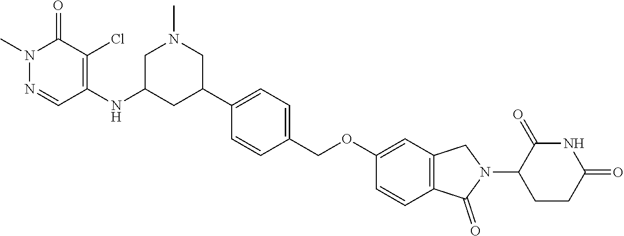 [SMILES: CN1CC(Nc2cnn(C)c(=O)c2Cl)CC(c2ccc(COc3ccc4c(c3)CN(C3CCC(=O)NC3=O)C4=O)cc2)C1] |
| 2-44 | 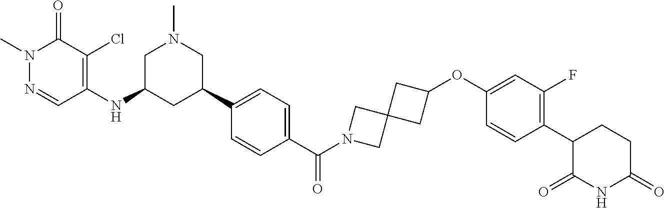 [SMILES: CN1C[C@H](Nc2cnn(C)c(=O)c2Cl)C[C@H](c2ccc(C(=O)N3CC4(CC(Oc5ccc(C6CCC(=O)NC6=O)c(F)c5)C4)C3)cc2)C1] |
| | 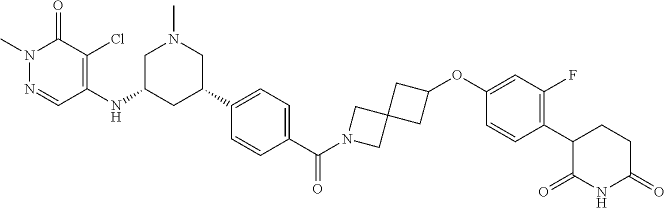 [SMILES: CN1C[C@@H](Nc2cnn(C)c(=O)c2Cl)C[C@@H](c2ccc(C(=O)N3CC4(CC(Oc5ccc(C6CCC(=O)NC6=O)c(F)c5)C4)C3)cc2)C1] |
| | 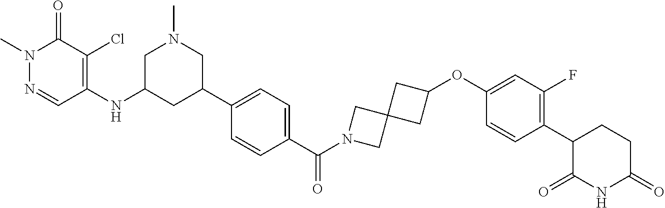 [SMILES: CN1CC(Nc2cnn(C)c(=O)c2Cl)CC(c2ccc(C(=O)N3CC4(CC(Oc5ccc(C6CCC(=O)NC6=O)c(F)c5)C4)C3)cc2)C1] |
| 2-52 | 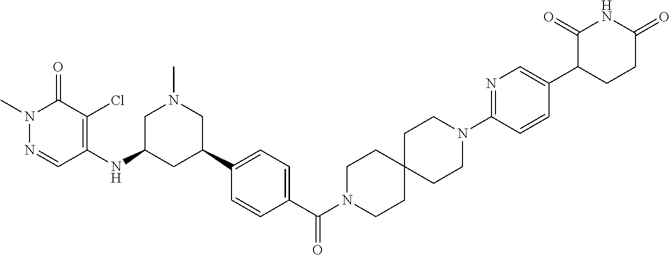 [SMILES: CN1C[C@H](Nc2cnn(C)c(=O)c2Cl)C[C@H](c2ccc(C(=O)N3CCC4(CC3)CCN(c3ccc(C5CCC(=O)NC5=O)cn3)CC4)cc2)C1] |
| | 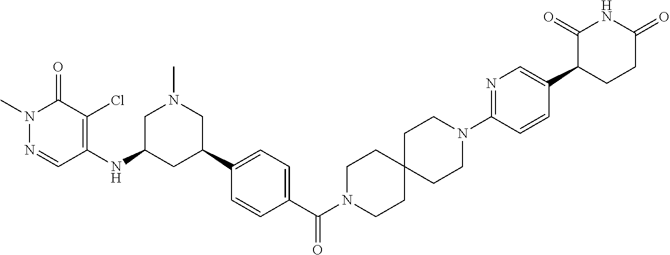 [SMILES: CN1C[C@H](Nc2cnn(C)c(=O)c2Cl)C[C@H](c2ccc(C(=O)N3CCC4(CC3)CCN(c3ccc([C@@H]5CCC(=O)NC5=O)cn3)CC4)cc2)C1] |
| | 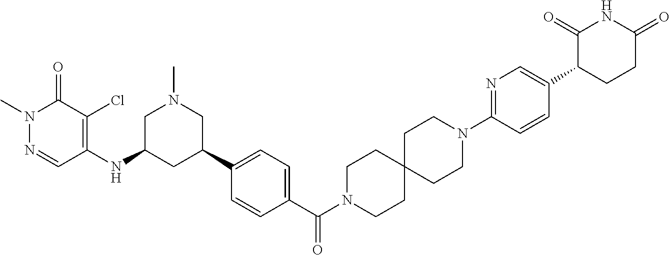 [SMILES: CN1C[C@H](Nc2cnn(C)c(=O)c2Cl)C[C@H](c2ccc(C(=O)N3CCC4(CC3)CCN(c3ccc([C@H]5CCC(=O)NC5=O)cn3)CC4)cc2)C1] |
| | 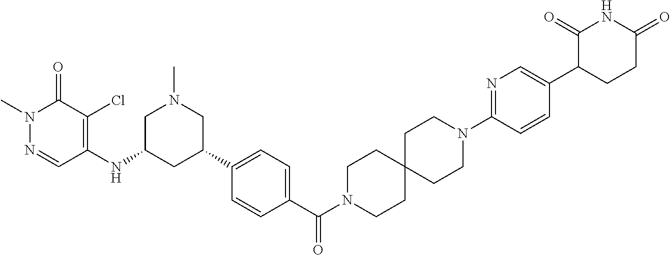 [SMILES: CN1C[C@@H](Nc2cnn(C)c(=O)c2Cl)C[C@@H](c2ccc(C(=O)N3CCC4(CC3)CCN(c3ccc(C5CCC(=O)NC5=O)cn3)CC4)cc2)C1] |
| | 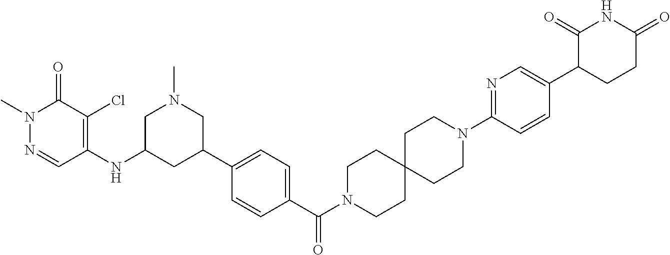 [SMILES: CN1CC(Nc2cnn(C)c(=O)c2Cl)CC(c2ccc(C(=O)N3CCC4(CC3)CCN(c3ccc(C5CCC(=O)NC5=O)cn3)CC4)cc2)C1] |
| 2-69 | 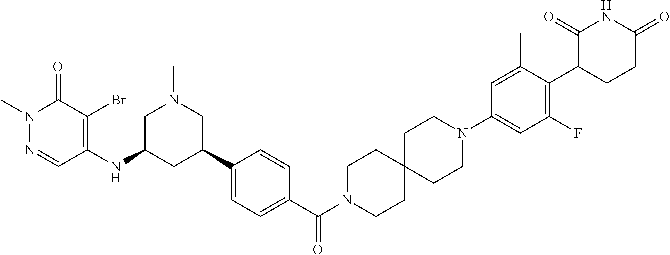 [SMILES: Cc1cc(N2CCC3(CCN(C(=O)c4ccc([C@H]5C[C@@H](Nc6cnn(C)c(=O)c6Br)CN(C)C5)cc4)CC3)CC2)cc(F)c1C1CCC(=O)NC1=O] |
| | 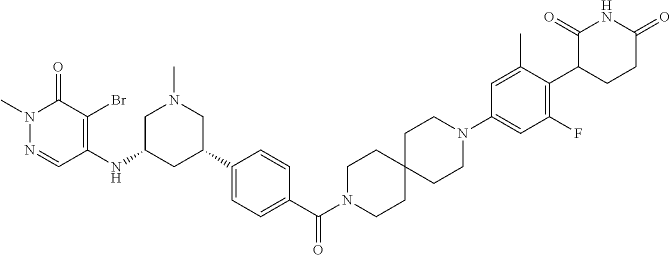 [SMILES: Cc1cc(N2CCC3(CCN(C(=O)c4ccc([C@@H]5C[C@H](Nc6cnn(C)c(=O)c6Br)CN(C)C5)cc4)CC3)CC2)cc(F)c1C1CCC(=O)NC1=O] |
| | 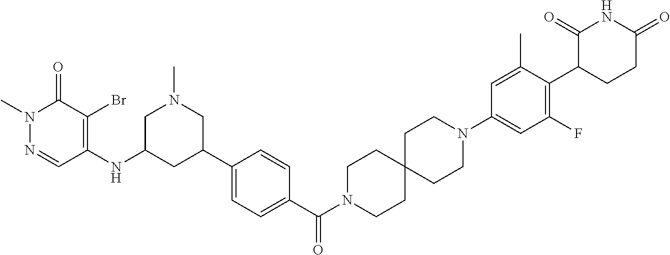 [SMILES: Cc1cc(N2CCC3(CCN(C(=O)c4ccc(C5CC(Nc6cnn(C)c(=O)c6Br)CN(C)C5)cc4)CC3)CC2)cc(F)c1C1CCC(=O)NC1=O] |

In some embodiments, the present disclosure encompasses the recognition that provided compounds display certain desirable characteristics, e.g., as compared to other known compounds (such as GSK983, GSK699, and/or GSK702, described in Bassi, Z. I., et al. ACS Chem. Biol., 2018, 13, 2862-67). For example, in some embodiments, provided compounds are more potent than certain known compounds in one or more assays described herein, e.g., the Western Blot assay of Example B1. In some embodiments, provided compounds are more soluble than certain known compounds, as measured by, e.g., kinetic and/or thermodynamic solubility assays. In some embodiments, provided compounds have improved metabolic stability than certain known compounds, as measured by, e.g., intrinsic clearance using an in vitro liver microsomal stability assay and/or an in vivo pharmacokinetic analysis. In some embodiments, provided compounds have improved permeability, as measured by, e.g., a MDCK and/or Caco2 permeability assays. Without wishing to be bound by theory, it will be appreciated that provided compounds that display a suitable balance of any two or more of these properties may be particularly suitable for development as a drug, as evidenced, in some embodiments, by pharmacokinetic properties, such as AUC, T_{1/2}, MRT, or CL.

In some embodiments, the present disclosure encompasses the recognition that provided compounds are capable of degrading KAT2A, KAT2B, or both KAT2A and KAT2B. In some embodiments, it will be appreciated that compounds capable of degrading both KAT2A and KAT2B provide benefits over compounds that only degrade one of KAT2A or KAT2B. In other embodiments, compounds that selectively degrade KAT2A over KAT2B may be desirable. In other embodiments, compounds that selectively degrade KAT2B over KAT2A may be desirable.

In some embodiments, provided compounds are provided and/or utilized in a salt form (e.g., a pharmaceutically acceptable salt form). Reference to a compound provided herein is understood to include reference to salts thereof, unless otherwise indicated. Pharmaceutically acceptable salt forms are known in the art. For example, S. M. Berge, et al. describes pharmaceutically acceptable salts in detail in J. Pharmaceutical Sciences, 66:1-19(1977).

It will be appreciated that throughout the present disclosure, unless otherwise indicated, reference to a compound of Formula I is intended to also include Formulae II, IIA, IIA-1, IIA-2, IIA-3, IIA-4, IIA-5, IIA-6, III, IIIA, IIIB, IIIB-1, IIIB-2, IIIC, IIID, IV, IVA, IVA-1, V, VA, VA-1, VB, VB-1, VI, VIA, VIA-1, VII, VIIA, VIII, VIIIA, IX, IXA, IXB, IXC, IXD, X, XA, XA-1, XA-2, XB, XI, XIA, XII, XIIA, XIIB, XIIB-1, XIII, XIIIA, XIV, and XV, and compound species of such formulas disclosed herein.

### Manufacturing

Provided compounds may generally be made by the processes described in the ensuing schemes and examples. In some embodiments, provided compounds (e.g., compounds with a linker comprising an amide) are prepared according to the following Scheme:

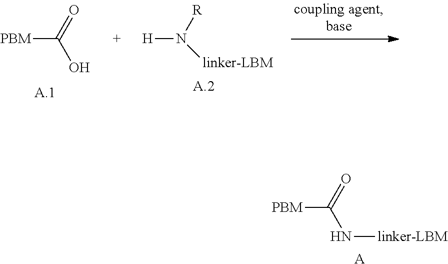

wherein PBM, linker, LBM, and R are as defined in Formulae herein. Accordingly, in some embodiments, compound A is prepared by a process comprising contacting intermediate A.1 with intermediate A.2 in the presence of a suitable coupling agent (e.g., N,N,N′,N′-tetramethylchloroformamidinium hexafluorophosphate), optionally in the presence of a suitable base (e.g., N-methylimidazole). In some embodiments, intermediate A.1 has the following structure:

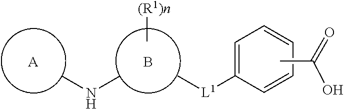

In some embodiments, intermediate A.1 has the following structure:

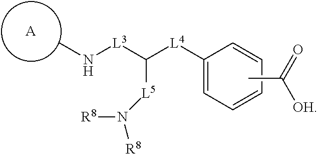

In some embodiments, provided compounds (e.g., compounds with a linker comprising an amide) are prepared according to the following Scheme:

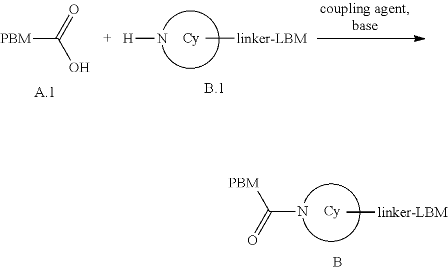

wherein PBM, linker, LBM, and Cy are as defined in Formulae herein. Accordingly, in some embodiments, compound B is prepared by a process comprising contacting intermediate A.1 with intermediate B.1 in the presence of a suitable coupling agent (e.g., N,N,N′,N′-tetramethylchloroformamidinium hexafluorophosphate), optionally in the presence of a suitable base (e.g., N-methylimidazole). In some embodiments, intermediate A.1 has the following structure:

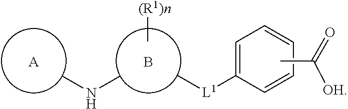

In some embodiments, intermediate A.1 has the following structure:

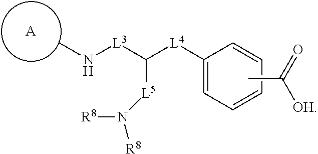

In some embodiments, provided compounds (e.g., compounds with a linker comprising an ester) are prepared according to the following Scheme:

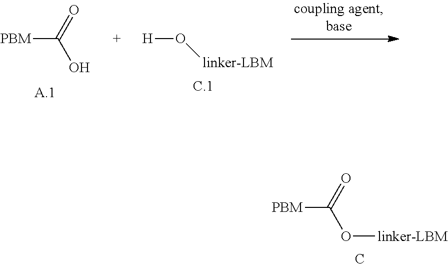

wherein PBM, linker, and LBM are as defined in Formulae herein. Accordingly, in some embodiments, compound C is prepared by a process comprising contacting intermediate A.1 with intermediate C.1 in the presence of a suitable coupling agent (e.g., N,N′-diisopropylcarbodiimide), optionally in the presence of a suitable base (e.g., 4-dimethylaminopyridine). In some embodiments, intermediate A.1 has the following structure:

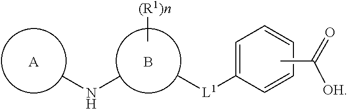

In some embodiments, intermediate A.1 has the following structure:

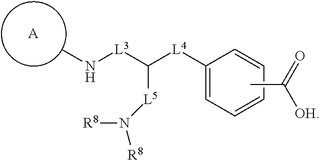

In some embodiments, provided compounds are prepared according to the following Scheme:

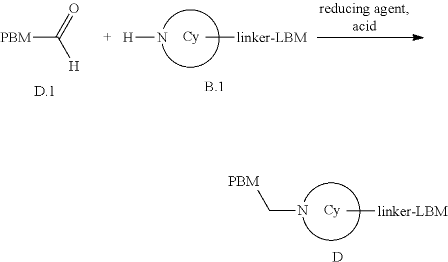

wherein PBM, linker, LBM, and Cy are as defined in Formulae herein. Accordingly, in some embodiments, compound D is prepared by a process comprising contacting intermediate D.1 with intermediate B.1 in the presence of a suitable reducing agent (e.g., NaCNBH₃), optionally in the presence of a suitable acid (e.g., AcOH). In some embodiments, intermediate D.1 has the following structure:

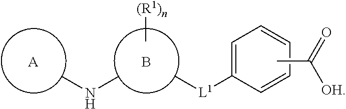

In some embodiments, intermediate D.1 has the following structure:

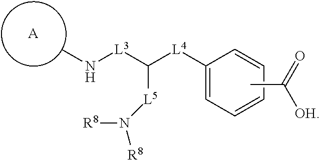

In some embodiments, provided compounds are prepared according to one of the following Schemes:

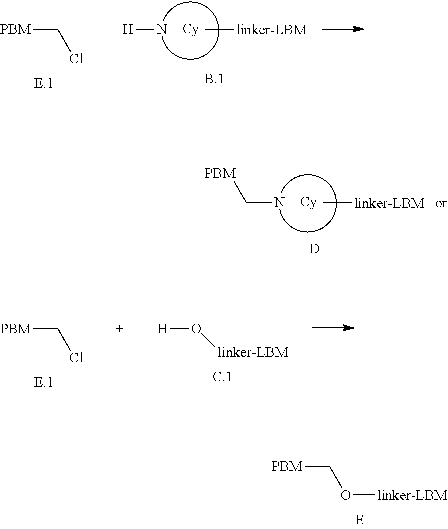

wherein PBM, linker, LBM, and Cy are as defined in Formulae herein. Accordingly, in some embodiments, compound D is prepared by a process comprising contacting intermediate E.1 with intermediate B.1, optionally in the presence of a suitable base (e.g., Cs₂CO₃). In some embodiments, compound E is prepared by a process comprising contacting intermediate E.1 with intermediate C.1, optionally in the presence of a suitable base (e.g., Cs₂CO₃). In some embodiments, intermediate E.1 has the following structure:

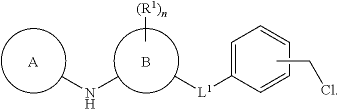

In some embodiments, intermediate E.1 has the following structure:

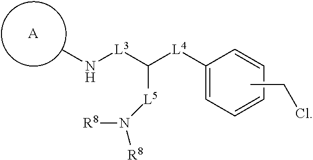

In some embodiments, provided compounds (e.g., compounds with a linker comprising a triazole ring) are prepared according to the following Scheme:

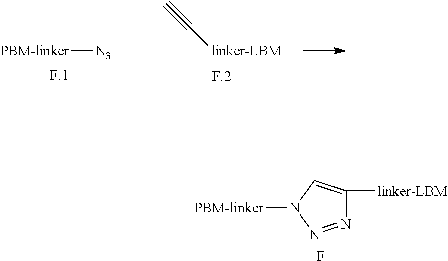

wherein PBM, linker, and LBM are as defined in Formulae herein. Accordingly, in some embodiments, compound F is prepared by a process comprising contacting intermediate F.1 with intermediate F.2, optionally in the presence of a suitable catalyst (e.g., a copper catalyst). In some embodiments, intermediate F.1 has the following structure:

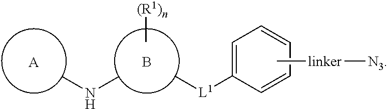

In some embodiments, intermediate F.1 has the following structure:

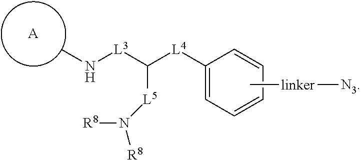

In some embodiments, the present disclosure provides compounds of high purity. In some embodiments, purity of a compound is or greater than about 85%, 90%, 91%, 92%, 93%, 94%, or 95%. In some embodiments, purity of a compound is or greater than about 80%. In some embodiments, purity of a compound is or greater than about 85%. In some embodiments, purity of a compound is or greater than about 90%. In some embodiments, purity of a compound is or greater than about 95%. In some embodiments, a percentage is wt %. In some embodiments, a percentage is peak area %, e.g., from a separation technology (e.g., HPLC, UPLC, or GC) which can separate a compound from impurities using a suitable detection technology (e.g., UV at one or more lengths (e.g., 220 nm or 254 nm), MS, or MS″). In some embodiments, purity is assessed using quantitative NMR (qNMR). In some embodiments, a qNMR technology utilizes an internal standard. In some embodiments, an internal standard is of suitable purity, e.g., at least about 99%, 99.5%, 99.6%, 99.7%, 99.8%, 99.9%, 99.99%, etc. (wt % and/or mol %). In some embodiments, a composition of a compound (e.g., a drug substance preparation of a compound, or a pharmaceutical composition comprising a compound) when assessed using a LC technology has a peak area purity of about or at least about 95%, e.g., when using UV detection at a suitable wavelength (e.g., 220 nm or 254 nm). Alternatively or additionally, in some embodiments, a composition of a compound (e.g., a drug substance preparation of a compound, or a pharmaceutical composition comprising a compound) when assessed using a qNMR technology has a purity (e.g., wt %) of about or at least about 95%, and/or about or no more than about 105%.

In some embodiments, the present disclosure provides compounds of high stereochemical purity. In some embodiments, stereochemical purity of a compound is or greater than about 85%, 90%, 91%, 92%, 93%, 94%, or 95%. In some embodiments, stereochemical purity of a compound is or greater than about 80%. In some embodiments, stereochemical purity of a compound is or greater than about 85%. In some embodiments, stereochemical purity of a compound is or greater than about 90%. In some embodiments, stereochemical purity of a compound is or greater than about 95%. In some embodiments, stereochemical purity is the percentage of the peak area of one stereoisomer over the total peak areas of all stereoisomers, e.g., when detected using UV (e.g., at certain wavelength such as 220 nm or 254 nm). In some embodiments, stereochemical purity of a stereoisomer is assessed by NMR. In some embodiments, stereochemical purity is performed on a sample having high purity (e.g., as assessed using technologies described herein (e.g., in some embodiments, LC-UV and/or qNMR).

In some embodiments, the present disclosure provides compounds of high enantiomeric purity. In some embodiments, enantiomeric purity of a compound is or greater than about 85%, 90%, 91%, 92%, 93%, 94%, or 95%. In some embodiments, enantiomeric purity of a compound is or greater than about 80%. In some embodiments, enantiomeric purity of a compound is or greater than about 85%. In some embodiments, enantiomeric purity of a compound is or greater than about 90%. In some embodiments, enantiomeric purity of a compound is or greater than about 95%.

In some embodiments, the present disclosure provides compounds of high diastereomeric purity. In some embodiments, diastereomeric purity of a compound is or greater than about 85%, 90%, 91%, 92%, 93%, 94%, or 95%. In some embodiments, diastereomeric purity of a compound is or greater than about 80%. In some embodiments, diastereomeric purity of a compound is or greater than about 85%. In some embodiments, diastereomeric purity of a compound is or greater than about 90%. In some embodiments, diastereomeric purity of a compound is or greater than about 95%.

Stereochemically pure, e.g., enantiomerically pure or diastereomerically pure, compounds and compositions can be prepared utilizing various technologies in accordance with the present disclosure. For example, in some embodiments, they can be prepared through separation including chiral separation; in some embodiments, they can be prepared through stereoselective synthesis. In some embodiments, enantiomeric purity is the percentage of the peak area of one enantiomer over the total peak areas of both enantiomers, e.g., when detected using UV (e.g., at certain wavelength such as 220 nm, 254 nm, etc.). In some embodiments, enantiomeric purity of an enantiomer is assessed by NMR (e.g., in a chiral solvent, in the present of a chiral agent that interact with an enantiomer). In some embodiments, enantiomeric purity is performed on a sample having high purity (e.g., as assessed using technologies described herein (e.g., in some embodiments, LC-UV and/or qNMR). In some embodiments, diastereomeric purity is the percentage of the peak area of one diastereomer over the total peak areas of all diastereomers, e.g., when detected using UV (e.g., at certain wavelength such as 220 nm or 254 nm). In some embodiments, diastereomeric purity of a diastereomer is assessed by NMR (e.g., in a chiral solvent, in the present of a chiral agent that interact with an diastereomer). In some embodiments, diastereomeric purity is performed on a sample having high purity (e.g., as assessed using technologies described herein (e.g., in some embodiments, LC-UV and/or qNMR).

### Compositions

The present disclosure also provides compositions that comprise or deliver a compound as provided herein. In some embodiments, the present disclosure provides compositions comprising a compound provided herein with one or more other components. As used herein, the term “pharmaceutical composition” refers to a composition in which an active agent is formulated together with one or more pharmaceutically acceptable carriers. In some embodiments, the active agent is present in unit dose amount appropriate for administration in a therapeutic regimen that shows a statistically significant probability of achieving a predetermined therapeutic effect when administered to a relevant population. In some embodiments, a pharmaceutical composition may be specially formulated for administration in a particular form (e.g., in a solid form or a liquid form), and/or may be specifically adapted for, for example: oral administration (for example, as a drenche [aqueous or non-aqueous solutions or suspensions], tablet, capsule, bolus, powder, granule, paste, etc., which may be formulated specifically for example for buccal, sublingual, or systemic absorption); parenteral administration (for example, by subcutaneous, intramuscular, intravenous or epidural injection as, for example, a sterile solution or suspension, or sustained-release formulation, etc.); topical application (for example, as a cream, ointment, patch or spray applied for example to skin, lungs, or oral cavity); intravaginal or intrarectal administration (for example, as a pessary, suppository, cream, or foam): ocular administration; nasal or pulmonary administration, etc.

In some embodiments, the present disclosure provides compositions comprising a compound provided herein and a pharmaceutically acceptable carrier. In some embodiments, a compound is in a pharmaceutically acceptable salt form. The term “pharmaceutically acceptable salt”, as used herein, refers to salts of such compounds that are appropriate for use in pharmaceutical contexts, i.e., salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66:1-19 (1977). In some embodiments, pharmaceutically acceptable salt include nontoxic acid addition salts, which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. In some embodiments, pharmaceutically acceptable salts include adipate, alginate, ascorbate, aspartate, benzenesulfonate, benzoate, bisulfate, borate, butyrate, camphorate, camphorsulfonate, citrate, cyclopentanopropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In some embodiments, a salt is citrate salt. In some embodiments, a provided compound comprises one or more acidic groups, e.g., an oligonucleotide, and a pharmaceutically acceptable salt is an alkali, alkaline earth metal, or ammonium (e.g., an ammonium salt of N(R)₃, wherein each R is independently defined and described in the present disclosure) salt. Representative alkali or alkaline earth metal salts include sodium, lithium, potassium, calcium, magnesium, and the like. In some embodiments, a pharmaceutically acceptable salt is a sodium salt. In some embodiments, a pharmaceutically acceptable salt is a potassium salt. In some embodiments, a pharmaceutically acceptable salt is a calcium salt. In some embodiments, pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate. In some embodiments, a provided compound comprises more than one acid groups, for example, an oligonucleotide may comprise two or more acidic groups (e.g., in natural phosphate linkages and/or modified internucleotidic linkages). In some embodiments, a pharmaceutically acceptable salt, or generally a salt, of such a compound comprises two or more cations, which can be the same or different.

As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations. In some embodiments, a pharmaceutically acceptable carrier is a buffer. In some embodiments, a pharmaceutical composition comprises a buffer. In some embodiments, a pharmaceutical composition comprises the solid components a buffer. In some embodiments, a pharmaceutical composition is re-constituted by addition of solvent, e.g., water. In some embodiments, a pharmaceutically acceptable carrier is an acidic buffer. In some embodiments, its pH is about 2-6.5, 2-6, 3-6, 2, 2.5, 3, 3.5, 4, 4.5, 5, 5.5, 6 or 6.5. In some embodiments, a pharmaceutically acceptable carrier is a citrate buffer, e.g., pH about 3.5. In some embodiments, a provided compound can be formulated without using compounds such as cyclodextrins (e.g., HPBCD) to improve apparent solubility and/or dissolution rate.

In some embodiments, provided compositions comprise and/or deliver a compound described herein (e.g., compounds of Formulae I, II, IIA, IIA-1, IIA-2, IIA-3, IIA-4, IIA-5, IIA-6, II-1, III, IIIA, IIIB, IIIB-1, IIIB-2, IIIC, IIID, III-1, IIIE, IIIF, IIIG, IIIH, IIIJ, IIIK, IIIL, IIIM, IIIM, IV, IVA, IVA-1, IV-1, IVD, V, VA, VA-1, VB, VB-1, V-1, VC, VD, VE, VI, VIA, VIA-1, VI-1, VIB, VII, VIIA, VII-1, VIIB, VIII, VIIIA, VIII-1, VIIIB, IX, IX-1, IXA, IXB, IXC, IXD, X, XA, XA-1, XA-2, XB, X-1, X-2, X-3, XA′, XA-1′, XA-2′, XI, XIA, CI-1, XIB, XII, XIIA, XIIB, XIIB-1, XII-1, XIIA′, XIIB′, XIIV-1′, XIII, XIIIA, XIII-1, XIIIB, XIV, XIV-1, XV, and XV-1).

In some embodiments, a provided composition is a pharmaceutical composition that comprises and/or delivers a compound provided herein (e.g., compounds of Formulae I, II, IIA, IIA-1, IIA-2, HA-3, DIA-4, IIA-5, IIA-6, II-1, III, IIIA, IIIB, IIIB-1, IIIB-2, IIIC, IIID, III-1, IIIE, IIIF, IIIG, IIIH, IIIJ, IIIK, IIIL, IIIM, IIIM, IV, IVA, IVA-1, IV-1, IVD, V, VA, VA-1, VB, VB-1, V-1, VC, VD, VE, VI, VIA, VIA-1, VI-1, VIB. VII, VIIA, VII-1, VIIB, VIII, VIIIA, VIII-1, VIIIB, IX, IX-1, IXA, IXB, IXC, IXD, X, XA, XA-1, XA-2, XB, X-1, X-2, X-3, XA′, XA-1′, XA-2′, XI, XIA, CI-1, XIB, XII, XIIA, XIIB, XIIB-1, XII-1, XIIA′, XIIB′, XIIV-1′, XIII, XIIIA, XIII-1, XIIIB, XIV, XIV-1, XV, and XV-1) and further comprises a pharmaceutically acceptable carrier.

In some embodiments, a composition, e.g., a pharmaceutical composition, comprises a compound and one or more of its stereoisomers. In some embodiments, stereochemical purity of the compound (% among the compound and all its stereoisomers) is about or at least about 10%, 20%, 30%, 40%, 50%, 60%, 70%, 80%, 90%, or 95%. In some embodiments, it is about 10%, 20%, 30%, 40%, 50%, 60%, 70%, 80%, 90%, or 95% with respect to a specific chiral center (e.g., a Ring E chiral carbon atom at which Ring E is bonded to Ring C). In some embodiments, it is about 10%, 20%, 30%, 40%, 50%, 60%, 70%, 80%, 90%, or 95% with respect to one specific chiral center and is independently about or at least about 90% or 95% with respect to each of the other chiral centers. In some embodiments, stereochemical purity is about 10%-80%, 20%-80%, 30%-70%, 40%-70%, 40%-60%, 40%, 45%, 50%, 55% or 60%. In some embodiments, it is about or at least about 85%. In some embodiments, it is about or at least about 90%. In some embodiments, it is about or at least about 95%.

Pharmaceutical compositions typically contain an active agent (e.g., a compound described herein) in an amount effective to achieve a desired therapeutic effect while avoiding or minimizing adverse side effects. In some embodiments, provided pharmaceutical compositions comprise a compound described herein and one or more fillers, disintegrants, lubricants, glidants, anti-adherents, and/or anti-statics, etc. Provided pharmaceutical compositions can be in a variety of forms including oral dosage forms, topical creams, topical patches, iontophoresis forms, suppository, nasal spray and/or inhaler, eye drops, intraocular injection forms, depot forms, as well as injectable and infusible solutions.

Provided pharmaceutical compositions can be prepared with any appropriate available technologies.

In some embodiments, provided compounds are formulated in a unit dosage form for ease of administration and uniformity of dosage. The expression “unit dosage form” as used herein refers to a physically discrete unit of an active agent (e.g., a compound described herein) for administration to a subject. Typically, each such unit contains a predetermined quantity of active agent. In some embodiments, a unit dosage form contains an entire single dose of the agent. In some embodiments, more than one unit dosage form is administered to achieve a total single dose. In some embodiments, administration of multiple unit dosage forms is required, or expected to be required, in order to achieve an intended effect. A unit dosage form may be, for example, a liquid pharmaceutical composition containing a predetermined quantity of one or more active agents, a solid pharmaceutical composition (e.g., a tablet, a capsule, or the like) containing a predetermined amount of one or more active agents, a sustained release formulation containing a predetermined quantity of one or more active agents, or a drug delivery device containing a predetermined amount of one or more active agents, etc.

Provided compositions may be administered in accordance with a dosing regimen (i.e., that includes a single dose or multiple doses separated from one another in time, administered via a particular route of administration) that is (e.g., has been demonstrated to be) effective for treating (e.g., delaying onset of and/or decreasing incidence and/or intensity of) a disease or disorder, for example as described herein.

The present disclosure also provides methods of preparing pharmaceutical compositions provided herein. In some embodiments, provided methods comprise (i) providing a provided compound or a pharmaceutically acceptable salt thereof; and (ii) formulating the compound with suitable excipients to give a pharmaceutical composition.

### Uses

In some embodiments, present disclosure provides uses for compounds and compositions described herein. In some embodiments, provided compounds and compositions are useful in medicine (e.g., as therapy). In some embodiments, provided compounds and compositions are useful in research as, for example, analytical tools and/or control compounds in biological assays. In some embodiments, a provided compound or a salt thereof is formulated, utilized, provided or administered in a pharmaceutical composition as described herein.

Among other things, the present disclosure encompasses the recognition and KAT2 polypeptides, e.g., KAT2A are associated with various dysregulated cell states of various diseases, disorders, or conditions including cancer. In some embodiments, the present disclosure provides technologies targeting such cell states and associated diseases, disorders, or conditions.

In some embodiments, provided compounds are useful as KAT2 degraders and/or inhibitors. In some embodiments, provided compounds promote degradation of KAT2A. In some embodiments, provided compounds promote degradation of KAT2B.

In some embodiments, a compound binds to KAT2A and an E3 ubiquitin ligase. In some embodiments, a compound binds to KAT2B and an E3 ubiquitin ligase. In some embodiments, a compound binds to KAT2A. KAT2B and an E3 ubiquitin ligase. Various technologies can be utilized for assessing binding. In some embodiments, K_{D} of a compound binding to a target is about or less than about 1 μM in an assay such as surface plasmon resonance (SPR) or isothermal titration calorimetry (ITC).

In some embodiments, the present disclosure provides methods of degrading and/or inhibiting KAT2 (e.g., KAT2A and/or KAT2B), comprising contacting a provided compound with KAT2. In some embodiments, contacting occurs in a cell. In some embodiments, contacting occurs in a subject (e.g., a human subject).

In some embodiments, the present disclosure provides methods for reducing level of KAT2A in a system, comprising administering or delivering to the system an effective amount of a provided compound. In some embodiments, the present disclosure provides methods for reducing level of KAT2B in a system, comprising administering or delivering to the system an effective amount of a provided compound. In some embodiments, the present disclosure provides methods for reducing level of KAT2A and KAT2B in a system, comprising administering or delivering to the system an effective amount of a provided compound. In some embodiments, the present disclosure provides methods for modulating epigenetic state of a system, comprising administering or delivering to the system an effective amount of a provided compound. In some embodiments, the present disclosure provides methods for reducing histone acetylation in a system, comprising administering or delivering to the system an effective amount of a provided compound. In some embodiments, H3K9 acetylation is reduced. In some embodiments, the present disclosure provides methods for inducing or promoting cell differentiation in a system, comprising administering or delivering to the system an effective amount of a provided compound. In some embodiments, the present disclosure provides methods for reducing or blocking de-differentiation in a system, comprising administering or delivering to the system an effective amount of a provided compound. In some embodiments, the present disclosure provides methods for reducing or blocking trans-differentiation in a system, comprising administering or delivering to the system an effective amount of a provided compound. In some embodiments, a system comprises or expresses KAT2A. In some embodiments, a system comprises or expresses KAT2B. In some embodiments, a system comprises or expresses KAT2A and KAT2B. In some embodiments, a system is or comprises a cell. In some embodiments, a system is or comprises a cell expressing KAT2A and/or KAT2B. In some embodiments, a cell is a cell of a disease, disorder, or condition. In some embodiments, a cell is a cancer cell. In some embodiments, a cell is an acute myeloid leukemia cell. In some embodiments, a cell is a small cell lung cancer cell. In some embodiments, a cell is a neuroendocrine prostate cancer cell. In some embodiments, a system is or comprises a tissue. In some embodiments, a system is or comprises an organ. In some embodiments, a system is or comprises a sample. In some embodiments, a system is or comprises an organism. In some embodiments, a system is or comprises a subject. In some embodiments, a system is or comprises an animal. In some embodiments, a system is or comprises a human.

In some embodiments, the present disclosure provides a method for modulating a dysregulated cell state associated with KAT2A and/or KAT2B, comprising administering or delivering to the cell an effective amount of a provided compound. In some embodiments, a dysregulated cell state is associated with a disease, disorder, or condition, e.g., cancer. In some embodiments, after modulation a cell state is not or less associated with a disease, disorder, or condition. For example, in some embodiments, a dysregulated cell state is associated with or of a cancer as described herein. In some embodiments, a dysregulated cell state is associated with or of acute myeloid leukemia. In some embodiments, a dysregulated cell state is associated with or of small cell lung cancer. In some embodiments, a dysregulated cell state is associated with or of neuroendocrine prostate cancer. In some embodiments, a dysregulated cell state is or comprises a dedifferentiated neuroendocrine cell state. In some embodiments, a dysregulated cell state is or comprises blocked or reduced differentiation. In some embodiments, dysregulated cell state is or comprises de-differentiation. In some embodiments, dysregulated cell state is or comprises trans-differentiation. In some embodiments, a dysregulated cell state is or comprises a dedifferentiated neuroendocrine cell state. In some embodiments, a provided technology induces or promotes conversion of a dysregulated cell state into another cell state. In some embodiments, a dysregulated cell state is more associated with a condition, disease or disorder, e.g., a cancer as described herein, than another cell state. In some embodiments, a dysregulated cell state is more proliferative than another cell state. In some embodiments, a dysregulated cell state is less differentiated than another cell state. In some embodiments, another cell state is or comprises a more differentiated epithelial cell state. In some embodiments, another cell state comprises more differentiation. In some embodiments, another cell state comprises more terminal differentiation. In some embodiments, another cell state comprises more myeloid differentiation. In some embodiments, another cell state comprises more monocytic differentiation. In some embodiments, another cell state comprises increased level of a marker associated with a cell state. In some embodiments, another cell state comprises increased level of a marker associated with differentiation. In some embodiments, another cell state comprises increased level of CD11b expression. In some embodiments, another cell state comprises increased levels of CD14 expression. In some embodiments, another cell state comprises increased levels of CD15 expression. In some embodiments, another cell state comprises increased levels of CD86 expression. In some embodiments, another cell state comprises decreased stemness, translation, and/or replication. In some embodiments, another cell state comprises decreased stemness. In some embodiments, another cell state comprises decreased translation. In some embodiments, another cell state comprises decreased replication. In some embodiments, another cell state comprises decreased stemness, translation, and/or replication GESA signatures. In some embodiments, another cell state comprises decreased stem GSEA signatures. In some embodiments, another cell state comprises decreased translation GSEA signatures. In some embodiments, another cell state comprises decreased replication GSEA signatures. In some embodiments, another cell state comprises increased myeloid differentiation. In some embodiments, another cell state comprises increased myeloid differentiation GSEA signatures. In some embodiments, another cell state comprises increased monocytic differentiation. In some embodiments, another cell state comprises increased monocytic differentiation GSEA signatures. In some embodiments, another cell state comprises increased epithelial differentiation. In some embodiments, another cell state comprises increased epithelial differentiation GSEA signatures. In some embodiments, another cell state comprises epithelial cell state. In some embodiments, another cell state comprises increased neural differentiation. In some embodiments, another cell state comprises neural cell state. In some embodiments, another cell state comprises increased epithelial neural GSEA signatures. In some embodiments, a provided method increases myeloid differentiation. In some embodiments, a provided method decreases stemness. In some embodiments, a provided method decreases translation. In some embodiments, a provided method decreases replication. In some embodiments, a provided method decreases stem, translation and/or replication GSEA signatures. In some embodiments, a method increases epithelial differentiation. In some embodiments, a method increases epithelial GESA signatures. In some embodiments, a method increases neural differentiation. In some embodiments, a method increases neural GESA signatures. In some embodiments, a provided method increases myeloid differentiation. In some embodiments, a provided method increases myeloid GSEA signatures. In some embodiments, a provided method increases monocytic differentiation. In some embodiments, a provided method increases monocytic GSEA signatures. In some embodiments, a method increases level of CD11b⁺ cells. In some embodiments, a method increases level of CD14⁺ cells. In some embodiments, a method increases level of CD15⁺ cells. In some embodiments, a method increases level of CD86⁺ cells.

In some embodiments, a compound promote degradation of one target more than the other. In some embodiments, certain compounds have selectivity for KAT2A over KAT2B. In some embodiments, certain compounds have selectivity for KAT2B over KAT2A. In some embodiments, DC₅₀ for KAT2B, e.g., when assessed in certain cells as in Example B1, is about or at least about 2, 3, 4, 5, 6, 7, 8, 9, 10, 15, 20, 30, 40, 50, 60, 70, 80, 90 or 100 fold more than DC₅₀ for KAT2A. In some embodiments, DC₅₀ for KAT2A, e.g., when assessed in certain cells as in Example B1, is about or at least about 2, 3, 4, 5, 6, 7, 8, 9, 10, 15, 20, 30, 40, 50, 60, 70, 80, 90 or 100 fold more than DC₅₀ for KAT2B. In some embodiments, certain compounds have DC₅₀ at about 1 nM or lower for both KAT2A and KAT2B. In some embodiments, certain compounds have DC₅₀ at about 1 nM or lower for both KAT2A and KAT2B, and have selectivity for KAT2A over KAT2B. In some embodiments, certain compounds have DC₅₀ at about 1 nM or lower for both KAT2A and KAT2B, and have selectivity for KAT2B over KAT2A. In some embodiments, certain compounds have DC₅₀ at about 1 nM or lower for KAT2A but DC₅₀ higher than about 1 nM for KAT2B. In some embodiments, certain compounds have DC₅₀ at about 1 nM or lower for KAT2B but DC₅₀ higher than about 1 nM for KAT2A. Certain examples are presented in Table 3 in the Examples.

In some embodiments, the present disclosure provides methods of administering provided compounds or compositions to a subject in need thereof. In some embodiments, the present disclosure provides methods of administering provided compounds or compositions to a subject suffering from or susceptible to a disease, disorder, or condition associated with KAT2 (e.g., KAT2A and/or KAT2B). In some embodiments, a condition, disorder or disease is associated with KAT2A. In some embodiments, a condition, disorder or disease is associated with level of KAT2A. In some embodiments, a condition, disorder or disease is associated with activity of KAT2A. In some embodiments, a condition, disorder or disease is associated with KAT2B. In some embodiments, a condition, disorder or disease is associated with level of KAT2B. In some embodiments, a condition, disorder or disease is associated with activity of KAT2B. In some embodiments, a condition, disorder or disease is associated with KAT2A and KAT2B. In some embodiments, a condition, disorder or disease is associated with level of KAT2A and KAT2B. In some embodiments, a condition, disorder or disease is associated with combined level of KAT2A and KAT2B. In some embodiments, a condition, disorder or disease is associated with activity of KAT2A and KAT2B.

In some embodiments, the present disclosure provides methods of treating a disease, disorder, or condition associated with KAT2 (e.g., KAT2A and/or KAT2B), comprising administering a provided compound or composition to a subject in need thereof. In some embodiments, the present disclosure provides methods of treating a disease, disorder, or condition associated with KAT2 (e.g., KAT2A and/or KAT2B), comprising administering or delivering a provided compound or composition to a subject in need thereof. In some embodiments, the present disclosure provides methods of treating a disease, disorder, or condition, comprising administering a provided compound or composition to a subject in need thereof. In some embodiments, the present disclosure provides methods of treating a disease, disorder, or condition, comprising delivering a provided compound or composition to a subject in need thereof. In some embodiments, provided methods are for treating cancer. In some embodiments, a cancer is characterized by a solid tumor. In some embodiments, a cancer is characterized by a neuroendocrine tumor. In some embodiments, a cancer is characterized by a hematologic tumor. In some embodiments, a cancer is selected from hematopoietic cancers, including leukemias, lymphomas (e.g., Hodgkin's and non-Hodgkin's), myelomas and myeloproliferative disorders; sarcomas, melanomas, adenomas, carcinomas of solid tissue, squamous cell carcinomas of the mouth, throat, larynx, and lung, liver cancer, genitourinary cancers such as prostate, cervical, bladder, uterine, and endometrial cancer and renal cell carcinomas, bone cancer, pancreatic cancer, skin cancer, cutaneous or intraocular melanoma, cancer of the endocrine system, cancer of the thyroid gland, cancer of the parathyroid gland, head and neck cancers, breast cancer, gastro-intestinal cancers and nervous system cancers, benign lesions such as papillomas, and the like. In some embodiments, provided methods are for treating a leukemia (e.g., acute myeloid leukemia, chronic myeloid leukemia, acute lymphocytic leukemia, or chronic lymphocytic leukemia.) In some embodiments, provided methods are for treating a disease, disorder, or condition selected from acute myeloid leukemia (AML), neuroblastoma, non-small cell lung cancer (NCSLC), small cell lung cancer (SCLC), colorectal cancer, melanoma, and prostate cancer. In some embodiments, a disease, disorder, or condition is acute myeloid leukemias (AML). In some embodiments, a disease, disorder, or condition is small cell lung cancer (SCLC). In some embodiments, a disease, disorder, or condition is neuroendocrine prostate cancer (NEPC). In some embodiments, a disease, disorder, or condition is high-grade neuroendocrine carcinomas, e.g., those in the GI tract and pancreas. In some embodiments, a disease, disorder, or condition is castration resistant prostate cancer (CRPC).

Phenotypic plasticity, or the ability of cancer cells to switch between cell states, has been recognized as a key hallmark of cancer. Cancer cells can hijack normal developmental pathways, preventing terminal differentiation or causing de-differentiation or trans-differentiation to a more proliferative, plastic cell state similar to those found in early development. Using its AI/ML platform AURIGIN, Applicant created a high-resolution atlas and identified histone acetyltransferase KAT2A as a key driver of tumor cell plasticity in various cancers such as certain acute myeloid leukemias (AML), small cell lung cancer (SCLC) and neuroendocrine prostate cancer (NEPC). Among other things, provided compounds are useful for treating such cancers. For example, compounds of the present disclosure were assessed across representative models of AML, SCLC and NEPC. In vitro, compounds of the present disclosure inhibited proliferation and promoted profound cell state changes in, e.g., MOLM-13 (AML) cells; in NCI-H1048 (SCLC), LASPC-01 (NEPC) cell lines as well as a primary NEPC organoid model, compounds of the present disclosure demonstrated potent antiproliferative effects; in vivo, compounds of the present disclosure significantly inhibited tumor growth in an NCI-H1048 xenograft model. Among other things, the present disclosure genetically and pharmacologically validated KAT2A and KAT2B as key drivers of cell state plasticity in tumors such as SCLC, NEPC and AML tumors and confirmed their utility as novel targets in these aggressive, metastatic and drug resistant cancers. Among other things, the present disclosure provides technologies, e.g., compounds, compositions, methods, etc., for treating such cancers.

In some embodiments, a provided compound or composition is administered as part of a combination therapy. As used herein, the term “combination therapy” refers to those situations in which a subject is simultaneously exposed to two or more therapeutic or prophylactic regimens (e.g., two or more therapeutic or prophylactic agents). In some embodiments, the two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, “administration” of combination therapy may involve administration of one or more agent(s) or modality(ies) to a subject receiving the other agent(s) or modality(ies) in the combination. For clarity, combination therapy does not require that individual agents be administered together in a single composition (or even necessarily at the same time), although in some embodiments, two or more agents, or active moieties thereof, may be administered together in a combination composition.

For example, in some embodiments, a provided compound or composition is administered to a subject who is receiving or has received one or more additional therapies (e.g., an anti-cancer therapy and/or therapy to address one or more side effects of such anti-cancer therapy, or otherwise to provide palliative care). The present disclosure incorporates various information, including patent application publications, patents and non-patent references, by reference; in the case of any conflict, the present disclosure will control.

As described herein, among other things, provided technologies can provide various benefits and/or advantages. For example, in some embodiments, provided technologies are more potent, e.g., when assessed for KAT2A and/or KAT2B degradation, cell state modulation, cell growth inhibition, tumor size reduction, and/or treatment of various conditions, diseases or disorders. In some embodiments, provided technologies provide improved bioavailability (e.g., oral bioavailability), flexibility in administration routes and/or regimens, reduced clearance (e.g., unbound clearance), increased exposure, increased low solubility, improved exposure levels, lower doses, etc. In some embodiments, properties, activities, parameters, etc., for assessment are cLogD, tPSA, molecular weight, kinetic solubility (e.g., at pH like about 7.4), dose (mg/kg, e.g., for achieving an effect or a result), CL (e.g., CL, u), T_{1/2} (e.g., in plasma, a system, etc.), Vdss, AUC (e.g., 0-last), mPPB (%), bioavailability, DC₅₀ (e.g., for KAT2A, KAT2B, etc.), DC₉₀, GI₅₀, GI₉₀, etc. In some embodiments, assessment is performed with cells, e.g., MOLM-13, NCI-H1048, LASCPC-01, etc. In some embodiments, assessment is performed with human primary cells (e.g., from a patent sample). In some embodiments, assessment is performed with a sample. In some embodiments, assessment is performed with a tissue. In some embodiments, assessment is performed with a organ. In some embodiments, assessment is performed with a organoid. In some embodiments, assessment is performed with an animal, e.g., an animal model.

In some embodiments, a reference technology for comparison is or comprises a reference compound or a salt thereof. In some embodiments, a reference compound is

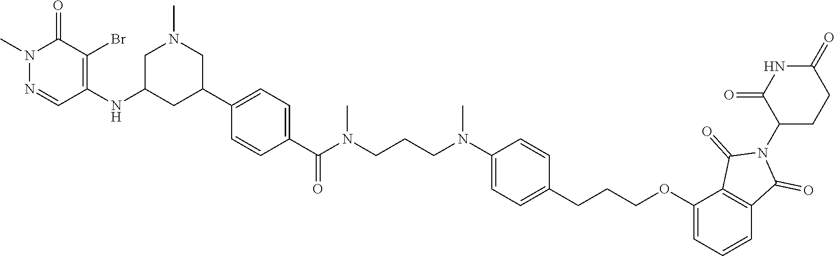 [SMILES: CN1CC(Nc2cnn(C)c(=O)c2Br)CC(c2ccc(C(=O)N(C)CCCN(C)c3ccc(CCCOc4cccc5c4C(=O)N(C4CCC(=O)NC4=O)C5=O)cc3)cc2)C1]

or a pharmaceutically acceptable salt thereof. In some embodiments, a reference compound is

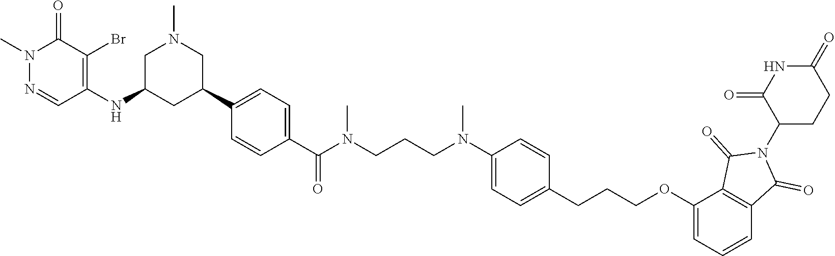 [SMILES: CN1C[C@H](Nc2cnn(C)c(=O)c2Br)C[C@H](c2ccc(C(=O)N(C)CCCN(C)c3ccc(CCCOc4cccc5c4C(=O)N(C4CCC(=O)NC4=O)C5=O)cc3)cc2)C1]

(Compound Ref-1) or a pharmaceutically acceptable salt thereof. In some embodiments, a reference compound is

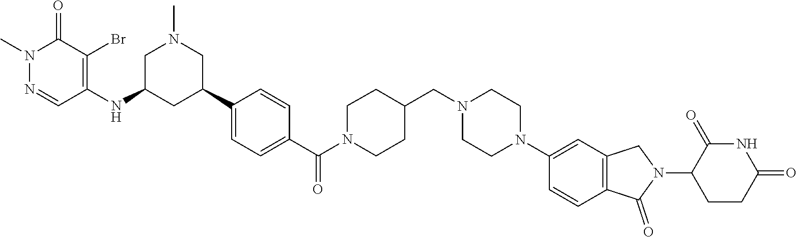 [SMILES: CN1C[C@H](Nc2cnn(C)c(=O)c2Br)C[C@H](c2ccc(C(=O)N3CCC(CN4CCN(c5ccc6c(c5)CN(C5CCC(=O)NC5=O)C6=O)CC4)CC3)cc2)C1]

or a pharmaceutically acceptable salt thereof.

The present disclosure incorporates various information, including patent application publications, patents and non-patent references, by reference; in the case of any conflict, the present disclosure will control.

Among other things, the present disclosure provides the following Embodiments as examples:
1. A compound of Formula I:

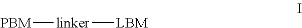

wherein:
   PBM is a KAT2 protein binding moiety;
   linker is an optional linking moiety; and
   LBM is an E3 ubiquitin ligase binding moiety.
2. A compound of Formula I:

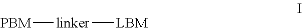

or a pharmaceutically acceptable salt thereof, wherein:
   PBM is a KAT2 protein binding moiety, or is

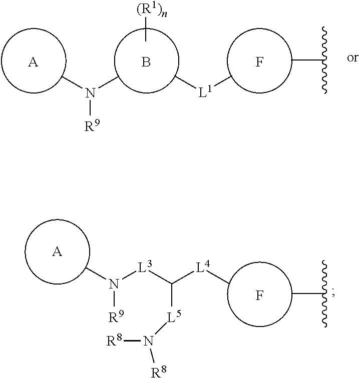

linker is an optional linking moiety, or is a covalent bond or an optionally substituted bivalent C₁-C₂₀ hydrocarbon chain, wherein one or more methylene units are optionally and independently replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —S(O)—, —SO₂—, —SO₂N(R)—, —N(R)SO₂—, or -Cy-; and
LBM is an E3 ubiquitin ligase binding moiety, or is

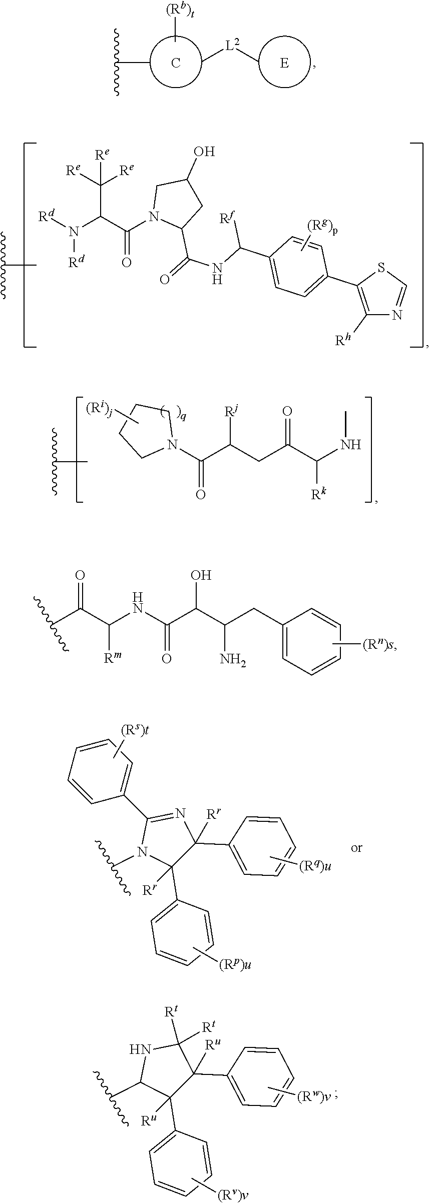

wherein:
Ring A is selected from:

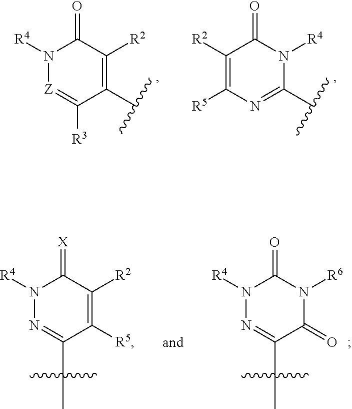

Ring B is an optionally substituted 5- to 6-membered heterocyclyl having 1-2 heteroatoms independently selected from N, O, and S;
L¹ is a covalent bond or an optionally substituted bivalent C₁₋₃ hydrocarbon chain;
each R¹ is independently —R;
n is 0, 1, 2, 3, or 4;
Z is N or CR³;
R² is halogen, —CN, or —R;
each R³ is independently halogen, —CN, or —R;
R⁴ is —R′;
R⁵ is halogen, —CN, or —R, or
   R² and R⁵, together with the atoms to which they are attached, combine to form an optionally substituted 5- to 6-membered ring having 0-2 heteroatoms independently selected from N, O, and S;
R⁶ is —R′;
X is O or NR⁷;
R⁷ is —R′, or
   R⁴ and R⁷, together with the atoms to which they are attached, combine to form an optionally substituted 5- to 6-membered ring having 2-3 heteroatoms independently selected from N, O, and S;
L³, L⁴, and L⁵ are each independently a covalent bond or an optionally substituted bivalent C₁₋₆ hydrocarbon chain;
each R⁸ is independently —R′;
R⁹ is —R′;
each Cy is independently an optionally substituted, 3- to 16-membered bivalent ring having 0-6 heteroatoms independently selected from N, O, and S;
each of Ring C and Ring F is independently an optionally substituted 3- to 16-membered ring having 0-6 heteroatoms independently selected from N, O, and S;
each R^{b} is independently halogen, —CN, —R′ or —OR;
t is 0, 1, 2, 3, or 4;
L² is a covalent bond or an optionally substituted C₁₋₃ hydrocarbon chain, wherein one or more methylene units are optionally and independently replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —S(O)—, —SO₂—, —SO₂N(R)—, —N(R)SO₂—, or -Cy-;
Ring E is

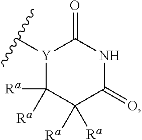

or an optionally substituted group selected from

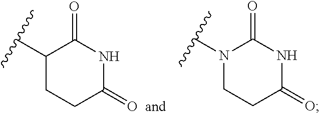

Y is N or CH;
each R^{a} is independently halogen, —CN or —R′;
linker is attached to the bracketed moiety at one of R^{d}, R^{e}, R^{f}, or R^{g}, or linker is attached to the bracketed moiety at one Rⁱ or the ring formed when two Rⁱ groups are taken together;
each R^{d} that is not the point of attachment for the linker is independently —R′, or
   two R^{d}, together with the atom to which they are attached, combine to form an optionally substituted 5- to 6-membered ring having 1-3 heteroatoms independently selected from N, O, and S and optionally fused to a phenyl or 5- to 6-membered heteroaryl ring having 1-3 heteroatoms independently selected from N, O, and S;
each R^{e} that is not the point of attachment for the linker is independently —R;
R^{f}, when it is not the point of attachment for the linker, is —R;
each R^{g} that is not the point of attachment for the linker is independently halogen, —CN, R or —OR;
p is 0, 1, 2, or 3;
Rh is halogen, or —R;
each Rⁱ that is not the point of attachment of the linker is independently halogen, —R, —C(O)N(R′)₂, or —N(R)C(O)R, or
   two Rⁱ groups, together with the atoms to which they are attached, combine to form an optionally substituted phenyl or 5- to 6-membered ring having 1-3 heteroatoms independently selected from N, O, and S;
R^{j} is —R, or
   R^{j} combines with one instance of Rⁱ, together with their intervening atoms, to form an optionally substituted 5- to 7-membered ring having 1-2 heteroatoms independently selected from N, O, and S;
R^{k} is —R;
r is 1, 2, 3, 4, or 5;
q is 1 or 2;
R^{m} is —R;
each of Rⁿ, R^{p}, R^{q}, R^{r}, R^{s}, and R^{u} is independently halogen, —CN, —R or —OR;
s is 0, 1, 2, 3, 4, or 5;
t is 0, 1, 2, 3, 4, or 5;
each u is independently 0, 1, 2, 3, 4, or 5;
each R^{t} is independently R, or
   both R^{t} groups, together with the atom to which they are attached, combine to form an optionally substituted 3- to 7-membered ring having 0-2 heteroatoms independently selected from N, O, and S;
each R^{v} is independently halogen, —CN, —R or —OR, or
   one instance of R^{u} and R^{v}, together with their intervening atoms, combine to form an optionally substituted 3- to 7-membered ring having 0-2 heteroatoms independently selected from N, O, and S;
each R^{w} is independently halogen, —CN, —R or —OR, or
   one instance of R^{u} and R^{w}, together with their intervening atoms, combine to form an optionally substituted 3- to 7-membered ring having 0-2 heteroatoms independently selected from N, O, and S;
each v is independently 0, 1, 2, 3, 4, or 5;
each R′ is independently —R, —C(O)R, or —S(O)₂R, or two R′ attached to the same atom, together with the atom to which they are attached, combine to form an optionally substituted 3- to 16-membered ring having 1-5 heteroatoms independently selected from N, O, and S; and
each R is independently hydrogen or an optionally substituted group selected from C₁₋₈ aliphatic, C₃₋₁₀ cycloaliphatic, C₁-C₈ heteroaliphatic having 1-3 heteroatoms independently selected from N, O, and S, C₆₋₁₀ aryl, 5- to 10-membered heteroaryl having 1-5 heteroatoms independently selected from N, O, and S, and 3- to 10-membered monocyclic heterocyclyl having 1-5 heteroatoms independently selected from N, O, and S.
3. The compound of any one of the preceding Embodiments, wherein the PBM is a KAT2A protein binding moiety.
4. The compound of any one of the preceding Embodiments, wherein the PBM is a KAT2B protein binding moiety.
5. The compound of any one of the preceding Embodiments, wherein PBM is

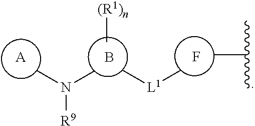

6. A compound of Formula II-1:

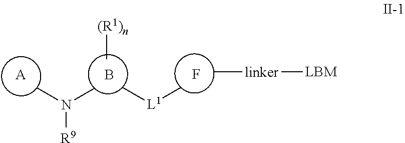

or a pharmaceutically acceptable salt thereof, wherein:
   Ring A is selected from:

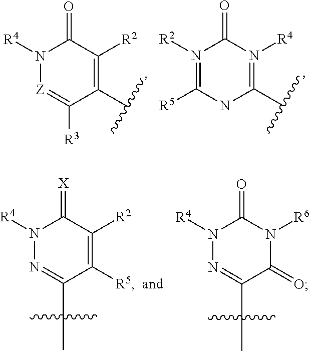

Ring B is an optionally substituted 5- to 6-membered heterocyclyl having 1-2 heteroatoms independently selected from N, O, and S;
L¹ is a covalent bond or an optionally substituted bivalent C₁₋₃ hydrocarbon chain;
each R¹ is independently —R;
n is 0, 1, 2, 3, or 4;
Z is N or CR³;
R² is halogen, —CN, or —R;
each R³ is independently halogen, —CN, or —R;
R⁴ is —R′;
R⁵ is halogen, —CN, or —R, or
   R² and R⁵, together with the atoms to which they are attached, combine to form an optionally substituted 5- to 6-membered ring having 0-2 heteroatoms independently selected from N, O, and S;
R⁶ is —R′;
X is O or NR⁷;
R⁷ is —R′, or
   R⁴ and R⁷, together with the atoms to which they are attached, combine to form an optionally substituted 5- to 6-membered ring having 2-3 heteroatoms independently selected from N, O, and S;
R⁹ is —R′;
linker is a covalent bond or an optionally substituted bivalent C₁-C₂₀ hydrocarbon chain, wherein one or more methylene units are optionally and independently replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —S(O)—, —SO₂—, —SO₂N(R)—, —N(R)SO₂—, or -Cy-;
each Cy is independently an optionally substituted, 3- to 16-membered bivalent ring system having 0-6 heteroatoms independently selected from N, O, and S;
Ring F is an optionally substituted 3- to 16-membered ring having 0-6 heteroatoms independently selected from N, O, and S;
LBM is an E3 ubiquitin ligase binding moiety;
each R′ is independently —R, —C(O)R, or —S(O)₂R, or two R′ attached to the same atom, together with the atom to which they are attached, combine to form an optionally substituted 3-16 membered ring having 1-5 heteroatoms independently selected from N, O, and S;
each R is independently hydrogen or an optionally substituted group selected from C₁₋₈ aliphatic, C₆₋₁₀ aryl, C₃₋₁₀ cycloaliphatic, 5- to 10-membered heteroaryl having 1-5 heteroatoms independently selected from N, O, and S, and a 3- to 10-membered monocyclic heterocyclyl having 1-5 heteroatoms independently selected from N, O, and S.
7. The compound of any one of Embodiments 2-6, wherein Ring F is an optionally substituted 3- to 10-membered ring having 0-4 heteroatoms independently selected from N, O, and S.
8. The compound of any one of Embodiments 2-6, wherein Ring F is an optionally substituted 5-6 membered ring having 0-4 heteroatoms independently selected from N, O, and S.
9. The compound of any one of Embodiments 2-8, wherein Ring F is an optionally substituted 6-membered ring.
10. The compound of any one of Embodiments 2-9, wherein Ring F is aromatic.
11. The compound of any one of Embodiments 2-10, wherein Ring F is an optionally substituted phenyl ring.
12. The compound of any one of Embodiments 2-10, wherein Ring F is optionally substituted 1,4-phenylene.
13. The compound of any one of Embodiments 2-10, wherein Ring F is 1,4-phenylene.
14. A compound of Formula II:

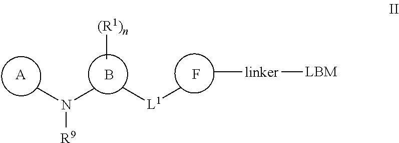

or a pharmaceutically acceptable salt thereof, wherein:
   Ring A is selected from:

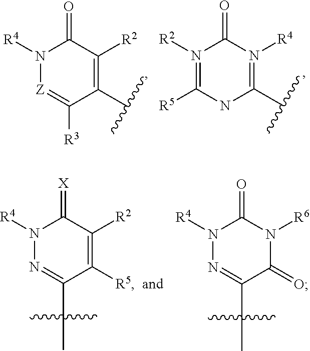

Ring B is a 5- to 6-membered heterocyclyl having 1-2 heteroatoms independently selected from N, O, and S;
L¹ is a covalent bond or a bivalent C₁₋₃ straight or branched hydrocarbon chain;
each R¹ is independently optionally substituted C₁₋₆ aliphatic, or optionally substituted C₃₋₆ cycloaliphatic;
n is 0, 1, 2, 3, or 4;
Z is N or CR³;
R² is hydrogen, halogen, —CN, optionally substituted C₁₋₆ aliphatic, or optionally substituted C₃₋₆ cycloaliphatic;
each R³ is independently hydrogen, halogen, optionally substituted C₁₋₆ aliphatic, or optionally substituted C₃₋₆ cycloaliphatic;
R⁴ is hydrogen, optionally substituted C₁₋₆ aliphatic, or optionally substituted C₃₋₆ cycloaliphatic;
R⁵ is hydrogen, halogen, optionally substituted C₁₋₆ aliphatic, or optionally substituted C₃₋₆ cycloaliphatic, or
   R² and R⁵, together with the atoms to which they are attached, combine to form an optionally substituted 5- to 6-membered aromatic ring having 0-2 heteroatoms independently selected from N, O, and S;
R⁶ is hydrogen, optionally substituted C₁₋₆ aliphatic, or optionally substituted C₃₋₆ cycloaliphatic;
X is O or NR⁷;
R⁷ is hydrogen or optionally substituted C₁₋₆ aliphatic, or
   R⁴ and R⁷, together with the atoms to which they are attached, combine to form an optionally substituted 5- to 6-membered aromatic ring having 2-3 heteroatoms independently selected from N, O, and S;
R⁹ is hydrogen or optionally substituted C₁₋₆ aliphatic;
linker is a covalent bond or an optionally substituted, bivalent, straight or branched, saturated or unsaturated C₁-C₂₀ hydrocarbon chain, wherein one or more methylene units are optionally and independently replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —SO₂, —SO₂N(R)—, —N(R)SO₂—, or -Cy-;
each Cy is independently an optionally substituted, mono- or multicyclic, 3- to 16-membered bivalent ring system, wherein the ring system is fully saturated, partially saturated, or aromatic, and the ring system contains 0-6 heteroatoms independently selected from N, O, and S;
each R is independently hydrogen or an optionally substituted group selected from C₁₋₆ aliphatic, phenyl, C₃₋₇ cycloaliphatic, 5- to 6-membered monocyclic heteroaryl having 1-4 heteroatoms independently selected from N, O, and S, or a 3- to 7-membered monocyclic heterocyclyl having 1-2 heteroatoms independently selected from N, O, and S; and
LBM is an E3 ubiquitin ligase binding moiety.
15. The compound of any one of Embodiments 2-14, wherein R² is hydrogen, halogen, optionally substituted C₁₋₆ aliphatic, or optionally substituted C₃₋₆ cycloaliphatic.
16. The compound of any one of Embodiments 2-14, wherein R² is hydrogen.
17. The compound of any one of Embodiments 2-14, wherein R² is halogen.
18. The compound of any one of Embodiments 2-14, wherein R² is —Cl.
19. The compound of any one of Embodiments 2-14, wherein R² is —Br.
20. The compound of any one of Embodiments 2-14, wherein R² is optionally substituted C₁₋₆ aliphatic, or optionally substituted C₃₋₆ cycloaliphatic.
21. The compound of any one of Embodiments 2-14, wherein R³ is hydrogen.
22. The compound of any one of Embodiments 2-21, wherein Ring A is

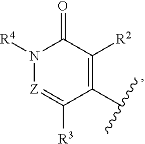

23. The compound of Embodiment 22, wherein Z is N.
24. The compound of Embodiment 22, wherein Z is CR³.
25. The compound of any one of Embodiments 22-24, wherein R⁴ is optionally substituted C₁₋₆ aliphatic.
26. The compound of any one of Embodiments 22-24, wherein R⁴ is optionally substituted C₁₋₆ alkyl.
27. The compound of any one of Embodiments 22-24, wherein R⁴ is C₁₋₆ alkyl.
28. The compound of any one of Embodiments 22-24, wherein R⁴ is methyl.
29. The compound of Embodiment 22, wherein Ring A is selected from

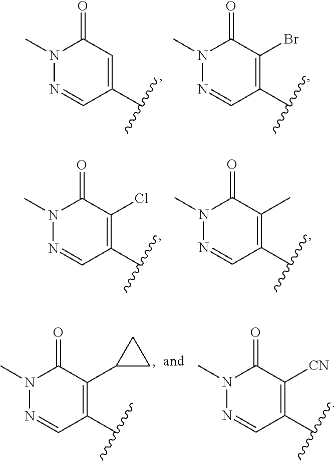

30. The compound of Embodiment 22, wherein Ring A is selected from

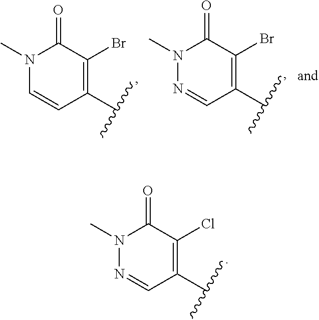

31. The compound of Embodiment 22, wherein Ring A is

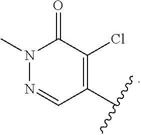 [SMILES: *c1cnn(C)c(=O)c1Cl]

32. The compound of Embodiment 22, wherein Ring A is

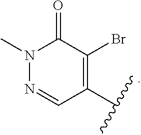 [SMILES: *c1cnn(C)c(=O)c1Br]

33. The compound of any one of Embodiments 2-21, wherein Ring A is

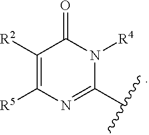

34. The compound of any one of Embodiments 2-21, wherein Ring A is

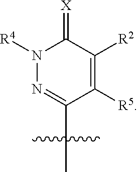

35. The compound of Embodiment 34, wherein X is O.
36. The compound of Embodiment 34, wherein X is NR⁷.
37. The compound of Embodiment 36, wherein R⁴ and R⁷, together with the atoms to which they are attached, combine to form an optionally substituted 5- to 6-membered ring having 2-3 heteroatoms independently selected from N, O, and S.
38. The compound of Embodiment 37, wherein the formed ring is aromatic.
39. The compound of any one of Embodiments 33-38, wherein R² and R⁵, together with the atoms to which they are attached, combine to form an optionally substituted 5- to 6-membered ring having 0-2 heteroatoms independently selected from N, O, and S.
40. The compound of Embodiment 39, wherein the formed ring is aromatic.
41. The compound of any one of Embodiments 2-21, wherein Ring A is

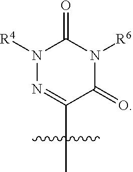

42. The compound of Embodiment 41, wherein R⁶ is optionally substituted C₁₋₆ aliphatic
43. The compound of any one of Embodiments 33-42, wherein R⁴ is optionally substituted C₁₋₆ aliphatic.
44. The compound of any one of Embodiments 33-42, wherein R⁴ is optionally substituted C₁₋₆ alkyl.
45. The compound of any one of Embodiments 33-42, wherein R⁴ is C₁₋₆ alkyl.
46. The compound of any one of Embodiments 33-42, wherein R⁴ is methyl,
47. The compound of Embodiment 33, wherein Ring A is

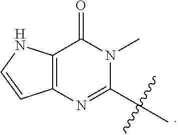 [SMILES: *c1nc2cc[nH]c2c(=O)n1C]

48. The compound of Embodiment 34, wherein Ring A is selected from

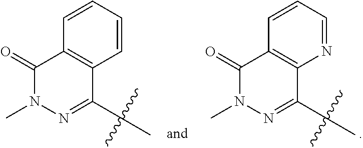 [SMILES: *c1nn(C)c(=O)c2ccccc12 ; *c1nn(C)c(=O)c2cccnc12]

49. The compound of Embodiment 34, wherein Ring A is

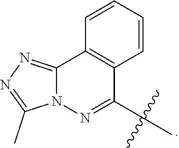 [SMILES: *c1nn2c(C)nnc2c2ccccc12]

50. The compound of Embodiment 41, wherein Ring A is

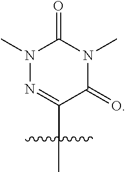

51. The compound of any one of Embodiments 2-50, wherein R⁹ is hydrogen.
52. The compound of any one of Embodiments 2-51, wherein Ring B is saturated.
53. The compound of any one of Embodiments 2-52, wherein Ring B is monocyclic.
54. The compound of any one of Embodiments 2-53, wherein Ring B is an optionally substituted 6-membered heterocyclyl having 1-2 heteroatoms independently selected from N, O, and S.
55. The compound of any one of Embodiments 2-53, wherein Ring B is optionally substituted

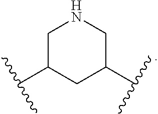 [SMILES: *C1CNCC(*)C1]

56. The compound of any one of Embodiments 2-55, wherein

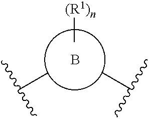

is

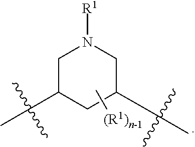

57. The compound of any one of Embodiments 2-56, wherein

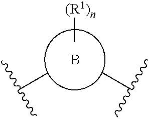

is

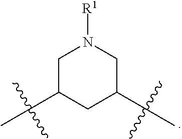

58. The compound of any one of Embodiments 2-57, wherein R¹ is optionally substituted C₁₋₆ aliphatic.
59. The compound of any one of Embodiments 2-58, wherein each R¹ is independently optionally substituted C₁₋₆ alkyl.
60. The compound of any one of Embodiments 2-58, wherein each R¹ is independently C₁₋₆ alkyl.
61. The compound of any one of Embodiments 2-58, wherein R¹ is methyl.
62. The compound of any one of Embodiments 2-61, wherein L¹ is a covalent bond.
63. The compound of any one of Embodiments 2-55, wherein n is 0 or 1.
64. The compound of any one of Embodiments 2-63, wherein

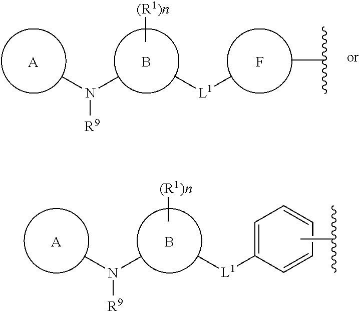

is selected from

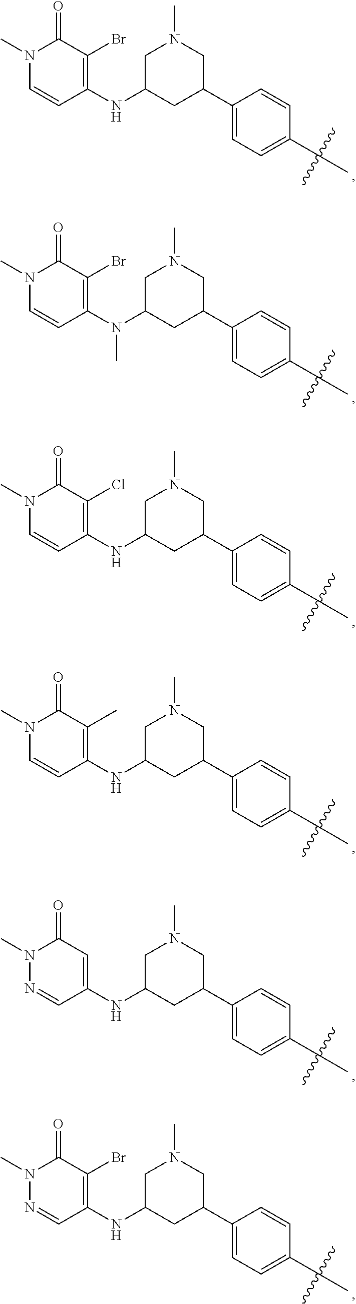 [SMILES: *c1ccc(C2CC(Nc3ccn(C)c(=O)c3Br)CN(C)C2)cc1 ; *c1ccc(C2CC(N(C)c3ccn(C)c(=O)c3Br)CN(C)C2)cc1 ; *c1ccc(C2CC(Nc3ccn(C)c(=O)c3Cl)CN(C)C2)cc1 ; *c1ccc(C2CC(Nc3ccn(C)c(=O)c3C)CN(C)C2)cc1 ; *c1ccc(C2CC(Nc3cnn(C)c(=O)c3)CN(C)C2)cc1 ; *c1ccc(C2CC(Nc3cnn(C)c(=O)c3Br)CN(C)C2)cc1]

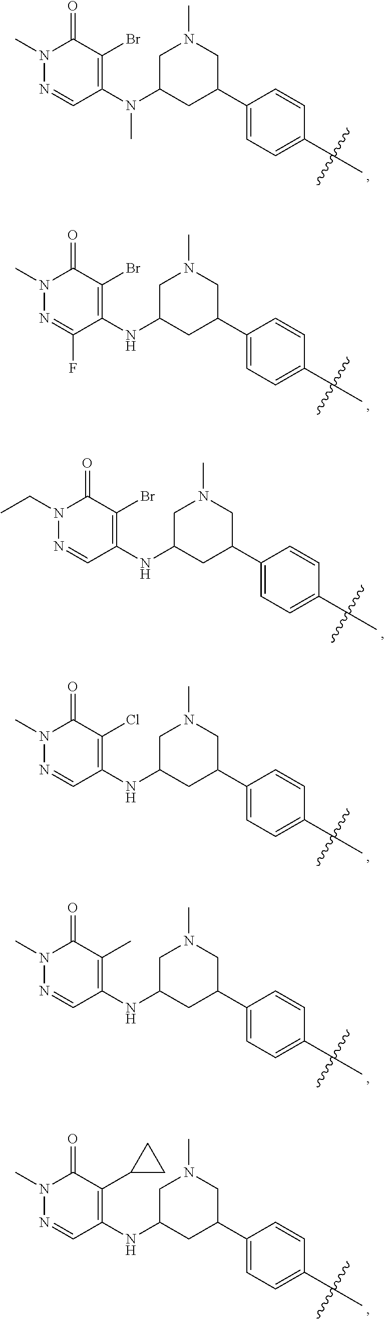 [SMILES: *c1ccc(C2CC(N(C)c3cnn(C)c(=O)c3Br)CN(C)C2)cc1 ; *c1ccc(C2CC(Nc3c(F)nn(C)c(=O)c3Br)CN(C)C2)cc1 ; *c1ccc(C2CC(Nc3cnn(CC)c(=O)c3Br)CN(C)C2)cc1 ; *c1ccc(C2CC(Nc3cnn(C)c(=O)c3Cl)CN(C)C2)cc1 ; *c1ccc(C2CC(Nc3cnn(C)c(=O)c3C)CN(C)C2)cc1 ; *c1ccc(C2CC(Nc3cnn(C)c(=O)c3C3CC3)CN(C)C2)cc1]

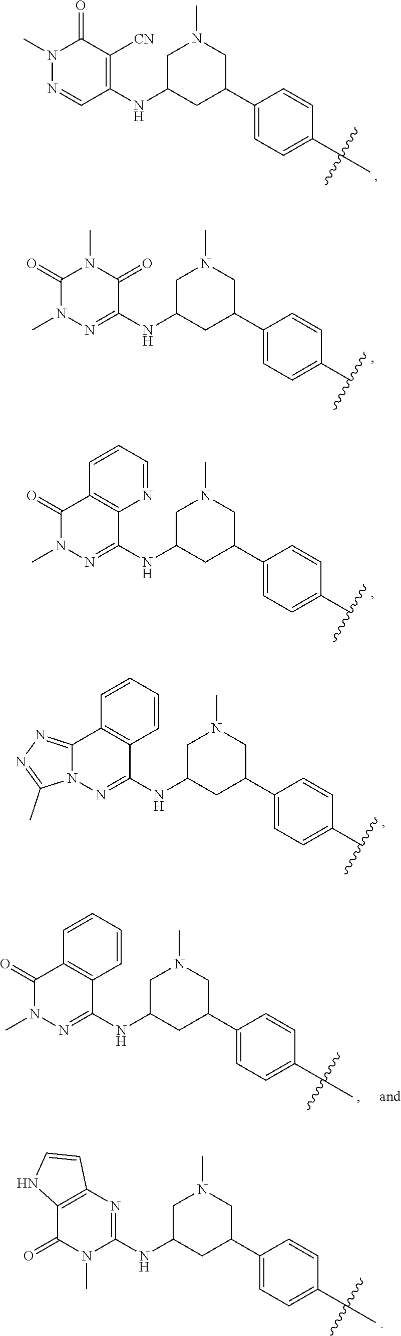 [SMILES: *c1ccc(C2CC(Nc3cnn(C)c(=O)c3C#N)CN(C)C2)cc1 ; *c1ccc(C2CC(Nc3nn(C)c(=O)n(C)c3=O)CN(C)C2)cc1 ; *c1ccc(C2CC(Nc3nn(C)c(=O)c4cccnc34)CN(C)C2)cc1 ; *c1ccc(C2CC(Nc3nn4c(C)nnc4c4ccccc34)CN(C)C2)cc1 ; *c1ccc(C2CC(Nc3nn(C)c(=O)c4ccccc34)CN(C)C2)cc1 ; *c1ccc(C2CC(Nc3nc4cc[nH]c4c(=O)n3C)CN(C)C2)cc1]

65. The compound of any one of Embodiments 2-63, wherein

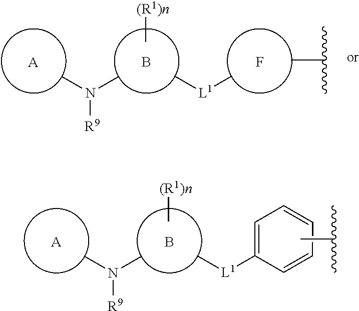

is selected from

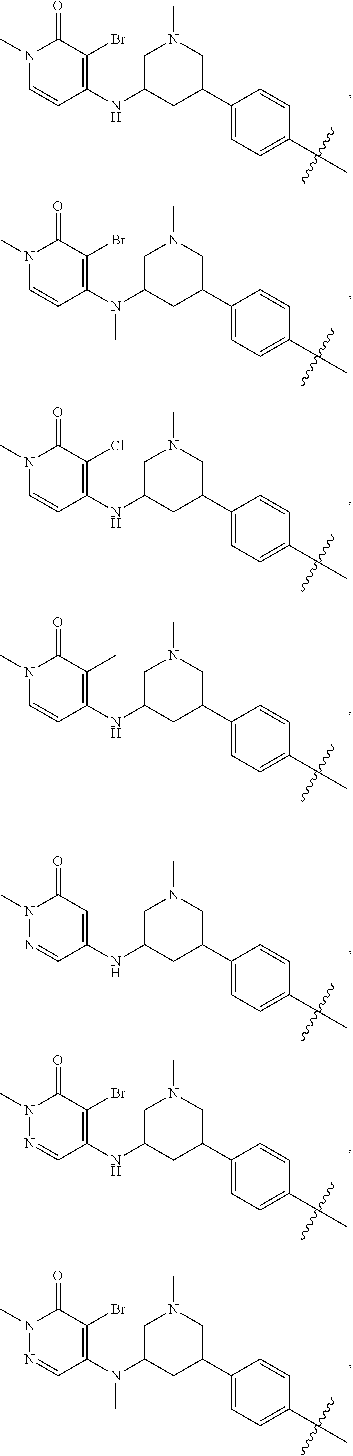

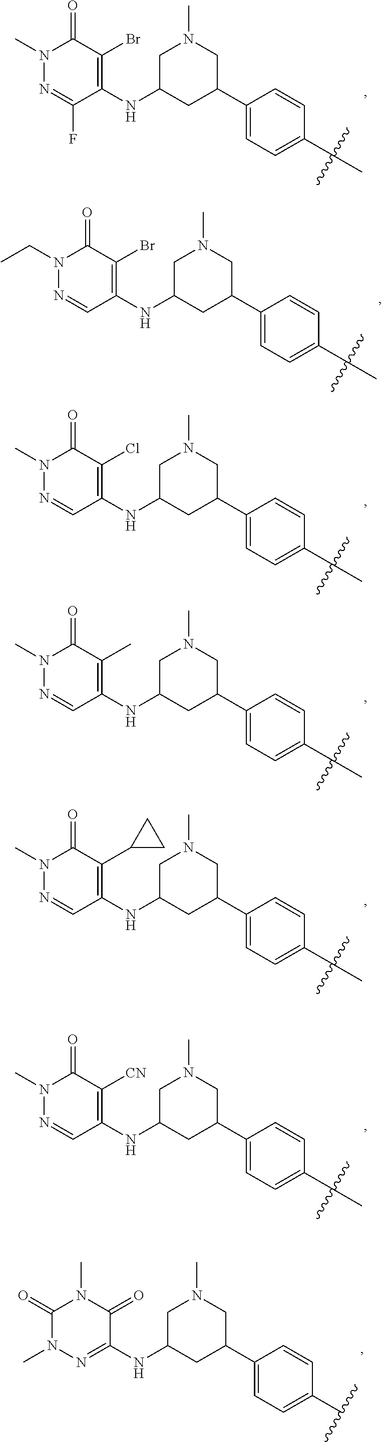

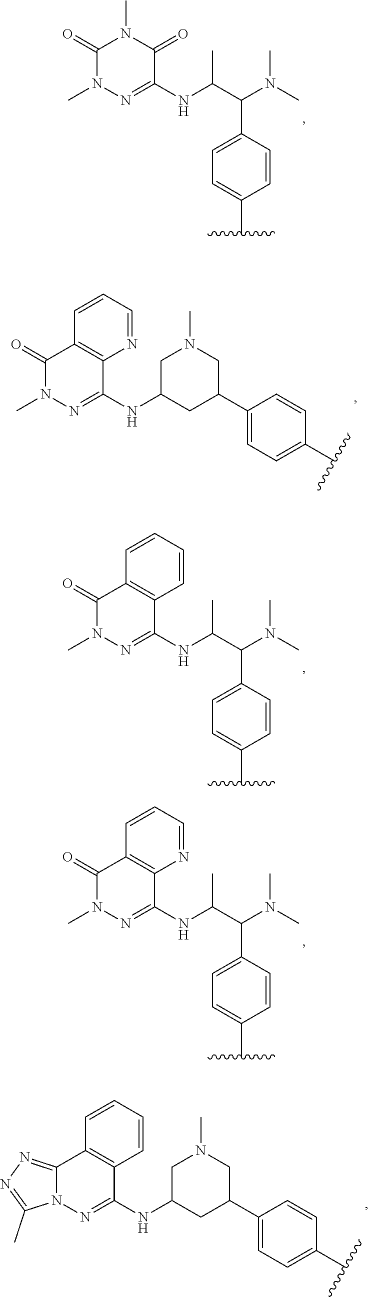 [SMILES: *c1ccc(C(C(C)Nc2nn(C)c(=O)n(C)c2=O)N(C)C)cc1 ; *c1ccc(C2CC(Nc3nn(C)c(=O)c4cccnc34)CN(C)C2)cc1 ; *c1ccc(C(C(C)Nc2nn(C)c(=O)c3ccccc23)N(C)C)cc1 ; *c1ccc(C(C(C)Nc2nn(C)c(=O)c3cccnc23)N(C)C)cc1 ; *c1ccc(C2CC(Nc3nn4c(C)nnc4c4ccccc34)CN(C)C2)cc1]

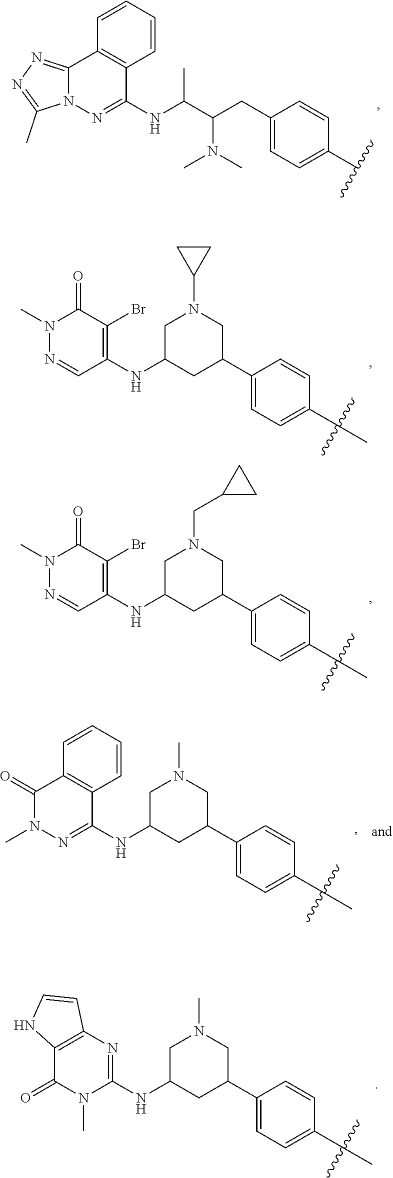 [SMILES: *c1ccc(CC(C(C)Nc2nn3c(C)nnc3c3ccccc23)N(C)C)cc1 ; *c1ccc(C2CC(Nc3cnn(C)c(=O)c3Br)CN(C3CC3)C2)cc1 ; *c1ccc(C2CC(Nc3cnn(C)c(=O)c3Br)CN(CC3CC3)C2)cc1 ; *c1ccc(C2CC(Nc3nn(C)c(=O)c4ccccc34)CN(C)C2)cc1 ; *c1ccc(C2CC(Nc3nc4cc[nH]c4c(=O)n3C)CN(C)C2)cc1]

66. The compound of any one of the preceding Embodiments, wherein the linker is less than 14 atoms in length.
67. The compound of any one of the preceding Embodiments, wherein the linker is less than 11 atoms in length.
68. The compound of any one of the preceding Embodiments, wherein the linker is less than 11 atoms in length.
69. The compound of any one of the preceding Embodiments, wherein the shortest-path length of linker is about or no more than 14 atoms.
70. The compound of any one of the preceding Embodiments, wherein the shortest-path length of linker is about or no more than 10 atoms.
71. The compound of any one of the preceding Embodiments, wherein the number of non-ring consecutive sp³ atoms in the shortest path chain of linker is about or no more than about 5, 4, 3, 2 or 1.
72. The compound of any one of the preceding Embodiments, wherein the number of non-ring consecutive sp³ atoms in the shortest path chain of linker is about or no more than about 2.
73. The compound of any one of the preceding Embodiments, wherein the number of bonds between two non-ring sp³ atoms in the shortest path chain of linker is about or no more than 5, 4, 3, 2 or 1.
74. The compound of any one of the preceding Embodiments, wherein the number of bonds between two non-ring sp³ atoms in the shortest path chain of linker is about or no more than 1.
75. The compound of any one of the preceding Embodiments, wherein the number of non-ring sp³ C, O, and S atoms in the shortest path chain of linker is about or no more than about 5, 4, 3, 2 or 1.
76. The compound of any one of the preceding Embodiments, wherein the number of non-ring sp³ C, O, and S atoms in the shortest path chain of linker is about or no more than about 1.
77. The compound of any one of the preceding Embodiments, wherein the number of non-ring sp³ atoms in the shortest path chain of linker is about or no more than about 5, 4, 3, 2 or 1.
78. The compound of any one of the preceding Embodiments, wherein the number of non-ring sp³ atoms in the shortest path chain of linker is about or no more than about 1.
79. The compound of any one of the preceding Embodiments, wherein the number of non-ring atoms in the shortest path chain of linker is about or no more than about 5, 4, 3, 2 or 1.
80. The compound of any one of the preceding Embodiments, wherein the number of non-ring atoms in the shortest path chain of linker is about or no more than about 1.
81. The compound of any one of the preceding Embodiments, wherein linker is an optionally substituted, bivalent, straight or branched, saturated or unsaturated C₁-C₂₀ hydrocarbon chain, wherein one or more methylene units are optionally and independently replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —SO₂—, —SO₂N(R)—, —N(R)SO₂—, or -Cy-.
82. The compound of any one of the preceding Embodiments, wherein linker is an optionally substituted, bivalent, straight or branched, saturated or unsaturated C₁-C₁₀ hydrocarbon chain, wherein one or more methylene units are optionally and independently replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —SO₂—, —SO₂N(R)—, —N(R)SO₂—, or -Cy-.
83. The compound of any one of the preceding Embodiments, wherein linker is an optionally substituted, bivalent, straight or branched, saturated or unsaturated C₁-C₂₀ hydrocarbon chain, wherein at least one methylene unit is replaced by —O—, —N(R)—, —C(O)—, —OC(O)—, —C(O)O—, —C(O)N(R)—, —N(R)C(O)—, or -Cy-.
84. The compound of any one of the preceding Embodiments, wherein linker is an optionally substituted, bivalent, straight or branched, saturated or unsaturated C₁-C₂₀ hydrocarbon chain, wherein at least two methylene units are replaced by —O—, —N(R)—, —C(O)—, —OC(O)—, —C(O)O—, —C(O)N(R)—, —N(R)C(O)—, or -Cy-.
85. The compound of any one of the preceding Embodiments, wherein a methylene unit is replaced by —C(O)—.
86. The compound of any one of the preceding Embodiments, wherein a methylene unit is replaced by -Cy-.
87. The compound of Embodiment 85, wherein -Cy- is in a shortest path chain.
88. The compound of any one of the preceding Embodiments, wherein linker is an optionally substituted, bivalent, straight or branched, saturated or unsaturated C₁-C₂₀ hydrocarbon chain, wherein at least one methylene unit is replaced by —C(O)N(R)—.
89. The compound of any one of the preceding Embodiments, wherein linker comprises at least one triple bond.
90. The compound of any one of the preceding Embodiments, wherein linker is an optionally substituted, bivalent, straight or branched, saturated or unsaturated C₁-C₂₀ hydrocarbon chain, wherein at least one methylene unit is replaced by -Cy-.
91. The compound of any one of the preceding Embodiments, wherein linker is:

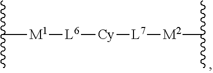

wherein:
   M¹ and M² are each independently absent, —CH₂—, —O—, —N(R)—, —C(O)—, —OC(O)—, —C(O)O—, —C(O)N(R)—, or —N(R)C(O)—; and
   L⁶ and L⁷ are each independently a covalent bond or an optionally substituted, bivalent, straight or branched, saturated or unsaturated C₁-C₁₀ hydrocarbon chain, wherein one or more methylene units are optionally and independently replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —SO₂—, —SO₂N(R)—, —N(R)SO₂—, or -Cy-.
92. The compound of any one of the preceding Embodiments, wherein linker is selected from

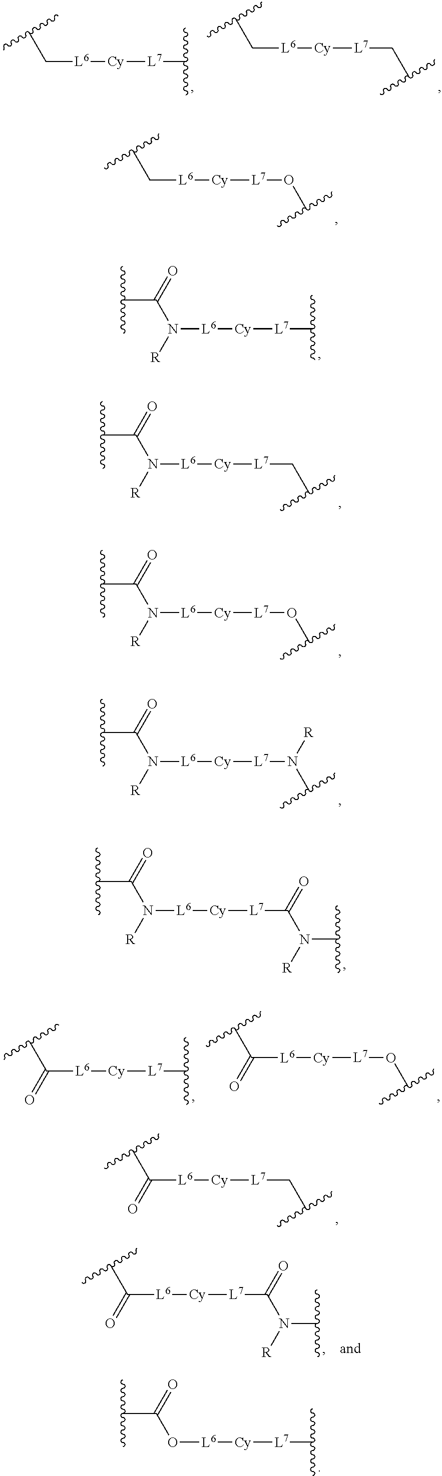

93. The compound of any one of the preceding Embodiments, wherein -Cy- is monocyclic.
94. The compound of Embodiment 92, wherein -Cy- is 3-10 membered.
95. The compound of any one of Embodiments 92-93, wherein -Cy- is 4-, 5- or 6-membered.
96. The compound of any one of Embodiments 92-94, wherein -Cy- is saturated.
97. The compound of any one of Embodiments 92-94, wherein -Cy- is partially unsaturated.
98. The compound of any one of Embodiments 93-97, wherein -Cy- has 1-2 heteroatoms independently selected from N, O, and S.
99. The compound of any one of Embodiments 93-98, wherein -Cy- has at least one nitrogen atom.
100. The compound of any one of Embodiments 1-92, wherein -Cy- is polycyclic.
101. The compound of any one of Embodiments 1-100, wherein -Cy- is bicyclic.
102. The compound of any one of Embodiments 100-101, wherein -Cy- is 6-16 membered.
103. The compound of any one of Embodiments 100-102, wherein -Cy- is 7-12 membered.
104. The compound of any one of Embodiments 100-103, wherein -Cy- is 7-, 8-, 9-, 10- or 11-membered.
105. The compound of any one of Embodiments 100-103, wherein -Cy- is 11-membered.
106. The compound of any one of Embodiments 100-105, wherein each monocyclic ring unit is independently 4-7 membered.
107. The compound of any one of Embodiments 100-106, wherein each monocyclic ring unit is independently 4-6 membered.
108. The compound of any one of Embodiments 100-107, wherein a monocyclic ring unit is saturated.
109. The compound of any one of Embodiments 100-108, wherein a monocyclic ring unit has 1-2 heteroatoms independently selected from N, O, and S.
110. The compound of any one of Embodiments 100-109, wherein each monocyclic ring unit independently has 1-2 heteroatoms independently selected from N, O, and S.
111. The compound of any one of Embodiments 100-110, wherein each monocyclic ring unit independently has at least one nitrogen atom.
112. The compound of any one of Embodiments 100-111, wherein -Cy- is saturated.
113. The compound of any one of Embodiments 100-111, wherein a monocyclic ring unit is partially unsaturated.
114. The compound of any one of Embodiments 100-113, wherein -Cy- is spiro.
115. The compound of any one of the preceding Embodiments, wherein linker comprises one or more basic nitrogen.
116. The compound of any one of the preceding Embodiments, wherein -Cy- comprises one or more basic nitrogen.
117. The compound of any one of the preceding Embodiments, wherein -Cy- is a diamine ring.
118. The compound of any one of Embodiments 2-92, wherein Cy is optionally substituted phenyl or 5- to 6-membered heteroaryl having 1-4 heteroatoms independently selected from N, O, and S.
119. The compound of any one of Embodiments 2-92, wherein Cy is optionally substituted monocyclic 5- to 6-membered heterocyclyl having 1-2 heteroatoms independently selected from N, O, and S.
120. The compound of any one of Embodiments 2-92, wherein Cy is optionally substituted bicyclic 6- to 11-membered heterocyclyl having 1-3 heteroatoms independently selected from N, O, and S.
121. The compound of any one of Embodiments 2-92, wherein Cy is a spirocyclic 6- to 7-membered heterocyclyl having 1-3 heteroatoms independently selected from N, O, and S.
122. The compound of any one of the preceding Embodiments, wherein -Cy- is substituted.
123. The compound of any one of the preceding Embodiments, wherein -Cy- is unsubstituted.
124. The compound of any one of Embodiments 2-91, wherein linker is selected from

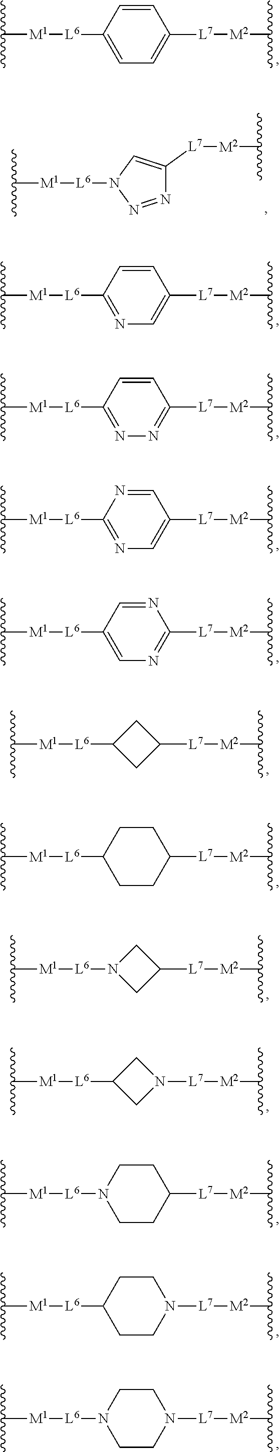

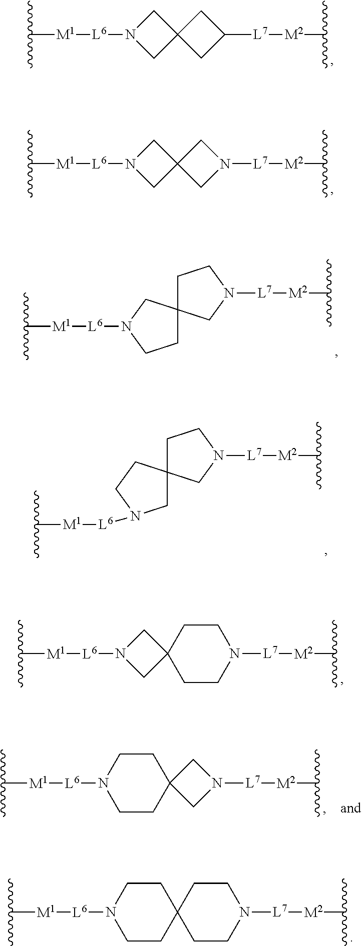

125. The compound of any one of Embodiments 1-90, wherein linker is -M¹-L⁶-L⁷-M²-.
126. The compound of any one of Embodiments 91-125, wherein M¹ is a covalent bond.
127. The compound of any one of Embodiments 91-125, wherein M¹ is optionally substituted —CH₂—.
128. The compound of any one of Embodiments 91-125, wherein M¹ is —CH₂—.
129. The compound of any one of Embodiments 91-125, wherein M¹ is —C(O)—.
130. The compound of any one of Embodiments 91-129, wherein M¹ is bonded to PBM.
131. The compound of any one of Embodiments 91-130, wherein L⁶ is a covalent bond.
132. The compound of any one of Embodiments 91-130, wherein L⁶ is optionally substituted —CH₂—.
133. The compound of any one of Embodiments 91-132, wherein L⁷ is a covalent bond.
134. The compound of any one of Embodiments 91-132, wherein L⁷ is —O—.
135. The compound of any one of Embodiments 91-134, wherein M² is a covalent bond.
136. The compound of any one of Embodiments 91-134, wherein M² is —O—.
137. The compound of any one of the preceding Embodiments, wherein each R is independently hydrogen or optionally substituted C₁₋₆ alkyl.
138. The compound of any one of Embodiments 1-65, wherein the linker is selected from: —CH₂—.

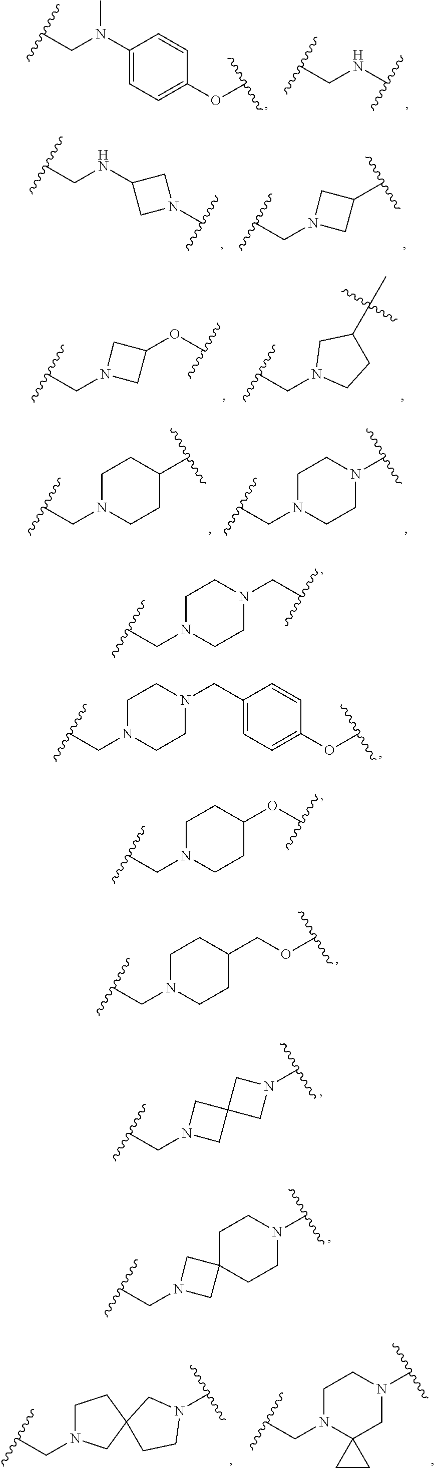

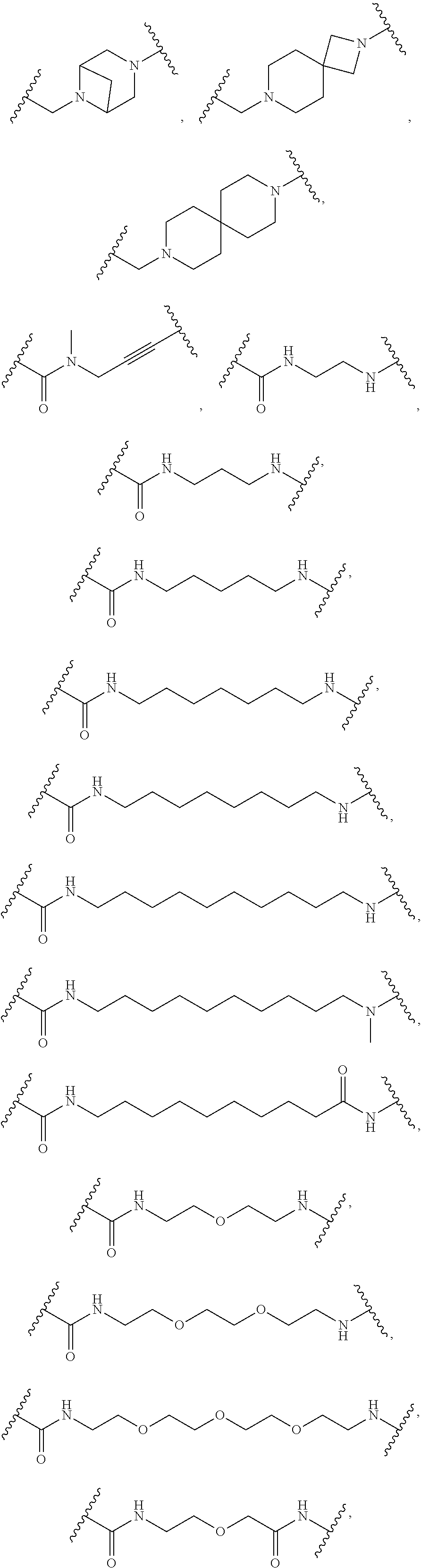

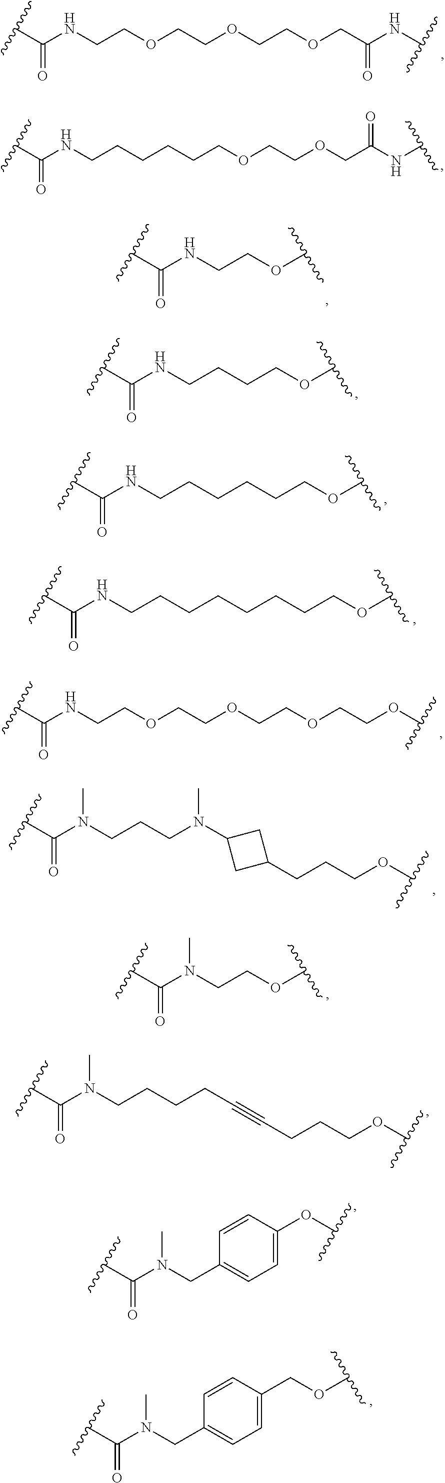

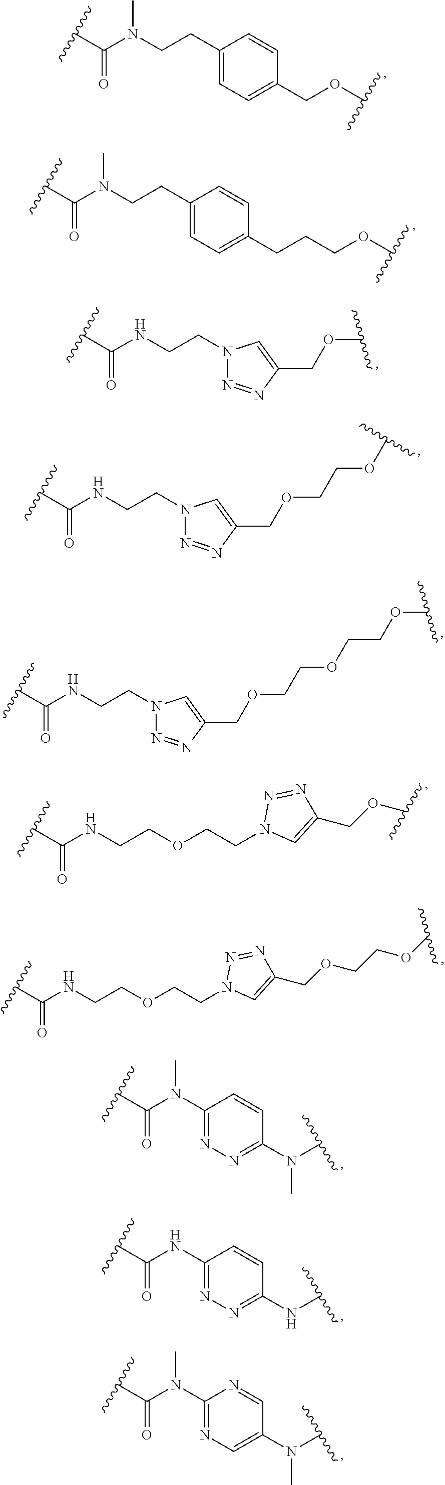

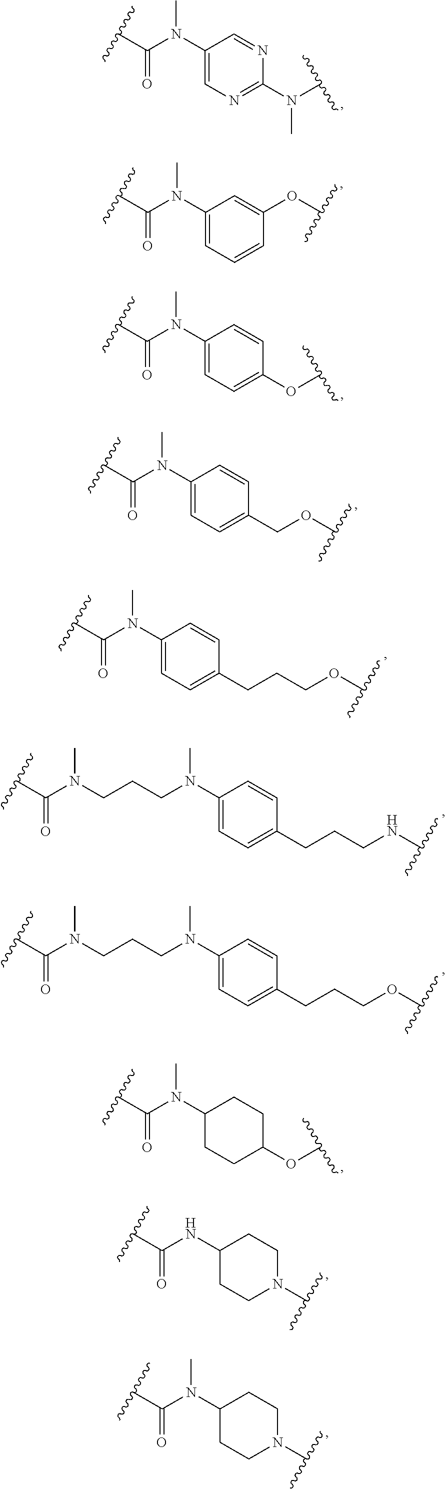

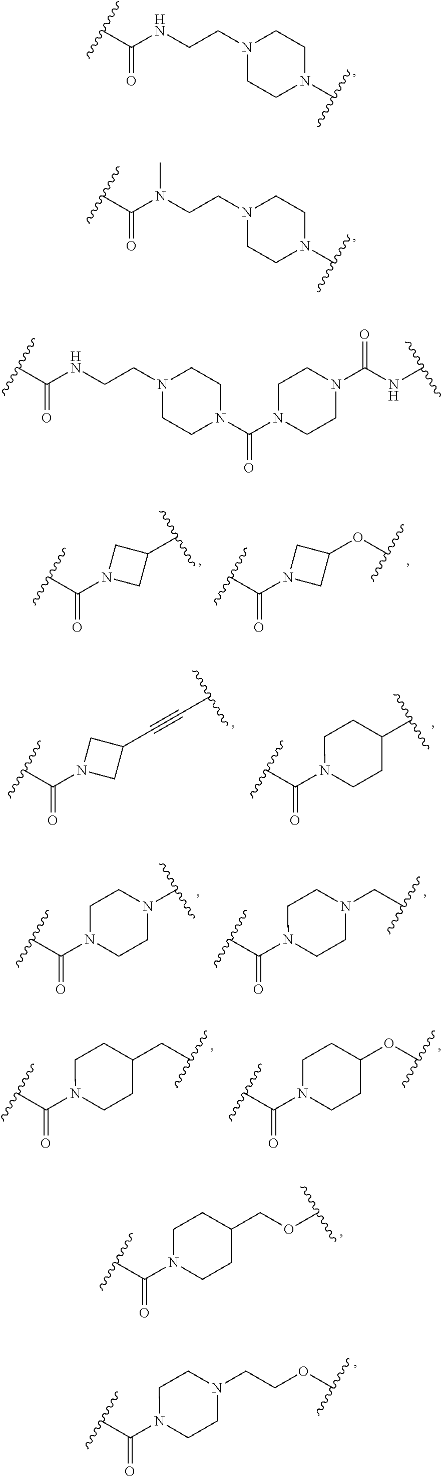

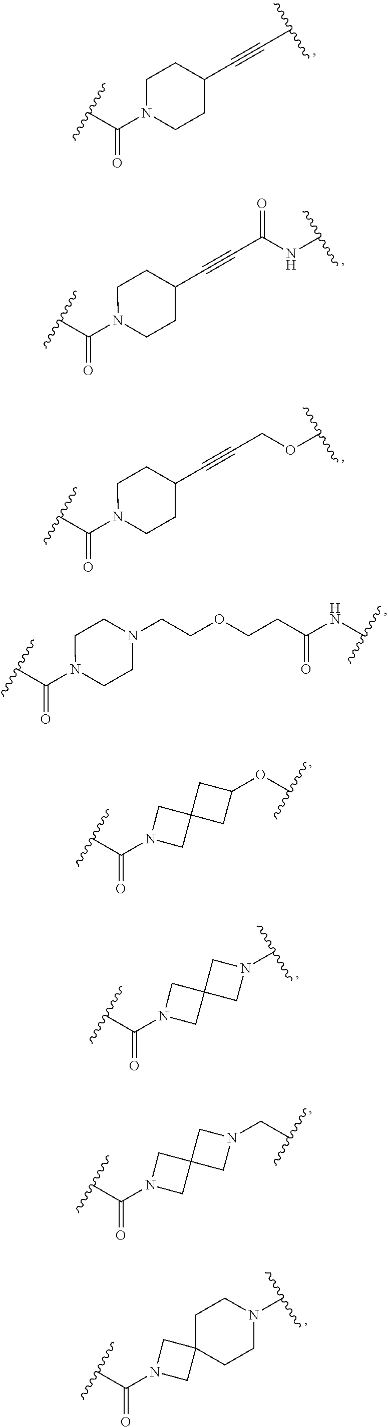

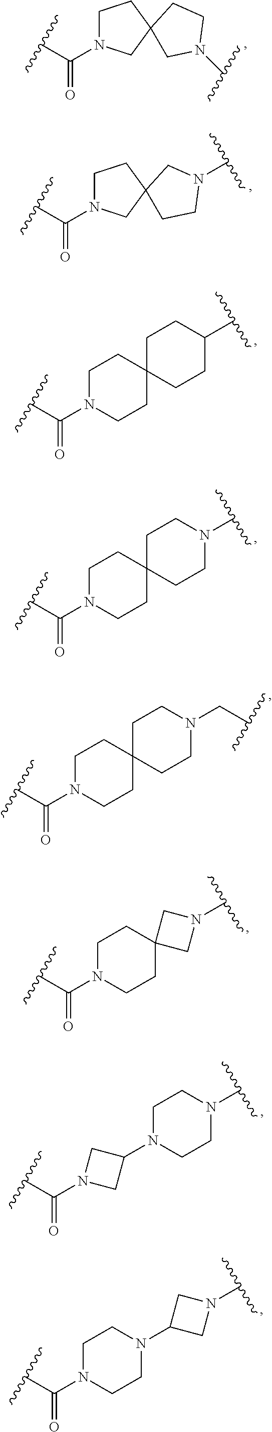

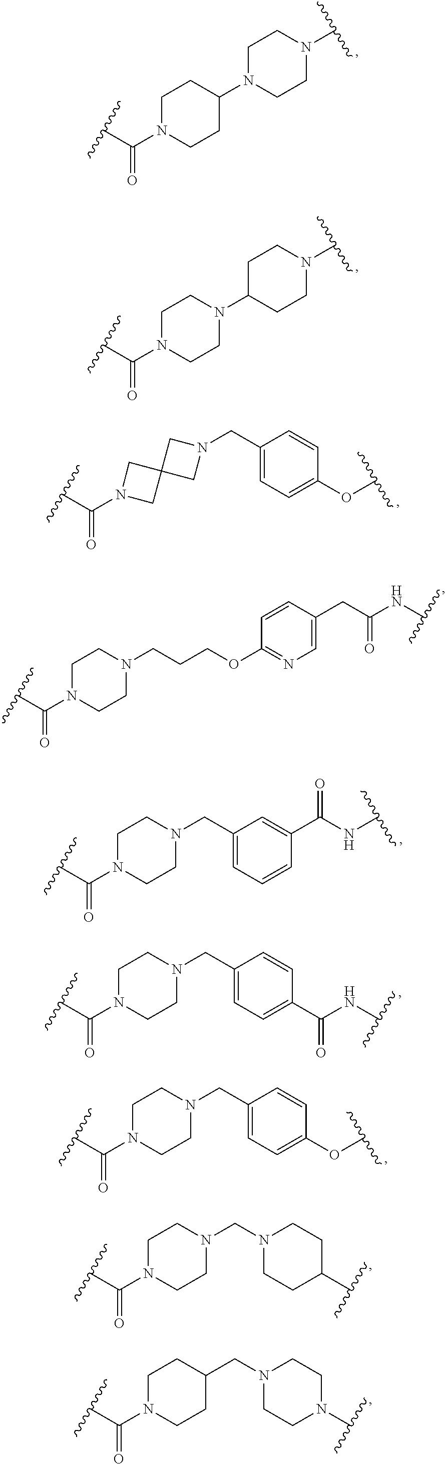

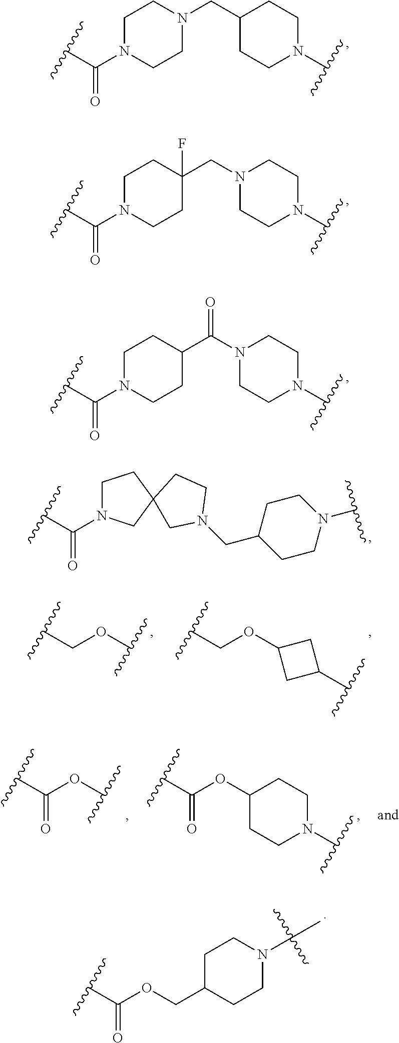

139. The compound of any one of Embodiments 1-65, wherein the linker is selected from: —CH₂—,

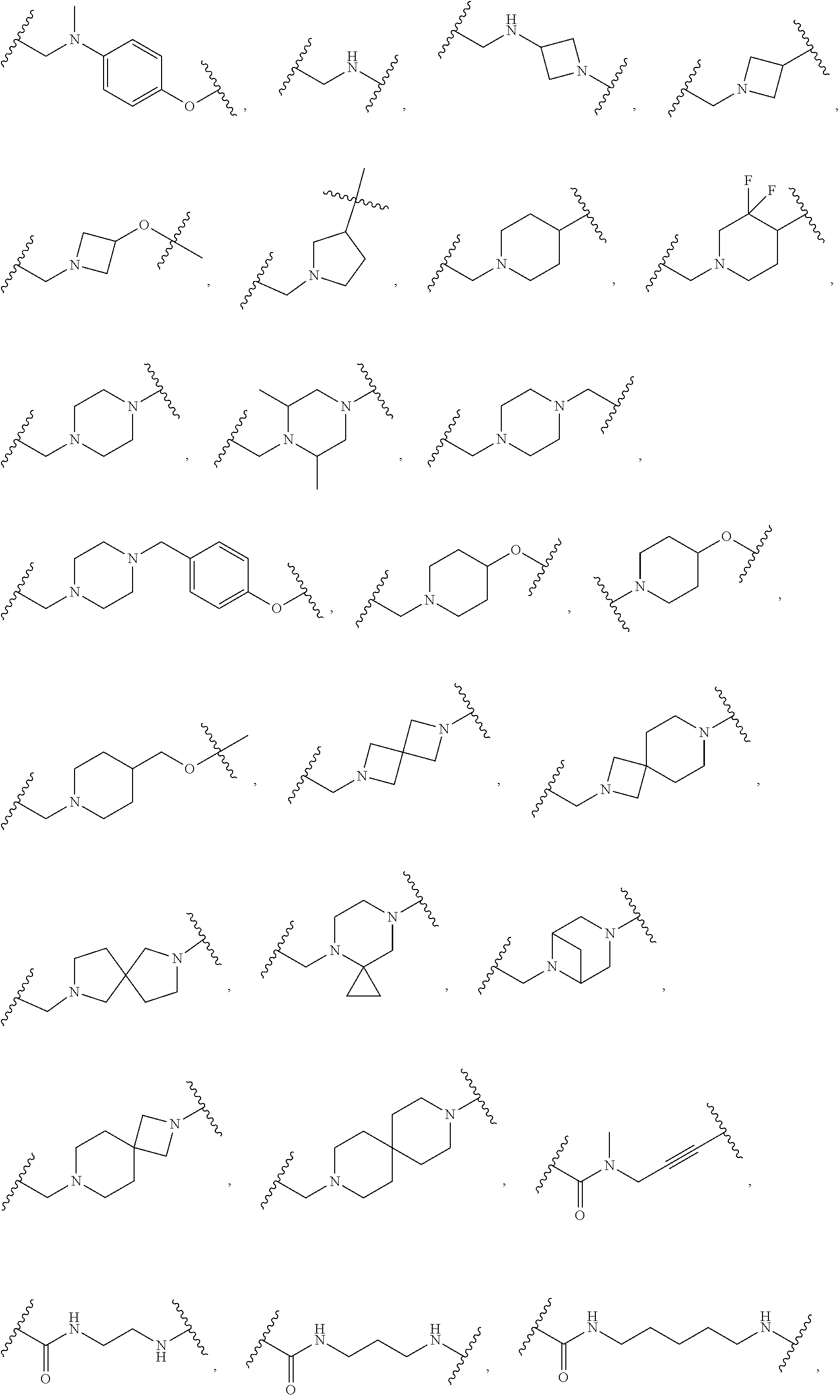

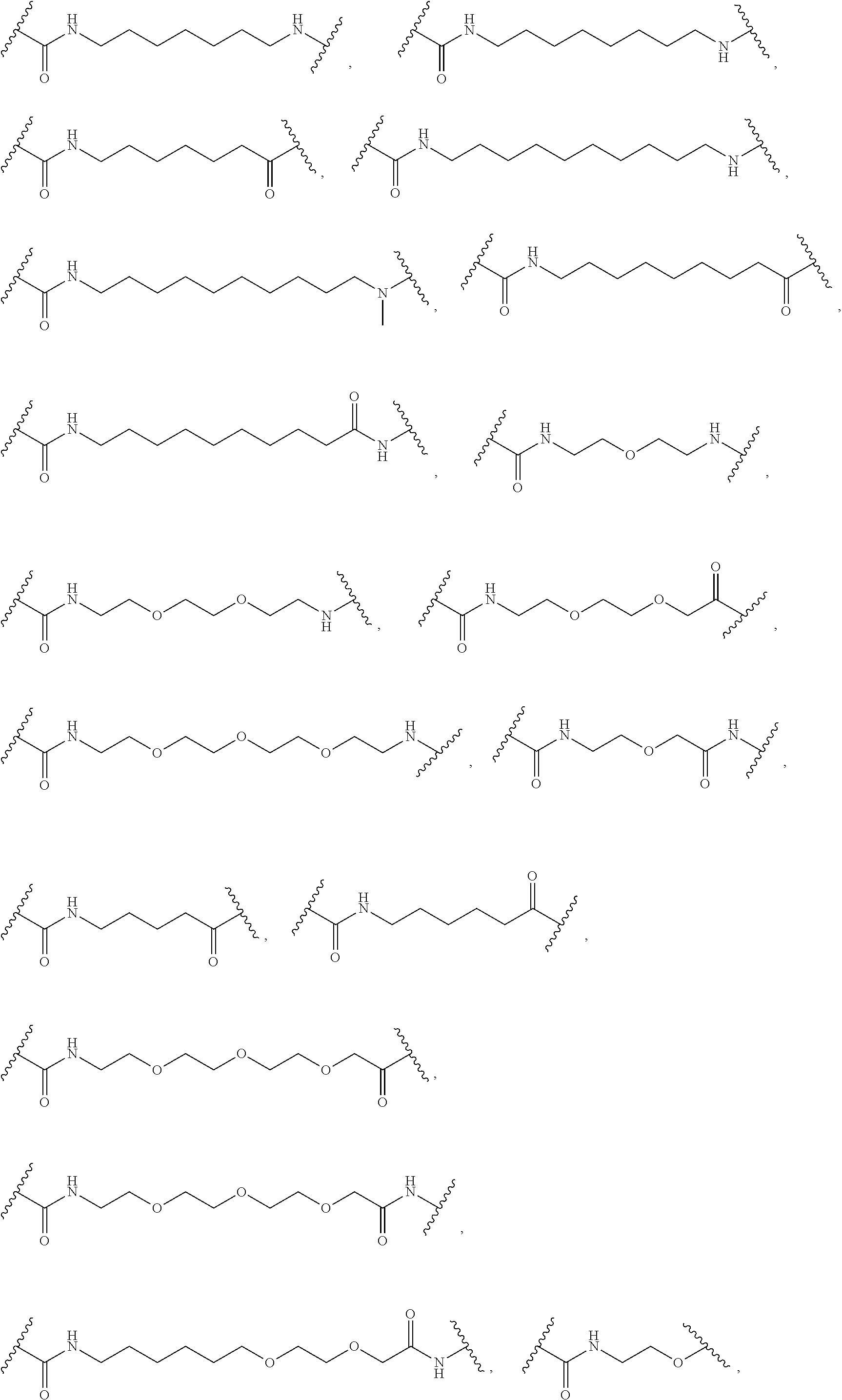

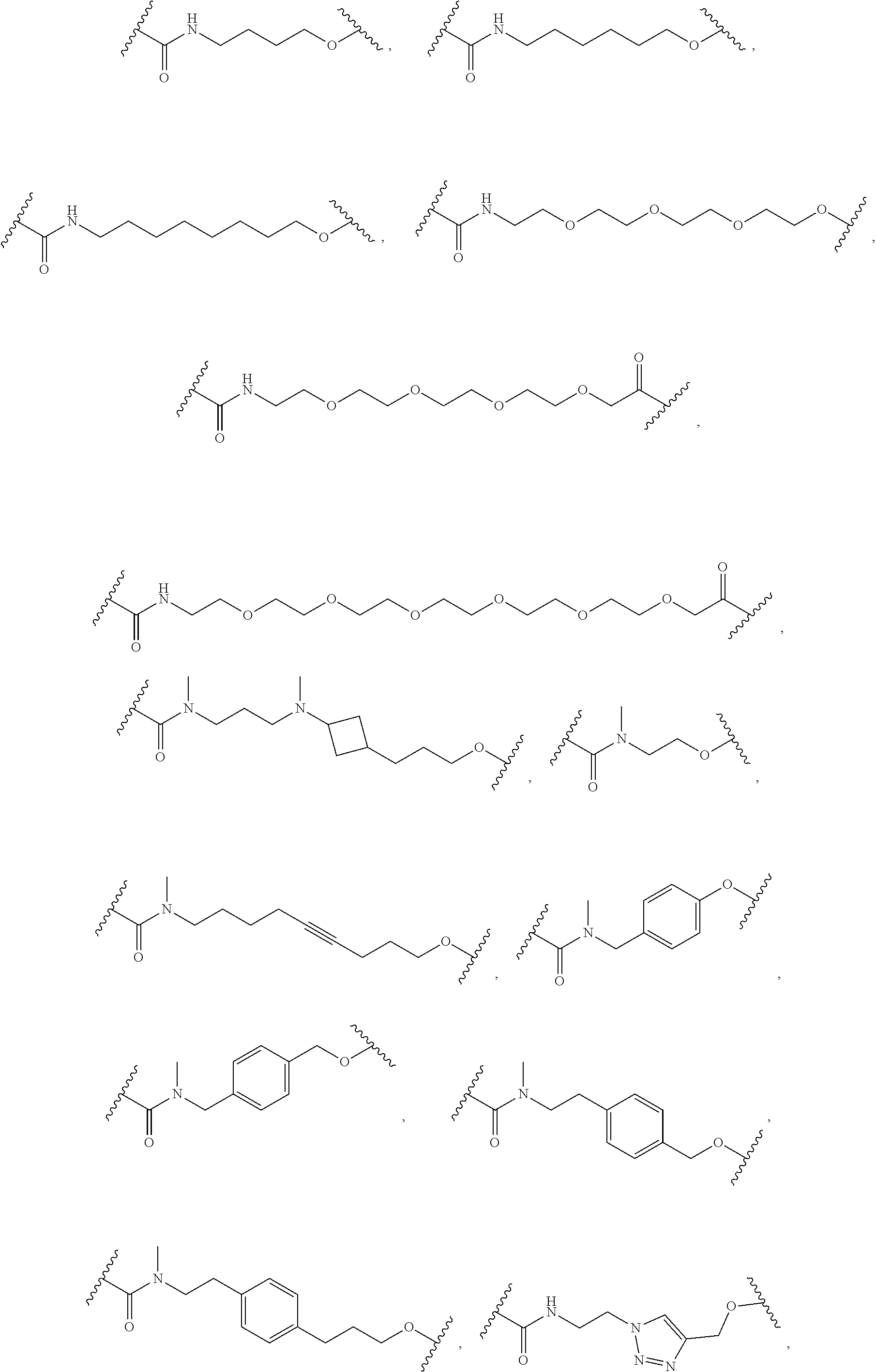

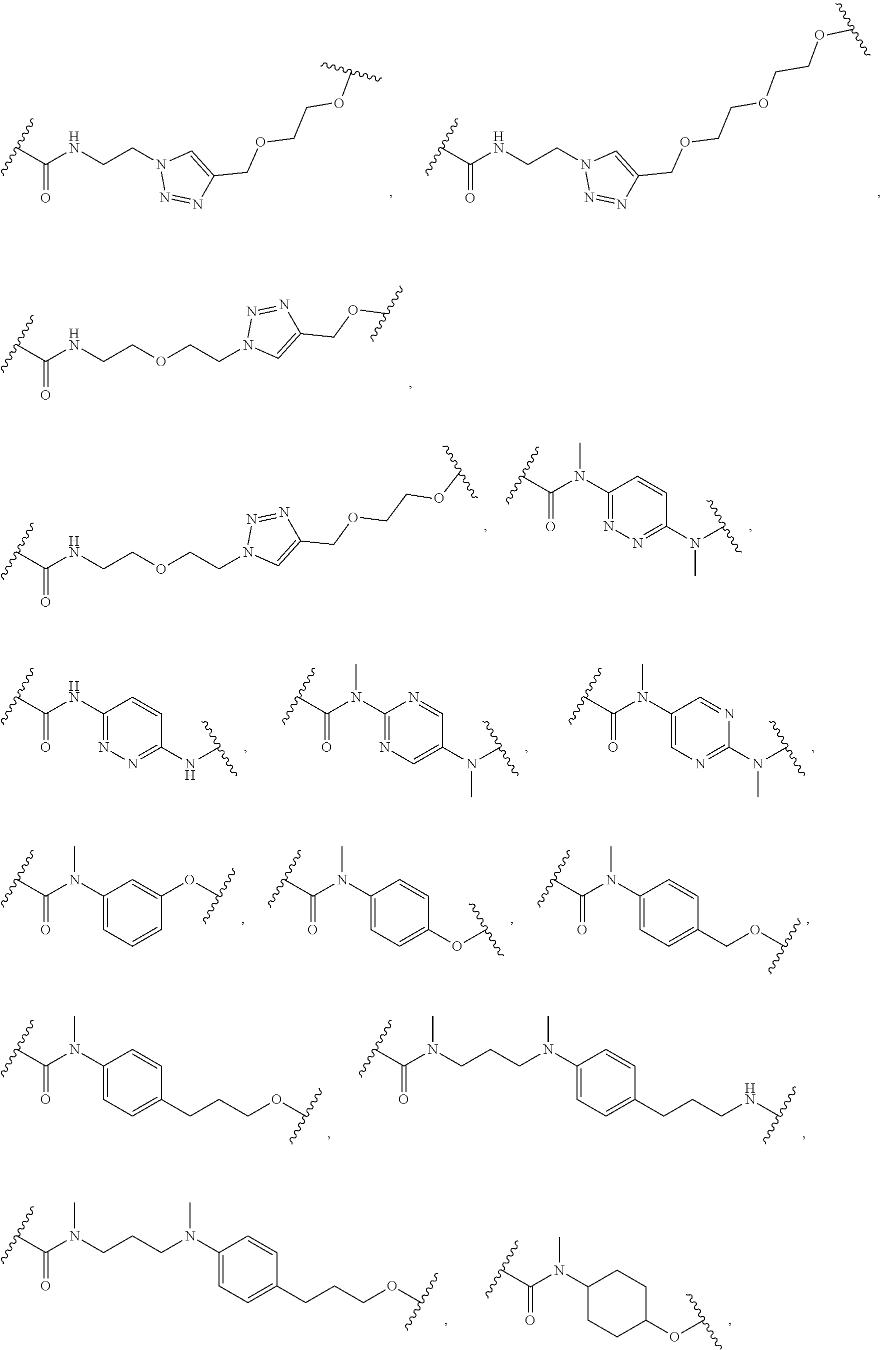

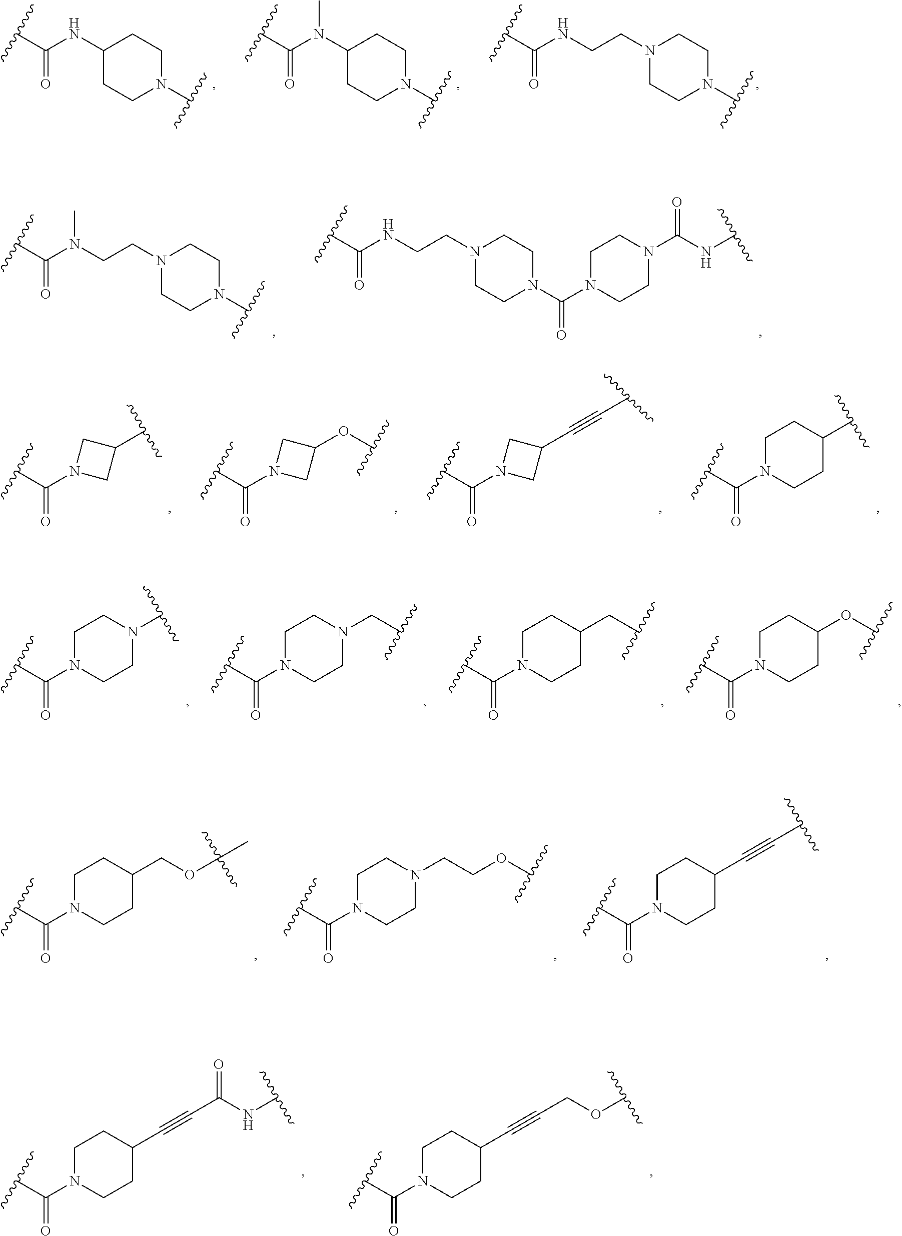

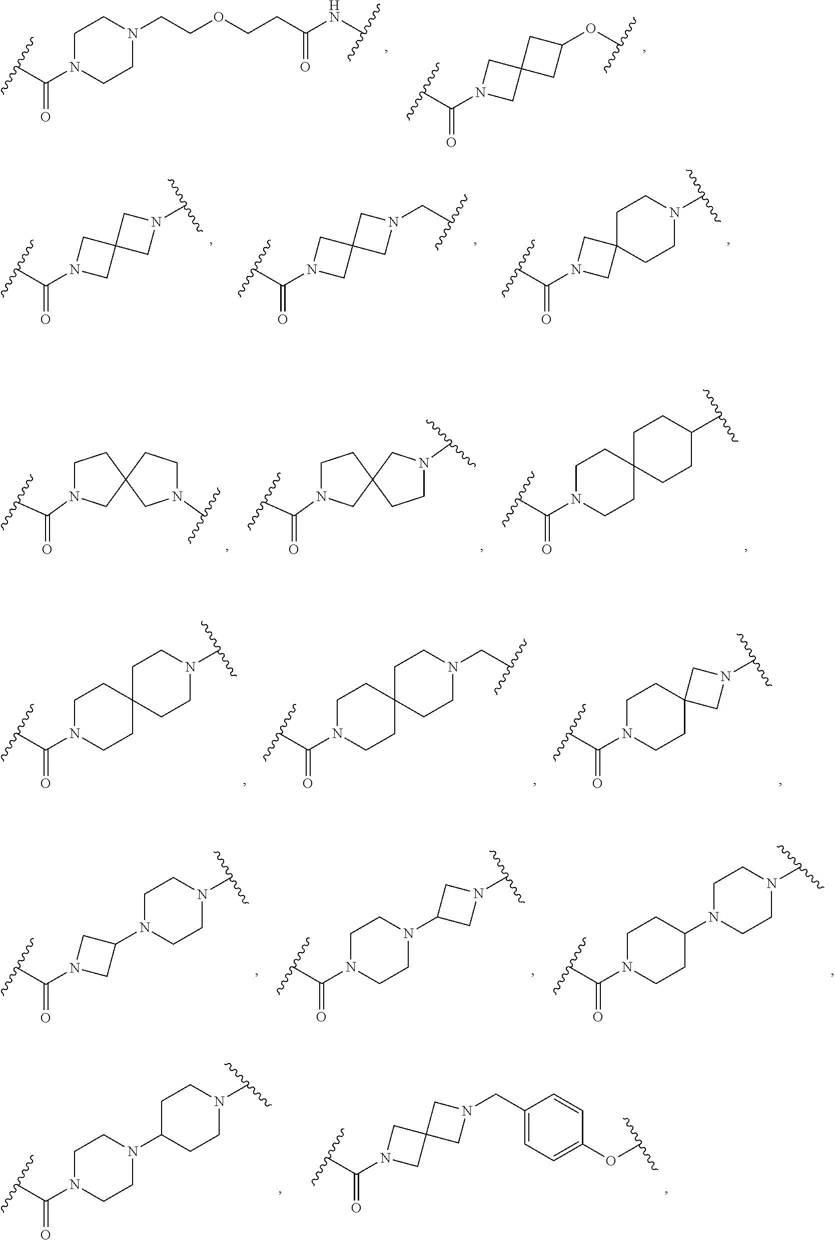

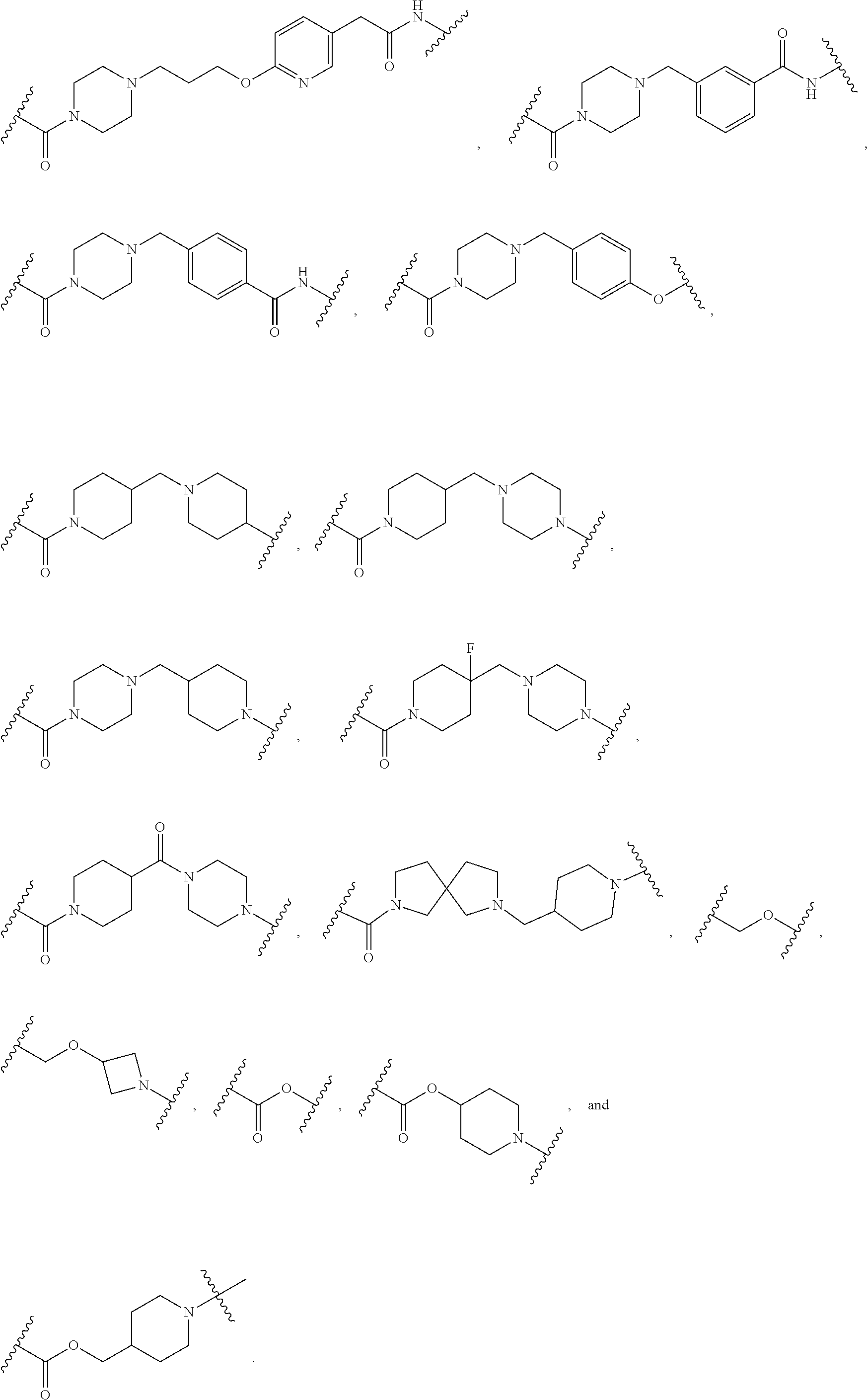

140. The compound of any one of Embodiments 1-65, wherein the linker is selected from

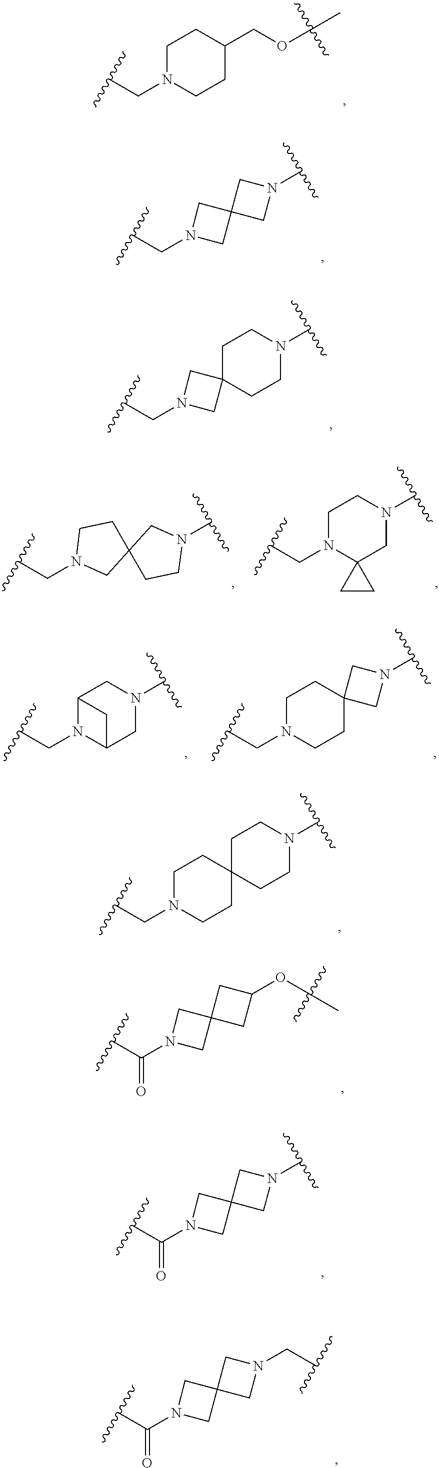

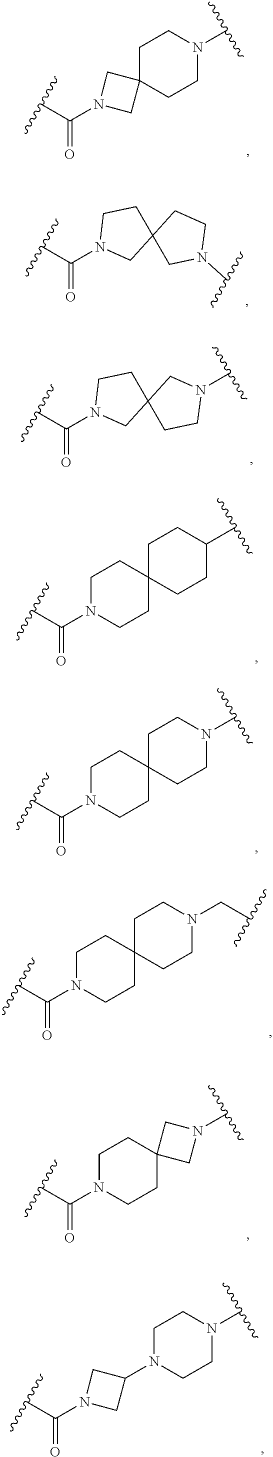

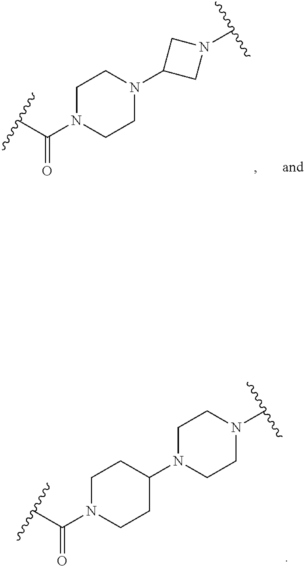 [SMILES: *C(=O)N1CCN(C2CN(*)C2)CC1 ; *C(=O)N1CCC(N2CCN(*)CC2)CC1]

141. The compound of any one of Embodiments 1-140, wherein LBM is an E3 ubiquitin ligase binding moiety.
142. The compound of any one of Embodiments 1-140, wherein LBM is a VHL binding moiety.
143. The compound of any one of Embodiments 1-140, wherein LBM is an IAP binding moiety.
144. The compound of any one of Embodiments 1-140, wherein LBM is a MDM2 binding moiety.
145. The compound of any one of Embodiments 1-140, wherein the compound is of Formula IIA:

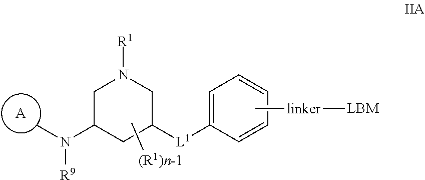

or a pharmaceutically acceptable salt thereof.
146. The compound of any one of Embodiments 1-140, wherein the compound is of Formula HA-1, IIA-2, IIA-3, or IIA-4:

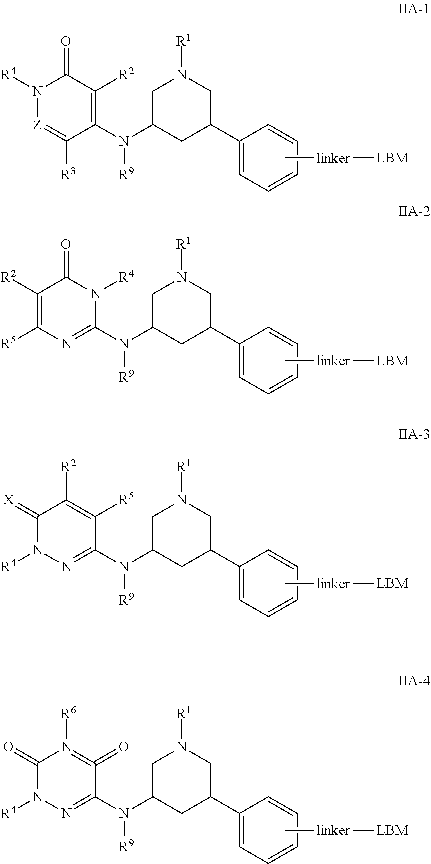

or a pharmaceutically acceptable salt thereof.
147. The compound of any one of Embodiments 1-140, wherein the compound is of Formula IIA-5:

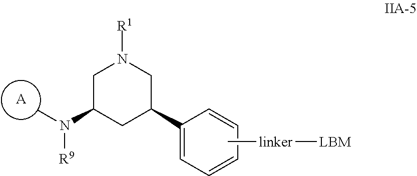

or a pharmaceutically acceptable salt thereof.
148. The compound of any one of Embodiments 1-140, wherein the compound is of Formula IIA-6:

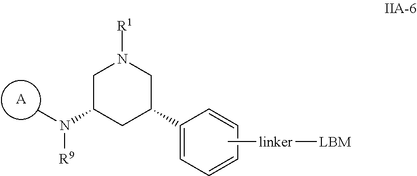

or a pharmaceutically acceptable salt thereof.
149. The compound of any one of Embodiments 1-140, wherein LBM is

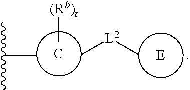

150. The compound of any one of Embodiments 1-140, wherein the compound is of Formula III-1:

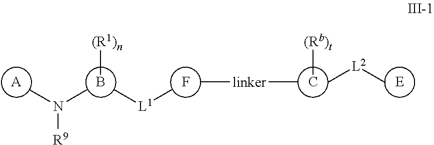

or a pharmaceutically acceptable salt thereof, wherein;
   each R^{b} is independently halogen, —CN, —R′ or —OR;
   t is 0, 1, 2, 3, or 4;
   L² is a covalent bond or an optionally substituted C₁₋₃ hydrocarbon chain, wherein one or more methylene units are optionally and independently replaced by —O—, —S—, —N(R)—, ~N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —S(O)—, —SO₂—, —SO₂N(R)—, —N(R)SO₂—, or -Cy-;
   Ring E is

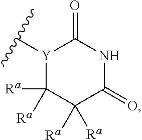

or an optionally substituted group selected from

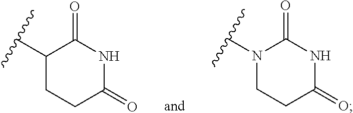 [SMILES: *C1CCC(=O)NC1=O ; "and" ; *N1CCC(=O)NC1=O]

Y is N or CH; and
each R^{a} is independently halogen, —CN or —R′.
151. The compound of Embodiment 150, wherein t is 1.
152. The compound of Embodiment 150, wherein t is 0.
153. The compound of any one of Embodiments 1-140, wherein the compound is of Formula IIIL:

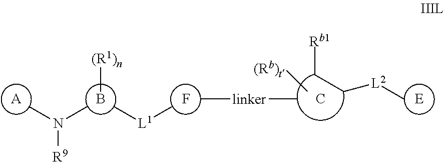

or a pharmaceutically acceptable salt thereof, wherein:
   each R^{b} and R^{b1} is independently halogen, —CN, —R′ or —OR;
   t′ is 0, 1, 2, or 3;
   L² is a covalent bond or an optionally substituted Cis hydrocarbon chain, wherein one or more methylene units are optionally and independently replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —S(O)—, —SO₂—, —SO₂N(R)—, —N(R)SO₂—, or -Cy-;
   Ring E is

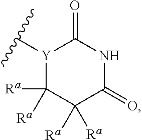

or an optionally substituted group selected from

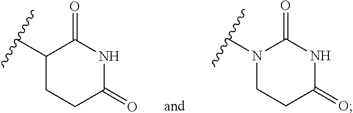 [SMILES: *C1CCC(=O)NC1=O ; "and" ; *N1CCC(=O)NC1=O]

Y is N or CH; and
each R^{a} is independently halogen, —CN or —R′.
154. The compound of Embodiment 153, wherein t′ is 0.
155. The compound of any one of Embodiments 153-154, wherein R^{b1} is not —H.
156. The compound of any one of Embodiments 153-154, wherein R^{b1} is optionally substituted C₁₋₆ aliphatic.
157. The compound of any one of Embodiments 153-154, wherein R^{b1} is optionally substituted C₁₋₆ alkyl.
158. The compound of any one of Embodiments 153-154, wherein R^{b1} is C₁₋₆ alkyl.
159. The compound of any one of Embodiments 153-154, wherein R^{b1} is C₁₋₆ haloalkyl.
160. The compound of any one of Embodiments 153-154, wherein R^{b1} is —OR wherein R is optionally substituted C₁₋₆ aliphatic.
161. The compound of any one of Embodiments 153-154, wherein R^{b1} is —OR wherein R is optionally substituted C₁₋₆ alkyl.
162. The compound of any one of Embodiments 153-154, wherein R^{b1} is —OR wherein R is C₁₋₆ alkyl.
163. The compound of any one of Embodiments 153-154, wherein R^{b1} is —OR wherein R is C₁₋₆ haloalkyl.
164. The compound of any one of Embodiments 153-154, wherein R^{b1} is halogen.
165. The compound of any one of Embodiments 153-154, wherein R^{b1} is —OCF₃.
166. The compound of any one of Embodiments 153-154, wherein R^{b1} is —OCH₃.
167. The compound of any one of Embodiments 153-154, wherein R^{b1} is —CF₃.
168 The compound of any one of Embodiments 153-154, wherein R^{b1} is —CH₃.
169. The compound of any one of Embodiments 153-154, wherein R^{b1} is —F.
170. The compound of any one of the preceding Embodiments, wherein Ring C is aromatic.
171. The compound of Embodiment 170, wherein Ring C is an optionally substituted phenyl ring.
172. The compound of Embodiment 170, wherein Ring C is phenyl ring.
173. The compound of Embodiment 170, wherein Ring C is optionally substituted

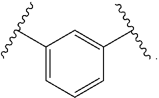 [SMILES: *c1cccc(*)c1]

174. The compound of Embodiment 170, wherein Ring C is optionally substituted

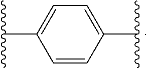 [SMILES: *c1ccc(*)cc1]

175. The compound of Embodiment 170, wherein Ring C is an optionally substituted 5-membered heteroaryl ring having 1-2 heteroatoms independently selected from N, O, and S.
176. The compound of Embodiment 170, wherein Ring C is an optionally substituted 6-membered heteroaryl ring having 1-2 heteroatoms independently selected from N, O, and S.
177. The compound of Embodiment 170, wherein Ring C is an optionally substituted 9-membered bicyclic heteroaryl ring having 1-4 heteroatoms independently selected from N, O, and S.
178. The compound of Embodiment 170, wherein Ring C is an optionally substituted 10-membered bicyclic heteroaryl ring having 1-4 heteroatoms independently selected from N, O, and S.
179. The compound of any one of Embodiments 2-178, wherein Ring E is

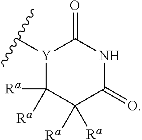

180. The compound of any one of Embodiments 2-178, wherein Ring E is an optionally substituted

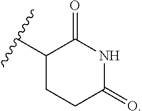 [SMILES: *C1CCC(=O)NC1=O]

181. The compound of any one of Embodiments 2-178, wherein Ring E is an optionally substituted

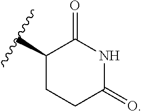 [SMILES: *[C@@H]1CCC(=O)NC1=O]

182. The compound of any one of Embodiments 2-178, wherein Ring E is an optionally substituted

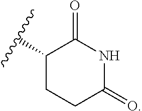 [SMILES: *[C@H]1CCC(=O)NC1=O]

183. The compound of any one of Embodiments 2-178, wherein Ring E is optionally substituted

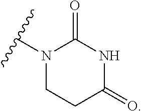 [SMILES: *N1CCC(=O)NC1=O]

184. The compound of any one of Embodiments 2-178, wherein Ring E is

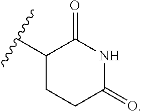 [SMILES: *C1CCC(=O)NC1=O]

185. The compound of any one of Embodiments 2-178, wherein Ring E is

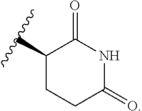 [SMILES: *[C@@H]1CCC(=O)NC1=O]

186. The compound of any one of Embodiments 2-178, wherein Ring E is

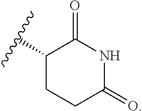 [SMILES: *[C@H]1CCC(=O)NC1=O]

187. The compound of any one of Embodiments 2-178, wherein Ring E is

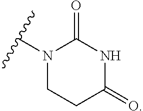 [SMILES: *N1CCC(=O)NC1=O]

188. The compound of any one of Embodiments 1-140, wherein the compound is of Formula III:

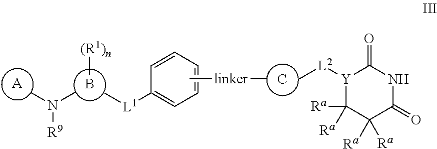

or a pharmaceutically acceptable salt thereof, wherein:
   Ring C is an optionally substituted, mono- or multicyclic, 3- to 16-membered bivalent ring system, wherein the ring system is fully saturated, partially saturated, or aromatic, and the ring system contains 0-6 heteroatoms independently selected from N, O, and S;
   each R^{a} is independently hydrogen or an optionally substituted C₁₋₆ aliphatic, or two R^{a} groups, together with the atom(s) to which they are attached, combine to form a 3- to 6-membered saturated or partially unsaturated ring;
   L² is a covalent bond or a straight or branched C₁₋₃ hydrocarbon chain wherein one methylene is optionally replaced with —O—, —S—, —N(R)—, —SO₂—, —C(O)N(R)—, or —N(R)C(O)—; and
   Y is N or CH.
189. The compound of Embodiment 188, wherein Ring C is optionally substituted phenyl, C₅₋₆ cycloaliphatic, 5- to 6-membered heteroaryl having 1-4 heteroatoms independently selected from N, O, and S, 5- to 6-membered heterocyclyl having 1-2 heteroatoms independently selected from N, O, and S, or 9- to 10-membered bicyclic heteroaryl having 1-4 heteroatoms independently selected from N, O, and S.
190. The compound of Embodiment 188, wherein Ring C is optionally substituted phenyl or 5- to 6-membered heteroaryl having 1-4 heteroatoms independently selected from N, O, and S.
191. The compound of Embodiment 188, wherein Ring C is

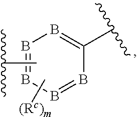

wherein:
   each B is independently selected from N, C, and CH, provided that no more than two B are N;
   each R^{c} is independently selected from halogen, —OR, —N(R)₂, —CN, and optionally substituted C₁₋₆ aliphatic; and
   m is 0, 1, 2, or 3,
192. The compound of Embodiment 188, wherein Ring C is

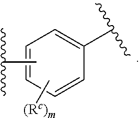

193. The compound of Embodiment 188, wherein the compound is of Formula IIIC:

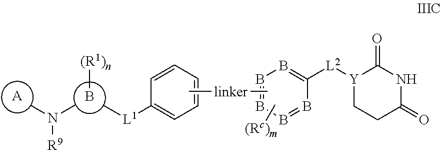

or a pharmaceutically acceptable salt thereof.
194. The compound of Embodiment 188, wherein the compound is of Formula IIID:

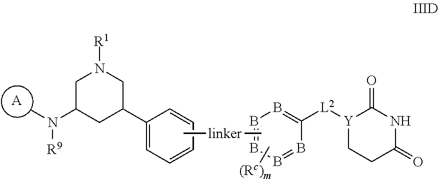

or a pharmaceutically acceptable salt thereof.
195. The compound of Embodiment 188, wherein Ring C is optionally substituted C₃-C₇ cycloaliphatic or 3- to 7-membered heterocyclyl having 1-2 heteroatoms independently selected from N, O, and S.
196 The compound of Embodiment 188, wherein Ring C is optionally substituted 9- to 10-membered bicyclic heteroaryl having 1-4 heteroatoms independently selected from N, O, and S.
197. The compound of Embodiment 188, wherein Ring C is:

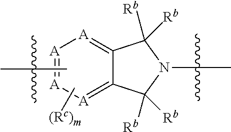

wherein:
   each A is independently N, C, or CH, provided that no more than two A groups are N;
   each R^{b} is hydrogen, or two R^{b} groups, on the same carbon, are taken together to form an oxo or combine to form a 3- to 6-membered saturated or partially unsaturated ring;
   each R^{c} is independently selected from halogen, —OR, —N(R)₂, —CN, and optionally substituted C₁₋₆ aliphatic; and
   m is 0, 1, 2, or 3.
198. The compound of Embodiment 197, wherein Ring C is:

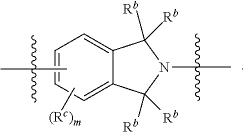

199. The compound of any one of Embodiments 2-198, wherein L² is a covalent bond.
200. The compound of any one of Embodiments 2-198, wherein L² is optionally substituted —CH₂—.
201. The compound of any one of Embodiments 2-198, wherein L² is —CH₂—.
202. The compound of any one of Embodiments 2-198, wherein L² is —O—.
203. The compound of any one of Embodiments 2-198, wherein L² is —S—.
204. The compound of any one of Embodiments 2-198, wherein L² is —C(O)N(R)—, or —N(R)C(O)—.
205. The compound of any one of Embodiments 2-198, wherein L² is —C(O)NH—, or —NHC(O)—.
206. The compound of any one of Embodiments 2-198, wherein L² is —N(R)—.
207. The compound of any one of Embodiments 2-198, wherein L² is —NH—.
208. The compound of any one of Embodiments 2-198, wherein L² is -Cy-.
209. The compound of any one of Embodiments 2-198, wherein L² is -Cy-, wherein -Cy- is aromatic.
210. The compound of any one of Embodiments 188-209, wherein Y is N.
211. The compound of any one of Embodiments 188-209, wherein Y is CH.
212. The compound of any one of Embodiments 188-211, wherein each R^{c} is independently selected from halogen, —O(C₁₋₆ alkyl), C₁₋₆ alkyl, and C₁₋₆ haloalkyl.
213. The compound of any one of Embodiments 196-212, wherein the compound is of Formula IIIA:

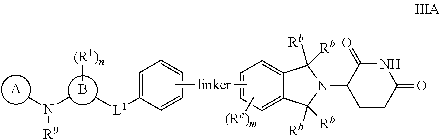

or a pharmaceutically acceptable salt thereof.
214. The compound of Embodiment 213, wherein the compound is of Formula IIIB:

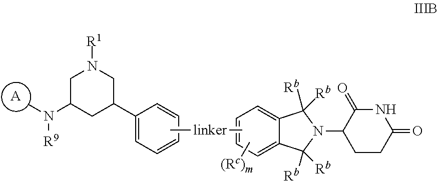

or a pharmaceutically acceptable salt thereof.
215. The compound of Embodiment 214, wherein the compound is of Formula IIIB-1 or IIIB-2:

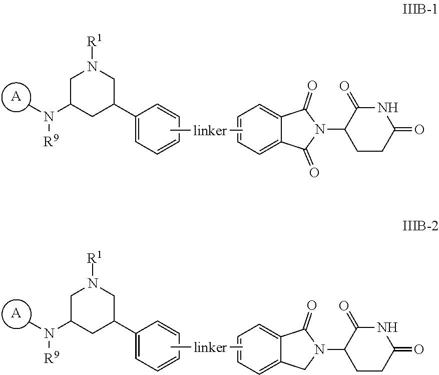

or a pharmaceutically acceptable salt thereof.
216. The compound of any one of the preceding Embodiments, wherein LBM or

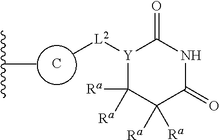

moiety is selected from

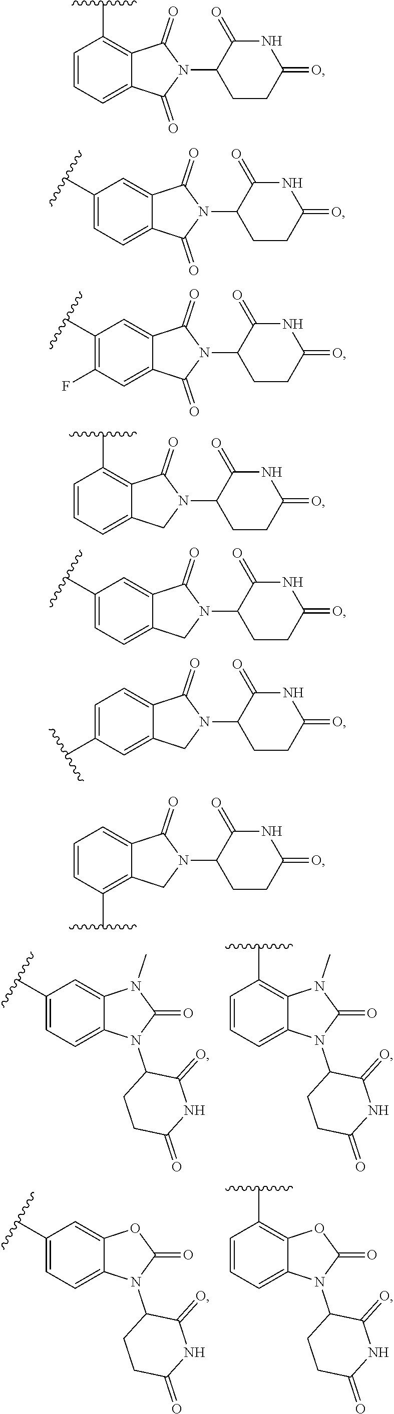

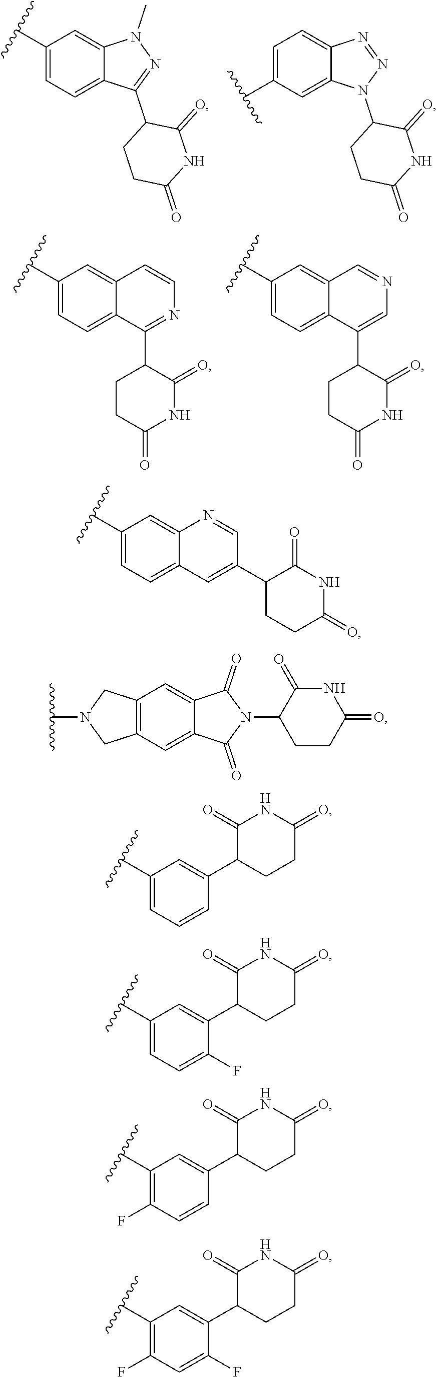

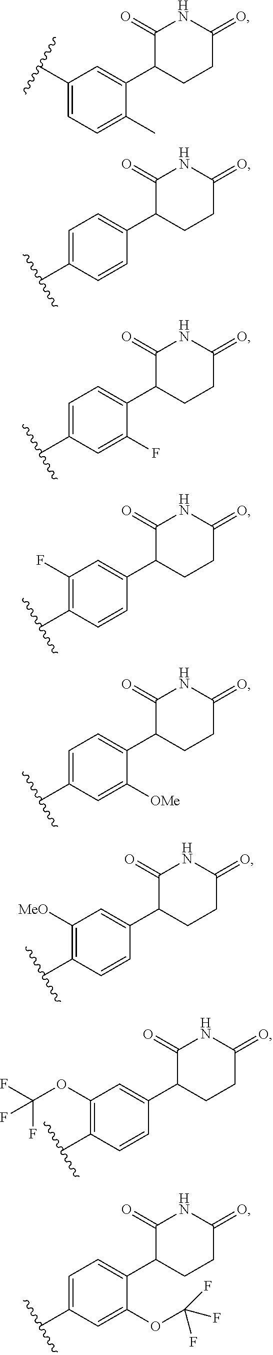

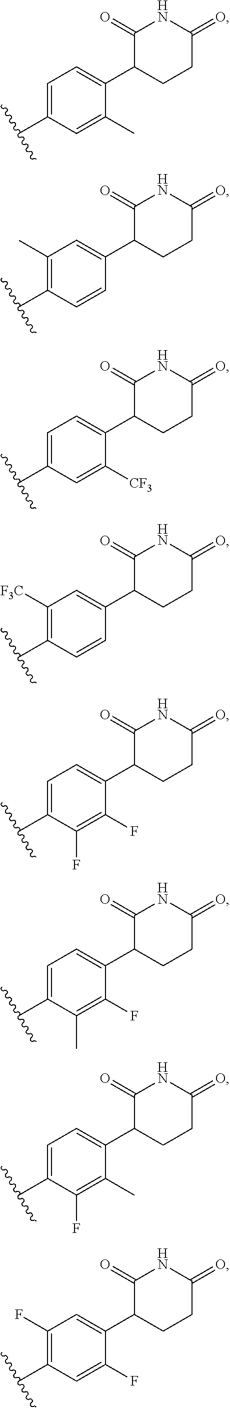

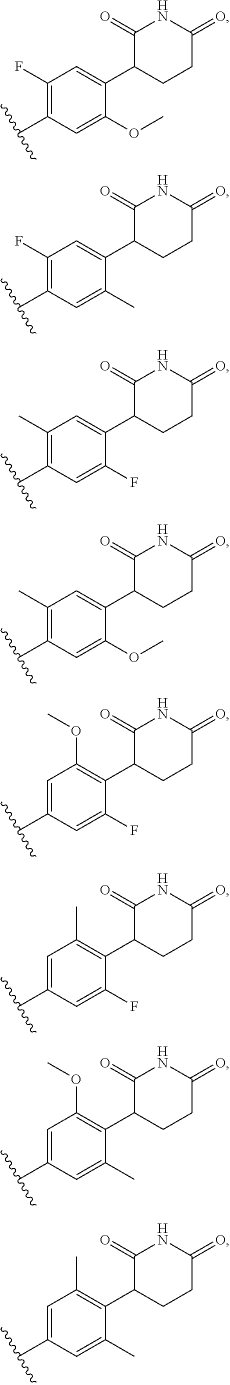

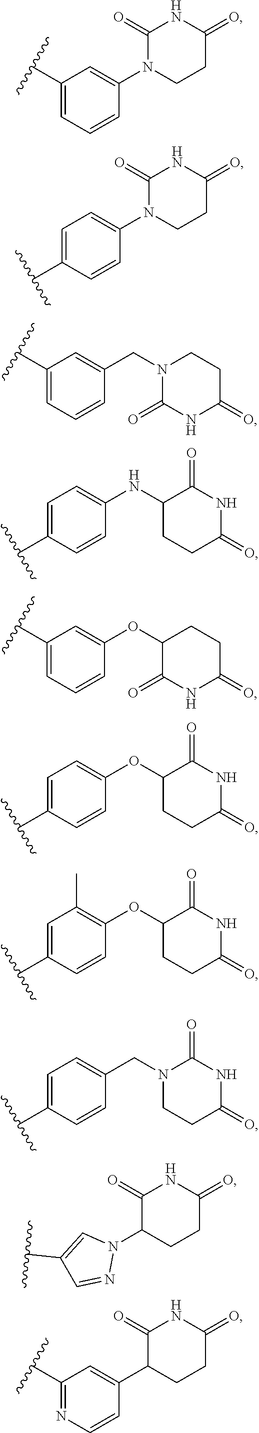

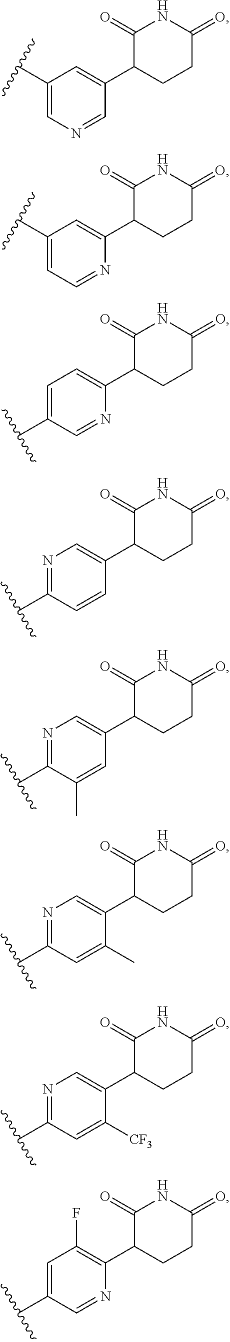

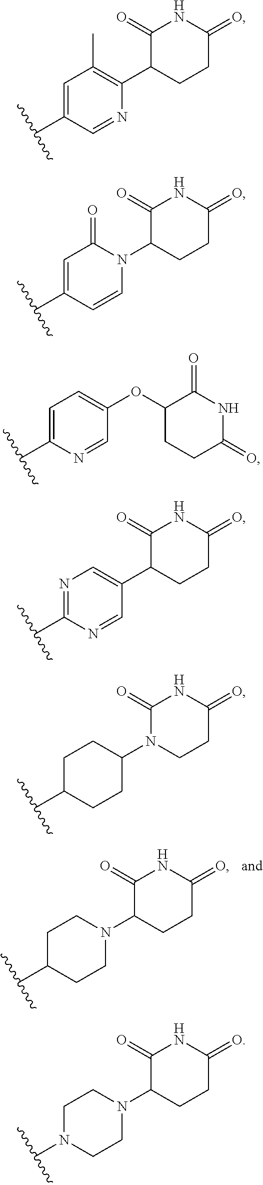

217. The compound of any one of Embodiments 1-215, wherein LBM or

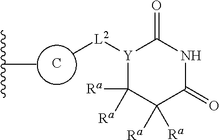

moiety is selected from:

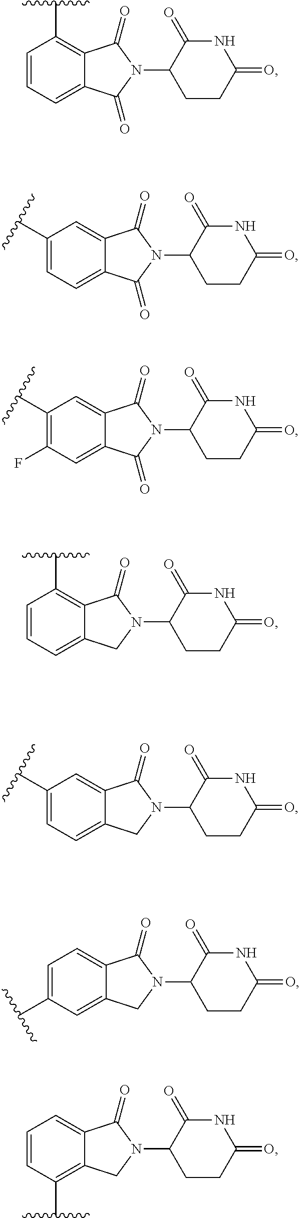

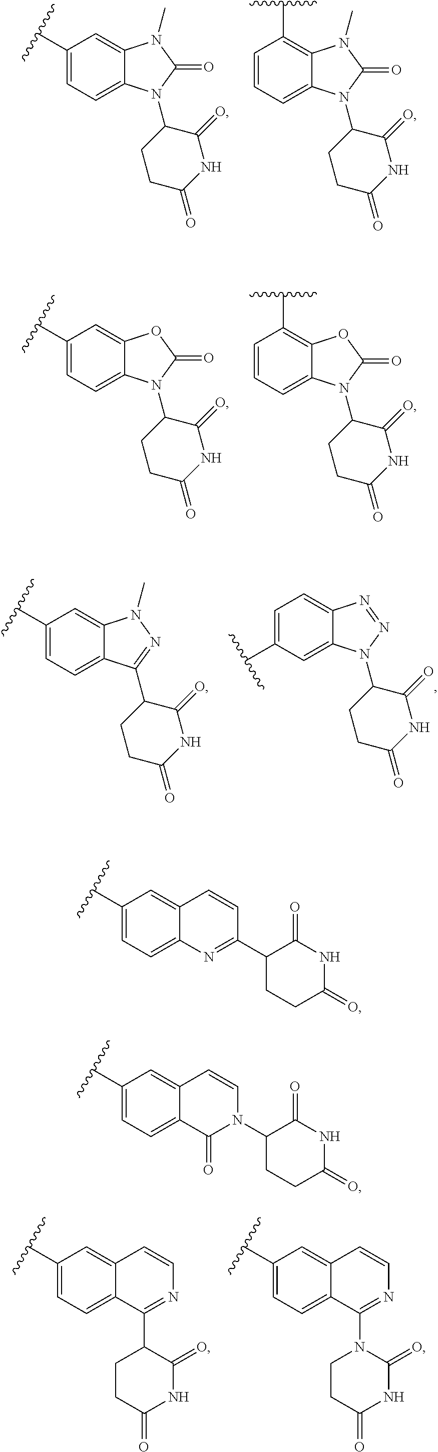

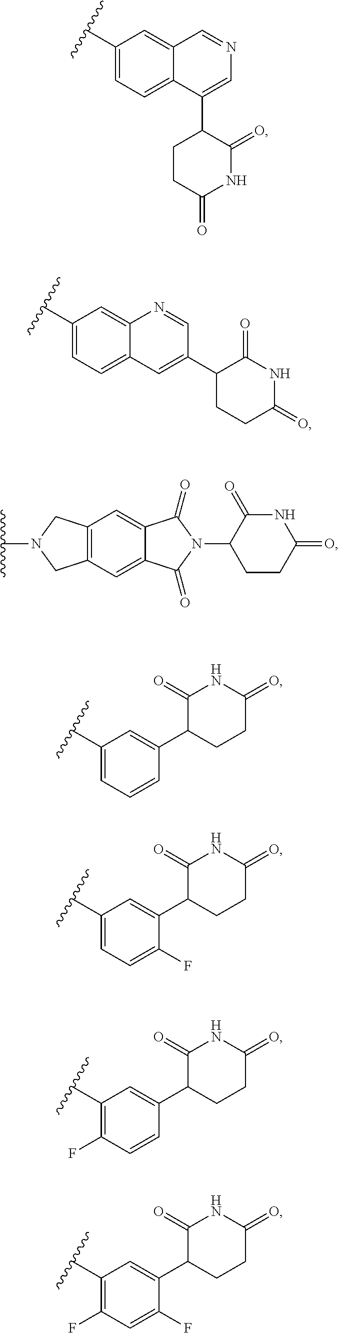

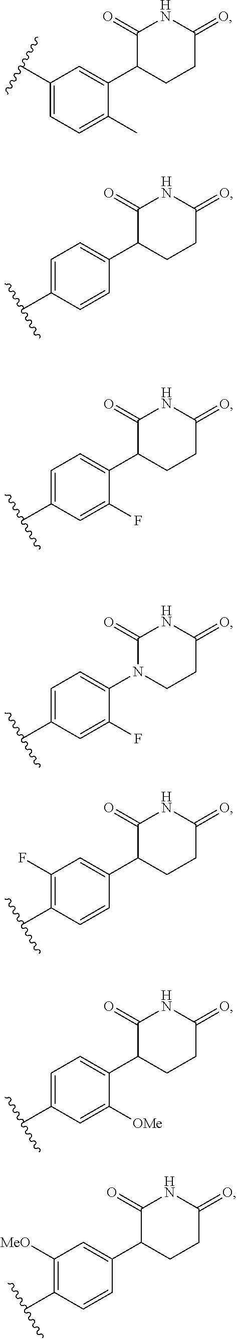

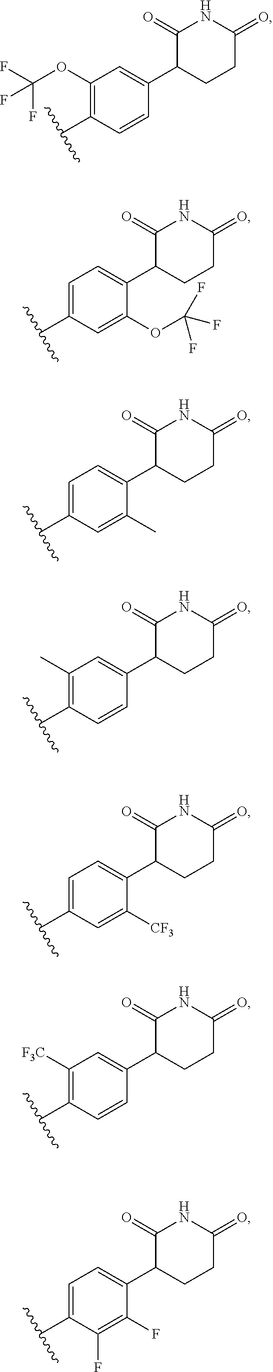

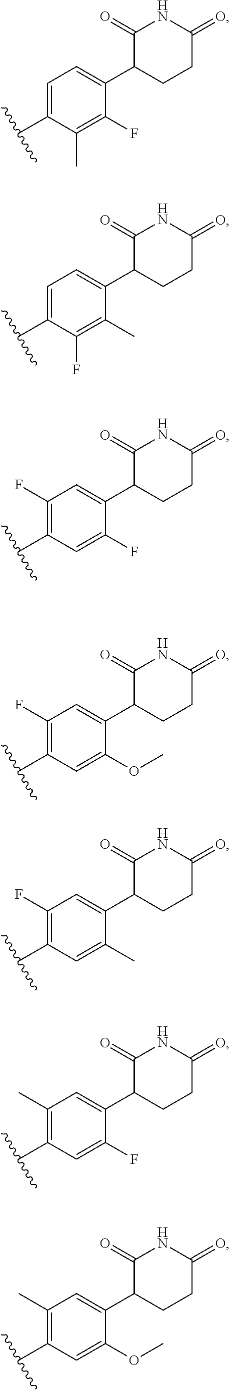

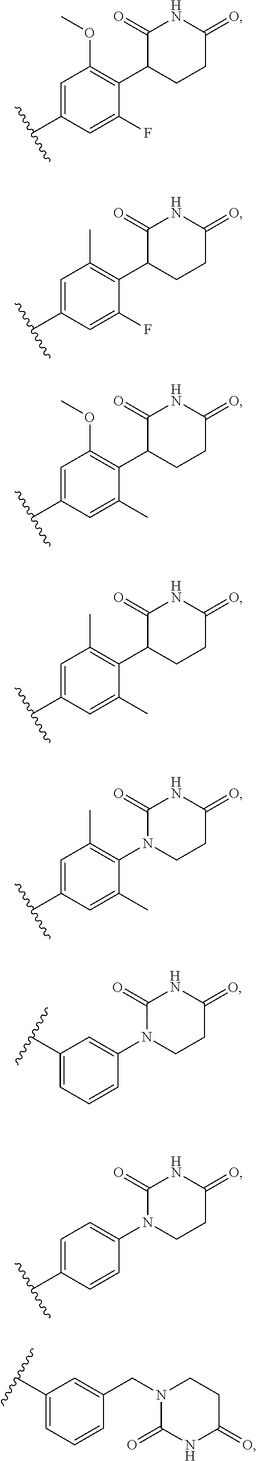

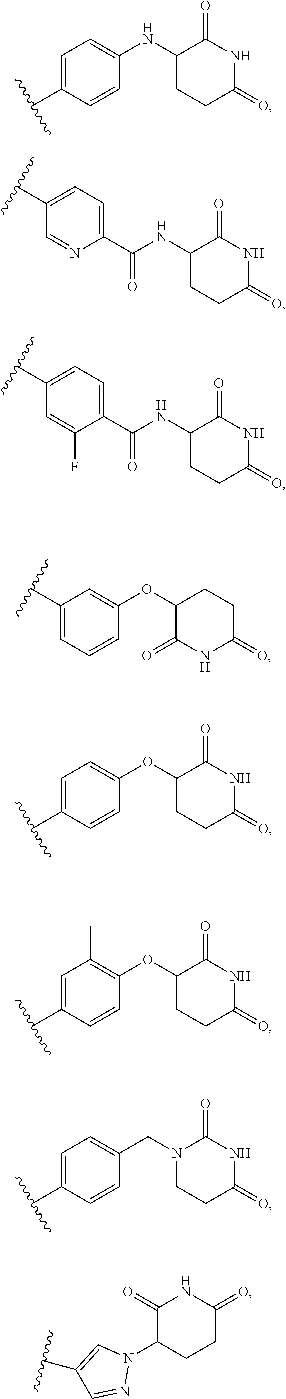

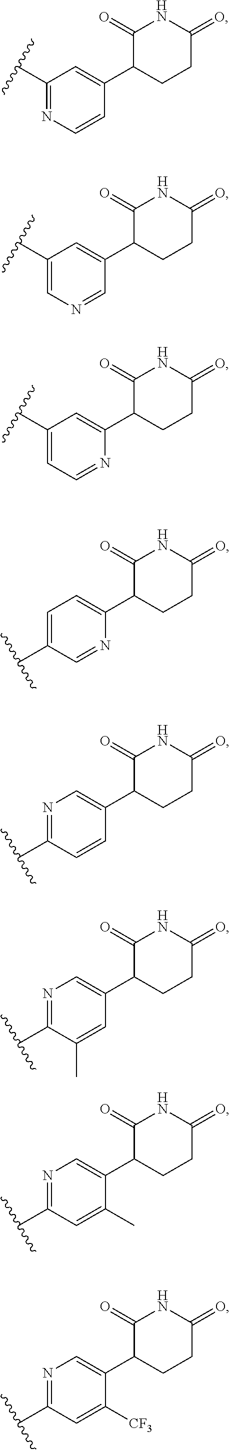

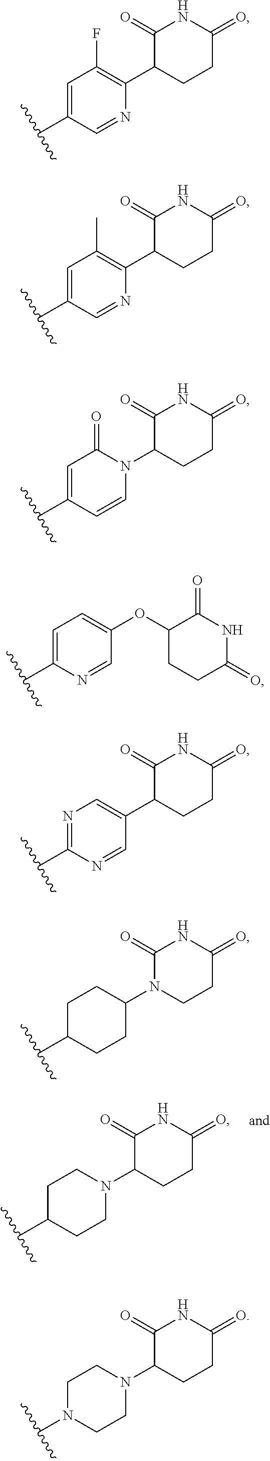

218. The compound of any one of the preceding Embodiments, wherein the compound is not:

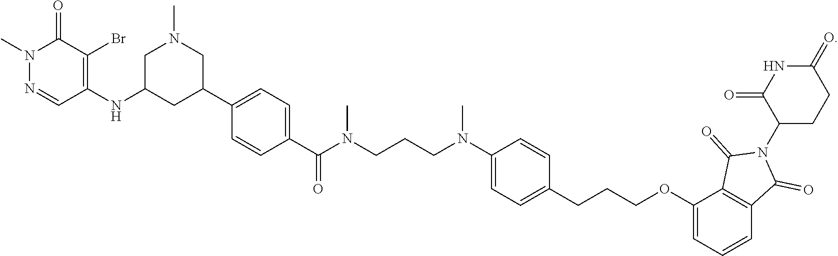 [SMILES: CN1CC(Nc2cnn(C)c(=O)c2Br)CC(c2ccc(C(=O)N(C)CCCN(C)c3ccc(CCCOc4cccc5c4C(=O)N(C4CCC(=O)NC4=O)C5=O)cc3)cc2)C1]

219. The compound of any one of Embodiments 1-148, wherein LBM is

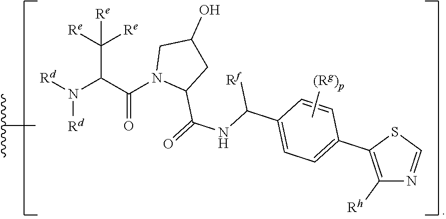

220. The compound of any one of Embodiments 1-148, wherein compound is of formula IV-1:

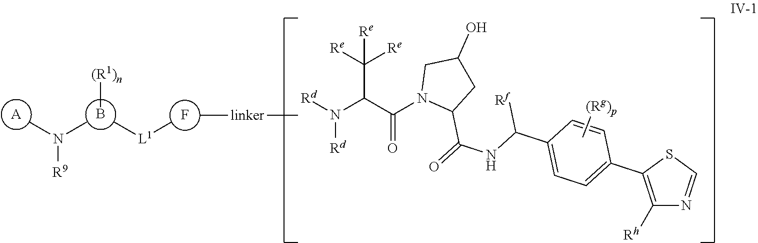

or a pharmaceutically acceptable salt thereof, wherein:
   linker is attached to the bracketed moiety at one of R^{d}, R^{e}, R^{f}, or R^{g};
   each R^{d} that is not the point of attachment for the linker is independently —R′, or
      two R^{d}, together with the atom to which they are attached, combine to form an optionally substituted 5- to 6-membered ring having 1-3 heteroatoms independently selected from N, O, and S and optionally fused to a phenyl or 5- to 6-membered heteroaryl ring having 1-3 heteroatoms independently selected from N, O, and S;
   each R^{e} that is not the point of attachment for the linker is independently —R;
   R^{f}, when it is not the point of attachment for the linker, is —R;
   each R^{g} that is not the point of attachment for the linker is independently halogen, —CN, R or —OR;
   p is 0, 1, 2, or 3;
   R^{h} is halogen, or —R;
   each R′ is independently —R, —C(O)R, or —S(O)₂R, or two R′ attached to the same atom, together with the atom to which they are attached, combine to form an optionally substituted 3- to 16-membered ring having 1-5 heteroatoms independently selected from N, O, and S; and
   each R is independently hydrogen or an optionally substituted group selected from C₁₋₈ aliphatic, C₃₋₁₀ cycloaliphatic, C₁-C₈ heteroaliphatic having 1-3 heteroatoms independently selected from N, O, and S, C₆₋₁₀ aryl, 5- to 10-membered heteroaryl having 1-5 heteroatoms independently selected from N, O, and S, and 3- to 10-membered monocyclic heterocyclyl having 1-5 heteroatoms independently selected from N, O, and S.
221. The compound of any one of Embodiments 1-148, wherein the compound is of Formula IV:

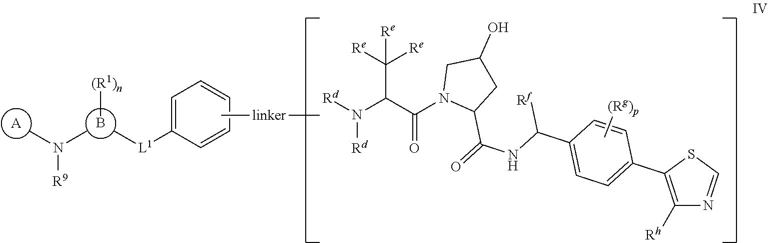

or a pharmaceutically acceptable salt thereof, wherein:
   linker is attached to the bracketed moiety at one of R^{d}, R^{e}, R^{f}, and R^{g};
   each R^{d} that is not the point of attachment for the linker is independently hydrogen, —C(O)R, or optionally substituted C₁₋₆ aliphatic, or
   two R^{d}, together with the atom to which they are attached, combine to form an optionally substituted 5- to 6-membered ring having 1-3 heteroatoms independently selected from N, O, and S and optionally fused to a phenyl or 5- to 6-membered heteroaryl ring having 1-3 heteroatoms independently selected from N, O, and S;
   each R^{e} that is not the point of attachment for the linker is independently hydrogen or optionally substituted C₁₋₆ aliphatic;
   R^{f}, when it is not the point of attachment for the linker, is hydrogen or optionally substituted C₁₋₆ aliphatic;
   each R^{g} that is not the point of attachment for the linker is independently halogen, —OR, —CN, or optionally substituted C₁₋₆ aliphatic;
   R^{h} is hydrogen, halogen, or optionally substituted C₁₋₆ aliphatic; and
   p is 0, 1, 2, or 3.
222. The compound of Embodiment 221, wherein the compound is of Formula IVA:

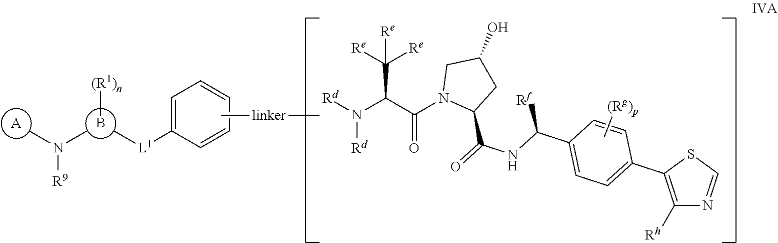

or a pharmaceutically acceptable salt thereof.
223. The compound of Embodiment 221, wherein the compound is of Formula IVA-1:

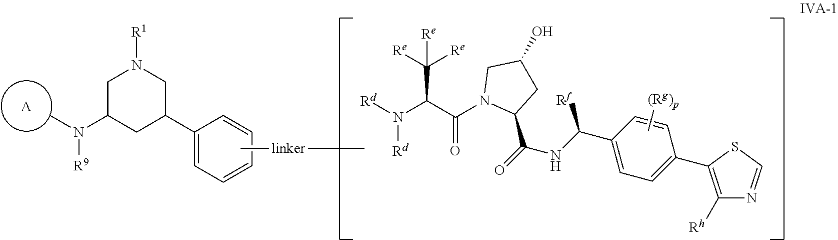

or a pharmaceutically acceptable salt thereof.
224. The compound of any one of Embodiments 1-148, wherein LBM is

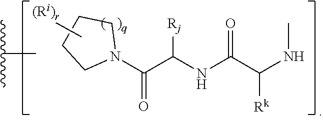

225. The compound of any one of Embodiments 1-148, wherein the compound is of Formula V-1:

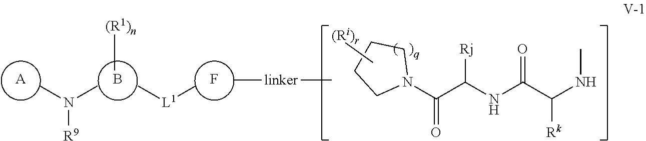

or a pharmaceutically acceptable salt thereof, wherein:
   linker is attached to the bracketed moiety at one Rⁱ or the ring formed when two Rⁱ groups are taken together;
   each Rⁱ that is not the point of attachment of the linker is independently halogen, —R, —C(O)N(R′)₂, or —N(R)C(O)R, or
      two Rⁱ groups, together with the atoms to which they are attached, combine to form an optionally substituted phenyl or 5- to 6-membered ring having 1-3 heteroatoms independently selected from N, O, and S;
   R^{j} is —R, or
      R^{j} combines with one instance of Rⁱ, together with the atoms to which they are attached, to form an optionally substituted 5- to 7-membered ring having 1-2 heteroatoms independently selected from N, O, and S;
   R^{k} is —R;
   r is 1, 2, 3, 4, or 5;
   q is 1 or 2,
   each R′ is independently —R, —C(O)R, or —S(O)₂R, or two R′ attached to the same atom, together with the atom to which they are attached, combine to form an optionally substituted 3- to 16-membered ring having 1-5 heteroatoms independently selected from N, O, and S; and
   each R is independently hydrogen or an optionally substituted group selected from C₁₋₈ aliphatic, C₃₋₁₀ cycloaliphatic, C₁-C₈ heteroaliphatic having 1-3 heteroatoms independently selected from N, O, and S, C₆₋₁₀ aryl, 5- to 10-membered heteroaryl having 1-5 heteroatoms independently selected from N, O, and S, and 3- to 10-membered monocyclic heterocyclyl having 1-5 heteroatoms independently selected from N, O, and S.
226. The compound of any one of Embodiments 1-148, wherein the compound is of Formula V:

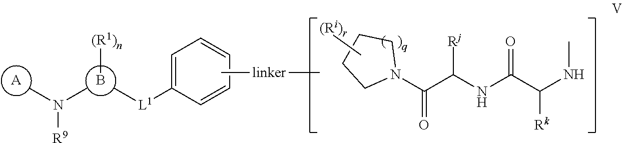

or a pharmaceutically acceptable salt thereof, wherein:
   linker is attached to the bracketed moiety at one Rⁱ or the ring formed when two Rⁱ groups are taken together;
   each Rⁱ that is not the point of attachment of the linker is independently halogen, optionally substituted C₁₋₆ aliphatic, —C(O)N(R)₂, or —N(R)C(O)R, or
   two Rⁱ groups, together with the atoms to which they are attached, combine to form an optionally substituted phenyl or 5- to 6-membered heteroaryl ring having 1-3 heteroatoms independently selected from N, O, and S;
   R^{j} is an optionally substituted group selected from C₁₋₆ aliphatic and C₃₋₇ cycloaliphatic, or
   R^{j} combines with one instance of Rⁱ, together with the atoms to which they are attached, to form an optionally substituted 5- to 7-membered heterocycle having 1-2 heteroatoms independently selected from N, O, and S;
   R^{k} is an optionally substituted C₁₋₆ aliphatic;
   r is 1, 2, 3, 4, or 5; and
   q is 1 or 2.
227. The compound of any one of Embodiments 224-226, wherein at least one Rⁱ is —C(O)N(R)₂.
228. The compound of any one of Embodiments 224-227, wherein two Rⁱ groups, together with the atoms to which they are attached, combine to form an optionally substituted phenyl or 5- to 6-membered heteroaryl ring having 1-3 heteroatoms independently selected from N, O, and S.
229. The compound of any one of Embodiments 224-228, wherein the compound is of Formula VA:

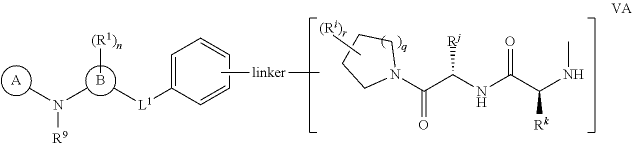

or a pharmaceutically acceptable salt thereof.
230. The compound of Embodiment 229, wherein the compound is of Formula VA-1:

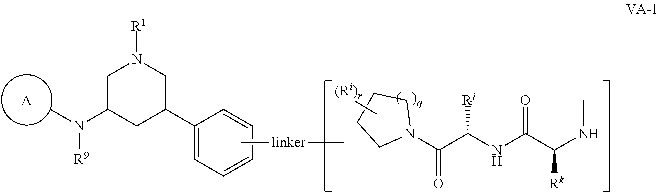

or a pharmaceutically acceptable salt thereof.
231. The compound of Embodiment 229, wherein the compound is of Formula VB:

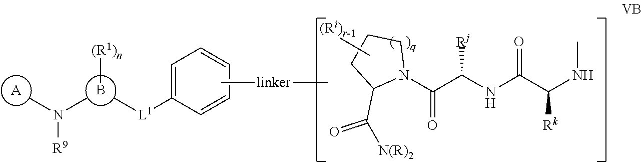

or a pharmaceutically acceptable salt thereof.
232. The compound of Embodiment 231, wherein the compound is of Formula VB-1:

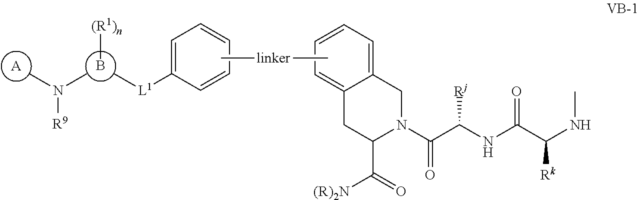

or a pharmaceutically acceptable salt thereof.
233. The compound of any one of Embodiments 1-148, wherein LBM is

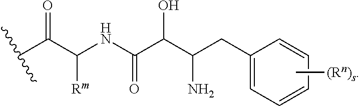

234. The compound of any one of Embodiments 1-148, wherein the compound is of Formula VI-1:

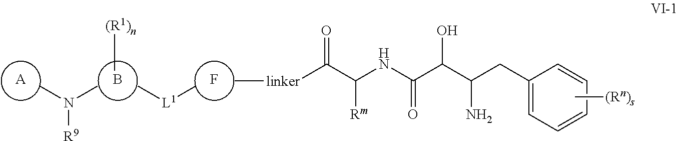

or a pharmaceutically acceptable salt thereof, wherein:
   R^{m} is R;
   each Rⁿ is independently halogen, —CN, —R or —OR;
   s is 0, 1, 2, 3, 4, or 5;
   each R′ is independently —R, —C(O)R, or —S(O)₂R, or two R′ attached to the same atom, together with the atom to which they are attached, combine to form an optionally substituted 3- to 16-membered ring having 1-5 heteroatoms independently selected from N, O, and S; and
   each R is independently hydrogen or an optionally substituted group selected from C₁₋₈ aliphatic, C₃₋₁₀ cycloaliphatic, C₁-C₈ heteroaliphatic having 1-3 heteroatoms independently selected from N, O, and S, C₆₋₁₀ aryl, 5- to 10-membered heteroaryl having 1-5 heteroatoms independently selected from N, O, and S, and 3- to 10-membered monocyclic heterocyclyl having 1-S heteroatoms independently selected from N, O, and S.
235. The compound of any one of Embodiments 1-148, wherein the compound is of Formula VI:

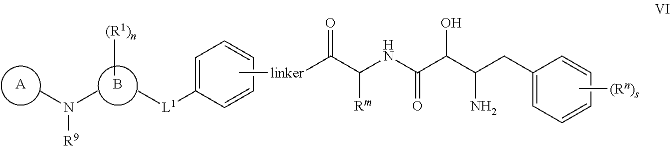

or a pharmaceutically acceptable salt thereof, wherein:
   R^{m} is optionally substituted C₁₋₆ aliphatic;
   each Rⁿ is independently halogen, —OR, —CN, or optionally substituted C₁₋₆ aliphatic; and
   s is 0, 1, 2, 3, 4, or 5.
236. The compound of Embodiment 235, wherein the compound is of Formula VIA:

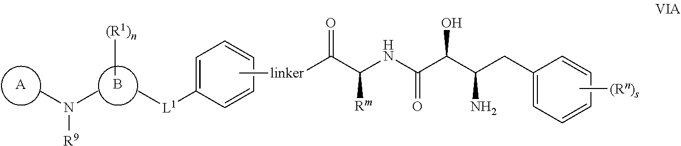

or a pharmaceutically acceptable salt thereof.
237. The compound of Embodiment 236, wherein the compound is of Formula VIA-1:

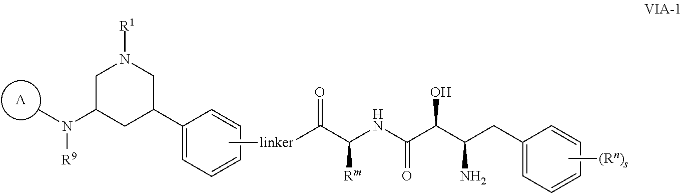

or a pharmaceutically acceptable salt thereof.
238. The compound of any one of Embodiments 1-148, wherein LBM is

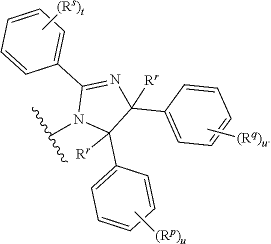

239. The compound of any one of Embodiments 1-148, wherein the compound is of Formula VII-1:

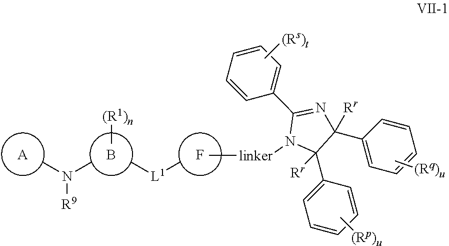

or a pharmaceutically acceptable salt thereof, wherein:
   each R^{p} is independently halogen, —OR, —CN, or optionally substituted C₁₋₆ aliphatic;
   each R^{q} is independently halogen, —OR, —CN, or optionally substituted C₁₋₆ aliphatic;
   each R^{r} is independently hydrogen or optionally substituted C₁₋₆ aliphatic;
   each R^{s} is independently halogen, —OR, —CN, or optionally substituted C₁₋₆ aliphatic;
   t is 0, 1, 2, 3, 4, or 5; and
   each u is independently 0, 1, 2, 3, 4, or 5.
240. The compound of any one of Embodiments 1-148, wherein the compound is of Formula VII:

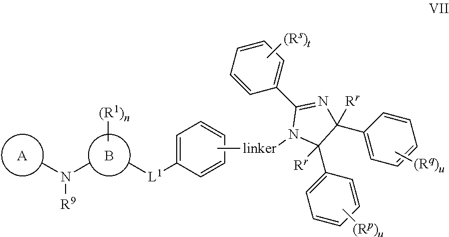

or a pharmaceutically acceptable salt thereof, wherein:
   each R^{p} is independently halogen, —OR, —CN, or optionally substituted C₁₋₆ aliphatic;
   each R^{q} is independently halogen, —OR, —CN, or optionally substituted C₁₋₆ aliphatic;
   each R^{r} is independently hydrogen or optionally substituted C₁₋₆ aliphatic;
   each R^{s} is independently halogen, —OR, —CN, or optionally substituted C₁₋₆ aliphatic;
   t is 0, 1, 2, 3, 4, or 5; and
   each u is independently 0, 1, 2, 3, 4, or 5.
241. The compound of Embodiment 240, wherein the compound is of Formula VIIA:

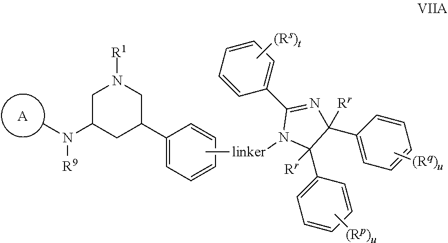

or a pharmaceutically acceptable salt thereof.
242. The compound of any one of Embodiments 1-148, wherein LBM is

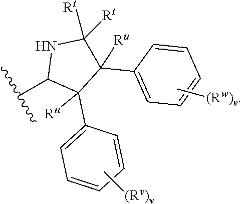

243. The compound of any one of Embodiments 1-148, wherein the compound is of Formula VIII-1:

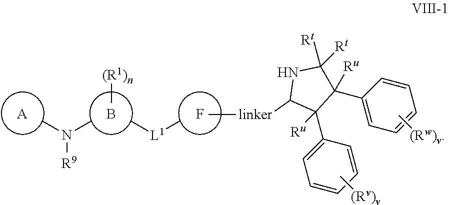

244. The compound of any one of Embodiments 1-148, wherein the compound is of Formula VIII:

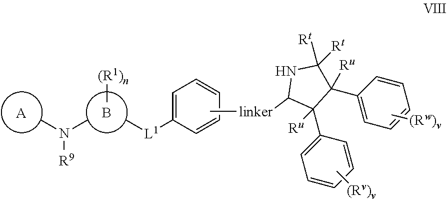

or a pharmaceutically acceptable salt thereof, wherein:
   each R^{t} is independently hydrogen or optionally substituted C₁₋₆ aliphatic, or
   both R^{t} groups, together with the atom to which they are attached, combine to form an optionally substituted 3- to 7-membered cycloaliphatic or heterocycle having 1-2 heteroatoms independently selected from N, O, and S;
   each R^{u} is independently hydrogen, halogen, —CN, or optionally substituted C₁₋₆ aliphatic;
   each R^{v} is independently halogen, —OR, —CN, or optionally substituted C₁₋₆ aliphatic, or
   one instance of R^{u} and R^{v}, together with the atoms to which they are attached combine to form an optionally substituted 3- to 7-membered cycloaliphatic or heterocycle having 1-2 heteroatoms independently selected from N, O, and S;
   each R^{w} is independently halogen, —OR, —CN, or optionally substituted C₁₋₆ aliphatic, or one instance of R^{u} and R^{w}, together with the atoms to which they are attached, combine to form an optionally substituted 3- to 7-membered cycloaliphatic or heterocycle having 1-2 heteroatoms independently selected from N, O, and S;
   each v is independently 0, 1, 2, 3, 4, or 5.
245. The compound of any one of Embodiments 242-244, wherein the compound is of Formula VIIIA:

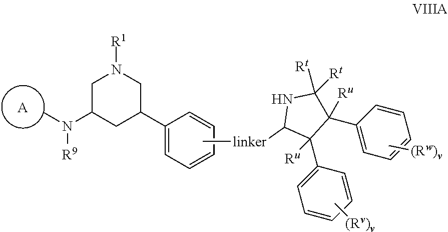

or a pharmaceutically acceptable salt thereof.
246. The compound of any one of Embodiments 1-4, wherein PBM is

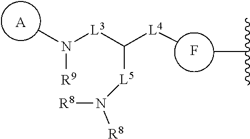

247. A compound of Formula IX-1:

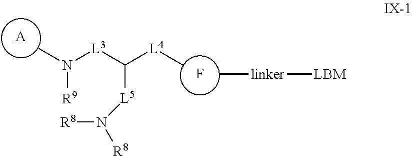

or a pharmaceutically acceptable salt thereof, wherein:
   Ring A is selected from:

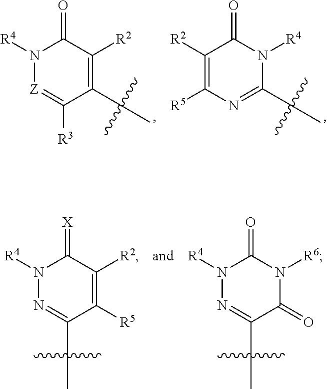

Z is N or CR³;
R² is halogen, —CN, or —R;
each R³ is independently halogen, —CN, or —R;
R⁴ is —R′;
R⁵ is halogen, —CN, or —R, or
   R² and R⁵, together with the atoms to which they are attached, combine to form an optionally substituted 5- to 6-membered ring having 0-2 heteroatoms independently selected from N, O, and S;
R⁶ is —R′;
X is O or NR⁷;
R⁷ is —R′, or
   R⁴ and R⁷, together with the atoms to which they are attached, combine to form an optionally substituted 5- to 6-membered ring having 2-3 heteroatoms independently selected from N, O, and S;
R⁹ is —R′;
L³, L⁴, and L⁵ are each independently a covalent bond or an optionally substituted bivalent C₁₋₆ hydrocarbon chain;
each R⁸ is independently —R′;
Ring F is an optionally substituted 3- to 16-membered ring having 0-6 heteroatoms independently selected from N, O, and S;
linker is a covalent bond or an optionally substituted, bivalent, straight or branched, saturated or linker is a covalent bond or an optionally substituted bivalent C₁-C₂₀ hydrocarbon chain, wherein one or more methylene units are optionally and independently replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —S(O)—, —SO₂—, —SO—N(R)—, —N(R)SO₂—, or -Cy-;
each Cy is independently an optionally substituted, 3- to 16-membered bivalent ring having 0-6 heteroatoms independently selected from N, O, and S;
LBM is an E3 ubiquitin ligase binding moiety;
each R′ is independently —R, —C(O)R, or —S(O)₂R, or two R′ attached to the same atom, together with the atom to which they are attached, combine to form an optionally substituted 3- to 16-membered ring having 1-5 heteroatoms independently selected from N, O, and S; and
each R is independently hydrogen or an optionally substituted group selected from C₁₋₈ aliphatic, C₃₋₁₀ cycloaliphatic, C₁-C₈ heteroaliphatic having 1-3 heteroatoms independently selected from N, O, and S, C₆₋₁₀ aryl, 5- to 10-membered heteroaryl having 1-5 heteroatoms independently selected from N, O, and S, and 3- to 10-membered monocyclic heterocyclyl having 1-5 heteroatoms independently selected from N, O, and S.
248. The compound of any one of Embodiments 246-247, wherein Ring F is an optionally substituted 3- to 10-membered ring having 0-4 heteroatoms independently selected from N, O, and S.
249. The compound of any one of Embodiments 246-247, wherein Ring F is an optionally substituted 5-6 membered ring having 0-4 heteroatoms independently selected from N, O, and S.
250. The compound of any one of Embodiments 246-247, wherein Ring F is an optionally substituted 6-membered ring.
251. The compound of any one of Embodiments 246-248, wherein Ring F is aromatic.
252. The compound of any one of Embodiments 246-251, wherein Ring F is an optionally substituted phenyl ring.
253. The compound of any one of Embodiments 246-251, wherein Ring F is optionally substituted 1,4-phenylene.
254. The compound of any one of Embodiments 246-251, wherein Ring F is 1,4-phenylene.
255. A compound of Formula IX:

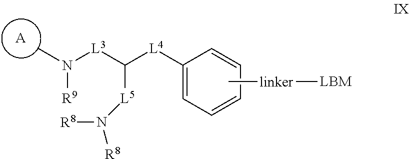

or a pharmaceutically acceptable salt thereof, wherein:
   Ring A is selected from:

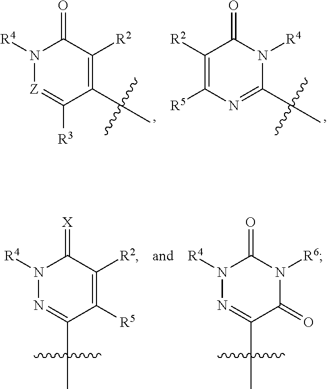

L³, L⁴, and L⁵ are each independently a covalent bond or an optionally substituted bivalent C₁₋₆ straight or branched hydrocarbon chain;
Z is N or CR³;
R² is hydrogen, halogen, —CN, optionally substituted C₁₋₆ aliphatic, or optionally substituted C₃₋₆ cycloaliphatic;
each R³ is independently hydrogen, halogen, optionally substituted C₁₋₆ aliphatic, or optionally substituted C₃₋₆ cycloaliphatic;
R⁴ is hydrogen, optionally substituted C₁₋₆ aliphatic, or optionally substituted C₃₋₆ cycloaliphatic;
R⁵ is hydrogen, halogen, optionally substituted C₁₋₆ aliphatic, or optionally substituted C₃₋₆ cycloaliphatic, or
R² and R⁵, together with the atoms to which they are attached, combine to form an optionally substituted 5- to 6-membered aromatic ring having 0-2 heteroatoms independently selected from N, O, and S;
R⁶ is hydrogen, optionally substituted C₁₋₆ aliphatic, or optionally substituted C₃₋₆ cycloaliphatic;
X is O or NR⁷;
R⁷ is hydrogen or optionally substituted C₁₋₆ aliphatic, or
R⁴ and R⁷, together with the atoms to which they are attached, combine to form an optionally substituted 5- to 6-membered aromatic ring having 2-3 heteroatoms independently selected from N, O, and S;
each R⁸ is independently hydrogen or optionally substituted C₁₋₆ aliphatic;
R⁹ is hydrogen or optionally substituted C₁₋₆ aliphatic;
linker is a covalent bond or an optionally substituted, bivalent, straight or branched, saturated or unsaturated C₁-C₂₀ hydrocarbon chain, wherein one or more methylene units are optionally and independently replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —SO₂—, —SO₂N(R)—, —N(R)SO₂—, or -Cy-;
each Cy is independently an optionally substituted, mono- or multicyclic, 3- to 16-membered bivalent ring system, wherein the ring system is fully saturated, partially saturated, or aromatic, and the ring system contains 0-6 heteroatoms independently selected from N, O, and S;
each R is independently hydrogen or an optionally substituted group selected from C₁₋₆ aliphatic, phenyl, C₃₋₇ cycloaliphatic, 5- to 6-membered monocyclic heteroaryl having 1-4 heteroatoms independently selected from N, O, and S, or a 3- to 7-membered monocyclic heterocyclyl having 1-2 heteroatoms independently selected from N, O, and S; and
LBM is an E3 ubiquitin ligase binding moiety.
256. The compound of any one of Embodiments 246-255, wherein R² is hydrogen, halogen, optionally substituted C₁₋₆ aliphatic, or optionally substituted C₃₋₆ cycloaliphatic.
257. The compound of any one of Embodiments 246-255, wherein R² is hydrogen.
258 The compound of any one of Embodiments 246-255, wherein R² is halogen.
259 The compound of any one of Embodiments 246-255, wherein R² is —Cl.
260 The compound of any one of Embodiments 246-255, wherein R² is —Br.
261. The compound of any one of Embodiments 246-255, wherein R² is optionally substituted C₁₋₆ aliphatic, or optionally substituted C₃₋₆ cycloaliphatic.
262. The compound of any one of Embodiments 246-255, wherein R³ is hydrogen.
263. The compound of any one of Embodiments 246-262, wherein Ring A is

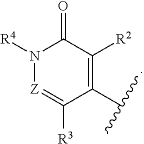

264. The compound of Embodiment 263, wherein Z is N.
265. The compound of Embodiment 263, wherein Z is CR³.
266. The compound of any one of Embodiments 263-265, wherein R⁴ is optionally substituted C₁₋₆ aliphatic.
267. The compound of any one of Embodiments 263-265, wherein R⁴ is optionally substituted C₁₋₆ alkyl.
268. The compound of any one of Embodiments 263-265, wherein R⁴ is C₁₋₆ alkyl.
269. The compound of any one of Embodiments 263-265, wherein R⁴ is methyl.
270. The compound of Embodiment 263, wherein Ring A is selected from

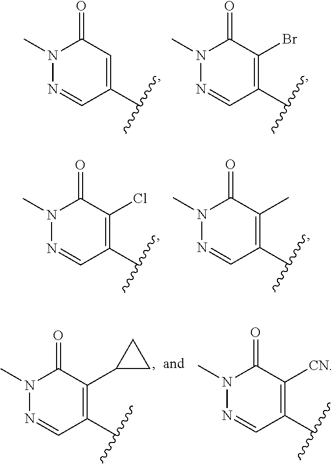

271. The compound of Embodiment 263, wherein Ring A is selected from

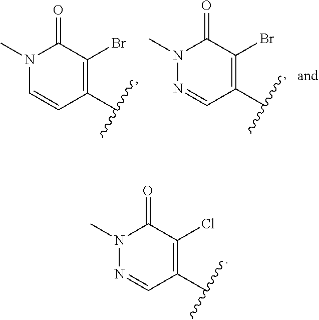

272. The compound of Embodiment 263, wherein Ring A is

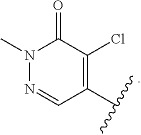 [SMILES: *c1cnn(C)c(=O)c1Cl]

273. The compound of Embodiment 263, wherein Ring A is

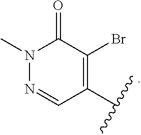 [SMILES: *c1cnn(C)c(=O)c1Br]

274. The compound of any one of Embodiments 246-262, wherein Ring A is

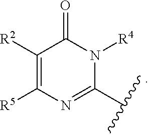

275. The compound of any one of Embodiments 246-262, wherein Ring A is

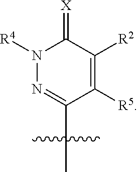

276. The compound of Embodiment 275, wherein X is O.
277. The compound of Embodiment 275, wherein X is NR⁷.
278. The compound of Embodiment 277 wherein R⁴ and R⁷, together with the atoms to which they are attached, combine to form an optionally substituted 5- to 6-membered ring having 2-3 heteroatoms independently selected from N, O, and S.
279. The compound of Embodiment 278, wherein the formed ring is aromatic.
280. The compound of any one of Embodiments 274-279, wherein R² and R⁵, together with the atoms to which they are attached, combine to form an optionally substituted 5- to 6-membered ring having 0-2 heteroatoms independently selected from N, O, and S.
281. The compound of Embodiment 280, wherein the formed ring is aromatic.
282. The compound of any one of Embodiments 246-262, wherein Ring A is

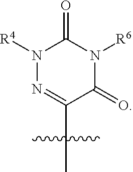

283. The compound of Embodiment 282, wherein R⁶ is optionally substituted C₁₋₆ aliphatic.
284. The compound of any one of Embodiments 274-283, wherein R⁴ is optionally substituted C₁₋₆ aliphatic.
285. The compound of any one of Embodiments 274-283, wherein R⁴ is optionally substituted C₁₋₆ alkyl.
286. The compound of any one of Embodiments 274-283, wherein R⁴ is C₁₋₆ alkyl.
287. The compound of any one of Embodiments 274-283, wherein R⁴ is methyl.
288. The compound of Embodiment 274, wherein Ring A is

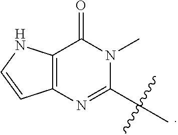 [SMILES: *c1nc2cc[nH]c2c(=O)n1C]

289. The compound of Embodiment 275, wherein Ring A is selected from

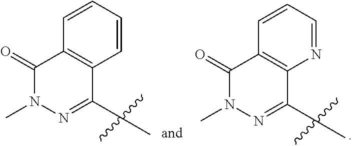 [SMILES: *c1nn(C)c(=O)c2ccccc12 ; *c1nn(C)c(=O)c2cccnc12]

290. The compound of Embodiment 275, wherein Ring A is

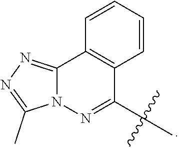 [SMILES: *c1nn2c(C)nnc2c2ccccc12]

291. The compound of Embodiment 275, wherein Ring A is

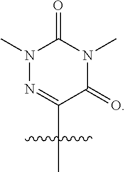

292. The compound of any one of Embodiments 246-291, wherein R⁹ is hydrogen.
293. The compound of any one of Embodiments 246-292, wherein L³ is a covalent bond.
294. The compound of any one of Embodiments 246-292, wherein L³ is an optionally substituted bivalent C₁₋₃ straight or branched hydrocarbon chain.
295. The compound of any one of Embodiments 246-294, wherein L⁴ is a covalent bond.
296. The compound of any one of Embodiments 246-294, wherein L⁴ is an optionally substituted bivalent C₁₋₃ straight or branched hydrocarbon chain.
297. The compound of any one of Embodiments 246-296, wherein L⁴ is a covalent bond.
298. The compound of any one of Embodiments 246-296, wherein L⁴ is an optionally substituted bivalent C₁₋₃ straight or branched hydrocarbon chain.
299. The compound of any one of Embodiments 246-298, wherein each R⁸ is optionally substituted C₁₋₆ alkyl.
300. The compound of any one of Embodiments 246-298, wherein each R⁸ is C₁-6 alkyl.
301. The compound of any one of Embodiments 246-300, wherein the linker is less than 14 atoms in length.
302. The compound of any one of Embodiments 246-300, wherein the linker is less than 11 atoms in length.
303. The compound of any one of Embodiments 246-300, wherein the linker is less than 11 atoms in length.
304. The compound of any one of Embodiments 246-300, wherein the shortest-path length of linker is about or no more than 14 atoms.
305. The compound of any one of Embodiments 246-300, wherein the shortest-path length of linker is about or no more than 10 atoms.
306. The compound of any one of Embodiments 246-300, wherein the number of non-ring consecutive sp³ atoms in the shortest path chain of linker is about or no more than about 5, 4, 3, 2 or 1.
307. The compound of any one of Embodiments 246-300, wherein the number of non-ring consecutive sp³ atoms in the shortest path chain of linker is about or no more than about 2.
308. The compound of any one of Embodiments 246-300, wherein the number of bonds between two non-ring sp³ atoms in the shortest path chain of linker is about or no more than 5, 4, 3, 2 or 1.
309. The compound of any one of Embodiments 246-300, wherein the number of bonds between two non-ring sp³ atoms in the shortest path chain of linker is about or no more than 1.
310. The compound of any one of Embodiments 246-300, wherein the number of non-ring sp³ C, O, and S atoms in the shortest path chain of linker is about or no more than about 5, 4, 3, 2 or 1.
311. The compound of any one of Embodiments 246-300, wherein the number of non-ring sp³ C, O, and S atoms in the shortest path chain of linker is about or no more than about 1.
312. The compound of any one of Embodiments 246-300, wherein the number of non-ring sp³ atoms in the shortest path chain of linker is about or no more than about 5, 4, 3, 2 or 1.
313. The compound of any one of Embodiments 246-300, wherein the number of non-ring sp³ atoms in the shortest path chain of linker is about or no more than about 1.
314. The compound of any one of Embodiments 246-300, wherein the number of non-ring atoms in the shortest path chain of linker is about or no more than about 5, 4, 3, 2 or 1.
315. The compound of any one of Embodiments 246-300, wherein the number of non-ring atoms in the shortest path chain of linker is about or no more than about 1.
316. The compound of any one of Embodiments 246-300, wherein linker is an optionally substituted, bivalent, straight or branched, saturated or unsaturated C₁-C₂₀ hydrocarbon chain, wherein one or more methylene units are optionally and independently replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —SO₂—, —SO₂N(R)—, —N(R)SO₂—, or -Cy-.
317. The compound of any one of Embodiments 246-300, wherein linker is an optionally substituted, bivalent, straight or branched, saturated or unsaturated C₁-C₂₀ hydrocarbon chain, wherein one or more methylene units are optionally and independently replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —SO₂—, —SO₂N(R)—, —N(R)SO₂, or -Cy-.
318. The compound of any one of Embodiments 246-300, wherein linker is an optionally substituted, bivalent, straight or branched, saturated or unsaturated C₁-C₂₀ hydrocarbon chain, wherein at least one methylene unit is replaced by —O—, —N(R)—, —C(O)—, —OC(O)—, —C(O)O—, —C(O)N(R)—, —N(R)C(O)—, or -Cy-.
319. The compound of any one of Embodiments 246-300, wherein linker is an optionally substituted, bivalent, straight or branched, saturated or unsaturated C₁-C₂₀ hydrocarbon chain, wherein at least two methylene units are replaced by —O—, —N(R)—, —C(O)—, —OC(O)—, —C(O)O—, —C(O)N(R)—, —N(R)C(O)—, or -Cy-.
320. The compound of any one of Embodiments 246-300, wherein a methylene unit is replaced by —C(O)—.
321. The compound of any one of Embodiments 246-300, wherein a methylene unit is replaced by -Cy-.
322. The compound of Embodiment 321, wherein -Cy- is in a shortest path chain.
323. The compound of any one of Embodiments 246-322, wherein linker is an optionally substituted, bivalent, straight or branched, saturated or unsaturated C₁-C₂₀ hydrocarbon chain, wherein at least one methylene unit is replaced by —C(O)N(R)—.
324. The compound of any one of Embodiments 246-322, wherein linker comprises at least one triple bond.
325. The compound of any one of Embodiments 246-322, wherein linker is an optionally substituted, bivalent, straight or branched, saturated or unsaturated C₁-C₂₀ hydrocarbon chain, wherein at least one methylene unit is replaced by -Cy-.
326. The compound of any one of Embodiments 246-322, wherein linker is:

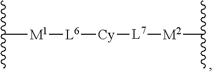

wherein:
   M¹ and M² are each independently absent, —CH₂—, —O—, —N(R)—, —C(O)—, —OC(O)—, —C(O)O—, —C(O)N(R)—, or —N(R)C(O)—; and
   L⁶ and L⁷ are each independently a covalent bond or an optionally substituted, bivalent, straight or branched, saturated or unsaturated C₁-C₁₀ hydrocarbon chain, wherein one or more methylene units are optionally and independently replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)—, —OC(O)N(R)—, —N(R)(O)S—, —SC(O)N(R)—, —N(R)C(NR)N(R)—, —SO₂—, —SO₂N(R)—, —N(R)SO₂—, or -Cy-.
327. The compound of any one of Embodiments 246-322, wherein linker is selected from

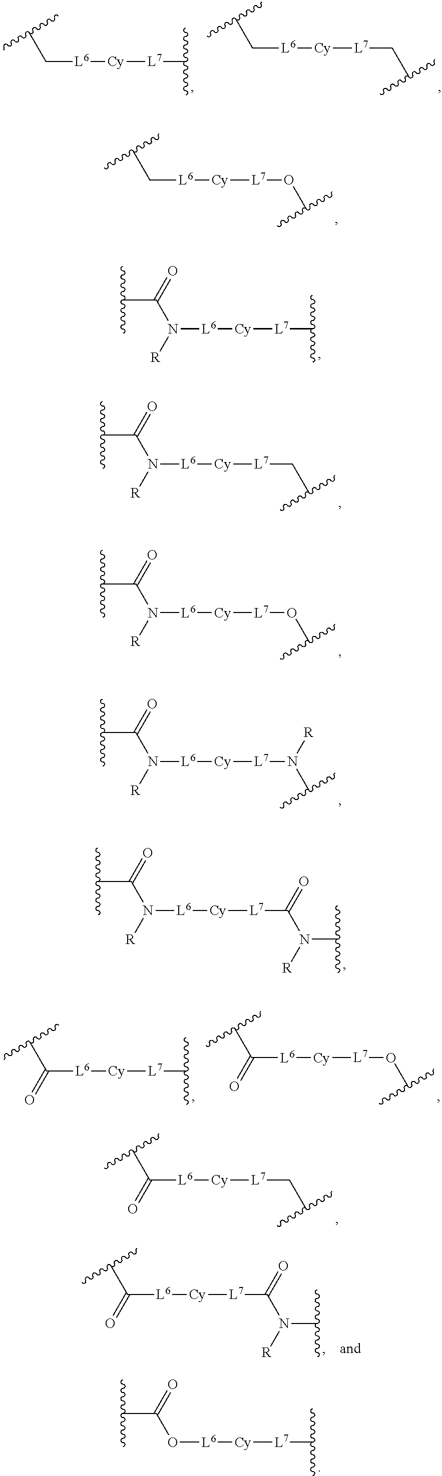

328. The compound of any one of Embodiments 246-322, wherein -Cy- is monocyclic.
329. The compound of Embodiment 328, wherein -Cy- is 3-10 membered.
330. The compound of any one of Embodiments 328-329, wherein -Cy- is 4-, 5- or 6-membered.
331. The compound of any one of Embodiments 328-330, wherein -Cy- is saturated.
332 The compound of any one of Embodiments 328-331, wherein -Cy- is partially unsaturated.
333. The compound of any one of Embodiments 328-332, wherein -Cy- has 1-2 heteroatoms independently selected from N, O, and S.
334. The compound of any one of Embodiments 328-333, wherein -Cy- has at least one nitrogen atom.
335. The compound of any one of Embodiments 246-322, wherein -Cy- is polycyclic.
336 The compound of any one of Embodiments 246-322, wherein -Cy- is bicyclic.
337. The compound of any one of Embodiments 335-336, wherein -Cy- is 6-16 membered.
338 The compound of any one of Embodiments 335-337, wherein -Cy- is 7-12 membered.
339 The compound of any one of Embodiments 335-338, wherein -Cy- is 7-, 8-, 9-, 10- or 11-membered.
340. The compound of any one of Embodiments 335-338, wherein -Cy- is 11-membered.
341. The compound of any one of Embodiments 335-340, wherein each monocyclic ring unit is independently 4-7 membered.
342. The compound of any one of Embodiments 335-341, wherein each monocyclic ring unit is independently 4-6 membered.
343. The compound of any one of Embodiments 335-342, wherein a monocyclic ring unit is saturated.
344. The compound of any one of Embodiments 335-343, wherein a monocyclic ring unit has 1-2 heteroatoms independently selected from N, O, and S.
345. The compound of any one of Embodiments 335-344, wherein each monocyclic ring unit independently has 1-2 heteroatoms independently selected from N, O, and S.
346. The compound of any one of Embodiments 335-345, wherein each monocyclic ring unit independently has at least one nitrogen atom.
347. The compound of any one of Embodiments 335-346, wherein -Cy- is saturated.
348. The compound of any one of Embodiments 335-346, wherein a monocyclic ring unit is partially unsaturated.
349 The compound of any one of Embodiments 335-348, wherein -Cy- is spiro.
350. The compound of any one of Embodiments 246-349, wherein linker comprises one or more basic nitrogen.
351. The compound of any one of Embodiments 246-349, wherein -Cy- comprises one or more basic nitrogen.
352. The compound of any one of Embodiments 246-349, wherein -Cy- is a diamine ring.
353. The compound of any one of Embodiments 246-327, wherein Cy is optionally substituted phenyl or 5- to 6-membered heteroaryl having 1-4 heteroatoms independently selected from N, O, and S.
354. The compound of any one of Embodiments 246-327, wherein Cy is optionally substituted monocyclic 5- to 6-membered heterocyclyl having 1-2 heteroatoms independently selected from N, O, and S.
355. The compound of any one of Embodiments 246-327, wherein Cy is optionally substituted bicyclic 6- to 11-membered heterocyclyl having 1-3 heteroatoms independently selected from N, O, and S.
356. The compound of any one of Embodiments 246-327, wherein Cy is a spirocyclic 6- to 7-membered heterocyclyl having 1-3 heteroatoms independently selected from N, O, and S.
357. The compound of any one of Embodiments 246-356, wherein -Cy- is substituted.
358. The compound of any one of Embodiments 246-356, wherein -Cy- is unsubstituted.
359. The compound of any one of Embodiments 246-356, wherein linker is selected from

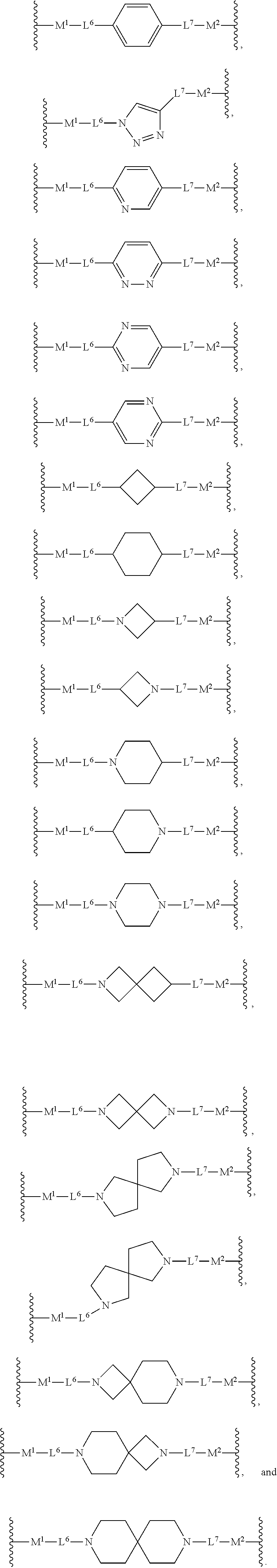

360. The compound of any one of Embodiments 246-325, wherein linker is -M¹-L⁶-L⁷-M²-.
361. The compound of any one of Embodiments 326-360, wherein M¹ is a covalent bond.
362. The compound of any one of Embodiments 326-360, wherein M¹ is optionally substituted —CH₂—.
363. The compound of any one of Embodiments 326-360, wherein M¹ is —CH₂—.
364. The compound of any one of Embodiments 326-360, wherein M¹ is —C(O)—.
365. The compound of any one of Embodiments 326-364, wherein M¹ is bonded to PBM.
366 The compound of any one of Embodiments 326-365, wherein L⁶ is a covalent bond.
367. The compound of any one of Embodiments 326-365, wherein L⁶ is optionally substituted —CH₂—.
368 The compound of any one of Embodiments 326-367, wherein L⁷ is a covalent bond.
369 The compound of any one of Embodiments 326-367, wherein L⁷ is —O—.
370 The compound of any one of Embodiments 326-369, wherein M² is a covalent bond.
371. The compound of any one of Embodiments 326-369, wherein M² is —O—.
372. The compound of any one of the preceding Embodiments, wherein each R is independently hydrogen or optionally substituted C₁₋₆ alkyl.
373. The compound of any one of Embodiments 246-300, wherein linker is a linker of Embodiment 138.
374. The compound of any one of Embodiments 246-300, wherein linker is a linker of Embodiment 139.
375. The compound of any one of Embodiments 246-300, wherein linker is a linker of Embodiment 140.
376 The compound of any one of Embodiments 246-375, wherein LBM is a CRBN binding moiety.
377. The compound of any one of Embodiments 246-375, wherein LBM is a VHL binding moiety.
378. The compound of any one of Embodiments 246-375, wherein LBM is an IAP binding moiety.
379 The compound of any one of Embodiments 246-375, wherein LBM is a MDM2 binding moiety.
380 The compound of any one of Embodiments 246-375, wherein the compound is of Formula IXA, IXB, IXC, or IXD:

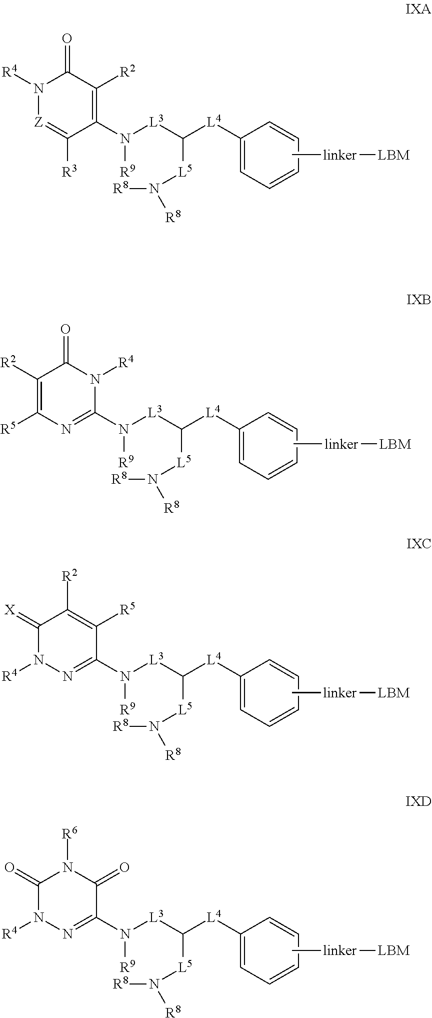

or a pharmaceutically acceptable salt thereof.
381. The compound of any one of Embodiments 246-375, wherein LBM is

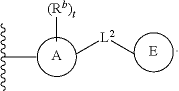

382. The compound of any one of Embodiments 246-375, wherein the compound is of Formula X-1:

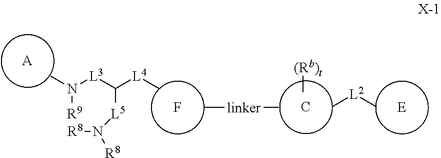

or a pharmaceutically acceptable salt thereof, wherein:
   each R^{b} is independently halogen, —CN, —R′ or —OR;
   t is 0, 1, 2, 3, or 4;
   L² is a covalent bond or an optionally substituted C₁₋₃ hydrocarbon chain, wherein one or more methylene units are optionally and independently replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —S(O)—, —SO₂—, —SO₂N(R)—, —N(R)SO₂—, or -Cy-;
   Ring E is

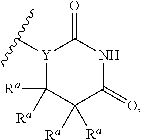

or an optionally substituted group selected from

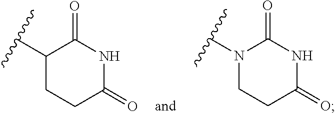 [SMILES: *C1CCC(=O)NC1=O ; "and" ; *N1CCC(=O)NC1=O]

Y is Nor CH; and
each R^{a} is independently halogen, —CN or —R′.
383. The compound of Embodiment 382, wherein t is 1.
384. The compound of Embodiment 382, wherein t is 0.
385. The compound of any one of Embodiments 246-375, wherein the compound is of Formula X-3:

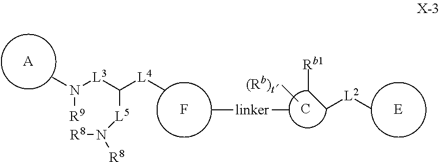

or a pharmaceutically acceptable salt thereof, wherein:
   each R^{b} and R^{b1} is independently halogen, —CN, —R′ or —OR;
   t′ is 0, 1, 2, or 3;
   L² is a covalent bond or an optionally substituted C₁₋₃ hydrocarbon chain, wherein one or more methylene units are optionally and independently replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —S(O)—, —SO₂—, —SO₂N(R)—, —N(R)SO₂—, or -Cy-;
   Ring E is

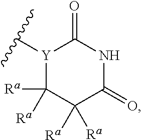

or an optionally substituted group selected from

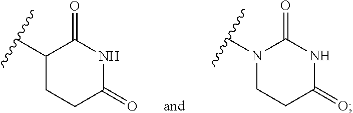 [SMILES: *C1CCC(=O)NC1=O ; "and" ; *N1CCC(=O)NC1=O]

Y is Nor CH; and
each R^{a} is independently halogen, —CN or —R′.
386. The compound of Embodiment 385, wherein t′ is 0.
387. The compound of any one of Embodiments 385-386, wherein R^{b1} is not —H.
388. The compound of any one of Embodiments 385-386, wherein R^{b1} is optionally substituted C₁₋₆ aliphatic.
389. The compound of any one of Embodiments 385-386, wherein R^{b1} is optionally substituted C₁₋₆ alkyl.
390. The compound of any one of Embodiments 385-386, wherein R^{b1} is C₁₋₆ alkyl.
391. The compound of any one of Embodiments 385-386, wherein R^{b1} is C₁₋₆ haloalkyl.
392. The compound of any one of Embodiments 385-386, wherein R^{b1} is —OR wherein R is optionally substituted C₁₋₆ aliphatic.
393. The compound of any one of Embodiments 385-386, wherein R^{b1} is —OR wherein R is optionally substituted C₁₋₆ alkyl.
394. The compound of any one of Embodiments 385-386, wherein R^{b1} is —OR wherein R is C₁₋₆ alkyl.
395 The compound of any one of Embodiments 385-386, wherein R^{b1} is —OR wherein R is C₁₋₆ haloalkyl.
396. The compound of any one of Embodiments 385-386, wherein R^{b1} is halogen.
397. The compound of any one of Embodiments 385-386, wherein R^{b1} is —OCF₃.
398. The compound of any one of Embodiments 385-386, wherein R^{b1} is —OCH₃.
399. The compound of any one of Embodiments 385-386, wherein R^{b1} is —CF₃.
400. The compound of any one of Embodiments 385-386, wherein R^{b1} is —CH₃.
401. The compound of any one of Embodiments 385-386, wherein R^{b1} is —F.
402. The compound of any one of Embodiments 246-401, wherein Ring C is aromatic.
403. The compound of Embodiment 402, wherein Ring C is an optionally substituted phenyl ring.
404. The compound of Embodiment 402, wherein Ring C is phenyl ring.
405. The compound of Embodiment 402, wherein Ring C is optionally substituted

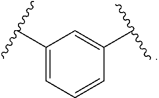 [SMILES: *c1cccc(*)c1]

406. The compound of Embodiment 402, wherein Ring C is optionally substituted

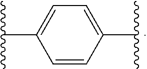 [SMILES: *c1ccc(*)cc1]

407. The compound of Embodiment 402, wherein Ring C is an optionally substituted 5-membered heteroaryl ring having 1-2 heteroatoms independently selected from N, O, and S.
408. The compound of Embodiment 402, wherein Ring C is an optionally substituted 6-membered heteroaryl ring having 1-2 heteroatoms independently selected from N, O, and S.
409. The compound of Embodiment 402, wherein Ring C is an optionally substituted 9-membered bicyclic heteroaryl ring having 1-4 heteroatoms independently selected from N, O, and S.
410. The compound of Embodiment 402, wherein Ring C is an optionally substituted 10-membered bicyclic heteroaryl ring having 1-4 heteroatoms independently selected from N, O, and S.
411. The compound of any one of Embodiments 246-410, wherein Ring E is

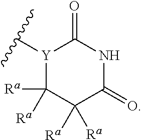

412. The compound of any one of Embodiments 246-410, wherein Ring E is an optionally substituted

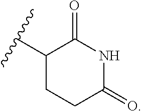 [SMILES: *C1CCC(=O)NC1=O]

413. The compound of any one of Embodiments 246-410, wherein Ring E is an optionally substituted

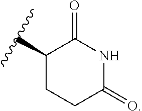 [SMILES: *[C@@H]1CCC(=O)NC1=O]

414. The compound of any one of Embodiments 246-410, wherein Ring E is an optionally substituted

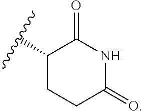 [SMILES: *[C@H]1CCC(=O)NC1=O]

415. The compound of any one of Embodiments 246-410, wherein Ring E is optionally substituted

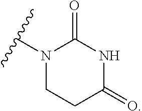 [SMILES: *N1CCC(=O)NC1=O]

416. The compound of any one of Embodiments 246-410, wherein Ring E is

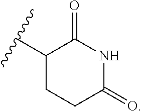 [SMILES: *C1CCC(=O)NC1=O]

417. The compound of any one of Embodiments 246-410, wherein Ring E is

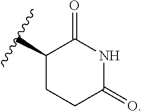 [SMILES: *[C@@H]1CCC(=O)NC1=O]

418. The compound of any one of Embodiments 246-410, wherein Ring Bis

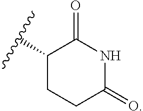 [SMILES: *[C@H]1CCC(=O)NC1=O]

419. The compound of any one of Embodiments 246-410, wherein Ring E is

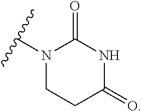 [SMILES: *N1CCC(=O)NC1=O]

420. The compound of any one of Embodiments 246-375, wherein the compound is of Formula X:

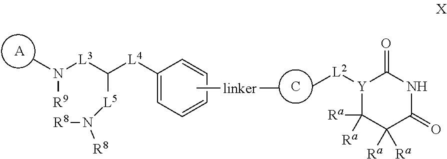

or a pharmaceutically acceptable salt thereof, wherein:
   Ring C is an optionally substituted, mono- or multicyclic, 3- to 16-membered bivalent ring system, wherein the ring system is fully saturated, partially saturated, or aromatic, and the ring system contains 0-6 heteroatoms independently selected from N, O, and S;
   each R^{a} is independently hydrogen or an optionally substituted C₁₋₆ aliphatic, or two R^{a} groups, together with the atom to which they are attached, combine to form a 3- to 6-membered saturated or partially unsaturated ring;
   L² is a covalent bond or a straight or branched C₁₋₃ hydrocarbon chain wherein one methylene is optionally replaced with —O—, —S—, —N(R)—, —SO₂—, —C(O)N(R)—, or —N(R)C(O)—; and
   Y is N or CH.
421. The compound of Embodiment 420, wherein Ring C is optionally substituted phenyl, C₅₋₆ cycloaliphatic, 5- to 6-membered heteroaryl having 1-4 heteroatoms independently selected from N, O, and S, 5- to 6-membered heterocyclyl having 1-2 heteroatoms independently selected from N, O, and S, or 9- to 10-membered bicyclic heteroaryl having 1-4 heteroatoms independently selected from N, O, and S.
422. The compound of Embodiment 420, wherein Ring C is optionally substituted phenyl or 5- to 6-membered heteroaryl having 1-4 heteroatoms independently selected from N, O, and S.
423. The compound of Embodiment 420, wherein Ring C is

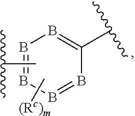

wherein:
   each B is independently selected from N, C, and CH, provided that no more than two B are N;
   each R^{c} is independently selected from halogen, —OR, —N(R)₂, —CN, and optionally substituted C₁₋₆ aliphatic; and
m is 0, 1, 2, or 3.
424. The compound of Embodiment 420, wherein Ring C is

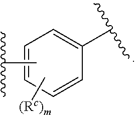

425. The compound of Embodiment 420, wherein the compound is of Formula XB:

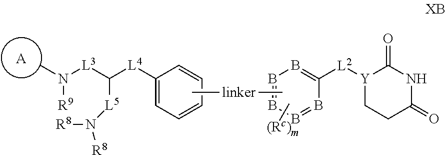

or a pharmaceutically acceptable salt thereof.
426. The compound of Embodiment 420, wherein Ring C is optionally substituted C₃-C₇ cycloaliphatic or 3- to 7-membered heterocyclyl having 1-2 heteroatoms independently selected from N, O, and S.
427. The compound of Embodiment 420, wherein Ring C is optionally substituted 9- to 10-membered bicyclic heteroaryl having 1-4 heteroatoms independently selected from N, O, and S.
428. The compound of Embodiment 427, wherein Ring C is:

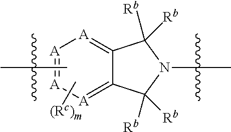

wherein:
   each A is independently N, C, or CH, provided that no more than two A groups are N;
   each R^{b} is hydrogen, or two R^{b} groups, on the same carbon, are taken together to form an oxo or combine to form a 3- to 6-membered saturated or partially unsaturated ring;
   each R^{c} is independently selected from halogen, —OR, —N(R)₂, —CN, and optionally substituted C₁₋₆ aliphatic; and
   m is 0, 1, 2, or 3.
429. The compound of Embodiment 428, wherein Ring C is:

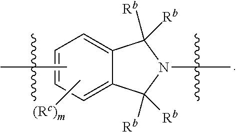

430. The compound of any one of Embodiments 381-434, wherein L² is a covalent bond.
431. The compound of any one of Embodiments 381-434, wherein L² is optionally substituted —CH₂—.
432. The compound of any one of Embodiments 381-434, wherein L² is —CH₂—.
433. The compound of any one of Embodiments 381-434, wherein L² is —O—.
434. The compound of any one of Embodiments 381-434, wherein L² is —S—.
435. The compound of any one of Embodiments 381-434, wherein L² is —C(O)N(R)—, or —N(R)C(O)—.
436. The compound of any one of Embodiments 381-434, wherein L² is —C(O)NH—, or —NHC(O)—.
437. The compound of any one of Embodiments 381-434, wherein L² is —N(R)—.
438. The compound of any one of Embodiments 381-434, wherein L² is —NH—.
439. The compound of any one of Embodiments 381-434, wherein L² is -Cy-.
440. The compound of any one of Embodiments 381-434, wherein L² is -Cy-, wherein -Cy- is aromatic.
441. The compound of any one of Embodiments 420-440, wherein Y is N.
442. The compound of any one of Embodiments 420-440, wherein Y is CH.
443. The compound of any one of Embodiments 420-442, wherein each R^{c} is independently selected from halogen, —O(C₁₋₆ alkyl), C₁₋₆ alkyl, and C₁₋₆ haloalkyl.
444. The compound of any one of Embodiments 381-429, wherein the compound is of Formula XA:

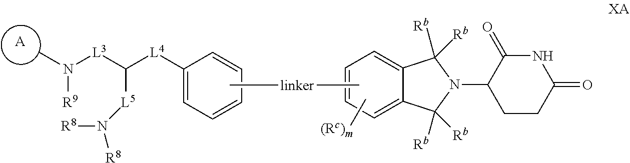

or a pharmaceutically acceptable salt thereof, wherein:
   each R^{b} is hydrogen, or two R^{b} groups, on the same carbon, are taken together to form an oxo or combine to form a 3- to 6-membered saturated or partially unsaturated ring;
   each R^{c} is independently selected from halogen. —OR, —N(R)₂, —CN, and optionally substituted C₁₋₆ aliphatic; and
   m is 0, 1, 2, or 3.
445. The compound of Embodiment 444, wherein the compound is of Formula XA-1 or XA-2:

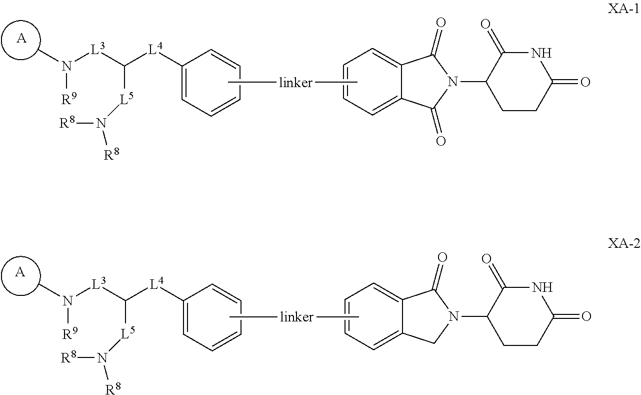

or a pharmaceutically acceptable salt thereof.
446. The compound of any one of claims 381-420, wherein LBM or

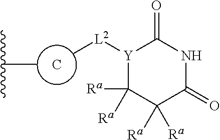

moiety is selected from:

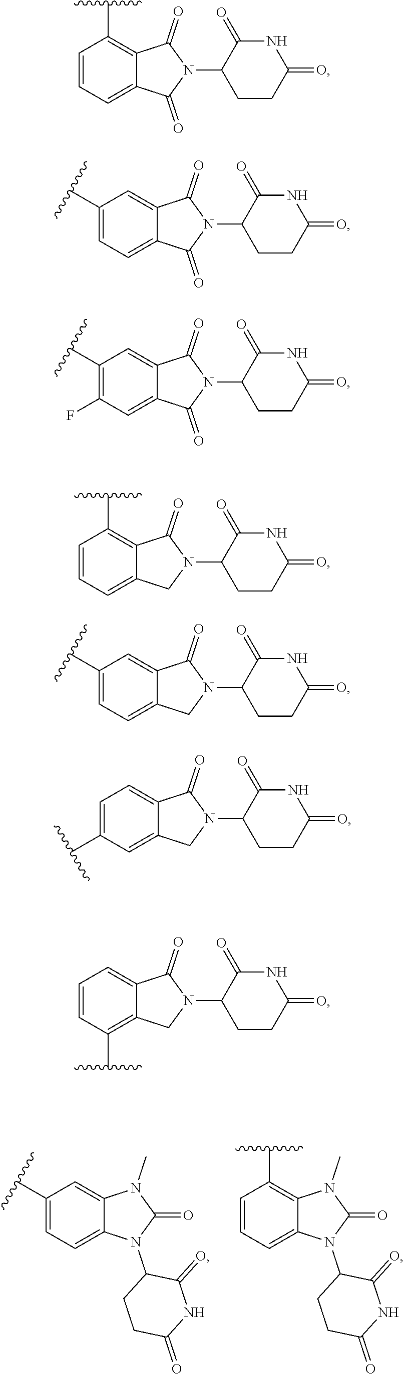

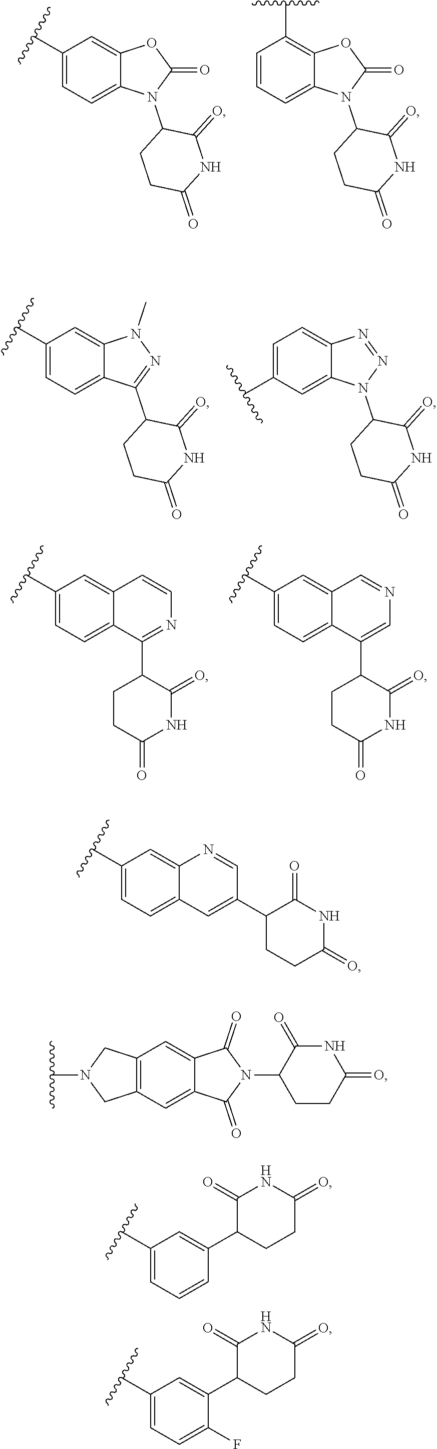

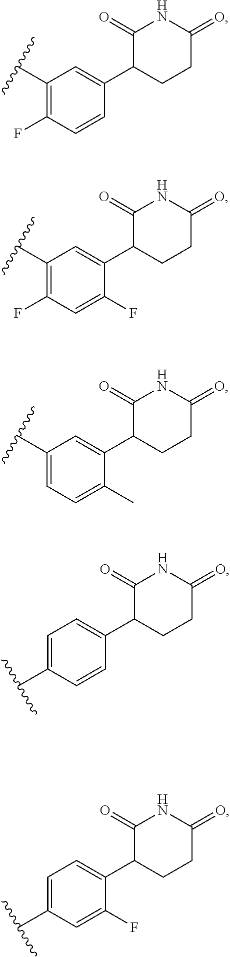 [SMILES: *c1cc(C2CCC(=O)NC2=O)ccc1F ; *c1cc(C2CCC(=O)NC2=O)c(F)cc1F ; *c1ccc(C)c(C2CCC(=O)NC2=O)c1 ; *c1ccc(C2CCC(=O)NC2=O)cc1 ; *c1ccc(C2CCC(=O)NC2=O)c(F)c1]

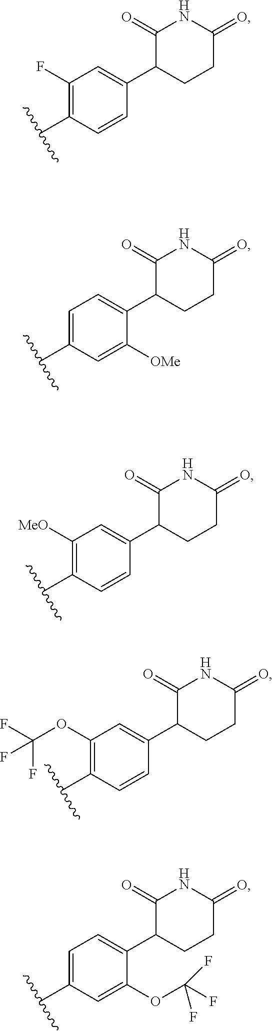 [SMILES: *c1ccc(C2CCC(=O)NC2=O)cc1F ; *c1ccc(C2CCC(=O)NC2=O)c(OC)c1 ; *c1ccc(C2CCC(=O)NC2=O)cc1OC ; *c1ccc(C2CCC(=O)NC2=O)cc1OC(F)(F)F ; *c1ccc(C2CCC(=O)NC2=O)c(OC(F)(F)F)c1]

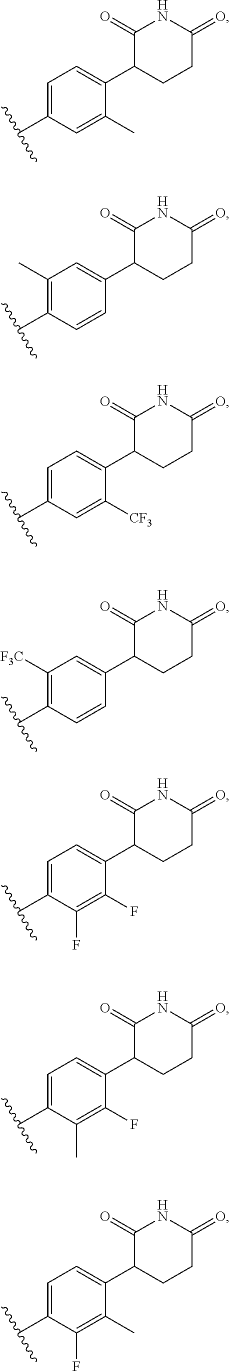

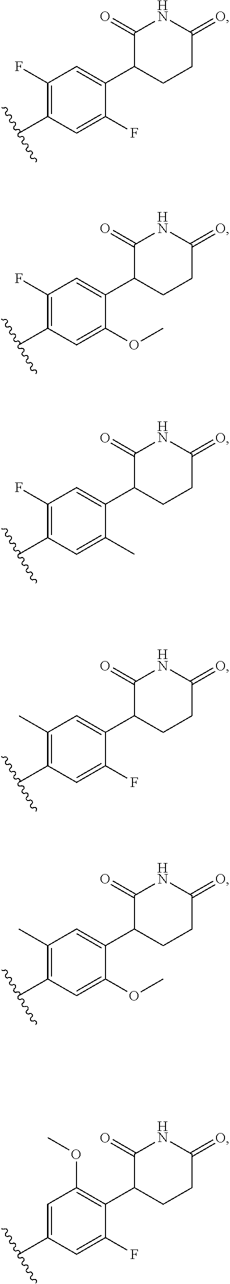 [SMILES: *c1cc(F)c(C2CCC(=O)NC2=O)cc1F ; *c1cc(OC)c(C2CCC(=O)NC2=O)cc1F ; *c1cc(C)c(C2CCC(=O)NC2=O)cc1F ; *c1cc(F)c(C2CCC(=O)NC2=O)cc1C ; *c1cc(OC)c(C2CCC(=O)NC2=O)cc1C ; *c1cc(F)c(C2CCC(=O)NC2=O)c(OC)c1]

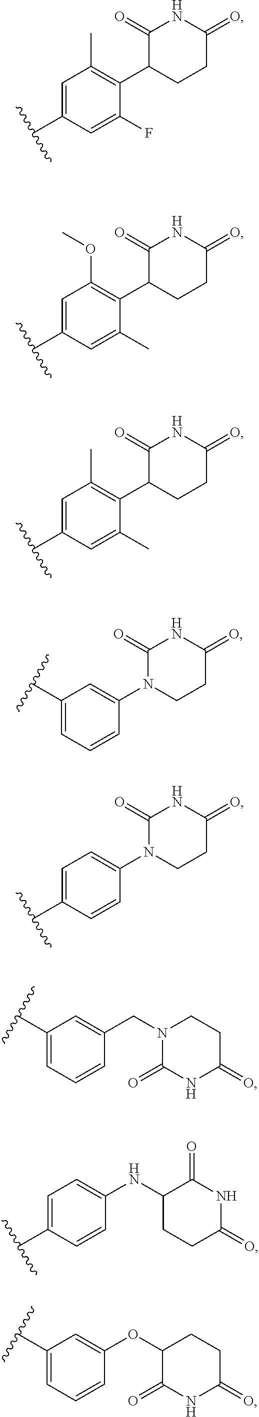

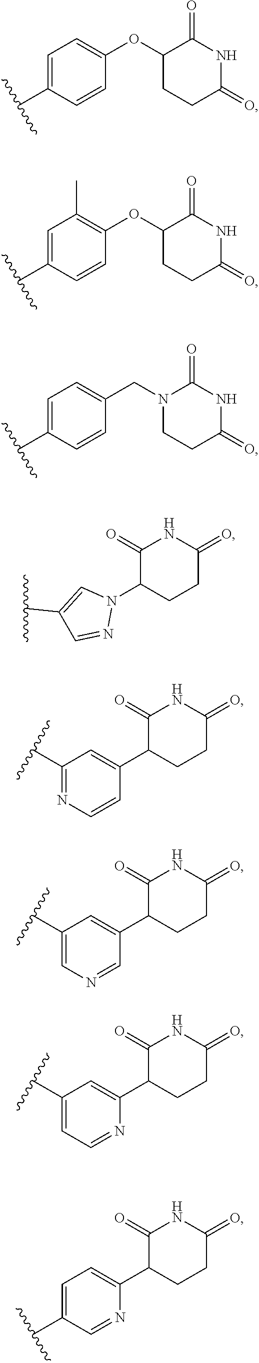

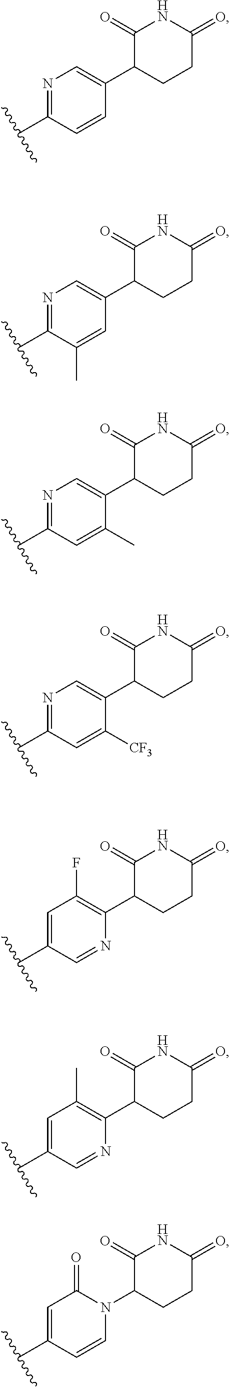

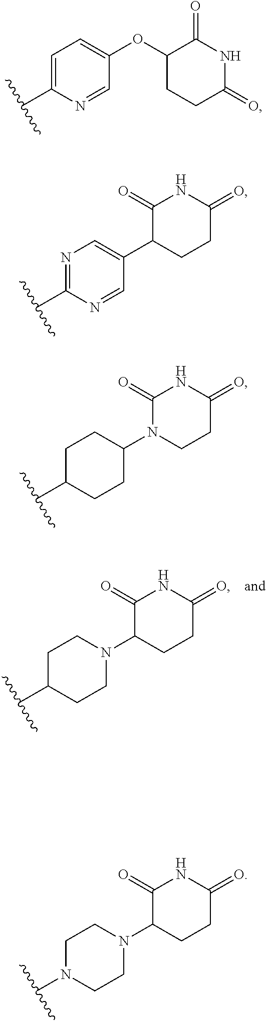 [SMILES: *c1ccc(OC2CCC(=O)NC2=O)cn1 ; *c1ncc(C2CCC(=O)NC2=O)cn1 ; *C1CCC(N2CCC(=O)NC2=O)CC1 ; *C1CCN(C2CCC(=O)NC2=O)CC1 ; "and" ; *N1CCN(C2CCC(=O)NC2=O)CC1]

447. The compound of any one of 381-420, wherein LBM or

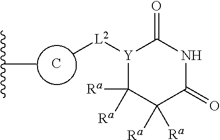

moiety is selected from:

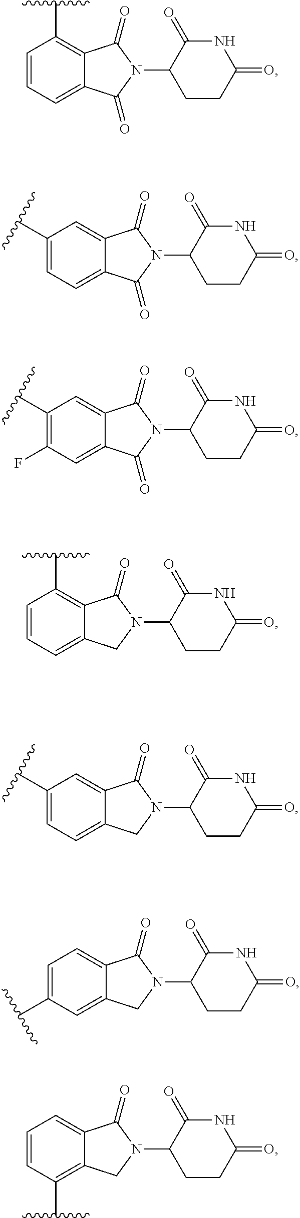

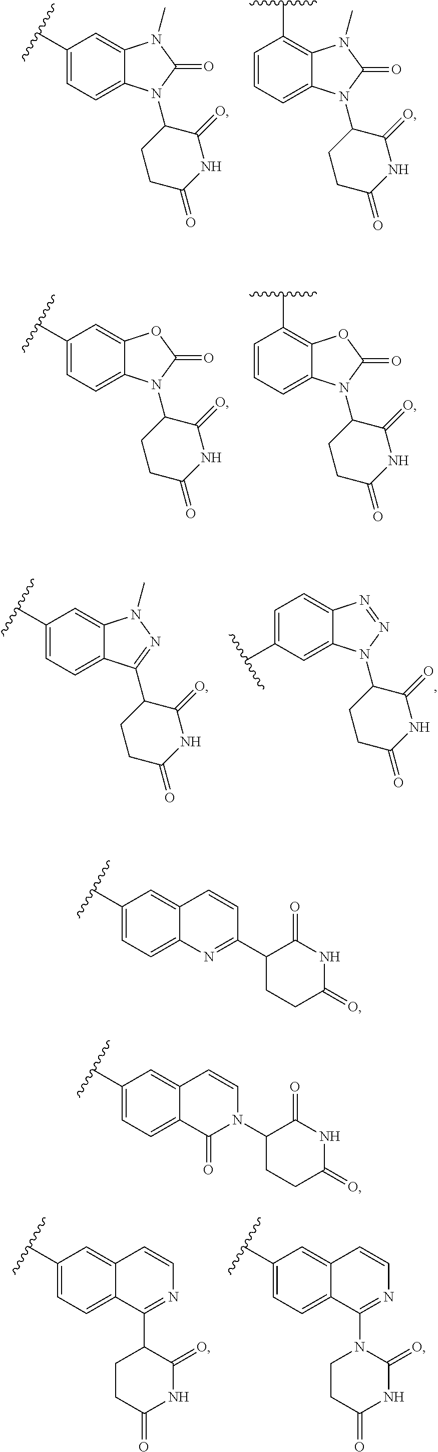

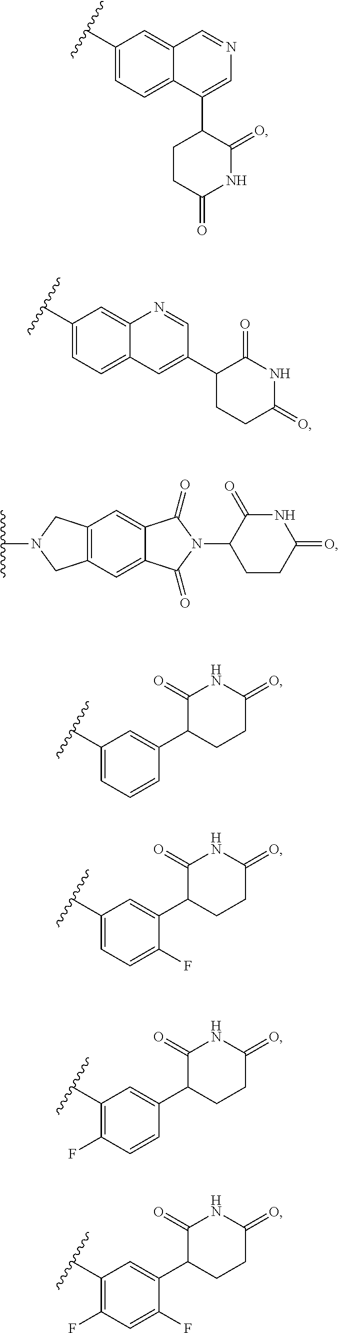

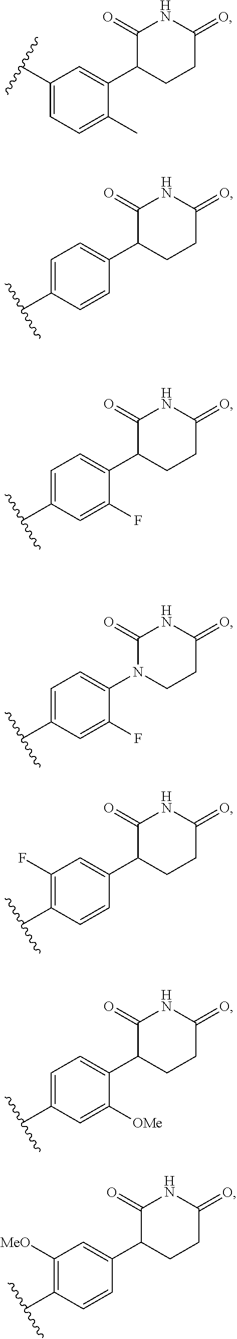

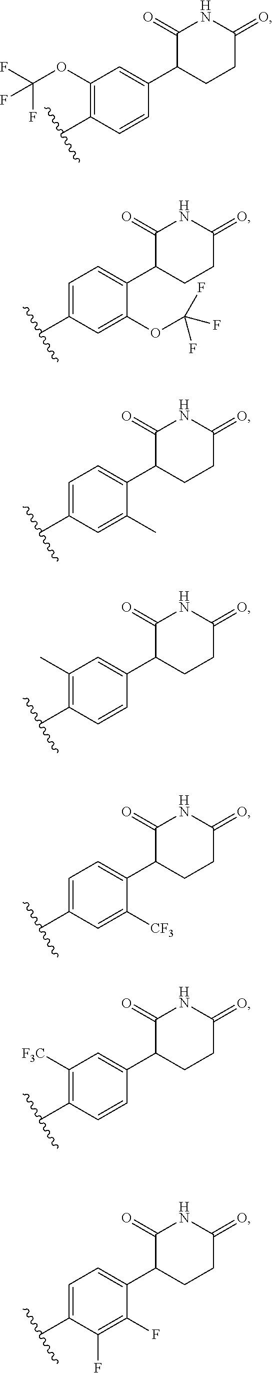

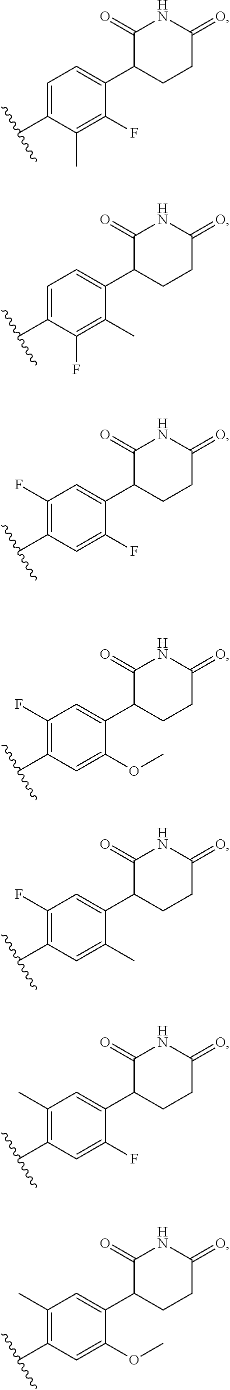

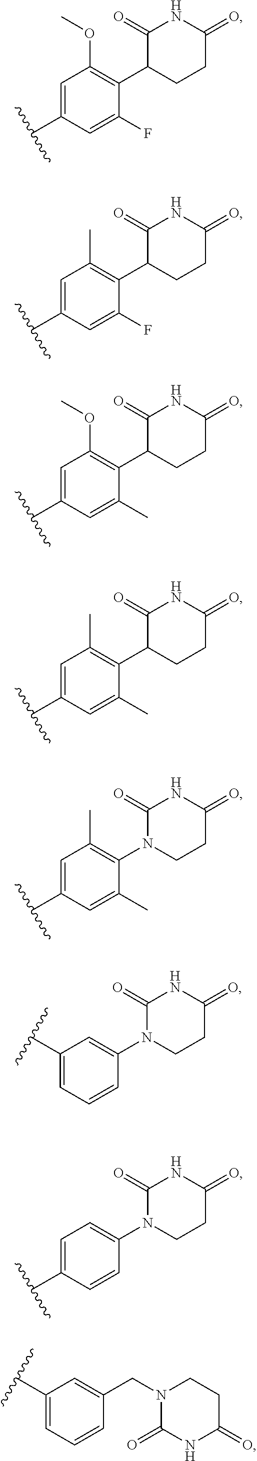

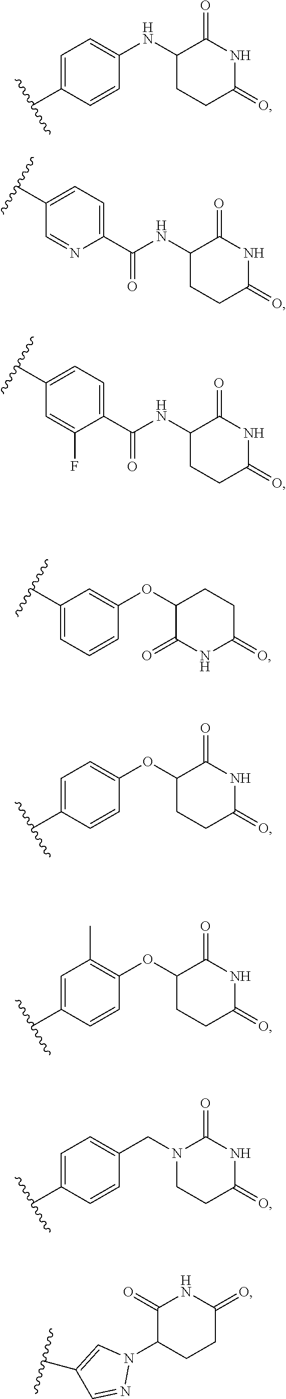

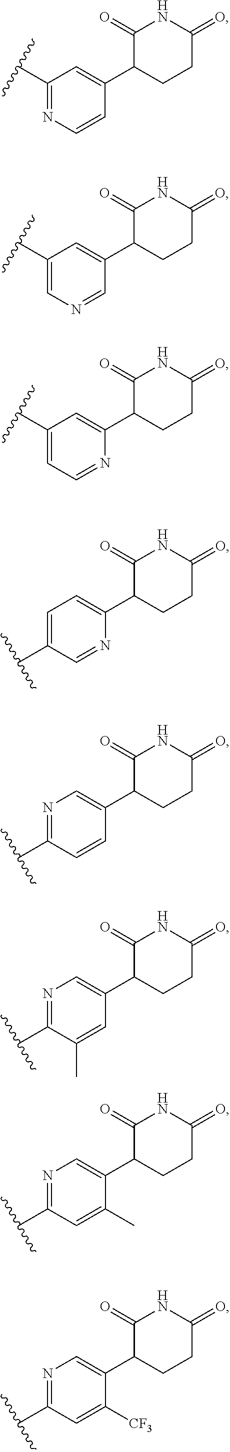

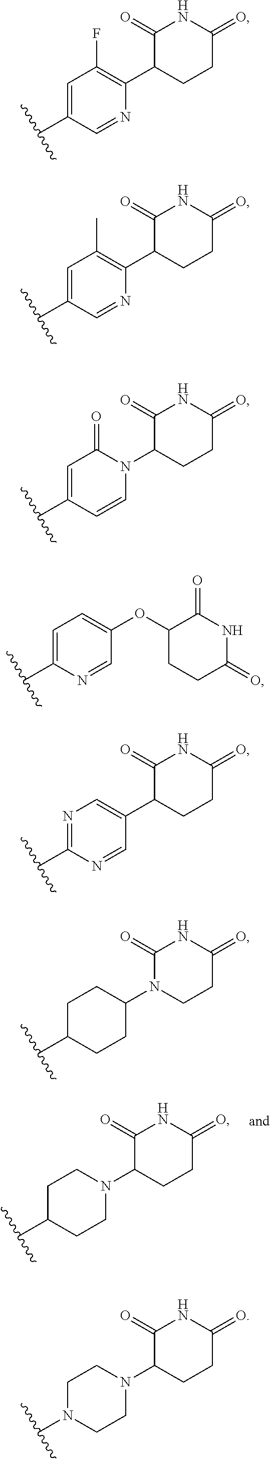

448. The compound of any one of Embodiments 246-380, wherein LBM is

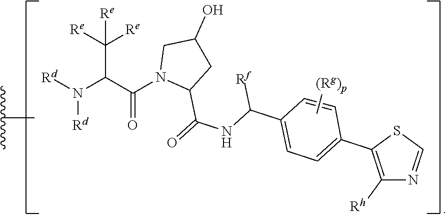

449. The compound of any one of Embodiments 246-380, wherein the compound is a compound of Formula XI-1:

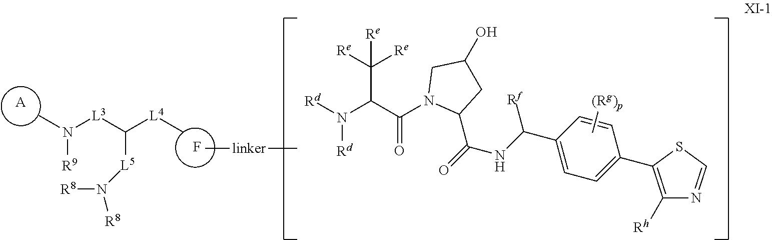

or a pharmaceutically acceptable salt thereof.
450. The compound of any one of Embodiments 246-380, wherein the compound is a compound of Formula XI:

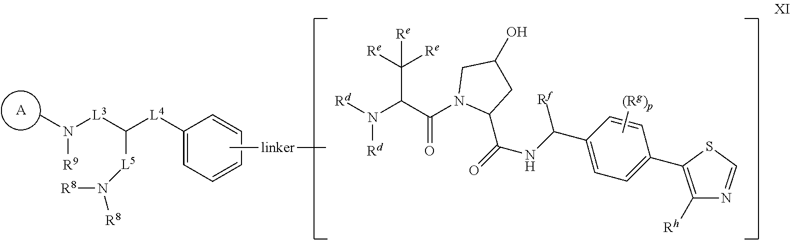

or a pharmaceutically acceptable salt thereof, wherein:
   linker is attached to the bracketed moiety at one of R^{d}, R^{e}, R^{f}, and R^{g};
   each R^{d} that is not the point of attachment for the linker is independently hydrogen, —C(O)R, or optionally substituted C₁₋₆ aliphatic, or
   two R^{d}, together with the atom to which they are attached, combine to form an optionally substituted 5- to 6-membered ring having 1-3 heteroatoms independently selected from N, O, and S and optionally fused to a phenyl or 5- to 6-membered heteroaryl ring having 1-3 heteroatoms independently selected from N, O, and S;
   each R^{e} that is not the point of attachment for the linker is independently hydrogen or optionally substituted C₁₋₆ aliphatic;
   R^{f}, when it is not the point of attachment for the linker, is hydrogen or optionally substituted C₁₋₆ aliphatic;
   each R^{g} that is not the point of attachment for the linker is independently halogen, —OR, —CN, or optionally substituted C₁₋₆ aliphatic;
   R^{h} is hydrogen, halogen, or optionally substituted C₁₋₆ aliphatic; and
   p is 0, 1, 2, or 3.
451. The compound of any one of Embodiments 448-450, wherein the compound is a compound of Formula XIA:

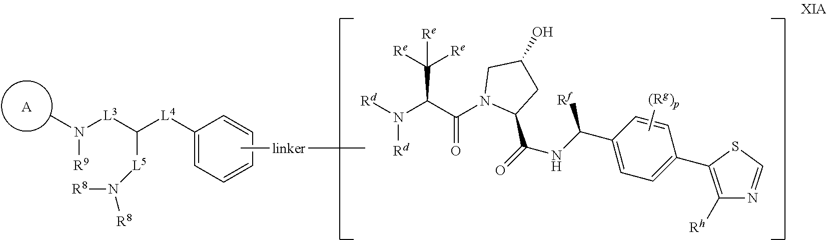

or a pharmaceutically acceptable salt thereof.
452. The compound of any one of Embodiments 246-380, wherein LBM is

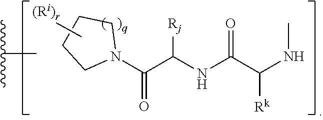

453. The compound of any one of Embodiments 246-380, wherein the compound is a compound of Formula XII-1:

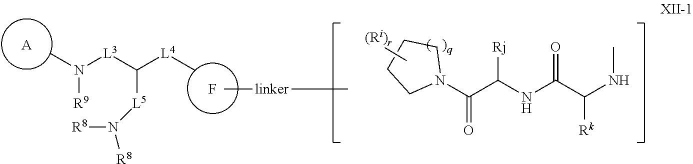

or a pharmaceutically acceptable salt thereof.
454. The compound of any one of Embodiments 246-380, wherein the compound is a compound of Formula XII:

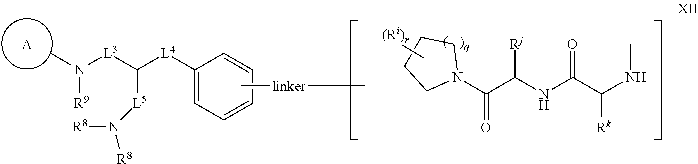

or a pharmaceutically acceptable salt thereof.
455. The compound of Embodiment 452-454, wherein at least one Rⁱ is —C(O)N(R)₂.
456. The compound of Embodiment 452-454, wherein two Rⁱ groups, together with the atoms to which they are attached, combine to form an optionally substituted phenyl or 5- to 6-membered heteroaryl ring having 1-3 heteroatoms independently selected from N, O, and S.
457. The compound of Embodiment 452-456, wherein the compound is a compound of Formula XIIA:

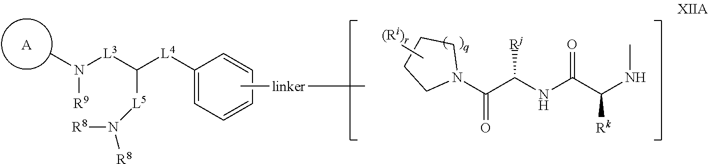

or a pharmaceutically acceptable salt thereof.
458. The compound of Embodiment 452-457, wherein the compound is a compound of Formula XIIB:

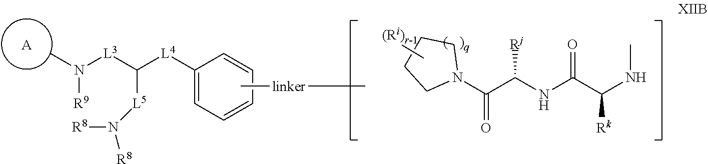

or a pharmaceutically acceptable salt thereof.
459. The compound of Embodiment 452-454, wherein the compound is a compound of Formula XIIB-1:

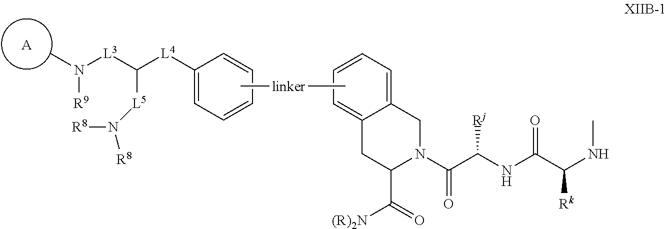

or a pharmaceutically acceptable salt thereof.
460. The compound of any one of Embodiments 246-380, wherein LBM is

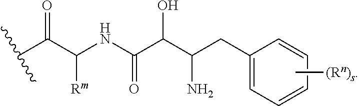

461. The compound of any one of Embodiments 246-380, wherein the compound is a compound of Formula XIII-1:

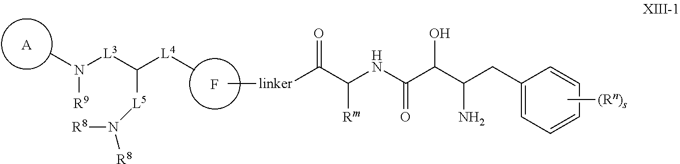

or a pharmaceutically acceptable salt thereof.
462. The compound of any one of Embodiments 246-380, wherein the compound is a compound of Formula XIII:

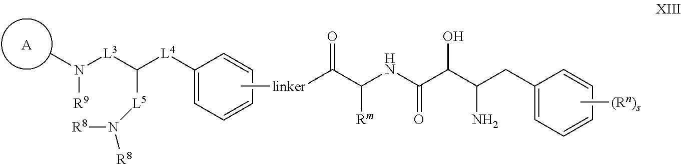

or a pharmaceutically acceptable salt thereof, wherein:
   R^{m} is optionally substituted C₁₋₆ aliphatic;
   each Rⁿ is independently halogen, —OR, —CN, or optionally substituted C₁₋₆ aliphatic; and
   s is 0, 1, 2, 3, 4, or 5.
463. The compound of any one of Embodiments 460-462, wherein the compound is a compound of Formula XIIIA:

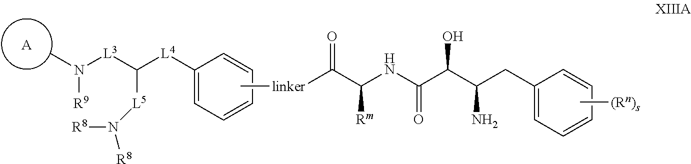

or a pharmaceutically acceptable salt thereof.
464. The compound of any one of Embodiments 246-380, wherein LBM is

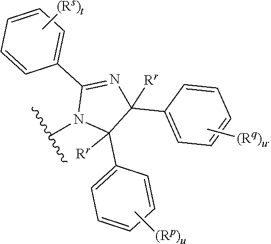

465. The compound of any one of Embodiments 246-380, wherein the compound is a compound of Formula XIV-1:

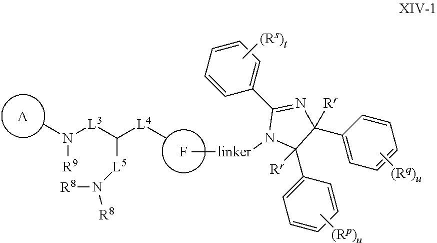

or a pharmaceutically acceptable salt thereof.
466. The compound of any one of Embodiments 246-380, wherein the compound is a compound of Formula XIV:

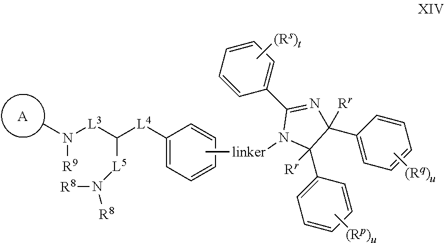

or a pharmaceutically acceptable salt thereof, wherein:
   each R^{p} is independently halogen, —OR, —CN, or optionally substituted C₁₋₆ aliphatic;
   each R^{q} is independently halogen, —OR, —CN, or optionally substituted C₁₋₆ aliphatic;
   each R^{r} is independently hydrogen or optionally substituted C₁₋₆ aliphatic;
   each R^{s} is independently halogen, —OR, —CN, or optionally substituted C₁₋₆ aliphatic;
   t is 0, 1, 2, 3, 4, or 5; and
   each u is independently 0, 1, 2, 3, 4, or 5.
467. The compound of any one of Embodiments 246-380, wherein LBM is

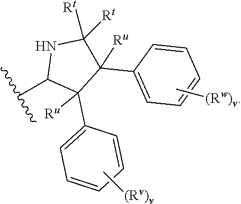

468. The compound of any one of Embodiments 246-380, wherein the compound is a compound of Formula XIV-1:

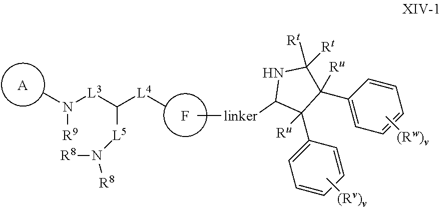

or a pharmaceutically acceptable salt thereof.
469. The compound of any one of Embodiments 246-380, wherein the compound is a compound of Formula XV:

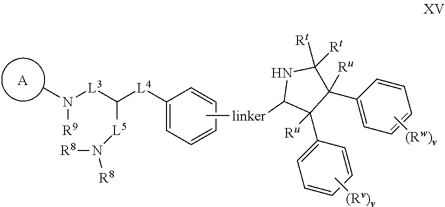

or a pharmaceutically acceptable salt thereof, wherein:
   each R^{t} is independently hydrogen or optionally substituted C₁₋₆ aliphatic, or
   both R^{t} groups, together with the atom to which they are attached, combine to form an optionally substituted 3- to 7-membered cycloaliphatic or heterocycle having 1-2 heteroatoms independently selected from N, O, and S;
   each R^{u} is independently hydrogen, halogen, —CN, or optionally substituted C₁₋₆ aliphatic;
   each R^{v} is independently halogen, —OR, —CN, or optionally substituted C₁₋₆ aliphatic, or
   one instance of R^{u} and R^{v}, together with the atoms to which they are attached combine to form an optionally substituted 3- to 7-membered cycloaliphatic or heterocycle having 1-2 heteroatoms independently selected from N, O, and S;
   each R^{w} is independently halogen, —OR, —CN, or optionally substituted C₁₋₆ aliphatic, or one instance of R^{u} and R^{w}, together with the atoms to which they are attached combine to form an optionally substituted 3- to 7-membered cycloaliphatic or heterocycle having 1-2 heteroatoms independently selected from N, O, and S; and
   each v is independently 0, 1, 2, 3, 4, or 5.
470. A compound selected from Table 1 or Table 2, or a pharmaceutically acceptable salt thereof.
471. A compound, wherein the compound is:

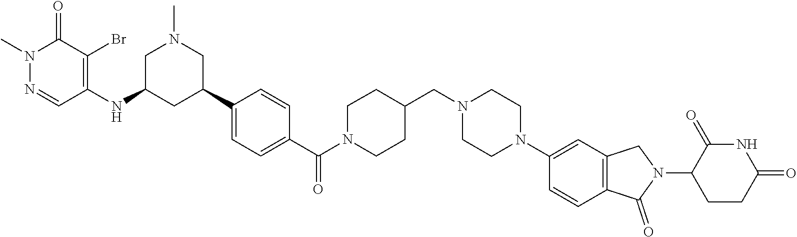 [SMILES: CN1C[C@H](Nc2cnn(C)c(=O)c2Br)C[C@H](c2ccc(C(=O)N3CCC(CN4CCN(c5ccc6c(c5)CN(C5CCC(=O)NC5=O)C6=O)CC4)CC3)cc2)C1]

or a pharmaceutically acceptable salt thereof.
472. A compound, wherein the compound is:

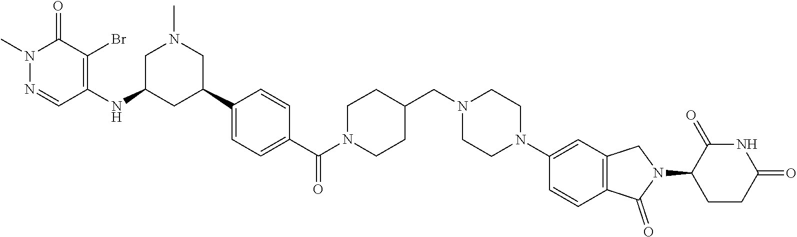 [SMILES: CN1C[C@H](Nc2cnn(C)c(=O)c2Br)C[C@H](c2ccc(C(=O)N3CCC(CN4CCN(c5ccc6c(c5)CN([C@@H]5CCC(=O)NC5=O)C6=O)CC4)CC3)cc2)C1]

or a pharmaceutically acceptable salt thereof.
473. A compound, wherein the compound is:

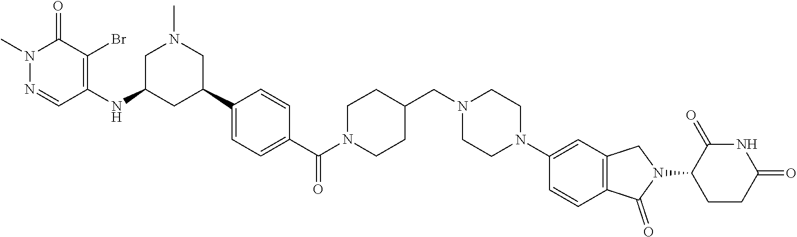 [SMILES: CN1C[C@H](Nc2cnn(C)c(=O)c2Br)C[C@H](c2ccc(C(=O)N3CCC(CN4CCN(c5ccc6c(c5)CN([C@H]5CCC(=O)NC5=O)C6=O)CC4)CC3)cc2)C1]

or a pharmaceutically acceptable salt thereof.
474. A compound, wherein the compound is:

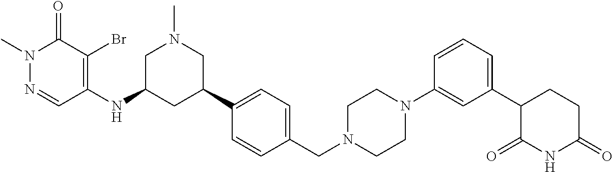 [SMILES: CN1C[C@H](Nc2cnn(C)c(=O)c2Br)C[C@H](c2ccc(CN3CCN(c4cccc(C5CCC(=O)NC5=O)c4)CC3)cc2)C1]

or a pharmaceutically acceptable salt thereof.
475. A compound, wherein the compound is:

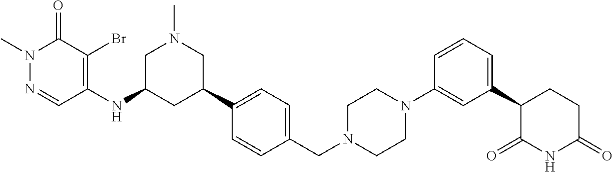 [SMILES: CN1C[C@H](Nc2cnn(C)c(=O)c2Br)C[C@H](c2ccc(CN3CCN(c4cccc([C@H]5CCC(=O)NC5=O)c4)CC3)cc2)C1]

or a pharmaceutically acceptable salt thereof.
476. A compound, wherein the compound is:

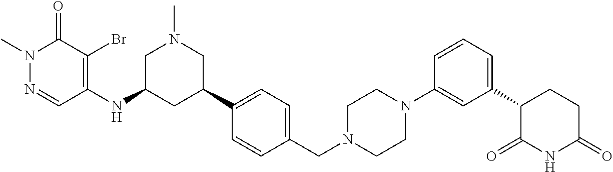 [SMILES: CN1C[C@H](Nc2cnn(C)c(=O)c2Br)C[C@H](c2ccc(CN3CCN(c4cccc([C@@H]5CCC(=O)NC5=O)c4)CC3)cc2)C1]

or a pharmaceutically acceptable salt thereof.
477. A compound, wherein the compound is:

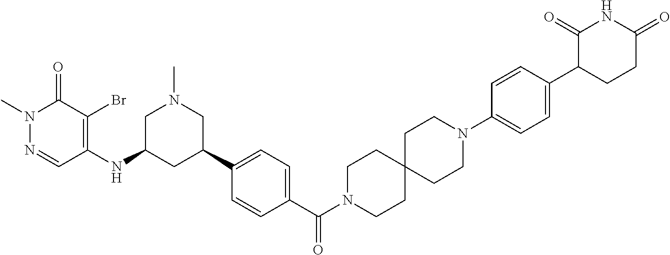 [SMILES: CN1C[C@H](Nc2cnn(C)c(=O)c2Br)C[C@H](c2ccc(C(=O)N3CCC4(CC3)CCN(c3ccc(C5CCC(=O)NC5=O)cc3)CC4)cc2)C1]

or a pharmaceutically acceptable salt thereof.
478. A compound, wherein the compound is:

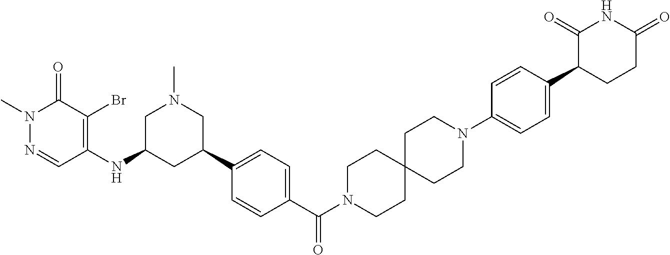 [SMILES: CN1C[C@H](Nc2cnn(C)c(=O)c2Br)C[C@H](c2ccc(C(=O)N3CCC4(CC3)CCN(c3ccc([C@@H]5CCC(=O)NC5=O)cc3)CC4)cc2)C1]

or a pharmaceutically acceptable salt thereof.
479. A compound, wherein the compound is:

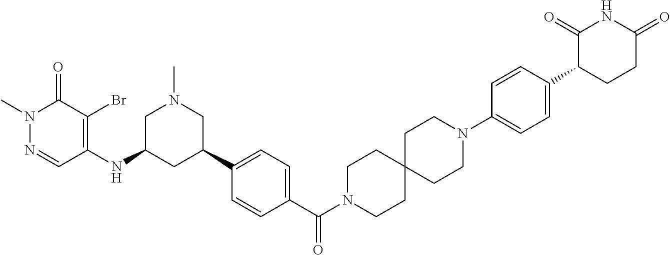 [SMILES: CN1C[C@H](Nc2cnn(C)c(=O)c2Br)C[C@H](c2ccc(C(=O)N3CCC4(CC3)CCN(c3ccc([C@H]5CCC(=O)NC5=O)cc3)CC4)cc2)C1]

or a pharmaceutically acceptable salt thereof.
480. A compound, wherein the compound is:

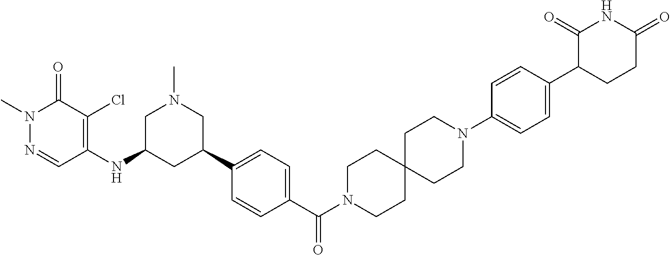 [SMILES: CN1C[C@H](Nc2cnn(C)c(=O)c2Cl)C[C@H](c2ccc(C(=O)N3CCC4(CC3)CCN(c3ccc(C5CCC(=O)NC5=O)cc3)CC4)cc2)C1]

or a pharmaceutically acceptable salt thereof.
481. A compound, wherein the compound is:

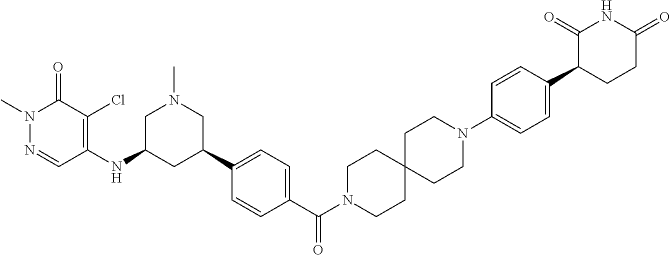 [SMILES: CN1C[C@H](Nc2cnn(C)c(=O)c2Cl)C[C@H](c2ccc(C(=O)N3CCC4(CC3)CCN(c3ccc([C@@H]5CCC(=O)NC5=O)cc3)CC4)cc2)C1]

or a pharmaceutically acceptable salt thereof.
482. A compound, wherein the compound is:

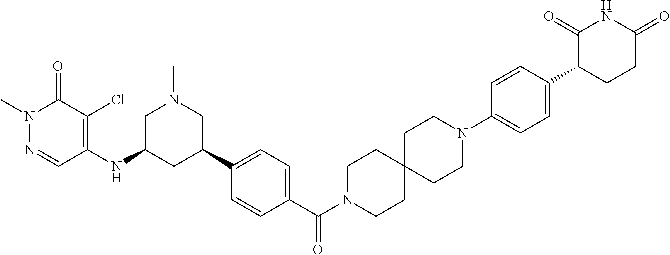 [SMILES: CN1C[C@H](Nc2cnn(C)c(=O)c2Cl)C[C@H](c2ccc(C(=O)N3CCC4(CC3)CCN(c3ccc([C@H]5CCC(=O)NC5=O)cc3)CC4)cc2)C1]

or a pharmaceutically acceptable salt thereof.
483. A compound, wherein the compound is:

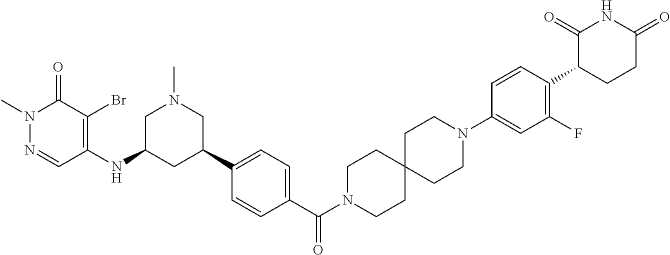 [SMILES: CN1C[C@H](Nc2cnn(C)c(=O)c2Br)C[C@H](c2ccc(C(=O)N3CCC4(CC3)CCN(c3ccc([C@H]5CCC(=O)NC5=O)c(F)c3)CC4)cc2)C1]

or a pharmaceutically acceptable salt thereof.
484. A compound, wherein the compound is:

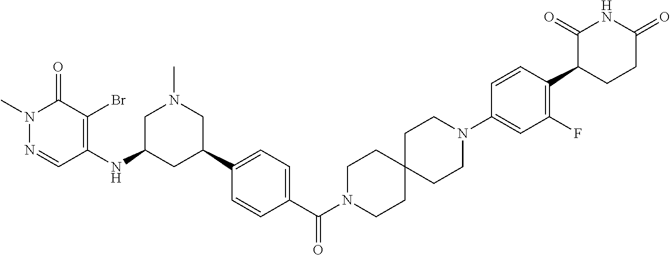 [SMILES: CN1C[C@H](Nc2cnn(C)c(=O)c2Br)C[C@H](c2ccc(C(=O)N3CCC4(CC3)CCN(c3ccc([C@@H]5CCC(=O)NC5=O)c(F)c3)CC4)cc2)C1]

or a pharmaceutically acceptable salt thereof.
485. A compound, wherein the compound is:

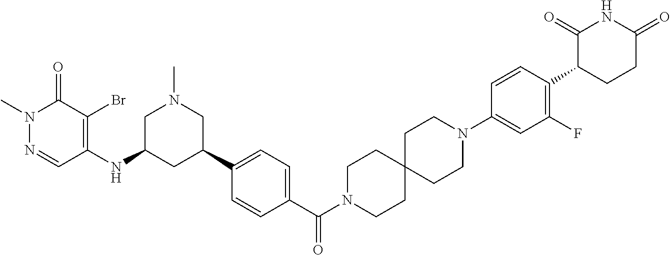 [SMILES: CN1C[C@H](Nc2cnn(C)c(=O)c2Br)C[C@H](c2ccc(C(=O)N3CCC4(CC3)CCN(c3ccc([C@H]5CCC(=O)NC5=O)c(F)c3)CC4)cc2)C1]

or a pharmaceutically acceptable salt thereof.
486. A compound, wherein the compound is:

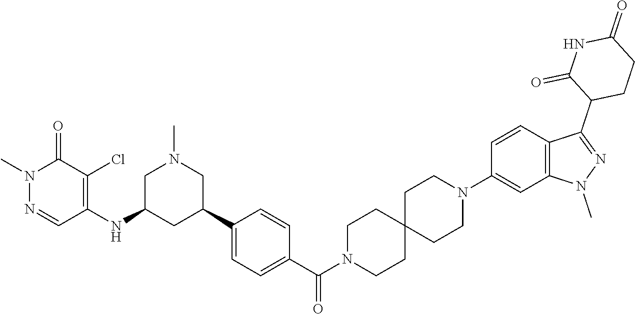 [SMILES: CN1C[C@H](Nc2cnn(C)c(=O)c2Cl)C[C@H](c2ccc(C(=O)N3CCC4(CC3)CCN(c3ccc5c(C6CCC(=O)NC6=O)nn(C)c5c3)CC4)cc2)C1]

or a pharmaceutically acceptable salt thereof.
487. A compound, wherein the compound is:

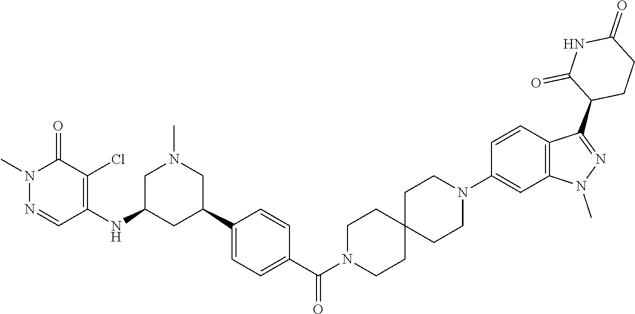 [SMILES: CN1C[C@H](Nc2cnn(C)c(=O)c2Cl)C[C@H](c2ccc(C(=O)N3CCC4(CC3)CCN(c3ccc5c([C@@H]6CCC(=O)NC6=O)nn(C)c5c3)CC4)cc2)C1]

or a pharmaceutically acceptable salt thereof.
488. A compound, wherein the compound is:

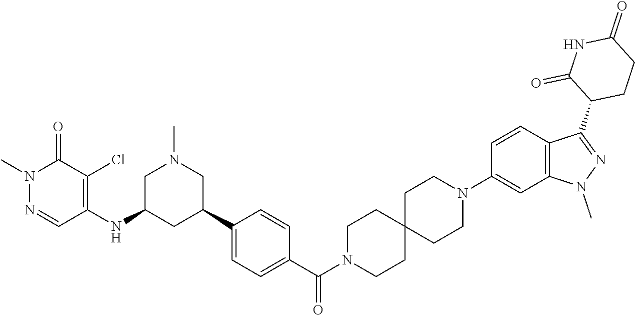 [SMILES: CN1C[C@H](Nc2cnn(C)c(=O)c2Cl)C[C@H](c2ccc(C(=O)N3CCC4(CC3)CCN(c3ccc5c([C@H]6CCC(=O)NC6=O)nn(C)c5c3)CC4)cc2)C1]

or a pharmaceutically acceptable salt thereof.
489. A compound, wherein the compound is:

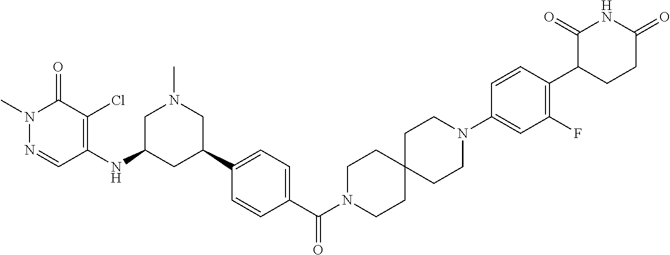 [SMILES: CN1C[C@H](Nc2cnn(C)c(=O)c2Cl)C[C@H](c2ccc(C(=O)N3CCC4(CC3)CCN(c3ccc(C5CCC(=O)NC5=O)c(F)c3)CC4)cc2)C1]

or a pharmaceutically acceptable salt thereof.
490. A compound, wherein the compound is:

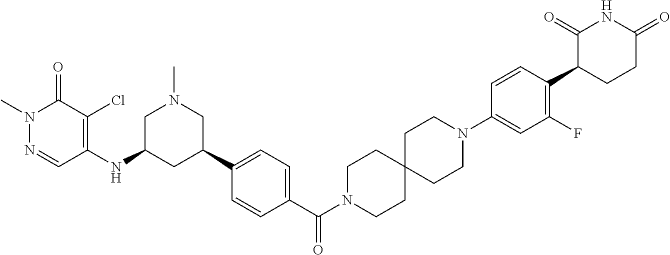 [SMILES: CN1C[C@H](Nc2cnn(C)c(=O)c2Cl)C[C@H](c2ccc(C(=O)N3CCC4(CC3)CCN(c3ccc([C@@H]5CCC(=O)NC5=O)c(F)c3)CC4)cc2)C1]

or a pharmaceutically acceptable salt thereof.
491. A compound, wherein the compound is:

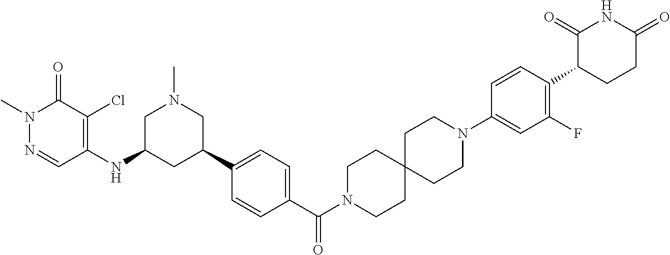 [SMILES: CN1C[C@H](Nc2cnn(C)c(=O)c2Cl)C[C@H](c2ccc(C(=O)N3CCC4(CC3)CCN(c3ccc([C@H]5CCC(=O)NC5=O)c(F)c3)CC4)cc2)C1]

or a pharmaceutically acceptable salt thereof.
492. A compound, wherein the compound is:

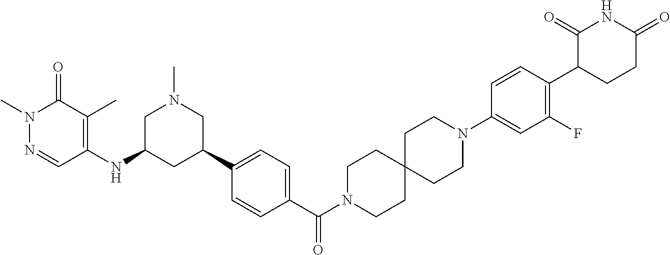 [SMILES: Cc1c(N[C@@H]2C[C@H](c3ccc(C(=O)N4CCC5(CC4)CCN(c4ccc(C6CCC(=O)NC6=O)c(F)c4)CC5)cc3)CN(C)C2)cnn(C)c1=O]

or a pharmaceutically acceptable salt thereof.
493. A compound, wherein the compound is:

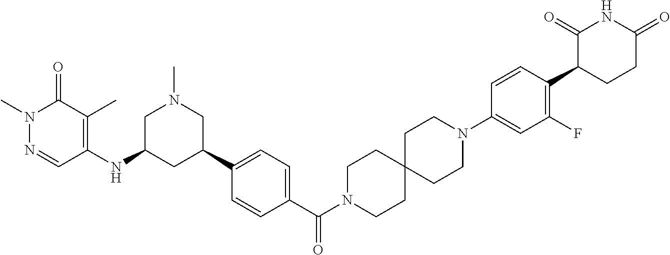 [SMILES: Cc1c(N[C@@H]2C[C@H](c3ccc(C(=O)N4CCC5(CC4)CCN(c4ccc([C@@H]6CCC(=O)NC6=O)c(F)c4)CC5)cc3)CN(C)C2)cnn(C)c1=O]

or a pharmaceutically acceptable salt thereof.
494. A compound, wherein the compound is:

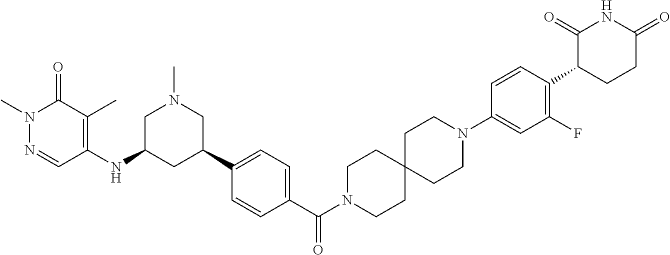 [SMILES: Cc1c(N[C@@H]2C[C@H](c3ccc(C(=O)N4CCC5(CC4)CCN(c4ccc([C@H]6CCC(=O)NC6=O)c(F)c4)CC5)cc3)CN(C)C2)cnn(C)c1=O]

or a pharmaceutically acceptable salt thereof.
495. A compound, wherein the compound is:

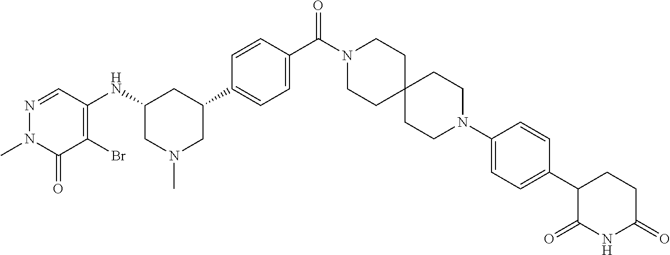 [SMILES: CN1C[C@H](Nc2cnn(C)c(=O)c2Br)C[C@H](c2ccc(C(=O)N3CCC4(CC3)CCN(c3ccc(C5CCC(=O)NC5=O)cc3)CC4)cc2)C1]

or a pharmaceutically acceptable salt thereof.
496. A compound, wherein the compound is:

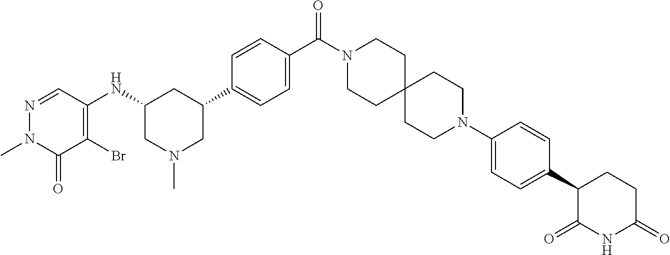 [SMILES: CN1C[C@H](Nc2cnn(C)c(=O)c2Br)C[C@H](c2ccc(C(=O)N3CCC4(CC3)CCN(c3ccc([C@H]5CCC(=O)NC5=O)cc3)CC4)cc2)C1]

or a pharmaceutically acceptable salt thereof.
497. A compound, wherein the compound is:

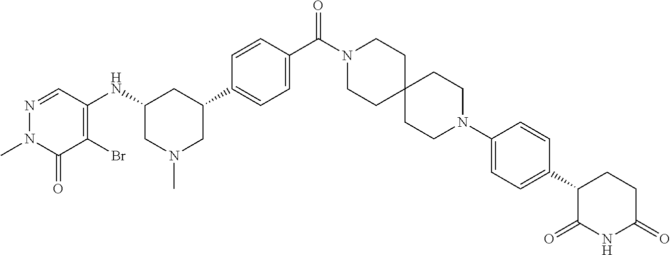 [SMILES: CN1C[C@H](Nc2cnn(C)c(=O)c2Br)C[C@H](c2ccc(C(=O)N3CCC4(CC3)CCN(c3ccc([C@@H]5CCC(=O)NC5=O)cc3)CC4)cc2)C1]

or a pharmaceutically acceptable salt thereof.
498. A compound, wherein the compound is:

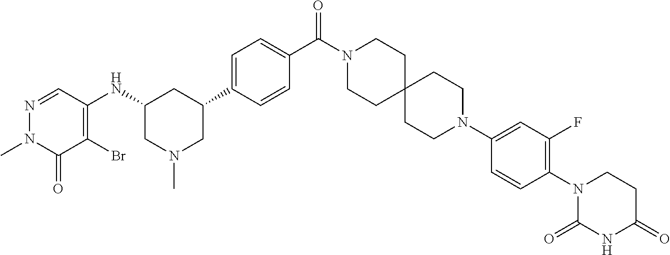 [SMILES: CN1C[C@H](Nc2cnn(C)c(=O)c2Br)C[C@H](c2ccc(C(=O)N3CCC4(CC3)CCN(c3ccc(N5CCC(=O)NC5=O)c(F)c3)CC4)cc2)C1]

or a pharmaceutically acceptable salt thereof.
499. A compound, wherein the compound is:

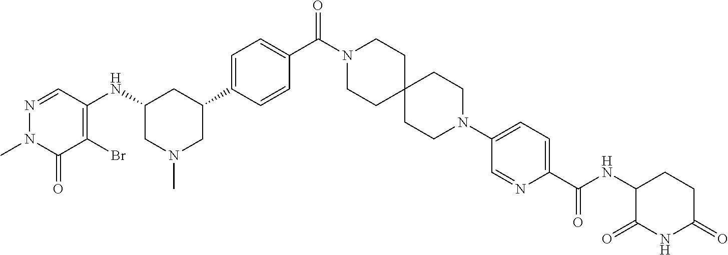 [SMILES: CN1C[C@H](Nc2cnn(C)c(=O)c2Br)C[C@H](c2ccc(C(=O)N3CCC4(CC3)CCN(c3ccc(C(=O)NC5CCC(=O)NC5=O)nc3)CC4)cc2)C1]

or a pharmaceutically acceptable salt thereof.
500. A compound, wherein the compound is:

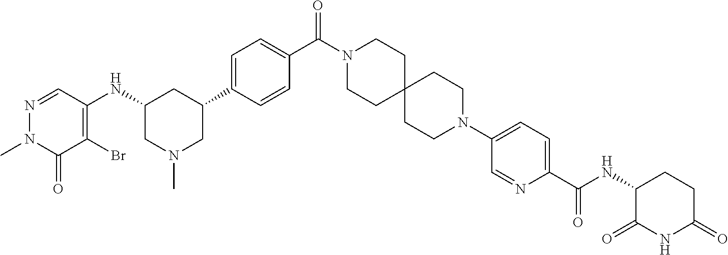 [SMILES: CN1C[C@H](Nc2cnn(C)c(=O)c2Br)C[C@H](c2ccc(C(=O)N3CCC4(CC3)CCN(c3ccc(C(=O)N[C@@H]5CCC(=O)NC5=O)nc3)CC4)cc2)C1]

or a pharmaceutically acceptable salt thereof.
501. A compound, wherein the compound is:

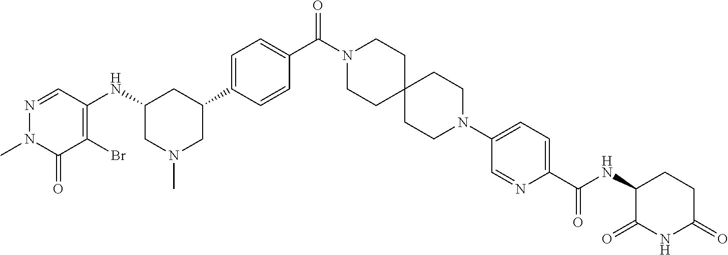 [SMILES: CN1C[C@H](Nc2cnn(C)c(=O)c2Br)C[C@H](c2ccc(C(=O)N3CCC4(CC3)CCN(c3ccc(C(=O)N[C@H]5CCC(=O)NC5=O)nc3)CC4)cc2)C1]

or a pharmaceutically acceptable salt thereof.
502. A compound, wherein the compound is:

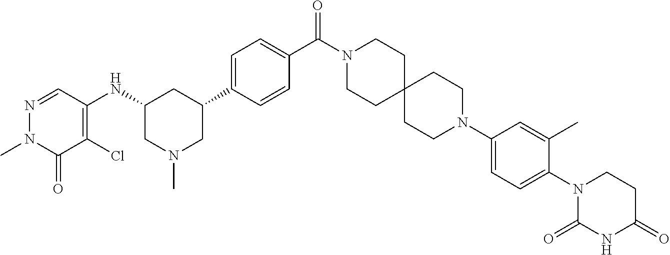 [SMILES: Cc1cc(N2CCC3(CCN(C(=O)c4ccc([C@H]5C[C@@H](Nc6cnn(C)c(=O)c6Cl)CN(C)C5)cc4)CC3)CC2)ccc1N1CCC(=O)NC1=O]

or a pharmaceutically acceptable salt thereof.
503. A compound, wherein the compound is:

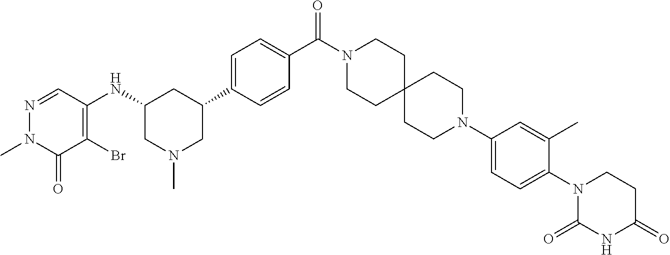 [SMILES: Cc1cc(N2CCC3(CCN(C(=O)c4ccc([C@H]5C[C@@H](Nc6cnn(C)c(=O)c6Br)CN(C)C5)cc4)CC3)CC2)ccc1N1CCC(=O)NC1=O]

or a pharmaceutically acceptable salt thereof.
504. A compound, wherein the compound is:

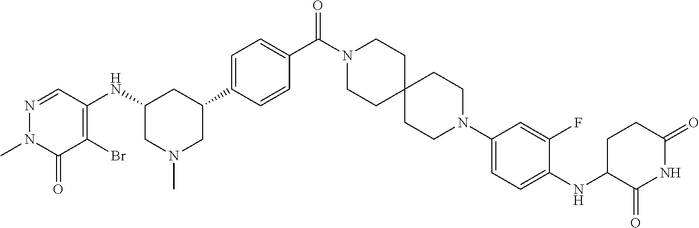 [SMILES: CN1C[C@H](Nc2cnn(C)c(=O)c2Br)C[C@H](c2ccc(C(=O)N3CCC4(CC3)CCN(c3ccc(NC5CCC(=O)NC5=O)c(F)c3)CC4)cc2)C1]

or a pharmaceutically acceptable salt thereof.
505. A compound, wherein the compound is:

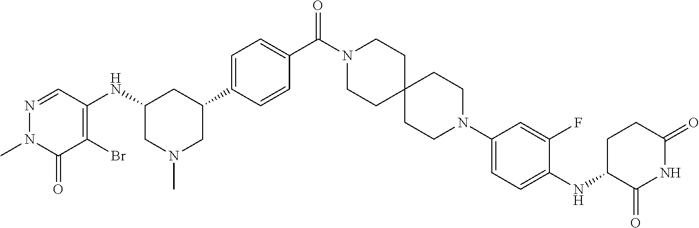 [SMILES: CN1C[C@H](Nc2cnn(C)c(=O)c2Br)C[C@H](c2ccc(C(=O)N3CCC4(CC3)CCN(c3ccc(N[C@@H]5CCC(=O)NC5=O)c(F)c3)CC4)cc2)C1]

or a pharmaceutically acceptable salt thereof.
506. A compound, wherein the compound is:

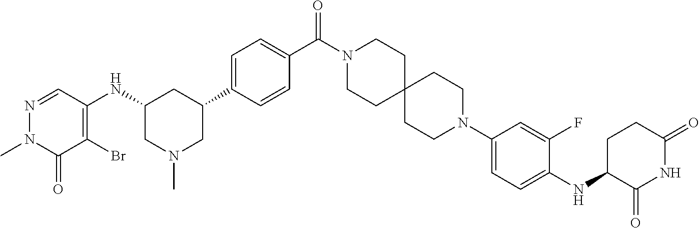 [SMILES: CN1C[C@H](Nc2cnn(C)c(=O)c2Br)C[C@H](c2ccc(C(=O)N3CCC4(CC3)CCN(c3ccc(N[C@H]5CCC(=O)NC5=O)c(F)c3)CC4)cc2)C1]

or a pharmaceutically acceptable salt thereof.
507. A compound, wherein the compound is:

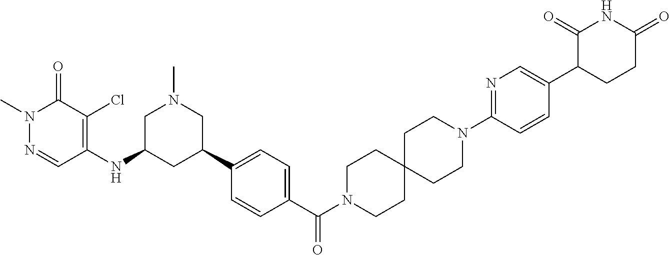 [SMILES: CN1C[C@H](Nc2cnn(C)c(=O)c2Cl)C[C@H](c2ccc(C(=O)N3CCC4(CC3)CCN(c3ccc(C5CCC(=O)NC5=O)cn3)CC4)cc2)C1]

or a pharmaceutically acceptable salt thereof.
508. A compound, wherein the compound is:

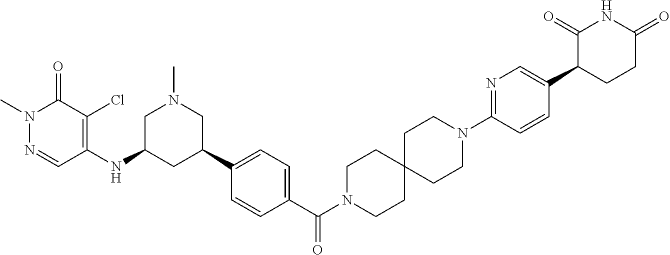 [SMILES: CN1C[C@H](Nc2cnn(C)c(=O)c2Cl)C[C@H](c2ccc(C(=O)N3CCC4(CC3)CCN(c3ccc([C@@H]5CCC(=O)NC5=O)cn3)CC4)cc2)C1]

or a pharmaceutically acceptable salt thereof.
509. A compound, wherein the compound is:

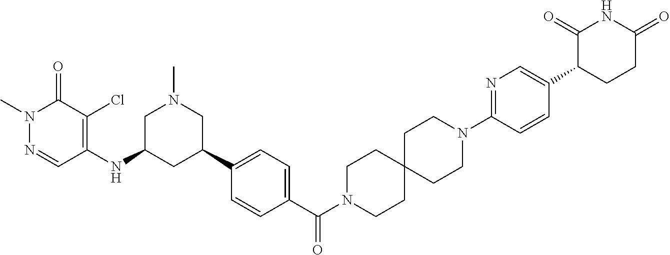 [SMILES: CN1C[C@H](Nc2cnn(C)c(=O)c2Cl)C[C@H](c2ccc(C(=O)N3CCC4(CC3)CCN(c3ccc([C@H]5CCC(=O)NC5=O)cn3)CC4)cc2)C1]

or a pharmaceutically acceptable salt thereof.
510. The compound of any one of the preceding Embodiments, wherein the compound is in a pharmaceutically acceptable salt form.
511. The compound of any one of the preceding Embodiments, wherein the stereochemical purity of each chiral center in PBM is at least about 90%.
512. The compound of any one of the preceding Embodiments, wherein the stereochemical purity of each chiral center is at least about 90%.
513. The compound of any one of the preceding Embodiments, wherein purity of the compound by qNMR is at least about 90% (wt %).
514. A pharmaceutical composition comprising a compound of any one of any one of the preceding Embodiments, or a pharmaceutically acceptable salt thereof, and a pharmaceutically acceptable carrier.
515. The composition of Embodiment 514, wherein the composition further comprises one or more stereoisomers of the compound, wherein the percentage of the compound, among the total of the compound and all its stereoisomers in the composition, is about or at least about 90%.
516. The composition of Embodiment 514, wherein the composition further comprises one or more stereoisomers of the compound, wherein the percentage of the compound, among the total of the compound and all its stereoisomers in the composition, is about or at least about 20%-80%.
517. The composition of any one of Embodiments 514-516, wherein the salt is a citrate salt.
518. The composition of any one of Embodiments 514-516, wherein the composition comprises a buffer or components thereof.
519. The composition of Embodiment 518, wherein the buffer is a citrate buffer.
520. The composition of any one of Embodiments 518-519, wherein the pH of the buffer is about 2-6.5.
521. The composition of any one of Embodiments 514-520, wherein the composition comprises a cyclodextrin.
522. The composition of Embodiment 521, wherein the composition comprises HPBCD.
523. The composition of any one of Embodiments 514-520, wherein the composition is substantially free of cyclodextrin.
524. A method of preparing a pharmaceutical composition of any one of Embodiments 514-523, comprising:
   providing the compound of any one of Embodiments 1-514, or a pharmaceutically acceptable salt thereof; and
   formulating the compound with suitable excipients to give the pharmaceutical composition.
525. A method for reducing KAT2A in a system, comprising administering or delivering to the system an effective amount of a compound of any one of the preceding Embodiments, or a pharmaceutically acceptable salt thereof, or a composition of any one of the preceding Embodiments.
526. A method for reducing KAT2B in a system, comprising administering or delivering to the system an effective amount of a compound of any one of the preceding Embodiments, or a pharmaceutically acceptable salt thereof, or a composition of any one of the preceding Embodiments.
527. A method for reducing KAT2A and KAT2B in a system, comprising administering or delivering to the system an effective amount of a compound of any one of the preceding Embodiments, or a pharmaceutically acceptable salt thereof, or a composition of any one of the preceding Embodiments.
528. A method for reducing histone acetylation in a system, comprising administering or delivering to the system an effective amount of a compound of any one of the preceding Embodiments, or a pharmaceutically acceptable salt thereof, or a composition of any one of the preceding Embodiments.
529. The method of Embodiment 528, wherein H3K9 acetylation is reduced
530. A method for inducing or promoting cell differentiation in a system, comprising administering or delivering to the system an effective amount of a compound of any one of the preceding Embodiments, or a pharmaceutically acceptable salt thereof, or a composition of any one of the preceding Embodiments.
531. A method for reducing or blocking de-differentiation in a system, comprising administering or delivering to the system an effective amount of a compound of any one of the preceding Embodiments, or a pharmaceutically acceptable salt thereof, or a composition of any one of the preceding Embodiments.
532. A method for reducing or blocking trans-differentiation in a system, comprising administering or delivering to the system an effective amount of a compound of any one of the preceding Embodiments, or a pharmaceutically acceptable salt thereof, or a composition of any one of the preceding Embodiments.
533. The method of any one of Embodiments 528-532, wherein the system comprises or expresses KAT2A and/or KAT2B.
534. The method of any one of Embodiments 525-533, wherein a system is or comprises a cell.
535. The method of any one of Embodiments 525-533, wherein a system is or comprises a cancer cell.
536. The method of any one of Embodiments 525-533, wherein a system is or comprises an acute myeloid leukemia cell.
537 The method of any one of Embodiments 525-533, wherein a system is or comprises a small cell lung cancer cell.
538. The method of any one of Embodiments 525-533, wherein a system is or comprises a neuroendocrine prostate cancer cell.
539. The method of any one of Embodiments 525-533, wherein a system is or comprises a tissue.
540. The method of any one of Embodiments 525-533, wherein a system is or comprises an organ.
541. The method of any one of Embodiments 525-533, wherein a system is or comprises a sample.
542. The method of any one of Embodiments 525-533, wherein a system is or comprises an organism.
543. The method of any one of Embodiments 525-533, wherein a system is or comprises a subject.
544. The method of any one of Embodiments 525-533, wherein a system is a human.
545. A method for modulating a dysregulated cell state associated with KAT2A and/or KAT2B, comprising administering or delivering to the cell an effective amount of a compound of any one of the preceding Embodiments, or a pharmaceutically acceptable salt thereof, or a composition of any one of the preceding Embodiments.
546. The method of Embodiment 545, wherein the dysregulated cell state is of cancer.
547. The method of Embodiment 546, wherein the dysregulated call state is of small cell lung cancer.
548. The method of Embodiment 546, wherein the dysregulated call state is of neuroendocrine prostate cancer.
549 The method of Embodiment 545, wherein the dysregulated call state is of acute myeloid leukemia.
550. The method of any one of Embodiments 545-549, wherein the method induces or promotes conversion of the dysregulated cell state into another cell state.
551. The method of any one of Embodiments 545-550, wherein the dysregulated cell state is more proliferative than the another cell state.
552. The method of any one of Embodiments 545-551, wherein the dysregulated cell state is less differentiated than the another cell state.
553. The method of any one of Embodiments 545-552, wherein the dysregulated cell state is or comprises blocked or reduced differentiation.
554. The method of any one of Embodiments 545-553, wherein the dysregulated cell state is or comprises de-differentiation.
555. The method of any one of Embodiments 545-554, wherein the dysregulated cell state is or comprises trans-differentiation.
556. The method of any one of Embodiments 545-555, wherein the dysregulated cell state is or comprises a dedifferentiated neuroendocrine cell state.
557. The method of any one of Embodiments 545-556, wherein the another cell state is or comprises a more differentiated epithelial cell state.
558. The method of any one of Embodiments 545-557, wherein the another cell state comprises increased levels of CD11b expression.
559. The method of any one of Embodiments 545-558, wherein the another cell state comprises increased levels of CD14 expression.
560. The method of any one of Embodiments 545-559, wherein the another cell state comprises increased levels of CD15 expression.
561. The method of any one of Embodiments 545-560, wherein the another cell state comprises increased levels of CD86 expression.
562. The method of any one of Embodiments 545-561, wherein the method increases myeloid differentiation, and/or decreases stem, translation and replication.
563. The method of any one of Embodiments 545-562, wherein the method increases myeloid differentiation, and/or decreases stem, translation and/or replication GSEA signatures.
564. The method of any one of Embodiments 545-563, wherein the method increases epithelial GSEA signatures.
565. The method of any one of Embodiments 545-564, wherein the method increases neural GSEA signatures.
566. The method of any one of Embodiments 545-565, wherein the method increases monocytic differentiation.
567. The method of any one of Embodiments 545-566, wherein the method increases neural differentiation.
568. The method of any one of Embodiments 545-567, wherein the method increases epithelial differentiation.
569. The method of any one of Embodiments 545-568, wherein the method increases level of CD11b⁺ cells.
570. The method of any one of Embodiments 545-569, wherein the method increases level of CD14⁺ cells.
571. The method of any one of Embodiments 545-570, wherein the method increases level of CD15⁺ cells.
572. The method of any one of Embodiments $45-571, wherein the method increases level of CD86⁺ cells.
573. A method, comprising administering a compound of any one of the preceding Embodiments, or a pharmaceutically acceptable salt thereof, or a pharmaceutical composition of any one of the preceding Embodiments, to a subject in need thereof.
574. A method, comprising delivering a compound of any one of the preceding Embodiments, or a pharmaceutically acceptable salt thereof, or a pharmaceutical composition of any one of the preceding Embodiments, to a subject in need thereof.
575. A method of treating a cancer, comprising administering a compound of any one of the preceding Embodiments, or a pharmaceutically acceptable salt thereof, or a pharmaceutical composition of any one of the preceding Embodiments, to a subject in need thereof.
576. A method of treating a cancer, comprising delivering a compound of any one of the preceding Embodiments, or a pharmaceutically acceptable salt thereof, or a pharmaceutical composition of any one of the preceding Embodiments, to a subject in need thereof.
577. The method of Embodiment 575 or 576, wherein the cancer is selected from acute myeloid leukemia, neuroblastoma, non-small cell lung cancer, small cell lung cancer colorectal cancer, melanoma, and prostate cancer
578. A method of degrading KAT2 in a subject, comprising administering a compound of any one of the preceding Embodiments, or a pharmaceutically acceptable salt thereof, or a pharmaceutical composition of any one of the preceding Embodiments, to a subject in need thereof.
579. An in vitro method of degrading KAT2, comprising contacting a biological sample with a compound of any one of the preceding Embodiments, or a pharmaceutically acceptable salt thereof, or a pharmaceutical composition of any one of the preceding Embodiments.
580. A method for treating a disease, disorder, or condition, comprising administering or delivering to a subject suffering from an effective amount of a compound of any one of the preceding Embodiments, or a pharmaceutically acceptable salt thereof, or a pharmaceutical composition of any one of the preceding Embodiments.
581. The method of Embodiment 580, wherein the disease, disorder, or condition is associated with KAT2A.
582. The method of any one of Embodiments 580-581, wherein the disease, disorder, or condition is associated with KAT2B.
583. The method of any one of Embodiments 580-582, wherein the disease, disorder, or condition is cancer.
584. The method of any one of Embodiments 580-582, wherein the disease, disorder, or condition is acute myeloid leukemia.
585. The method of any one of Embodiments 580-582, wherein the disease, disorder, or condition is neuroblastoma.
586. The method of any one of Embodiments 580-582, wherein the disease, disorder, or condition is non-small cell lung cancer.
587. The method of any one of Embodiments 580-582, wherein the disease, disorder, or condition is small cell lung cancer.
588. The method of any one of Embodiments 580-582, wherein the disease, disorder, or condition is colorectal cancer, melanoma.
589. The method of any one of Embodiments 580-582, wherein the disease, disorder, or condition is prostate cancer.
590. The method of any one of Embodiments 580-582, wherein the disease, disorder, or condition is neuroendocrine prostate cancer.
591. The method of any one of Embodiments 580-582, wherein the disease, disorder, or condition is castration resistant prostate cancer.
592. The method of any one of Embodiments 580-582, wherein the disease, disorder, or condition is high-grade neuroendocrine carcinomas.
593. The method of any one of Embodiments 580-582, wherein the disease, disorder, or condition is high-grade neuroendocrine carcinomas in GI tract.
594. The method of any one of Embodiments 580-582, wherein the disease, disorder, or condition is high-grade neuroendocrine carcinomas in pancreas.
595. The method of any one of Embodiments 580-582, wherein the disease, disorder, or condition is pancreatic cancer.
596. The method of any one of Embodiments 580-595, comprising a method of any one of Embodiments 526-573.
597. A compound of Formula II:

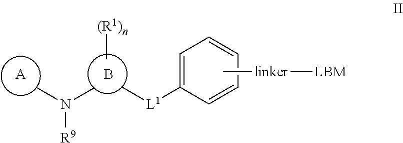

or a pharmaceutically acceptable salt thereof, wherein:
Ring A is selected from:

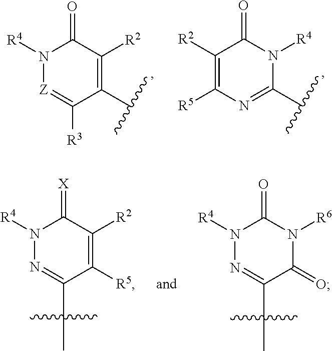

Ring B is a 5- to 6-membered heterocyclyl having 1-2 heteroatoms independently selected from N, O, and S;
L¹ is a covalent bond or a bivalent C₁₋₃ straight or branched hydrocarbon chain;
each R¹ is independently optionally substituted C₁₋₆ aliphatic or optionally substituted C₃₋₆ cycloaliphatic;
n is 0, 1, 2, 3, or 4;
Z is Nor CR³;
R² is hydrogen, halogen, —CN, optionally substituted C₁₋₆ aliphatic, or optionally substituted C₃₋₆ cycloaliphatic;
each R³ is independently hydrogen, halogen, optionally substituted C₁₋₆ aliphatic, or optionally substituted C₃₋₆ cycloaliphatic;
R⁴ is hydrogen, optionally substituted C₁₋₆ aliphatic, or optionally substituted C₃₋₆ cycloaliphatic;
R⁵ is hydrogen, halogen, optionally substituted C₁₋₆ aliphatic, or optionally substituted C₃₋₆ cycloaliphatic, or
R² and R⁵, together with the atoms to which they are attached, combine to form an optionally substituted 5- to 6-membered aromatic ring having 0-2 heteroatoms independently selected from N, O, and S;
R⁶ is hydrogen, optionally substituted C₁₋₆ aliphatic, or optionally substituted C₃₋₆ cycloaliphatic;
X is O or NR⁷;
R⁷ is hydrogen or optionally substituted C₁₋₆ aliphatic, or
R⁴ and R⁷, together with the atoms to which they are attached, combine to form an optionally substituted 5- to 6-membered aromatic ring having 2-3 heteroatoms independently selected from N, O, and S;
R⁹ is hydrogen or optionally substituted C₁₋₆ aliphatic;
linker is a covalent bond or an optionally substituted, bivalent, straight or branched, saturated or unsaturated C₁-C₂ hydrocarbon chain, wherein one or more methylene units are optionally and independently replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —SO₂—, —SO₂N(R)—, —N(R)SO₂—, or -Cy-;
each Cy is independently an optionally substituted, mono- or multicyclic, 3- to 16-membered bivalent ring system, wherein the ring system is fully saturated, partially saturated, or aromatic, and the ring system contains 0-6 heteroatoms independently selected from N, O, and S;
each R is independently hydrogen or an optionally substituted group selected from C₁₋₆ aliphatic, phenyl, C₃₋₇ cycloaliphatic, 5- to 6-membered monocyclic heteroaryl having 1-4 heteroatoms independently selected from N, O, and S, and a 3- to 7-membered monocyclic heterocyclyl having 1-2 heteroatoms independently selected from N, O, and S; and
LBM is an E3 ubiquitin ligase binding moiety,
wherein the compound is not:

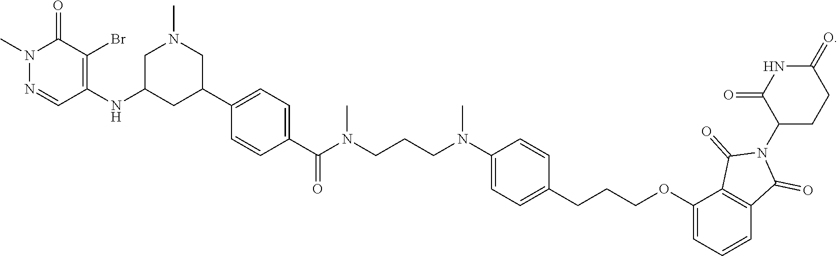 [SMILES: CN1CC(Nc2cnn(C)c(=O)c2Br)CC(c2ccc(C(=O)N(C)CCCN(C)c3ccc(CCCOc4cccc5c4C(=O)N(C4CCC(=O)NC4=O)C5=O)cc3)cc2)C1]

598. The compound of Embodiment 597, wherein Ring A is

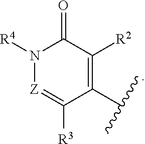

599. The compound of Embodiment 598, wherein Ring A is selected from:

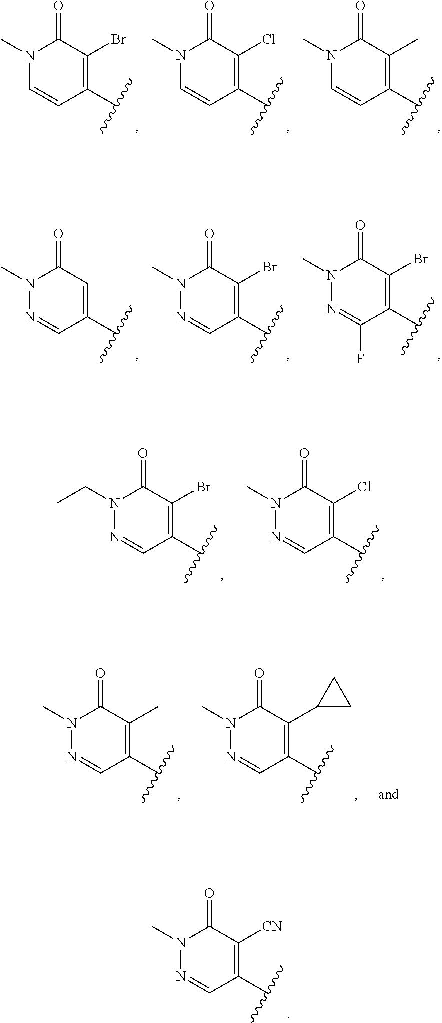

600. The compound of Embodiment 599, wherein Ring A is selected from:

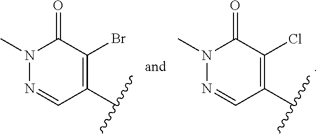

601. The compound of Embodiment 597, wherein Ring A is

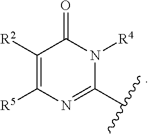

602. The compound of Embodiment 601, wherein R² and R⁵, together with the atoms to which they are attached, combine to form an optionally substituted 5- to 6-membered aromatic ring having 0-2 heteroatoms independently selected from N, O, and S.
603. The compound of Embodiment 602, wherein Ring A is

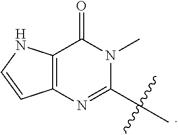 [SMILES: *c1nc2cc[nH]c2c(=O)n1C]

604 The compound of Embodiment 597, wherein Ring A is

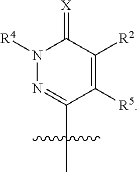

605. The compound of Embodiment 604, wherein R² and R⁵, together with the atoms to which they are attached, combine to form an optionally substituted 5- to 6-membered aromatic ring having 0-2 heteroatoms independently selected from N, O, and S.
606. The compound of Embodiment 605, wherein Ring A is selected from:

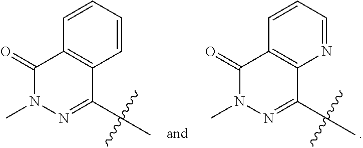 [SMILES: *c1nn(C)c(=O)c2ccccc12 ; *c1nn(C)c(=O)c2cccnc12]

607. The compound of Embodiment 605, wherein X is NR⁷, and R⁴ and R⁷, together with the atoms to which they are attached, combine to form an optionally substituted 5- to 6-membered aromatic ring having 2-3 heteroatoms independently selected from N, O, and S.
608. The compound of Embodiment 607, wherein Ring A is

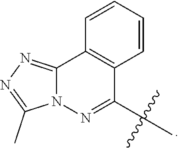 [SMILES: *c1nn2c(C)nnc2c2ccccc12]

609. The compound of Embodiment 597, wherein Ring A is

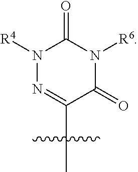

610. The compound of Embodiment 609, wherein Ring A is

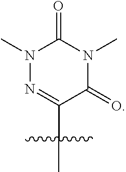

611. The compound of any one of Embodiments 597-610, wherein R⁹ is hydrogen.
612. The compound of any one of Embodiments 597-611, wherein Ring B is a 6-membered heterocyclyl having 1-2 heteroatoms independently selected from N, O, and S.
613. The compound of Embodiment 612, wherein Ring B is

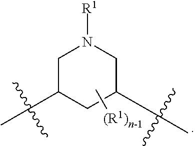

614. The compound of Embodiment 613, wherein Ring B is

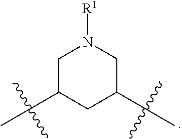

615. The compound of any one of Embodiments 597-614, wherein L¹ is a covalent bond.
616. The compound of any one of Embodiments 597-615, wherein each R¹ is independently optionally substituted C₁₋₆ alkyl.
617. The compound of Embodiment 616, wherein each R¹ is independently C₁₋₆ alkyl.
618. The compound of any one of Embodiments 597-617, wherein n is 0 or 1.
619. The compound of any one of Embodiments 597-618, wherein R² is halogen or C₁₋₆ alkyl.
620. The compound of any one of Embodiments 597-619, wherein R² is halogen.
621. The compound of any one of Embodiments 597-620, wherein each R³ is hydrogen.
622. The compound of any one of Embodiments 597-621, wherein R⁴ is optionally substituted C₁₋₆ alkyl.
623. The compound of any one of Embodiment 597, wherein the moiety

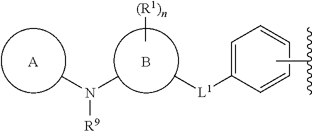

is selected from:

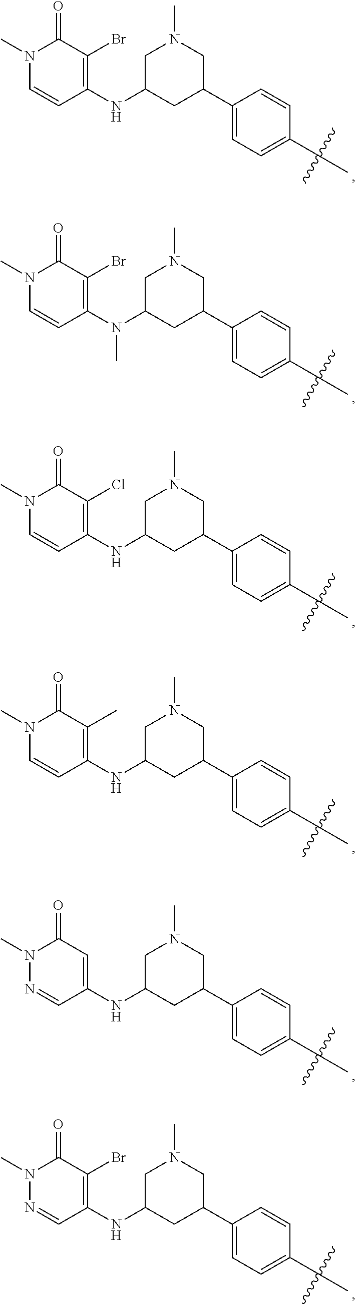 [SMILES: *c1ccc(C2CC(Nc3ccn(C)c(=O)c3Br)CN(C)C2)cc1 ; *c1ccc(C2CC(N(C)c3ccn(C)c(=O)c3Br)CN(C)C2)cc1 ; *c1ccc(C2CC(Nc3ccn(C)c(=O)c3Cl)CN(C)C2)cc1 ; *c1ccc(C2CC(Nc3ccn(C)c(=O)c3C)CN(C)C2)cc1 ; *c1ccc(C2CC(Nc3cnn(C)c(=O)c3)CN(C)C2)cc1 ; *c1ccc(C2CC(Nc3cnn(C)c(=O)c3Br)CN(C)C2)cc1]

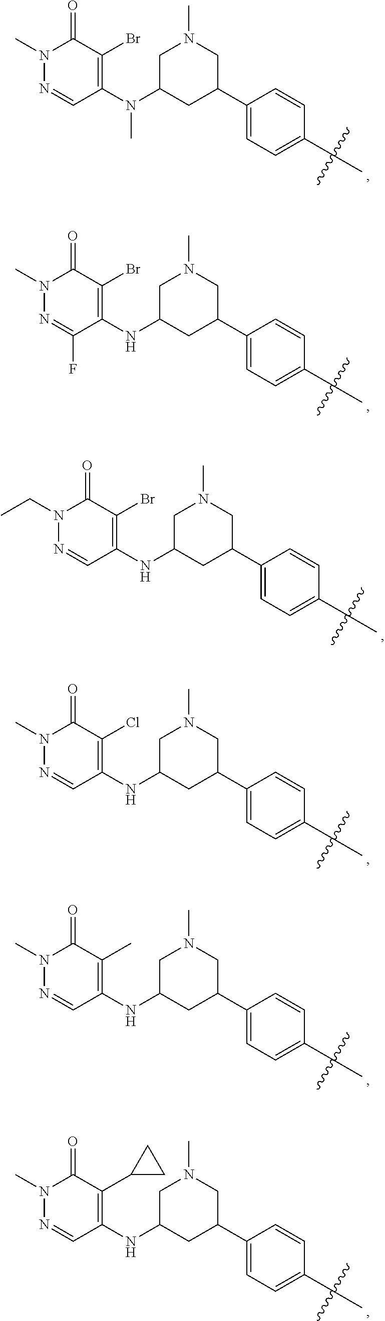 [SMILES: *c1ccc(C2CC(N(C)c3cnn(C)c(=O)c3Br)CN(C)C2)cc1 ; *c1ccc(C2CC(Nc3c(F)nn(C)c(=O)c3Br)CN(C)C2)cc1 ; *c1ccc(C2CC(Nc3cnn(CC)c(=O)c3Br)CN(C)C2)cc1 ; *c1ccc(C2CC(Nc3cnn(C)c(=O)c3Cl)CN(C)C2)cc1 ; *c1ccc(C2CC(Nc3cnn(C)c(=O)c3C)CN(C)C2)cc1 ; *c1ccc(C2CC(Nc3cnn(C)c(=O)c3C3CC3)CN(C)C2)cc1]

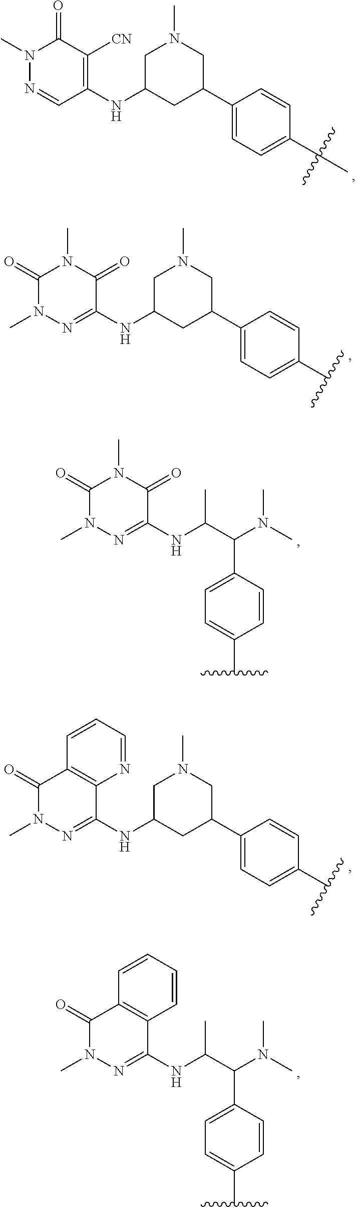 [SMILES: *c1ccc(C2CC(Nc3cnn(C)c(=O)c3C#N)CN(C)C2)cc1 ; *c1ccc(C2CC(Nc3nn(C)c(=O)n(C)c3=O)CN(C)C2)cc1 ; *c1ccc(C(C(C)Nc2nn(C)c(=O)n(C)c2=O)N(C)C)cc1 ; *c1ccc(C2CC(Nc3nn(C)c(=O)c4cccnc34)CN(C)C2)cc1 ; *c1ccc(C(C(C)Nc2nn(C)c(=O)c3ccccc23)N(C)C)cc1]

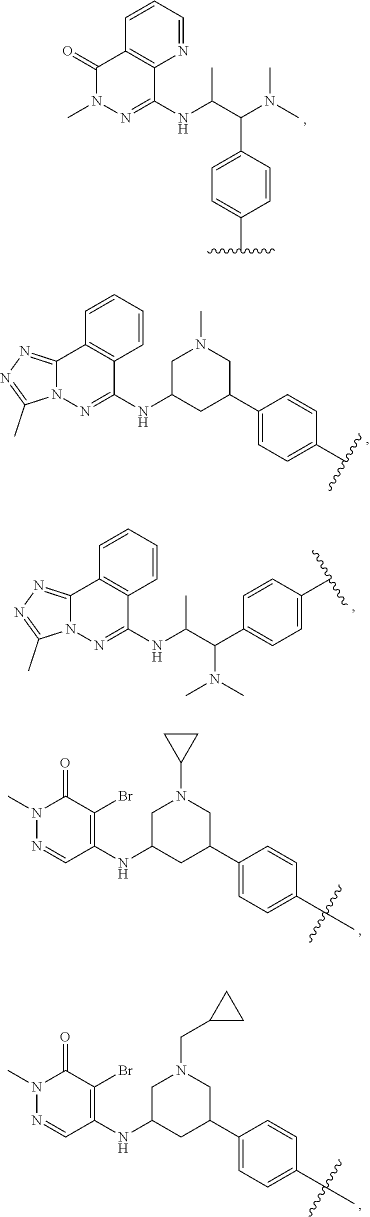

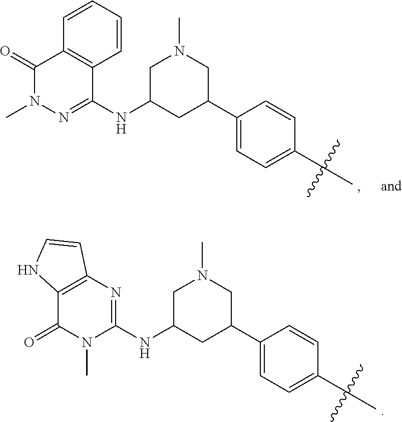 [SMILES: *c1ccc(C2CC(Nc3nn(C)c(=O)c4ccccc34)CN(C)C2)cc1 ; *c1ccc(C2CC(Nc3nc4cc[nH]c4c(=O)n3C)CN(C)C2)cc1]

624. The compound of any one of Embodiments 597-623, wherein linker is an optionally substituted, bivalent, straight or branched, saturated or unsaturated C₁-C₂₀ hydrocarbon chain, wherein one or more methylene units are optionally and independently replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —SO₂—, —SO₂N(R)—, —N(R)SO₂—, or -Cy-.
625. The compound of Embodiment 624, wherein linker is an optionally substituted, bivalent, straight or branched, saturated or unsaturated C₁-C₁₀ hydrocarbon chain, wherein one or more methylene units are optionally and independently replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —SO₂—, —SO₂N(R)—, —N(R)SO₂—, or -Cy-.
626. The compound of Embodiment 624, wherein linker is an optionally substituted, bivalent, straight or branched, saturated or unsaturated C₁-C₂₀ hydrocarbon chain, wherein at least one methylene unit is replaced by —O—, —N(R)—, —C(O)—, —OC(O)—, —C(O)O—, —C(O)N(R)—, —N(R)C(O)—, or -Cy-.
627. The compound of Embodiment 626, wherein linker is an optionally substituted, bivalent, straight or branched, saturated or unsaturated C₁-C₂₀ hydrocarbon chain, wherein at least two methylene units are replaced by —O—, —N(R)—, —C(O)—, —OC(O)—, —C(O)O—, —C(O)N(R)—, —N(R)C(O)—, or -Cy-.
628. The compound of any one of Embodiments 624-627, wherein linker is an optionally substituted, bivalent, straight or branched, saturated or unsaturated C₁-C₂₀ hydrocarbon chain, wherein at least one methylene unit is replaced by —C(O)N(R)—.
629. The compound of any one of Embodiments 624-628, wherein linker comprises at least one triple bond.
630. The compound of any one of Embodiments 624-629, wherein linker is an optionally substituted, bivalent, straight or branched, saturated or unsaturated C₁-C₂₀ hydrocarbon chain, wherein at least one methylene unit is replaced by -Cy-.
631. The compound of any one of Embodiments 597-630, wherein linker is:

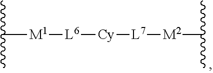

wherein:
M¹ and M² are each independently absent, —CH₂—, —O—, —N(R)—, —C(O)—, —OC(O)—, —C(O)O—, —C(O)N(R)—, or —N(R)C(O)—; and
L⁶ and L⁷ are each independently a covalent bond or an optionally substituted, bivalent, straight or branched, saturated or unsaturated C₁-C₁₀ hydrocarbon chain, wherein one or more methylene units are optionally and independently replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —SO₂—, —SO₂N(R)—, —N(R)SO₂—, or -Cy-.
632. The compound of Embodiment 631, wherein linker is selected from:

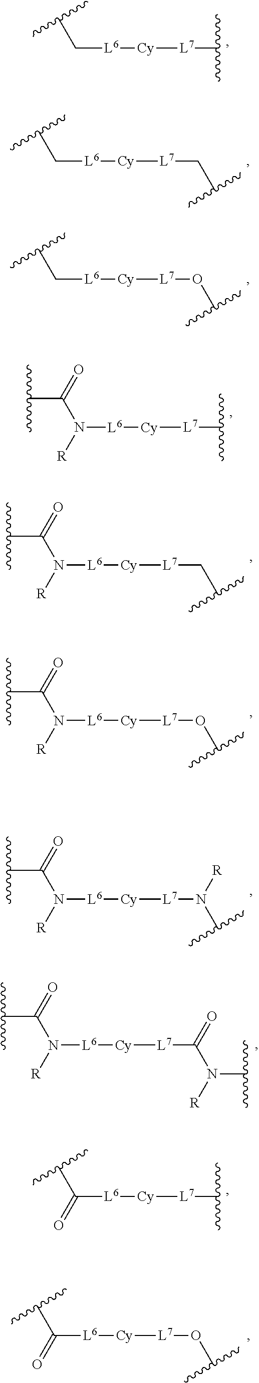

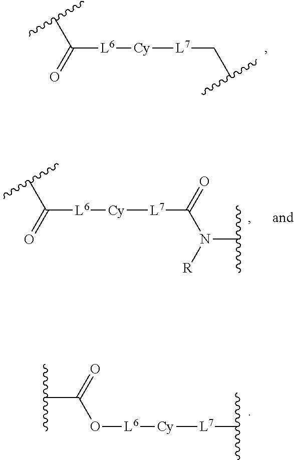

633. The compound of Embodiment 632, wherein linker is

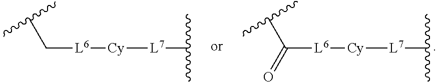

634. The compound of Embodiment 631, wherein linker is selected from:

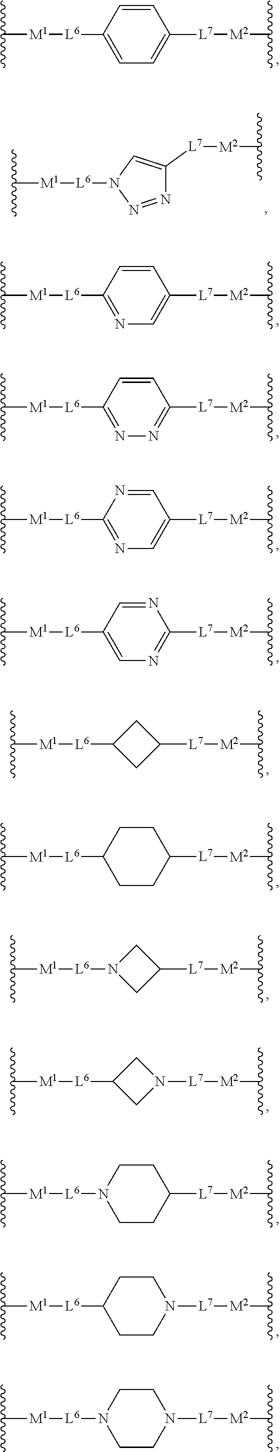

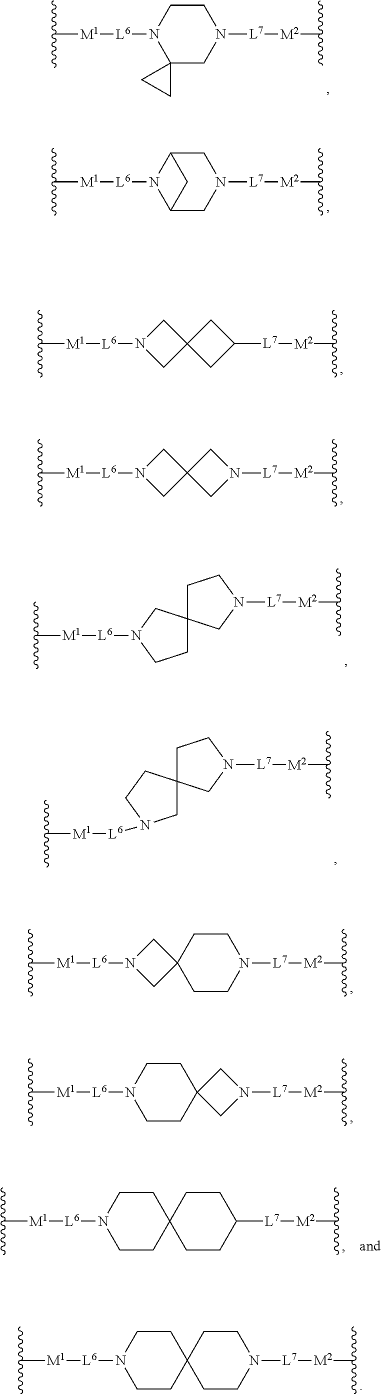

635. The compound of any one of Embodiments 631-634, wherein L⁶ is a covalent bond.
636. The compound of any one of Embodiments 631-635, wherein L⁷ is a covalent bond.
637. The compound of any one of Embodiments 624-636, wherein each Cy is independently an optionally substituted group selected from phenyl, 5- to 6-membered monocyclic heteroaryl having 1-4 heteroatoms independently selected from N, O, and S, monocyclic 4- to 7-membered heterocyclyl having 1-2 heteroatoms independently selected from N, O, and S, and bicyclic 6- to 11-membered heterocyclyl having 1-3 heteroatoms independently selected from N, O, and S.
638. The compound of Embodiment 637, wherein Cy is optionally substituted phenyl or 5- to 6-membered heteroaryl having 1-4 heteroatoms independently selected from N, O, and S.
639. The compound of Embodiment 637, wherein Cy is optionally substituted monocyclic 4- to 7-membered heterocyclyl having 1-2 heteroatoms independently selected from N, O, and S.
640. The compound of Embodiment 637, wherein Cy is optionally substituted bicyclic 6- to 11-membered heterocyclyl having 1-3 heteroatoms independently selected from N, O, and S.
641. The compound of Embodiment 640, wherein Cy is a spirocyclic 6- to 7-membered heterocyclyl having 1-3 heteroatoms independently selected from N, O, and S.
642. The compound of any one of Embodiments 624-636, wherein each Cy is independently selected from:

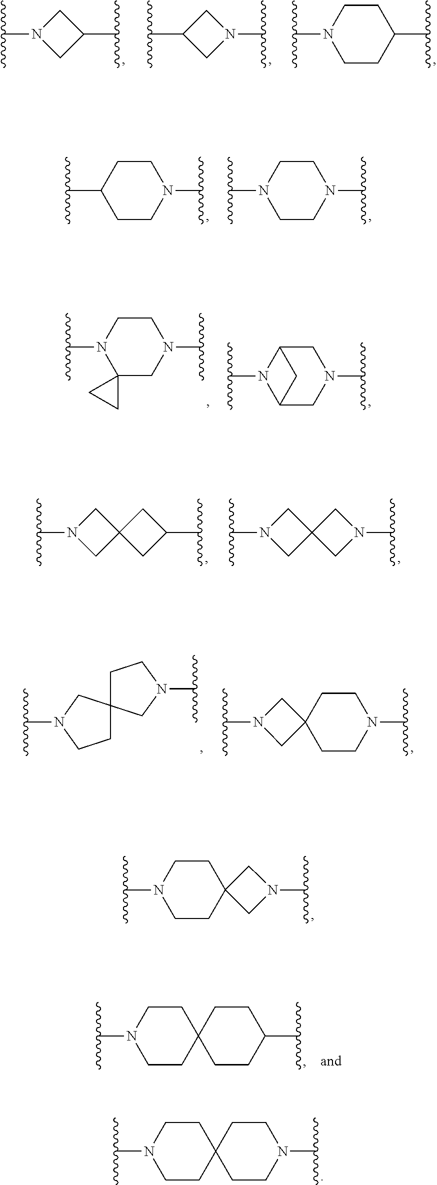

643. The compound of any one of Embodiments 597-642, wherein each R is independently hydrogen or optionally substituted C₁₋₆ alkyl.
644. The compound of any one of Embodiment 624, wherein linker is selected from: —CH₂—,

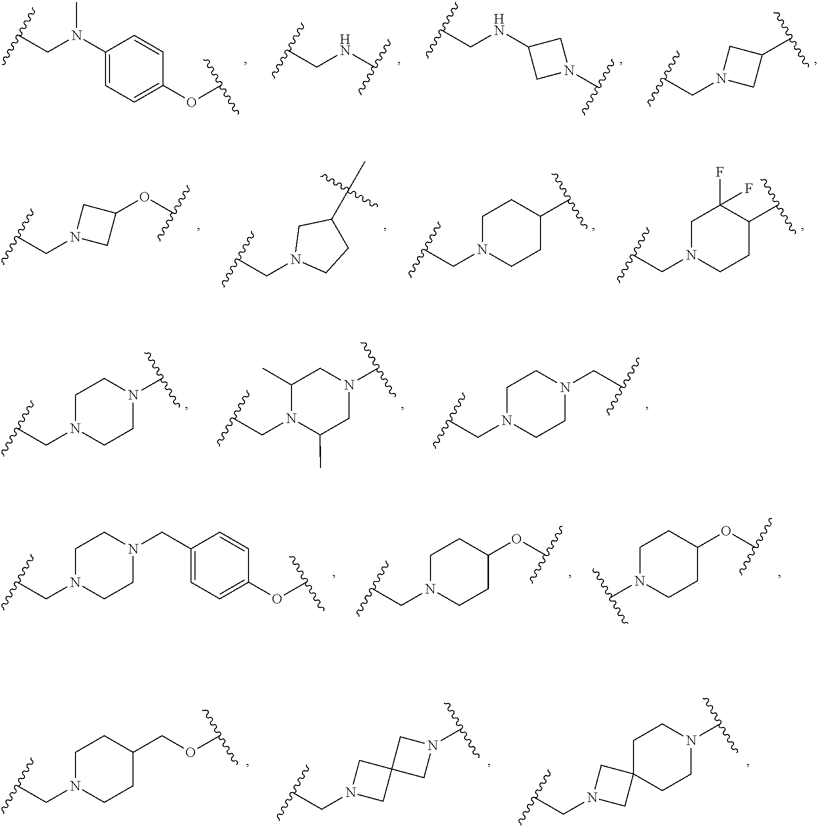

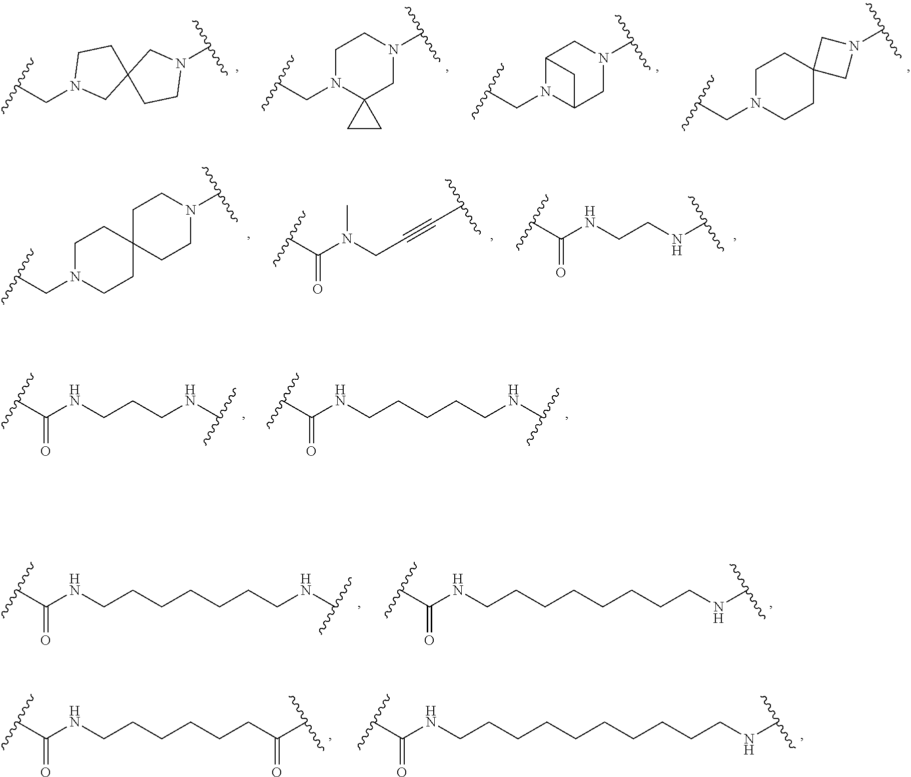

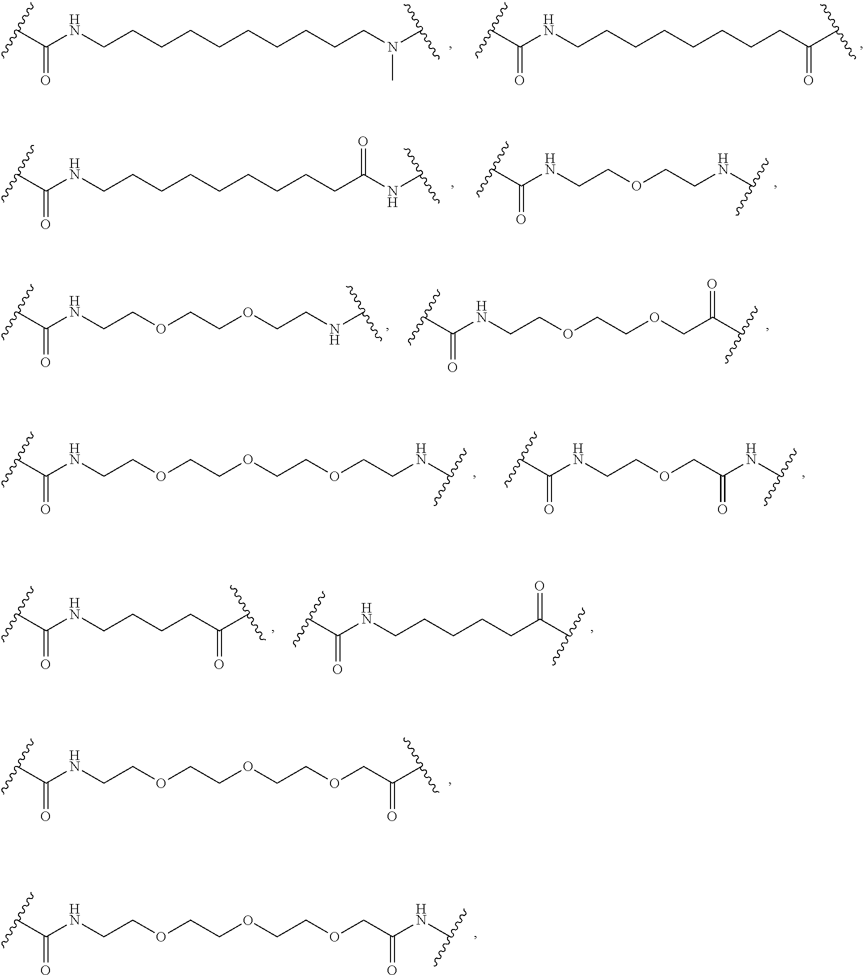

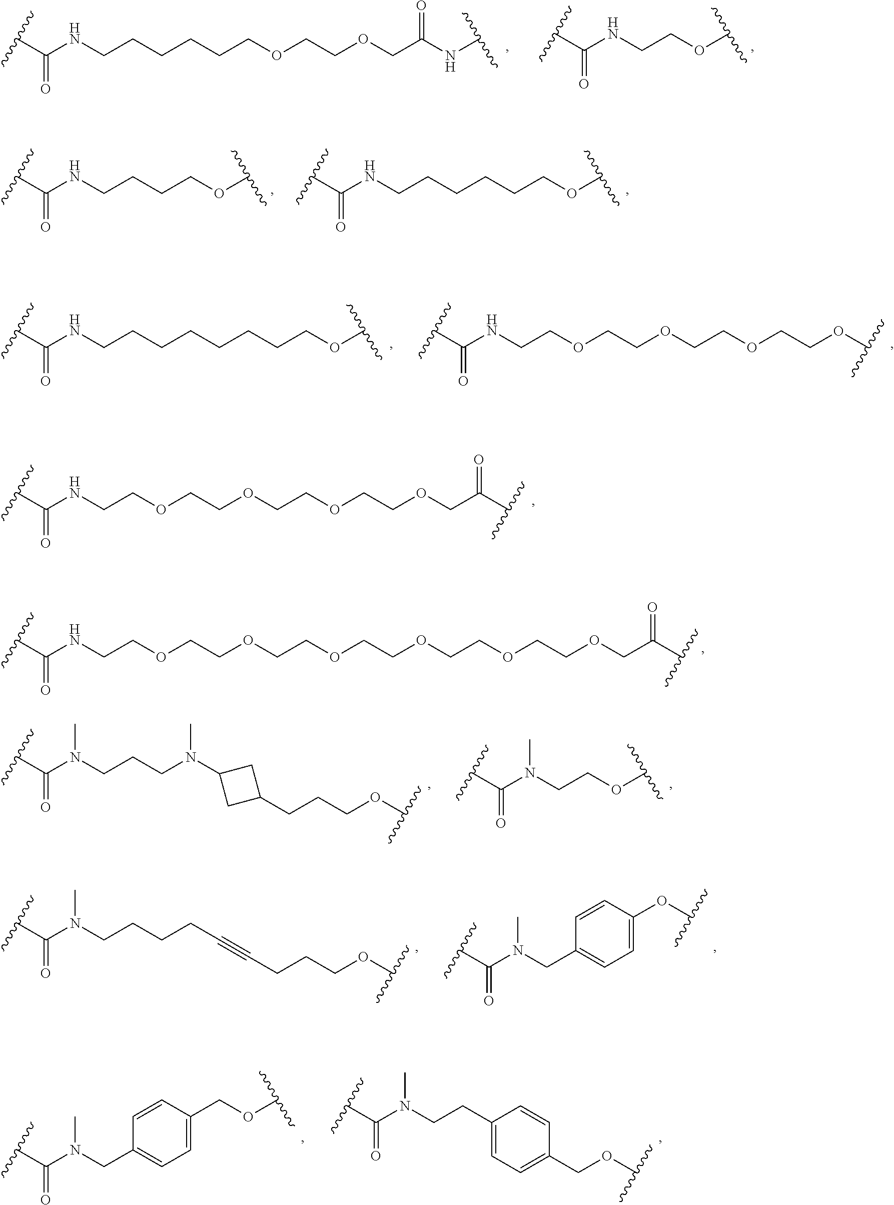

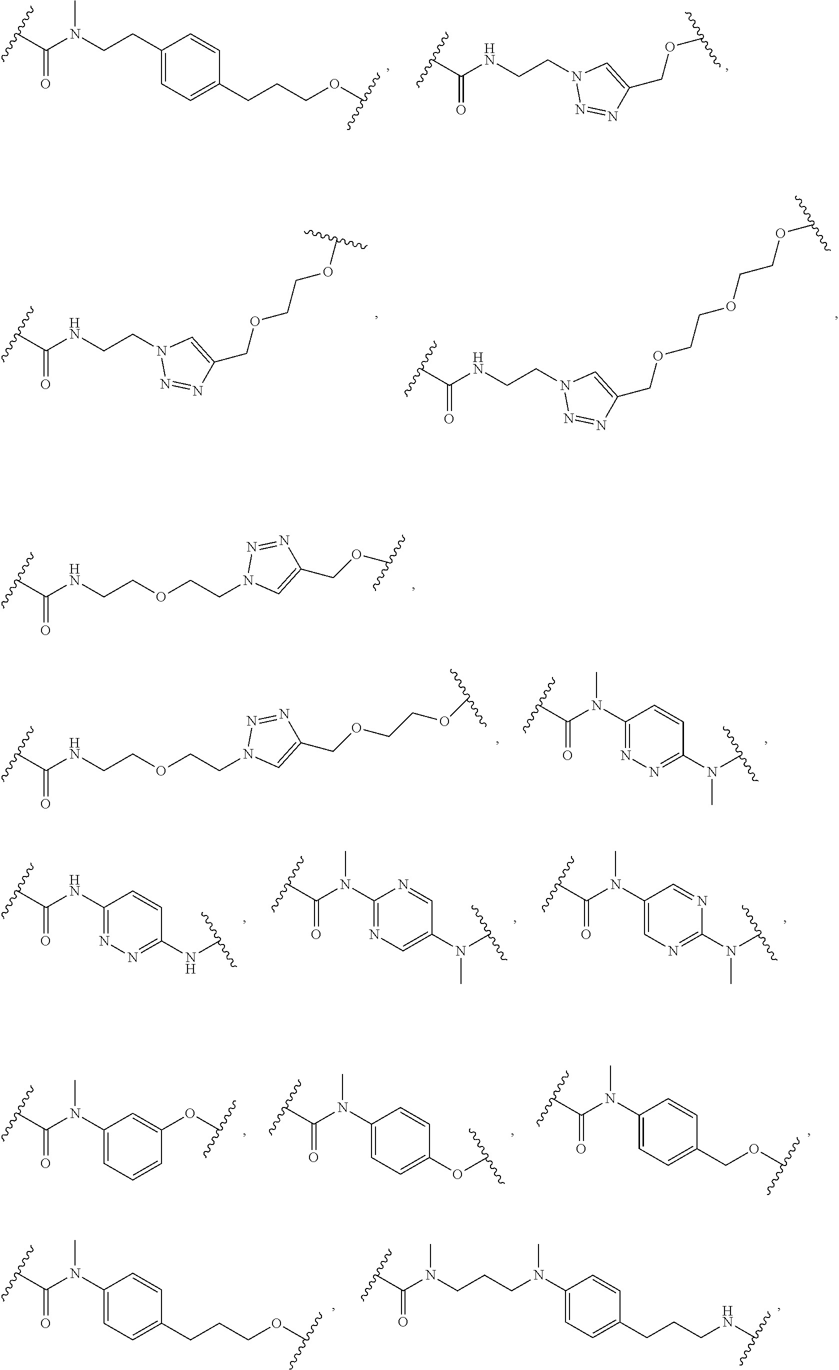

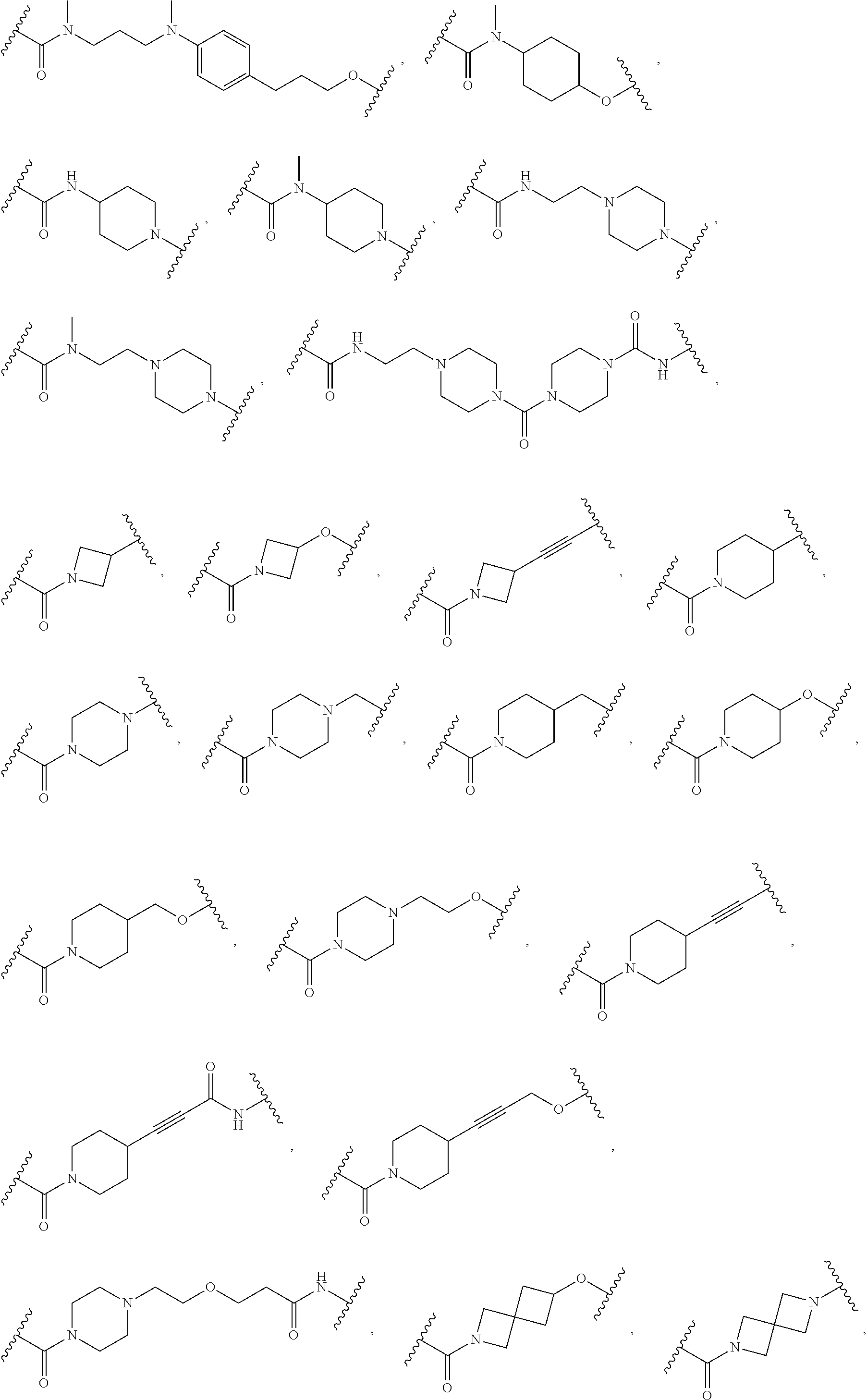

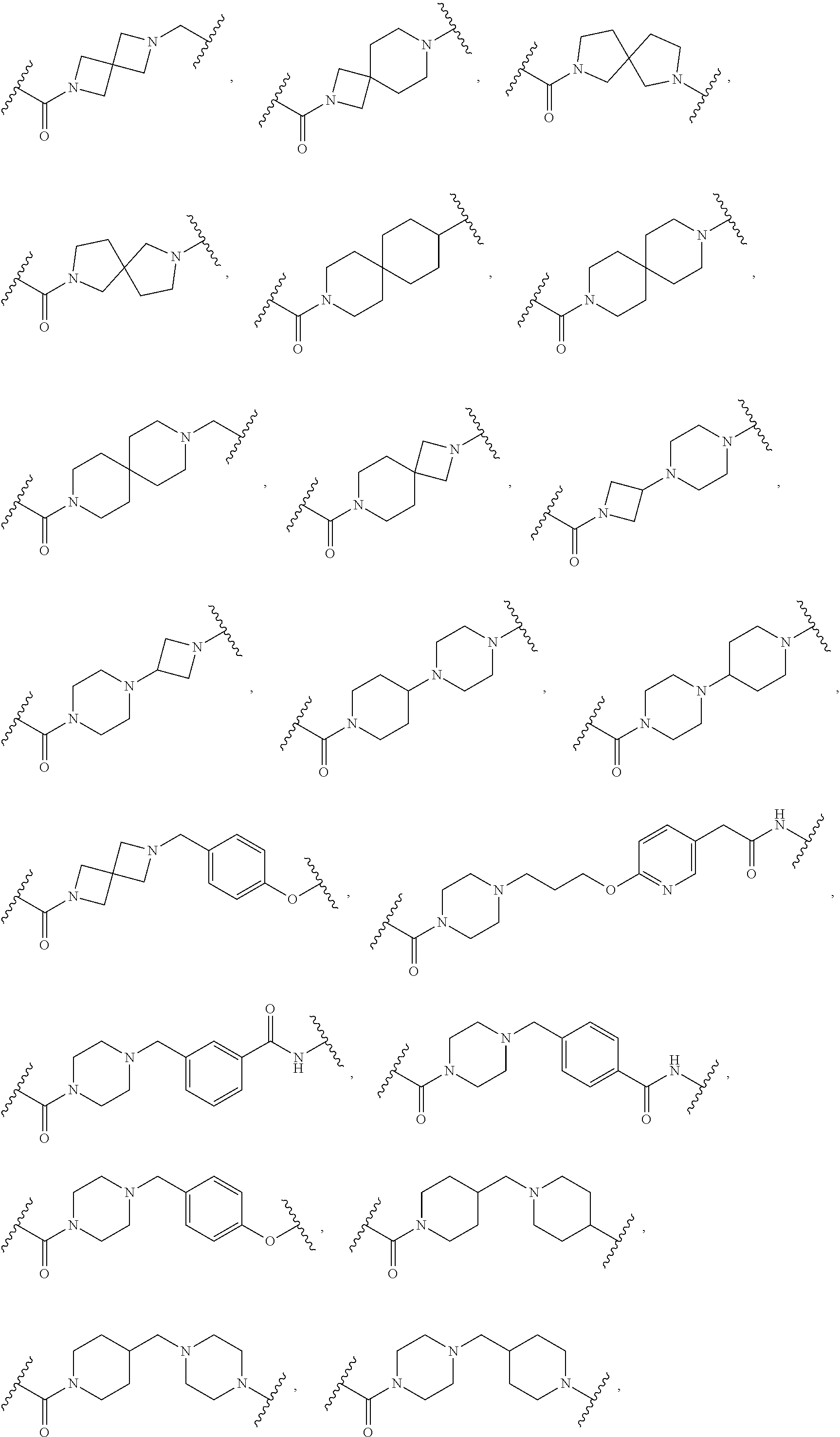

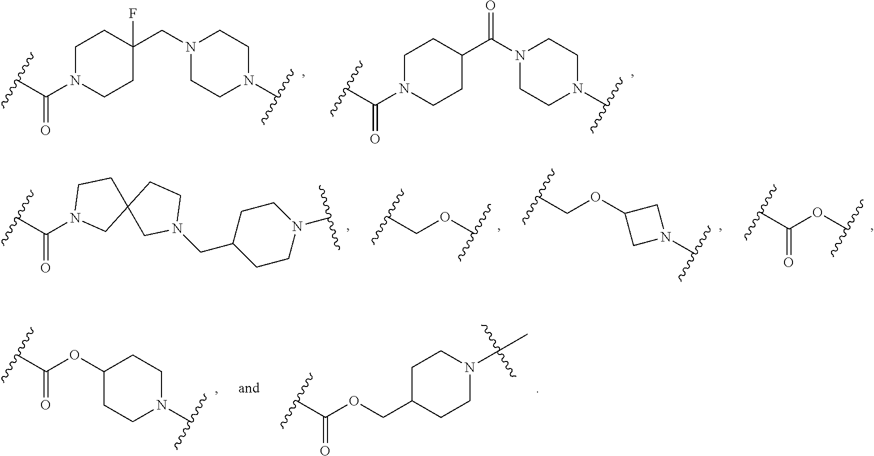

645. The compound of any one of Embodiments 597-644, wherein LBM is a CRBN binding moiety.
646. The compound of any one of Embodiments 597-644, wherein LBM is a VHL binding moiety.
647. The compound of any one of Embodiments 597-644, wherein LBM is an IAP binding moiety.
648. The compound of any one of Embodiments 597-644, wherein LBM is a MDM2 binding moiety.
649. The compound of any one of Embodiments 597-648, wherein the compound is of Formula IIA:

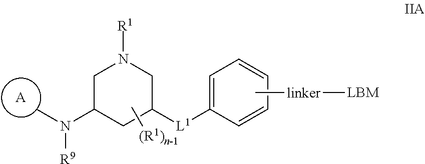

or a pharmaceutically acceptable salt thereof.
650. The compound of Embodiment 649, wherein the compound is of Formula HA-1, HA-2, HA-3, or IIA-4:

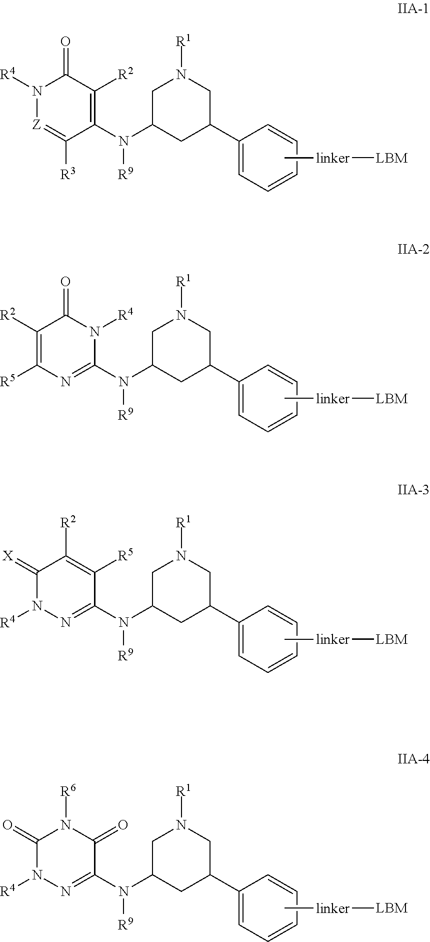

or a pharmaceutically acceptable salt thereof.
651. The compound of any one of Embodiments 597-650, wherein the compound is of Formula IIA-5:

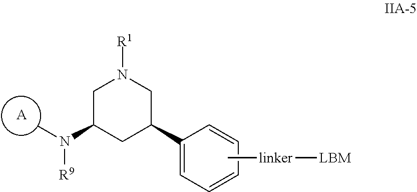

or a pharmaceutically acceptable salt thereof.
652. The compound of any one of Embodiments 597-650, wherein the compound is of Formula IIA-6:

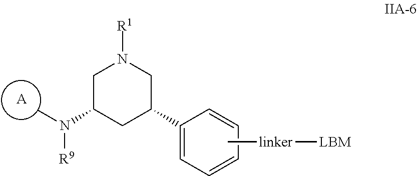

or a pharmaceutically acceptable salt thereof.
653. The compound of any one of Embodiments 597-652, wherein the compound is of Formula III:

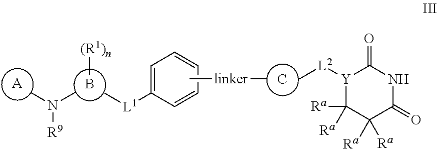

or a pharmaceutically acceptable salt thereof, wherein:
Ring C is an optionally substituted, mono- or multicyclic, 3- to 16-membered bivalent ring system, wherein the ring system is fully saturated, partially saturated, or aromatic, and the ring system contains 0-6 heteroatoms independently selected from N, O, and S;
each R^{a} is independently hydrogen or an optionally substituted C₁₋₆ aliphatic, or two R^{a} groups, together with the atom(s) to which they are attached, combine to form a 3- to 6-membered saturated or partially unsaturated ring;
L² is a covalent bond or a straight or branched C₁₋₃ hydrocarbon chain wherein one methylene is optionally replaced with —O—, —S—, —N(R)—, —SO₂—, —C(O)N(R)—, or —N(R)C(O)—; and
Y is N or CH.
654. The compound of Embodiment 653, wherein Ring C is an optionally substituted group selected from Phenyl, C₅₋₆ cycloaliphatic, 5- to 6-membered heteroaryl having 1-4 heteroatoms independently selected from N, O, and S, 5- to 6-membered heterocyclyl having 1-2 heteroatoms independently selected from N, O, and S, and 9- to 10-membered bicyclic heteroaryl having 1-4 heteroatoms independently selected from N, O, and S.
655. The compound of Embodiment 654, wherein Ring C is optionally substituted phenyl or 5- to 6-membered heteroaryl having 1-4 heteroatoms independently selected from N, O, and S.
656. The compound of Embodiment 655, wherein Ring C is

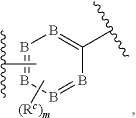

wherein:
   each B is independently selected from N, C, and CH, provided that no more than two B are N;
   each R^{c} is independently selected from halogen, —OR, —N(R)₂, —CN, and optionally substituted C₁₋₆ aliphatic; and
   m is 0, 1, 2, or 3.
657. The compound of Embodiment 656, wherein Ring C is

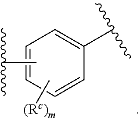

658. The compound of any one of Embodiments 653-657, wherein the compound is of Formula IIIC:

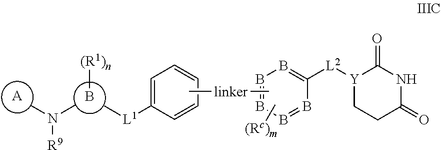

or a pharmaceutically acceptable salt thereof.
659. The compound of any one of Embodiments 653-655, wherein the compound is of Formula IIID:

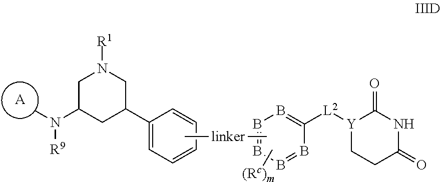

or a pharmaceutically acceptable salt thereof.
660. The compound of Embodiment 653, wherein Ring C is optionally substituted C₃-C₇ cycloaliphatic or 3- to 7-membered heterocyclyl having 1-2 heteroatoms independently selected from N, O, and S.
661. The compound of Embodiment 653, wherein Ring C is optionally substituted 9- to 10-membered bicyclic heteroaryl having 1-4 heteroatoms independently selected from N, O, and S.
662. The compound of Embodiment 661, wherein Ring C is:

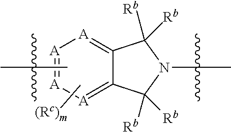

wherein:
each A is independently N, C, or CH, provided that no more than two A groups are N;
each R^{b} is hydrogen, or two R^{b} groups, on the same carbon, are taken together to form an oxo or combine to form a 3- to 6-membered saturated or partially unsaturated ring;
each R^{e} is independently selected from halogen, —OR, —N(R)₂, —CN, and optionally substituted C₁₋₆ aliphatic; and
m is 0, 1, 2, or 3.
663. The compound of Embodiment 662, wherein Ring C is:

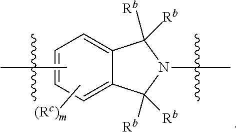

664. The compound of any one of Embodiments 653-663, wherein L² is a covalent bond.
665. The compound of any one of Embodiments 653-663, wherein L² is —CH₂—, —O—, or —N(R)—.
666. The compound of any one of Embodiments 653-665, wherein Y is N.
667. The compound of any one of Embodiments 653-665, wherein Y is CH.
668. The compound of any one of Embodiments 653-667, wherein each R^{e} is independently selected from halogen, —O(C₁₋₆ alkyl), —O(C₁₋₆ haloalkyl), C₁₋₆ alkyl, and C₁₋₆ haloalkyl.
669. The compound of any one of Embodiments 661-668, wherein the compound is of Formula IIIA:

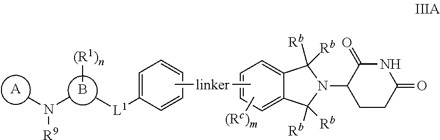

or a pharmaceutically acceptable salt thereof.
670. The compound of Embodiment 669, wherein the compound is of Formula IIIB:

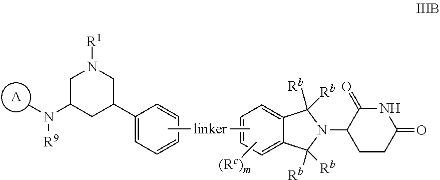

or a pharmaceutically acceptable salt thereof
671. The compound of Embodiment 670, wherein the compound is of Formula IIIB-1 or IIIB-2:

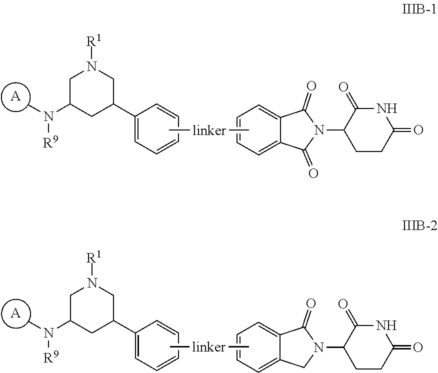

or a pharmaceutically acceptable salt thereof.
672. The compound of Embodiment 653, wherein the

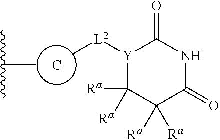

moiety is selected

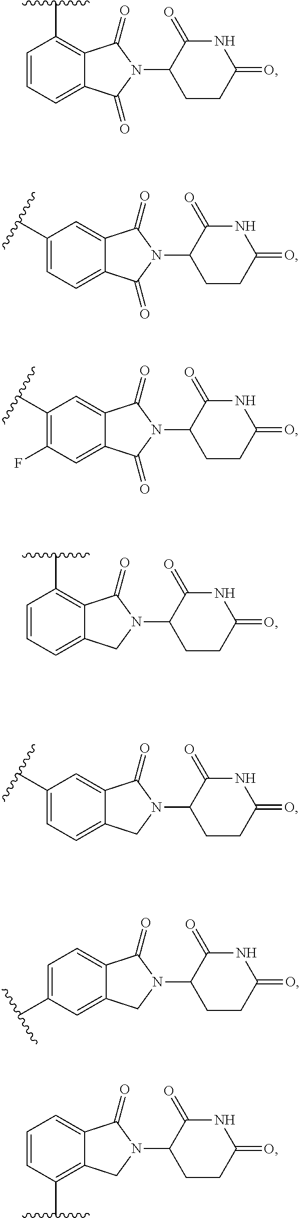

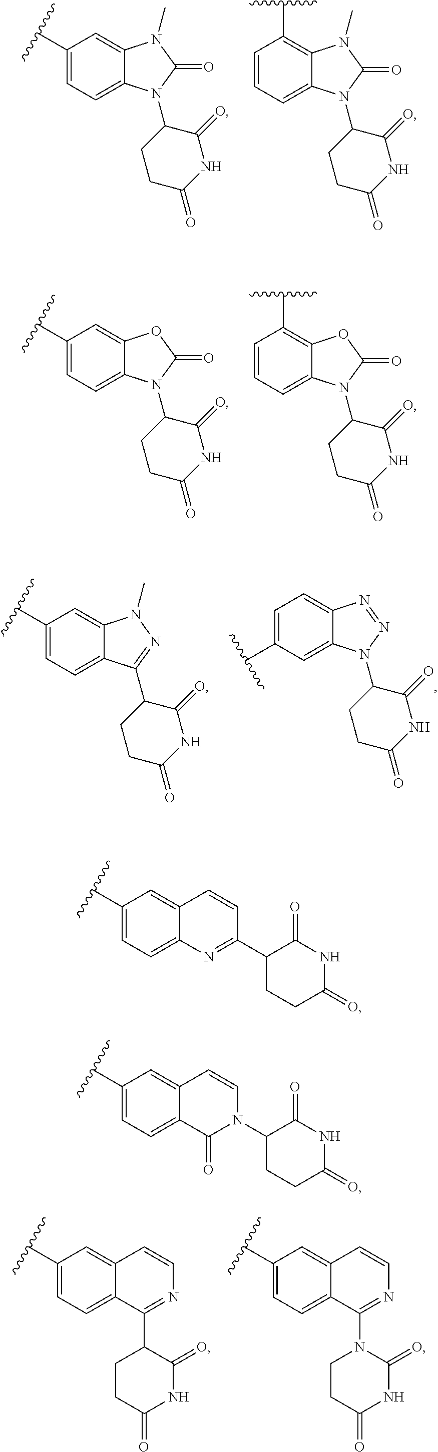

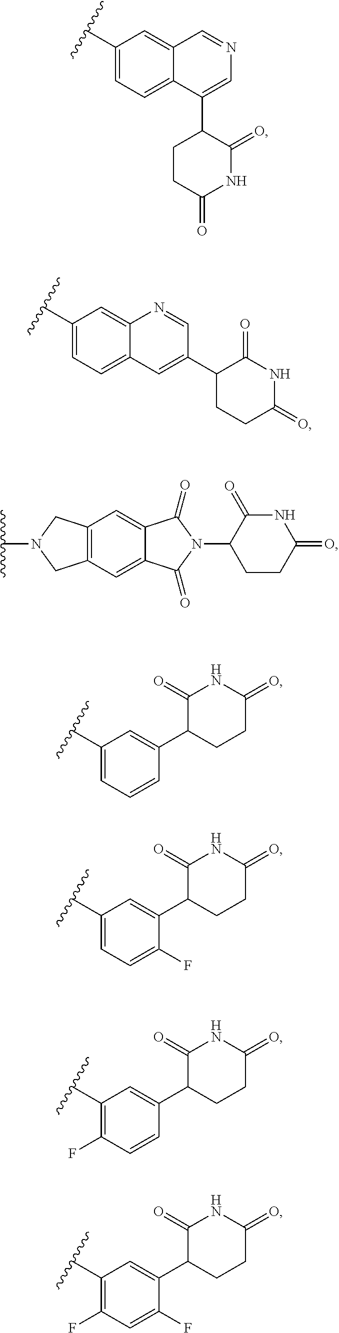

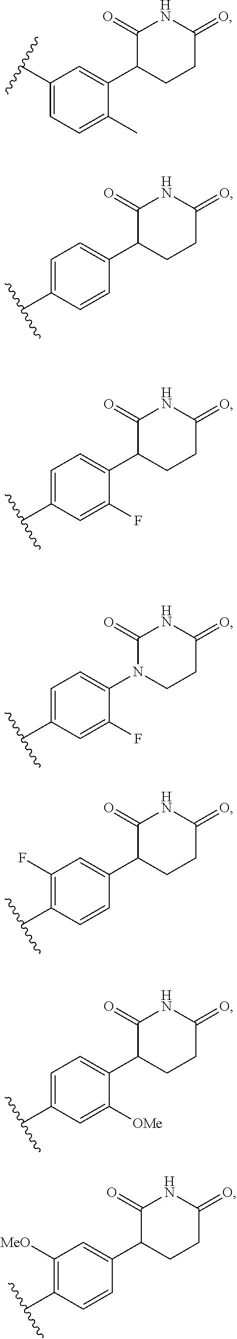

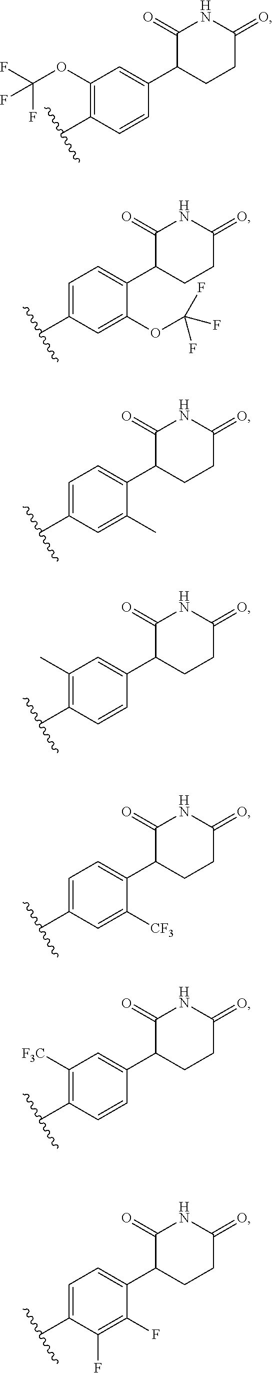

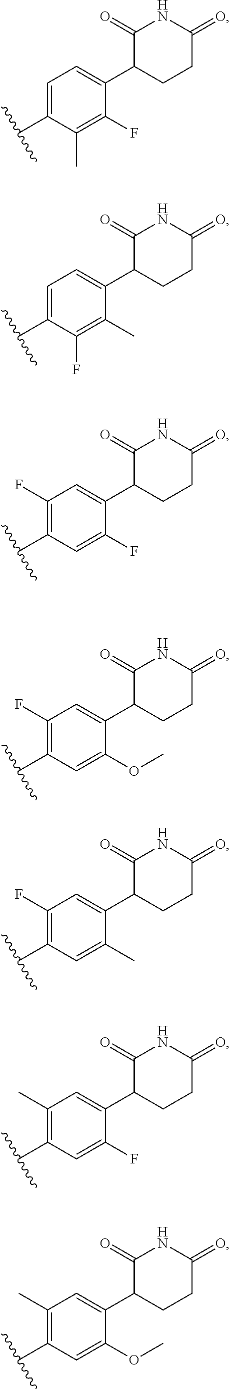

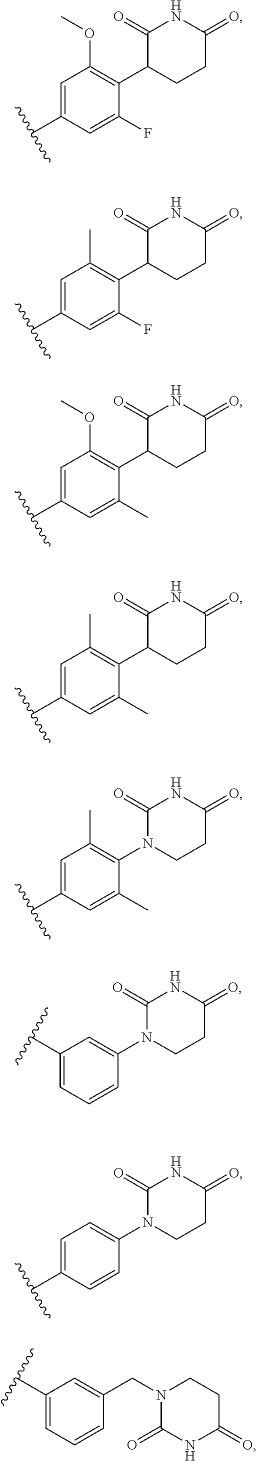

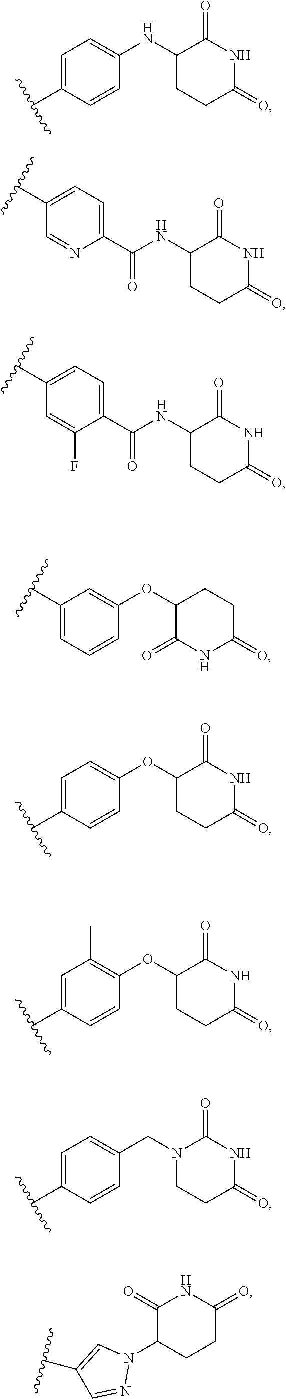

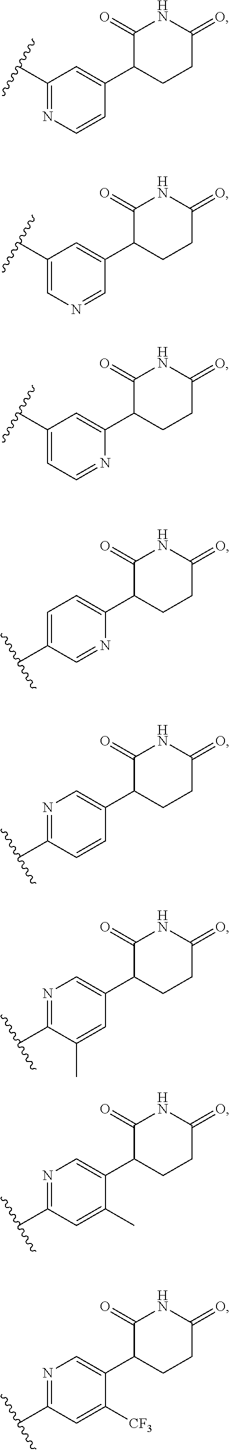

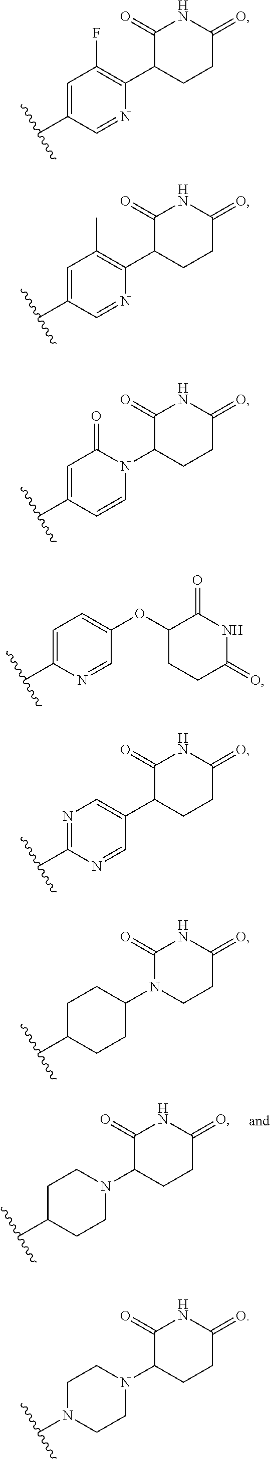

673. A compound of Formula IX:

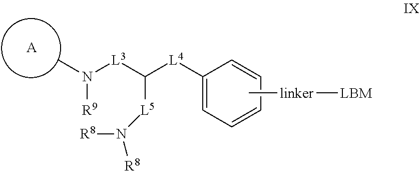

or a pharmaceutically acceptable salt thereof, wherein:
Ring A is selected from:

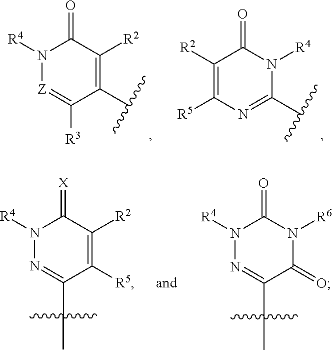

L³, L⁴, and L⁵ are each independently a covalent bond or an optionally substituted bivalent C₁₋₆ straight or branched hydrocarbon chain;
Z is N or CR³;
R² is hydrogen, halogen, —CN, optionally substituted C₁₋₆ aliphatic, or optionally substituted C₃₋₆ cycloaliphatic;
each R³ is independently hydrogen, halogen, optionally substituted C₁₋₆ aliphatic, or optionally substituted C₃₋₆ cycloaliphatic;
R⁴ is hydrogen, optionally substituted C₁₋₆ aliphatic, or optionally substituted C₃₋₆ cycloaliphatic;
R⁵ is hydrogen, halogen, optionally substituted C₁₋₆ aliphatic, or optionally substituted C₃₋₆ cycloaliphatic, or
R² and R⁵, together with the atoms to which they are attached, combine to form an optionally substituted 5- to 6-membered aromatic ring having 0-2 heteroatoms independently selected from N, O, and S;
R⁶ is hydrogen, optionally substituted C₁₋₆ aliphatic, or optionally substituted C₃₋₆ cycloaliphatic;
X is O or NR⁷;
R⁷ is hydrogen or optionally substituted Cie aliphatic, or
R⁴ and R⁷, together with the atoms to which they are attached, combine to form an optionally substituted 5- to 6-membered aromatic ring having 2-3 heteroatoms independently selected from N, O, and S;
each R⁸ is independently hydrogen or optionally substituted C₁₋₆ aliphatic;
R⁹ is hydrogen or optionally substituted C₁₋₆ aliphatic;
linker is a covalent bond or an optionally substituted, bivalent, straight or branched, saturated or unsaturated C₁-C₂₀ hydrocarbon chain, wherein one or more methylene units are optionally and independently replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —SO₂—, —SO₂N(R)—, —N(R)SO₂—, or -Cy-;
each Cy is independently an optionally substituted, mono- or multicyclic, 3- to 16-membered bivalent ring system, wherein the ring system is fully saturated, partially saturated, or aromatic, and the ring system contains 0-6 heteroatoms independently selected from N, O, and S;
each R is independently hydrogen or an optionally substituted group selected from C₁₋₆ aliphatic, phenyl, C₃₋₇ cycloaliphatic, 5- to 6-membered monocyclic heteroaryl having 1-4 heteroatoms independently selected from N, O, and S, and a 3- to 7-membered monocyclic heterocyclyl having 1-2 heteroatoms independently selected from N, O, and S; and
LBM is an E3 ubiquitin ligase binding moiety.
674. A compound of Formula I:

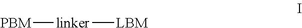

wherein:
PBM is a KAT2 protein binding moiety;
linker is an optional linking moiety; and
LBM is an E3 ubiquitin ligase binding moiety.
675. The compound of Embodiment 674, wherein the PBM is a KAT2A protein binding moiety.
676 The compound of Embodiment 674, wherein the PBM is a KAT2B protein binding moiety.
677. The compound of any one of Embodiments 674-676, wherein the linker is less than 14 atoms in length.
678. The compound of any one of Embodiments 674-677, wherein the linker is less than 11 atoms in length.
679. The compound of any one of Embodiments 674-678, wherein the LBM is a CRBN binding moiety.
680. The compound of any one of Embodiments 674-678, wherein the LBM is a VHL binding moiety.
681. The compound of any one of Embodiments 674-678, wherein the LBM is an IAP binding moiety.
682. The compound of any one of Embodiments 674-678, wherein the LBM is a MDM2 binding moiety.
683. A pharmaceutical composition comprising a compound of any one of Embodiments 597-682, or a pharmaceutically acceptable salt thereof, and a pharmaceutically acceptable carrier.
684. A method of preparing the pharmaceutical composition of Embodiment 683, comprising:
   providing a compound of any one of Embodiments 597-682, or a pharmaceutically acceptable salt thereof; and
   formulating the compound with suitable excipients to give the pharmaceutical composition.
685. A method, comprising administering a compound of any one of Embodiments 597-682, or a pharmaceutically acceptable salt thereof, or a pharmaceutical composition of Embodiment 685 to a subject in need thereof.
686. A method of treating a cancer, comprising administering a compound of any one of Embodiments 597-682, or a pharmaceutically acceptable salt thereof, or a pharmaceutical composition of Embodiment 683 to a subject in need thereof.
687. The method of Embodiment 686, wherein the cancer is selected from acute myeloid leukemia, neuroblastoma, non-small cell lung cancer, small cell lung cancer, colorectal cancer, melanoma, and prostate cancer.
688. A method of degrading KAT2 in a subject, comprising administering a compound of any one of Embodiments 597-682, or a pharmaceutically acceptable salt thereof, or a pharmaceutical composition of Embodiment 683 to a subject in need thereof.
689. An in vitro method of degrading KAT2, comprising contacting a biological sample with a compound of any one of Embodiments 597-682, or a pharmaceutically acceptable salt thereof.

### EXAMPLES

As described in the Examples below, in certain exemplary embodiments, compounds are prepared according to the following general procedures. It will be appreciated that, although the general methods depict the synthesis of certain compounds of the present disclosure, the following general methods and other methods known to one of ordinary skill in the art can be applied to all compounds and subclasses and species of each of these compounds, as described herein. Certain useful technologies including synthetic methods, reagents, intermediates, conditions and characterization technologies are described in WO2024/050078, the entirety of which is incorporated herein by reference, and can be utilized in accordance with the present disclosure.

List of Abbreviations

| | |
|---|---|
| MeOH | Methanol |
| Boc20 | Di-tert-butyl dicarbonate |
| TBDPS-Cl | tert-Butyldiphenylchlorosilane |
| Mel | Methyl iodide |
| DMF | N,N-Dimethylformamide |
| LAH | Lithium aluminum hydride |
| THF | Tetrahydrofuran |
| TsCI | 4-Toluenesulfonyl chloride |
| TEA | Triethylamine |
| DCM | Dichloromethane |
| TFA | Trifluoroacetic acid |
| TCFH | N,N.N',N'-Tetramethylchloroformamidinium |
| | hexafluorophosphate |
| NMI | N-methylimidazole |
| NaOAc | Sodium acetate |
| AcOH | Acetic acid |
| DMA | N,N-Dimethylacetamide |
| EtOAc | Ethyl acetate |
| n-BuLi | n-Butyllithium |
| HMPA | Hexamethylphosphoramide |
| TBAF | Tetrabutylammonium fluoride |
| MsCI | Methanesulfonyl chloride |
| TPP or PPh₃ | Triphenylphosphine |
| DIPEA | N,N-Diisopropylethylamine |
| DMSO | Dimethylsulfoxide |
| EDC.HCI | 1-Ethyl-3-(3-dimethyllaminopropyl) |
| | carbodiimide hydrochloride |
| DMAP | 4-Dimethylaminopyridine |
| HOBt | N-Hydroxybenzotriazole |
| DIAD | Diisopropylazodicarboxylate |
| Pd(PPh₃)₂Cl₂ | trans-Dichlorobis(triphenylphosphine)palladium (II) |
| NaCNBH: | Sodium cyanoborohydride |
| DIPC | N,N'-Diisopropylcarbodiimide |
| ACN | Acetonitrile |
| RT | Room temperature or retention time |
| HPLC | High Performance Liquid Chromatography |
| C-HPLC | Chiral high Performance Liquid Chromatography |
| SOR | Specific Optical Rotation |
| h | Hour |
| min | Minute |
| sat. | Saturated |
| aq. | Aqueous |
| TLC | thin layer chromatography |
| NMR | Nuclear magnetic resonance |
| S | Singlet |
| brs | Broad singlet |
| d | Doublet |
| t | Triplet |
| m | Multiplet |
| dd | Doublet of doublets |
| br | Broad |
| LC-MS | Liquid chromatography and mass spectrometry |
| m/z | Mass to charge ratio |
| prep | Preparative |

### Analytical Methods

The following analytical methods are exemplary of those used to purify and/or characterize the compounds described herein:

Method A (prep-HPLC): Column: X-SELECT CSH-C18, (250×30 mm) 5 μm, Eluents A: 0.1% TFA in Water, B: Acetonitrile; Flow Rate: 25.0 mL/min; Gradient: 0.0 min 5% B, 0.2-3.0 min 5-5% B, 3-40 min 60% B, 40-41 min 60-95% B, 41-47 min 95-95% B, 47-48 min 95-5% B, 48-55 min 5-5% B.

Method B (prep-HPLC): Column: X-SELECT CSH-C18, (250×30 mm) 5 μm, Eluents A: 10 mM Ammonium Bicarbonate in Water, B: Acetonitrile; Flow Rate: 25.0 mL/min; Gradient: 0.0 min 5% B, 0.2-3.0 min 5-5% B, 3-40 min 60% B, 40-41 min 60-95% B, 41-47 min 95-95% B, 47-48 min 95-5% B, 48-55 min 5-5% B.

Method C (prep-HPLC): Column: X-SELECT CSH-C18, (250×30 mm) 5 μm, Eluents A: 0.1% TFA in Water, B: Acetonitrile; Flow Rate: 25.0 mL/min; Gradient: 0.0 min 10% B, 0.2-3.0 min 10-10% B, 3-40 min 10-60% B, 40-41 min 60-95% B, 41-47 min 95-95% B, 47-48 min 95-10% B, 48-55 min 10-10% B.

Method D (Combiflash®): Column: YMC, Dimension: 4 g (irregular silica, 40-63 μm, 60 Å), Eluents A: DCM, B: MeOH, Gradient: 0-5% MeOH/DCM for 30 min, Flow Rate: 20.0 mL/min.

Method E (prep-HPLC): Column: X-SELECT CSH-C18, (250×30 mm) 5 μm, Eluents A: 10 mM Ammonium Bicarbonate in Water. B: Acetonitrile; Flow Rate: 25.0 mL/min; Gradient: Linear gradient.

Method F (prep-HPLC): Column: X-SELECT CSH-C18, (250×30 mm) 5 μm, Eluents A: 0.1% Formic Acid in Water, B: Acetonitrile; Flow Rate: 25.0 mL/min; Gradient: 0.0 min 2% B, 0.2-3.0 min 2-2% B, 3-50 min 2-55% B, 50-51 min 55-95% B, 51-57 min 95-95% B, 57-58 min 95-2% B, 58-65 min 2-2% B.

Method G: Column: X-SELECT CSH-C18, (250×30 mm) 5 μm, Eluents A: 0.1% Formic Acid in Water, B: Acetonitrile; Flow Rate: 25.0 mL/min; Gradient: Linear gradient.

Method H: Column: SYNERGY C18, (150×19 mm) 5 μm, Eluents A: 10 mM ammonium bicarbonate in H₂O, B: Acetonitrile; Flow Rate: 25.0 mL/min; Gradient (Time/% B): 0/10, 10/30, 20/40, 30/50, 40/60, 50/70, 55/98.

### Preparation of Intermediates

### Preparation of Intermediate 1:4-((3R,5R)-5-((5-bromo-1-methyl-6-oxo-1,6-dihydropyridazin-4-yl)amino)-1-methylpiperidin-3-yl)benzoic acid

### Step-1: methyl 4-(5-((tert-butoxycarbonyl)amino)pyridin-3-yl)benzoate

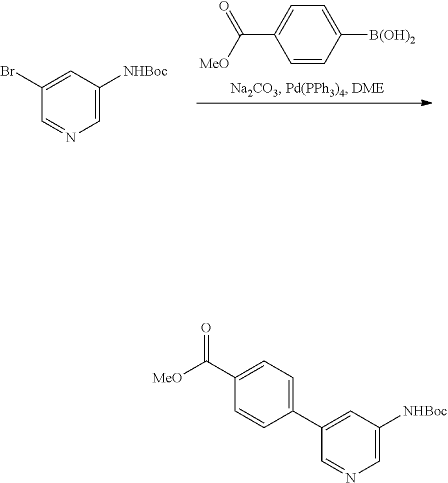 [SMILES: COC(=O)c1ccc(B(O)O)cc1 ; CC(C)(C)OC(=O)Nc1cncc(Br)c1 ; COC(=O)c1ccc(-c2cncc(NC(=O)OC(C)(C)C)c2)cc1]

To a stirred solution of 4-(methoxycarbonyl)phenyl) boronic acid (7.25 g, 40.29 mmol) and tert-butyl (5-bromopyridin-3-yl)carbamate (10.00 g, 36.63 mmol) in DME (150 mL), a solution of Na₂CO₃ (18.60 g, 175.80 mmol) in water (3 mL) was added, and the reaction mixture was purged with argon for 30 min. To the resulting reaction mixture, Pd(PPh₃)₄ (4.23 g, 3.66 mmol) was added, and the reaction mixture was again purged with argon for 20 min. The reaction mixture was stirred at 90° C. for 16 h. The progress of the reaction was monitored by TLC. After completion of reaction, the reaction mixture was filtered through Celite®. The filtrate was diluted with cold water and extracted with ethyl acetate. Combined organic layer was washed with water and brine, dried over anhydrous Na₂SO₄ and concentrated under reduced pressure. The crude compound was purified by silica gel column chromatography (10% ethyl acetate in hexane) to afford methyl 4-(5-((tert-butoxycarbonyl)amino)pyridin-3-yl)benzoate (7.00 g, 58%) as an off white solid. ¹H NMR (400 MHz, DMSO-dc) δ=9.77-9.69 (m, 1H), 8.63 (brs, 1H), 8.55 (s, 1H), 8.25 (brs, 1H), 8.08 (d, J=7.8 Hz, 2H), 7.82 (d, J=8.3 Hz, 2H), 3.89 (s, 3H), 1.50 (s, 9H). LC-MS: m/z 329.2 [M+H]⁺.

### Step-2: 3-((tert-butoxycarbonyl)amino)-5-(4-(methoxycarbonyl)phenyl)-1-methylpyridin-1-ium

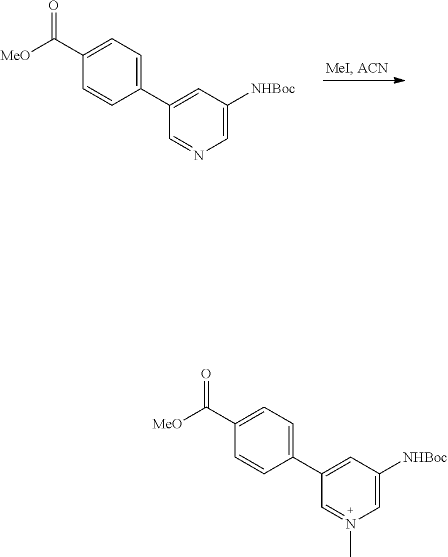

To a stirred solution of methyl 4-(5-((tert-butoxycarbonyl)amino)pyridin-3-yl)benzoate (7.50 g, 22.80 mmol, from step 1) in ACN (70 mL), MeI (2.10 mL, 34.20 mmol) was added at RT. The reaction mixture was stirred at 90° C. for 16 h. The progress of the reaction was monitored by TLC. After completion of reaction, the reaction mixture was quenched with saturated NH₄Cl solution. The aqueous layer was extracted with ethyl acetate. Combined organic layer was washed with brine, dried over anhydrous Na₂SO₄ and concentrated under reduced pressure to give crude compound. The crude compound was washed with diethyl ether to afford 3-((tert-butoxycarbonyl)amino)-5-(4-(methoxycarbonyl)phenyl)-1-methylpyridin-1-ium (6.50 g, 83%) as an off white solid. ¹H NMR (400 MHz, DMSO-d₆) δ=10.57 (brs, 1H), 9.12 (d, J=13.2 Hz, 2H), 8.64-8.52 (m, 1H), 8.19 (d, J=8.3 Hz, 2H), 7.93 (d, J=8.3 Hz, 2H), 4.42 (s, 3H), 3.91 (s, 3H), 1.53 (s, 9H). LC-MS: m/z 344.2 [M+H]⁺.

### Step-3: methyl 4-((cis)-5-((tert-butoxycarbonyl)amino)-1-methylpiperidin-3-yl)benzoate

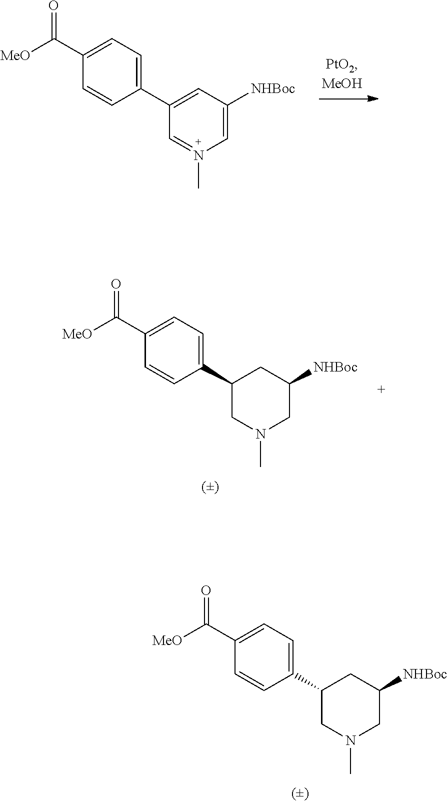

To a stirred solution of 3-((tert-butoxycarbonyl)amino)-5-(4-(methoxycarbonyl)phenyl)-1-methylpyridin-1-ium (2.50 g, 7.28 mmol, from step 2) in methanol (50 mL), PtO₂ (1.25 g) was added at RT under hydrogen atmosphere. The reaction mixture was stirred at RT for 16 h. Progress of reaction was monitored by TLC and LCMS. After completion of reaction, the reaction mixture was filtered through Celite®. The filtrate was concentrated to give crude compound. The crude compound was purified by column chromatography (5% methanol in DCM) to afford compound methyl 4-((cis)-5-((tert-butoxycarbonyl)amino)-1-methylpiperidin-3-yl)benzoate (1.70 g, 68%) as an off-white solid along with compound methyl 4-((trans)-5-((tert-butoxycarbonyl)amino)-1-methylpiperidin-3-yl)benzoate. LC-MS: m/z 349.3 [M+H]⁺.

### Step-4: methyl 4-((cis)-5-amino-1-methylpiperidin-3-yl)benzoate

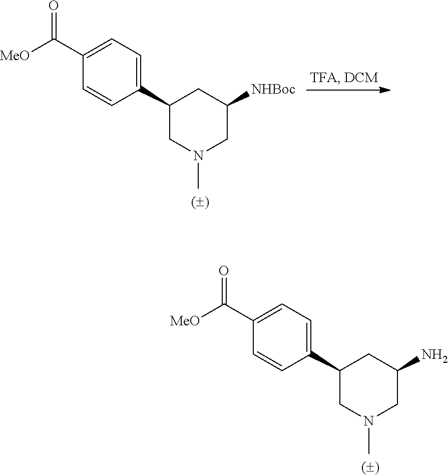 [SMILES: COC(=O)c1ccc([C@H]2C[C@@H](NC(=O)OC(C)(C)C)CN(C)C2)cc1 ; COC(=O)c1ccc([C@H]2C[C@@H](N)CN(C)C2)cc1]

To a stirred solution of methyl 4-((cis)-5-((tert-butoxycarbonyl)amino)-1-methylpiperidin-3-yl)benzoate (3.40 g, 7.28 mmol, from step 3) in DCM (30 mL), TFA (10 mL) was added slowly at 0° C. and the reaction mixture was stirred at RT for 16 h. Progress of reaction was monitored by TLC and LCMS. The reaction mixture was concentrated under reduced pressure and co-distilled with DCM and methanol. The residue was diluted with saturated NaHCO₃ solution and extracted with DCM. Combined organic layer was dried over anhydrous Na₂SO₄ and concentrated to afford crude methyl 4-((cis)-5-amino-1-methylpiperidin-3-yl)benzoate (2.00 g, 83%) as a yellow sticky solid which was used as such for the next reaction. ¹H NMR (400 MHz, DMSO-d₆) δ=7.89 (d, J=7.8 Hz, 2H), 7.39 (d, J=7.8 Hz, 2H), 3.83 (s, 3H), 2.91-2.70 (m, 4H), 2.18 (s, 3H), 1.91 (d, J=11.2 Hz, 1H), 1.79 (t, J=11.0 Hz, 1H), 1.54 (t, J=10.0 Hz, 1H), 1.21-1.09 (m, 1H), 2H merged in solvent peak. LC-MS: m/z 249.4 [M+H]⁺.

### Step-5 and 6: methyl 4-((3R,5R)-5-((5-bromo-1-methyl-6-oxo-1,6-dihydropyridazin-4-yl)amino)-1-methylpiperidin-3-yl)benzoate

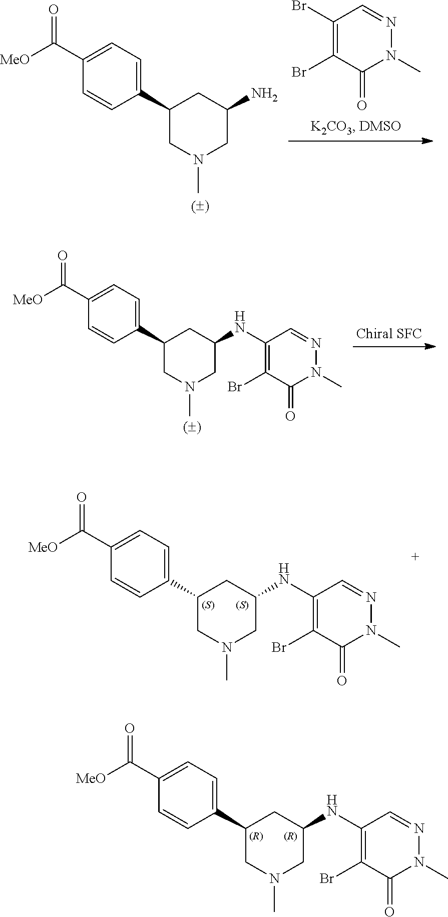

To a stirred solution of methyl 4-((cis)-5-amino-1-methylpiperidin-3-yl)benzoate (3.00 g, 12.09 mmol, from step 4) in DMSO (30 mL), K₂CO₃ (5.00 g, 36.29 mmol) was added, followed by 4,5-dibromo-2-methylpyridazin-3 (2H)-one (intermediate 2, 4.86 g, 18.14 mmol), and the reaction mixture was stirred at 100° C. for 16 h. Progress of reaction was monitored by TLC and LCMS. After completion of reaction, the reaction mixture was dissolved in water and extracted with ethyl acetate. The combined organic layer was dried over anhydrous Na₂SO₄ and concentrated under reduced pressure. The crude compound was purified by column chromatography (2% methanol in DCM) to afford methyl 4-((cis)-5-((5-bromo-1-methyl-6-oxo-1,6-dihydropyridazin-4-yl)amino)-1-methylpiperidin-3-yl)benzoate (0.84 g, 16%) as pale-yellow solid. This compound was purified by chiral SFC to afford methyl 4-((3S,5S)-5-((5-bromo-1-methyl-6-oxo-1,6-dihydropyridazin-4-yl)amino)-1-methylpiperidin-3-yl)benzoate and methyl 4-((3R,5R)-5-((5-bromo-1-methyl-6-oxo-1,6-dihydropyridazin-4-yl)amino)-1-methylpiperidin-3-yl)benzoate (0.20 g, 24%). The chiral SFC conditions were as follows: Phenomenex Cellulose-3 column (250 mm×21.2 mm, 5 μm); mobile phase: methanol; flow rate: 18 mL/min; diluent: methanol; loading: 80 mg/injection. Isomer 1 at RT 10.5 min and Isomer 2 at 18 min.

Isomer 1: methyl 4-((3S,5S)-5-((5-bromo-1-methyl-6-oxo-1,6-dihydropyridazin-4-yl)amino)-1-methylpiperidin-3-yl)benzoate: ¹H NMR (400 MHz, DMSO-d₆) δ=7.96-7.85 (m, 3H), 7.42 (d, J=7.8 Hz, 2H), 5.82 (d, J=9.3 Hz, 1H), 4.03-3.91 (m, 1H), 3.84 (s, 3H), 3.59 (s, 3H), 3.05 (t, J=11.5 Hz, 1H), 2.95 (d, J=7.3 Hz, 1H), 2.85 (d, J=10.8 Hz, 1H), 2.25 (s, 3H), 2.07-1.87 (m, 3H), 1.72 (q, J=11.7 Hz, 1H). LC-MS: m/z 435.0 [M+H]⁺. C-HPLC (Column: Chiralcel OJ-H (250*4.5 mm, 5 μm); Eluents A: CO₂, B: 0.1% DEA in EtOH; A:B-40% B Isocratic): RT=2.50 mins; 100.00%. SOR: −19.16, Solvent: MeOH, Path length: 50 mm, Concentration: 0.1 w/v %.

Isomer 2: methyl 4-((3R,5R)-5-((5-bromo-1-methyl-6-oxo-1,6-dihydropyridazin-4-yl)amino)-1-methylpiperidin-3-yl)benzoate: ¹H NMR (400 MHz, DMSO-d₆) δ=7.95-7.85 (m, 3H), 7.42 (d, J=8.3 Hz, 2H), 5.82 (d, J=9.3 Hz, 1H), 4.02-3.89 (m, 1H), 3.84 (s, 3H), 3.59 (s, 3H), 3.05 (t, J=11.5 Hz, 1H), 2.95 (d, J=6.8 Hz, 1H), 2.90-2.80 (m, 1H), 2.25 (s, 3H), 2.09-1.87 (m, 3H), 1.80-1.66 (m, 1H). LC-MS: m/z 435.1 [M+H]⁺. C-HPLC (Column: Chiralcel OJ-H (250*4.5 mm, 5 μm); Eluents A: CO₂, B: 0.1% DEA in EtOH; A:B-40% B Isocratic): RT=3.26 mins: 99.17%. SOR: 21.04, Solvent: MeOH, Path length: 50 mm, Concentration: 0.1 w/v %.

### Step-7: 4-((3R,5R)-5-((5-bromo-1-methyl-6-oxo-1,6-dihydropyridazin-4-yl)amino)-1-methylpiperidin-3-yl)benzoic acid

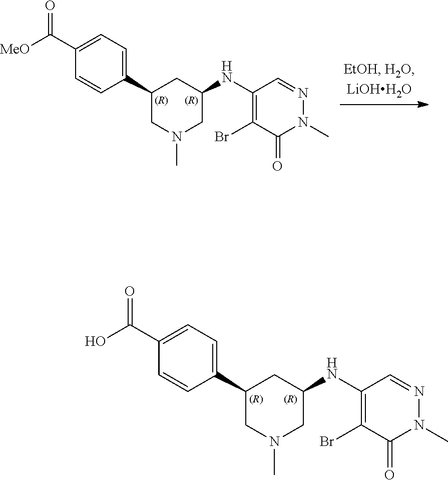

To a stirred solution of methyl 4-((3R,5R)-5-((5-bromo-1-methyl-6-oxo-1,6-dihydropyridazin-4-yl)amino)-1-methylpiperidin-3-yl)benzoate (0.23 g, 0.52 mmol, from step 6) in ethanol (5 mL) and water (0.5 mL), lithium hydroxide (0.03 g, 0.63 mmol) was added and the reaction mixture was stirred at 70° C. for 2 h. The progress of the reaction was monitored by TLC. The reaction mixture was concentrated under reduced pressure. The crude was acidified with 1N HCl to pH 3. Precipitated solid was filtered, washed and concentrated to yield 4-((3R,5R)-5-((5-bromo-1-methyl-6-oxo-1,6-dihydropyridazin-4-yl)amino)-1-methylpiperidin-3-yl)benzoic acid (0.20 g, 91%) as an off white solid. ¹H NMR (400 MHz, DMSO-d₆) δ=13.13-12.82 (m, 1H), 11.34-11.09 (m, 1H), 7.96 (d, J=6.8 Hz, 3H), 7.43 (d, J=8.3 Hz, 2H), 6.29 (d, J=8.3 Hz, 1H), 4.53-4.36 (m, 1H), 3.62 (s, 3H), 3.57-3.47 (m, 1H), 3.46-3.36 (m, 1H), 3.15-3.03 (m, 1H), 2.93 (d, J=8.8 Hz, 1H), 2.80 (s, 3H), 2.18 (d, J=10.3 Hz, 1H), 2.12-2.01 (m, 1H). LC-MS: m/z 422.0 [M+H]⁺.

### Alternate Preparation of Intermediate 1: 4-((3R,5R)-5-((5-bromo-1-methyl-6-oxo-1,6-dihydropyridazin-4-yl)amino)-1-methylpiperidin-3-yl)benzoic acid

### Step 1: tert-butyl (3R)-3-(pyridine-2-carbonylamino)piperidine-1-carboxylate

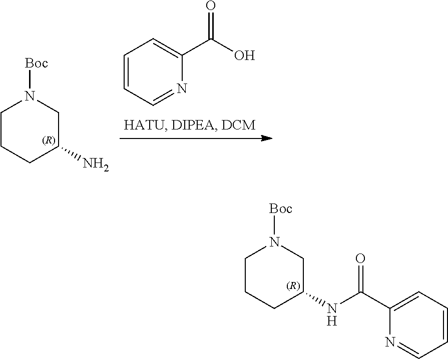

To a stirred solution of picolinic acid (10 g, 50.00 mmol), tert-butyl (R)-3-aminopiperidine-1-carboxylate (7.40 g, 60.00 mmol) and DIPEA (13 mL, 75.00 mmol) in DCM (100 mL) was added HATU (23 g, 60.00 mmol) and the reaction mixture was stirred at ambient temperature for 16 h. The progress of the reaction was monitored by TLC. After completion of reaction, the reaction mixture was concentrated under reduced pressure. The residue was dissolved in ethyl acetate and washed with 0.05 M HCl and saturated NaHCO₃ solution. The organic layer was dried over anhydrous Na₂SO₄ and concentrated under reduced pressure. The crude compound was purified by column chromatography to afford tert-butyl (3R)-3-(pyridine-2-carbonylamino)piperidine-1-carboxylate (13.17 g, 72%) as a white solid. ¹H NMR (400 MHz, DMSO-d₆) δ=8.63 (d, J=3.9 Hz, 1H), 8.55 (brs, 1H), 8.07-7.98 (m, 2H), 7.61 (t, J=5.6 Hz, 1H), 3.90-3.79 (m, 2H), 3.76-3.49 (m, 2H), 2.99 (brs, 2H), 1.82 (brs, 1H), 1.74-1.61 (m, 2H), 1.37 (brs, 9H); LC-MS: m/z 306.2 [M+H]⁺.

### Step 2: tert-butyl (3R,5R)-3-(4-methoxycarbonylphenyl)-5-(pyridine-2-carbonylamino)piperidine-1-carboxylate

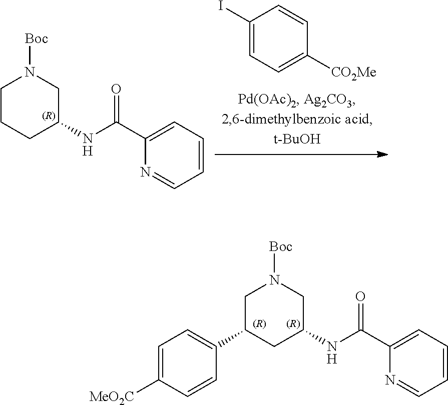

A pressure vial (100 mL) equipped with a magnetic stirring bar was charged with tert-butyl (3R)-3-(pyridine-2-carbonylamino)piperidine-1-carboxylate (10.00 g, 32.8 mmol, from step 1), silver carbonate (9 g, 32.8 mmol). Pd(OAc)₂ (0.74 g, 0.32 mmol), methyl 4-iodobenzoate (43 g, 164 mmol), 2,6-dimethylbenzoic acid (1.23 g, 8.2 mmol) and t-BuOH (100 mL). The vessel was flushed with argon, sealed with a crimp cap and heated to 120° C. After 24 h, the reaction vessel was removed from the oil bath and cooled to room temperature and DCM was added to the reaction mixture. The progress of the reaction was monitored by TLC. The mixture was thoroughly stirred for 10 min and the solids were removed by filtration which was additionally rinsed with DCM. The combined filtrates were concentrated under reduced pressure and the residue was purified by column chromatography to afford tert-butyl (3R,5R)-3-(4-methoxycarbonylphenyl)-5-(pyridine-2-carbonylamino)piperidine-1-carboxylate (8.00 g, 56%) as a white solid. LC-MS: m/z 438.12 [M−H]⁻.

### Step 3: Methyl 4-[(3R,5R)-5-(pyridine-2-carbonylamino)-3-piperidyl]benzoate

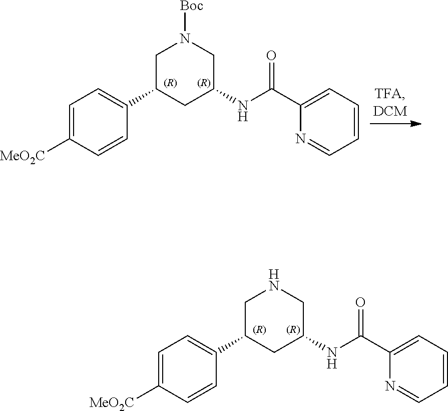

To a stirring solution of tert-butyl (3R,5R)-3-(4-methoxycarbonylphenyl)-5-(pyridine-2-carbonylamino)piperidine-1-carboxylate (6.00 g, 13.66 mmol, from step 2) in DCM (70 mL) was added TFA (12 mL) at 0° C. Then the resulting solution was slowly brought to room temperature and stirred for 7 h. The progress of the reaction was monitored by TLC. The solvent was removed under reduced pressure, and the crude product was redissolved in water and extracted with ethyl acetate. The combined organic layer was dried over anhydrous Na₂SO₄, filtered, and concentrated in vacuo to afford methyl 4-[(3R,5R)-5-(pyridine-2-carbonylamino)-3-piperidyl]benzoate (2.7 g, 58%) as a pale-yellow solid which was used for the next reaction without any further purification. ¹H NMR (400 MHz, DMSO-d₆) δ=8.65-8.63 (m, 1H), 8.52 (d, J=8.6 Hz, 1H), 8.05-7.97 (m, 2H), 7.92-7.89 (m, 2H), 7.62-7.58 (m, 1H), 7.41 (d, J=8.3 Hz, 2H), 4.06-4.00 (m, 1H), 3.84 (s, 3H), 3.08-2.95 (m, 3H), 2.91-2.85 (m, 1H), 2.55 (s, 1H), 2.45 (s, 1H), 2.04 (d, J=12.2 Hz, 1H), 1.93-1.85 (m, 1H); LC-MS: m/z 340.22 [M+H]⁺.

### Step 4: methyl 4-[(3R,5R)-1-methyl-5-(pyridine-2-carbonylamino)-3-piperidyl]benzoate

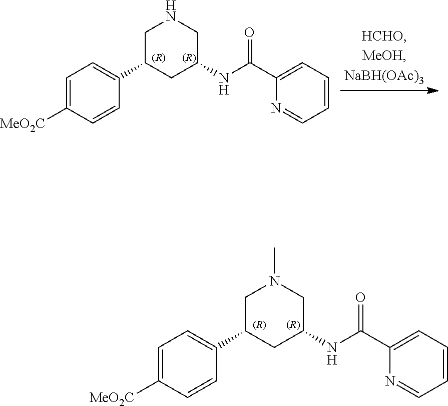

To a stirred solution of methyl 4-[(3R,5R)-5-(pyridine-2-carbonylamino)-3-piperidyl]benzoate (7.0 g, 21 mmol, from step 3) and glacial acetic acid (1 mL) in methanol (70 mL) was added 37% w/v formaldehyde (2.9 mL) and stirred for 5 h. Then sodium triacetoxyborohydride (6.53 g, 31 mmol) was added over a period of 30 min at 0° C. The resulting solution was stirred for 16 h at RT. The progress of the reaction was monitored by TLC. After completion of reaction, the solvent was removed in vacuo and the residue was partitioned between ethyl acetate and water. The biphasic solution was extracted twice with ethyl acetate. The combined organic layer was washed with brine, dried over anhydrous Na₂SO₄, filtered and concentrated. Crude material was purified by CombiFlash® column chromatography to afford methyl 4-[(3R,5R)-1-methyl-5-(pyridine-2-carbonylamino)-3-piperidyl]benzoate (5.5 g, 75%). ¹H NMR (400 MHz, DMSO-d=) δ=8.67-8.53 (m, 2H), 8.08-7.95 (m, 2H), 7.91 (d, J=8.3 Hz, 2H), 7.63-7.55 (m, 1H), 7.44 (d, J=8.3 Hz, 2H), 4.21-4.06 (m, 1H), 3.84 (s, 3H), 3.09-2.90 (m, 2H), 2.85 (d, J=9.8 Hz, 1H), 2.25 (s, 3H), 2.04-1.89 (m, 3H), 1.84-1.69 (m, 1H); LC-MS: m/z 354.12 [M+H]⁺.

### Step-5: methyl 4-((3R,5R)-5-amino-1-methylpiperidin-3-yl)benzoate

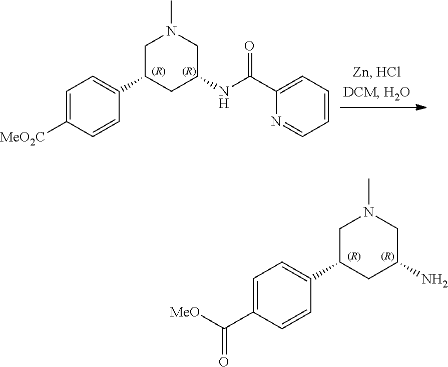

To a stirred suspension of methyl 4-[(3R,5R)-1-methyl-5-(pyridine-2-carbonylamino)-3-piperidyl]benzoate (1.00 g, 2.80 mmol, from step 4) in water (50 mL) was added 12 N HCl solution (5 mL) at 0° C., followed by zinc powder (2.76 g, 42.00 mmol) and DCM (50 mL) the reaction mixture was stirred at rt for 12 h in a round bottomed flask. The progress of the reaction was monitored by TLC. After completion of the reaction, cooled to 0° C. and basified using NaOH (5M) until pH 9. Then the mixture was filtered through Celite® bed and washed with DCM (20 mL×3). The filtrate was extracted with DCM (20 mL×3), all organic layers were combined, and solvent was removed in vacuo. Crude material (700 mg) was purified by CombiFlash® column chromatography (DCM:MeOH:NH₄OH 80:20:03) to afford methyl 4-((3R,5R)-5-amino-1-methylpiperidin-3-yl)benzoate (0.45 g, 64%) as an off white solid. LC-MS: m/z 249.37 [M+H]⁺.

### Step-6: 4-((3R,5R)-5-((5-bromo-1-methyl-6-oxo-1,6-dihydropyridazin-4-yl)amino)-1-methylpiperidin-3-yl)benzoic acid

4-((3R,5R)-5-((5-bromo-1-methyl-6-oxo-1,6-dihydropyridazin-4-yl)amino)-1-methylpiperidin-3-yl)benzoic acid was prepared from methyl 4-((3R,5R)-5-amino-1-methylpiperidin-3-yl)benzoate generally following the procedures in steps 5 and 7 of the Preparation of Intermediate 1 above.

### Preparation of Compounds

### General Procedure 1

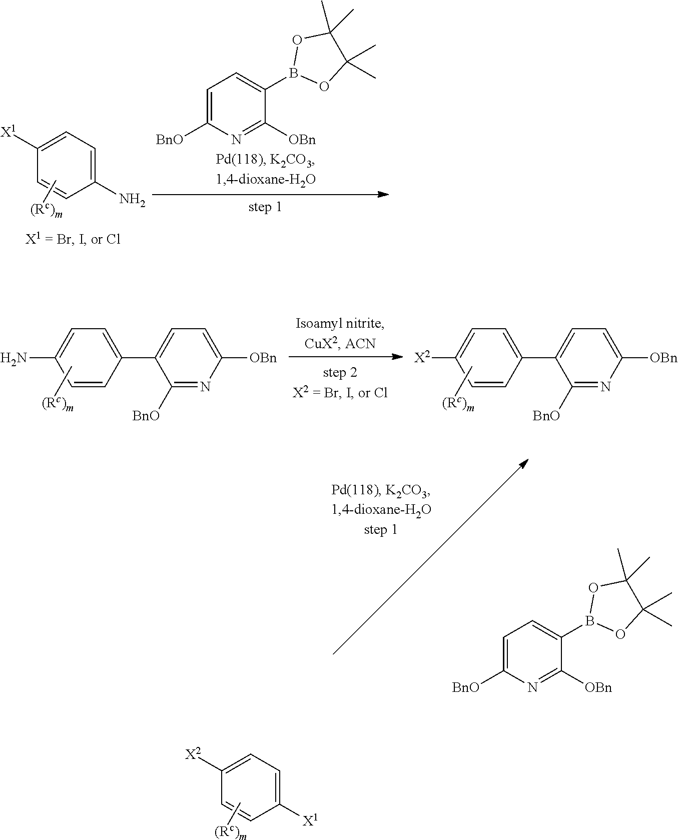

### Step 1

To a stirred solution of an aniline or a dihaloarene (13.00 mmol) in 1,4-dioxane (25 mL) was added 2,6-dibenzyloxy-3-(4,4,5,5-tetramethyl-1,3,2-dioxaborolan-2-yl)pyridine (15.00 mmol) followed by potassium carbonate (25.00 mmol) dissolved in water and the reaction mixture was purged with argon for 30 min. To the resulting reaction mixture, [1,1′-bis(di-tert-butylphosphino)ferrocene]dichloropalladium(II), Pd(118) (1.30 mmol) was added and the reaction mixture was again purged with argon for 20 min. The reaction mixture was stirred in a sealed tube at 100° C. for 16 h. The progress of the reaction was monitored by TLC. After completion of reaction, the reaction mixture was filtered through Celite®. The filtrate was diluted with cold water and extracted with ethyl acetate. Combined organic layer was washed with water and brine, dried over anhydrous Na₂SO₄ and concentrated under reduced pressure. The crude compound was purified by silica gel column chromatography to afford desired product.

### Stop 2

To a stirred solution of intermediate from step 1 (4.80 mmol) in ACN (40 mL) was added isoamyl nitrite (29.00 mmol) at RT and was stirred for 10 min. To this was added copper(I) iodide (14.00 mmol) at RT. The reaction mixture was stirred at 60° C. for 1 h. The progress of reaction was monitored by TLC and LCMS. After completion of reaction, the reaction mixture was diluted with cold water and extracted with ethyl acetate. Combined organic layer was washed with water and brine, dried over anhydrous Na₂SO₄ and concentrated under reduced pressure. The crude compound was purified by silica-gel column chromatography to afford desired product.

### Step 3

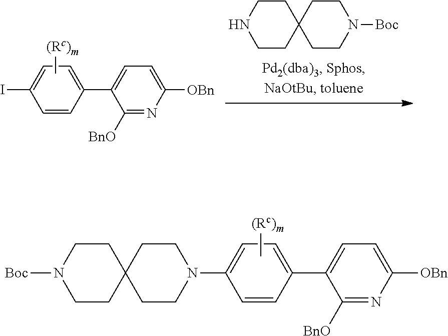

To a stirred solution of intermediate from step 2 (2.30 mmol) in toluene (20 mL) was added tert-butyl 3,9-diazaspiro[5.5]undecane-3-carboxylate (2.70 mmol) followed by sodium tert-butoxide (3.40 mmol) at RT. The reaction mixture was purged with argon for 30 min. To the resulting reaction mixture, 2-dicyclohexylphosphino-2′,6′-dimethoxybiphenyl (0.23 mmol) and tris(dibenzylidencacetone)dipalladium(0) (0.11 mmol) were added at RT and the reaction mixture was again purged with argon for 20 min. The reaction mixture was stirred in a sealed tube at 110° C. for 16 h. The progress of the reaction was monitored by TLC. After completion of reaction, the reaction mixture was filtered through Celite®. The filtrate was diluted with cold water and extracted with ethyl acetate. Combined organic layer was washed with water and brine, dried over anhydrous Na₂SO₄ and concentrated under reduced pressure. The crude compound was purified by silica-gel column chromatography to afford the desired product.

### Step 4

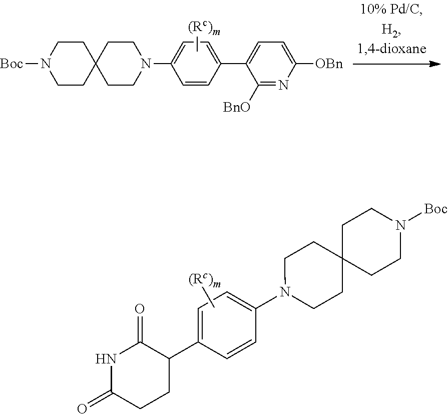

To a stirred solution of intermediate from step 3 (0.69 mmol) in 1,4-dioxane (10 mL) was added 10% Pd/C at RT. The reaction mixture was stirred in a steel bomb under 50 psi H₂ gas pressure at RT for 16 h. The progress of the reaction was monitored by TLC and LCMS. After completion of reaction, the reaction mixture was filtered through Celite®. The filtrate was concentrated under reduced pressure. The crude compound was purified by silica gel column chromatography to afford the desired product.

### Step 5

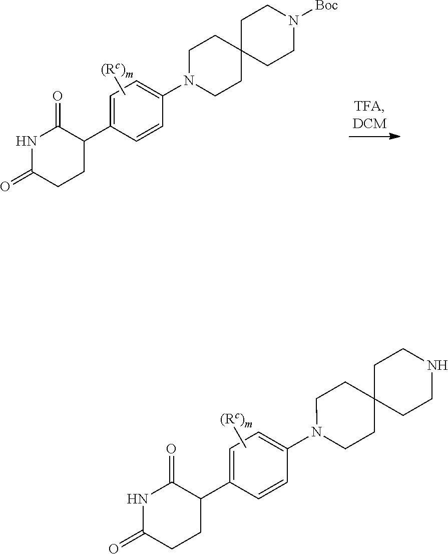

To a stirred solution of the intermediate from step 4 (0.49 mmol) in DCM (5 mL) was added TFA (2 mL) at 0° C. and the reaction mixture was stirred at 0° C. for 5 min followed by at RT for 3 h. The progress of the reaction was monitored by TLC. After completion of reaction, the reaction mixture was concentrated under reduced pressure to afford the desired product.

### Step 6

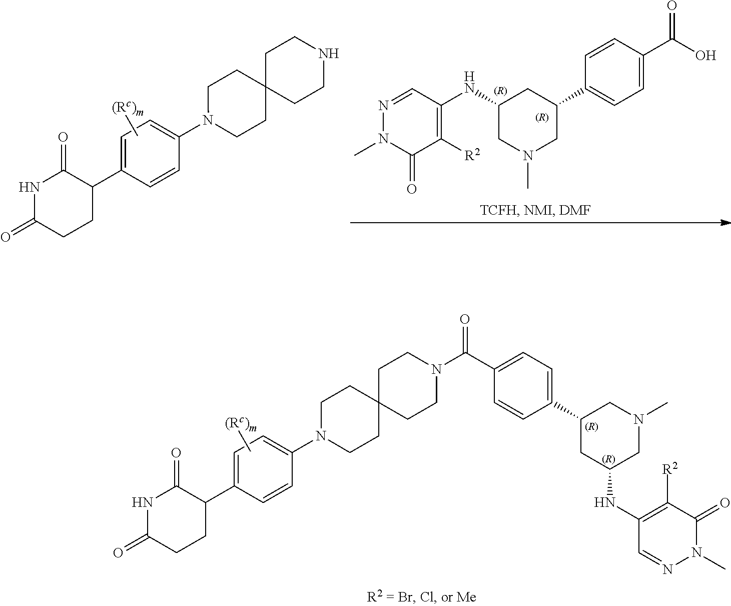

To a stirred solution of the intermediate from step 5 (0.30 mmol) in DMF (4 mL) was added 1-methylimidazole (1.00 mmol) followed by a benzoic acid intermediate (0.30 mmol) and the reaction mixture was stirred at RT for 10 min. To this was added N,N,N′,N′-tetramethylchloroformamidinium hexafluorophosphate (0.40 mmol) and the reaction mixture was stirred at 80° C. for 16 h. The progress of the reaction was monitored by TLC. After completion of reaction, the reaction mixture was diluted with ice-cold water and extracted with ethyl acetate. The combined organic layer was dried over anhydrous Na₂SO₄ and concentrated under reduced pressure, the crude compound was purified by prep HPLC to give the desired product.

### General Procedure 2

### Step 1

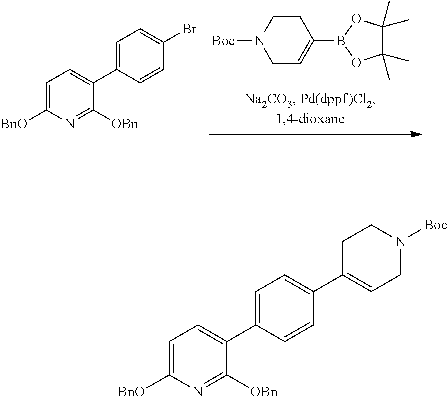 [SMILES: Brc1ccc(-c2ccc(OCc3ccccc3)nc2OCc2ccccc2)cc1 ; CC(C)(C)OC(=O)N1CC=C(B2OC(C)(C)C(C)(C)O2)CC1 ; CC(C)(C)OC(=O)N1CC=C(c2ccc(-c3ccc(OCc4ccccc4)nc3OCc3ccccc3)cc2)CC1]

To a stirred solution of 2,6-dibenzyloxy-3-(4-bromophenyl)pyridine (0.45 mmol, intermediate 11) and tert-butyl 4-(4,4,5,5-tetramethyl-1,3,2-dioxaborolan-2-yl)-3,6-dihydro-2H-pyridine-1-carboxylate (0.54 mmol) in 1,4-dioxane, Na₂CO₃ (0.67 mmol) was added dissolved in H₂O (1.2 mL) at RT. The reaction mixture was purged with argon for 30 min. To the resulting reaction mixture, PdCl₂(dppf) (0.04 mmol) was added and the reaction mixture was again purged with argon for 20 min. The reaction mixture was stirred in sealed tube at 100° C. for 16 h. The progress of the reaction was monitored by TLC. After completion of reaction, the reaction mixture was filtered through Celite®. The filtrate was diluted with cold water and extracted with ethyl acetate. Combined organic layer was washed with water and brine, dried over anhydrous Na₂SO₄ and concentrated under reduced pressure to afford desired product.

### Step 2

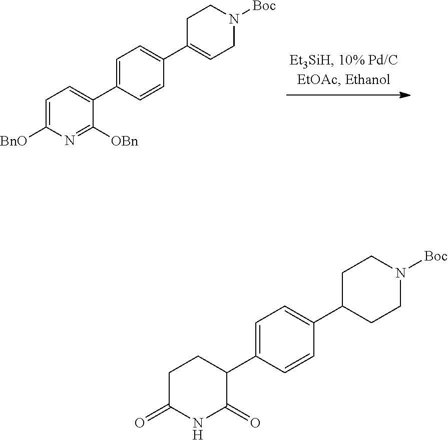 [SMILES: CC(C)(C)OC(=O)N1CC=C(c2ccc(-c3ccc(OCc4ccccc4)nc3OCc3ccccc3)cc2)CC1 ; CC(C)(C)OC(=O)N1CCC(c2ccc(C3CCC(=O)NC3=O)cc2)CC1]

To a stirred solution of tert-butyl 4-[4-(2,6-dibenzyloxy-3-pyridyl)phenyl]-3,6-dihydro-2H-pyridine-1-carboxylate (0.25 mmol, from step 2) in EtOAc (0.5 mL) and EtOH (0.5 mL), 10% Pd/C was added followed by triethyl silane (2.55 mmol) at RT. The reaction mixture was stirred at RT for 16 h. The progress of the reaction was monitored by TLC. After completion of reaction, the reaction mixture was filtered through Celite®. The filtrate was concentrated under reduced pressure to afford desired product.

### Step 3

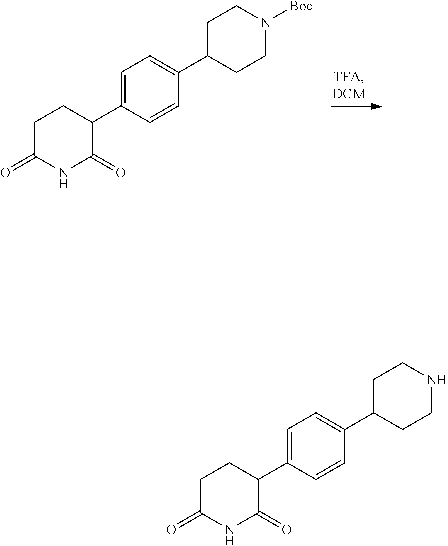 [SMILES: CC(C)(C)OC(=O)N1CCC(c2ccc(C3CCC(=O)NC3=O)cc2)CC1 ; O=C1CCC(c2ccc(C3CCNCC3)cc2)C(=O)N1]

To a stirred solution of tert-butyl 4-[4-(2,6-dioxo-3-piperidyl)phenyl]piperidine-1-carboxylate (0.16 mmol, from step 3) in DCM (2 mL) was added 30% TFA in DCM (4 mL) at 0° C. and the reaction mixture was stirred at 0° C. for 5 min followed by at RT for 4 h. The progress of the reaction was monitored by TLC. After completion of reaction, the reaction mixture was concentrated under reduced pressure to afford desired product.

### Step 4

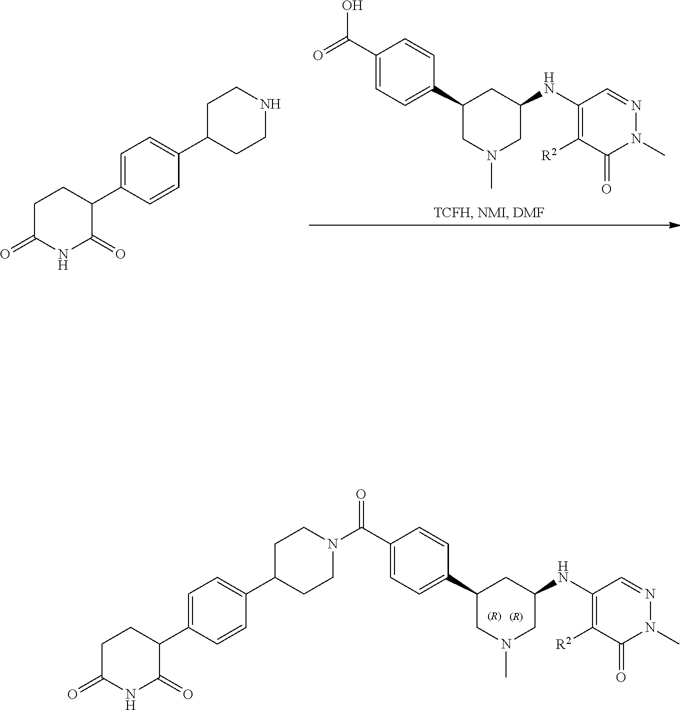

To a stirred solution of 3-[4-(4-piperidyl)phenyl]piperidine-2,6-dione (0.22 mmol, from step 3) in DMF (3 mL), a benzoic acid intermediate (0.22 mmol, intermediate 1, 8, or 17) was added followed by N-methyl imidazole (1.10 mmol) and the reaction mixture was stirred at RT for 10 min. To the resulting reaction mixture, N,N,N′,N′-tetramethylchloroformamidinium hexafluorophosphate (0.33 mmol) was added and stirred at 85° C. for 16 h. The progress of the reaction was monitored by TLC. After completion of reaction, the reaction mixture was diluted with ice-cold water. The precipitated solid was filtered to yield crude compound which was purified by prep HPLC to afford desired product.

### General Procedure 3

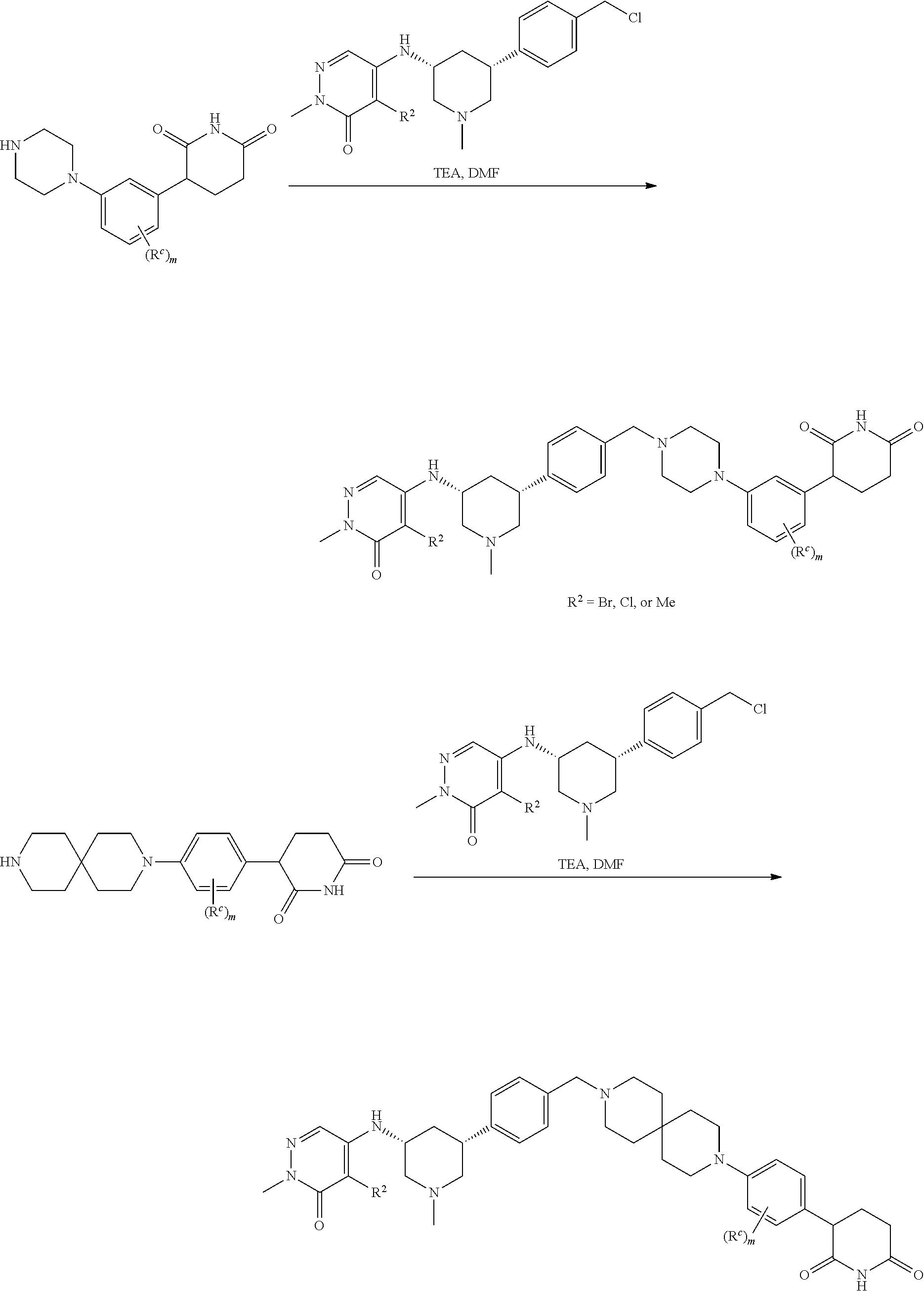

To a stirred solution of an amine intermediate (0.34 mmol) in DMF (1 mL) was added triethylamine (1.00 mmol) at RT. The reaction mixture was stirred at RT for 5 min. To this was added Intermediate 6, 12, or 15 (0.31 mmol) at RT and the reaction mixture was stirred at 100° C. for 16 h. The progress of the reaction was monitored by TLC. After completion of reaction, the reaction mixture was diluted with water and extracted with DCM. The combined organic layer was dried over anhydrous Na₂SO₄ and concentrated under reduced pressure. The crude compound was purified by prep HPLC to afford the desired product.

### Example 1: 3-(5-(4-((1-(4-((3R,5R)-5-((5-bromo-1-methyl-6-oxo-1,6-dihydropyridazin-4-yl)amino)-1-methylpiperidin-3-yl)benzoyl)piperidin-4-yl)methyl)piperazin-1-yl)-1-oxoisoindolin2yl)piperidine-2,6-dione (Compound 22)

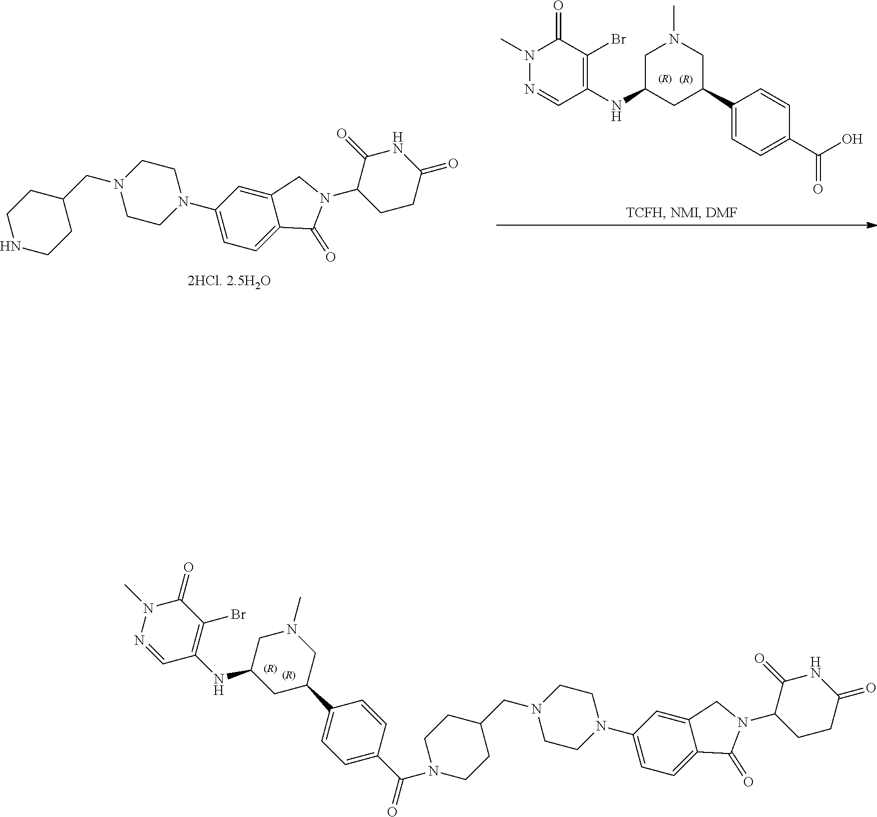

To a stirred solution of 4-((3R,5R)-5-((5-bromo-1-methyl-6-oxo-1,6-dihydropyridazin-4-yl)amino)-1-methylpiperidin-3-yl)benzoic acid (0.02 g, 0.04 mmol, intermediate 1) in DMF (2 mL), 3-(1-oxo-5-(4-(piperidin-4-ylmethyl)piperazin-1-yl) isoindolin-2-yl)piperidine-2,6-dione (0.02 g, 0.04 mmol) was added, followed by N-methylimidazole (0.01 g, 0.20 mmol), and the reaction mixture was stirred at RT for 5 min. To the resulting reaction mixture, N,N,N′,N′-tetramethylchloroformamidinium hexafluorophosphate (0.01 g, 0.06 mmol) was added and stirred at 80° C. for 5 h. The progress of the reaction was monitored by TLC. After completion of reaction, the reaction mixture was diluted with water and extracted with ethyl acetate. The combined organic layer was dried over anhydrous Na₂SO₄ and concentrated under reduced pressure. The crude compound was purified by prep HPLC (Method B) to afford 3-(5-(4-((1-(4-((3R,5R)-5-((5-bromo-1-methyl-6-oxo-1,6-dihydropyridazin-4-yl)amino)-1-methylpiperidin-3-yl)benzoyl)piperidin-4-yl)methyl)piperazin-1-yl)-1-oxoisoindolin-2-yl)piperidine-2,6-dione (0.02 g, 66%) as an off white solid. ¹H NMR (400 MHz, DMSO-d₆) δ=10.93 (s, 1H), 7.87 (s, 1H), 7.53-7.49 (m, 1H), 7.35-7.28 (m, 4H), 7.08-7.01 (m, 2H), 5.80 (d, J=9.0 Hz, 1H), 5.07-5.00 (m, 1H), 4.52-4.39 (m, 1H), 4.36-4.28 (m, 1H), 4.24-4.16 (m, 1H), 4.01-3.91 (m, 1H), 3.59 (s, 3H), 3.29-3.20 (m, 7H), 3.09-2.78 (m, 8H), 2.63-2.55 (m, 1H), 2.40-2.31 (m, 1H), 2.25 (s, 3H), 2.21 (d, J=6.9 Hz, 2H), 2.06-1.89 (m, 4H), 1.89-1.78 (m, 2H), 1.77-1.66 (m, 2H), 1.14-1.01 (m, 2H). LC-MS: m/z 828.4 and 830.2 [M+H]⁺.

### Example 2: 3-[3-[4-[[4-[(3R,5R)-5-[(5-bromo-1-methyl-6-oxo-pyridazin-4-yl)amino]-1-methyl-3-piperidyl]phenyl]methyl]piperazin-1-yl]phenyl]piperidine-2,6-dione (Compound 84)

### Step-1: tert-butyl 4-(3-bromophenyl)piperazine-1-carboxylate

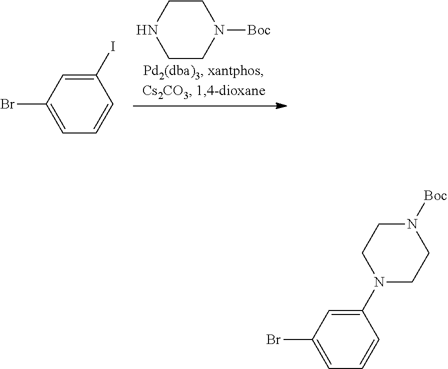

To a stirred solution of 1-bromo-3-iodo-benzene (3.0 g, 10.63 mmol) and tert-butyl piperazine-1-carboxylate (1.96 g, 10.63 mmol), Cs₂CO₃ (10.4 g, 32.0 mmol) was dissolved in 1,4-dioxane (35 mL) at RT. The reaction mixture was purged with argon for 30 min. To the resulting reaction mixture, Pd₂(dba)₃ (0.24 g, 0.26 mmol) and xantphos-Pd (0.42 g, 0.74 mmol) was added and the reaction mixture was again purged with argon for 20 min. The reaction mixture was stirred in a scaled tube at 110° C. for 16 h. The progress of the reaction was monitored by TLC. After completion of reaction, the reaction mixture was filtered through Celite®. The filtrate was diluted with cold water and extracted with ethyl acetate. Combined organic layer was washed with water and brine, dried over anhydrous Na₂SO₄ and concentrated under reduced pressure to afford tert-butyl 4-(3-bromophenyl)piperazine-1-carboxylate (2.34 g. crude) as an off white solid which was used as such for the next reaction. LC-MS: m/z 341.1 [M+H]⁺.

### Step-2: tert-butyl 4-[3-(4,4,5,5-tetramethyl-1,3,2-dioxaborolan-2-yl)phenyl]piperazine-1-carboxylate

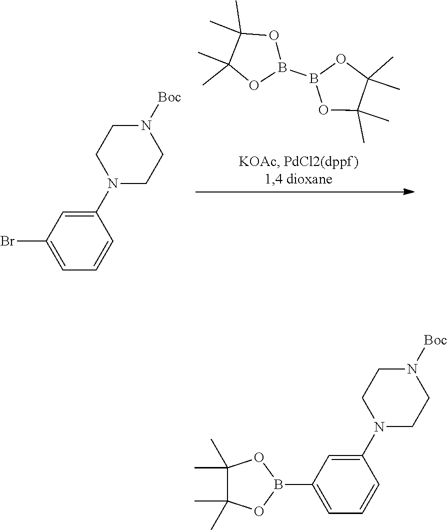

To a stirred solution of tert-butyl 4-(3-bromophenyl)piperazine-1-carboxylate (2.00 g, 5.86 mmol, from step 1) and 4,4,5,5-tetramethyl-2-(4,4,5,5-tetramethyl-1,3,2-dioxaborolan-2-yl)-1,3,2-dioxaborolane (3.00 g, 11.72 mmol) in 1,4-dioxane (20 mL), KOAc (1.70 g, 17.58 mmol) was added at RT and the reaction mixture was purged with argon for 30 min. To the resulting reaction mixture, PdCl₂(dppf) (0.51 g, 0.70 mmol) was added and the reaction mixture was again purged with argon for 20 min. The reaction mixture was stirred in a sealed tube at 100° C. for 16 h. The progress of the reaction was monitored by TLC. After completion of reaction, the reaction mixture was filtered through Celite®. The filtrate was diluted with cold water and extracted with ethyl acetate. Combined organic layer was washed with water and brine, dried over anhydrous Na₂SO₄ and concentrated under reduced pressure. The crude compound was purified by silica gel column chromatography (40% EtOAc/hexane) to afford tert-butyl 4-[3-(4,4,5,5-tetramethyl-1,3,2-dioxaborolan-2-yl)phenyl]piperazine-1-carboxylate (1.5 g, 66%) as off white solid. LC-MS: m/z 389.2 [M+H]⁺.

### Step-3: tert-butyl 4-[3-(2,6-dibenzyloxy-3-pyridyl)phenyl]piperazine-1-carboxylate

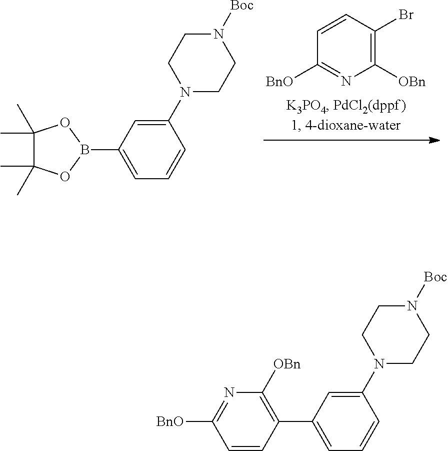 [SMILES: CC(C)(C)OC(=O)N1CCN(c2cccc(B3OC(C)(C)C(C)(C)O3)c2)CC1 ; Brc1ccc(OCc2ccccc2)nc1OCc1ccccc1 ; CC(C)(C)OC(=O)N1CCN(c2cccc(-c3ccc(OCc4ccccc4)nc3OCc3ccccc3)c2)CC1]

To a stirred solution of tert-butyl 4-[3-(4,4,5,5-tetramethyl-1,3,2-dioxaborolan-2-yl)phenyl]piperazine-1-carboxylate (1.30 g, 3.35 mmol, from step 2) and 2,6-dibenzyloxy-3-bromo-pyridine (1.48 g, 4.02 mmol) in 1,4-dioxane (13 mL), K₃PO₄ (2.11 g, 10.05 mmol) was added dissolved in H₂O (3 mL) at RT. The reaction mixture was purged with argon for 30 min. To the resulting reaction mixture, PdCl₂(dppf) (0.32 g, 0.40 mmol) was added and the reaction mixture was again purged with argon for 20 min. The reaction mixture was stirred in a sealed tube at 100° C. for 16 h. The progress of the reaction was monitored by TLC. After completion of reaction, the reaction mixture was filtered through Celite®. The filtrate was diluted with cold water and extracted with ethyl acetate. Combined organic layer was washed with water and brine, dried over anhydrous Na₂SO₄ and concentrated under reduced pressure. The crude compound was purified by silica gel column chromatography (50% EtOAc/hexane) to afford tert-butyl 4-[3-(2,6-dibenzyloxy-3-pyridyl)phenyl]piperazine-1-carboxylate (1.10 g, 59%) as an off white solid. ¹H NMR (400 MHz, DMSO-d₆) δ=7.76 (d, J=8.3 Hz, 1H), 7.48-7.35 (m, 10H), 7.23 (t, J=8.1 Hz, 1H), 7.12 (s, 1H), 6.97 (d, J=7.3 Hz, 1H), 6.86 (d, J=8.8 Hz, 1H), 6.55 (d, J=8.3 Hz, 1H), 5.39 (d, J=13.2 Hz, 4H), 3.40 (brs, 4H), 2.98 (brs, 4H), 1.43 (s, 9H). LC-MS: m/z 552.3 [M+H]⁺.

### Step-4: tert-butyl 4-[3-(2,6-dioxo-3-piperidyl)phenyl]piperazine-1-carboxylate

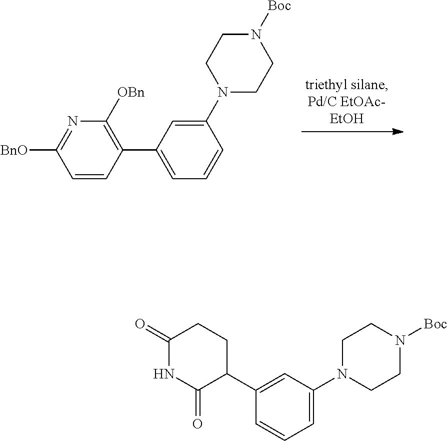 [SMILES: CC(C)(C)OC(=O)N1CCN(c2cccc(-c3ccc(OCc4ccccc4)nc3OCc3ccccc3)c2)CC1 ; CC(C)(C)OC(=O)N1CCN(c2cccc(C3CCC(=O)NC3=O)c2)CC1]

To a stirred solution of tert-butyl 4-[3-(2,6-dibenzyloxy-3-pyridyl)phenyl]piperazine-1-carboxylate (0.25 g, 0.45 mmol, step 3) in EtOAc (3 mL) and EtOH (3 mL), Pd/C (0.19 g) and triethyl silane (0.51 g, 4.5 mmol) were added at RT. The reaction mixture was stirred at RT for 72 h. The progress of the reaction was monitored by TLC. After completion of reaction, the reaction mixture was filtered through Celite®. The filtrate was concentrated under reduced pressure. The crude compound was purified by silica gel column chromatography (40% EtOAc/hexane) to afford tert-butyl 4-[3-(2,6-dioxo-3-piperidyl)phenyl]piperazine-1-carboxylate (0.10 g, 59%) as brown solid. LC-MS: m/z 374.45 [M+H]⁺.

### Step-5: 3-(3-piperazin-1-ylphenyl)piperidine-2,6-dione

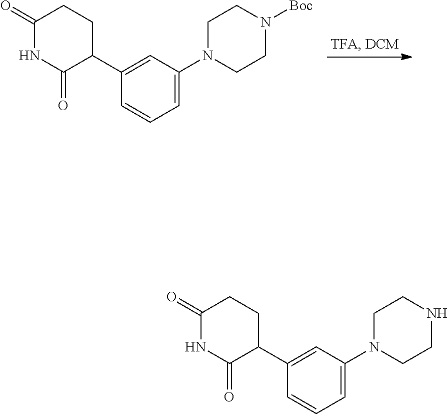 [SMILES: CC(C)(C)OC(=O)N1CCN(c2cccc(C3CCC(=O)NC3=O)c2)CC1 ; O=C1CCC(c2cccc(N3CCNCC3)c2)C(=O)N1]

To stirred solution of tert-butyl 4-[3-(2,6-dioxo-3-piperidyl)phenyl]piperazine-1-carboxylate (0.10 g, 0.26 mmol, from step 4) in DCM (2 mL) and TFA (0.92 g, 0.80 mmol) was added and the reaction mixture was stirred at 0° C. for 5 min followed by at RT for 2 h. The progress of the reaction was monitored by TLC. After completion of reaction, the reaction mixture was concentrated under reduced pressure to afford 3-(3-piperazin-1-ylphenyl)piperidine-2,6-dione (0.10 g, crude) as an off white solid which was used as such for the next reaction. ¹H NMR (400 MHz, DMSO-d₆) δ=10.81 (s, 1H), 7.23-7.19 (m, 1H), 6.91-6.84 (m, 2H), 6.72 (d, J=7.8 Hz, 1H), 3.78 (dd, J=4.9, 11.2 Hz, 1H), 3.32 (d, J=4.4 Hz, 4H), 3.23 (brs, 5H), 2.70-2.61 (m, 1H), 2.47-2.13 (m, 2H), 2.05-1.98 (m, 1H).

### Step-6: 3-[3-[4-[[4-[(3R,5R)-5-[(5-bromo-1-methyl-6-oxo-pyridazin-4-yl)amino]-1-methyl-3-piperidyl]phenyl]methyl]piperazin-1-yl]phenyl]piperidine-2,6-dione

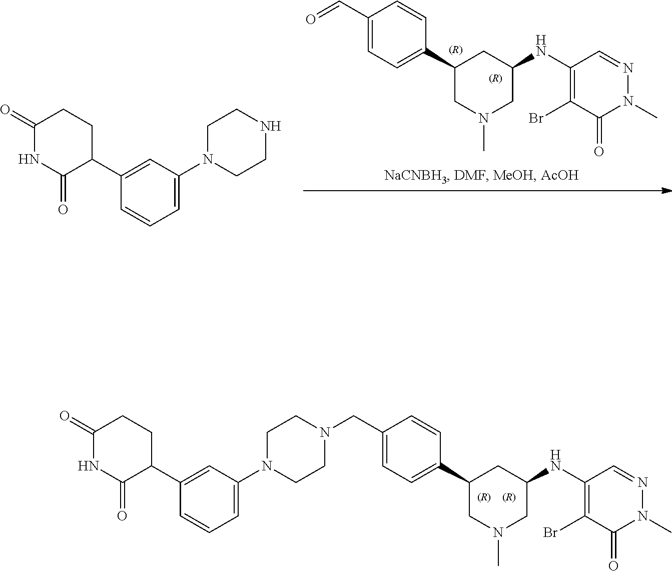

To a stirred solution of 3-(3-piperazin-1-ylphenyl)piperidine-2,6-dione (0.10 g, 0.36 mmol, from step 5) in DMF (3 mL) was added 4-[(3R,5R)-5-[(5-bromo-1-methyl-6-oxo-pyridazin-4-yl)amino]-1-methyl-3-piperidyl]benzaldehyde (0.14 g, 0.36 mmol, intermediate 4) in MeOH (5 mL) and DMSO (1 mL). To the resulting reaction mixture, AcOH (0.5 mL) and NaCNBH₃ (0.04 g, 0.72 mmol) were added and the reaction mixture was stirred at RT for 16 h. The progress of the reaction was monitored by TLC. After completion of reaction, all volatiles were evaporated. The reaction mixture was quenched with saturated NaHCO₃ solution and the aqueous layer was extracted with DCM. The crude compound was purified by prep HPLC (Method E) to afford 3-[3-[4-[[4-[(3R,5R)-5-[(5-bromo-1-methyl-6-oxo-pyridazin-4-yl)amino]-1-methyl-3-piperidyl]phenyl]methyl]piperazin-1-yl]phenyl]piperidine-2,6-dione (0.03 g, 11%) as an off white solid. ¹H NMR (400 MHz, DMSO-d₆) δ=10.79 (s, 1H), 7.86 (s, 1H), 7.34-7.23 (m, 4H), 7.15 (t, J=7.8 Hz, 1H), 6.83-6.77 (m, 2H), 6.62 (d, J=6.4 Hz, 1H), 6.07-5.88 (m, 1H), 4.06-3.99 (m, 1H), 3.75 (dd, J=5.0, 11.3 Hz, 1H), 3.61 (s, 3H), 3.53-3.47 (m, 2H), 3.24-2.98 (m, 8H), 2.61 (dd, J=5.3, 11.7 Hz, 2H), 2.54 (brs, 3H), 2.44 (brs, 3H), 2.24-2.12 (m, 2H), 2.09-1.98 (m, 3H), 1.89-1.76 (m, 1H); LC-MS: m/z 662.1 and 664.1 [M+H]⁺.

### Example 3: 3-[4-[2-[4-[(3R,5R)-5-[(5-bromo-1-methyl-6-oxo-pyridazin-4-yl)amino]-1-methyl-3-piperidyl]benzoyl]-2,7-diazaspiro[4.4]nonan-7-yl]phenyl]piperidine-2,6-dione (Compound 90)

### Step-1: tert-butyl 9-(4-bromophenyl)-3,9-diazaspiro[5.5]undecane-3-carboxylate

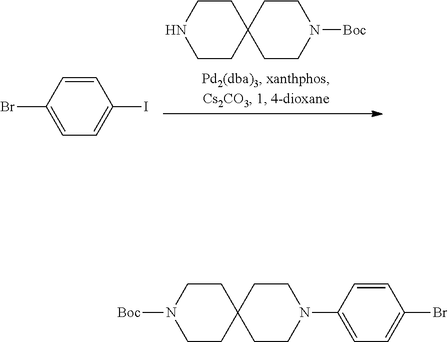 [SMILES: CC(C)(C)OC(=O)N1CCC2(CCNCC2)CC1 ; Brc1ccc(I)cc1 ; CC(C)(C)OC(=O)N1CCC2(CC1)CCN(c1ccc(Br)cc1)CC2]

To a stirred solution of 1-bromo-4-iodo-benzene (2.00 g, 7.09 mmol) and tert-butyl 3,9-diazaspiro[5.5]undecane-3-carboxylate (2.00 g, 8.00 mmol) in 1,4-dioxane (35 mL), Cs₂CO₃ (6.3 g, 19.38 mmol) was added at RT. The reaction mixture was purged with argon for 30 min. To the resulting reaction mixture, Pd₂(dba)₃ (0.64 g, 0.70 mmol) and xantphos-Pd (0.80 g, 1.40 mmol) were added and the reaction mixture was again purged with argon for 20 min. The reaction mixture was stirred in a sealed tube at 110° C. for 16 h. The progress of the reaction was monitored by TLC. After completion of reaction, the reaction mixture was filtered through Celite®. The filtrate was diluted with cold water and extracted with ethyl acetate. Combined organic layer was washed with water and brine, dried over anhydrous Na₂SO₄ and concentrated under reduced pressure. The crude compound was purified by silica gel column chromatography (7% EtOAc/hexane) to afford tert-butyl 9-(4-bromophenyl)-3,9-diazaspiro[5.5]undecane-3-carboxylate (0.90 g, 32%) as an off white solid. LC-MS: m/z 410.85 [M+H]⁺.

### Step-2: tert-butyl 9-[4-(2,6-dibenzyloxy-3-pyridyl)phenyl]-3,9-diazaspiro[5.5]undecane-3-carboxylate

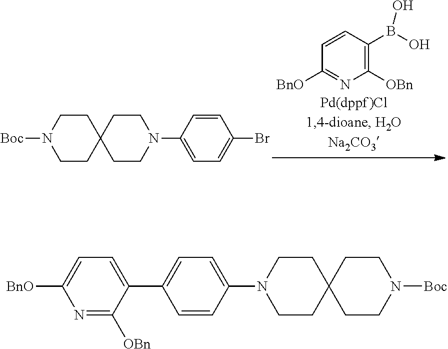 [SMILES: OB(O)c1ccc(OCc2ccccc2)nc1OCc1ccccc1 ; CC(C)(C)OC(=O)N1CCC2(CC1)CCN(c1ccc(Br)cc1)CC2 ; CC(C)(C)OC(=O)N1CCC2(CC1)CCN(c1ccc(-c3ccc(OCc4ccccc4)nc3OCc3ccccc3)cc1)CC2]

To a stirred solution of tert-butyl 9-(4-bromophenyl)-3,9-diazaspiro[5.5]undecane-3-carboxylate (1.0 g, 2.45 mmol, from step 1) and 2,6-dibenzyloxy-3-(4,4,5,5-tetramethyl-1,3,2-dioxaborolan-2-yl)pyridine (1.24 g, 3.71 mmol) in 1,4-dioxane (40 mL), Na₂CO₃ (0.52 g, 4.95 mmol) was added dissolved in H₂O (10 mL) at RT. The reaction mixture was purged with argon for 30 min. To the resulting reaction mixture, PdCl₂(dppf) (0.20 g, 0.02 mmol) was added and the reaction mixture was again purged with argon for 20 min. The reaction mixture was stirred in a sealed tube at 110° C. for 16 h. The progress of the reaction was monitored by TLC. After completion of reaction, the reaction mixture was filtered through Celite®. The filtrate was diluted with cold water and extracted with ethyl acetate. Combined organic layer was washed with water and brine, dried over anhydrous Na₂SO₄ and concentrated under reduced pressure. The crude compound was purified by silica gel column chromatography (10% EtOAc/hexane) to afford pure tert-butyl 9-[4-(2,6-dibenzyloxy-3-pyridyl)phenyl]-3,9-diazaspiro[5.5]undecane-3-carboxylate (0.6 g, 40%) as colorless liquid. LC-MS: m/z 620.79 [M+H]⁺.

### Step-3: tert-butyl 7~ [4-(2,6-dioxo-3-piperidyl)phenyl]-2,7-diazaspiro[4.4]nonane-2 carboxylate

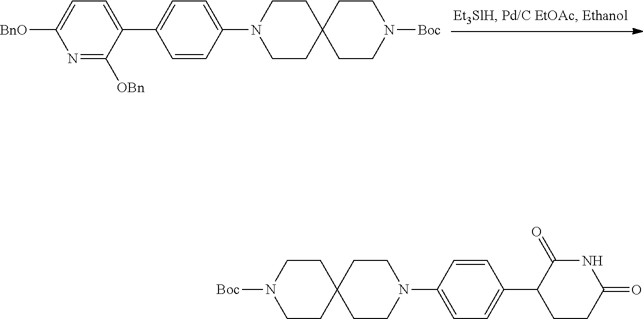 [SMILES: CC(C)(C)OC(=O)N1CCC2(CC1)CCN(c1ccc(-c3ccc(OCc4ccccc4)nc3OCc3ccccc3)cc1)CC2 ; CC(C)(C)OC(=O)N1CCC2(CC1)CCN(c1ccc(C3CCC(=O)NC3=O)cc1)CC2]

To stirred solution of tert-butyl 9-[4-(2,6-dibenzyloxy-3-pyridyl)phenyl]-3,9-diazaspiro[5.5]undecane-3-carboxylate (0.60 g, 0.96 mmol, from step 2) in EtOAc (6.6 mL) and EtOH (6.6 mL), Pd/C (0.49 g) and triethyl silane (1.20 g, 0.96 mmol) were added at RT. The reaction mixture was stirred at RT for 16 h. The progress of the reaction was monitored by TLC. After completion of reaction, the reaction mixture was filtered through Celite®. The filtrate was concentrated under reduced pressure. The crude compound was purified by silica gel column chromatography (2% MeOH/DCM) to afford tert-butyl 9-[4-(2,6-dioxo-3-piperidyl)phenyl]-3,9-diazaspiro[5.5]undecane-3-carboxylate (0.32 g, 73%) as an off white solid. LC-MS: m/z 442.08 [M+H]⁺.

### Step-4: 3-[4-(3,9-diazaspiro[5.5]undecan-3-yl)phenyl]piperidine-2,6-dione

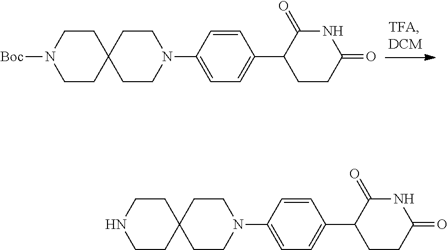 [SMILES: CC(C)(C)OC(=O)N1CCC2(CC1)CCN(c1ccc(C3CCC(=O)NC3=O)cc1)CC2 ; O=C1CCC(c2ccc(N3CCC4(CCNCC4)CC3)cc2)C(=O)N1]

A mixture of tert-butyl 9-[4-(2,6-dioxo-3-piperidyl)phenyl]-3,9-diazaspiro[5.5]undecane-3-carboxylate (0.40 g, 0.90 mmol, from step 3) in DCM (10 mL) and 30% TFA in DCM (0.6 mL) was stirred at 0° C. for 5 min followed by at RT for 2 h. The progress of the reaction was monitored by TLC. After completion of reaction, the reaction mixture was concentrated under reduced pressure to afford 3-[4-(3,9-diazaspiro[5.5]undecan-3-yl)phenyl]piperidine-2,6-dione (0.35 g, TFA salt) as white solid which was used as such for the next reaction. LC-MS: m/z 342.05 [M+H]⁺.

### Step-5: 3~ [4-[3-[4~ [(3R,5R)-5~ [(5-bromo-1-methyl-6-oxo-pyridazin-4-yl)amino]-1-methyl-3-piperidyl]benzoyl]-3,9-diazaspiro[5.5]undecan-9-yl]phenyl]piperidine-2,6-dione

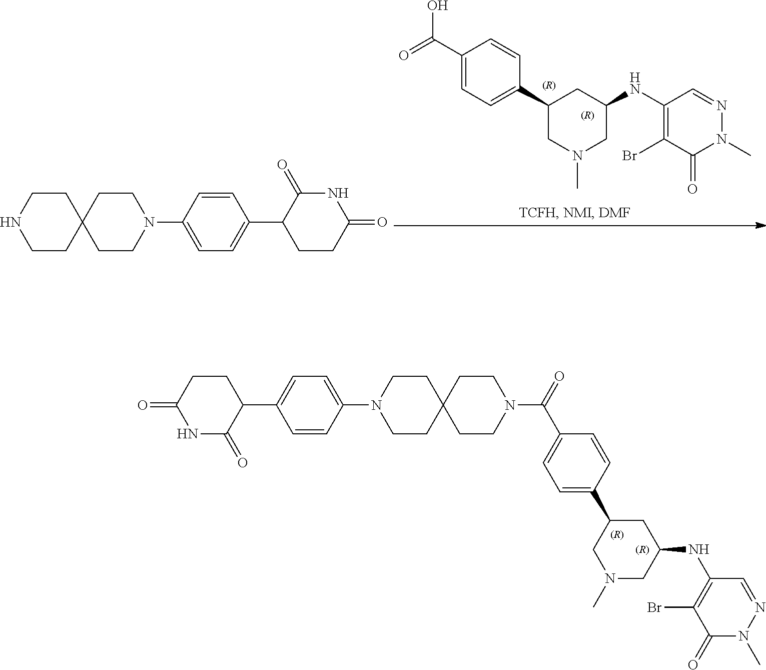

To a stirred solution of 3-[4-(3,9-diazaspiro[5.5]undecan-3-yl)phenyl]piperidine-2,6-dione (0.08 g, 0.23 mmol, from step 4) in DMF (1.5 mL), 4-[(3R,5R)-5-[(5-bromo-1-methyl-6-oxo-pyridazin-4-yl)amino]-1-methyl-3-piperidyl]benzoic acid (0.09 g, 0.23 mmol, intermediate 1) was added followed by N-methyl imidazole (0.09 g, 1.7 mmol) and the reaction mixture was stirred at RT for 10 min. To the resulting reaction mixture, N,N,N′,N′-tetramethylchloroformamidinium hexafluorophosphate (0.09 g, 0.35 mmol) was added and stirred at 85° C. for 16 h. The progress of the reaction was monitored by TLC. After completion of reaction, the reaction mixture was diluted with ice cold water. The precipitated solid was filtered to yield crude compound which was purified by prep HPLC (Method E) to afford 3-[4-[3-[4-[(3R,5R)-5-[(5-bromo-1-methyl-6-oxo-pyridazin-4-yl)amino]-1-methyl-3-piperidyl]benzoyl]-3,9-diazaspiro[5.5]undecan-9-yl]phenyl]piperidine-2,6-dione (0.06 g, 35%) as an off white solid. ¹H NMR (400 MHz, DMSO-d₆) δ=10.76 (s, 1H), 7.87 (s, 1H), 7.33 (s, 4H), 7.03 (d, J=8.8 Hz, 2H), 6.89 (d, J=8.8 Hz, 2H), 5.81 (d, J=9.0 Hz, 1H), 4.00-3.91 (m, 1H), 3.71 (dd, J=5.0, 10.9 Hz, 1H), 3.59 (s, 5H), 3.13 (brs, 4H), 3.03-2.87 (m, 4H), 2.64-2.59 (m, 2H), 2.43 (d, J=4.6 Hz, 1H), 2.25 (s, 3H), 2.15-1.98 (m, 5H), 1.72 (d, J=11.6 Hz, 1H), 1.60 (brs, 4H), 1.54-1.44 (m, 4H); LC-MS: m/z 744 and 746 [M+H]⁺.

### Example 4 & 5; one of which is (R)-3-(4-(9-(4-((3R,5R)-5-((5-bromo-1-methyl-6-oxo-1,6-dihydropyridazin-4-yl)amino)-1-methylpiperidin-3-yl)benzoyl)-3,9-diazaspiro[5.5]undecan-3-yl)phenyl)piperidine-2,6-dione and the other is(S)-3-(4-(9-(4-((3R,5R)-5-((5-bromo-1-methyl-6-oxo-1,6-dihydropyridazin-4-yl)amino)-1-methylpiperidin-3-yl)benzoyl)-3,9-diazaspiro[5.5]undecan-3-yl)phenyl)piperidine-2,6-dione (Compounds 239 & 240)

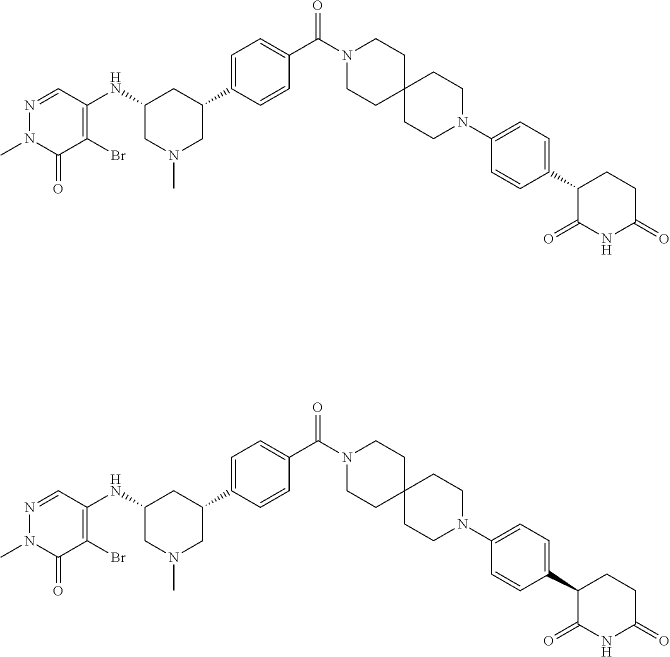 [SMILES: CN1C[C@H](Nc2cnn(C)c(=O)c2Br)C[C@H](c2ccc(C(=O)N3CCC4(CC3)CCN(c3ccc([C@@H]5CCC(=O)NC5=O)cc3)CC4)cc2)C1 ; CN1C[C@H](Nc2cnn(C)c(=O)c2Br)C[C@H](c2ccc(C(=O)N3CCC4(CC3)CCN(c3ccc([C@H]5CCC(=O)NC5=O)cc3)CC4)cc2)C1]

The stereoisomers of 3-(4-(9-(4-((3R,5R)-5-((5-bromo-1-methyl-6-oxo-1,6-dihydropyridazin-4-yl)amino)-1-methylpiperidin-3-yl)benzoyl)-3,9-diazaspiro[5.5]undecan-3-yl)phenyl)piperidine-2,6-dione (0.40 g, example 3) were separated by chiral prep-SFC using CHIRAL PAKIG (30 mm×250 mm×5 μm) column and DCM:MeOH (1:1) as mobile phase to afford (R)-3-(4-(9-(4-((3R,5R)-5-((5-bromo-1-methyl-6-oxo-1,6-dihydropyridazin-4-yl)amino)-1-methylpiperidin-3-yl)benzoyl)-3,9-diazaspiro[5.5]undecan-3-yl)phenyl)piperidine-2,6-dione (0.01 g) and(S)-3-(4-(9-(4-((3R,5R)-5-((5-bromo-1-methyl-6-oxo-1,6-dihydropyridazin-4-yl)amino)-1-methylpiperidin-3-yl)benzoyl)-3,9-diazaspiro[5.5]undecan-3-yl)phenyl)piperidine-2,6-dione (0.01 g) both as off-white solid.

Example 4 (first eluting isomer): 1H NMR (400 MHz, DMSO-d₆) δ=10.75 (s, 1H), 7.87 (s, 1H), 7.36-7.30 (m, 4H), 7.04-7.00 (m, 2H), 6.91-6.86 (m, 2H), 5.80 (d, J=9.0 Hz, 1H), 4.02-3.88 (m, 1H), 3.71 (dd, J=4.9, 10.8 Hz, 1H), 3.64-3.56 (m, 5H), 3.20-3.08 (m, 4H), 3.04-2.91 (m, 3H), 2.88-2.82 (m, 1H), 2.70-2.57 (m, 2H), 2.44-2.41 (m, 1H), 2.25 (s, 3H), 2.13-1.91 (m, 5H), 1.72 (q, J=12.2 Hz, 1H), 1.63-1.42 (m, 8H). LC-MS: m/z 744.83 and 746.75 [M+H]⁺.

Example 5 (second eluting isomer): 1H NMR (400 MHz, DMSO-d₆) δ=10.76 (s, 1H), 7.88 (s, 1H), 7.35-7.32 (m, 4H), 7.05-7.00 (m, 2H), 6.92-6.87 (m, 2H), 5.84-5.77 (m, 1H), 4.00-3.92 (m, 1H), 3.71 (dd, J=5.1, 11.0 Hz, 1H), 3.64-3.57 (m, 5H), 3.19-3.09 (m, 4H), 3.04-2.91 (m, 3H), 2.89-2.82 (m, 1H), 2.63-2.57 (m, 1H), 2.42-2.40 (m, 1H), 2.25 (s, 3H), 2.13-1.91 (m, 6H), 1.76-1.68 (m, 1H), 1.64-1.43 (m, 8H). LCMS: m/z 744.66 and 746.63 [M+H]⁺.

### Example 6: 3-[4-[3-[4-[(3R,5R)-5~ [(5-chloro-1-methyl-6-oxo-pyridazin-4-yl)amino]-1-methyl-3-piperidyl]benzoyl]-3,9-diazaspiro[5.5]undecan-9-yl]phenyl]piperidine-2,6-dione (Compound 145)

### Step-1: tert-butyl 9-(4-bromophenyl)-3,9-diazaspiro[5.5]undecane-3-carboxylate

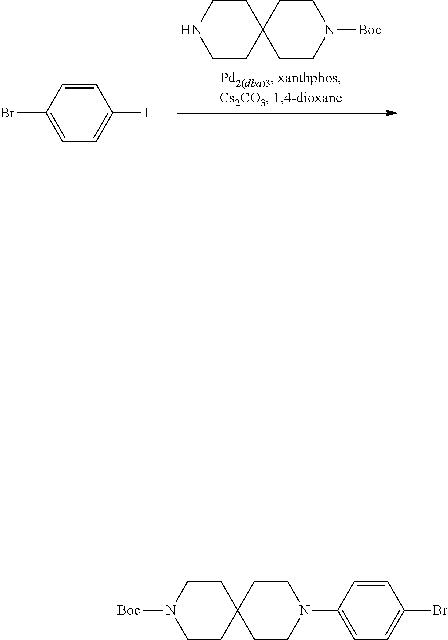 [SMILES: CC(C)(C)OC(=O)N1CCC2(CCNCC2)CC1 ; Brc1ccc(I)cc1 ; CC(C)(C)OC(=O)N1CCC2(CC1)CCN(c1ccc(Br)cc1)CC2]

To a stirred solution of 1-bromo-4-iodo-benzene (1.00 g, 3.54 mmol) in 1,4-dioxane (35 mL) were added tert-butyl 3,9-diazaspiro[5.5]undecane-3-carboxylate (1.00 g, 4.00 mmol) and Cs₂CO₃ (3.20 g, 9.84 mmol) at RT. The reaction mixture was purged with argon for 30 min. To the resulting reaction mixture, Pd₂(dba)₃ (0.32 g, 0.35 mmol) and xantphos-Pd (0.40 g, 0.70 mmol) were added at RT and the reaction mixture was again purged with argon for 20 min. The reaction mixture was stirred in a sealed tube at 110° C. for 16 h. The progress of the reaction was monitored by TLC. After completion of reaction, the reaction mixture was filtered through Celite®. The filtrate was diluted with cold water and extracted with ethyl acetate. Combined organic layer was washed with water and brine, dried over anhydrous Na₂SO₄ and concentrated under reduced pressure to afford tert-butyl 9-(4-bromophenyl)-3,9-diazaspiro[5.5]undecane-3-carboxylate (0.80 g, crude) as an off white solid; which was used as such for the next reaction. LCMS: m/z 409.30 and 411.20 [M+H]⁺. Step-2: tert-butyl 9-[4-(2,6-dibenzyloxy-3-pyridyl)phenyl]-3,9-diazaspiro[5.5]undecane-3-carboxylate

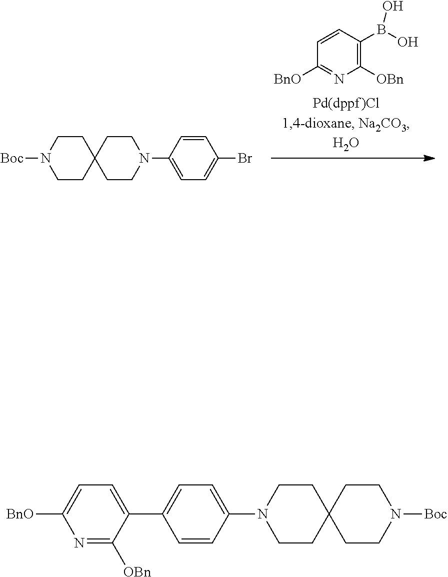 [SMILES: OB(O)c1ccc(OCc2ccccc2)nc1OCc1ccccc1 ; CC(C)(C)OC(=O)N1CCC2(CC1)CCN(c1ccc(Br)cc1)CC2 ; CC(C)(C)OC(=O)N1CCC2(CC1)CCN(c1ccc(-c3ccc(OCc4ccccc4)nc3OCc3ccccc3)cc1)CC2]

To a stirred solution of tert-butyl 9-(4-bromophenyl)-3,9-diazaspiro[5.5]undecane-3-carboxylate (0.60 g, 1.47 mmol, from stop 1) in 1,4-dioxane (20 mL) was added (2,6-dibenzyloxy-3-pyridyl) boronic acid (0.90 g, 2.17 mmol) followed by Na₂CO₃ (0.30 g, 2.94 mmol) at RT. The reaction mixture was purged with argon for 30 min. To the resulting reaction mixture, PdCl₂(dppf) (0.11 g, 0.14 mmol) was added at RT and the reaction mixture was again purged with argon for 20 min. The reaction mixture was stirred in sealed tube at 100° C. for 16 h. The progress of the reaction was monitored by TLC. After completion of reaction, the reaction mixture was filtered through Celite®. The filtrate was diluted with cold water and extracted with ethyl acetate. Combined organic layer was washed with water and brine, dried over anhydrous Na₂SO₄ and concentrated under reduced pressure to afford tert-butyl 9-[4-(2,6-dibenzyloxy-3-pyridyl)phenyl]-3,9-diazaspiro[5.5]undecane-3-carboxylate (0.60 g. crude) as a red liquid, which was used as such for the next reaction. LCMS: m/z 620.20 [M+H]⁺.

### Step-3: tert-butyl 9-[4-(2,6-dioxo-3-piperidyl)phenyl]-3,9-diazaspiro[5.5]undecane-3-carboxylate

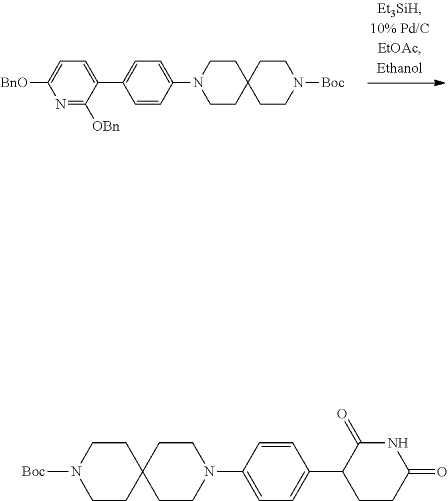 [SMILES: CC(C)(C)OC(=O)N1CCC2(CC1)CCN(c1ccc(-c3ccc(OCc4ccccc4)nc3OCc3ccccc3)cc1)CC2 ; CC(C)(C)OC(=O)N1CCC2(CC1)CCN(c1ccc(C3CCC(=O)NC3=O)cc1)CC2]

To a stirred solution of tert-butyl 9-[4-(2,6-dibenzyloxy-3-pyridyl)phenyl]-3,9-diazaspiro[5.5]undecane-3-carboxylate (0.60 g, 0.96 mmol, from step 2) in EtOAc (1 mL) and EtOH (1 mL) were added triethyl silane (1.60 g, 14.40 mmol) and 10% Pd/C (0.39 g) at RT. The reaction mixture was stirred in a sealed tube at RT for 24 h. The progress of the reaction was monitored by TLC and LCMS. After completion of reaction, the reaction mixture was filtered through Celite®, The filtrate was concentrated under reduced pressure. The crude compound was purified by silica gel column chromatography (5% MeOH in DCM) to afford tert-butyl 9-[4-(2,6-dioxo-3-piperidyl)phenyl]-3,9-diazaspiro[5.5]undecane-3-carboxylate (0.25 g, 62%) as a brown solid. LCMS: m/z 442.50 [M+H]⁺.

### Step-4: 3-[4-(3,9-diazaspiro[5.5]undecan-3-yl)phenyl]piperidine-2,6-dione

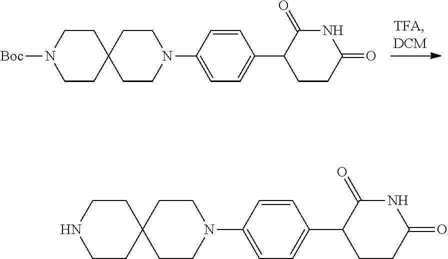 [SMILES: CC(C)(C)OC(=O)N1CCC2(CC1)CCN(c1ccc(C3CCC(=O)NC3=O)cc1)CC2 ; O=C1CCC(c2ccc(N3CCC4(CCNCC4)CC3)cc2)C(=O)N1]

A mixture of tert-butyl 9-[4-(2,6-dioxo-3-piperidyl)phenyl]-3,9-diazaspiro[5.5]undecane-3-carboxylate (0.63 g, 1.42 mmol, from step 3) and TFA (0.5 mL) in DCM (0.5 mL) was stirred at 0° C. for 5 min followed by at RT for 1 h. The progress of the reaction was monitored by TLC. After completion of reaction, the reaction mixture was concentrated under reduced pressure to afford 3-[4-(3,9-diazaspiro[5.5]undecan-3-yl)phenyl]piperidine-2,6-dione (TFA salt, 0.40 g) as brown solid; which was used as such for the next reaction.

LCMS: m/z 342.60 [M+H]⁺

### Step-5: 3-[4-[3-[4-[(3R,5R)-5-[(5-chloro-1-methyl-6-oxo-pyridazin-4-yl)amino]-1-methyl-3-piperidyl]benzoyl]-3,9-diazaspiro[5.5]undecan-9-yl]phenyl]piperidine-2,6-dione

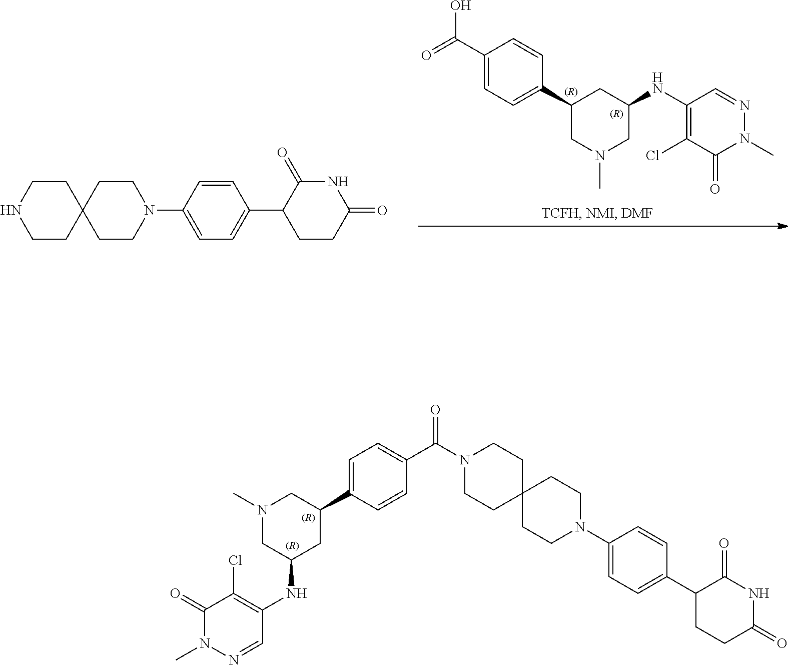

To a stirred solution of 4-[(3R,5R)-5-[(5-chloro-1-methyl-6-oxo-pyridazin-4-yl)amino]-1-methyl-3-piperidyl]benzoic acid (0.11 g, 0.29 mmol, intermediate 8) in DMF (2 mL), N-methyl imidazole (0.12 g, 0.29 mmol) was added followed by N,N,N′,N′-tetramethylchloroformamidinium hexafluorophosphate (0.12 g, 0.43 mmol) at RT. The reaction mixture was stirred at RT for 10 min. To this was added 3-[4-(3,9-diazaspiro[5.5]undecan-3-yl) phenyl]piperidine-2,6-dione (0.10 g, 0.29 mmol, from step 4) and the reaction mixture was stirred at 80° C. for 8 h. The progress of the reaction was monitored by TLC. After completion of reaction, the reaction mixture was diluted with ice-cold water and extracted with ethyl acetate. The combined organic layer was dried over anhydrous Na₂SO₄ and concentrated under reduced pressure, the crude compound was purified by prep HPLC (Method E) to afford 3-[4-[3-[4-[(3R,5R)-5-[(5-chloro-1-methyl-6-oxo-pyridazin-4-yl)amino]-1-methyl-3-piperidyl]benzoyl]-3,9-diazaspiro[5.5]undecan-9-yl]phenyl]piperidine-2,6-dione (0.02 g) as an off-white solid. 1H NMR (400 MHz, DMSO-d₆) δ=10.75 (s, 1H), 7.96 (s, 1H), 7.33 (d, J=1.8 Hz, 4H), 7.03 (d, J=8.8 Hz, 2H), 6.89 (d, J=8.8 Hz, 2H), 6.11 (d, J=8.9 Hz, 1H), 3.97-3.92 (m, 1H), 3.71 (dd, J=4.9, 10.8 Hz, 1H), 3.65-3.61 (m, 1H), 3.58 (s, 4H), 3.38-3.33 (m, 1H), 3.28-3.22 (m, 1H), 3.13 (brs, 4H), 3.04-2.92 (m, 2H), 2.86 (d, J=10.3 Hz, 1H), 2.63-2.57 (m, 1H), 2.47-2.34 (m, 2H), 2.25 (s, 3H), 2.05-1.87 (m, 4H), 1.75-1.66 (m, 1H), 1.60 (brs, 4H), 1.52-1.39 (m, 4H); LCMS: m/z 700.20 [M+H]⁺.

### Example 7: 3-[4-[3-[4-[(3R,5R)-5-[(5-bromo-1-methyl-6-oxo-pyridazin-4-yl)amino]-1-methyl-3-piperidyl]benzoyl]-3,9-diazaspiro[5.5]undecan-9-yl]-2-fluoro-phenyl]piperidine-2,6-dione (Compound 157)

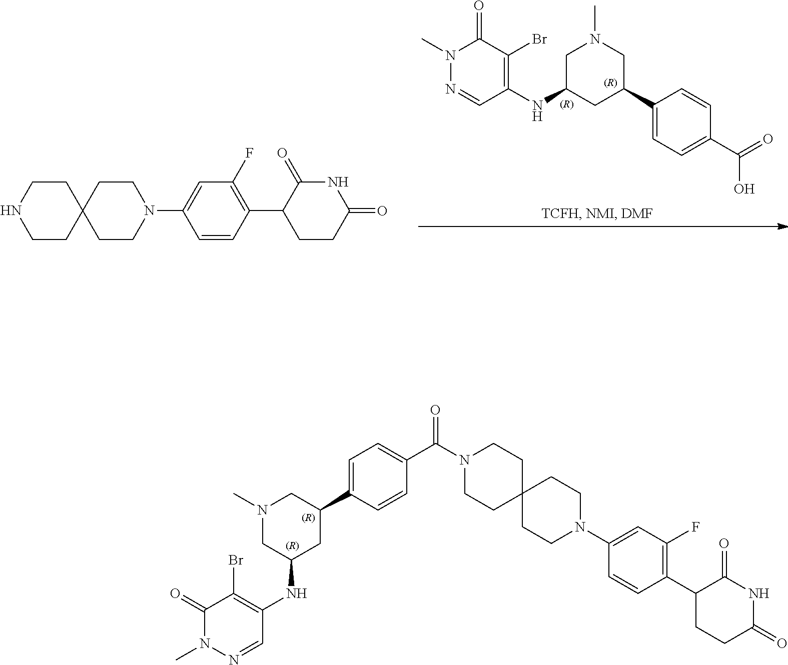

To a stirred solution of 4-[(3R,5R)-5-[(5-bromo-1-methyl-6-oxo-pyridazin-4-yl)amino]-1-methyl-3-piperidyl]benzoic acid (0.10 g, 0.24 mmol, intermediate 1) in DMF (3 mL), N-methyl imidazole (0.09 g, 1.11 mmol) was added followed by N, N,N′,N′-tetramethylchloroformamidinium hexafluorophosphate (0.09 g, 0.33 mmol) at RT. The reaction mixture was stirred at RT for 10 min. To this was added 3-[4-(3,9-diazaspiro[5.5]undecan-3-yl)-2-fluoro-phenyl]piperidine-2,6-dione (0.08 g, 0.22 mmol) at RT. The reaction mixture was stirred at 80° C. for 16 h. The progress of the reaction was monitored by TLC. After completion of reaction, the reaction mixture was diluted with ice-cold water and extracted with ethyl acetate. The combined organic layer was dried over anhydrous Na₂SO₄ and concentrated under reduced pressure, the crude compound was purified by prep HPLC (Method E) to afford 4-((3R,5R)-5-((5-bromo-1-methyl-6-oxo-1,6-dihydropyridazin-4-yl)amino)-1-methylpiperidin-3-yl)-N-(4-((2-(2,6-dioxopiperidin-3-yl)-1,3-dioxoisoindolin-4-yl)oxy)butyl)benzamide (0.008 g) as an off white solid. ¹H NMR (400 MHz, DMSO-d₆) δ=7.88 (s, 1H), 7.33 (s, 4H), 7.06 (t, J=8.7 Hz, 1H), 6.71 (d, J=12.0 Hz, 2H), 5.81 (d, J=9.0 Hz, 1H), 4.00-3.92 (m, 1H), 3.87 (dd, J=4.9, 12.3 Hz, 1H), 3.59 (s, 4H), 3.31-3.25 (m, 1H), 3.17 (d, J=5.1 Hz, 4H), 3.04-2.91 (m, 2H), 2.85 (dd, J=2.9, 10.8 Hz, 1H), 2.76-2.65 (m, 1H), 2.25 (s, 3H), 2.13 (dd, J=3.9, 12.7 Hz, 1H), 2.03-1.89 (m, 4H), 1.72 (q, J=12.2 Hz, 1H), 1.64-1.40 (m, 8H), 4H merged in solvent peak; LCMS: m/z 762.59 and 764.48 [M+H]⁺.

### Example 8: 3-[6-[3-[4-[(3R,5R)-5-[(5-chloro-1-methyl-6-oxo-pyridazin-4-yl)amino]-1-methyl-3-piperidyl]benzoyl]-3,9-diazaspiro[5.5]undecan-9-yl]-1-methyl-indazol-3-yl]piperidine-2,6-dione (Compound 208)

### Step-1:6-bromo-3-iodo-1H-indazole

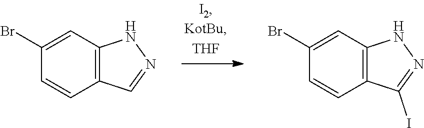 [SMILES: Brc1ccc2cn[nH]c2c1 ; Brc1ccc2c(I)n[nH]c2c1]

To a stirred solution of 6-bromo-1H-indazole (5.00 g, 25.37 mmol) in THF (75 mL) at 0° C. was added potassium tert-butoxide (5.87 g, 50.70 mmol) followed by iodine (19.40 g, 76.30 mmol) dropwise and reaction stirred at RT for 1 h. The progress of the reaction was monitored by TLC. After completion of reaction, reaction mixture was diluted with sodium thiosulfate solution and extracted with ethyl acetate. Combined organic layer was washed with water and brine, dried over anhydrous Na₂SO₄ and concentrated under reduced pressure to afford 6-bromo-3-iodo-1H-indazole (6.50 g, crude) as an off white solid, which was used as such for the next reaction. LC-MS: m/z 322.82 and 324.82 [M+H]⁺.

### Step-2: 6-bromo-3-iodo-1-methyl-indazole

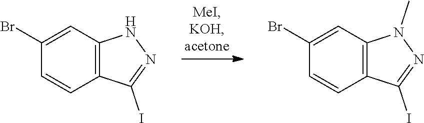

To a stirred solution of 6-bromo-3-iodo-1H-indazole (6.50 g, 20.00 mmol, from step 1) in acetone (100 mL) at 0° C. was added KOH (2.30 g, 41.00 mmol) and reaction mixture was stirred at RT for 5 min. To this was added iodomethane (7.20 g, 50.00 mmol) and reaction mixture was stirred at RT for 16 h. The progress of the reaction was monitored by TLC. After completion of reaction, the reaction mixture was diluted with cold water and extracted with DCM. Combined organic layer was washed with water and brine, dried over anhydrous Na₂SO₄ and concentrated under reduced pressure. Crude compound was purified by CombiFlash® column using (4% EtOAc/heptane) to afford 6-bromo-3-iodo-1-methyl-indazole (6.00 g, 88%) as an off white solid. LC-MS: m/z 336.87 and 338.87 [M+H]⁺.

### Step-3: 6-bromo-3-(2,6-dibenzyloxy-3-pyridyl)-1-methyl-indazole

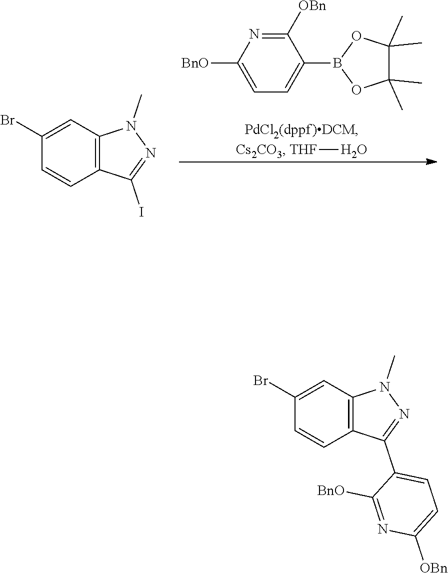

To a stirred solution of 6-bromo-3-iodo-1-methyl-indazole (6.00 g, 17.80 mmol, from step 2) in THF (70 mL) and water (60 mL) was added 2,6-dibenzyloxy-3-(4,4,5,5-tetramethyl-1,3,2-dioxaborolan-2-yl)pyridine (11.15 g, 26.72 mmol) followed by cesium carbonate (17.40 g, 53.40 mmol) and the reaction mixture was purged under nitrogen for 10 min. To this resulting mixture, 1,1′-bis(diphenylphosphino)ferrocene]dichloropalladium(II), dichloromethane complex (1.63 g, 1.78 mmol) was added at RT. The reaction mixture was stirred at 100° C. for 16 h. The progress of the reaction was monitored by TLC. After completion of reaction, the reaction mixture was cooled to room temperature, diluted with water and extracted with EtOAc. Combined organic layer was washed with water and brine, dried over anhydrous Na₂SO₄ and concentrated under reduced pressure. The crude compound was purified by CombiFlash® column chromatography (15% EtOAc/heptane) to afford 6-bromo-3-(2,6-dibenzyloxy-3-pyridyl)-1-methyl-indazole (3.80 g, 43%) as pale yellow solid. LC-MS: m/z 500.0 and 502.0 [M+H]⁺.

### Step-4: tert-butyl 9-[3-(2,6-dibenzyloxy-3-pyridyl)-1-methyl-indazol-6-yl]-3,9-diazaspiro[5.5]undecane-3-carboxylate

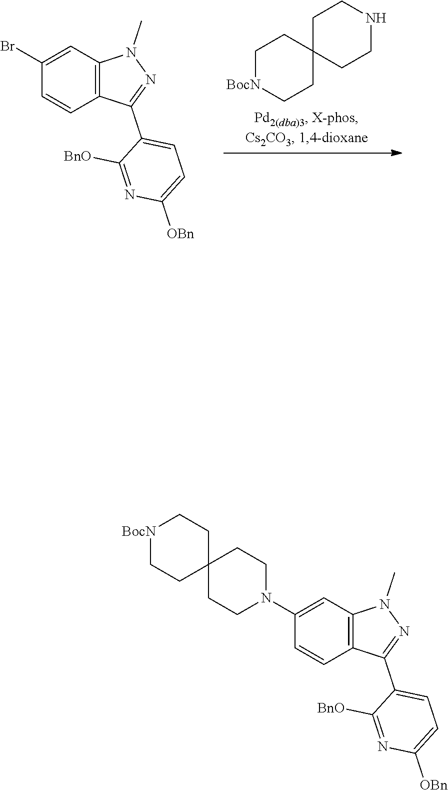 [SMILES: Cn1nc(-c2ccc(OCc3ccccc3)nc2OCc2ccccc2)c2ccc(Br)cc21 ; CC(C)(C)OC(=O)N1CCC2(CCNCC2)CC1 ; Cn1nc(-c2ccc(OCc3ccccc3)nc2OCc2ccccc2)c2ccc(N3CCC4(CCN(C(=O)OC(C)(C)C)CC4)CC3)cc21]

To a stirred solution of 6-bromo-3-(2,6-dibenzyloxy-3-pyridyl)-1-methyl-indazole (2.00 g, 3.99 mmol, from step 3) in 1,4-dioxane (40 mL) was added tert-butyl 3,9-diazaspiro[5.5]undecane-3-carboxylate (1.11 g, 4.39 mmol) followed by cesium carbonate (2.65 g, 8.13 mmol) at RT. The reaction mixture was purged with argon for 10 min. To the resulting reaction mixture, tris(dibenzylideneacetone) dipalladium (0) (0.36 g, 0.40 mmol) and 2-dicyclohexylphospbino-2′,4′,6′-triisopropylbiphenyl (0.39 g, 0.80 mmol) were added and the reaction mixture was again purged with argon for 2 min. The reaction mixture was stirred in a sealed tube at 100° C. for 16 h. The progress of the reaction was monitored by TLC. After completion of reaction, the reaction mixture was filtered through Celite®. The filtrate was diluted with cold water and extracted with ethyl acetate. Combined organic layer was washed with water and brine, dried over anhydrous Na₂SO₄ and concentrated under reduced pressure to afford crude compound which was purified by column chromatography using (40% EtOAc/heptane) to afford tert-butyl 9-[3-(2,6-dibenzyloxy-3-pyridyl)-1-methyl-indazol-6-yl]-3,9-diazaspiro[5.5]undecane-3-carboxylate (1.65 g, 61%) as an off white solid. LC-MS: m/z 674.40 [M+H]⁺.

### Step-5: tert-butyl 9-[3-(2,6-dioxo-3-piperidyl)-1-methyl-indazol-6-yl]-3,9-diazaspiro[5.5]undecane-3-carboxylate

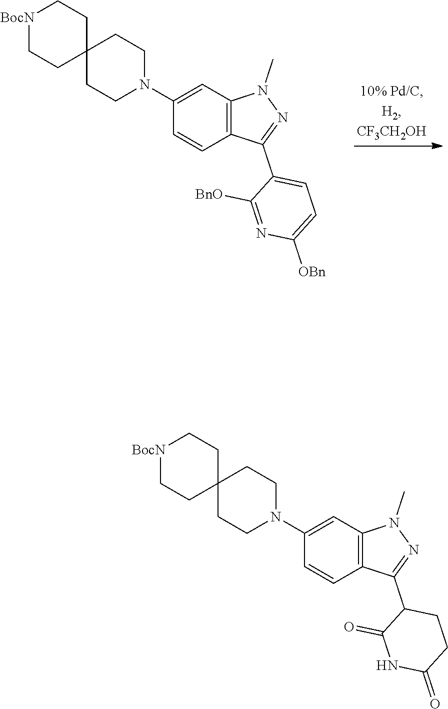 [SMILES: Cn1nc(-c2ccc(OCc3ccccc3)nc2OCc2ccccc2)c2ccc(N3CCC4(CCN(C(=O)OC(C)(C)C)CC4)CC3)cc21 ; Cn1nc(C2CCC(=O)NC2=O)c2ccc(N3CCC4(CCN(C(=O)OC(C)(C)C)CC4)CC3)cc21]

To a mixture of tert-butyl 9-[3-(2,6-dibenzyloxy-3-pyridyl)-1-methyl-indazol-6-yl]-3,9-diazaspiro[5.5]undecane-3-carboxylate (1.00 g, 1.48 mmol, from step 4) in 2,2,2-trifluoroethanol (15 mL) was added 10% Pd/C (1.00 g) and reaction mixture was stirred at RT under 100 psi hydrogen gas pressure in autoclave for 8 h. The progress of the reaction was monitored by TLC. After completion of reaction, the reaction mixture was filtered through Celite®. The filtrate was diluted with cold water and extracted with ethyl acetate. Combined organic layer was washed with water and brine, dried over anhydrous Na₂SO₄ and concentrated under reduced pressure to afford tert-butyl 9-[3-(2,6-dioxo-3-piperidyl)-1-methyl-indazol-6-yl]-3,9-diazaspiro[5.5]undecane-3-carboxylate (0.52 g, crude) as an off white solid, which was used such as for the next reaction. LC-MS: m/z 496.40 [M+H]⁺.

### Step-6: 3-[6-(3,9-diazaspiro[5.5]undecan-3-yl)-1-methyl-indazol-3-yl]piperidine-2,6-dione

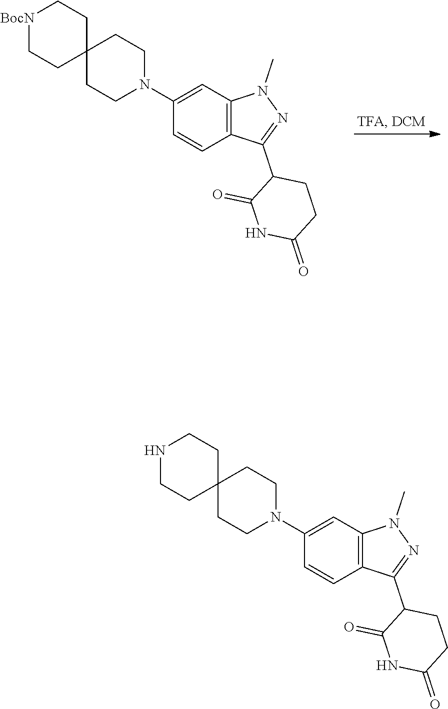 [SMILES: Cn1nc(C2CCC(=O)NC2=O)c2ccc(N3CCC4(CCN(C(=O)OC(C)(C)C)CC4)CC3)cc21 ; Cn1nc(C2CCC(=O)NC2=O)c2ccc(N3CCC4(CCNCC4)CC3)cc21]

To a stirred solution of tert-butyl 9-[3-(2,6-dioxo-3-piperidyl)-1-methyl-indazol-6-yl]-3,9-diazaspiro[5.5]undecane-3-carboxylate (0.50 g, 1.00 mmol, from step 5) in DCM (10 mL) was added TFA (1.16 g, 10.09 mmol) at 0° C. The reaction mixture was stirred at RT for 2 h. The progress of the reaction was monitored by TLC. After completion of reaction, the reaction mixture was concentrated under reduced pressure to afford 3-[6-(3,9-diazaspiro[5.5]undecan-3-yl)-1-methyl-indazol-3-yl]piperidine-2,6-dione (0.35 g, TFA salt) as an off white solid, which was used as such for the next reaction. LC-MS: m/z 396.40 [M+H]⁺.

### Step-7: 3-[6-[3-[4-[(3R,5R)-5-[(5-chloro-1-methyl-6-oxo-pyridazin-4-yl)amino]-1-methyl-3-piperidyl]benzoyl]-3,9-diazaspiro[5.5]undecan-9-yl]-1-methyl-indazol-3-yl]piperidine-2,6-dione

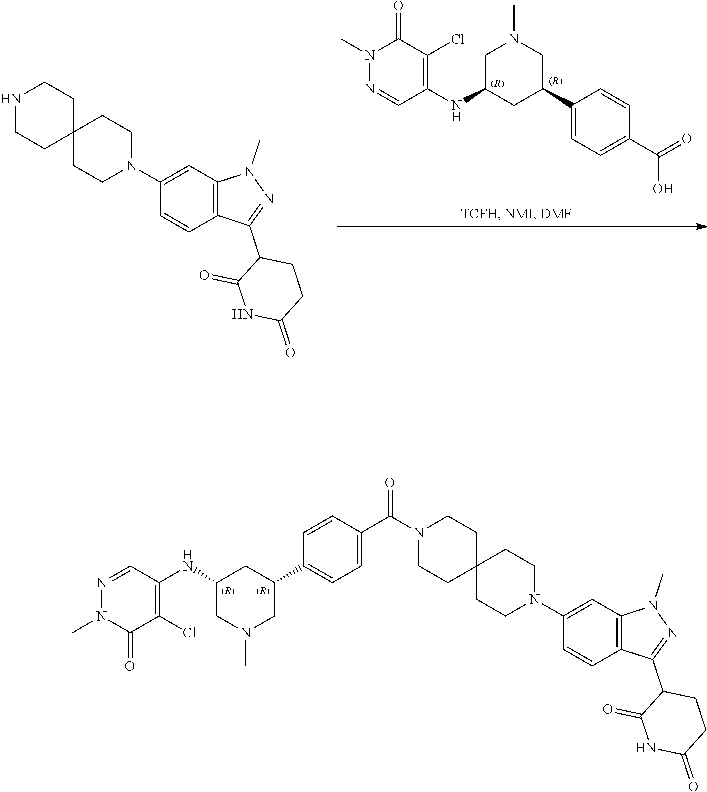

To a stirred solution of 4-[(3R,5R)-5-[(5-chloro-1-methyl-6-oxo-pyridazin-4-yl)amino]-1-methyl-3-piperidyl]benzoic acid (0.07 g, 0.18 mmol, intermediate 8) in DMF (2 mL) was added 1-methylimidazole (0.07 g, 0.92 mmol) followed by N,N,N′,N′-tetramethylchloroformamidinium hexafluorophosphate (0.08 g, 0.27 mmol) and the reaction mixture was stirred at RT for 10 min. To this was added 3-[6-(3,9-diazaspiro[5.5]undecan-3-yl)-1-methyl-indazol-3-yl]piperidine-2,6-dione (0.11 g, 0.27 mmol, from step 6) and the reaction mixture was stirred at 80° C. for 16 h. The progress of the reaction was monitored by TLC. After completion of reaction, the reaction mixture was concentrated under reduced pressure, the crude compound was purified by prep HPLC (Method E) to afford 3-[6-[3-[4-[(3~{R},5~{R})-5-[(5-chloro-1-methyl-6-oxo-pyridazin-4-yl)amino]-1-methyl-3-piperidyl]benzoyl]-3,9-diazaspiro[5.5]undecan-9-yl]-1-methyl-indazol-3-yl]piperidine-2,6-dione (0.03 g, 22%) as an off white solid. ¹H NMR (400 MHz, DMSO-d₆) δ=10.83 (s, 1H), 7.96 (s, 1H), 7.47 (d, J=9.0 Hz, 1H), 7.37-7.27 (m, 4H), 6.91 (d, J=9.0 Hz, 1H), 6.84 (s, 1H), 6.10 (d, J=8.9 Hz, 1H), 4.24 (dd, J=5.1, 9.0 Hz, 1H), 3.94 (dd, J=4.6, 9.0 Hz, 1H), 3.88 (s, 3H), 3.72-3.53 (m, 5H), 3.24 (brs, 4H), 3.04-2.82 (m, 3H), 2.66-2.56 (m, 2H), 2.35-2.21 (m, 4H), 2.19-2.11 (m, 1H), 2.07-1.87 (m, 3H), 1.76-1.69 (m, 1H), 1.65 (brs, 4H), 1.50 (dd, J=8.0, 13.9 Hz, 4H) [2H merged in solvent]. LC-MS: m/z 754.49 [M+H]⁺.

### Example 9: 3-[4-[3-[4-[(3R,5R)-5-[(5-chloro-1-methyl-6-oxo-pyridazin-4-yl)amino]-1-methyl-3-piperidyl]benzoyl]-3,9-diazaspiro[5.5]undecan-9-yl]-2-fluoro-phenyl]piperidine-2,6-dione (Compound 228)

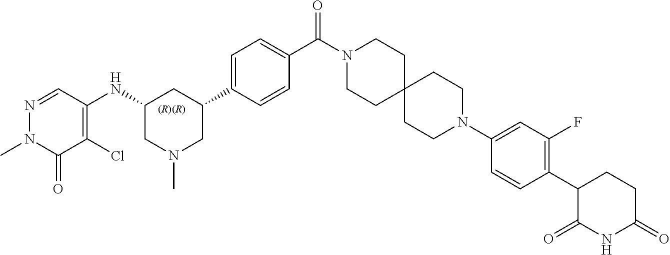

Compound 228 was prepared by general procedure 1. Intermediate 8 was used in the final step to afford 3-[4-[3-[4-[(3R,5R)-5-[(5-chloro-1-methyl-6-oxo-pyridazin-4-yl)amino]-1-methyl-3-piperidyl]benzoyl]-3,9-diazaspiro[5.5]undecan-9-yl]-2-fluoro-phenyl]piperidine-2,6-dione (0.07 g, 17%) as an off white solid. ¹H NMR (400 MHz, DMSO-d₆) δ=10.78 (s, 1H), 7.96 (s, 1H), 7.37-7.29 (m, 4H), 7.06 (t, J=8.8 Hz, 1H), 6.71 (d, J=11.8 Hz, 2H), 6.10 (d, J=9.0 Hz, 1H), 4.00-3.92 (m, 1H), 3.87 (dd, J=4.9, 12.3 Hz, 1H), 3.71-3.49 (m, 5H), 3.18 (brs, 4H), 3.05-2.91 (m, 2H), 2.86 (dd, J=3.0, 10.9 Hz, 1H), 2.75-2.65 (m, 1H), 2.25 (s, 3H), 2.13 (dd, J=4.0, 12.5 Hz, 1H), 2.06-2.05 (m, 1H), 2.01 (d, J=6.0 Hz, 1H), 1.99-1.88 (m, 3H), 1.69 (q, J=12.1 Hz, 1H), 1.58 (brs, 4H), 1.54-1.39 (m, 4H), 2H merged in solvent peak. LC-MS: m/z 718.40 [M+H]⁺.

### Example 10: 3-[4-[3-[4-[(3R,5R)-5-[(1,5-dimethyl-6-oxo-pyridazin-4-yl)amino]-1-methyl-3-piperidyl]benzoyl]-3,9-diazaspiro[5.5]undecan-9-yl]-2-fluoro-phenyl]piperidine-2,6-dione (Compound 229)

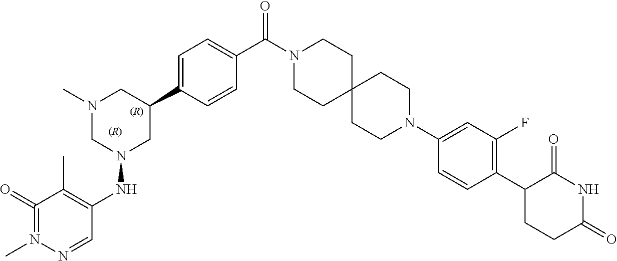

Compound 229 was prepared by general procedure 1. Intermediate 17 was used in the final step to afford 3-[4-[3-[4-[(3R,5R)-5-[(1,5-dimethyl-6-oxo-pyridazin-4-yl)amino]-1-methyl-3-piperidyl]benzoyl]-3,9-diazaspiro[5.5]undecan-9-yl]-2-fluoro-phenyl]piperidine-2,6-dione (0.04 g, 11%) as an off white solid. ¹H NMR (400 MHz, DMSO-d₆) δ=10.78 (s, 1H), 7.78 (s, 1H), 7.36-7.30 (m, 4H), 7.06 (t, J=: 8.8 Hz, 1H), 6.71 (d, J=11.8 Hz, 2H), 5.47 (d, J=8.9 Hz, 1H), 3.90-3.79 (m, 2H), 3.62 (brs, 2H), 3.52 (s, 3H), 3.21-3.13 (m, 4H), 3.03-2.93 (m, 2H), 2.87 (dd, J=2.7, 10.7 Hz, 1H), 2.76-2.66 (m, 1H), 2.50-2.47 (m, 4H), 2.24 (s, 3H), 2.13 (dd, J=4.1, 12.7 Hz, 1H), 2.04 (d, J=12.0 Hz, 1H), 1.98-1.87 (m, 3H), 1.84 (s, 3H), 1.62-1.55 (m, 4H), 1.54-1.40 (m, 4H). LC-MS: m/z 698.77 [M+H]⁺.

### Example 11. Preparation of Additional Compounds

In some embodiments, the present disclosure provides technologies useful for, among other things, manufacturing various compounds described herein. Certain useful technologies are described herein as examples.

For example, in some embodiments, the present disclosure provides a compound having the structure of

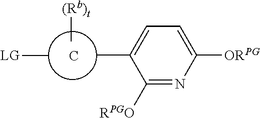

or a salt thereof, wherein LG is a leaving group, R^{PG} is a protecting group, and the other variables are as defined and described in classes and subclasses herein, both singly and in combination. Various leaving groups are available and can be utilized in accordance with the present disclosure. For example, in some embodiments, a leaving group utilized in the present disclosure is —Cl, —Br, —I, —OTs, or —OTf. Various protecting groups, e.g., for —OH, are available and can be utilized in accordance with the present disclosure. For example, in some embodiments, R^{PG} is Bn. In some embodiments, Ring C is Ring Cy^{a}, wherein Cy^{a} is Cy^{a} is optionally substituted mono- or bicyclic 6- to 10-membered aryl or optionally substituted mono- or bicyclic 5- to 10-membered heteroaryl having 1-4 heteroatoms independently selected from N, O and S. In some embodiments, the present disclosure provides a compound having the structure of

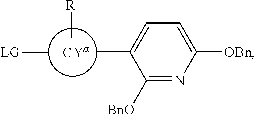

wherein LG is —Cl, —Br or —I, and Cy^{a} is optionally substituted mono- or bicyclic 6- to 10-membered aryl or optionally substituted mono- or bicyclic 5- to 10-membered heteroaryl having 1-4 heteroatoms independently selected from N, O and S.

### General Intermediate 21

### Scheme for General Intermediate 21

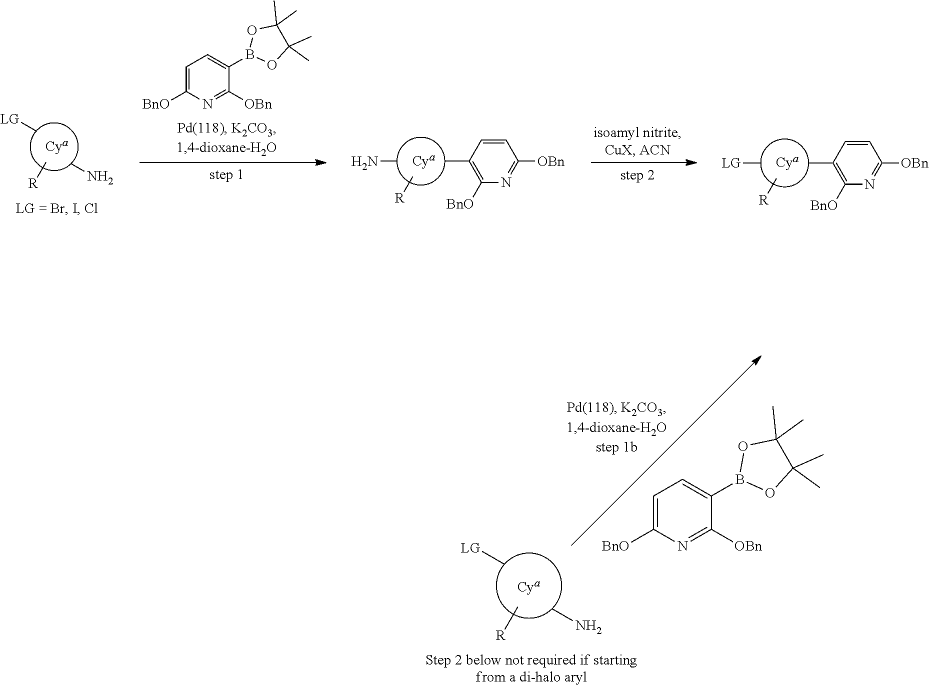

### Step 1 & Step 1b

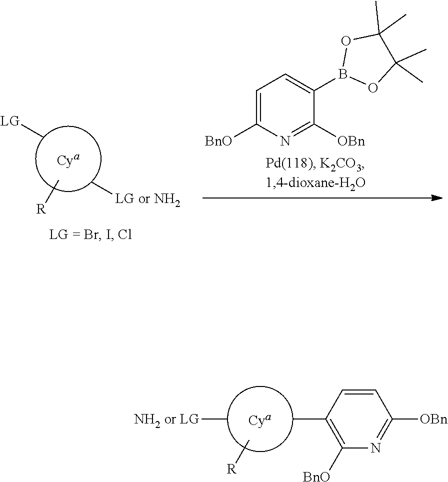

To a stirred solution of substituted halo-aniline or dihaloaryl (13.00 mmol) in 1,4-dioxane (2 mL per mmol) is added 2,6-dibenzyloxy-3-(4,4,5,5-tetramethyl-1,3,2-dioxaborolan-2-yl)pyridine (15.00 mmol) followed by potassium carbonate (25.00 mmol) dissolved in water and the reaction mixture is purged with argon for 30 min. To the resulting reaction mixture, Pd-118 (0.83 g, 1.30 mmol) is added and the reaction mixture is again purged with argon for 20 min. The reaction mixture is stirred in a sealed tube at 100° C. for 16 h. The progress of the reaction is monitored by TLC. After completion of reaction, the reaction mixture is filtered through celite. The filtrate is diluted with cold water and extracted with ethyl acetate. The combined organic layer is washed with water and brine, dried over anhydrous Na₂SO₄ and concentrated under reduced pressure. The crude compound is purified by silica gel column chromatography (50% EtOAc in heptane) to afford desired intermediate. Follow step 2 if aniline starting material used in step 1.

### Step 2

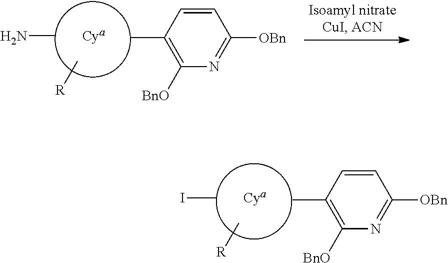

To a stirred solution of intermediate from step 1 (4.80 mmol) in ACN (8 mL per mmol) is added isoamyl nitrite (29.00 mmol) at RT. Stir the reaction for 10 min then add copper (I) iodide (14.00 mmol) at RT. The reaction mixture is stirred at 60° C. for 1 h. The progress of reaction is monitored by TLC and LCMS. After completion of reaction, the reaction mixture is diluted with cold water and extracted with ethyl acetate. The combined organic layer is washed with water and brine, dried over anhydrous Na₂SO₄ and concentrated under reduced pressure. The crude compound is purified by silica-gel column chromatography (5% EtOAc in heptane) to afford desired intermediate.

### General Intermediate 22

### Scheme for General Intermediate 22

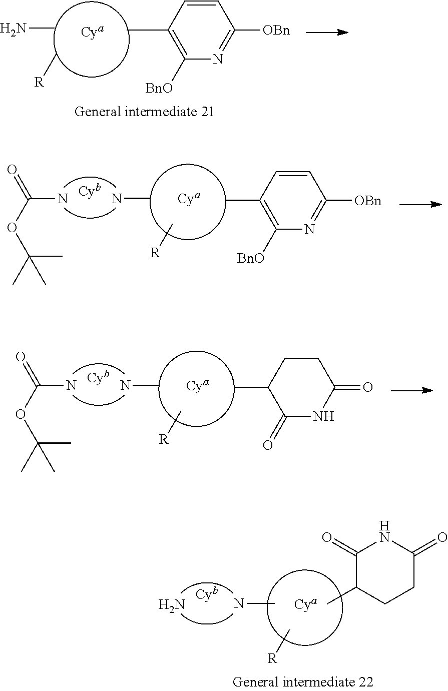

Cy^{b}=optionally substituted mono-, bi-, or spirocyclic 5- to 13-membered ring

### Step 1

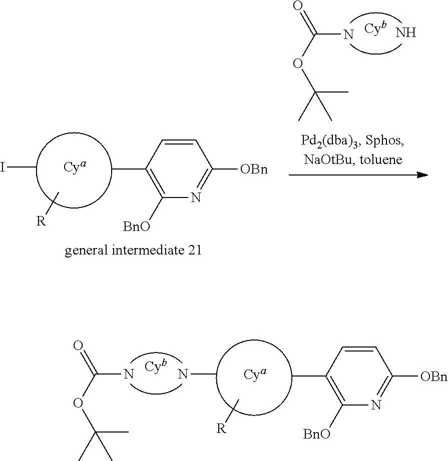

To a stirred solution of general intermediate 21 (2.30 mmol) in toluene (9 mL per mmol) is added boc-diamine (2.70 mmol) followed by sodium tert-butoxide (3.40 mmol) at RT. The reaction mixture is purged with argon for 30 min. To the resulting reaction mixture, 2-dicyclohexylphosphino-2′,6′-dimethoxybiphenyl (0.09 g, 0.23 mmol) and tris(dibenzylideneacetone)dipalladium(0) (0.11 mmol) is added at RT and the reaction mixture is again purged with argon for 20 min. The reaction mixture is stirred in a sealed tube at 110° C. for 16 h. The progress of the reaction is monitored by TLC. After completion of reaction, the reaction mixture is filtered through celite. The filtrate is diluted with cold water and extracted with ethyl acetate. The combined organic layer is washed with water and brine, dried over anhydrous Na₂SO₄ and concentrated under reduced pressure. The crude compound is purified by silica-gel column chromatography (50% EtOAc in heptane) to afford the desired product.

### Step 2

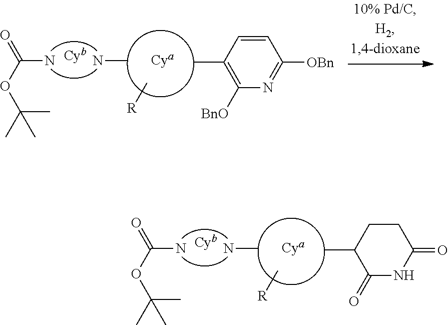

To a stirred solution of intermediate from step 1 (0.69 mmol) in 1,4-dioxane (14.5 mL per mmol) is added 10% Pd/C (0.30 g) at RT. The reaction mixture is stirred in a steel bomb under 50 psi H₂ gas pressure at RT for 16 h. The progress of the reaction is monitored by TLC and LCMS. After completion of reaction, the reaction mixture is filtered through celite. The filtrate is concentrated under reduced pressure. The crude compound is purified by silica-gel column chromatography (5% MeOH in DCM) to afford the desired intermediate.

### Step 3

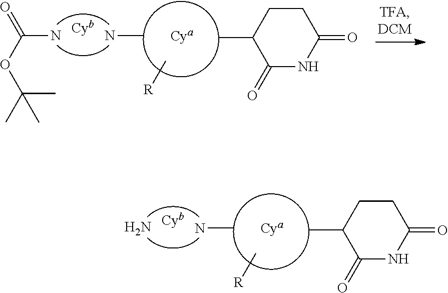

To a stirred solution of intermediate from step 2 (0.49 mmol, from step 2) in DCM (10 mL) is added TFA (2 mL) at 0° C. and the reaction mixture is stirred at 0° C. for 5 min followed by at RT for 3 h. The progress of the reaction is monitored by TLC. After completion of reaction, the reaction mixture is concentrated under reduced pressure to afford the desired product.

### General Intermediate 23: 4-[(3R,5R)-5-[(5-bromo-1-methyl-6-oxo-pyridazin-4-yl)amino]-1-methyl-3-piperidyl]benzoic acid

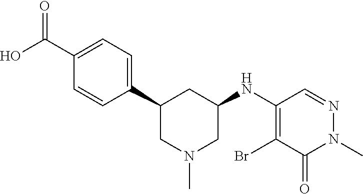 [SMILES: CN1C[C@H](Nc2cnn(C)c(=O)c2Br)C[C@H](c2ccc(C(=O)O)cc2)C1]

### Step 1: tert-butyl (3R)-3-(pyridine-2-carbonylamino)piperidine-1-carboxylate

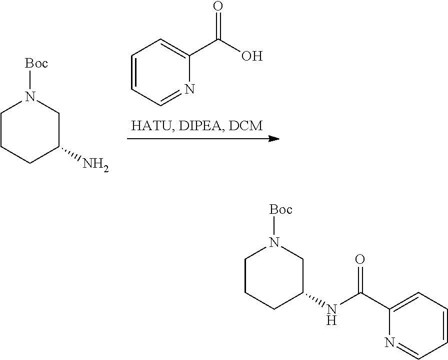 [SMILES: CC(C)(C)OC(=O)N1CCC[C@@H](N)C1 ; O=C(O)c1ccccn1 ; CC(C)(C)OC(=O)N1CCC[C@@H](NC(=O)c2ccccn2)C1]

To a stirred solution of picolinic acid (10 g, 50.00 mmol), tert-butyl (R)-3-aminopiperidine-1-carboxylate (7.40 g, 60.00 mmol) and DIPEA (13 mL, 75.00 mmol) in DCM (100 mL) is added HATU (23 g, 60.00 mmol) and the reaction mixture is stirred at ambient temperature for 16 h. The progress of the reaction is monitored by TLC. After completion of reaction, the reaction mixture is concentrated under reduced pressure. The residue is dissolved in ethyl acetate and washed with 0.05 M HCl and saturated NaHCO₃ solution. The organic layer is dried over anhydrous Na₂SO₄ and concentrated under reduced pressure. The crude compound is purified by silica-gel column chromatography to afford tert-butyl (3R)-3-(pyridine-2-carbonylamino)piperidine-1-carboxylate (13.17 g, 72%) as a white solid. ¹H NMR (400 MHz, DMSO-d₆) δ=8.63 (d, J=3.9 Hz, 1H), 8.55 (brs, 1H), 8.07-7.98 (m, 2H), 7.61 (t, J=5.6 Hz, 1H), 3.90-3.79 (m, 2H), 3.76-3.49 (m, 2H), 2.99 (brs, 2H), 1.82 (brs, 1H), 1.74-1.61 (m, 2H), 1.37 (brs, 9H); LC-MS: m/z 306.2 [M+H]⁺.

### Step 2: tert-butyl (3R,5R)-3-(4-methoxycarbonylphenyl)-5-(pyridine-2-carbonylamino)piperidine-1-carboxylate

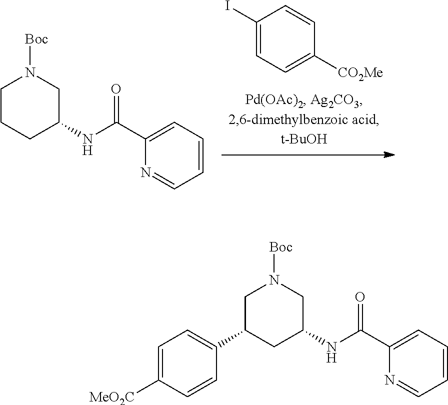 [SMILES: CC(C)(C)OC(=O)N1CCC[C@@H](NC(=O)c2ccccn2)C1 ; COC(=O)c1ccc(I)cc1 ; COC(=O)c1ccc([C@H]2C[C@@H](NC(=O)c3ccccn3)CN(C(=O)OC(C)(C)C)C2)cc1]

A pressure vial (100 mL) equipped with a magnetic stirring bar is charged with tert-butyl (3R)-3-(pyridine-2-carbonylamino)piperidine-1-carboxylate (10.00 g, 32.8 mmol, from step 1), silver carbonate (9 g, 32.8 mmol), Pd(OAc)₂ (0.74 g, 0.32 mmol), methyl 4-iodobenzoate (43 g, 164 mmol), 2,6-dimethylbenzoic acid (1.23 g, 8.2 mmol) and t-BuOH (100 mL). The vessel is flushed with argon, scaled with a crimp cap and is heated to 120° C. After 24 h, the reaction vessel is removed from the oil bath and cooled to room temperature and DCM is added to the reaction mixture. The progress of the reaction is monitored by TLC. The mixture is thoroughly stirred for 10 min and the solids are removed by filtration which is additionally rinsed with DCM. The combined filtrates are concentrated under reduced pressure and the residue is purified by silica-gel column chromatography to afford tort-butyl (3R,5R)-3-(4-methoxycarbonylphenyl)-5-(pyridine-2-carbonylamino)piperidine-1-carboxylate (8.00 g, 56%) as a white solid. LC-MS: m/z 438.12 [M−H]⁻.

### Step 3: Methyl 4-[(3R,5R)-3-(pyridine-2-carbonylamino)-3-piperidyl]benzoate

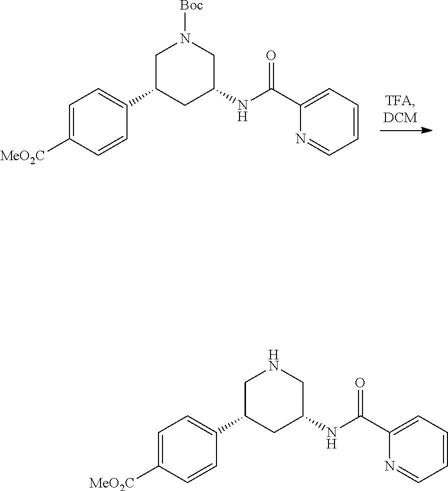 [SMILES: COC(=O)c1ccc([C@H]2C[C@@H](NC(=O)c3ccccn3)CN(C(=O)OC(C)(C)C)C2)cc1 ; COC(=O)c1ccc([C@@H]2CNC[C@H](NC(=O)c3ccccn3)C2)cc1]

To a stirring solution of tert-butyl (3R,5R)-3-(4-methoxycarbonylphenyl)-5-(pyridine-2-carbonylamino)piperidine-1-carboxylate (6.00 g, 13.66 mmol, from step 2) in DCM (70 mL) is added TFA (12 mL) at 0° C. Then the resulting solution is slowly brought to room temperature and stirred for 7 h. The progress of the reaction is monitored by TLC. The solvent is removed under reduced pressure, and the crude product is redissolved in water and extracted with ethyl acetate. The combined organic layer is dried over anhydrous Na₂SO₄, filtered, and concentrated in vacuo to afford methyl 4-[(3R,5R)-5-(pyridine-2-carbonylamino)-3-piperidyl]benzoate (2.7 g, 58%) as a pale-yellow solid which is used for the next reaction without any further purification. ¹H NMR (400 MHz, DMSO-d₆) δ=8.65-8.63 (m, 1H), 8.52 (d, J=8.6 Hz, 1H), 8.05-7.97 (m, 2H), 7.92-7.89 (m, 2H), 7.62-7.58 (m, 1H), 7.41 (d, J=8.3 Hz, 2H), 4.06-4.00 (m, 1H), 3.84 (s, 3H), 3.08-2.95 (m, 3H), 2.91-2.85 (m, 1H), 2.55 (s, 1H), 2.45 (s, 1H), 2.04 (d, J=12.2 Hz, 1H), 1.93-1.85 (m, 1H); LC-MS: m/z 340.22 [M+H]⁺.

### Step 4: methyl 4-[(3R,5R)-1-methyl-5-(pyridine-2-carbonylamino)-3-piperidyl]benzoate

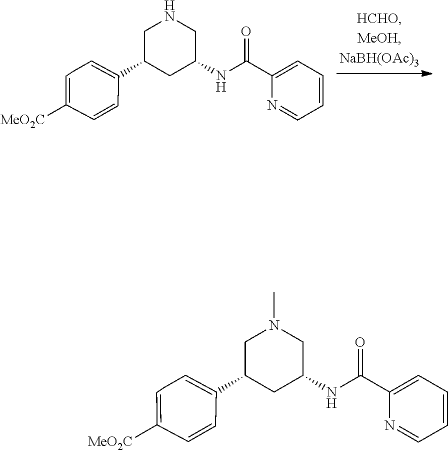 [SMILES: COC(=O)C1=CCC=C([C@@H]2CNC[C@H](NC(=O)c3ccccn3)C2)C=C1 ; COC(=O)C1=CCC=C([C@H]2C[C@@H](NC(=O)c3ccccn3)CN(C)C2)C=C1]

To a stirred solution of methyl 4-[(3R,5R)-5-(pyridine-2-carbonylamino)-3-piperidyl]benzoate (7.0 g, 21 mmol, from step 3) and glacial acetic acid (1 mL) in methanol (70 mL) is added 37% w/v formaldehyde (2.9 mL) and stirred for 5 h. Then sodium triacetoxyborohydride (6.53 g, 31 mmol) is added over a period of 30 min at 0° C. The resulting solution is stirred for 16 h at RT. The progress of the reaction is monitored by TLC. After completion of reaction, the solvent is removed in vacuo and the residue is partitioned between ethyl acetate and water. The biphasic solution is extracted twice with ethyl acetate. The combined organic layer is washed with brine, dried over anhydrous Na₂SO₄, filtered and concentrated. Crude material is purified by silica-gel column chromatography to afford methyl 4-[(3R,5R)-1-methyl-5-(pyridine-2-carbonylamino)-3-piperidyl]benzoate (5.5 g, 75%). ¹H NMR (400 MHz, DMSO-d=) δ=8.67-8.53 (m, 2H), 8.08-7.95 (m, 2H), 7.91 (d, J=8.3 Hz, 2H), 7.63-7.55 (m, 1H), 7.44 (d, J=8.3 Hz, 2H), 4.21-4.06 (m, 1H), 3.84 (s, 3H), 3.09-2.90 (m, 2H), 2.85 (d, J=9.8 Hz, 1H), 2.25 (s, 3H), 2.04-1.89 (m, 3H), 1.84-1.69 (m, 1H); LC-MS: m/z 354.12 [M+H]⁺.

### Step-5: methyl 4-((3R,5R)-5-amino-1-methylpiperidin-3-yl)benzoate

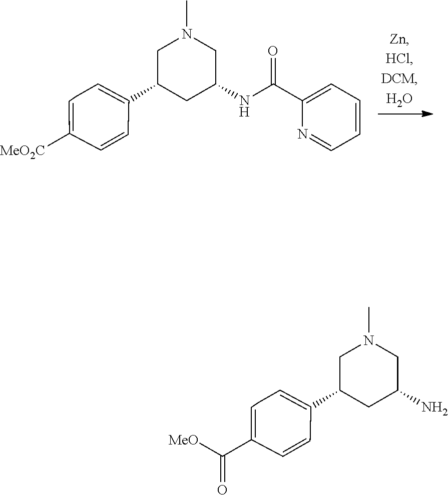 [SMILES: COC(=O)C1=CCC=C([C@H]2C[C@@H](NC(=O)c3ccccn3)CN(C)C2)C=C1 ; COC(=O)C1=CCC=C([C@H]2C[C@@H](N)CN(C)C2)C=C1]

To a stirred suspension of methyl 4-[(3R,5R)-1-methyl-5-(pyridine-2-carbonylamino)-3-piperidyl]benzoate (1.00 g, 2.80 mmol, from step 4) in water (50 mL) is added 12 N HCl solution (5 mL) at 0° C., followed by zinc powder (2.76 g, 42.00 mmol) and DCM (50 mL) and the reaction mixture is stirred at RT for 12 h in a round bottomed flask. The progress of the reaction is monitored by TLC. After completion of the reaction, the reaction is cooled to 0° C. and basified using NaOH (5M) until pH 9. Then the mixture is filtered through a Celite® bed and washed with DCM (20 mL×3). The filtrate is extracted with DCM (20 mL×3), all organic layers are combined, and solvent is removed in vacuo. Crude material (700 mg) is purified by silica-gel column chromatography (DCM:MeOH:NH₄OH 80:20:03) to afford methyl 4-((3R,5R)-5-amino-1-methylpiperidin-3-yl)benzoate (0.45 g, 64%) as an off white solid. LC-MS: m/z 249.37 [M+H]⁺.

### Step 6: methyl 4-((3R,5R)-5-((5-bromo-1-methyl-6-oxo-1,6-dihydropyridazin-4-yl)amino)-1-methylpiperidin-3-yl)benzoate

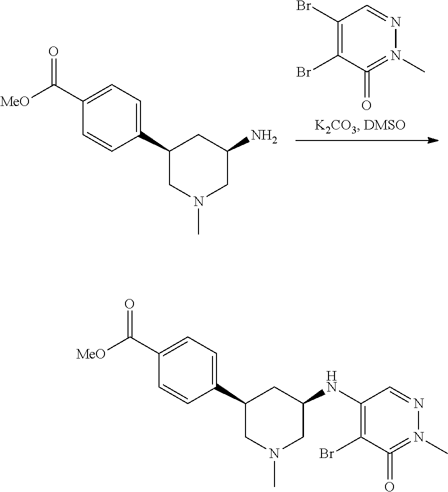

To a stirred solution of methyl 4-((cis)-5-amino-1-methylpiperidin-3-yl)benzoate (3.00 g, 12.09 mmol, from step 5) in DMSO (30 mL), K₂CO₃ (5.00 g, 36.29 mmol) is added, followed by 4,5-dibromo-2-methylpyridazin-3(2H)-one (general intermediate 29, 4.86 g, 18.14 mmol), and the reaction mixture is stirred at 100° C. for 16 h. Progress of reaction is monitored by TLC and LCMS. After completion of reaction, the reaction mixture is dissolved in water and extracted with ethyl acetate. The combined organic layer is dried over anhydrous Na₂SO₄ and concentrated under reduced pressure. The crude compound is purified by silica-gel column chromatography (2% methanol in DCM) to afford methyl 4-((3R,5R)-5-((5-bromo-1-methyl-6-oxo-1,6-dihydropyridazin-4-yl)amino)-1-methylpiperidin-3-yl)benzoate: ¹H NMR (400 MHz, DMSO-d₆) δ=7.95-7.85 (m, 3H), 7.42 (d, J=8.3 Hz, 2H), 5.82 (d, J=9.3 Hz, 1H), 4.02-3.89 (m, 1H), 3.84 (s, 3H), 3.59 (s, 3H), 3.05 (t, J=11.5 Hz, 1H), 2.95 (d, J=6.8 Hz, 1H), 2.90-2.80 (m, 1H), 2.25 (s, 3H), 2.09-1.87 (m, 3H), 1.80-1.66 (m, 1H). LC-MS: m/z 435.1 [M+H]⁺.

### Step 7: 4-((3R,5R)-5-((5-bromo-1-methyl-6-oxo-1,6-dihydropyridazin-4-yl)amino)-1-methylpiperidin-3-yl)benzoic acid

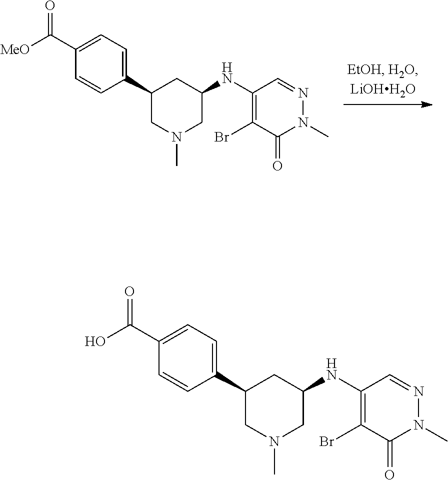 [SMILES: COC(=O)c1ccc([C@H]2C[C@@H](Nc3cnn(C)c(=O)c3Br)CN(C)C2)cc1 ; CN1C[C@H](Nc2cnn(C)c(=O)c2Br)C[C@H](c2ccc(C(=O)O)cc2)C1]

To a stirred solution of methyl 4-((3R,5R)-5-((5-bromo-1-methyl-6-oxo-1,6-dihydropyridazin-4-yl)amino)-1-methylpiperidin-3-yl)benzoate (0.23 g, 0.52 mmol, from step 6) in ethanol (5 mL) and water (0.5 mL), lithium hydroxide (0.03 g, 0.63 mmol) is added and the reaction mixture is stirred at 70° C. for 2 h. The progress of the reaction is monitored by TLC. The reaction mixture is concentrated under reduced pressure. The crude is acidified with 1N HCl to pH 3. Precipitated solid is filtered, washed and concentrated to yield 4-((3R,5R)-5-((5-bromo-1-methyl-6-oxo-1,6-dihydropyridazin-4-yl)amino)-1-methylpiperidin-3-yl)benzoic acid (0.20 g, 91%) as an off white solid. ¹H NMR (400 MHz, DMSO-d₆) δ=13.13-12.82 (m, 1H), 11.34-11.09 (m, 1H), 7.96 (d, J=6.8 Hz, 3H), 7.43 (d, J=8.3 Hz, 2H), 6.29 (d, J=8.3 Hz, 1H), 4.53-4.36 (m, 1H), 3.62 (s, 3H), 3.57-3.47 (m, 1H), 3.46-3.36 (m, 1H), 3.15-3.03 (m, 1H), 2.93 (d, J=8.8 Hz, 1H), 2.80 (s, 3H), 2.18 (d, J=10.3 Hz, 1H), 2.12-2.01 (m, 1H). LC-MS: m/z 422.0 [M+H]⁺.

### General Intermediate 24: 4-[(3R,5R)-5-[(5-chloro-1-methyl-6-oxo-pyridazin-4-yl)amino]-1-methyl-3-piperidyl]benzoic acid

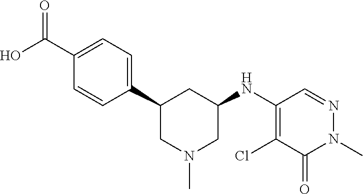 [SMILES: CN1C[C@H](Nc2cnn(C)c(=O)c2Cl)C[C@H](c2ccc(C(=O)O)cc2)C1]

### Step 1: ethyl 4-[(3R,5R)-5-[(5-chloro-1-methyl-6-oxo-pyridazin-4-yl)amino]-1-methyl-3-piperidyl]benzoate

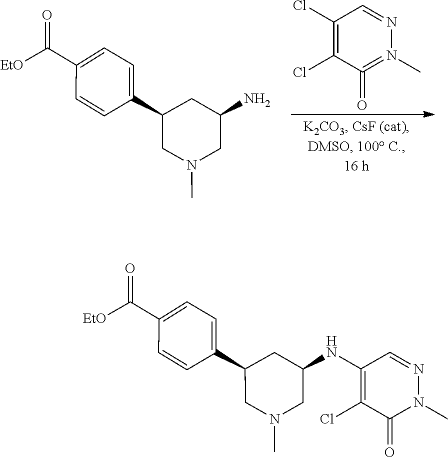

To a stirred solution of ethyl 4-[(3R,5R)-5-amino-1-methyl-3-piperidyl]benzoate (12.00 g, 48.33 mmol, from step 5 of general intermediate 23) and 4,5-dichloro-2-methyl-pyridazin-3-one (8.91 g, 48.33 mmol) in DMSO (60 mL) are added K₂CO₃ (20.00 g, 144.00 mmol) and CsF (0.74 g, 4.83 mmol), respectively, at RT. The reaction mixture is stirred at 100° C. for 16 h. The progress of the reaction is monitored by TLC. After completion of reaction, the reaction mixture is diluted with cold water and extracted with ethyl acetate. The combined organic layer is washed with water and brine, respectively, then dried over anhydrous Na₂SO₄, filtered and concentrated under reduced pressure to afford ethyl 4-[(3R,5R)-5-[(5-chloro-1-methyl-6-oxo-pyridazin-4-yl)amino]-1-methyl-3-piperidyl]benzoate (5.50 g, 29%) as an off white solid. ¹H NMR (400 MHz, DMSO-d₆) δ=7.97 (s, 1H), 7.92 (d, J=8.3 Hz, 2H), 7.41 (d, J=8.1 Hz, 2H), 6.16 (d, J=9.2 Hz, 1H), 4.30 (q, J=7.1 Hz, 2H), 3.98-3.94 (m, 1H), 3.58 (s, 3H), 3.04 (t, J=11.7 Hz, 1H), 2.97-2.84 (m, 2H), 2.25 (s, 3H), 2.07-1.95 (m, 2H), 1.91 (t, J=11.1 Hz, 1H), 1.76-1.64 (m, 1H), 1.31 (t, J=7.0 Hz, 3H); LC-MS: m/z 405.40 [M+H]⁺.

### Step 2: 4-[(3R,5R)-5-[(5-chloro-1-methyl-6-oxo-pyridazin-4-yl)amino]-1-methyl-3-piperidyl]benzoic acid

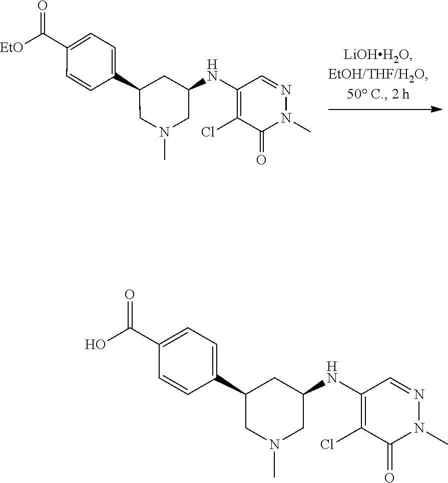 [SMILES: CCOC(=O)c1ccc([C@H]2C[C@@H](Nc3cnn(C)c(=O)c3Cl)CN(C)C2)cc1 ; CN1C[C@H](Nc2cnn(C)c(=O)c2Cl)C[C@H](c2ccc(C(=O)O)cc2)C1]

To a stirred solution of ethyl 4-[(3R,5R)-5-[(5-chloro-1-methyl-6-oxo-pyridazin-4-yl)amino]-1-methyl-3-piperidyl]benzoate (3.00 g, 7.40 mmol from step 1) in ethanol (10 mL), THF (10 mL) and H₂O (10 mL) is added LiOH·H₂O (0.35 g, 14.82 mmol) at RT. The reaction mixture is stirred at 50° C. for 2 h. After completion of reaction, the reaction mixture is concentrated, and washed with diethyl ether to obtain crude. The crude is diluted with water, acidified with 1N HCl (pH 3) to get solid, which is filtered and washed with diethyl ether to afford 4-[(3R,5R)-5-[(5-chloro-1-methyl-6-oxo-pyridazin-4-yl)amino]-1-methyl-3-piperidyl]benzoic acid (2.10 g, 75%) as an off white solid. LC-MS: m/z 377.20 [M+H]⁺.

### General Intermediate 25: 4-[(3R,5R)-5-[(1,5-dimethyl-6-oxo-pyridazin-4-yl)amino]-1-methyl-3-piperidyl]benzoic acid

### Step 1: ethyl 4-[(3R,5R)-5-[(1,5-dimethyl-6-oxo-pyridazin-4-yl)amino]-1-methyl-3-piperidyl]benzoate

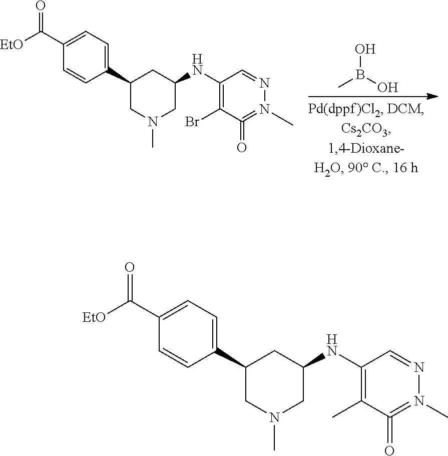

To a stirred solution of ethyl 4-[(3R,5R)-5-[(5-bromo-1-methyl-6-oxo-pyridazin-4-yl)amino]-1-methyl-3-piperidyl]benzoate (5.00 g, 11.13 mmol, from step 6 intermediate 3) in 1,4-dioxane (49 mL) and H₂O (1 mL) is added methylboronic acid (1.33 g, 22.25 mmol) followed by Cs₂CO₃ (10.90 g, 33.30 mmol) at RT. The reaction mixture is purged with argon for 30 min. To the resulting reaction mixture, PdCl₂(dppf).DCM (0.85 g, 1.11 mmol) is added and the reaction mixture is again purged with argon for 20 min. The reaction mixture is stirred in a sealed tube at 90° C. for 16 h. The progress of the reaction is monitored by TLC. After completion of reaction, the reaction mixture is filtered through a pad of celite. The filtrate is diluted with cold water and extracted with ethyl acetate. The combined organic layer is washed with water and brine, respectively, dried over anhydrous Na₂SO₄ and concentrated under reduced pressure to obtain crude. The crude compound is purified by silica gel column chromatography (10% MeOH in DCM) to afford ethyl 4-[(3R,5R)-5-[(1,5-dimethyl-6-oxo-pyridazin-4-yl)amino]-1-methyl-3-piperidyl]benzoate (2.80 g, 65%) as a brown solid. LC-MS: m/z 385.20 [M+H]⁺.

### Step 2: 4-[(3R,5R)-5-[(1,5-dimethyl-6-oxo-pyridazin-4-yl)amino]-1-methyl-3-piperidyl]benzoic acid

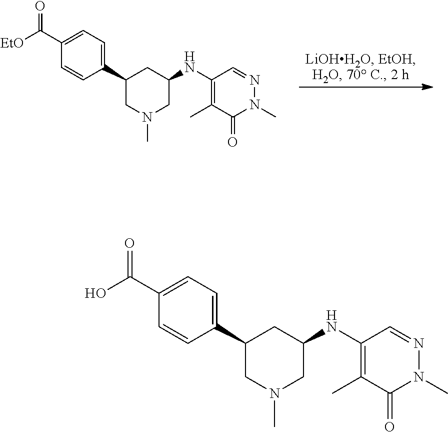 [SMILES: CCOC(=O)c1ccc([C@H]2C[C@@H](Nc3cnn(C)c(=O)c3C)CN(C)C2)cc1 ; "LiOH·H2O, EtOH,H2O, 70° C., 2 h" ; Cc1c(N[C@@H]2C[C@H](c3ccc(C(=O)O)cc3)CN(C)C2)cnn(C)c1=O]

To a stirred solution of ethyl 4-[(3R,5R)-5-[(1,5-dimethyl-6-oxo-pyridazin-4-yl)amino]-1-methyl-3-piperidyl]benzoate (2.50 g, 6.20 mmol) in ethanol (20 mL) and H₂O (4 mL) is added LiOH·H₂O (0.54 g, 22.54 mmol) at RT. The reaction mixture is stirred at 70° C. for 2 h. The progress of the reaction is monitored by TLC. After completion of reaction, the reaction mixture is concentrated, and the residue is washed with diethyl ether to obtain crude. The crude is diluted with water and acidified with 1N aq. HCl (pH 3) to get solid, which is filtered and washed with diethyl ether to afford 4-[(3R,5R)-5-[(1,5-dimethyl-6-oxo-pyridazin-4-yl)amino]-1-methyl-3-piperidyl]benzoic acid (2.10 g, 90%) as brown solid. ¹H NMR (400 MHz, DMSO-d₆) δ=7.89 (d, J=7.8 Hz, 2H), 7.79 (s, 1H), 7.34 (d, J=7.8 Hz, 2H), 5.59-5.47 (m, 1H), 3.91-3.79 (m, 2H), 3.53 (s, 3H), 3.08-2.82 (m, 3H), 2.26 (s, 3H), 2.04 (d, J=11.7 Hz, 1H), 1.92 (t, J=10.8 Hz, 2H), 1.86 (s, 3H), —COOH proton is absent. LC-MS: m/z 356.90 [M+H]⁺.

### General Intermediate 26-28

### Step 1

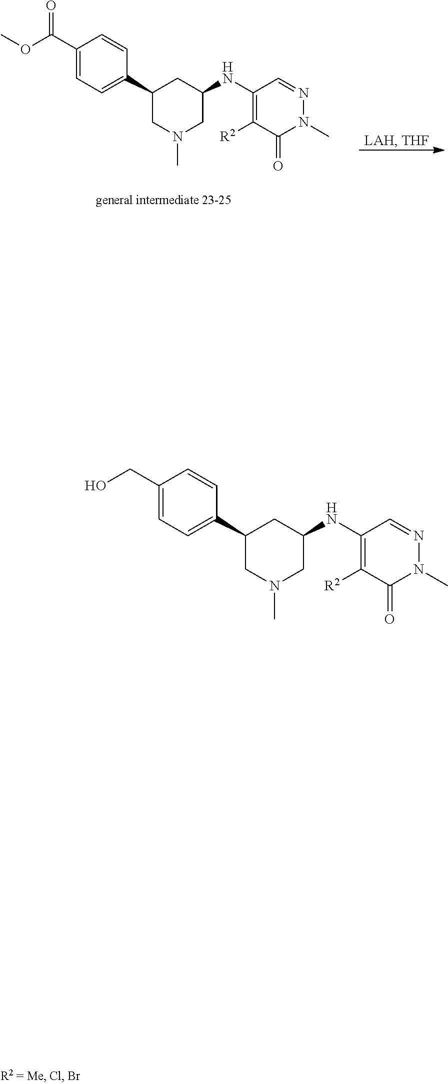

To a cooled stirred solution of general intermediate 23-25 (2.87 mmol) in THF (25 mL), is added LAH (2M in THF, 4.59 mmol) at 0° C. The reaction mixture is allowed to warm to RT and stirred for 4 h. The progress of the reaction is monitored by TLC. After completion of reaction, the reaction mixture is quenched with saturated Na₂SO₄ solution and diluted with water and extracted with ethyl acetate. The combined organic layer is dried over anhydrous Na₂SO₄ and concentrated under reduced pressure to afford crude compound which is purified by silica-gel column chromatography (80% ethyl acetate in hexane) to desired intermediate.

### Step 2

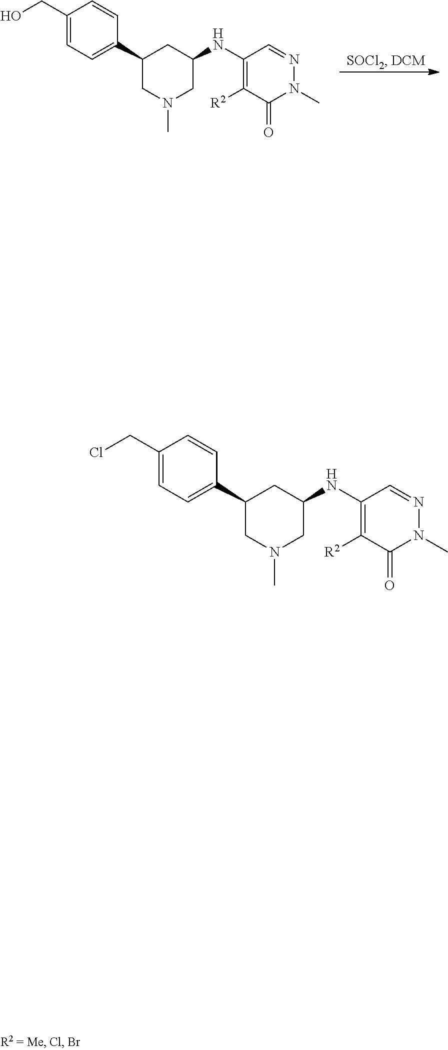

To a stirred solution of intermediate from step 1 (0.19 mmol, step 1 of intermediate 24) in DCM (10 mL), thionyl chloride (0.39 mmol) is added at 0° C., and the reaction mixture is stirred at RT for 4 h. The progress of the reaction is monitored by TLC. After completion of reaction, the reaction mixture is concentrated under reduced pressure. The residue is diluted with water, and the aqueous layer is extracted with ethyl acetate. The combined organic layer is dried over anhydrous Na₂SO₄ and concentrated under reduced pressure to afford crude compound which is purified by silica-gel column chromatography (50% ethyl acetate in hexane) to afford the desired general intermediate.

### General Intermediate 29: 4,5-dibromo-2-methylpyridazin-3(2H)-one

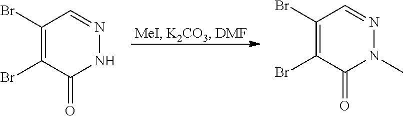

To a stirred solution of 4,5-dibromopyridazin-3 (2H)-one (20.00 g, 79.00 mmol) in DMF (200 mL), K₂CO₃ (21.00 g, 158.10 mmol) is added, followed by methyl iodide (8.80 mL, 118.10 mmol), and the reaction mixture is stirred at RT for 16 h. The progress of the reaction is monitored by TLC. After completion of reaction, the reaction mixture is quenched with water. The aqueous layer is extracted with ethyl acetate. The combined organic layer is washed with brine, dried over anhydrous Na₂SO₄ and concentrated under reduced pressure. The crude compound is washed with diethyl ether to afford 4,5-dibromo-2-methylpyridazin-3(2H)-one (14.00 g, 66.8%) as colorless oil which is used as such for the next reaction. ¹H NMR (400 MHz, DMSO-d₆) δ=8.12 (s, 1H), 3.67 (s, 3H). LC-MS: m/z 268.7 [M+H]⁺.

### General Procedure 4

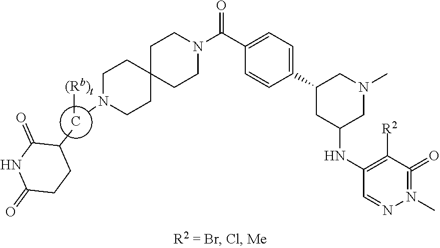

In various embodiments,

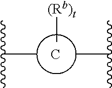

is

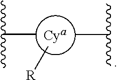

### Step-1

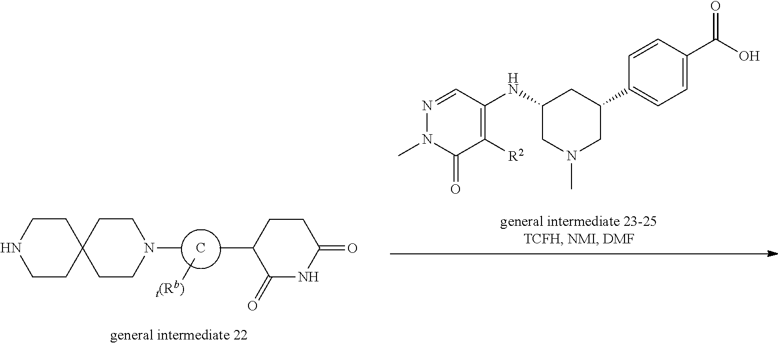

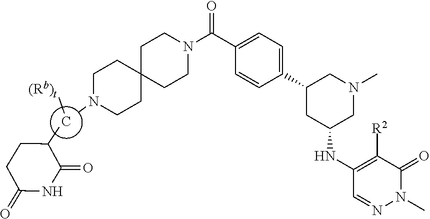

To a stirred solution of general intermediate 22 (0.30 mmol) in DMF (4 mL) is added 1-methylimidazole (1.00 mmol) followed by general intermediate 23-25 (0.30 mmol) and the reaction mixture is stirred at RT for 10 min. To this is added N,N,N′,N′-tetramethylchloroformamidinium hexafluorophosphate (0.40 mmol) and the reaction mixture is stirred at 80° C. for 16 h. The progress of the reaction is monitored by TLC. After completion of reaction, the reaction mixture is diluted with ice-cold water and extracted with ethyl acetate. The combined organic layer is dried over anhydrous Na₂SO₄ and concentrated under reduced pressure, the crude compound is purified by prep HPLC to give the desired product.

### General Procedure 5

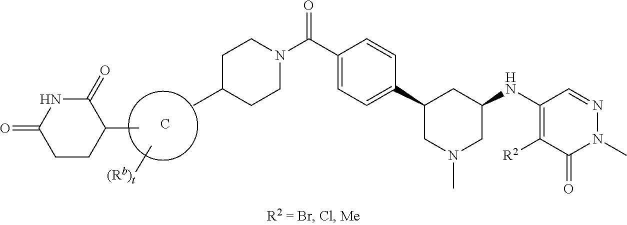

### Step-1

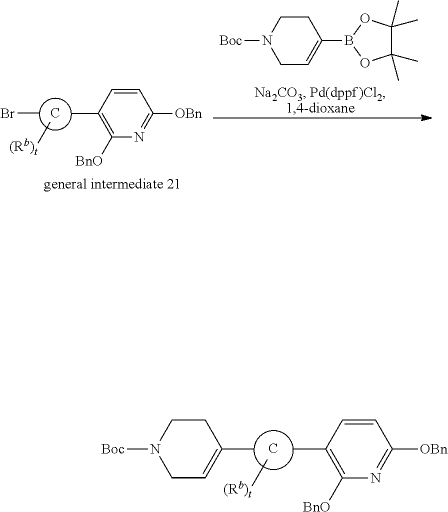

To a stirred solution of general intermediate 21 (0.45 mmol) and tert-butyl 4-(4,4.5,5-tetramethyl-1,3,2-dioxaborolan-2-yl)-3,6-dihydro-2H-pyridine-1-carboxylate (0.54 mmol) in 1,4-dioxane (3 mL), is added Na₂CO₃ (0.67 mmol) dissolved in H₂O (1.2 mL) at RT. The reaction mixture is purged with argon for 30 min. To the resulting reaction mixture, PdCl₂(dppf) (0.03 g, 0.04 mmol) is added and the reaction mixture is again purged with argon for 20 min. The reaction mixture is stirred in sealed tube at 100° C. for 16 h. The progress of the reaction is monitored by TLC. After completion of reaction, the reaction mixture is filtered through celite. The filtrate is diluted with cold water and extracted with ethyl acetate. The combined organic layer is washed with water and brine, dried over anhydrous Na₂SO₄ and concentrated under reduced pressure to afford desired product.

### Step-2

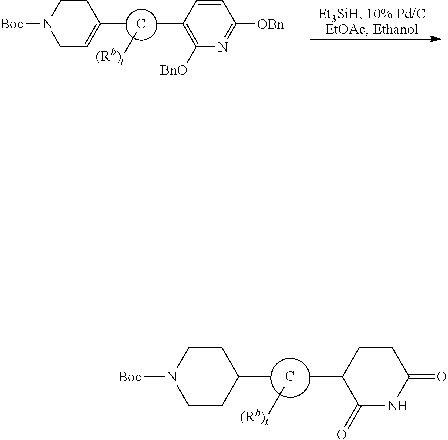

To a stirred solution of intermediate from step 2 (0.25 mmol) in EtOAc (0.5 mL) and EtOH (0.5 mL), is added 10% Pd/C (0.03 g) followed by triethyl silane (0.29 g, 2.55 mmol) at RT. The reaction mixture is stirred at RT for 16 h. The progress of the reaction is monitored by TLC. After completion of reaction, the reaction mixture is filtered through celite. The filtrate is concentrated under reduced pressure to afford desired product

### Step-3

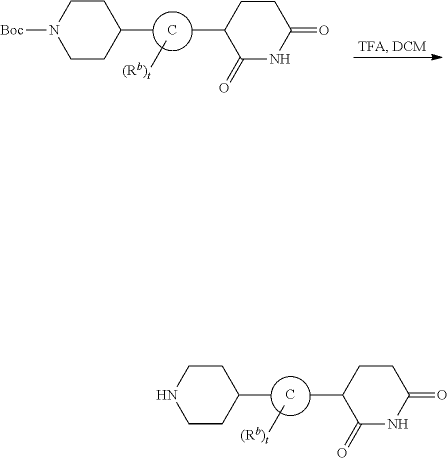

To a stirred solution of intermediate from step 2 (0.16 mmol) in DCM (2 mL) is added 30% TFA in DCM (4 mL) at 0° C. and the reaction mixture is stirred at 0° C. for 5 min. The reaction is allowed to warm to RT and stirred for 4 h. The progress of the reaction is monitored by TLC. After completion of reaction, the reaction mixture is concentrated under reduced pressure to afford desired product.

### Step-4

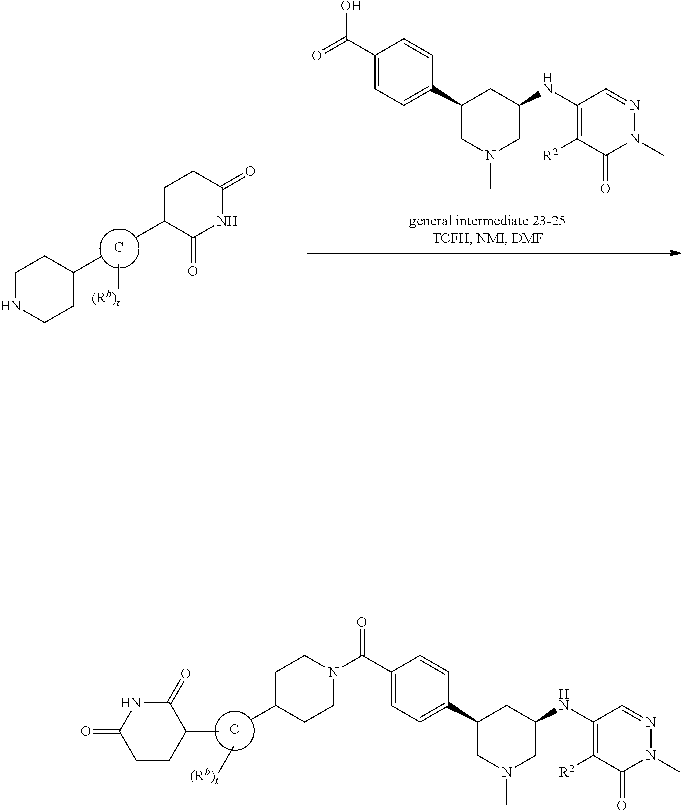

To a stirred solution of intermediate from step 3 (0.22 mmol) in DMF (3 mL), is added general intermediate 23-25 (0.22 mmol) followed by N-methyl imidazole (1.10 mmol) and the reaction mixture is stirred at RT for 10 min. To the resulting reaction mixture, N,N,N′,N′-tetramethylchloroformamidinium hexafluorophosphate (0.09 g, 0.33 mmol) is added and reaction is stirred at 85° C. for 16 h. The progress of the reaction is monitored by TLC. After completion of reaction, the reaction mixture is diluted with ice-cold water. The precipitated solid is filtered to yield crude compound which is purified by prep HPLC to afford desired product.

### General Procedure 6

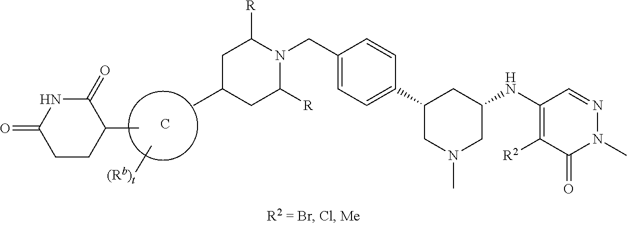

In various embodiments,

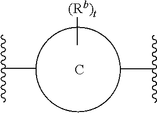

is

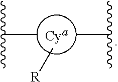

### Step 1

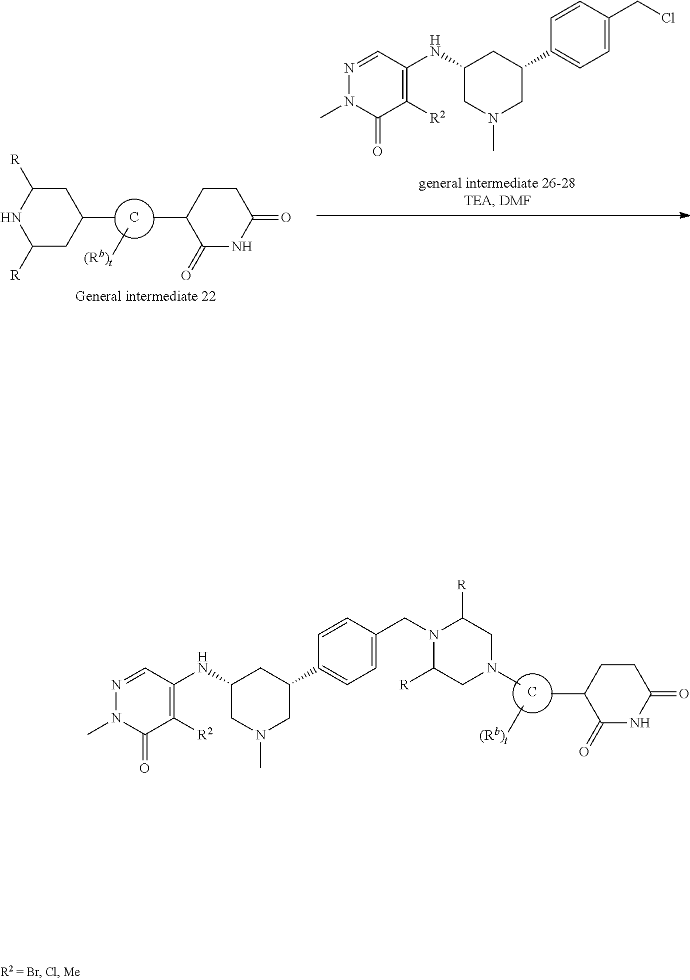

To a stirred solution of of the amine intermediates (0.34 mmol, from general procedure 5) in DMF (1 mL) is added triethylamine (1.00 mmol) at RT. The reaction mixture is stirred at RT for 5 min. To this is added intermediate 26-28 (0.31 mmol) at RT and the reaction mixture is stirred at 100° C. for 16 h. The progress of the reaction is monitored by TLC. After completion of reaction, the reaction mixture is diluted with water and extracted with DCM. The combined organic layer is dried over anhydrous Na₂SO₄ and concentrated under reduced pressure. The crude compound is purified by prep HPLC to afford the desired product.

### General Procedure 7

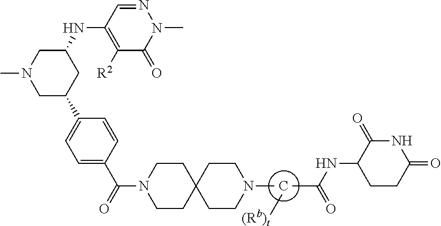

In various embodiments,

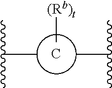

is

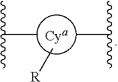

### Step 1

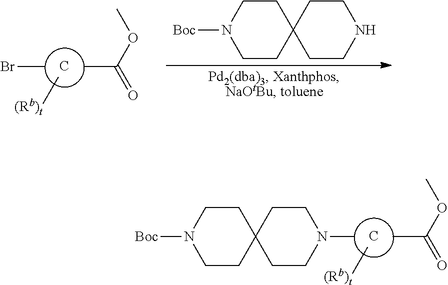

To a stirred solution of substituted bromo-aryl ester (9.25 mmol) and tert-butyl 3,9-diazaspiro[5.5]undecane-3-carboxylate (11.11 mmol) in toluene (20 mL), is added cesium carbonate (27.77 mmol) at RT and the reaction mixture is purged with argon for 30 min. To the resulting reaction mixture, Pd₂(dba)₃ (0.93 mmol) and 4,5-bis(diphenylphosphino)-9,9-dimethylxanthene (1.85 mmol) are added and the reaction mixture is again purged with argon for 20 min. The reaction mixture is stirred in a sealed tube at 100° C. for 16 h. The progress of the reaction is monitored by TLC. After completion of reaction, the reaction mixture is filtered through a celite pad and then concentrated under reduced pressure to obtain crude product. The crude is purified by flash chromatography (silica gel) using 50% EtOAc in heptane as eluent to afford the desired intermediate.

### Step 2

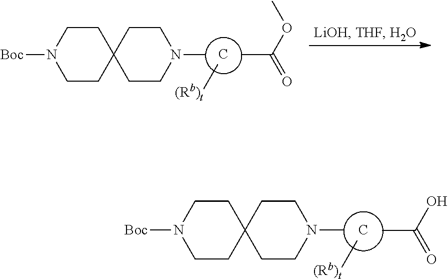

To a stirred solution of intermediate from step 1 (3.00 mmol) in tetrahydrofuran (20 mL), methanol (20 mL) and water (10 mL) is added lithium hydroxide (0.15 g, 6.10 mmol). The reaction mixture is heated to 55° C. for 16 h. The progress of the reaction is monitored by TLC. After completion of reaction, the reaction mixture is concentrated under reduced pressure, the residue is diluted with water, and the aqueous layer is washed with diethyl ether followed by acidification with 2N HCl solution to pH 2. The precipitated solid is filtered and washed with heptane to afford the desired intermediate.

### Step 3

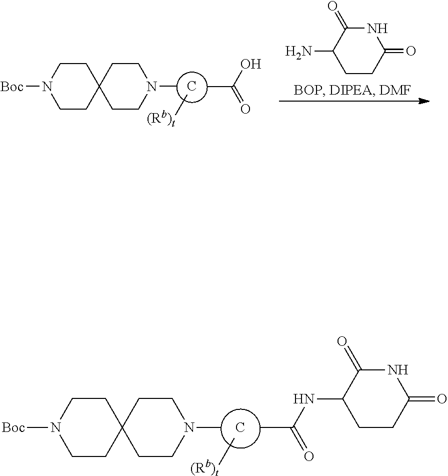

To a stirred solution of intermediate from step 2 (2.66 mmol) in DMF (20 mL) is added N,N-diisopropylethylamine (7.96 mmol) and 3-aminopiperidine-2,6-dione (2.90 mmol), respectively, and the reaction mixture is stirred at RT for 10 min. To this is added BOP (2.24 mmol) and the reaction mixture is stirred at RT for 16 h. The progress of the reaction is monitored by TLC. After completion of reaction, the reaction mixture is diluted with ice-cold water and extracted with DCM. The organic layer is dried over anhydrous sodium sulphate, filtered, and concentrated under reduced pressure to obtain crude. The crude compound is purified by combi flash chromatography (silica) where the product is eluted with 10% MeOH in DCM to afford the desired intermediate.

### Step 4

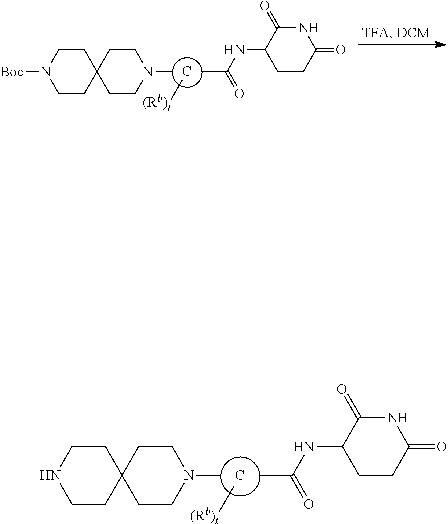

To a stirred solution of intermediate from step 3 (1.46 mmol) in dichloromethane (30 mL) is added TFA (5 mL) at 0° C. and the reaction mixture is stirred at RT for 2 h. The progress of the reaction is monitored by TLC. After completion of the reaction, the reaction mixture is concentrated under reduced pressure and then the residue is triturated with DCM and diethyl ether, respectively, to afford the desired intermediate.

### Step 5

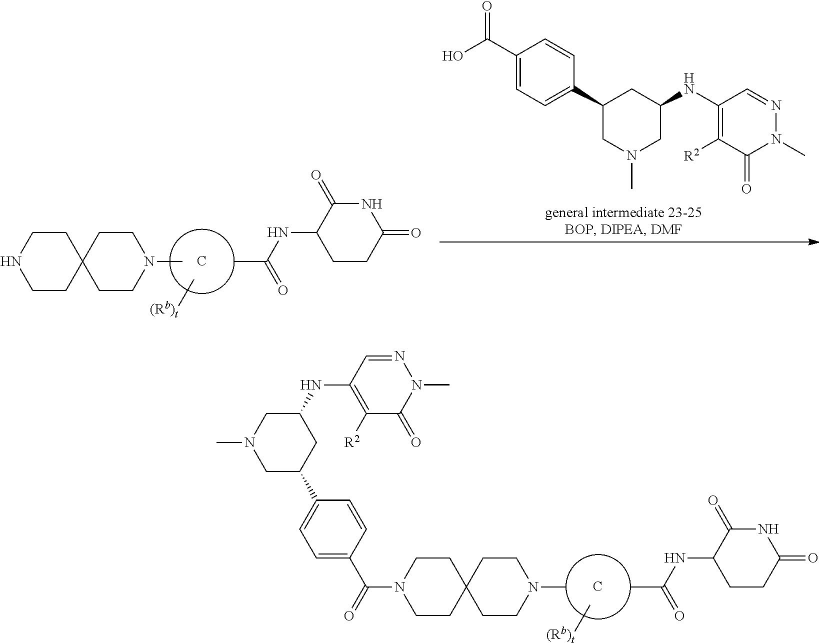

To a stirred solution of intermediate from step 4 (0.35 mmol) in DMF (5 mL) is added N,N-diisopropylethylamine (1.4 mmol) and general intermediate 23-25 (0.14 g, 0.35 mmol). The reaction mixture is stirred at RT for 10 min then BOP (0.35 g, 0.768 mmol) is added and the reaction mixture is stirred at RT for 2 h. The progress of the reaction is monitored by TLC. After completion of reaction, the reaction mixture is diluted with ice-cold water and extracted with ethyl acetate. The organic layer is dried over anhydrous sodium sulphate, filtered, and concentrated under reduced pressure to obtain crude product. The crude compound is purified by flash chromatography (silica) with 8% MeOH in DCM as eluent to afford the desired product.

### General Procedure 8

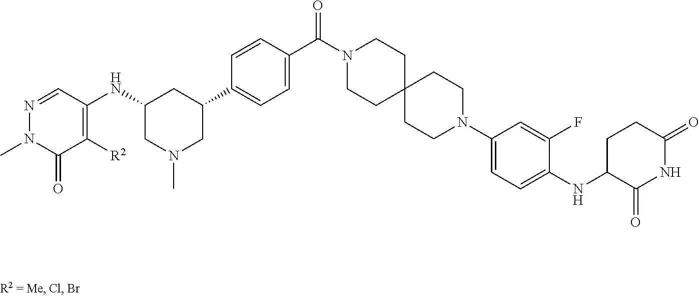

### Step 1

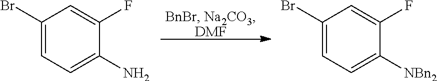

To the stirred solution of 4-bromo-2-flouroamiline (10.31 mmol) in DMF (20 mL), is added sodium carbonate (30.90 mmol) and stirred for 5 min. Then benzyl bromide (21.70 mmol) is added drop wise to the reaction mixture and stirred at 110° C. for 4 h. After the completion of the reaction, the reaction mixture is extracted with EtOAc and washed with cold water. The organic layer is dried over anhydrous sodium sulphate, filtered, and concentrated under reduced pressure to obtain crude product. The crude is purified by flash chromatography (silica) where the product is eluted with 5% EtOAc in heptane to afford N,N-dibenzyl-4-bromo-2-fluoroaniline as an off-white solid.

### Step 2

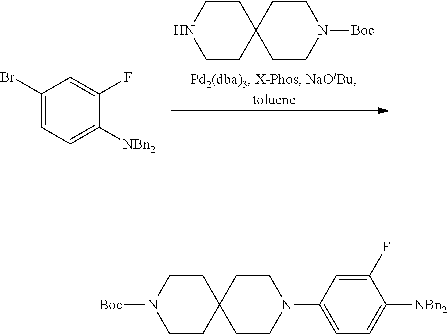

To the stirred solution of N,N-dibenzyl-4-bromo-2-fluoroaniline (5.29 mmol, from step 1) and tort-butyl 3,9-diazaspiro[5.5]undecane-3-carboxylate (5.29 mmol) in toluene (20 mL) is added sodium tert-butoxide (15.88 mmol) and purged with argon gas for 15 min. Then 4,5-bis(diphenylphosphino)-9,9-dimethylxanthene (1.05 mmol) is added followed by Pd₂(dba)₃ (0.52 mmol) to the reaction mixture under argon atmosphere and further purged with argon gas for 15 min. The reaction mixture is stirred at 110° C. for 16 h. The progress of the reaction is monitored by TLC. After completion of the reaction, the reaction mixture is filtered through a celite pad, and the filtrate is concentrated under reduced pressure to obtain crude. The crude is purified by flash chromatography (silica) where the product is eluted with 30% EtOAc in heptane to afford tert-butyl 9-(4-(dibenzylamino)-3-fluorophenyl)-3,9-diazaspiro[5.5]undecane-3-carboxylate as a brown solid.

### Step 3

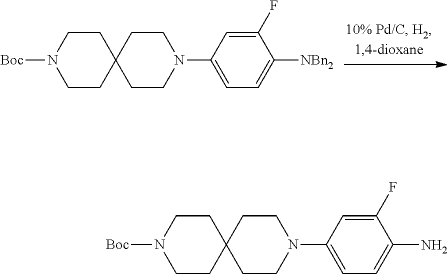

To a stirred solution of tert-butyl 9-(4-(dibenzylamino)-3-fluorophenyl)-3,9-diazaspiro[5.5]undecane-3-carboxylate (2.70 mmol, from step 2) in 1,4-dioxane (5 mL) is added 10% Pd/C (0.50 g) and the reaction mixture is stirred under hydrogen atmosphere (50 psi) at 80° C. for 12 h. The progress of the reaction is monitored by TLC. After completion of the reaction, the reaction mixture is passed through a celite pad, and the filtrate is concentrated under reduced pressure to obtain the crude product. The crude is purified by silica-gel column chromatography where the product is eluted with 70% EtOAc in heptane to afford tert-butyl 9-(4-amino-3-fluorophenyl)-3,9-diazaspiro[5.5]undecane-3-carboxylate as a brown semi solid.

### Step 4

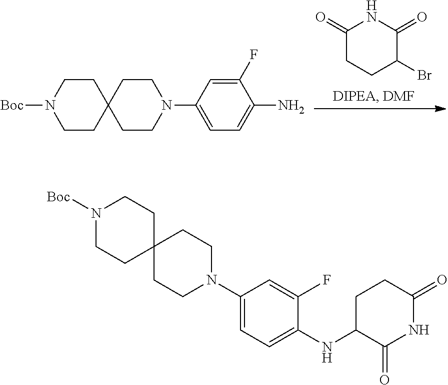 [SMILES: CC(C)(C)OC(=O)N1CCC2(CC1)CCN(c1ccc(N)c(F)c1)CC2 ; O=C1CCC(Br)C(=O)N1 ; CC(C)(C)OC(=O)N1CCC2(CC1)CCN(c1ccc(NC3CCC(=O)NC3=O)c(F)c1)CC2]

To the stirred solution of tert-butyl 9-(4-amino-3-fluorophenyl)-3,9-diazaspiro[5.5]undecane-3-carboxylate (1.87 mmol, from step 3) in DMF (10 mL), is added N,N-diisopropylethylamine (9.33 mmol) and stirred for 2 min. Then 3-bromopiperidine-2,6-dione (2.80 mmol) is added to the reaction mixture and the reaction mixture is stirred at 100° C. for 12 h. The progress of the reaction is monitored by TLC. After completion of the reaction, the reaction mixture is filtered, and the filtrate is concentrated to obtain crude product. The crude is purified by silica-gel column chromatography where the product is eluted with 60% EtOAc in heptane to afford tert-butyl 9-(4-((2,6-dioxopiperidin-3-yl)amino)-3-fluorophenyl)-3,9-diazaspiro[5.5]undecane-3-carboxylate as a brown solid.

### Step 5

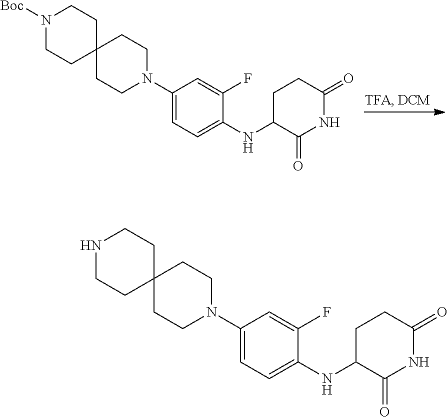 [SMILES: CC(C)(C)OC(=O)N1CCC2(CC1)CCN(c1ccc(NC3CCC(=O)NC3=O)c(F)c1)CC2 ; O=C1CCC(Nc2ccc(N3CCC4(CCNCC4)CC3)cc2F)C(=O)N1]

To a stirred solution of tert-butyl 9-(4-((2,6-dioxopiperidin-3-yl)amino)-3-fluorophenyl)-3,9-diazaspiro[5.5]undecane-3-carboxylate (0.35 g, 0.73 mmol, from step 4) in DCM (20 mL) is added TFA (1.5 mL) at 0° C. and the reaction mixture is stirred at RT for 2 h. The progress of the reaction is monitored by TLC. After completion of the reaction, the reaction mixture is concentrated under reduced pressure, the residue obtained is triturated with ether and heptane, respectively, to afford 3-((2-fluoro-4-(3,9-diazaspiro[5.5]undecan-3-yl)phenyl)amino)piperidine-2,6-dione TFA as an off-white solid.

### Step 6

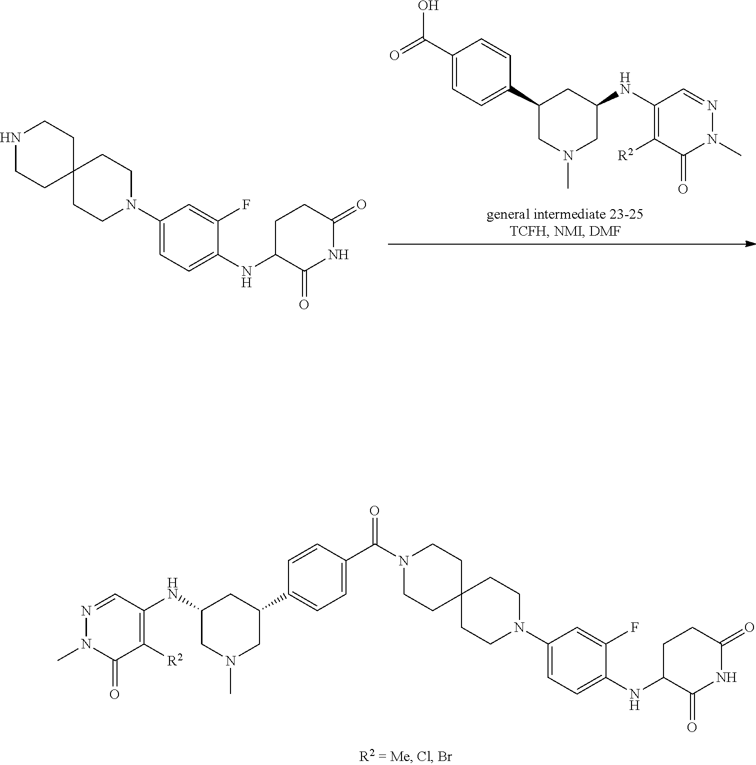

To a stirred solution of 3-((2-fluoro-4-(3,9-diazaspiro[5.5]undecan-3-yl)phenyl)amino)-piperidine-2,6-dione (0.44 mmol, from step 5) and general intermediate 23-25 (0.44 mmol) in DMF (5 mL) are added 1-methylimidazole (2.33 mmol) and TCFH (0.93 mmol), respectively. The reaction mixture is stirred at RT for 16 h. The progress of the reaction is monitored by TLC. After completion of reaction, the reaction mixture is diluted with ice-cold water and extracted with ethyl acetate. The combined organic layer is dried over anhydrous sodium sulphate, filtered, and concentrated under reduced pressure to obtain crude. The crude is purified by silica-gel column chromatography with 10% MeOH in DCM as eluent to afford desired product.

### General Procedure 9

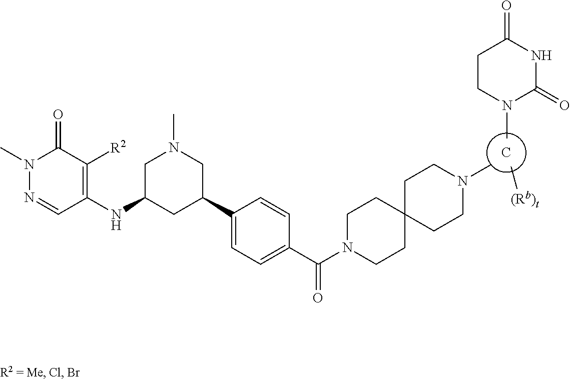

In various embodiments,

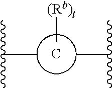

is

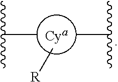

### Step 1

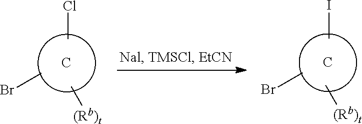

To a stirred solution of bromo chloro quinoline (16.49 mmol) in propionitrile (40 mL) are added sodium iodide (32.50 mmol) and chlorotrimethylsilane (2.3 mL), respectively, at 0° C. The reaction mixture is stirred at 100° C. for 16 h. The progress of the reaction is monitored by TLC. After completion the reaction, the reaction mixture is basified using aq. sodium hydroxide solution and then extracted with diethyl ether. The combined organic layer is dried over anhydrous sodium sulphate, filtered, concentrated under reduced pressure to obtain crude compound. The crude is purified by silica gel column chromatography (30% EtOAc in heptane) to obtain the desired intermediates.

### Step 2

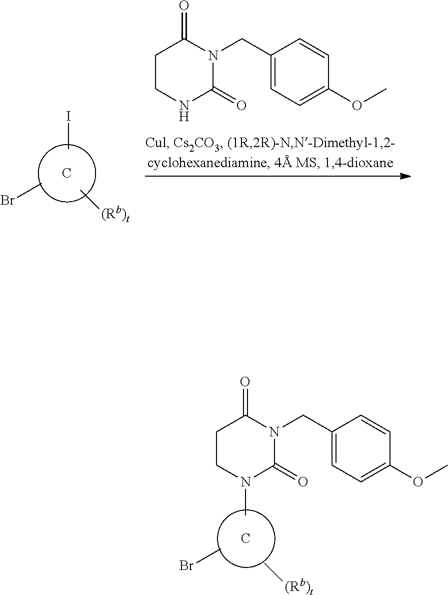

To a stirred solution of intermediate from step 1 (5.98 mmol) and cesium carbonate (4.88 g, 14.90 mmol) in 1,4-dioxane (20 ml) were added MS 4 Å (200 mg) and 3-(4-methoxybenzyl)dihydropyrimidine-2,4 (1H,3H)-dione (6.60 mmol, from step 6), and the reaction mixture is purged with argon for 20 min. To the resulting reaction mixture, copper (I) iodide (1.17 mmol) and (1R,2R)—N,N′-dimethyl-1,2-cyclohexanediamine (1.20 mmol) is added, and the reaction mixture is again purged with argon for 10 min. The reaction mixture is stirred in a sealed tube at 80° C. for 16 h. The progress of the reaction is monitored by TLC. After completion of reaction, the reaction mixture is filtered through a celite pad. The filtrate is diluted with cold water and extracted with ethyl acetate. The combined organic layer is washed with water and brine, dried over anhydrous Na₂SO₄ filtered, and concentrated under reduced pressure to obtain crude. The crude compound is purified by silica gel column chromatography to afford the desired intermediates.

### Step 3

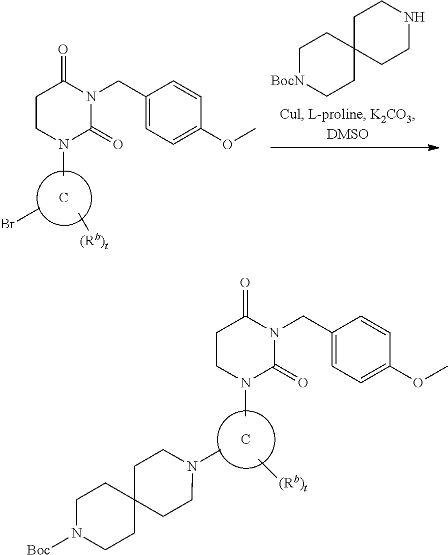

To a stirred solution of intermediate from step 2 (4.12 mmol) and tert-butyl 3,9-diazaspiro[5.5]undecane-3-carboxylate (4.12 mmol) in DMSO (18 mL), is added potassium carbonate (12.40 mmol) at RT and the reaction mixture is purged with argon for 30 min. To the resulting reaction mixture, L-Proline (0.81 mmol) is added and purged with argon for 10 min and then copper (I) iodide (0.40 mmol) is added and the reaction mixture is again purged with argon for 5 min. The reaction mixture is stirred at 110° C. for 16 h. The progress of the reaction is monitored by TLC. After completion of reaction, the reaction mixture is filtered through a celite pad. The filtrate is diluted with cold water and extracted with ethyl acetate. Combined organic layer is washed with water and brine, dried over anhydrous Na₂SO₄, filtered, and concentrated under reduced pressure to obtain crude product. The crude compound is purified by silica gel column chromatography to afford the desired intermediate.

### Step 4

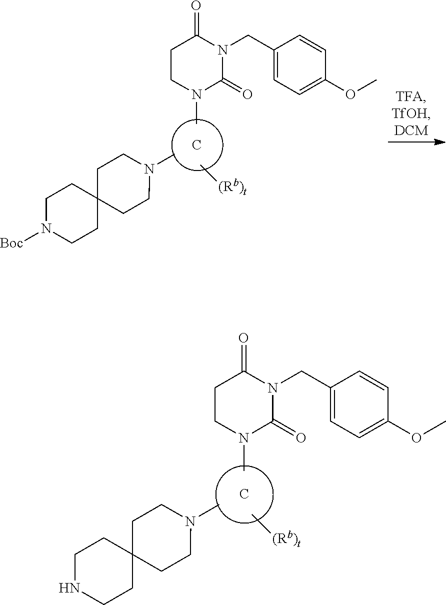

To a stirred cooled solution of intermediate from step 3 (0.65 mmol) in dichloromethane (8 mL), is added trifluoromethanesulfonic acid (4 mL) and TFA (4 mL) at 0° C. The reaction mixture is then stirred at RT for 3 h. The progress of the reaction is monitored by TLC. After completion of reaction, the reaction mixture is concentrated under reduced pressure to afford the desired intermediate.

### Step 5

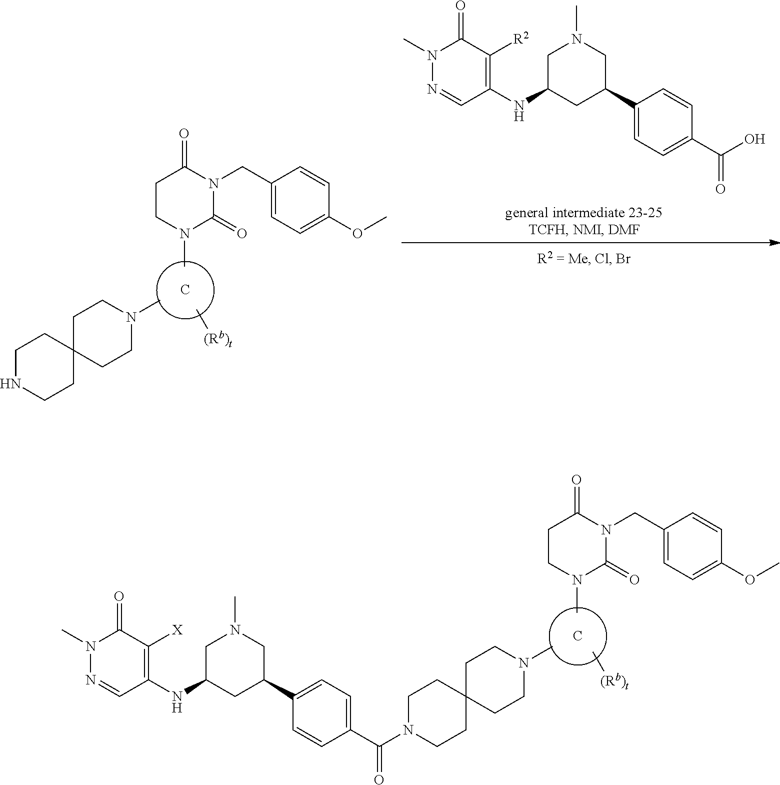

To a stirred solution of intermediate from step 4 (0.30 mmol) in DMF (5 mL), is added 1-methylimidazole (1.00 mmol) and general intermediate 23-25 (0.25 mmol), respectively, and the reaction mixture is stirred at RT for 10 min. To this is added TCFH (0.37 mmol) and the reaction mixture is stirred at 90° C. The progress of the reaction is monitored by TLC. After completion of reaction, ice cold water is added to the reaction mixture and then filtered and dried to obtain the crude compound. The crude is purified by preparative HPLC to afford desired product.

### Step 6: 3-(4-methoxybenzyl)dihydropyrimidine-2,4(1H,3H)-dione

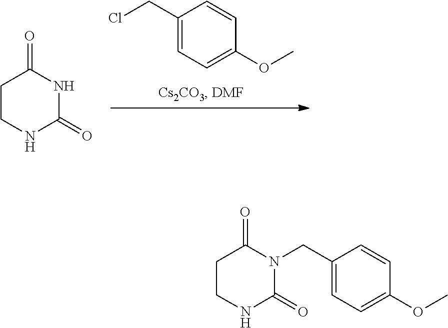

To a stirred solution of hexahydropyrimidine-2,4-dione (43.82 mmol) in DMF (60 mL) is added cesium carbonate (64.40 mmol) and 4-methoxybenzyl chloride (65.7 mmol) the reaction mixture is stirred at 30° C. for 12 h. The progress of the reaction is monitored by TLC. After completion of reaction, the reaction mixture is quenched with ice-cold water. The precipitated solid is filtered, washed with water and heptane, respectively and dried under reduced pressure to afford 3-(4-methoxybenzyl)dihydropyrimidine-2,4(1H,3H)-dione as a white solid. LC-MS: m/z 234.80 [M+H]⁺.

### General Procedure 10

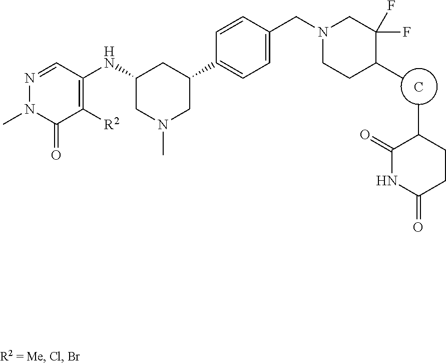

### Step 1: tert-butyl 3,3-difluoro-4-(((trifluoromethyl)sulfonyl)oxy)-3,6-dihydropyridine-1(2H)-carboxylate

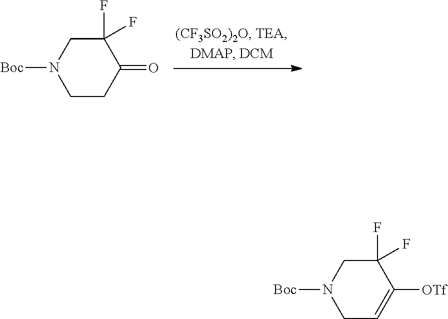 [SMILES: CC(C)(C)OC(=O)N1CCC(=O)C(F)(F)C1 ; CC(C)(C)OC(=O)N1CC=C(OS(=O)(=O)C(F)(F)F)C(F)(F)C1]

To a stirred solution of tert-butyl 3,3-difluoro-4-oxopiperidine-1-carboxylate (4.25 mmol) in dichloromethane (10 mL) is added triethylamine (13 mmol) and 4-dimethylaminopyridine (0.42 mmol) at 0° C. Trifluoromethanesulfonic anhydride (6.35 mmol) is added to the reaction mixture and reaction mixture is stirred at RT for 16 h. The progress of the reaction is monitored by TLC. After completion of the reaction, the reaction mixture is quenched with saturated aq. sodium bicarbonate solution, and then extracted with DCM. The organic layer is dried over anhydrous sodium sulphate, filtered, and concentrated under reduced pressure to obtain crude product. The crude is purified by silica-gel column chromatography where the product is eluted with 30% EtOAc in heptane to afford tert-butyl 3,3-difluoro-4-(trifluoromethylsulfonyloxy)-2,6-dihydropyridine-1-carboxylate as a yellow liquid. ¹H NMR (400 MHz, CD₃OD) δ=6.57 (br s, 1H), 4.29-4.24 (m, 2H), 4.02 (t, J=11.2 Hz, 2H), 1.49 (s, 9H).

### Step 2

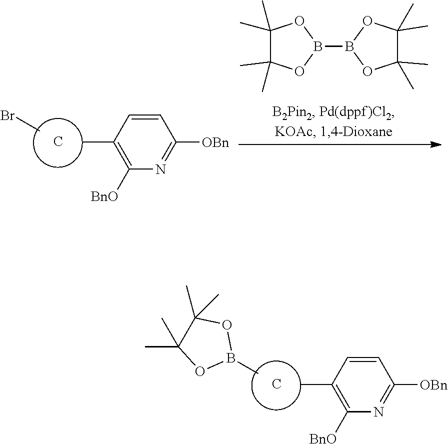

To a stirred solution of general intermediate 1 (24.00 mmol) in 1,4-dioxane (35 mL) is added potassium acetate (23.00 mmol) and the reaction mixture is purged with argon for 10 min. To the resulting reaction mixture, [1,1 bis(diphenylphosphino)ferrocene]dichloropalladium (II) (0.78 mmol) is added and the reaction mixture is again purged with argon for 10 min. The reaction mixture is stirred at 100° C. for 16 h in a sealed tube. The progress of the reaction is monitored by TLC. After completion of reaction, the reaction mixture is filtered through a celite pad, and the filtrate is concentrated under reduced pressure to obtain crude compound. The crude is purified by flash chromatography (silica) where the product is eluted with 10% ethyl acetate in heptane to afford the desired intermediate.

### Step 3

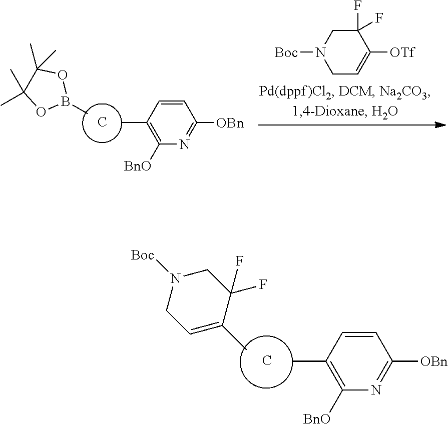

To a stirred solution of intermediate form step 2 (1.08 mmol) in 1,4-dioxane (10 mL) and water (2 mL), is added 2,6-dibenzyloxy-3-[3-(4,4,5,5-tetramethyl-1,3,2-dioxaborolan-2-yl)phenyl]-pyridine (1.29 mmol, from step 1) and sodium carbonate (2.72 mmol) and the reaction mixture is purged under nitrogen atmosphere for 10 min. 1,1′-bis(diphenylphosphino)ferrocene]dichloropalladium (II), complex with dichloromethane (0.10 mmol) is added to the reaction mixture under nitrogen atmosphere. The reaction mixture is stirred at 100° C. for 16 h. The progress of the reaction is monitored by TLC. After completion of the reaction, the reaction mixture is cooled to room temperature, diluted with water, and extracted with EtOAc. The organic layer is washed with brine solution, dried over anhydrous sodium sulphate, filtered, and concentrated under reduced pressure to obtain crude product. The crude is purified by flash chromatography (silica) where the product is eluted with 10% ethyl acetate in heptane to afford the desired intermediate.

### Step 4

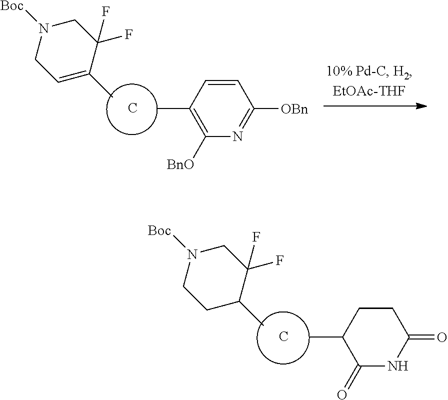

To a stirred solution of intermediate from step 3 (0.42 mmol) in ethyl acetate (2 mL) and tetrahydrofuran (2 mL) is added 10% Pd/C (0.2 g) at RT. The reaction mixture is stirred under hydrogen pressure (100 psi) at RT for 16 h. The progress of the reaction is monitored by TLC. The reaction mixture is filtered through a celite pad washing with 10% MeOH in DCM. The combined filtrate is concentrated under reduced pressure and the obtained crude compound is purified by flash chromatography (silica) where the product is eluted with 2% MeOH in DCM to afford the desired intermediate.

### Step 5

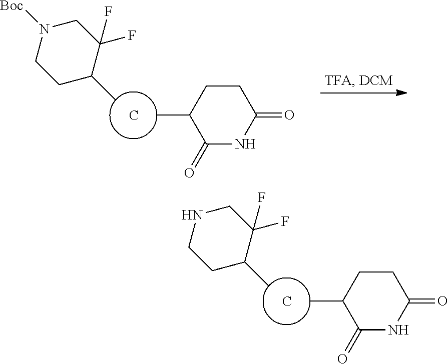

To a stirred solution intermediate from step 4 (0.36 mmol) in dichloromethane (2 mL), is added TFA (0.5 mL) at 0° C. and the reaction mixture is stirred at RT for 2 h. The progress of the reaction is monitored by TLC. After the completion of the reaction, the reaction mixture is concentrated under reduced pressure and triturated with DCM and diethyl ether, respectively, to afford the desired intermediate as TFA salt.

### Step 6

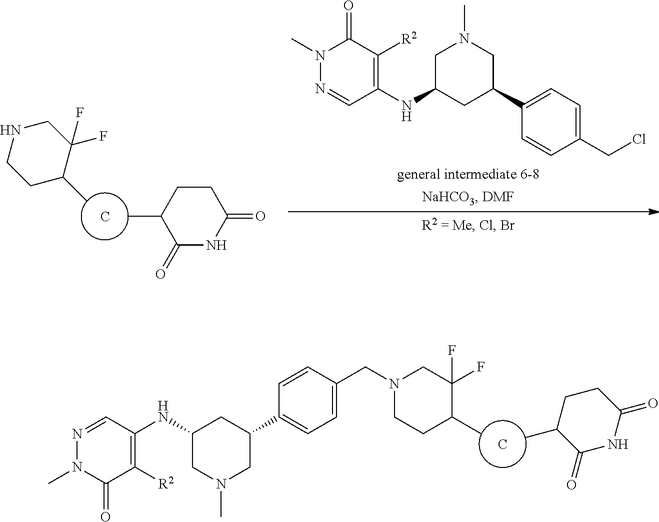

To a stirred solution of intermediate from step 5 (0.22 mmol) in N,N-dimethyl formamide (2 mL), is added sodium bicarbonate (1.11 mmol) followed by general intermediate 26-28 (0.22 mmol). Reaction is stirred at 85° C. for 16 h. The progress of the reaction is monitored by TLC and LCMS. After completion of the reaction, ice cold water is added to the reaction mixture and the precipitated solids were filtered, dried, and purified by preparative HPLC to afford the desired product.

### General Procedure 11

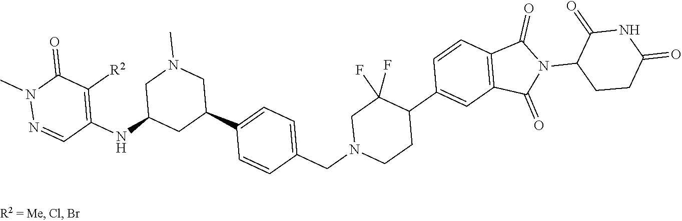

### Step 1: 5-bromo-2-(2,6-dioxopiperidin-3-yl)isoindoline-1,3-dione

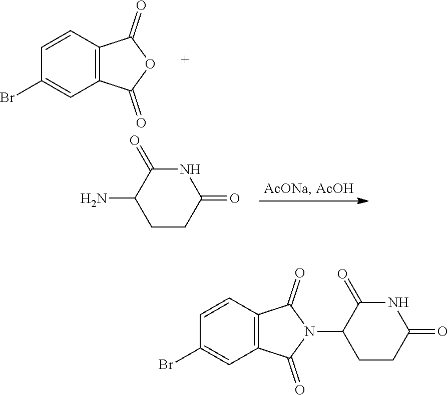

To a stirred solution of 5-bromoisobenzofuran-1,3-dione (8.80 mmol) and 3-aminopiperidine-2,6-dione (11.40 mmol) in acetic acid (30 mL) is added sodium acetate (8.71 mmol) and the reaction mixture is stirred at 120° C. for 16 h. The progress of the reaction is monitored by TLC. After completion of the reaction, the reaction mixture is concentrated under reduced pressure to get crude compound, which is diluted with 100 mL of water and stirred at RT for 3 h. The reaction mixture is filtered under reduced pressure and dried under vacuum to afford 5-bromo-2-(2,6-dioxopiperidin-3-yl) isoindoline-1,3-dione as an off white solid. LCMS: m/z 336.80 [M+H]⁺.

### Step 2: 2-(2,6-dioxopiperidin-3-yl)-5-(4,4,5,5-tetramethyl-1,3,2-dioxaborolan-2-yl)isoindoline-1,3-dione

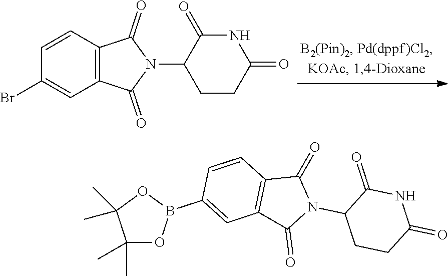 [SMILES: O=C1CCC(N2C(=O)c3ccc(Br)cc3C2=O)C(=O)N1 ; CC1(C)OB(c2ccc3c(c2)C(=O)N(C2CCC(=O)NC2=O)C3=O)OC1(C)C]

A stirred solution of 5-bromo-2-(2,6-dioxopiperidin-3-yl) isoindoline-1,3-dione (2.96 mmol, from Step 1), bis(pinacolato)diboron (8.90 mmol) and potassium acetate (8.88 mmol) in 1,4-dioxane (20 mL) is purged with argon for 15 min. To the resulting mixture, [1,1′-bis(diphenylphosphino)ferrocene]dichloropalladium (II) (0.29 mmol) is added and the reaction mixture is again purged with argon for 15 min. The reaction mixture is stirred at 100° C. for 16 h in a sealed tube. The progress of the reaction is monitored by TLC. After completion of reaction, the reaction mixture is filtered through a celite pad, and the filtrate is concentrated under reduced pressure to afford 2-(2,6-dioxopiperidin-3-yl)-5-(4,4,5,5-tetramethyl-1,3,2-dioxaborolan-2-yl) isoindoline-1,3-dione as a dark brown solid. LCMS: m/z 385.22 [M+H]⁺.

### Step 3: tert-butyl 4-(2-(2,6-dioxopiperidin-3-yl)-1,3-dioxoisoindolin-5-yl)-3,3-difluoro-3,6-dihydropyridine-1(2H)-carboxylate

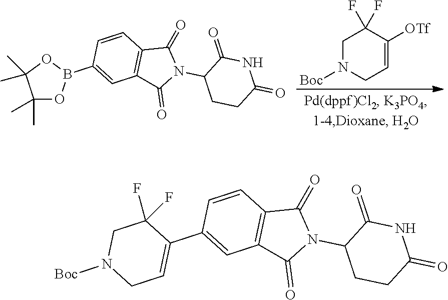 [SMILES: CC1(C)OB(c2ccc3c(c2)C(=O)N(C2CCC(=O)NC2=O)C3=O)OC1(C)C ; CC(C)(C)OC(=O)N1CC=C(OS(=O)(=O)C(F)(F)F)C(F)(F)C1 ; CC(C)(C)OC(=O)N1CC=C(c2ccc3c(c2)C(=O)N(C2CCC(=O)NC2=O)C3=O)C(F)(F)C1]

A stirred solution of tert-butyl 3,3-difluoro-4-(((trifluoromethyl)sulfonyl)oxy)-3,6-dihydropyridine-1(2H)-carboxylate (1.36 mmol, from step of 1 general procedure 10), 2-(2,6-dioxopiperidin-3-yl)-5-(4,4,5,5-tetramethyl-1,3,2-dioxaborolan-2-yl) isoindoline-1,3-dione (1.36 mmol, from step 2) and potassium carbonate (4.08 mmol) in 1,4-dioxane (5 ml) and water (0.3 mL) is purged with argon for 15 min. To the resulting reaction mixture, PdCl₂(dtbpf) (0.13 mmol) is added, and the reaction mixture is again purged with argon for 15 min. The reaction mixture is stirred at 100° C. for 16 h in sealed tube. The progress of the reaction is monitored by TLC. After completion of reaction, the reaction mixture is filtered through a celite pad. The filtrate is concentrated under reduced pressure to obtain crude, which is purified by silica-gel column chromatography using 40% EtOAc in heptane as eluent to afford tert-butyl 4-(2-(2,6-dioxopiperidin-3-yl)-1,3-dioxoisoindolin-5-yl)-3,3-difluoro-3,6-dihydropyridine-1(2H)-carboxylate as a yellow liquid. LCMS: m/z 474.00 [M−H]⁻.

### Step 4: tert-butyl 4-(2-(2,6-dioxopiperidin-3-yl)-1,3-dioxoisoindolin-5-yl)-3,3-difluoropiperidine-1-carboxylate

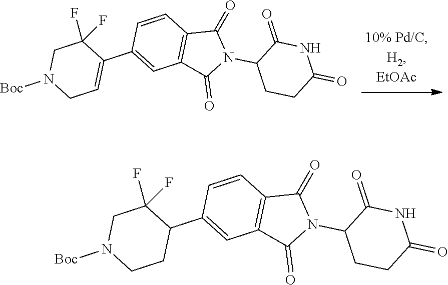 [SMILES: CC(C)(C)OC(=O)N1CC=C(c2ccc3c(c2)C(=O)N(C2CCC(=O)NC2=O)C3=O)C(F)(F)C1 ; CC(C)(C)OC(=O)N1CCC(c2ccc3c(c2)C(=O)N(C2CCC(=O)NC2=O)C3=O)C(F)(F)C1]

To a stirred solution of tert-butyl 4-(2-(2,6-dioxopiperidin-3-yl)-1,3-dioxoisoindolin-5-yl)-3,3-difluoro-3,6-dihydropyridine-1 (2H)-carboxylate (0.35 mmol, from step 3) in ethyl acetate (5 mL) is added 10% Pd/C (0.15 g) at RT. The reaction mixture is stirred at RT under H₂ atmosphere (100 psi) for 16 h. The progress of the reaction is monitored by TLC and LCMS. After completion of reaction, the reaction mixture is filtered through a celite pad, and the filtrate is concentrated under reduced pressure to obtain crude. The crude compound is purified by silica-gel column chromatography using 5% MeOH in DCM as eluent to afford tert-butyl 4-(2-(2,6-dioxopiperidin-3-yl)-1,3-dioxoisoindolin-5-yl)-3,3-difluoropiperidine-1-carboxylate as a yellow liquid. LCMS: m/z 476.28 [M−H]⁻.

### Step 5: 5-(3,3-difluoropiperidin-4-yl)-2-(2,6-dioxopiperidin-3-yl)isoindoline-1,3-dione

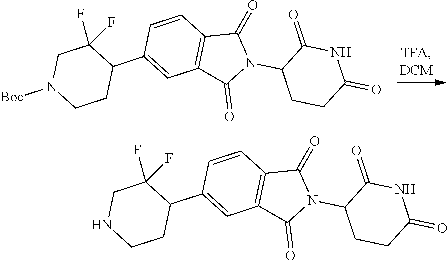

To a cooled stirring solution of tert-butyl 4-(2-(2,6-dioxopiperidin-3-yl)-1,3-dioxoisoindolin-5-yl)-3,3-difluoropiperidine-1-carboxylate (0.20 mmol, from step 4) in dichloromethane (2 mL), is added TFA (0.1 mL) at 0° C. The reaction mixture is warmed to RT and stirred for 3 h. The progress of the reaction is monitored by TLC. After completion of reaction, the reaction mixture is concentrated under reduced pressure to afford crude compound, which is triturated with diethyl ether and heptane, respectively, to afford 5-(3,3-difluoropiperidin-4-yl)-2-(2,6-dioxopiperidin-3-yl) isoindoline-1,3-dione TFA as a colourless liquid, which is used as such for the next step reaction. LCMS: m/z 378.27 [M+H]⁻.

### Step 6

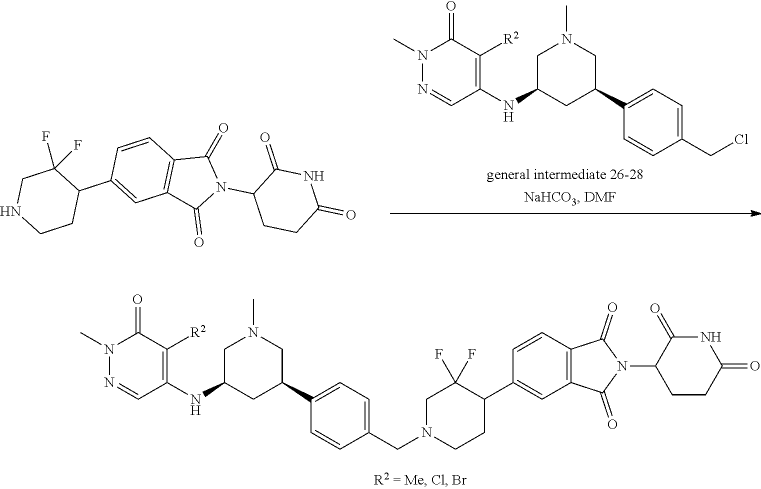

To a stirred solution of 5-(3,3-difluoropiperidin-4-yl)-2-(2,6-dioxopiperidin-3-yl) isoindoline-1,3-dione (0.22 mmol, from step-5) in N,N-dimethyl formamide (2 mL), is added sodium bicarbonate (1.11 mmol) followed by general intermediate 26-28 (0.22 mmol). The reaction is stirred at 85° C. for 16 h. The progress of the reaction is monitored by TLC and LCMS. After completion of the reaction, ice cold water is added to the reaction mixture and the precipitated solids were filtered, dried, and purified by preparative HPLC to afford the desired product.

### General Procedure 12

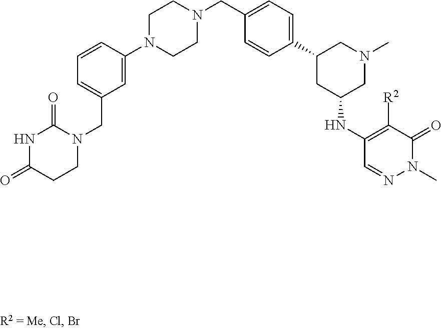

### Step 1: tert-butyl 4-(3-cyanophenyl)piperazine-1-carboxylate

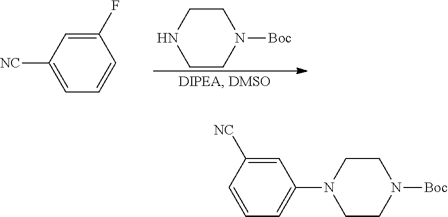 [SMILES: N#Cc1cccc(F)c1 ; CC(C)(C)OC(=O)N1CCNCC1 ; CC(C)(C)OC(=O)N1CCN(c2cccc(C#N)c2)CC1]

To a stirred solution of 3-fluorobenzonitrile (12 mmol) in DMSO (15 mL) is added tert-butyl 3,9-diazaspiro[5.5]undecane-3-carboxylate (9.80 mmol) followed by N,N-diisopropylethylamine (25.00 mmol) and the reaction mixture is stirred at 100° C. for 16 h. The progress of the reaction is monitored by TLC. After completion of reaction, the reaction mixture is diluted with ice cold water to afford tert-butyl 4-(3-cyanophenyl)piperazine-1-carboxylate which is used as such for the next reaction.

### Step 2: tert-butyl 4-(3-(aminomethyl)phenyl)piperazine-1-carboxylate

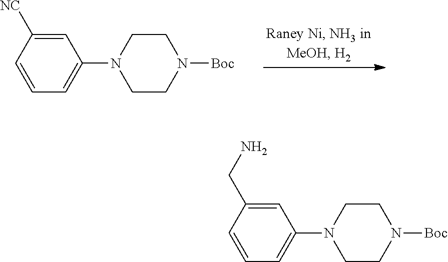 [SMILES: CC(C)(C)OC(=O)N1CCN(c2cccc(C#N)c2)CC1 ; CC(C)(C)OC(=O)N1CCN(c2cccc(CN)c2)CC1]

To a stirred solution of tert-butyl tert-butyl 4-(3-cyanophenyl)piperazine-1-carboxylate (2.00 g, 5.62 mmol, from step 1) in methanol (15 mL) is added Raney nickel (34.07 mmol) followed by 7M ammonia in MeOH (5 mL) and the reaction mixture is stirred at 60 psi H₂ gas pressure at RT for 16 h. The progress of the reaction is monitored by TLC. After completion of reaction, the reaction mixture is filtered through celite. The filtrate is concentrated under reduced pressure to afford tert-butyl 4-(3-(aminomethyl)phenyl)piperazine-1-carboxylate which is used such as for the next reaction.

### Step 3: tert-butyl 4-(3-(((2-cyanoethyl)amino)methyl)phenyl)piperazine-1-carboxylate

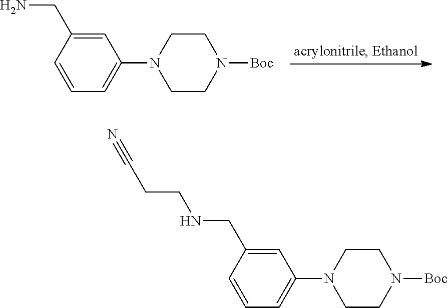

To a stirred solution of tert-butyl 4-(3-(aminomethyl)phenyl)piperazine-1-carboxylate (3.10 mmol, from step 2) in ethanol (11 mL) is added acrylonitrile (2.43 mmol) at RT. The reaction mixture is stirred at 90° C. for 3 h. The progress of the reaction is monitored by TLC. After completion of reaction, the reaction mixture is cooled at RT. The white solid formed is filtered using Buchner funnel to afford tert-butyl 4-(3-(((2-cyanoethyl)amino)methyl)phenyl)piperazine-1-carboxylate which is used as such for the next reaction.

### Step 4: tert-butyl 4-(3-((N-(2-cyanoethyl)cyanamido)methyl)phenyl)piperazine-1-carboxylate

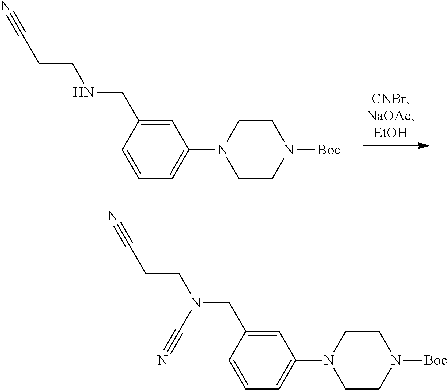

To a stirred solution of tert-butyl 4-(3-(((2-cyanoethyl)amino)methyl)phenyl)piperazine-1-carboxylate (0.60 mmol, from step 3) in ethanol (3 mL) is added sodium acetate (0.60 mmol) followed by cyanogen bromide (0.60 mmol) at RT and the reaction mixture is stirred at RT for 16 h. The progress of the reaction is monitored by TLC. After completion of reaction, the reaction mixture is cooled to RT, diluted with water and extracted with ethyl acetate. The combined organic layer is washed with water and brine, dried over anhydrous Na₂SO₄ and concentrated under reduced pressure to afford tert-butyl 4-(3-((N-(2-cyanoethyl)cyanamido)methyl)phenyl)piperazine-1-carboxylate which is used as such for the next reaction.

### Step 5: 1-(3-(piperazin-1-yl)benzyl)dihydropyrimidine-2,4(1H,3H)-dione

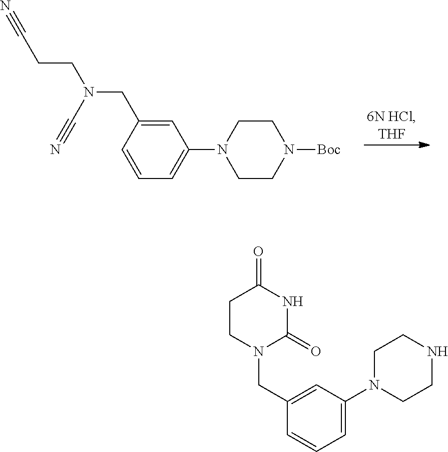 [SMILES: CC(C)(C)OC(=O)N1CCN(c2cccc(CN(C#N)CCC#N)c2)CC1 ; O=C1CCN(Cc2cccc(N3CCNCC3)c2)C(=O)N1]

To a stirred solution of tert-butyl 4-(3-((N-(2-cyanoethyl)cyanamido)methyl)phenyl)piperazine-1-carboxylate (0.22 mmol, from step 4) in THF (2 mL) is added 6N HCl (1 mL) and the reaction mixture is stirred at 100° C. for 2 h. The progress of the reaction is monitored by TLC. After completion of reaction, the reaction mixture is concentrated under reduced pressure to afford 1-(3-(piperazin-1-yl)benzyl)dihydropyrimidine-2,4(1H,3H)-dione which is used as such for the next reaction.

### Step 6

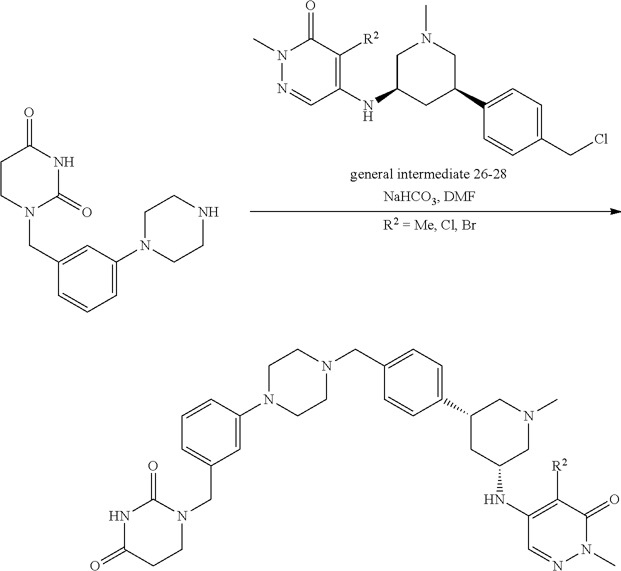

To a stirred solution of 1-(3-(piperazin-1-yl)benzyl)dihydropyrimidine-2,4(1H,3H)-dione (0.22 mmol, from step 5) in N,N-dimethyl formamide (2 mL), is added sodium bicarbonate (1.11 mmol) followed by general intermediate 26-28 (0.22 mmol). The reaction is stirred at 85° C. for 16 h. The progress of the reaction is monitored by TLC and LCMS. After completion of the reaction, ice cold water is added to the reaction mixture and the precipitated solids are filtered, dried, and purified by preparative HPLC to afford the desired product.

### General Procedure 13

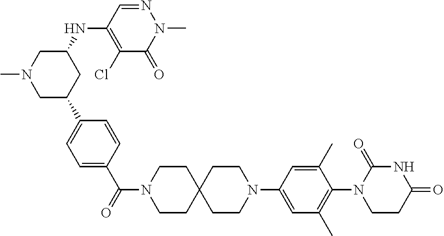 [SMILES: Cc1cc(N2CCC3(CCN(C(=O)c4ccc([C@H]5C[C@@H](Nc6cnn(C)c(=O)c6Cl)CN(C)C5)cc4)CC3)CC2)cc(C)c1N1CCC(=O)NC1=O]

### Step 1: 1-(4-bromo-2,6-dimethylphenyl)dihydropyrimidine-2,4(1H,3H)-dione

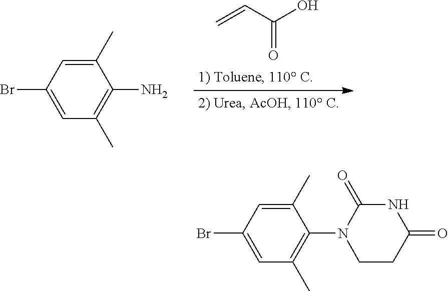

To the stirred solution of 4-bromo-2,6-dimethylaniline (24.99 mmol) in toluene (40 mL), is added acrylic acid (117 mmol) and stirred for 110° C. for 16 h. The progress of the reaction is monitored by TLC. After completion of the reaction, the reaction mixture is concentrated under reduced pressure to obtain crude which is dissolved in acetic acid (30 mL). To this mixture urea (6.3 g, 100 mmol) is added and reaction heated to 110° C. for 16 b. The progress of the reaction is monitored by TLC. The reaction mixture is concentrated under reduced pressure, basified with aq. sodium bicarbonate, the precipitated solids were filtered and dried under vacuum to afford 1-(4-bromo-2,6-dimethylphenyl)dihydropyrimidine-2,4(1H,3H)-dione as a yellow solid.

### Step 2

Desired product is obtained by using 1-(4-bromo-2,6-dimethylphenyl)dihydropyrimidine-2,4(1H,3H)-dione (from step 2) and following steps 3 to 5 in general procedure 9.

### General Procedure 14

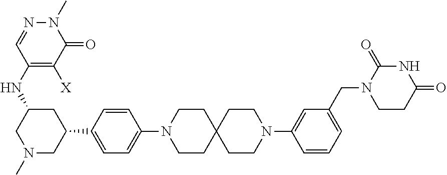

### Step-1: 1-(3-bromobenzyl)-3-(4-methoxybenzyl)dihydropyrimidine-2,4(1H,3H)-dione

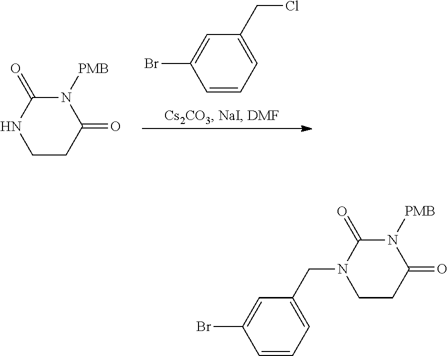

To a stirred solution of 1-bromo-3-(chloromethyl)benzene (13 mmol) in DMF (20 mL) is added cesium carbonate (26 mmol) followed by 3-[(4-methoxyphenyl)methyl]hexahydropyrimidine-2,4-dione (7.9 mmol) and sodium iodide (6.60 mmol) at RT. The reaction mixture is stirred at 110° C. for 16 b. The progress of the reaction is monitored by TLC & LCMS. The reaction mixture is cooled to RT, water is added and extracted with ethyl acetate (3×20 mL). The combined organic layer is dried over anhydrous sodium sulphate, filtered, and concentrated under reduced pressure to obtain crude product, which is purified by flash chromatography (silica) with 70% ethyl acetate in heptane as an eluent to afford 1-[(3-bromophenyl)methyl]-3-[(4-methoxyphenyl)methyl]hexahydropyrimidine-2,4-dione as a colourless liquid. LCMS: m/z 403.0, 405.0 [M+H]⁺.

### Step 2: tert-butyl 9-(3-((3-(4-methoxybenzyl)-2,4-dioxotetrahydropyrimidin-1 (2H)-yl)methyl)phenyl)-3,9-diazaspiro[5.5]undecane-3-carboxylat

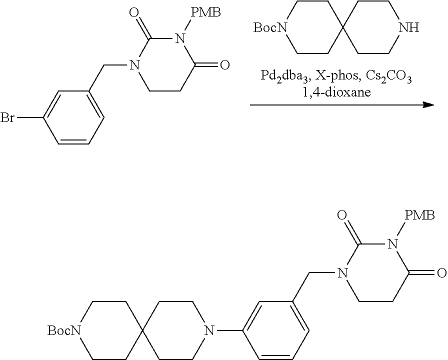

To a stirred solution of 1-[(3-bromophenyl)methyl]-3-[(4-methoxyphenyl)methyl]hexa-hydropyrimidine-2,4-dione (2.1 mmol, from step 1) in 1,4-dioxane (20 mL), caesium carbonate (11 mmol) is added at RT. The reaction mixture is purged with argon for 30 min. To the resulting reaction mixture, 2-dicyclohexylphosphino-2′,6′-dimethoxybiphenyl (0.37 mmol) and tris(dibenzylideneacetone) dipalladium (0) (0.18 mmol) are added and reaction again purged with argon for 20 min. The reaction mixture is stirred in a sealed tube at 110° C. for 16 h. The progress of the reaction is monitored by TLC and LCMS. After completion of reaction, the reaction mixture is filtered through a pad of celite and washed with ethyl acetate (100 mL). The filtrate is diluted with cold water and extracted with ethyl acetate. The combined organic layer is washed with water and brine, dried over anhydrous sodium sulphate, filtered, and concentrated under reduced pressure to obtain crude. The crude is purified by flash chromatography (silica) using 70% ethyl acetate in heptane as an eluent to afford tert-butyl 9-(3-((3-(4-methoxybenzyl)-2,4-dioxotetrahydropyrimidin-1 (2H)-yl) methyl)phenyl)-3,9-diazaspiro[5.5]undecane-3-carboxylate as a pale yellow gummy solid. LCMS: m/z 577.30 [M+H]⁺.

### Step 3: 1-(3-(3,9-diazaspiro[5.5]undecan-3-yl)benzyl)-3-(4-methoxybenzyl)dihydropyri-midine-2,4(1H,3H)-dione

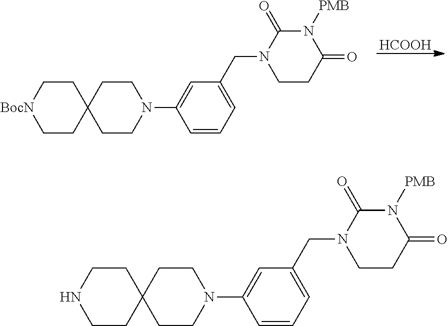

A mixture of tert-butyl 9-(3-((3-(4-methoxybenzyl)-2,4-dioxotetrahydropyrimidin-1 (2H)-yl)methyl)phenyl)-3,9-diazaspiro[5.5]undecane-3-carboxylate (1 mmol, from step 2) in formic acid (10 mL) is stirred at 0° C. for 5 min followed by at RT for 3 h. The progress of the reaction is monitored by TLC. After completion of reaction, the reaction mixture is concentrated under reduced pressure. The obtained crude is triturated with diethyl ether to afford 1-(3-(3,9-diazaspiro[5.5]undecan-3-yl)benzyl)-3-(4-methoxybenzyl)dihydropyri-midine-2,4(1H,3H)-dione as a brown colour gum. LCMS: m/z 477.30 [M+H]⁺.

### Step 4: tert-butyl ((3R,5R)-5-(4-(9-(3-((3-(4-methoxybenzyl)-2,4-dioxotetrahydropyri-midin-1(2H)-yl)methyl)phenyl)-3,9-diazaspiro[5.5]undecan-3-yl)phenyl)-1-methylpiper-idin-3-yl)carbamate

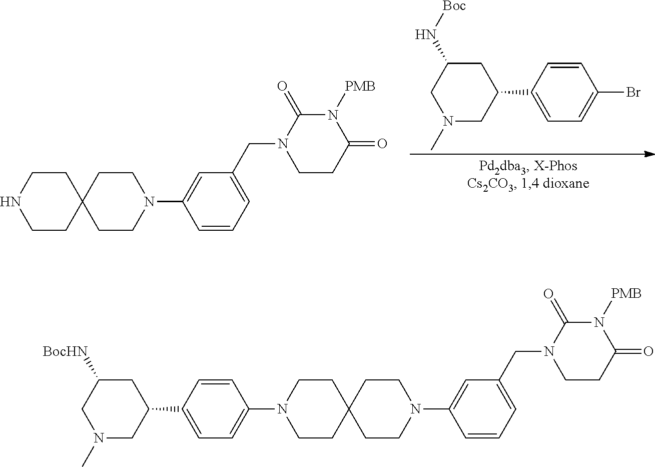

To a stirred solution of tert-butyl ((3R,5R)-5-(4-bromophenyl)-1-methylpiperidin-3-yl)carbamate (1.76 mmol) and 1-(3-(3,9-diazaspiro[5.5]undecan-3-yl)benzyl)-3-(4-methoxybenzyl)dihydropyrimidine-2,4(1H,3H)-dione (1.23 mmol, from step 3) in 1,4-dioxane (6 mL) is added cesium carbonate (5.28 mmol) at RT and the reaction mixture is purged with argon for 20 min. To the resulting reaction mixture, tris(dibenzylideneacetone) dipalladium (0) (0.17 mmol) and 2-dicyclohexylphosphino-2′,4′,6′-triisopropylbiphenyl (0.17 g, 0.35 mmol) is added and the reaction mixture is again purged with argon for 10 min. The reaction mixture is stirred in a sealed tube at 100° C. for 16 h. The progress of the reaction is monitored by TLC and LCMS. After completion of the reaction mixture, the reaction mixture is filtered through a pad of celite. The filtrate is concentrated under reduced pressure and the obtained crude is purified by silica-gel column chromatography using 10% MeOH in DCM as eluent to afford of tert-butyl ((3R,5R)-5-(4-(9-(3-((3-(4-methoxybenzyl)-2,4-dioxotetrahydropyrimidin-1(2H)-yl)methyl)phenyl)-3,9-diazaspiro[5.5]undecan-3-yl)phenyl)-1-methylpiperidin-3-yl)carbamate as a light yellow solid. LC-MS: m/z 765.30 [M+H]⁺.

### Step 5: 1-(3-(9-(4-((3R,5R)-5-amino-1-methylpiperidin-3-yl)phenyl)-3,9-diazaspiro-[5.5]undecan-3-yl)benzyl)-3-(4-methoxybenzyl)dihydropyrimidine-2,4(1H,3H)-dione

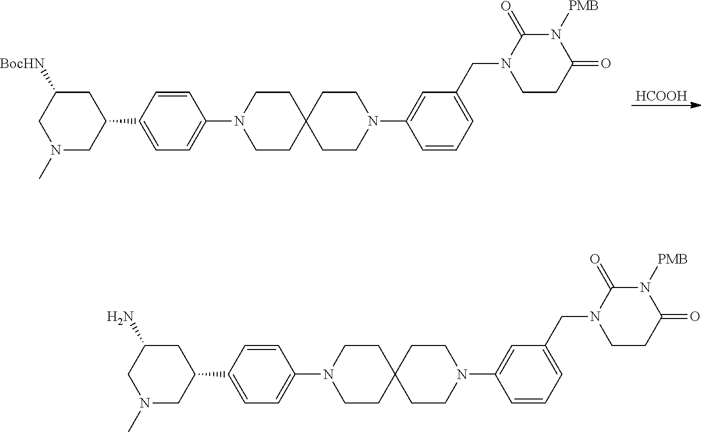

A solution of tert-butyl ((3R,5R)-5-(4-(9-(3-((3-(4-methoxybenzyl)-2,4-dioxotetrahydropyrimidin-1(2H)-yl)methyl)phenyl)-3,9-diazaspiro[5.5]undecan-3-yl)phenyl)-1-methylpiperidin-3-yl)carbamate (0.28 mmol, from step 4) in formic acid (3 mL) is stirred at RT for 3 h. The progress of the reaction is monitored by TLC and LCMS. After completion the reaction mixture, the reaction mixture is concentrated under reduced pressure to obtain crude. The crude is quenched with saturated aq. NaHCO₃ and extracted with DCM. The organic layer is dried over anhydrous sodium sulphate, filtered, and concentrated under reduced pressure to afford 1-(3-(9-(4-((3R,5R)-5-amino-1-methylpiperidin-3-yl)phenyl)-3,9-diazaspiro[5.5]undecan-3-yl)benzyl)-3-(4-methoxybenzyl)dihydropyrimidine-2,4(1H,3H)-dione as a light yellow solid. LC-MS: m/z 665.50 [M+H]⁺.

### Step 6

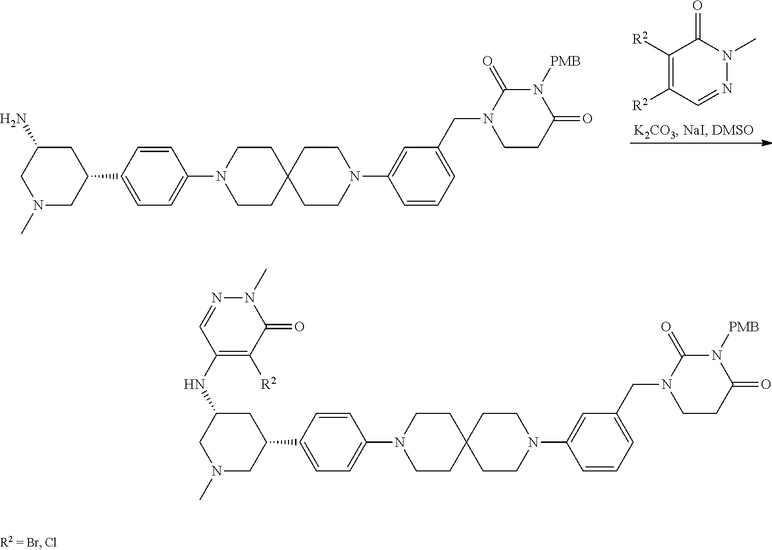

To a stirred solution of 1-(3-(9-(4-((3R,5R)-5-amino-1-methylpiperidin-3-yl)phenyl)-3,9-diazaspiro[5.5]undecan-3-yl)benzyl)-3-(4-methoxybenzyl)dihydropyrimidine-2,4(1H,3H)-dione (0.10 mmol, from step 5) in DMSO (3 mL) is added 4,5-dibromo-2-methylpyridazin-3 (2H)-one (0.12 mmol), potassium carbonate (0.21 mmol) and sodium iodide (0.05 mmol), respectively, at RT. The reaction mixture is stirred at 110° C. for 2 h in a microwave. The progress of the reaction is monitored by TLC and LCMS. After completion of the reaction, the reaction mixture is filtered through a pad of celite. The filtrate is concentrated under reduced pressure to obtain crude, which is purified by silica-gel column chromatography using 15% MeOH in DCM as eluent to afford the desired intermediate.

### Step 7

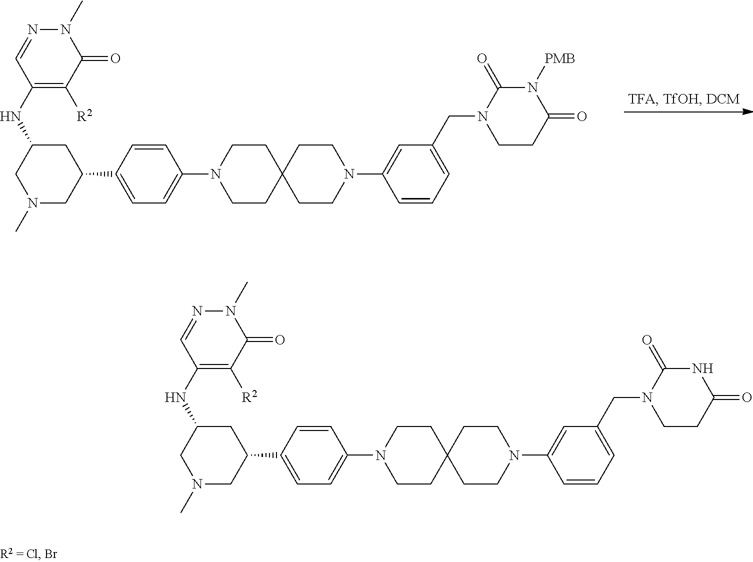

To a stirred solution of intermediate from step 6 (0.04 mmol) in DCM (0.5 mL) is added trifluoroacetic acid (0.2 mL) and triflic acid (0.05 mL) at 0° C. The reaction mixture is stirred at RT for 2 h. The progress of the reaction is monitored by TLC. After completion of the reaction, the reaction mixture is concentrated under reduced pressure to obtain crude, which is triturated with diethyl ether and purified by preparative HPLC to afford the desired product.

### General Procedure 15 (Unless Noted Otherwise, LINKER is Linker)

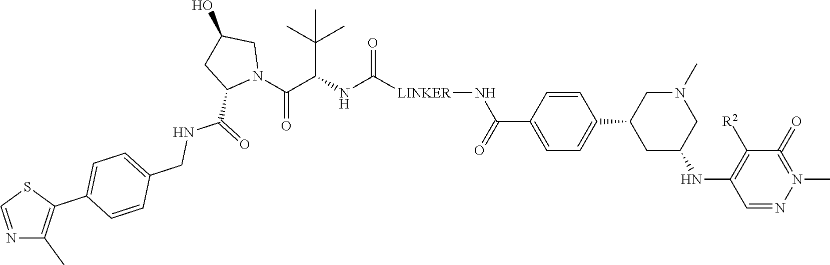

### Step 1

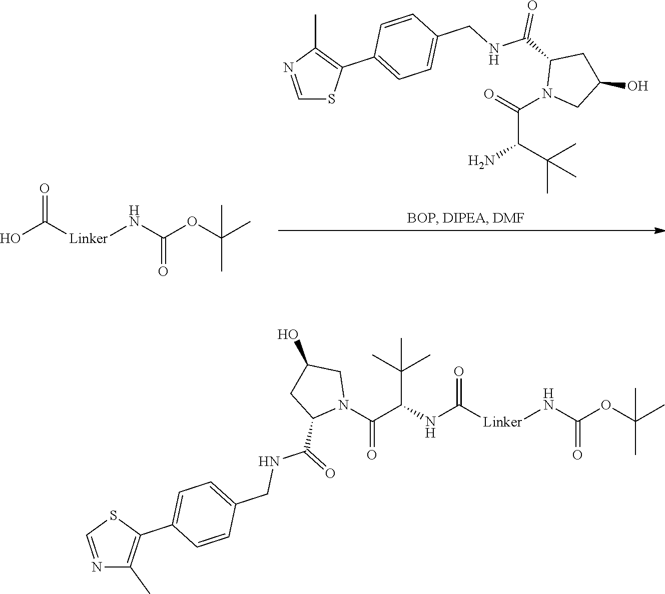

To a stirred solution of the N-Boc amino carboxylic acid linker (0.34 mmol) in DMF (5 mL) is added N,N-diisopropylethylamine (2 mmol) and stirred at RT for 5 min. To the reaction mixture, (2S,4R)-1-((S)-2-amino-3,3-dimethylbutanoyl)-4-hydroxy-N-(4-(4-methylthiazol-5-yl)benzyl)pyrrolidine-2-carboxamide (0.34 mmol) is added at 0° C. and then stirred at RT for 10 min. Then BOP (0.25 g, 0.54 mmol) is added to the reaction mixture and stirred at RT for 2 h. Progress of the reaction is monitored by TLC and LCMS. After completion of the reaction, the reaction mixture is extracted with ethyl acetate. The organic layer is dried over anhydrous sodium sulphate, filtered, and concentrated under reduced pressure to get crude product. The crude is purified by flash chromatography (silica gel) where the product is eluted with 10% MeOH in DCM to afford the desired intermediate.

### Step 2

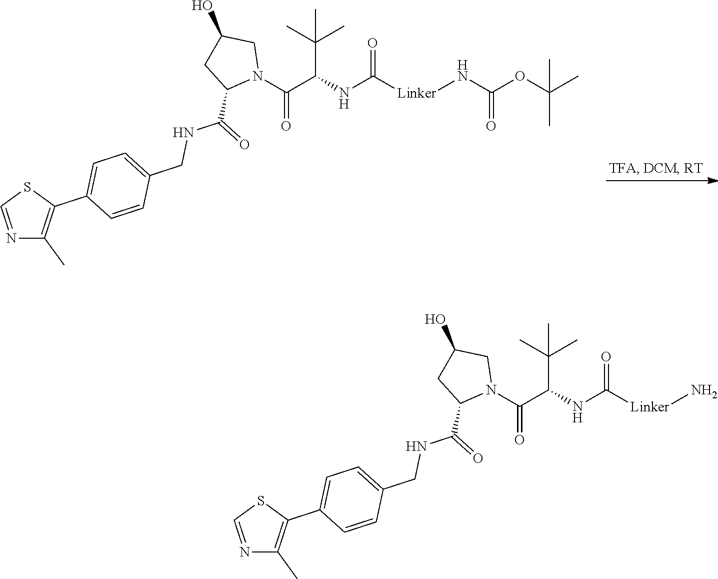

To a stirred solution of intermediate from step 1 (0.17 mmol) in dichloromethane (5 mL) is added TFA (0.2 mL) and reaction mixture is stirred at 0° C. for 5 min and at RT for 2 h. The progress of the reaction is monitored by TLC and LCMS. After completion of the reaction, the reaction mixture is concentrated under reduced pressure and then triturated with heptane and diethyl ether, respectively, to afford the desired intermediate as a TFA salt.

### Step 3

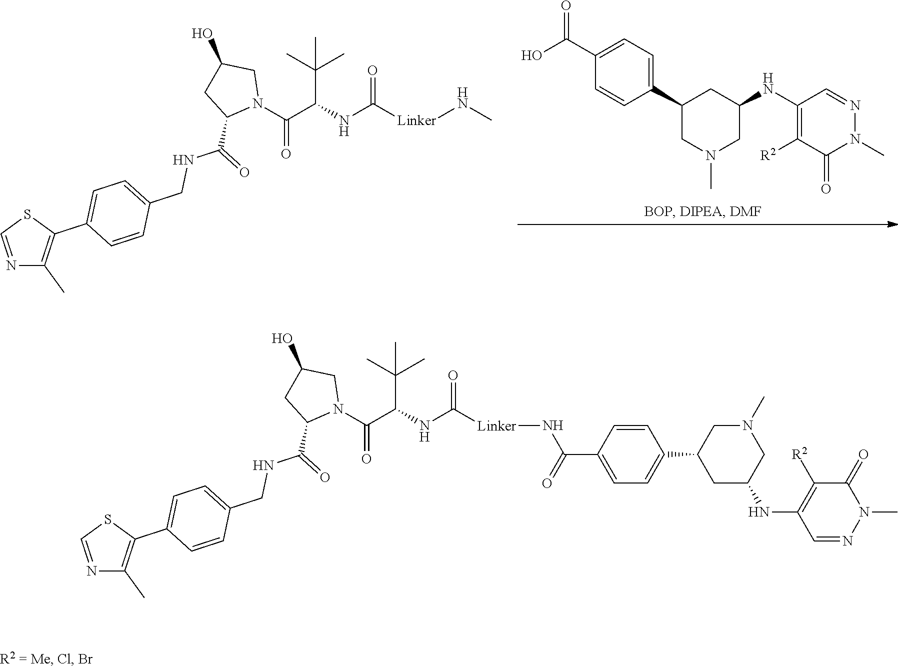

To a stirred solution of general intermediate 23-25 (0.21 mmol) in DMF (5 mL) is added N,N-diisopropylethylamine 0.5 mmol) at RT and stirred at RT for 5 min. To the reaction mixture, intermediate from step 2 (0.17 mmol) and BOP (0.12 g, 0.26 mmol) is added. The reaction mixture is stirred at RT for 2 h. Progress of the reaction is monitored by TLC and LCMS. After completion the reaction mixture is diluted with water and extracted with ethyl acetate. The organic layer is dried over anhydrous sodium sulphate, filtered, and concentrated under reduced pressure to obtain crude product. The crude is purified by preparative HPLC to afford the desired product.

### Preparation of (2S,4R)-1-((S)-2-acetamido-3,3-dimethylbutanoyl)-N-(2-(4-(4-((3R,5R)-5-((5-bromo-1-methyl-6-oxo-1,6-dihydropyridazin-4-yl)amino)-1-methylpiperidin-3-yl)benzamido)-butoxy)-4-(4-methylthiazol-5-yl)benzyl)-4-hydroxypyrrolidine-2-carboxamide (Compound 406)

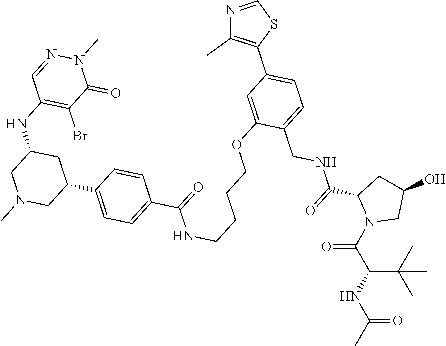 [SMILES: CC(=O)N[C@H](C(=O)N1C[C@H](O)C[C@H]1C(=O)NCc1ccc(-c2scnc2C)cc1OCCCCNC(=O)c1ccc([C@H]2C[C@@H](Nc3cnn(C)c(=O)c3Br)CN(C)C2)cc1)C(C)(C)C]

Compound was synthesized analogously to example 12 using tert-butyl (4-hydroxybutyl)carbamate in step 6. ¹H NMR (400 MHz, DMSO-d₆) δ=8.97 (s, 1H), 8.43 (t, J=5.6 Hz, 2H), 7.94 (d, J=9.6 Hz, 1H), 7.88 (s, 1H), 7.79 (d, J=8.4 Hz, 2H), 7.46 (d, J=8.0 Hz, 1H), 7.33 (d, J=8.4 Hz, 2H), 6.99 (d, J=1.2 Hz, 1H), 6.90 (dd, J=8.0, 1.6 Hz, 1H), 5.81 (d, J=9.2 Hz, 1H), 5.11 (d, J=3.6 Hz, 1H), 4.56-4.44 (m, 2H), 4.37-4.30 (m, 2H), 4.19-4.06 (m, 3H), 3.99-3.91 (m, 1H), 3.66-3.54 (m, 5H), 3.37-3.33 (m, 2H), 3.04-2.93 (m, 2H), 2.84 (dd, J=11.2, 2.7 Hz, 1H), 2.44 (s, 3H), 2.25 (s, 3H), 2.07-1.88 (m, 8H), 1.83-1.68 (m, 5H), 0.91 (s, 9H). LCMS: m/z 962.1 [M+H]⁺.

### Example 12. (2S,4R)-1-((S)-2-acetamido-3,3-dimethylbutanoyl)-N-(2-((6-(4-((3R,5R)-5-((5-bromo-1-methyl-6-oxo-1,6-dihydropyridazin-4-yl)amino)-1-methylpiperidin-3-yl)benzamido)-hexyl)oxy)-4-(4-methylthiazol-5-yl)benzyl)-4-hydroxypyrrolidine-2-carboxamide (Compound 407)

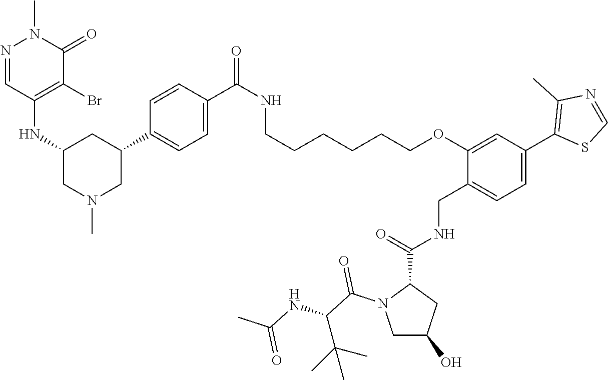 [SMILES: CC(=O)N[C@H](C(=O)N1C[C@H](O)C[C@H]1C(=O)NCc1ccc(-c2scnc2C)cc1OCCCCCCNC(=O)c1ccc([C@H]2C[C@@H](Nc3cnn(C)c(=O)c3Br)CN(C)C2)cc1)C(C)(C)C]

### Step 1: 2-hydroxy-4-(4-methylthiazol-5-yl)benzonitrile

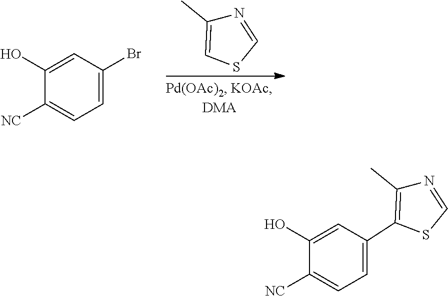

To a stirred solution of 4-bromo-2-hydroxybenzonitrile (10 g, 50.50 mmol) and 4-methylthiazole (10 g, 100.90 mmol) in N,N-dimethylacetamide (150 mL) was added potassium acetate (10.12 g, 101.10 mmol) dissolved in water (10 mL) and the reaction mixture was purged with argon for 30 min. To the resulting reaction mixture, palladium(II) acetate (0.58 g, 2.55 mmol) was added and the reaction mixture was again purged with argon for 20 min. The reaction mixture was stirred at 100° C. for 16 h in a sealed tube. The progress of the reaction was monitored by TLC and LCMS. After completion of the reaction, the reaction mixture was quenched with ice-cold water and the aqueous layer was extracted with ethyl acetate. The organic layer was dried over anhydrous sodium sulphate, filtered, and concentrated under reduced pressure to afford crude product. The crude was purified by silica gel column chromatography using 65% ethyl acetate in hexane as eluent to afford 2-hydroxy-4-(4-methylthiazol-5-yl)benzonitrile (4.5 g, 41%) as a yellow solid. LCMS: m/z 216.90 [M+H]⁺.

### Step 2: 2-(aminomethyl)-5-(4-methylthiazol-5-yl)phenol

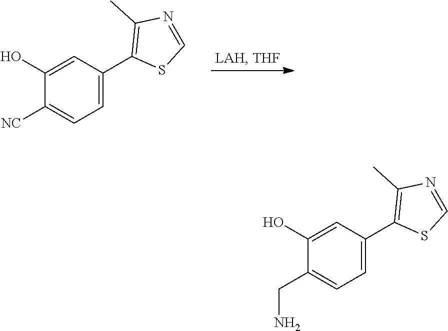

To a stirred solution of 2-hydroxy-4-(4-methylthiazol-5-yl)benzonitrile (3.0 g, 13.87 mmol, from step-1) in tetrahydrofuran (40 mL) was added LAH (1 M in THF, 4 mL, 4 mmol) at 0° C. and the reaction mixture was stirred at RT for 3 h. The progress of the reaction was monitored by TLC. After completion of the reaction, the reaction mixture was quenched with saturated aq. sodium sulphate solution and extracted with ethyl acetate. The organic layer was dried over anhydrous sodium sulphate, filtered, and concentrated under reduced pressure to afford 2-(aminomethyl)-5-(4-methylthiazol-5-yl) phenol (0.60 g, 9%). LCMS: m/z 220.90 [M+H]⁺.

### Step 3: tert-butyl ((S)-1-((2S,4R)-4-hydroxy-2-((2-hydroxy-4-(4-methylthiazol-5-yl)benzyl)carbamoyl)pyrrolidin-1-yl)-3,3-dimethyl-1-oxobutan-2-yl)carbamate

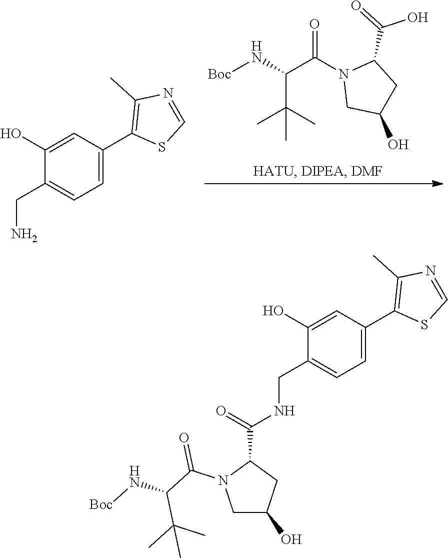 [SMILES: Cc1ncsc1-c1ccc(CN)c(O)c1 ; CC(C)(C)OC(=O)N[C@H](C(=O)N1C[C@H](O)C[C@H]1C(=O)O)C(C)(C)C ; Cc1ncsc1-c1ccc(CNC(=O)[C@@H]2C[C@@H](O)CN2C(=O)[C@@H](NC(=O)OC(C)(C)C)C(C)(C)C)c(O)c1]

To a stirred solution of 2-(aminomethyl)-5-(4-methylthiazol-5-yl) phenol (0.60 g, 2.72 mmol, from step 2) in DMF (6 mL) were added (2S,4R)-1-((S)-2-((tert-butoxycarbonyl)amino)-3,3-dimethylbutanoyl)-4-hydroxypyrrolidine-2-carboxylic acid (0.93 g, 2.72 mmol) and N,N-diisopropylethylamine (2.36 mL), respectively, and the reaction mixture was stirred at RT for 10 min. To this was added BOP (1.52 g, 3.26 mmol) and the reaction mixture was stirred at RT for 4 h. The progress of the reaction was monitored by TLC and LCMS. The reaction mixture was diluted with ice-cold water and extracted with ethyl acetate. The organic layer was dried over anhydrous sodium sulphate, filtered, and concentrated under reduced pressure to obtain crude product. The crude was purified by silica gel column chromatography using 10% ethyl acetate in heptane as eluent to afford tert-butyl ((S)-1-((2S,4R)-4-hydroxy-2-((2-hydroxy-4-(4-methylthiazol-5-yl)benzyl)carbamoyl)pyrrolidin-1-yl)-3,3-dimethyl-1-oxobutan-2-yl)carbamate (0.60 g, 19%). LCMS: m/z 547.00 [M+H]⁺.

### Step 4: (2S,4R)-1-((S)-2-amino-3,3-dimethylbutanoyl)-4-hydroxy-N-(2-hydroxy-4-(4-methylthiazol-5-yl)benzyl)pyrrolidine-2-carboxamide

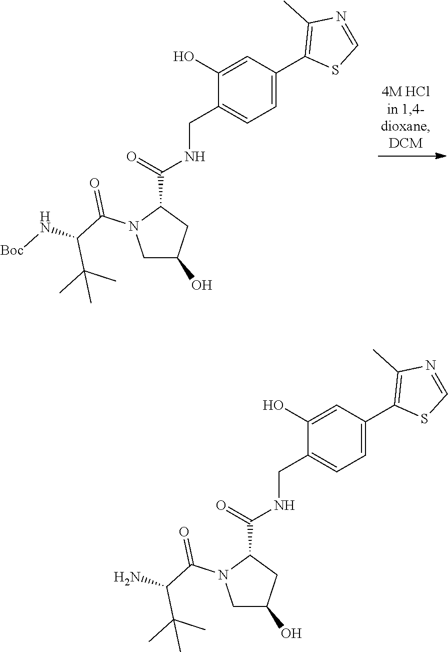 [SMILES: Cc1ncsc1-c1ccc(CNC(=O)[C@@H]2C[C@@H](O)CN2C(=O)[C@@H](NC(=O)OC(C)(C)C)C(C)(C)C)c(O)c1 ; Cc1ncsc1-c1ccc(CNC(=O)[C@@H]2C[C@@H](O)CN2C(=O)[C@@H](N)C(C)(C)C)c(O)c1]

A mixture of tert-butyl ((S)-1-((2S,4R)-4-hydroxy-2-((2-hydroxy-4-(4-methylthiazol-5-yl)benzyl)carbamoyl)pyrrolidin-1-yl)-3,3-dimethyl-1-oxobutan-2-yl)carbamate (0.60 g, 1.09 mmol, from step 3) and hydrochloric acid (4 M in 1,4-dioxane, 4 mL) in dichloromethane (4 mL) was stirred at 0° C. for 5 min then stirred at RT for 2 h. The progress of the reaction was monitored by TLC. After completion of the reaction, the reaction mixture was concentrated under reduced pressure to afford (2S,4R)-1-((S)-2-amino-3,3-dimethylbutanoyl)-4-hydroxy-N-(2-hydroxy-4-(4-methylthiazol-5-yl)benzyl)pyrrolidine-2-carboxamide (0.90 g, 93%, HCl salt) as a brown solid. LCMS: m/z 446.90 [M+H]⁺.

### Step 5: (2S,4R)-1-((S)-2-acetamido-3,3-dimethylbutanoyl)-4-hydroxy-N-(2-hydroxy-4-(4-methylthiazol-5-yl)benzyl)pyrrolidine-2-carboxamide

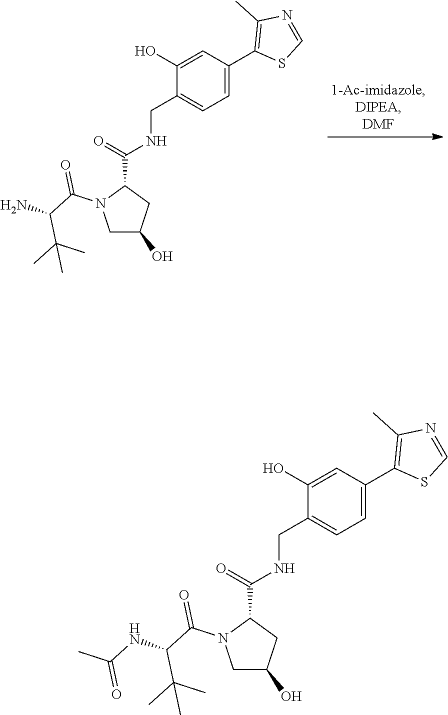 [SMILES: Cc1ncsc1-c1ccc(CNC(=O)[C@@H]2C[C@@H](O)CN2C(=O)[C@@H](N)C(C)(C)C)c(O)c1 ; CC(=O)N[C@H](C(=O)N1C[C@H](O)C[C@H]1C(=O)NCc1ccc(-c2scnc2C)cc1O)C(C)(C)C]

To a stirred solution of (2S,4R)-1-((S)-2-amino-3,3-dimethylbutanoyl)-4-hydroxy-N-(2-hydroxy-4-(4-methylthiazol-5-yl)benzyl)pyrrolidine-2-carboxamide (0.10 g, 0.20 mmol, from step 4) in DMF (3 mL) were added 1-acetylimidazole (0.05 g, 0.43 mmol) and N,N-diisopropylethylamine (0.196 mL, 1.12 mmol), respectively, and the reaction mixture was stirred at RT for 16 h. The progress of the reaction was monitored by TLC and LCMS. The reaction mixture was concentrated under reduced pressure to remove excess of DMF, diluted with water and extracted with ethyl acetate. The organic layer was dried over anhydrous sodium sulphate, filtered, and concentrated to afford (2S,4R)-1-((S)-2-acetamido-3,3-dimethylbutanoyl)-4-hydroxy-N-(2-hydroxy-4-(4-methylthiazol-5-yl)benzyl)pyrrolidine-2-carboxamide (0.08 g, 54%) as light brown semi solid. LCMS: m/z 489.30 [M+H]⁺.

### Step 6: tert-butyl (6-(2-(((2S,4R)-1-((S)-2-acetamido-3,3-dimethylbutanoyl)-4-hydroxypyrrolidine-2-carboxamido)methyl)-5-(4-methylthiazol-5-yl)phenoxy)hexyl)-carbamate

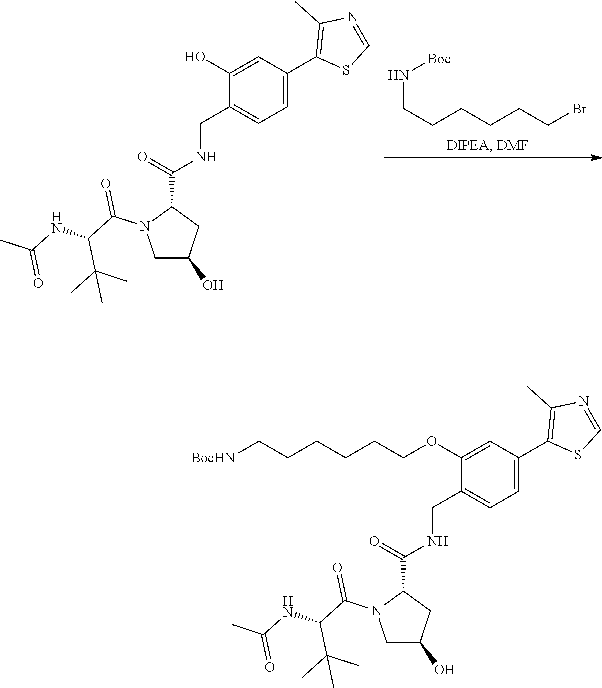

To a stirred solution of (2S,4R)-1-((S)-2-acetamido-3,3-dimethylbutanoyl)-4-hydroxy-N-(2-hydroxy-4-(4-methylthiazol-5-yl)benzyl)pyrrolidine-2-carboxamide (0.08 g, 0.16 mmol, from step 5) in DMF (2 mL, 25.8 mmol) were added tert-butyl (6-bromohexyl)carbamate (0.09 g, 0.32 mmol) and N,N-diisopropylethylamine (0.14 mL, 0.81 mmol), respectively, and the reaction mixture was stirred at 60° C. for 16 b. The progress of the reaction was monitored by TLC and LCMS. The reaction mixture was quenched with cold water and extracted with ethyl acetate. The organic layer was dried over anhydrous sodium sulphate, filtered, and concentrated under reduced pressure to afford tert-butyl (6-(2-(((2S,4R)-1-((S)-2-acetamido-3,3-dimethylbutanoyl)-4-hydroxypyrrolidine-2-carboxamido)methyl)-5-(4-methylthiazol-5-yl)phenoxy)hexyl)carbamate (0.07 g, 62%) as an off white solid. LCMS: m/z 688.30 [M+H]⁺.

### Step 7: (2S,4R)-1-((S)-2-acetamido-3,3-dimethylbutanoyl)-N-(2-((6-aminohexyl)oxy)-4-(4-methylthiazol-5-yl)benzyl)-4-hydroxypyrrolidine-2-carboxamide

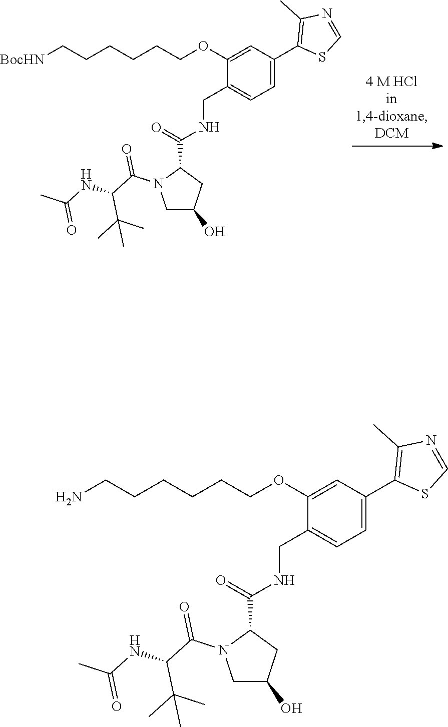 [SMILES: CC(=O)N[C@H](C(=O)N1C[C@H](O)C[C@H]1C(=O)NCc1ccc(-c2scnc2C)cc1OCCCCCCNC(=O)OC(C)(C)C)C(C)(C)C ; CC(=O)N[C@H](C(=O)N1C[C@H](O)C[C@H]1C(=O)NCc1ccc(-c2scnc2C)cc1OCCCCCCN)C(C)(C)C]

To a stirred solution of tert-butyl (6-(2-(((2S,4R)-1-((S)-2-acetamido-3,3-dimethylbutanoyl)-4-hydroxypyrrolidine-2-carboxamido)methyl)-5-(4-methylthiazol-5-yl)phenoxy)hexyl)carba-mate (0.07 g, 0.10 mmol, from step 6) in DCM was added hydrochloric acid (4 M in 1,4-dioxane, 1 mL) at 0° C., and the reaction mixture was stirred at RT for 2 h. The progress of the reaction was monitored by TLC. After the completion of the reaction, the reaction mixture was concentrated under reduced pressure and triturated with diethyl ether to afford (2S,4R)-1-((S)-2-acetamido-3,3-dimethylbutanoyl)-N-(2-((6-aminohexyl)oxy)-4-(4-methylthiazol-5-yl)benz-yl)-4-hydroxypyrrolidine-2-carboxamide (0.06 g, 100%, HCl salt) as an off-white solid. LCMS: m/z 588.10 [M+H]⁺.

### Step 8: (2S,4R)-1-((S)-2-acetamido-3,3-dimethylbutanoyl)-N-(2-((6-(4-((3R,5R)-5-((5-bromo-1-methyl-6-oxo-1,6-dihydropyridazin-4-yl)amino)-1-methylpiperidin-3-yl)benzamido) hexyl)oxy)-4-(4-methylthiazol-5-yl)benzyl)-4-hydroxypyrrolidine-2-carboxamide

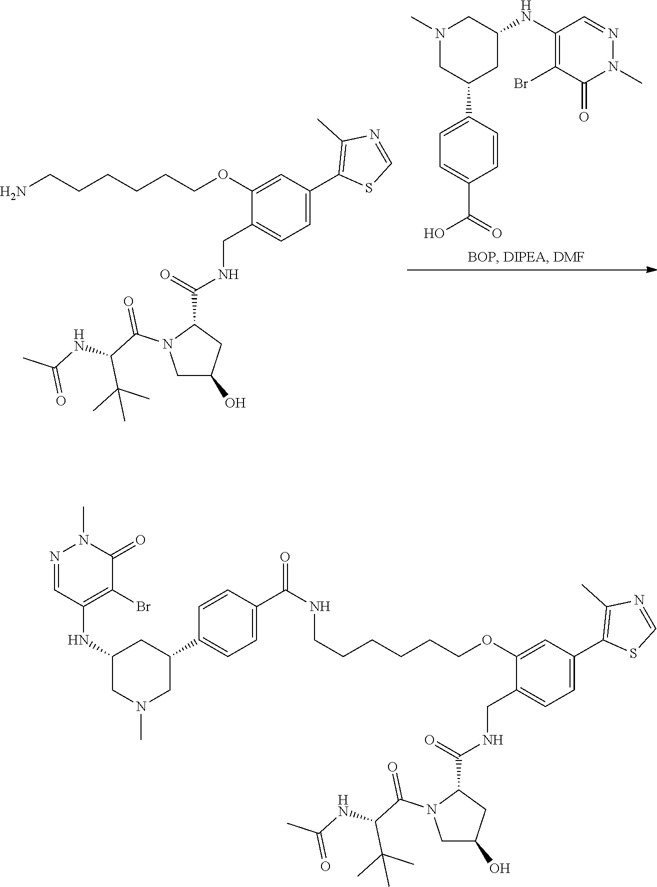

To a stirred solution of (2S,4R)-1-((S)-2-acetamido-3,3-dimethylbutanoyl)-N-(2-((6-aminohexyl)oxy)-4-(4-methylthiazol-5-yl)benzyl)-4-hydroxypyrrolidine-2-carboxamide (0.09 g, 0.16 mmol, from step 7) in DMF (2 mL) were added 4-((3R,5R)-5-((5-bromo-1-methyl-6-oxo-1,6-dihydropyridazin-4-yl)amino)-1-methylpiperidin-3-yl)benzoic acid (0.070 g, 0.16 mmol) and N,N-diisopropylethylamine (0.145 mL, 0.83 mmol) and the reaction mixture was stirred at RT for 10 min. To this was added BOP (0.11 g, 0.25 mmol) and the reaction mixture was stirred at RT for 4 h. The progress of the reaction was monitored by TLC and LCMS. The reaction mixture was diluted with ice-cold water and extracted with ethyl acetate. The organic layer was dried over anhydrous sodium sulphate, filtered, and concentrated under reduced pressure to obtain crude. The crude compound was purified by preparative HPLC [Column: X-BRIDGE C18 (250*30 mm) 5μ. Mobile Phase A: 10 mM ABC in H₂O, Mobile Phase B: 100% CAN] to afford (2S,4R)-1-((S)-2-acetamido-3,3-dimethylbutanoyl)-N-(2-((6-(4-((3R,5R)-5-((5-bromo-1-methyl-6-oxo-1,6-dihydropyridazin-4-yl)amino)-1-methylpiperi-din-3-yl)benzamido)hexyl)oxy)-4-(4-methylthiazol-5-yl)benzyl)-4-hydroxypyrrolidine-2-carboxamide (0.002 g, 1.3%) as a white solid. ¹H NMR (400 MHz, DMSO-d₆) δ=8.97 (s, 1H), 8.44-8.35 (m, 2H), 7.94 (d, J=9.6 Hz, 1H), 7.88 (s, 1H), 7.78 (d, J=8.4 Hz, 2H), 7.46 (d, J=8.0 Hz, 1H), 7.36-7.31 (m, 2H), 6.99 (d, J=1.2 Hz, 1H), 6.92-6.88 (m, 1H), 5.80 (d, J=9.2 Hz, 1H), 5.11 (d, J=3.6 Hz, 1H), 4.56-4.43 (m, 2H), 4.37-4.28 (m, 2H), 4.15 (dd, J=16.6, 5.2 Hz, 1H), 4.06-3.91 (m, 3H), 3.67-3.58 (m, 5H), 3.27-3.22 (m, 2H), 3.03-2.91 (m, 2H), 2.84 (d, J=10.8 Hz, 1H), 2.45 (s, 3H), 2.25 (s, 3H), 2.07-1.86 (m, 6H), 1.79-1.66 (m, 3H), 1.58-1.23 (m, 8H), 0.91 (s, 9H). LCMS: m/z 990.20 [M+H]⁺.

### Example 13: 3-(4-(9-(4-((3R,5R)-5-((5-bromo-1-methyl-6-oxo-1,6-dihydropyridazin-4-yl)amino)-1-cyclopropylpiperidin-3-yl)benzoyl)-3,9-diazaspiro[5.5]undecan-3-yl)phenyl)piperidine-2,6-dione (Compound 424)

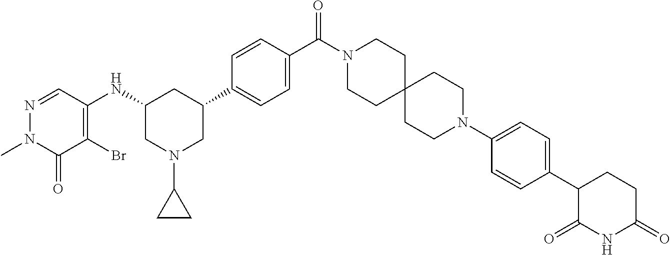 [SMILES: Cn1ncc(N[C@@H]2C[C@H](c3ccc(C(=O)N4CCC5(CC4)CCN(c4ccc(C6CCC(=O)NC6=O)cc4)CC5)cc3)CN(C3CC3)C2)c(Br)c1=O]

### Step 1: Ethyl 4-((3R,5R)-1-cyclopropyl-5-(picolinamido) piperidin-3-yl)benzoate

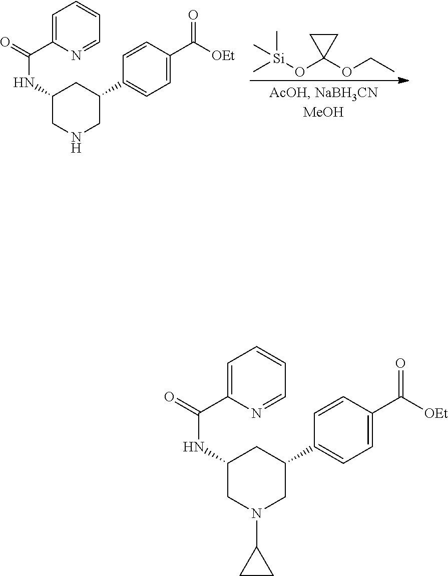 [SMILES: CCOC(=O)c1ccc([C@@H]2CNC[C@H](NC(=O)c3ccccn3)C2)cc1 ; CCOC1(O[Si](C)(C)C)CC1 ; CCOC(=O)c1ccc([C@H]2C[C@@H](NC(=O)c3ccccn3)CN(C3CC3)C2)cc1]

To a stirred solution of ethyl 4-[(3R,5R)-5-(pyridine-2-carbonylamino)-3-piperidyl]benzoate (1.00 g, 2.83 mmol) in methanol (10 mL), (1-ethoxycyclopropoxy)-trimethyl-silane (0.74 g, 4.24 mmol), acetic acid (0.25 g, 4.24 mmol), and sodium cyanoborohydride (0.37 g, 5.65 mmol) were added at 0° C. and was stirred at 60° C. for 16 h. The progress of the reaction was monitored by TLC. After completion of reaction, the reaction mixture was concentrated under reduced pressure, then reaction was quenched with NaHCO₃ and extracted with DCM. The combined organic layer was washed with water and brine, dried over anhydrous Na₂SO₄ and concentrated under reduced pressure to get crude which was purified by silica gel column chromatography using (80% EtOAc in Heptane) to afford ethyl 4-((3R,5R)-1-cyclopropyl-5-(picolinamido)piperidin-3-yl)benzoate (0.70 g, 62.90%) as liquid. LC-MS: m/z 394.10 [M+H]⁺.

### Step 2: Ethyl 4-[(3R,5R)-5-amino-1-cyclopropyl-3-piperidyl]benzoate

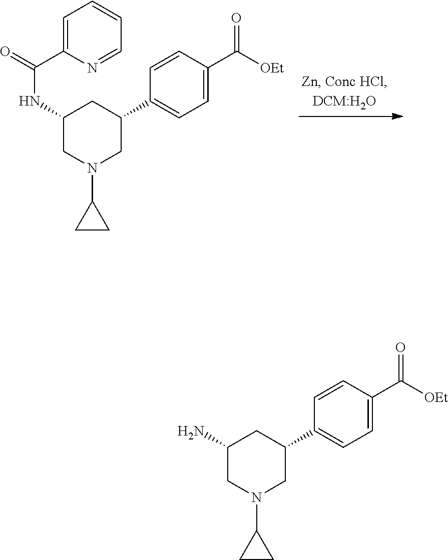 [SMILES: CCOC(=O)c1ccc([C@H]2C[C@@H](NC(=O)c3ccccn3)CN(C3CC3)C2)cc1 ; CCOC(=O)c1ccc([C@H]2C[C@@H](N)CN(C3CC3)C2)cc1]

To a stirred solution of ethyl 4-[(3R,5R)-1-cyclopropyl-5-(pyridine-2-carbonylamino)-3-piperidyl]benzoate (0.70 g, 1.77 mmol, from step 1) in dichloromethane (7 mL) and water (7 mL) was added concentrated hydrochloric acid (1.5 mL) at 0° C. and stirred at RT for 30 minutes, then Zinc (1.75 g, 26.7 mmol) was added at 0° C. and stirred for 10 min. The reaction mixture was stirred at RT for 16 h. The progress of the reaction was monitored by TLC. After completion of reaction, the reaction mixture was quench with 1M NaOH and extracted with DCM. The combined organic layer was washed with water and brine, dried over anhydrous Na₂SO₄ and concentrated under reduced pressure to afford ethyl 4-[(3R,5R)-5-amino-1-cyclopropyl-3-piperidyl]benzoate (300.00 g, 58.48%) as colourless liquid. LC-MS: m/z 289.10 [M+H]⁺.

### Step 3: Ethyl 4-[(3R,5R)-5-[(5-bromo-1-methyl-6-oxo-pyridazin-4-yl)amino]-1-cyclopropyl-3-piperidyl]benzoate

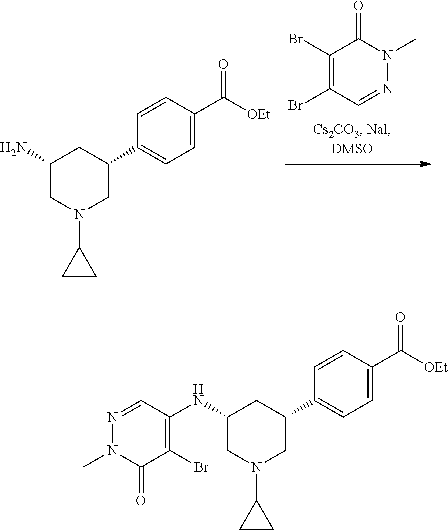

To a stirred solution of ethyl 4-[(3R,5R)-5-amino-1-cyclopropyl-3-piperidyl]benzoate (0.40 g, 1.00 mmol, from step 2) and 4,5-dibromo-2-methyl-pyridazin-3-one (0.45 g, 1.70 mmol), in DMSO (8 mL), were added cesium carbonate (0.90 g, 3.00 mmol) and sodium iodide (0.18 g, 1.2 mmol) at RT. The reaction mixture was stirred in a sealed tube at 110° C. for 16 h. The progress of the reaction was monitored by TLC. After completion of reaction, reaction mixture was purified by silica gel column chromatography using (80% EtOAc in Heptane) to afford ethyl 4-[(3R,5R)-5-[(5-bromo-1-methyl-6-oxo-pyridazin-4-yl)amino]-1-cyclopropyl-3-piperidyl]-benzoate (0.15 g, 20%) as pale brown semi solid. LC-MS: m/z 475.10 and 477.10 [M+H]⁺.

### Step 4: 4-[(3R,5R)-5-[(5-bromo-1-methyl-6-oxo-pyridazin-4-yl)amino]-1-cyclopropyl-3-piperidyl]benzoic acid

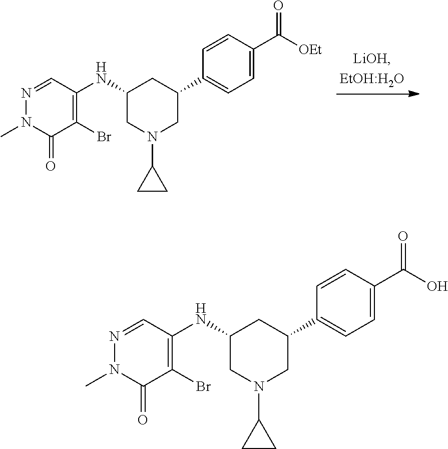 [SMILES: CCOC(=O)c1ccc([C@H]2C[C@@H](Nc3cnn(C)c(=O)c3Br)CN(C3CC3)C2)cc1 ; Cn1ncc(N[C@@H]2C[C@H](c3ccc(C(=O)O)cc3)CN(C3CC3)C2)c(Br)c1=O]

To a stirred solution of ethyl 4-[(3R,5R)-5-[(5-bromo-1-methyl-6-oxo-pyridazin-4-yl)amino]-1-cyclopropyl-3-piperidyl]benzoate (0.15 g, 0.32 mmol, from step 3) in ethanol (4 mL) and water (0.2 mL) was added lithium hydroxide (0.01 g, 0.49 mmol) and the reaction mixture was refluxed for 2 h. The progress of the reaction was monitored by TLC. Then reaction mixture was acidified with dilute HCl and concentrated under reduced pressure to obtain crude product. The crude obtained was triturated with diethyl ether to afford 4-[(3R,5R)-5-[(5-bromo-1-methyl-6-oxo-pyridazin-4-yl)amino]-1-cyclopropyl-3-piperidyl]benzoic acid (0.12 g, 85%) as pale brown solid. LC-MS: m/z 447.29 and 449.20 [M+H]⁺.

### Step in 3-(4-(9-(4-((3R,5R)-5-((5-bromo-1-methyl-6-oxo-1,6-dihydropyridazin-4-yl)amino)-1-cyclopropylpiperidin-3-yl)benzoyl)-3,9-diazaspiro[5.5]undecan-3-yl) phen-yl)piperidine-2,6-dione

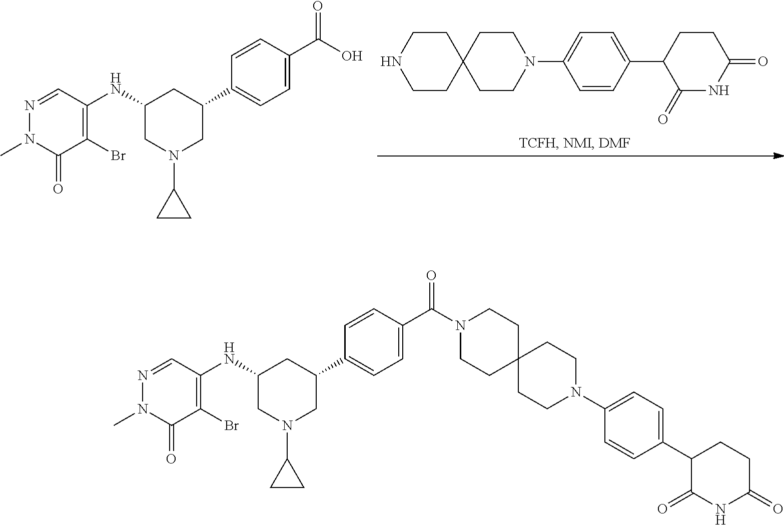 [SMILES: Cn1ncc(N[C@@H]2C[C@H](c3ccc(C(=O)O)cc3)CN(C3CC3)C2)c(Br)c1=O ; O=C1CCC(c2ccc(N3CCC4(CCNCC4)CC3)cc2)C(=O)N1 ; Cn1ncc(N[C@@H]2C[C@H](c3ccc(C(=O)N4CCC5(CC4)CCN(c4ccc(C6CCC(=O)NC6=O)cc4)CC5)cc3)CN(C3CC3)C2)c(Br)c1=O]

To a stirred solution of 4-[(3R,5R)-5-[(5-bromo-1-methyl-6-oxo-pyridazin-4-yl)amino]-1-cyclopropyl-3-piperidyl]benzoic acid (0.08 g, 0.17 mmol, from step 4) in DMF, was added (4 mL) 3-[4-(3,9-diazaspiro[5.5]undecan-3-yl)phenyl]piperidine-2,6-dione (0.07 g, 0.21 mmol) followed by 1-methylimidazole (0.07 g, 0.89 mmol) and the reaction mixture was stirred at RT for 10 min. To this was added N,N,N′,N′-tetramethylchloroformamidinium hexafluorophosphate (0.07 g, 0.26 mmol) and the reaction mixture was stirred at RT for 2 h. The progress of the reaction was monitored by TLC and LCMS. After completion of reaction, ice cold water was added and extracted with 10% of methanol in DCM. The combined organic layer was dried over Na₂SO₄, filtered, and concentrated under reduced pressure to obtained crude. The crude was purified by preparative HPLC to afford 3-(4-(9-(4-((3R,5R)-5-((5-bromo-1-methyl-6-oxo-1,6-dihydropyridazin-4-yl)amino)-1-cyclopropylpiperidin-3-yl)benzoyl)-3,9-diazaspiro[5.5]undecan-3-yl)phenyl)piperidine-2,6-dione (0.01 g, 11.96%) as an off white solid. ¹H NMR (400 MHz, DMSO-d₆) δ=10.75 (s, 1H), 7.85 (s, 1H), 7.33 (s, 4H), 7.03 (d, J=8.8 Hz, 2H), 6.89 (d, J=8.9 Hz, 2H), 5.82 (d, J=8.9 Hz, 1H), 3.92-3.83 (m, 1H), 3.71 (dd, J=4.9, 10.9 Hz, 1H), 3.67-3.54 (m, 5H), 3.28-3.26 (m, 1H), 3.19-3.06 (m, 5H), 3.04-2.97 (m, 1H), 2.96-2.86 (m, 1H), 2.65-2.57 (m, 1H), 2.47-2.38 (m, 2H), 2.30-2.20 (m, 2H), 2.16-1.97 (m, 3H), 1.82-1.70 (m, 2H), 1.65-1.56 (m, 4H), 1.55-1.38 (m, 4H), 0.48-0.31 (m, 4H). LC-MS: m/z 770.50 and 772.50 [M+H]⁺.

### Example 14:3-[4-[3-[4-[(3R,5R)-5-[(5-bromo-1-methyl-6-oxo-pyridazin-4-yl)amino]-1-(cyclopropylmethyl)-3-piperidyl]benzoyl]-3,9-diazaspiro[5.5]undecan-9-yl]phenyl]piperidine-2,6-dione (Compound 425)

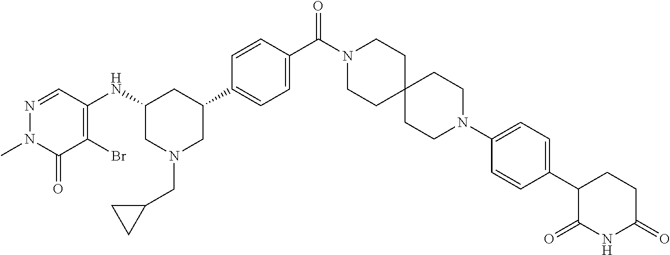 [SMILES: Cn1ncc(N[C@@H]2C[C@H](c3ccc(C(=O)N4CCC5(CC4)CCN(c4ccc(C6CCC(=O)NC6=O)cc4)CC5)cc3)CN(CC3CC3)C2)c(Br)c1=O]

### Step 1: tert-butyl (3R,5R)-3-(4-(ethoxycarbonyl)phenyl)-5-(picolinamido)piperidine-1-carboxylate

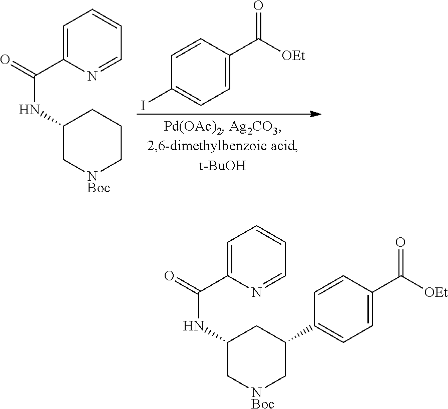

To a stirred solution of tert-butyl (R)-3-(picolinamido)piperidine-1-carboxylate (16.00 g, 52.39 mmol) and ethyl 4-iodobenzoate (72.00 g, 260.80 mmol) in tert-butanol (160 mL) was added silver carbonate (14.59 g, 52.38 mmol) at RT and the reaction mixture was purged with argon for 20 min. To the resulting reaction mixture, 2,4-dimethylbenzoic acid (2.00 g, 13.05 mmol) and palladium (II) acetate (1.24 g, 5.25 mmol) were added, and the reaction mixture was again purged with argon for 20 minutes. The reaction mixture was stirred in a sealed tube at 120° C. for 16 h. The progress of the reaction was monitored by TLC. After completion of reaction, the reaction mixture was filtered through celite. The filtrate was concentrated under reduced pressure to get crude which was purified by column chromatography by using (50% EtOAc in heptane) to afford tert-butyl (3R,5R)-3-(4-(ethoxycarbonyl)phenyl)-5-(picolinamido)piperidine-1-carboxylate (7.20 g, 30%) as an off white solid. LCMS: m/z 454.10 [M+H]⁺.

### Step 2: Ethyl 4-((3R,5R)-5-(picolinamido)piperidin-3-yl)benzoate

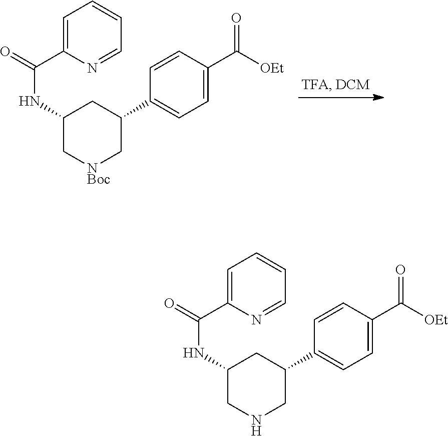 [SMILES: CCOC(=O)c1ccc([C@H]2C[C@@H](NC(=O)c3ccccn3)CN(C(=O)OC(C)(C)C)C2)cc1 ; CCOC(=O)c1ccc([C@@H]2CNC[C@H](NC(=O)c3ccccn3)C2)cc1]

To a stirred solution of tert-butyl (3R,5R)-3-(4-(ethoxycarbonyl)phenyl)-5-(picolinamido)piperidine-1-carboxylate (4.00 g, 8.82 mmol, from step 1) in DCM (40 mL). The reaction mixture was stirred in a sealed tube at 0° C. for 10 min. Then trifluoroacetic acid (TFA) (12 mL) was added at 0° C. for 20 min and was stirred at RT for 50 min. The progress of the reaction was monitored by TLC. After completion of reaction, the reaction mixture was concentrated under reduced pressure. The crude obtained was purified by trituration in ether to afford ethyl 4-((3R,5R)-5-(picolinamido)piperidin-3-yl)benzoate (3.00 g, 96.24%) as an off white solid. LCMS: m/z 354.20 [M+H]⁺.

### Step 3: Ethyl 4-[(3R,5R)-1-(cyclopropylmethyl)-5-(pyridine-2-carbonylamino)-3-piperidyl]benzoate

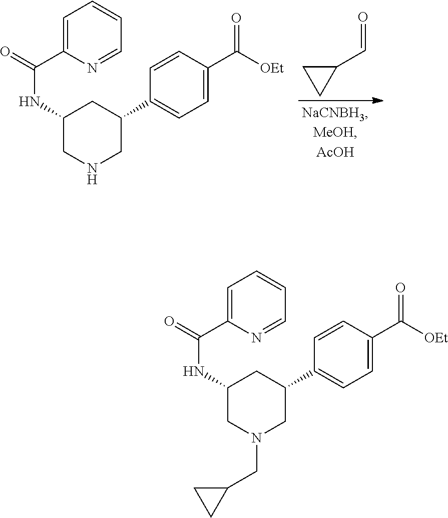

To a stirred solution of ethyl 4-((3R,5R)-5-(picolinamido)piperidin-3-yl)benzoate (2.70 g, 7.60 mmol, from step 2) in methanol (30 mL), cyclopropane carbaldehyde (0.80 g, 11.41 mmol) was added and reaction mixture was stirred at RT for 25 min, then acetic acid (0.69 g, 11.50 mmol) was added, and the reaction mixture was stirred at RT for 30 min. To the resulting reaction mixture, sodium cyanoborohydride (1.00 g, 15.10 mmol) was added and the reaction mixture was stirred at RT for 12 h. The progress of the reaction was monitored by TLC. After completion of reaction, the reaction mixture was quenched with water and extracted with DCM. The combined organic layer was washed with water and brine, dried over anhydrous Na₂SO₄ and concentrated under reduced pressure to get the crude which was purified by silica gel column chromatography using (30% MeOH in DCM) to afford ethyl 4-[(3R,5R)-1-(cyclopropylmethyl)-5-(pyridine-2-carbonylamino)-3-piperidyl]benzoate (0.98 g, 31%) as an colourless liquid. LCMS: m/z 408.22 [M+H]⁺.

### Step 4: Ethyl 4-[(3R,5R)-5-amino-1-(cyclopropylmethyl)-3-piperidyl]benzoate

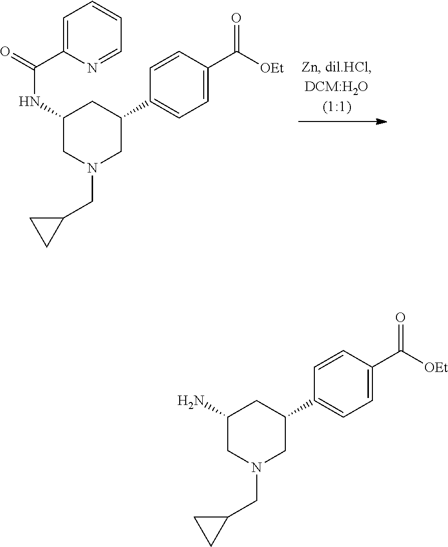 [SMILES: CCOC(=O)c1ccc([C@H]2C[C@@H](NC(=O)c3ccccn3)CN(CC3CC3)C2)cc1 ; CCOC(=O)c1ccc([C@H]2C[C@@H](N)CN(CC3CC3)C2)cc1]

To a stirred solution of ethyl 4-[(3R,5R)-1-(cyclopropylmethyl)-5-(pyridine-2-carbonylamino)-3-piperidyl]benzoate (1.00 g, 2.45 mmol, from step 3) in DCM (5 mL), water (5 mL) and diluted hydrochloric acid (5 mL) were added at 0° C. and stirred at RT for 30 minutes, then zinc (2.41 g, 36.80 mmol) was added at 0° C. and was stirred at RT for 16 h. The progress of the reaction was monitored by TLC. After completion of reaction, the reaction mixture was concentrated under reduced pressure, then reaction mixture was quenched with 1M NaOH and extracted with DCM. The obtained crude was purified by reverse phase purification using 0.1% formic acid in acetonitrile and water (15% CAN in water) to afford ethyl 4-[(3R,5R)-5-amino-1-(cyclopropylmethyl)-3-piperidyl]benzoate (0.50 g, 70%) as liquid. LCMS: m/z 303.08 [M+H]⁺.

### Step 5: Ethyl 4-[(3R,5R)-5-[(5-bromo-1-methyl-6-oxo-pyridazin-4-yl)amino]-1-(cyclopropylmethyl)-3-piperidyl]benzoate

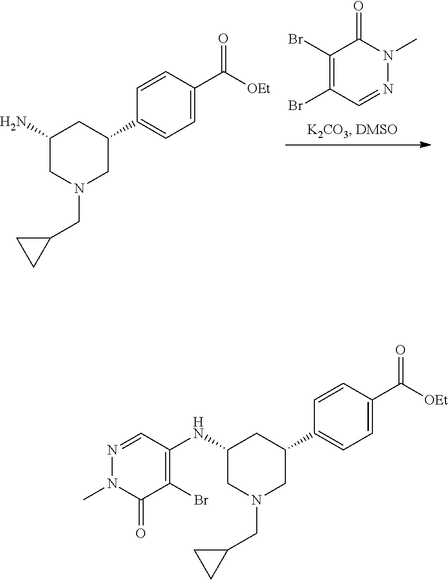

To a stirred solution of ethyl 4-[(3R,5R)-5-amino-1-(cyclopropylmethyl)-3-piperidyl]benzoate (0.50 g, 1.65 mmol, from step 4) and dimethyl sulfoxide (10 mL), 4,5-dibromo-2-methyl-pyridazin-3-one (0.53 g, 1.98 mmol) and potassium carbonate (0.91 g, 6.61 mmol) were added at RT. The reaction mixture was stirred in a microwave at 110° C. for 2 h. The progress of the reaction was monitored by TLC. After completion of reaction, the reaction mixture was quenched with cold water and extracted with ethyl acetate. The combined organic layer was washed with water and brine, dried over anhydrous Na₂SO₄ and concentrated under reduced pressure to afford crude product which was purified by silica gel column chromatography using (5% MeOH in DCM) to afford ethyl 4-[(3R,5R)-5-[(5-bromo-1-methyl-6-oxo-pyridazin-4-yl)amino]-1-(cyclopropylmethyl)-3-piperidyl]benzoate as an off white solid. LCMS: m/z 489.11 and 491.11 [M+H]⁺.

### Step 6: 4-[(3R,5R)-5-[(5-bromo-1-methyl-6-oxo-pyridazin-4-yl)amino]-1-(cyclopropyl-methyl)-3-piperidyl]benzoic acid

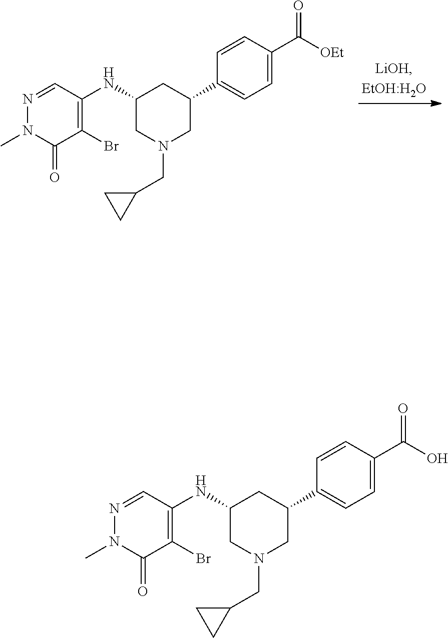 [SMILES: CCOC(=O)c1ccc([C@H]2C[C@@H](Nc3cnn(C)c(=O)c3Br)CN(CC3CC3)C2)cc1 ; Cn1ncc(N[C@@H]2C[C@H](c3ccc(C(=O)O)cc3)CN(CC3CC3)C2)c(Br)c1=O]

To a stirred solution of ethyl 4-[(3R,5R)-5-[(5-bromo-1-methyl-6-oxo-pyridazin-4-yl)amino]-1-(cyclopropylmethyl)-3-piperidyl]benzoate (0.80 g, 0.16 mmol, from step 5) in ethanol (4 mL) and water (0.5 mL), lithium hydroxide (0.01 g, 0.25 mmol) was added at RT. The reaction mixture was stirred at 80° C. for 2 h. The progress of the reaction was monitored by TLC. After completion of reaction, reaction mixture was concentrated under reduced pressure and was added 1N HCl. The crude obtained was filtered, washed with diethyl ether and dried under reduced pressure to afford 4-[(3R,5R)-5-[(5-bromo-1-methyl-6-oxo-pyridazin-4-yl)amino]-1-(cyclopropylmethyl)-3-piperidyl]benzoic acid (0.07 g, 92.81%) as an off white solid. LCMS: m/z 461.19 and 463.19 [M+H]⁺.

### Step 7: 3-[4-[3-[4-[(3R,5R)-5-[(5-bromo-1-methyl-6-oxo-pyridazin-4-yl)amino]-1-(cyclopropylmethyl)-3-piperidyl]benzoyl]-3,9-diazaspiro[5.5]undecan-9-yl]phenyl]-piperidine-2,6-dione

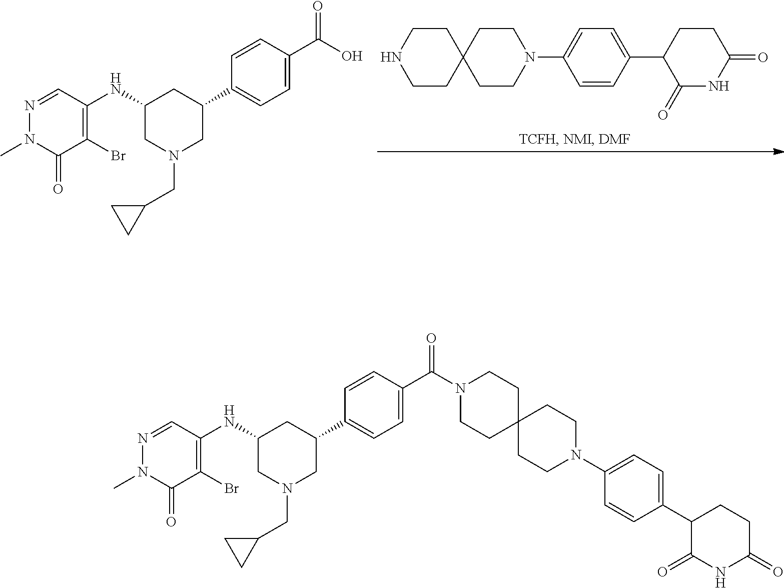 [SMILES: Cn1ncc(N[C@@H]2C[C@H](c3ccc(C(=O)O)cc3)CN(CC3CC3)C2)c(Br)c1=O ; O=C1CCC(c2ccc(N3CCC4(CCNCC4)CC3)cc2)C(=O)N1 ; Cn1ncc(N[C@@H]2C[C@H](c3ccc(C(=O)N4CCC5(CC4)CCN(c4ccc(C6CCC(=O)NC6=O)cc4)CC5)cc3)CN(CC3CC3)C2)c(Br)c1=O]

To the stirred solution of 3-[4-(3,9-diazaspiro[5.5]undecan-3~yl)phenyl]piperidine-2,6-dione (0.08 g, 0.18 mmol, TFA salt) in a DMF (1 mL) were added 1-methylimidazole (0.06 mL, 0.70 mmol) followed by 4-[(3R,5R)-5-[(5-bromo-1-methyl-6-oxo-pyridazin-4-yl)amino]-1-(cyclopropylmethyl)-3-piperidyl]benzoic acid (0.07 g, 0.15 mmol, from step 6) at RT. To this N,N,N′,N′-tetramethylchloroformamidinium hexafluorophosphate (0.06 g, 0.22 mmol) was added and reaction stirred at RT for 16 h. The progress of the reaction was monitored by TLC. After completion, the reaction mixture was quenched in ice-water. The solid formed was filtered and dried concentrated under reduced pressure to afford crude product. The crude obtained was purified by preparative HPLC to afford 3-[4-[3-[4-[(3R,5R)-5-[(5-bromo-1-methyl-6-oxo-pyridazin-4-yl)amino]-1-(cyclopropylmethyl)-3-piperidyl]benzoyl]-3,9-diazaspiro[5.5]undecan-9-yl]phenyl]piperidine-2,6-dione (0.01 g, 16.40%) as an off white solid. ¹H NMR (400 MHz, DMSO-d₆) δ=10.75 (s, 1H), 7.87 (s, 1H), 7.33 (s, 4H), 7.03 (d, J=8.8 Hz, 2H), 6.89 (d, J=8.8 Hz, 2H), 5.81 (d, J=9.0 Hz, 1H), 4.02-3.90 (m, 1H), 3.71 (dd, J=4.8, 10.8 Hz, 1H), 3.67-3.56 (m, 5H), 3.19-3.08 (m, 5H), 3.06-2.96 (m, 2H), 2.69-2.58 (m, 2H), 2.47-2.39 (m, 2H), 2.34-2.29 (m, 2H), 2.18-1.96 (m, 5H), 1.75 (q, J=11.9 Hz, 1H), 1.66-1.58 (m, 4H), 1.55-1.39 (m, 4H), 0.91-0.83 (m, 1H), 0.49-0.42 (m, 2H), 0.10-0.05 (m, 2H). LC-MS: m/z 784.52 and 786.49 [M+H]⁺.

### Example 15: 3-(4-(9-(4-((1S,2S)-1-(dimethylamino)-2-((3-methyl-[1,2,4]triazolo[3,4-a]phthalazin-6-yl)amino)propyl)benzoyl)-3,9-diazaspiro[5.5]undecan-3-yl)phenyl)piperidine-2,6-dione (Compound 426)

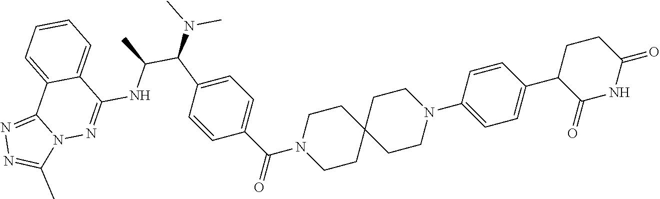 [SMILES: Cc1nnc2c3ccccc3c(N[C@@H](C)[C@H](c3ccc(C(=O)N4CCC5(CC4)CCN(c4ccc(C6CCC(=O)NC6=O)cc4)CC5)cc3)N(C)C)nn12]

### Step 1: Methyl 4-(2-nitroprop-1-en-1-yl)benzoate

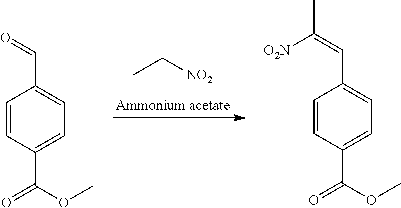

To a stirred solution of methyl 4-formylbenzoate (20.00 g, 121.83 mmol) in nitroethane (80 mL) was added ammonium acetate (10.00 g, 124.54 mmol) at RT. Reaction mixture was stirred at 120° C. for 16 h. Progress of reaction was monitored by TLC and LCMS. After completion, the reaction mixture was extracted with ethyl acetate. The organic layer was dried over anhydrous Na₂SO₄ and concentrated under reduced pressure to afford methyl 4-(2-nitroprop-1-en-1-yl)benzoate (18.00 g, 66.78%) as yellow solid; which was used as such for the next reaction. ¹H NMR (400 MHz, CDCl₃) δ=8.14-8.07 (m, 3H), 7.49 (d, J=8.8 Hz, 2H), 3.95-3.92 (m, 3H), 2.49-2.42 (m, 3H).

### Step 2: Methyl 4-((1S,2S)-1-(dimethylamino)-2-nitropropyl)benzoate

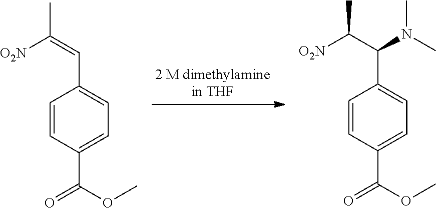 [SMILES: COC(=O)c1ccc(/C=C(/C)[N+](=O)[O-])cc1 ; COC(=O)c1ccc([C@@H]([C@H](C)[N+](=O)[O-])N(C)C)cc1]

To a stirred solution of methyl 4-(2-nitroprop-1-en-1-yl)benzoate (2.00 g, 9.04 mmol, from step 1) in tetrahydrofuran (15 mL) was added 2M solution of dimethylamine (24 mL) in THF at RT. The reaction mixture was stirred at RT for 16 h. The progress of reaction was monitored by TLC and LCMS. After completion of reaction, reaction mixture was concentrated under reduced pressure to afford methyl 4-((1S,2S)-1-(dimethylamino)-2-nitropropyl)benzoate (1.80 g, 75%) as orange solid; which was used as such for the next reaction. LC-MS: m/z 267.0 [M+H]⁺.

### Step 3: Methyl 4-((1S,2S)-2-amino-1-(dimethylamino)propyl)benzoate

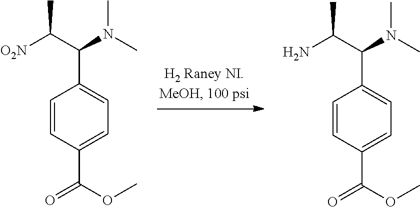 [SMILES: COC(=O)c1ccc([C@@H]([C@H](C)[N+](=O)[O-])N(C)C)cc1 ; COC(=O)c1ccc([C@@H]([C@H](C)N)N(C)C)cc1]

To a stirred solution of methyl 4-((1S,2S)-1-(dimethylamino)-2-nitropropyl)benzoate (1.00 g, 3.75 mmol, from step 2) in methanol (30 mL) was added raney nickel (0.50 g) at RT. The reaction mixture was stirred at RT under H₂ gas at 100 psi for 16 h. Progress of reaction was monitored by TLC and LCMS. After completion, reaction mixture was filtered through celite. Filtrate was concentrated under reduced pressure to get crude. The crude was purified by silica gel column chromatography using 5% NH₃ solution in 10% MeOH in DCM to afford methyl 4-((1S,2S)-2-amino-1-(dimethylamino)propyl)benzoate (0.25 g, 28.17%) as colorless liquid. LC-MS: m/z 237.20 [M+H]⁺.

### Step 4: Methyl 4-((1S,2S)-1-(dimethylamino)-2-((3-methyl-[1,2,4]triazolo[3,4-a]phthalazin-6-yl)amino)propyl)benzoate

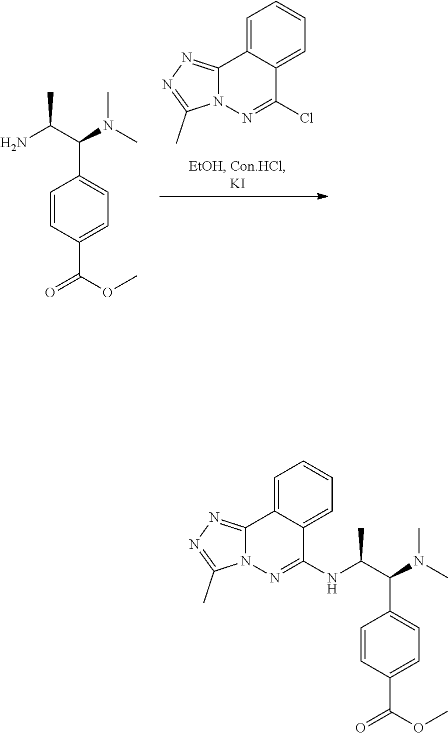

To a stirred solution of methyl 4-((1S,2S)-2-amino-1-(dimethylamino)propyl)benzoate (0.25 g, 1.05 mmol, from step 3) in ethanol (12 mL), was added hydrochloric acid (0.01 g, 0.27 mmol) followed by 6-chloro-3-methyl-[1,2,4]triazolo[3,4-a]phthalazine (0.12 g, 0.54 mmol) and potassium iodide (0.01 g, 0.05 mmol) at RT. The reaction mixture was stirred at 90° C. for 72 h. Progress of the reaction was monitored by TLC and LCMS. After completion of reaction, the reaction mixture was concentrated under reduced pressure to get crude, which was purified by silica gel column chromatography using 4% MeOH in DCM to afford methyl 4-((1S,2S)-1-(dimethylamino)-2-((3-methyl-[1,2,4]triazolo[3,4-a]phthalazin-6-yl)amino)propyl)benzoate (0.05 g, 21.77%) as yellow solid. LC-MS: m/z 419.37 [M+H]⁺.

### Step 5: 4-((1S,2S)-1-(dimethylamino)-2-((3-methyl-[1,2,4]triazolo[3,4-a]phthalazin-6-yl)amino)propyl)benzoic acid

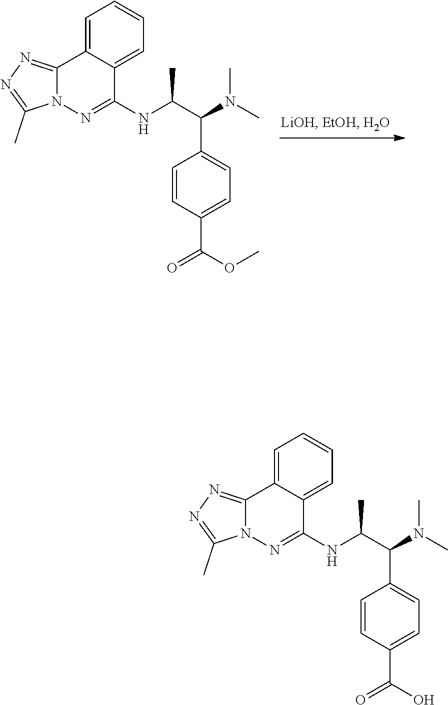 [SMILES: COC(=O)c1ccc([C@@H]([C@H](C)Nc2nn3c(C)nnc3c3ccccc23)N(C)C)cc1 ; Cc1nnc2c3ccccc3c(N[C@@H](C)[C@H](c3ccc(C(=O)O)cc3)N(C)C)nn12]

To a stirred solution of methyl 4-((1S,2S)-1-(dimethylamino)-2-((3-methyl-[1,2,4]triazolo[3,4-a]phthalazin-6-yl)amino)propyl)benzoate (0.05 g, 0.11 mmol, from step 4) in ethanol (3 mL) and water (0.5 mL) was added lithium hydroxide (0.01 g, 0.30 mmol) at RT and the reaction mixture was stirred at 80° C. for 2 h. The progress of the reaction was monitored by TLC. After completion of reaction, the reaction mixture was concentrated under reduced pressure and then 1N HCl was added and further concentrated under reduced pressure to afford 4-((1S,2S)-1-(dimethylamino)-2-((3-methyl-[1,2,4]triazolo[3,4-a]phthalazin-6-yl)amino)propyl)benzoic acid (0.04 g, 82.77%) as brown solid. LC-MS: m/z 405.37 [M+H]⁺.

### Step 6: 3~ (4-(9-(4-((1S,2S)-1-(dimethylamino)-2-((3-methyl-[1,2,4]triazolo[3,4-a]phthalazin-6-yl)amino)propyl)benzoyl)-3,9-diazaspiro[5.5]undecan-3-yl)phenyl)piperidine-2,6-dione

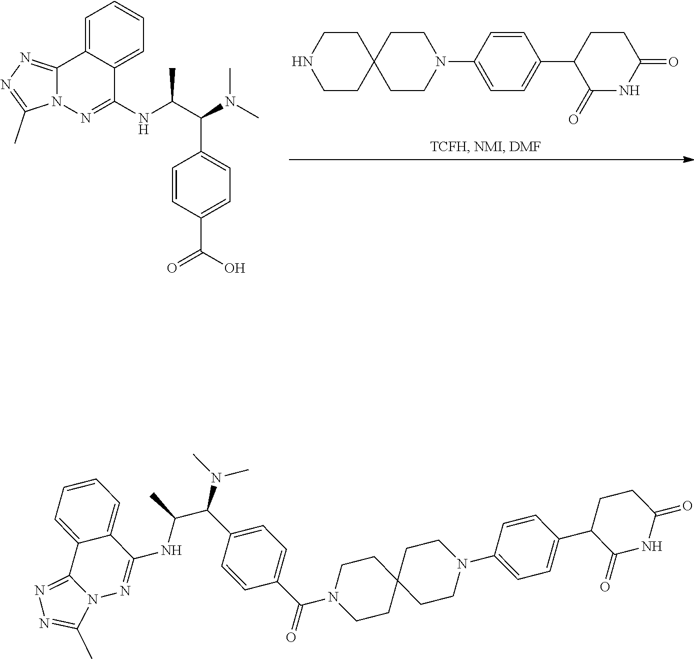 [SMILES: Cc1nnc2c3ccccc3c(N[C@@H](C)[C@H](c3ccc(C(=O)O)cc3)N(C)C)nn12 ; O=C1CCC(c2ccc(N3CCC4(CCNCC4)CC3)cc2)C(=O)N1 ; Cc1nnc2c3ccccc3c(N[C@@H](C)[C@H](c3ccc(C(=O)N4CCC5(CC4)CCN(c4ccc(C6CCC(=O)NC6=O)cc4)CC5)cc3)N(C)C)nn12]

To a stirred solution of 3-[4-(3,9-diazaspiro[5.5]undecan-3-yl)phenyl]piperidine-2,6-dione (0.04 g, 0.08 mmol, from step 5) in DMF (3 mL) was added 1-methylimidazole (0.04 g, 0.57 mmol) at RT. The reaction mixture was stirred at RT for 10 min. To this were added 4-[(1~{S},2~{S})-1-(dimethylamino)-2-[(3-methyl-[1,2,4]triazolo[3,4-a]phthalazin-6-yl)amino]propyl]benzoic acid (0.04 g, 0.09 mmol) followed by N,N,N′,N′-tetramethylchloroformamidinium hexafluorophosphate (0.05 g, 0.17 mmol) and the reaction mixture was stirred at RT for 16 h. The progress of the reaction was monitored by TLC and LCMS. After completion of reaction, the reaction mixture was diluted with ice-cold water and extracted with DCM. The combined organic layer was dried over anhydrous Na₂SO₄ and concentrated under reduced pressure to obtain crude product. The crude was purified by preparative HPLC to afford 3-(4-(9-(4-((1S,2S)-1-(dimethylamino)-2-((3-methyl-[1,2,4]triazolo[3,4-a]phthalazin-6-yl)amino)propyl)benzoyl)-3,9-diazaspiro[5.5]undecan-3-yl)phenyl)piperidine-2,6-dione (0.003 g) as an off white solid. ¹H NMR (400 MHz, DMSO-d₆) δ=10.75 (s, 1H), 8.41 (dd, J=1.1, 7.9 Hz, 1H), 8.30 (d, J=8.1 Hz, 1H), 7.98-7.90 (m, 1H), 7.86-7.78 (m, 1H), 7.41-7.33 (m, 2H), 7.28 (d, J=8.1 Hz, 2H), 7.08-7.00 (m, 3H), 6.89 (d, J=8.9 Hz, 2H), 4.81-4.71 (m, 1H), 3.80-3.69 (m, 2H), 3.67-3.55 (m, 2H), 3.21-3.09 (m, 4H), 2.64-2.61 (m, 3H), 2.47-2.42 (m, 4H), 2.15 (s, 6H), 2.11-2.07 (m, 1H), 2.04-1.97 (m, 1H), 1.66-1.58 (m, 4H), 1.56-1.42 (m, 4H), 1.13 (d, J=6.5 Hz, 3H). LC-MS: m/z 728.20 [M+H]⁺.

### Example 16: 3-(4-(9-(4-((3R,5R)-1-methyl-5-((3-methyl-4-oxo-4,5-dihydro-3H-pyrrolo[3,2-d]pyrimidin-2-yl)amino)piperidin-3-yl)benzoyl)-3,9-diazaspiro[5.5]undecan-3-yl)phenyl)piperidine-2,6-dione (Compound 427)

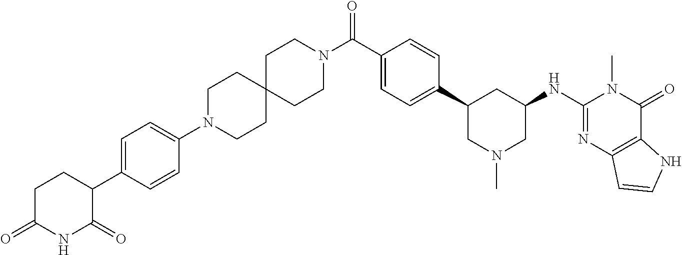 [SMILES: CN1C[C@H](Nc2nc3cc[nH]c3c(=O)n2C)C[C@H](c2ccc(C(=O)N3CCC4(CC3)CCN(c3ccc(C5CCC(=O)NC5=O)cc3)CC4)cc2)C1]

### Step 1: Ethyl 4-((3R,5R)-1-methyl-5-((3-methyl-4-oxo-4,5-dihydro-3H-pyrrolo[3,2-d]pyrimidin-2-yl)amino)piperidin-3-yl)benzoate

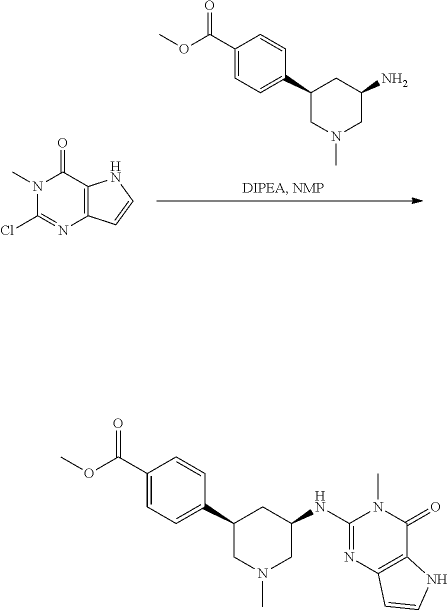 [SMILES: Cn1c(Cl)nc2cc[nH]c2c1=O ; COC(=O)c1ccc([C@H]2C[C@@H](N)CN(C)C2)cc1 ; COC(=O)c1ccc([C@H]2C[C@@H](Nc3nc4cc[nH]c4c(=O)n3C)CN(C)C2)cc1]

To a stirred solution of 2-chloro-3-methyl-3,5-dihydro-4H-pyrrolo[3,2-d]pyrimidin-4-one (0.07 g, 0.40 mmol,) in NMP (3.00 ml) was added N,N-diisopropylethylamine (0.20 mL, 1.14 mmol) and methyl 4-((3R,5R)-5-amino-1-methylpiperidin-3-yl)benzoate (0.07 g, 0.28 mmol, from step 5 of general intermediate 23) at RT, and the reaction mixture was stirred at 150° C. for 2 h. The progress of the reaction was monitored by TLC & LCMS. After completion of the reaction, the reaction mixture was diluted with ethyl acetate and washed with water. The combined organic layer was dried over anhydrous sodium sulphate, filtered, and concentrated under vacuum to obtain crude product. The crude was purified by column chromatography (silica gel, 230-400 mesh) using 5% methanol in DCM as eluent to afford ethyl 4-((3R,5R)-1-methyl-5-((3-methyl-4-oxo-4,5-dihydro-3H-pyrrolo[3,2-d]pyrimidin-2-yl)amino)piperidin-3-yl)benzoate (0.07 g, 59.81%) as brown solid. LCMS: m/z 410.19 [M+H]⁺.

### Step 2: 4-((3R,5R)-1-methyl-5-((3-methyl-4-oxo-4,5-dihydro-3H-pyrrolo[3,2-d]pyrimidin-2-yl)amino)piperidin-3-yl)benzoic acid

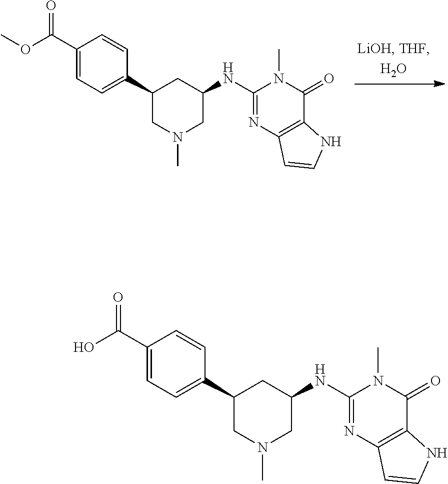 [SMILES: COC(=O)c1ccc([C@H]2C[C@@H](Nc3nc4cc[nH]c4c(=O)n3C)CN(C)C2)cc1 ; CN1C[C@H](Nc2nc3cc[nH]c3c(=O)n2C)C[C@H](c2ccc(C(=O)O)cc2)C1]

To a stirred solution of ethyl 4-((3R,5R)-1-methyl-5-((3-methyl-4-oxo-4,5-dihydro-3H-pyrrolo[3,2-d]pyrimidin-2-yl)amino)piperidin-3-yl)benzoate (0.07 g, 0.17 mmol, from step 1) in ethanol (4 mL) and water (4 mL) was added lithium hydroxide (0.01 g, 0.68 mmol) at RT and the reaction mixture was stirred at RT for 1 h. The progress of the reaction was monitored by TLC. After completion of reaction, the reaction mixture was concentrated under reduced pressure. The precipitated solid was acidified with 1N aq. HCl, concentrated under vacuum and triturated with diethyl ether to afford 4-((3R,5R)-1-methyl-5-((3-methyl-4-oxo-4,5-dihydro-3H-pyrrolo[3,2-d]pyrimidin-2-yl)amino)piperidin-3-yl)benzoic acid (0.05 g, 76.92%) as pale yellow solid. LCMS: m/z 382.10 [M+H]⁺.

### Step 3: 3-(4-(9-(4-(3R,5R)-1-methyl-5-(3-methyl-4-oxo-4,5-dihydro-3H-pyrrolo[3,2-d]pyrimidin-2-yl)amino)piperidin-3-yl)benzoyl)-3,9-diazaspiro[5.5]undecan-3-yl)phenyl)piperidine-2,6-dione

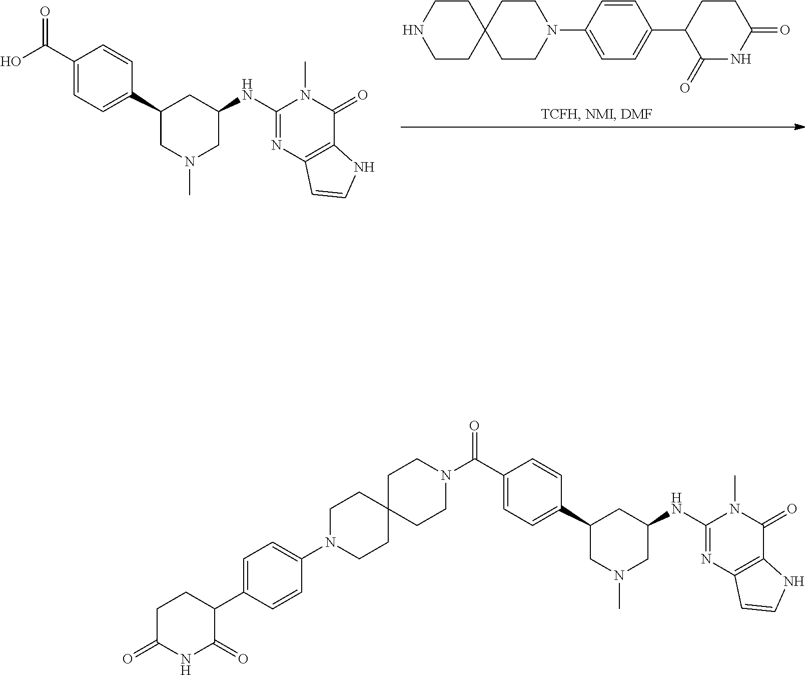 [SMILES: CN1C[C@H](Nc2nc3cc[nH]c3c(=O)n2C)C[C@H](c2ccc(C(=O)O)cc2)C1 ; O=C1CCC(c2ccc(N3CCC4(CCNCC4)CC3)cc2)C(=O)N1 ; CN1C[C@H](Nc2nc3cc[nH]c3c(=O)n2C)C[C@H](c2ccc(C(=O)N3CCC4(CC3)CCN(c3ccc(C5CCC(=O)NC5=O)cc3)CC4)cc2)C1]

To a stirred solution of 3-[4-(3,9-diazaspiro[5.5]undecan-3-yl)phenyl]piperidine-2,6-dione (0.05 g, 0.14 mmol, from general intermediate 22) in DMF (3 mL) were added 1-methylimidazole (0.05 mL, 0.60 mmol), 4-((3R,5R)-1-methyl-5-((3-methyl-4-oxo-4,5-dihydro-3H-pyrrolo[3,2-d]pyrimidin-2-yl)amino)piperidin-3-yl)benzoic acid (0.050 g, 0.13 mmol, from step 2), and TCFH (45 mg, 0.157 mmol), respectively. The reaction mixture was stirred at RT for 16 h. The progress of the reaction was monitored by TLC and LCMS. The reaction mixture was concentrated under vacuum to obtain crude product. The crude was purified by preparative HPLC [Column: BAKERBOND 1.8 um C18, (100 mm×2.1 mm); Mobile phase A: 0.05% formic acid in water, Mobile phase B: 0.05% formic acid in acetonitrile] to afford 3-(4-(9-(4-((3R,5R)-1-methyl-5-((3-methyl-4-oxo-4,5-dihydro-3H-pyrrolo[3,2-d]pyrimidin-2-yl)amino)piperidin-3-yl)benzoyl)-3,9-diazaspiro[5.5]undecan-3-yl)phenyl)piperidine-2,6-dione (0.007 g, 6.8%) as off white solid. ¹H NMR (400 MHz, DMSO-d₆) δ=11.42 (br s, 1H), 10.75 (s, 1H), 8.25 (s, 1H), 7.34 (s, 4H), 7.15 (t, J=2.8 Hz, 1H), 7.03 (d, J=8.5 Hz, 2H), 6.88 (d, J=8.8 Hz, 2H), 6.05 (t, J=2.3 Hz, 1H), 6.00 (d, J=7.8 Hz, 1H), 4.24-4.16 (m, 1H), 3.71 (dd, J=4.9, 10.9 Hz, 1H), 3.65-3.59 (m, 2H), 3.35 (s, 3H), 3.18-3.07 (m, 4H), 3.01-2.86 (m, 3H), 2.69-2.57 (m, 1H), 2.24 (s, 3H), 2.14-2.07 (m, 2H), 2.04-1.97 (m, 1H), 1.94-1.79 (m, 2H), 1.69-1.56 (m, 5H), 1.54-1.40 (m, 4H). Compound presented as formate salt. Three protons merged in solvent peak. LCMS: m/z 705.63 [M+H]⁺.

### Example 17: 3-(3-(4-(4-((1S,2S)-1-(dimethylamino)-2-((3-methyl-[1,2,4]triazolo[3,4-a]phthalazin-6-yl)amino)propyl)benzyl)piperazin-1-yl)phenyl)piperidine-2,6-dione (Compound 428)

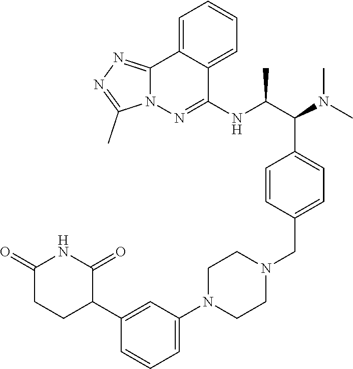 [SMILES: Cc1nnc2c3ccccc3c(N[C@@H](C)[C@H](c3ccc(CN4CCN(c5cccc(C6CCC(=O)NC6=O)c5)CC4)cc3)N(C)C)nn12]

### Step 1:(4-((1S,2S)-1-(dimethylamino)-2-((3-methyl-[1,2,4]triazolo[3,4-a]phthalazin-6-yl)amino)propyl)phenyl)methanol

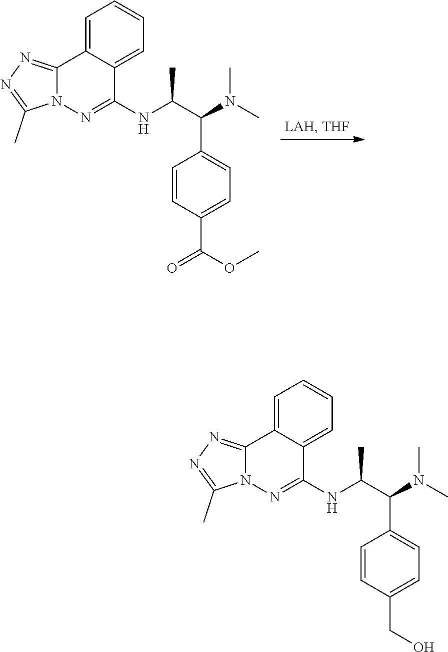 [SMILES: COC(=O)c1ccc([C@@H]([C@H](C)Nc2nn3c(C)nnc3c3ccccc23)N(C)C)cc1 ; Cc1nnc2c3ccccc3c(N[C@@H](C)[C@H](c3ccc(CO)cc3)N(C)C)nn12]

To a stirred solution of methyl 4-((1S,2S)-1-(dimethylamino)-2-((3-methyl-[1,2,4]triazolo[3,4-a]phthalazin-6-yl)amino)propyl)benzoate (0.08 g, 0.19 mmol, from step 4 in example 15) in tetrahydrofuran (3 mL) was added LAH (2 M) in THF (0.15 mL, 0.30 mmol) at 0° C. and the reaction mixture was stirred at 0° C. for 2 h. The progress of the reaction was monitored by TLC. After completion of reaction, the reaction mixture was quenched with saturated Na₂SO₄ solution and extracted with DCM. The combined organic layer was dried over anhydrous Na₂SO₄ and concentrated under reduced pressure to afford (4-((1S,2S)-1-(dimethylamino)-2-((3-methyl-[1,2,4]triazolo[3,4-a]phthalazin-6-yl)amino)propyl)phenyl)-methanol (0.06 g, 80.38%) as an off white solid, which was used as such for the next reaction. LC-MS: m/z 391.39 [M+H]⁺.

### Step 2: (1S,2S)-1-(4-(chloromethyl)phenyl)-N1,N1-dimethyl-N2-(3-methyl-[1,2,4]triazolo[3,4-a]phthalazin-6-yl)propane-1,2-diamine

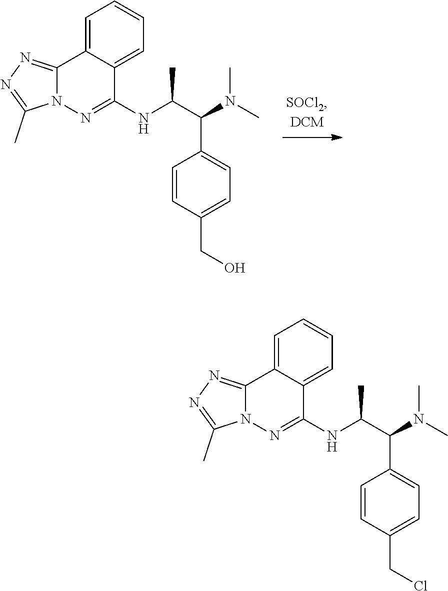 [SMILES: Cc1nnc2c3ccccc3c(N[C@@H](C)[C@H](c3ccc(CO)cc3)N(C)C)nn12 ; Cc1nnc2c3ccccc3c(N[C@@H](C)[C@H](c3ccc(CCl)cc3)N(C)C)nn12]

To a stirred solution of (4-((1S,2S)-1-(dimethylamino)-2-((3-methyl-[1,2,4]triazolo[3,4-a]phthalazin-6-yl)amino)propyl)phenyl)methanol (0.06 g, 0.15 mmol, from step 1) in dichloromethane (3 mL) was added thionyl chloride (0.20 mL, 3.00 mmol) at 0° C. Reaction mixture was stirred at RT for 1 h. Progress of reaction was monitored by TLC and LCMS. After completion of reaction, the reaction mixture was concentrated under reduced pressure to afford (1S,2S)-1-(4-(chloromethyl)phenyl)-N1,N1-dimethyl-N2-(3-methyl-[1,2,4]triazolo[3,4-a]phthalazin-6-yl) propane-1,2-diamine (0.05 g, 79.58%) as an off white solid, which was used as such for the next reaction. LC-MS: m/z 409.40 [M+H]⁺.

### Step 3: 3-(3-(4-(4-((1S,2S)-1-(dimethylamino)-2-((3-methyl-[1,2,4]triazolo[3,4-a]phthalazin-6-yl)amino)propyl)benzyl)piperazin-1-yl)phenyl)piperidine-2,6-dione

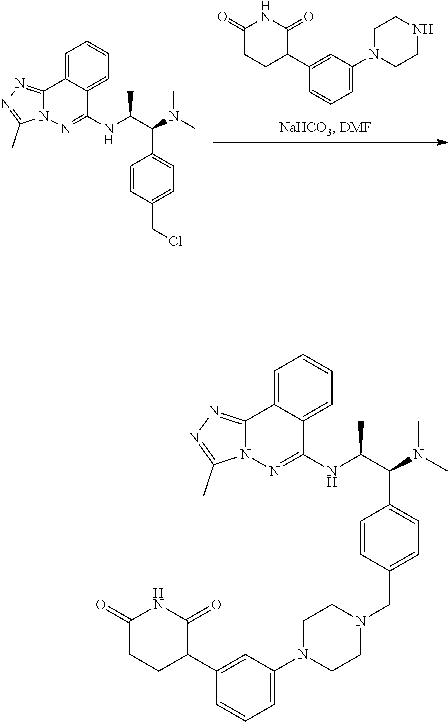 [SMILES: Cc1nnc2c3ccccc3c(N[C@@H](C)[C@H](c3ccc(CCl)cc3)N(C)C)nn12 ; O=C1CCC(c2cccc(N3CCNCC3)c2)C(=O)N1 ; Cc1nnc2c3ccccc3c(N[C@@H](C)[C@H](c3ccc(CN4CCN(c5cccc(C6CCC(=O)NC6=O)c5)CC4)cc3)N(C)C)nn12]

To a stirred solution of 3-(3-piperazin-1-ylphenyl)piperidine-2,6-dione (0.05 g, 0.18 mmol, from general intermediate 22) in DMF (3 mL) was added sodium bicarbonate (0.04 g, 0.54 mmol) at RT. The reaction mixture was stirred for 30 min. To this was added (1S,2S)-1-(4-(chloromethyl)phenyl)-N1,N1-dimethyl-N2-(3-methyl-[1,2,4]triazolo[3,4-a]phthalazin-6-yl) propane-1,2-diamine (0.05 g, 0.12 mmol, from step 2) at RT. Reaction mixture was stirred at 100° C. for 16 h. Progress of reaction was monitored by TLC and LCMS. After completion of reaction, the reaction mixture was filtered and washed with DCM. The filtrate was concentrated under reduced pressure to get crude. The crude was purified by preparative HPLC (Method E) to afford 3-(3-(4-(4-((1S,2S)-1-(dimethylamino)-2-((3-methyl-[1,2,4]triazolo[3,4-a]phthalazin-6-yl)amino)propyl)benzyl)piperazin-1-yl)phenyl)piperidine-2,6-dione (0.003 g) as an off white solid. ¹H NMR (400 MHz, DMSO-d₆) δ=10.77 (s, 1H), 8.41 (d, J=7.0 Hz, 1H), 8.29 (d, J=8.4 Hz, 1H), 7.94 (t, J=7.7 Hz, 1H), 7.86-7.81 (m, 1H), 7.34 (d, J=7.9 Hz, 2H), 7.21 (d, J=8.1 Hz, 2H), 7.15 (t, J=7.8 Hz, 1H), 7.09 (d, J=5.9 Hz, 1H), 6.86-6.76 (m, 2H), 6.64-6.58 (m, 1H), 4.78-4.62 (m, 1H), 3.78-3.69 (m, 2H), 3.55 (s, 2H), 3.18-3.08 (m, 4H), 2.64-2.61 (m, 4H), 2.57-2.53 (m, 4H), 2.46-2.45 (m, 1H), 2.20-2.09 (m, 7H), 2.06-1.99 (m, 1H), 1.11 (d, J=6.4 Hz, 3H), LC-MS: m/z 646.30 [M+H]⁺.

### Example 18: 3-(4-(9-(4-((1S,2S)-1-(dimethylamino)-2-((6-methyl-5-oxo-5,6-dihydropyrido[2,3-d]pyridazin-8-yl)amino)propyl)benzoyl)-3,9-diazaspiro[5.5]undecan-3-yl)phenyl)piperidine-2,6-dione (Compound 429)

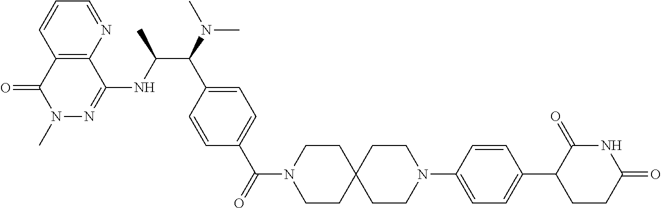 [SMILES: C[C@H](Nc1nn(C)c(=O)c2cccnc12)[C@H](c1ccc(C(=O)N2CCC3(CC2)CCN(c2ccc(C4CCC(=O)NC4=O)cc2)CC3)cc1)N(C)C]

### Step 1: 6-methyl-6,7-dihydropyrido[2,3-d]pyridazine-5,8-dione

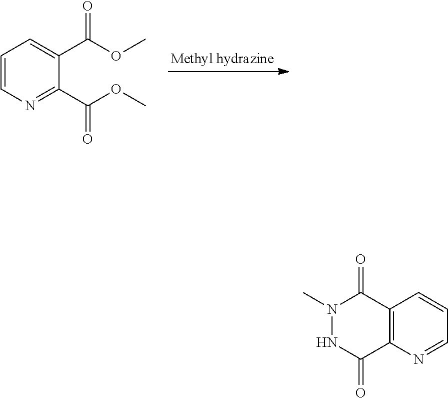

To a stirred solution of dimethyl pyridine-2,3-dicarboxylate (10.00 g, 51.23 mmol) in ethanol (80 mL) was added methylhydrazine (85% aq, 15 mL) and the reaction mixture was heated at 100° C. for 16 h. Progress of reaction was monitored by TLC. After completion of the reaction, the reaction mixture was cooled to RT, concentrated under reduced pressure, and extracted with DCM. The combined organic layer was dried over anhydrous Na₂SO₄, filtered, and concentrated under reduced pressure to obtain crude product. The crude was purified by combi flash using 20% MeOH/DCM to afford 6-methyl-6,7-dihydropyrido[2,3-d]pyridazine-5,8-dione (3.20 g, 35%) as an off white solid. ¹H NMR (400 MHz, DMSO-d₆) δ=11.07 (brs, 1H), 9.11 (d, J=2.9 Hz, 1H), 8.58 (d, J=6.8 Hz, 1H), 7.95-7.78 (m, 1H), 3.58 (s, 3H).

### Step 2: 8-chloro-6-methylpyrido[2,3-d]pyridazin-5 (6H)-one

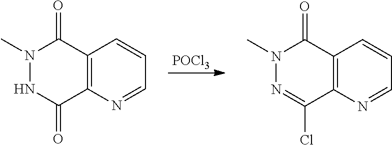

To a stirred solution of 6-methyl-6,7-dihydropyrido[2,3-d]pyridazine-5,8-dione (1.50 g, 8.50 mmol, from step 1) was added phosphorus (V) oxychloride (20 mL) at RT. The reaction mixture was stirred at 100° C. for 16 h. The progress of the reaction was monitored by TLC. After completion of reaction, the reaction mixture was quenched with ice water and basified with NaHCO₃ and extracted with ethyl acetate. The combined organic layers were dried over anhydrous Na₂SO₄ and concentrated under reduced pressure. The crude compound was purified by silica column chromatography using 50% ethyl acetate/hexane as eluent to afford 8-chloro-6-methylpyrido[2,3-d]pyridazin-5 (6H)-one (1.02 g, 62%) as an off white solid. LC-MS: m/z 196.06 [M+H]⁺.

### Step 3: Methyl 4-((1S,2S)-1-(dimethylamino)-2-((6-methyl-5-oxo-5,6-dihydropyrido[2,3-d]pyridazin-8-yl)amino)propyl)benzoate

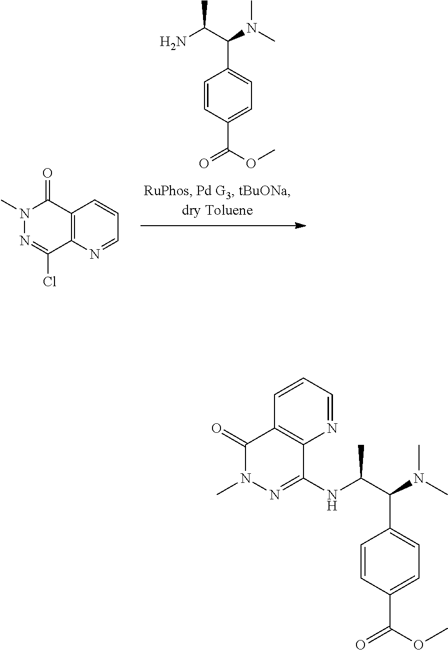 [SMILES: COC(=O)c1ccc([C@@H]([C@H](C)N)N(C)C)cc1 ; Cn1nc(Cl)c2ncccc2c1=O ; COC(=O)c1ccc([C@@H]([C@H](C)Nc2nn(C)c(=O)c3cccnc23)N(C)C)cc1]

To a stirred solution of methyl 4-[(1S,2S)-2-amino-1-(dimethylamino)propyl]benzoate (0.20 g, 0.84 mmol) in toluene (5 mL) was added 8-chloro-6-methylpyrido[2,3-d]pyridazin-5 (6H)-one (0.24 g, 1.26 mmol, from step 2) followed by sodium tort-butoxide (0.16 g, 1.69 mmol) at RT and reaction mixture was degassed with argon for 5 min. To this (2-dicyclohexylphosphino-2′,6′-diisopropoxy-1,1′-biphenyl)[2-(2′-amino-1,1′-biphenyl)]palladium(II) methanesulfonate (0.08 g, 0.09 mmol) was added at RT. The reaction mixture was stirred in a microwave at 110° C. for 2 h. The progress of the reaction was monitored by LCMS. Reaction mixture was filtered through celite and washed with ethyl acetate. Filtrate was concentrated under reduced pressure, and the crude was purified by silica gel column chromatography using 80% ethyl acetate in hexane as eluent to afford methyl 4-((1S,2S)-1-(dimethylamino)-2-((6-methyl-5-oxo-5,6-dihydropyrido[2,3-d]pyridazin-8-yl)amino)propyl)benzoate (0.03 g, 8.96%) as brown solid. LC-MS: m/z 396.25 [M+H]⁺.

### Step 4: 4-((1S,2S)-1-(dimethylamino)-2-((6-methyl-5-oxo-5,6-dihydropyrido[2,3-d]pyridazin-8-yl)amino)propyl)benzoic acid

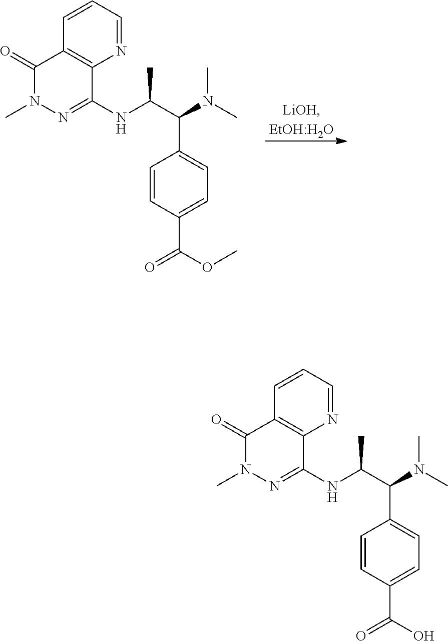 [SMILES: COC(=O)c1ccc([C@@H]([C@H](C)Nc2nn(C)c(=O)c3cccnc23)N(C)C)cc1 ; C[C@H](Nc1nn(C)c(=O)c2cccnc12)[C@H](c1ccc(C(=O)O)cc1)N(C)C]

To a stirred solution of methyl 4-((1S,2S)-1-(dimethylamino)-2-((6-methyl-5-oxo-5,6-dihydropyrido[2,3-d]pyridazin-8-yl)amino)propyl)benzoate (0.05 g, 0.12 mmol, from step 3) in ethanol (3 mL) were added lithium hydroxide (0.008 g, 0.19 mmol) and water (0.2 mL) at RT. The reaction mixture was stirred at 80° C. for 2 h. The progress of the reaction was monitored by TLC. After completion of reaction, the reaction mixture was concentrated under reduced pressure, 2N HCl was added, Solid precipitated was formed and filtered and was washed with diethyl ether, then dried under reduced pressure to afford 4-((1S,2S)-1-(dimethylamino)-2-((6-methyl-5-oxo-5,6-dihydropyrido[2,3-d]pyridazin-8-yl)amino)propyl)-benzoic acid as an off white solid. LC-MS: m/z 382.12 [M+H]⁺.

### Step 5: 3-(4-(9-(4-((1S,2S)-1-(dimethylamino)-2-((6-methyl-5-oxo-5,6-dihydropyrido[2,3-d]pyridazin-8-yl)amino)propyl)benzoyl)-3,9-diazaspiro[5.5]undecan-3-yl)phenyl)-piperidine-2,6-dione

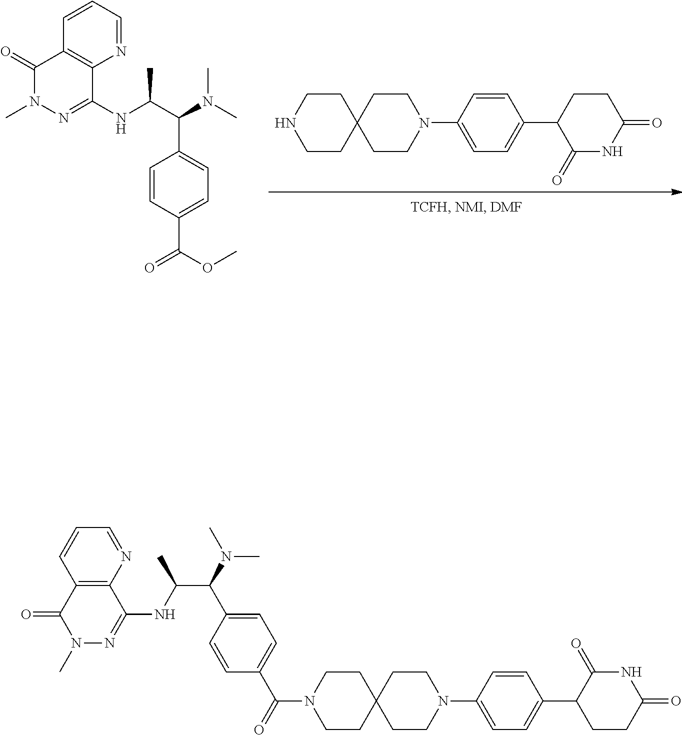 [SMILES: COC(=O)c1ccc([C@@H]([C@H](C)Nc2nn(C)c(=O)c3cccnc23)N(C)C)cc1 ; O=C1CCC(c2ccc(N3CCC4(CCNCC4)CC3)cc2)C(=O)N1 ; C[C@H](Nc1nn(C)c(=O)c2cccnc12)[C@H](c1ccc(C(=O)N2CCC3(CC2)CCN(c2ccc(C4CCC(=O)NC4=O)cc2)CC3)cc1)N(C)C]

To the stirred solution of 3-[4-(3,9-diazaspiro[5.5]undecan-3-yl)phenyl]piperidine-2,6-dione (0.05 g, 0.12 mmol, from general intermediate 22) in DMF (1 mL) was added 1-methylimidazole (0.04 mL, 0.52 mmol) followed by 4-((1S,2S)-1-(dimethylamino)-2-((6-methyl-5-oxo-5,6-dihydropyrido[2,3-d]pyridazin-8-yl)amino)propyl)benzoic acid (0.04 g, 0.10 mmol, from step 4) at RT. To this N,N,N′,N′-tetramethylchloroformamidinium hexafluorophosphate (0.04 g, 0.15 mmol) was added at RT and reaction mixture was stirred at RT for 16 h. After completion of the reaction, the reaction mixture was quenched with ice-water, solid formed was filtered, dried under reduced pressure to get crude product. The crude was purified by preparative HPLC (Method E) to afford 3-(4-(9-(4-((1S,2S)-1-(dimethylamino)-2-((6-methyl-5-oxo-5,6-dihydropyrido[2,3-d]pyridazin-8-yl)amino)propyl)benzoyl)-3,9-diazaspiro[5.5]undecan-3-yl)phenyl)piperidine-2,6-dione as an off white solid. ¹H NMR (400 MHz, DMSO-d₆) δ=10.75 (s, 1H), 9.09 (dd, J=1.6, 4.5 Hz, 1H), 8.61 (dd, J=1.6, 8.0 Hz, 1H), 7.93-7.86 (m, 1H), 7.46-7.38 (m, 2H), 7.37-7.30 (m, 2H), 7.03 (br d, J=8.8 Hz, 2H), 6.93-6.83 (m, 3H), 4.29-4.22 (m, 1H), 3.74-3.68 (m, 2H), 3.66-3.60 (m, 4H), 3.42-3.35 (m, 4H), 3.18-3.10 (m, 4H), 2.64-2.56 (m, 2H), 2.10-2.06 (m, 6H), 2.03-1.96 (m, 1H), 1.66-1.59 (m, 4H), 1.55-1.46 (m, 4H), 1.09 (d, J=6.0 Hz, 3H). LC-MS: m/z 705.63 [M+H]⁺.

### Example 19: 3-(4-(9-(4-((1S,2S)-1-(dimethylamino)-2-((7-methyl-8-oxo-7,8-dihydropyrido[2,3-d]pyridazin-5-yl)amino)propyl)benzoyl)-3,9-diazaspiro[5.5]undecan-3-yl)phenyl)-piperidine-2,6-dione (Compound 430)

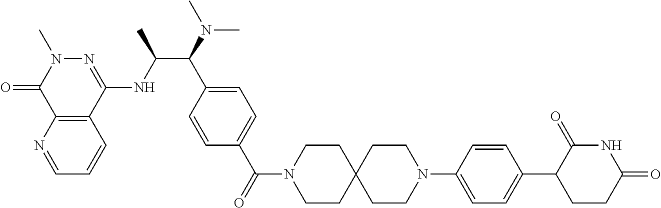 [SMILES: C[C@H](Nc1nn(C)c(=O)c2ncccc12)[C@H](c1ccc(C(=O)N2CCC3(CC2)CCN(c2ccc(C4CCC(=O)NC4=O)cc2)CC3)cc1)N(C)C]

### Step 1: 7-methyl-6,7-dihydropyrido[2,3-d]pyridazine-5,8-dione

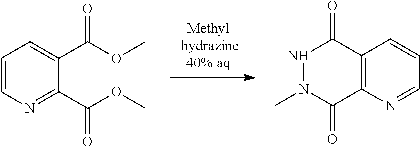

To a stirred solution of dimethyl pyridine-2,3-dicarboxylate (10.00 g, 51.23 mmol) in ethanol (80 mL) was added methylhydrazine (15 mL) and the reaction mixture was heated at 100° C. for 16 h. Reaction progress was monitored by TLC. After completion of the reaction, the reaction mixture was cooled to room temperature and ethanol was distilled off under reduced pressure and extracted with 20% MeOH in DCM. The combined organic layer was dried over anhydrous sodium sulphate, filtered, and concentrated under reduced pressure to afford crude compound. The crude compound was purified by silica gel column chromatography eluting with 20% MeOH in DCM to afford 7-methyl-6,7-dihydropyrido[2,3-d]pyridazine-5,8-dione (3.2 g, 35%) as off white solid. ¹H NMR (400 MHz, DMSO-d₆) δ=11.60-10.95 (m, 1H), 9.11 (d, J=2.9 Hz, 1H), 8.59-8.57 (m, 1H), 7.89-7.85 (m, 1H), 3.57 (s, 3H).

### Step 2: 5-chloro-7-methylpyrido[2,3-d]pyridazin-8(7H)-one

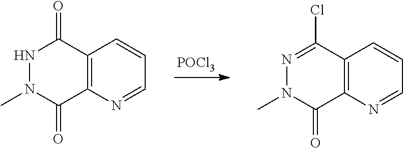

A solution of 7-methyl-6,7-dihydropyrido[2,3-d]pyridazine-5,8-dione (2.5 g, 14 mmol, from step 1) in phosphorus (V) oxychloride (50 mL) was stirred at 100° C. for 16 h. The progress of the reaction was monitored by TLC. After completion of reaction, the reaction mixture was added to ice water, basified by aq. NaHCO₃ solution, and extracted with ethyl acetate. The organic layer was dried over anhydrous sodium sulphate, filtered, and concentrated reduced pressure to obtain crude compound. The crude compound was purified by silica gel column chromatography using 60-70% EtOAc in heptane as eluent to afford 5-chloro-7-methylpyrido[2,3-d]pyridazin-8 (7H)-one (0.16 g, 6%) as brown solid. LC-MS: m/z 196.00 [M+H]⁺.

### Step 3: Methyl 4-((1S,2S)-1-(dimethylamino)-2-((7-methyl-8-oxo-7,8-dihydropyrido[2,3-d]pyridazin-5-yl)amino)propyl)benzoate

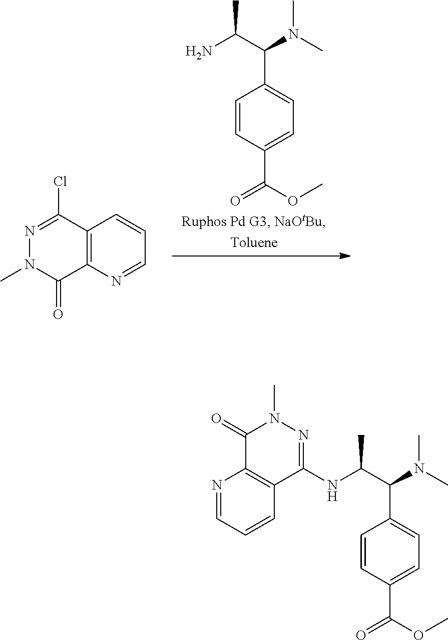 [SMILES: Cn1nc(Cl)c2cccnc2c1=O ; COC(=O)c1ccc([C@@H]([C@H](C)N)N(C)C)cc1 ; COC(=O)c1ccc([C@@H]([C@H](C)Nc2nn(C)c(=O)c3ncccc23)N(C)C)cc1]

To a stirred solution of methyl 4-[(1S,2S)-2-amino-1-(dimethylamino)propyl]benzoate (0.15 g, 0.63 mmol, from step 3 of example 15) and 5-chloro-7-methylpyrido[2,3-d]pyridazin-8(7H)-one (0.15-g, 0.76 mmol, from step 2) in toluene (4 mL) was added sodium tert-butoxide (0.12 g, 1.26 mmol) and then the reaction mixture was degassed using argon for 5 min. Then (2-Dicyclohexylphosphino-2′,6′-diisopropoxy-1,1′-biphenyl)[2-(2′-amino-1,1′-biphenyl)]palladium(II) methanesulfonate (0.05 g, 0.06 mmol) was added and the reaction mixture was stirred in a microwave at 110° C. for 2 h. The progress of the reaction was monitored by TLC. After completion of reaction, the reaction mixture was filtered through a celite pad and washed with ethyl acetate. The filtrate was concentrated under reduced pressure to afford crude compound. The crude was purified by silica gel column chromatography eluting the product with 70-80% ethyl acetate in heptane to afford methyl 4-((1S,2S)-1-(dimethylamino)-2-((7-methyl-8-oxo-7,8-dihydropyrido[2,3-d]pyridazin-5-yl)amino)propyl)benzoate (0.06 g, 21%) as brown solid. LC-MS: m/z 396.17 [M+H]⁺.

### Step 4: 4-((1S,2S)-1-(dimethylamino)-2-((7-methyl-8-oxo-7,8-dihydropyrido[2,3-d]pyridazin-5-yl)amino)propyl)benzoic acid

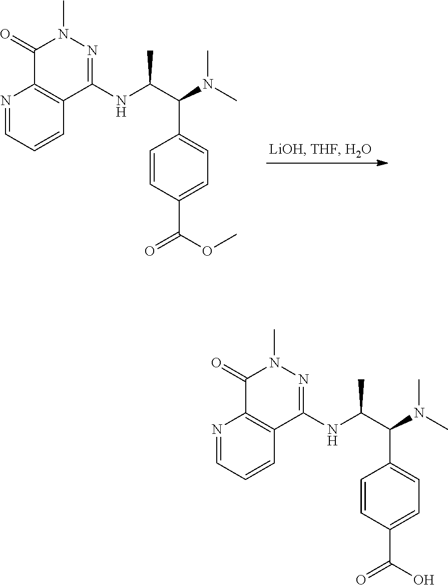 [SMILES: COC(=O)c1ccc([C@@H]([C@H](C)Nc2nn(C)c(=O)c3ncccc23)N(C)C)cc1 ; C[C@H](Nc1nn(C)c(=O)c2ncccc12)[C@H](c1ccc(C(=O)O)cc1)N(C)C]

To a stirred solution of methyl 4-((1S,2S)-1-(dimethylamino)-2-((7-methyl-8-oxo-7,8-dihydropyrido[2,3-d]pyridazin-5-yl)amino)propyl)benzoate (0.05 g, 0.12 mmol, from step 3) in ethanol (3 mL) and water (0.5 mL) was added lithium hydroxide (0.001 g, 0.19 mmol at RT and the reaction mixture was stirred at 80° C. for 2 h. The progress of the reaction was monitored by TLC. After completion of reaction, the reaction mixture was concentrated and acidified using 2 N HCl. Solids precipitated out were filtered off, washed with diethyl ether and dried under vacuum to afford 4-((1S,2S)-1-(dimethylamino)-2-((7-methyl-8-oxo-7,8-dihydropyrido[2,3-d]pyridazin-5-yl)amino)propyl)benzoic acid (0.04 g, 78%) as off white solid. LC-MS: m/z 382.16 [M+H]⁺.

### Step 5: 3-(4-(9-(4-((1S,2S)-1-(dimethylamino)-2-((7-methyl-8-oxo-7,8-dihydropyrido[2,3-d]pyridazin-5-yl)amino)propyl)benzoyl)-3,9-diazaspiro[5.5]undecan-3-yl)phenyl)-piperidine-2,6-dione

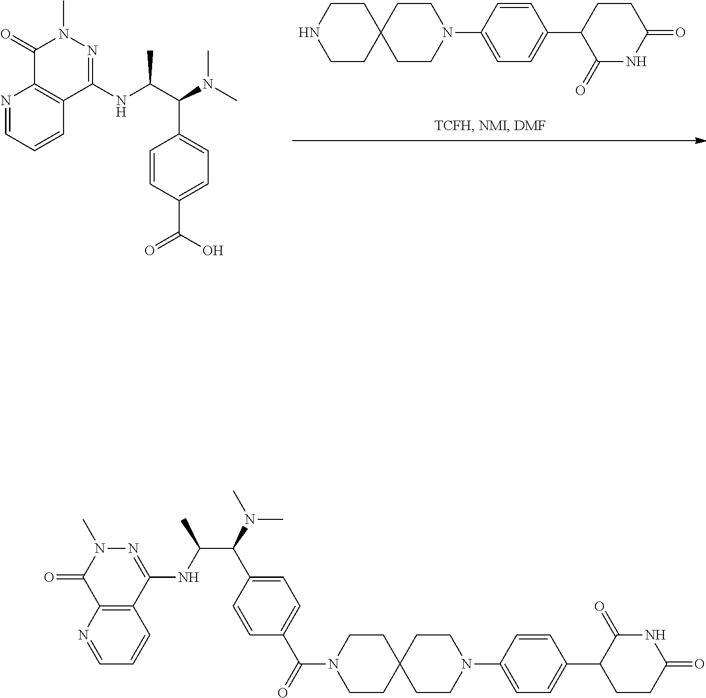 [SMILES: C[C@H](Nc1nn(C)c(=O)c2ncccc12)[C@H](c1ccc(C(=O)O)cc1)N(C)C ; O=C1CCC(c2ccc(N3CCC4(CCNCC4)CC3)cc2)C(=O)N1 ; C[C@H](Nc1nn(C)c(=O)c2ncccc12)[C@H](c1ccc(C(=O)N2CCC3(CC2)CCN(c2ccc(C4CCC(=O)NC4=O)cc2)CC3)cc1)N(C)C]

To the stirred solution of 3-[4-(3,9-diazaspiro[5.5]undecan-3-yl)phenyl]piperidine-2,6-dione (0.04 g, 0.12 mmol, from general intermediate 22) in DMF (I mL), were added 1-methylimidazole (0.05 mL, 0.6 mmol) and 4-((1S,2S)-1-(dimethylamino)-2-((7-methyl-8-oxo-7,8-dihydropyrido[2,3-d]pyridazin-5-yl)amino)propyl)benzoic acid (0.04 g, 0.10 mmol, from step 4) and stirred for 2 min. Then TCFH (0.04 g, 0.15 mmol) was added, and reaction was stirred at RT for 16 h. The progress of the reaction was monitored by LCMS. Reaction mixture was mixed with ice-water, solids formed were filtered off, dried under vacuum to afford crude compound. The crude compound was purified by preparative HPLC [Column: X-BRIDGE C18 (250*30 mm) 5μ; Mobile phase A: 10 mM ammonium bicarbonate in water, Mobile phase B: 100% acetonitrile] to afford 3-(4-(9-(4-((1S,2S)-1-(dimethylamino)-2-((7-methyl-8-oxo-7,8-dihydropyrido[2,3-d]pyridazin-5-yl)amino)propyl)benzoyl)-3,9-diazaspiro[5.5]undecan-3-yl)phenyl)piperidine-2,6-dione (0.008 g, 11%) as off white solid. ¹H NMR (400 MHz, DMSO-d₆) δ=10.75 (s, 1H), 9.09 (dd, J=4.6, 1.7 Hz, 1H), 8.61 (dd, J=8.1, 1.8 Hz, 1H), 7.89 (dd, J=8.0, 4.4 Hz, 1H), 7.40 (d, J=8.0 Hz, 2H), 7.34 (d, J=8.8 Hz, 2H), 7.03 (d, J=8.8 Hz, 2H), 6.90-6.86 (m, 3H), 4.29-4.22 (m, 1H), 3.74-3.69 (m, 2H), 3.67-3.59 (m, 4H), 3.14 (brs, 4H), 2.64-2.58 (m, 1H), 2.48-2.41 (m, 2H), 2.17-1.98 (m, 8H), 1.65-1.59 (m, 4H), 1.50 (d, J=2.8 Hz, 4H), 1.09 (d, J=6.0 Hz, 3H). Note: Two protons merged with solvent peak. LC-MS: m/z 705.30 [M+H]⁺.

### Example 20: 6-(((1S,2S)-1-(dimethylamino)-1-(4-(9-(4-(2,6-dioxopiperidin-3~yl)phenyl)-3,9-diazaspiro[5.5]undecane-3-carbonyl)phenyl) propan-2-yl)amino)-2,4-dimethyl-1,2,4-triazine-3,5(2H,4H)-dione (Compound 431)

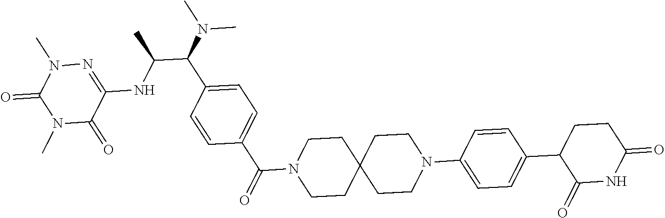 [SMILES: C[C@H](Nc1nn(C)c(=O)n(C)c1=O)[C@H](c1ccc(C(=O)N2CCC3(CC2)CCN(c2ccc(C4CCC(=O)NC4=O)cc2)CC3)cc1)N(C)C]

### Step 1: 6-Bromo-2,4-dimethyl-1,2,4-triazine-3,5(2H,4H)-dione

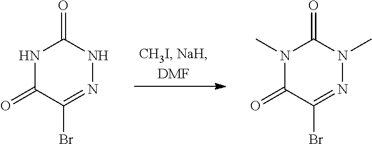

To a stirred solution of 6-bromo-2H-1,2,4-triazine-3,5-dione (2 g, 10.41 mmol) in N,N-dimethylformamide (30 mL, 388 mmol) was added sodium hydride (60% in oil, 1.04 g, 26.04 mmol) portion-wise at 0° C. and reaction mixture was stirred at 0° C. for 15 min. To this iodomethane (1.63 mL, 26.2 mmol) was added dropwise and reaction was stirred at 0° C. to RT for 3 h. The progress of the reaction was monitored by TLC. Reaction was quenched with methanol and concentrated under reduced pressure to afford crude compound. The crude compound was purified by silica gel column chromatography where the product was eluted with 30% EtOAc in heptane to afford 6-bromo-2,4-dimethyl-1,2,4-triazine-3,5(2H,4H)-dione (1.40 g, 61%) as an off white solid. LC-MS: m/z 221.88 [M+H]⁺.

### Step 2: Methyl 4-((1S,2S)-2-((2,4-dimethyl-3,5-dioxo-2,3,4,5-tetrahydro-1,2,4-triazin-6-yl)amino)-1-(dimethylamino)propyl)benzoate

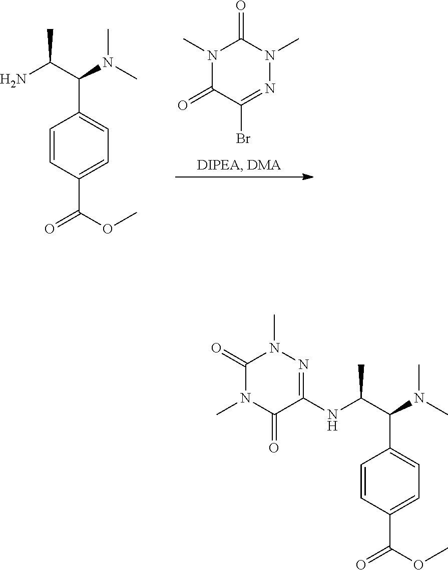

To a stirred solution of methyl 4-((1S,2S)-2-amino-1-(dimethylamino)propyl)benzoate (0.20 g, 0.84 mmol) and 6-bromo-2,4-dimethyl-1,2,4-triazine-3,5-dione (0.50 g, 2.27 mmol, from step 1) in N,N-dimethylacetamide (0.3 mL, 3 mmol), was added N,N-diisopropylethylamine (0.89 g, 6.8 mmol) and the reaction mixture was degassed using argon for 5 min. The reaction mixture was stirred at 180° C. for 2 h in a microwave. The progress of the reaction was monitored by LCMS. After completion of reaction, the reaction mixture was extracted with ethyl acetate. The organic layer was dried over anhydrous sodium sulphate, filtered, and concentrated under reduced pressure to obtain crude compound. The crude was purified by silica gel column chromatography where the product was eluted with 70% EtOAc in heptane to afford methyl 4-((1S,2S)-2-((2,4-dimethyl-3,5-dioxo-2,3,4,5-tetrahydro-1,2,4-triazin-6-yl)amino)-1-(dimethylamino)propyl)benzoate (0.20 g, 23%) as a yellow semi solid. LC-MS: m/z 376.20 [M+H]⁺.

### Step 3: 4-((1S,2S)-2-((2,4-dimethyl-3,5-dioxo-2,3,4,5-tetrahydro-1,2,4-triazin-6-yl)amino)-1-(dimethylamino)propyl)benzoic acid

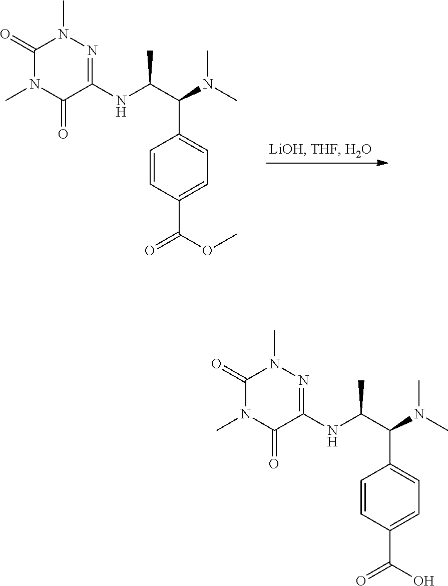 [SMILES: COC(=O)c1ccc([C@@H]([C@H](C)Nc2nn(C)c(=O)n(C)c2=O)N(C)C)cc1 ; C[C@H](Nc1nn(C)c(=O)n(C)c1=O)[C@H](c1ccc(C(=O)O)cc1)N(C)C]

To a stirred solution of methyl 4-((1S,2S)-2-((2,4-dimethyl-3,5-dioxo-2,3,4,5-tetrahydro-1,2,4-triazin-6-yl)amino)-1-(dimethylamino)propyl)benzoate (0.20 g, 0.53 mmol, from step 2) in THF (1 mL) and methanol (1 mL) and water (1 mL), was added lithium hydroxide monohydrate (0.03 g, 1.25 mmol) and the reaction mixture was stirred at 80° C. for 2 h. The progress of the reaction was monitored by TLC. After completion of the reaction, the reaction mixture was concentrated under reduced pressure and then acidified using 1N HCl. The solids were dried under vacuum to afford 4-((1S,2S)-2-((2,4-dimethyl-3,5-dioxo-2,3,4,5-tetrahydro-1,2,4-triazin-6-yl)amino)-1-(dimethylamino)propyl)benzoic acid (0.18 g, 96%) as a brown solid. LC-MS: m/z 362.37 [M+H]⁺.

### Step 4: 6-(((1S,2S)-1-(dimethylamino)-1-(4-(9-(4-(2,6-dioxopiperidin-3-yl)phenyl)-3,9-diazaspiro[5.5]undecane-3-carbonyl)phenyl) propan-2-yl)amino)-2,4-dimethyl-1,2,4-triazine-3,5(2H,4H)-dione

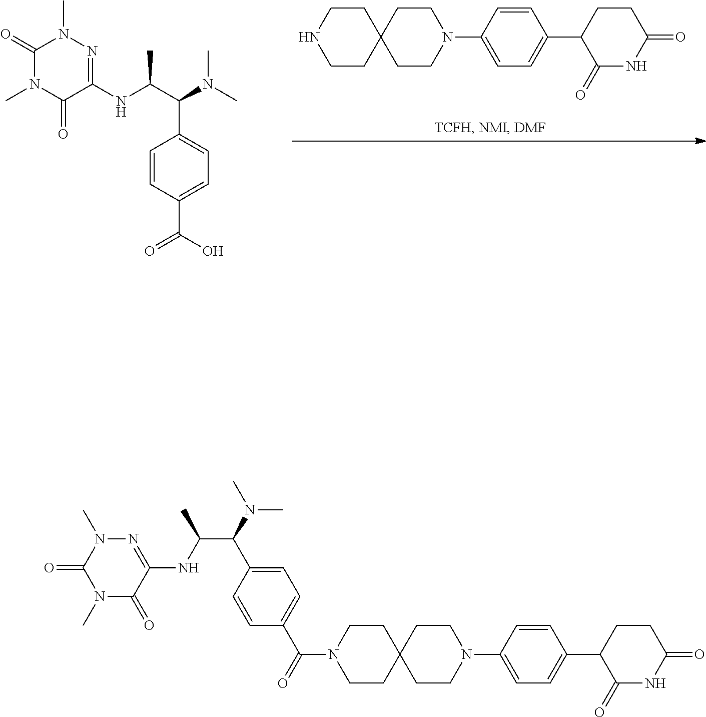 [SMILES: C[C@H](Nc1nn(C)c(=O)n(C)c1=O)[C@H](c1ccc(C(=O)O)cc1)N(C)C ; O=C1CCC(c2ccc(N3CCC4(CCNCC4)CC3)cc2)C(=O)N1 ; C[C@H](Nc1nn(C)c(=O)n(C)c1=O)[C@H](c1ccc(C(=O)N2CCC3(CC2)CCN(c2ccc(C4CCC(=O)NC4=O)cc2)CC3)cc1)N(C)C]

To a stirred solution of 4-((1S,2S)-2-((2,4-dimethyl-3,5-dioxo-2,3,4,5-tetrahydro-1,2,4-triazin-6-yl)amino)-1-(dimethylamino)propyl)benzoic acid (0.15 g, 0.41 mmol, from step 3) in DMF (5 mL) was added 1-methylimidazole (0.17 g, 2.07 mmol) and the reaction mixture was stirred at RT for 10 min. Then 3-[4-(3,9-diazaspiro[5.5]undecan-3-yl)phenyl]piperidine-2,6-dione (0.16 g, 0.47 mmol, from general intermediate 22) was added to the reaction mixture followed by TCFH (0.17 g, 0.61 mmol) and the reaction mixture was stirred at RT for 16 h. The progress of the reaction was monitored by TLC and LCMS. After completion of reaction, the reaction mixture was diluted with ice-cold water and extracted with 10% MeOH in DCM. The organic layer was dried over anhydrous sodium sulphate, filtered, and concentrated under reduced pressure, to afford crude compound. The crude was purified by preparative HPLC (Column: X-BRIDGE C18 (250*30 mm) 5μ, Mobile Phase A: 10 mM ABC in H₂O, Mobile Phase B: 100% ACN) to afford 6-(((1S,2S)-1-(dimethylamino)-1-(4-(9-(4-(2,6-dioxopiperidin-3-yl)phenyl)-3,9-diazaspiro[5.5]undecane-3-carbonyl)phenyl) propan-2-yl)amino)-2,4-dimethyl-1,2,4-triazine-3,5(2H,4H)-dione (0.01 g, 6%) as a white solid. ¹H NMR (400 MHz, DMSO-d₆) δ=10.75 (s, 1H), 7.39 (d, J=8.0 Hz, 2H), 7.29 (d, J=8.0 Hz, 2H), 7.03 (d, J=8.8 Hz, 2H), 6.89 (d, J=8.8 Hz, 2H), 6.30 (d, J=4.4 Hz, 1H), 4.07-4.01 (m, 1H), 3.71 (dd, J=4.9, 10.8 Hz, 1H), 3.66-3.60 (m, 3H), 3.41 (s, 3H), 3.30-3.28 (m, 2H), 3.21 (s, 3H), 3.18-3.13 (br s, 4H), 2.62-2.58 (m, 1H), 2.45-2.41 (m, 1H), 2.14-2.08 (m, 1H), 2.04 (s, 6H), 2.01-1.98 (m, 1H), 1.62 (br s, 4H), 1.55-1.43 (m, 4H), 0.98 (d, J=6.1 Hz, 3H). LC-MS: m/z 685.62 [M+H]⁺.

### Example 21: 3-(4-(9-(4-((1S,2S)-1-(dimethylamino)-2-((3-methyl-4-oxo-3,4-dihydro-phthalazin-1-yl)amino)propyl)benzoyl)-3,9-diazaspiro[5.5]undecan-3-yl)phenyl)piperid-ine-2,6-dione (Compound 432)

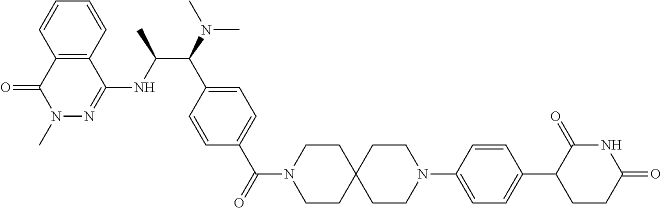 [SMILES: C[C@H](Nc1nn(C)c(=O)c2ccccc12)[C@H](c1ccc(C(=O)N2CCC3(CC2)CCN(c2ccc(C4CCC(=O)NC4=O)cc2)CC3)cc1)N(C)C]

### Step 1: 2-Methyl-2,3-dihydrophthalazine-1,4-dione

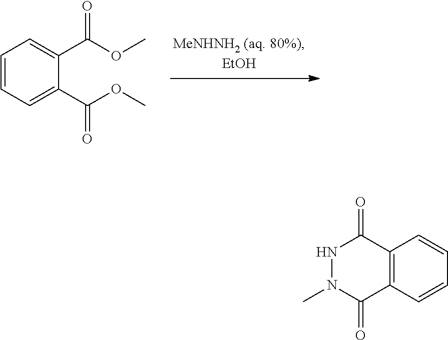

To a stirred solution of dimethyl phthalate (8 g, 41.19 mmol) in ethanol (20 mL) was added methylhydrazine (12 mL, 223 mmol) (80% aqueous solution) at RT and the reaction mixture was stirred at 80° C. for 16 h. The progress of the reaction was monitored by TLC and LCMS. After completion of the reaction, the mixture was concentrated under reduced pressure and triturated with heptane to afford 2-methyl-2,3-dihydrophthalazine-1,4-dione (4 g, 55%) as white solid. LCMS: m/z 177.0 [M+H]⁺.

### Step 2: 4-Chloro-2-methylphthalazin-1(2H)-one

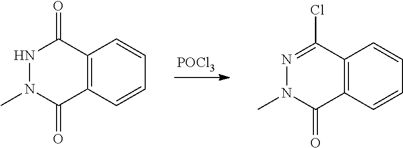

The solution of 2-methyl-2,3-dihydrophthalazine-1,4-dione (4.00 g, 22.70 mmol, from step 1) in phosphorus (V) oxychloride (50 mL, 531.1 mmol) was stirred at 100° C. for 48 h. The progress of the reaction was monitored by LCMS. After completion of the reaction, reaction mixture was concentrated under reduced pressure, then quenched with saturated Sodium bicarbonate solution and extracted with ethyl acetate. The combined organic layer was dried over anhydrous sodium sulphate, filtered, and concentrated under reduced pressure to get crude compound which was purified by silica gel column chromatography eluting with 4% ethyl acetate in heptane to afford 4-chloro-2-methyl-phthalazin-1-one (1.20 g, 27%) as an orange solid. LCMS: m/z 195.19 [M+H]⁺.

### Step 3: Methyl 4-((1S,2S)-1-(dimethylamino)-2-((3-methyl-4-oxo-3,4-dihydrophthalazin-1-yl)amino)propyl)benzoate

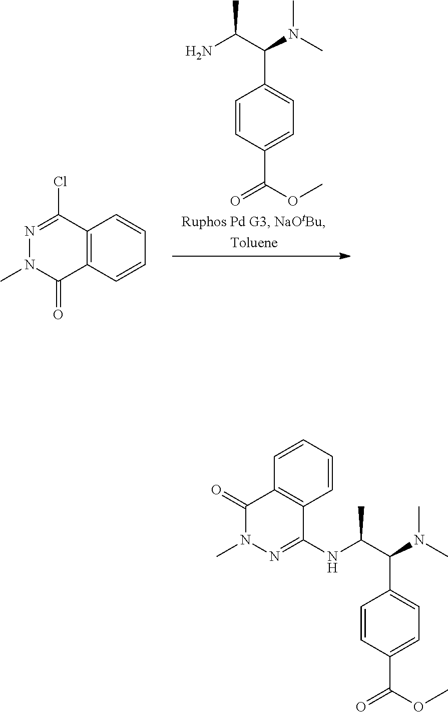 [SMILES: Cn1nc(Cl)c2ccccc2c1=O ; COC(=O)c1ccc([C@@H]([C@H](C)N)N(C)C)cc1 ; COC(=O)c1ccc([C@@H]([C@H](C)Nc2nn(C)c(=O)c3ccccc23)N(C)C)cc1]

To a stirred solution of 4-chloro-2-methyl-phthalazin-1-one (0.25 g, 1.28 mmol, from step 2) and methyl 4-[(1S,2S)-2-amino-1-(dimethylamino)propyl]benzoate (0.20 g, 0.84 mmol, from step 3 of example 15) in toluene (5 mL) was added sodium tert-butoxide (0.16 g, 1.68 mmol) and the reaction mixture was purged with argon for 10 min. To the resulting reaction mixture was added (2-dicyclohexylphosphino-2′,6′-diisopropoxy-1,1′-biphenyl) [2-(2′-amino-1,1′-biphenyl)]-palladium(II) methanesulfonate (0.07 g, 0.08 mmol) and the reaction mixture was again purged with argon for 10 min. The reaction mixture was stirred at 110° C. for 2 h in a microwave. The progress of the reaction was monitored by TLC and LCMS. After completion of reaction, the reaction mixture was extracted with ethyl acetate. The combined organic layer was dried over anhydrous sodium sulphate, filtered, and concentrated to obtain crude. The crude compound was purified by silica gel column chromatography where the product was eluted with 3% MeOH in DCM to afford methyl 4-((1S,2S)-1-(dimethylamino)-2-((3-methyl-4-oxo-3,4-dihydrophthalazin-1-yl)amino)propyl)benzoate (0.14 g, 42%) as a yellow solid. LCMS: m/z 395.40 [M+H]⁺.

### Step 4: 4-((1S,2S)-1-(dimethylamino)-2-((3-methyl-4-oxo-3,4-dihydrophthalazin-1-yl)-amino)propyl)benzoic acid

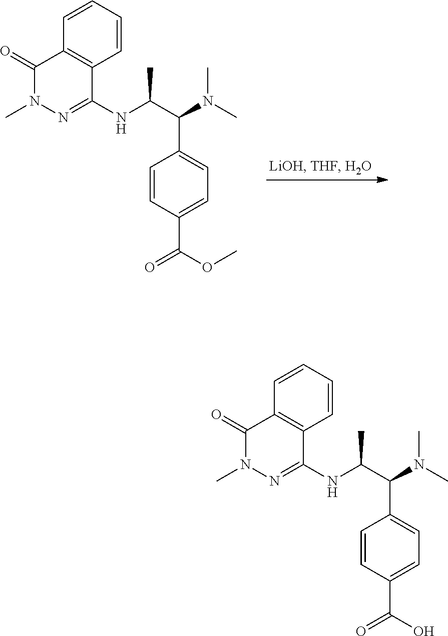 [SMILES: COC(=O)c1ccc([C@@H]([C@H](C)Nc2nn(C)c(=O)c3ccccc23)N(C)C)cc1 ; C[C@H](Nc1nn(C)c(=O)c2ccccc12)[C@H](c1ccc(C(=O)O)cc1)N(C)C]

To a stirred solution of methyl 4-((1S,2S)-1-(dimethylamino)-2-((3-methyl-4-oxo-3,4-dihydrophthalazin-1-yl)amino)propyl)benzoate (0.28 g, 0.70 mmol, from step 3) in methanol (3 mL), tetrahydrofuran (3 mL) and water (1 mL) was added lithium hydroxide monohydrate (0.02 g, 1.08 mmol) and the reaction mixture was stirred at 80° C. for 2 h. The progress of the reaction was monitored by TLC. After completion of reaction, the reaction mixture was concentrated under reduced pressure and then acidified using 1N HCl, further concentrated under reduced pressure followed by trituration with diethyl ether and heptane to afford 4-((1S,2S)-1-(dimethylamino)-2-((3-methyl-4-oxo-3,4-dihydrophthalazin-1-yl)amino)propyl)-benzoic acid (0.18 g, 67%) as white solid. LCMS: m/z 381.15 [M+H]⁺.

### Step 5: 3-(4-(9-(4-((1S,2S)-1-(dimethylamino)-2-((3-methyl-4-oxo-3,4-dihydrophthala-zin-1-yl)amino)propyl)benzoyl)-3,9-diazaspiro[5.5]undecan-3-yl)phenyl)piperidine-2,6-dione

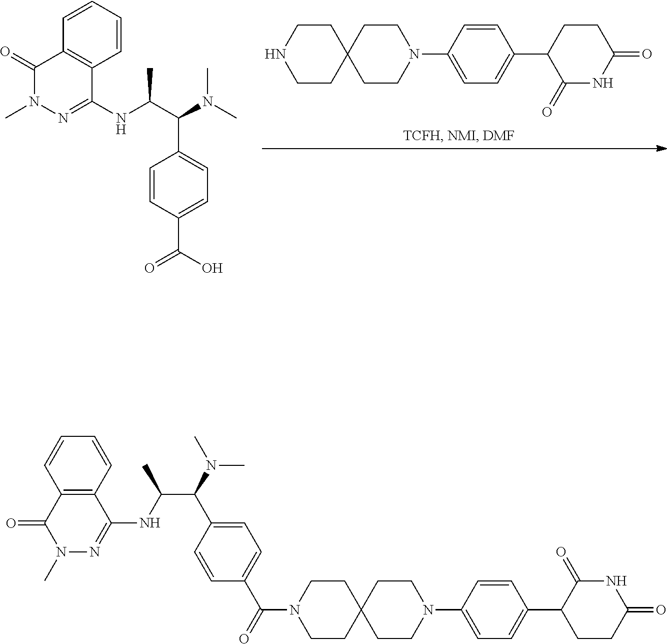

To a stirred solution of 3-[4-(3,9-diazaspiro[5.5]undecan-3-yl)phenyl]piperidine-2,6-dione (0.10 g, 0.21 mmol, from general intermediate 22) in DMF (3 mL) was added 1-methylimidazole (0.09 g, 1.10 mmol) and the reaction mixture was stirred at RT for 10 min. Added 4-((1S,2S)-1-(dimethylamino)-2-((3-methyl-4-oxo-3,4-dihydrophthalazin-1-yl)amino)propyl)benzoic acid (0.10 g, 0.26 mmol, from step-4) followed by TCFH (0.09 g, 0.32 mmol) and the reaction mixture was stirred at RT for 16 h. The progress of the reaction was monitored by TLC and LCMS. After completion of the reaction, the reaction mixture was diluted with ice-cold water and extracted with 10% MeOH in DCM. The combined organic layer was dried over anhydrous sodium sulphate, filtered, and concentrated under reduced pressure to afford crude compound. The crude was purified by preparative HPLC (Column: X-bridge (250*30 mm), 5μ, Mobile Phase A: 10 mM ABC in water, Mobile Phase B: 100% ACN) to afford 3-(4-(9-(4-((1S,2S)-1-(dimethylamino)-2-((3-methyl-4-oxo-3,4-dihydrophthalazin-1-yl)amino)propyl)benzoyl)-3,9-diazaspiro[5.5]-undecan-3-yl)phenyl)piperidine-2,6-dione (0.08 g, 56%) as a white solid. ¹H NMR (400 MHz, DMSO-d₆) δ=10.75 (s, 1H), 8.27 (dd, J=1.3, 7.9 Hz, 1H), 8.04 (d, J=7.8 Hz, 1H), 7.92-7.81 (m, 2H), 7.38 (d, J=8.0 Hz, 2H), 7.28 (d, J=8.0 Hz, 2H), 7.03 (d, J=8.8 Hz, 2H), 6.89 (d, J=8.8 Hz, 2H), 6.27 (d, J=4.4 Hz, 1H), 4.45-4.39 (m, 1H), 3.74-3.62 (m, 7H), 3.38-3.35 (m, 1H), 3.14 (br s, 4H), 2.62-2.57 (m, 1H), 2.47-2.40 (m, 3H), 2.11 (s, 6H), 2.05-1.97 (m, 1H), 1.65-1.59 (m, 4H), 1.49 (br s, 4H), 1.04 (d, J=6.4 Hz, 3H). LCMS: m/z 704.63 [M+H]⁺.

### Example 22: 3-(6-((4-((3R,5R)-5-((5-bromo-1-methyl-6-oxo-1,6-dihydropyridazin-4-yl)amino)-1-methylpiperidin-3-yl)benzyl)oxy)-1-oxoisoquinolin-2(1H)-yl)piperidine-2,6-dione (Compound 433)

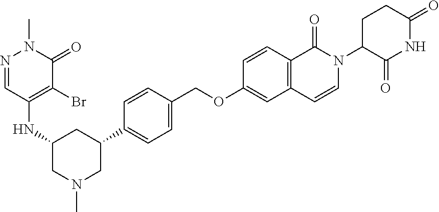 [SMILES: CN1C[C@H](Nc2cnn(C)c(=O)c2Br)C[C@H](c2ccc(COc3ccc4c(=O)n(C5CCC(=O)NC5=O)ccc4c3)cc2)C1]

### Step 1: Methyl 2-bromo-4-methoxybenzoate

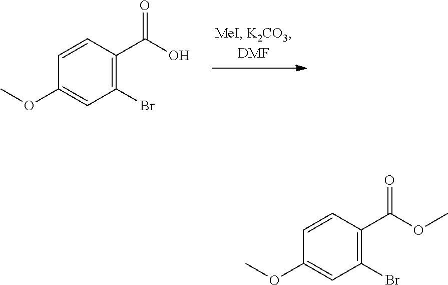

To a stirred solution of 2-bromo-4-methoxybenzoic acid (5.0 g, 22.00 mmol) in DMF (50.0 mL) was added potassium carbonate (5.97 g, 42.80 mmol) and the reaction mixture was stirred at 0° C. for 10 min. Iodomethane (2.02 mL) was added to the reaction mixture and the reaction mixture was stirred at RT for 1 h. The progress of the reaction was monitored by TLC. After completion of reaction, the reaction mixture was diluted with water and extracted with ethyl acetate. The organic layer was then washed with water, and brine solution, respectively, dried over anhydrous sodium sulphate, filtered, and concentrated under reduced pressure to afford methyl 2-bromo-4-methoxybenzoate (5.10 g, 96%) as a semi solid. The crude was used as such to the next step without further purification. LC-MS: m/z 244.90 [M+H]⁺.

### Step 2: Methyl (E)-2-(2-ethoxyvinyl)-4-methoxybenzoate

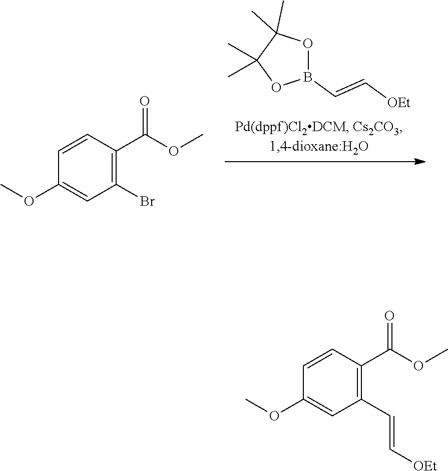

To a stirred solution of methyl 2-bromo-4-methoxybenzoate (5.0 g, 20.00 mmol, from step 1) in 1,4-dioxane (50 mL) and water (5 mL) were added cesium carbonate (22.6 g, 68.7 mmol) and (E)-2-(2-ethoxyvinyl)-4,4,5,5-tetramethyl-1,3,2-dioxaborolane (4.60 g, 23.00 mmol), respectively, and purged with nitrogen for 10 min. Then 1,1′-bis(diphenylphosphino)ferrocene-palladium(II)dichloride dichloromethane complex (1.90 g, 2.30 mmol) was added and the reaction mixture was stirred at RT for 16 h. The progress of the reaction was monitored by TLC. After completion of the reaction, the reaction mixture was filtered on a celite pad and washed with ethyl acetate. The combined filtrate was washed with water, and brine solution, respectively, dried over anhydrous sodium sulphate, filtered, and concentrated under reduced pressure to afford methyl (E)-2-(2-ethoxyvinyl)-4-methoxybenzoate (5.0 g crude) as a brown semi solid which was used as such to the next step. LC-MS: m/z 237.10 [M+H]⁺.

### Step 3: (E)-2-(2-ethoxyvinyl)-4-methoxybenzoic acid

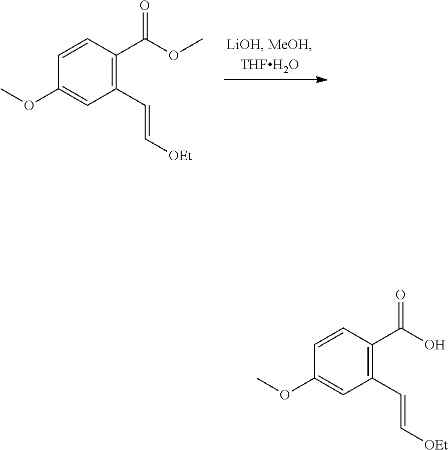

To a stirred solution of methyl (E)-2-(2-ethoxyvinyl)-4-methoxybenzoate (5 g, 21 mmol, from step 2) in THF (15 mL), methanol (5 mL) and water (5.0 mL) was added lithium hydroxide (3 g, 130 mmol) at 0° C. The reaction mixture was stirred at 55° C. for 16 h. The progress of the reaction was monitored by TLC. After completion of reaction, the reaction mixture was concentrated under reduced pressure to obtain crude product, which was diluted with water, washed with diethyl ether and then acidified with 2N HCl solution. The precipitated solids were filtered and washed with heptane to afford (E)-2-(2-ethoxyvinyl)-4-methoxybenzoic acid (2.5 g, 53%) as a pale brown solid. LC-MS: m/z 222.96 [M+H]⁺.

### Step 4: (E)-N-(2,6-dioxopiperidin-3-yl)-2-(2-ethoxyvinyl)-4-methoxybenzamide

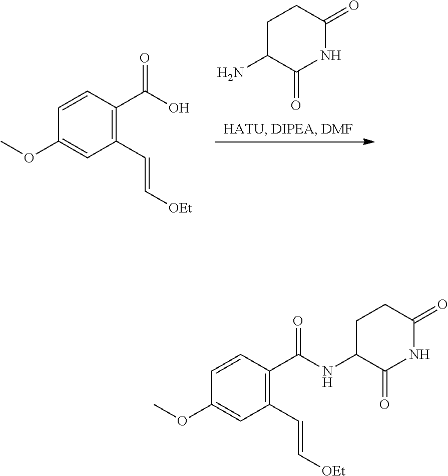 [SMILES: CCO/C=C/c1cc(OC)ccc1C(=O)O ; NC1CCC(=O)NC1=O ; CCO/C=C/c1cc(OC)ccc1C(=O)NC1CCC(=O)NC1=O]

To a stirred solution of (E)-2-(2-ethoxyvinyl)-4-methoxybenzoic acid (1.0 g, 4.50 mmol, from step 3) in DMF (10.00 mL), were added N,N-diisopropylethylamine (2.5 mL) and 3-aminopiperidine-2,6-dione (0.89 g, 6.94 mmol) and stirred at 0° C. for 10 min. HATU (2.56 g, 6.67 mmol) was added to the reaction mixture and the reaction mixture was stirred at RT for 16 h. The progress of the reaction was monitored by TLC. After completion of the reaction, the reaction mixture was diluted with water and extracted with ethyl acetate. The organic layer was washed with water followed by brine solution. The organic layer was dried over anhydrous sodium sulphate, filtered, and concentrated under reduced pressure to obtain crude product. The crude compound was triturated with 80% ethyl acetate in heptane to afford (E)-N-(2,6-dioxopiperidin-3-yl)-2-(2-ethoxyvinyl)-4-methoxybenzamide (1.3 g, 87%) as a brown solid. LC-MS: m/z 333.10 [M+H]⁺.

### Step 5: 3-(6-methoxy-1-oxoisoquinolin-2 (1H)-yl)piperidine-2,6-dione

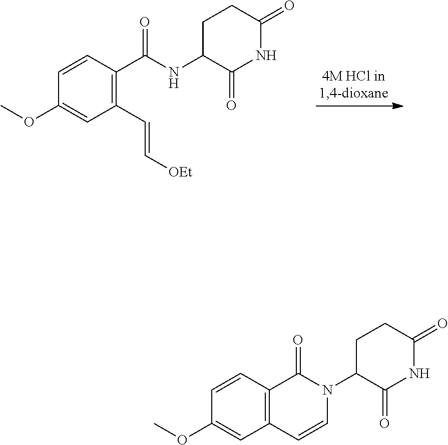 [SMILES: CCO/C=C/c1cc(OC)ccc1C(=O)NC1CCC(=O)NC1=O ; COc1ccc2c(=O)n(C3CCC(=O)NC3=O)ccc2c1]

To a stirred solution of €-N-(2,6-dioxopiperidin-3-yl)-2-(2-ethoxyvinyl)-4-methoxybenzamide (3.0 g, 9.03 mmol, from step 4) in 1,4-dioxane (30 mL), was added 4 M HCl in dioxane (1.5 mL) and the reaction mixture was stirred at 0° C. for 10 min. The reaction mixture was stirred at RT for 16 h. The progress of the reaction was monitored by TLC. After completion of the reaction, the reaction mixture was concentrated, diluted with water, and basified with aq. sodium bicarbonate solution. The aqueous layer was extracted with ethyl acetate and the organic layer was washed with brine solution. The organic layer was concentrated under reduced pressure to get crude compound. The crude compound was triturated with 50% ethyl acetate in heptane to afford 3-(6-methoxy-1-oxoisoquinolin-2(1H)-yl)piperidine-2,6-dione (0.80 g, 31%) as a brown solid. LC-MS: m/z 287.00 [M+H]⁺.

### Step 6: 3-(6-hydroxy-1-oxoisoquinolin-2(1H)-yl)piperidine-2,6-dione

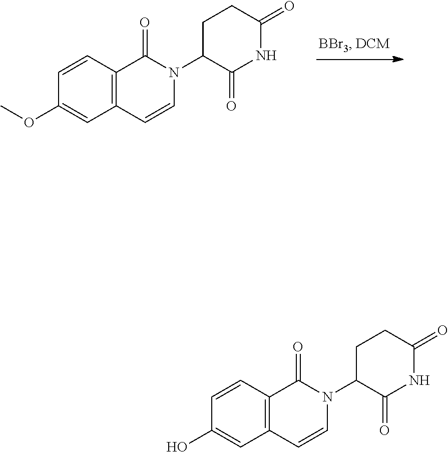 [SMILES: COc1ccc2c(=O)n(C3CCC(=O)NC3=O)ccc2c1 ; O=C1CCC(n2ccc3cc(O)ccc3c2=O)C(=O)N1]

To a stirred solution of 3-(6-methoxy-1-oxoisoquinolin-2(1H)-yl)piperidine-2,6-dione (0.30 g, 1.04 mmol, form step 5) in DCM (10.0 mL) was added boron tribromide, 1 M in DCM (3.5 mL) at 0° C. and the reaction mixture was stirred at 0° C. for 10 min. The reaction mixture was stirred at RT for 16 h. The progress of the reaction was monitored by TLC. After completion of the reaction, the reaction mixture was filtered on a celite pad and washed with ethyl acetate. The combined organic layer was concentrated under reduced pressure to get crude compound. The crude was purified by flash chromatography (silica gel, 230-400 mesh) using 10% MeOH in DCM as eluant to afford 3-(6-hydroxy-1-oxoisoquinolin-2 (1H)-yl)piperidine-2,6-dione (0.11 g, 39%) as a brown solid. LC-MS: m/z 272.90 [M+H]⁺.

### Step 7: 3-(6-((4-((3R,5R)-5-((5-bromo-1-methyl-6-oxo-1,6-dihydropyridazin-4-yl)amino)-1-methylpiperidin-3-yl)benzyl)oxy)-1-oxoisoquinolin-2(1H)-yl)piperidine-2,6-dione

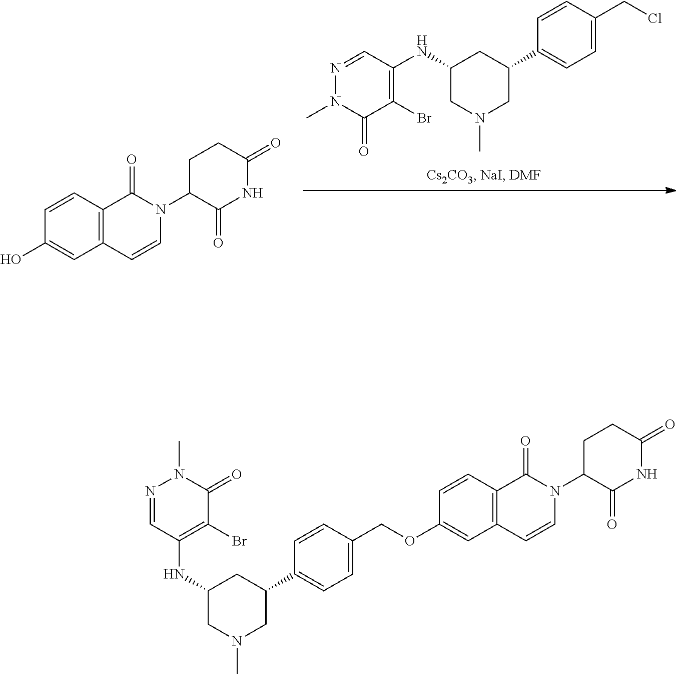

To a stirred solution of 3-(6-hydroxy-1-oxoisoquinolin-2 (1H)-yl)piperidine-2,6-dione (0.10 g; 0.36 mmol, from step 6) in DMF (3 mL) was added cesium carbonate (0.36 g, 1.10 mmol) and stirred at RT for 10 min. Then 3-(6-((4-((3R,5R)-5-((5-bromo-1-methyl-6-oxo-1,6-dihydropyridazin-4-yl)amino)-1-methylpiperidin-3-yl)benzyl)oxy)-1-oxoisoquinolin-2 (1H)-yl)piperidine-2,6-dione (0.18 g, 0.43 mmol, from general intermediate 26-28) was added and sodium iodide (0.01 g, 0.03 mmol) to the reaction mixture and the reaction mixture was stirred at 80° C. for 16 h. The progress of the reaction was monitored by LCMS. After completion of the reaction, the reaction mixture was filtered on a celite pad and washed with DCM. The combined filtrate was concentrated under reduced pressure to get crude compound, which was first partially purified by silica gel column chromatography using 5-10% MeOH in DCM eluent and the obtained partially pure solid was further purified by preparative HPLC [Column: XTIMATE C18 (250*21.2 mm) 5μ; Mobile phase A: 10 mM ammonium bicarbonate in water, Mobile phase B: 100% acetonitrile] to afford 3-(6-(4-(3R,5R)-5-(5-bromo-1-methyl-6-oxo-1,6-dihydropyridazin-4-yl)amino)-1-methylpiperidin-3-yl)benzyl)oxy)-1-oxoisoquinolin-2 (1H)-yl)piperidine-2,6-dione (0.004 g, 2.3%) as an off white solid. ¹H NMR (400 MHz, DMSO-d₆) δ=11.02 (br s, 1H), 8.10 (d, J=8.8 Hz, 1H), 7.88 (s, 1H), 7.44 (d, J=8.2 Hz, 2H), 7.38 (d, J=7.6 Hz, 1H), 7.30 (d, J=8.4 Hz, 2H), 7.24 (d, J=2.4 Hz, 1H), 7.15 (dd, J=8.8, 2.4 Hz, 1H), 6.58 (d, J=7.2 Hz, 1H), 5.82 (d, J=9.2 Hz, 1H), 5.58-5.32 (br s, 1H), 5.21 (s, 2H), 4.00-3.92 (m, 1H), 3.59 (s, 3H), 2.98-2.92 (m, 2H), 2.85-2.82 (m, 2H), 2.64-2.57 (m, 2H), 2.24 (s, 3H), 2.06-1.87 (m, 4H), 1.72 (q, J=12.0 Hz, 1H). LC-MS: m/z 659.38 [M−H]⁻.

### Example 23: 3-(3-((1-(4-((3R,5R)-5-((5-bromo-1-methyl-6-oxo-1,6-dihydropyridazin-4-yl)amino)-1-methylpiperidin-3-yl)phenyl)piperidin-4-yl)oxy)phenyl)piperidine-2,6-dione (Compound 434)

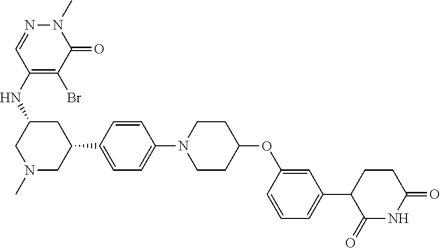 [SMILES: CN1C[C@H](Nc2cnn(C)c(=O)c2Br)C[C@H](c2ccc(N3CCC(Oc4cccc(C5CCC(=O)NC5=O)c4)CC3)cc2)C1]

### Step 1: 3-(2,6-bis(benzyloxy)pyridin-3-yl)phenol

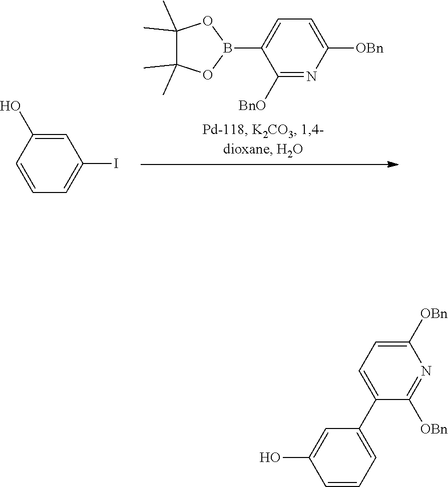 [SMILES: CC1(C)OB(c2ccc(OCc3ccccc3)nc2OCc2ccccc2)OC1(C)C ; Oc1cccc(I)c1 ; Oc1cccc(-c2ccc(OCc3ccccc3)nc2OCc2ccccc2)c1]

To a stirred solution of 3-iodophenol (4 g, 18.18 mmol) and 2,6-dibenzyloxy-3-(4,4.5,5-tetramethyl-1,3,2-dioxaborolan-2-yl)pyridine (7.58 g, 18.20 mmol) in 1,4-dioxane (40 mL) and water (10 mL), was added potassium carbonate (7.52 g, 53.90 mmol) and the reaction mixture was purged with argon for 20 min. To the resulting reaction mixture, PdCl₃(dtbpf) (1.17 g, 1.76 mmol) was added, and the reaction mixture was again purged with argon for 10 min. The reaction mixture was stirred at 100° C. for 16 h in a sealed tube. The progress of the reaction was monitored by TLC and LCMS. After completion of reaction, the reaction mixture was filtered through a celite pad, and the filtrate was concentrated under reduced pressure to afford crude compound. The crude was purified by silica gel column chromatography using 80% EtOAc in heptane as eluent to afford 3-(2,6-bis(benzyloxy)pyridin-3-yl)phenol (4 g, 57%) as a sticky semi solid. LC-MS: m/z 384.20 [M+H]⁺.

### Step 2: tert-butyl 4-(3-(2,6-bis(benzyloxy)pyridin-3-yl)phenoxy) piperidine-1-carboxylate

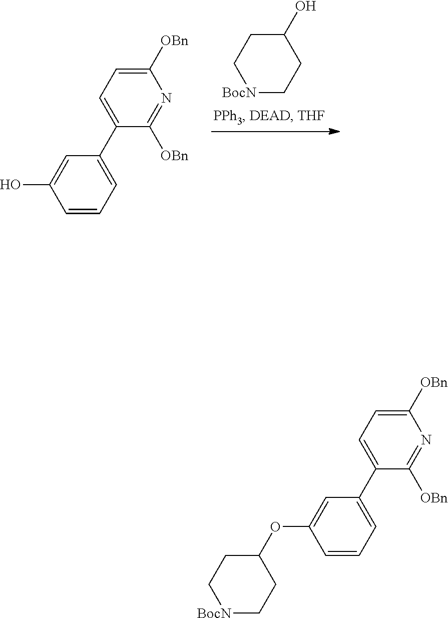 [SMILES: Oc1cccc(-c2ccc(OCc3ccccc3)nc2OCc2ccccc2)c1 ; CC(C)(C)OC(=O)N1CCC(O)CC1 ; CC(C)(C)OC(=O)N1CCC(Oc2cccc(-c3ccc(OCc4ccccc4)nc3OCc3ccccc3)c2)CC1]

To a stirred solution of 3-(2,6-dibenzyloxy-3-pyridyl)phenol (3 g, 7.82 mmol) in tetrahydrofuran (30 mL), was added tert-butyl 4-hydroxypiperidine-1-carboxylate (1.47 g, 7.30 mmol, from step 1) and the reaction mixture was cooled to 0° C. Then triphenylphosphine (2.4 g, 9.0 mmol) followed by diethyl azodicarboxylate (1.65 mL, 8.79 mmol) was added to the reaction mixture and the reaction mixture was stirred at RT for 16 h. The progress of the reaction was monitored by TLC. After completion of reaction, the reaction mixture was diluted with water and extracted with ethyl acetate. The combined organic layer was dried over anhydrous Na₂SO₄ and concentrated under reduced pressure. The crude compound was purified by flash chromatography (silica gel, 230-400 mesh) where the product was eluted with 30% ethyl acetate/heptane to afford tert-butyl 4-(3-(2,6-bis(benzyloxy)pyridin-3-yl)phenoxy piperidine-1-carboxylate (1.789 g, 40%) as a white semi solid. LC-MS: m/z 567.00 [M+H]⁺.

### Step 3: 2,6-bis(benzyloxy)-3-(3-(piperidin-4-yloxy)phenyl)pyridine

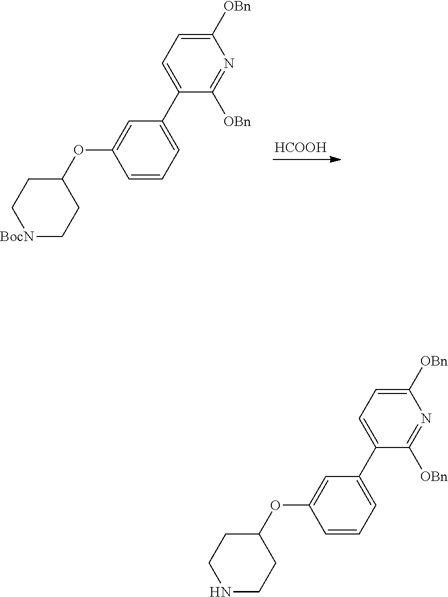 [SMILES: CC(C)(C)OC(=O)N1CCC(Oc2cccc(-c3ccc(OCc4ccccc4)nc3OCc3ccccc3)c2)CC1 ; "HCOOH" ; c1ccc(COc2ccc(-c3cccc(OC4CCNCC4)c3)c(OCc3ccccc3)n2)cc1]

A mixture of tert-butyl 4-(3-(2,6-bis(benzyloxy)pyridin-3-yl)phenoxy)piperidine-1-carboxylate (1.79 g, 3.16 mmol, from step 2) and formic acid (5 mL) was stirred at 0° C. for 5 min followed by at RT for 3 h. The progress of the reaction was monitored by TLC. After completion of reaction, the reaction mixture was concentrated under reduced pressure to afford 2,6-bis(benzyloxy)-3-(3-(piperidin-4-yloxy)phenyl)pyridine (1.450 g, 98%, formate salt) as white solid. LC-MS: m/z 467.74 [M+H]⁺.

### Step 4: tert-butyl ((3R,5R)-5-(4-(4-(3-(2,6-bis(benzyloxy)pyridin-3-yl)phenoxy)piperidin-1-yl)phenyl)-1-methylpiperidin-3-yl)carbamate

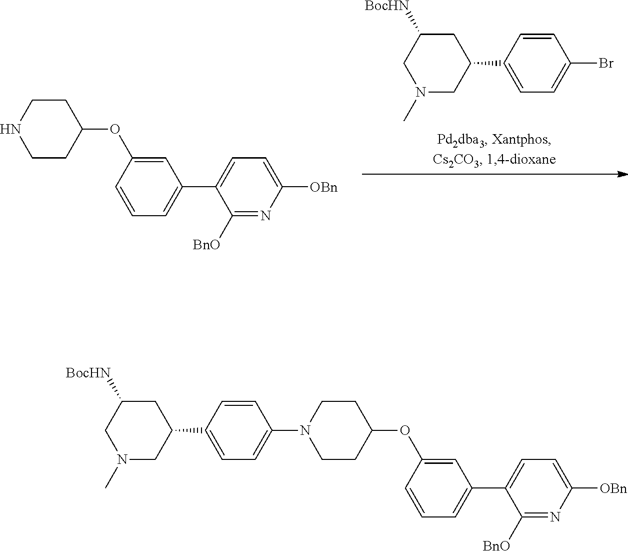 [SMILES: c1ccc(COc2ccc(-c3cccc(OC4CCNCC4)c3)c(OCc3ccccc3)n2)cc1 ; CN1C[C@H](NC(=O)OC(C)(C)C)C[C@H](c2ccc(Br)cc2)C1 ; CN1C[C@H](NC(=O)OC(C)(C)C)C[C@H](c2ccc(N3CCC(Oc4cccc(-c5ccc(OCc6ccccc6)nc5OCc5ccccc5)c4)CC3)cc2)C1]

To the stirred solution of tert-butyl ((3R,5R)-5-(4-bromophenyl)-1-methylpiperidin-3-yl)carbamate (0.35 g, 0.96 mmol, from step 6 of example 25) and 2,6-dibenzyloxy-3-[3-(4-piperidyloxy)phenyl]pyridine (0.45 g, 0.96 mmol, formate salt, from step 3) in 1,4-dioxane (10 mL), cesium carbonate (0.94 g, 2.89 mmol) was added and purged with argon gas for 15 min. Then 4,5-bis(diphenylphosphino)-9,9-dimethylxanthene (0.11 g, 0.19 mmol) and Pd₂(dba)₃ (0.09 g, 0.09 mmol) were added to the reaction mixture under argon atmosphere and further purged with argon gas for 15 min. The reaction mixture was stirred at 110° C. for 16 h. The progress of the reaction was monitored by TLC. After completion of the reaction, the reaction mixture was passed through a celite pad and the filtrate was concentrated and purified by silica gel column chromatography where the product was eluted at 20% EtOAc in heptane to afford tert-butyl ((3R,5R)-5-(4-(4-(3-(2,6-bis(benzyloxy)pyridin-3-yl)phenoxy)piperidin-1-yl)phenyl)-1-methylpiperidin-3-yl)carbamate (0.45 g, 51%) as an off-white solid. LC-MS: m/z 755.30 [M+H]⁺.

### Step 5: tert-butyl ((3R,5R)-5-(4-(4-(3-(2,6-dioxopiperidin-3-yl)phenoxy)piperidin-1-yl)phenyl)-1-methylpiperidin-3-yl)carbamate

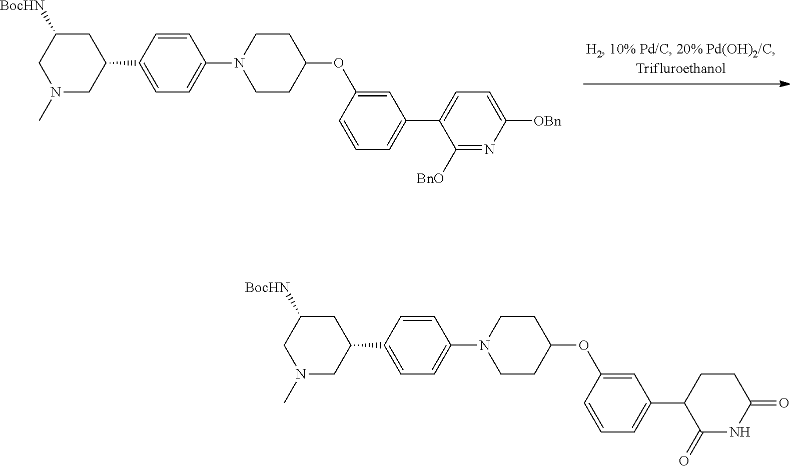 [SMILES: CN1C[C@H](NC(=O)OC(C)(C)C)C[C@H](c2ccc(N3CCC(Oc4cccc(-c5ccc(OCc6ccccc6)nc5OCc5ccccc5)c4)CC3)cc2)C1 ; CN1C[C@H](NC(=O)OC(C)(C)C)C[C@H](c2ccc(N3CCC(Oc4cccc(C5CCC(=O)NC5=O)c4)CC3)cc2)C1]

To a stirred solution of tert-butyl ((3R,5R)-5-(4-(4-(3-(2,6-dioxopiperidin-3-yl)phenoxy)piperidin-1-yl)phenyl)-1-methylpiperidin-3-yl)carbamate (0.40 g, 0.43 mmol, from step 4) in 2,2,2-trifluoroethanol (20 mL) were added 10% palladium on carbon (0.10 g) and 20% palladium hydroxide on carbon (0.10 g), respectively. The reaction mixture was stirred under hydrogen atmosphere (100 psi) at RT for 16 h in a steel bomb. The progress of the reaction was monitored by TLC and LCMS. After completion of the reaction, the reaction mixture was filtered through a celite pad and the filtrate was concentrated to afford tert-butyl ((3R,5R)-5-(4-(4-(3-(2,6-dioxopiperidin-3-yl)phenoxy)piperidin-1-yl)phenyl)-1-methyl-piperidin-3-yl)carbamate (0.19 g, 70%) as a brown semi solid. LC-MS: m/z 577.40 [M+H]⁺.

### Step 6: 3-(3-((1-(4-((3R,5R)-5-amino-1-methylpiperidin-3-yl)phenyl)piperidin-4-yl)oxy)-phenyl)piperidine-2,6-dione

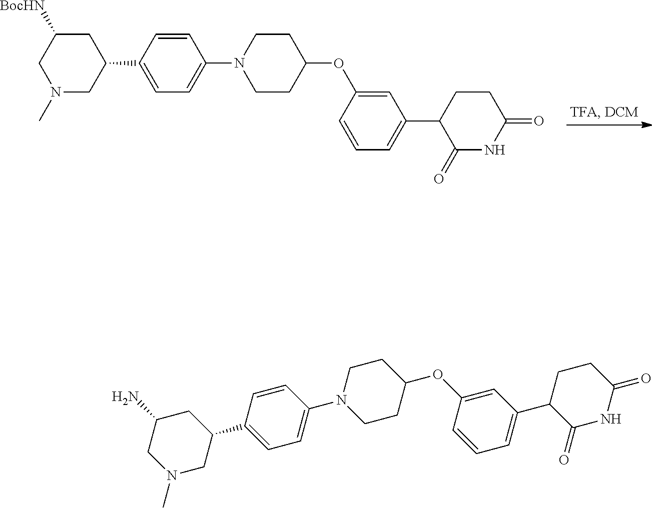 [SMILES: CN1C[C@H](NC(=O)OC(C)(C)C)C[C@H](c2ccc(N3CCC(Oc4cccc(C5CCC(=O)NC5=O)c4)CC3)cc2)C1 ; CN1C[C@H](N)C[C@H](c2ccc(N3CCC(Oc4cccc(C5CCC(=O)NC5=O)c4)CC3)cc2)C1]

To a stirred solution of tert-butyl ((3R,5R)-5-(4-(4-(3-(2,6-dioxopiperidin-3-yl)phenoxy)piperidin-1-yl)phenyl)-1-methylpiperidin-3-yl)carbamate (0.19 g, 0.30 mmol, from step 5) in DCM (20 mL) was added TFA (1 mL) at 0° C. and the reaction mixture was stirred at RT for 2 h. After completion of the reaction (monitored by TLC), the reaction mixture was concentrated and triturated with ether to afford 3-(3-((1-(4-((3R,5R)-5-amino-1-methylpiperidin-3-yl)phenyl)piperidin-4-yl)oxy)phenyl)-piperidine-2,6-dione (0.18 g, 98%, TFA salt) as an off-white solid. LC-MS: m/z 477.40 [M+H]⁺.

### Step 7: 3-(3-((1-(4-((3R,5R)-5-((5-bromo-1-methyl-6-oxo-1,6-dihydropyridazin-4-yl)amino)-1-methylpiperidin-3-yl)phenyl)piperidin-4-yl)oxy)phenyl)piperidine-2,6-dione

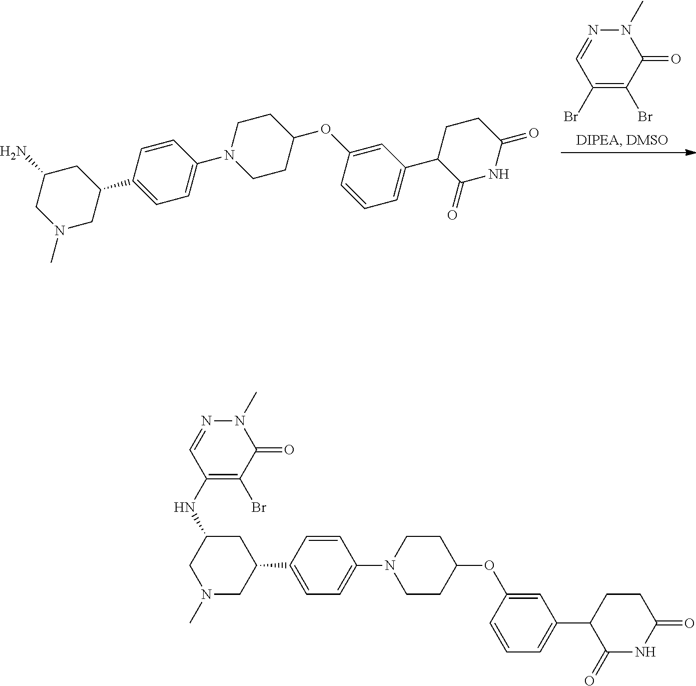

To the stirred solution of 3-[3-[[1-[4-[(3~{R})-5-amino-1-methyl-3-piperidyl]phenyl]-4-piperidyl]oxy]phenyl]piperidine-2,6-dione (0.18 g, 0.37 mmol, from step 6) and 4,5-dibromo-2-methylpyridazin-3-one (0.12 g, 0.45 mmol) in DMSO (2 mL) was added N,N-diisopropylethylamine (0.15 g, 1.13 mmol) and the reaction mixture was stirred at 120° C. for 2 h. The progress of the reaction was monitored by TLC. After completion of the reaction, the reaction mixture was concentrated under reduced pressure to obtain crude compound, which was purified by preparative HPLC [Column: Synergy C18 (150*20 mm) 5 μm, Mobile Phase A: 0.1% FA in water, Mobile Phase B: 100% can to afford 3-(3-((1-(4-((3R,5R)-5-((5-bromo-1-methyl-6-oxo-1,6-dihydropyridazin-4-yl)amino)-1-methylpiperidin-3-yl)phenyl)piperidin-4-yl)oxy)phenyl)piperidine-2,6-dione (0.005 g, 2%) as an off-white solid. ¹H NMR (400 MHz, DMSO-d₆) δ=10.80 (s, 1H), 7.86 (s, 1H), 7.23 (t, J=8.0 Hz, 1H), 7.10 (d, J=8.4 Hz, 2H), 6.93-6.83 (m, 4H), 6.78 (d, J=7.6 Hz, 1H), 5.79 (d, J=9.2 Hz, 1H), 4.58-4.51 (m, 1H), 4.05-3.89 (m, 1H), 3.84-3.79 (m, 1H), 3.60-3.54 (m, 3H), 3.51-3.44 (m, 2H), 3.06-2.96 (m, 2H), 2.95-2.76 (m, 3H), 2.69-2.60 (m, 1H), 2.26-2.17 (m, 4H), 2.07-1.99 (m, 4H), 1.93-1.80 (m, 2H), 1.75-1.62 (m, 3H). Note: Compound present as formic acid salt. One aliphatic proton is merged with solvent peak. LC-MS: m/z 663.20 [M+H]⁺.

### Example 24: 3-(3-(4-(4-((3R,5R)-5-((5-bromo-1-methyl-6-oxo-1,6-dihydropyridazin-4-yl)amino)-1-methylpiperidin-3-yl)benzyl)piperazin-1-yl)phenoxy)piperidine-2,6-dione (Compound 435)

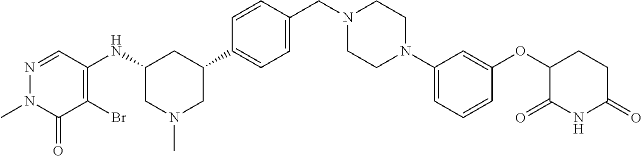 [SMILES: CN1C[C@H](Nc2cnn(C)c(=O)c2Br)C[C@H](c2ccc(CN3CCN(c4cccc(OC5CCC(=O)NC5=O)c4)CC3)cc2)C1]

### Step 1: tert-butyl 4-(3-hydroxyphenyl)piperazine-1-carboxylate

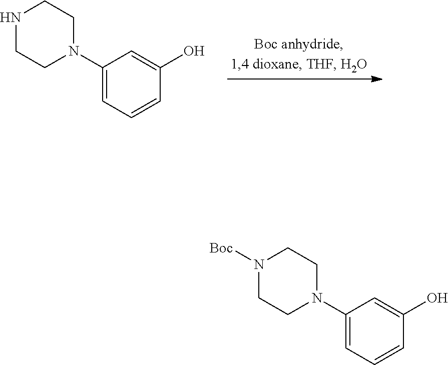 [SMILES: Oc1cccc(N2CCNCC2)c1 ; CC(C)(C)OC(=O)N1CCN(c2cccc(O)c2)CC1]

To a stirred solution of 3-(piperazin-1-yl) phenol (2 g, 11.22 mmol) in 1,4-dioxane (30 mL) and water (30 mL), di-tert-butyl dicarbonate (3 mL) was added to the reaction mixture at RT followed by addition of THF (30 mL) and sodium bicarbonate (2 g, 23.7 mmol). The reaction mixture was stirred at RT for 12 h. The progress of the reaction was monitored by TLC. After completion of the reaction, the reaction mixture was extracted with ethyl acetate. The combined organic layer was dried over anhydrous Na₂SO₄, filtered and concentrated under reduced pressure to obtain crude compound. The crude was purified by silica gel column chromatography to afford tert-butyl 4-(3-hydroxyphenyl)piperazine-1-carboxylate (2.2 g, 70%) as white solid. LCMS: m/z 279.10 [M+H]⁺.

### Step 2: tert-butyl 4-(3-((2,6-dioxopiperidin-3-yl)oxy)phenyl)piperazine-1-carboxylate

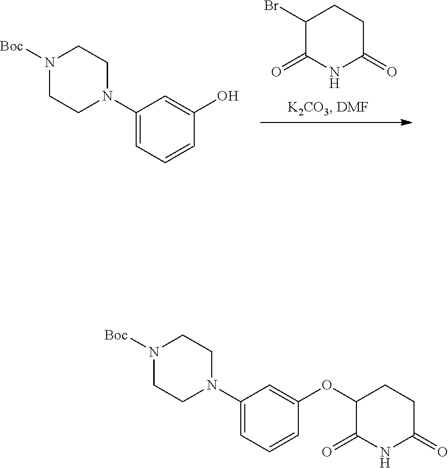 [SMILES: CC(C)(C)OC(=O)N1CCN(c2cccc(O)c2)CC1 ; O=C1CCC(Br)C(=O)N1 ; CC(C)(C)OC(=O)N1CCN(c2cccc(OC3CCC(=O)NC3=O)c2)CC1]

To a stirred solution of tert-butyl 4-(3-hydroxyphenyl)piperazine-1-carboxylate (1.2 g, 4.3 mmol, from step 1) in THF (40 mL), was added sodium hydride (60 wt % in oil, 0.75 g, 19 mmol) at 0° C. and stirred at 60° C. for 1 h. Then 3-bromopiperidine-2,6-dione (1.8 g, 9.4 mmol) was added to the reaction mixture and continued stirring at 60° C. for 1 h. The progress of the reaction was monitored by TLC. The reaction mixture was diluted with aq. NaHCO₃ solution and extracted with DCM. The combined organic layer was dried over anhydrous Na₂SO₄, filtered and concentrated under reduced pressure to afford crude compound. The crude compound was purified by silica gel column chromatography eluting with 50% ethyl acetate in heptane to afford tert-butyl 4-(3-((2,6-dioxopiperidin-3-yl)oxy)phenyl)piperazine-1-carboxylate (1.5 g, 41%) as white solid. LCMS: m/z 390.32 [M+H]⁺.

### Step 3: 3-(3-(piperazin-1-yl)phenoxy)piperidine-2,6-dione

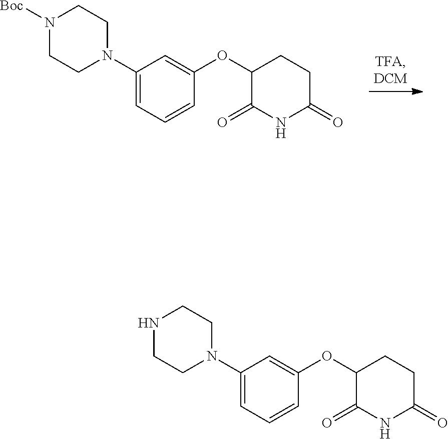 [SMILES: CC(C)(C)OC(=O)N1CCN(c2cccc(OC3CCC(=O)NC3=O)c2)CC1 ; O=C1CCC(Oc2cccc(N3CCNCC3)c2)C(=O)N1]

To a stirred solution of tert-butyl 4-(3-((2,6-dioxopiperidin-3-yl)oxy)phenyl)piperazine-1-carboxylate (0.60 g, 1.54 mmol, from step 2) in DCM (5 mL), TFA (1 mL, 13.33 mmol) was added and stirred at 0° C. for 5 min followed by at RT for 1 h. The progress of the reaction was monitored by TLC. After completion of reaction, the reaction mixture was concentrated under reduced pressure and then triturated with diethyl ether and heptane, respectively, to afford (0.4 g, 89.73%, TFA salt) as white solid. LCMS: m/z 290.27 [M+H]⁺.

### Step 4: 3-(3-(4-(4-((3R,5R)-5-((5-bromo-1-methyl-6-oxo-1,6-dihydropyridazin-4-yl)amino)-1-methylpiperidin-3-yl)benzyl)piperazin-1-yl)phenoxy)piperidine-2,6-dione

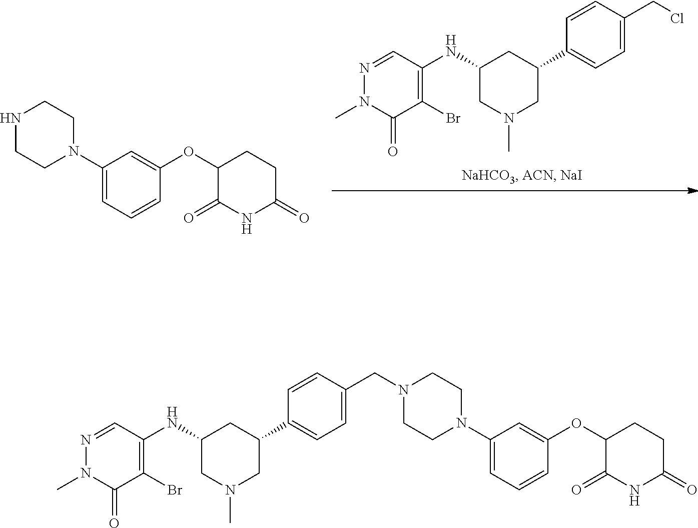 [SMILES: O=C1CCC(Oc2cccc(N3CCNCC3)c2)C(=O)N1 ; CN1C[C@H](Nc2cnn(C)c(=O)c2Br)C[C@H](c2ccc(CCl)cc2)C1 ; CN1C[C@H](Nc2cnn(C)c(=O)c2Br)C[C@H](c2ccc(CN3CCN(c4cccc(OC5CCC(=O)NC5=O)c4)CC3)cc2)C1]

To a stirred solution of 3-(3-piperazin-1-ylphenoxy) piperidine-2,6-dione (0.20 g, 0.69 mmol TFA salt, from step 3) in acetonitrile (10 mL) was added sodium bicarbonate (0.16 g, 1.90 mmol) and stirred for 20 min at RT. Then 4 Å molecular sieves and 4-bromo-5-[[(3~{R},5~{R})-5-[4-(chloromethyl)phenyl]-1-methyl-3-piperidyl]amino]-2-methyl-pyridazin-3-one (220 mg, 0.5167 mmol, general intermediate 26-28) were added to the reaction mixture and the reaction mixture was stirred at 80° C. for 1 h. The progress of the reaction was monitored by TLC. After completion of reaction, the reaction mixture was filtered. The filtrate was concentrated to obtain crude and the crude compound was purified by preparative HPLC [Column: X-BRIDGE C18 (250*30 mm) 5μ; Mobile phase A: 10 mM ammonium bicarbonate in water, Mobile phase B: 100% acetonitrile] to afford 3-(3-(4-(4-((3R,5R)-5-((5-bromo-1-methyl-6-oxo-1,6-dihydropyridazin-4-yl)amino)-1-methylpiperidin-3-yl)benzyl)piperazin-1-yl)phenoxy)piperidine-2,6-dione (0.04 g, 13%) as white solid. ¹H NMR (400 MHz, DMSO-d₆) δ=10.89 (s, 1H), 7.87 (s, 1H), 7.28-7.22 (m, 4H), 7.08 (t, J=8.5 Hz, 1H), 6.54-6.50 (m, 2H), 6.45-6.42 (m, 1H), 5.81 (d, J=8.8 Hz, 1H), 5.17 (dd, J=10.8, 5.2 Hz, 1H), 3.97-3.92 (m, 1H), 3.59 (s, 3H), 3.48 (s, 2H), 3.11 (brs, 4H), 2.96-2.91 (m, 2H), 2.83 (br d, J=10.9 Hz, 1H), 2.74-2.66 (m, 1H), 2.63-2.56 (m, 1H), 2.46-2.41 (m, 4H), 2.24 (s, 3H), 2.19-2.07 (m, 2H), 2.04-1.86 (m, 3H), 1.71 (q, J=12.0 Hz, 1H). LCMS: m/z 678.0, 680.0 [M+H]⁺.

### Example 25: 3-(4-(9-(4-((3R,5R)-5-((5-bromo-1-methyl-6-oxo-1,6-dihydropyridazin-4-yl)amino)-1-methylpiperidin-3-yl)phenyl)-3,9-diazaspiro[5.5]undecan-3-yl)-2-methylphenyl)piperidine-2,6-dione (Compound 436)

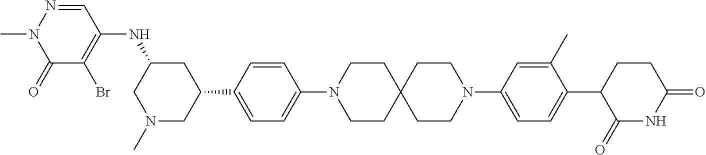 [SMILES: Cc1cc(N2CCC3(CCN(c4ccc([C@H]5C[C@@H](Nc6cnn(C)c(=O)c6Br)CN(C)C5)cc4)CC3)CC2)ccc1C1CCC(=O)NC1=O]

### Step 1: tert-butyl (R)-3-(picolinamido)piperidine-1-carboxylate

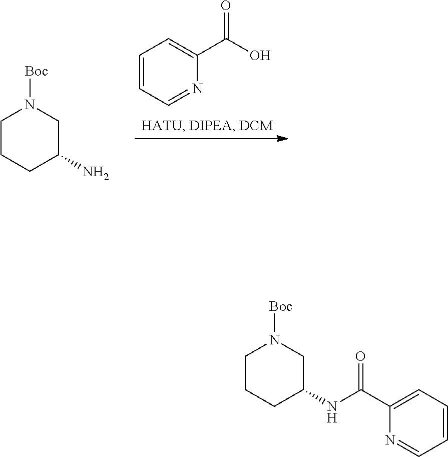 [SMILES: CC(C)(C)OC(=O)N1CCC[C@@H](N)C1 ; O=C(O)c1ccccn1 ; CC(C)(C)OC(=O)N1CCC[C@@H](NC(=O)c2ccccn2)C1]

To a stirred solution of tert-butyl (R)-3-(picolinamido)piperidine-1-carboxylate (73.8 g, 600 mmol) in DCM (1 L) was added N,N-diisopropylamine (130.9 mL, 750 mmol) at RT. The reaction mixture was stirred at RT for 10 min. To this were added tert-butyl (R)-3-aminopiperidine-1-carboxylate (100.0 g, 500 mmol) followed by HATU (228.0 g, 600 mmol) and the reaction mixture was stirred at RT for 16 h. The progress of the reaction was monitored by TLC and LCMS. After completion of reaction, the reaction mixture was diluted with ice-cold water and then filtered to obtain precipitate, which was then triturated with heptane to obtain tert-butyl (R)-3-(picolinamido)piperidine-1-carboxylate (0.12 g, 79%) as an off white solid. LC-MS: m/z 306.00 [M+H]⁺.

### Step 2: tert-butyl (3R,5R)-3-(4-bromophenyl)-5-(picolinamido)piperidine-1-carboxylate

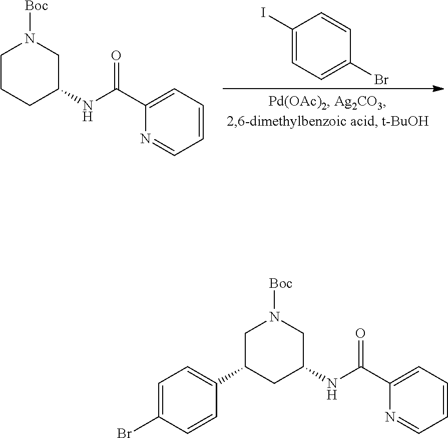 [SMILES: CC(C)(C)OC(=O)N1CCC[C@@H](NC(=O)c2ccccn2)C1 ; Brc1ccc(I)cc1 ; CC(C)(C)OC(=O)N1C[C@H](NC(=O)c2ccccn2)C[C@H](c2ccc(Br)cc2)C1]

To a stirred solution of tert-butyl (R)-3-(picolinamido)piperidine-1-carboxylate (5.00 g, 16.37 mmol, from step 1) and 1-bromo-4-iodo-benzene (27.79 g, 98.23 mmol) in tert-butanol (50.00 mL), were added 2,6-dimethylbenzoic acid (0.63 g, 4.093 mmol) and silver carbonate (4.56 g, 16.37 mmol) and the reaction mixture was purged with argon for 20 min. To the resulting reaction mixture, palladium(II) acetate (0.38 g, 1.64 mmol) was added and the reaction mixture was again purged with argon for 10 min. The reaction mixture was stirred in a sealed tube at 120° C. for 45 h. The progress of the reaction was monitored by TLC. After completion of reaction, the reaction mixture was filtered through celite. The filtrate was concentrated under reduced pressure. The crude compound was stirred at 50° C. for 30 min in heptane and filtered. The filtrate was concentrated under reduced pressure and purified by silica gel column chromatography where the product was eluted with 20% ethyl acetate in hexane to afford tort-butyl (3R,5R)-3-(4-bromophenyl)-5-(picolinamido)piperidine-1-carboxylate (6.50 g, 86%) as an off-white solid. LC-MS: m/z 461.90 [M+H]⁺.

### Step 3: N-((3R,5R)-5-(4-bromophenyl)piperidin-3-yl)picolinamide

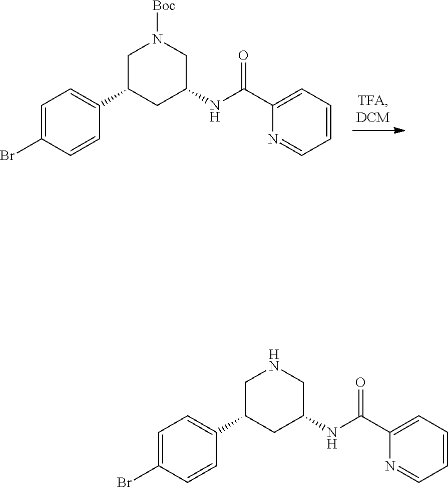 [SMILES: CC(C)(C)OC(=O)N1C[C@H](NC(=O)c2ccccn2)C[C@H](c2ccc(Br)cc2)C1 ; O=C(N[C@H]1CNC[C@@H](c2ccc(Br)cc2)C1)c1ccccn1]

To a stirred solution of tert-butyl (3R,5R)-3-(4-bromophenyl)-5-(picolinamido)piperidine-1-carboxylate (4.0 g, 8.68 mmol, from step 2) in DCM (10 mL) was added TFA (3.0 mL, 38.60 mmol) at 0° C. and the reaction mixture was stirred at RT for 2 h. The progress of the reaction was monitored by TLC. After completion of reaction, the reaction mixture was distilled under reduced pressure and then triturated with 10% methanol in DCM and diethyl ether to afford N-((3R,5R)-5-(4-bromophenyl)piperidin-3-yl) picolinamide (2.50 g, 80%, TFA salt) as an off white solid. LC-MS: m/z 361.90 [M+H]⁺.

### Step 4: N-((3R,5R)-5-(4-bromophenyl)-1-methylpiperidin-3-yl) picolinamide

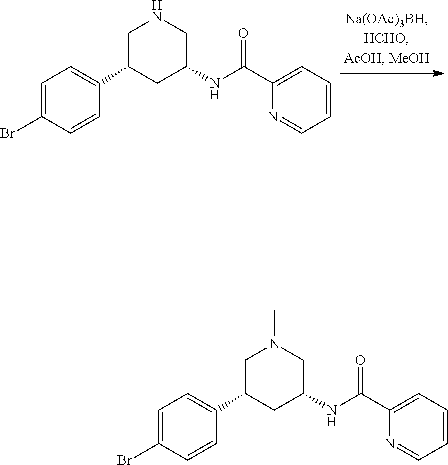 [SMILES: O=C(N[C@H]1CNC[C@@H](c2ccc(Br)cc2)C1)c1ccccn1 ; CN1C[C@H](NC(=O)c2ccccn2)C[C@H](c2ccc(Br)cc2)C1]

To a stirred solution of N-((3R,5R)-5-(4-bromophenyl)piperidin-3-yl) picolinamide (5.0 g, 13.88 mmol, from step 3) in methanol (100 mL) was added acetic acid (2 mL) at 0° C. followed by formaldehyde (37% in aqueous solution, 20.28 g, 249.90 mmol) and the reaction mixture was stirred at RT for 6 h. Then sodium triacetoxyborohydride (9.0 g, 41.19 mmol) was added to the reaction mixture and stirred at 0° C. for 16 h. The progress of the reaction was monitored by TLC. After completion of reaction, the reaction mixture was quenched with saturated sodium bicarbonate solution and the aqueous layer was extracted with ethyl acetate. The organic layer was washed with brine, dried over anhydrous sodium sulphate, filtered, and concentrated under reduced pressure to obtain crude compound. The crude was purified by silica gel column chromatography where the product was eluted with 5% MeOH in DCM to afford N-((3R,5R)-5-(4-bromophenyl)-1-methylpiperidin-3-yl) picolinamide (4.00 g, 77%) as a pale yellow solid. LC-MS: m/z 374.09 [M+H]⁺.

### Step 5: (3R,5R)-5-(4-bromophenyl)-1-methylpiperidin-3-amine

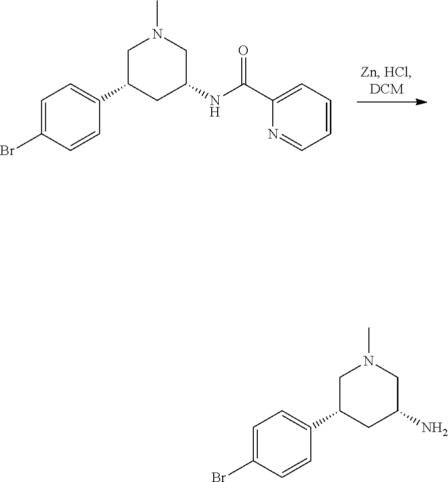 [SMILES: CN1C[C@H](NC(=O)c2ccccn2)C[C@H](c2ccc(Br)cc2)C1 ; CN1C[C@H](N)C[C@H](c2ccc(Br)cc2)C1]

To a stirred solution of N-((3R,5R)-5-(4-bromophenyl)-1-methylpiperidin-3-yl) picolinamide (4.0 g, 10.69 mmol, from step 4) in water (150 mL) were added concentrated HCl (18 mL) and zinc (10.6 g, 160 mmol) followed by DCM (150 mL) at RT and stirred at RT for 16 h. The progress of the reaction was monitored by TLC. After completion of reaction, the reaction mixture was basified with 5 M aq. NaOH solution and filtered through filter paper and extracted with DCM. The combined organic layer was washed with brine, dried over anhydrous Na₂SO₄, filtered and concentrated under reduced pressure to afford (3R,5R)-5-(4-bromophenyl)-1-methylpiperidin-3-amine (1.80 g, 63%) as a yellow liquid. LC-MS: m/z 268.90 [M+H]⁺.

### Step 6: tert-Butyl ((3R,5R)-5-(4-bromophenyl)-1-methylpiperidin-3-yl)carbamate

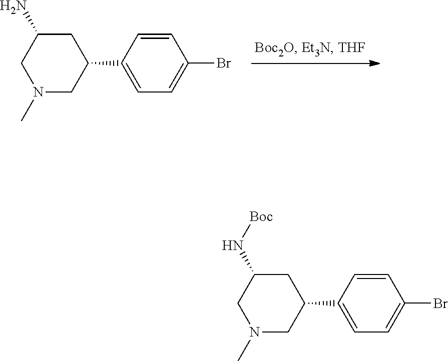 [SMILES: CN1C[C@H](N)C[C@H](c2ccc(Br)cc2)C1 ; CN1C[C@H](NC(=O)OC(C)(C)C)C[C@H](c2ccc(Br)cc2)C1]

To a stirred solution of (3R,5R)-5-(4-bromophenyl)-J-methylpiperidin-3-amine (1.70 g, 6.30 mmol, from step 5) in DCM (15 mL) were added triethylamine (2.71 mL) and di-tert-butyl 619ecarbonate (1.70 g, 7.70 mmol) at RT and stirring was continued for 16 h. The progress of the reaction was monitored by TLC. After completion of reaction, the reaction mixture was quenched with water and extracted with DCM. The organic layer was washed with brine, dried over anhydrous sodium sulphate, filtered, and concentrated under reduced pressure to obtain crude compound. The crude compound was purified by silica gel column chromatography where the product was eluted with 5% MeOH in DCM to afford tert-butyl ((3R,5R)-5-(4-bromophenyl)-1-methylpiperidin-3-yl)carbamate (1.60 g, 69%) as an off white solid. LC-MS: m/z 368.90 [M+H]⁺.

### Step 7: tert-butyl ((3R,5R)-5-(4-(9-(4-(2,6-bis(benzyloxy)pyridin-3-yl)-3-methylphenyl)-3,9-diazaspiro[5.5]undecan-3-yl)phenyl)-1-methylpiperidin-3-yl)carbamate

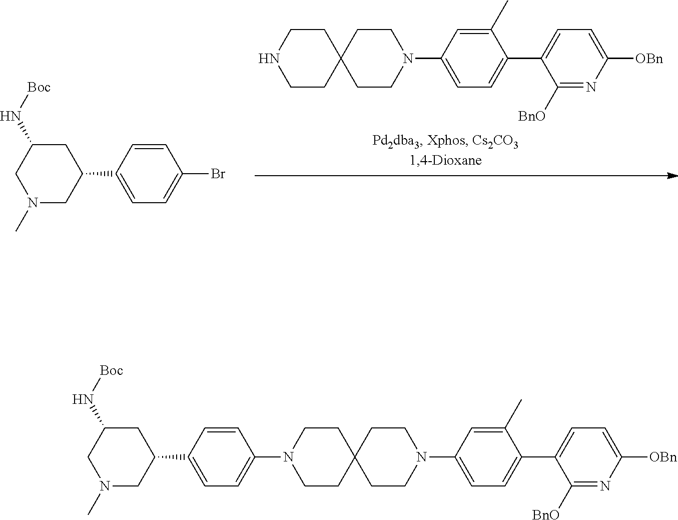 [SMILES: CN1C[C@H](NC(=O)OC(C)(C)C)C[C@H](c2ccc(Br)cc2)C1 ; Cc1cc(N2CCC3(CCNCC3)CC2)ccc1-c1ccc(OCc2ccccc2)nc1OCc1ccccc1 ; Cc1cc(N2CCC3(CCN(c4ccc([C@H]5C[C@@H](NC(=O)OC(C)(C)C)CN(C)C5)cc4)CC3)CC2)ccc1-c1ccc(OCc2ccccc2)nc1OCc1ccccc1]

To a stirred solution of tert-butyl ((3R,5R)-5-(4-bromophenyl)-1-methylpiperidin-3-yl)carbamate (0.20 g, 0.54 mmol, from step 6) and 3-(4-(2,6-bis(benzyloxy)pyridin-3-yl)-3-methylphenyl)-3,9-diazaspiro[5.5]undecane (0.34 g, 0.65 mmol, from general intermediate 22) in 1,4-dioxane (10 mL), cesium carbonate (0.53 g, 1.63 mmol) was added at RT. The reaction mixture was purged with argon for 30 min. To the resulting reaction mixture, Pd₂(dba)₃ (0.026 g, 0.02 mmol) and X-phos (0.025 g, 0.05 mmol) were added and the reaction mixture was again purged with argon for 20 min. The reaction mixture was stirred in a sealed tube at 110° C. for 16 h. The progress of the reaction was monitored by TLC. After completion of reaction, the reaction mixture was filtered through a celite pad. The filtrate was diluted with cold water and extracted with ethyl acetate. The organic layer was washed with water and brine, dried over anhydrous sodium sulphate, filtered, and concentrated under reduced pressure to afford tert-butyl ((3R,5R)-5-(4-(9-(4-(2,6-bis(benzyloxy)pyridin-3-yl)-3-methylphenyl)-3,9-diazaspiro[5.5]undecan-3-yl)phenyl)-1-methylpiperidin-3-yl)carbamate (0.07 g, 16%) as an off white solid, which was used as such for the next reaction. LC-MS: m/z 412.0 [M/2+H]⁺.

### Step 8: tert-butyl ((3R,5R)-5-(4-(9-(4-(2,6-dioxopiperidin-3-yl)-3-methylphenyl)-3,9-diazaspiro[5.5]620ndecane-3-yl)phenyl)-1-methylpiperidin-3-yl)carbamate

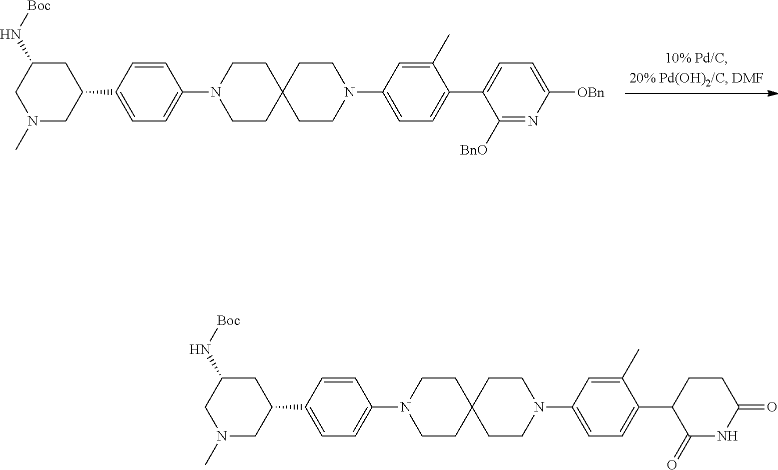 [SMILES: Cc1cc(N2CCC3(CCN(c4ccc([C@H]5C[C@@H](NC(=O)OC(C)(C)C)CN(C)C5)cc4)CC3)CC2)ccc1-c1ccc(OCc2ccccc2)nc1OCc1ccccc1 ; Cc1cc(N2CCC3(CCN(c4ccc([C@H]5C[C@@H](NC(=O)OC(C)(C)C)CN(C)C5)cc4)CC3)CC2)ccc1C1CCC(=O)NC1=O]

To a stirred of tert-butyl ((3R,5R)-5-(4-(9-(4-(2,6-bis(benzyloxy)620ndecane-3-yl)-3-methylphenyl)-3,9-diazaspiro[5.5]620ndecane-3-yl)phenyl)-1-methylpiperidin-3-yl)carbamate (0.13 g, 0.15 mmol, from step 7) in DMF (4 mL) were added 10% Pd/C (0.05 g) and 20% palladium hydroxide on carbon (0.05 g), respectively. The reaction mixture was stirred under hydrogen atmosphere (50 psi) at RT for 12 h in an autoclave. The progress of reaction was monitored by LCMS and TLC. After completion of the reaction, the reaction mixture was filtered through a celite bed and then the filtrate was concentrated under reduced pressure to obtain crude. The crude was purified by silica gel column chromatography using 10% MeOH in DCM as eluent to afford tert-butyl ((3R,5R)-5-(4-(9-(4-(2,6-dioxopiperidin-3-yl)-3-methylphenyl)-3,9-diazaspiro[5.5]undecan-3-yl)phenyl)-1-methylpiperidin-3-yl)carbamate (0.090 g, 88%) as white solid. LC-MS: m/z 644.20 [M+H]⁺.

### Step 9: 3-(4-(9-(4-((3R,5R)-5-amino-1-methylpiperidin-3-yl)phenyl)-3,9-diazaspiro[5.5]undecan-3-yl)-2-methylphenyl)piperidine-2,6-dione

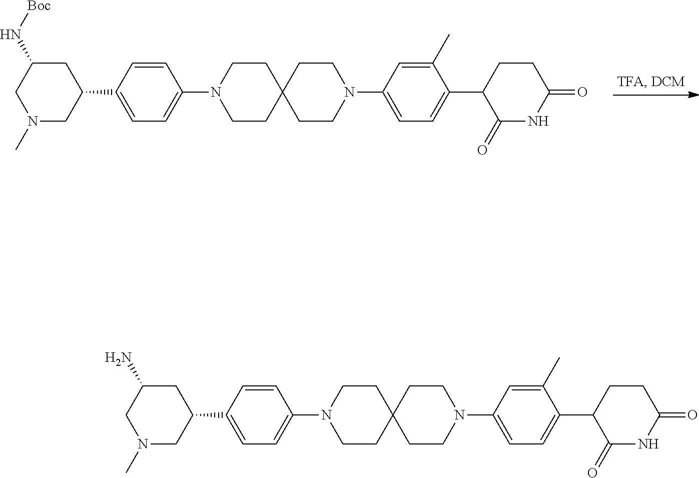 [SMILES: Cc1cc(N2CCC3(CCN(c4ccc([C@H]5C[C@@H](NC(=O)OC(C)(C)C)CN(C)C5)cc4)CC3)CC2)ccc1C1CCC(=O)NC1=O ; Cc1cc(N2CCC3(CCN(c4ccc([C@H]5C[C@@H](N)CN(C)C5)cc4)CC3)CC2)ccc1C1CCC(=O)NC1=O]

To a stirred solution of tert-butyl ((3R,5R)-5-(4-(9-(4-(2,6-dioxopiperidin-3-yl)-3-methylphenyl)-3,9-diazaspiro[5.5]undecan-3-yl)phenyl)-1-methylpiperidin-3-yl)carbamate (0.09 g, 0.13 mmol, from step 8) in DCM (5 mL), was added TFA (1 mL) at 0° C. and the reaction mixture was stirred at RT for 1 h. The progress of the reaction was monitored by TLC. After completion of reaction, the reaction mixture was concentrated under reduced pressure and then triturated with methanol and diethyl ether, respectively, to afford 3-(4-(9-(4-((3R,5R)-5-amino-1-methylpiperidin-3-yl)phenyl)-3,9-diazaspiro[5.5]undecan-3-yl)-2-methylphenyl)piperidine-2,6-dione (0.07 g, 99%, TFA salt). LC-MS: m/z 544.20 [M+H]⁺.

### Step 10: 3-(4-(9-(4-((3R,5R)-5-((5-bromo-1-methyl-6-oxo-1,6-dihydropyridazin-4-yl)amino)-1-methylpiperidin-3-yl)phenyl)-3,9-diazaspiro[5.5]undecan-3-yl)-2-methylphenyl)piperidine-2,6-dione

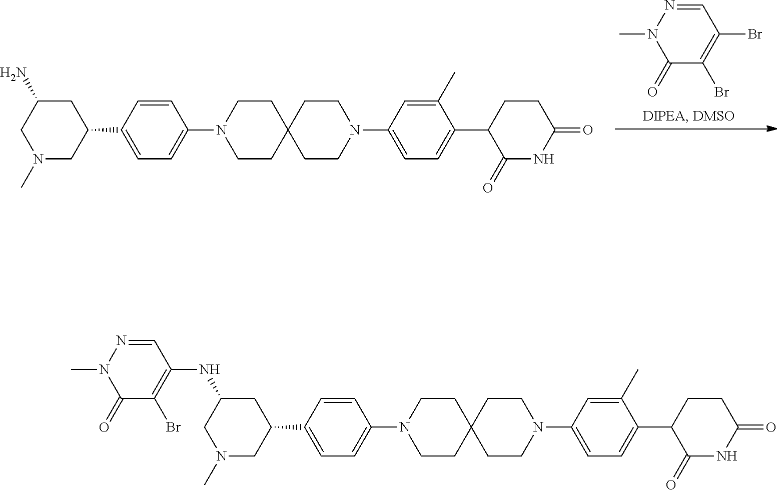

To a stirred of 3-(4-(9-(4-((3R,5R)-5-amino-1-methylpiperidin-3-yl)phenyl)-3,9-diazaspiro[5.5]undecan-3-yl)-2-methylphenyl)piperidine-2,6-dione (0.07 g, 0.10 mmol, from step 9) in DMSO (4 mL) were added N,N-diisopropylethylamine (0.06 mL, 0.32 mmol) and 4,5-dibromo-2-methyl-pyridazin-3-one (0.03 g, 0.13 mmol) and the reaction mixture was stirred at 120° C. for 2 h in a microwave. The progress of the reaction was monitored by LCMS and TLC. After completion of the reaction, the reaction mixture was filtered through a celite pad. The filtrate was diluted with cold water and extracted with ethyl acetate. The organic layer was washed with water and brine, dried over anhydrous sodium sulphate, filtered, and concentrated under reduced pressure to obtain crude compound. The crude was purified by silica gel column chromatography where the product was eluted with 7% MeOH in DCM to afford 3-(4-(9-(4-((3R,5R)-5-((5-bromo-1-methyl-6-oxo-1,6-dihydropyridazin-4-yl)amino)-1-methylpiperidin-3-yl)phenyl)-3,9-diazaspiro[5.5]undecan-3-yl)-2-methyl-phenyl)piperidine-2,6-dione (0.08 g, 10%) as a white solid. ¹H NMR (400 MHz, DMSO-d₆) δ=10.75 (s, 1H), 7.86 (s, 1H), 7.08 (d, J=8.4 Hz, 2H), 6.90-6.87 (m, 3H), 6.76 (s, 1H), 6.72 (d, J=8.4 Hz, 1H), 5.79 (d, J=9.2 Hz, 1H), 3.96-3.87 (m, 2H), 3.59 (s, 3H), 3.16-3.09 (m, 8H), 2.95-2.91 (m, 1H), 2.86-2.76 (m, 3H), 2.24-2.20 (m, 6H), 2.14-2.06 (m, 2H), 2.00-1.91 (m, 3H), 1.86-1.78 (m, 1H), 1.70-1.55 (m, 9H). LC-MS: m/z 730.30 [M+H]⁺.

Certain data for certain compounds are presented below as examples.

| | General | | |
|---|---|---|---|
| Example | Procedure | LCMS | H NMR |
| 26 | 6 | m/z | ¹H NMR (400 MHZ, DMSO-d₆) δ = 10.78 (s, 1H), 7.95 (s, 1H), |
| | | 630.20 | 7.26 (m, J = 7.6 Hz, 2H), 7.22-7.13 (m, 3H), 6.68-6.49 (m, |
| | | [M + H]⁺ | 3H), 6.15-6.03 (m, 1H), 4.01-3.86 (m, 1H), 3.78 (m, J = 4.9, |
| | | | 10.7 Hz, 1H), 3.71-3.62 (m, 2H), 3.58 (s, 3H), 3.52-3.42 (m, |
| | | | 4H), 3.28 (m, 4H), 2.97-2.87 (m, 2H), 2.82 (m, J = 2.6, 4.3 Hz, |
| | | | 1H), 2.67-2.60 (m, 1H), 2.29-2.16 (m, 4H), 2.10-2.03 (m, |
| | | | 1H), 2.02-1.96 (m, 1H), 1.91 (s, 1H), 1.86 (m, 1H), 1.67 (m, J = |
| | | | 12.0 Hz, 1H), 1.56 (m, J = 8.0 Hz, 1H) |
| 27 | 6 | m/z | ¹H NMR (400 MHZ, DMSO-d₆) δ = 10.77 (brs, 1H), 7.87 (s, 1H), |
| | | 690.0 | 7.32 (d, J = 8.1 Hz, 2H), 7.23-7.17 (m, 2H), 7.17-7.11 (m, 1H), |
| | | and | 6.84-6.78 (m, 2H), 6.61-6.57 (m, 1H), 5.83-5.77 (m, 1H), 3.98- |
| | | 692.0 | 3.92 (m, 1H), 3.78-3.72 (m, 3H), 3.59 (s, 3H), 3.56-3.50 (m, |
| | | [M + H]⁺ | 2H), 2.94-2.89 (m, 2H), 2.80-2.79 (m, 1H), 2.62-2.61 (m, 1H), |
| | | | 2.46-2.46 (m, 2H), 2.27-2.20 (m, 5H), 2.06-1.98 (m, 3H), 1.92- |
| | | | 1.84 (m, 2H), 1.74-1.64 (m, 2H), 1.02 (d, J = 6.0 Hz, 6H) |
| 28 | 6 | m/z | ¹H NMR (400 MHZ, DMSO-d₆) δ = 10.77 (brs, 1H), 7.87 (s, 1H), |
| | | 646.10 | 7.32 (d, J = 8.1 Hz, 2H), 7.23-7.17 (m, 2H), 7.17-7.11 (m, 1H), |
| | | [M + H]⁺ | 6.84-6.78 (m, 2H), 6.61-6.57 (m, 1H), 5.83-5.77 (m, 1H), 3.98- |
| | | | 3.92 (m, 1H), 3.78-3.72 (m, 3H), 3.59 (s, 3H), 3.56-3.50 (m, |
| | | | 2H), 2.94-2.89 (m, 2H), 2.80-2.79 (m, 1H), 2.62-2.61 (m, 1H), |
| | | | 2.46-2.46 (m, 2H), 2.27-2.20 (m, 5H), 2.06-1.98 (m, 3H), 1.92- |
| | | | 1.84 (m, 2H), 1.74-1.64 (m, 2H), 1.02 (d, J = 6.0 Hz, 6H) |
| 29 | 8 | m/z | ¹H NMR (400 MHz, DMSO-d₆) δ = 10.79 (s, 1H), 7.96 (s, 1H), |
| | | 733.58 | 7.35-7.30 (m, 4H), 6.77-6.70 (m, 2H), 6.59 (dd, J = 8.8, 2.0 Hz, |
| | | [M + H]⁺ | 1H), 6.11 (d, J = 8.8 Hz, 1H), 5.03 (dd, J = 7.6, 1.6 Hz, 1H), 4.29- |
| | | | 4.22 (m, 1H), 3.99-3.91 (m, 1H), 3.58 (br s, 5H), 3.31-3.28 |
| | | | (m, 1H), 3.04-2.83 (m, 7H), 2.80-2.70 (m, 1H), 2.58-2.54 (m, |
| | | | 1H), 2.25 (s, 3H), 2.12-1.88 (m, 5H), 1.74-1.38 (m, 9H). One |
| | | | aliphatic proton is merged with solvent peak. |
| 30 | 100 | m/z | ¹H NMR (400 MHZ, DMSO-d₆) δ = 10.79 (s, 1H), 7.96 (s, 1H), |
| | | 777.51 | 7.36-7.28 (m, 4H), 6.78-6.69 (m, 2H), 6.61-6.55 (m, 1H), 6.12 |
| | | and | (d, J = 9.0 Hz, 1H), 5.76 (s, 1H), 5.06-5.01 (m, 1H), 4.28-4.17 |
| | | 779.52 | (m, 1H), 4.01-3.87 (m, 1H), 3.65-3.56 (m, SH), 3.30-3.27 (m, |
| | | [M + H]⁺ | 1H), 3.04-2.90 (m, 7H), 2.79-2.73 (m, 1H), 2.60-2.56 (m, 1H), |
| | | | 2.25 (s, 3H), 2.05-1.87 (m, 5H), 1.72-1.45 (m, 9H) |
| 31 | 9 | m/z | ¹H NMR (400 MHZ, DMSO-d₆) δ = 10.50 (s, 1H), 8.14 (d, J = 5.8 |
| | | 796.0 | Hz, 1H), 7.88 (s, 1H), 7.81 (d, J = 9.5 Hz, 1H), 7.51 (d, J = 5.8 Hz, |
| | | and | 1H), 7.47-7.42 (m, 1H), 7.38-7.28 (m, 4H), 7.17-7.14 (m, 1H), |
| | | 798.0 | 5.81 (d, J = 8.9 Hz, 1H), 4.09-3.85 (m, 2H), 3.66-3.58 (m, 5H), |
| | | [M + H]⁺ | 3.48-3.39 (m, 4H), 3.31-3.24 (m, 5H), 2.97-2.92 (m, 2H), 2.86- |
| | | | 2.80 (m, 1H), 2.25 (s, 3H), 2.05-1.91 (m, 3H), 1.76-1.48 (m, |
| | | | 9H) |
| 32 | 9 | m/z | ¹H NMR (400 MHZ, DMSO-d₆) δ = 10.48 (m, 1H), 9.05 (s, 1H), |
| | | 796.0 | 8.24 (s, 1H), 7.96 (s, 1H), 7.79-7.75 (m, 1H), 7.66 (m, J = 2.3, 9.4 |
| | | and | Hz, 1H), 7.41 (d, J = 2.1 Hz, 1H), 7.37-7.29 (m, 4H), 6.10 (d, J = |
| | | 798.0 | 9.0 Hz, 1H), 4.00-3.84 (m, 2H), 3.73-3.54 (m, 5H), 3.42-3.34 |
| | | [M + H]⁺ | (m, 4H), 3.05-2.82 (m, 4H), 2.79-2.70 (m, 1H), 2.50-2.45 (m, |
| | | | 4H), 2.25 (s, 3H), 2.07-1.88 (m, 2H), 1.73-1.42 (m, 9H) |
| 33 | 9 | m/z | ¹H NMR (400 MHz, DMSO-d₆) δ = 10.50 (s, 1H), 8.14 (d, J = 5.8 |
| | | 752.0 | Hz. 1H), 7.88 (s, 1H), 7.81 (d, J = 9.5 Hz, 1H). 7.51 (d, J = 5.8 |
| | | [M + H]⁺ | Hz. 1H), 7.47-7.42 (m, 1H), 7.38-7.28 (m, 4H), 7.17-7.14 (m, |
| | | | 1H), 5.81 (d, J = 8.9 Hz, 1H), 4.09-3.85 (m, 2H), 3.66-3.58 (m, |
| | | | SH), 3.48-3.39 (m, 4H), 3.31-3.24 (m, 5H), 2.97-2.92 (m, |
| | | | 2H), 2.86-2.80 (m, 1H), 2.25 (s, 3H), 2.05-1.91 (m, 3H), 1.76- |
| | | | 1.48 (m, 9H) |
| 34 | 5 | m/z | ¹H NMR (400 MHZ, DMSO-d₆) δ = 10.92 (s, 1H), 8.39 (d, J = 5.6 |
| | | 726.0 | Hz. 1H), 8.25 (d, J = 8.9 Hz, 1H), 7.96 (s, 1H), 7.87 (s, 1H), 7.74- |
| | | and | 7.67 (m, 2H), 7.45-7.30 (m, 4H), 6.12 (d, J = 9.0 Hz, 1H), 5.03- |
| | | 728.0 | 4.96 (m, 1H), 4.75-4.62 (m, 1H), 4.02-3.67 (m, 2H), 3.58 (s, |
| | | [M + H]⁺ | 3H), 3.24-3.18 (m, 1H), 3.11-2.83 (m, 5H), 2.68-2.59 (m, |
| | | | 1H), 2.45-2.31 (m, 2H), 2.27-2.20 (m, 4H), 2.09-1.87 (m, |
| | | | 5H), 1.80-1.64 (m, 3H) |
| 35 | 5 | m/z | ¹H NMR (400 MHZ, DMSO-d₆) δ = 10.97 (s, 1H), 9.20 (s, 1H), |
| | | 662.30 | 8.35 (s, 1H), 8.04-8.01 (m, 1H), 7.97-7.93 (m, 1H), 7.81-7.75 |
| | | [M + H]⁺ | (m, 2H), 7.44-7.39 (m, 2H). 7.37-7.32 (m, 3H), 5.49 (d, J = 8.9 |
| | | | Hz. 1H), 4.71-4.57 (m, 2H), 3.87-3.77 (m, 2H), 3.52 (s, 3H), |
| | | | 3.09-2.94 (m, 4H), 2.91-2.79 (m, 3H), 2.63-2.53 (m, 2H), 2.25 |
| | | | (s, 3H), 2.13-2.03 (m, 2H), 1.95-1.87 (m, 3H), 1.85-1.81 (m, |
| | | | 3H), 1.79-1.71 (m, 2H), 1.63-1.57 (m, 1H) |
| 36 | 5 | m/z | ¹H NMR (400 MHZ, DMSO-d₆) δ = 10.92 (s, 1H), 8.39 (d, J = 5.6 |
| | | 682.27 | Hz, 1H), 8.25 (d, J = 8.9 Hz, 1H), 7.96 (s, 1H), 7.87 (s, 1H), 7.74- |
| | | [M + H]⁺ | 7.67 (m, 2H), 7.45-7.30 (m, 4H), 6.12 (d, J = 9.0 Hz, 1H), 5.03- |
| | | | 4.96 (m, 1H), 4.75-4.62 (m, 1H), 4.02-3.67 (m, 2H), 3.58 (s, |
| | | | 3H), 3.24-3.18 (m, 1H), 3.11-2.83 (m, 5H), 2.68-2.59 (m, |
| | | | 1H), 2.45-2.31 (m, 2H), 2.27-2.20 (m, 4H), 2.09-1.87 (m, |
| | | | 5H), 1.80-1.64 (m, 3H) |
| 37 | 6 | m/z | ¹H NMR (400 MHZ, DMSO-d₆) δ = 10.95 (brs, 1H), 9.01 (s, 1H), |
| | | 713.10 | 8.14 (s, 1H), 7.96 (s, 1H), 7.86-7.81 (m, 1H), 7.66-7.60 (m, 1H), |
| | | and | 7.37-7.21 (m, 5H), 6.12 (d, J = 9.0 Hz, 1H), 4.56-4.50 (m, 1H), |
| | | 715.10 | 3.99-3.90 (m, 1H), 3.58 (s, 3H), 3.52 (s, 2H), 3.01-2.76 (m, 5H), |
| | | [M + H]⁺ | 2.64-2.54 (m, 6H), 2.45-2.40 (m, 2H), 2.24 (s, 3H), 2.12-1.85 |
| | | | (m, 5H), 1.77-1.62 (m, 1H) |
| 38 | 6 | m/z | ¹H NMR (400 MHZ, DMSO-d₆) δ = 10.95 (brs, 1H), 9.01 (s, 1H), |
| | | 667.0 | 8.14 (s, 1H), 7.96 (s, 1H), 7.86-7.81 (m, 1H), 7.66-7.60 (m, 1H), |
| | | [M + H]⁺ | 7.37-7.21 (m, 5H), 6.12 (d, J = 9.0 Hz, 1H), 4.56-4.50 (m, 1H), |
| | | | 3.99-3.90 (m, 1H), 3.58 (s, 3H), 3.52 (s, 2H), 3.01-2.76 (m, 5H), |
| | | | 2.64-2.54 (m, 6H), 2.45-2.40 (m, 2H), 2.24 (s, 3H), 2.12-1.85 |
| | | | (m, 5H), 1.77-1.62 (m, 1H) |
| 39 | 6 | m/z | ¹H NMR (400 MHZ, DMSO-d₆) δ = 10.86 (s, 1H), 8.19 (d, J = 5.6 |
| | | 713.0 | Hz, 1H), 8.09 (d, J = 9.5 Hz, 1H), 7.88 (s, 1H), 7.51-7.44 (m, |
| | | and | 2H), 7.32-7.21 (m, 4H), 7.13 (d, J = 2.3 Hz, 1H), 5.80 (d, J = 9.1 |
| | | 715.0 | Hz, 1H), 4.91-4.82 (m, 1H), 4.01-3.89 (m, 1H), 3.60 (s, 3H), |
| | | [M + H]⁺ | 3.52 (s, 2H), 3.42-3.34 (m, 4H), 2.99-2.90 (m, 2H), 2.87-2.81 |
| | | | (m, 1H), 2.68-2.63 (m, 1H), 2.63-2.53 (m, 4H), 2.35-2.30 (m, |
| | | | 1H), 2.26-2.22 (m, 4H), 2.05-1.87 (m, 3H), 1.71 (q, J = 12.0 |
| | | | Hz, 1H) [IH merged in the solvent] |
| 40 | 6 | m/z | ¹H NMR (400 MHz, DMSO-d₆) δ = 10.86 (s, 1H), 8.19 (d, J = 5.6 |
| | | 669.20 | Hz, 1H), 8.09 (d, J = 9.6 Hz, 1H), 7.96 (s, 1H), 7.50-7.46 (m, 2H), |
| | | [M + H]⁺ | 7.30 (d, J = 8.4 Hz. 2H), 7.25-7.22 (d, J = 8.0 Hz, 2H), 7.13 (d, J = |
| | | | 2.0 Hz, 1H), 6.11 (d, J = 8.8 Hz, 1H), 4.86 (dd, J = 7.6, 5.6 Hz, |
| | | | 1H), 3.99-3.90 (m, 1H), 3.58 (s, 3H), 3.52 (s, 2H), 3.38 (br s, 4H), |
| | | | 2.98-2.92 (m, 2H), 2.85 (d, J = 10.8 Hz, 1H), 2.64-2.53 (m, 6H), |
| | | | 2.33-2.20 (m, 5H), 2.04-1.86 (m, 3H), 1.69 (q, J = 12.1 Hz, 1H) |
| 41 | 4 | mz | ¹H NMR (400 MHZ, DMSO-d₆) δ = 10.87 (s, 1H), 8.12 (d, J = 8.4 |
| | | 727.0 | Hz, 1H), 7.88 (s, 1H), 7.79 (d, J = 9.2 Hz, 1H), 7.60 (dd, J = 9.2, |
| | | [M + H]⁺ | 2.4 Hz, 1H), 7.44-7.35 (m, 5H), 7.22 (d, J = 2.4 Hz, 1H), 5.80 (d, |
| | | | J = 9.2 Hz, 1H), 4.18-4.12 (m, 1H), 3.99-3.94 (m, 1H). 3.85- |
| | | | 3.68 (m, 3H), 3.64-3.55 (m, 4H), 3.04-2.92 (m, 2H), 2.86 (d, J = |
| | | | 8.8 Hz, 1H), 2.64-2.57 (m, 2H), 2.41-2.36 (m, 1H), 2.26 (s, |
| | | | 3H), 2.21-2.15 (m, 1H), 2.08-1.92 (m, 4H), 1.80-1.69 (m, 1H). |
| 42 | 6 | m/z | ¹H NMR (400 MHZ, DMSO-d₆) δ = 10.86 (s, 1H), 8.10 (d, J = 8.4 |
| | | 713.52 | Hz, 1H), 7.87 (s, 1H), 7.76 (d, J = 9.2 Hz, 1H), 7.57 (dd, J = 9.2, |
| | | [M + H]⁺ | 2.4 Hz, 1H), 7.37 (d, J = 8.4 Hz, 1H), 7.29 (d, J = 8.0 Hz, 2H), |
| | | | 7.24 (d, J = 8.4 Hz, 2H), 7.16 (d, J = 2.4 Hz, 1H), 5.80 (d, J = 9.0 |
| | | | Hz, 1H), 4.14 (dd, J = 9.2, 4.8 Hz, 1H), 4.01-3.92 (m, 1H), 3.60 |
| | | | (s, 3H), 3.52 (s, 2H), 2.98-2.91 (m, 2H), 2.87-2.81 (m, 1H), |
| | | | 2.58-2.52 (m, 3H). 2.44-2.39 (m, 1H), 2.24 (s, 3H), 2.21-2.15 |
| | | | (m, 1H), 2.06-1.86 (m, 3H), 1.76-1.66 (m, 1H). Note: Seven |
| | | | aliphatic protons merged with solvent peak. |
| 43 | 10 | m/z | ¹H NMR (400 MHZ, DMSO-d₆) δ = 10.85-10.80 (m, 1H), 7.87 (s, |
| | | 697.43 | 1H, 7.33-7.15 (m, 8H), 5.81 (d, J = 9.1 Hz, 1H), 4.00-3.92 (m, |
| | | and | 1H), 3.88-3.82 (m, 1H), 3.65-3.54 (m, 5H), 3.17-3.04 (m, 2H), |
| | | 699.40 | 2.99-2.90 (m, 3H). 2.88-2.81 (m, 1H), 2.67-2.62 (m, 1H), 2.47- |
| | | [M + H]⁺ | 2.43 (m, 2H), 2.26-2.16 (m, 5H), 2.08-1.86 (m, 5H), 1.81-1.66 |
| | | | (m, 2H) |
| 44 | 10 | miz | ¹HNMR (400 MHZ, DMSO-d₆) δ = 10.82 (d, J = 2.9 Hz, 1H), 7.78 |
| | | 631.51 | (s, 1H), 7.33-7.21 (m, 6H), 7.18-7.11 (m, 2H), 5.47 (d, J = 8.8 |
| | | [M + H]⁺ | Hz, 1H), 3.90-3.77 (m, 2H), 3.59 (d, J = 8.1 Hz, 2H), 3.52 (s, 3H), |
| | | | 3.15-3.04 (m, 2H), 3.00-2.91 (m, 3H), 2.88-2.80 (m, 1H), 2.66- |
| | | | 2.60 (m, 1H), 2.44-2.38 (m, 2H), 2.24 (s, 3H), 2.20-2.15 (m, |
| | | | 2H), 2.07-2.01 (m, 3H), 1.89-1.83 (m, 5H), 1.81-1.76 (m, 1H), |
| | | | 1.63-1.51 (m, 1H) |
| 45 | 10 | m/z | ¹H NMR (400 MHZ, DMSO-d₆) δ = 10.97 (s, 1H), 9.25 (s, 1H), |
| | | 748.00 | 8.39 (s, 1H), 8.11 (s, 1H), 7.97 (d, J = 9.2 Hz, 1H), 7.88 (s, 1H), |
| | | [M + H]⁺ | 7.75 (d, J = 8.4 Hz, 1H), 7.30 (d, J = 8.0 Hz, 2H), 7.26 (d, J = 8.4 |
| | | | Hz. 2H), 5.83-5.78 (m, 1H), 4.64-4.56 (m, 1H), 4.01-3.91 |
| | | | (m, 1H), 3.68-3.62 (m, 2H), 3.59 (s, 3H), 3.19-3.13 (m, 1H), |
| | | | 3.01-2.93 (m, 4H), 2.88-2.75 (m, 2H), 2.64-2.55 (m, 1H), 2.45- |
| | | | 2.39 (m, 2H), 2.30-2.22 (m, 4H), 2.14-2.07 (m, 1H), 2.05- |
| | | | 2.00 (m, 2H), 1.95-1.87 (m, 3H), 1.77-1.67 (m, 1H) |
| 46 | 10 | m/z | ¹H NMR (400 MHZ, DMSO-d₆) δ = 10.97 (s, 1H), 9.25 (s, 1H), |
| | | 704.30 | 8.38 (s, 1H), 8.11 (s, 1H), 7.99-7.95 (m, 2H), 7.75 (d, J = 8.4 |
| | | [M + H]⁺ | Hz, 1H), 7.31 (d, .] = 8.4 Hz, 2H), 7.25 (d, J = 8.4 Hz, 2H), 6.10 |
| | | | (d, J = 8.4 Hz, 1H), 4.64-4.56 (m, 1H), 4.01-3.91 (m, 1H), |
| | | | 3.69-3.60 (m, 2H), 3.58 (s, 3H), 3.19-3.13 (m, 1H), 3.01-2.93 |
| | | | (m, 3H), 2.88-2.78 (m, 2H), 2.65-2.55 (m, 2H), 2.46-2.40 (m, |
| | | | 2H), 2.30-2.21 (m, 5H), 2.15-1.86 (m, 5H), 1,77-1.67 (m, 1H) |
| 47 | 10 | m/z | ¹H NMR (400 MHZ, DMSO-d₆) δ = 10.82 (brs, 1H), 7.96 (s, 1H), |
| | | 653.42 | 7.32-7.19 (m, 6H), 7.17-7.13 (m, 2H), 6.11 (d, J = 8.8 Hz, 1H), |
| | | [M + H]⁺ | 3.98-3.82 (m, 2H), 3.64-3.55 (m, 5H), 3.16-3.04 (m, 2H), 2.95- |
| | | | 2.91 (m, 2H), 2.87-2.81 (m, 1H), 2.68-2.60 (m, 1H), 2.42-2.31 |
| | | | (m, 2H), 2.26-2.16 (m, 5H), 2.09-1.85 (m, 6H), 1.82-1.62 (m, |
| | | | 2H) |
| 48 | 11 | m/z | ¹H NMR (400 MHZ, DMSO-d₆) δ = 11.12 (s, 1H), 7.94-7.82 (m, |
| | | 766.22 | 4H), 7.31-7.23 (m, 4H), 5.81 (d, J = 9.2 Hz, 1H), 5.15 (dd, J = |
| | | and | 12.8, 5.2 Hz, 1H), 3.98-3.92 (m, 1H), 3.68-3.59 (m, 5H), 3.55- |
| | | 768.23 | 3.42 (m, 1H), 3.18-3.09 (m, 1H), 2.99-2.82 (m, 4H), 2.64-2.57 |
| | | [M + H]⁺ | (m, 2H), 2.42-2.38 (m, 1H), 2.26-2.20 (m, 5H), 2.11-1.83 (m, |
| | | | 6H), 1.72 (q, J = 11.6 Hz, 1H). |
| 49 | | m/z | ¹H NMR (400 MHZ, DMSO-d₆) δ = 11.12 (brs, 1H), 7.96-7.80 |
| | | 722.26 | (m, 4H), 7.27 (q, J = 8.3 Hz, 4H), 5.84-5.77 (m, 1H), 5.19-5.08 |
| | | [M + H]⁺ | (m, 1H), 4.01-3.88 (m, 1H), 3.73-3.57 (m, 6H), 3.50-3.42 (m, |
| | | | 1H), 3.01-2.83 (m, 5H), 2.28-2.17 (m, 6H), 2.11-1.80 (m, 7H), |
| | | | 1.78-1.64 (m, 1H) |
| 50 | 12 | m/z | ¹H NMR (400 MHz, DMSO-d₆) δ = 10.16 (s, 1H), 7.96 (s, 1H), |
| | | 633.70 | 7.31-7.14 (m, 5H), 6.85-6.79 (m, 2H), 6.68 (d, J = 7.5 Hz, 1H), |
| | | [M + H]⁺ | 6.14-6.09 (m, 1H), 4.47-4.41 (m, 2H), 3.98-3.90 (m, 1H), 3.58 |
| | | | (s, 3H), 3.48 (s, 2H), 3.26 (t, J = 6.8 Hz, 2H), 3.12 (brs, 4H), 2.99- |
| | | | 2.80 (m, 3H), 2.63-2.53 (m, 2H), 2.48-2.28 (m, 4H), 2.24 (s, |
| | | | 3H), 2.04-1.84 (m, 3H), 1.68 (q, J = 12.0 Hz, 1H) |
| 51 | 12 | mz | ¹H NMR (400 MHz, DMSO-d₆) δ = 10.16 (s, 1H), 7.96 (s, 1H), |
| | | 677.50 | 7.31-7.14 (m, 5H), 6.85-6.79 (m, 2H), 6.68 (d, J = 7.5 Hz, 1H), |
| | | [M + H]⁺ | 6.14-6.09 (m, 1H), 4.47-4.41 (m, 2H), 3.98-3.90 (m, 1H), 3.58 |
| | | | (s, 3H), 3.48 (s, 2H), 3.26 (t, J = 6.8 Hz, 2H), 3.12 (br s, 4H), 2.99 |
| | | | -2.80 (m, 3H). 2.63-2.53 (m, 2H), 2.48-2.28 (m, 4H), 2.24 (s, |
| | | | 3H), 2.04-1.84 (m, 3H), 1.68 (q, J = 12.0 Hz, 1H) |
| 52 | 9 | m/z | ¹H NMR (400 MHZ, DMSO-d₆) δ = 10.22 (s, 1H), 7.87 (s, 1H), |
| | | 759.51 | 7.36-7.31 (m, 4H), 7.03 (d, ) = 8.6 Hz, 1H), 6.83-6.75 (m, 2H), |
| | | and | 5.80 (d, J = 9.0 Hz, 1H), 4.00-3.88 (m, 1H), 3.79-3.51 (m, 6H), |
| | | 761.45 | 3.50-3.40 (m, 1H), 3.29-3.27 (m, 1H), 3.20-3.11 (m, 4H), 3.03- |
| | | [M + H]⁺ | 2.91 (m, 2H), 2.88-2.82 (m, 1H), 2.73-2.64 (m, 2H), 2.25 (s, |
| | | | 3H), 2.11 (s, 3H), 2.08-1.90 (m, 4H), 1.72 (q, J = 11.9 Hz, 1H), |
| | | | 1.66-1.56 (m, 4H), 1.55-1.41 (m, 4H) |
| 53 | 9 | m/z | ¹H NMR (400 MHZ, DMSO-d₆) δ = 10.22 (s, 1H), 7.87 (s, 1H), |
| | | 715.10 | 7.36-7.31 (m, 4H), 7.03 (d, J = 8.6 Hz, 1H), 6.83-6.75 (m, 2H), |
| | | [M + H]⁺ | 5.80 (d, J = 9.0 Hz. 1H), 4.00-3.88 (m, 1H), 3.79-3.51 (m, 6H), |
| | | | 3.50-3.40 (m, 1H), 3.29-3.27 (m, 1H), 3.20-3.11 (m, 4H), 3.03- |
| | | | 2.91 (m, 2H), 2.88-2.82 (m, 1H), 2.73-2.64 (m, 2H), 2.25 (s, |
| | | | 3H), 2.11 (s, 3H), 2.08-1.90 (m, 4H), 1.72 (q, J = 11.9 Hz, 1H), |
| | | | 1.66-1.56 (m, 4H), 1.55-1.41 (m, 4H) |
| 54 | 9 | miz | ¹H NMR (400 MHZ, DMSO-d₆) δ = 10.35 (s, 1H), 7.87 (s, 1H), |
| | | 763.10 | 7.33 (s, 4H), 7.18 (t, J = 9.0 Hz, 1H), 6.83-6.74 (m, 2H), 5.80 (d, |
| | | [M + H]⁺ | J = 9.0 Hz, 1H), 4.00-3.92 (m, 1H), 3.63-3.59 (m, 6H), 3.34- |
| | | | 3.32 (m, 1H), 3.30-3.29 (m, 1H), 3.24-3.18 (m, 4H), 3.03-2.93 |
| | | | (m, 2H), 2.88-2.83 (m, 1H), 2.68 (t, J = 6.8 Hz, 2H), 2.45-2.41 |
| | | | (m, 1H), 2.25 (s, 3H), 2.05-1.91 (m, 3H), 1.72 (q, J = 12.0 Hz, |
| | | | IFD), 1.59 (br s, 4H), 1.54-1.43 (m, 4H) |
| 55 | 9 | m/z | ¹H NMR (400 MHZ, DMSO-d₆) δ = 10.35 (s, 1H), 7.95 (s, 1H), |
| | | 719.53 | 7.36-7.30 (m, 4H), 7.18 (t, J = 9.0 Hz, 1H), 6.83-6.74 (m, 2H), |
| | | [M + H]⁺ | 6.10 (d, J = 9.2 Hz, 1H), 4.00-3.92 (m, 1H), 3.63-3.59 (m, 6H), |
| | | | 3.24-3.18 (m, 4H), 3.03-2.91 (m, 2H), 2.88-2.83 (m, 1H), 2.68 |
| | | | (t, J = 6.8 Hz, 2H), 2.25 (s, 3H), 2.07-1.84 (m, 3H), 1.72 (q, J = |
| | | | 12.0 Hz, 1H), 1.59 (br s, 4H), 1.54-1.43 (m, 4H). Note: Three |
| | | | protons merged with solvent peak. |
| 56 | 13 | m/z | ¹H NMR (400 MHz, DMSO-d₆) δ = 10.22 (s, 1H), 7.96 (s, 1H), |
| | | 729.59 | 7.35-7.31 (m, 4H), 6.66 (s, 2H), 6.10 (d, J = 9.2 Hz, 1H), 3.97- |
| | | [M + H]⁺ | 3.91 (m, 1H), 3.66-3.61 (m, 1H), 3.58 (s, 3H), 3.50-3.46 (m, 2H), |
| | | | 3.19-3.13 (m, 4H), 3.00-2.92 (m, 3H), 2.88-2.83 (m, 1H), 2.70- |
| | | | 2.65 (m, 2H), 2.25 (s, 3H), 2.09 (s, 6H), 2.04-1.90 (m, 3H), 1.73- |
| | | | 1.64 (m, 1H), 1.60-1.55 (m, 4H), 1.52-1.37 (m, 4H) (Note: Two |
| | | | protons merged with solvent peak) |
| 57 | 14 | m/z | ¹H NMR (400 MHZ, DMSO-d₆) δ = 10.16 (brs, 1H), 7.86 (s, 1H), |
| | | 731.00 | 7.16 (t, J = 8.0 Hz, 1H), 7.09 (d, J = 8.8 Hz, 2H), 6.91-6.83 (m, |
| | | [M + H]⁺ | 4H), 6.65 (d, J = 7.5 Hz, 1H), 5.79 (d, J = 8.9 Hz, 1H), 4.44 (s, |
| | | | 2H), 3.95-3,90 (m, 1H), 3.59 (s, 3H), 3.27-3.24 (m, 4H), 3.17- |
| | | | 3.10 (m, 8H), 2.94-2.91 (m, 1H), 2.86-2.75 (m, 2H), 2.22 (s, |
| | | | 3H), 1.99-1.90 (m, 2H), 1.82 ( t, J = 10.6 Hz, 1H), 1.71-1.63 |
| | | | (m, 1H), 1.61-1.57 (m, 8H) |
| 58 | 14 | m/z | ¹H NMR (400 MHz, DMSO-d₆) δ = 10.16 (s, 1H), 7.94 (s, 1H), |
| | | b687.30 | 7.16 (t. J = 8.4 Hz, 1H), 7.08 (br d, J = 8.4 Hz, 2H), 6.90-6.83 |
| | | [M + H]⁺ | (m, 4H), 6.65 (d, J = 8.4 Hz, 1H), 6.09 (d, J = 8.8 Hz, 1H), 4.44 |
| | | | (s, 2H), 3.96-3.88 (m, 1H), 3.58 (s, 3H), 3.29-3.25 (m, 2H), |
| | | | 3.18-3.10 (m, 8H), 2.94-2.89 (m, 1H), 2.86-2.77 (m, 2H), 2.22 |
| | | | (s, 3H), 1.98-1.87 (m, 2H), 1.85-1.75 (m, 1H), 1.67-1.55 (m, |
| | | | 9H). Note: Two protons merged with solvent peak. |
| 59 | | m/z | ¹H NMR (400 MHZ, DMSO-d₆) δ = 10.83 (s, 1H), 8.68 (d, J = 8.0 |
| | | 788.0 | Hz, 1H), 8.31 (d, J = 2.8 Hz, 1H), 7.87 (s, 1H), 7.84 (d, J = 8.8 |
| | | [M + H]⁺ | Hz, 1H), 7.40 (dd, J = 2.8, 8.8 Hz, 1H), 7.33 (s, 4H), 5.80 (d, J = |
| | | | 8.8 Hz, 1H), 4.76-4.69 (m, 1H), 4.00-3.92 (m, 1H), 3.65-3.55 |
| | | | (m, 5H), 3.43-3.35 (m, 4H), 3.05-2.92 (m, 2H), 2.87-2.74 (m, |
| | | | 2H), 2.55-2.50 (m, 3H), 2.25 (s, 3H), 2.21-2.12 (m, 1H), 2.08- |
| | | | 1.89 (m, 4H), 1.75-1.67 (m, 1H), 1.62 (br s, 4H), 1.57-1.41 (m, |
| | | | 4H) |
| 60 | | m/z | ¹H NMR (400 MHZ, DMSO-d₆) δ = 10.83 (s, 1H), 8.69 (d, J = 8.25 |
| | | 744.10 | Hz, 1H), 8.31 (d, J = 2.75 Hz, 1H), 7.96 (s, 1H), 7.84 (d, J = 8.88 |
| | | [M + H]⁺ | Hz, 1H), 7.40 (dd, J = 9.01, 2.88 Hz, 1H), 7.33 (d, J = 1.63 Hz, |
| | | | 4H), 6.10 (d, J = 9.01 Hz, 1H), 4.77-4.70 (m, 1H), 4.00-3.89 (m, |
| | | | 1H), 3.66-3.58 (m, 5H), 3.40-3.35 (m, 4H), 3.04-2.93 (m, 2H), |
| | | | 2.88-2.75 (m, 2H), 2.60-2.52 (m, 2H), 2.26-2.24 (m, 3H), 2.19- |
| | | | 2.15 (m, 1H), 2.04-1.91 (m, 4H), 1.74-1.67 (m, 2H), 1.62 (br |
| | | | s, 4H), 1.56-1.44 (m, 4H) |
| 61 | 7 | m/z | ¹H NMR (400 MHZ, DMSO-d₆) δ = 10.82 (s, 1H), 7.98 (t, J = 7.19 |
| | | 805.57 | Hz, 1H), 7.87 (s, 1H), 7.62 (t, J = 9.07 Hz, 1H), 7.33 (s, 4H), 6.83- |
| | | and | 6.72 (m, 2H), 5.80 (d, J = 8.88 Hz, 1H), 4.77-4.68 (m, 1H), 4.01- |
| | | 807.59 | 3.91 (m, 1H), 3.59 (s, 6H), 3.19-3.15 (m, 1H), 3.03-2.92 (m, |
| | | [M + H]⁺ | 3H), 2.87-2.76 (m, 3H), 2.25 (s, 3H), 2.10-1.95 (m, 6H), 1.77- |
| | | | 1.70 (m, 1H), 1.61-1.4 (m, 9H). Note: One proton merged with |
| | | | solvent peak. |
| 62 | 7 | m/z. | ¹H NMR (400 MHZ, DMSO-d₆) δ = 10.82 (s, 1H), 7.98 (t, J = 7.19 |
| | | 761.20 | Hz, 1H). 7.87 (s, 1H), 7.62 (t, J = 9.07 Hz, 1H), 7.33 (s, 4H), 6.83- |
| | | [M + H]⁺ | 6.72 (m, 2H), 5.80 (d, J = 8.88 Hz, 1H), 4.77-4.68 (m, 1H), 4.01- |
| | | | 3.91 (m, 1H), 3.59 (s, 6H), 3.19-3.15 (m, 1H), 3.03-2.92 (m, |
| | | | 3H), 2.87-2.76 (m, 3H), 2.25 (s, 3H), 2.10-1.95 (m, 6H), 1.77- |
| | | | 1.70 (m, 1H), 1.61-1.40 (m, 9H) Note: One proton merged with |
| | | | solvent peak. |
| 63 | 15 | m/z | ¹H NMR (400 MHZ, DMSO-d₆) δ = 8.95 (s, 1H), 8.58 ( t, J = 5.9 |
| | | 1042.20, | Hz. 1H), 7.87 (s, 1H), 7.75 (d, J = 9.6 Hz, 1H), 7.45-7.36 (m, |
| | | 1044.20 | 4H), 7.32 (s, 4H), 5.80 (d, J = 9.0 Hz, 1H), 5.13 (d, J = 3.4 Hz, 1H), |
| | | [M + H]⁺ | 4.52-4.22 (m, 6H), 4.01-3.91 (m, 1H), 3.68-3.59 (m, 6H), 3.29- |
| | | | 3.24 (m, 4H), 3.07-2.81 (m, 7H), 2.63-2.53 (m, 5H), 2.45-2.41 |
| | | | (m, 3H), 2.25 (s, 3H), 2.08-1.87 (m, 5H), 1.83-1.62 (m, 3H), 1.41- |
| | | | 1.28 (m, 2H), 0.93 (s, 9H) |
| 64 | 15 | m/z | ¹H NMR (400 MHZ, DMSO-d₆) δ = 8.95 (s, 1H), 8.58 (t, J = 6.0 |
| | | 1042.10 | Hz, 1H), 7.87 (s, 1H), 7.76 (d, J = 9.8 Hz, 1H), 7.44-7.36 (m, 4H), |
| | | and | 7.33-7.30 (m, 4H), 5.80 (d, J = 9.0 Hz, 1H), 5.13 (d, J = 3.4 Hz, |
| | | 1044.10 | 1H), 4.49 (d, J = 9.6 Hz, 1H), 4.46-4.33 (m, 3H), 4.29-4.24 (m, |
| | | [M + H]⁺ | 1H), 4.01-3.90 (m, 1H), 3.68-3.64 (m, 1H), 3.62-3.57 (m, 4H), |
| | | | 3.55-3.49 (m, 2H), 3.04-2.82 (m, 8H), 2.43 (s, 3H), 2.25 (s, 3H), |
| | | | 2.21-1.85 (m, 8H), 1.82-1.67 (m, 3H), 1.50-1.36 (m, 2H), 0.94 |
| | | | (s, 9H). Note: Five aliphatic protons merged with solvent peak. |
| 65 | 15 | m/z | ¹H NMR (400 MHZ, DMSO-d₆) δ = 8.98 (s, 1H), 8.54 (t. J = 6.0 |
| | | 1027.15 | Hz, 1H), 7.87 (s, 1H), 7.73 (d, J = 9.3 Hz, 1H), 7.43-7.37 (m, 4H), |
| | | and | 7.32 (s, 4H), 5.80 (d, J = 9.0 Hz, 1H), 5.12 (d, J = 3.5 Hz, 1H), |
| | | 1029.10 | 4.53-4.33 (m, 5H), 4.22 ( dd, J = 15.8, 5.4 Hz, 1H), 3.99-3.92 (m, |
| | | [M + H]⁺ | 1H), 3.69-3.59 (m, 6H), 3.04-2.81 (m, 8H), 2.45 (s, 3H), 2.25 (s, |
| | | | 3H), 2.15-1.88 (m, 8H), 1.78-1.65 (m, 4H), 1.58-1.46 (m, 3H), |
| | | | 1.45-1.30 (m, 2H), 0.93 (s, 9H) |
| 66 | 15 | m/z | ¹H NMR (400 MHZ, DMSO-d₆) δ = 8.98 (s, 1H), 8.54 (t, J = 6.0 |
| | | 958.10 | Hz. 1H), 7.90-7.86 (m, 2H), 7.44-7.36 (m, 4H), 7.34-7.28 (m, |
| | | and | 4H), 5.79 (d, J = 9.0 Hz, 1H), 5.09 (d, J = 3.3 Hz. 1H), 4.54 (d, J = |
| | | 960.10 | 9.4 Hz. 1H), 4.47-4.40 (m, 2H), 4.35 (brs, 1H), 4.25-4.18 (m, |
| | | [M + H]⁺ | 1H), 4.00-3.92 (m, 1H), 3.69-3.53 (m, 6H), 3.03-2.92 (m, 3H), |
| | | | 2.87-2.82 (m, 2H), 2.44 (s, 3H), 2.28-2.19 (m, 4H), 2.16-2.10 |
| | | | (m, 1H), 2.06-1.87 (m, 7H), 1.76-1.52 (m, 3H), 1.16-1.02 (m, |
| | | | 2H), 0.93 (s, 9H) |
| 67 | 15 | m/z | ¹H NMR (400 MHZ, DMSO-d₆) δ = 8.98 (s, 1H), 8.54 (t, J = 6.0 |
| | | 1012.20 | Hz. 1H), 7.87 (s, 1H), 7.70 (d, J = 9.3 Hz, 1H), 7.43-7.37 (m, 4H), |
| | | and | 7.32 (s, 4H), 5.80 (d, J = 9.0 Hz, 1H), 5.11 ( d, J = 3.1 Hz, 1H), |
| | | 1014.20 | 4.51 (d, J = 8.0 Hz, 1H), 4.46-4.39 (m, 2H), 4.34 (brs, 1H), 4.25- |
| | | [M + H]⁺ | 4.18 (m, 1H), 4.00-3.92 (m, 1H), 3.69-3.49 (m, 7H), 3.29-3.20 |
| | | | (m, 2H), 3.03-2.92 (m, 2H), 2.85 (d, J = 10.5 Hz, 1H), 2.44 (s, |
| | | | 3H), 2.38-2.29 (m, 1H), 2.25 (s, 3H), 2.06-1.99 (m, 3H), 1.92- |
| | | | 1.84 (m, 2H), 1.77-1.70 (m, 3H), 1.60-1.42 (m, 6H), 1.35-1.23 |
| | | | (m, 2H), 1.14-1.03 (m, 2H), 0.93 (s, 9H) |
| 68 | 15 | m/z | ¹H NMR (400 MHZ, DMSO-d₆) δ = 8.93 (s, 1H), 8.59 (t, J = 6.1 |
| | | 1027.00 | Hz. 1H), 7.88 (s, 1H), 7.79 (d, J = 9.8 Hz, 1H), 7.47-7.36 (m, 4H), |
| | | and | 7.35-7.28 (m, 4H), 5.80 (d, J = 9.0 Hz, 1H), 5.13 ( d, J = 2.3 Hz, |
| | | 1029.20 | 1H), 4.50 (d, J = 9.8 Hz. 1H), 4.45-4.40 (m, 1H), 4.38-4.34 (m, |
| | | [M + H]⁺ | 2H), 4.29-4.23 (m, 1H), 4.00-3.91 (m, 1H), 3.68-3.64 (m, 1H), |
| | | | 3.62-3.53 (m, 5H), 3.04-2.92 (m, 4H), 2.89-2.83 (m, 1H), 2.56- |
| | | | 2.53 (m, 1H), 2.45-2.37 (m, 8H). 2.25 (s, 3H), 2.08-1.86 (m, 5H), |
| | | | 1.72 (q, J = 12.0 Hz, 1H), 1.60-1.27 (m, 9H), 0.94 (s, 9H) |
| 69 | 15 | m/z | ¹H NMR (400 MHZ, DMSO-d₆) δ = 8.98 (s, 1H), 8.54 (t, J = 6.1 |
| | | 1041.20 | Hz, 1H), 7.87 (s, 1H), 7.75 (d, 1H), 7.44-7.36 (m, 4H), 7.35-7.28 |
| | | and | (m, 4H), 5.80 (d, J = 9.1 Hz, 1H), 5.11 (d, J = 3.4 Hz, 1H), 4.51 (d, |
| | | 1043.30 | J = 9.4 Hz, 1H), 4.45-4.39 (m, 2H), 4.35 (brs, 1H), 4.25-4.19 (m, |
| | | [M + H]⁺ | 1H), 3.98-3.90 (m, 1H), 3.68-3.58 (m, 6H), 3.02-2.93 (m, 3H), |
| | | | 2.87-2.80 (m, 4H), 2.44 (s, 3H), 2.36-2.32 (m, 1H), 2.25 (s, 3H), |
| | | | 2.14-2.06 (m, 2H), 2.05-1.89 (m, 6H), 1.84-1.63 (m, 7H), 1.59- |
| | | | 1.46 (m, 3H), 1.08-0.90 (m, 11H) |
| 70 | 15 | m/z | ¹H NMR (400 MHz, DMSO-d₆) δ = 8.98 (s, 1H), 8.57 (t, J = 6.0 |
| | | 958.90 | Hz, 1H), 7.87 (s, 1H), 7.77 (d, J = 9.6 Hz, 1H), 7.46-7.37 (m, 4H), |
| | | and | 7.33 (s, 4H), 5.79 (d, J = 9.0 Hz, 1H), 5.12 (d, J = 3.4 Hz, 1H), 4.51 |
| | | 960.90 | (d, J = 9.6 Hz, 1H), 4.45-4.40 (m, 1H), 4.38-3.34 (m, 2H), 4.30- |
| | | [M + H]⁺ | 4.24 (m, 1H), 3.99-3.92 (m, 1H). 3.68-3.59 (m, 6H), 3.45-3.41 |
| | | | (m, 1H), 3.18-3.04 (m, 2H), 3.00-2.93 (m, 2H), 2.86-2.82 (m, |
| | | | 1H), 2.44 (s, 3H), 2.25 (s, 3H), 2.07-1.85 (m, 5H), 1.72 (q, J = |
| | | | 11.6 Hz, 1H), 0.94 (s, 9H). Note: Four aliphatic protons merged |
| | | | with solvent peak. |
| 71 | 15 | m/z | ¹H NMR (400 MHZ, DMSO-d₆) δ = 8.97 (s, 1H), 8.58 (t. J = 6.0 |
| | | 1154.30 | Hz, 1H), 8.43 (t, J = 5.6 Hz, 1H), 8.15 (s, 1H), 7.88 (s, 1H), 7.80 |
| | | and | (d, J = 8.0 Hz, 2H), 7.46-7.35 (m, SH), 7.34 (d, J = 8.4 Hz, 2H), |
| | | 1156.30 | 5.81 (d, J = 9.1 Hz, 1H), 5.17-5.11 (m, 1H), 4.56 (d, J = 9.5 Hz, |
| | | [M + H]⁺ | 1H), 4.47-4.35 (m, 3H), 4.27-4.23 (m, 1H), 3.96 (s, 3H), 3.69- |
| | | | 3.65 (m, 1H), 3.62-3.59 (m, 6H), 3.57-3.50 (m, 12H), 3.48-3.45 |
| | | | (m, 8H), 3.42-3.39 (m, 2H), 3.05-2.92 (m, 2H), 2.86-2.82 (m, |
| | | | 1H), 2.44 (s, 3H), 2.25 (s, 3H), 2.08-1.88 (m, 5H), 1.77-1.68 (m, |
| | | | 1H), 0.95-0.92 (m, 9H). Compound present as formate salt. |
| 72 | 15 | m/z | ¹H NMR (400 MHZ, DMSO-d₆) δ = 8.98 (s, 1H), 8.54 (t, J = 5.6 |
| | | 960.00 | Hz, 1H), 8.35 (t, J = 5.6 Hz, 1H), 7.88 (s, 1H), 7.83 (d, J = 9.4 Hz, |
| | | and | 1H), 7.78 (d, J = 8.4 Hz, 2H), 7.43-7.37 (m, 4H), 7.33 (d, J = 8.4 |
| | | 962.00 | Hz, 2H), 5.81 (d, ) = 9.2 Hz, 1H), 5.11 (d, J = 3.6 Hz, 1H), 4.54 (d, |
| | | [M + H]⁺ | J = 9.4 Hz, 1H), 4.46-4.39 (m, 2H), 4.35 (brs, 1H), 4.21 (dd, J = |
| | | | 5.4, 16.0 Hz, 1H), 4.00-3.92 (m, 1H), 3.68-3.63 (m, 2H), 3.59 (s, |
| | | | 3H), 3.26-3.20 (m, 2H), 3.04-2.92 (m, 2H), 2.83 (dd, J = 2.9, |
| | | | 10.7 Hz, 1H), 2.44 (s, 3H), 2.28-2.22 (m, 4H), 2.16-2.09 (m, 1H), |
| | | | 2.06-1.87 (m, 5H), 1.72 (q, J = 12.0 Hz, 1H), 1.54-1.43 (m, 4H), |
| | | | 1.31-122 (m, 4H), 0.93 (s, 9H) |
| 73 | 15 | m/z | ¹H NMR (400 MHZ, DMSO-d₆) δ = 8.97 (s, 1H), 8.58 (t, J = 5.6 |
| | | 1066.10 | Hz, 1H), 8.42 (t, J = 5.6 Hz, 1H), 7.88 (s, 1H), 7.79 (d, J = 8.4 Hz, |
| | | and | 2H), 7.46-7.35 (m, 5H), 7.33 (d, J = 8.0 Hz, 2H), 5.80 (d, J = 9.1 |
| | | 1068.10 | Hz. 1H), 5.14 (d, J = 3.4 Hz, 1H), 4.58-4.55 (m, 1H), 4.47-4.33 |
| | | [M+H]+ | (m, 3H), 4.29-4.22 (m, 1H), 3.99-3.90 (m, 3H), 3.69-3.64 (m, |
| | | | 2H), 3.61-3.58 (m, 5H), 3.56-3.49 (m, 12 H), 3.42-3.38 (m, |
| | | | 2H), 3.05-2.92 (m, 2H), 2.84 ( dd, J = 2.7, 10.8 Hz, 1H), 2.44 (s, |
| | | | 3H), 2.24 (s, 3H), 2.08-1.87 (m, 5H), 1.77-1.66 (m, 1H), 0.95- |
| | | | 0.91 (m, 9H) |
| 74 | 15 | m/z | ¹H NMR (400 MHZ, DMSO-d₆) δ = 8.97 (s, 1H), 8.58 (t, J = 6.1 |
| | | 1022.00 | Hz, 1H), 8.42 (t, J = 5.5 Hz, 1H), 7.88 (s, 1H), 7.79 (d, J = 8.3 Hz, |
| | | and | 2H), 7.43-7.38 (m, 5H), 7.33 (d, J = 8.3 Hz, 2H), 5.80 (d, J = 9.2 |
| | | 1024.10 | Hz. 1H), 5.14 ((d, J = 3.2 Hz, 1H), 4.56 (d, J = 9.6 Hz, 1H), 4.47- |
| | | [M + H]⁺ | 4.33 (m, 3H), 4.29-4.21 (m, 1H), 4.01-3.91 (m, 3H), 3.70-3.48 |
| | | | (m, 15H), 3.42-3.39 (m, 3H), 3.04-2.93 (m, 2H), 2.87-2.81 (m, |
| | | | 1H), 2.44 (s, 3H), 2.24 (s, 3H), 2.09-1.87 (m, 4H). 1.77-1.67 (m, |
| | | | 1H), 0.93 (s, 9H) |
| 75 | 15 | m/z | ¹H NMR (400 MHZ, DMSO-d₆) δ = 8.98 (s, 1H), 8.54 (t, J = 6.0 |
| | | 988.30 | Hz. 1H), 8.35 (t. J = 5.6 Hz, 1H), 7.88 (s, 1H), 7.84-7.75 (m, 3H), |
| | | and | 7.43-7.30 (m, 6H), 5.81 (d, J = 9.1 Hz, 1H), 5.11 (d, J = 3.5 Hz, |
| | | 990.30 | 1H), 4.57-4.51 (m, 1H), 4.47-4.40 (m, 2H), 4.35 (brs, 1H), 4.25- |
| | | [M + H]⁺ | 4.19 (m, 1H), 4.01-3.91 (m, 1H), 3.68-3.62 (m, 2H), 3.59 (s, |
| | | | 3H), 3.24-3.20 (m, 2H), 3.03-2.95 (m, 2H), 2.86-2.81 (m, 1H), |
| | | | 2.44 (s, 3H), 2.28-2.21 (s, 3H), 2.14-2.08 (m, 1H), 2.04-1.97 (m, |
| | | | 3H), 1.94-1.88 (m, 2H), 1.77-1.68 (m, 1H), 1.53-1.41 (m, 4H), |
| | | | 1.26 (brs, 8H), 0.93 (s, 9H) |
| 76 | 15 | m/z | ¹H NMR (400 MHZ, DMSO-d₆) δ = 8.98 (s, 1H), 8.54 (t, J = 5.6 |
| | | 946.66 | Hz. 1H), 8.35 (t, J = 5.6 Hz, 1H), 7.88 (s, 1H), 7.84-7.76 (m, 3H), |
| | | and | 7.43-7.30 (m, 6H), 5.81 (d, J = 9.1 Hz, 1H), 5.11 (d, J = 3.5 Hz, |
| | | 948.67 | 1H), 4.53 (d, J = 9.4 Hz, 1H), 4.46-4.40 (m, 2H), 4.35 (brs, 1H), |
| | | [M + H]⁺ | 4.21 (dd, J = 5.4, 15.8 Hz, 1H), 4.00-3.92 (m, 1H), 3.67-3.59 (m, |
| | | | 5H), 3.25-3.20 (m, 2H), 3.04-2.93 (m, 2H), 2.83 (dd, J = 2.6, |
| | | | 10.6 Hz, 1H), 2.44 (s, 3H), 2.28-2.02 (m, 4H), 2.17-2.05 (m, 1H), |
| | | | 2.06-1.98 (m, 3H), 1.96-1.88 (m, 2H), 1.72 (q, ) = 12.0 Hz, 1H), |
| | | | 1.57-1.47 (m, 4H), 1.27 (td, J = 7.5, 14.7 Hz, 2H), 0.94-0.90 (m, |
| | | | 9H) |
| 77 | 15 | m/z | ¹H NMR (400 MHZ, DMSO-d₆) δ = 8.98 (s, 1H), 8.54 (t, J = 5.6 |
| | | 930.0 | Hz. 1H), 8.38 (t, J = 5.6 Hz, 1H), 7.90-7.75 (m, 4H), 7.43-7.32 |
| | | and | (m, 6H), 5.81 (d, J = 9.1 Hz, 1H), 5.11 (d, J = 3.5 Hz, 1H), 4.54 (d, |
| | | 932.20 | J = 9.3 Hz, 1H), 4.46-4.41 (m, 2H), 4.35-4.32 (m, 1H), 4.21 (dd, |
| | | [M + H]⁺ | J = 5.4, 15.8 Hz, 1H), 4.00-3.92 (m, 1H), 3.69-3.63 (m, 2H), |
| | | | 3.59 (s, 3H), 3.28-3.20 (m, 2H), 3.04-2.93 (m, 2H), 2.85-2.81 (m, |
| | | | 1H), 2.44 (s, 3H), 2.28-2.23 (m, 4H), 2.18-2.12 (m, 1H), 2.07- |
| | | | 1.96 (m, 3H), 1.93-1.86 (m, 2H), 1.72 (q, J = 12.2 Hz, 1H), 1.56- |
| | | | 1.44 (m, 4H), 0.93 (s, 9H) |
| 78 | 15 | m/z | ¹H NMR (400 MHZ, DMSO-d₆) δ = 8.96 (s, 1H), 8.51 (t, J = 6.0 |
| | | 978.00 | Hz, 1H), 8.43 (t, J = 5.6 Hz, 1H), 7.87 (s, 1H), 7.78 (d, J = 8.4 |
| | | and | Hz, 2H), 7.44-7.40 (m, 1H), 7.38 (s, 4H), 7.33 (d, J = 8.3 Hz, |
| | | 980.00 | 2H), 5.80 (d, J = 9.0 Hz, 1H). 5.15 (brs, 1H), 4.57 (d, J = 9.5 Hz. |
| | | [M + H]⁺ | 1H), 4.45 (t, J = 8.3 Hz, 1H), 4.39-4.34 (m, 2H), 4.28-4.22 (m, |
| | | | 1H), 4.01-3.91 (m, 3H), 3.67-3.55 (m, 10H), 3.45-3.41 (m, |
| | | | 2H), 3.03-2.93 (m, 3H), 2.85-2.81 (m, 1H), 2.46-2.43 (m, |
| | | | 3H), 2.24 (s, 3H), 2.10-1.98 (m, 3H), 1.95-1.88 (m, 2H), 1.71 |
| | | | (d, J = 11.9 Hz, 1H), 0.93 (s, 9H). |

### Example B1. Biological Assay of KAT2 Degradation

### Cell Culture

MOLM-13 cells or NCI-H1048 cells were used for Western Blot analysis of KAT2A degradation. KAT2B degradation can be measured in a similar manner with NCI-H1048 cells.

MOLM-13 cells were maintained at 37° C. in 5% CO₂ and 90% humidity in RPMI 1640 supplemented with 20% fetal bovine serum (FBS) and 1× penicillin/streptomycin. Cells were plated in full media at 1-2 million cells/2 mL in a 6-well tissue culture treated plate with or without compounds for 24 h at 37° C. in 5% CO₂.

H1048 cells were maintained at 37° C. in 5% CO₂ and 90% humidity in HITES medium with 5% fetal bovine serum (FBS). HITES medium was DMEM: 12 with the following additions: 0.005 mg/ml Insulin, 0.01 mg/ml Transferrin, 30 nM Sodium selenite, 10 nM Hydrocortisone, 10 nM beta-estradiol, and extra 2 mM L-glutamine (for final concentration of 4.5 mM). Cells were plated in full media at 1 million cells/2 mL in a 6-well tissue culture treated plate with or without compounds for 24 h at 37° C. in 5% CO₂.

### Western Blot Analysis

MOLM-13 cells or NCI-H1048 cells were washed with PBS, then lysed with RIPA buffer containing 1×protease and phosphatase inhibitors (SantaCruz Biotechnologies Cat #sc-24948A). Protein was isolated by centrifugation at 12,000×g for 10 minutes at 4° C. and supernatant was collected. Protein was quantified via Bradford assay. Samples were diluted to 4 μg lysate in 16 μL RIPA buffer and then 4 μL of Jess Sample Buffer. Samples were heated at 95° C. for 5 minutes. Samples were loaded onto 12-230 kDa Jess™ Separation Module, 8×25 capillary cartridges (SM-W004, Biotechne). Antibodies loaded were: Primary antibody for KAT2A (GCN5L2 (C26A10) Rabbit mAb (3305, CST)) and for KAT2B (PCAF (C14G9)) Rabbit mAb (3378, CST) at 1:1000 and Anti-Rabbit Detection Module for Jess™ (Cat No #DM-001, Biotechne). Signal was detected by chemiluminescence.

DC₅₀ values were determined from percent reduction of protein as compared to the DMSO control. Results of the Western Blot analysis are presented in Table 3. Compounds denoted as “A” had a DC₅₀<1 nM; compounds denoted as “B” bad a DC₅₀≥1 nM and ≤30 nM; and compounds denoted as “C” had a DC₅₀>30 nM.

TABLE 3

| | KAT2A | | KAT2B |
|---|---|---|---|
| | MOLM-13 | NCI-H1048 | NCI-H1048 |
| Compound No. | DC₅₀ | DC₅₀ | DC₅₀ |
| 1 | B | | |
| 2 | B | | |
| 3 | A | | |
| 4 | C | | |
| 5 | B | | |
| 6 | A | | |
| 7 | B | | |
| 8 | B | | |
| 9 | A | | |
| 10 | B | | |
| 11 | C | | |
| 12 | B | | |
| 13 | B | | |
| 14 | A | | |
| 15 | B | | |
| 17 | | B | B |
| 19 | B | | |
| 22 | A | A | A |
| 25 | A | | |
| 26 | B | | |
| 27 | A | | |
| 28 | A | A | A |
| 29 | A | | |
| 30 | B | | |
| 31 | B | | |
| 32 | B | | |
| 33 | A | | |
| 34 | B | | |
| 35 | A | | |
| 36 | B | | |
| 37 | A | | |
| 38 | B | | |
| 39 | A | A | B |
| 40 | A | | |
| 41 | A | | |
| 42 | B | | |
| 43 | C | | |
| 44 | B | | |
| 45 | A | | |
| 46 | B | | |
| 47 | C | | |
| 48 | A | | |
| 49 | A | | |
| 50 | A | | |
| 51 | C | | |
| 52 | A | | |
| 53 | B | | |
| 54 | A | | |
| 55 | A | | |
| 56 | C | | |
| 57 | B | | |
| 58 | B | | |
| 59 | B | | |
| 60 | B | | |
| 61 | B | | |
| 62 | B | | |
| 63 | A | | |
| 64 | B | | |
| 65 | C | | |
| 66 | A | | |
| 67 | A | A | A |
| 68 | A | | |
| 69 | A | | |
| 70 | A | | |
| 71 | A | | |
| 72 | A | A | A |
| 73 | B | | |
| 74 | A | | |
| 75 | B | | |
| 76 | B | | |
| 77 | B | | |
| 78 | C | | |
| 79 | B | | |
| 80 | A | | |
| 81 | B | | |
| 82 | A | | |
| 83 | B | | |
| 84 | A | A | B |
| 85 | C | | |
| 86 | A | A | B |
| 87 | B | | |
| 88 | A | | |
| 89 | A | | |
| 90 | A | A | A |
| 91 | C | | |
| 92 | B | | |
| 93 | A | | |
| 94 | C | | |
| 95 | A | | |
| 96 | A | | |
| 97 | A | | |
| 98 | B | | |
| 99 | A | | |
| 100 | A | | |
| 101 | B | | |
| 102 | C | | |
| 103 | A | | |
| 104 | B | | |
| 105 | B | | |
| 106 | A | | |
| 107 | A | | |
| 108 | A | | |
| 109 | A | | |
| 110 | B | B | B |
| 111 | A | | |
| 112 | A | | |
| 113 | A | | |
| 114 | A | A | C |
| 115 | A | | |
| 116 | A | | |
| 117 | A | | |
| 118 | A | | |
| 119 | B | | |
| 120 | B | A | C |
| 121 | B | | |
| 122 | B | | |
| 123 | A | A | C |
| 124 | A | A | B |
| 125 | A | A | C |
| 126 | A | A | B |
| 127 | B | | |
| 128 | B | | |
| 129 | B | | |
| 130 | B | | |
| 131 | B | | |
| 132 | A | | |
| 133 | B | | |
| 134 | A | | |
| 135 | A | | |
| 136 | C | | |
| 137 | B | | |
| 138 | A | | |
| 139 | A | | |
| 140 | C | | |
| 141 | A | | |
| 142 | B | | |
| 143 | A | | |
| 144 | A | | |
| 145 | A | A | A |
| 146 | A | A | A |
| 147 | B | | |
| 148 | B | | |
| 149 | B | | |
| 150 | A | | |
| 151 | B | | |
| 152 | B | A | B |
| 153 | B | | |
| 154 | A | | |
| 155 | A | A | A |
| 156 | A | | |
| 157 | A | A | A |
| 158 | A | | |
| 159 | B | | |
| 160 | C | | |
| 161 | C | | |
| 162 | A | A | B |
| 163 | A | | |
| 164 | B | | |
| 165 | A | | |
| 166 | A | | |
| 167 | A | A | A |
| 168 | A | A | B |
| 169 | A | | |
| 170 | A | | |
| 171 | C | | |
| 172 | B | | |
| 173 | B | | |
| 174 | A | | |
| 175 | A | A | B |
| 176 | A | | |
| 177 | C | | |
| 178 | B | | |
| 179 | A | | |
| 180 | A | | |
| 181 | B | | |
| 182 | A | | |
| 183 | B | | |
| 184 | B | A | C |
| 185 | A | | |
| 186 | A | A | A |
| 187 | A | A | B |
| 188 | A | A | C |
| 189 | | B | C |
| 190 | | B | C |
| 191 | | B | C |
| 192 | | A | C |
| 193 | | B | C |
| 194 | | B | C |
| 195 | | B | C |
| 196 | | A | A |
| 197 | | B | C |
| 198 | | B | C |
| 199 | | C | C |
| 200 | | B | C |
| 201 | | A | B |
| 202 | | B | C |
| 203 | | A | B |
| 204 | | C | C |
| 205 | | C | C |
| 206 | | C | C |
| 207 | | A | C |
| 208 | | A | A |
| 209 | | A | A |
| 210 | | B | A |
| 211 | | A | A |
| 212 | | B | C |
| 213 | | A | B |
| 214 | | B | C |
| 215 | | A | A |
| 216 | | A | A |
| 217 | | B | C |
| 218 | | A | B |
| 219 | | A | A |
| 220 | | A | B |
| 221 | | C | C |
| 222 | | C | C |
| 223 | | A | A |
| 224 | | C | C |
| 225 | | A | B |
| 226 | | A | B |
| 227 | | A | B |
| 228 | | A | A |
| 229 | | A | A |
| 230 | | B | B |
| 231 | | A | B |
| 232 | | A | B |
| 233 | | A | B |
| 234 | | A | A |
| 235 | | A | A |
| 236 | | A | B |
| 237 | | A | B |
| 238 | | A | B |
| 239 | | A | A |
| 240 | | A | A |
| 241 | | A | B |
| 242 | | B | C |
| 243 | | B | C |
| 244 | | B | C |
| 245 | | A | B |
| 246 | | A | A |
| 247 | | A | B |
| 248 | | A | B |
| 249 | | A | B |
| 250 | | A | A |
| 251 | | A | B |
| 252 | | A | B |
| 253 | | A | |
| 254 | | A | B |
| 255 | | A | A |
| 256 | | A | B |
| 257 | | A | B |
| 258 | | A | C |
| 259 | | A | B |
| 260 | | A | B |
| 261 | | A | A |
| 262 | | A | B |
| 263 | | A | A |
| 264 | | A | A |
| 265 | | A | A |
| 266 | | A | A |
| 267 | | A | A |
| 268 | | A | C |
| 269 | | B | C |
| 270 | | C | C |
| 271 | | A | A |
| 272 | | A | C |
| 273 | | A | A |
| 274 | | A | A |
| 275 | | B | B |
| 276 | | A | A |
| 277 | | C | C |
| 278 | | B | C |
| 279 | | A | C |
| 280 | | B | |
| 281 | | B | |
| 282 | | A | |
| 283 | | A | A |
| 284 | | A | C |
| 285 | | A | C |
| 286 | | A | B |
| 287 | | A | B |
| 288 | | A | A |
| 289 | | A | C |
| 290 | | B | C |
| 291 | | A | B |
| 292 | | A | B |
| 293 | | A | B |
| 294 | | A | A |
| 295 | | A | B |
| 296 | | A | A |
| 297 | | A | A |
| 298 | | A | C |
| 299 | | A | B |
| 300 | | A | A |
| 301 | | A | A |
| 302 | | B | B |
| 303 | | A | B |
| 304 | | A | B |
| 305 | | B | C |
| 306 | | B | C |
| 307 | | A | B |
| 308 | | A | B |
| 309 | | A | C |
| 310 | | A | C |
| 311 | | B | C |
| 312 | | A | C |
| 313 | | A | A |
| 314 | | A | B |
| 315 | | A | B |
| 316 | | A | A |
| 317 | | A | A |
| 318 | | A | B |
| 319 | | A | B |
| 320 | | A | B |
| 321 | | A | A |
| 322 | | A | A |
| 323 | | A | A |
| 324 | | A | A |
| 325 | | A | A |
| 326 | | A | A |
| 327 | | A | B |
| 328 | | A | A |
| 329 | | A | A |
| 330 | | A | C |
| 331 | | A | C |
| 332 | | A | C |
| 333 | | A | C |
| 334 | | A | B |
| 335 | | A | C |
| 336 | | A | C |
| 337 | | A | C |
| 338 | | A | A |
| 339 | | A | A |
| 340 | | A | A |
| 341 | | A | B |
| 342 | | A | B |
| 343 | | C | C |
| 344 | | C | C |
| 345 | | C | |
| 346 | | C | C |
| 347 | | B | C |
| 348 | | A | B |
| 349 | | A | C |
| 350 | | R | C |
| 351 | | B | C |
| 352 | | B | C |
| 353 | | A | C |
| 354 | | B | C |
| 355 | | B | C |
| 356 | | B | C |
| 357 | | B | C |
| 358 | | C | C |
| 359 | | C | C |
| 360 | | C | C |
| 361 | | A | B |
| 362 | | A | B |
| 363 | | A | B |
| 364 | | A | C |
| 365 | | A | A |
| 366 | | A | B |
| 367 | | A | C |
| 368 | | B | C |
| 2-44 | B | A | A |
| 2-52 | | A | A |
| 2-69 | | A | A |
| 369 | | B | C |
| 370 | | B | |
| 371 | | B | |
| 372 | | A | B |
| 373 | | A | B |
| 374 | | A | C |
| 375 | | B | |
| 376 | | C | C |
| 377 | | A | C |
| 378 | | B | C |
| 379 | | A | C |
| 380 | | A | A |
| 381 | | B | A |
| 382 | | A | A |
| 383 | | A | B |
| 384 | | A | A |
| 385 | | B | B |
| 386 | | B | C |
| 387 | | A | C |
| 388 | | A | A |
| 389 | | A | A |
| 390 | | B | C |
| 391 | | C | A |
| 392 | | B | |
| 393 | | C | |
| 394 | | | |
| 395 | | A | A |
| 396 | | A | A |
| 397 | | A | A |
| 398 | | A | A |
| 399 | | C | C |
| 400 | | B | |
| 401 | | A | C |
| 402 | | A | A |
| 403 | | A | B |
| 404 | | A | B |
| 405 | | A | B |
| 406 | | C | |
| 407 | | | |
| 408 | | C | C |
| 409 | | B | C |
| 410 | | | C |
| 411 | | C | C |
| 412 | | | |
| 413 | | | |
| 414 | | | |
| 415 | | C | C |
| 416 | | C | C |
| 417 | | C | C |
| 418 | | C | C |
| 419 | | C | C |
| 420 | | C | C |
| 421 | | C | C |
| 422 | | C | C |
| 423 | | C | C |
| 424 | | C | C |
| 425 | | A | A |
| 426 | | B | |
| 427 | | A | A |
| 428 | | C | |
| 429 | | A | B |
| 430 | | A | C |
| 431 | | B | |
| 432 | | A | C |
| 433 | | A | B |
| 434 | | A | A |
| 435 | | C | C |
| 436 | | A | B |

In some embodiments, assessment was performed in LASCPC-01 cells, and reduction of KAT2A and KAT2B levels, including in some cases selectivity for one over the other, was confirmed. In some embodiments, DC₅₀ and/or DC₉₀ were assessed. In some embodiments, DC₅₀ for various compounds was about or no more than about 1 nM. In some embodiments, DC₉₀ for various compounds was about or no more than about 1 nM. In some embodiments, selectivity for the same polypeptide by a compound was observed across multiple types of cells (e.g., when assessed using DC₅₀ and/or DC₉₀). In some embodiments, certain compounds can provide improved properties and/or activities (e.g., potency). Data from certain assessments for certain compounds are presented below as examples (NT: not tested). It has been confirmed that various compounds described herein, including multiple compounds in Table 1, can be deemed to provide further improved properties and/or activities compared to the compound(s) in the table below for certain contexts, aspects, and/or uses.

| | | DC₅₀/DC₉₀/ | Compound | Compound |
|---|---|---|---|---|
| Cell Line | Polypeptide | GI₅₀ (nM) | Ref-1 | 22 |
| MOLM-13 | KAT2A | DC₅₀ | 2.2 | 0.11 |
| | | DC₉₀ | 51 | 0.6 |
| | KAT2B | DC₅₀ | 0.075 | 0.43 |
| | | DC₉₀ | 1.8 | 3.6 |
| | Growth | GI₅₀ | 60 | 1,2 |
| NCI-H1048 | KAT2A | DC₅₀ | 1.5 | 0.01 |
| | | DC₉₀ | 16 | 0.29 |
| | KAT2B | DC₅₀ | 0.1 | 0.04 |
| | | DC₉₀ | 1 | 1.44 |
| | Growth | GI₅₀ | 32 | 1.5 |
| LASCPC-01 | KAT2A | DC₅₀ | NT | 0.06 |
| | | DC₉₀ | NT | 0.67 |
| | KAT2B | DC₅₀ | NT | 0.16 |
| | | DC₉₀ | NT | 0.42 |
| | Growth | GI₅₀ | NT | 5 |

In some embodiments, provided technologies provide fast reduction of KAT2A and/or KAT2B levels, e.g., at about or no later than after about 1, 2, 3, 4, 5, 6, 7, 8, 9 or 10 hours. In some embodiments, reduction was observed at about or no more than about 4 hours after administration to cells. In some embodiments, near complete reduction was observed at about or no more than about 4 hours after administration to cells at certain compound concentrations. In some embodiments, reduction lasts for about or at least about 1, 2, 3, 4, 5, 6, 7, 8, 9, 10, 11, 12, 13, 14, 15, 16, 17, 18, 19, 20, 24, 25, 30, 36, 40, 45, or 48 hours. In some embodiments, reduction was observed at about 24 hours after administration to cells. Various technologies can be utilized to assess polypeptide reduction in accordance with the present disclosure, e.g., western blot, mass spectrometry, etc. In some embodiments, it was confirmed by mass spectrometry proteomics analysis that technologies can provide selective reduction of KAT2A and/or KAT2B levels compared to other polypeptides. In some embodiments, a provided compound provided selectivity for KAT2A over KAT2B. In some embodiments, a provided compound provided selectivity for KAT2B over KAT2A.

Various compounds were also assessed in in vitro and/or in vivo assays, confirming that provided compounds can inhibit cell proliferation and are useful for treating various diseases, disorders, or conditions including various cancers.

### Example B2. Provided Technologies can Inhibit Cell Growth

Among other things, provided technologies are useful for treating various diseases, disorders, or conditions. For example, in some embodiments, provided technologies are useful for treating cancer as confirmed in the present Example. In some embodiments, provided technologies can inhibit cell growth. In some embodiments, provided technologies can inhibit cell proliferation. In some embodiments, the present disclosure provides technologies for assessing various technologies, e.g., compounds, compositions, or methods. Among other things, the present Example provides technologies, e.g., cell growth inhibition assay, for assessing various technologies. In some embodiments, provided technologies are assessed as described below to demonstrate or confirm various properties, activities, advantages, benefits, etc. that they can provide. In some embodiments, the following cell growth inhibition methods are utilized to assess provided technologies.

AML: AML cell line MOLM-13 cells were cultured in RPMI+15% FBS and were plated at 500 cells/well in 100 uL media in a U-bottom 96-well plate. Compounds were dilute to 3×final in full media and 50 uL were added to corresponding wells. Compound and media were replenished on days 2 and 5. On day 7, cells were transferred to a 96-well black bottom plate and CyQuant was performed per manufacturer's instructions.

SCLC: SCLC cell lines NCI-H1048 and NCI-H1876 were cultured in DMEM F12 media with 5% FBS, 1% Penicillin/Streptomycin and HITES supplement (Hydrocortisone, Insulin, Transferrin, Estradiol and Sodium Selenite). NCI-H1048 cells were plated at 10,000 cells/well of a 24-well plate and NCI-H1876 cells were plated at 20,000 cells/well of a 96-well plate. Compound and media were replenished every two days out to day 11 and 10 respectively. Cells were then trypsinized and counted using either hemocytometer or an automated Countess cell counter.

Neuroendocrine prostate cancer: Neuroendocrine prostate cancer line LASCPC-01 cells were cultured in DMEM F12 media with 5% FBS, 1% Penicillin/Streptomycin and HITES supplement (Hydrocortisone, Insulin, Transferrin, Estradiol and Sodium Selenite). Cells were plated at 1,425 cells/well of a 96 well plate. Compound and media were replenished every two days and at day 7 cell viability was assayed using Cell Titer Glo following manufacturer's instructions.

Certain data are presented below as examples.

| | MOLM13 | H1048 | H1876 | LASCPC |
|---|---|---|---|---|
| Compound | GI50 | GI50 | GI50 | GI50 |
| No. | (nM) | (nM) | (nM) | (nM) |
| 22 | 2.9 | 1 | 1 | 5.3 |
| 40 | 7.8 | | | |
| 2-15 | 5.2 | 2 | | |
| 39 | 5.6 | | | |
| 84 | 1.1 | | | |
| 86 | 1.7 | 11 | | |
| 90 | 0.9 | 1.5 | | |
| 145 | 1 | | | |
| 146 | 5.9 | | | |
| 157 | 0.9 | | | |
| 167 | 4.8 | | | |
| 219 | 5.9 | | | |
| 223 | 4.4 | | | |
| 228 | 0.9 | | | |
| 229 | 0.7 | | | |
| 362 | 33.3 | | | |
| 363 | 5.7 | | | |
| 231 | 7.3 | | | |
| 232 | 5.2 | | | |
| 233 | 5.5 | | | |
| 238 | 20 | 60 | | |
| 240 | 3.9 | | | |
| 2-52 | 0.4 | | | |
| 242 | >100 | | | |
| 243 | >100 | | | |
| 208 | 0.3 | | | |
| 250 | 3.7 | | | |
| 437 | 0.3 | | | |

As confirmed herein, provided compounds can degrade KAT2A and/or KAT2B (see, e.g., Example B1), and can inhibit cell growth. Without the intention to be limited by any particular theory, Applicant notes that among other things, the present disclosure identifies association of KAT2A and/or KAT2B with dysregulated cell states and various diseases, disorders, or conditions, and in various cases, degradation of KAT2A and/or KAT2B are associated with inhibition of cell growth and proliferation. Efficacy of provided compounds, e.g., for inhibit cancer cell growth, has also been confirmed in animals. Certain data are presented in Example B3.

### Example B3. Provided Technologies can Inhibit Tumor Growth In Vivo

Among other things, provided technologies are useful for treating various diseases, disorders, or conditions. For example, in some embodiments, provided technologies are useful for treating cancer as confirmed in the present Example. In some embodiments, the present disclosure provides technologies for assessing various technologies, e.g., compounds, compositions, or methods. Among other things, the present Example provides technologies, e.g., xenograft model, for assessing various technologies. In some embodiments, provided technologies are assessed in tumor xenograft model. In some embodiments, provided technologies are assessed in NCI-H1048 human lung cancer xenograft model. In some embodiments, provided technologies are assessed as described below to demonstrate or confirm various properties, activities, advantages, benefits, etc. (e.g., low toxicity, high efficacy, flexibility with respect to formulation, administration, doses, dosing regimen, etc.) that they can provide.

As described herein, various provided technologies are assessed in cancer xenograft models. Procedures related to animal handling, care, and treatment are performed according to guidelines approved by relevant institutional animal care and use committee following the guidance of the Association for Assessment and Accreditation of Laboratory Animal Care (AAALAC). In assessment described below, unless indicated otherwise, animals are BALB/c nude mice, female, with ages of 6 to 8 weeks and body weight of 18 to 22 grams. Those skilled in the art appreciate other animals may be utilized. Groups and treatments start when the mean tumor volume reaches about 200 mm³. Based on tumor volumes, mice are randomly assigned to groups for various treatment. In an assessment, there are 10 animals per group for vehicle control and each compound treatment. A number of doses were assessed including 30 mg/kg. Assessed compounds were administered orally or via the intraperitoneal route. Dosing can and were conducted at various frequencies over several weeks (e.g. BID, QD, Q2D, Q3D, Q3D, QSD, Q6D or QW). Certain information of a vehicle control and a compound treatment is presented below as examples:

| | | Number | Dose | Vol | | | |
|---|---|---|---|---|---|---|---|
| Group | Treatment | of Mice | (mg/kg) | (mL/kg) | Route | Formulation | Regimen |
| 1 | Vehicle | 10 | — | 10 | p.o. | 20% HPBCD | qd × 28 days |
| | Control | | | | | in water | |
| 2 | Compound 22 | 10 | 30 | 10 | i.p. | 20% HPBCD | qw × 4 times |
| | | | | | | in water | |

Vehicle control was 20% HPBCD in water. In some embodiments, HPBCD promotes solubility. In some embodiments, various provided compounds can be formulated without using HPBCD. In some embodiments, various compounds, e.g., those comprising basic nitrogen in linkers, can be formulated in water with pH adjustment, e.g., water+2 mM citrate buffer pH 3.5. In some embodiments, formulations are to provide compounds in solutions with suitable concentrations for administration, and vehicle control is blank formulation.

Cell culture: NCI-H1048 tumor cells are maintained in vitro as monolayer culture in HITES Medium supplemented with 5% fetal bovine serum at 37° C. in an atmosphere of 5% CO₂ in air. The tumor cells are routinely sub-cultured weekly by trypsin-EDTA treatment, not to exceed 4-5 passages. The cells growing in an exponential growth phase are harvested and counted for tumor inoculation.

Method for Tumor Inoculation and Randomization: Each mouse is inoculated subcutaneously on the right flank with NCI-H1048 tumor cells (3×10⁶) in 100 μL of Medium/Matrigel mixtures (1:1) for tumor development. Treatment is started when mean tumor size reaches range of about 200 mm³. Mice are randomized into multiple groups. Compounds are administered to the mice according to various routes. e.g., p.o., i.p., doses and regimens.

Measurement Parameters: For routine monitoring, animals are monitored for tumor growth, behavior (e.g., mobility, food and water consumption), body weight (BW), eye/hair matting and other effects. Mortality, abnormal clinical signs, etc. are recorded.

Body Weight: Body weight is typically weighed at staging and once more right before tumor collection. Body weight change, expressed in %, can be calculated using the following formula: BW change (%)=BW_{Day x}/BW_{Day 0}×100, wherein BW_{Day x} is BW on a given day, and BW_{Day0} is BW on Day 0 (initiation of treatment).

Tumor Measurements: Measurement of tumor size can be conducted with a caliper and tumor volume (mm³) can be estimated using the formula: TV=a×b²/2, wherein “a” and “b” are the long and short diameters of a tumor, respectively. TVs can be utilized for calculation of tumor growth inhibition (TGI, which can be utilized as an indicator of antitumor effectiveness) value using a formula: TGI=(1−T/C)×100%, wherein “T” and “C” are mean relative volumes (% tumor growth) of tumors in a treated group and a control group, respectively. In some cases, assessment is terminated when the mean tumor volume exceeds about 2000 mm³ or severe body weight loss occurs. Tumor volume is typically measured at staging and once more right before tumor collection.

Sample Collection: At various time points, e.g., at about 5, 7, 10, 14, 15, 20, 21, 25, or 28 days, or post about 1, 2, 3, 4, 5, 7, 10, 14, 15, 20, 21, 25, 28 doses, samples can be collected for analysis. For example, blood, liver, spleen, tumor and other samples can be collected at various timepoints including post the last dose for assessing pharmacokinetics, pharmacodynamics, toxicity, tolerability, efficacy, etc. Those skilled in the art appreciate that various technologies can be utilized to analyze samples, e.g., plasma PK, whole blood CBC, western blot (e.g., KAT2A, KAT2B), qPCR, FFPE, RNA-Seq (e.g., tumor single cell RNA-Seq), H&E (e.g., with scanning), etc.

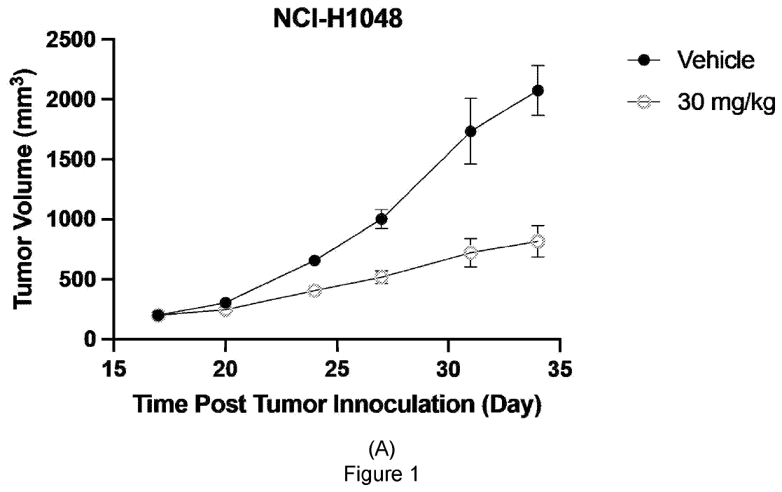

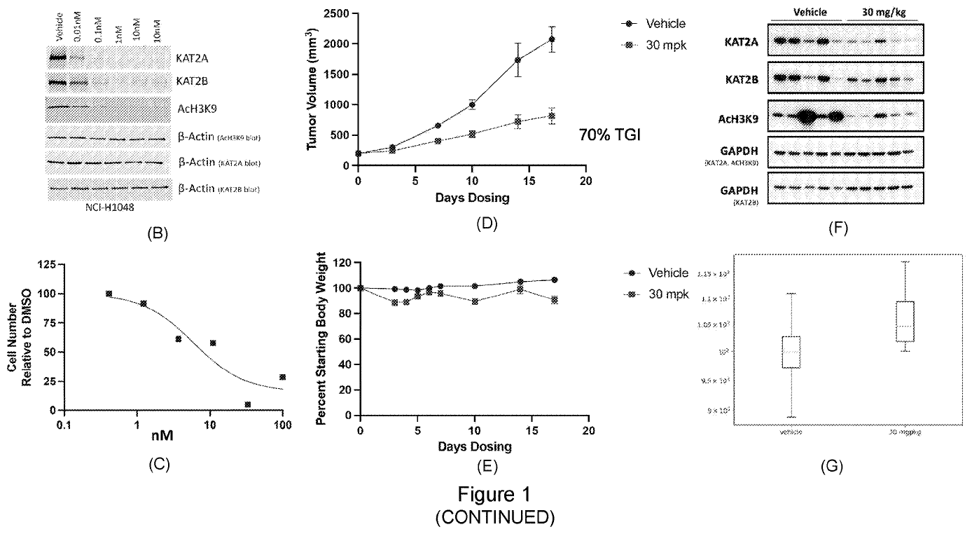

Tumor growth inhibitions at tolerated doses according to various administration routes and/or regimen were observed for assessed compounds and are expected for additional compounds, e.g., those that can degrade KAT2A and or KAT2B, e.g., when assessed in MOLM-13 and/or NCI-H1048 cells (see, e.g., Example B1), and/or can inhibit cell growth when assessed in vitro (see, e.g., Example B2). Certain data for compound 22 are presented in FIG. 1 as examples, confirming that provided compounds can not only degrade KAT2A/KAT2B and inhibit cell growth in vitro, but their activities extend to tumor growth inhibition in vivo.

### Example B4. Provided Technologies can Inhibit Tumor Growth In Vivo

Among other things, provided technologies are useful for treating various diseases, disorders, or conditions. For example, in some embodiments, provided technologies are useful for treating cancer as confirmed in the present Example. In some embodiments, the present disclosure provides technologies for assessing various technologies, e.g., compounds, compositions, or methods. Among other things, the present Example provides technologies, e.g., xenograft model, for assessing various technologies. In some embodiments, provided technologies are assessed in tumor xenograft model. In some embodiments, provided technologies are assessed in NCI-H1048 human lung cancer xenograft model. In some embodiments, provided technologies are assessed as described below to demonstrate or confirm various properties, activities, advantages, benefits, etc. (e.g., low toxicity, high efficacy, flexibility with respect to formulation, administration, doses, dosing regimen, etc.) that they can provide.

### Example B5. Provided Technologies can Modulate Cell States, Induce Differentiation and Inhibit Cell Growth

Among other things, provided technologies are useful for treating various diseases, disorders, or conditions including various cancers (e.g., acute myeloid leukemia, small cell lung cancer, neuroendocrine prostate cancer, etc.). In some embodiments, as confirmed herein provided technologies can modulate cancer cell states, induce differentiation and/or inhibit cancer cell growth.

In some embodiments, an assessment is performed using cells. In some embodiments, cells were treated with various compounds before assessing KAT2A and/or KAT2B levels, histone (e.g., H3K9) acetylation levels, cell states, growth, etc.

Various technologies can be utilized for assessment in accordance with the present disclosure. For example, in some embodiments, polypeptide levels are assessed using western blot. A useful protocol is described below as an example.

Western Blot: In some assessments, MOLM13, NCI-H1048, LASCPC-01 and NEPC organoid cells were treated with compounds at certain concentrations for certain periods of time, and cell pellets were lysed by adding 1-part 4×laemmli Sample buffer (e.g., Bio-Rad Catalog #1610747) along with Mercaptoethanol (e.g., Bio-Rad Catalog #1610710) according to manufacturer's instructions, and 1-part RIPA buffer with 2×protease and phosphatase inhibitor. The samples were sonicated 30% amplitude, 35 seconds on and 35 seconds off for 1 cycle and boiled at 95° C. for 5 minutes. 30-35 μL of samples were loaded on a 4-15% Criterion™ TGX Stain-Free™ Protein Gel (Bio-rad, 5678084). In some assessments, NCI-H1048 flash frozen tumor pieces were resuspended in RIPA buffer and homogenized on TissueLyser II with steel beads at 30 Hz for 3 minutes. Samples were then centrifuged at 12.00 rpm at 4° C. for 15 minutes. BCA protein determination was performed and samples normalized for 1.95 ug/uL. 19.5 uL were loaded and run on similar gels as cell pellets. Proteins were transferred in a nitrocellulose membrane (e.g., Trans-Blot Turbo Midi 0.2 μm Nitrocellulose Transfer Packs, Bio-Rad, 1704159) using the turbo transfer system. The membranes were blocked in TBS-Tween20 (TBST) containing 5% milk for 1 hour. The membranes were then incubated at 4° C. overnight with primary antibodies diluted in TBST containing 5% BSA. The following day, membranes were washed 3× for 10 minutes each, then incubated in anti-rabbit HRP secondary (Peroxidase AffiniPure Goat Anti-Rabbit IgG (H+L)-1:10,000) (Jackson Immunoresearch, 111-035-003) in TBS-Tween20 containing 3% milk at RT for 1 hour. Following incubation, membranes were washed 4× for 10 minutes each, and developed using SuperSignal West Femto Maximum Sensitivity Substrate (34095, Thermo Fisher). Various antibodies can be utilized for western blot. In some assessments, primary antibodies utilized were GCN5L2 (C26A10) Rabbit mAb (Catalog #3305, CST) 1:1000, Acetyl-Histone H3 (Lys9) (C5B11) Rabbit mAb (Catalog #9649, CST) 1:1000, PCAF (C14G9) Rabbit mAb (Catalog #3378S) 1:1000, and β-Actin (13E5) Rabbit mAb (Catalog #970S, CST) 1:10,000.

In some assessments, for cell growth inhibition, cells, e.g., MOLM13, NCI-H1048, and LASCPC-01 cells, were counted using a hemocytometer or automated cell counting in triplicates. In some assessments, proliferation in NEPC organoids as measured with Cell Titer Glo reagent (Promega). Growth inhibition was determined relative to vehicle control.

In some assessments, flow cytometry was utilized to assess levels of cells expressing certain polypeptides. For example, for flow cytometry for CD86 in MOLM-13, MOLM13 cells were stained with Live/Dead solution (Fixable Live/Dead NIR 1:100 in PBS) at RT, in the dark, for 20 min and then washed once with cell staining solution. The cells were then blocked with Human Fc Blocker solution at RT for 8 mins. Then they were labeled with CD86-APC R700 (565149, BD) (2.5 uL/test) on ice for 30 min in the dark. Following incubation, cells were washed once with cell staining solution. Flow cytometric acquisition was done on BD Celesta flow cytometer (BD Biosciences) and analyses were performed using Flow-Jo software.

In some embodiments, GSEA signatures are utilized for assessing cell states, differentiation, etc. In some assessments, differentiation GSEA signatures, e.g., from RNA-Seq was performed. A useful protocol is described herein as an example. In some assessments, LASCPC-01 cells, NEPC organoid cell pellets, or NCI-H1048 tumor pieces were flash frozen upon harvest and stored at −80° C. RNA was isolated, mRNA library was prepared (poly A enrichment) and sequencing was run. Reads were aligned to reference v38 using Kallis to generate gene count matrices for each sample. The gene count matrix was the input to the ML pipelines that generate normal atlas scores and cell state classification. It was also input to differential expression analysis using Edger in R. The output of differential expression was input to gene set enrichment analysis (GSEA) to define gene sets that were most shifted by treatment. For defining specific epithelial differentiation endpoints to generate the solid tumor differentiation box plots, cell state gene sets resulting from GSEA are consolidated by cell state to define a consensus set of genes that define epithelial differentiation. Other suitable technologies may also utilized in accordance with the present disclosure.

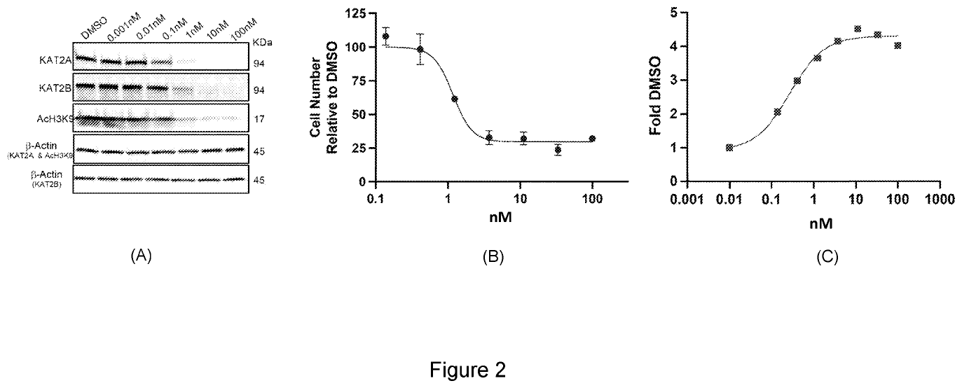

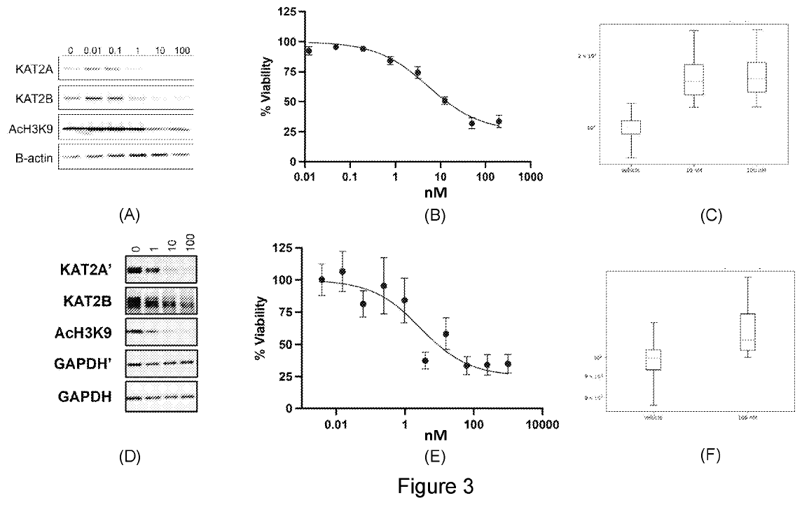

As confirmed in FIG. 1, provided compounds, e.g., compound 22, can potently reduce KAT2A and KAT2B levels, reduce histone acetylation (e.g., AcH3K9) levels, inhibit cell growth in vitro and in vivo (see also Example B3), and induce differentiation (e.g., epithelial, neural, and/or microglial) when assessed in small cell lung cancer cells, e.g., NCI-H1048 cells. Activities of provided compounds were also confirmed toward other cancers. For example, as confirmed in FIG. 2, provided compounds, e.g., compound 22, can potently reduce KAT2A and KAT2B levels, reduce histone acetylation (e.g., AcH3K9) levels, inhibit cell growth, and induce differentiation (e.g., monocytic differentiation as assessed using CD86) when assessed in acute myeloid leukemia cells such as MOLM-13 cells. As another example, as confirmed in FIG. 3, provided compounds, e.g., compound 22, can potently reduce KAT2A and KAT2B levels, reduce histone acetylation (e.g., AcH3K9) levels, inhibit cell growth in vitro and in vivo (see also Example B3), and induce differentiation (e.g., epithelial, neural, and/or microglial) when assessed in neuroendocrine prostate cancer cells, e.g., LASCPC-01 cells. Various other compounds were also active, and multiple compounds demonstrated properties and/or activities that may be deemed improved or better compared to compound 22 for certain contexts, aspects, and/or uses.

1. A compound selected from: 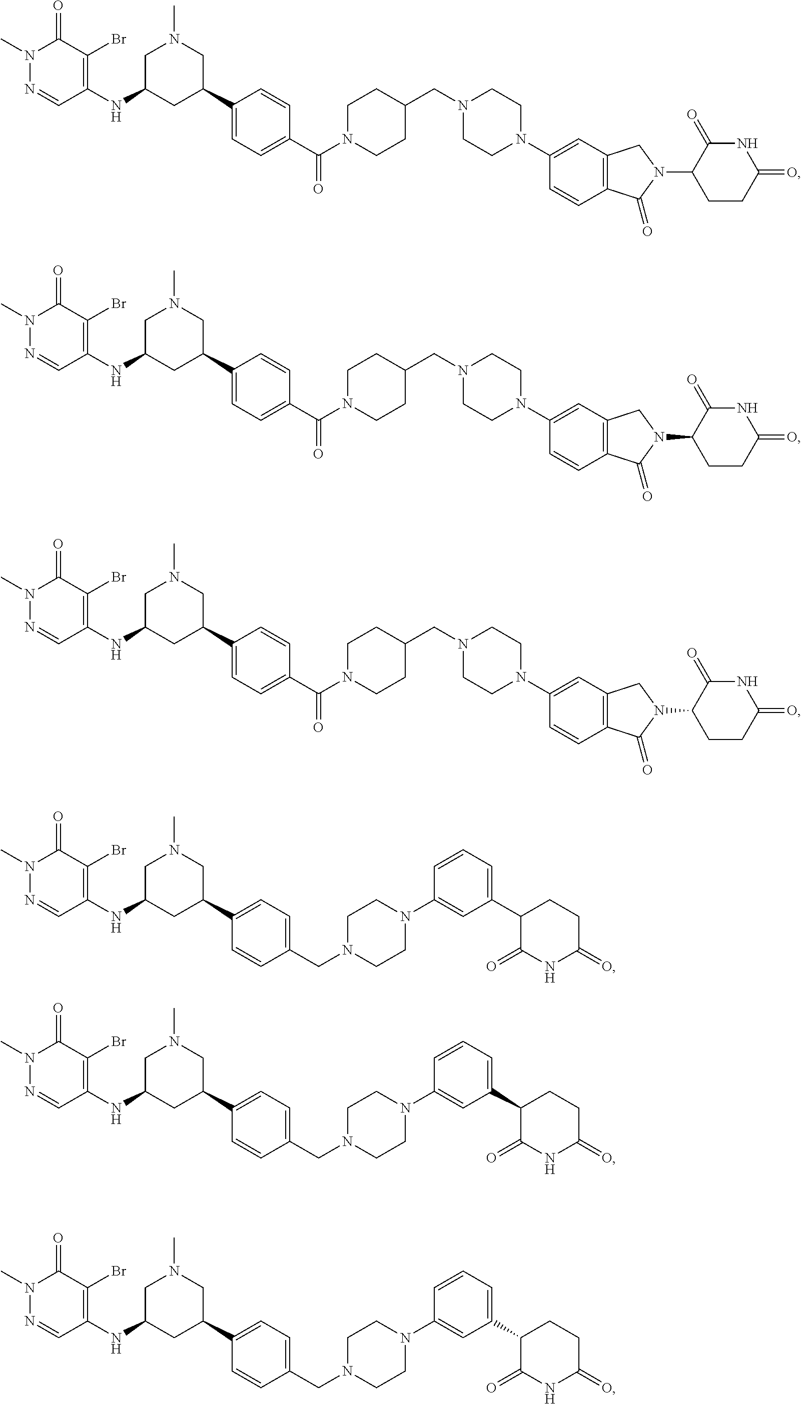 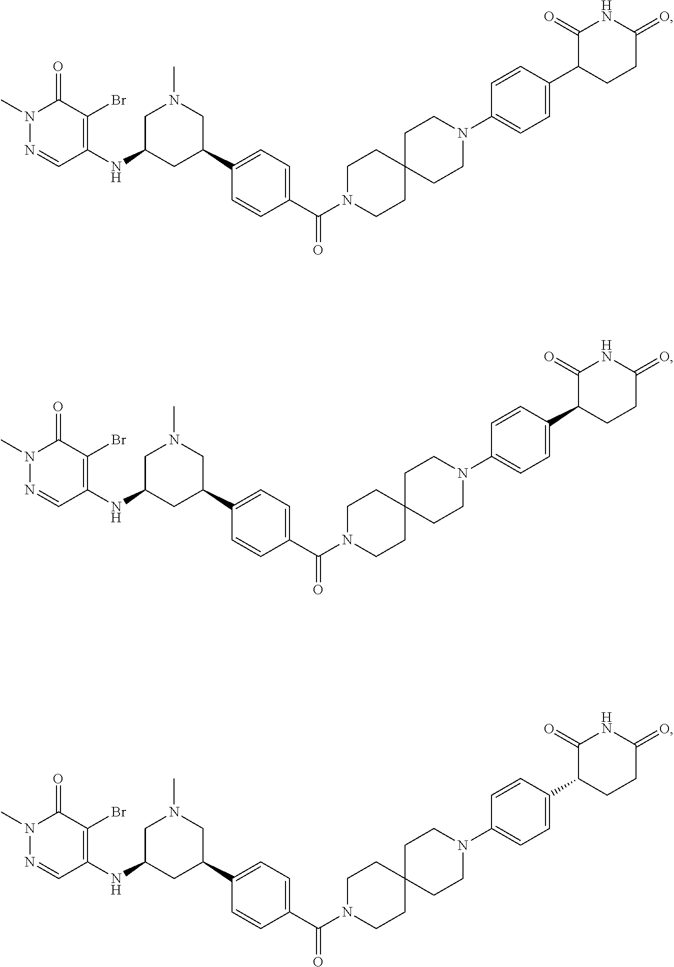 [SMILES: CN1C[C@H](Nc2cnn(C)c(=O)c2Br)C[C@H](c2ccc(C(=O)N3CCC4(CC3)CCN(c3ccc(C5CCC(=O)NC5=O)cc3)CC4)cc2)C1 ; CN1C[C@H](Nc2cnn(C)c(=O)c2Br)C[C@H](c2ccc(C(=O)N3CCC4(CC3)CCN(c3ccc([C@@H]5CCC(=O)NC5=O)cc3)CC4)cc2)C1 ; CN1C[C@H](Nc2cnn(C)c(=O)c2Br)C[C@H](c2ccc(C(=O)N3CCC4(CC3)CCN(c3ccc([C@H]5CCC(=O)NC5=O)cc3)CC4)cc2)C1] 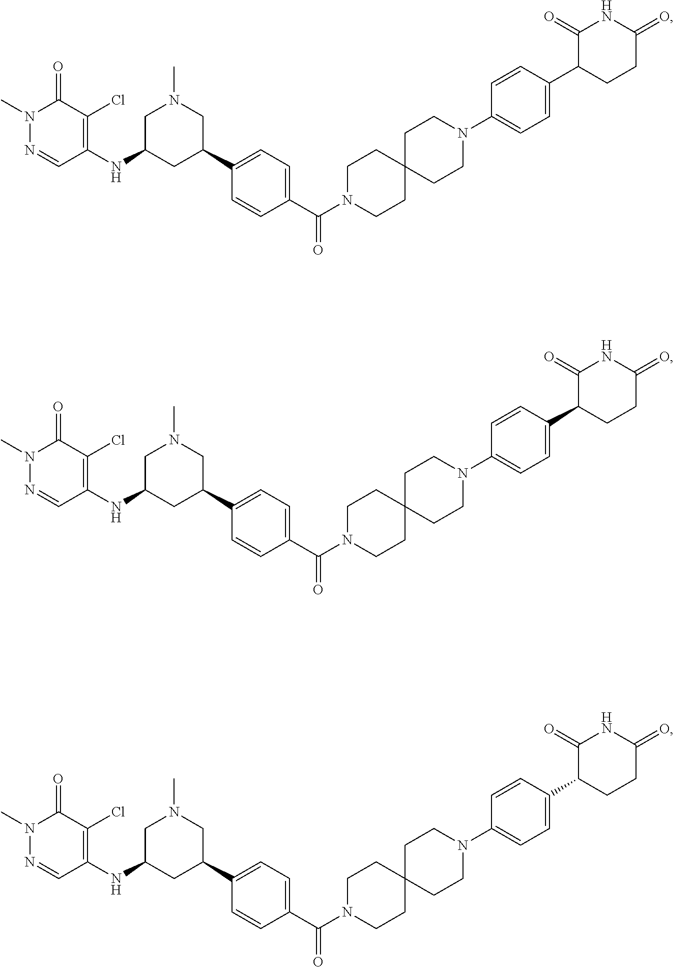 [SMILES: CN1C[C@H](Nc2cnn(C)c(=O)c2Cl)C[C@H](c2ccc(C(=O)N3CCC4(CC3)CCN(c3ccc(C5CCC(=O)NC5=O)cc3)CC4)cc2)C1 ; CN1C[C@H](Nc2cnn(C)c(=O)c2Cl)C[C@H](c2ccc(C(=O)N3CCC4(CC3)CCN(c3ccc([C@@H]5CCC(=O)NC5=O)cc3)CC4)cc2)C1 ; CN1C[C@H](Nc2cnn(C)c(=O)c2Cl)C[C@H](c2ccc(C(=O)N3CCC4(CC3)CCN(c3ccc([C@H]5CCC(=O)NC5=O)cc3)CC4)cc2)C1] 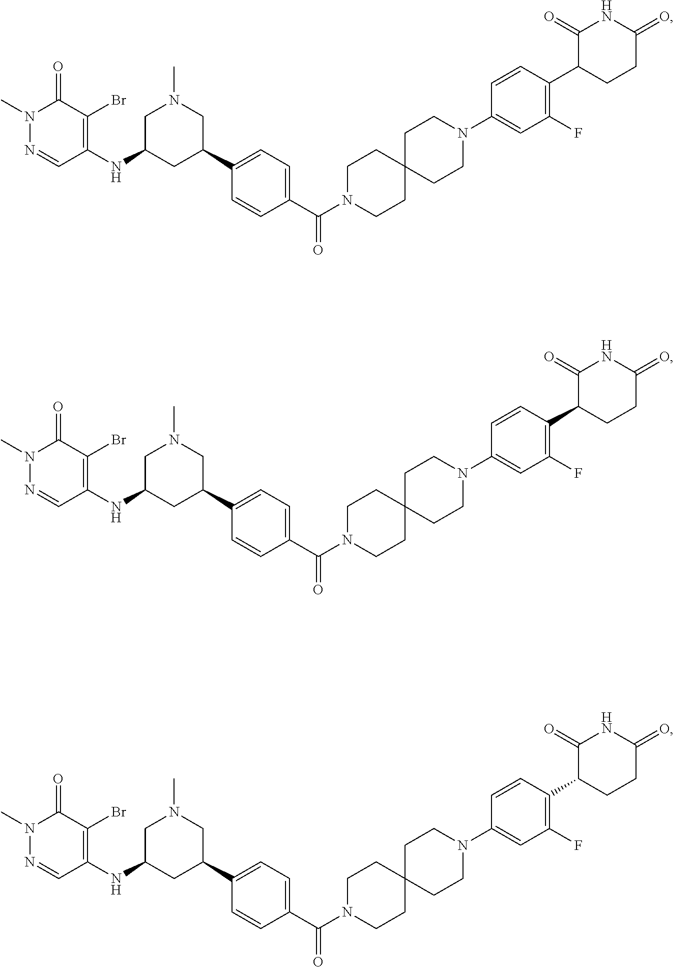 [SMILES: CN1C[C@H](Nc2cnn(C)c(=O)c2Br)C[C@H](c2ccc(C(=O)N3CCC4(CC3)CCN(c3ccc(C5CCC(=O)NC5=O)c(F)c3)CC4)cc2)C1 ; CN1C[C@H](Nc2cnn(C)c(=O)c2Br)C[C@H](c2ccc(C(=O)N3CCC4(CC3)CCN(c3ccc([C@@H]5CCC(=O)NC5=O)c(F)c3)CC4)cc2)C1 ; CN1C[C@H](Nc2cnn(C)c(=O)c2Br)C[C@H](c2ccc(C(=O)N3CCC4(CC3)CCN(c3ccc([C@H]5CCC(=O)NC5=O)c(F)c3)CC4)cc2)C1] 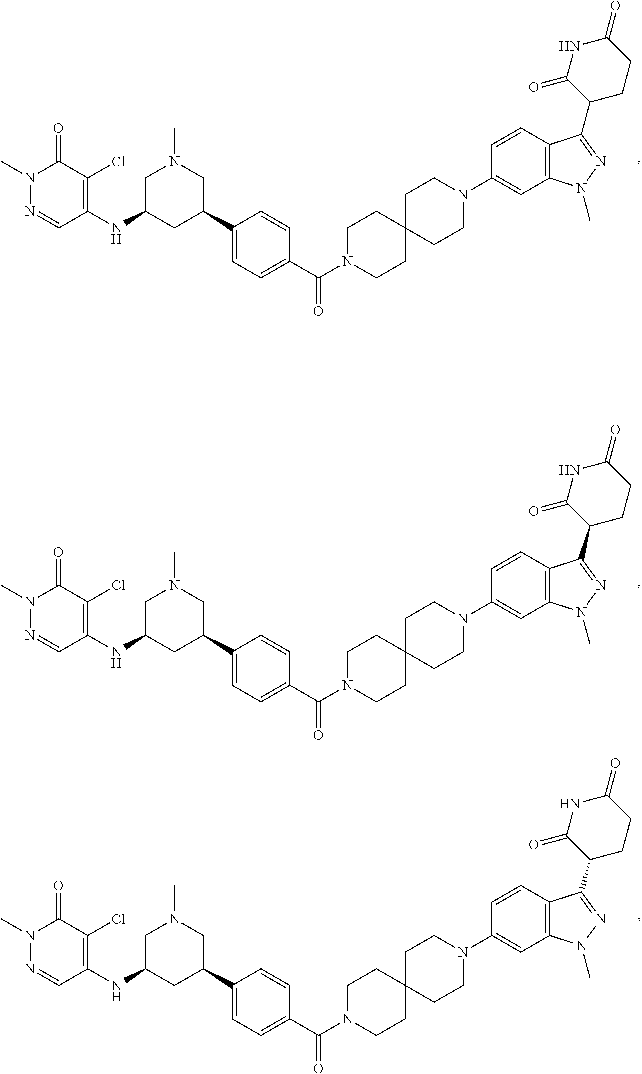 [SMILES: CN1C[C@H](Nc2cnn(C)c(=O)c2Cl)C[C@H](c2ccc(C(=O)N3CCC4(CC3)CCN(c3ccc5c(C6CCC(=O)NC6=O)nn(C)c5c3)CC4)cc2)C1 ; CN1C[C@H](Nc2cnn(C)c(=O)c2Cl)C[C@H](c2ccc(C(=O)N3CCC4(CC3)CCN(c3ccc5c([C@@H]6CCC(=O)NC6=O)nn(C)c5c3)CC4)cc2)C1 ; CN1C[C@H](Nc2cnn(C)c(=O)c2Cl)C[C@H](c2ccc(C(=O)N3CCC4(CC3)CCN(c3ccc5c([C@H]6CCC(=O)NC6=O)nn(C)c5c3)CC4)cc2)C1] 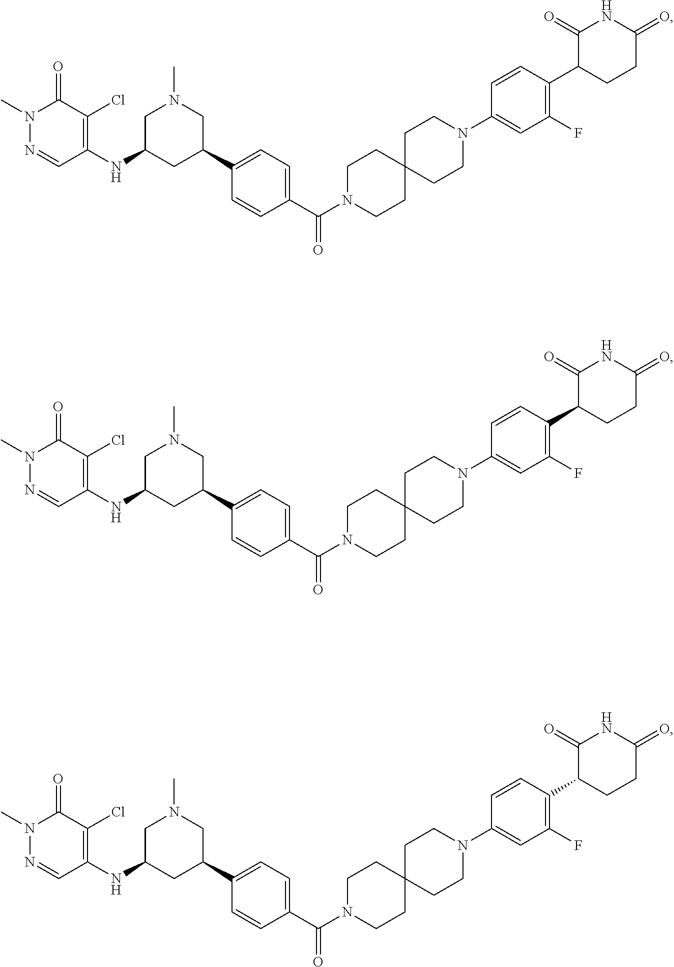 [SMILES: CN1C[C@H](Nc2cnn(C)c(=O)c2Cl)C[C@H](c2ccc(C(=O)N3CCC4(CC3)CCN(c3ccc(C5CCC(=O)NC5=O)c(F)c3)CC4)cc2)C1 ; CN1C[C@H](Nc2cnn(C)c(=O)c2Cl)C[C@H](c2ccc(C(=O)N3CCC4(CC3)CCN(c3ccc([C@@H]5CCC(=O)NC5=O)c(F)c3)CC4)cc2)C1 ; CN1C[C@H](Nc2cnn(C)c(=O)c2Cl)C[C@H](c2ccc(C(=O)N3CCC4(CC3)CCN(c3ccc([C@H]5CCC(=O)NC5=O)c(F)c3)CC4)cc2)C1] 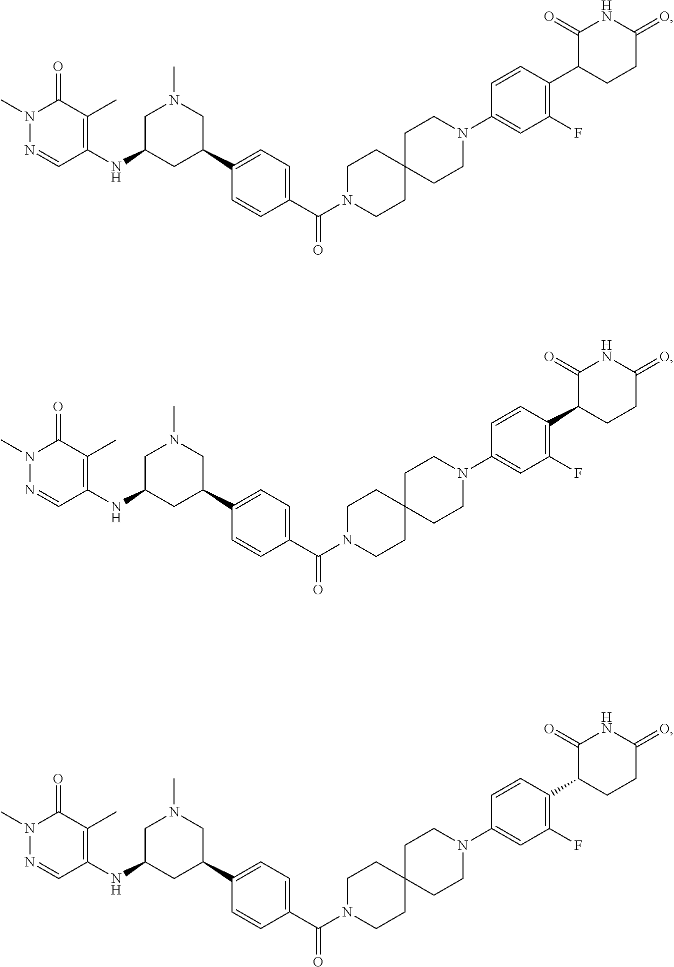 [SMILES: Cc1c(N[C@@H]2C[C@H](c3ccc(C(=O)N4CCC5(CC4)CCN(c4ccc(C6CCC(=O)NC6=O)c(F)c4)CC5)cc3)CN(C)C2)cnn(C)c1=O ; Cc1c(N[C@@H]2C[C@H](c3ccc(C(=O)N4CCC5(CC4)CCN(c4ccc([C@@H]6CCC(=O)NC6=O)c(F)c4)CC5)cc3)CN(C)C2)cnn(C)c1=O ; Cc1c(N[C@@H]2C[C@H](c3ccc(C(=O)N4CCC5(CC4)CCN(c4ccc([C@H]6CCC(=O)NC6=O)c(F)c4)CC5)cc3)CN(C)C2)cnn(C)c1=O] 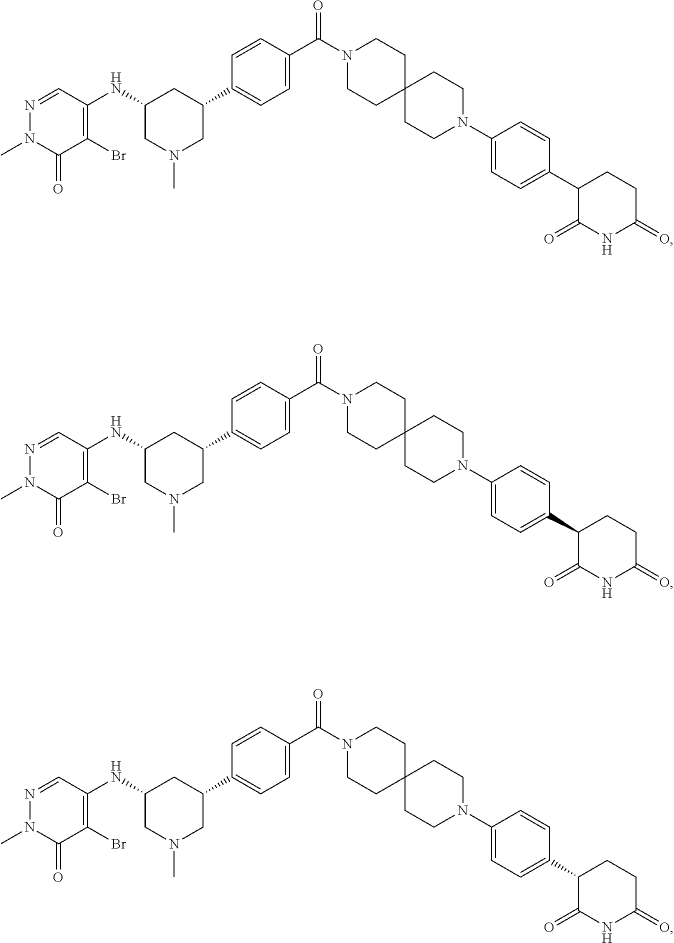 [SMILES: CN1C[C@H](Nc2cnn(C)c(=O)c2Br)C[C@H](c2ccc(C(=O)N3CCC4(CC3)CCN(c3ccc(C5CCC(=O)NC5=O)cc3)CC4)cc2)C1 ; CN1C[C@H](Nc2cnn(C)c(=O)c2Br)C[C@H](c2ccc(C(=O)N3CCC4(CC3)CCN(c3ccc([C@H]5CCC(=O)NC5=O)cc3)CC4)cc2)C1 ; CN1C[C@H](Nc2cnn(C)c(=O)c2Br)C[C@H](c2ccc(C(=O)N3CCC4(CC3)CCN(c3ccc([C@@H]5CCC(=O)NC5=O)cc3)CC4)cc2)C1] 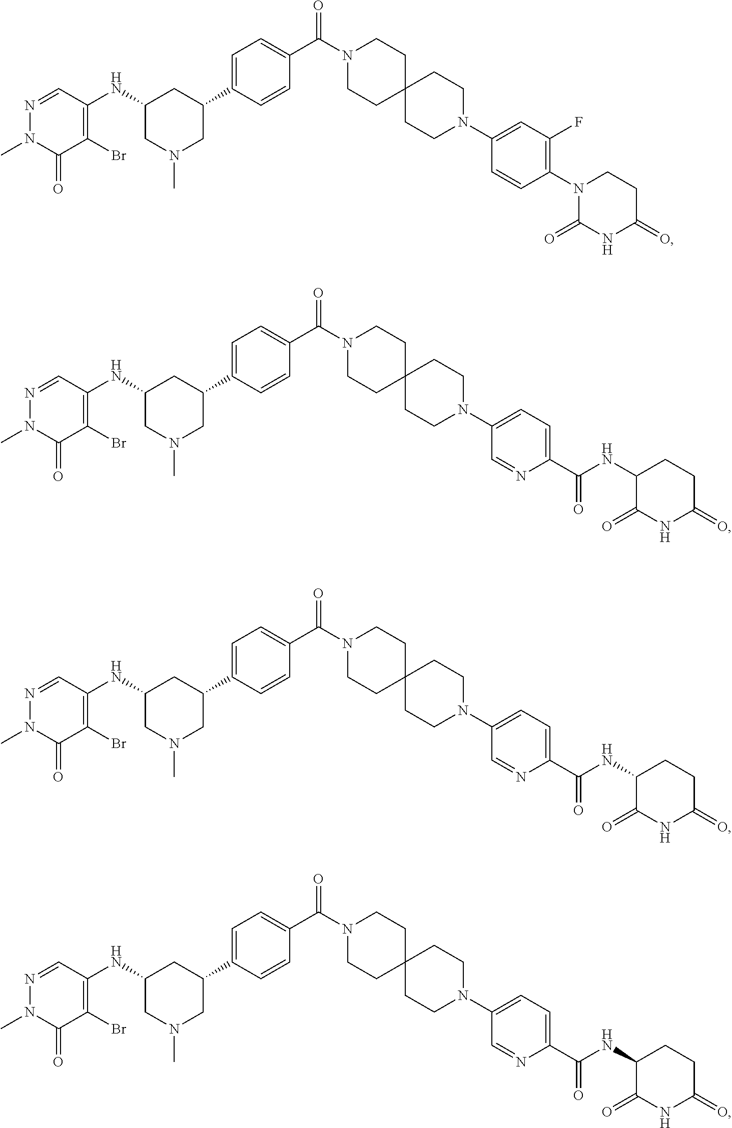 [SMILES: CN1C[C@H](Nc2cnn(C)c(=O)c2Br)C[C@H](c2ccc(C(=O)N3CCC4(CC3)CCN(c3ccc(N5CCC(=O)NC5=O)c(F)c3)CC4)cc2)C1 ; CN1C[C@H](Nc2cnn(C)c(=O)c2Br)C[C@H](c2ccc(C(=O)N3CCC4(CC3)CCN(c3ccc(C(=O)NC5CCC(=O)NC5=O)nc3)CC4)cc2)C1 ; CN1C[C@H](Nc2cnn(C)c(=O)c2Br)C[C@H](c2ccc(C(=O)N3CCC4(CC3)CCN(c3ccc(C(=O)N[C@@H]5CCC(=O)NC5=O)nc3)CC4)cc2)C1 ; CN1C[C@H](Nc2cnn(C)c(=O)c2Br)C[C@H](c2ccc(C(=O)N3CCC4(CC3)CCN(c3ccc(C(=O)N[C@H]5CCC(=O)NC5=O)nc3)CC4)cc2)C1] 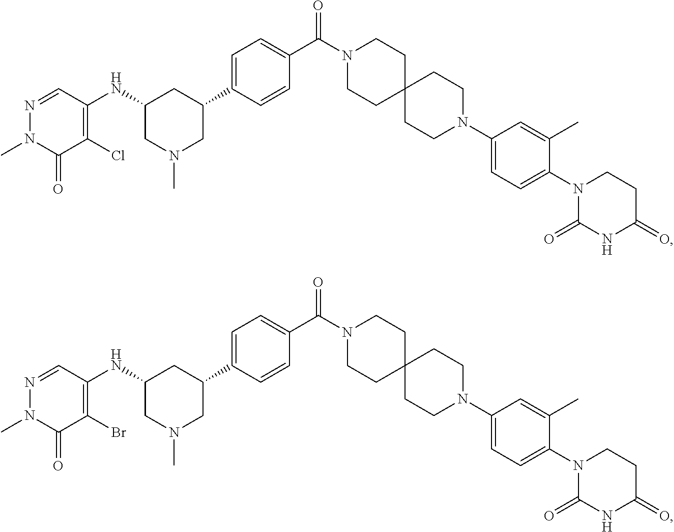 [SMILES: Cc1cc(N2CCC3(CCN(C(=O)c4ccc([C@H]5C[C@@H](Nc6cnn(C)c(=O)c6Cl)CN(C)C5)cc4)CC3)CC2)ccc1N1CCC(=O)NC1=O ; Cc1cc(N2CCC3(CCN(C(=O)c4ccc([C@H]5C[C@@H](Nc6cnn(C)c(=O)c6Br)CN(C)C5)cc4)CC3)CC2)ccc1N1CCC(=O)NC1=O] 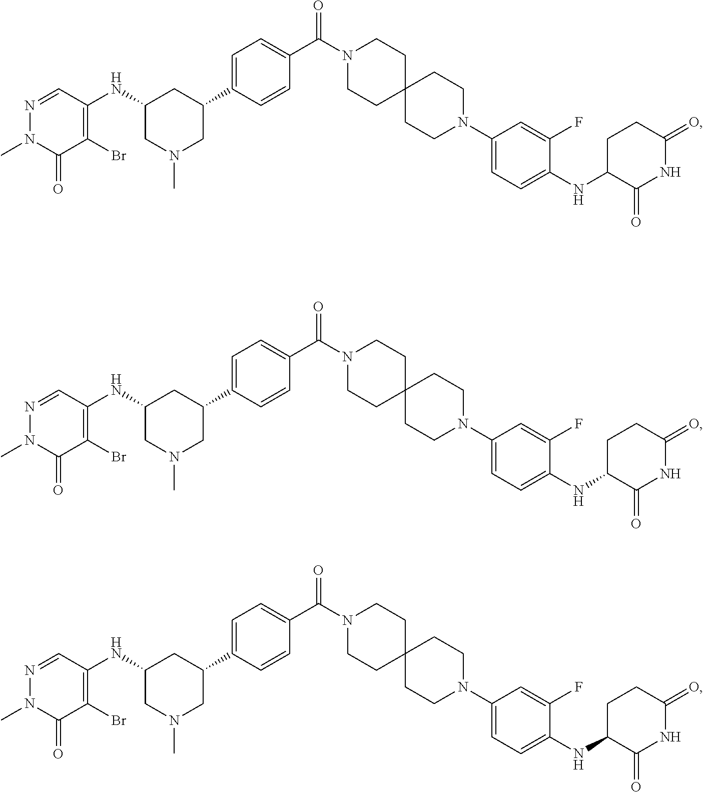 [SMILES: CN1C[C@H](Nc2cnn(C)c(=O)c2Br)C[C@H](c2ccc(C(=O)N3CCC4(CC3)CCN(c3ccc(NC5CCC(=O)NC5=O)c(F)c3)CC4)cc2)C1 ; CN1C[C@H](Nc2cnn(C)c(=O)c2Br)C[C@H](c2ccc(C(=O)N3CCC4(CC3)CCN(c3ccc(N[C@@H]5CCC(=O)NC5=O)c(F)c3)CC4)cc2)C1 ; CN1C[C@H](Nc2cnn(C)c(=O)c2Br)C[C@H](c2ccc(C(=O)N3CCC4(CC3)CCN(c3ccc(N[C@H]5CCC(=O)NC5=O)c(F)c3)CC4)cc2)C1] 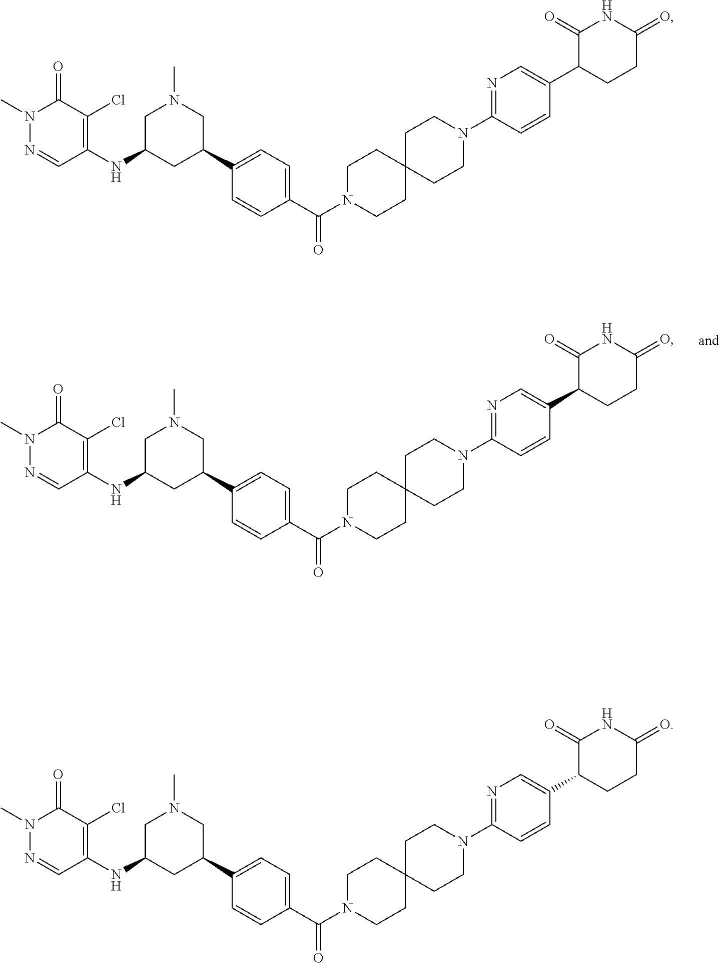 [SMILES: CN1C[C@H](Nc2cnn(C)c(=O)c2Cl)C[C@H](c2ccc(C(=O)N3CCC4(CC3)CCN(c3ccc(C5CCC(=O)NC5=O)cn3)CC4)cc2)C1 ; CN1C[C@H](Nc2cnn(C)c(=O)c2Cl)C[C@H](c2ccc(C(=O)N3CCC4(CC3)CCN(c3ccc([C@@H]5CCC(=O)NC5=O)cn3)CC4)cc2)C1 ; CN1C[C@H](Nc2cnn(C)c(=O)c2Cl)C[C@H](c2ccc(C(=O)N3CCC4(CC3)CCN(c3ccc([C@H]5CCC(=O)NC5=O)cn3)CC4)cc2)C1]
or a pharmaceutically acceptable salt thereof.

2. A compound of Formula I: 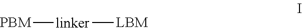
or a pharmaceutically acceptable salt thereof, wherein;
PBM is a KAT2 protein binding moiety, or is 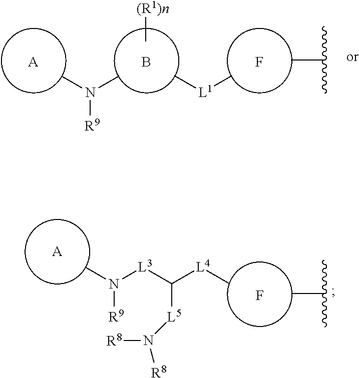
linker is an optional linking moiety, or is a covalent bond or an optionally substituted bivalent C₁-C₂₀ hydrocarbon chain, wherein one or more methylene units are optionally and independently replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —S(O)—, —SO₂—, —SO₂N(R)—, —N(R)SO₂—, or -Cy-; and
LBM is an E3 ubiquitin ligase binding moiety, or is 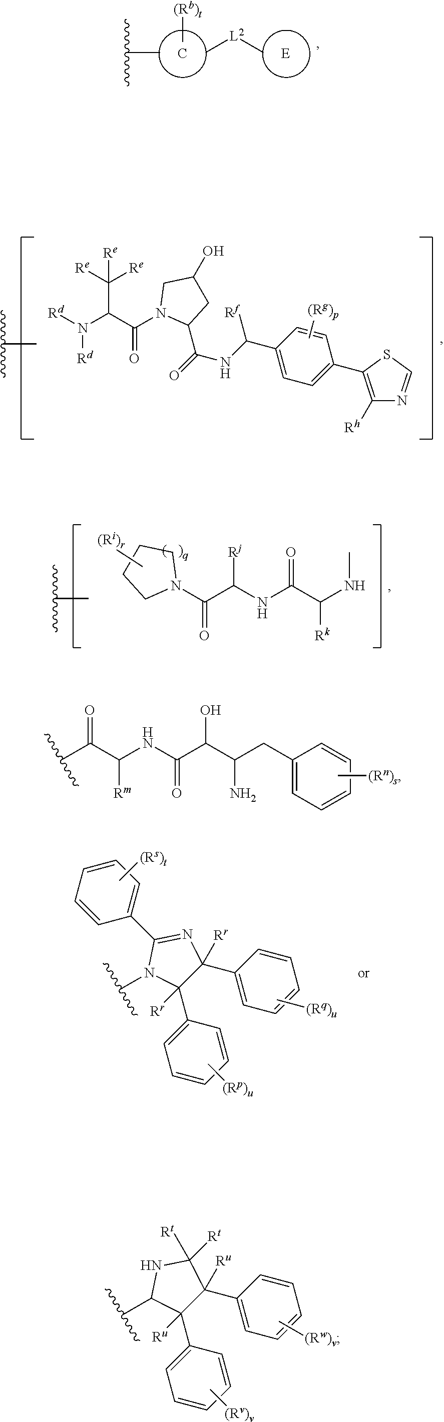
wherein:
Ring A is selected from: 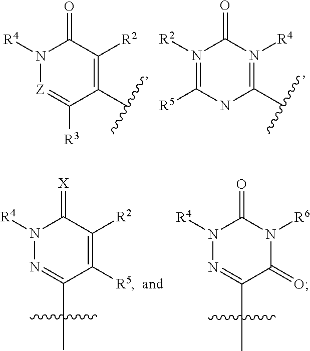
Ring B is an optionally substituted 5- to 6-membered heterocyclyl having 1-2 heteroatoms independently selected from N, O, and S;
L¹ is a covalent bond or an optionally substituted bivalent C₁₋₃ hydrocarbon chain;
each R¹ is independently —R;
n is 0, 1, 2, 3, or 4;
Z is N or CR³;
R² is halogen, —CN, or —R;
each R³ is independently halogen, —CN, or —R;
R⁴ is —R′;
R⁵ is halogen, —CN, or —R, or
R² and R⁵, together with the atoms to which they are attached, combine to form an optionally substituted 5- to 6-membered ring having 0-2 heteroatoms independently selected from N, O, and S;
R⁶ is —R′;
X is O or NR⁷;
R⁷ is —R′, or
R⁴ and R⁷, together with the atoms to which they are attached, combine to form an optionally substituted 5- to 6-membered ring having 2-3 heteroatoms independently selected from N, O, and S;
L³, L⁴, and L⁵ are each independently a covalent bond or an optionally substituted bivalent C₁₋₆ hydrocarbon chain;
each R⁸ is independently —R′;
R⁹ is —R′;
each Cy is independently an optionally substituted, 3- to 16-membered bivalent ring having 0-6 heteroatoms independently selected from N, O, and S;
each of Ring C and Ring F is independently an optionally substituted 3- to 16-membered ring having 0-6 heteroatoms independently selected from N, O, and S;
each R^{b} is independently halogen, —CN, —R′ or —OR;
t is 0, 1, 2, 3, or 4;
L² is a covalent bond or an optionally substituted C₁₋₃ hydrocarbon chain, wherein one or more methylene units are optionally and independently replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —S(O)—, —SO₂—, —SO₂N(R)—, —N(R)SO₂—, or -Cy-;
Ring E is 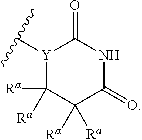
or an optionally substituted group selected from 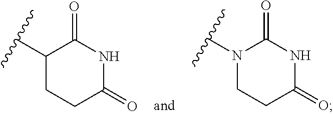 [SMILES: *C1CCC(=O)NC1=O ; *N1CCC(=O)NC1=O]
Y is N or CH;
each R^{a} is independently halogen, —CN or —R′;
linker is attached to the bracketed moiety at one of R^{d}, R^{e}, R^{f}, or R^{g}, or linker is attached to the bracketed moiety at one Rⁱ or the ring formed when two Rⁱ groups are taken together;
each R^{d} that is not the point of attachment for the linker is independently —R′, or
two R^{d}, together with the atom to which they are attached, combine to form an optionally substituted 5- to 6-membered ring having 1-3 heteroatoms independently selected from N, O, and S and optionally fused to a phenyl or 5- to 6-membered heteroaryl ring having 1-3 heteroatoms independently selected from N, O, and S;
each R^{e} that is not the point of attachment for the linker is independently —R;
R^{f}, when it is not the point of attachment for the linker, is —R;
each R^{g} that is not the point of attachment for the linker is independently halogen, —CN, R or —OR;
p is 0, 1, 2, or 3;
R^{h} is halogen, or —R;
each Rⁱ that is not the point of attachment of the linker is independently halogen, —R, —C(O)N(R′)₂, or —N(R)C(O)R, or
two Rⁱ groups, together with the atoms to which they are attached, combine to form an optionally substituted phenyl or 5- to 6-membered ring having 1-3 heteroatoms independently selected from N, O, and S;
R^{j} is —R, or
R^{j} combines with one instance of Rⁱ, together with their intervening atoms, to form an optionally substituted 5- to 7-membered ring having 1-2 heteroatoms independently selected from N, O, and S;
R^{k} is —R;
r is 1, 2, 3, 4, or 5;
q is 1 or 2;
R^{m} is —R;
each of Rⁿ, R^{p}, R^{q}, R^{r}, R^{s}, and R^{u} is independently halogen, —CN, —R or —OR;
s is 0, 1, 2, 3, 4, or 5;
t is 0, 1, 2, 3, 4, or 5;
each u is independently 0, 1, 2, 3, 4, or 5;
each R^{t} is independently R, or
both R^{t} groups, together with the atom to which they are attached, combine to form an optionally substituted 3- to 7-membered ring having 0-2 heteroatoms independently selected from N, O, and S;
each R^{v} is independently halogen, —CN, —R or —OR, or
one instance of R^{u} and R^{v}, together with their intervening atoms, combine to form an optionally substituted 3- to 7-membered ring having 0-2 heteroatoms independently selected from N, O, and S;
each R^{w} is independently halogen, —CN, —R or —OR, or
one instance of R^{u} and R^{w}, together with their intervening atoms, combine to form an optionally substituted 3- to 7-membered ring having 0-2 heteroatoms independently selected from N, O, and S;
each v is independently 0, 1, 2, 3, 4, or 5;
each R′ is independently —R, —C(O)R, or —S(O)₂R, or two R′ attached to the same atom, together with the atom to which they are attached, combine to form an optionally substituted 3- to 16-membered ring having 1-5 heteroatoms independently selected from N, O, and S; and
each R is independently hydrogen or an optionally substituted group selected from C₁₋₈ aliphatic, C₃₋₁₀ cycloaliphatic, C₁-C₈ heteroaliphatic having 1-3 heteroatoms independently selected from N, O, and S, C₆₋₁₀ aryl, 5- to 10-membered heteroaryl having 1-5 heteroatoms independently selected from N, O, and S, and 3- to 10-membered monocyclic heterocyclyl having 1-5 heteroatoms independently selected from N, O, and S.

3. The compound of any one of the preceding claims, wherein the PBM is a KAT2A protein binding moiety and/or a KAT2B protein binding moiety.

4. The compound of any one of claims 2-3, wherein PBM is

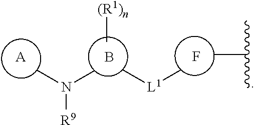

5. The compound of any one of claims 2-4, wherein Ring F is an optionally substituted phenyl ring.

6. The compound of any one of claims 2-5, wherein R² is halogen.

7. The compound of any one of claims 2-5, wherein Ring A is

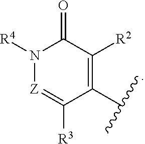

8. The compound of claim 7, wherein Ring A is selected from

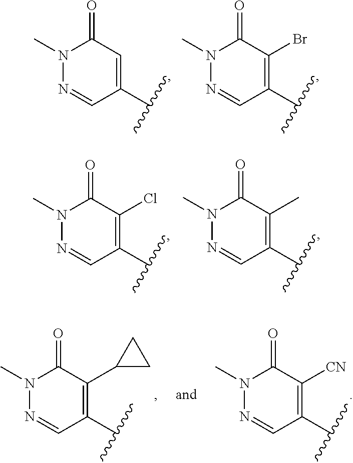

9. The compound of any one of claims 2-5, wherein Ring A is

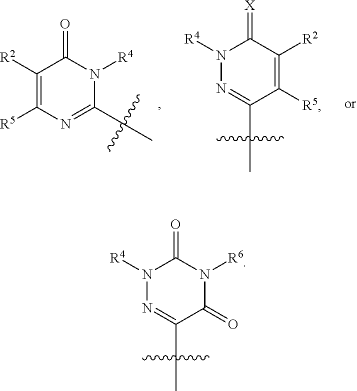

10. The compound of claim 9, wherein R⁶ is optionally substituted C₁₋₆ aliphatic and/or wherein R⁴ is optionally substituted C₁₋₆ alkyl.

11. The compound of any one of claims 2-5, wherein Ring A is selected from

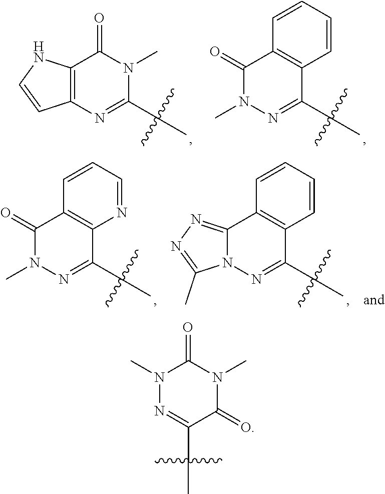

12. The compound of any one of claims 2-11, wherein Ring B is an optionally substituted 6-membered heterocyclyl having 1-2 heteroatoms independently selected from N, O, and S.

13. The compound of any one of claims 2-11, wherein Ring B is optionally substituted

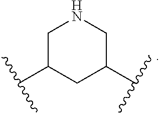 [SMILES: *C1CNCC(*)C1]

14. The compound of any one of claims 2-11, wherein 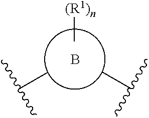
is

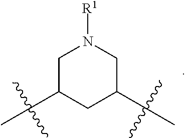

15. The compound of any one of claims 2-14, wherein each R¹ is independently optionally substituted C₁₋₆ alkyl, preferably methyl.

16. The compound of any one of claims 2-15, wherein L¹ is a covalent bond.

17. The compound of any one of claims 2-16, wherein 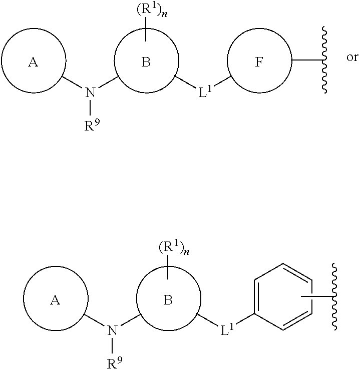
is selected from

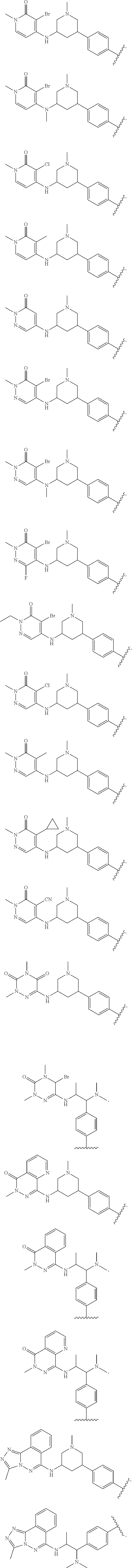

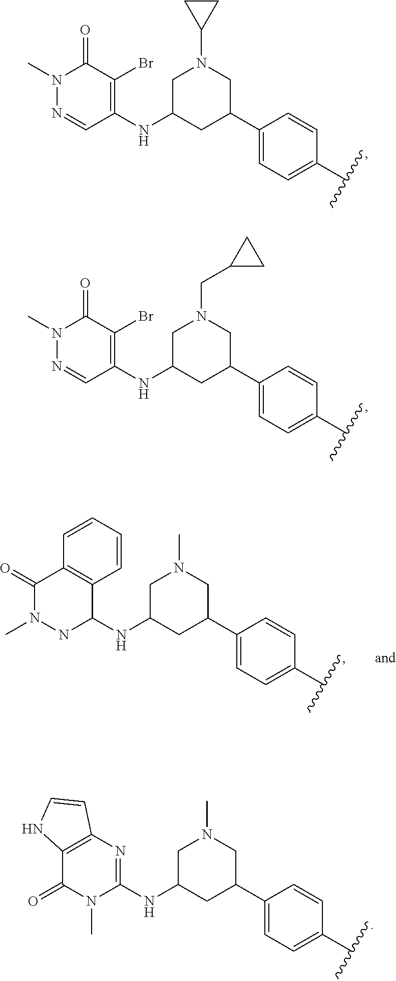 [SMILES: *c1ccc(C2CC(Nc3cnn(C)c(=O)c3Br)CN(C3CC3)C2)cc1 ; *c1ccc(C2CC(Nc3cnn(C)c(=O)c3Br)CN(CC3CC3)C2)cc1 ; *c1ccc(C2CC(NC3NN(C)C(=O)c4ccccc43)CN(C)C2)cc1 ; *c1ccc(C2CC(Nc3nc4cc[nH]c4c(=O)n3C)CN(C)C2)cc1]

18. The compound of any one of the preceding claims, wherein the linker is less than 14 atoms in length, or wherein the linker is less than 11 atoms in length; and/or
wherein the shortest-path length of linker is about or no more than 14 atoms, or wherein the shortest-path length of linker is about or no more than 10 atoms; and/or wherein the number of non-ring consecutive sp³ atoms in the shortest path chain of linker is about or no more than about 5, 4, 3, 2 or 1; and/or wherein the number of non-ring consecutive sp³ atoms in the shortest path chain of linker is about or no more than about 2; and/or wherein the number of bonds between two non-ring sp³ atoms in the shortest path chain of linker is about or no more than 5, 4, 3, 2 or 1; and/or wherein the number of bonds between two non-ring sp³ atoms in the shortest path chain of linker is about or no more than 1; and/or wherein the number of non-ring sp³ C, O, and S atoms in the shortest path chain of linker is about or no more than about 5, 4, 3, 2 or 1; and/or wherein the number of non-ring sp³ atoms in the shortest path chain of linker is about or no more than about 5, 4, 3, 2 or 1; and/or wherein the number of non-ring sp³ atoms in the shortest path chain of linker is about or no more than about 1; and/or wherein the number of non-ring atoms in the shortest path chain of linker is about or no more than about 5, 4, 3, 2 or 1; and/or wherein the number of non-ring atoms in the shortest path chain of linker is about or no more than about 1.

19. The compound of any one of claims 2-17, wherein linker is an optionally substituted, bivalent, straight or branched, saturated or unsaturated C₁-C₂₀ hydrocarbon chain, wherein one or more methylene units are optionally and independently replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —SO₂—, —SO₂N(R)—, —N(R)SO₂—, or -Cy-, preferably:
wherein linker is an optionally substituted, bivalent, straight or branched, saturated or unsaturated C₁-C₂₀ hydrocarbon chain, wherein at least one methylene unit is replaced by —O—, —N(R)—, —C(O)—, —OC(O)—, —C(O)O—, —C(O)N(R)—, —N(R)C(O)—, or -Cy-; and
wherein a methylene unit is replaced by —C(O)—, a methylene unit is replaced by -Cy-, preferably wherein -Cy- is in a shortest path chain, and/or a methylene unit is replaced by —C(O)N(R)—.

20. The compound of any one of claims 2-17, wherein linker is: 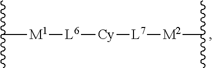
wherein:
M¹ and M² are each independently absent, —CH₂—, —O—, —N(R)—, —C(O)—, —OC(O)—, —C(O)O—, —C(O)N(R)—, or —N(R)C(O)—; and
L⁶ and L⁷ are each independently a covalent bond or an optionally substituted, bivalent, straight or branched, saturated or unsaturated C₁-C₁₀ hydrocarbon chain, wherein one or more methylene units are optionally and independently replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —SO₂—, —SO₂N(R)—, —N(R)SO₂—, or -Cy-.

21. The compound of any one of claims 2-17, wherein linker is selected from

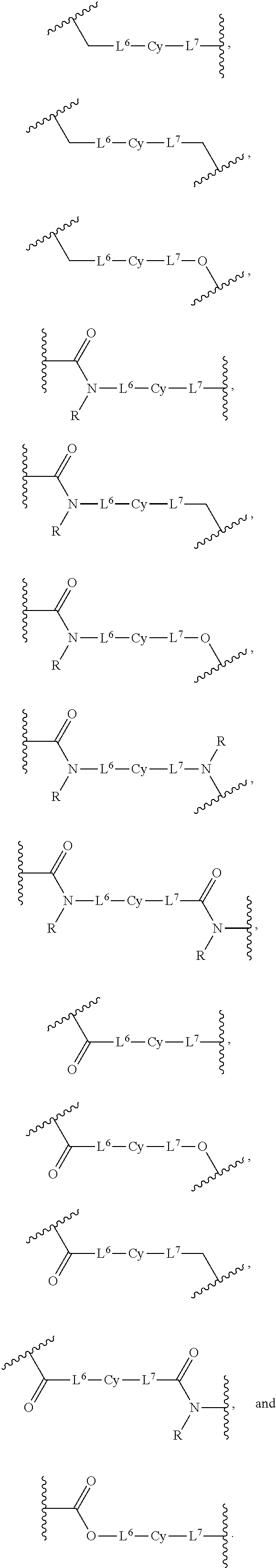

22. The compound of any one of claims 2-17, wherein -Cy- is monocyclic and optionally 3-10 membered, or wherein -Cy- is bicyclic optionally wherein -Cy- is 6-, 7-, 8-, 9-, 10- or 11-membered.

23. The compound of any one of claims 2-22, wherein each monocyclic ring unit independently has 1-2 heteroatoms independently selected from N, O, and S.

24. The compound of any one of claims 2-23, wherein Cy is optionally substituted monocyclic 5- to 6-membered heterocyclyl having 1-2 heteroatoms independently selected from N, O, and S.

25. The compound of any one of claims 2-23, wherein Cy is optionally substituted bicyclic or spirocyclic 6- to 11-membered heterocyclyl having 1-3 heteroatoms independently selected from N, O, and S.

26. The compound of any one of claims 2-20, wherein linker is selected from 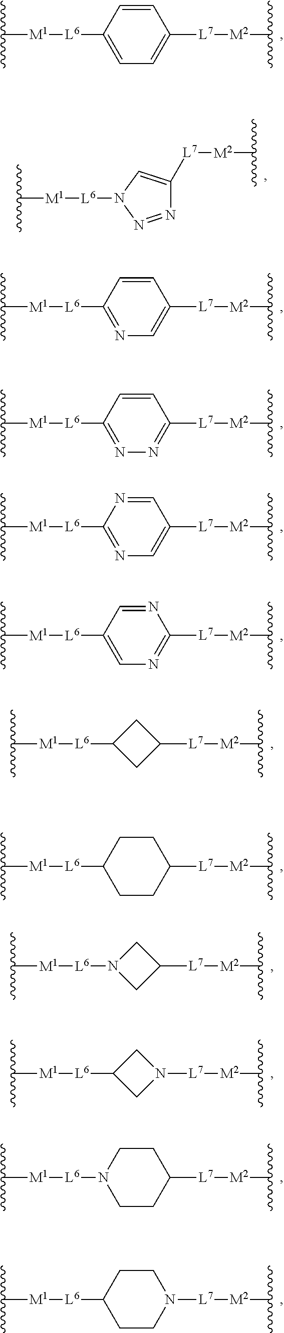 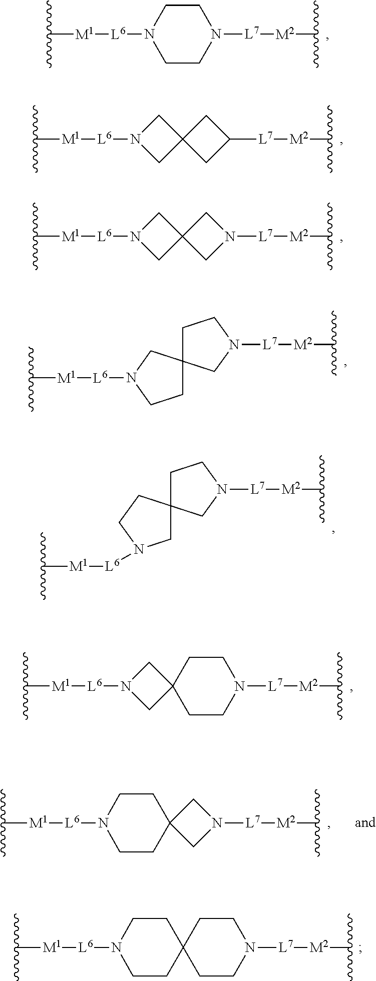
or wherein the linker is selected from: —CH₂—,

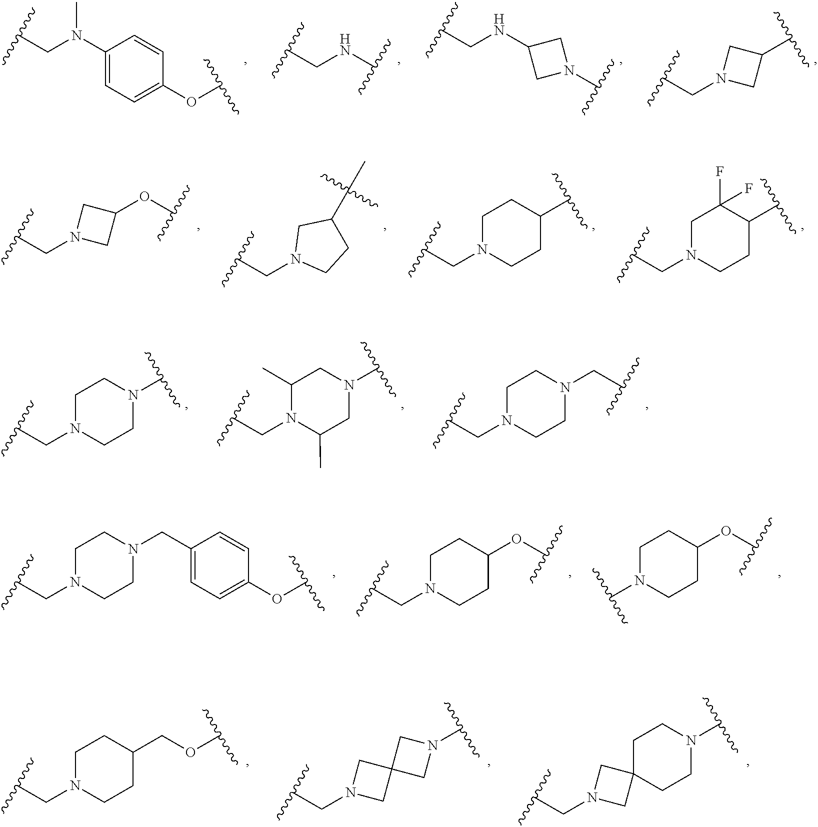

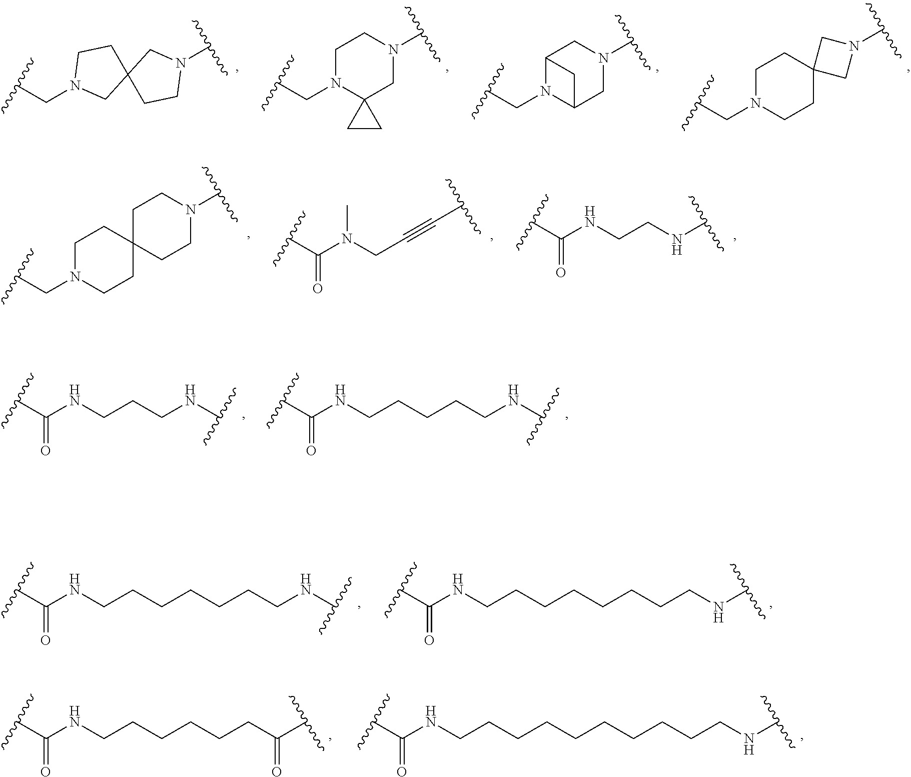

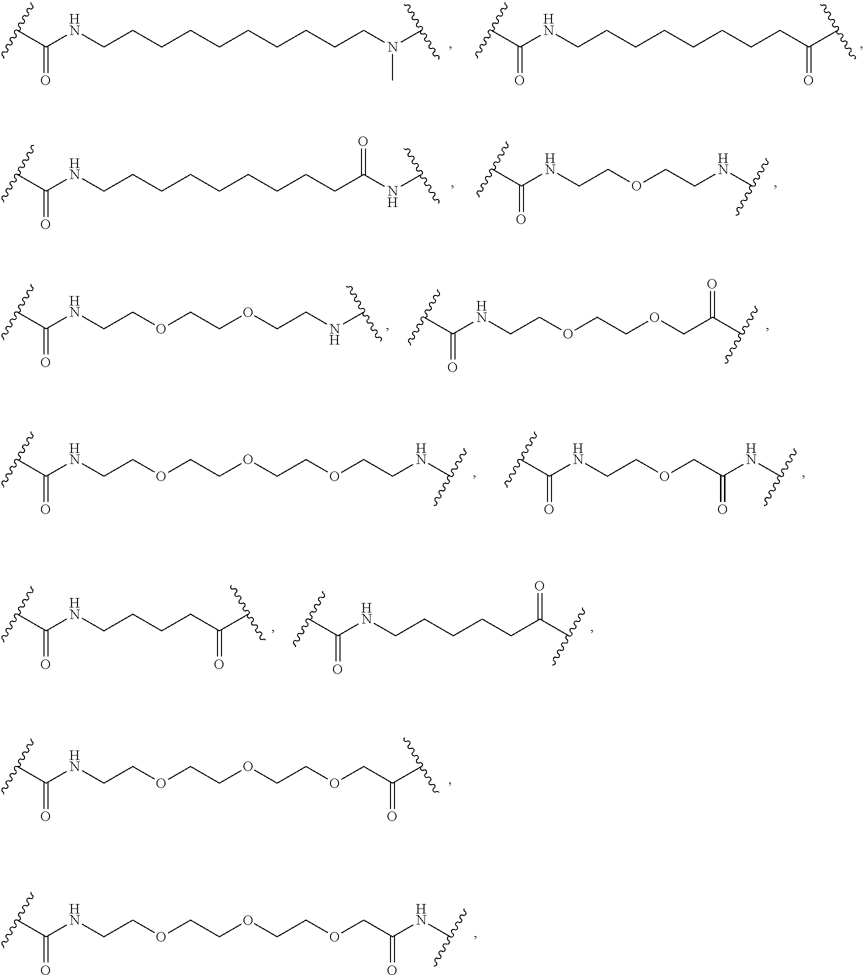

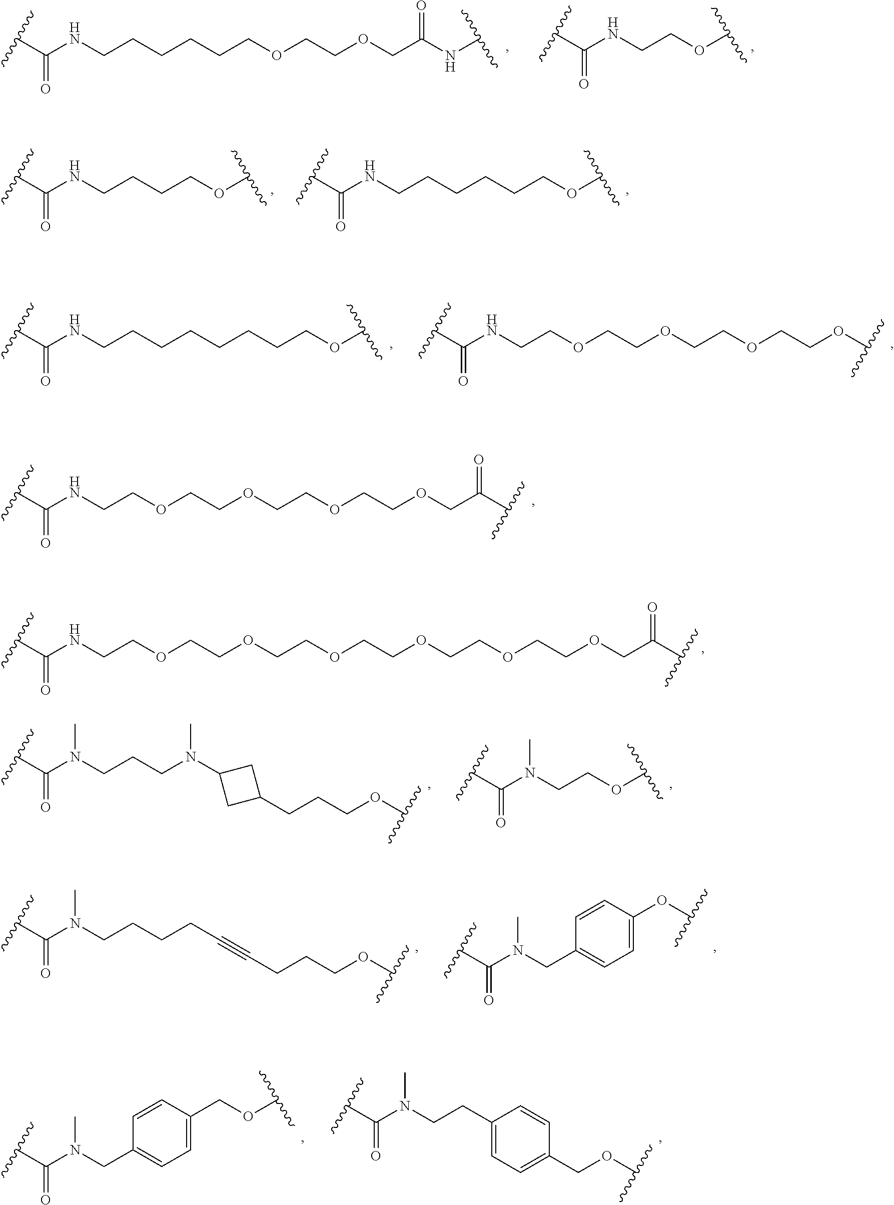

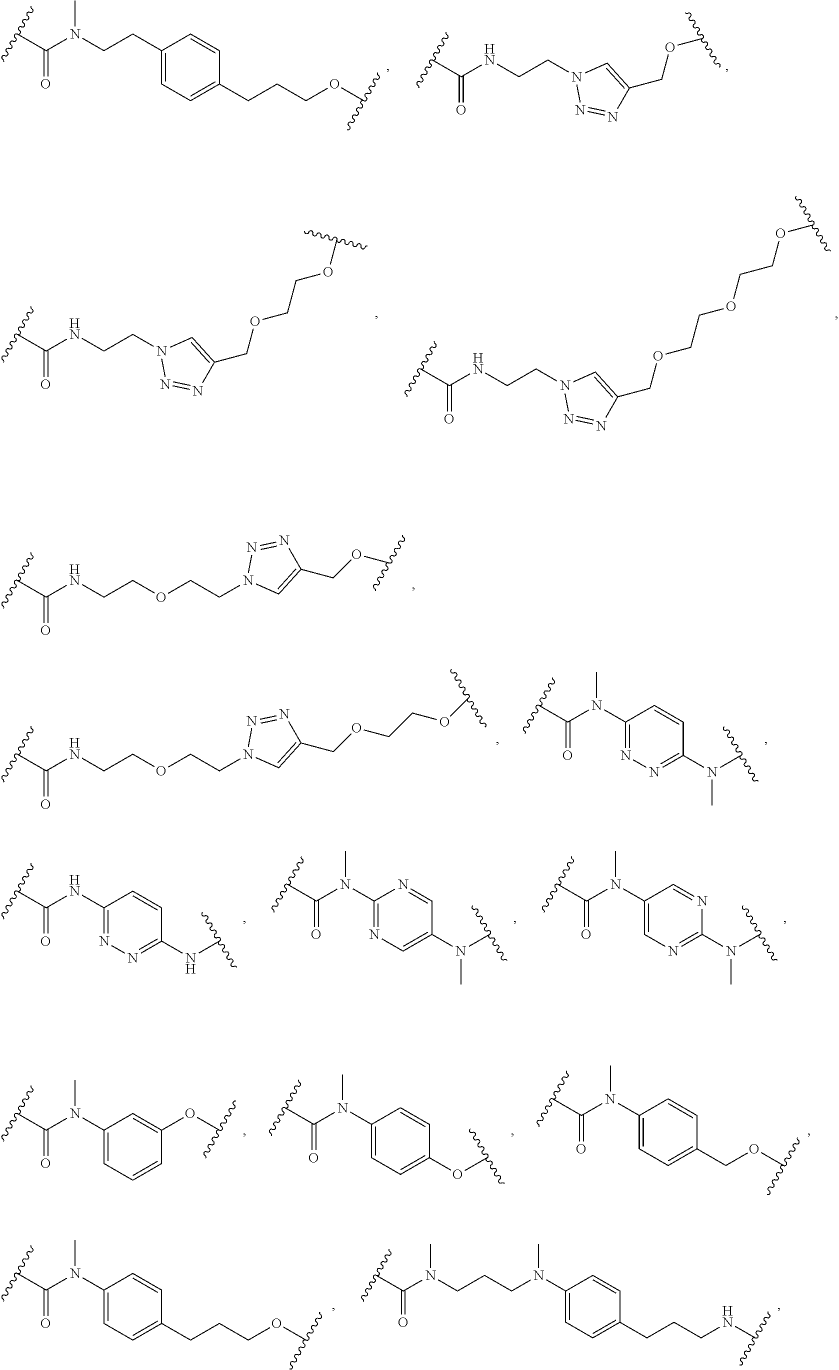

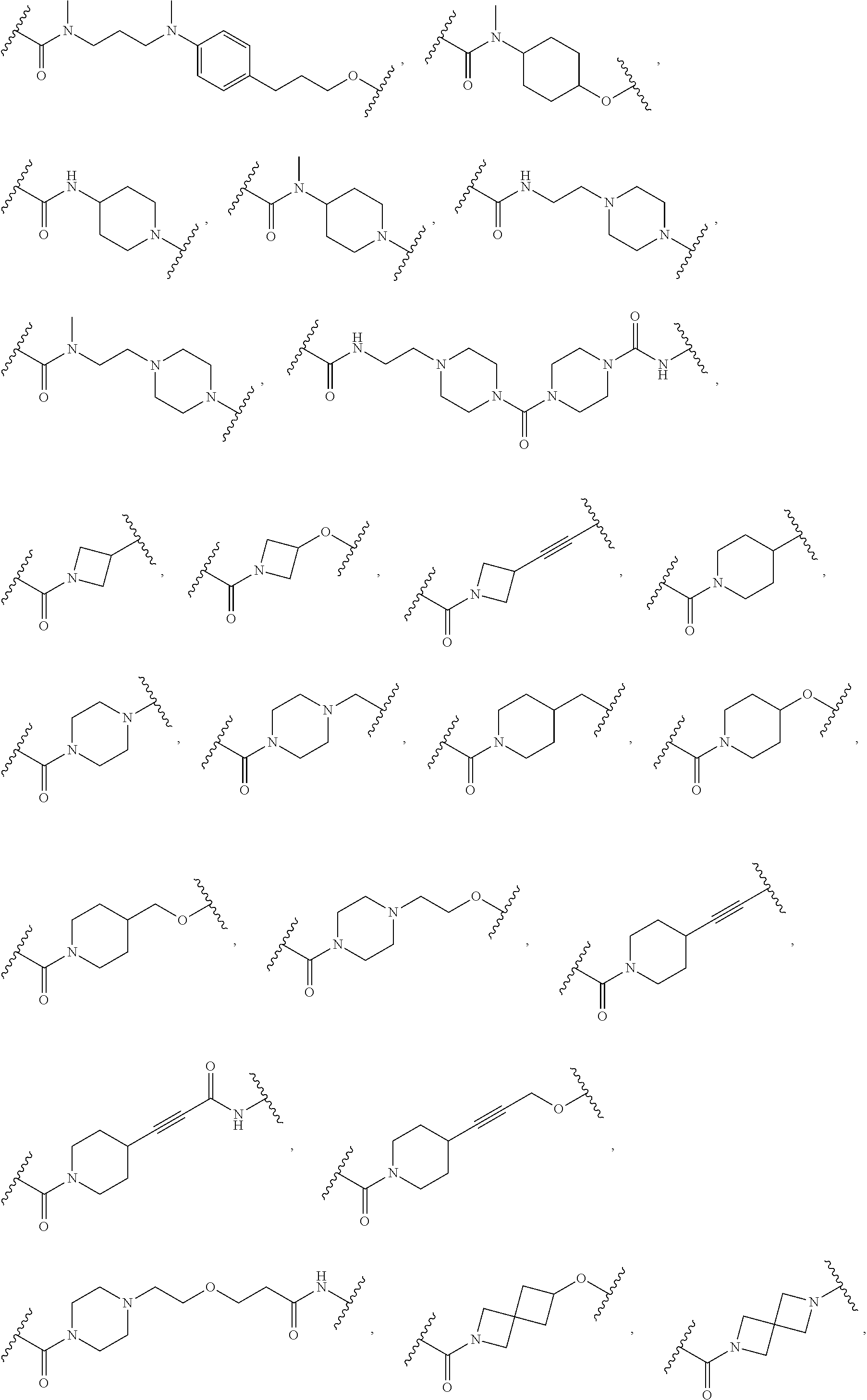

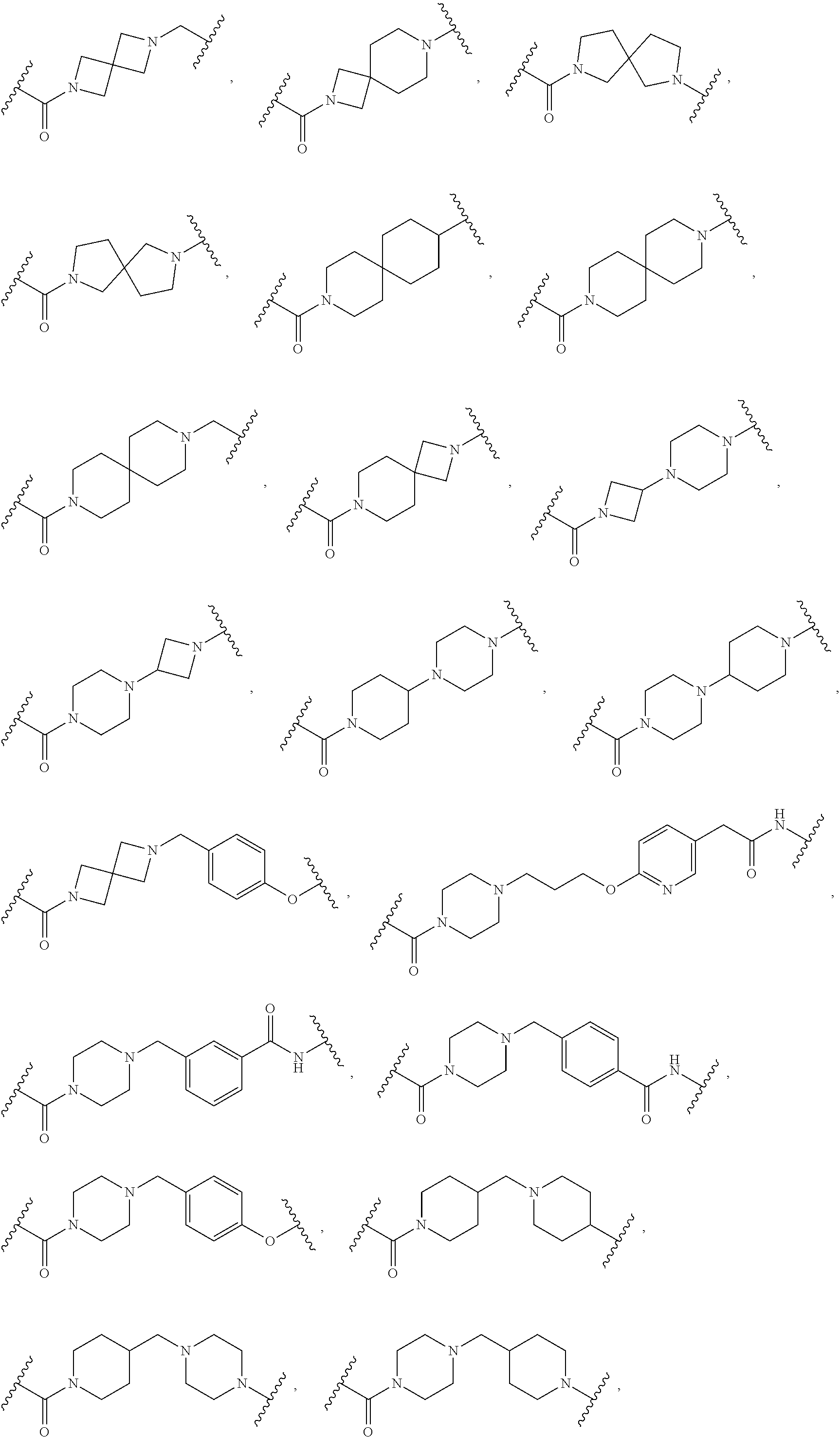

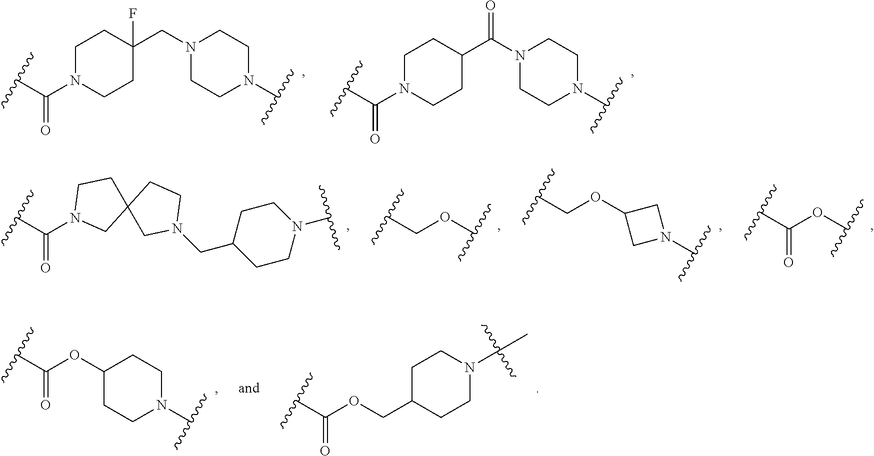

27. The compound of any one of claims 2-26, wherein LBM is an E3 ubiquitin ligase binding moiety, or wherein LBM is a VHL binding moiety, an IAP binding moiety, or a MDM2 binding moiety.

28. The compound of any one of claims 2-27, wherein LBM is

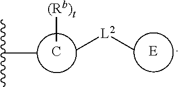

29. The compound of any one of claims 2-27, wherein the compound is of Formula III-1: 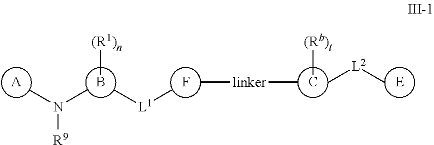
or a pharmaceutically acceptable salt thereof, wherein:
each R^{b} is independently halogen, —CN, —R′ or —OR;
t is 0, 1, 2, 3, or 4;
L² is a covalent bond or an optionally substituted C₁₋₃ hydrocarbon chain, wherein one or more methylene units are optionally and independently replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —S(O)—, —SO₂—, —SO₂N(R)—, —N(R)SO₂—, or -Cy-;
Ring E is 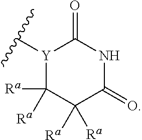
or an optionally substituted group selected from 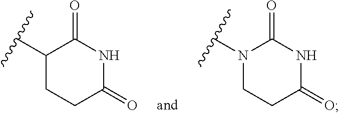 [SMILES: *C1CCC(=O)NC1=O ; *N1CCC(=O)NC1=O]
Y is N or CH; and
each R^{a} is independently halogen, —CN or —R′.

30. The compound of any one of claims 2-27, wherein LBM is

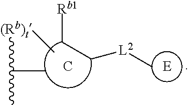

31. The compound of any one of claims 2-27, wherein the compound is of Formula IIIL: 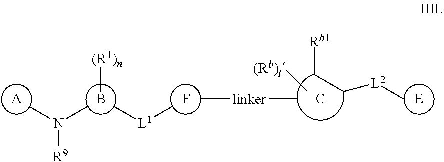
or a pharmaceutically acceptable salt thereof, wherein;
each R^{b} and R^{b1} is independently halogen, —CN, —R′ or —OR;
t′ is 0, 1, 2, or 3;
L² is a covalent bond or an optionally substituted C₁₋₃ hydrocarbon chain, wherein one or more methylene units are optionally and independently replaced by —O—, —S—, —N(R)—, —N═N—, —C(O)—, —C(S)—, —C(NR)—, —C(NOR)—, —C(NNR₂)—, —OC(O)—, —C(O)O—, —C(O)N(R)—, —N(R)C(O)—, —C(NR)O—, —OC(NR)—, —C(NR)NR—, —N(R)C(NR)—, —N(R)C(O)N(R)—, —N(R)C(O)O—, —OC(O)N(R)—, —N(R)C(O)S—, —SC(O)N(R)—, —N(R)C(NR)N(R)—, —S(O)—, —SO₂—, —SO₂N(R)—, —N(R)SO₂—, or -Cy-;
Ring E is 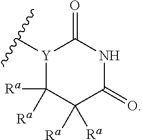
or an optionally substituted group selected from 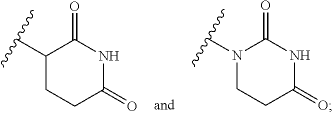 [SMILES: *C1CCC(=O)NC1=O ; *N1CCC(=O)NC1=O]
Y is N or CH; and
each R^{a} is independently halogen, —CN or —R′.

32. The compound of any one of claims 2-31, wherein R^{b1} is not —H, or wherein R^{b1} is optionally substituted C₁₋₆ aliphatic or halogen, or wherein R^{b1} is —OR wherein R is optionally substituted C₁₋₆ aliphatic.

33. The compound of any one of claims 2-31, wherein Ring C is an optionally substituted phenyl ring.

34. The compound of any one of claims 2-31, wherein Ring C is an optionally substituted 5- or 6-membered heteroaryl ring having 1-2 heteroatoms independently selected from N, O, and S, or Ring C is an optionally substituted 9- or 10-membered bicyclic heteroaryl ring having 1-4 heteroatoms independently selected from N, O, and S.

35. The compound of any one of claims 2-34, wherein Ring E is 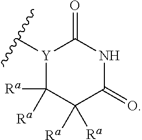
or wherein Ring E isoptionally substituted and selected from

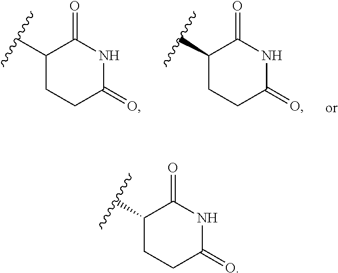 [SMILES: *C1CCC(=O)NC1=O ; *[C@@H]1CCC(=O)NC1=O ; *[C@H]1CCC(=O)NC1=O]

36. The compound of any one of claims 2-35, wherein Ring C is: 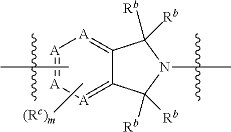
wherein:
each A is independently N, C, or CH, provided that no more than two A groups are N;
each R^{b} is hydrogen, or two R^{b} groups, on the same carbon, are taken together to form an oxo or combine to form a 3- to 6-membered saturated or partially unsaturated ring;
each R^{c} is independently selected from halogen, —OR, —N(R)₂, —CN, and optionally substituted C₁₋₆ aliphatic; and
m is 0, 1, 2, or 3.

37. The compound of any one of claims 2-36, wherein L² is a covalent bond.

38. The compound of any one of claims 2-37, wherein LBM or 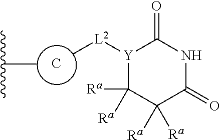
moiety is selected from:

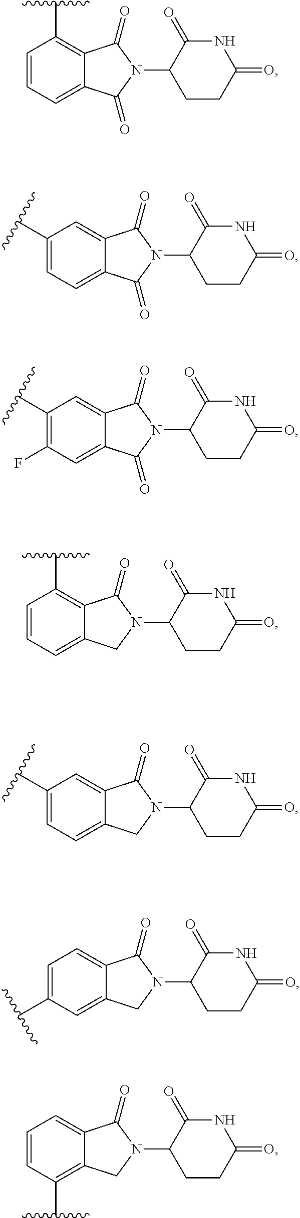

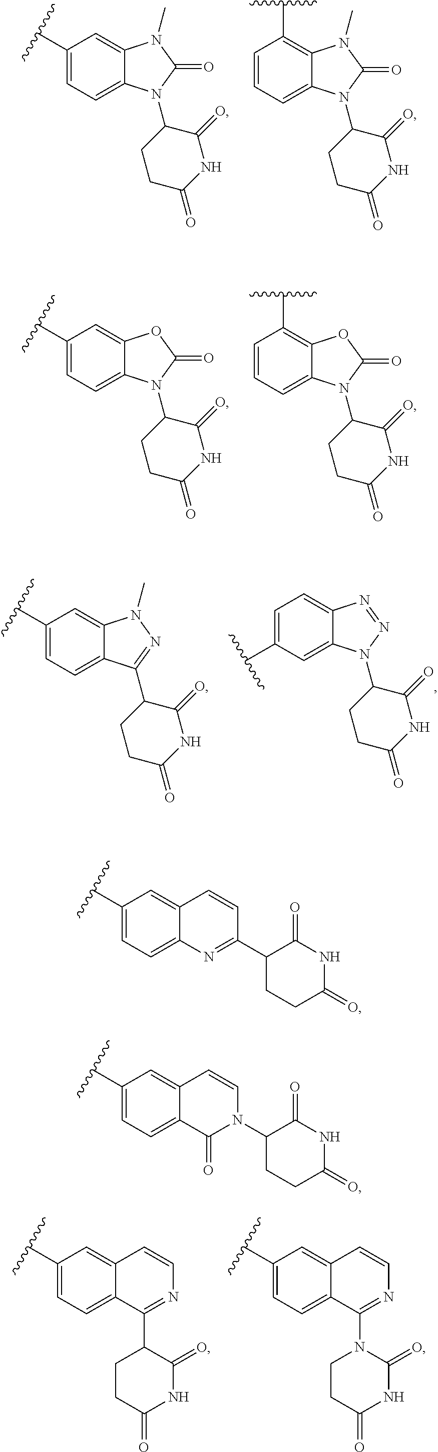

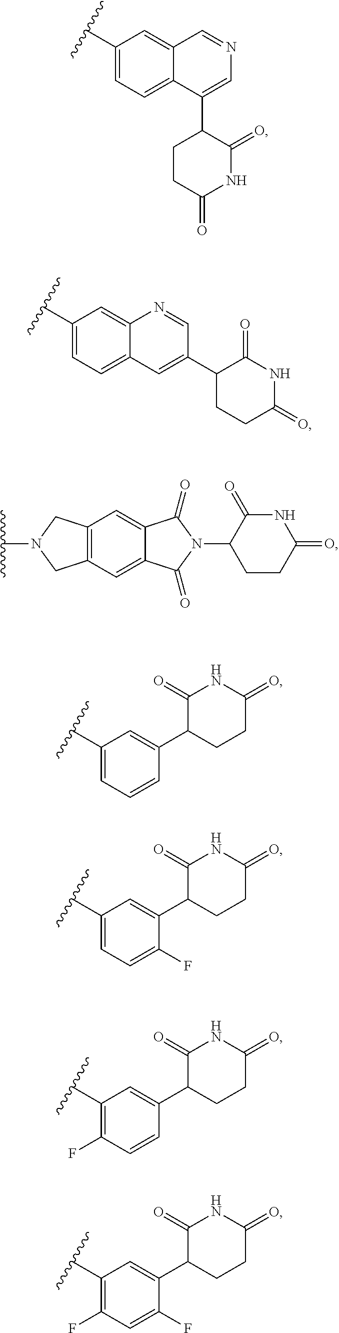

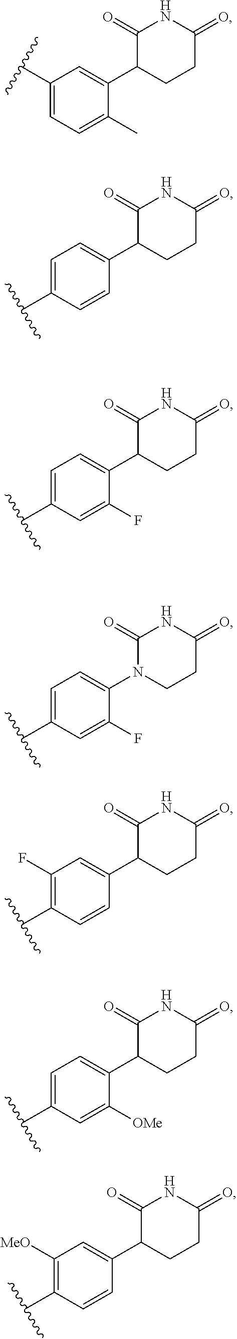

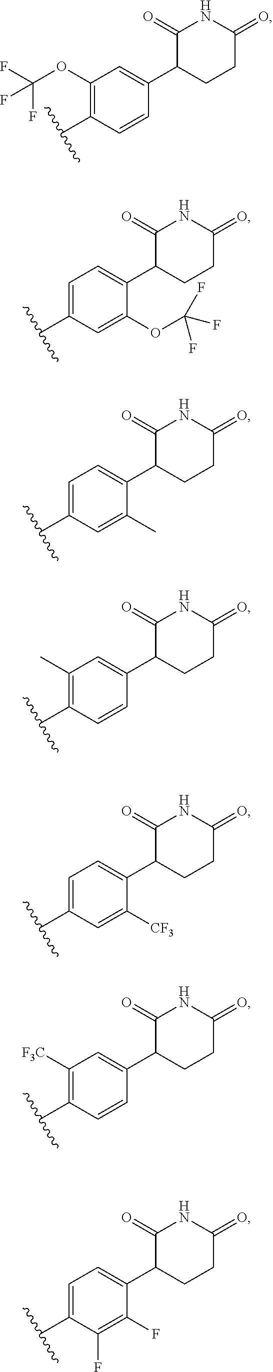

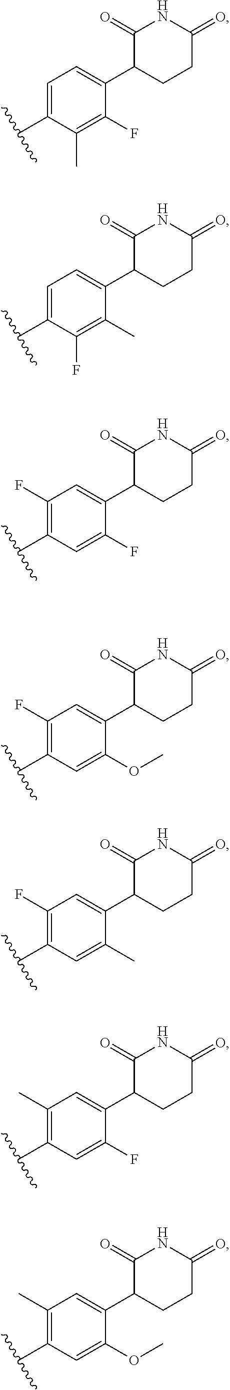

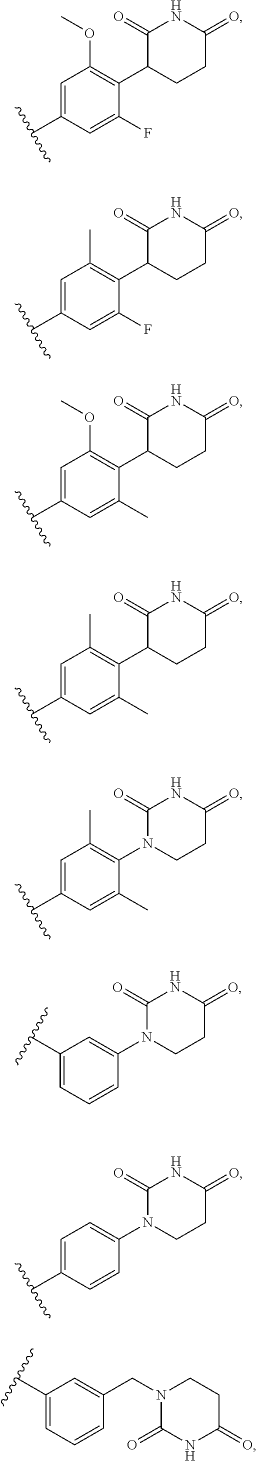

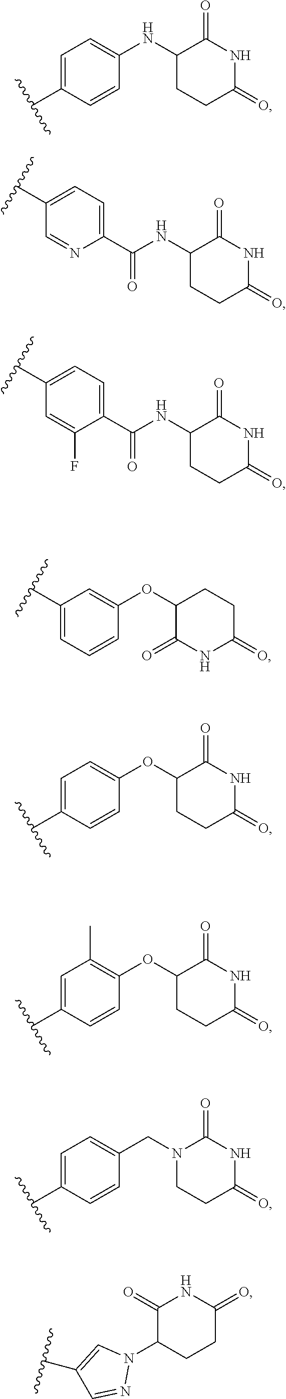

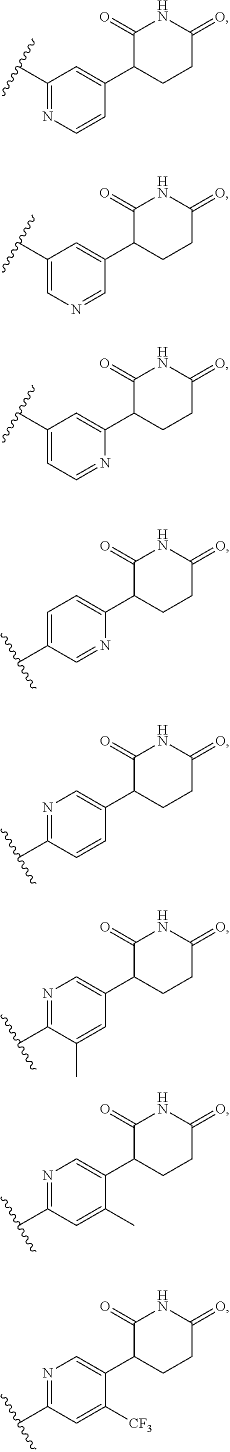

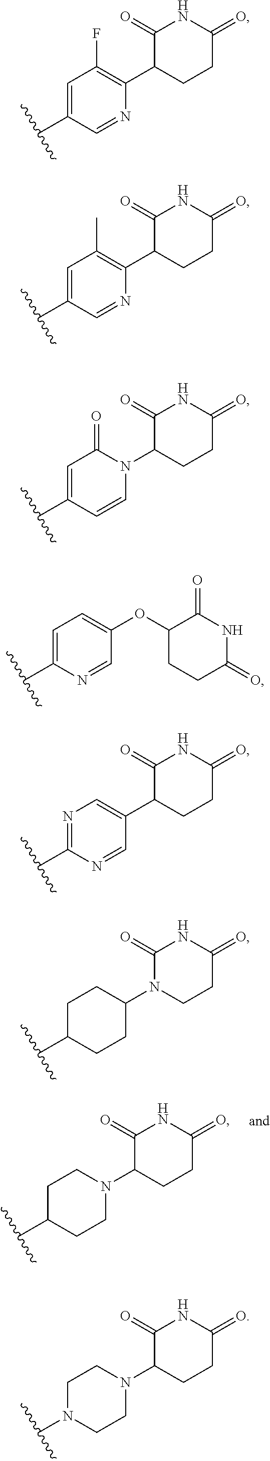

39. The compound of any one of claims 2-35, wherein LBM is

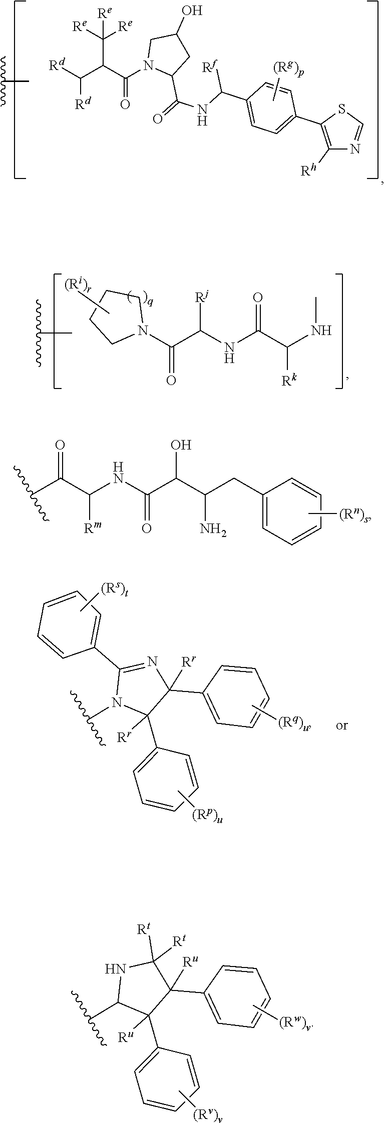

40. The compound of any one of claims 2-3, wherein PBM is

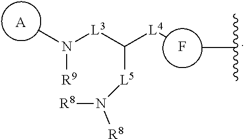

41. The compound of any one of claims 2-3 or 40, wherein Ring F is an optionally substituted 3- to 10-membered ring having 0-4 heteroatoms independently selected from N, O, and S.

42. The compound of claim 41, wherein Ring A is selected from

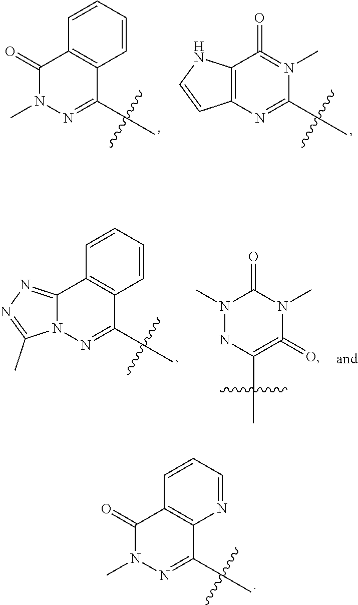

43. The compound of any one of claims 40-42, wherein LBM is

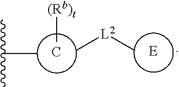

44. The compound of any one of the preceding claims, wherein the compound is in a pharmaceutically acceptable salt form.

45. The compound of any one of the preceding claims, wherein the stereochemical purity of each chiral center in PBM is at least about 90%.

46. The compound of any one of the preceding claims, wherein the stereochemical purity of each chiral center is at least about 90%.

47. The compound of any one of the preceding claims, wherein purity of the compound by qNMR is at least about 90% (wt %).

48. A pharmaceutical composition comprising a compound of any one of any one of the preceding claims, or a pharmaceutically acceptable salt thereof, and a pharmaceutically acceptable carrier.

49. The composition of claim 48, wherein the composition further comprises one or more stereoisomers of the compound, wherein the percentage of the compound, among the total of the compound and all its stereoisomers in the composition, is about or at least about 90%, or wherein the percentage of the compound, among the total of the compound and all its stereoisomers in the composition, is about or at least about 20%-80%.

50. The composition of any one of claims 48-49, wherein the composition comprises a buffer or components thereof, preferably wherein the buffer is a citrate buffer and/or the pH of the buffer is about 2-6.5.

51. The composition of any one of claims 48-50, wherein the composition comprises a cyclodextrin.

52. The composition of any one of claims 48-50, wherein the composition is substantially free of cyclodextrin.

53. A method of preparing a pharmaceutical composition of claim 48, comprising:
providing the compound of any one of claims 1-47, or a pharmaceutically acceptable salt thereof; and
formulating the compound with suitable excipients to give the pharmaceutical composition.

54. A method for reducing KAT2A and/or KAT2B in a system, comprising administering or delivering to the system an effective amount of a compound or composition of any one of the preceding claims; or a pharmaceutically acceptable salt thereof, or a composition of any one of the preceding claims.

55. A method for modulating a dysregulated cell state associated with KAT2A and/or KAT2B, comprising administering or delivering to the cell an effective amount of a compound of any one of the preceding claims, or a pharmaceutically acceptable salt thereof, or a composition of any one of the preceding claims.

56. The method of claim 55, wherein the dysregulated cell state is of cancer, e.g., of acute myeloid leukemia, small cell lung cancer, or neuroendocrine prostate cancer.

57. The method of any one of claims 55-56, wherein the method induces or promotes conversion of the dysregulated cell state into another cell state; wherein the dysregulated cell state is more proliferative than the another cell state; wherein the dysregulated cell state is less differentiated than the another cell state; wherein the dysregulated cell state is or comprises blocked or reduced differentiation; wherein the dysregulated cell state is or comprises de-differentiation: wherein the dysregulated cell state is or comprises trans-differentiation; wherein the dysregulated cell state is or comprises a dedifferentiated neuroendocrine cell state; and/or wherein the another cell state is or comprises a more differentiated epithelial cell state.

58. The method of any one of claims 55-57, wherein the another cell state comprises increased levels of CD11b, CD14, CD15, and/or CD86 expression; wherein the method increases myeloid differentiation, and/or decreases stem, translation and replication; and/or wherein the method increases myeloid differentiation, and/or decreases stem, translation and/or replication GSEA signatures.

59. The method of any one of claims 55-58, wherein the method increases epithelial GSEA signatures; and/or wherein the method increases neural GSEA signatures; and/or wherein the method increases monocytic differentiation; and/or wherein the method increases neural differentiation; and/or wherein the method increases epithelial differentiation; and/or wherein the method increases level of CD11b+ cells; and/or wherein the method increases level of CD14+ cells; and/or wherein the method increases level of CD15+ cells; and/or wherein the method increases level of CD86+ cells.

60. A method for reducing histone acetylation in a system, comprising administering or delivering to the system an effective amount of a compound of any one of the preceding claims, or a pharmaceutically acceptable salt thereof, or a composition of any one of the preceding claims.

61. The method of claim 60, wherein H3K9 acetylation is reduced.

62. A method of degrading KAT2 in a subject, comprising administering the compound of any one of the preceding claims, or a pharmaceutically acceptable salt thereof, or the pharmaceutical composition of any one of the preceding claims, to a subject in need thereof.

63. A method for inducing or promoting cell differentiation in a system, comprising administering or delivering to the system an effective amount of a compound of any one of the preceding claims, or a pharmaceutically acceptable salt thereof, or a composition of any one of the preceding claims; or a method for reducing or blocking de-differentiation in a system, comprising administering or delivering to the system an effective amount of a compound of any one of the preceding claims, or a pharmaceutically acceptable salt thereof, or a composition of any one of the preceding claims; or a method for reducing or blocking trans-differentiation in a system, comprising administering or delivering to the system an effective amount of a compound of any one of the preceding claims, or a pharmaceutically acceptable salt thereof, or a composition of any one of the preceding claims.

64. The method of any one of claims 60-63, wherein the system comprises or expresses KAT2A and/or KAT2B.

65. The method of any one of claims 54-64, wherein a system is or comprises a cell.

66. The method of any one of claims 54-64, wherein a system is or comprises a cancer cell.

67. The method of any one of claims 54-64, wherein a system is or comprises an acute myeloid leukemia cell.

68. A method for treating a disease, disorder, or condition, comprising administering or delivering to a subject suffering therefrom an effective amount of a compound or composition of any one of the preceding claims.

69. The method of claim 68, wherein the disease, disorder, or condition is associated with KAT2A and/or KAT2B.

70. The method of any one of claims 68-69, wherein the disease, disorder, or condition is cancer, or wherein the disease, disorder, or condition is selected from acute myeloid leukemia, neuroblastoma, non-small cell lung cancer, small cell lung cancer, colorectal cancer, melanoma, prostate cancer, neuroendocrine prostate cancer, castration resistant prostate cancer, high-grade neuroendocrine carcinomas, high-grade neuroendocrine carcinomas in GI tract, high-grade neuroendocrine carcinomas in pancreas, and pancreatic cancer.

71. A compound, composition, or method of any one of Embodiments 1-689.
